(19) United States
(12) Patent Application Publication
(10) Pub. No.: US 2025/0230138 A1
(43) Pub. Date: Jul. 17, 2025

(54) COMPOUNDS HAVING A T-STRUCTURE FORMED BY AT LEAST FOUR CYCLES FOR USE IN THE TREATMENT OF CANCER AND OTHER INDICATIONS

(71) Applicants: TheRas, Inc., South San Francisco, CA (US); Leidos Biomedical Research, Inc., Frederick, MD (US); Lawrence Livermore National Security, LLC, Livermore, CA (US)

(72) Inventors: Rui Xu, Dallas, TX (US); Bin Wang, Dallas, TX (US); Eli Wallace, Loveland, CO (US); Paul Wehn, New Haven, CT (US); Pedro J. Beltran, Orinda, CA (US); James Stice, Clayton, CA (US); Kerstin Sinkevicius, Lincoln, MA (US); Yue Yang, Foster City, CA (US); Paola Bisignano, San Francisco, CA (US); Felice Lightstone, Fremont, CA (US); Dhirendra Kumar Simanshu, Frederick, MD (US); David Michael Turner, Frederick, MD (US); Daniel J. Czyzyk, Frederick, MD (US); Swapnil Singh, Frederick, MD (US); Jayasudhan Reddy Yerabolu, Frederick, MD (US); Francis Patrick McCormick, Palo Alto, CA (US)

(21) Appl. No.: 18/836,555

(22) PCT Filed: Feb. 7, 2023
(86) PCT No.: PCT/US23/12521

§ 371 Date: Aug. 7, 2024

Related U.S. Application Data
(60) Provisional application No. 63/416,772, filed on Oct. 17, 2022.
(60) Provisional application No. 63/307,882, filed on Feb. 8, 2022.

(51) Int. Cl. C07D 401/04; A61K 31/4365; A61K 31/437; A61K 31/4375; A61K 31/438; A61K 31/444; A61K 31/4545; A61K 31/4725; A61K 31/496; A61K 31/497; A61K 31/4985; A61K 31/501; A61K 31/502; A61K 31/5025; A61K 31/506; A61K 31/519; A61K 31/541; A61K 31/55; A61K 31/551; A61P 35/00; C07D 401/14; C07D 487/04; C07D 495/04; C07D 519/00

(52) U.S. Cl. CPC C07D 401/04; A61K 31/4365; A61K 31/437; A61K 31/4375; A61K 31/438; A61K 31/444; A61K 31/4545; A61K 31/4725; A61K 31/496; A61K 31/497; A61K 31/4985; A61K 31/501; A61K 31/502; A61K 31/5025; A61K 31/506; A61K 31/519; A61K 31/541; A61K 31/55; A61K 31/551; A61P 35/00; C07D 401/14; C07D 487/04; C07D 495/04; C07D 519/00

(57) ABSTRACT

Provided herein are compounds and compositions thereof that may be capable of disrupting, interrupting, and/or preventing an interaction between a small GTPase protein and a PI3K protein (e.g., PI3Ka). The present disclosure also provides methods of treating cancers and other indications with such compounds or compositions thereof.

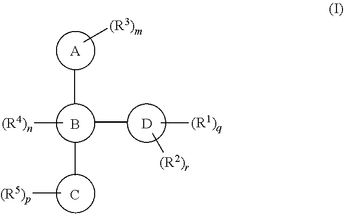

### RELATED APPLICATIONS

This application claims priority to and benefit of U.S. Application No. 63/307,882, filed Feb. 8, 2022, and U.S. Application No. 63/416,772, filed Oct. 17, 2022, the entire contents of each of which are hereby incorporated by reference.

### GOVERNMENT SUPPORT

This invention was made with government support under Contract No. DE-AC52-07NA27344 awarded by the United States Department of Energy. The government has certain rights in the invention.

### BACKGROUND

An estimated over 600,000 Americans will have died from cancer in 2021, corresponding to more than 1600 deaths per day (Cancer Facts and Figures 2021). The greatest number of deaths are from cancers of the lung, prostate, and colorectum in men, and cancers of the lung, breast, and colorectum in women. Almost one-quarter of all cancer deaths are due to lung cancer, 82% of which is directly caused by cigarette smoking. The 5-year survival rate for lung cancer patients is only about 20%.

The aberrant activation of the phosphoinositide 3-kinase (PI3K) is one of the most frequent oncogenic events across human cancers, and its inhibition is an attractive therapeutic approach in treating cancers. PI3Ks signal downstream of receptor tyrosine kinases (RTKs), G protein-coupled receptors (GPCRs), and RAS proteins to regulate a large number of cellular activities, including metabolism, proliferation, and migration.

The frequency of PI3K oncogenic events has fueled the development and testing of PI3K inhibitors. Most PI3K inhibitors that have entered clinical development thus far are reversible, ATP-competitive kinase inhibitors. Despite considerable efforts, the clinical outcome of PI3K inhibitor-based treatments for solid tumors has been disappointing, mainly due to intolerable toxicity and drug resistance.

### SUMMARY

The present disclosure provides new therapeutic modalities for treating cancers and other indications (e.g., cancers and other indications associated with and/or characterized by aberrant activation of PI3K). The present disclosure encompasses the recognition that a therapeutic agent (e.g., a therapeutic agent comprising a small molecule, e.g., a compound provided herein) that disrupts, inhibits, and/or prevents an interaction between a PI3K protein (e.g., PI3Kα) and a small GTPase (e.g., Rac1, CDC42, or a RAS protein such as KRAS, NRAS, HRAS, RRAS, RRAS2, MRAS, or RIT1) may be particularly useful for treating, ameliorating, delaying the progress of, ameliorating or eliminating a symptom of, and/or inhibiting a cancer and/or other indication (e.g., an indication associated with and/or characterized by aberrant activation of PI3K). Without wishing to be bound by theory, therapeutic agents provided herein (e.g., therapeutic agents comprising small molecules, e.g., compounds provided herein) may be capable of binding to a PI3K protein (e.g., PI3Kα) while displaying (i) no or minimal binding to a small GTPase (e.g., Rac1, CDC42, or a RAS protein such as KRAS, NRAS, HRAS, RRAS, RRAS2, MRAS, or RIT1) and/or (ii) no substantial effect on the kinase activity of the PI3K protein (e.g., PI3Kα). In some embodiments, such therapeutic agents may provide advantages such as improved efficacy or reduced side effects as compared to, e.g., ATP-competitive PI3K kinase inhibitors, as described herein. For example, in some embodiments, such therapeutic agents may provide reduced instances of hyperglycemia and/or hyperinsulinemia relative to PI3K kinase inhibitors.

The present disclosure provides compounds (including in any available forms, such as salt forms) useful in disrupting, inhibiting, and/or preventing an interaction between a PI3K protein (e.g., PI3Kα) and a small GTPase (e.g., Rac1, CDC42, or a RAS protein such as KRAS, NRAS, HRAS, RRAS, RRAS2, MRAS, or RIT1). In some embodiments, the present disclosure provides compounds capable of binding a PI3Kα protein, such that (i) the interaction between a small GTPase (e.g., Rac1, CDC42, or a RAS protein such as KRAS, NRAS, HRAS, RRAS, RRAS2, MRAS, or RIT1) and the PI3Kα protein is disrupted, inhibited, and/or prevented; and/or (ii) the kinase activity of the PI3Kα protein is not inhibited. In some embodiments, such compounds are useful for treating a cancer or other indication, as described herein.

In an aspect, the present disclosure provides a compound of Formula I:

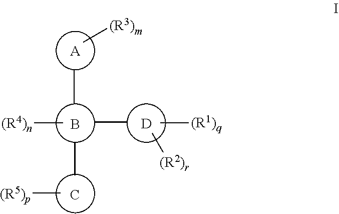

or a salt (e.g., a pharmaceutically acceptable salt) thereof, wherein each of Ring A, Ring B, Ring C, Ring D, R¹, R², R³, R⁴, R⁵, m, n, p, q, and r is as defined herein. In some embodiments, a compound of Formula I is capable of binding a PI3Kα protein, such that (i) the interaction between a small GTPase (e.g., Rac1, CDC42, or a RAS protein such as KRAS. NRAS, HRAS, RRAS, RRAS2, MRAS, or RIT1) and the PI3Kα protein is disrupted, inhibited, and/or prevented; and/or (ii) the kinase activity of the PI3Kα protein is not inhibited.

In another aspect, the present disclosure provides a pharmaceutical composition comprising a compound provided herein (e.g., a compound of Formula I), or a salt (e.g., a pharmaceutically acceptable salt) thereof, together with a pharmaceutically acceptable carrier.

In another aspect, the present disclosure provides a method of inhibiting, disrupting, and/or preventing an interaction between a PI3Kα protein and a small GTPase (e.g., Rac1, CDC42, or a RAS protein such as KRAS, NRAS, HRAS, RRAS, RRAS2, MRAS, or RIT1) such that the kinase activity of the PI3Kα protein is not inhibited. In some embodiments, the PI3Kα protein is in a cell, such as in a cell of a human or animal subject (e.g., as described herein).

In a further aspect, the present disclosure provides a method of treating, ameliorating, delaying the progress of, ameliorating or eliminating a symptom of, and/or inhibiting a cancer and/or other indication (e.g., an indication associated with and/or characterized by aberrant activation of PI3K) comprising administering a compound provided herein (e.g., a compound of Formula I), or a salt (e.g., a pharmaceutically acceptable salt) thereof.

In a related aspect, the present disclosure provides a use of a compound provided herein (e.g., a compound of Formula I), or a salt (e.g., a pharmaceutically acceptable salt) thereof, in the manufacture of a medicament for the treatment, amelioration, or inhibition of a cancer or other indication (e.g., an indication associated with and/or characterized by aberrant activation of PI3K). The present disclosure also provides a compound (e.g., a compound of Formula I), or a salt (e.g., a pharmaceutically acceptable salt) thereof, for use as a medicament, which medicament may be used in the treatment, amelioration, or inhibition of a cancer or other indication (e.g., an indication associated with and/or characterized by aberrant activation of PI3K).

### BRIEF DESCRIPTION OF THE DRAWINGS

FIG. 1 shows the effect of inhibiting PI3Kα with a PI3Kα kinase inhibitor such as alpelisib in normal cells (left panel) and tumor cells (right panel). In normal cells, inhibiting PI3Kα blocks normal cell signaling resulting in dose-limiting hyperglycemia and insulin-driven resistance. The right panel shows an alternative scheme of using a PI3Kα breaker to inhibit the PI3Kα:RAS protein:protein interaction in tumor cells. This mechanism should avoid hyperglycemia and insulin-driven resistance by selectively targeting tumor cells and may provide multiple therapeutic advantages. The observation that mutations in the RAS binding domain that impair the PI3Kα:RAS interaction blocks oncogene-driven non-small cell lung cancer (NSCLC) tumor growth in vivo and have no effect on glucose metabolism support the potential of this mechanism.

FIGS. 2A and 2B show that a PI3Kα breaker can have broad in vitro activity. FIG. 2A shows one third of human tumor cell lines tested have a pAKT IC50<100 nanomolar (nM) and depend on the RAS:PI3Kα interaction for activation of Akt signaling. In particular, 29 of 50 (58%) G12X cell lines have a pAKT IC50<100 nM. Bioinformatic analyses in FIG. 2B show that cell lines with PIK3CA helical mutations are particularly sensitive to the PI3Kα breaker without inducing hyperglycemia; in addition, 78% of HER2 amplified lines are sensitive to the PI3Kα breaker.

FIGS. 3A-3D shows that PI3Kα breakers are orally bioavailable and can achieve near complete inhibition of signaling in tumors without risk of hyperglycemia or hyperinsulinemia. FIG. 3A shows the pharmacokinetics of a PI3Kα breaker compound in mouse. FIG. 3B shows dose response pharmacodynamics for a PI3Kα breaker (“BBO”) and the PI3Kα kinase domain inhibitor alpelisib in a BT474 cell line-derived xenograft model. The dose response pharmacodynamics demonstrate that near complete target inhibition is achieved at 100 mg/kg of PI3Kα breaker. (One-way ANOVA with Dunnett's test vs. vehicle; *=p<0.01; **=p<0.0001). From left to right along x-axis in FIG. 3B: vehicle, 30 mg/kg BBO, 100 mg/kg BBO, 300 mg/kg BBO, 50 mg/kg alpelisib. FIGS. 3C and 3D demonstrate that unlike PI3Kα kinase domain inhibitors, PI3Kα breaker compounds do not affect glucose metabolism. FIG. 3C shows blood glucose levels at the time of and after administration of a single dose of vehicle, the PI3Kα kinase domain inhibitor alpelisib, and a PI3Kα breaker (Compound 58, “BBO”) in fasted male mice after an oral glucose tolerance test. Hyperglycemia is observed in alpelisib but not in PI3Kα breaker treated mice. (One-way ANOVA with Dunnett's test vs vehicle, *p<0.05, **p<0.01, ***p<0.001, ****p<0.0001) FIG. 3D shows insulin levels at the time of and 240 minutes after administration of a single oral dose of vehicle, 50 mg/kg alpelisib, or 300 mg/kg Compound 58 (“BBO”) in fed male mice. Hyperinsulinemia is observed in alpelisib but not PI3Kα breaker treated mice. (One-way ANOVA with Tukey's multiple comparisons test vs all other groups: *p<0.0001) (IV=intravenous administration; PO=per os or oral administration; ng/mL=nanograms per milliliters; mg/kg=milligrams per kilogram; mg/dL=milligrams per deciliter; min=minutes; CDX=cell line derived xenograft; QD=quaque die or once daily, PK=pharmacokinetics)

FIGS. 4A-4D show that strong efficacy is observed in xenograft models with KRAS G12X mutations, with or without PIK3CA mutations. FIG. 4A shows changes in tumor volume in a KYSE-410 cell line-derived xenograft model featuring a KRAS G12C mutation and HER2 amplification. Dose dependent, significant efficacy was observed and there was tumor regression with 100 mg/kg of PI3Kα breaker (“BBO”). FIG. 4B shows changes in tumor volume in a GP2d cell line-derived xenograft model featuring a KRAS G12D mutation and a PIK3CA H1047L mutation. Significant tumor volume reduction is observed with 10 mg/kg and higher amounts of PI3Kα breaker (“BBO”). FIG. 4C shows changes in tumor volume in a SNU-601 cell line-derived xenograft model featuring a KRAS G12D mutation and a PIK3CA E542K mutation. Significant tumor volume reduction is observed with 10 mg/kg and higher amounts of PI3Kα breaker (“BBO”). FIG. 4D shows changes in tumor volume in a SNU-16 cell line-derived xenograft model that features a KRAS G12D mutation. Significant tumor volume reduction is observed with 10 mg/kg and higher amounts of PI3Kα breaker (“BBO”). (p<0.0005 two-way repeated measures ANOVA vs. vehicle; all groups dosed per os (PO, orally), once daily (QD) (mm³=cubic millimeters; mg/kg=milligrams per kilogram)

### DETAILED DESCRIPTION

### Compounds and Definitions

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric, diastereomeric, atropisomeric, or epimeric) forms of the structure, as well as all geometric or conformational isomeric forms of the structure. For example, the R and S configurations of each stereocenter are contemplated as part of the disclosure; and the D- and L-isomers of each compound are contemplated as part of the disclosure. Therefore, single stereochemical isomers, as well as enantiomeric, diastereomeric, atropisomeric, and geometric (or conformational) mixtures of provided compounds are within the scope of the disclosure. For example, in some cases, Table 1 and Table 2 shows one or more stereoisomers of a compound, and unless otherwise indicated, represents each stereoisomer alone and/or as a mixture. The present disclosure includes all cis, trans, syn, anti, entgegen (E), and zusammen (Z) isomers, as well as mixtures thereof. Individual stereoisomers of compounds can be prepared synthetically from commercially available starting materials that contain chiral centers or by preparation of mixtures of enantiomeric products followed by separation, such as conversion to a mixture of diastereomers followed by separation via, e.g., recrystallization, chromatographic techniques, direct separation of enantiomers on chiral chromatographic columns, or any other appropriate method. Starting compounds of particular stereochemistry are either commercially available or can be made and resolved by various techniques. Unless otherwise stated, all tautomeric forms (e.g., rapidly interconverting forms) of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

Aliphatic: The term “aliphatic” or “aliphatic group”, as used herein, means a straight-chain (i.e., unbranched) or branched, substituted or unsubstituted hydrocarbon chain that is completely saturated or that contains one or more units of unsaturation (e.g., multiple bonds, such as double or triplebonds). Unless otherwise specified, aliphatic groups contain 1-12 aliphatic carbon atoms. In some embodiments, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms.

Alkyl: The term “alkyl”, used alone or as part of a larger moiety, refers to a saturated, optionally substituted straight or branched hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Examples of alkyl groups include methyl, ethyl, propyl (e.g., n-propyl), isopropyl, n-butyl, t-butyl, isobutyl, sec-butyl, pentyl, isoamyl, hexyl, heptyl, octyl, and nonyl. The term “alkylene,” as used herein, alone or in combination, refers to a bivalent, saturated, optionally substituted straight or branched hydrocarbon, such as methylene (—CH₂—).

Alkenyl: The term “alkenyl”, used alone or as part of a larger moiety, refers to an optionally substituted straight or branched hydrocarbon chain having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Examples of alkenyl groups include ethenyl, propenyl, 2-methylpropenyl, 1,4-butadienyl, butenyl, pentenyl, hexenyl, and heptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Examples of alkynyl groups include ethynyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, and 1,3,5-hexatriynyl.

Aryl: The term “aryl” used alone or as part of a larger moiety as in “aralkyl,” “aralkoxy,” or “aryloxyalkyl,” refers to monocyclic or bicyclic ring systems having a total of six to fourteen ring members, wherein at least one ring in the system is aromatic and wherein each ring in the system contains 3 to 7 ring members. A bicyclic ring system may comprise first and second rings that are fused together and/or share one or more atoms. The term “aryl” may be used interchangeably with the term “aryl ring(s).” In certain embodiments of the present invention, “aryl” refers to an aromatic ring system. Examples of aryl groups include phenyl, biphenyl, naphthyl, anthracyl and the like, which may bear one or more substituents as defined herein. Also included within the scope of the term “aryl,” as it is used herein, is a group in which an aromatic ring is fused to one or more non-aromatic rings, such as indanyl or tetrahydronaphthyl, and the like. Unless otherwise specified, “aryl” groups are hydrocarbons.

Carbocyclyl: The terms “carbocyclyl,” “carbocycle,” and “carbocyclic ring” as used herein, refer to saturated or partially unsaturated cyclic aliphatic monocyclic, bicyclic, or polycyclic ring systems, as described herein, having from 3 to 14 members, wherein the aliphatic ring system is optionally substituted as described herein. A carbocycle may comprise fused ring systems, bridged ring systems, and/or spiro ring systems (e.g., a system including two rings sharing a single carbon atom). Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₆-C₁₀ bicyclic hydrocarbon that is completely saturated or that contains one or more units of unsaturation, but which is not aromatic. The term “cycloalkyl” refers to an optionally substituted saturated ring system of about 3 to about 10 ring carbon atoms. In some embodiments, cycloalkyl groups have 3-6 carbons. Examples of monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Examples of monocyclic cycloalkenyl rings include cyclopentenyl, cyclohexenyl, and cycloheptenyl.

Halogen: The term “halogen” or “halo” means F, Cl, Br, or I.

Heteroaryl: The terms “heteroaryl”, “heteroaromatic”, and “heteroar-,” used alone or as part of a larger moiety, e.g., “heteroaralkyl,” or “heteroaralkoxy,” refer to groups having 5 to 14 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings. Examples of bicyclic heteroaromatic groups include indolyl, isoindolyl, benzothienyl, benzofuranyl, indazolyl, indolizinyl, benzimidazolyl, benzthiazolyl, benzotriazolyl, benzoxazolyl, benzoxadiazolyl, benzothiadiazolyl, tetrazolopyridazinyl, thienopyridinyl, furopyridinyl, pyrrolopyridinyl, chromonyl, coumarinyl, quinolyl, isoquinolyl, cinnolinyl, phthalazinyl, quinazolinyl, quinoxalinyl, 4H-quinolizinyl, tetrahydroquinolinyl, and tetrahydroisoquinolinyl. Examples of tricyclic heterocyclic groups include carbazolyl, phenanthrolinyl, dibenzofuranyl, acridinyl, phenazinyl, phenanthridinyl, phenothiazinyl, phenoxazinyl, and xanthenyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. It will be appreciated that certain tautomeric forms of a heteroaryl ring can exist and are encompassed by the term “heteroaryl.” Such tautomeric forms include, for example, pyridin-2(1H)-one.

Heteroatom: The term “heteroatom” means one or more of oxygen, sulfur, nitrogen, phosphorus, or silicon (including, any oxidized form of nitrogen, sulfur, phosphorus, or silicon); the quaternized form of any basic nitrogen or a substitutable nitrogen of a heterocyclic ring, for example N (as in 3,4-dihydro-2H-pyrrolyl), NH (as in pyrrolidinyl) or NR⁺ (as in N-substituted pyrrolidinyl)). In some embodiments, a heteroatom is selected from oxygen, sulfur, and nitrogen.

Heterocycle: As used herein, the terms “heterocycle,” “heterocyclyl,” “heterocyclic radical,” and “heterocyclic ring” are used interchangeably and refer to a stable 3- to 8-membered monocyclic or 5- to 10-membered bicyclic heterocyclic moiety that is either saturated or partially unsaturated, and having, in addition to one or more carbon atoms, one or more, preferably one to four, heteroatoms, as defined above. When used in reference to a ring atom of a heterocycle, the term “nitrogen” includes a substituted nitrogen. A heterocyclic ring can be attached to its pendant group at any heteroatom or carbon atom that results in a stable structure and any of the ring atoms can be unsubstituted or substituted with one or more substituents (e.g., as described herein). Examples of such saturated or partially unsaturated heterocyclic radicals include tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted with a heterocyclyl, wherein the alkyl and heterocyclyl portions independently are unsubstituted or substituted with one or more substituents (e.g., as described herein).

Partially Unsaturated: As used herein, the term “partially unsaturated”, when referring to a ring moiety, means a ring moiety that includes at least one double or triple bond between ring atoms. The term “partially unsaturated” is intended to encompass rings having multiple sites of unsaturation, but is not intended to include aryl or heteroaryl moieties, as herein defined.

Patient or Subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, hamsters, guinea pigs, cats, dogs, goats, pigs, sheep, cows, deer, horses, non-human primates, and/or humans). In some embodiments, a patient or subject is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Prevent or prevention: As used herein, when used in connection with the occurrence of a disease, disorder, and/or condition, “prevent” or “prevention” refers to reducing the risk of developing the disease, disorder, or condition; delaying onset of one or more characteristics or symptoms of the disease, disorder, or condition; and/or preventing escalation of a disease, disorder, or condition. Prevention of a disease, disorder, or condition may involve complete protection from disease and/or prevention of disease progression (e.g., to a later stage of the disease, disorder, or condition). For example, prevention of a disease may not mean complete foreclosure of any effect related to the diseases at any level, but instead may mean prevention of the symptoms of a disease, disorder, or condition to a clinically significant or detectable level. Prevention may be considered complete when onset of a disease, disorder or condition has been delayed for a predefined period of time.

Substituted or Optionally Substituted: As described herein, compounds of this disclosure may contain “optionally substituted” moieties (e.g., moieties bearing one or more substituents). In general, the term “substituted,” whether preceded by the term “optionally” or not, means that one or more hydrogens of the designated moiety are replaced with a suitable substituent. “Substituted” applies to one or more hydrogens that are either explicit or implicit from the structure

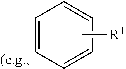

refers to at least

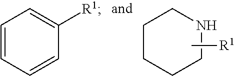

and refers to at least

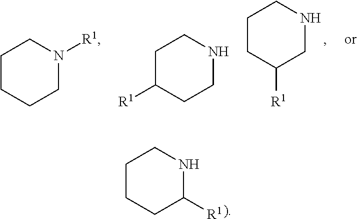

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R⁰)C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂, —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include =O and =S.

Suitable divalent substituents on a saturated carbon atom of an “optionally substituted” group include the following: =O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Treat: As used herein, the term “treat” (also “treatment” or “treating”) refers to any administration of a therapy (e.g., therapeutic agent) that partially or completely alleviates, ameliorates, relives, inhibits, delays onset of, reduces severity of, and/or reduces incidence of one or more symptoms, features, and/or causes of a particular disease, disorder, and/or condition. Treatment may also refer to any other indicia of success in the treatment or amelioration of an injury, pathology, disease, disorder, or condition, including any objective or subjective parameter such as abatement; remission; diminishing of symptoms or making the injury, pathology, disease, disorder, or condition more tolerable to the patient; slowing in the rate of degeneration or decline; making the final point of degeneration less debilitating; and/or improving a patient's physical or mental well-being. The treatment or amelioration of symptoms can be based on objective or subjective parameters, including the results of a physical examination, neuropsychiatric exams, and/or a psychiatric evaluation. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition.

Composition: As used herein, the term “composition” refers to a discrete physical entity that comprises one or more specified components (e.g., a product comprising one or more specified ingredients (e.g., in specified amounts) or a product that results, directly or indirectly, from combination of specified ingredients in specified amounts). Unless otherwise specified, a composition may be of any form—e.g., gas, gel, liquid, solid, etc. A composition may comprise one or more pharmaceutically acceptable components, such as a carrier, diluent, or excipient. By “pharmaceutically acceptable” it is generally meant the carrier, diluent, or excipient must be compatible with the other ingredients of the formulation and not deleterious to the recipient thereof. For example, a “pharmaceutically acceptable excipient” refers to a substance that aids the administration of an active agent to and absorption by a subject.

### PI3K and Small GTPase Proteins

The aberrant activation of the phosphoinositide 3-kinase (PI3K) is one of the most frequent oncogenic events across human cancers, and its inhibition is an attractive therapeutic approach in treating cancers. PI3Ks signal downstream of receptor tyrosine kinases (RTKs), G protein-coupled receptors (GPCRs), and RAS proteins to regulate a large number of cellular activities, including metabolism, proliferation, and migration. Upon activation, PI3K catalyzes the synthesis of the second messenger phosphatidylinositol (3,4,5)-trisphosphate (PIP3) by phosphorylating phosphatidylinositol 4,5-bisphosphate (PIP2). Signaling proteins such as Ser/Thr kinase AKT (e.g., Protein Kinase B (PKB)) can bind to PIP3 and thereby localize to the cell membrane. Phosphorylated AKT activates or inhibits several signaling proteins through direct phosphorylation including the mammalian target of rapamycin complex 1 (mTORC1), which acts as a regulator of cell growth and survival pathways, cyclin D1, GSK3(B), BAD, MDM2, FOXO, TSC1/2, and PRAS40. Phosphatase and tensin homologue deleted on chromosome 10 (PTEN) regulates this pathway by dephosphorylating PIP3 to PIP2 and thus prevents activation of downstream kinases.

Based on the sequence homology and substrate preference, PI3Ks have been grouped into three separate classes (e.g., classes I, II, and III). Class I PI3Ks are further divided into two subclasses, IA and IB depending on their modes of regulation. Class IA PI3Ks are heterodimers comprising p110 catalytic and p85 regulatory subunits, and are most clearly implicated in human cancer. Class IA PI3K contains p110α, p110β, and p110δ catalytic subunits produced from different genes (PIK3CA. PIK3CB, and PIK3CD, respectively), while p110γ produced by PIK3CG represents the only catalytic subunit in class IB PI3K. The expression of PI3K isoforms (e.g., PI3Kα, PI3Kβ, PI3Kδ, and PI3Kγ) is specific to cell types. The p110α and β isoforms are expressed in all cell types, whereas p110δ expression is mainly confined to leukocytes. The p110γ isoform is expressed primarily in the myeloid cell lineage.

PIK3CA gene encodes the 1068 amino acid p110α protein that contains five domains: an N-terminal adaptor binding domain (ABD) that binds to regulatory subunit p85α, a RAS-binding domain (RBD), a C2 domain, a helical domain, and a kinase catalytic domain. RAS contributes directly to the activation of the PI3K pathway through direct binding of RAS proteins (e.g., HRAS, NRAS, and KRAS) to a RAS-binding domain (RBD) in the p110α catalytic subunit of PI3Kα. Activating mutations in the KRAS and PIK3CA genes are frequently detected in cancer, making these two proteins important targets for drug discovery. Somatic missense mutations in the PIK3CA gene have been reported in many human cancer types including breast, colon, liver, stomach, endometrial, bladder, and lung cancers. The most frequent hotspot mutations in PIK3CA are E542K, E545K, H1047R, and H1047L, and they account for 80-90% of all PIK3CA mutations detected in human malignancies. These PIK3CA mutations lead to increased catalytic activity of p110a, which causes downstream effects such as unregulated cell growth, proliferation, and survival.

Mutations in RAS proteins are found in over 20% of all human cancers. RAS proteins function as molecular switches that cycle between an active, GTP-bound state and an inactive, GDP-bound state. In the active state, RAS proteins interact with various effector proteins including PI3K, RAF kinase, and RalGDS, leading to activation of multiple downstream signaling pathways. Oncogenic RAS mutations are predominantly found at amino acid positions G12, G13, and Q61, and these mutations impair GTPase activities leading to the accumulation of active RAS proteins. The most common oncogenic RAS mutations are G12C, G12D, G12S, G12V, G12R, G13D, and Q61H.

RAS signaling through PI3K is necessary for normal lymphatic development and RAS-induced transformation, especially in lung cancer, where the interaction between mutant RAS and p110a-RBD is essential for tumor initiation and maintenance. RAS interactions with p110α-RBD have been shown to be crucial for epidermal growth factor (EGF) signaling to PI3K. Recent studies have shown that disrupting the RAS-PI3K interaction inhibits AKT and RAC1 activation in EGFR-mutant lung cancer cells, leading to reduced growth and survival and inhibiting EGFR-mutant-induced tumor onset. These results suggest that the binding of p110α to endogenous RAS proteins in EGFR-driven lung adenocarcinoma is critical in tumors driven by upstream activators of the RAS pathways and not just those in which RAS is mutationally activated.

Small GTPases (e.g., other than RAS) are also expected to bind the RBD of PI3Kα resulting in activation of signaling. The small GTPases Rac1 and CDC42 have been shown to bind the RBD of PI3Kβ and are hypothesized to also be capable of binding the RBD of PI3Kα. Accordingly, in some embodiments, the present disclosure encompasses the recognition that disrupting an interaction between PI3Kα and any small GTPase that binds the RBD of PI3Kα may be a useful therapeutic strategy for treating cancers and other indications. In some embodiments, a small GTPase is selected from Rac1, CDC42, and RAS proteins (including HRAS, NRAS, KRAS, RRAS, RRAS2, MRAS, and RIT1).

The frequency of oncogenic PIK3CA hotspot mutations across cancers has fueled the development and testing of numerous PI3K (e.g., PI3Kα) inhibitors. Most PI3K inhibitors that have entered clinical development thus far are reversible, ATP-competitive kinase inhibitors. Despite considerable efforts, the clinical outcome of PI3K inhibitor-based treatments for solid tumors has been disappointing, mainly due to intolerable toxicity and drug resistance. In 2019, the U.S. Food and Drug Administration (FDA) approved alpelisib (BYL719; Novartis Pharma AG), an inhibitor specific to the PI3Kα isoform, combined with fulvestrant for the treatment of patients diagnosed with HR+/HER2− PIK3CA-mutation. The therapeutic window of PI3K inhibitors is mainly limited by isoform selectivity and off-tumor toxicity. Moreover, hyperglycemia and hyperinsulinemia have been observed as major dose-limiting toxicities for p110α inhibitors, which prevent the use of sufficiently high doses to fully suppress PI3Kα signaling in the tumor. Hyperglycemia and hyperinsulinemia are considered on-target effects of PI3Kα inhibition, as inhibition of the PI3K/AKT pathway reduces glucose uptake, which in turn leads to increased secretion of insulin and subsequent activation of insulin/insulin-like growth factor I receptor in tumor cells, providing a survival mechanism for tumor cells and limiting the therapeutic efficacy of the PI3Kα inhibitor. Indeed, hyperglycemia was observed in 65% of patients in a Phase III clinical trial of alpelisib, leading to significant dose interruptions.

To overcome the limitations of current PI3Kα inhibitors, novel strategies to target PI3Kα need to be explored. Previous studies have suggested that inhibiting the RAS-p110α(RBD) interaction has minimal toxicity in adult animals while effectively causing tumor regression. The present disclosure encompasses the recognition that this therapeutic approach may be effective in various cancers including RAS-mutant-driven cancers and/or those driven by mutations or amplification of receptor tyrosine kinases (RTKs). The present disclosure also appreciates that this therapeutic modality may provide certain advantages over known PI3Kα inhibitors (e.g., those that target the ATP binding pocket of PI3Kα). For example, provided technologies may avoid hyperglycemia and insulin-driven resistance common to PI3Kα inhibitors, e.g., because such technologies target activation of PI3Kα by RAS, which is mostly present in transformed cells.

### Provided Compounds

In some embodiments, the present disclosure provides compounds useful for disrupting, inhibiting, and/or preventing the interaction between small GTPases (e.g., RAS proteins, as described herein) and PI3Kα proteins. In some embodiments, the present disclosure provides compounds capable of binding PI3Kα, such that (i) the interaction between the small GTPase (e.g., RAS protein, as described herein) and PI3Kα is disrupted, inhibited, or prevented; and/or (ii) the kinase activity of PI3Kα is not significantly inhibited. In some embodiments, such compounds bind PI3Kα reversibly. As used herein, a compound that binds “reversibly” refers to a compound that is able to bind to and become dissociated from a target protein kinase (e.g., PI3Kα). Often, but not always, reversible inhibitors are not able to form a covalent bond with a target protein kinase. In some embodiments, such compounds bind PI3Kα irreversibly. As used herein, a compound that binds “irreversibly” refers to a compound that is able to interact (e.g., to form a covalent bond) with a target protein kinase (e.g., PI3Kα) in a substantially non-reversible manner. In some embodiments, a reversible or irreversible inhibitor may be capable of interacting covalently with PI3Kα. For example, in some embodiments the present disclosure provides compounds comprising an electrophilic moiety (e.g., a Michael acceptor or the like) capable of binding (e.g., reversibly or irreversibly) to, e.g., a cysteine residue in the catalytic subunit of PI3Kα (e.g., C242). In some embodiments, provided compounds that interact with PI3Kα covalently are compounds of Formula I wherein -L-W comprises an electrophilic moiety (e.g., a Michael acceptor or the like) capable of binding (e.g., reversibly or irreversibly) to, e.g., a cysteine residue of PI3Kα (e.g., C242).

In some embodiments, the present disclosure provides a compound of formula I:

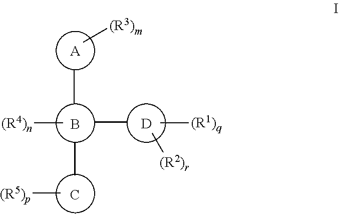

or a salt (e.g., pharmaceutically acceptable salt), ester, tautomer, prodrug, zwitterionic form, or stereoisomer thereof, wherein:
Ring A is selected from phenyl and a 5- to 6-membered heteroaryl ring having 1-3 heteroatoms independently selected from nitrogen, oxygen, and sulfur;
Ring B is selected from phenyl, a 6-membered heteroaryl ring having 1-2 nitrogen atoms, and a 9- to 10-membered bicyclic ring that comprises at least one 5- or 6-membered heteroaryl ring;
Ring C is selected from phenyl; a 5- to 6-membered heteroaryl ring having 1-3 heteroatoms independently selected from nitrogen, oxygen, and sulfur; a 5- to 8-membered bicyclic carbocyclic ring; a 4- to 8-membered heterocyclic ring having 1-2 heteroatoms independently selected from nitrogen, oxygen, and sulfur; and a 7- to 10-membered spirofused heterocyclic ring having 1-3 heteroatoms independently selected from nitrogen, oxygen, and sulfur, wherein each of the phenyl, heteroaryl, and heterocyclic rings is optionally fused to Ring E;
Ring D is selected from phenyl and a 5- to 6-membered heteroaryl ring having 1-3 heteroatoms independently selected from nitrogen, oxygen, and sulfur, wherein each of the phenyl and heteroaryl rings is optionally fused to Ring F;
Ring E is selected from a 5- to 6-membered carbocyclic ring; a 5- to 7-membered heterocyclic ring having 1-2 heteroatoms independently selected from nitrogen, oxygen, and sulfur; 7- to 10-membered spirofused heterocyclic ring having 1-3 heteroatoms independently selected from nitrogen, oxygen, and sulfur; and a 5- to 6-membered heteroaryl ring having 1-3 heteroatoms independently selected from nitrogen, oxygen, and sulfur, wherein Ring E is substituted by s instances of R^{5′};
Ring F is selected from phenyl; a 5- to 6-membered carbocyclic ring; a 5- to 7-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring F is substituted by u instances of -L-W and y instances of R^{2′};
R¹ is selected from -L-W, Ring D′, or a bivalent C₁₋₆ aliphatic chain substituted with Ring D′; each -L-W is —CN, or:
   each L is independently a bivalent straight or branched C₁₋₈ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—;
   each W is independently hydrogen, halogen, —CN, or an optionally substituted 3-10 membered monocyclic or bicyclic, saturated, partially unsaturated, or aryl ring having 0-3 heteroatoms independently selected from nitrogen, oxygen, or sulfur;
   each X is independently halogen, —OR, or —CN;
each Ring D′ is independently a 4- to 6-membered carbocyclic ring or a 4- to 6-membered heterocyclic ring having 1-2 heteroatoms independently selected from nitrogen, oxygen, and sulfur, wherein Ring D′ is substituted with t instances of -L-W;
each R² and R^{2′} is independently selected from oxo, halogen, —CN, —OR, and C₁₋₆ alkyl;
each R³ is independently selected from oxo, halogen, —CN, —OR, —O(CH₂)ᵥCy, —OCH₂CH₂OR, and optionally substituted C₁₋₆ aliphatic;
each Cy is independently a 5- to 6-membered heteroaryl ring having 1-3 heteroatoms independently selected from nitrogen, oxygen, and sulfur; a 3- to 6-membered carbocyclic ring; or a 4- to 6-membered heterocyclic ring having 1-2 heteroatoms independently selected from nitrogen, oxygen, and sulfur, wherein Cy is substituted with 0-2 instances of R⁶;
each R⁴ is independently selected from halogen and optionally substituted C₁₋₆ aliphatic;
each of R⁵ and R^{5′} is independently selected from oxo, ═NH, —CN, halogen, —OR, —N(R)₂, —SR, —C(O)R, —N(R)C(O)R, —(CH₂)ₓC(O)N(R)₂, —C(O)N(R)₂, —C(O)N(R)(CH₂)ₓCy, —(CH₂)ₓC(O)Cy, —OC(O)R, —C(O)OR, —SO₂R, —N(R)SO₂R, —N═S(O)(R)₂, —SO₂N(R)₂, —P(O)R₂, —(CH₂)ₓCy, —O(CH₂)ₓCy, and optionally substituted C₁₋₆ aliphatic;
each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
each R is independently hydrogen or an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
m is 0, 1, 2, or 3;
n is 0, 1, or 2;
p is 0, 1, 2, or 3;
q is 0 or 1;
r is 0, 1, or 2;
s is 0, 1, 2, or 3;
t is 0, 1, or 2;
u is 0 or 1;
each v is independently 0, 1, or 2;
each x is independently 0, 1, or 2; and
y is 0, 1, or 2.

In some embodiments, the present disclosure provides a compound of formula I, or a pharmaceutically acceptable salt thereof.

In some embodiments, the present disclosure provides a compound of formula I, or a salt (e.g., pharmaceutically acceptable salt), ester, tautomer, prodrug, zwitterionic form, or stereoisomer thereof, wherein:
Ring A is selected from phenyl and a 5- to 6-membered heteroaryl ring having 1-3 heteroatoms independently selected from nitrogen, oxygen, and sulfur;
Ring B is selected from phenyl, a 6-membered heteroaryl ring having 1-2 nitrogen atoms, and a 9- to 10-membered bicyclic ring that comprises at least one 5- or 6-membered heteroaryl ring;
Ring C is selected from phenyl; a 5- to 6-membered heteroaryl ring having 1-3 heteroatoms independently selected from nitrogen, oxygen, and sulfur; a 5- to 6-membered heterocyclic ring having 1-2 heteroatoms independently selected from nitrogen, oxygen, and sulfur; and a 9- to 10-membered spirofused heterocyclic ring having 1-3 heteroatoms independently selected from nitrogen, oxygen, and sulfur, wherein each of the phenyl, heteroaryl, and heterocyclic rings is optionally fused to Ring E;
Ring D is selected from phenyl and a 5- to 6-membered heteroaryl ring having 1-3 heteroatoms independently selected from nitrogen, oxygen, and sulfur, wherein each of the phenyl and heteroaryl rings is optionally fused to Ring F;
Ring E is selected from a 5- to 6-membered carbocyclic ring; a 5- to 7-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring E is substituted by s instances of R^{5′};
Ring F is selected from phenyl; a 5- to 6-membered carbocyclic ring; a 5- to 6-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring F is substituted by u instances of -L-W;
R¹ is selected from -L-W, Ring D′, or a bivalent C₁₋₆ aliphatic chain substituted with Ring D′;
   each L is independently a bivalent straight or branched C₁₋₈ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—;
   each W is independently hydrogen, halogen, —CN, or an optionally substituted 3-10 membered monocyclic or bicyclic, saturated, partially unsaturated, or aryl ring having 0-3 heteroatoms independently selected from nitrogen, oxygen, or sulfur;
   each X is independently halogen or —CN;
each Ring D′ is independently a 4- to 6-membered carbocyclic ring or a 4- to 6-membered heterocyclic ring having 1-2 heteroatoms independently selected from nitrogen, oxygen, and sulfur, wherein Ring D′ is substituted with t instances of -L-W;
each R² is independently selected from oxo, halogen, —CN, —OR, and C₁₋₆ alkyl;
each R³ is independently selected from oxo, halogen, —CN, —OR, —O(CH₂)ᵥCy, —OCH₂CH₂OR, and optionally substituted C₁₋₆ aliphatic;
each Cy is independently a 5- to 6-membered heteroaryl ring having 1-3 heteroatoms independently selected from nitrogen, oxygen, and sulfur; a 3- to 6-membered carbocyclic ring; or a 4- to 6-membered heterocyclic ring having 1-2 heteroatoms independently selected from nitrogen, oxygen, and sulfur, wherein Cy is substituted with 0-2 instances of R⁶;
each R⁴ is independently selected from halogen and optionally substituted C₁₋₆ aliphatic;
each of R⁵ and R^{5′} is independently selected from oxo, ═NH, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, —(CH₂)ₓCy, and optionally substituted C₁₋₆ aliphatic;
each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
each R is independently hydrogen or an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
m is 0, 1, 2, or 3;
n is 0, 1, or 2;
p is 0, 1, or 2;
q is 0 or 1;
r is 0, 1, or 2;
s is 0, 1, or 2;
t is 0, 1, or 2;
u is 0 or 1;
each v is independently 0, 1, or 2; and
each x is independently 0, 1, or 2.

In some embodiments, the present disclosure provides a compound of formula I, or a salt (e.g., pharmaceutically acceptable salt), ester, tautomer, prodrug, zwitterionic form, or stereoisomer thereof, wherein:
Ring A is phenyl;
Ring B is selected from a 9- to 10-membered bicyclic ring that comprises at least one 5- or 6-membered heteroaryl ring;
Ring C is selected from phenyl; a 5- to 6-membered heteroaryl ring having 1-3 heteroatoms independently selected from nitrogen, oxygen, and sulfur; a 5- to 8-membered bicyclic carbocyclic ring; a 4- to 8-membered heterocyclic ring having 1-2 heteroatoms independently selected from nitrogen, oxygen, and sulfur; and a 7- to 10-membered spirofused heterocyclic ring having 1-3 heteroatoms independently selected from nitrogen, oxygen, and sulfur, wherein each of the phenyl, heteroaryl, and heterocyclic rings is optionally fused to Ring E;
Ring D is selected from phenyl and a 5- to 6-membered heteroaryl ring having 1-3 heteroatoms independently selected from nitrogen, oxygen, and sulfur, wherein each of the phenyl and heteroaryl rings is optionally fused to Ring F;
Ring E is selected from a 5- to 6-membered carbocyclic ring; a 5- to 7-membered heterocyclic ring having 1-2 heteroatoms independently selected from nitrogen, oxygen, and sulfur; 7- to 10-membered spirofused heterocyclic ring having 1-3 heteroatoms independently selected from nitrogen, oxygen, and sulfur; and a 5- to 6-membered heteroaryl ring having 1-3 heteroatoms independently selected from nitrogen, oxygen, and sulfur, wherein Ring E is substituted by s instances of R^{5′};
Ring F is selected from phenyl; a 5- to 6-membered carbocyclic ring; a 5- to 7-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring F is substituted by u instances of -L-W and y instances of R^{2′};
R¹ is selected from -L-W, Ring D′, or a bivalent C₁₋₆ aliphatic chain substituted with Ring D′; each -L-W is —CN, or:
   each L is independently a bivalent straight or branched C₁₋₈ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—;
   each W is independently hydrogen, halogen, —CN, or an optionally substituted 3-10 membered monocyclic or bicyclic, saturated, partially unsaturated, or aryl ring having 0-3 heteroatoms independently selected from nitrogen, oxygen, or sulfur;
   each X is independently halogen, —OR, or —CN;
each Ring D′ is independently a 4- to 6-membered carbocyclic ring or a 4- to 6-membered heterocyclic ring having 1-2 heteroatoms independently selected from nitrogen, oxygen, and sulfur, wherein Ring D′ is substituted with t instances of -L-W;
each R² and R^{2′} is independently selected from oxo, halogen, —CN, —OR, and C₁₋₆ alkyl;
each R³ is independently selected from oxo, halogen, —CN, —OR, —O(CH₂)ᵥCy, —OCH₂CH₂OR, and optionally substituted C₁₋₆ aliphatic;
each Cy is independently a 5- to 6-membered heteroaryl ring having 1-3 heteroatoms independently selected from nitrogen, oxygen, and sulfur; a 3- to 6-membered carbocyclic ring; or a 4- to 6-membered heterocyclic ring having 1-2 heteroatoms independently selected from nitrogen, oxygen, and sulfur, wherein Cy is substituted with 0-2 instances of R⁶;
each R⁴ is independently selected from halogen and optionally substituted C₁₋₆ aliphatic;
each of R⁵ and R^{5′} is independently selected from oxo, ═NH, —CN, halogen, —OR, —N(R)₂, —SR, —C(O)R, —N(R)C(O)R, —(CH₂)ₓC(O)N(R)₂, —C(O)N(R)₂, —C(O)N(R)(CH₂)ₓCy, —(CH₂)ₓC(O)Cy, —OC(O)R, —C(O)OR, —SO₂R, —N(R)SO₂R, —N═S(O)(R)₂, —SO₂N(R)₂, —P(O)R₂, —(CH₂)ₓCy, —O(CH₂)ₓCy, and optionally substituted C₁₋₆ aliphatic;
each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
each R is independently hydrogen or an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
m is 2 or 3;
n is 0, 1, or 2;
p is 0, 1, 2, or 3;
q is 0 or 1;
r is 0, 1, or 2;
s is 0, 1, 2, or 3;
t is 0, 1, or 2;
u is 0 or 1;
each v is independently 0, 1, or 2;
each x is independently 0, 1, or 2; and
y is 0, 1, or 2.

In some embodiments, the present disclosure provides a compound of formula I, or a salt (e.g., pharmaceutically acceptable salt), ester, tautomer, prodrug, zwitterionic form, or stereoisomer thereof, wherein:
Ring A is phenyl;
Ring B is selected from a 9- to 10-membered bicyclic ring that comprises at least one 5- or 6-membered heteroaryl ring;
Ring C is selected from phenyl; a 5- to 6-membered heteroaryl ring having 1-3 heteroatoms independently selected from nitrogen, oxygen, and sulfur; a 5- to 8-membered bicyclic carbocyclic ring; a 4- to 8-membered heterocyclic ring having 1-2 heteroatoms independently selected from nitrogen, oxygen, and sulfur; and a 7- to 10-membered spirofused heterocyclic ring having 1-3 heteroatoms independently selected from nitrogen, oxygen, and sulfur, wherein each of the phenyl, heteroaryl, and heterocyclic rings is optionally fused to Ring E;
Ring D is selected from phenyl and a 5- to 6-membered heteroaryl ring having 1-3 heteroatoms independently selected from nitrogen, oxygen, and sulfur, wherein each of the phenyl and heteroaryl rings is fused to Ring F;
Ring E is selected from a 5- to 6-membered carbocyclic ring; a 5- to 7-membered heterocyclic ring having 1-2 heteroatoms independently selected from nitrogen, oxygen, and sulfur; 7- to 10-membered spirofused heterocyclic ring having 1-3 heteroatoms independently selected from nitrogen, oxygen, and sulfur; and a 5- to 6-membered heteroaryl ring having 1-3 heteroatoms independently selected from nitrogen, oxygen, and sulfur, wherein Ring E is substituted by s instances of R^{5′};
Ring F is selected from phenyl; a 5- to 6-membered carbocyclic ring; a 5- to 7-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring F is substituted by u instances of -L-W and y instances of R^{2′};
each -L-W is —CN, or:
   each L is independently a bivalent straight or branched C₁₋₈ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—;
   each W is independently hydrogen, halogen, —CN, or an optionally substituted 3-10 membered monocyclic or bicyclic, saturated, partially unsaturated, or aryl ring having 0-3 heteroatoms independently selected from nitrogen, oxygen, or sulfur;
   each X is independently halogen, —OR, or —CN;
each R² and R^{2′} is independently selected from oxo, halogen, —CN, —OR, and C₁₋₆ alkyl;
each R³ is independently selected from oxo, halogen, —CN, —OR, —O(CH₂)ᵥCy, —OCH₂CH₂OR, and optionally substituted C₁₋₆ aliphatic;
each Cy is independently a 5- to 6-membered heteroaryl ring having 1-3 heteroatoms independently selected from nitrogen, oxygen, and sulfur; a 3- to 6-membered carbocyclic ring; or a 4- to 6-membered heterocyclic ring having 1-2 heteroatoms independently selected from nitrogen, oxygen, and sulfur, wherein Cy is substituted with 0-2 instances of R⁶;
each R⁴ is independently selected from halogen and optionally substituted C₁₋₆ aliphatic;
each of R⁵ and R^{5′} is independently selected from oxo, ═NH, —CN, halogen, —OR, —N(R)₂, —SR, —C(O)R, —N(R)C(O)R, —(CH₂)ₓC(O)N(R)₂, —C(O)N(R)₂, —C(O)N(R)(CH₂)ₓCy, —(CH₂)ₓC(O)Cy, —OC(O)R, —C(O)OR, —SO₂R, —N(R)SO₂R, —N═S(O)(R)₂, —SO₂N(R)₂, —P(O)R₂, —(CH₂)ₓCy, —O(CH₂)ₓCy, and optionally substituted C₁₋₆ aliphatic;
each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
each R is independently hydrogen or an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
m is 2 or 3;
n is 0, 1, or 2;
p is 0, 1, 2, or 3;
q is 0;
r is 0, 1, or 2;
s is 0, 1, 2, or 3;
u is 0 or 1;
each v is independently 0, 1, or 2;
each x is independently 0, 1, or 2; and
y is 0, 1, or 2.

In some embodiments, the present disclosure provides a compound of formula I, or a salt (e.g., pharmaceutically acceptable salt), ester, tautomer, prodrug, zwitterionic form, or stereoisomer thereof, wherein:
Ring A is phenyl;
Ring B is selected from a 9- to 10-membered bicyclic ring that comprises at least one 5- or 6-membered heteroaryl ring;
Ring C is selected from phenyl; a 5- to 6-membered heteroaryl ring having 1-3 heteroatoms independently selected from nitrogen, oxygen, and sulfur; a 5- to 8-membered bicyclic carbocyclic ring; a 4- to 8-membered heterocyclic ring having 1-2 heteroatoms independently selected from nitrogen, oxygen, and sulfur; and a 7- to 10-membered spirofused heterocyclic ring having 1-3 heteroatoms independently selected from nitrogen, oxygen, and sulfur, wherein each of the phenyl, heteroaryl, and heterocyclic rings is fused to Ring E;
Ring D is selected from phenyl and a 5- to 6-membered heteroaryl ring having 1-3 heteroatoms independently selected from nitrogen, oxygen, and sulfur, wherein each of the phenyl and heteroaryl rings is fused to Ring F;
Ring E is selected from a 5- to 6-membered carbocyclic ring; a 5- to 7-membered heterocyclic ring having 1-2 heteroatoms independently selected from nitrogen, oxygen, and sulfur; 7- to 10-membered spirofused heterocyclic ring having 1-3 heteroatoms independently selected from nitrogen, oxygen, and sulfur; and a 5- to 6-membered heteroaryl ring having 1-3 heteroatoms independently selected from nitrogen, oxygen, and sulfur, wherein Ring E is substituted by s instances of R^{5′};
Ring F is selected from phenyl; a 5- to 6-membered carbocyclic ring; a 5- to 7-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring F is substituted by u instances of -L-W and y instances of R^{2′};
each -L-W is —CN, or:
   each L is independently a bivalent straight or branched C₁₋₈ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—;
   each W is independently hydrogen, halogen, —CN, or an optionally substituted 3-10 membered monocyclic or bicyclic, saturated, partially unsaturated, or aryl ring having 0-3 heteroatoms independently selected from nitrogen, oxygen, or sulfur;
   each X is independently halogen, —OR, or —CN;
each R² and R^{2′} is independently selected from oxo, halogen, —CN, —OR, and C₁₋₆ alkyl;
each R³ is independently selected from oxo, halogen, —CN, —OR, —O(CH₂)ᵥCy, —OCH₂CH₂OR, and optionally substituted C₁₋₆ aliphatic;
each Cy is independently a 5- to 6-membered heteroaryl ring having 1-3 heteroatoms independently selected from nitrogen, oxygen, and sulfur; a 3- to 6-membered carbocyclic ring; or a 4- to 6-membered heterocyclic ring having 1-2 heteroatoms independently selected from nitrogen, oxygen, and sulfur, wherein Cy is substituted with 0-2 instances of R⁶;
each R⁴ is independently selected from halogen and optionally substituted C₁₋₆ aliphatic;
each of R⁵ and R^{5′} is independently selected from oxo, ═NH, —CN, halogen, —OR, —N(R)₂, —SR, —C(O)R, —N(R)C(O)R, —(CH₂)ₓC(O)N(R)₂, —C(O)N(R)₂, —C(O)N(R)(CH₂)ₓCy, —(CH₂)ₓC(O)Cy, —OC(O)R, —C(O)OR, —SO₂R, —N(R)SO₂R, —N═S(O)(R)₂, —SO₂N(R)₂, —P(O)R₂, —(CH₂)ₓCy, —O(CH₂)ₓCy, and optionally substituted C₁₋₆ aliphatic;
each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
each R is independently hydrogen or an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
m is 2 or 3;
n is 0, 1, or 2;
p is 0, 1, 2, or 3;
q is 0;
r is 0, 1, or 2;
s is 0, 1, 2, or 3;
u is 0 or 1;
each v is independently 0, 1, or 2;
each x is independently 0, 1, or 2; and
y is 0, 1, or 2.

In some embodiments, the present disclosure provides a compound selected from formulae I-a, I-b, I-c, I-d, I-e, I-f, I-g, I-h I-i, I-j, and I-k:

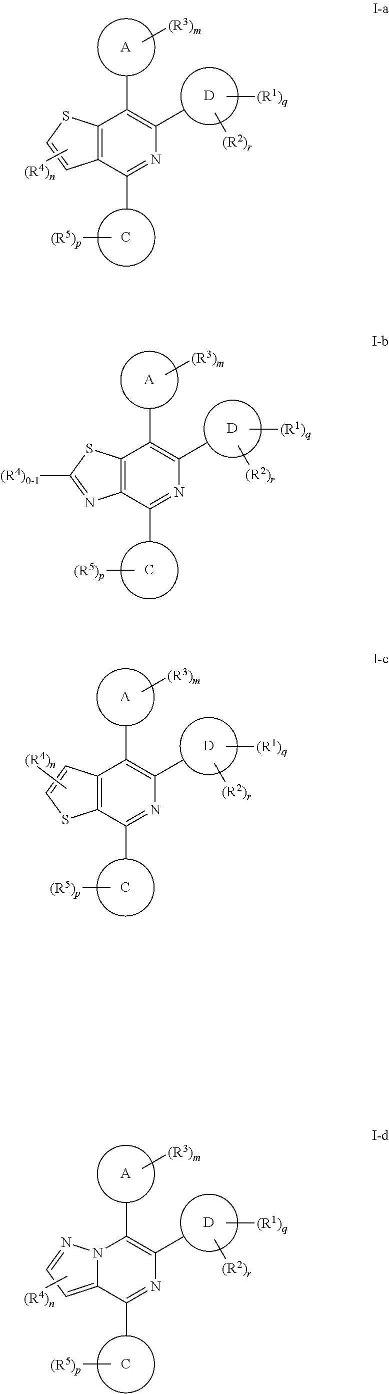

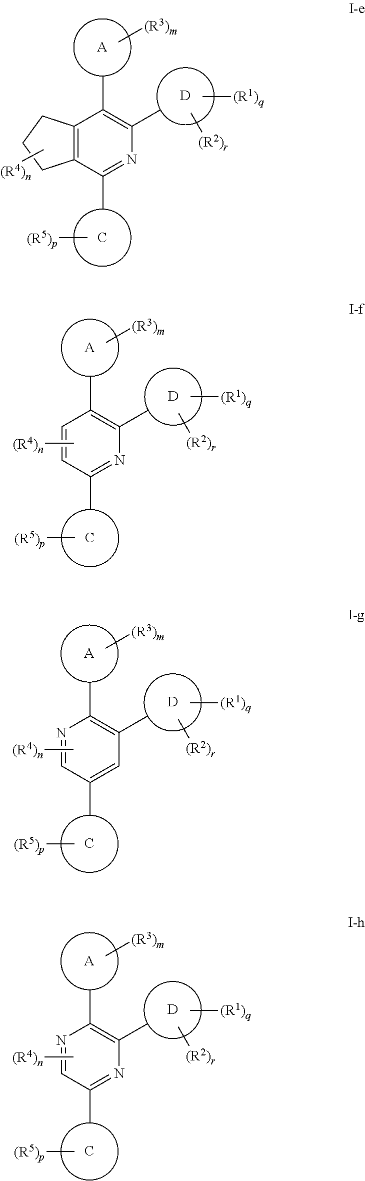

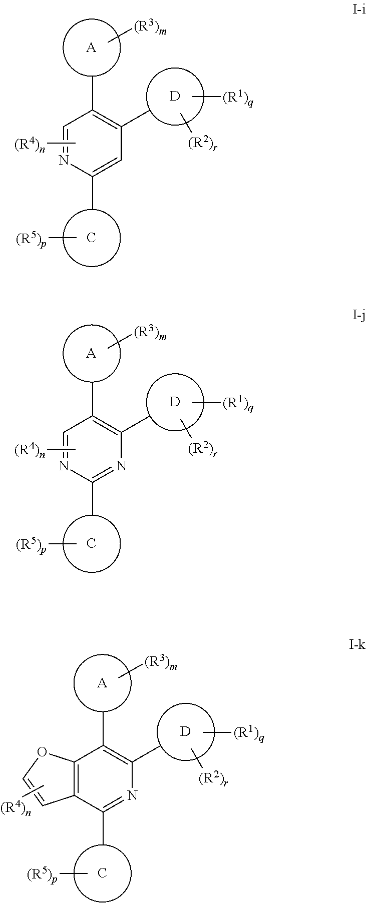

or a salt (e.g., pharmaceutically acceptable salt), ester, tautomer, prodrug, zwitterionic form, or stereoisomer thereof, wherein Ring A, Ring C, Ring D, R¹, R², R³, R⁴, R⁵, m, n, p, q, and r are as defined above for Formula I and described in classes and subclasses herein, both singly and in combination. In some embodiments, the present disclosure provides a compound of formula I-a or a pharmaceutically acceptable salt thereof. In some embodiments, the present disclosure provides a compound of formula I-b or a pharmaceutically acceptable salt thereof. In some embodiments, the present disclosure provides a compound of formula I-c or a pharmaceutically acceptable salt thereof. In some embodiments, the present disclosure provides a compound of formula I-d or a pharmaceutically acceptable salt thereof. In some embodiments, the present disclosure provides a compound of formula I-e or a pharmaceutically acceptable salt thereof. In some embodiments, the present disclosure provides a compound of formula I-f or a pharmaceutically acceptable salt thereof. In some embodiments, the present disclosure provides a compound of formula I-g or a pharmaceutically acceptable salt thereof. In some embodiments, the present disclosure provides a compound of formula I-h or a pharmaceutically acceptable salt thereof. In some embodiments, the present disclosure provides a compound of formula I-i or a pharmaceutically acceptable salt thereof. In some embodiments, the present disclosure provides a compound of formula I-j or a pharmaceutically acceptable salt thereof. In some embodiments, the present disclosure provides a compound of formula I-k or a pharmaceutically acceptable salt thereof.

In some embodiments, the present disclosure provides a compound selected from formulae I-a-i, I-b-i, I-c-i, I-d-i, I-e-i, I-f-i, I-g-i, I-h-i, I-i-i, I-j-i, and I-k-i.

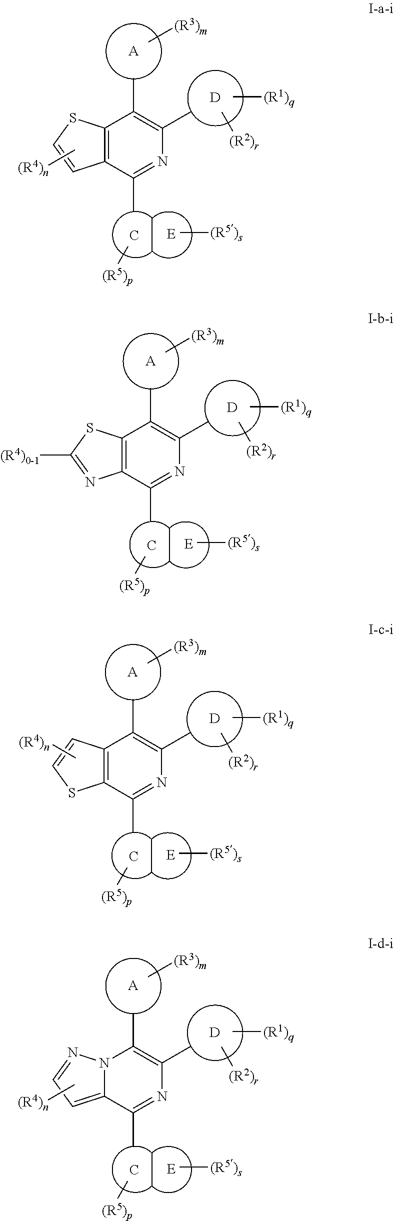

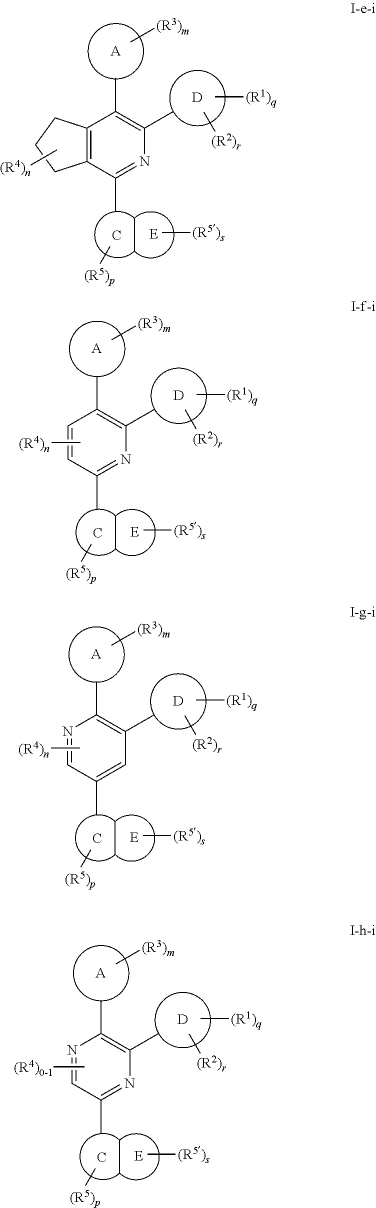

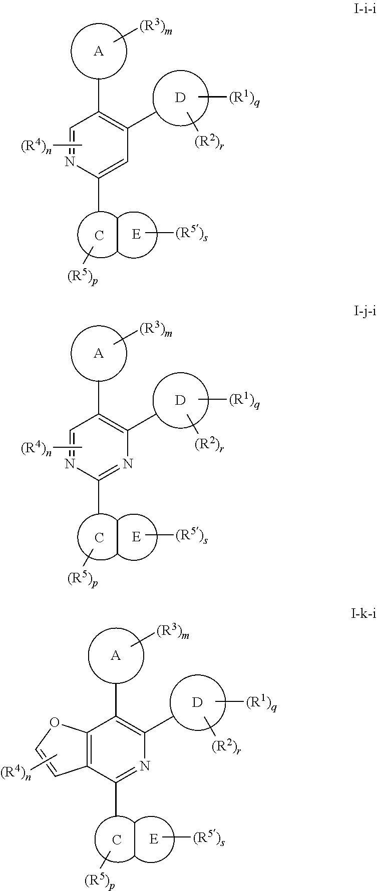

or a salt (e.g., pharmaceutically acceptable salt), ester, tautomer, prodrug, zwitterionic form, or stereoisomer thereof, wherein Ring A, Ring C, Ring D, Ring E, R¹, R², R³, R⁴, R⁵, R^{5′}, m, n, p, q, r, and s are as defined above for Formula I and described in classes and subclasses herein, both singly and in combination. In some embodiments, the present disclosure provides a compound of formula I-a-i or a pharmaceutically acceptable salt thereof. In some embodiments, the present disclosure provides a compound of formula I-b-i or a pharmaceutically acceptable salt thereof. In some embodiments, the present disclosure provides a compound of formula I-c-i or a pharmaceutically acceptable salt thereof. In some embodiments, the present disclosure provides a compound of formula I-d-i or a pharmaceutically acceptable salt thereof. In some embodiments, the present disclosure provides a compound of formula I-e-i or a pharmaceutically acceptable salt thereof. In some embodiments, the present disclosure provides a compound of formula I-f-i or a pharmaceutically acceptable salt thereof. In some embodiments, the present disclosure provides a compound of formula I-g-i or a pharmaceutically acceptable salt thereof. In some embodiments, the present disclosure provides a compound of formula I-h-i or a pharmaceutically acceptable salt thereof. In some embodiments, the present disclosure provides a compound of formula I-i-i or a pharmaceutically acceptable salt thereof. In some embodiments, the present disclosure provides a compound of formula I-j-i or a pharmaceutically acceptable salt thereof. In some embodiments, the present disclosure provides a compound of formula I-k-i or a pharmaceutically acceptable salt thereof.

In some embodiments, the present disclosure provides a compound selected from formulae I-a-ii, I-b-ii, I-c-ii, I-d-ii, I-e-ii, I-f-ii, I-g-ii, I-h-ii, I-i-ii, I-j-ii, and I-k-ii:

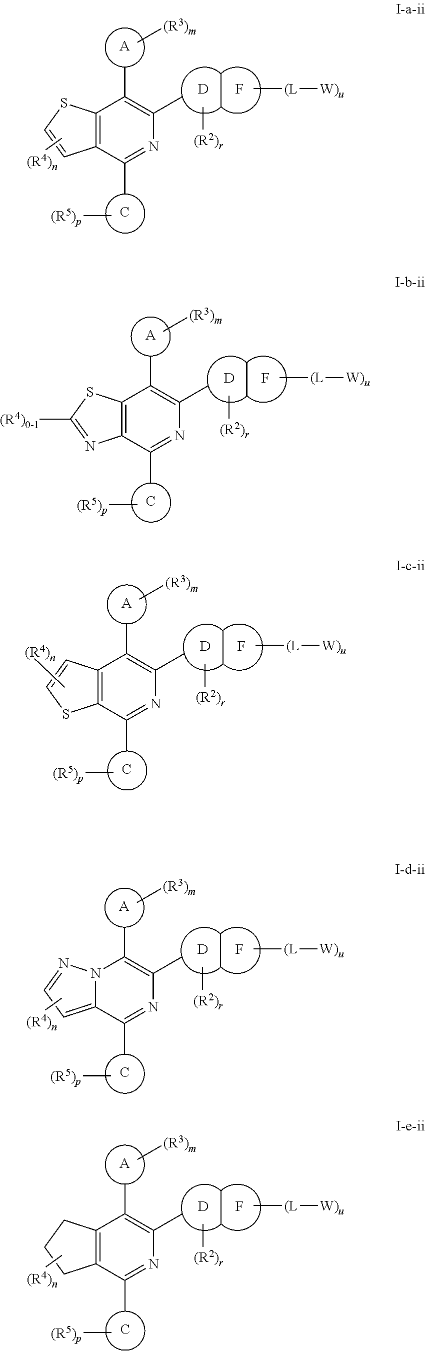

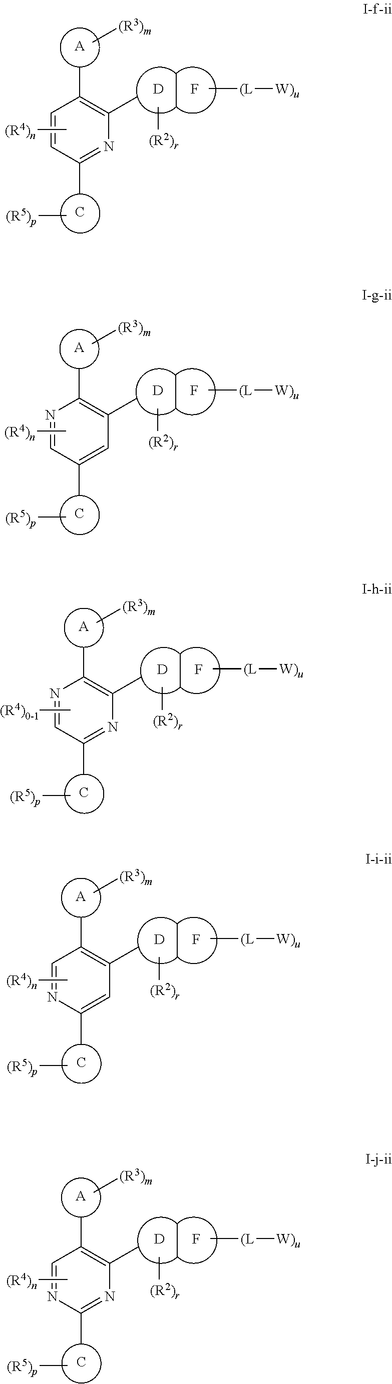

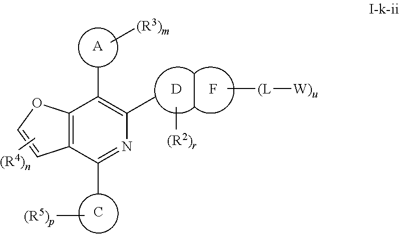

or a salt (e.g., pharmaceutically acceptable salt), ester, tautomer, prodrug, zwitterionic form, or stereoisomer thereof, wherein Ring A. Ring C, Ring D, Ring F, R², R³, R⁴, R⁵, L, W, m, n, p, r, and u are as defined above for Formula I and described in classes and subclasses herein, both singly and in combination. In some embodiments, the present disclosure provides a compound of formula I-a-ii or a pharmaceutically acceptable salt thereof. In some embodiments, the present disclosure provides a compound of formula I-b-ii or a pharmaceutically acceptable salt thereof. In some embodiments, the present disclosure provides a compound of formula I-c-ii or a pharmaceutically acceptable salt thereof. In some embodiments, the present disclosure provides a compound of formula I-d-ii or a pharmaceutically acceptable salt thereof. In some embodiments, the present disclosure provides a compound of formula I-e-ii or a pharmaceutically acceptable salt thereof. In some embodiments, the present disclosure provides a compound of formula I-f-ii or a pharmaceutically acceptable salt thereof. In some embodiments, the present disclosure provides a compound of formula I-g-ii or a pharmaceutically acceptable salt thereof. In some embodiments, the present disclosure provides a compound of formula I-h-ii or a pharmaceutically acceptable salt thereof. In some embodiments, the present disclosure provides a compound of formula I-i-ii or a pharmaceutically acceptable salt thereof. In some embodiments, the present disclosure provides a compound of formula I-j-ii or a pharmaceutically acceptable salt thereof. In some embodiments, the present disclosure provides a compound of formula I-k-ii or a pharmaceutically acceptable salt thereof.

In some embodiments, the present disclosure provides a compound selected from formulae I-a-ii′, I-b-ii′, I-c-ii′, I-d-ii′, I-e-ii′, I-f-ii′, I-g-ii′, I-h-ii′, I-i-ii′, I-j-ii′, and I-k-ii′:

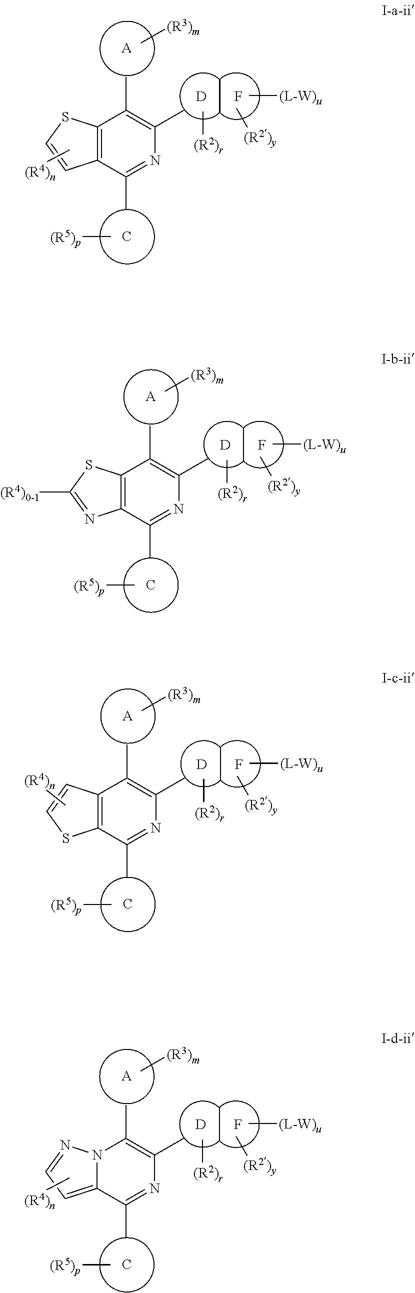

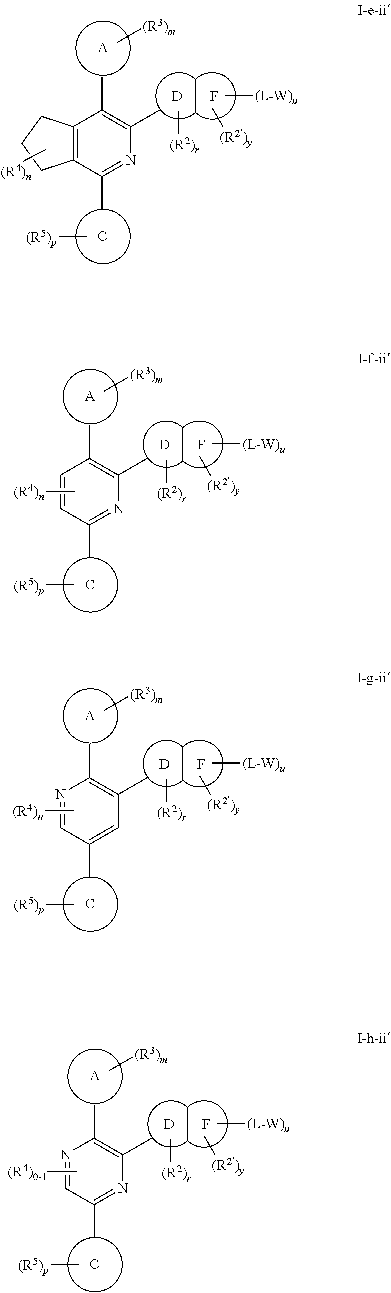

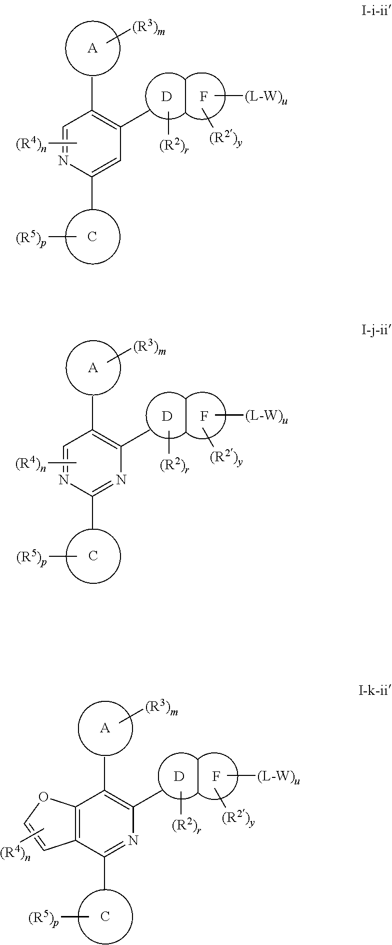

or a salt (e.g., pharmaceutically acceptable salt), ester, tautomer, prodrug, zwitterionic form, or stereoisomer thereof, wherein Ring A, Ring C, Ring D, Ring F, R², R^{2′}, R³, R⁴, R⁵, L, W, m, n, p, r, u, and y are as defined above for Formula I and described in classes and subclasses herein, both singly and in combination. In some embodiments, the present disclosure provides a compound of formula I-a-ii′ or a pharmaceutically acceptable salt thereof. In some embodiments, the present disclosure provides a compound of formula I-b-ii′ or a pharmaceutically acceptable salt thereof. In some embodiments, the present disclosure provides a compound of formula I-c-ii′ or a pharmaceutically acceptable salt thereof. In some embodiments, the present disclosure provides a compound of formula I-d-ii′ or a pharmaceutically acceptable salt thereof. In some embodiments, the present disclosure provides a compound of formula I-e-ii′ or a pharmaceutically acceptable salt thereof. In some embodiments, the present disclosure provides a compound of formula I-f-ii′ or a pharmaceutically acceptable salt thereof. In some embodiments, the present disclosure provides a compound of formula I-g-ii′ or a pharmaceutically acceptable salt thereof. In some embodiments, the present disclosure provides a compound of formula I-h-ii′ or a pharmaceutically acceptable salt thereof. In some embodiments, the present disclosure provides a compound of formula I-i-ii′ or a pharmaceutically acceptable salt thereof. In some embodiments, the present disclosure provides a compound of formula I-j-ii′ or a pharmaceutically acceptable salt thereof. In some embodiments, the present disclosure provides a compound of formula I-k-ii′ or a pharmaceutically acceptable salt thereof.

In some embodiments, the present disclosure provides a compound selected from formulae I-a-iii, I-b-iii, I-c-iii, I-d-iii, I-e-iii, I-f-iii, I-g-iii, I-h-iii, I-i-iii, J-j-iii, and I-k-iii:

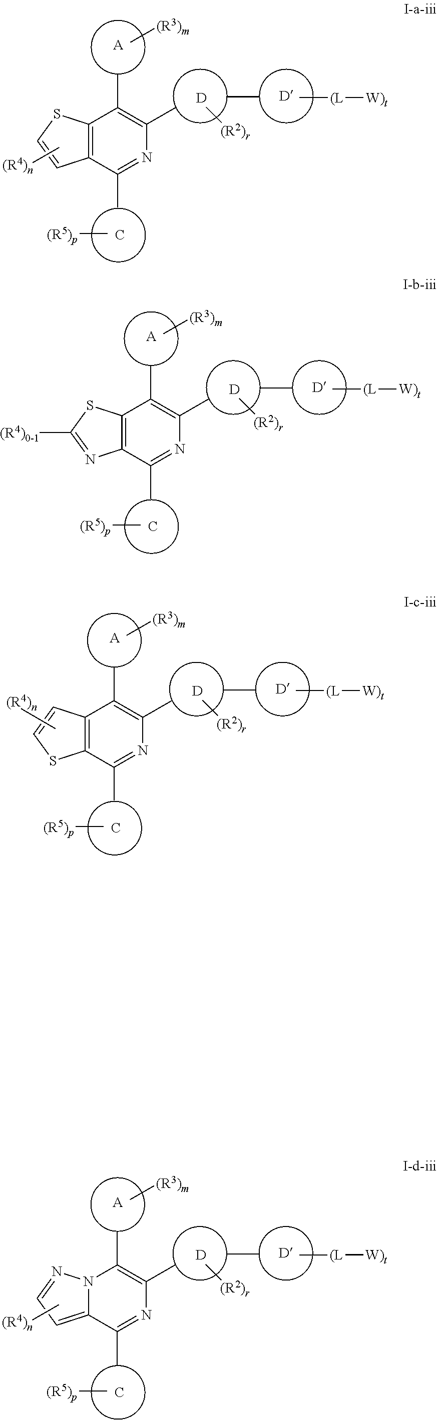

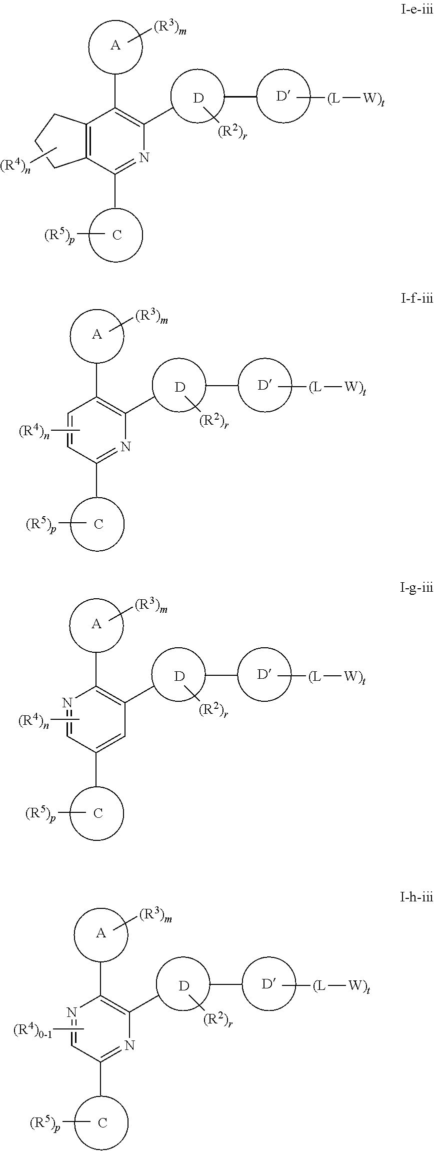

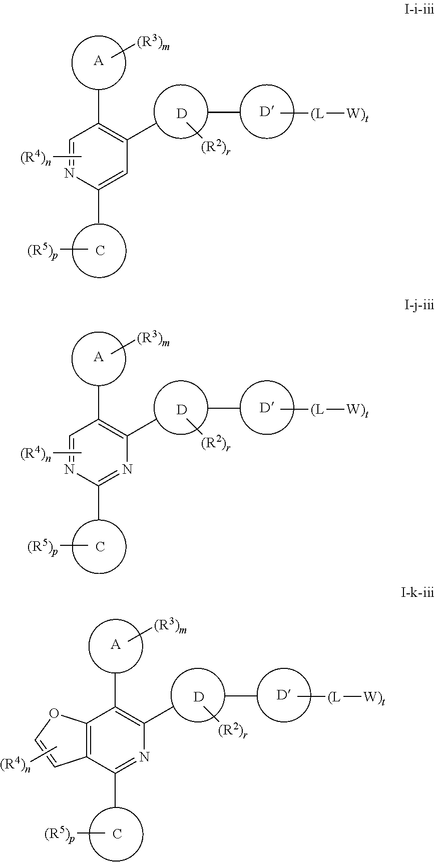

or a salt (e.g., pharmaceutically acceptable salt), ester, tautomer, prodrug, zwitterionic form, or stereoisomer thereof, wherein Ring A, Ring C, Ring D, Ring D′, L, R², R³, R⁴, R⁵, W, m, n, p, r, and t are as defined above for Formula I and described in classes and subclasses herein, both singly and in combination. In some embodiments, the present disclosure provides a compound of formula I-a-iii or a pharmaceutically acceptable salt thereof. In some embodiments, the present disclosure provides a compound of formula I-b-iii or a pharmaceutically acceptable salt thereof. In some embodiments, the present disclosure provides a compound of formula I-c-iii or a pharmaceutically acceptable salt thereof. In some embodiments, the present disclosure provides a compound of formula I-d-iii or a pharmaceutically acceptable salt thereof. In some embodiments, the present disclosure provides a compound of formula I-e-iii or a pharmaceutically acceptable salt thereof. In some embodiments, the present disclosure provides a compound of formula I-f-iii or a pharmaceutically acceptable salt thereof. In some embodiments, the present disclosure provides a compound of formula I-g-iii or a pharmaceutically acceptable salt thereof. In some embodiments, the present disclosure provides a compound of formula I-h-iii or a pharmaceutically acceptable salt thereof. In some embodiments, the present disclosure provides a compound of formula I-i-iii or a pharmaceutically acceptable salt thereof. In some embodiments, the present disclosure provides a compound of formula I-j-iii or a pharmaceutically acceptable salt thereof. In some embodiments, the present disclosure provides a compound of formula I-k-iii or a pharmaceutically acceptable salt thereof.

In some embodiments, the present disclosure provides a compound selected from formulae I-a-iv, I-b-iv, I-c-iv, I-d-iv, I-e-iv, I-f-iv, I-g-iv, I-h-iv, I-i-iv, I-j-iv, and I-k-iv:

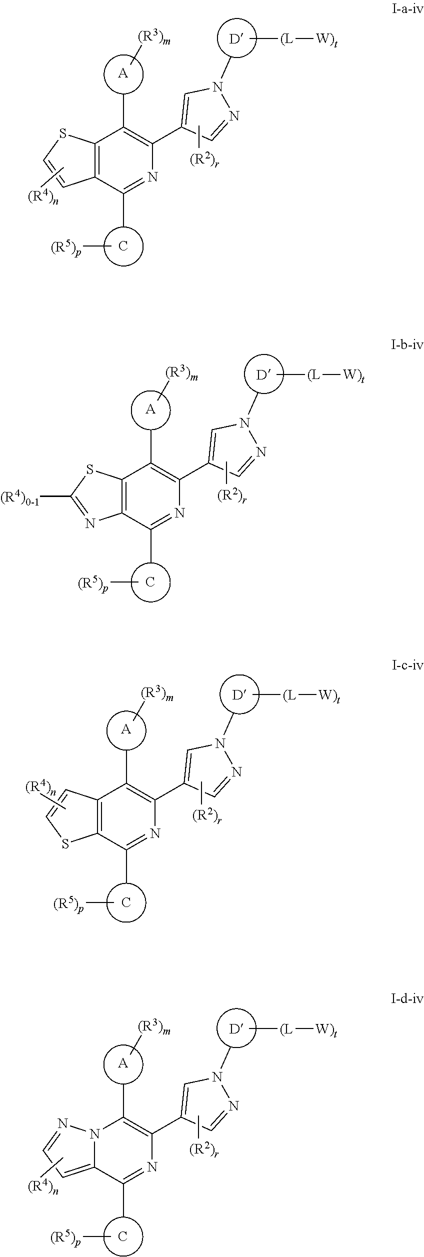

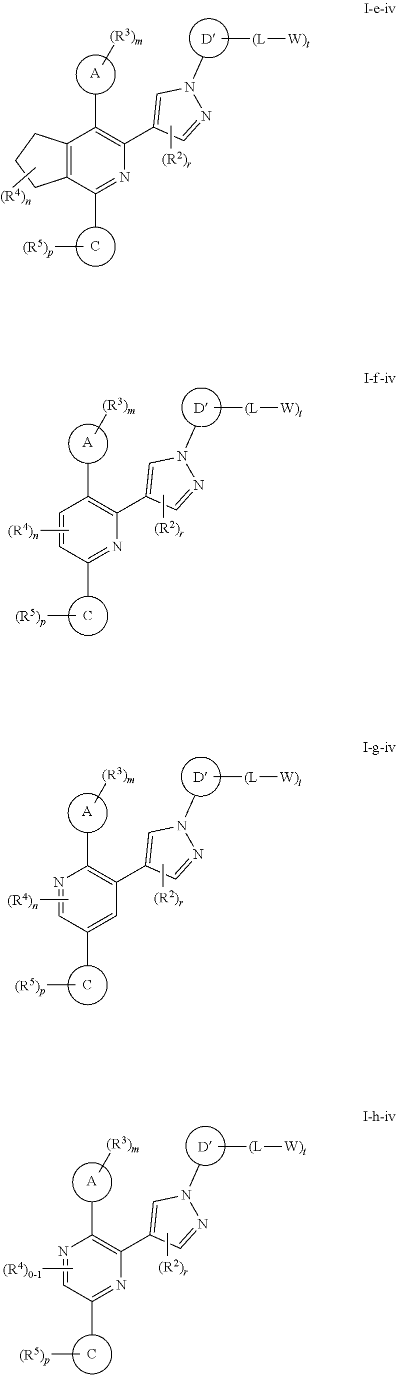

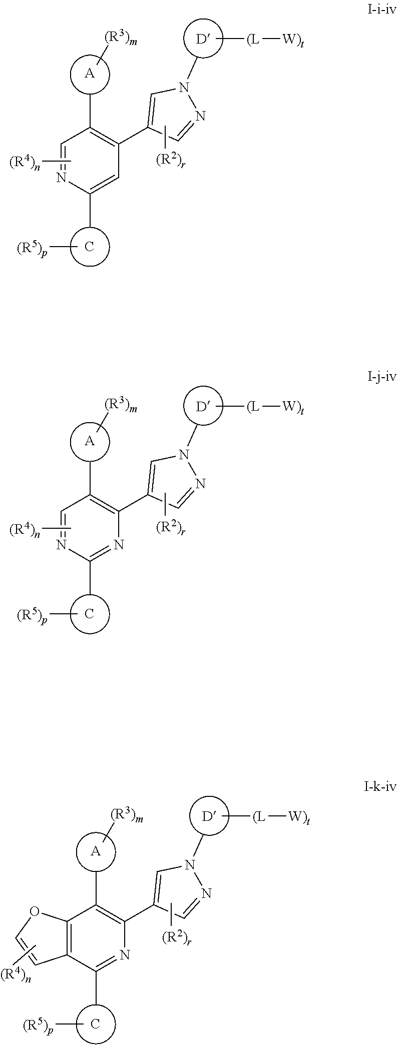

or a salt (e.g., pharmaceutically acceptable salt), ester, tautomer, prodrug, zwitterionic form, or stereoisomer thereof, wherein Ring A, Ring C, Ring D′, L, R², R³, R⁴, R⁵, W, m, n, p, r, and t are as defined above for Formula I and described in classes and subclasses herein, both singly and in combination. In some embodiments, the present disclosure provides a compound of formula I-a-iv or a pharmaceutically acceptable salt thereof. In some embodiments, the present disclosure provides a compound of formula I-b-iv or a pharmaceutically acceptable salt thereof. In some embodiments, the present disclosure provides a compound of formula I-c-iv or a pharmaceutically acceptable salt thereof. In some embodiments, the present disclosure provides a compound of formula I-d-iv or a pharmaceutically acceptable salt thereof. In some embodiments, the present disclosure provides a compound of formula I-e-iv or a pharmaceutically acceptable salt thereof. In some embodiments, the present disclosure provides a compound of formula I-f-iv or a pharmaceutically acceptable salt thereof. In some embodiments, the present disclosure provides a compound of formula I-g-iv or a pharmaceutically acceptable salt thereof. In some embodiments, the present disclosure provides a compound of formula I-h-iv or a pharmaceutically acceptable salt thereof. In some embodiments, the present disclosure provides a compound of formula I-i-iv or a pharmaceutically acceptable salt thereof. In some embodiments, the present disclosure provides a compound of formula I-j-iv or a pharmaceutically acceptable salt thereof. In some embodiments, the present disclosure provides a compound of formula I-k-iv or a pharmaceutically acceptable salt thereof.

In some embodiments, the present disclosure provides a compound selected from formulae I-a-v, I-b-v, I-c-v, I-d-v, I-e-v, I-f-v, I-g-v, I-h-v, I-i-v, I-j-v, and I-k-v:

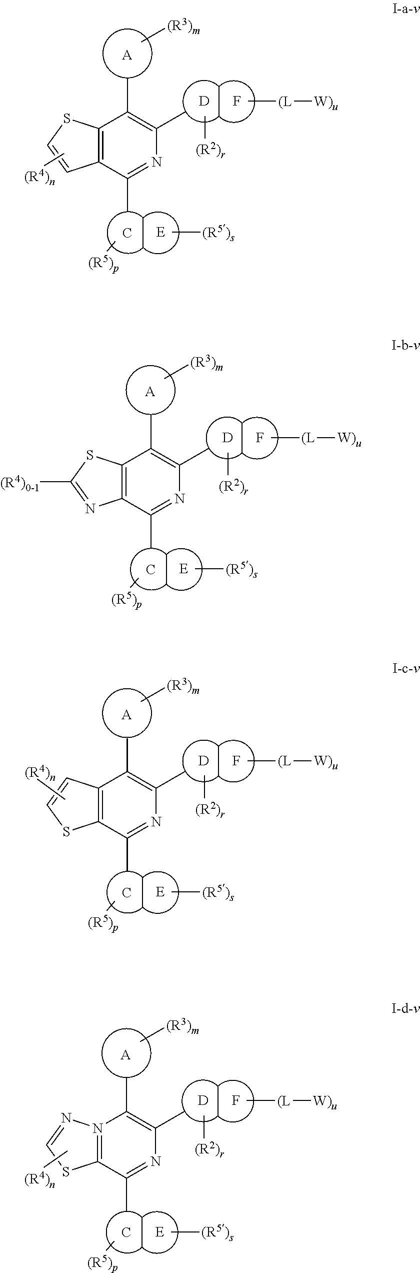

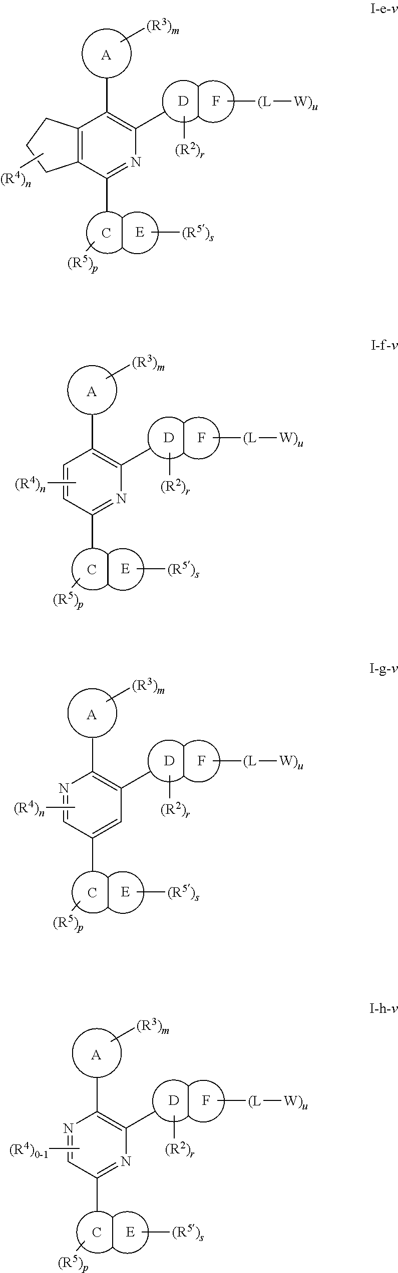

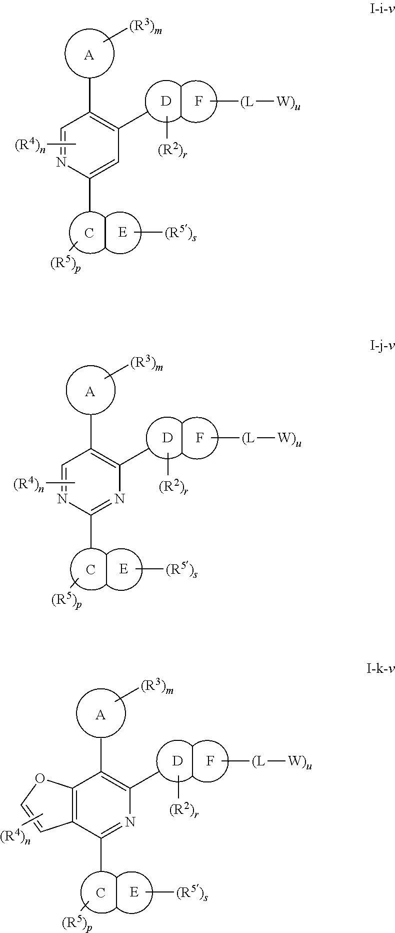

or a salt (e.g., pharmaceutically acceptable salt), ester, tautomer, prodrug, zwitterionic form, or stereoisomer thereof, wherein Ring A, Ring C, Ring D, Ring E, Ring F, R², R³, R⁴, R⁵, R^{5′}, L, W, m, n, p, r, s, and u are as defined above for Formula I and described in classes and subclasses herein, both singly and in combination. In some embodiments, the present disclosure provides a compound of formula I-a-v or a pharmaceutically acceptable salt thereof. In some embodiments, the present disclosure provides a compound of formula I-b-v or a pharmaceutically acceptable salt thereof. In some embodiments, the present disclosure provides a compound of formula I-c-v or a pharmaceutically acceptable salt thereof. In some embodiments, the present disclosure provides a compound of formula I-d-v or a pharmaceutically acceptable salt thereof. In some embodiments, the present disclosure provides a compound of formula I-e-v or a pharmaceutically acceptable salt thereof. In some embodiments, the present disclosure provides a compound of formula I-f-v or a pharmaceutically acceptable salt thereof. In some embodiments, the present disclosure provides a compound of formula I-g-v or a pharmaceutically acceptable salt thereof. In some embodiments, the present disclosure provides a compound of formula I-h-v or a pharmaceutically acceptable salt thereof. In some embodiments, the present disclosure provides a compound of formula I-i-v or a pharmaceutically acceptable salt thereof. In some embodiments, the present disclosure provides a compound of formula I-j-v or a pharmaceutically acceptable salt thereof. In some embodiments, the present disclosure provides a compound of formula I-k-v or a pharmaceutically acceptable salt thereof.

In some embodiments, the present disclosure provides a compound selected from formulae I-a-v′, I-b-v′, I-c-v′, I-d-v′, I-e-v′, I-f-v′, I-g-v′, I-h-v′, I-i-v′, I-j-v′, and I-k-v′:

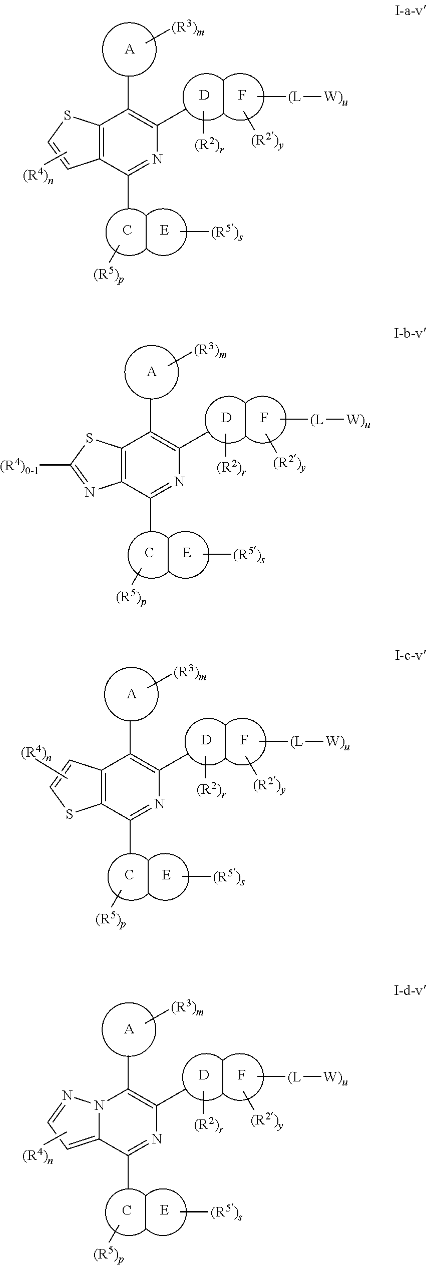

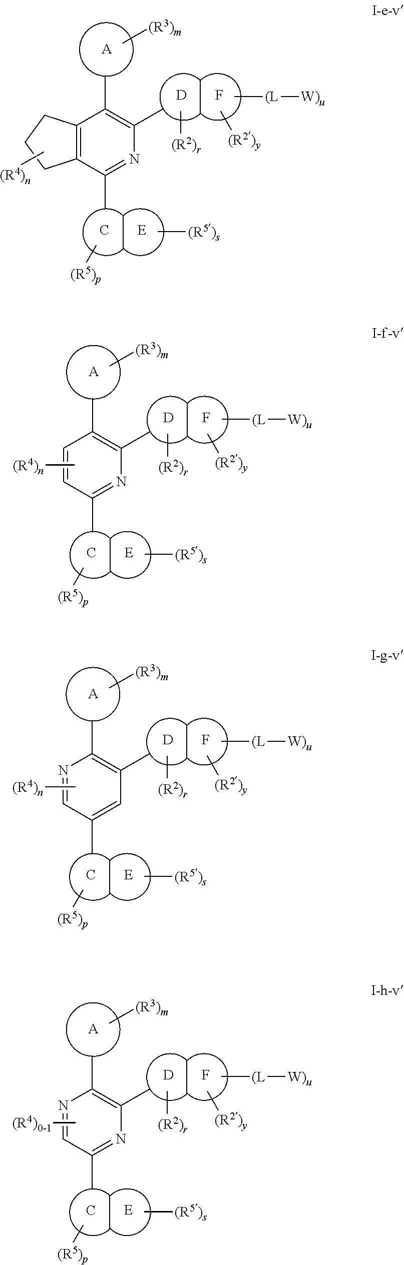

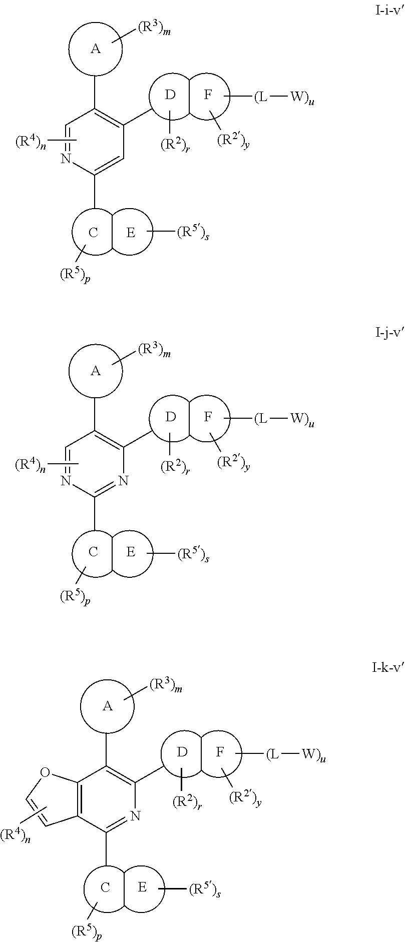

or a salt (e.g., pharmaceutically acceptable salt), ester, tautomer, prodrug, zwitterionic form, or stereoisomer thereof, wherein Ring A, Ring C, Ring D, Ring E, Ring F, R², R^{2′}, R³, R⁴, R⁵, R^{5′}, L, W, m, n, p, r, s, u, and y are as defined above for Formula I and described in classes and subclasses herein, both singly and in combination. In some embodiments, the present disclosure provides a compound of formula I-a-v′ or a pharmaceutically acceptable salt thereof. In some embodiments, the present disclosure provides a compound of formula I-b-v′ or a pharmaceutically acceptable salt thereof. In some embodiments, the present disclosure provides a compound of formula I-c-v′ or a pharmaceutically acceptable salt thereof. In some embodiments, the present disclosure provides a compound of formula I-d-v′ or a pharmaceutically acceptable salt thereof. In some embodiments, the present disclosure provides a compound of formula I-e-v′ or a pharmaceutically acceptable salt thereof. In some embodiments, the present disclosure provides a compound of formula I-f-v′ or a pharmaceutically acceptable salt thereof. In some embodiments, the present disclosure provides a compound of formula I-g-v′ or a pharmaceutically acceptable salt thereof. In some embodiments, the present disclosure provides a compound of formula I-h-v′ or a pharmaceutically acceptable salt thereof. In some embodiments, the present disclosure provides a compound of formula I-i-v′ or a pharmaceutically acceptable salt thereof. In some embodiments, the present disclosure provides a compound of formula I-j-v′ or a pharmaceutically acceptable salt thereof. In some embodiments, the present disclosure provides a compound of formula I-k-v′ or a pharmaceutically acceptable salt thereof.

In some embodiments, the present disclosure provides a compound selected from formulae I-l, I-m, I-n, I-p, I-q, and I-r:

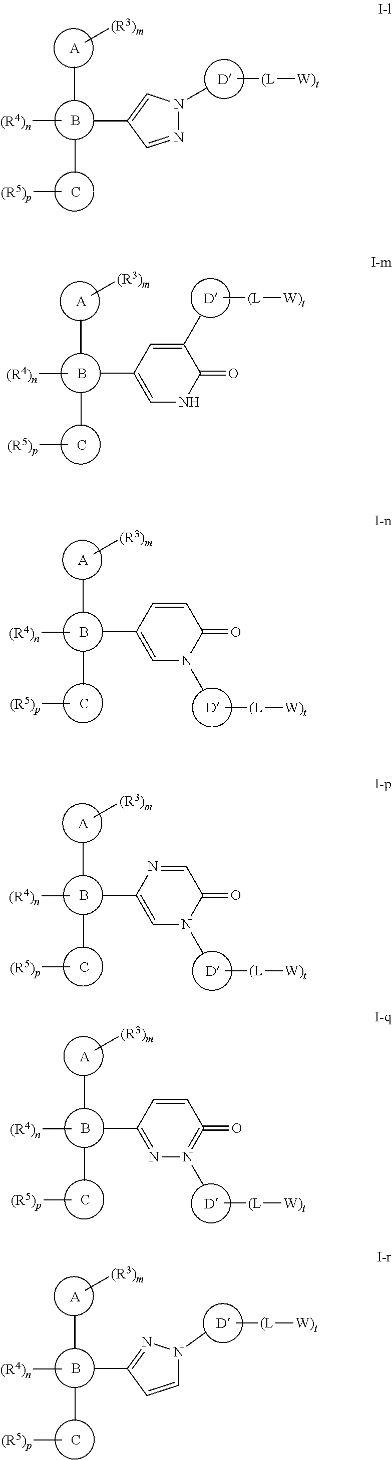

or a salt (e.g., pharmaceutically acceptable salt), ester, tautomer, prodrug, zwitterionic form, or stereoisomer thereof, wherein Ring A, Ring B, Ring C, Ring D′, L, R³, R⁴, R⁵, W, m, n, p, and t are as defined above for Formula I and described in classes and subclasses herein, both singly and in combination. In some embodiments, the present disclosure provides a compound of formula I-1 or a pharmaceutically acceptable salt thereof. In some embodiments, the present disclosure provides a compound of formula I-m or a pharmaceutically acceptable salt thereof. In some embodiments, the present disclosure provides a compound of formula I-n or a pharmaceutically acceptable salt thereof. In some embodiments, the present disclosure provides a compound of formula I-p or a pharmaceutically acceptable salt thereof. In some embodiments, the present disclosure provides a compound of formula I-q or a pharmaceutically acceptable salt thereof. In some embodiments, the present disclosure provides a compound of formula I-r or a pharmaceutically acceptable salt thereof.

In some embodiments, the present disclosure provides a compound of formula IA:

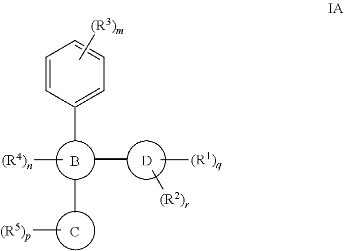

or a salt (e.g., pharmaceutically acceptable salt), ester, tautomer, prodrug, zwitterionic form, or stereoisomer thereof, wherein Ring B, Ring C, Ring D, R¹, R², R³, R⁴, R⁵, m, n, p, q, and r are as defined above for Formula I and described in classes and subclasses herein, both singly and in combination. In some embodiments, the present disclosure provides a compound of formula IA or a pharmaceutically acceptable salt thereof.

In some embodiments, the present disclosure provides a compound of formula IA1:

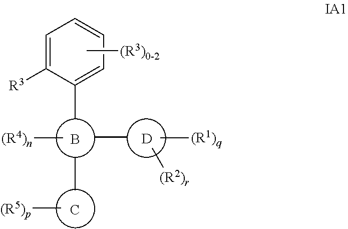

or a salt (e.g., pharmaceutically acceptable salt), ester, tautomer, prodrug, zwitterionic form, or stereoisomer thereof, wherein Ring B, Ring C, Ring D, R¹, R², R³, R⁴, R⁵, n, p, q, and r are as defined above for Formula I and described in classes and subclasses herein, both singly and in combination. In some embodiments, the present disclosure provides a compound of formula IA1 or a pharmaceutically acceptable salt thereof.

In some embodiments, the present disclosure provides a compound of formula IB:

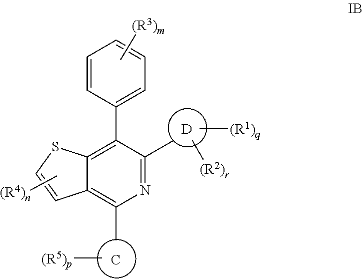

or a salt (e.g., pharmaceutically acceptable salt), ester, tautomer, prodrug, zwitterionic form, or stereoisomer thereof, wherein Ring C, Ring D, R¹, R², R³, R⁴, R⁵, m, n, p, q, and r are as defined above for Formula I and described in classes and subclasses herein, both singly and in combination. In some embodiments, the present disclosure provides a compound of formula IB or a pharmaceutically acceptable salt thereof.

In some embodiments, the present disclosure provides a compound of formula IB1:

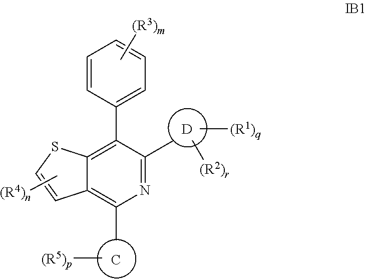

or a salt (e.g., pharmaceutically acceptable salt), ester, tautomer, prodrug, zwitterionic form, or stereoisomer thereof, wherein Ring C, Ring D, R¹, R², R³, R⁴, R⁵, n, p, q, and r are as defined above for Formula I and described in classes and subclasses herein, both singly and in combination. In some embodiments, the present disclosure provides a compound of formula IB1 or a pharmaceutically acceptable salt thereof.

In some embodiments, the present disclosure provides a compound of formula IC:

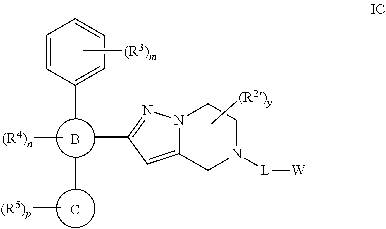

or a salt (e.g., pharmaceutically acceptable salt), ester, tautomer, prodrug, zwitterionic form, or stereoisomer thereof, wherein Ring B, Ring C, L, R^{2′}, R³, R⁴, R⁵, W, m, n, p, and y are as defined above for Formula I and described in classes and subclasses herein, both singly and in combination. In some embodiments, the present disclosure provides a compound of formula IC or a pharmaceutically acceptable salt thereof.

In some embodiments, the present disclosure provides a compound of formula IC1:

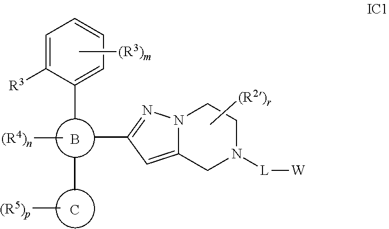

or a salt (e.g., pharmaceutically acceptable salt), ester, tautomer, prodrug, zwitterionic form, or stereoisomer thereof, wherein Ring B, Ring C, L, R^{2′}, R³, R⁴, R⁵, W, n, p, and y are as defined above for Formula I and described in classes and subclasses herein, both singly and in combination. In some embodiments, the present disclosure provides a compound of formula IC1 or a pharmaceutically acceptable salt thereof.

In some embodiments, the present disclosure provides a compound of formula ID:

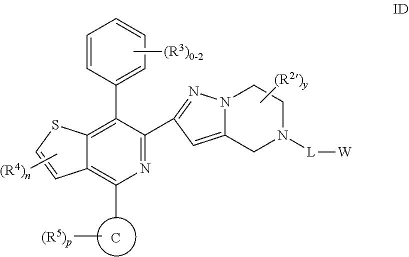

or a salt (e.g., pharmaceutically acceptable salt), ester, tautomer, prodrug, zwitterionic form, or stereoisomer thereof, wherein Ring C, L, R^{2′}, R³, R⁴, R⁵, W, m, n, p, and y are as defined above for Formula I and described in classes and subclasses herein, both singly and in combination. In some embodiments, the present disclosure provides a compound of formula ID or a pharmaceutically acceptable salt thereof.

In some embodiments, the present disclosure provides a compound of formula ID1:

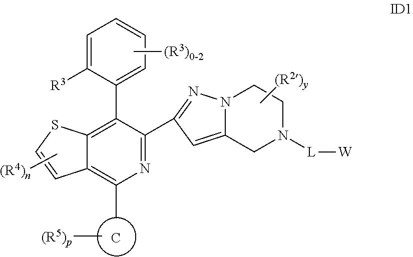

or a salt (e.g., pharmaceutically acceptable salt), ester, tautomer, prodrug, zwitterionic form, or stereoisomer thereof, wherein Ring C, L, R^{2′}, R³, R⁴, R⁵, W, n, p, and y are as defined above for Formula I and described in classes and subclasses herein, both singly and in combination. In some embodiments, the present disclosure provides a compound of formula ID1 or a pharmaceutically acceptable salt thereof.

In some embodiments, the present disclosure provides a compound of formula IE:

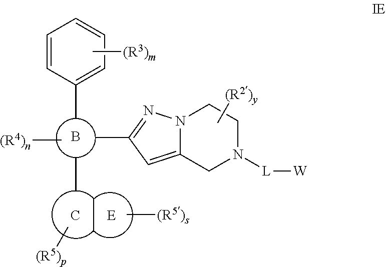

or a salt (e.g., pharmaceutically acceptable salt), ester, tautomer, prodrug, zwitterionic form, or stereoisomer thereof, wherein Ring B, Ring C. Ring E, L, R^{2′}, R³, R⁴, R⁵, R^{5′}, W, m, n, p, s, and y are as defined above for Formula I and described in classes and subclasses herein, both singly and in combination. In some embodiments, the present disclosure provides a compound of formula IE or a pharmaceutically acceptable salt thereof.

In some embodiments, the present disclosure provides a compound of formula IE1:

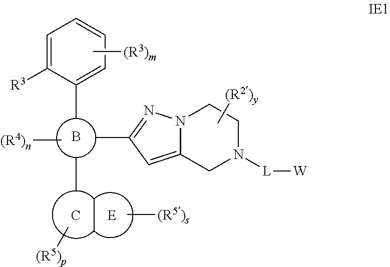

or a salt (e.g., pharmaceutically acceptable salt), ester, tautomer, prodrug, zwitterionic form, or stereoisomer thereof, wherein Ring B, Ring C, Ring E, L, R^{2′}, R³, R⁴, R⁵, R^{5′}, W, n, p, s, and y are as defined above for Formula I and described in classes and subclasses herein, both singly and in combination. In some embodiments, the present disclosure provides a compound of formula IE1 or a pharmaceutically acceptable salt thereof.

In some embodiments, the present disclosure provides a compound of formula IF:

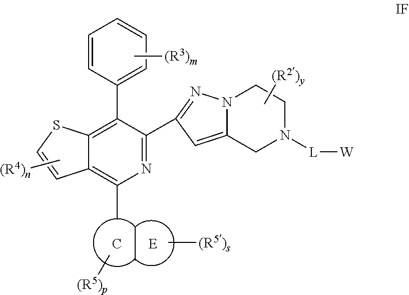

or a salt (e.g., pharmaceutically acceptable salt), ester, tautomer, prodrug, zwitterionic form, or stereoisomer thereof, wherein Ring C, Ring E, L, R^{2′}, R³, R⁴, R⁵, R^{5′}, W, m, n, p, s, and y are as defined above for Formula I and described in classes and subclasses herein, both singly and in combination. In some embodiments, the present disclosure provides a compound of formula IF or a pharmaceutically acceptable salt thereof.

In some embodiments, the present disclosure provides a compound of formula IF1:

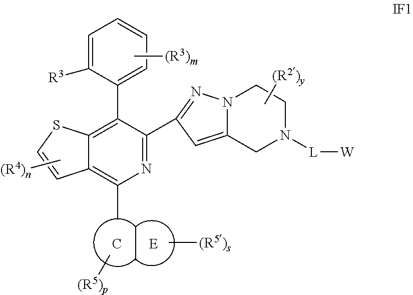

or a salt (e.g., pharmaceutically acceptable salt), ester, tautomer, prodrug, zwitterionic form, or stereoisomer thereof, wherein Ring C, Ring E, L, R^{2′}, R³, R⁴, R⁵, R^{5′}, W, n, p, s, and y are as defined above for Formula I and described in classes and subclasses herein, both singly and in combination. In some embodiments, the present disclosure provides a compound of formula IF1 or a pharmaceutically acceptable salt thereof.

In some embodiments in any formulae described herein, Ring A is phenyl or a 5- to 6-membered heteroaryl ring having 1-2 heteroatoms independently selected from nitrogen, oxygen, and sulfur. In some embodiments, Ring A is phenyl or a 6-membered heteroaryl ring having 1-2 heteroatoms independently selected from nitrogen, oxygen, and sulfur.

In some embodiments of any formulae described herein, Ring A is phenyl. In some embodiments, Ring A

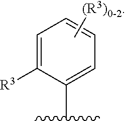

In some embodiments, Ring A is

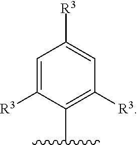

In some embodiments, Ring A is

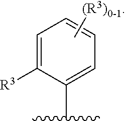

In some embodiments, Ring A is selected from

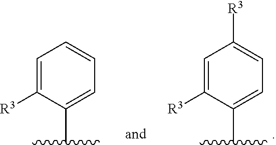

In some embodiments, Ring A is selected from

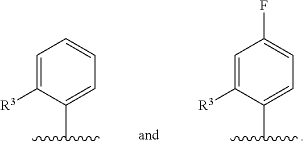

In some embodiments, Ring A is

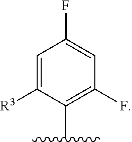

In some embodiments of any formulae described herein, Ring A is a 5- to 6-membered heteroaryl ring having 1-3 heteroatoms independently selected from nitrogen, oxygen, and sulfur. In some embodiments, Ring A is a 5- to 6-membered heteroaryl ring having 1-2 heteroatoms independently selected from nitrogen, oxygen, and sulfur. In some embodiments, Ring A is a 5-membered heteroaryl ring having 1-3 heteroatoms independently selected from nitrogen, oxygen, and sulfur. In some embodiments, Ring A is a pyrazole. In some embodiments, Ring A is a 6-membered heteroaryl ring having 1-3 nitrogen atoms. In some embodiments, Ring A is a pyridine or pyridone.

In some embodiments of any formulae described herein, Ring A, substituted with m instances of R³, is selected from

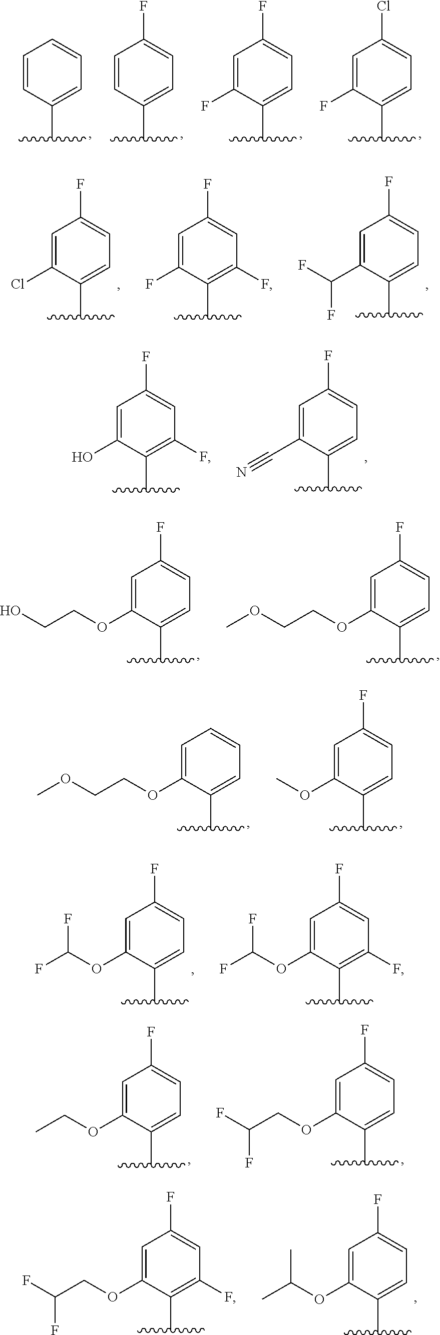

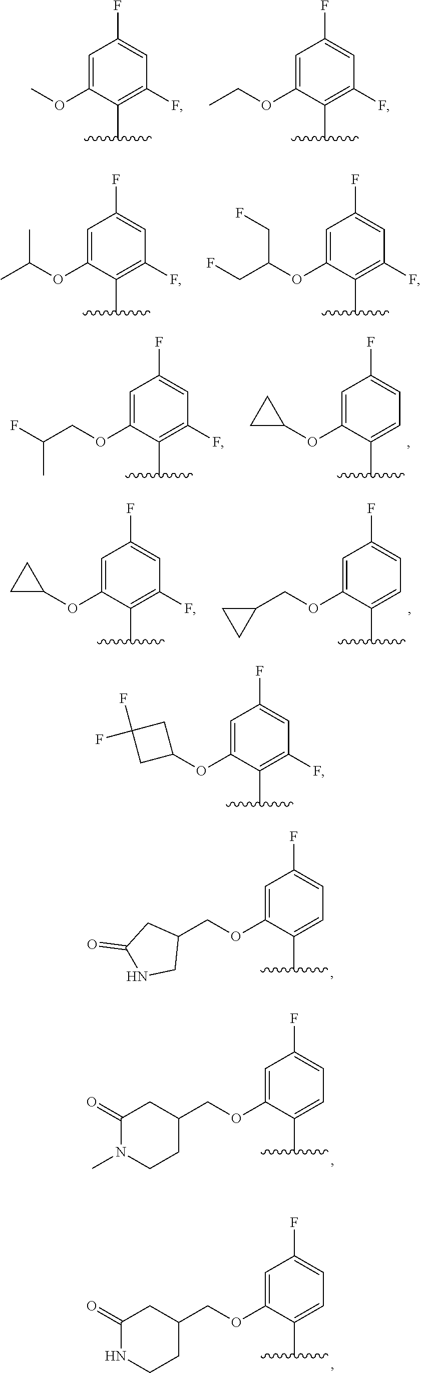

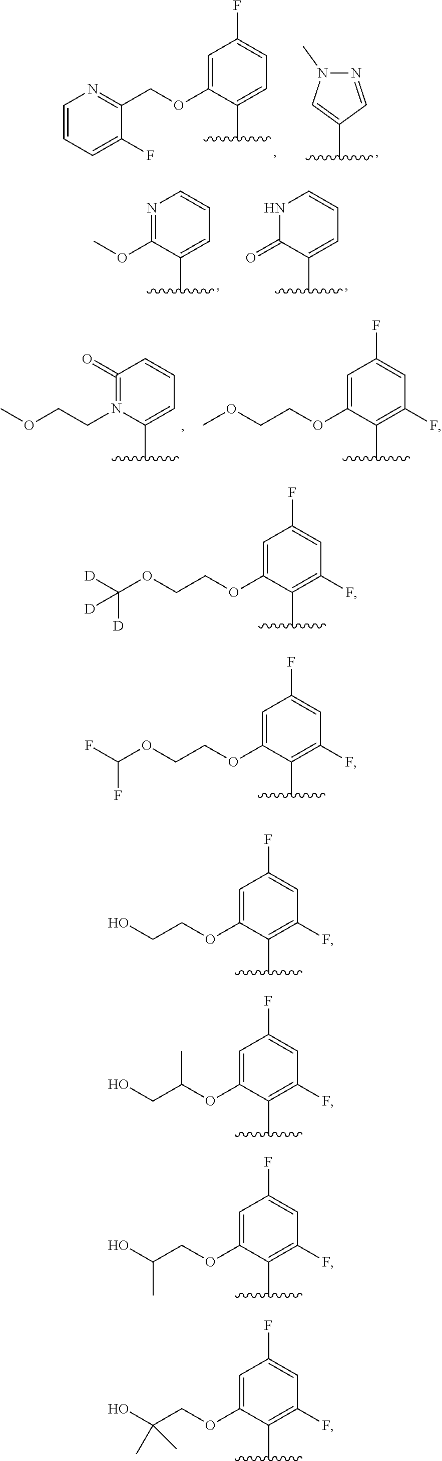

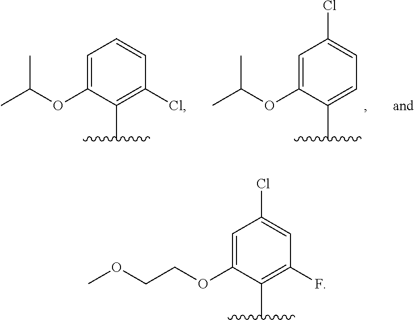

In some embodiments of any formulae described herein, each R³ is independently selected from oxo, halogen (e.g., F or Cl), —CN, —OR, —O(CH₂)ᵥCy, —O—(C₁₋₄ alkylene)-OR (e.g., —OCH₂CH₂OR), —CH₂CH₂OR, C₁₋₆ aliphatic, and C₁₋₆ haloaliphatic. In some embodiments, each R³ is independently selected from oxo, halogen (e.g., F or Cl), —CN, —OR, —O(CH₂)ᵥCy, —OCH₂CH₂OR, —CH₂CH₂OR, C₁₋₆ aliphatic, and C₁₋₆ haloaliphatic. In some embodiments, each R³ is independently selected from halogen, —CN, —OR, —O(CH₂)ᵥCy, —OCH₂CH₂OR, and optionally substituted C₁₋₆ aliphatic. In some embodiments, each R³ is independently selected from oxo, halogen, —CN, —OR, —O(CH₂)ᵥCy, —OCH₂CH₂OR, and optionally substituted C₁₋₆ alkyl. In some embodiments, each R³ is independently selected from oxo, halogen, —CN, —OR, —O(CH₂)ᵥCy, —OCH₂CH₂OR, and C₁₋₆ aliphatic optionally substituted with one or more halogen or —OR^{∘}. In some such embodiments, R^{∘} is C₁₋₄ alkyl. Accordingly, in some embodiments, each R³ is independently selected from oxo, halogen, —CN, —OR, —O(CH₂)ᵥCy, —OCH₂CH₂OR, and C₁₋₆ aliphatic optionally substituted with one or more halogen or —O(C₁₋₄ alkyl). In some embodiments, each R³ is independently selected from oxo, halogen, —CN, —OR, —O(CH₂)ᵥCy, —OCH₂CH₂OR, and C₁₋₆ alkyl optionally substituted with one or more halogen or —O(C₁₋₄ alkyl). In some embodiments, at least one R³ is selected from halogen, —OR, —O(CH₂)ᵥCy, and —OCH₂CH₂OR. In some embodiments, at least one R³ is selected from —OR, —O(CH₂)ᵥCy, and —OCH₂CH₂OR. In some embodiments, Ring A is

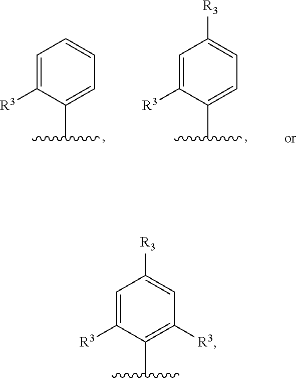

wherein each R³ is independently selected from halogen, —OR, —O(CH₂)ᵥCy, and —OCH₂CH₂OR. In some embodiments, Ring A is

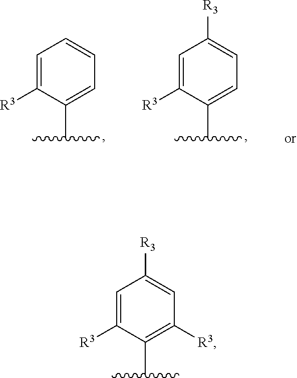

wherein each R³ is independently selected from halogen, —OR, —O(CH₂)ᵥCy, and —O—(C₁₋₄ alkylene)-OR. In some embodiments, Ring A is

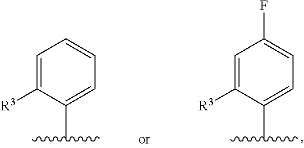

wherein R³ is selected from —OR, —O(CH₂)ᵥCy, and —OCH₂CH₂OR. In some embodiments, Ring A is

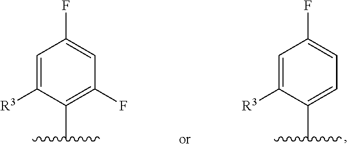

wherein R³ is selected from —OR, —O(CH₂)ᵥCy, and —O—(C₁₋₄ alkylene)-OR.

In some embodiments of any formulae described herein, each R³ is independently selected from halogen, —OR, —OCH₂CH₂OR, and C₁₋₆ alkyl optionally substituted with one or more halogen or —O(C₁₋₄ alkyl). In some embodiments, at least one R³ is selected from halogen (e.g., F). In some embodiments, at least one R³ is selected from —OCH₂CH₂OR, and C₁₋₆ alkyl optionally substituted with one or more halogen or —O(C₁₋₄ alkyl). In some embodiments, at least one is R³ selected from —OCH₃, —OCH₂CH₃, —OCH(CH₃)₂, —OCHF₂, —OCH₂CHF₂, —OCH(CH₂F)₂, —OCH₂CH₂OH, —OCH₂CH₂OCH₃, —OCH₂CH(CH₃)OH, —OCH₂C(CH₃)₂OH, —OCH(CH₃)CH₂OH, and —O(cyclopropyl).

In some embodiments of any formulae described herein, R³ is oxo (e.g., when Ring A is not phenyl). In some embodiments, R³ is halogen (e.g., fluoro or chloro). In some embodiments, R³ is fluoro. In some embodiments, R³ is chloro. In some embodiments, R³ is —CN. In some embodiments, R³ is —OR. In some embodiments, R³ is —O(C₁₋₄ alkyl optionally substituted with —OH) or —O(C₁₋₄ haloalkyl). In some embodiments, R³ is —OCH₃, —OCH₂CH₃, —OCH(CH₃)₂, —OCH(CH₃)CH₂OH, —OCH₂CH(CH₃)OH, —OCH₂C(CH₃)₂OH, —OCHF₂, —OCH₂CHF₂, or —OCH(CH₂F)₂. In some embodiments, R³ is —O(C₁₋₄ alkyl) or —O(C₁₋₄ haloalkyl). In some embodiments, R³ is —OCH₃, —OCH₂CH₃, —OCH(CH₃)₂, —OCHF₂, or —OCH₂CHF₂. In some embodiments, R³ is —O(CH₂)ᵥCy. In some embodiments, R³ is —OCH₂Cy. In some embodiments, R³ is —O(CH₂)ᵥCy, wherein Cy is a 5- to 6-membered heteroaryl ring having 1-3 heteroatoms independently selected from nitrogen, oxygen, and sulfur, substituted with 0-2 instances of R⁶ (e.g., Cy is a pyridine substituted with fluoro). In some embodiments, R³ is —O(CH₂)ᵥCy, wherein Cy is a 3- to 6-membered carbocyclic ring substituted with 0-2 instances of R⁶ (e.g., Cy is a cyclopropane or cyclobutane substituted with one or more halogens). In some embodiments, R³ is —O(CH₂)ᵥCy, wherein Cy is a 3- to 6-membered carbocyclic ring (e.g., Cy is a cyclopropane). In some embodiments. R³ is —O(CH₂)ᵥCy, wherein Cy is a 4- to 6-membered heterocyclic ring having 1-2 heteroatoms independently selected from nitrogen, oxygen, and sulfur (e.g., Cy is a pyrrolidone or piperidinone optionally substituted with C₁₋₆ alkyl). In some embodiments, R³ is —O—(C₁₋₄ alkylene)-OR. In some embodiments, R³ is —O—(C₁₋₄ alkylene)-OR. In some embodiments, R³ is —OCH₂CH₂OH, —OCH(CH₃)CH₂OH, —OCH₂CH(CH₃)OH, —OCH₂C(CH₃)₂OH, —OCH₂CH₂O(C₁₋₄ alkyl), or —OCH₂CH₂O(C₁₋₄ haloalkyl). In some embodiments, R³ is —OCH₂CH₂OR. In some embodiments, R³ is —OCH₂CH₂OH or —OCH₂CH₂O(C₁₋₄ alkyl). In some embodiments, R³ is —CH₂CH₂OR. In some embodiments, R³ is —CH₂CH₂O(C₁₋₄ alkyl). In some embodiments, R³ is optionally substituted C₁₋₆ aliphatic. In some embodiments, R³ is optionally substituted C₁₋₆ alkyl. In some embodiments, R³ is C₁₋₆ aliphatic optionally substituted with one or more halogen (e.g., fluoro) or —OR^{∘} (e.g., —O(C₁₋₄ alkyl)). In some embodiments, R³ is C₁₋₆ alkyl optionally substituted with one or more halogen (e.g., fluoro) or —OR^{∘} (e.g., —O(C₁₋₄ alkyl)). In some embodiments, R³ is C₁₋₆ haloaliphatic. In some embodiments, R³ is C₁₋₆ haloalkyl.

In some embodiments of any formulae described herein, each R³ is independently selected from oxo, fluoro, chloro, —CN, —OH, —OCH₃, —OCH₂CH₃, —OCH(CH₃)₂, —OCHF₂, —OCH₂CHF₂, —OCH(CH₂F)₂, —OCH₂CH₂OH, —OCH₂CH₂OCH₃, —OCH₂CH₂OCD₃, —OCH(CH₃)CH₂OH, —OCH₂CH(CH₃)OH, —OCH₂C(CH₃)₂OH, —OCH₂CH₂OCHF₂, —CH₃, —CHF₂, —CH₂CH₂OCH₃,

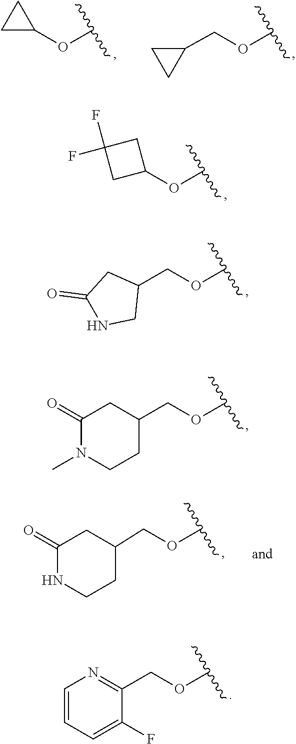

In some embodiments of any formulae described herein, m is 1, 2, or 3. In some embodiments, m is 1 or 2. In some embodiments, m is 0. In some embodiments, m is 1. In some embodiments, m is 2. In some embodiments, m is 3.

In some embodiments of any formulae described herein, Ring B is selected from a 6-membered heteroaryl ring having 1-3 nitrogen atoms and a 9- to 10-membered bicyclic ring having 1-3 heteroatoms independently selected from nitrogen, oxygen, and sulfur, wherein the bicyclic ring comprises at least one 5- or 6-membered heteroaryl ring having at least one nitrogen atom. In some embodiments, Ring B is selected from a 6-membered heteroaryl ring having 1-2 nitrogen atoms and a 9- to 10-membered bicyclic ring having 1-3 heteroatoms independently selected from nitrogen, oxygen, and sulfur, wherein the bicyclic ring comprises at least one 5- or 6-membered heteroaryl ring having at least one nitrogen atom. In some embodiments, Ring B is selected from a 6-membered heteroaryl ring having 1-2 nitrogen atoms and a 9-membered bicyclic ring having 1-3 heteroatoms independently selected from nitrogen, oxygen, and sulfur, wherein the bicyclic ring comprises at least one 5- or 6-membered heteroaryl ring having at least one nitrogen atom.

In some embodiments of any formulae described herein, Ring B is phenyl.

In some embodiments of any formulae described herein, Ring B is a 6-membered heteroaryl ring having 1-2 nitrogen atoms. In some embodiments, Ring B is a pyridine. In some embodiments, Ring B is a pyrazine. In some embodiments, Ring B is a pyrimidine. In some embodiments, Ring B is a pyridazine.

In some embodiments of any formulae described herein, Ring B is a 9- to 10-membered bicyclic ring that comprises at least one 5- or 6-membered heteroaryl ring. In some embodiments, Ring B is a 9- to 10-membered bicyclic ring that comprises at least one 5- or 6-membered heteroaryl ring comprising at least one nitrogen atom. In some embodiments, Ring B is a 9- to 10-membered bicyclic ring that comprises one 5- or 6-membered heteroaryl ring comprising at least one nitrogen atom. In some embodiments, Ring B is a 9- to 10-membered bicyclic ring having 1-3 heteroatoms independently selected from nitrogen, oxygen, and sulfur, wherein the bicyclic ring comprises at least one 5- or 6-membered heteroaryl ring having at least one nitrogen atom. In some embodiments, Ring B is a 9- to 10-membered bicyclic ring that comprises at least one 6-membered heteroaryl ring. In some embodiments, Ring B is a 9- to 10-membered bicyclic ring that comprises at least one 6-membered heteroaryl ring comprising at least one nitrogen atom. In some embodiments, Ring B is a 9- to 10-membered bicyclic ring having 1-3 heteroatoms independently selected from nitrogen, oxygen, and sulfur, wherein the bicyclic ring comprises at least one 6-membered heteroaryl ring having at least one nitrogen atom. In some embodiments, Ring B is a 9-membered bicyclic ring that comprises at least one 5- or 6-membered heteroaryl ring. In some embodiments, Ring B is a 9-membered bicyclic ring that comprises at least one 5- or 6-membered heteroaryl ring comprising at least one nitrogen atom. In some embodiments, Ring B is a 9-membered bicyclic heteroaryl ring that comprises at least one 5- or 6-membered heteroaryl ring comprising at least one nitrogen atom. In some embodiments, Ring B is a 9- to 10-membered bicyclic ring that comprises a pyridine ring. In some embodiments, Ring B is a 9- to 10-membered bicyclic ring that comprises a pyrazine ring. In some embodiments, Ring B is a 9- to 10-membered bicyclic ring that comprises a pyrazole ring. In some embodiments, Ring B is a 9- to 10-membered bicyclic ring that comprises a thiazole ring. In some embodiments, Ring B is a 9- to 10-membered bicyclic ring that comprises a thiophene ring. In some embodiments, Ring B is a 9- to 10-membered bicyclic ring that comprises a furan ring. In some embodiments, Ring B is a 9- to 10-membered bicyclic ring that comprises a cyclopentane or cyclopentene ring. In some embodiments, Ring B is a thieno[3,2-c]pyridine, thieno[2,3-c]pyridine, thiazolo[4,5-c]pyridine, pyrazolo[1,5-a]pyrazine, furo[3,2-c]pyridine, or 6,7-dihydro-5H-cyclopenta[c]pyridine. In some embodiments, Ring B is a 10-membered bicyclic ring that comprises at least one 6-membered heteroaryl ring. In some embodiments, Ring B is a 10-membered bicyclic ring that comprises at least one 6-membered heteroaryl ring comprising at least one nitrogen atom.

In some embodiments of any formulae described herein, Ring B, substituted with n instances of R⁴, is selected from

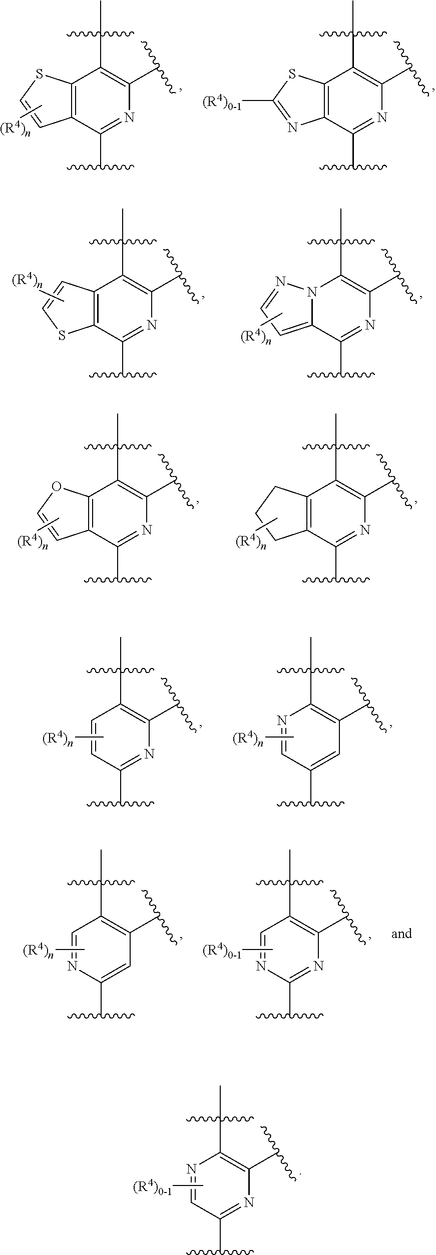

In some embodiments of any formulae described herein, Ring B, substituted with n instances of R⁴, is selected from

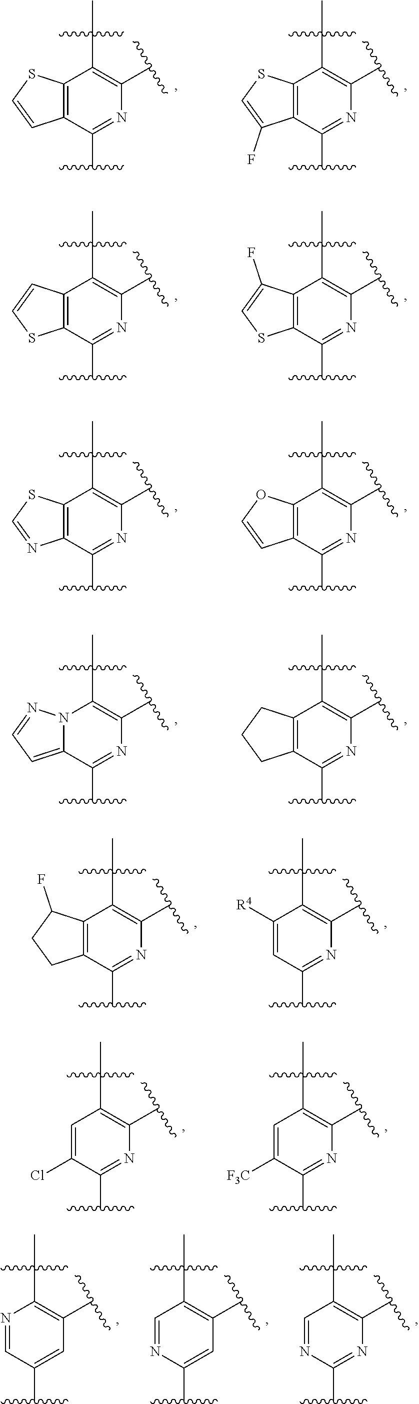

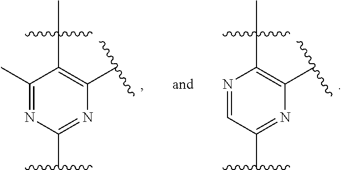

In some embodiments, Ring B, substituted with n instances of R⁴, is

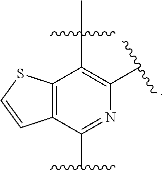 [SMILES: *c1nc(*)c2ccsc2c1*]

In some embodiments of any formulae described herein, each R⁴ is independently selected from halogen, C₁₋₆ aliphatic, and C₁₋₆ haloaliphatic. In some embodiments, each R⁴ is independently selected from halogen and C₁₋₆ aliphatic. In some embodiments, each R⁴ is independently selected from halogen and optionally substituted C₁₋₆ alkyl. In some embodiments, R⁴ is halogen (e.g., fluoro or chloro). In some embodiments, R⁴ is fluoro. In some embodiments, R⁴ is chloro. In some embodiments, R⁴ is optionally substituted C₁₋₆ aliphatic. In some embodiments, R⁴ is C₁₋₆ aliphatic. In some embodiments, R⁴ is C₁₋₆ haloaliphatic. In some embodiments, R⁴ is optionally substituted C₁₋₆ alkyl. In some embodiments, R⁴ is C₁₋₆ haloalkyl. In some embodiments, R⁴ is —CF₃. In some embodiments, R⁴ is C₁₋₆ alkyl. In some embodiments, R⁴ is methyl.

In some embodiments of any formulae described herein, n is 0 or 1. In some embodiments, n is 0. In some embodiments, n is 1. In some embodiments, n is 2.

In some embodiments of any formulae described herein, Ring C is selected from phenyl; a 5- to 6-membered heteroaryl ring having 1-3 heteroatoms independently selected from nitrogen, oxygen, and sulfur; a 5- to 8-membered bicyclic carbocyclic ring; a 4- to 8-membered saturated or partially unsaturated heterocyclic ring having 1-2 heteroatoms independently selected from nitrogen, oxygen, and sulfur; and a 7- to 10-membered saturated or partially unsaturated spirofused heterocyclic ring having 1-3 heteroatoms independently selected from nitrogen, oxygen, and sulfur, wherein each of the phenyl, heteroaryl, and heterocyclic rings is optionally fused to Ring E. In some embodiments, Ring C is selected from phenyl; a 5- to 6-membered heteroaryl ring having 1-3 heteroatoms independently selected from nitrogen, oxygen, and sulfur; a 5- to 6-membered saturated or partially unsaturated heterocyclic ring having 1-2 heteroatoms independently selected from nitrogen, oxygen, and sulfur; and a 9- to 10-membered saturated or partially unsaturated spirofused heterocyclic ring having 1-3 heteroatoms independently selected from nitrogen, oxygen, and sulfur, wherein each of the phenyl, heteroaryl, and heterocyclic rings is optionally fused to Ring E. In some embodiments, Ring C is phenyl; a 5- to 6-membered heteroaryl ring having 1-3 heteroatoms independently selected from nitrogen, oxygen, and sulfur; a 5- to 8-membered bicyclic carbocyclic ring; a 4- to 8-membered heterocyclic ring having 1-2 heteroatoms independently selected from nitrogen, oxygen, and sulfur; and a 7- to 10-membered spirofused heterocyclic ring having 1-3 heteroatoms independently selected from nitrogen, oxygen, and sulfur. In some embodiments, Ring C is phenyl; a 5- to 6-membered heteroaryl ring having 1-3 heteroatoms independently selected from nitrogen, oxygen, and sulfur; a 5- to 6-membered heterocyclic ring having 1-2 heteroatoms independently selected from nitrogen, oxygen, and sulfur; and a 9- to 10-membered spirofused heterocyclic ring having 1-3 heteroatoms independently selected from nitrogen, oxygen, and sulfur. In some embodiments, Ring C is phenyl; a 5- to 6-membered heteroaryl ring having 1-3 heteroatoms independently selected from nitrogen, oxygen, and sulfur; a 4- to 8-membered heterocyclic ring having 1-2 heteroatoms independently selected from nitrogen, oxygen, and sulfur; and a 7- to 10-membered spirofused heterocyclic ring having 1-3 heteroatoms independently selected from nitrogen, oxygen, and sulfur, wherein Ring C is fused to Ring E. In some embodiments, Ring C is phenyl; a 5- to 6-membered heteroaryl ring having 1-3 heteroatoms independently selected from nitrogen, oxygen, and sulfur; a 5- to 6-membered heterocyclic ring having 1-2 heteroatoms independently selected from nitrogen, oxygen, and sulfur; and a 9- to 10-membered spirofused heterocyclic ring having 1-3 heteroatoms independently selected from nitrogen, oxygen, and sulfur, wherein Ring C is fused to Ring E.

In some embodiments of any formulae described herein, Ring C is not fused to Ring E. In some embodiments, Ring C is fused to Ring E. In some embodiments, when Ring C is fused to Ring E, only one of Ring C and Ring E is aromatic.

It will be appreciated that, throughout the present disclosure, when a ring is described as being fused to another ring, one of skill in the art will understand that such fusion may or may not result in additional unsaturation in one or both of the rings that is not accounted for in the name of each ring alone.

For example, a cyclopentane ring fused to a phenyl ring has the following structure:

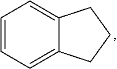

and a phenyl ring fused to a pyrrole ring has the following structure:

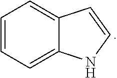

In some embodiments of any formulae described herein, Ring C is phenyl. In some embodiments, Ring C is phenyl that is not fused to Ring E. In some embodiments, Ring C is phenyl fused to Ring E.

In some embodiments of any formulae described herein, Ring C is a 5- to 6-membered heteroaryl ring having 1-3 heteroatoms independently selected from nitrogen, oxygen, and sulfur. In some embodiments, Ring C is a 5- to 6-membered heteroaryl ring having 1-3 heteroatoms independently selected from nitrogen, oxygen, and sulfur, wherein Ring C is not fused to Ring E. In some embodiments, Ring C is a 5- to 6-membered heteroaryl ring having 1-3 heteroatoms independently selected from nitrogen, oxygen, and sulfur, wherein Ring C is fused to Ring E. In some embodiments, Ring C is a 5-membered heteroaryl ring having 1-3 heteroatoms independently selected from nitrogen, oxygen, and sulfur. In some embodiments, Ring C is a pyrazole, imidazole, or triazole. In some embodiments, Ring C is a pyrazole, imidazole, triazole, oxazole, thiazole, or oxadiazole. In some embodiments, Ring C is 6-membered heteroaryl ring having 1-3 nitrogen atoms. In some embodiments, Ring C is a pyridine, pyridone, pyrimidine, or pyridazinone. In some embodiments, Ring C is a pyridine, pyridone, pyrazine, pyrimidine, pyridazine, or pyridazinone.

In some embodiments of any formulae described herein, Ring C is a 5- to 8-membered bicyclic carbocyclic ring. In some embodiments, Ring C is a 5- to 8-membered bridged bicyclic carbocyclic ring. In some embodiments, Ring C is a bridged cyclobutane (e.g., bicyclo[1.1.1]pentane).

In some embodiments of any formulae described herein, Ring C is a 4- to 8-membered heterocyclic ring having 1-2 heteroatoms independently selected from nitrogen, oxygen, and sulfur. In some embodiments, Ring C is a 5- to 6-membered heterocyclic ring having 1-2 heteroatoms independently selected from nitrogen, oxygen, and sulfur. In some embodiments, Ring C is a 4- to 8-membered saturated or partially unsaturated heterocyclic ring having 1-2 heteroatoms independently selected from nitrogen, oxygen, and sulfur, wherein Ring C is not fused to Ring E. In some embodiments, Ring C is a 4- to 8-membered saturated or partially unsaturated heterocyclic ring having 1-2 heteroatoms independently selected from nitrogen, oxygen, and sulfur, wherein Ring C is fused to Ring E. In some embodiments, Ring C is a 4- to 8-membered saturated or partially unsaturated heterocyclic ring having 1-2 heteroatoms independently selected from nitrogen, oxygen, and sulfur. In some embodiments, Ring C is a 5- to 6-membered saturated or partially unsaturated heterocyclic ring having 1-2 heteroatoms independently selected from nitrogen, oxygen, and sulfur. In some embodiments, Ring C is a 4-membered heterocyclic ring having 1 heteroatom independently selected from nitrogen, oxygen, and sulfur. In some embodiments, Ring C is an azetidine. In some embodiments, Ring C is a 5-membered heterocyclic ring having 1-2 heteroatoms independently selected from nitrogen, oxygen, and sulfur. In some embodiments, Ring C is a pyrrolidine. In some embodiments, Ring C is a 6-membered heterocyclic ring having 1-2 heteroatoms independently selected from nitrogen, oxygen, and sulfur. In some embodiments, Ring C is a piperidine, tetrahydropyridine, piperazine, piperazinone, or thiomorpholine-1,1-dioxide. In some embodiments, Ring C is a 7- to 8-membered heterocyclic ring having 1-2 heteroatoms independently selected from nitrogen, oxygen, and sulfur. In some embodiments, Ring C is a bridged ring system. In some embodiments, Ring C is a 7- to 8-membered bridged heterocyclic ring having 1-2 heteroatoms independently selected from nitrogen, oxygen, and sulfur. In some embodiments, Ring C is a bridged piperazine (e.g., 3,8-diazabicyclo[3.2.1]octane). In some embodiments, Ring C is a bridged piperidine (e.g., 3-azabicyclo[3.1.1]heptane).

In some embodiments of any formulae described herein, Ring C is a 7- to 10-membered spirofused heterocyclic ring having 1-3 heteroatoms independently selected from nitrogen, oxygen, and sulfur. In some embodiments, Ring C is a 9- to 10-membered spirofused heterocyclic ring having 1-3 heteroatoms independently selected from nitrogen, oxygen, and sulfur. In some embodiments, Ring C is a 7- to 10-membered saturated or partially unsaturated spirofused heterocyclic ring having 1-3 heteroatoms independently selected from nitrogen, oxygen, and sulfur, wherein Ring C is not fused to Ring E. In some embodiments, Ring C is a 7- to 10-membered saturated or partially unsaturated spirofused heterocyclic ring having 1-3 heteroatoms independently selected from nitrogen, oxygen, and sulfur, wherein Ring C is fused to Ring E. In some embodiments, Ring C is a 7- to 10-membered saturated or partially unsaturated spirofused heterocyclic ring having 1-3 heteroatoms independently selected from nitrogen, oxygen, and sulfur. In some embodiments, Ring C is a 9- to 10-membered saturated or partially unsaturated spirofused heterocyclic ring having 1-3 heteroatoms independently selected from nitrogen, oxygen, and sulfur. In some embodiments, Ring C is a 7-membered spirofused heterocyclic ring having two 4-membered rings. In some embodiments, Ring C is a 2-azaspiro[3.3]heptane or 2,6-diazaspiro[3.3]heptane. In some embodiments, Ring C is an 8-membered spirofused heterocyclic ring having a 4-membered ring and a 5-membered ring. In some embodiments, Ring C is a 2,6-diazaspiro[3.4]octane. In some embodiments, Ring C is a 9-membered spirofused heterocyclic ring having 1-3 heteroatoms independently selected from nitrogen, oxygen, and sulfur. In some embodiments, Ring C is a 9-membered spirofused heterocyclic ring having a 4-membered ring and a 6-membered ring. In some embodiments, Ring Cis 2-azaspiro[3.5]nonane, 2-azaspiro[3.5]non-6-ene, 2,7-diazaspiro[3.5]nonane, 2-oxa-7-azaspiro[3.5]nonane, or 2-thia-7-azaspiro[3.5]nonane-2,2-dioxide. In some embodiments, Ring C is a 10-membered spirofused heterocyclic ring having 1-3 heteroatoms independently selected from nitrogen, oxygen, and sulfur. In some embodiments, Ring C is a 2,8-diazaspiro[4.5]decan-3-one or 2,8-diazaspiro[4.5]decane.

In some embodiments of any formulae described herein, Ring C is not fused to Ring E, and Ring C, substituted with p instances of R⁵, is selected from

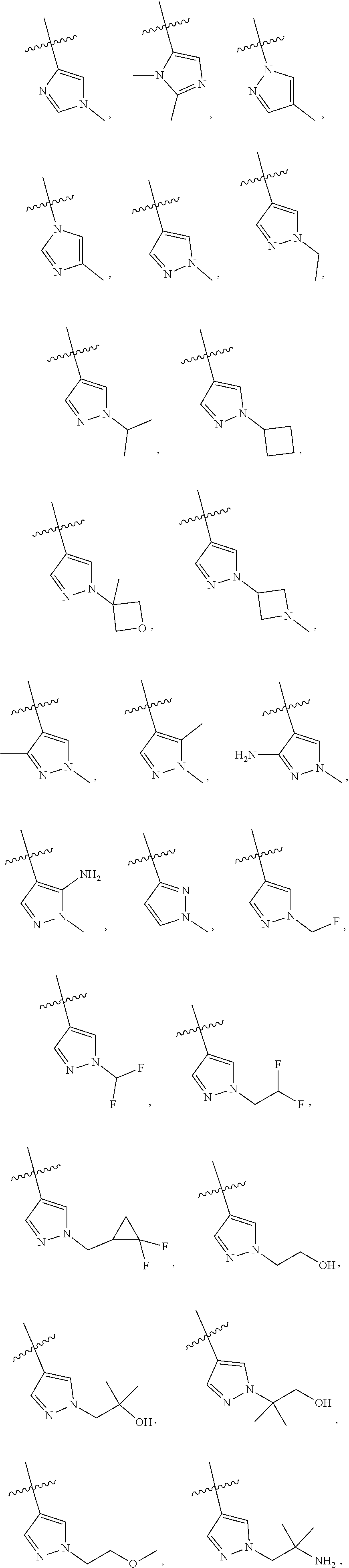

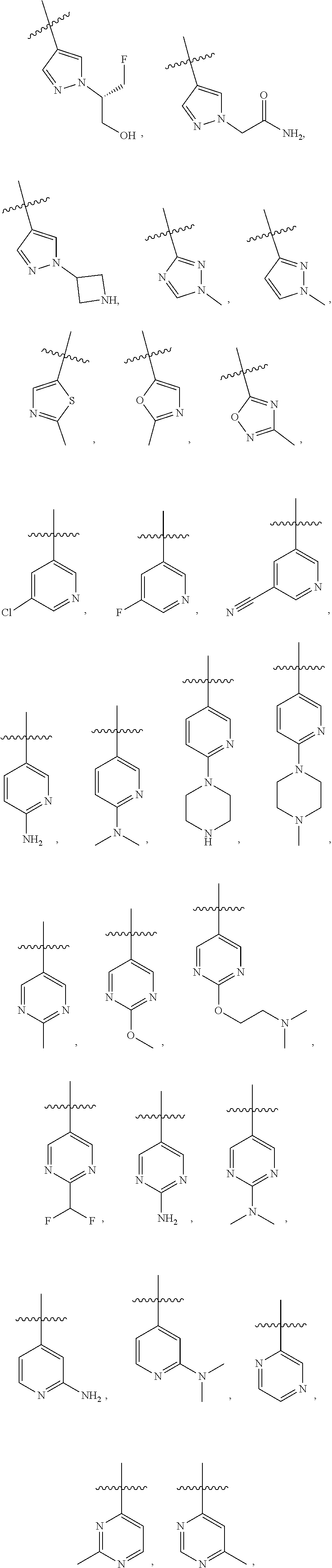

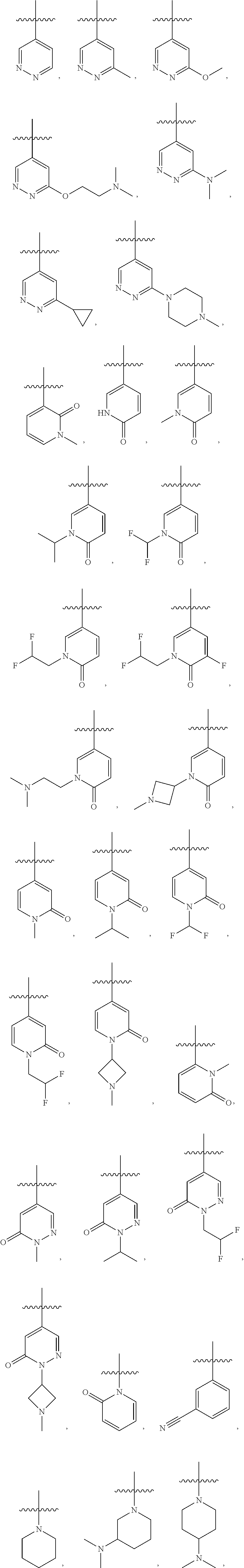

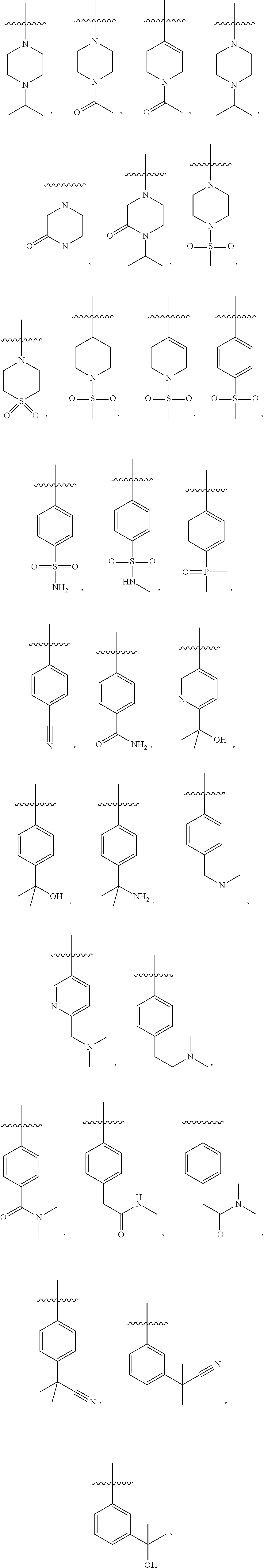

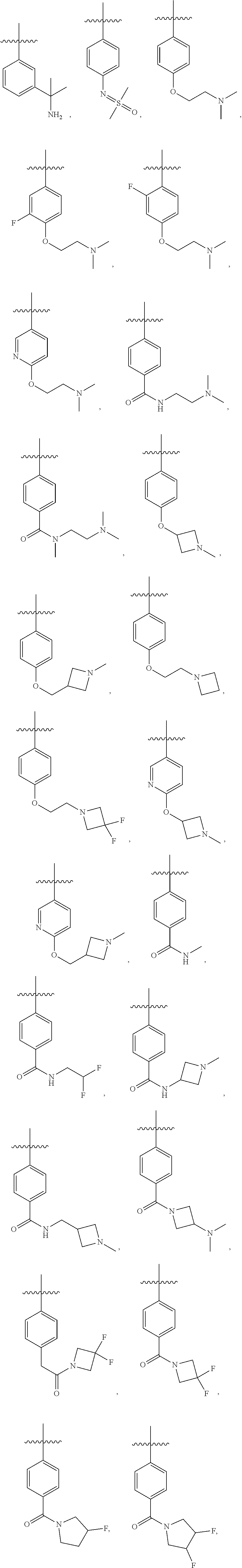

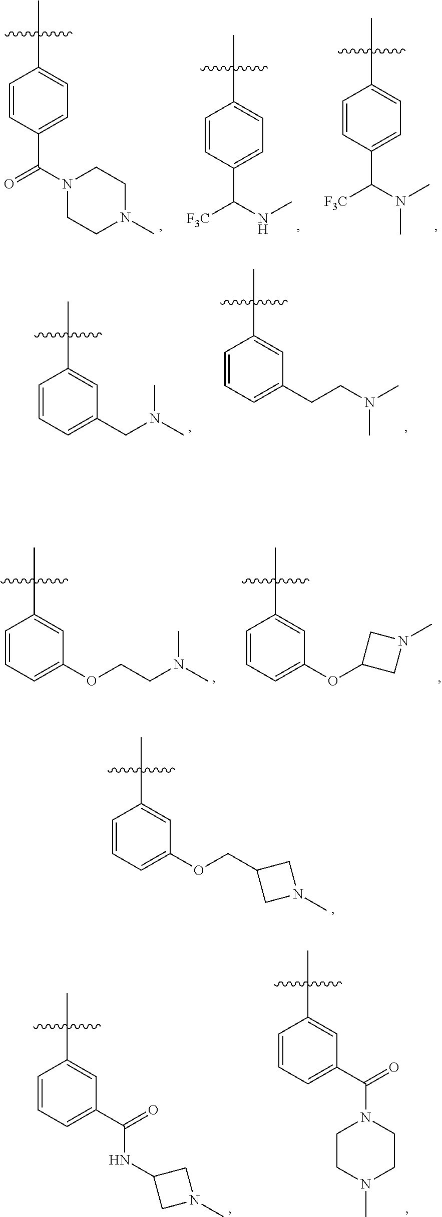

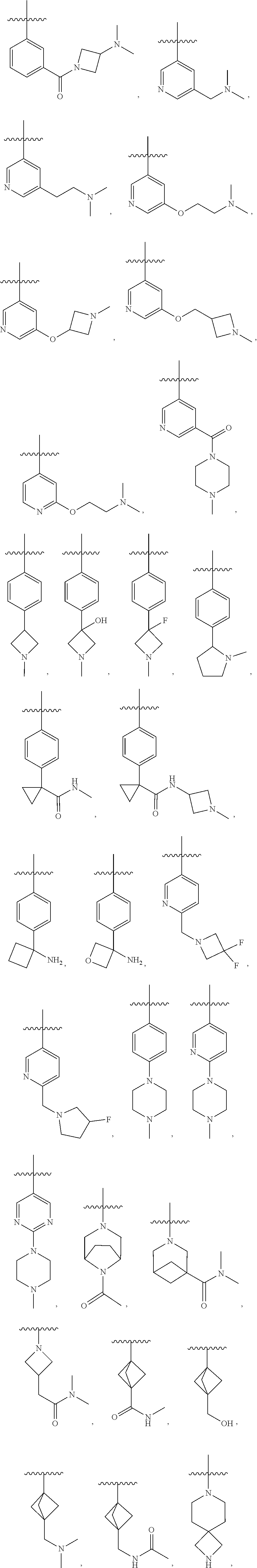

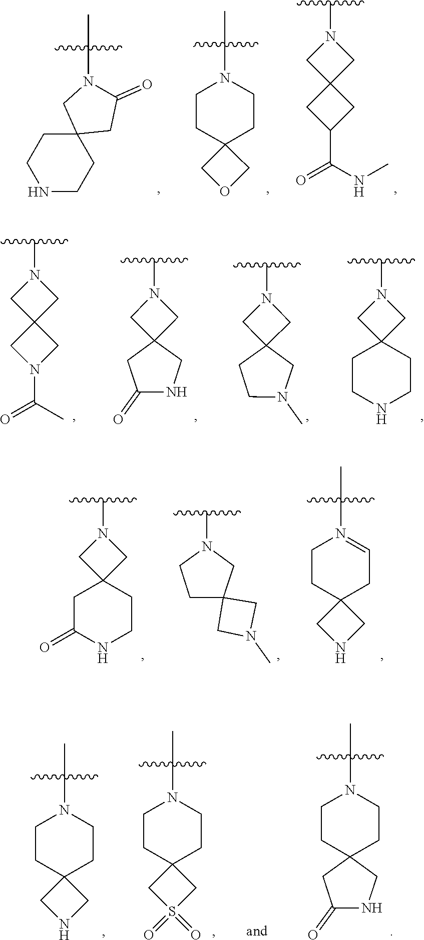

In some embodiments of any formulae described herein, Ring E is selected from a 5- to 6-membered saturated or partially unsaturated carbocyclic ring, a 5- to 7-membered saturated or partially unsaturated heterocyclic ring having 1-2 heteroatoms independently selected from nitrogen, oxygen, and sulfur, 7- to 10-membered saturated or partially unsaturated spirofused heterocyclic ring having 1-3 heteroatoms independently selected from nitrogen, oxygen, and sulfur, and a 5- to 6-membered heteroaryl ring having 1-3 heteroatoms independently selected from nitrogen, oxygen, and sulfur, wherein Ring E is substituted by s instances of R^{5′}. In some embodiments, Ring E is selected from a 5- to 6-membered saturated or partially unsaturated carbocyclic ring, a 5- to 7-membered saturated or partially unsaturated heterocyclic ring having 1-2 heteroatoms independently selected from nitrogen, oxygen, and sulfur, and a 5- to 6-membered heteroaryl ring having 1-3 heteroatoms independently selected from nitrogen, oxygen, and sulfur, wherein Ring E is substituted by s instances of R^{5′}.

In some embodiments of any formulae described herein, Ring E is a 5- to 6-membered carbocyclic ring substituted by s instances of R^{5′}. In some embodiments, Ring E is a 5- to 6-membered saturated or partially unsaturated carbocyclic ring substituted by s instances of R^{5′}. In some embodiments, Ring E is a 5-membered carbocyclic ring substituted by s instances of R^{5′}. In some embodiments, Ring E is a cyclopentane substituted by s instances of R^{5′}. In some embodiments, Ring E is a 6-membered carbocyclic ring substituted by s instances of R^{5′}. In some embodiments, Ring E is a cyclohexane substituted by s instances of R^{5′}.

In some embodiments of any formulae described herein, Ring E is a 5- to 7-membered heterocyclic ring having 1-2 heteroatoms independently selected from nitrogen, oxygen, and sulfur, wherein Ring E is substituted by s instances of R^{5′}. In some embodiments, Ring E is a 5- to 7-membered saturated or partially unsaturated heterocyclic ring having 1-2 heteroatoms independently selected from nitrogen, oxygen, and sulfur, wherein Ring E is substituted by s instances of R^{5′}. In some embodiments, Ring E is a 5-membered heterocyclic ring having 1-2 heteroatoms independently selected from nitrogen, oxygen, and sulfur, wherein Ring E is substituted by s instances of R^{5′}. In some embodiments, Ring E is a pyrrolidine, pyrrolidinone, 1-iminotetrahydro-1H-1λ⁶-thiophene-1-oxide, or tetrahydrothiophene-1,1-dioxide, each substituted by s instances of R^{5′}. In some embodiments, Ring E is a pyrrolidine, pyrrolidinone, imidazolidin-2-one, 1-iminotetrahydro-1H-1λ⁶-thiophene-1-oxide, or tetrahydrothiophene-1,1-dioxide, each substituted by s instances of R^{5′}. In some embodiments, Ring E is a 6-membered heterocyclic ring having 1-2 heteroatoms independently selected from nitrogen, oxygen, and sulfur, wherein Ring E is substituted by s instances of R^{5′}. In some embodiments, Ring E is a piperidine, piperidinone, hexahydropyrimidine, piperazine, or piperazinone, each substituted by s instances of R^{5′}. In some embodiments, Ring E is a 7-membered heterocyclic ring having 1-2 heteroatoms independently selected from nitrogen, oxygen, and sulfur, wherein Ring E is substituted by s instances of R^{5′}. In some embodiments, Ring E is an azepane substituted by s instances of R^{5′}. In some embodiments, Ring E is an azepane or 1,4-diazepan-2-one, each substituted by s instances of R^{5′}.

In some embodiments of any formulae described herein, Ring E is a 7- to 10-membered spirofused heterocyclic ring having 1-3 heteroatoms independently selected from nitrogen, oxygen, and sulfur, wherein Ring E is substituted by s instances of R^{5′}. In some embodiments, Ring E is a 8-membered spirofused heterocyclic ring having 1-2 heteroatoms independently selected from nitrogen, oxygen, and sulfur, wherein Ring E is substituted by s instances of R^{5′}. In some embodiments, Ring E is a 5-azaspiro[2.5]octane or a 5-azaspiro[2.5]octan-4-one, each substituted by s instances of R^{5′}.

In some embodiments of any formulae described herein, Ring E is a 5- to 6-membered heteroaryl ring having 1-3 heteroatoms independently selected from nitrogen, oxygen, and sulfur, wherein Ring E is substituted by s instances of R^{5′}. In some embodiments, Ring E is a 5-membered heteroaryl ring having 1-3 heteroatoms independently selected from nitrogen, oxygen, and sulfur, wherein Ring E is substituted by s instances of R^{5′}. In some embodiments, Ring E is a pyrazole or triazole, each substituted by s instances of R^{5′}. In some embodiments, Ring E is a pyrazole, imidazole, oxazole, or triazole, each substituted by s instances of R^{5′}. In some embodiments, Ring E is a 6-membered heteroaryl ring having 1-3 nitrogen atoms substituted by s instances of R^{5′}. In some embodiments, Ring E is a pyridazinone substituted by s instances of R^{5′}. In some embodiments, Ring E is a pyridone, pyrazine, pyrimidine, pyridazine, pyrazinone, pyrimidinone, or pyridazinone substituted by s instances of R^{5′}.

In some embodiments of any formulae described herein, Ring C is fused to Ring E, and

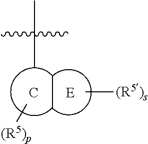

is selected from

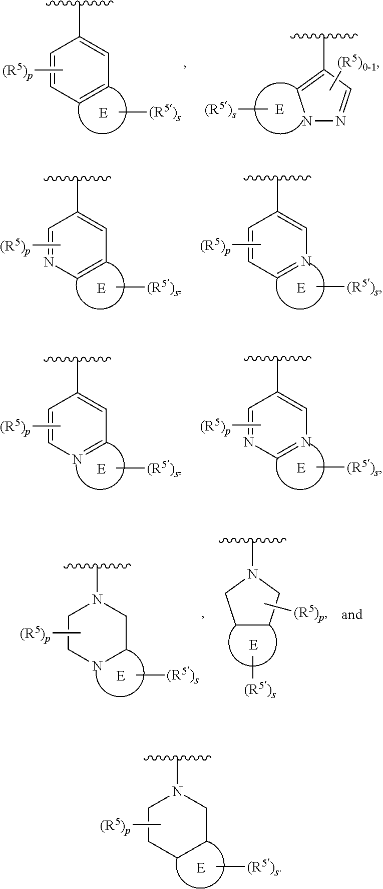

In some embodiments of any formulae described herein, Ring C is fused to Ring E, and Ring E, substituted with s instances of R^{5′}, is selected from

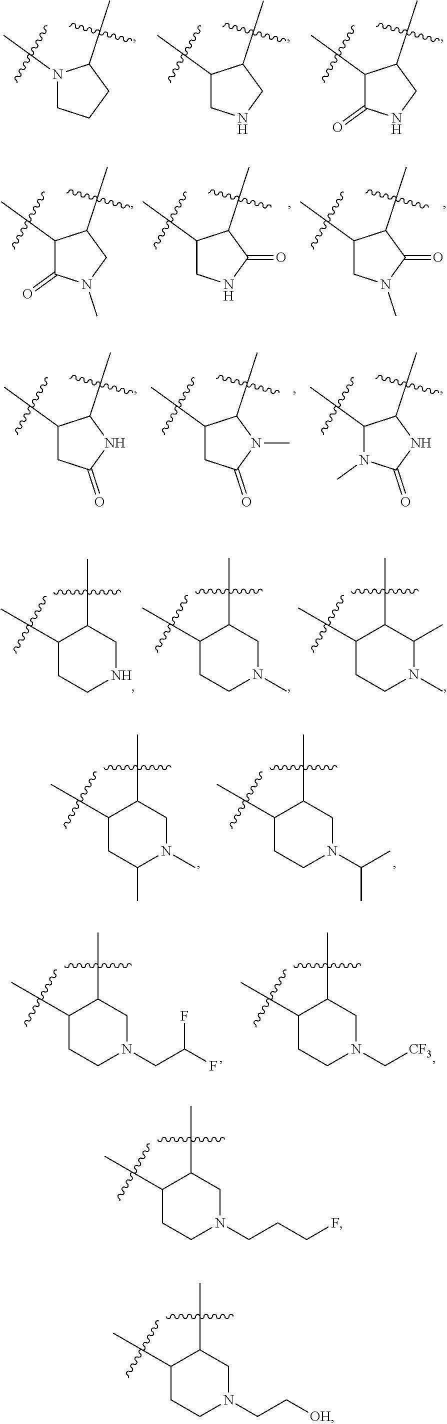

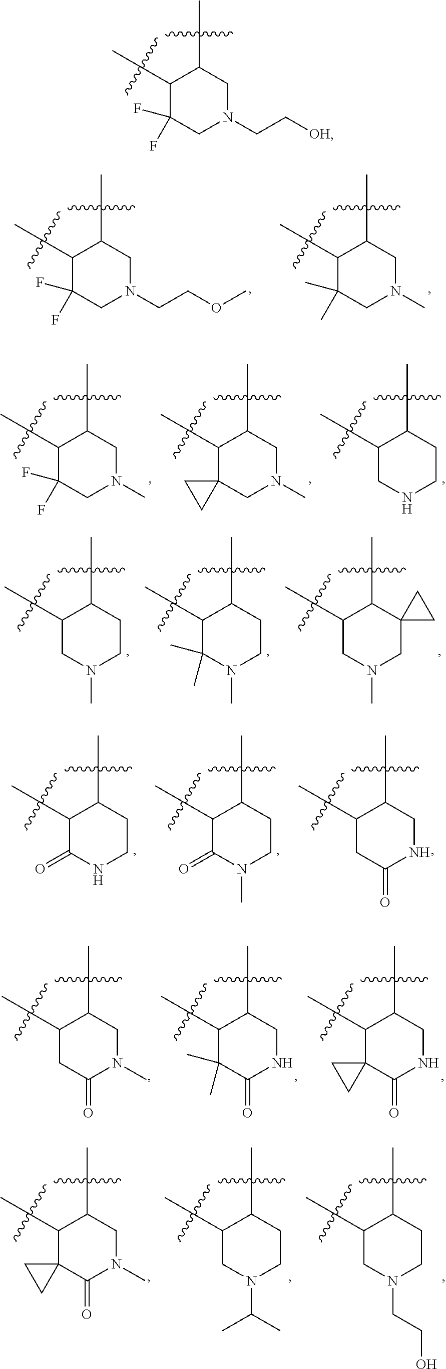

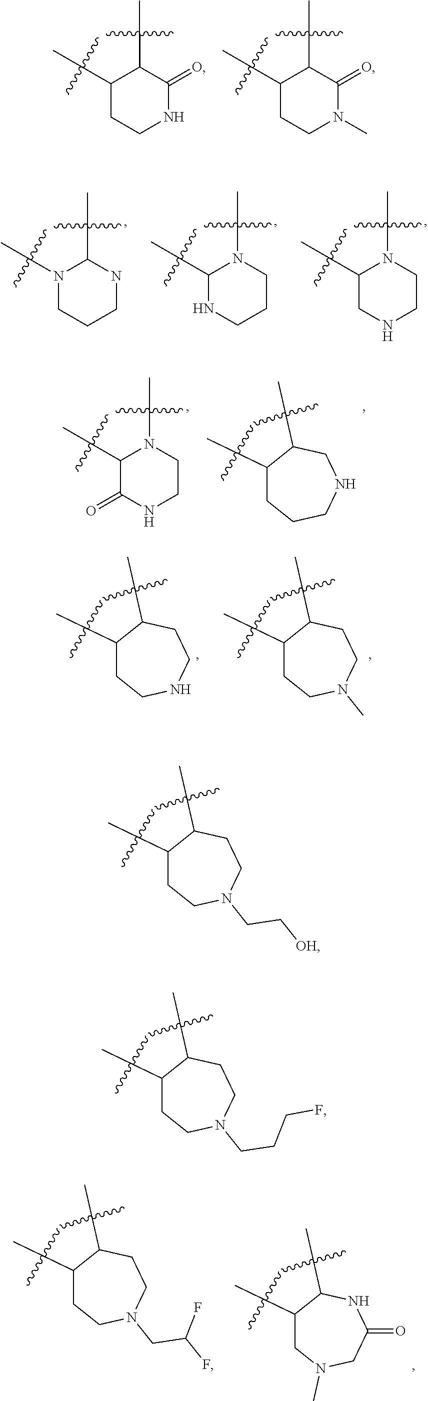

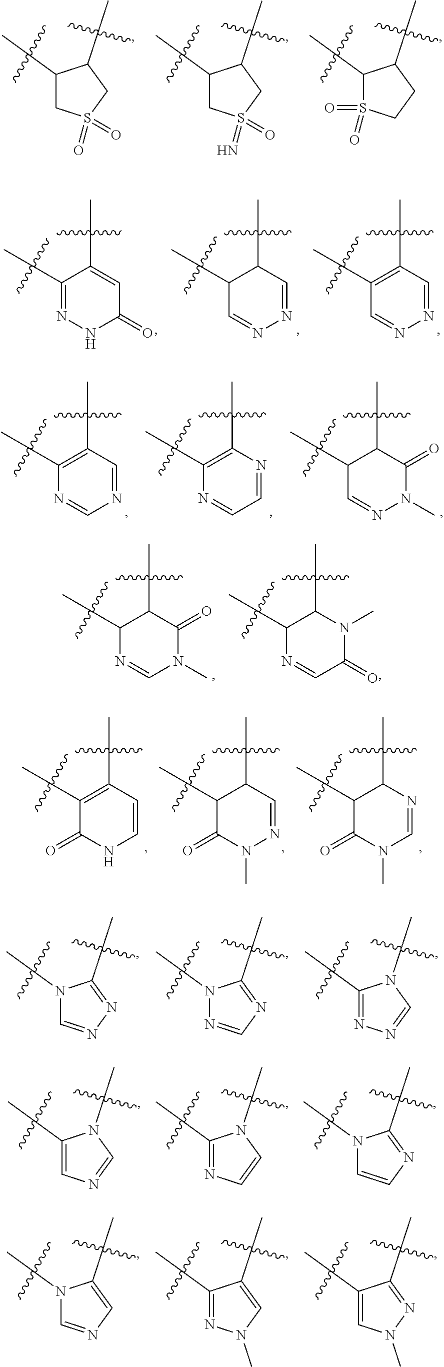

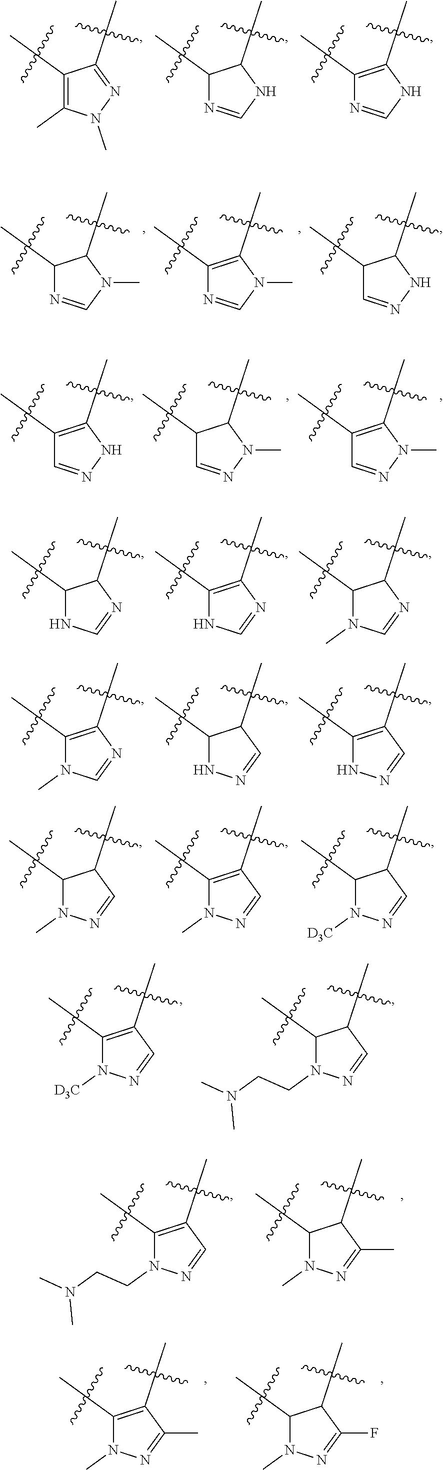

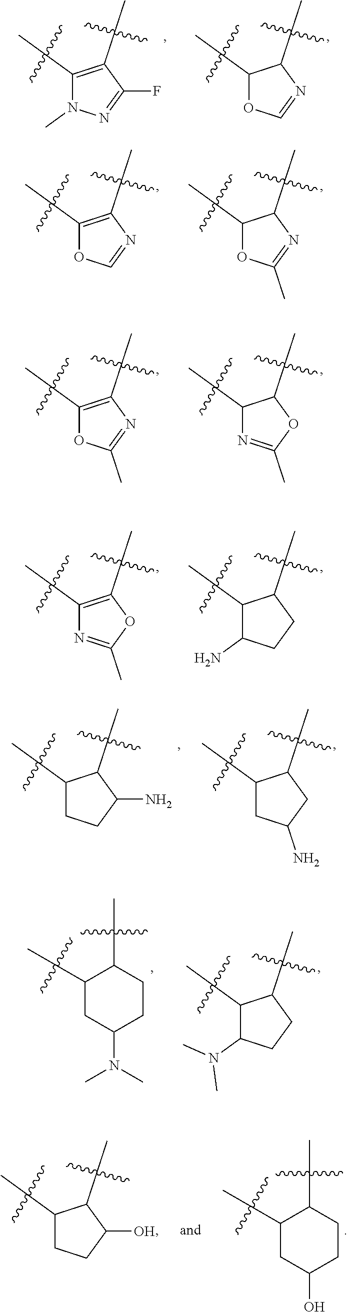

In some embodiments of any formulae described herein, Ring C is fused to Ring E, and

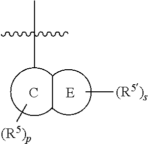

is selected from

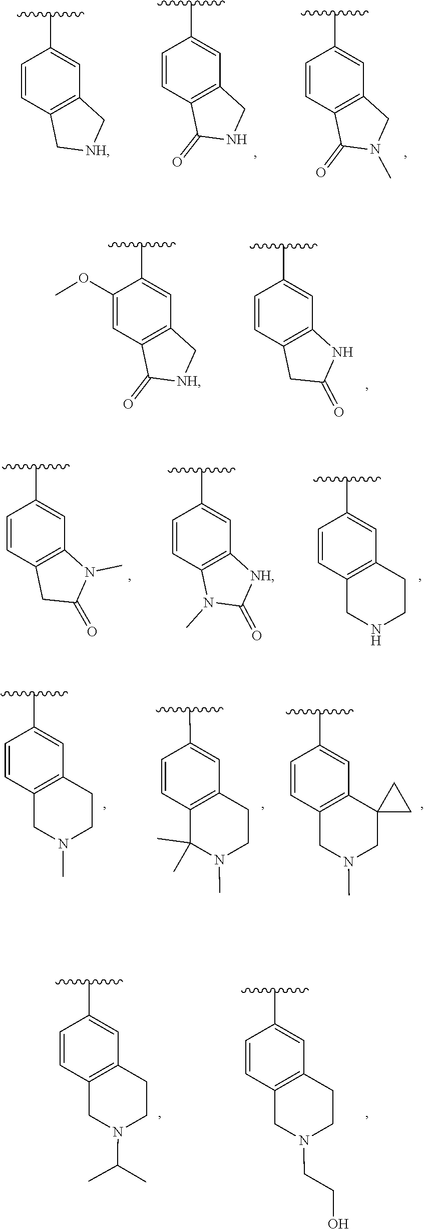

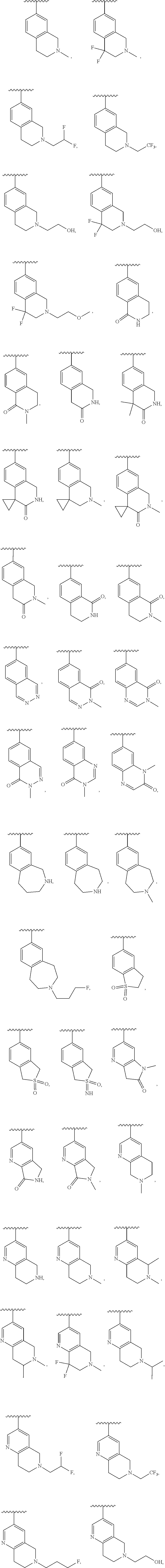

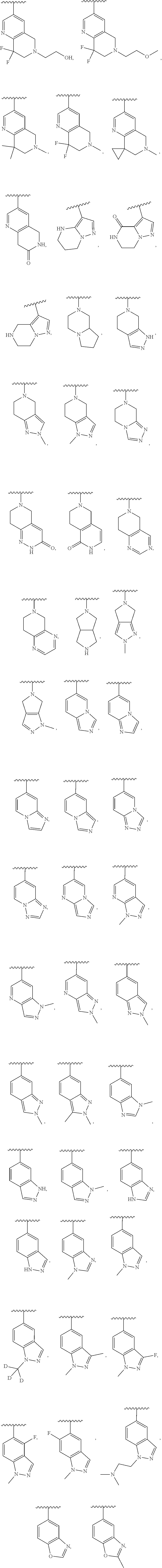

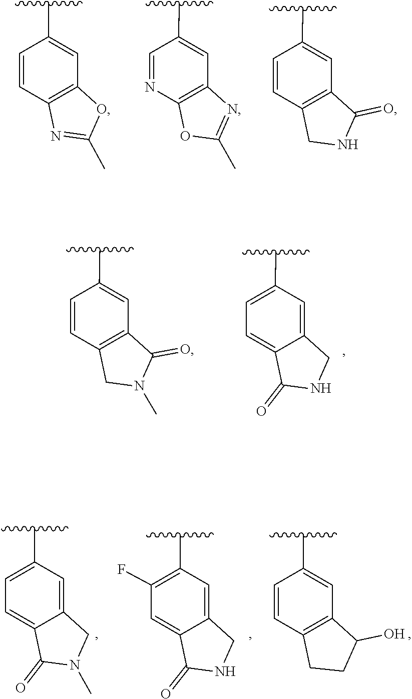

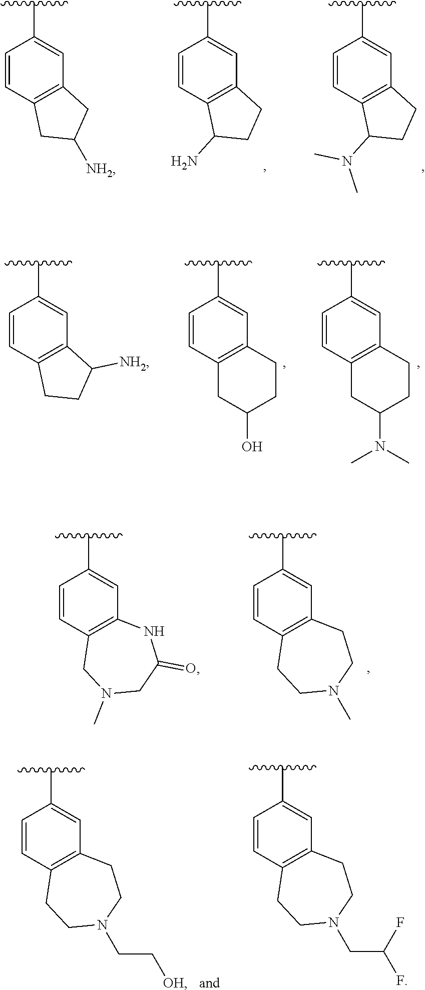

In some embodiments of any formulae described herein, each R⁵ is independently selected from oxo, =NH, —CN, halogen, —OR, —N(R)₂, —SR, —C(O)R, —N(R)C(O)R, —(CH₂)ₓC(O)N(R)₂, —C(O)N(R)(CH₂)ₓCy, —(CH₂)ₓC(O)Cy, —OC(O)R, —C(O)OR, —SO₂R, —N(R)SO₂R, —N═S(O)(R)₂, —SO₂N(R)₂, —P(O)(R)₂, —(CH₂)ₓCy, —O(CH₂)ₓCy, and C₁₋₆ aliphatic, wherein C₁₋₆ aliphatic is unsubstituted or substituted with one or more halogen, —CN, —N(R)C(O)R, —N(R)₂, or —OR. In some embodiments, each R⁵ is independently selected from oxo, =NH, —CN, halogen, —OR, —N(R)₂, —SR, —C(O)R, —N(R)C(O)R, —(CH₂)ₓC(O)N(R)₂, —OC(O)R, —C(O)OR, —SO₂R, —N(R)SO₂R, —SO₂N(R)₂, —(CH₂)ₓCy, and C₁₋₆ aliphatic, wherein C₁₋₆ aliphatic is unsubstituted or substituted with one or more halogen, or —OR. In some embodiments, each R⁵ is independently selected from —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, —(CH₂)ₓCy, and optionally substituted C₁₋₆ aliphatic (e.g., C₁₋₆ aliphatic substituted with one or more substituents selected from halogen, —OR, —C(O)NR₂, and —N(R)₂). In some embodiments, each R⁵ is independently selected from oxo, =NH, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, —(CH₂)ₓCy, and optionally substituted C₁₋₆ alkyl (e.g., C₁₋₆ alkyl substituted with one or more substituents selected from halogen —OR, —C(O)NR₂, and —N(R)₂). In some embodiments, each R⁵ is independently selected from oxo, —CN, halogen, —N(R)₂, —C(O)R, —C(O)N(R)₂, —SO₂R, —SO₂N(R)₂, —(CH₂)ₓCy, and optionally substituted C₁₋₆ aliphatic (e.g., C₁₋₆ aliphatic substituted with one or more substituents selected from halogen, —OR, —C(O)NR₂, and —N(R)₂). In some embodiments, each R⁵ is independently selected from oxo, —CN, halogen, —OR, —N(R)₂, —C(O)R, —(CH₂)ₓC(O)N(R)₂, —C(O)N(R)(CH₂)ₓCy, —(CH₂)ₓC(O)Cy, —SO₂R, —N═S(O)(R)₂, —SO₂N(R)₂, —P(O)(R)₂, —(CH₂)ₓCy, —O(CH₂)ₓCy, and C₁₋₆ aliphatic, wherein C₁₋₆ aliphatic is unsubstituted or substituted with one or more halogen, —CN, —N(R)C(O)R, —N(R)₂, or —OR. In some embodiments, each R⁵ is independently selected from oxo, —CN, halogen, —NH₂, —N(C₁₋₄ alkyl)₂, —C(O)(C₁₋₄ alkyl), —C(O)NH₂, —SO₂(C₁₋₄ alkyl), —SO₂NH₂, —SO₂N(H)(C₁₋₄ alkyl), —CH₂(3- to 6-membered carbocyclic ring optionally substituted with one or more halogen), 4- to 6-membered heterocyclic ring having 1-2 heteroatoms independently selected from nitrogen, oxygen, and sulfur, and C₁₋₆ alkyl optionally substituted with one or more halogen, —OR^{∘}, and —C(O)NR^{∘}₂. In some such embodiments R^{∘} is hydrogen. Accordingly, in some embodiments, each R⁵ is independently selected from oxo, —CN, halogen, —NH₂, —N(C₁₋₄ alkyl)₂, —C(O)(C₁₋₄ alkyl), —C(O)NH₂, —SO₂(C₁₋₄ alkyl), —SO₂NH₂, —SO₂N(H)(C₁₋₄ alkyl), —CH₂(3- to 6-membered carbocyclic ring optionally substituted with one or more halogen), 4- to 6-membered heterocyclic ring having 1-2 heteroatoms independently selected from nitrogen, oxygen, and sulfur, and C₁₋₆ alkyl optionally substituted with one or more halogen, —OH, and —CO₂NH₂.

In some embodiments of any formulae described herein, R⁵ is oxo. In some embodiments, R⁵ is =NH. In some embodiments, R⁵ is —CN. In some embodiments, R⁵ is halogen (e.g., fluoro or chloro). In some embodiments, R⁵ is fluoro. In some embodiments, R⁵ is chloro. In some embodiments, R⁵ is —OR. In some embodiments, R⁵ is —O(C₁₋₄ alkyl optionally substituted with one or more —N(C₁₋₄ alkyl)₂). In some embodiments, R⁵ is —N(R)₂. In some embodiments, R⁵ is —NH₂. In some embodiments, R⁵ is —N(C₁₋₄ alkyl)₂. In some embodiments, R⁵ is —SR. In some embodiments, R⁵ is —C(O)R. In some embodiments, R⁵ is —C(O)(C₁₋₄ alkyl). In some embodiments, R⁵ is —N(R)C(O)R. In some embodiments, R⁵ is —(CH₂)ₓC(O)N(R)₂. In some embodiments, R⁵ is —(CH₂)ₓC(O)NH₂, —(CH₂)ₓC(O)NH(C₁₋₄ alkyl), —(CH₂)ₓC(O)NH(C₁₋₄ haloalkyl), —(CH₂)ₓC(O)N(C₁₋₄ alkyl)₂, or —(CH₂)ₓC(O)N(C₁₋₄ haloalkyl)₂. In some embodiments, R⁵ is —C(O)N(R)₂. In some embodiments, R⁵ is —C(O)NH₂, —C(O)NH(C₁₋₄ alkyl), —C(O)NH(C₁₋₄ haloalkyl), —C(O)N(C₁₋₄ alkyl)₂, or —C(O)N(C₁₋₄ haloalkyl)₂. In some embodiments, R⁵ is —C(O)N(R)(CH₂)ₓCy (e.g., —C(O)NH(CH₂)ₓCy). In some embodiments, R⁵ is —(CH₂)ₓC(O)Cy. In some embodiments, R⁵ is —OC(O)R. In some embodiments, R⁵ is —C(O)OR. In some embodiments, R⁵ is —SO₂R. In some embodiments, R⁵ is —SO₂(C₁₋₄ alkyl). In some embodiments, R⁵ is —N(R)SO₂R. In some embodiments, R⁵ is —N═S(O)(R)₂. In some embodiments, R⁵ is —SO₂N(R)₂. In some embodiments, R⁵ is —SO₂NH₂ or —SO₂NH(C₁₋₄ alkyl). In some embodiments, R⁵ is —P(O)R₂. In some embodiments, R⁵ is —(CH₂)ₓCy. In some embodiments, R⁵ is —(CH₂)ₓCy, wherein Cy is 3- to 6-membered carbocyclic ring optionally substituted with one or more halogen. In some embodiments, R⁵ is —(CH₂)ₓCy, wherein Cy is 4- to 6-membered heterocyclic ring having 1-2 heteroatoms independently selected from nitrogen, oxygen, and sulfur. In some embodiments, R⁵ is —(CH₂)ₓCy, and x is 0. In some embodiments, R⁵ is —(CH₂)ₓCy, wherein x is 0 and Cy is 3- to 6-membered carbocyclic ring optionally substituted with one or more halogen. In some embodiments, R⁵ is —(CH₂)ₓCy, wherein x is 0 and Cy is 4- to 6-membered heterocyclic ring having 1-2 heteroatoms independently selected from nitrogen, oxygen, and sulfur substituted with 0-2 instances of R⁶. In some embodiments, R⁵ is —O(CH₂)ₓCy. In some embodiments, R⁵ is optionally substituted C₁₋₆ aliphatic (e.g., C₁₋₆ aliphatic substituted with one or more substituents selected from halogen, —OR, —CN, —N(R)C(O)R, —C(O)NR₂, and —N(R)₂). In some embodiments, R⁵ is C₁₋₆ aliphatic optionally substituted with one or more halogen, —OR^{∘} (e.g., —OH), and —C(O)NR^{∘}₂ (e.g., —CO₂NH₂). In some embodiments, R⁵ is optionally substituted C₁₋₆ alkyl (e.g., C₁₋₆ alkyl substituted with one or more substituents selected from halogen, —OR, —CN, —N(R)C(O)R, —C(O)NR₂, and —N(R)₂). In some embodiments, R⁵ is C₁₋₆ alkyl optionally substituted with one or more halogen, —OH, and —CO₂NH₂.

In some embodiments of any formulae described herein, each R⁵ is independently selected from oxo, —CN, fluoro, chloro, —NH₂, —N(CH₃)₂, —OCH₃, —C(O)CH₃, —C(O)N(CH₃)₂, —C(O)N(H)CH₃, —CH₂C(O)N(H)CH₃, —CH₂C(O)N(CH₃)₂, —C(O)N(H)CH₂CH₂N(CH₃)₂, —CO₂NH₂, —SO₂CH₃, —SO₂NH₂, —SO₂N(H)CH₃, —N═S(O)(CH₃)₂, —P(O)(CH₃)₂, —CH₃, —CH₂F, —CHF₂, —CH₂CHF₂, —CH₂OH, —CH₂CH₂OH, —CH₂CH₂OCH₃, —CH₂C(CH₃)₂OH, —C(CH₃)₂CH₂OH, —C(CH₃)₂OH, —C(CH₃)₂NH₂, —C(CH₃)₂CN, —CH₂N(CH₃)₂, —CH₂CH₂N(CH₃)₂, —CH₂C(CH₃)₂NH₂, —CH₂N(H)C(O)CH₃, —OCH₂CH₂N(CH₃)₂, —CH₂CH₂N(CH₃)₂, —C(O)N(CH₃)₂, —CH₂C(O)NH₂, —CH₂C(O)N(H)(CH₃), —CH₂C(O)N(CH₃)₂, —CH(CH₃)₂, —CH(CF₃)N(H)CH₃, —CH(CF₃)N(CH₃)₂, —C(O)N(H)CH₃, —C(O)N(H)CH₂CHF₂, —OCH₂CH₂N(CH₃)₂, —C(O)N(H)CH₂CH₂N(CH₃)₂, —C(O)N(CH₃)CH₂CH₂N(CH₃)₂,

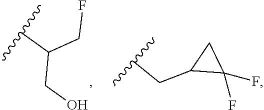 [SMILES: *C(CO)CF ; *CC1CC1(F)F]

cyclopropane, cyclobutane,

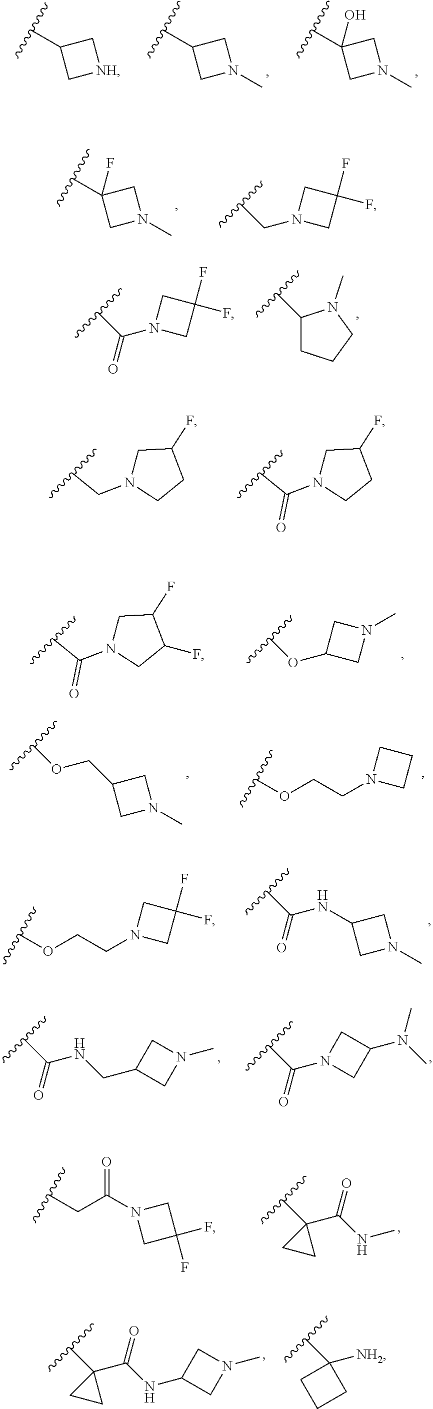

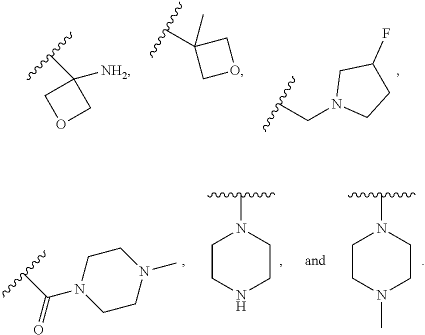

In some embodiments, each R⁵ is independently selected from oxo, —CN, fluoro, chloro, —NH₂, —N(CH₃)₂, —OCH₃, —C(O)CH₃, —C(O)N(CH₃)₂, —C(O)N(H)CH₃, —CH₂C(O)N(H)CH₃, —CH₂C(O)N(CH₃)₂, —C(O)N(H)CH₂CH₂N(CH₃)₂, —CO₂NH₂, —SO₂CH₃, —SO₂NH₂, —SO₂N(H)CH₃, —N=S(O)(CH₃)₂, —P(O)(CH₃)₂, —CH₃, —CH₂F, —CHF₂, —CH₂CHF₂, —CH₂OH, —CH₂CH₂OH, —CH₂CH₂OCH₃, —CH₂C(CH₃)₂OH, —C(CH₃)₂CH₂OH, —C(CH₃)₂OH, —C(CH₃)₂NH₂, —C(CH₃)₂CN, —CH₂N(CH₃)₂, —CH₂CH₂N(CH₃)₂, —CH₂C(CH₃)₂NH₂, —OCH₂CH₂N(CH₃)₂, —CH₂CH₂N(CH₃)₂, —CH₂C(O)NH₂, —CH(CH₃)₂, —CH(CF₃)N(H)CH₃, and —CH(CF₃)N(CH₃)₂.

In some embodiments of any formulae described herein, each R^{5′} is independently selected from oxo, =NH, —CN, halogen, —OR, —N(R)₂, —SR, —C(O)R, —N(R)C(O)R, —(CH₂)ₓC(O)N(R)₂, —(CH₂)ₓC(O)Cy, —OC(O)R, —C(O)OR, —SO₂R, —N(R)SO₂R, —N═S(O)(R)₂, —SO₂N(R)₂, —P(O)(R)₂, —(CH₂)ₓCy, —O(CH₂)ₓCy, and C₁₋₆ aliphatic, wherein C₁₋₆ aliphatic is unsubstituted or substituted with one or more halogen, —CN, —N(R)C(O)R, —N(R)₂, or —OR. In some embodiments, each R^{5′} is independently selected from oxo, =NH, —CN, halogen. —OR, —N(R)₂. —SR, —C(O)R, —N(R)C(O)R, —(CH₂)ₓC(O)N(R)₂, —OC(O)R, —C(O)OR, —SO₂R, —N(R)SO₂R, —SO₂N(R)₂, —(CH₂)ₓCy, and C₁₋₆ aliphatic, wherein C₁₋₆ aliphatic is unsubstituted or substituted with one or more halogen and —OR. In some embodiments, each R^{5′} is independently selected from —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, —(CH₂)ₓCy, and optionally substituted C₁₋₆ aliphatic (e.g., C₁₋₆ aliphatic substituted with one or more substituents selected from halogen, —OR, —C(O)NR₂, and —N(R)₂). In some embodiments, each R^{5′} is independently selected from oxo, =NH, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, —(CH₂)ₓCy, and optionally substituted C₁₋₆ alkyl (e.g., C₁₋₆ alkyl substituted with one or more substituents selected from halogen —OR, —C(O)NR₂, and —N(R)₂). In some embodiments, each R^{5′} is independently selected from oxo, =NH, —OR, —N(R)₂, and optionally substituted C₁₋₆ aliphatic (e.g., C₁₋₆ aliphatic substituted with one or more substituents selected from halogen, —OR, —C(O)NR₂, and —N(R)₂). In some embodiments, each R^{5′} is independently selected from oxo, =NH, halogen, —OR, —N(R)₂, and C₁₋₆ aliphatic, wherein C₁₋₆ aliphatic is unsubstituted or substituted with one or more halogen, —N(R)₂, or —OR. In some embodiments, each R^{5′} is independently selected from oxo, =NH, —OH, —NH₂, —N(C₁₋₄ alkyl)₂, and C₁₋₆ alkyl optionally substituted with one or more —OR^{∘} (e.g., —OH).

In some embodiments of any formulae described herein, R^{5′} is oxo. In some embodiments, R^{5′} is =NH. In some embodiments, R^{5′} is —CN. In some embodiments, R^{5′} is halogen (e.g., fluoro or chloro). In some embodiments, R^{5′} is fluoro. In some embodiments, R^{5′} is —OR (e.g., —OH or —OCH₃). In some embodiments, R^{5′} is —N(R)₂. In some embodiments, R^{5′} is —NH₂. In some embodiments, R⁵¹ is —N(C₁₋₄ alkyl)₂. In some embodiments, R^{5′} is —SR. In some embodiments, R^{5′} is —C(O)R. In some embodiments, R^{5′} is —N(R)C(O)R. In some embodiments, R^{5′} is —(CH₂)ₓC(O)N(R)₂. In some embodiments, R^{5′} is —C(O)N(R)₂. In some embodiments, R^{5′} is —C(O)N(R)(CH₂)ₓCy. In some embodiments, R^{5′} is —(CH₂)ₓC(O)Cy. In some embodiments, R^{5′} is —OC(O)R. In some embodiments, R^{5′} is —C(O)OR. In some embodiments, R^{5′} is —SO₂R. In some embodiments, R⁵ is —N(R)SO₂R. In some embodiments, R^{5′} is —N=S(O)(R)₂. In some embodiments, R^{5′}, is —SO₂N(R)₂. In some embodiments, R^{5′} is —P(O)R₂. In some embodiments, R^{5′} is —(CH₂)ₓCy. In some embodiments, R⁵ is —(CH₂)ₓCy, wherein Cy is 3- to 6-membered carbocyclic ring optionally substituted with one or more halogen. In some embodiments, R⁵ is —(CH₂)ₓCy, wherein Cy is 4- to 6-membered heterocyclic ring having 1-2 heteroatoms independently selected from nitrogen, oxygen, and sulfur. In some embodiments, R⁵ is —(CH₂)ₓCy, and x is 0. In some embodiments, R⁵ is —(CH₂)ₓCy, wherein x is 0 and Cy is 3- to 6-membered carbocyclic ring optionally substituted with one or more halogen. In some embodiments, R⁵ is —(CH₂)ₓCy, wherein x is 0 and Cy is 4- to 6-membered heterocyclic ring having 1-2 heteroatoms independently selected from nitrogen, oxygen, and sulfur. In some embodiments, R^{5′} is —O(CH₂)ₓCy. In some embodiments, R^{5′} is optionally substituted C₁₋₆ aliphatic. In some embodiments, R^{5′} is C₁₋₆ aliphatic optionally substituted with one or more —OR^{∘} (e.g., —OH). In some embodiments, R^{5′} is C₁₋₆ aliphatic optionally substituted with one or halogens (e.g., F), —OH, or —O(C₁₋₄ alkyl). In some embodiments, R^{5′} is optionally substituted C₁₋₆ alkyl. In some embodiments, R^{5′} is C₁₋₆ alkyl optionally substituted with one or more —OH, such as —CH₂CH₂OH. In some embodiments, R^{5′} is C₁₋₆ alkyl optionally substituted with one or more halogens (e.g., F), —OH, or —O(C₁₋₄ alkyl). In some embodiments, R^{5′} is unsubstituted C₁₋₆ aliphatic. In some embodiments, R^{5′} is unsubstituted C₁₋₆ alkyl. In some embodiments, R^{5′} is methyl, ethyl, or isopropyl.

In some embodiments of any formulae described herein, each R^{5′} is independently selected from oxo, =NH, —OH, —OCH₃, —NH₂, —N(CH₃)₂, fluoro, cyclopropyl, —CH₃, —CD₃, —CH₂CHF₂, —CH₂CF₃, —CH₂CH₂CH₂F, —CH₂CH₂N(CH₃)₂, —CH₂CH₂OH, —CH₂CH₂OCH₃, and —CH(CH₃)₂. In some embodiments, each R^{5′} is independently selected from oxo, =NH, —OH, —NH₂, —N(CH₃)₂, —CH₃, —CH₂CH₂OH, and —CH(CH₃)₂.

In some embodiments of any formulae described herein, p is 0, 1, or 2. In some embodiments, p is 0 or 1. In some embodiments, p is 1 or 2. In some embodiments, p is 0. In some embodiments, p is 1. In some embodiments, p is 2. In some embodiments, p is 3.

In some embodiments of any formulae described herein, s is 0, 1, or 2. In some embodiments, s is 0 or 1. In some embodiments, s is 1 or 2. In some embodiments, s is 0. In some embodiments, s is 1. In some embodiments, s is 2. In some embodiments, s is 3.

In some embodiments of any formulae described herein, Ring D is selected from phenyl and a 5- to 6-membered heteroaryl ring having 1-3 heteroatoms independently selected from nitrogen, oxygen, and sulfur. In some embodiments, Ring D is selected from phenyl and a 5- to 6-membered heteroaryl ring having 1-3 heteroatoms independently selected from nitrogen, oxygen, and sulfur, wherein Ring D is fused to Ring F.

In some embodiments of any formulae described herein, Ring D is not fused to Ring F. In some embodiments, Ring D is fused to Ring F. In some embodiments, when Ring D is fused to Ring F, q is 0.

In some embodiments of any formulae described herein, Ring D is phenyl.

In some embodiments of any formulae described herein, Ring D is a 5- to 6-membered heteroaryl ring having 1-3 heteroatoms independently selected from nitrogen, oxygen, and sulfur. In some embodiments, Ring D is a 5-membered heteroaryl ring having 1-3 heteroatoms independently selected from nitrogen, oxygen, and sulfur. In some embodiments, Ring D is a pyrazole, imidazole, triazole, oxazole, or thiazole. In some embodiments, Ring D is a 6-membered heteroaryl ring having 1-3 nitrogen atoms. In some embodiments, Ring D is a pyridine, pyridone, pyridazinone, or pyrazinone.

In some embodiments of any formulae described herein, Ring D is not fused to Ring F, and Ring D, substituted with r instances of R² and q instances of R¹, is selected from

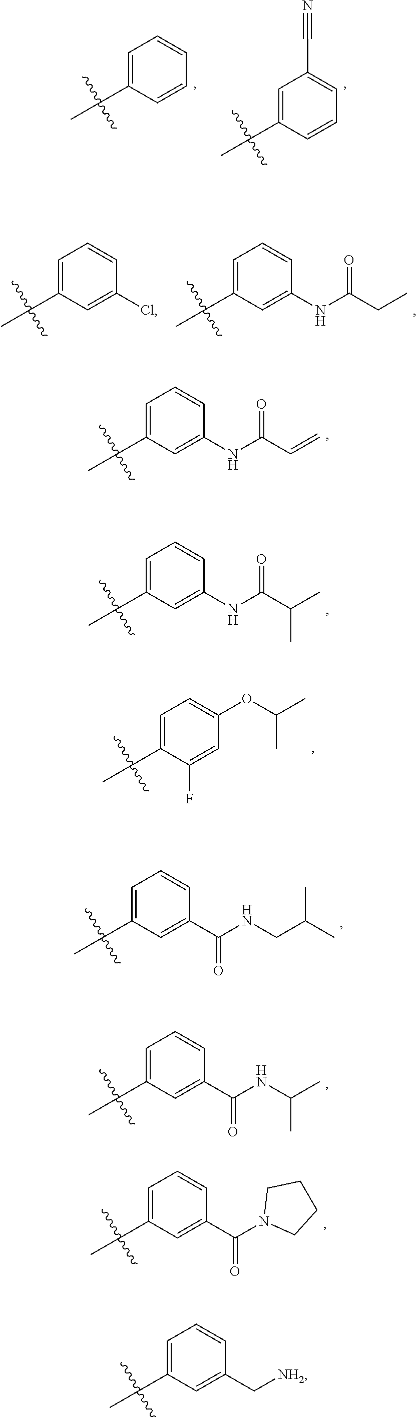

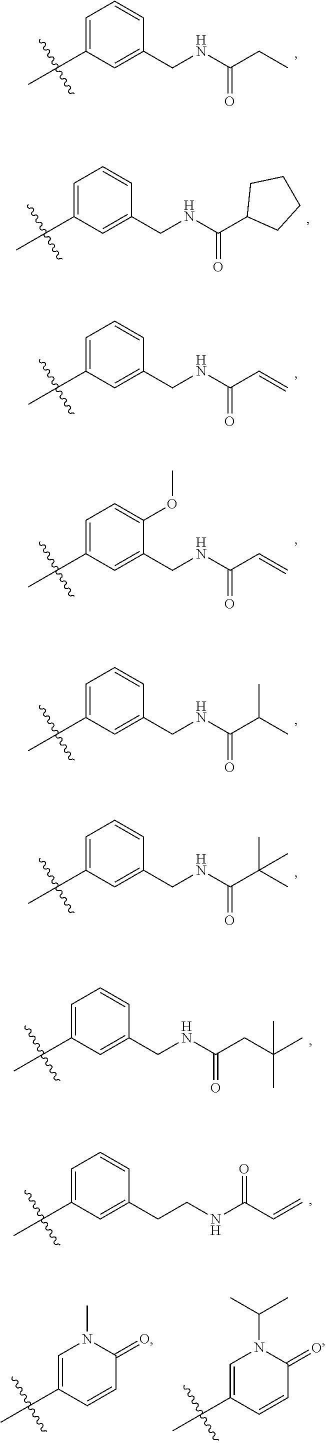

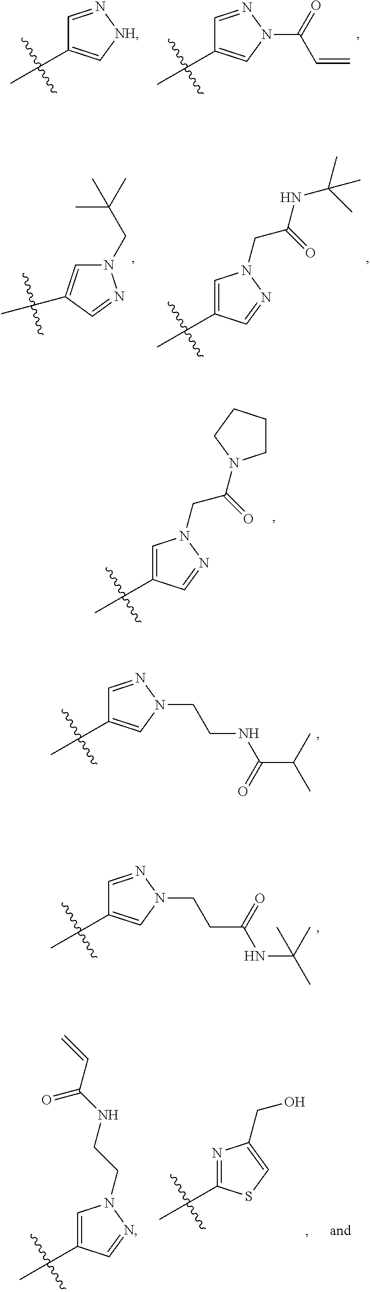

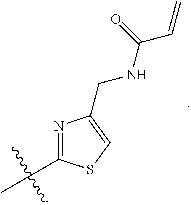 [SMILES: *c1nc(CNC(=O)C=C)cs1]

In some embodiments of any formulae described herein, Ring F is selected from phenyl, a 5- to 6-membered saturated or partially unsaturated carbocyclic ring, a 5- to 7-membered saturated or partially unsaturated heterocyclic ring having 1-2 heteroatoms independently selected from nitrogen, oxygen, and sulfur, and a 5- to 6-membered heteroaryl ring having 1-3 heteroatoms independently selected from nitrogen, oxygen, and sulfur, wherein Ring F is substituted by u instances of -L-W and y instances of R^{2′}. In some embodiments, Ring F is selected from phenyl, a 5- to 6-membered saturated or partially unsaturated carbocyclic ring, a 5- to 6-membered saturated or partially unsaturated heterocyclic ring having 1-2 heteroatoms independently selected from nitrogen, oxygen, and sulfur, and a 5- to 6-membered heteroaryl ring having 1-3 heteroatoms independently selected from nitrogen, oxygen, and sulfur, wherein Ring F is substituted by u instances of -L-W.

In some embodiments of any formulae described herein, Ring F is phenyl substituted by u instances of -L-W and y instances of R^{2′}. In some embodiments, Ring F is phenyl substituted by u instances of -L-W.

In some embodiments of any formulae described herein, Ring F is a 5- to 6-membered carbocyclic ring substituted by u instances of -L-W and y instances of R^{2′}. In some embodiments, Ring F is a 5- to 6-membered carbocyclic ring substituted by u instances of -L-W. In some embodiments, Ring F is a 5- to 6-membered saturated or partially unsaturated carbocyclic ring substituted by u instances of -L-W. In some embodiments, Ring F is a 5-membered carbocyclic ring substituted by u instances of -L-W. In some embodiments, Ring F is a cyclopentane substituted by u instances of -L-W. In some embodiments, Ring F is a 6-membered carbocyclic ring substituted by u instances of -L-W.

In some embodiments of any formulae described herein, Ring F is a 5- to 7-membered heterocyclic ring having 1-2 heteroatoms independently selected from nitrogen, oxygen, and sulfur, wherein Ring F is substituted by u instances of -L-W and y instances of R². In some embodiments, Ring F is a 5- to 6-membered heterocyclic ring having 1-2 heteroatoms independently selected from nitrogen, oxygen, and sulfur, wherein Ring F is substituted by u instances of -L-W. In some embodiments, Ring F is a 5- to 7-membered saturated or partially unsaturated heterocyclic ring having 1-2 heteroatoms independently selected from nitrogen, oxygen, and sulfur, wherein Ring F is substituted by u instances of -L-W and y instances of R^{2′}. In some embodiments, Ring F is a 5- to 6-membered saturated or partially unsaturated heterocyclic ring having 1-2 heteroatoms independently selected from nitrogen, oxygen, and sulfur, wherein Ring F is substituted by u instances of -L-W. In some embodiments, Ring F is 5-membered heterocyclic ring having 1-2 heteroatoms independently selected from nitrogen, oxygen, and sulfur, wherein Ring F is substituted by u instances of -L-W. In some embodiments, Ring F is a pyrrolidine substituted by u instances of -L-W. In some embodiments, Ring F is a 6-membered heterocyclic ring having 1-2 heteroatoms independently selected from nitrogen, oxygen, and sulfur, wherein Ring F is substituted by u instances of -L-W and y instances of R^{2′}. In some embodiments, Ring F is 6-membered heterocyclic ring having 1-2 heteroatoms independently selected from nitrogen, oxygen, and sulfur, wherein Ring F is substituted by u instances of -L-W. In some embodiments, Ring F is a piperidine or piperazine, each substituted by u instances of -L-W and y instances of R^{2′}. In some embodiments, Ring F is a piperidine or piperazine, each substituted by u instances of -L-W.

In some embodiments of any formulae described herein, Ring F is a 5- to 6-membered heteroaryl ring having 1-3 heteroatoms independently selected from nitrogen, oxygen, and sulfur, wherein Ring F is substituted by u instances of -L-W and y instances of RU. In some embodiments, Ring F is a 5- to 6-membered heteroaryl ring having 1-3 heteroatoms independently selected from nitrogen, oxygen, and sulfur, wherein Ring F is substituted by u instances of -L-W. In some embodiments, Ring F is a 5-membered heteroaryl ring having 1-3 heteroatoms independently selected from nitrogen, oxygen, and sulfur, wherein Ring F is substituted by u instances of -L-W. In some embodiments, Ring F is a pyrrole substituted by u instances of -L-W. In some embodiments, Ring F is a 6-membered heteroaryl ring having 1-3 nitrogen atoms substituted by u instances of -L-W.

In some embodiments of any formulae described herein, u is 0. In some embodiments, u is 1. In some embodiments, when u is 1, then q is 0.

In some embodiments of any formulae described herein, Ring D is fused to Ring F, q is 0, and

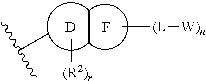

is selected from

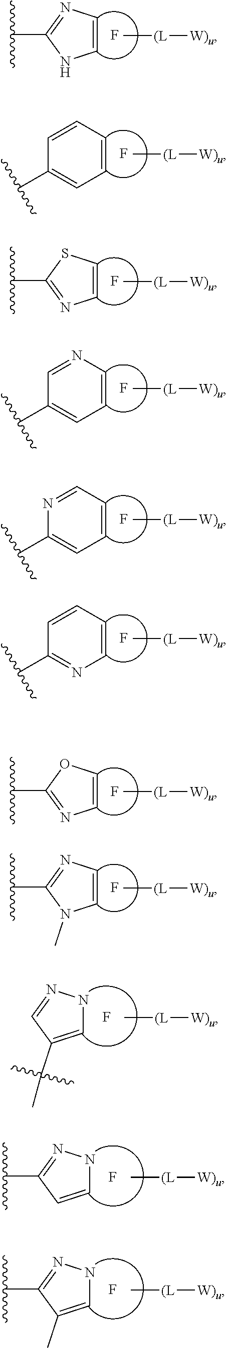

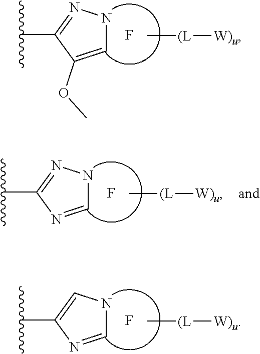

In some embodiments of any formulae described herein, Ring D is fused to Ring F, q is 0, and

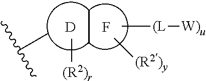

is selected from

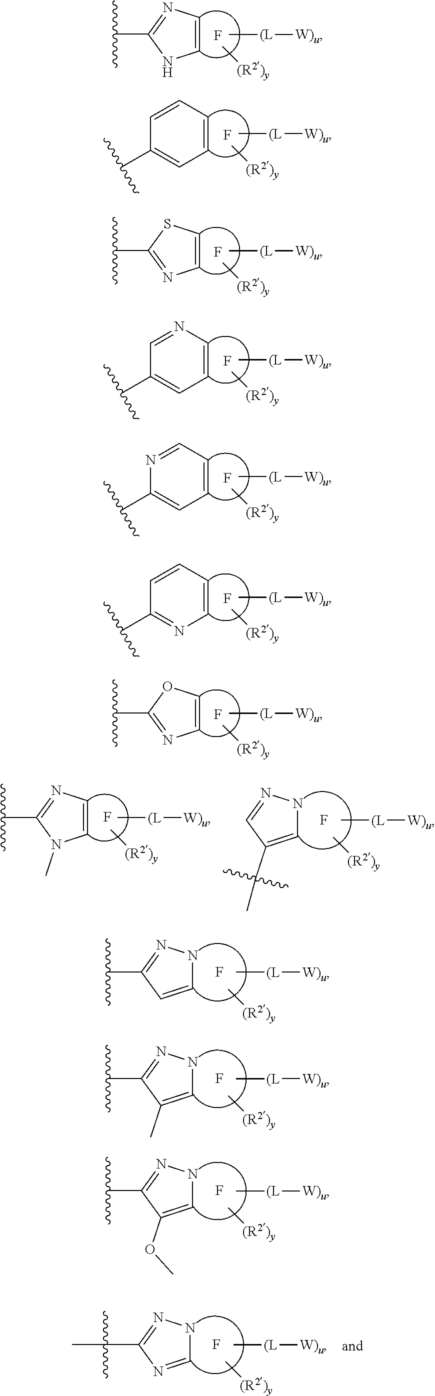

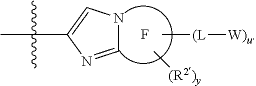

In some embodiments of any formulae described herein, Ring D is fused to Ring F, and Ring F, substituted with u instances of -L-W and y instances of R^{2′}, is selected from

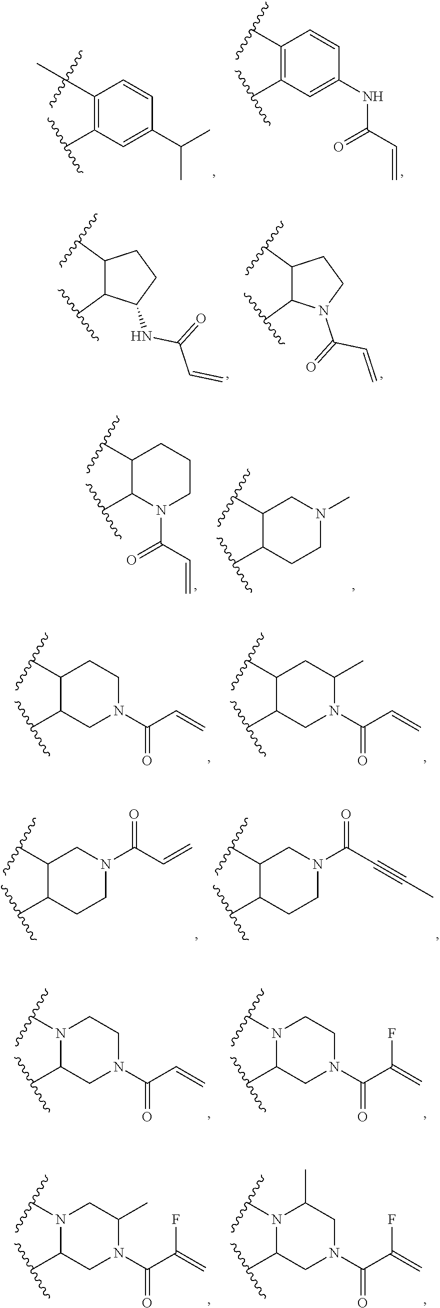

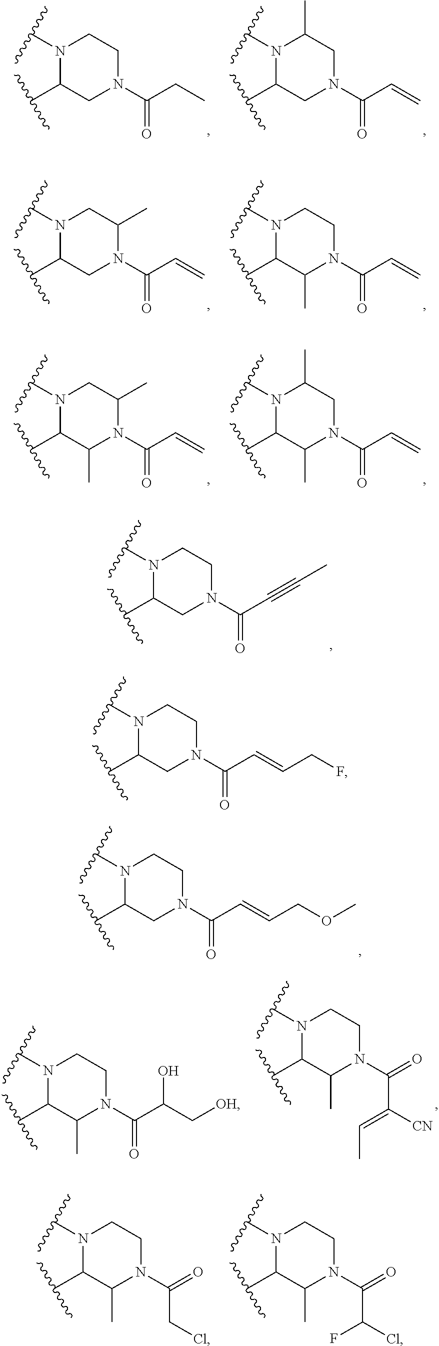

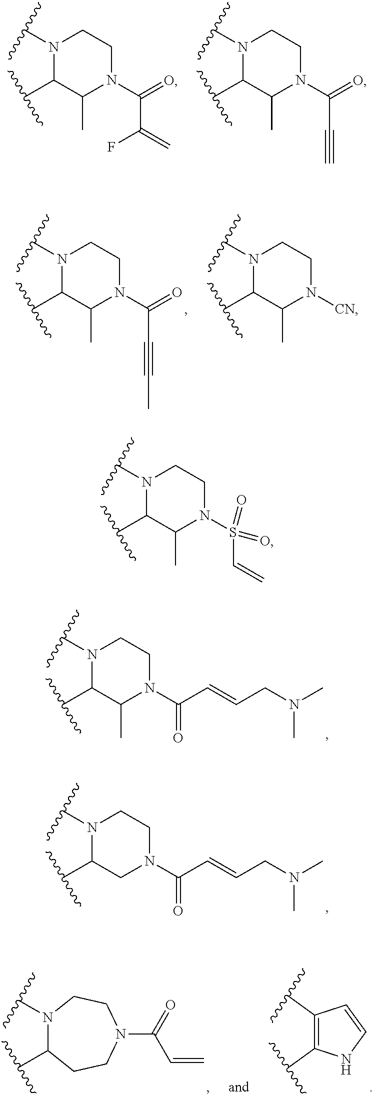

In some embodiments of any formulae described herein, Ring D is fused to Ring F, q is 0, and

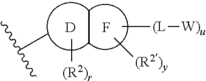

is selected from

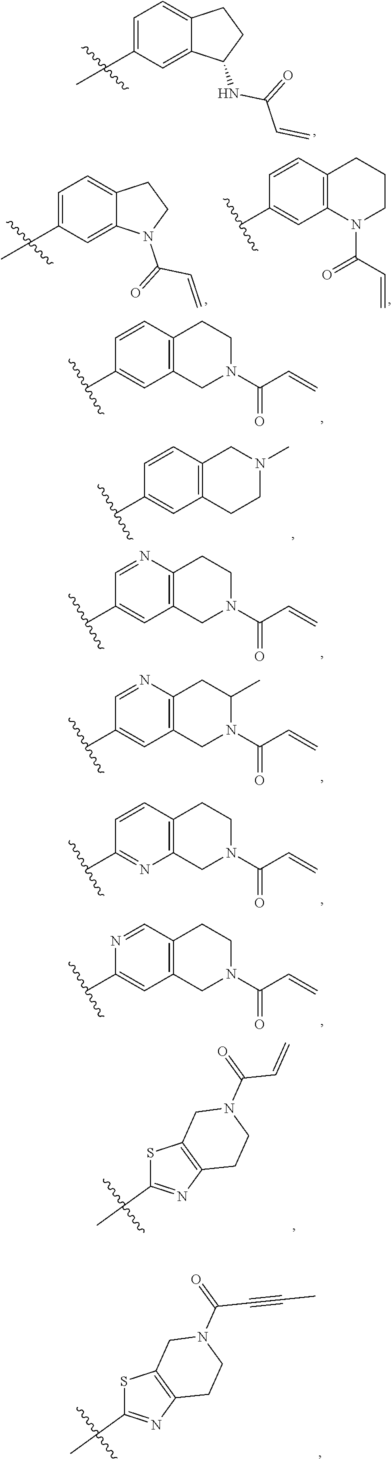

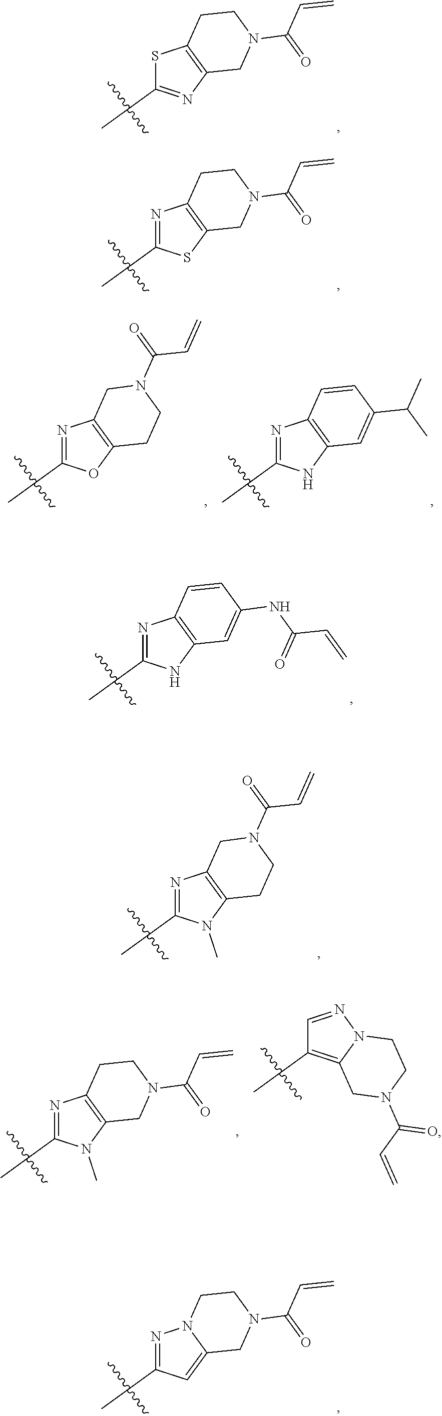

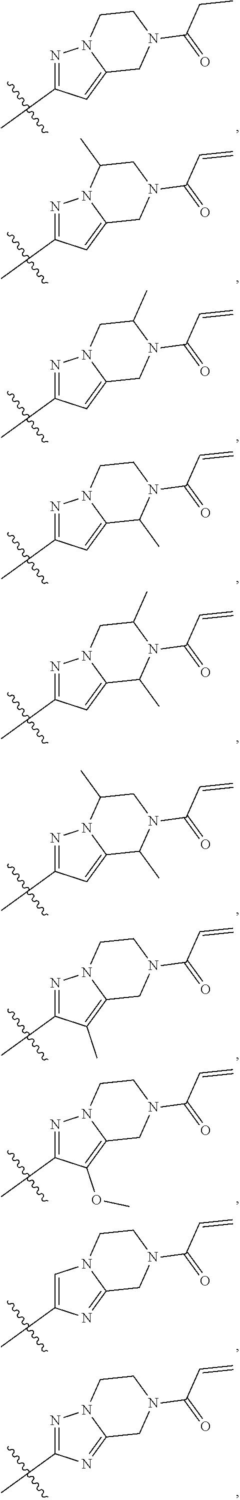

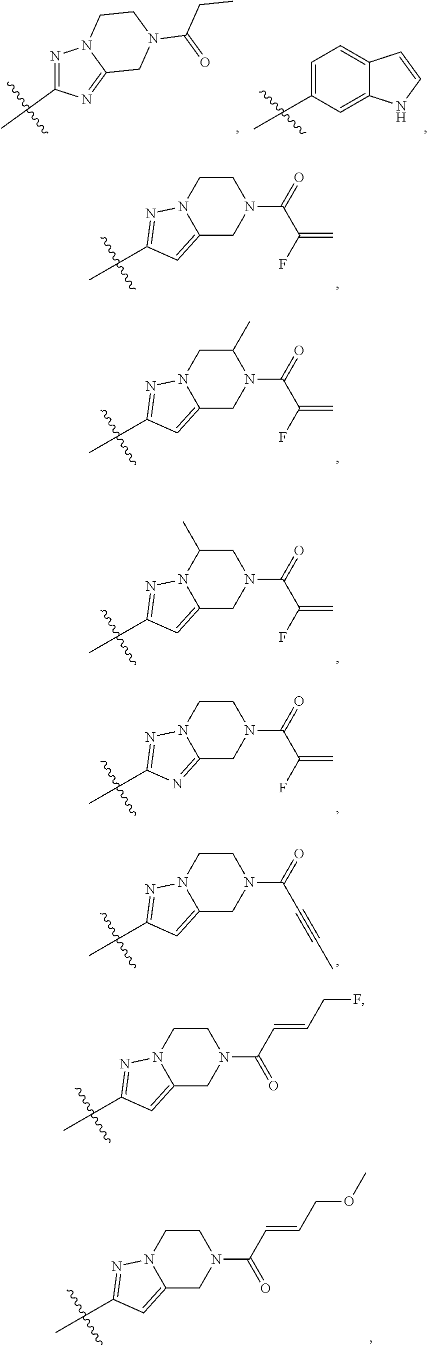

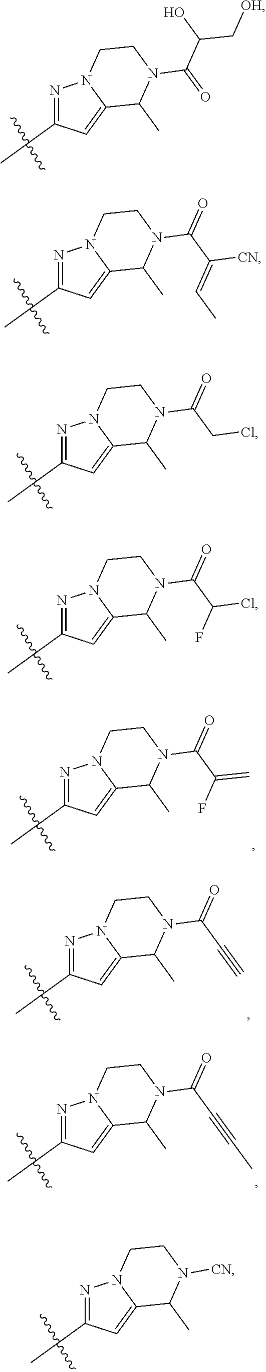

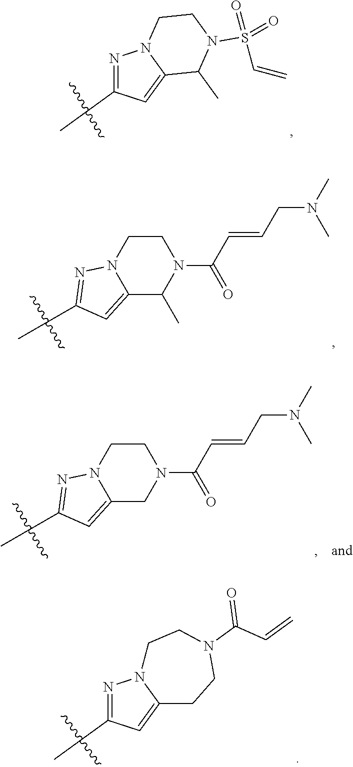 [SMILES: *c1cc2n(n1)CCN(S(=O)(=O)C=C)C2C ; *c1cc2n(n1)CCN(C(=O)/C=C/CN(C)C)C2C ; *c1cc2n(n1)CCN(C(=O)/C=C/CN(C)C)C2 ; *c1cc2n(n1)CCN(C(=O)C=C)CC2]

In some embodiments of any formulae described herein, R¹ is -L-W or Ring D′. In some embodiments, R¹ is Ring D′ or a bivalent C₁₋₆ aliphatic chain substituted with Ring D′. In some embodiments, R¹ is Ring D′ or —CH₂—Ring D′. In some embodiments, R¹ is -L-W. In some embodiments, R¹ is Ring D′. In some embodiments, R¹ is a bivalent C₁₋₆ aliphatic chain substituted with Ring D′. In some embodiments, R¹ is —CH₂—Ring D′.

In some embodiments of any formulae described herein, R¹ is selected from —CH₃, —CH(CH₃)₂, —CH₂C(CH₃)₃, —CH₂NH₂, —CH₂OH, —OCH(CH₃)₂,

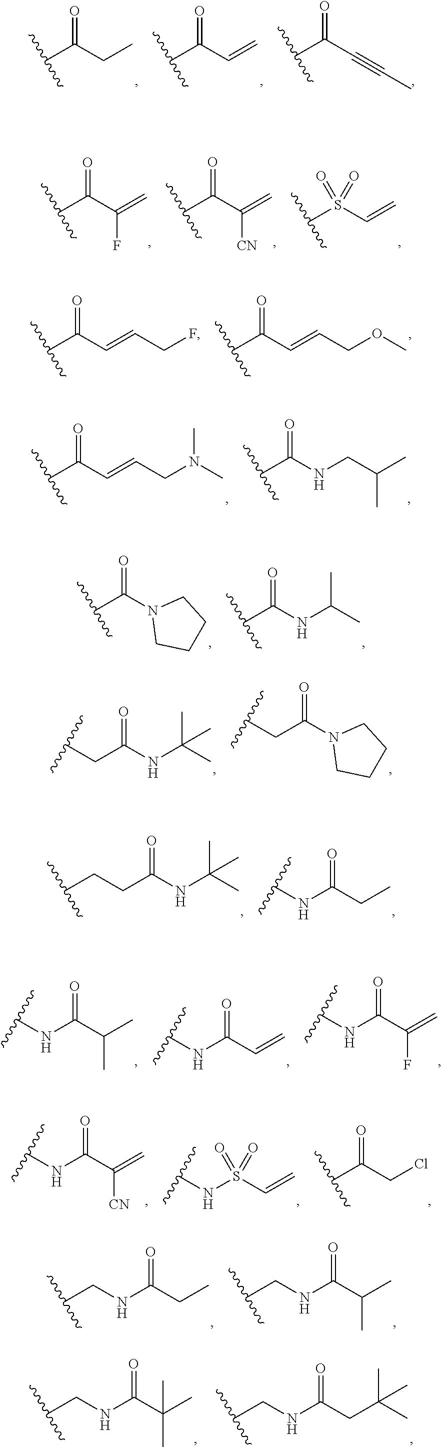

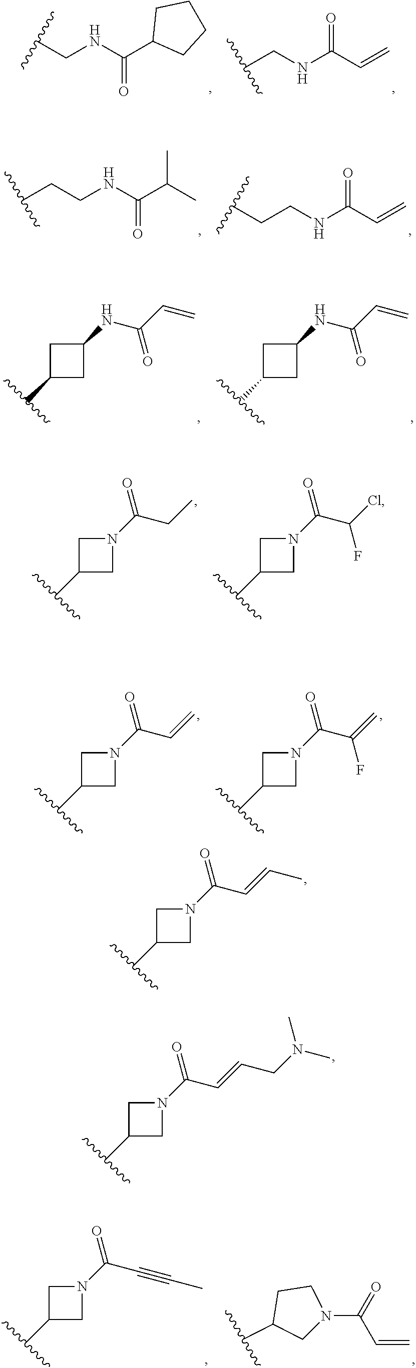

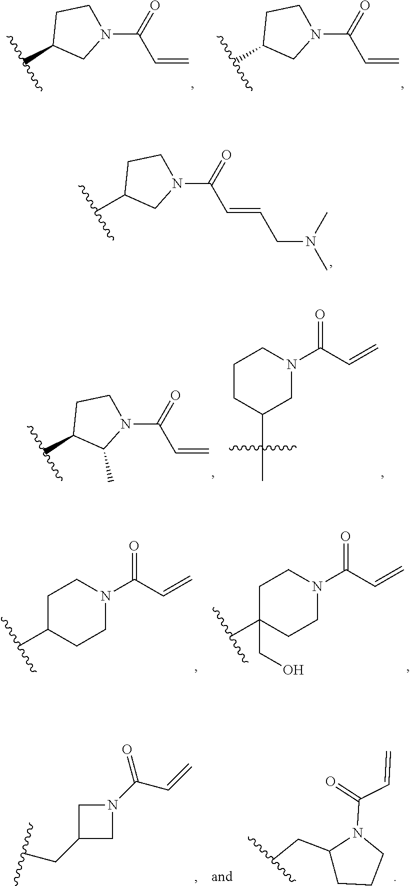

In some embodiments of any formulae described herein, each L is independently a bivalent straight or branched C₁₋₆ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₄ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.

In some embodiments of any formulae described herein, each L is independently a bivalent straight or branched C₁₋₈ aliphatic chain, wherein one or two methylene units of the aliphatic chain are independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₆ aliphatic chain, wherein one or two methylene units of the aliphatic chain are independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₄ aliphatic chain, wherein one or two methylene units of the aliphatic chain are independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.

In some embodiments of any formulae described herein, each L is independently a bivalent straight or branched C₁₋₈ aliphatic chain, wherein one or two methylene units of the aliphatic chain are independently replaced by a group selected from —N(R)—, —O—, C(O)—, —C(O)N(R)—, and —N(R)C(O)—. In some embodiments, each L is independently a bivalent straight or branched C₁₋₆ aliphatic chain, wherein one or two methylene units of the aliphatic chain are independently replaced by a group selected from —N(R)—, —O—, C(O)—, —C(O)N(R)—, and —N(R)C(O)—. In some embodiments, each L is independently a bivalent straight or branched C₁₋₄ aliphatic chain, wherein one or two methylene units of the aliphatic chain are independently replaced by a group selected from —N(R)—, —O—, C(O)—, —C(O)N(R)—, and —N(R)C(O)—.

In some embodiments of any formulae described herein, each L is independently a bivalent straight or branched C₁₋₈ alkyl chain, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₆ alkyl chain, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—. —C(O)O—, —OC(O)—, —SO₂N(R)—, and —N(R)SO₂—. In some embodiments, each L is independently a bivalent straight or branched C₁₋₄ alkyl chain, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.

In some embodiments of any formulae described herein, each L is independently a bivalent straight or branched C₁₋₈ aliphatic chain having one or more units of unsaturation, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₆ aliphatic chain having one or more units of unsaturation, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₄ aliphatic chain having one or more units of unsaturation, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.

In some embodiments of any formulae described herein, each L is independently a bivalent straight or branched C₁₋₈ aliphatic chain having one double bond, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₆ aliphatic chain having one double bond, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₄ aliphatic chain having one double bond, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.

In some embodiments of any formulae described herein, each L is independently a bivalent straight or branched C₁₋₈ aliphatic chain having one triple bond, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₆ aliphatic chain having one triple bond, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—. In some embodiments, each L is independently a bivalent straight or branched C₁₋₄ aliphatic chain having one triple bond, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.

In some embodiments of any formulae described herein, each L is independently a bivalent straight or branched C₁₋₈ aliphatic chain having one double or triple bond, wherein one or more methylene units of the aliphatic chain are optionally and independently replaced by a group selected from —N(R)—, —O—, C(O)—, —C(O)N(R)—, and —N(R)C(O)—. In some embodiments, each L is independently a bivalent straight or branched C₁₋₆ aliphatic chain having one double or triple bond, wherein one or more methylene units of the aliphatic chain are optionally and independently replaced by a group selected from —N(R)—, —O—, C(O)—, —C(O)N(R)—, and —N(R)C(O)—. In some embodiments, each L is independently a bivalent straight or branched C₁₋₄ aliphatic chain having one double or triple bond, wherein one or more methylene units of the aliphatic chain are optionally and independently replaced by a group selected from —N(R)—, —O—, C(O)—, —C(O)N(R)—, and —N(R)C(O)—.

In some embodiments of any formulae described herein, each L is independently selected from —N(R)C(O)CH₂CH₂—, —N(R)C(O)CH(CH₃)CH₂—, —OCH(CH₃)CH₂—, —C(O)—, —C(O)CClH—, —C(O)CClF—, —C(O)CH₂CH₂—, —C(O)CH(OH)CH(OH)—, —C(O)N(R)CH(CH₃)CH₂—, —C(O)N(R)CH₂CH(CH₃)CH₂—, —CH₂—, —CH(CH₃)CH₂—, —CH₂C(CH₃)₂CH₂—, —CH₂C(O)—, —CH₂C(O)N(R)C(CH₃)₂CH₂—, —CH₂CH₂C(O)N(R)C(CH₃)₂CH₂—, —CH₂N(R)—, —CH₂N(R)C(O)—, —CH₂N(R)C(O)CH₂CH₂—, —CH₂N(R)C(O)CH(CH₃)CH₂—, —CH₂N(R)C(O)C(CH₃)₂CH₂—, —CH₂N(R)C(O)CH₂C(CH₃)₂CH₂—, —CH₂CH₂N(R)C(O)CH(CH₃)CH₂—, and —CH₂O—. In some embodiments, each L is independently selected from —N(H)C(O)CH₂CH₂—, —N(H)C(O)CH(CH₃)CH₂—, —OCH(CH₃)CH₂—, —C(O)—, —C(O)CClH—, —C(O)CClF— —C(O)CH₂CH₂—, —C(O)CH(OH)CH(OH)—, —C(O)N(H)CH(CH₃)CH₂—, —C(O)N(H)CH₂CH(CH₃)CH₂—, —CH₂—, —CH(CH₃)CH₂—, —CH₂C(CH₃)₂CH₂—, —CH₂C(O)—. —CH₂C(O)N(H)C(CH₃)₂CH₂—, —CH₂CH₂C(O)N(H)C(CH₃)₂CH₂—, —CH₂N(H)—, —CH₂N(H)C(O)—, —CH₂N(H)C(O)CH₂CH₂—, —CH₂N(H)C(O)CH(CH₃)CH₂—, —CH₂N(H)C(O)C(CH₃)₂CH₂—, —CH₂N(H)C(O)CH₂C(CH₃)₂CH₂—, —CH₂CH₂N(H)C(O)CH(CH₃)CH₂—, and —CH₂O—.

In some embodiments of any formulae described herein, each L is independently selected from —N(R)C(O)CH₂CH₂—, —N(R)C(O)CH(CH₃)CH₂—, —OCH(CH₃)CH₂—, —C(O)—, —C(O)CH₂CH₂—, —C(O)CH(OH)CH(OH)—, —C(O)N(R)CH(CH₃)CH₂—, —C(O)N(R)CH₂CH(CH₃)CH₂—, —CH₂—, —CH(CH₃)CH₂—, —CH₂C(CH₃)₂CH₂—, —CH₂C(O)—, —CH₂C(O)N(R)C(CH₃)₂CH₂—, —CH₂CH₂C(O)N(R)C(CH₃)₂CH₂—, —CH₂N(R)—, —CH₂N(R)C(O)—, —CH₂N(R)C(O)CH₂CH₂—, —CH₂N(R)C(O)CH(CH₃)CH₂—, —CH₂N(R)C(O)C(CH₃)₂CH₂—, —CH₂N(R)C(O)CH₂C(CH₃)₂CH₂—, —CH₂CH₂N(R)C(O)CH(CH₃)CH₂—, and —CH₂O—. In some embodiments, each L is independently selected from —N(H)C(O)CH₂CH₂—, —N(H)C(O)CH(CH₃)CH₂—, —OCH(CH₂)CH₂—, —C(O)—, —C(O)CH₂CH₂—, —C(O)CH(OH)CH(OH)—, —C(O)N(H)CH(CH₃)CH₂—, —C(O)N(H)CH₂CH(CH₃)CH₂—, —CH₂—, —CH(CH₃)CH₂—, —CH₂C(CH₃)₂CH₂—, —CH₂C(O)—, —CH₂C(O)N(H)C(CH₃)₂CH₂—, —CH₂CH₂C(O)N(H)C(CH₃)₂CH₂—, —CH₂N(H)—, —CH₂N(H)C(O)—, —CH₂N(H)C(O)CH₂CH₂—, —CH₂N(H)C(O)CH(CH₃)CH₂—, —CH₂N(H)C(O)C(CH₃)₂CH₂—, —CH₂N(H)C(O)CH₂C(CH₃)₂CH₂—, —CH₂CH₂N(H)C(O)CH(CH₃)CH₂—, and —CH₂O—.

In some embodiments of any formulae described herein, each L is independently selected from —C(O)CH═CH—, —N(R)C(O)CH═CH—, —C(O)C(≡CH₂)—, —C(O)C(≡CHCH₃)—, —C(O)CH═CH—CH₂—, —C(O)CH═CHCH₂OCH₂—, —C(O)CH═CHCH₂N(R)—, —CH₂N(R)C(O)CH═CH—, —CH₂CH₂N(R)C(O)CH═CH—, —C(O)C≡C—, —C(O)C≡CCH₂—, and —SO₂CH═CH—. In some embodiments, each L is independently selected from —C(O)CH═CH—, —NHC(O)CH═CH—, —C(O)C(≡CH₂)—, —C(O)C(≡CHCH₃)—, —C(O)CH═CH—CH₂—, —C(O)CH═CHCH₂OCH₂—, —C(O)CH═CHCH₂N(CH₃)—, —CH₂NHC(O)CH═CH—, —CH₂CH₂NHC(O)CH═CH—, —C(O)C≡C—, —C(O)C≡CCH₂—, and —SO₂CH═CH—. In some embodiments, each L is independently —C(O)CH═CH—, —C(O)CF=CH—, —NHC(O)CF=CH—, or —NHC(O)CH═CH—. In some embodiments, each L is independently —C(O)CH═CH— or —NHC(O)CH═CH—.

In some embodiments of any formulae described herein, each L is independently selected from —C(O)CClH—, —C(O)CClF—, —C(O)CH═CH—, —N(R)C(O)CH═CH—, —C(O)C(≡CH₂)—, —C(O)C(≡CHCH₃)—, —C(O)CH═CH—CH₂—, —C(O)CH═CHCH₂OCH₂—, —C(O)CH═CHCH₂N(R)—, —CH₂N(R)C(O)CH═CH—, —CH₂CH₂N(R)C(O)CH═CH—, and —SO₂CH═CH—. In some embodiments, each L is independently selected from —C(O)CClF—, —C(O)CH═CH—, —NHC(O)CH═CH—, —C(O)C(≡CH₂)—, —C(O)C(≡CHCH₃)—, —C(O)CH═CH—CH₂—, —C(O)CH═CHCH₂OCH₂—, —C(O)CH═CHCH₂N(CH₃)—, —CH₂NHC(O)CH═CH—, —CH₂CH₂NHC(O)CH═CH—, —C(O)C≡C—, —C(O)C≡CCH₂—, and —SO₂CH═CH—. In some embodiments, each L is independently —C(O)CH═CH— or —NHC(O)CH═CH—.

In some embodiments of any formulae described herein, each W is independently hydrogen, halogen, —CN, or 3-10 membered monocyclic or bicyclic, saturated, partially unsaturated, or aryl ring having 0-3 heteroatoms independently selected from nitrogen, oxygen, or sulfur. In some embodiments, each W is independently hydrogen, halogen, or an optionally substituted 3-10 membered monocyclic or bicyclic, saturated, partially unsaturated, or aryl ring having 0-3 heteroatoms independently selected from nitrogen, oxygen, or sulfur. In some embodiments, each W is independently hydrogen, halogen, or —CN. In some embodiments, W is hydrogen. In some embodiments, W is halogen (e.g., fluoro or chloro). In some embodiments, W is fluoro. In some embodiments, W is chloro. In some embodiments, W is —CN. In some embodiments, W is an optionally substituted 3-10 membered monocyclic or bicyclic, saturated, partially unsaturated, or aryl ring having 0-3 heteroatoms independently selected from nitrogen, oxygen, or sulfur. In some embodiments, W is an optionally substituted group selected from phenyl, a 5- to 6-membered heteroaryl ring having 1-3 heteroatoms independently selected from nitrogen, oxygen, and sulfur, a 3- to 6-membered carbocyclic ring, and a 4- to 6-membered heterocyclic ring having 1-2 heteroatoms independently selected from nitrogen, oxygen, and sulfur. In some embodiments, W is an optionally substituted 3- to 6-membered carbocyclic ring. In some embodiments, W is a cyclopentane. In some embodiments, W is an optionally substituted 4- to 6-membered heterocyclic ring having 1-2 heteroatoms independently selected from nitrogen, oxygen, and sulfur. In some embodiments, W is a pyrrolidine.

In some embodiments of any formulae described herein, each X is independently halogen or —CN. In some embodiment, each X is independently halogen (e.g., chloro or fluoro). In some embodiments, each X is independently chloro. In some embodiments, each X is independently fluoro. In some embodiments, each X is independently —CN. In some embodiments, each X is independently —OR (e.g., —OH).

In some embodiments of any formulae described herein, each -L-W is independently selected from —CH₃, —CH(CH₃)₂, —CH₂C(CH₃)₃, —CH₂OH, —CH₂NH₂, —OCH(CH₃)₂, —CN,

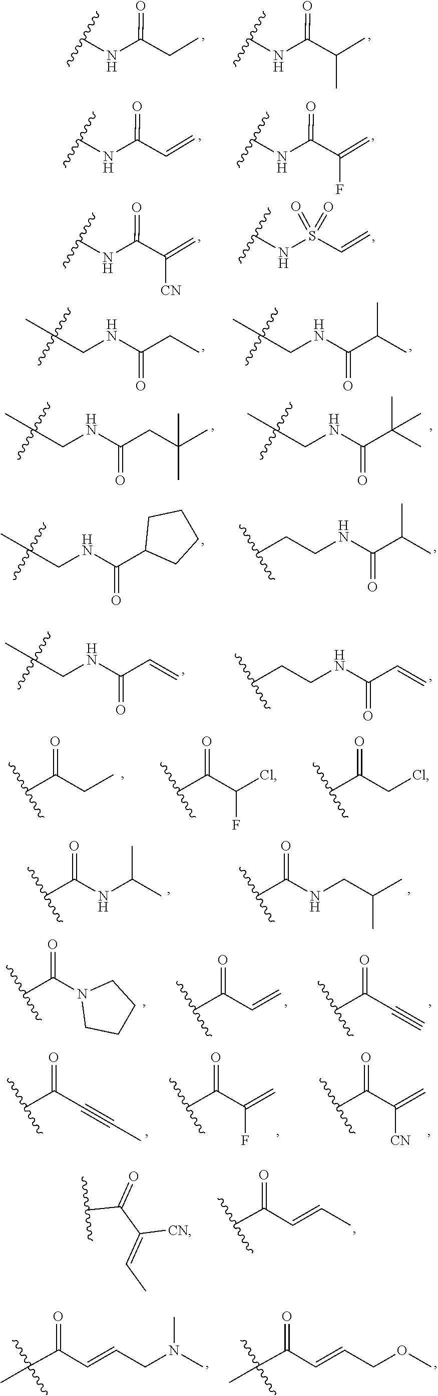

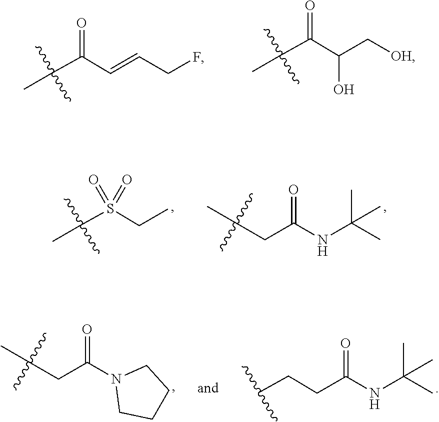

In some embodiments of any formulae described herein, each -L-W is independently selected from —CH₃, —CH(CH₃)₂, —CH₂C(CH₃)₃, —CH₂OH, —CH₂NH₂, —OCH(CH₃)₂, —CN, —C(O)CH═CH₂, —C(O)CF=CH₂, —NHC(O)CF=CH₂, and —NHC(O)CH═CH₂.

In some embodiments of any formulae described herein, each -L-W is independently selected from —C(O)CH═CH₂ and —NHC(O)CH═CH₂. In some embodiments, -L-W is independently selected from —C(O)CH═CH₂, —C(O)CF=CH₂, —NHC(O)CF=CH₂, and —NHC(O)CH═CH₂. In some embodiments, each -L-W is —C(O)CH═CH₂. In some embodiments, each -L-W is —CN.

In some embodiments of any formulae described herein, each Ring D′ is independently a 4- to 6-membered saturated or partially unsaturated carbocyclic ring or a 4- to 6-membered saturated or partially unsaturated heterocyclic ring having 1-2 heteroatoms independently selected from nitrogen, oxygen, and sulfur, wherein Ring D′ is substituted with t instances of -L-W.

In some embodiments of any formulae described herein, Ring D′ is a 4- to 6-membered carbocyclic ring substituted with t instances of -L-W. In some embodiments, Ring D′ is a 4- to 6-membered saturated or partially unsaturated carbocyclic ring substituted with t instances of -L-W. In some embodiments, Ring D′ is a 4- to 6-membered cycloalkyl ring substituted with t instances of -L-W. In some embodiments, Ring D′ is a 4-membered carbocyclic ring substituted with t instances of -L-W. In some embodiments, Ring D′ is a cyclobutane substituted with t instances of -L-W. In some embodiments, Ring D′ is a 5-membered carbocyclic ring substituted with t instances of -L-W. In some embodiments, Ring D′ is a 6-membered carbocyclic ring substituted with t instances of -L-W.

In some embodiments of any formulae described herein, Ring D′ is a 4- to 6-membered heterocyclic ring having 1-2 heteroatoms independently selected from nitrogen, oxygen, and sulfur, wherein Ring D′ is substituted with t instances of -L-W. In some embodiments, Ring D′ is a 4- to 6-membered saturated or partially unsaturated heterocyclic ring having 1-2 heteroatoms independently selected from nitrogen, oxygen, and sulfur, wherein Ring D′ is substituted with t instances of -L-W. In some embodiments, Ring D′ is a 4- to 6-membered saturated heterocyclic ring having 1-2 heteroatoms independently selected from nitrogen, oxygen, and sulfur, wherein Ring D′ is substituted with t instances of -L-W. In some embodiments, Ring D′ is a 4-membered heterocyclic ring having 1-2 heteroatoms independently selected from nitrogen, oxygen, and sulfur, wherein Ring D′ is substituted with t instances of -L-W. In some embodiments, Ring D′ is an azetidine substituted with t instances of -L-W. In some embodiments, Ring D′ is a 5-membered heterocyclic ring having 1-2 heteroatoms independently selected from nitrogen, oxygen, and sulfur, wherein Ring D′ is substituted with t instances of -L-W. In some embodiments, Ring D′ is a pyrrolidine substituted with t instances of -L-W. In some embodiments, Ring D′ is a 6-membered heterocyclic ring having 1-2 heteroatoms independently selected from nitrogen, oxygen, and sulfur, wherein Ring D′ is substituted with t instances of -L-W. In some embodiments, Ring D′ is a piperidine substituted with t instances of -L-W.

In some embodiments of any formulae described herein, Ring D′, substituted with t instances of -L-W, is selected from

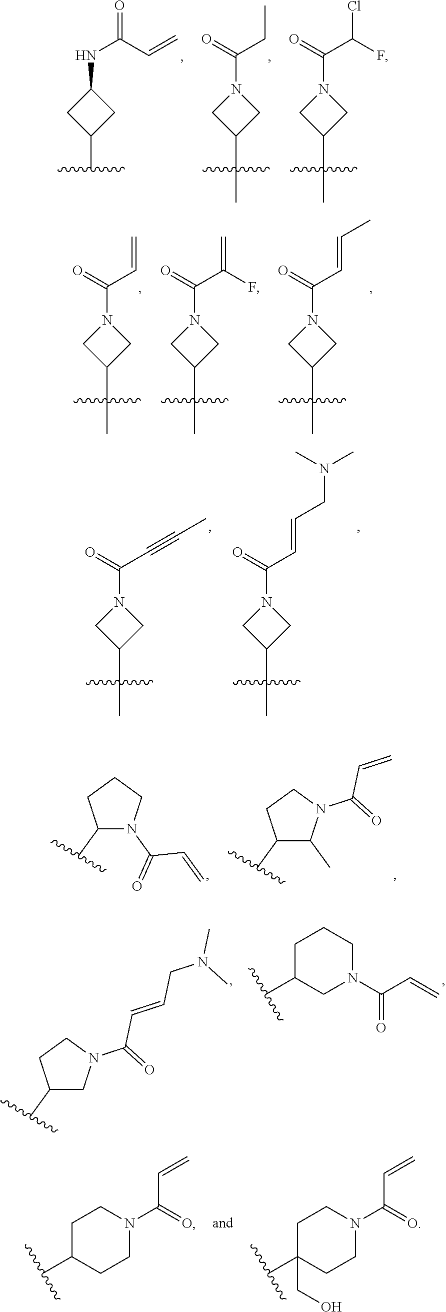

In some embodiments of any formulae described herein, when R¹ is Ring D′ or a bivalent C₁₋₆ aliphatic chain substituted with Ring D′, then Ring D, substituted with r instances of R², is selected from

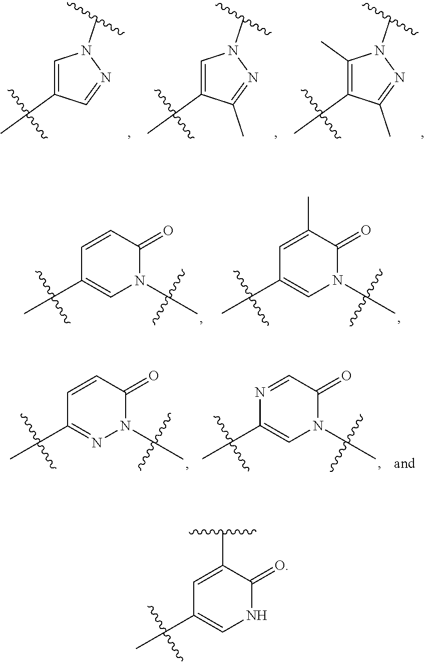

In some embodiments of any formulae described herein, Ring D is substituted with 1 instance of R¹, R¹ is Ring D′, and

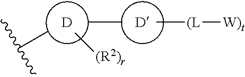

is selected from

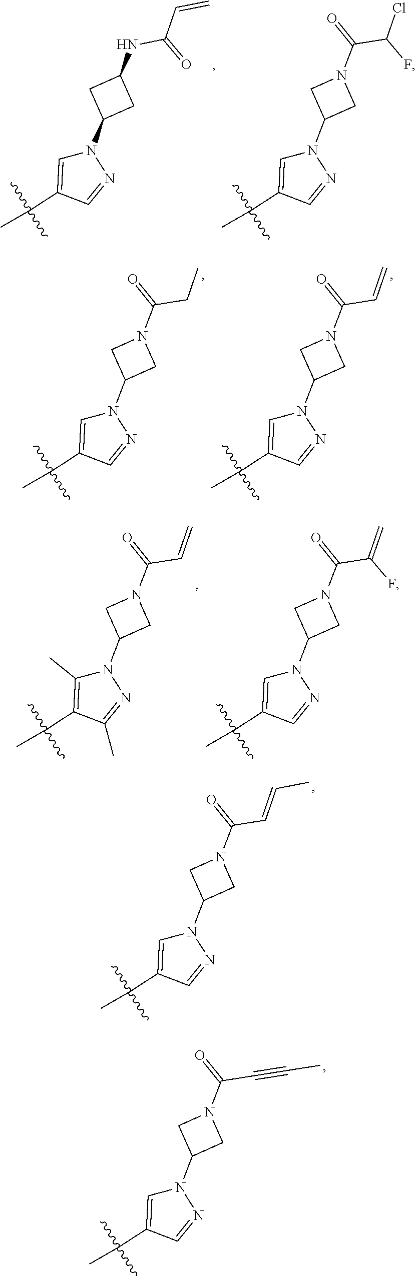

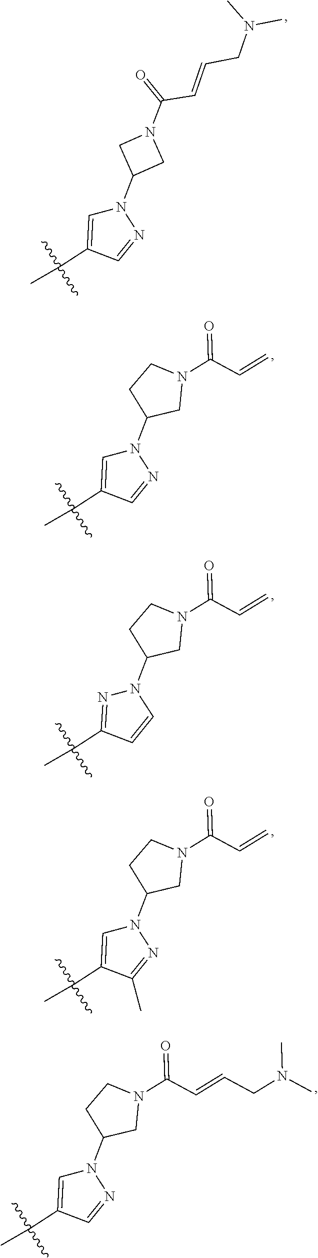

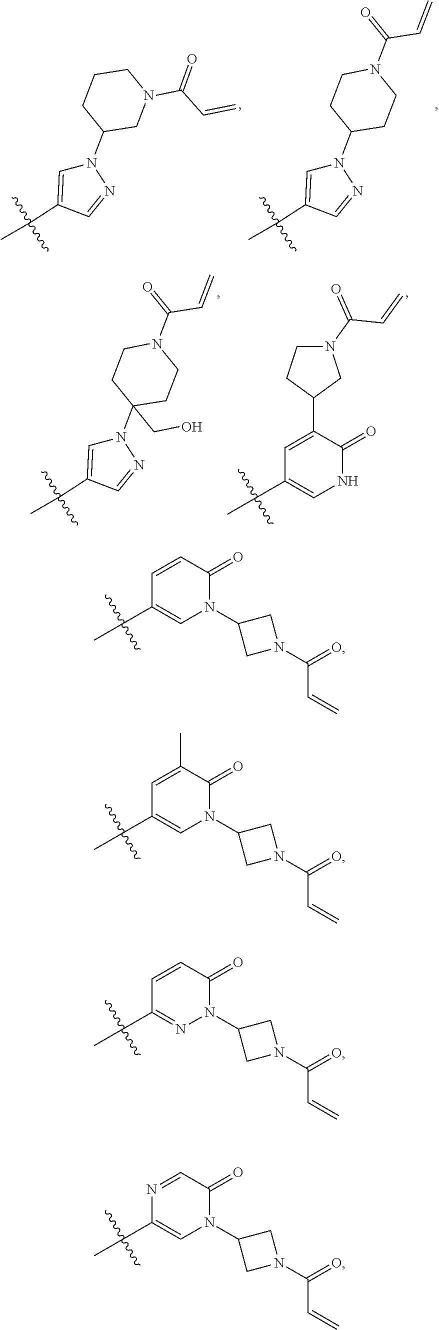

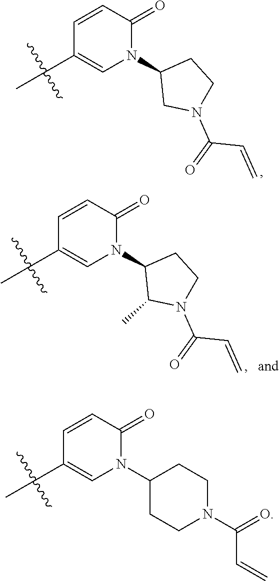 [SMILES: *c1ccc(=O)n([C@H]2CCN(C(=O)C=C)C2)c1 ; *c1ccc(=O)n([C@H]2CCN(C(=O)C=C)[C@@H]2C)c1 ; *c1ccc(=O)n(C2CCN(C(=O)C=C)CC2)c1]

In some embodiments of any formulae described herein, q is 0. In some embodiments, q is 1.

In some embodiments of any formulae described herein, t is 0 or 1. In some embodiments, t is 1 or 2. In some embodiments, t is 0. In some embodiments, t is 1. In some embodiments, t is 2.

In some embodiments of any formulae described herein, each R² is independently selected from halogen, —CN, —OR, and C₁₋₆ alkyl. In some embodiments, each R² is independently selected from oxo, halogen, —CN, —O(C₁₋₄ alkyl), and C₁₋₆ alkyl. In some embodiments, R² is oxo. In some embodiments, R² is halogen (e.g., fluoro or chloro). In some embodiments, R² is —CN. In some embodiments, R² is —OR. In some embodiments, R² is —O(C₁₋₄ alkyl). In some embodiments, R² is C₁₋₆ alkyl. In some embodiments, each R² is independently selected from oxo, fluoro, chloro, —CN, —OCH₃, —OCH(CH₃)₂, —CH₃, —CH(CH₃)₂, and —CH₂C(CH₃)₃.

In some embodiments of any formulae described herein, r is 0 or 1. In some embodiments, r is 1 or 2. In some embodiments, when q is 1, r is 0 or 1. In some embodiments, when q is 0, r is 1 or 2. In some embodiments, r is 0. In some embodiments, r is 1. In some embodiments, r is 2.

In some embodiments of any formulae described herein, each R^{2′} is independently selected from halogen, —CN, —OR, and C₁₋₆ alkyl. In some embodiments, R^{2′} is oxo. In some embodiments, R^{2′} is halogen (e.g., fluoro or chloro). In some embodiments, R^{2′} is —CN. In some embodiments, R^{2′} is —OR. In some embodiments, R² is C₁₋₆ alkyl (e.g., methyl).

In some embodiments of any formulae described herein, y is 0 or 1. In some embodiments, y is 1 or 2. In some embodiments, y is 0. In some embodiments, y is 1. In some embodiments, y is 2.

In some embodiments of any formulae described herein, each Cy is independently a 5- to 6-membered heteroaryl ring having 1-3 heteroatoms independently selected from nitrogen, oxygen, and sulfur, a 3- to 6-membered saturated or partially unsaturated carbocyclic ring, or a 4- to 6-membered saturated or partially unsaturated heterocyclic ring having 1-2 heteroatoms independently selected from nitrogen, oxygen, and sulfur, wherein Cy is substituted with 0-2 instances of R. In some embodiments, Cy is substituted with 0-1 instances of R. In some embodiments, Cy is substituted with 1-2 instances of R. In some embodiments, Cy is not substituted with R⁶. In some embodiments, Cy is substituted with 1 instance of R⁶. In some embodiments, Cy is substituted with 2 instances of R.

In some embodiments of any formulae described herein, Cy is a 5- to 6-membered heteroaryl ring having 1-3 heteroatoms independently selected from nitrogen, oxygen, and sulfur, wherein Cy is substituted with 0-2 instances of R⁶. In some embodiments, Cy is a 5-membered heteroaryl ring having 1-3 heteroatoms independently selected from nitrogen, oxygen, and sulfur, wherein Cy is substituted with 0-2 instances of R⁶. In some embodiments, Cy is a 6-membered heteroaryl ring having 1-3 nitrogen atoms, wherein Cy is substituted with 0-2 instances of R⁶. In some embodiments, Cy is a pyridine substituted with 0-2 instances of R.

In some embodiments of any formulae described herein, Cy is a 3- to 6-membered carbocyclic ring substituted with 0-2 instances of R⁶. In some embodiments, Cy is a 3- to 6-membered saturated or partially unsaturated carbocyclic ring substituted with 0-2 instances of R⁶. In some embodiments, Cy is a 3- to 6-membered cycloalkyl ring substituted with 0-2 instances of R⁶. In some embodiments, Cy is a 3-membered carbocyclic ring (e.g., a cyclopropyl ring) substituted with 0-2 instances of R⁶. In some embodiments, Cy is a 4-membered carbocyclic ring substituted with 0-2 instances of R⁶. In some embodiments, Cy is a 5-membered carbocyclic ring substituted with 0-2 instances of R⁶. In some embodiments, Cy is a 6-membered carbocyclic ring substituted with 0-2 instances of R.

In some embodiments of any formulae described herein, Cy is a 4- to 6-membered heterocyclic ring having 1-2 heteroatoms independently selected from nitrogen, oxygen, and sulfur. In some embodiments, Cy is a 4- to 6-membered saturated or partially unsaturated heterocyclic ring having 1-2 heteroatoms independently selected from nitrogen, oxygen, and sulfur. In some embodiments, Cy is a 4-membered heterocyclic ring having 1-2 heteroatoms independently selected from nitrogen, oxygen, and sulfur. In some embodiments, Cy is an azetidine. In some embodiments, Cy is a 5-membered heterocyclic ring having 1-2 heteroatoms independently selected from nitrogen, oxygen, and sulfur. In some embodiments, Cy is a pyrrolidone. In some embodiments, Cy is a 6-membered heterocyclic ring having 1-2 nitrogen atoms. In some embodiments, Cy is a piperidinone or a piperazine.

In some embodiments of any formulae described herein, Cy, substituted with 0-2 instances of R⁶, is selected from

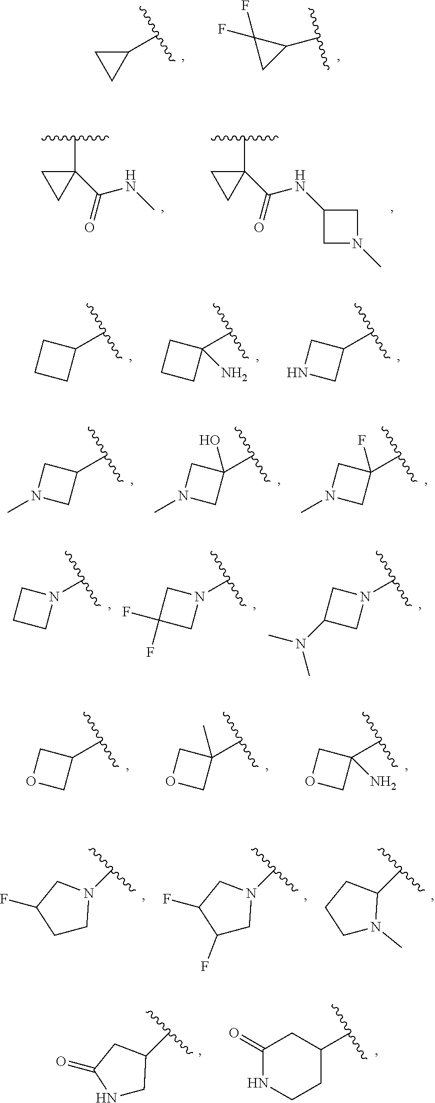

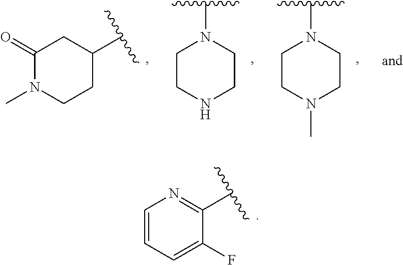

In some embodiments of any formulae described herein, each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and a group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur. In some embodiments, each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and an optionally substituted group selected from C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-3 heteroatoms independently selected from nitrogen, oxygen, and sulfur, a 10-membered aryl ring, and a 9- to 10-membered heteroaryl ring having 1-3 heteroatoms independently selected from nitrogen, oxygen, and sulfur. In some embodiments, each R⁶ is independently selected from —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and an optionally substituted group selected from C₁₋₆ aliphatic, a 3- to 6-membered carbocyclic ring, phenyl, a 3- to 6-membered heterocyclic ring having 1-3 heteroatoms independently selected from nitrogen, oxygen, and sulfur, a 5- to 6-membered heteroaryl ring having 1-3 heteroatoms independently selected from nitrogen, oxygen, and sulfur, a 10-membered aryl ring, and a 9- to 10-membered heteroaryl ring having 1-3 heteroatoms independently selected from nitrogen, oxygen, and sulfur. In some embodiments, each R⁶ is independently selected from oxo, halogen, —OR, —N(R)₂, —C(O)N(R)₂, and optionally substituted C₁₋₆ aliphatic. In some embodiments, each R⁶ is independently selected from oxo, halogen, and optionally substituted C₁₋₆ aliphatic. In some embodiments, each R⁶ is independently selected from oxo, halogen, and optionally substituted C₁₋₆ alkyl. In some embodiments, each R⁶ is independently selected from halogen and optionally substituted C₁₋₆ aliphatic. In some embodiments, each R⁶ is independently selected from halogen and optionally substituted C₁₋₆ alkyl. In some embodiments, R⁶ is oxo. In some embodiments, R⁶ is halogen (e.g., fluoro or chloro). In some embodiments, R⁶ is fluoro. In some embodiments, R⁶ is —OR (e.g., —OH). In some embodiments, R⁶ is —N(R)₂ (e.g., —N(C₁₋₄ alkyl)₂ or —NH₂). In some embodiments, R⁶ is —C(O)N(R)₂. In some embodiments, R⁶ is optionally substituted C₁₋₆ aliphatic. In some embodiments, R⁶ is optionally substituted C₁₋₆ alkyl. In some embodiments, R⁶ is C₁₋₆ alkyl (e.g., methyl).

In some embodiments of any formulae described herein, each v is independently 0 or 1. In some embodiments, each v is independently 1 or 2. In some embodiments, v is 0. In some embodiments, v is 1. In some embodiments, v is 2.

In some embodiments of any formulae described herein, each x is independently 0 or 1. In some embodiments, each x is independently 1 or 2. In some embodiments, x is 0. In some embodiments, x is 1. In some embodiments, x is 2.

In some embodiments of any formulae described herein, each R is independently hydrogen, C₁₋₆ aliphatic, C₁₋₆ haloaliphatic, a 3- to 6-membered carbocyclic ring, phenyl, a 3- to 6-membered heterocyclic ring having 1-3 heteroatoms independently selected from nitrogen, oxygen, and sulfur, a 5- to 6-membered heteroaryl ring having 1-3 heteroatoms independently selected from nitrogen, oxygen, and sulfur, a 10-membered aryl ring, and a 9- to 10-membered heteroaryl ring having 1-3 heteroatoms independently selected from nitrogen, oxygen, and sulfur. In some embodiments, each R is independently hydrogen or an optionally substituted group selected from C₁₋₆ aliphatic, a 3- to 6-membered saturated or partially unsaturated carbocyclic ring, phenyl, a 3- to 6-membered saturated or partially unsaturated heterocyclic ring having 1-3 heteroatoms independently selected from nitrogen, oxygen, and sulfur, a 5- to 6-membered heteroaryl ring having 1-3 heteroatoms independently selected from nitrogen, oxygen, and sulfur, a 10-membered aryl ring, and a 9- to 10-membered heteroaryl ring having 1-3 heteroatoms independently selected from nitrogen, oxygen, and sulfur. In some embodiments, each R is independently hydrogen, optionally substituted C₁₋₆ aliphatic, or optionally substituted 4- to 6-membered heterocyclic ring having 1-2 heteroatoms independently selected from nitrogen, oxygen, and sulfur. In some embodiments, each R is independently hydrogen, C₁₋₆ alkyl optionally substituted with one or more halogen (e.g., fluoro), or a 4- to 6-membered heterocyclic ring having 1-2 heteroatoms independently selected from nitrogen, oxygen, and sulfur and optionally substituted with C₁₋₆ alkyl. In some embodiments, each R is independently hydrogen or optionally substituted C₁₋₆ aliphatic. In some embodiments, each R is independently hydrogen or optionally substituted C₁₋₆ alkyl. In some embodiments, each R is independently hydrogen or C₁₋₆ alkyl optionally substituted with one or more halogen (e.g., fluoro). In some embodiments, R is hydrogen. In some embodiments, R is optionally substituted C₁₋₆ aliphatic. In some embodiments, R is C₁₋₆ haloaliphatic. In some embodiments, R is optionally substituted C₁₋₆ alkyl. In some embodiments, R is C₁₋₆ alkyl optionally substituted with one or more halogen (e.g., fluoro).

Also provided herein are embodiments wherein any embodiment described herein may be combined with any one or more of these embodiments, provided the combination is not mutually exclusive. As used herein, two embodiments are “mutually exclusive” when one is defined to be something which is different than the other. For example, an embodiment wherein two groups combined to form a ring is mutually exclusive with an embodiment in which one group is ethyl and the other group is hydrogen. Similarly, an embodiment wherein one group is CH₂ is mutually exclusive with an embodiment wherein the same group is NH.

In some embodiments of any of the preceding aspects, the compound is a compound included in Table 1 or 2 or a salt (e.g., pharmaceutically acceptable salt), ester, tautomer, prodrug, zwitterionic form, or stereoisomer thereof. In some embodiments, the compound is a compound included in Table 1, or a salt (e.g., pharmaceutically acceptable salt), ester, tautomer, prodrug, zwitterionic form, or stereoisomer thereof. In some embodiments, the compound is a compound included in Table 1, or a salt (e.g., pharmaceutically acceptable salt) thereof. In some embodiments, the compound is a compound included in Table 2, or a salt (e.g., pharmaceutically acceptable salt), ester, tautomer, prodrug, zwitterionic form, or stereoisomer thereof. In some embodiments, the compound is a compound included in Table 2, or a salt (e.g., pharmaceutically acceptable salt) thereof.

Also provided herein is a compound selected from Table 1 or 2 or any of the Examples provided herein, or a salt (e.g., pharmaceutically acceptable salt), ester, tautomer, prodrug, zwitterionic form, or stereoisomer thereof. In some embodiments, the present disclosure provides a compound selected from Table 1 or 2 or any of the Examples provided herein, or a salt (e.g., pharmaceutically acceptable salt) thereof.

A compound of the present disclosure, such as a compound of a formula included in Table 1 or 2, may be synthesized according to one of the general routes outlined in Synthetic Examples 1-88 or by various other methods generally known in the art.

Table 1 includes selected compounds of the present disclosure.

TABLE 1
Selected compounds of the present disclosure.

| Compound | | |
|---|---|---|
| No. | Structure | Analytical data |
| 1 | 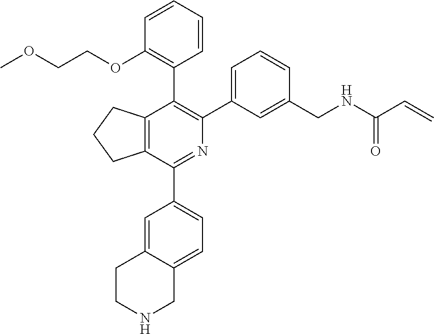 [SMILES: C=CC(=O)NCc1cccc(-c2nc(-c3ccc4c(c3)CCNC4)c3c(c2-c2ccccc2OCCOC)CCC3)c1] | LCMS m/z [M + 1]: 560.2 ¹H NMR (400 MHz, CD₃OD) δ:7.74-7.76 (m, 2H), 7.48-7.50 (d, J = 8.4 Hz, 1H), 7.40 (s, 1H), 7.26-7.35 (m, 4H), 7.02-7.04 (d, J = 8.4 Hz, 1H), 6.96-6.98 (d, J = 7.6 Hz, 1H), 6.86-6.90 (t, J = 7.2 Hz, 1H), 6.24-6.25 (d, J = 7.2 Hz, 2H), 5.69-5.72 (t, J = 5.2 Hz, 1H), 4.50 (s,2H), 4.33-4.61 (m, 2H), 4.12-4.15 (m, 1H), 3.95- 3.98 (m, 1H), 3.57-3.61 (m, 4H), 3.06-3.28 (m, 8H), 2.85-2.93 (m, 1H), 2.18-2.24 (m, 2H). |
| 2 | 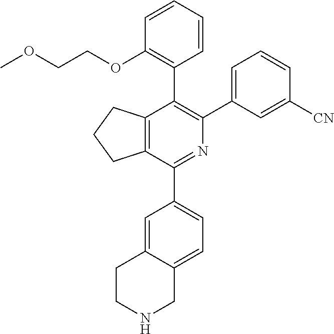 [SMILES: COCCOc1ccccc1-c1c(-c2cccc(C#N)c2)nc(-c2ccc3c(c2)CCNC3)c2c1CCC2] | LCMS m/z [M + 1]: 502.3 ¹H NMR (400 MHz, CD₃OD) δ: 7.65-7.71 (m, 3H), 7.50 (dd, J = 9.3, 2.5 Hz, 1H), 7.31-7.36 (m, 2H), 7.06-7.08 (m, 2H), 6.99 (t, J = 7.2 Hz, 1H), 6.32 (d, J = 9.3 Hz, 1H), 5.48 (s, 1H), 4.35 (s, 2H), 4.08-4.12 (m, 1H), 4.01-4.05 (m, 1H), 3.58 (t, J = 4.5 Hz, 2H), 3.46 (t, J = 6.2 Hz, 2H), 3.44 (s, 3H), 3.24 (s, 3H), 3.14-3.18 (m, 4H), 2.70-2.84 (m, 2H), 2.06-2.10 (m, 2H). |
| 3 | 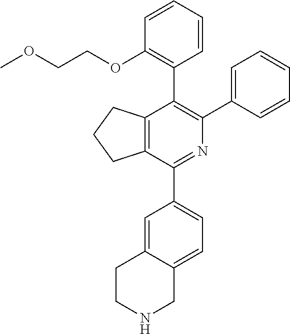 [SMILES: COCCOc1ccccc1-c1c(-c2ccccc2)nc(-c2ccc3c(c2)CCNC3)c2c1CCC2] | LCMS m/z [M + 1]: 477.3 ¹H NMR (400 MHz, CD₃OD) δ: 7.74-7.75 (m, 2H), 7.50 (d, J = 8.5 Hz, 1H), 7.39-7.45 (m, 3H), 7.32-7.36 (m, 3H), 7.05 (d, J = 8.4 Hz, 1H), 6.98 (dd, J = 7.5, 1.5 Hz, 1H), 6.89 (t, J = 7.4 Hz, 1H), 4.50 (s, 2H), 4.12- 4.16 (m, 1H), 3.98-4.02 (m, 1H), 3.57-3.63 (m, 4H), 3.31 (s, 3H), 3.09-3.27 (m, 5H), 2.87-2.95 (m, 1H), 2.20-2.25 (m, 2H). |
| 4 | 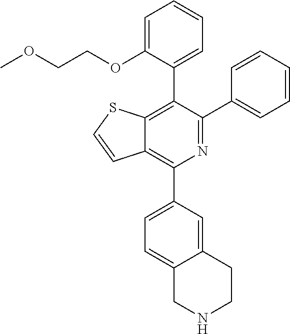 [SMILES: COCCOc1ccccc1-c1c(-c2ccccc2)nc(-c2ccc3c(c2)CCNC3)c2ccsc12] | LCMS m/z [M + 1]: 493.2 ¹H NMR (400 MHz, CD₃OD) δ: 7.95 (d, J = 4.8 Hz, 1H), 7.85-7.88 (m, 2H), 7.72 (d, J = 5.6 Hz, 1H), 7.47- 7.55 (m, 3H), 7.42 (t, J = 8.0 Hz, 1H), 7.24-7.38 (m, 4H), 7.08 (d, J = 8.4 Hz, 1H), 7.02 (t, J = 7.4 Hz, 1H), 4.52 (s, 2H), 4.00-4.08 (m, 1H), 3.84-3.88 (m, 1H), 3.60 (t, J = 6.2 Hz, 2H), 3.42-3.53 (m, 2H), 3.15 (s, 3H). |
| 5 | 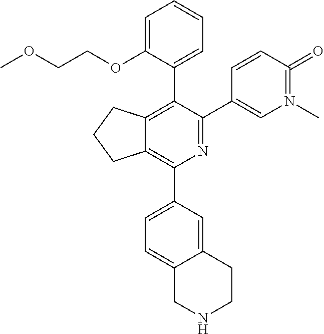 | LCMS m/z [M + 1]: 508.3 ¹H NMR (400 MHz, CD₃OD) δ: 7.65-7.71 (m, 3H), 7.50 (dd, J = 9.3, 2.5 Hz, 1H), 7.31-7.36 (m, 2H), 7.06- 7.08 (m, 2H), 6.99 (t, J = 7.2 Hz, 1H), 6.32 (d, J = 9.3 Hz, 1H), 5.48 (s, 1H), 4.35 (s, 2H), 4.08-4.12 (m, 1H), 4.01-4.05 (m, 1H), 3.58 (t, J = 4.5 Hz, 2H), 3.46 (t, J = 6.2 Hz, 2H), 3.44 (s, 3H), 3.24 (s, 3H), 3.14- 3.18 (m, 4H), 2.70-2.84 (m, 2H), 2.06-2.10 (m, 2H). |
| 6 | 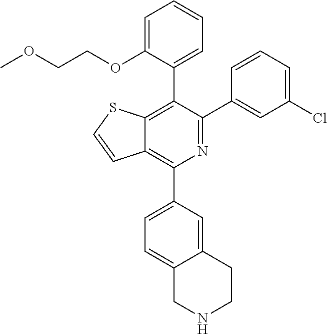 [SMILES: COCCOc1ccccc1-c1c(-c2cccc(Cl)c2)nc(-c2ccc3c(c2)CCNC3)c2ccsc12] | LCMS m/z [M + 1]: 527.0 ¹H NMR (400 MHz, CD₃OD) δ 7.84-7.86 (m, 3H), 7.68(d, J = 5.6 Hz, 1H), 7.57(s, 1H), 7.48 (d, J = 8.0 Hz, 1H), 7.43 (t, J = 7.2 Hz, 1H), 7.36 (d, J = 8.0 Hz, 1H), 7.29 (t, J = 5.6 Hz, 2H), 7.20-7.22 (m, 1H), 7.03- 7.10 (m, 2H), 4.50 (s, 2H), 4.01-4.05 (m, 1H), 3.82- 3.88 (m, 1H), 3.60 (t, J = 6.0 Hz, 2H), 3.45-3.47 (m, 2H), 3.14 (s, 3H). |
| 7 | 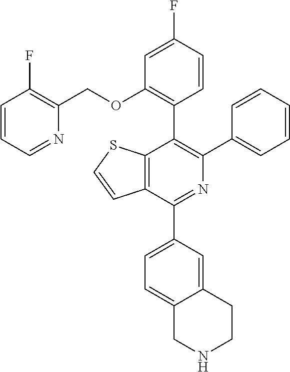 [SMILES: Fc1ccc(-c2c(-c3ccccc3)nc(-c3ccc4c(c3)CCNC4)c3ccsc23)c(OCc2ncccc2F)c1] | LCMS m/z [M + 1]: 662.2 ¹H NMR (400 MHz, CD₃OD) δ: 8.31 (d, J = 4.7 Hz, 1H), 7.90-7.96 (m, 1H), 7.80-7.85 (m, 2H), 7.66-7.72 (m, 1H), 7.56 (t, J = 9.0 Hz, 1H), 7.50 (d, J = 7.8 Hz, 1H), 7.39-7.45 (m, 3H), 7.23-7.38 (m, 4H), 7.12-7.18 (m, 1H), 6.75-6.82 (m, 1H), 5.09-5.16 (m, 2H), 4.51 (s, 2H), 3.59 (t, J = 6.3 Hz, 2H), 3.25-3.29 (m, 2H). |
| 8 | 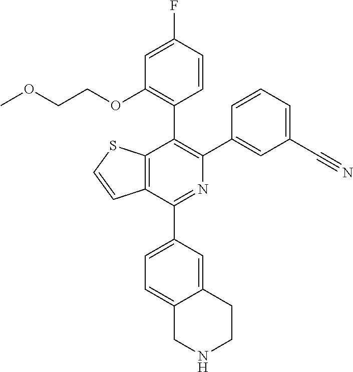 [SMILES: COCCOc1cc(F)ccc1-c1c(-c2cccc(C#N)c2)nc(-c2ccc3c(c2)CCNC3)c2ccsc12] | LCMS m/z [M + 1]: 536 ¹H NMR (400 MHz, CD₃OD) δ 7.89 (s, 1H), 7.81- 7.86 (m, 3H), 7.68-7.72 (m, 2H), 7.63 (d, J = 8 Hz, 1H), 7.39-7.47 (m, 2H), 7.32-7.35 (m, 1H), 6.90 (dd, J = 11.1, 2.2 Hz, 1H), 6.79-6.83 (m, 1H), 4.49 (s, 2H), 3.98-4.01 (m, 1H), 3.83-3.86 (m, 1H), 3.60 (t, J = 6.4 Hz, 2H), 3.44 (t, J = 4.8, 2H), 3.26-3.30 (m, 2H), 3.15 (s, 3H). |
| 9 | 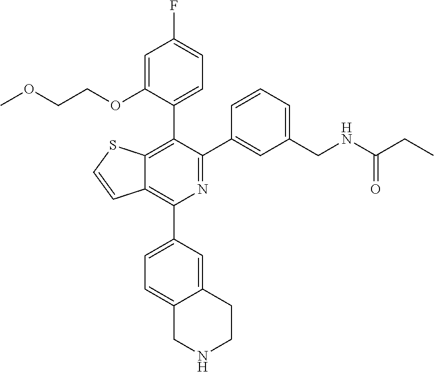 [SMILES: CCC(=O)NCc1cccc(-c2nc(-c3ccc4c(c3)CCNC4)c3ccsc3c2-c2ccc(F)cc2OCCOC)c1] | LCMS m/z [M + 1]: 596 ¹H NMR (400 MHz, CD₃OD) δ 7.83-7.84 (m, 3H), 7.67 (d, J = 5.6, 1H), 7.46-7.48 (m, 2H), 7.34-7.35 (m, 1H), 7.23-7.27 (m, 3H), 6.89 (d, J = 11.2, 2.3 Hz, 1H), 6.73-6.80 (m, 1H), 4.50 (s, 2H), 4.28-4.32 (m, 2H), 4.0-4.08 (m, 1H), 3.80-3.88 (m, 1H), 3.59 (t, J = 6.4 Hz, 2H), 3.41-3.47 (m, 2H), 3.20-3.30(m, 2H), 3.13 (s, 3H), 2.22 (q, J = 7.6 Hz, 2H), 1.13 (t, J = 7.6 Hz, 3H). |
| 10 | 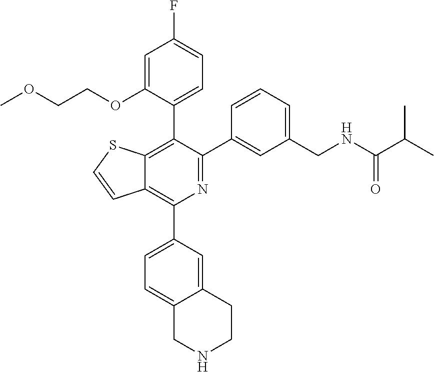 [SMILES: COCCOc1cc(F)ccc1-c1c(-c2cccc(CNC(=O)C(C)C)c2)nc(-c2ccc3c(c2)CCNC3)c2ccsc12] | LCMS m/z [M + 1]: 610 ¹H NMR (400 MHz, CD₃OD) δ7.92 (d, J = 5.6 Hz, 1H), 7.84-7.86 (m, 2H), 7.70 (d, J = 5.6 Hz, 1H), 7.48-7.51 (m, 2H), 7.32-7.33 (m, 1H), 7.24-7.28 (m, 3H), 6.91 (dd, J = 11.2, 2.4 Hz, 1H), 6.75-6.76 (m, 1H), 4.51 (s, 2H), 4.31 (d, J = 5.6 Hz, 2H), 4.02-4.10 (m, 1H), 3.83-3.87 (m, 1H), 3.60 (t, J = 6.4 Hz, 2H), 3.46-3.48 (m, 2H), 3.27-3.30 (m, 2H), 3.14 (s, 3H), 2.44-2.48 (m, 1H), 1.12 (d, J = 6.8 Hz, 6H). |
| 11 | 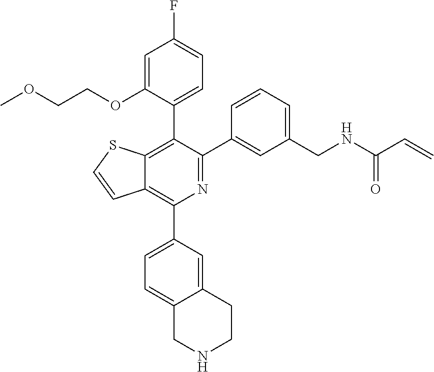 [SMILES: C=CC(=O)NCc1cccc(-c2nc(-c3ccc4c(c3)CCNC4)c3ccsc3c2-c2ccc(F)cc2OCCOC)c1] | LCMS m/z [M + 1]: 594.3 ¹H NMR (400 MHz, CD₃OD) δ 7.91 (d, J = 5.6 Hz, 1H), 7.84-7.87 (m, 2H), 7.70 (d, J = 5.6 Hz, 1H), 7.45-7.51 (m, 2H), 7.36-7.40 (m, 1H), 7.22-7.32 (m, 3H), 6.88 (dd, J = 11.1, 2.2 Hz, 1H), 6.75 (td, J = 8.3, 2.3 Hz, 1H), 6.25 (d, J = 6.0 Hz, 2H), 5.70 (t, J = 6.0 Hz, 1H), 4.51 (s, 2H), 4.40 (s, 2H), 3.96-4.08 (m, 1H), 3.78-3.91 (m, 1H), 3.60 (t, J = 6.3 Hz, 1H), 3.44-3.47 (m, 2H), 3.13 (s, 3H). |
| 12 | 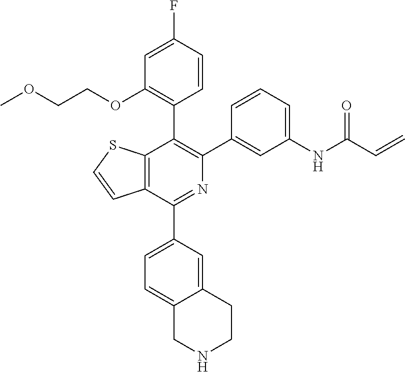 [SMILES: C=CC(=O)Nc1cccc(-c2nc(-c3ccc4c(c3)CCNC4)c3ccsc3c2-c2ccc(F)cc2OCCOC)c1] | LCMS m/z [M + 1]: 580.2 ¹H NMR (400 MHz, CD₃OD) δ 8.06 (s, 1H), 7.83- 7.87 (m, 3H), 7.68 (d, J = 5.6 Hz, 1H), 7.45-7.49 (m, 2H), 7.29 (dd, J = 8.4, 6.8 Hz, 1H), 7.20 (t, J = 7.8 Hz, 1H), 7.10-7.14 (m, 1H), 6.88 (dd, J = 11.0, 1.0 Hz, 1H), 6.74-6.80 (m, 1H), 6.32-6.46 (m, 2H), 5.76 (dd, J = 9.6, 2.1 Hz, 1H), 4.50 (s, 2H), 3.99-4.50 (m, 1H), 3.82-3.87 (m, 1H), 3.60 (t, J = 6.4 Hz, 2H), 3.45- 3.49 (m, 2H), 3.12 (s, 2H). |
| 13 | 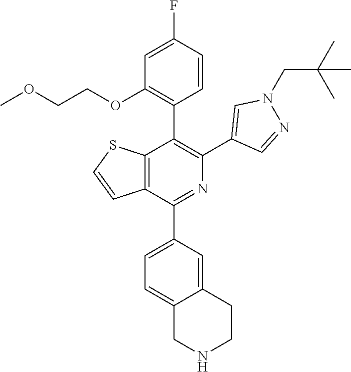 [SMILES: COCCOc1cc(F)ccc1-c1c(-c2cnn(CC(C)(C)C)c2)nc(-c2ccc3c(c2)CCNC3)c2ccsc12] | LCMS m/z [M + 1]: 571.3 ¹H NMR (400 MHz, CD₃OD) δ 7.80-7.86 (m, 2H), 7.75 (d, J = 5.6 Hz, 1H), 7.63 (d, J = 5.6 Hz, 1H), 7.59 (s, 1H), 7.47(d, J = 7.7 Hz, 1H), 7.30-7.36 (m, 2H), 7.04-7.07 (m, 1H), 6.89-6.94 (m, 1H), 4.51 (s, 2 H), 3.97-4.09 (m, 2H), 3.84 (s, 2H), 3.60 (t, J = 6.4 Hz, 2H), 3.43 (t, J = 4.4 Hz, 2H), 3.06 (s, 3H), 0.86 (s, 9H). |
| 14 | 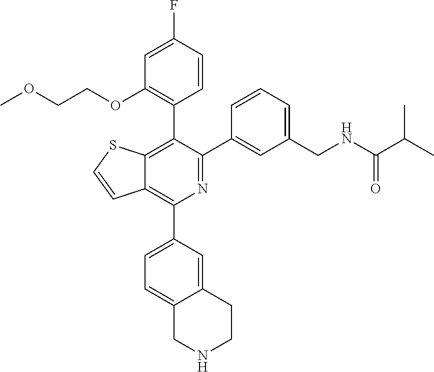 [SMILES: COCCOc1cc(F)ccc1-c1c(-c2cccc(CNC(=O)C(C)C)c2)nc(-c2ccc3c(c2)CCNC3)c2ccsc12] | LCMS m/z [M + 1]: 610 ¹H NMR (400 MHz, CD₃OD) δ 8.03 (s, 1H), 7.94 (d, J = 8 Hz, 1H), 7.85-7.88 (m, 2H), 7.78 (d, J = 7.6 Hz, 1H), 7.73 (d, J = 5.6 Hz, 1H), 7.60 (d, J = 8 Hz, 1H), 7.51 (d, J = 8.4 Hz, 1H), 7.39 (t, J = 6.4 Hz, 1H), 7.32 (t, J = 1.6 Hz, 1H), 6.87-6.90 (m, 1H), 6.75-6.80 (m, 1H), 4.52 (s, 2H), 4.00-4.05 (m, 1H), 3.79-3.84 (m, 1H), 3.60 (t, J = 6.4 Hz, 2H), 3.41-3.52 (m, 2H), 3.27- 3.31 (m, 2H), 3.18 (d, J = 7.2 Hz, 2H), 3.14 (s, 3H), 1.82-1.95 (m, 1H), 0.95 (d, J = 6.8 Hz, 6H). |
| 15 | 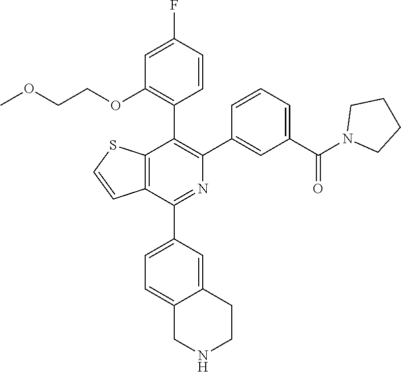 [SMILES: COCCOc1cc(F)ccc1-c1c(-c2cccc(C(=O)N3CCCC3)c2)nc(-c2ccc3c(c2)CCNC3)c2ccsc12] | LCMS m/z [M + 1]: 608 ¹H NMR (400 MHz, CD₃OD) δ7.94 (d, J = 5.6 Hz, 1H), 7.85-7.87 (m, 2H), 7.69-7.73 (m, 2H), 7.55 (s, 1H), 7.46-7.51 (m, 3H), 7.32 (t, J = 1.6 Hz, 1H), 6.93- 6.96 (m, 1H), 6.77-6.82 (m, 1H), 4.51 (s, 2H), 4.01- 4.07 (m, 1H), 3.89-3.91 (m, 1H), 3.60 (t, J = 6.4 Hz, 2H), 3.53-3.55 (m, 2H), 3.46-3.50 (m, 2H), 3.27-3.31 (m, 2H), 3.06 (t, J = 6.8 Hz, 2H), 1.92-1.97 (m, 2H), 1.83-1.88 (m, 2H). |
| 16 | 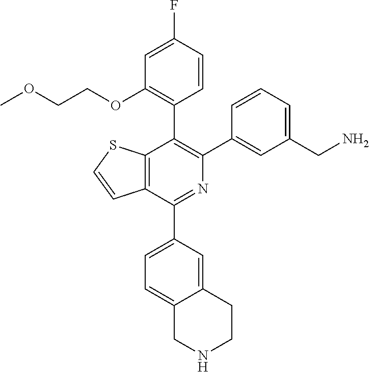 [SMILES: COCCOc1cc(F)ccc1-c1c(-c2cccc(CN)c2)nc(-c2ccc3c(c2)CCNC3)c2ccsc12] | LCMS m/z [M + 1]: 540 ¹H NMR (400 MHz, CD₃OD) δ 7.74-7.78 (m, 3H), 7.63-7.65 (m, 2H), 7.38-7.41 (m, 2H), 7.34 (d, J = 8 Hz, 1H), 7.27 (t, J = 7.6 Hz, 1H), 7.21 (dd, J = 8.4, 6.8 Hz, 1H), 6.89 (dd, J = 11.2, 2.4 Hz, 1H), 6.72- 6.74 (m, 1H), 4.37 (s, 2H), 4.00-4.04 (m, 3H), 3.88- 3.89 (m, 1H), 3.45-3.49 (m, 4H), 3.18-3.42 (m, 2H), 3.13 (s, 3H). |
| 17 | 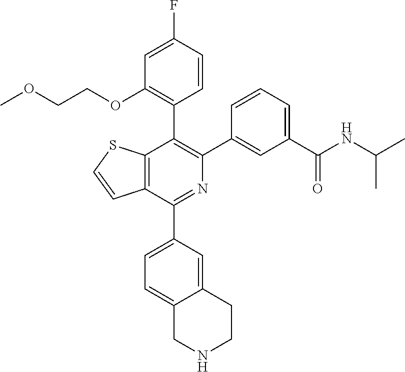 [SMILES: COCCOc1cc(F)ccc1-c1c(-c2cccc(C(=O)NC(C)C)c2)nc(-c2ccc3c(c2)CCNC3)c2ccsc12] | LCMS m/z [M + 1]: 596 ¹H NMR (400 MHz, CD₃OD) δ 8.01 (s, 1H), 7.94 (d, J = 5.6 Hz, 1H), 7.86-7.87 (m, 2H), 7.77 (d, J = 8.0 Hz, 1H), 7.73 (d, J = 5.6 Hz, 1H), 7.58 (d, J = 8.0 Hz, 1H), 7.51 (d, J = 8.0 Hz, 1H), 7.31-7.38 (m, 2H), 6.90 (dd, J = 11.2, 2.0 Hz, 1H), 6.77-6.80 (m, 1H), 4.51 (s, 2H), 4.10-4.20 (m, 1H), 4.00-4.08 (m, 1H), 3.80-3.88 (m, 1H), 3.60 (t, J = 6.4 Hz, 2H), 3.45-3.48 (m, 2H), 3.14 (s, 3H), 1.23 (d, J = 4.0 Hz, 6H). |
| 18 | 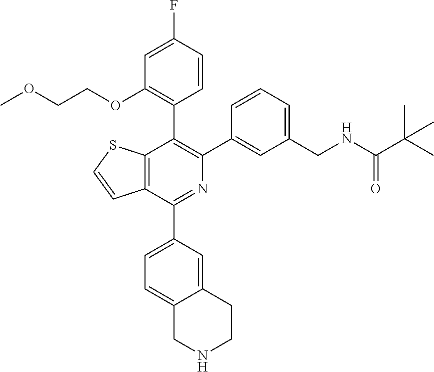 [SMILES: COCCOc1cc(F)ccc1-c1c(-c2cccc(CNC(=O)C(C)(C)C)c2)nc(-c2ccc3c(c2)CCNC3)c2ccsc12] | LCMS m/z [M + 1]: 624 ¹H NMR (400 MHz, CD₃OD) δ 7.94 (d, J = 5.6 Hz, 1H), 7.84-7.86 (m, 2H), 7.72 (d, J = 5.6 Hz, 1H), 7.49-7.52 (m, 2H), 7.24-7.31 (m, 4H), 6.90-6.93 (m, 1H), 6.76 (t, J = 6.4 Hz, 1H), 4.52 (s, 2H), 4.32 (d, J = 7.2 Hz, 2H), 4.01-4.05 (m, 1H), 3.86-3.90 (m, 1H), 3.55-3.65 (m, 2H), 3.46-3.49 (m, 2H), 3.14 (s, 3H), 1.19 (s, 9H). |
| 19 | 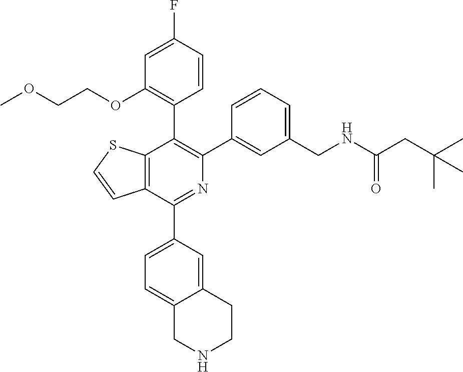 [SMILES: COCCOc1cc(F)ccc1-c1c(-c2cccc(CNC(=O)CC(C)(C)C)c2)nc(-c2ccc3c(c2)CCNC3)c2ccsc12] | LCMS m/z [M + 1]: 638 ¹H NMR (400 MHz, CD₃OD) δ 7.83-7.85 (m, 3H), 7.67 (d, J = 5.6 Hz, 1H), 7.46-7.50 (m, 2H), 7.34 (d, J = 6.8 Hz, 1H), 7.22-7.27 (m, 3H), 6.90 (dd, J = 11.2, 2.0 Hz, 1H), 6.74-6.77 (m, 1H), 4.50 (s, 2H), 4.30 (d, J = 6.0 Hz, 2H), 4.01-4.04 (m, 1H), 3.83-3.87 (m, 1H), 3.55-3.65 (m, 2H), 3.42-3.51 (m, 2H), 3.13 (s, 3H), 2.08 (s, 2H), 1.00 (s, 9H). |
| 20 | 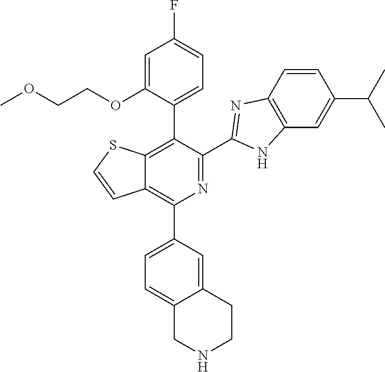 [SMILES: COCCOc1cc(F)ccc1-c1c(-c2nc3ccc(C(C)C)cc3[nH]2)nc(-c2ccc3c(c2)CCNC3)c2ccsc12] | LCMS m/z [M + 1]: 593.2 ¹H NMR (400 MHz, CD₃OD) δ 8.09 (d, J = 5.3 Hz, 1H), 7.98-8.01 (m, 2H), 7.86 (d, J = 5.5 Hz, 1H), 7.76-7.83 (m, 1H), 7.66 (d, J = 8.5 Hz, 1H), 7.59 (s, 1H), 7.51 (d, J = 8.4 Hz, 2H), 6.98-7.08 (m, 2H), 4.53 (s, 2H), 3.73-3.95 (m, 2H), 3.62 (t, J = 6.1 Hz, 2H), 3.17-3.29 (m, 3H), 3.05-3.16 (m, 1H), 2.91 (s, 3H), 1.33 (d, J = 6.9 Hz, 6H). |
| 21 | 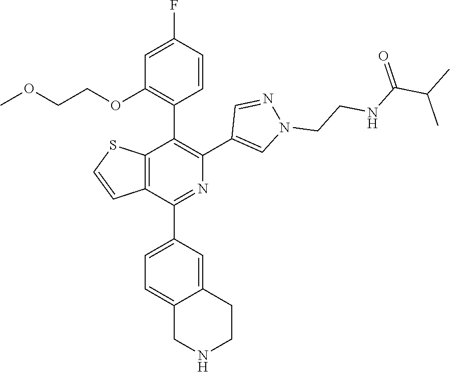 [SMILES: COCCOc1cc(F)ccc1-c1c(-c2cnn(CCNC(=O)C(C)C)c2)nc(-c2ccc3c(c2)CCNC3)c2ccsc12] | LCMS m/z [M + 1]: 614.3 ¹H NMR (400 MHz, CD₃OD) δ 7.85-7.87 (m, 2H), 7.75 (d, J = 5.6 Hz, 1H), 7.63-7.65 (m, 2H), 7.48 (d, J = 7.6 Hz, 1H), 7.36 (s, 1H), 7.29-7.33 (m, 1H), 7.07 (dd, J = 11.2, 2.0 Hz, 1H), 6.90-6.94 (m, 1H), 4.51 (s, 2H), 4.18 (t, J = 6.0 Hz, 2H), 3.99-4.09 (m, 2H), 3.61 (t, J = 6.0 Hz, 2H), 3.51 (t, J = 6.0 Hz, 2H), 3.51 (t, J = 4.4 Hz, 2H), 3.28-3.31 (m, 2H), 3.05 (s, 3H), 2.31- 2.38 (m, 1H), 1.05 (d, J = 6.8 Hz, 6H). |
| 22 | 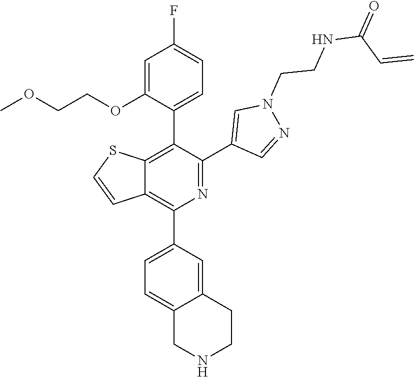 [SMILES: C=CC(=O)NCCn1cc(-c2nc(-c3ccc4c(c3)CCNC4)c3ccsc3c2-c2ccc(F)cc2OCCOC)cn1] | LCMS m/z [M + 1]: 598.2 ¹H NMR (400 MHz, CD₃OD) δ 7.86-7.88 (m, 2H), 7.82 (d, J = 5.6 Hz, 1H), 7.67 (d, J = 5.6 Hz, 1H), 7.63 (s, 1H), 7.51 (d, J = 8.0 Hz, 1H), 7.47 (s, 1H), 7.07 (t, J = 7.2 Hz, 1H), 7.05-7.08 (m, 1H), 6.89-6.94 (m, 1H), 6.12-6.22 (m, 2H), 5.67(dd, J = 8.4, 3.6 Hz, 1H), 4.53 (s, 2H), 4.23 (t, J = 6.0 Hz, 2H), 3.98-4.10 (m, 2H), 3.64-3.64 (m, 4H), 3.43 (t, J = 4.4 Hz, 2H), 3.27- 3.31 (m, 2H), 3.07 (s, 3H). |
| 23 | 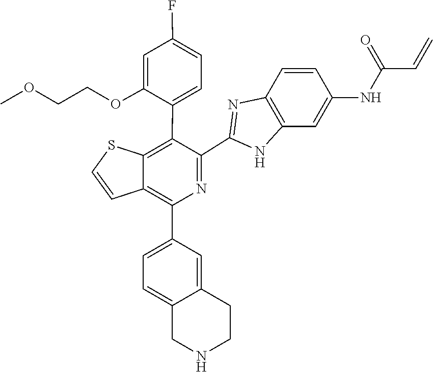 [SMILES: C=CC(=O)Nc1ccc2nc(-c3nc(-c4ccc5c(c4)CCNC5)c4ccsc4c3-c3ccc(F)cc3OCCOC)[nH]c2c1] | LCMS m/z [M + 1]: 620.2 ¹H NMR (400 MHz, CD₃OD) δ 8.48 (s, 1H), 8.06 (d, J = 5.6 Hz, 1H), 7.99-8.01 (m, 2H), 7.85 (d, J = 5.4 Hz, 1H), 7.77 (t, J = 7.6 Hz, 1H), 7.66 (d, J = 8.9 Hz, 1H), 7.49-7.53 (m, 2H), 6.97-7.04 (m, 2H), 6.38-6.52 (m, 2H), 5.83 (dd, J = 9.3, 2.3 Hz, 1H), 4.53 (s, 2H), 3.88-3.95 (m, 1H), 3.72-3.80 (m, 1H), 3.61 (t, J = 6.3 Hz 2H), 3.20-3.30 (m, 4H), 2.96 (s, 3H). |
| 24 | 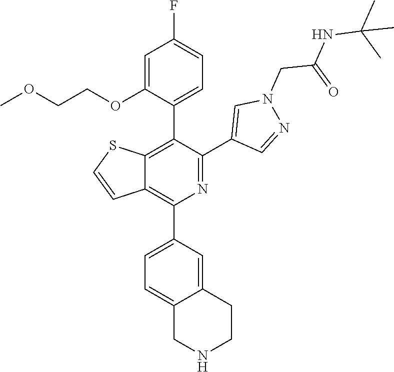 [SMILES: COCCOc1cc(F)ccc1-c1c(-c2cnn(CC(=O)NC(C)(C)C)c2)nc(-c2ccc3c(c2)CCNC3)c2ccsc12] | LCMS m/z [M + 1]: 614.3 ¹H NMR (400 MHz, CD₃OD) δ 7.87-7.89 (m, 2H), 7.78 (d, J = 5.6 Hz, 1H), 7.66-7.70 (m, 2H), 7.47-7.51 (m, 2H), 7.35-7.39 (m, 1H), 7.07 (dd, J = 10.8, 2.0 Hz, 1H), 6.91-6.96 (m, 1H), 4.71 (s, 2H), 4.52 (s, 2H), 4.01-4.11 (m, 2H), 3.62 (t, J = 6.0 Hz, 2H), 3.44 (t, J = 4.4 Hz, 2H), 3.29-3.33 (m, 2H), 3.07 (s, 3H), 1.32 (s, 9H). |
| 25 | 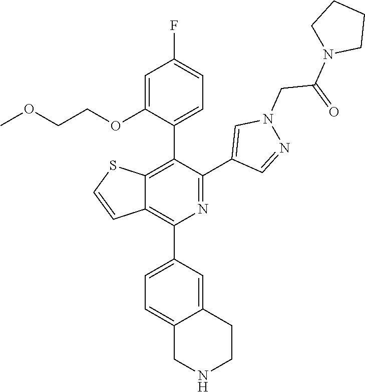 [SMILES: COCCOc1cc(F)ccc1-c1c(-c2cnn(CC(=O)N3CCCC3)c2)nc(-c2ccc3c(c2)CCNC3)c2ccsc12] | LCMS m/z [M + 1]: 612.3 ¹H NMR (400 MHz, CD₃OD) δ 7.86-7.88 (m, 2H), 7.78 (d, J = 5.6 Hz, 1H), 7.63-7.67 (m, 2H), 7.48-7.50 (m, 2H), 7.35-7.39 (m, 1H), 7.07 (dd, J = 11.2, 2.0 Hz, 1H), 6.91-6.95 (m, 1H), 4.99 (s, 2H), 4.52 (s, 2H), 4.00-4.11 (m, 2H), 3.62 (t, J = 6.0 Hz, 2H), 3.53 (t, J = 6.8 Hz, 2H), 3.43-3.46 (m, 4H), 3.29-3.33 (m, 2H), 3.07 (s, 3H),. 1.99-2.06 (m, 2H), 1.87-1.94 (m, 2H). |
| 26 | 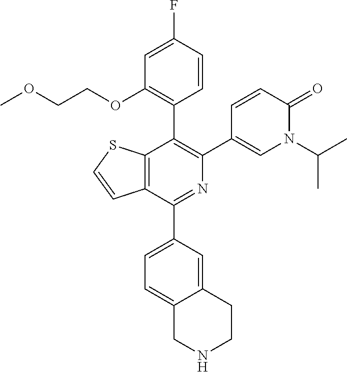 [SMILES: COCCOc1cc(F)ccc1-c1c(-c2ccc(=O)n(C(C)C)c2)nc(-c2ccc3c(c2)CCNC3)c2ccsc12] | LCMS m/z [M + 1]: 570.2 ¹H NMR (400 MHz, CD₃OD) δ 7.90 (dd, J = 9.4, 2.5 Hz, 1H), 7.84 (d, J = 8.1 Hz, 2H), 7.80 (d, J = 5.6 Hz, 1H), 7.66 (d, J = 5.6 Hz, 1H), 7.54 (d, J = 2.4 Hz, 1H), 7.46 (d, J = 7.7 Hz, 1H), 7.41 (t, J = 6.7 Hz, 1H), 7.03 (dd, J = 11.0, 2.2 Hz, 1H), 6.89 (dt, J = 8.2, 2.2 Hz, 1H), 6.54 (d, J = 9.3 Hz, 1H), 5.05-5.09 (m, 1H), 4.50 (s, 2H), 4.02-4.05 (m, 2H), 3.59 (t, J = 6.3 Hz, 2H), 3.45-3.48 (m, 2H), 3.28 (t, J = 6.1 Hz, 2H), 3.10 (s, 3H), 3.13 (d, J = 6.9 Hz, 3H), 1.11 (d, J = 6.8 Hz, 3H). |
| 27 | 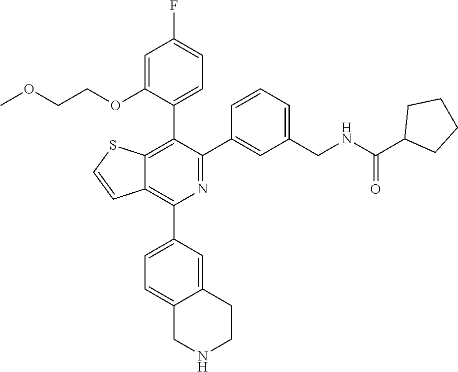 [SMILES: COCCOc1cc(F)ccc1-c1c(-c2cccc(CNC(=O)C3CCCC3)c2)nc(-c2ccc3c(c2)CCNC3)c2ccsc12] | LCMS m/z [M + 1]: 636.2 ¹H NMR (400 MHz, CD₃OD) δ 7.81-7.89 (m, 3H), 7.68 (d, J = 5.5 Hz, 1H), 7.46-7.51 (m, 2H), 7.30-7.35 (m, 1H), 7.21-7.29 (m, 3H), 6.90 (dd, J = 11.1, 1.9 Hz, 1H)), 6.75 (td, J = 8.4, 2.0 Hz, 1H), 4.51 (s, 2H), 4.31 (d, J = 4.1 Hz, 2H), 3.99-4.06 (m, 1H), 3.81-3.88 (m, 1H), 3.60 (t, J = 6.1 Hz, 2H), 3.43-3.49 (m, 2H), 3.13 (s, 3H), 2.58-2.69 (m, 1H), 1.59-1.91 (m, 8H). |
| 28 | 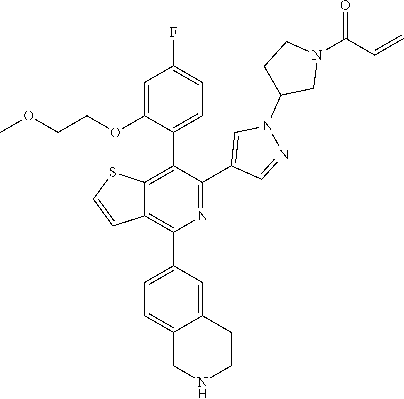 [SMILES: C=CC(=O)N1CCC(n2cc(-c3nc(-c4ccc5c(c4)CCNC5)c4ccsc4c3-c3ccc(F)cc3OCCOC)cn2)C1] | LCMS m/z [M + 1]: 624.3 ¹H NMR (400 MHz, CD₃OD) δ 7.83-7.88 (m, 2H), 7.80 (t, J = 5.2 Hz, 1H), 7.60-7.67 (m, 2H), 7.47-7.54 (m, 2H), 7.30-7.37 (m, 1H), 7.01-7.09 (m, 1H), 6.92 (t, J = 8.1 Hz, 1H), 6.50-6.65 (m, 1H), 6.26-6.34 (m, 1H), 5.77 (t, J = 11.2 Hz, 1H), 4.52 (s, 2H), 4.02-4.09 (m, 2H), 3.90-3.99 (m, 2H), 3.77-3.88 (m, 2H), 3.65- 3.74 (m, 1H), 3.61 (t, J = 6.3 Hz, 2H), 3.39-3.44 (m, 2H), 3.06 (s, 3H), 2.32-2.53 (m, 2H). |
| 29 | 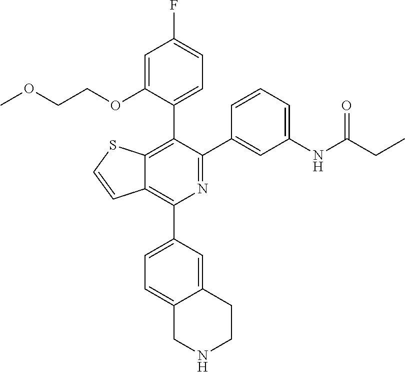 [SMILES: CCC(=O)Nc1cccc(-c2nc(-c3ccc4c(c3)CCNC4)c3ccsc3c2-c2ccc(F)cc2OCCOC)c1] | LCMS m/z [M + 1]: 582.2 ¹H NMR (400 MHz, CD₃OD) δ 7.98 (s, 1H), 7.83- 7.90 (m, 3H), 7.69 (d, J = 5.6 Hz, 1H), 7.49 (d, J = 7.9 Hz, 1H), 7.40 (d, J = 8.1 Hz, 1H), 7.29 (dd, J = 8.3, 6.7 Hz, 1H), 7.19 (t, J = 7.9 Hz, 1H), 7.11 (d, J = 7.6 Hz, 1H), 6.89 (dd, J = 11.0, 2.2 Hz, 1H), 6.77 (td, J = 8.3, 2.4 Hz, 1H), 4.51 (s, 2H), 3.99-4.08 (m, 1H), 3.79-3.92 (m, 1H), 3.60 (t, J = 6.3 Hz, 2H), 3.44-3.51 (m, 2H), 3.26-3.29 (m, 2H), 3.13 (s, 3H), 2.37 (q, J = 7.6 Hz, 2H), 1.19 (t, J = 7.6 Hz, 3H). |
| 30 | 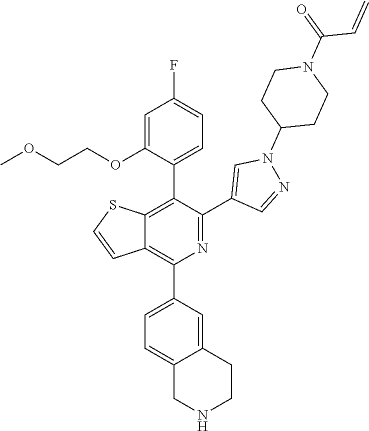 [SMILES: C=CC(=O)N1CCC(n2cc(-c3nc(-c4ccc5c(c4)CCNC5)c4ccsc4c3-c3ccc(F)cc3OCCOC)cn2)CC1] | LCMS m/z [M + 1]: 638.3 ¹H NMR (400 MHz, CD₃OD) δ 7.83-7.85 (m, 2H), 7.74 (d, J = 5.6 Hz, 1H), 7.68 (s, 1H), 7.63 (d, J = 5.6 Hz, 1H), 7.47 (d, J = 8.0 Hz, 1H), 7.39 (s, 1H), 7.32- 7.35 (m, 1H), 7.06 (dd, J = 11.2, 2.0 Hz, 1H), 6.90- 6.95 (m, 1H), 6.76-6.83 (m, 1H), 6.21 (dd, J = 16.4, 1.6 Hz, 1H), 5.76 (dd, J = 10.8, 1.6 Hz, 1H), 4.61- 4.64 (m, 1H), 4.50 (s, 2H), 4.38-4.46 (m, 1H), 4.19- 4.22 (m, 1H), 4.04-4.09 (m, 1H), 3.93-3.98 (m, 1H), 3.60 (t, J = 6.0 Hz, 2H), 3.41 (t, J = 4.4 Hz, 2H), 3.27- 3.31 (m, 3H), 3.05 (s, 3H), 2.87-2.94 (m, 1H), 2.02- 2.13 (m, 2H), 1.78-1.92 (m, 2H). |
| 31 | 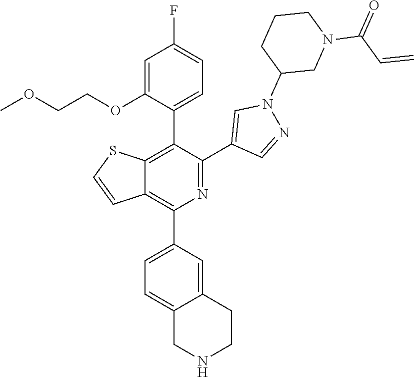 [SMILES: C=CC(=O)N1CCCC(n2cc(-c3nc(-c4ccc5c(c4)CCNC5)c4ccsc4c3-c3ccc(F)cc3OCCOC)cn2)C1] | LCMS m/z [M + 1]: 638.2 ¹H NMR (400 MHz, CD₃OD) δ 7.61-8.02 (m, 5H), 7.43-7.55 (m, 2H), 7.32-7.38 (m, 1H), 7.08 (d, J = 11.1 Hz, 1H), 6.93 (t, J = 8.3 Hz, 1H), 6.64-6.83 (m, 1H), 6.12-6.23 (m, 1H), 5.69-5.78 (m, 1H), 4.53 (s, 2H), 4.22-4.38 (m, 1H), 3.83-4.20 (m, 4H), 3.61 (t, J = 6.3 Hz, 3H), 3.32-3.50 (m, 3H), 3.09-3.28 (m, 2H), 3.10 (s, 3H), 2.02-2.25 (m, 2H), 1.76-1.97 (m, 1H), 1.54-1.70 (m, 1H). |
| 32 | 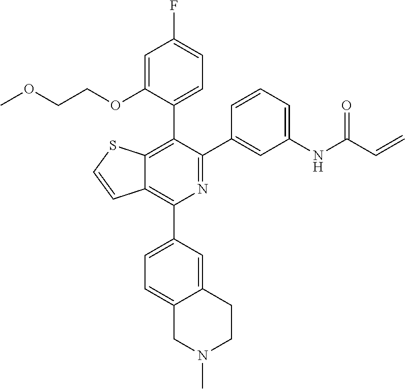 [SMILES: C=CC(=O)Nc1cccc(-c2nc(-c3ccc4c(c3)CCN(C)C4)c3ccsc3c2-c2ccc(F)cc2OCCOC)c1] | LCMS m/z [M + 1]: 594.2 ¹H NMR (400 MHz, CD₃OD) δ 8.07 (s, 1H), 7.86- 7.90 (m, 3H), 7.70 (d, J = 5.6 Hz, 1H), 7.47 (d, J = 8.4 Hz, 2H), 7.30 (t, J = 7.5 Hz, 1H), 7.22 (t, J = 7.9 Hz, 1H), 7.13 (d, J = 7.7 Hz, 1H), 6.89 (dd, J = 11.2, 2.2 Hz, 1H), 6.77 (dt, J = 8.4, 2.3 Hz, 1H), 6.32-6.45 (m, 2H), 5.77 (dd, J = 9.5, 2.1 Hz, 1H), 4.50-4.69 (m, 2H), 4.0-4.05 (m, 1H), 3.83-3.88 (m, 1H), 3.45-3.61 (m, 4H), 3.37 (s, 2H), 3.12 (s, 3H), 3.11 (s, 3H). |
| 33 | 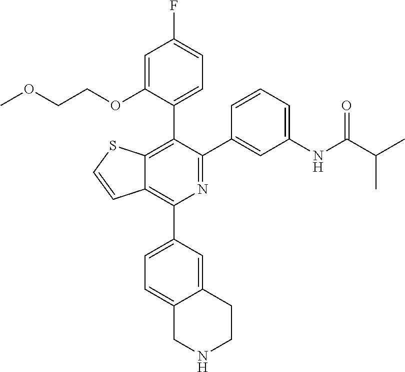 [SMILES: COCCOc1cc(F)ccc1-c1c(-c2cccc(NC(=O)C(C)C)c2)nc(-c2ccc3c(c2)CCNC3)c2ccsc12] | LCMS m/z [M + 1]: 596.3 ¹H NMR (400 MHz, CD₃OD) δ 7.98 (s, 1H), 7.92 (d, J = 5.6 Hz, 1H), 7.85 (d, J = 8.0 Hz, 1H), 7.84 (s, 1H), 7.70 (d, J = 5.6 Hz, 1H), 7.50 (d, J = 7.9 Hz, 1H), 7.41 (d, J = 7.6 Hz, 1H), 7.29 (t, J = 7.5 Hz, 1H), 7.20 (t, J = 7.8 Hz, 1H), 7.11 (d, J = 7.7 Hz, 1H), 6.89 (dd, J = 11.1, 2.2 Hz, 1H), 6.77 (dt, J = 8.3, 2.3 Hz, 1H), 4.51 (s, 2H), 4.00-4.06 (m, 1H), 3.82-3.86 (m, 1H), 3.59 (t, J = 6.3 Hz, 2H), 3.43-3.52 (m, 3H), 3.28 (t, J = 6.3 Hz, 2H), 3.13 (s, 3H), 2.57-2.64 (m, 1H), 1.18 (s, 3H), 1.17 (s, 3H). |
| 34 | 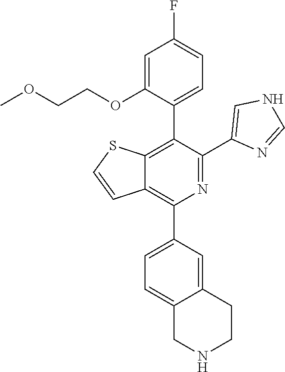 [SMILES: COCCOc1cc(F)ccc1-c1c(-c2c[nH]cn2)nc(-c2ccc3c(c2)CCNC3)c2ccsc12] | LCMS m/z [M + 1]: 501.2 ¹H NMR (400 MHz, CD₃OD) δ 7.87-7.89 (m, 2H), 7.84 (d, J = 5.6 Hz, 1H), 7.68 (d, J = 5.6 Hz, 1H), 7.62 (s, 2H), 7.52 (d, J = 8.4 Hz, 1H), 7.34-7.38 (m, 1H), 7.08 (dd, J = 11.2, 2.0 Hz, 1H), 6.91-6.96 (m, 1H), 4.53 (s, 2H), 3.98-4.10 (m, 2H), 3.62 (t, J = 6.4 Hz, 2H), 3.43 (t, J = 4.4 Hz, 2H), 3.28-3.31 (m, 2H), 3.08 (s, 3H). |
| 35 | 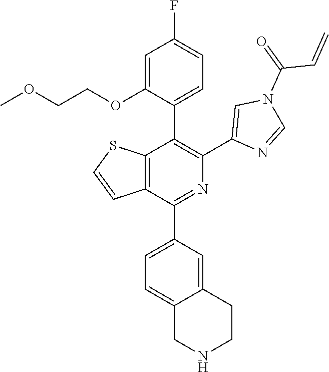 [SMILES: C=CC(=O)n1cnc(-c2nc(-c3ccc4c(c3)CCNC4)c3ccsc3c2-c2ccc(F)cc2OCCOC)c1] | LCMS m/z [M + 1]: 555.2 ¹H NMR (400 MHz, CD₃OD) δ 8.13 (s, 1H), 7.82- 7.88 (m, 2H), 7.74 (d, J = 4 Hz, 1H), 7.66 (d, J = 8 Hz, 1H), 7.45-7.65 (m, 3H), 7.36-7.39 (m, 1H),7.08 (d, J = 12 Hz, 1H), 6.96 (t, J = 8 Hz, 1H), 6.65-6.69 (m, 1H), 6.11 (d, J = 8 Hz, 1H), 4.50 (s, 2H), 4.02- 4.04 (m, 2H), 3.59-3.62 (m, 2H), 3.38-3.40 (m, 3H), 3.01 (s, 3H). |
| 36 | 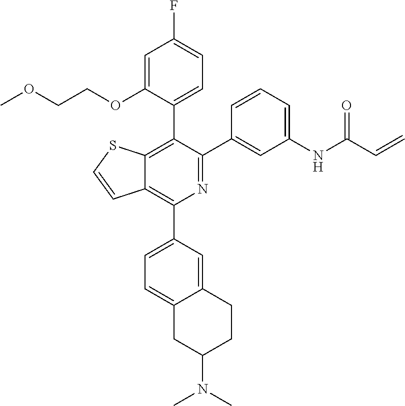 [SMILES: C=CC(=O)Nc1cccc(-c2nc(-c3ccc4c(c3)CCC(N(C)C)C4)c3ccsc3c2-c2ccc(F)cc2OCCOC)c1] | LCMS m/z [M + 1]: 622.3 ¹H NMR (400 MHz, CD₃OD) δ 8.08 (s, 1H), 7.95 (d, J = 5.6 Hz, 1H), 7.72-7.82 (m, 3H), 7.48 (d, J = 7.8 Hz, 2H), 7.23-7.33 (m, 2H), 7.14-7.18 (m, 1H), 6.90 (dd, J = 11.2, 2.2 Hz, 1H), 6.77 (dt, J = 8.4, 2.2 Hz, 1H), 6.35-6.48 (m, 2H), 5.78 (dd, J = 9.4, 2.3 Hz, 1H), 4.02-4.10 (m, 1H), 3.85-3.95 (m, 1H), 3.70-3.80 (m, 1H), 3.38-3.45 (m, 4H), 3.20-3.30 (m, 2H), 3.14 (s, 3H), 3.01 (s, 6H), 2.35-2.45 (m, 1H), 1.90-2.05 (m, 1H). |
| 37 | 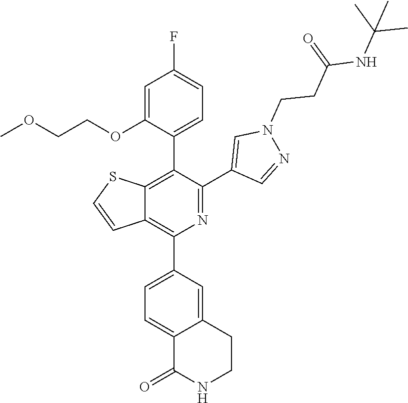 [SMILES: COCCOc1cc(F)ccc1-c1c(-c2cnn(CCC(=O)NC(C)(C)C)c2)nc(-c2ccc3c(c2)CCNC3=O)c2ccsc12] | LCMS m/z [M + 1]: 642.3 ¹H NMR (400 MHz, CD₃OD) δ 8.21 (d, J = 8.0 Hz, 1H), 7.92-7.96 (m, 3H), 7.82 (s, 1H), 7.73 (d, J = 5.6 Hz, 1H), 7.40 (s, 1H), 7.32-7.36 (m, 1H), 7.09 (dd, J = 11.2, 2.0 Hz, 1H), 6.90-6.94 (m, 1H), 4.33 (t, J = 6.8 Hz, 2H), 4.01-4.12 (m, 2H), 3.62 (t, J = 6.4 Hz, 2H), 3.45 (t, J = 4.4 Hz, 2H), 3.17 (t, J = 6.8 Hz, 2H), 3.09 (s, 3H), 2.64 (t, J = 6.8 Hz, 2H), 1.25 (s, 9H). |
| 38 | 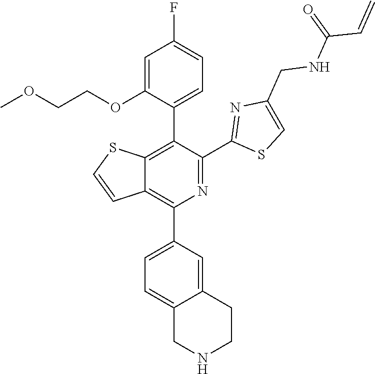 [SMILES: C=CC(=O)NCc1csc(-c2nc(-c3ccc4c(c3)CCNC4)c3ccsc3c2-c2ccc(F)cc2OCCOC)n1] | LCMS m/z [M + 1]: 601.2 ¹H NMR (400 MHz, CD₃OD) δ 7.87-7.92 (m, 2H), 7.83 (d, J = 5.6 Hz, 1H), 7.72 (d, J = 5.6 Hz, 1H), 7.46 (d, J = 7.7 Hz, 1H), 7.37 (dd, J = 8.2, 6.8 Hz, 1H), 7.29 (s, 1H), 6.81-6.97 (m, 2H), 6.22-6.26 (m, 2H), 5.70 (dd, J = 6.9, 5.1 Hz, 1H), 4.50 (s, 2H), 4.37 (s, 2H), 3.99-4.07 (m, 1H), 3.81-3.88 (m, 1H), 3.61 (t, J = 6.3 Hz, 2H), 3.00 (s, 3H). |
| 39 | 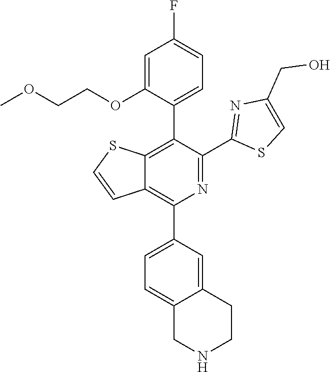 [SMILES: COCCOc1cc(F)ccc1-c1c(-c2nc(CO)cs2)nc(-c2ccc3c(c2)CCNC3)c2ccsc12] | LCMS m/z [M + 1]: 548.2 ¹H NMR (400 MHz, CD₃OD) δ 7.89 (d, J = 7.6 Hz, 2H), 7.83 (d, J = 5.5 Hz, 1H), 7.71 (d, J = 5.6 Hz, 1H), 7.45 (d, J = 8.1 Hz, 1H), 7.35-7.39 (m, 2H), 6.93 (dd, J = 11.2, 2.2 Hz, 1H), 6.86 (dt, J = 8.3, 2.3 Hz, 1H), 4.50-4.52 (m, 4H), 4.01-4.05 (m, 1H), 3.81-3.86 (m, 1H), 3.60 (t, J = 6.3 Hz, 2H), 3.35-3.40 (m, 2H), 3.25-3.30 (m, 2H), 3.01 (s, 3H). |
| 40 | 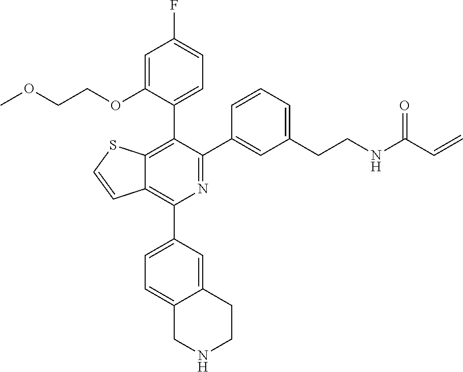 [SMILES: C=CC(=O)NCCc1cccc(-c2nc(-c3ccc4c(c3)CCNC4)c3ccsc3c2-c2ccc(F)cc2OCCOC)c1] | LCMS m/z [M + 1]: 608.2 ¹H NMR (400 MHz, CD₃OD) δ 7.93 (d, J = 5.8 Hz, 1H), 7.86 (d, J = 6.5 Hz, 1H), 7.85 (s, 1H), 7.71 (d, J = 5.6 Hz, 1H), 7.51 (d, J = 8.4 Hz, 1H), 7.40 (s, 1H), 7.35 (d, J = 7.0 Hz, 1H), 7.23-7.27 (m, 3H), 6.92 (dd, J = 11.1, 2.2 Hz, 1H), 6.76 (dd, J = 8.2, 2.1 Hz, 1H), 6.18-6.19 (m, 2H), 5.63 (dd, J = 7.0, 5.0 Hz, 1H), 4.51 (s, 2H), 4.02-4.07 (m, 1H), 3.86-3.91 (m, 1H), 3.60 (t, J = 6.4 Hz, 2H), 3.46-3.49 (m, 2H), 3.34-3.40 (m, 3H), 3.15 (s, 3H), 2.77 (t, J = 7.5 Hz, 2H). |
| 41 | 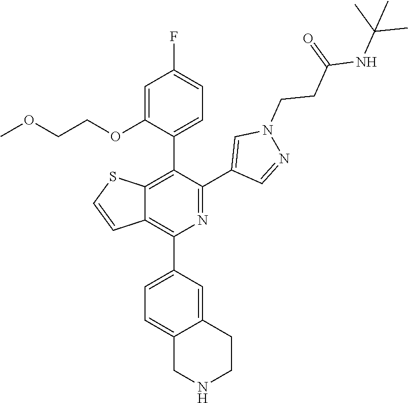 [SMILES: COCCOc1cc(F)ccc1-c1c(-c2cnn(CCC(=O)NC(C)(C)C)c2)nc(-c2ccc3c(c2)CCNC3)c2ccsc12] | LCMS m/z [M + 1]: 628.3 ¹H NMR (400 MHz, CD₃OD) δ 7.85-7.86 (m, 3H), 7.78 (d, J = 5.6 Hz, 1H), 7.65 (d, J = 5.6 Hz, 1H), 7.49 (d, J = 8.0 Hz, 1H), 7.38 (s, 1H), 7.29-7.32 (m, 1H), 7.08 (dd, J = 11.2, 2.0 Hz, 1H), 6.90-6.94 (m, 1H), 4.52 (s, 2H), 4.33 (s, 2H), 3.99-4.10 (m, 2H), 3.61 (t, J = 6.4 Hz, 2H), 3.42 (t, J = 4.0 Hz, 2H), 3.28-3.31 (m, 2H), 3.05 (s, 3H), 2.61 (s, 2H), 1.25 (s, 9H). |
| 42 | 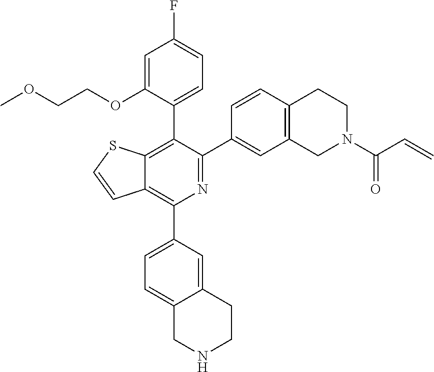 [SMILES: C=CC(=O)N1CCc2ccc(-c3nc(-c4ccc5c(c4)CCNC5)c4ccsc4c3-c3ccc(F)cc3OCCOC)cc2C1] | LCMS m/z [M + 1]: 620.3 ¹H NMR (400 MHz, CD₃OD) δ 7.83-7.84 (m, 3H), 7.66-7.67 (m, 1H), 7.47 (d, J = 7.7 Hz, 1H), 7.36-7.40 (m, 1H), 7.26 (t, J = 8.0 Hz, 2H), 7.08 (d, J = 7.7 Hz, 1H), 6.75-6.92 (m, 3H), 6.20-6.26 (m, 1H), 5.78 (d, J = 10.7 Hz, 1H), 4.70 (d, J = 6.2 Hz, 2H), 4.50 (s, 2H), 4.01-4.04 (m, 1H), 3.82-3.85 (m, 3H), 3.60 (t, J = 6.2 Hz, 2H), 3.46 (s, 2H), 3.28 (t, J = 6.3 Hz, 2H), 3.14 (s, 3H), 2.87-2.92 (m, 2H). |
| 43 | 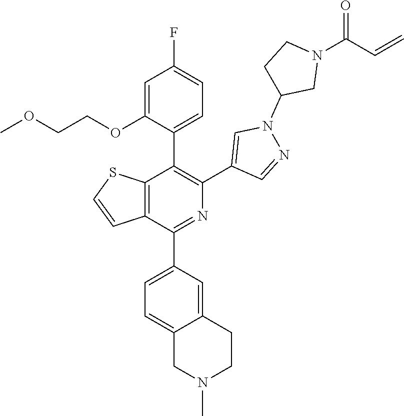 [SMILES: C=CC(=O)N1CCC(n2cc(-c3nc(-c4ccc5c(c4)CCN(C)C5)c4ccsc4c3-c3ccc(F)cc3OCCOC)cn2)C1] | LCMS m/z [M + 1]: 638.2 ¹H NMR (400 MHz, CD₃OD) δ 7.81-7.91 (m, 2H), 7.73-7.76 (m, 1H), 7.63 (d, J = 4 Hz, 1H), 7.56-7.59 (m, 1H), 7.51 (t, J = 4 Hz, 1H), 7.46 (d, J = 8 Hz, 1H), 7.31-7.35 (m, 1H), 7.04-7.06 (m, 1H), 6.89-6.93 (m, 1H), 6.51-6.64 (m, 1H), 6.27-6.32 (m, 1H), 5.74-5.80 (m, 1H), 4.97-5.02 (m, 2H), 4.49-4.69 (m, 2H),4.03- 4.07 (m, 1H), 3.94-3.96 (m, 2H), 3.48-3.88 (m, 6H), 3.40-3.41 (m, 2H), 3.13(s, 3H), 3.04(s, 3H), 2.29- 2.50 (m, 2H). |
| 44 | 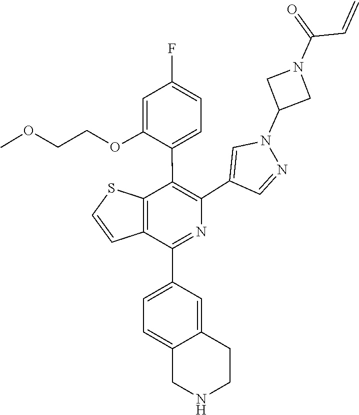 [SMILES: C=CC(=O)N1CC(n2cc(-c3nc(-c4ccc5c(c4)CCNC5)c4ccsc4c3-c3ccc(F)cc3OCCOC)cn2)C1] | LCMS m/z [M + 1]: 610.2 ¹H NMR (400 MHz, CD₃OD) δ 7.85-7.87 (m, 2H), 7.72-7.75 (m, 2H), 7.63-7.65 (m, 1H), 7.53 (d, J = 4.0 Hz, 1H), 7.48 (d, J = 8.0 Hz, 1H), 7.32-7.34 (m, 1H), 7.07 (dd, J = 11.2, 2.0 Hz, 1H), 6.90-6.95 (m, 1H), 6.25-6.40 (m, 2H), 5.78 (dd, J = 10.0, 2.0 Hz, 1H), 5.20-5.27 (m, 1H), 4.74 (t, J = 8.8 Hz, 1H), 4.47-4.57 (m, 4H), 4.30 (dd, J = 10.8, 4.8 Hz, 1H), 3.97-4.09 (m, 2H), 3.61 (t, J = 6.0 Hz, 2H), 3.42 (t, J = 4.4 Hz, 2H), 3.27-3.28 (m, 2H), 3.05 (s, 3H). |
| 45 | 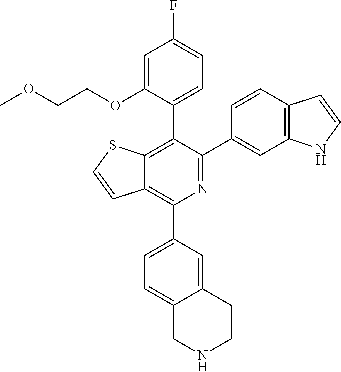 [SMILES: COCCOc1cc(F)ccc1-c1c(-c2ccc3cc[nH]c3c2)nc(-c2ccc3c(c2)CCNC3)c2ccsc12] | LCMS m/z [M + 1]: 550.2 ¹H NMR (400 MHz, CD₃OD) δ 8.07 (d, J = 5.6 Hz, 1H), 7.89-7.92 (m, 2H), 7.76 (d, J = 5.6 Hz, 1H), 7.54-7.58 (m, 3H), 7.30-7.35 (m, 2H), 7.18 (dd, J = 8.3, 1.4 Hz, 1H), 6.93 (dd, J = 11.1, 2.3 Hz, 1H), 6.77 (dt, J = 8.3, 2.3 Hz, 1H), 6.47 (d, J = 2.6 Hz, 1H), 4.54 (s, 2H), 4.01-4.06 (m, 1H), 3.81-3.86 (m, 1H), 3.61 (t, J = 6.3 Hz, 2H), 3.42-3.52 (m, 2H), 3.29-3.33 (m, 2H), 3.15 (s, 3H). |
| 46 | 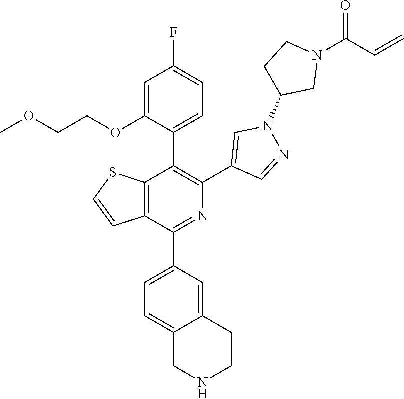 [SMILES: C=CC(=O)N1CC[C@@H](n2cc(-c3nc(-c4ccc5c(c4)CCNC5)c4ccsc4c3-c3ccc(F)cc3OCCOC)cn2)C1] | LCMS m/z [M + 1]: 624.3 ¹H NMR (400 MHz, CD₃OD) δ 7.82-7.84 (m, 2H), 7.78 (d, J = 5.6 Hz, 1H), 7.63 (d, J = 6.0 Hz, 2H), 7.46-7.50 (m, 2H), 7.29-7.34 (m, 1H), 7.00-7.06 (m, 1H), 6.88-6.92 (m, 1H), 6.49-6.63 (m, 1H), 6.25-6.32 (m, 1H), 5.73-5.80 (m, 1H), 4.96-5.02 (m, 2H), 4.50 (s, 2H), 4.02-4.07 (m, 1H), 3.90-3.96 (m, 1H), 3.83- 3.87 (m, 1H), 3.63-3.79 (m, 2H), 3.59 (t, J = 6.4 Hz, 2H), 3.38-3.41 (m, 2H), 3.27-3.31 (m, 2H), 3.05 (s, 3H), 2.23-2.50 (m, 2H). |
| 47 | 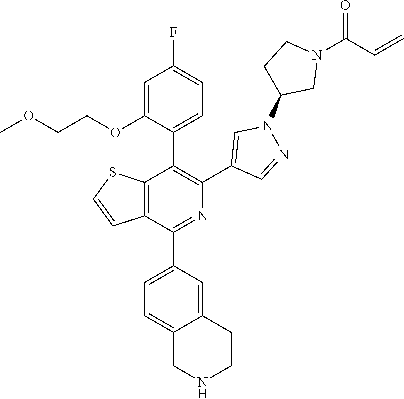 [SMILES: C=CC(=O)N1CC[C@H](n2cc(-c3nc(-c4ccc5c(c4)CCNC5)c4ccsc4c3-c3ccc(F)cc3OCCOC)cn2)C1] | LCMS m/z [M + 1]: 624.2 ¹H NMR (400 MHz, CD₃OD) δ 7.80-7.88 (m, 3H), 7.63-7.67 (m, 2H),7.47-7.54 (m, 2H), 7.28-7.38 (m, 1H), 7.01-7.08 (m, 1H), 6.87-6.95 (m, 1H), 6.49-6.66 (m, 1H), 6.25-6.34 (m, 1H), 5.82-5.73 (m, 1H), 4.96- 5.08 (m, 1H), 4.52 (s, 2H), 3.64-4.10 (m, 6H), 3.61 (t, J = 6.3 Hz, 2H), 3.42 (t, J = 4.5 Hz, 2H), 3.28-3.30 (m, 2H), 3.02-3.10 (m, 3H), 2.22-2.55 (m, 2H). |
| 47a | 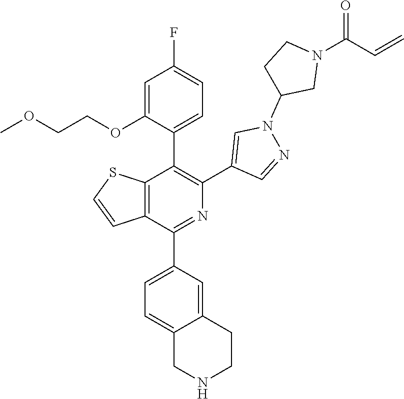 [SMILES: C=CC(=O)N1CCC(n2cc(-c3nc(-c4ccc5c(c4)CCNC5)c4ccsc4c3-c3ccc(F)cc3OCCOC)cn2)C1] | |
| 48 | 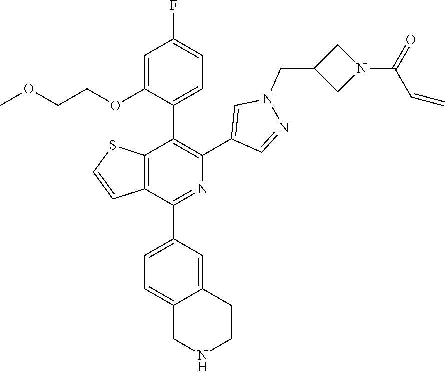 [SMILES: C=CC(=O)N1CC(Cn2cc(-c3nc(-c4ccc5c(c4)CCNC5)c4ccsc4c3-c3ccc(F)cc3OCCOC)cn2)C1] | LCMS m/z [M + 1]: 624.2 ¹H NMR (400 MHz, CD₃OD) δ 7.80-7.89 (m, 3H), 7.63-7.69 (m, 2H), 7.48-7.53 (m, 2H), 7.30-7.38 (m, 1H), 7.08 (dd, J = 11.0, 2.3 Hz, 1H), 6.93 (dt, J = 8.3, 2.3 Hz, 1H), 6.18-6.33 (m, 2H), 5.73 (dd, J = 9.7, 2.5 Hz, 1H), 4.52 (s, 2H), 4.28-4.36 (m, 3H), 3.94-4.11 (m, 4H), 3.78 (dd, J = 10.7, 5.5 Hz, 1H), 3.61 (t, J = 6.4 Hz, 2H), 3.43 (dd, J = 9.1, 4.5 Hz, 2H), 3.09-3.17 (m, 1H), 3.07 (s, 3H), 2.77 (s, 1H). |
| 49 | 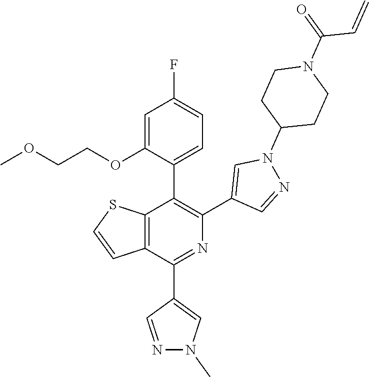 [SMILES: C=CC(=O)N1CCC(n2cc(-c3nc(-c4cnn(C)c4)c4ccsc4c3-c3ccc(F)cc3OCCOC)cn2)CC1] | LCMS m/z [M + 1]: 587.2 ¹H NMR (400 MHz, CD₃OD) δ 8.62 (s, 1H), 8.30 (s, 1H), 7.92 (d, J = 5.6 Hz, 1H), 7.81-7.83 (m, 2H), 7.31-7.35 (m, 1H), 7.21 (dd, J = 11.2, 2.4 Hz, 1H), 6.99-7.03 (m, 2H), 6.81-6.88 (m, 1H), 6.12 (dd, J = 16.8, 2.4 Hz, 1H), 5.69 (dd, J = 10.4, 2.4 Hz, 1H), 4.45-4.50 (m, 1H), 4.39-4.43 (m, 1H), 4.12-4.16 (m, 1H), 4.03-4.08 (m, 1H), 3.98 (s, 3H), 3.91-3.96 (m, 1H), 3.18-3.29 (m, 3H), 2.93 (s, 3H), 2.81 (t, J = 12.8 Hz, 1H), 1.98-2.01 (m, 2H), 1.69-1.83 (m, 2H). |
| 50 | 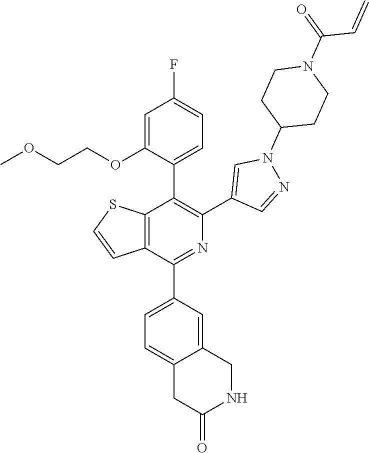 [SMILES: C=CC(=O)N1CCC(n2cc(-c3nc(-c4ccc5c(c4)CNC(=O)C5)c4ccsc4c3-c3ccc(F)cc3OCCOC)cn2)CC1] | LCMS m/z [M + 1]: 652.3 ¹H NMR (400 MHz, CD₃OD) δ 8.00 (d, J = 5.6 Hz, 1H), 7.87-7.91 (m, 3H), 7.78 (d, J = 5.6 Hz, 1H), 7.58(d, J = 8.4 Hz, 1H), 7.51(s, 1H), 7.40 (dd, J = 8.4, 6.8 Hz, 1H), 7.12 (dd, J = 10.8, 2.0 Hz, 1H), 6.90-6.96 (m, 1H), 6.82 (dd, J = 16.8, 10.4 Hz, 1H), 6.23 (dd, J = 16.8, 1.6 Hz, 1H), 5.77(d, J = 10.8 Hz, 1H), 4.58- 4.70 (m, 3H), 4.41-4.50(m , 1H), 4.18-4.30(m, 1H), 4.05-4.12(m, 1H), 3.98-4.04(m, 1H), 3.77 (s, 2H), 3.47 (t, J = 4.4 Hz, 2H), 3.14 (s, 3H), 2.88-3.00 (m , 1H), 2.12-2.20 (m, 2H), 1.89-1.98 (m, 2H). |
| 51 | 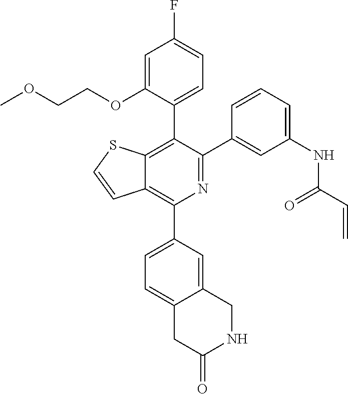 [SMILES: C=CC(=O)Nc1cccc(-c2nc(-c3ccc4c(c3)CNC(=O)C4)c3ccsc3c2-c2ccc(F)cc2OCCOC)c1] | LCMS m/z [M + 1]: 594.2 ¹H NMR (400 MHz, CD₃OD) δ 8.15 (s, 1H), 8.10 (d, J = 8 Hz, 1H), 7.88 (d, J = 4 Hz, 2H), 7.82 (d, J = 4 Hz, 1H), 7.57 (d, J = 8 Hz, 1H), 7.47-7.49 (m, 1H), 7.29-7.35 (m, 2H), 7.20(d, J = 8 Hz, 1H), 6.92-6.96 (m, 1H), 6.77-6.82(m, 1H), 6.35-6.46 (m, 2H), 5.78- 5.81 (m, 1H), 4.65 (s, 2H), 4.04-4.09 (m, 1H), 3.91- 3.96 (m, 1H), 3.74 (s, 2H), 3.48-3.53 (m, 2H), 3.16 (s, 3H). |
| 52 | 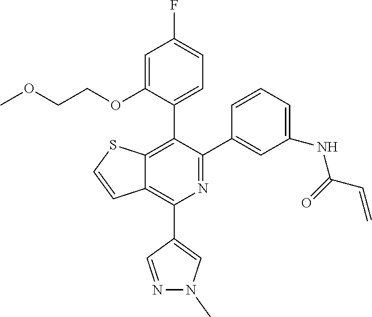 [SMILES: C=CC(=O)Nc1cccc(-c2nc(-c3cnn(C)c3)c3ccsc3c2-c2ccc(F)cc2OCCOC)c1] | LCMS m/z [M + 1]: 529.2 ¹H NMR (400 MHz, CD₃OD) δ 8.59 (s, 1H), 8.30 (s, 1H), 8.13 (d, J = 4 Hz, 2H), 8.06 (d, J = 8 Hz, 1H),7.48 (d, J = 8 Hz, 1H), 7.29-7.34 (m, 2H), 7.19 (d, J = 8 Hz, 1H), 6.91-6.94 (m, 1H), 6.76-6.81 (m, 1H), 6.36- 6.47 (m, 2H), 5.79-5.82 (m, 1H), 4.09 (s, 3H), 4.03- 4.05 (m, 1H), 3.91-3.96 (m, 1H), 3.45-3.55 (m, 2H), 3.15 (s, 3H). |
| 53 | 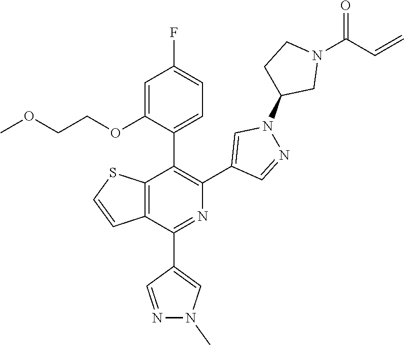 [SMILES: C=CC(=O)N1CC[C@H](n2cc(-c3nc(-c4cnn(C)c4)c4ccsc4c3-c3ccc(F)cc3OCCOC)cn2)C1] | LCMS m/z [M + 1]: 573.3 ¹H NMR (400 MHz, CD₃OD) δ: 8.55 (d, J = 3.1 Hz, 1H), 8.29 (s, 1H), 7.97 (d, J = 4.0 Hz, 2H), 7.73-7.81 (m, 1H), 7.54-7.60 (m, 1H), 7.30-7.38 (m, 1H), 6.99- 7.09 (m, 1H), 6.86-6.95 (m, 1H), 6.49-6.67 (m, 1H), 6.24-6.36 (m, 1H), 5.73-5.83 (m, 1H), 4.98-5.09 (m, 1H), 4.08 (s, 3H), 3.92-4.07 (m, 3H), 3.85-3.91 (m, 1H), 3.61-3.84 (m, 2H), 3.38-3.45 (m, 2H), 3.09 (d, J = 2.9 Hz, 3H), 2.24-2.57 (m, 2H). |
| 53a | 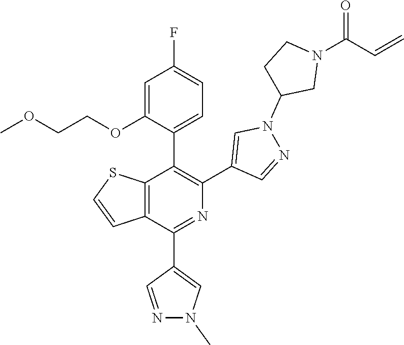 [SMILES: C=CC(=O)N1CCC(n2cc(-c3nc(-c4cnn(C)c4)c4ccsc4c3-c3ccc(F)cc3OCCOC)cn2)C1] | |
| 54 | 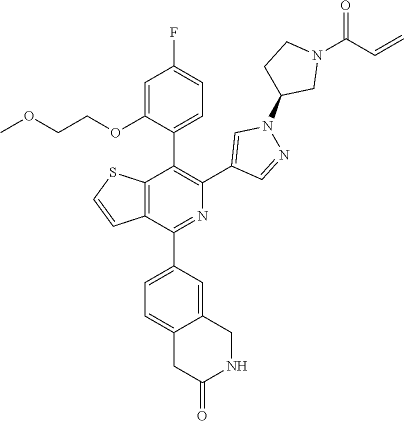 [SMILES: C=CC(=O)N1CC[C@H](n2cc(-c3nc(-c4ccc5c(c4)CNC(=O)C5)c4ccsc4c3-c3ccc(F)cc3OCCOC)cn2)C1] | LCMS m/z [M + 1]: 638.3 ¹H NMR (400 MHz, CD₃OD) δ 7.90-7.97 (m, 1H), 7.84-7.89 (m, 2H), 7.71-7.78 (m, 2H), 7.51-7.58 (m, 2H), 7.32-7.40 (m, 1H), 7.01-7.11 (m, 1H), 6.88-6.96 (m, 1H), 6.48-6.66 (m, 1H), 6.25-6.35 (m, 1H), 5.73- 5.82 (m, 1H), 4.97-5.09 (m, 1H), 4.65 (s, 2H), 3.92- 4.12 (m, 3H), 3.76-3.91 (m, 2H), 3.74 (s, 2H), 3.55- 3.70 (m, 1H), 3.39-3.46 (m, 2H), 3.10 (d, J = 3.2 Hz, 3H), 2.24-2.57 (m, 2H). |
| 54a | 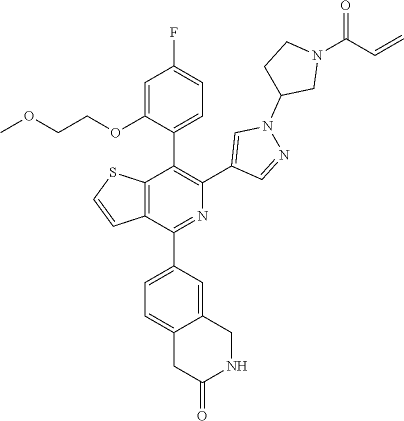 [SMILES: C=CC(=O)N1CCC(n2cc(-c3nc(-c4ccc5c(c4)CNC(=O)C5)c4ccsc4c3-c3ccc(F)cc3OCCOC)cn2)C1] | |
| 55 | 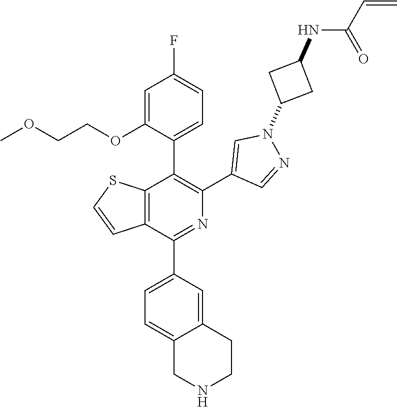 | LCMS m/z [M + 1]: 624.3 ¹H NMR (400 MHz, CD₃OD) δ 7.86 (d, J = 7.6 Hz, 2H), 7.78 (d, J = 5.6 Hz, 1H), 7.71 (s, 1H), 7.64 (d, J = 5.6 Hz, 1H), 7.46-7.50 (m, 2H), 7.34 (t, J = 6.4 Hz, 1H), 7.08 (dd, J = 10.8, 2.0 Hz, 1H), 6.93 (t, J = 6.4 Hz, 1H), 6.18-6.32 (m, 2H), 5.66-5.70 (m, 1H), 4.85- 4.92 (m, 2H), 4.52 (s, 3H), 3.95-4.10 (m, 2H), 3.61 (t, J = 6.4 Hz, 2H), 3.42 (t, J = 4.8 Hz, 2H), 3.25-3.30 (m, 2H), 3.06 (s, 3H), 2.70-2.80 (m, 2H), 2.48-2.60 (m, 2H). |
| 55a | 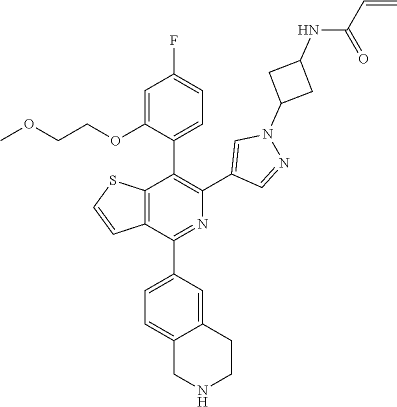 [SMILES: C=CC(=O)NC1CC(n2cc(-c3nc(-c4ccc5c(c4)CCNC5)c4ccsc4c3-c3ccc(F)cc3OCCOC)cn2)C1] | |
| 56 | 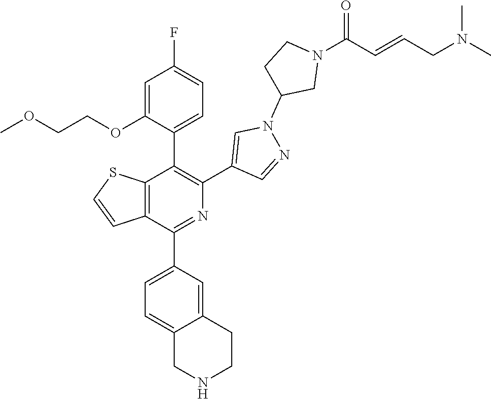 | LCMS m/z [M + 1]: 681.3 ¹H NMR (400 MHz, CD₃OD) δ 7.83-7.85 (m, 2H), 7.73-7.74 (m, 1H), 7.63-7.64 (m, 2H), 7.46-7.48 (m, 2H), 7.32-7.35 (m, 1H), 7.02-7.07 (m, 1H), 6.92 (s, 1H), 6.72-6.77 (m, 2H), 5.05 (s, 2H), 3.82-4.07 (m, 7H), 3.50-3.66 (m, 4H), 3.41 (s, 2H), 3.05 (s, 3H), 2.90-2.92 (m, 5H), 2.29-2.51 (m, 2H) |
| 57 | 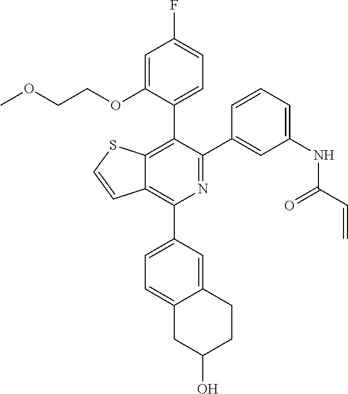 [SMILES: C=CC(=O)Nc1cccc(-c2nc(-c3ccc4c(c3)CCC(O)C4)c3ccsc3c2-c2ccc(F)cc2OCCOC)c1] | LCMS m/z [M + 1]: 595.3 ¹H NMR (400 MHz, CD₃OD) δ 8.14 (t, J = 4 Hz, 2H), 7.84(d, J = 4 Hz, 1H), 7.71-7.77 (m, 2H), 7.43-7.50 (m, 2H), 7.33(t, J = 8 Hz, 2H), 7.21(d, J = 8 Hz, 1H), 6.94-6.96 (m, 1H), 6.80(t, J = 8 Hz, 1H), 6.39-6.41 (m, 2H), 5.78-5.81 (m, 1H), 4.19-4.21 (m, 1H), 4.05- 4.09 (m, 1H), 3.94-3.97 (m, 1H), 3.49-3.54 (m, 2H), 3.17(s, 3H), 2.85-3.02 (m, 2H), 2.03-2.24 (m, 1H), 1.85-1.94 (m, 1H). |
| 58 | 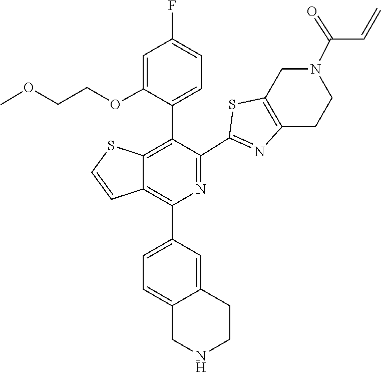 [SMILES: C=CC(=O)N1CCc2nc(-c3nc(-c4ccc5c(c4)CCNC5)c4ccsc4c3-c3ccc(F)cc3OCCOC)sc2C1] | LCMS m/z [M + 1]: 627.2 ¹H NMR (400 MHz, CD₃OD) δ 7.87- 7.92 (m, 2H), 7.81 (d, J = 5.6 Hz, 1H), 7.71 (d, J = 5.6 Hz, 1H), 7.45 (d, J = 7.9 Hz, 1H), 7.35 (dd, J = 8.2, 6.7 Hz, 1H), 6.93 (d, J = 11.0 Hz, 1H), 6.73-6.88 (m, 2H), 6.20-6.28 (m, 1H), 5.75-5.82 (m, 1H), 4.50 (s, 2H), 4.00-4.07 (m, 1H), 3.82-3.95 (m, 3H), 3.61 (t, J = 6.3 Hz, 2H), 3.00 (s, 3H), 2.66-2.75 (m, 2H). |
| 59 | 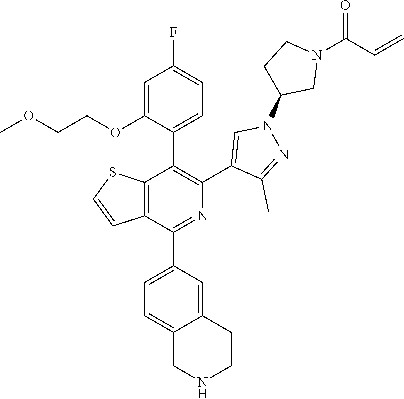 [SMILES: C=CC(=O)N1CC[C@H](n2cc(-c3nc(-c4ccc5c(c4)CCNC5)c4ccsc4c3-c3ccc(F)cc3OCCOC)c(C)n2)C1] | LCMS m/z [M + 1]: 638.2 ¹H NMR (400 MHz, CD₃OD) δ 8.01 (t, J = 4.5 Hz, 1H), 7.83 (d, J = 6.8 Hz, 2H), 7.76 (d, J = 5.6 Hz, 1H), 7.50-7.67 (m, 2H), 7.26-7.36 (m, 1H), 6.93-7.50 (m, 1H), 6.78-6.88 (m, 1H), 6.48-6.64 (m, 1H), 6.24- 6.36 (m, 1H), 5.71-5.84 (m, 1H), 4.91-5.07 (m, 2H), 4.53 (s, 2H), 3.63-4.14 (m, 6H), 3.41-3.52 (d, J = 3.6 Hz, 2H), 3.41-3.52 (m, 2H), 3.25-3.30 (m,2H), 3.15 (s, 3H), 2.26-2.52 (m, 2H), 2.20 (d, J = 9.2 Hz, 3H). |
| 59a | 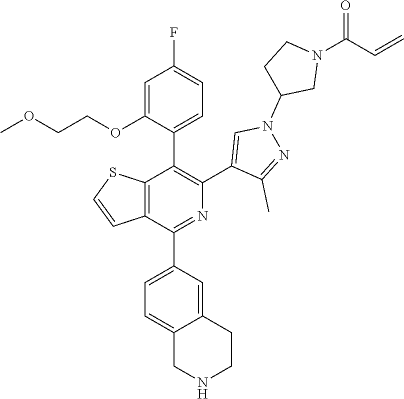 [SMILES: C=CC(=O)N1CCC(n2cc(-c3nc(-c4ccc5c(c4)CCNC5)c4ccsc4c3-c3ccc(F)cc3OCCOC)c(C)n2)C1] | |
| 60 | 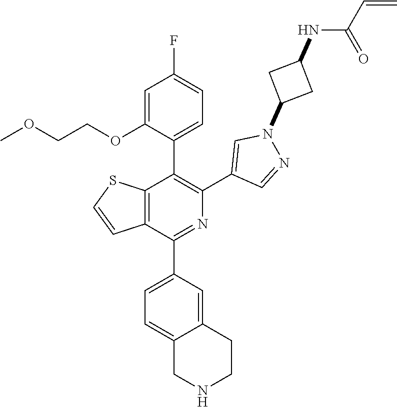 | LCMS m/z [M + 1]: 624.2 ¹H NMR (400 MHz, CD₃OD) δ 7.85 (d, J = 8.0 Hz, 2H), 7.78 (d, J = 5.6 Hz, 1H), 7.73 (s, 1H), 7.64 (d, J = 5.6 Hz, 1H), 7.49 (d, J = 8.0 Hz, 1H), 7.40 (s, 1H), 7.34 (dd, J = 8.4, 6.8 Hz, 1H), 7.07 (dd, J = 10.8, 2.0 Hz, 1H), 6.90-6.95 (m, 1H), 6.21-6.23 (m, 2H), 5.67 (dd, J = 6.8, 5.6 Hz, 1H), 4.58 (dd, J = 16.4, 8.8 Hz, 1H), 4.52 (s, 2H), 4.25-4.17 (m, 1H), 4.09-4.04 (m, 1H), 4.01-3.96 (m, 1H), 3.61 (t, J = 6.4 Hz, 2H), 3.41 (t, J = 4.8 Hz, 2H), 3.06 (s, 3H), 2.89-2.82 (m, 2H), 2.41-2.52 (m, 2H). |
| 60a | 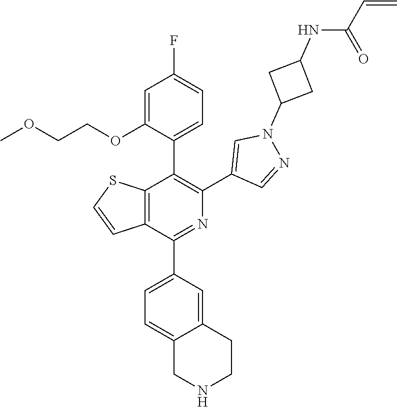 [SMILES: C=CC(=O)NC1CC(n2cc(-c3nc(-c4ccc5c(c4)CCNC5)c4ccsc4c3-c3ccc(F)cc3OCCOC)cn2)C1] | |
| 61 | 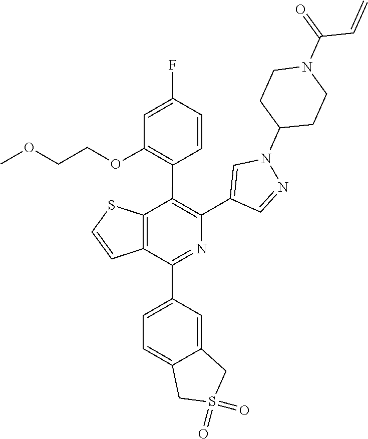 [SMILES: C=CC(=O)N1CCC(n2cc(-c3nc(-c4ccc5c(c4)CS(=O)(=O)C5)c4ccsc4c3-c3ccc(F)cc3OCCOC)cn2)CC1] | LCMS m/z [M + 1]: 673.2 ¹H NMR (400 MHz, CD₃OD) δ 7.95-8.00 (m, 3H), 7.83 (s, 1H), 7.70-7.74 (m, 2H), 7.47 (s, 1H), 7.37 (t, J = 8 Hz, 1H), 7.07-7.10 (m, 1H), 6.91-6.96 (m, 1H), 6.36-6.82 (m, 1H), 6.18-6.23 (m, 1H), 5.74-5.77 (m, 1H), 4.60-4.61 (m, 5H), 4.44-4.45 (m, 1H) ,4.19-4.22 (m, 1H), 4.06-4.10 (m, 1H), 3.43-3.44 (m, 2H), 3.28- 3.29 (m, 3H), 3.10 (s, 3H), 2.89-2.95 (m, 1H), 2.09- 2.10 (m, 2H), 1.87-1.90 (m, 2H). |
| 62 | 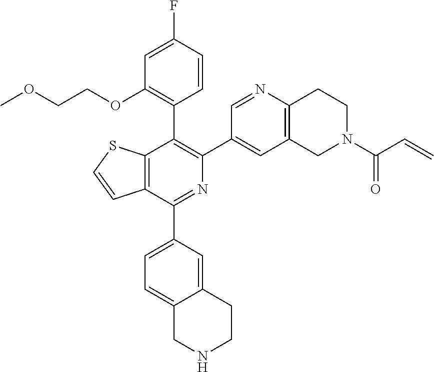 [SMILES: C=CC(=O)N1CCc2ncc(-c3nc(-c4ccc5c(c4)CCNC5)c4ccsc4c3-c3ccc(F)cc3OCCOC)cc2C1] | LCMS m/z [M + 1]: 621.2 ¹H NMR (400 MHz, CD₃OD) δ 8.49 (s, 1H), 8.12 (s, 1H), 7.85-7.88 (m, 3H), 7.71(d, J = 5.6 Hz, 1H), 7.47(d, J = 8.0 Hz, 1H), 7.42(dd, J = 8.0, 6.6 Hz, 1H), 6.70-6.96 (m, 3H), 6.20-6.32 (m, 1H), 5.81 (d, J = 10.8 Hz, 1H), 4.50 (s, 2H), 3.75-4.50 (m, 4H), 3.60 (t, J = 6.2 Hz, 2H), 3.40-3.46 (m, 2H), 3.15 (s, 3H), 3.05-3.14 (m, 2H). |
| 63 | 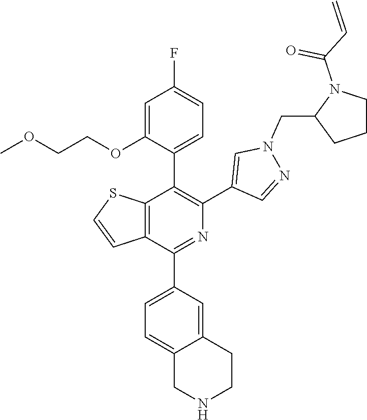 [SMILES: C=CC(=O)N1CCCC1Cn1cc(-c2nc(-c3ccc4c(c3)CCNC4)c3ccsc3c2-c2ccc(F)cc2OCCOC)cn1] | LCMS m/z [M + 1]: 638.3 ¹H NMR (400 MHz, CD₃OD) δ 7.80-7.85 (m, 2H), 7.56-7.66 (m, 2H), 7.23-7.48 (m, 4H), 7.04-7.08 (m, 1H), 6.88-6.94 (m, 1H), 6.17-6.62 (m, 2H), 5.59-5.79 (m, 1H), 4.30-4.60 (m, 5H), 3.99-4.15 (m, 3H), 3.59 (t, J = 6.4 Hz, 2H), 3.35-3.52 (m, 4H), 2.96-3.05 (m, 3H), 1.70-2.05 (m, 3H), 1.33-1.45 (m, 1H). |
| 64 | 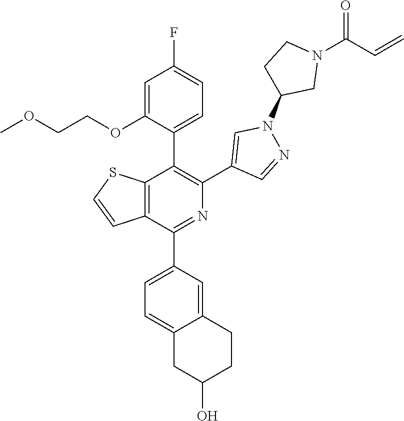 [SMILES: C=CC(=O)N1CC[C@H](n2cc(-c3nc(-c4ccc5c(c4)CCC(O)C5)c4ccsc4c3-c3ccc(F)cc3OCCOC)cn2)C1] | LCMS m/z [M + 1]: 639.3 ¹H NMR (400 MHz, CD₃OD) δ 8.02-8.09 (m, 1H), 7.89-7.81 (m, 1H), 7.78 (d, J = 5.6 Hz, 1H), 7.64-7.75 (m, 2H), 7.55-7.64 (m, 1H), 7.33-7.50 (m, 2H), 7.00- 7.11 (m, 1H), 6.86-6.96 (m, 1H), 6.48-6.65 (m, 1H), 6.24-6.35 (m, 1H), 5.72-5.82 (m, 1H), 5.05 (d, J = 18.1 Hz, 1H), 4.15-4.25 (m, 1H), 3.51-4.13 (m, 6H), 3.36-3.49 (m, 2H), 3.07-3.26 (m, 5H), 2.80-3.03 (m, 2H), 2.22-2.54 (m, 2H), 2.06-2.17 (m, 1H), 1.81- 1.98 (m, 1H). |
| 64a | 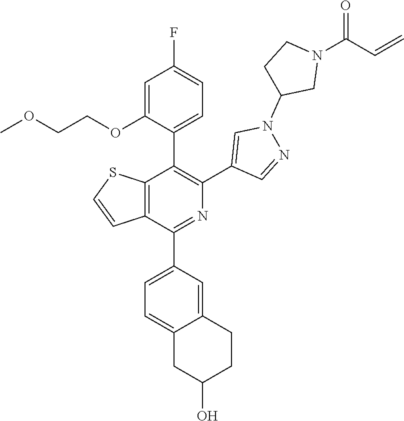 [SMILES: C=CC(=O)N1CCC(n2cc(-c3nc(-c4ccc5c(c4)CCC(O)C5)c4ccsc4c3-c3ccc(F)cc3OCCOC)cn2)C1] | |
| 65 | 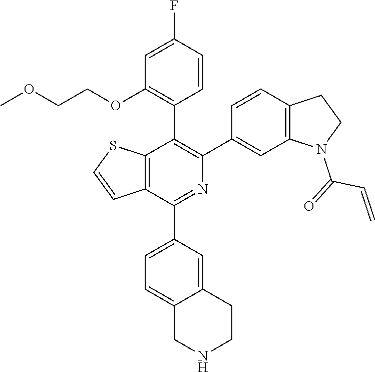 [SMILES: C=CC(=O)N1CCc2ccc(-c3nc(-c4ccc5c(c4)CCNC5)c4ccsc4c3-c3ccc(F)cc3OCCOC)cc21] | LCMS m/z [M + 1]: 606 ¹H NMR (400 MHz, CD₃OD) δ 8.47 (s, 1H), 7.82- 7.86 (m, 3H), 7.66 (d, J = 5.6 Hz, 1H), 7.47 (d, J = 7.6 Hz, 1H), 7.20-7.32 (m, 1H), 7.09-7.11 (m, 2H), 6.84-6.95 (m, 1H), 6.73-6.75 (m, 2H), 6.35-6.39 (m, 1H), 5.82-5.85 (m, 1H), 4.49 (s, 2H), 4.22-4.24 (m, 2H), 4.01-4.03 (m, 1H), 3.89-3.90 (m, 1H), 3.59 (t, J = 6.4 Hz, 2H), 3.40-3.55 (m, 2H), 3.27-3.28 (m, 2H), 3.19-3.23 (m, 2H), 3.11 (s, 3H). |
| 66 | 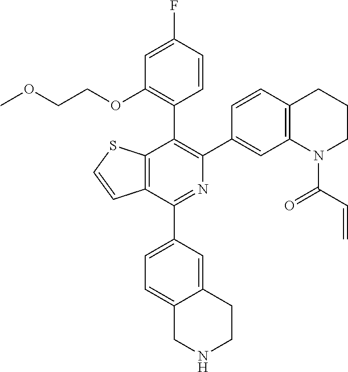 [SMILES: C=CC(=O)N1CCCc2ccc(-c3nc(-c4ccc5c(c4)CCNC5)c4ccsc4c3-c3ccc(F)cc3OCCOC)cc21] | LCMS m/z [M + 1]: 620 ¹H NMR (400 MHz, CD₃OD) δ 7.94 (d, J = 5.6 Hz, 1H), 7.84-7.86 (m, 2H), 7.72 (d, J = 5.6 Hz, 1H), 7.51 (d, J = 7.6 Hz, 1H), 7.38 (s, 1H), 7.26-7.30 (m, 2H), 7.19 (d, J = 8.0 Hz, 1H), 6.92-6.98 (m, 1H), 6.78-6.85 (m, 1H), 6.30-6.49 (m, 2H), 5.66-5.77 (m, 1H), 4.51 (s, 2H), 4.01-4.09 (m, 1H), 3.92-3.98 (m, 1H), 3.79- 3.88 (m, 2H), 3.59 (t, J = 6.4 Hz, 2H), 3.40-3.55 (m, 2H), 3.27-3.31 (m, 2H), 3.12 (s, 3H), 2.75 (t, J = 6.8 Hz, 2H), 1.95-2.05 (m, 2H). |
| 67 | 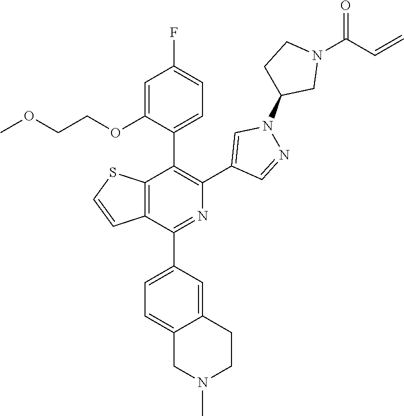 [SMILES: C=CC(=O)N1CC[C@H](n2cc(-c3nc(-c4ccc5c(c4)CCN(C)C5)c4ccsc4c3-c3ccc(F)cc3OCCOC)cn2)C1] | LCMS m/z [M + 1]: 638.3 ¹H NMR (400 MHz, CD₃OD) δ 7.86 (d, J = 6.9 Hz, 2H), 7.69-7.74 (m, 1H), 7.62 (d, J = 5.5 Hz, 1H), 7.41-7.57 (m, 4H), 7.28-7.36 (m, 1H), 7.00-7.08 (m, 1H), 6.87-6.96 (m, 1H), 6.49-6.66 (m, 1H), 6.25-6.35 (m, 1H), 5.73-5.82 (m, 1H), 4.95-5.06 (m, 1H), 4.63- 4.76 (m, 1H), 4.40-4.57 (m, 1H), 3.90-4.10 (m, 3H), 3.66-3.88 (m, 4H), 3.34-3.45 (m, 4H), 3.13 (s, 3H), 3.02-3.05 (m, 3H), 2.32-2.53 (m, 2H). |
| 67a | 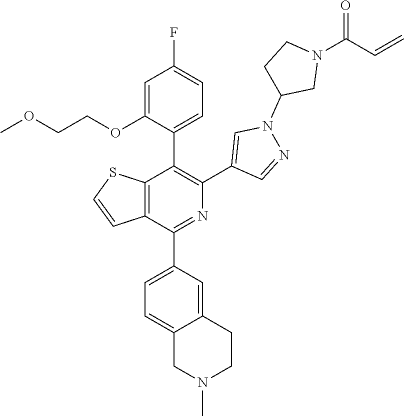 [SMILES: C=CC(=O)N1CCC(n2cc(-c3nc(-c4ccc5c(c4)CCN(C)C5)c4ccsc4c3-c3ccc(F)cc3OCCOC)cn2)C1] | |
| 68 | 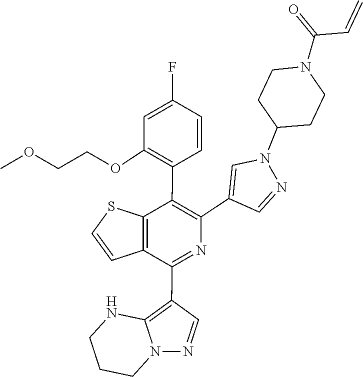 [SMILES: C=CC(=O)N1CCC(n2cc(-c3nc(-c4cnn5c4NCCC5)c4ccsc4c3-c3ccc(F)cc3OCCOC)cn2)CC1] | LCMS m/z [M + 1]: 628.3 ¹H NMR (400 MHz, CD₃OD) δ 8.01 (s, 1H), 7.96 (d, J = 5.7 Hz, 1H), 7.91 (d, J = 5.7 Hz, 1H), 7.77 (s, 1H), 7.52 (s, 1H), 7.32 (t, J = 6.4 Hz, 1H), 7.05 (d, J = 8.0 Hz, 1H), 6.90 (t, J = 6.8 Hz, 1H), 6.76-6.84 (m, 1H), 6.21 (dd, J = 16.7, 1.7 Hz, 1H), 5.76 (dd, J = 10.6, 1.7 Hz, 1H), 4.62 (d, J = 13.0 Hz, 1H), 4.40- 4.50 (m, 1H), 4.20 (t, J = 6.8 Hz, 3H), 3.90-4.08 (m, 2H), 3.40-3.52 (m, 4H), 3.25-3.38 (m, 1H), 3.13 (s, 3H), 2.91 (t, J = 11.8 Hz, 1H), 2.20-2.30 (m, 2H), 2.05-2.15 (m, 2H), 1.80-1.95 (m, 2H). |
| 69 | 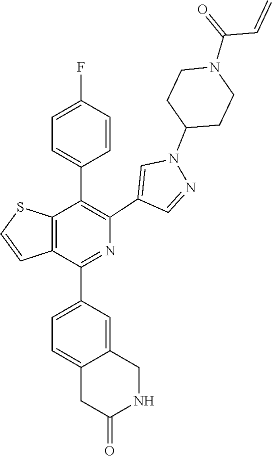 [SMILES: C=CC(=O)N1CCC(n2cc(-c3nc(-c4ccc5c(c4)CNC(=O)C5)c4ccsc4c3-c3ccc(F)cc3)cn2)CC1] | LCMS m/z [M + 1]: 578.2 ¹H NMR (400 MHz, CD₃OD) δ 7.96 (d, J = 5.6 Hz, 1H), 7.85-7.87 (m, 2H), 7.79 (s, 1H), 7.75 (d, J = 5.6 Hz, 1H), 7.48-7.55 (m, 3H), 7.39 (s, 1H), 7.33 (t, J = 8.8 Hz, 2H), 6.75-6.82 (m, 1H), 6.20 (dd, J = 16.7, 1.8 Hz, 1H), 5.75 (dd, J = 10.6, 1.9 Hz, 1H), 4.64 (s, 2H), 4.61 (d, J = 17.1 Hz, 1H), 4.40-4.50 (m, 1H), 4.20 (d, J = 13.5 Hz, 1H), 3.73 (s, 2H), 3.20-3.30 (m, 1H), 2.89 (t, J = 6.8 Hz, 1H), 2.05-2.15 (m, 2H), 1.80- 1.95 (m, 2H). |
| 70 | 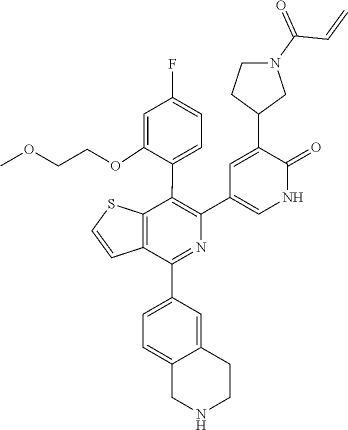 [SMILES: C=CC(=O)N1CCC(c2cc(-c3nc(-c4ccc5c(c4)CCNC5)c4ccsc4c3-c3ccc(F)cc3OCCOC)c[nH]c2=O)C1] | LCMS m/z [M + 1]: 651.3 ¹H NMR (400 MHz, CD₃OD) δ 7.81 (d, J = 9.7 Hz, 2H), 7.74 (d, J = 5.6 Hz, 1H), 7.62 (dd, J = 5.6, 1.6 Hz, 1H), 7.50-7.58 (m, 2H), 7.45 (d, J = 7.7 Hz, 1H), 7.32-7.39 (m, 1H), 6.97-7.02 (m, 1H), 6.84-6.90 (m, 1H), 6.52-6.63 (m, 1H), 6.23-6.30 (m, 1H), 5.71-5.76 (m, 1H), 4.49 (s, 2H), 3.83-4.05 (m, 3H), 3.64-3.67 (m, 1H), 3.59 (t, J = 6.3 Hz, 3H), 3.44-3.53 (m, 4H), 3.28 (t, J = 6.3 Hz, 2H), 3.10 (t, J = 2.8 Hz, 3H), 2.12- 2.28 (m, 1H), 1.81-1.88 (m, 1H). |
| 71 | 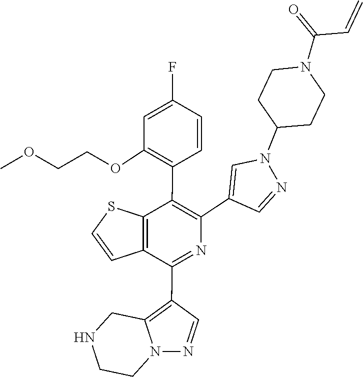 [SMILES: C=CC(=O)N1CCC(n2cc(-c3nc(-c4cnn5c4CNCC5)c4ccsc4c3-c3ccc(F)cc3OCCOC)cn2)CC1] | LCMS m/z [M + 1]: 628.2 ¹H NMR (400 MHz, CD₃OD) δ 8.35 (s, 1H), 7.87 (d, J = 5.6 Hz, 1H), 7.69 (d, J = 5.6 Hz, 1H), 7.52-7.56 (m, 1H), 7.34-7.39 (m, 1H), 7.29 (t, J = 7.4 Hz, 1H), 7.06 (dd, J = 11.2, 2.2 Hz, 1H), 6.88-6.95 (m, 1H), 6.80 (dd, J = 16.8, 10.7 Hz, 1H), 6.22 (dd, J = 16.8, 1.8 Hz, 1H), 5.77 (dd, J = 10.7, 1.9 Hz, 1H), 5.04 (s, 2H), 4.56-4.68 (m, 3H), 4.34-4.44 (m, 1H), 4.17-4.25 (m, 1H), 4.01-4.08 (m, 1H), 3.89-3.99 (m, 3H), 3.38 (t, J = 4.6 Hz, 2H), 3.01 (s, 3H), 2.90 (t, J = 12.6 Hz, 1H), 2.03-2.13 (m, 2H), 1.78-1.90 (m, 2H). |
| 72 | 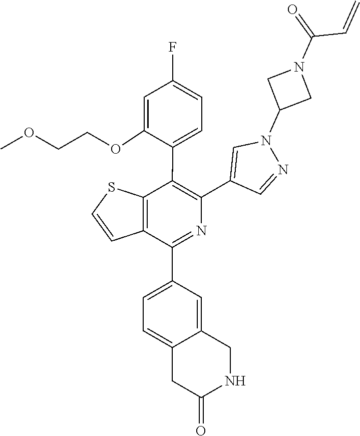 [SMILES: C=CC(=O)N1CC(n2cc(-c3nc(-c4ccc5c(c4)CNC(=O)C5)c4ccsc4c3-c3ccc(F)cc3OCCOC)cn2)C1] | LCMS m/z [M + 1]: 624.2 ¹H NMR (400 MHz, CD₃OD) δ 7.94-8.00 (m, 2H), 7.85-7.91 (m, 2H), 7.76 (d, J = 5.6 Hz, 1H), 7.52-7.60 (m, 2H), 7.37 (dd, J = 8.1, 6.8 Hz, 1H), 7.08 (dd, J = 11.0, 2.0 Hz, 1H), 6.92 (td, J = 8.3, 2.2 Hz, 1H), 6.23- 6.40 (m, 2H), 5.77 (dd, J = 9.9, 2.1 Hz, 1H), 5.23- 5.32 (m, 1H), 4.75 (t, J = 8.8 Hz, 1H), 4.65 (s, 2H), 4.53-4.59 (m, 1H), 4.45-4.53 (m, 1H), 4.26-4.34 (m, 1H), 3.97-4.12 (m, 2H), 3.73 (s, 2H), 3.45 (t, J = 4.4 Hz, 2H), 3.11 (s, 3H). |
| 73 | 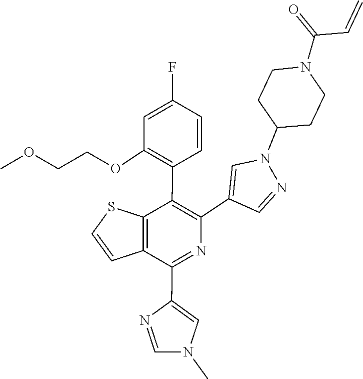 [SMILES: C=CC(=O)N1CCC(n2cc(-c3nc(-c4cn(C)cn4)c4ccsc4c3-c3ccc(F)cc3OCCOC)cn2)CC1] | LCMS m/z [M + 1]: 587.3 ¹H NMR (400 MHz, CD₃OD) δ 9.05 (s, 1H), 8.49 (s, 1H), 7.93 (d, J = 5.6 Hz, 1H), 7.86 (d, J = 5.6 Hz, 1H), 7.78-7.82 (m, 1H), 7.43 (s, 1H), 7.33 (dd, J = 8.4, 6.7 Hz, 1H), 7.09 (dd, J = 11.0, 2.2 Hz, 1H), 6.91- 6.99 (m, 1H), 6.80 (dd, J = 16.8, 10.7 Hz, 1H), 6.22 (dd, J = 16.8, 1.9 Hz, 1H), 5.77 (dd, J = 10.6, 1.9 Hz, 1H), 4.65 (d, J = 13.2 Hz, 1H), 4.36-4.47 (m, 1H), 4.22 (d, J = 13.2 Hz, 1H), 4.09 (s, 3H), 4.01-4.08 (m, 1H), 3.92-4.00 (m, 1H), 3.36 (t, J = 4.8 Hz, 2H), 2.99 (s, 3H), 2.85-2.97 (m, 1H), 2.06-2.22 (m, 2H), 1.79- 1.95 (m, 2H). |
| 74 | 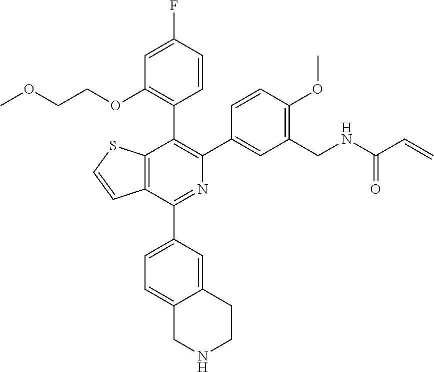 [SMILES: C=CC(=O)NCc1cc(-c2nc(-c3ccc4c(c3)CCNC4)c3ccsc3c2-c2ccc(F)cc2OCCOC)ccc1OC] | LCMS m/z [M + 1]: 624.3 ¹H NMR (400 MHz, CD₃OD) δ 7.81-7.83 (m, 2H), 7.75( d, J = 5.6 Hz, 1H), 7.62 (d, J = 5.6 Hz, 1H), 7.44-7.46 (m, 2H), 7.33-7.36 (m, 1H),7.23 (dd, J = 8.4, 6.8 Hz, 1H), 6.85-6.88 (m, 2H), 6.70-6.74 (m, 1H), 6.23-6.30 (m, 2H), 5.67(dd, J = 8.4, 3.2 Hz, 1H), 4.49 (s, 2H), 4.39 (s, 2H), 3.98-4.05 (m, 1H), 3.84- 3.85 (m, 4H), 3.6 (t, J = 6.4 Hz, 2H), 3.43 (t, J = 4.8 Hz, 2H), 3.26-3.28 (m, 2H) 3.09 (s, 3H). |
| 75 | 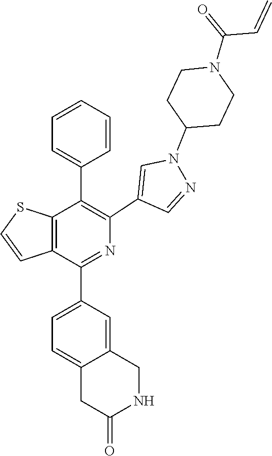 [SMILES: C=CC(=O)N1CCC(n2cc(-c3nc(-c4ccc5c(c4)CNC(=O)C5)c4ccsc4c3-c3ccccc3)cn2)CC1] | LCMS m/z [M + 1]: 560.2 ¹H NMR (400 MHz, CD₃OD) δ 7.84-7.90 (m, 3H), 7.72 (d, J = 5.6 Hz, 1H), 7.66 (s, 1H), 7.56-7.61 (m, 3H), 7.52 (d, J = 8.4 Hz, 1H), 7.43-7.49 (m, 2H), 7.34 (s, 1H), 6.78 (dd, J = 16.8, 10.6 Hz, 1H), 6.20 (dd, J = 16.8, 1.9 Hz, 1H), 5.75 (dd, J = 10.6, 1.9 Hz, 1H), 4.55-4.67 (m, 3H), 4.35-4.46 (m, 1H), 4.13-4.24 (m, 1H), 3.73 (s, 2H), 3.23-3.28 (m, 1H), 2.90 (t, J = 12.2 Hz, 1H), 2.02-2.15 (m, 2H), 1.75-1.91 (m, 2H). |
| 76 | 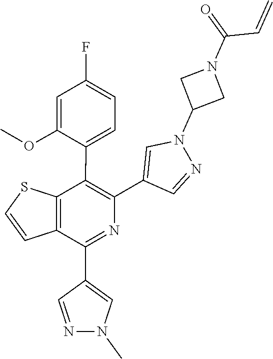 [SMILES: C=CC(=O)N1CC(n2cc(-c3nc(-c4cnn(C)c4)c4ccsc4c3-c3ccc(F)cc3OC)cn2)C1] | LCMS m/z [M + 1]: 515.2 ¹H NMR (400 MHz, CD₃OD) δ 8.59 (s, 1H), 8.30 (s, 1H), 7.97-8.05 (m, 2H), 7.95 (s, 1H), 7.59 (s, 1H), 7.33-7.38 (m, 1H), 7.03 (dd, J = 11.0, 2.2 Hz, 1H), 6.87-6.94 (m, 1H), 6.24-6.41 (m, 2H), 5.77 (dd, J = 9.9, 2.3 Hz, 1H), 5.23-5.35 (m, 1H), 4.75 (t, J = 8.8 Hz, 1H), 4.45-4.61 (m, 2H), 4.27-4.33 (m, 1H), 4.09 (s, 3H), 3.66 (s, 3H). |
| 77 | 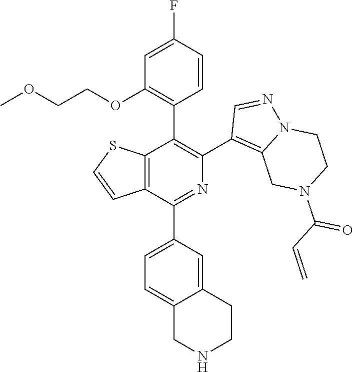 [SMILES: C=CC(=O)N1CCn2ncc(-c3nc(-c4ccc5c(c4)CCNC5)c4ccsc4c3-c3ccc(F)cc3OCCOC)c2C1] | LCMS m/z [M + 1]: 610.2 ¹H NMR (400 MHz, CD₃OD) δ 7.66-7.82 (m, 4H), 7.62 (d, J = 5.6 Hz, 1H), 7.51-7.58 (m, 1H), 7.47 (d, J = 1.7 Hz, 1H), 7.34-7.41 (m, 2H), 7.07 (dd, J = 11.0, 2.2 Hz, 1H), 6.88-6.95 (m, 1H), 6.17 (s, 1H), 5.02 (s, 1H), 4.56 (s, 1H), 4.04-4.30 (m, 8H), 3.41-3.50 (m, 1H), 3.32-3.37 (m, 2H), 3.08-3.14 (m, 2H), 3.03 (s, 3H). |
| 78 | 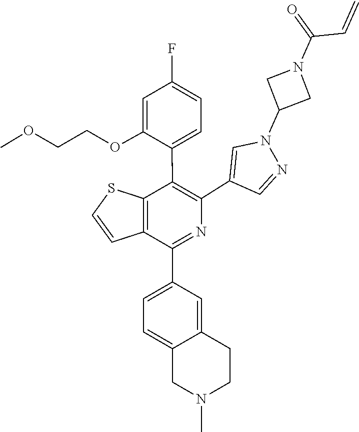 [SMILES: C=CC(=O)N1CC(n2cc(-c3nc(-c4ccc5c(c4)CCN(C)C5)c4ccsc4c3-c3ccc(F)cc3OCCOC)cn2)C1] | LCMS m/z [M + 1]: 624.3 ¹H NMR (400 MHz, CD₃OD) δ 7.85-7.90 (m, 2H), 7.75-7.79 (m, 2H), 7.65 (d, J = 5.6 Hz, 1H), 7.53 (d, J = 3.5 Hz, 1H), 7.47 (d, J = 8.5 Hz, 1H), 7.33 (dd, J = 8.3, 6.7 Hz, 1H), 7.06 (dd, J = 11.0, 2.2 Hz, 1H), 6.88-6.95 (m, 1H), 6.24-6.40 (m, 2H), 5.76 (dd, J = 9.9, 2.3 Hz, 1H), 5.19-5.28 (m, 1H), 4.73 (t, J = 8.8 Hz, 2H), 4.44-4.58 (m, 3H), 4.30 (dd, J = 10.9, 5.0 Hz, 1H), 3.96-4.10 (m, 2H), 3.74-3.90 (m, 1H), 3.46- 3.61 (m, 1H), 3.34-3.46 (m, 4H), 3.13 (s, 3H), 3.05 (s, 3H). |
| 79 | 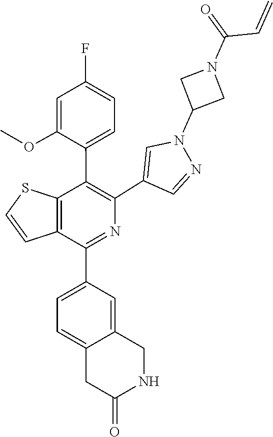 [SMILES: C=CC(=O)N1CC(n2cc(-c3nc(-c4ccc5c(c4)CNC(=O)C5)c4ccsc4c3-c3ccc(F)cc3OC)cn2)C1] | LCMS m/z [M + 1]: 580.2 ¹H NMR (400 MHz, CD₃OD) δ 7.84-7.96 (m, 4H), 7.74 (d, J = 5.6 Hz, 1H), 7.53-7.55 (m, 2H), 7.29-7.35 (m, 1H), 7.05 (dd, J = 11.0, 1.9 Hz, 1H), 6.87-6.95 (m, 1H), 6.23-6.39 (m, 2H), 5.77 (dd, J = 9.9, 2.2 Hz, 1H), 5.22-5.32 (m, 1H), 4.74 (t, J = 8.7 Hz, 1H), 4.65 (s, 2H), 4.45-4.59 (m, 2H), 4.27-4.32 (m, 1H), 3.74 (s, 2H), 3.68 (s, 3H). |
| 80 | 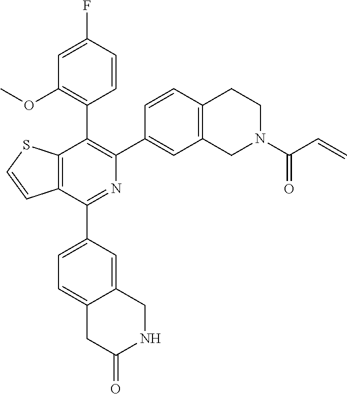 [SMILES: C=CC(=O)N1CCc2ccc(-c3nc(-c4ccc5c(c4)CNC(=O)C5)c4ccsc4c3-c3ccc(F)cc3OC)cc2C1] | LCMS m/z [M + 1]: 590.3 ¹H NMR (400 MHz, CD₃OD) δ 8.08-8.12 (m, 1H), 7.85-7.89 (m, 2H), 7.82 (d, J = 5.4 Hz, 1H), 7.57 (d, J = 8.2 Hz, 1H), 7.33-7.42 (m, 2H), 7.24-7.30 (m, 1H), 7.18 (d, J = 8.0 Hz, 1H), 6.75-6.93 (m, 3H), 6.18-6.27 (m, 1H), 5.79 (d, J = 10.6 Hz, 1H), 4.74 (s, 2H), 4.65 (s, 2H), 3.82-3.86 (m, 2H), 3.74 (s, 2H), 3.60-3.66 (m, 3H), 2.85-2.96 (m, 2H). |
| 81 | 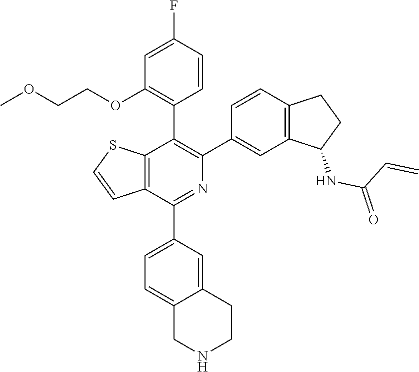 [SMILES: C=CC(=O)N[C@H]1CCc2ccc(-c3nc(-c4ccc5c(c4)CCNC5)c4ccsc4c3-c3ccc(F)cc3OCCOC)cc21] | LCMS m/z [M + 1]: 620 ¹H NMR (400 MHz, CD₃OD) δ7.97 (t, J = 4.4 Hz, 1H), 7.84-7.86 (m, 2H), 7.71 (dd, J = 5.6, 0.8 Hz, 1H), 7.52 (d, J = 7.2 Hz, 1H), 7.38-7.42 (m, 2H), 7.23-7.25 (m, 2H), 6.85-6.95 (m, 1H), 6.70-6.74 (m, 1H), 6.23-6.27 (m, 2H), 5.70-5.75 (m, 1H), 5.38-5.45 (m, 1H), 4.52 (s, 2H), 4.01-4.10 (m, 1H), 3.82-3.98 (m, 1H), 3.61 (t, J = 6.0 Hz, 2H), 3.45-3.48 (m, 2H), 3.12 (d, J = 8.8 Hz, 3H), 3.02-3.09 (m, 1H), 2.82-2.95 (m, 1H), 2.42-2.57 (m, 1H), 1.86-2.0 (m, 1H). |
| 82 | 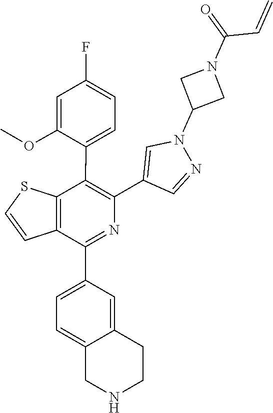 [SMILES: C=CC(=O)N1CC(n2cc(-c3nc(-c4ccc5c(c4)CCNC5)c4ccsc4c3-c3ccc(F)cc3OC)cn2)C1] | LCMS m/z [M + 1]: 566.2 ¹H NMR (400 MHz, CD₃OD) δ 7.84-7.87 (m, 2H), 7.75 (d, J = 5.6 Hz, 1H), 7.71 (s, 1H), 7.64 (d, J = 5.6 Hz, 1H), 7.43-7.55 (m, 2H), 7.25-7.39 (m, 1H), 7.04 (dd, J = 11.1, 2.2 Hz, 1H), 6.87-6.6.94 (m, 1H), 6.24- 6.39 (m, 2H), 5.77 (dd, J = 9.9, 2.3 Hz, 1H), 5.20- 5.25 (m, 1H), 4.73 (t, J = 8.7 Hz, 1H), 4.43-4.57 (m, 4H), 4.27-4.31 (m, 1H), 3.67 (s, 3H), 3.61 (t, J = 6.4 Hz, 2H), 3.25-3.37(m, 2H). |
| 83 | 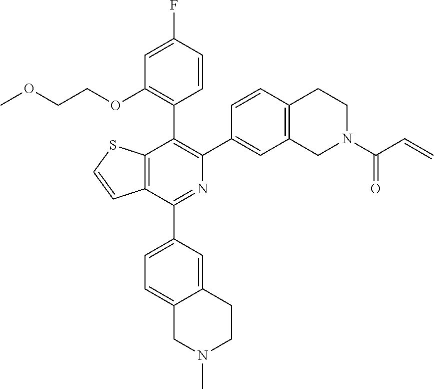 [SMILES: C=CC(=O)N1CCc2ccc(-c3nc(-c4ccc5c(c4)CCN(C)C5)c4ccsc4c3-c3ccc(F)cc3OCCOC)cc2C1] | LCMS m/z [M + 1]: 634.3 ¹H NMR (400 MHz, CD₃OD) δ 7.87-7.98 (m, 3H), 7.71 (d, J = 5.2 Hz, 1H), 7.50 (d, J = 8.2 Hz, 1H), 7.37-7.42 (m, 1H), 7.28 (t, J = 7.6 Hz, 2H), 7.12 (d, J = 7.9 Hz, 1H), 6.93 (d, J = 10.9 Hz, 1H), 6.75-6.87 (m, 2H), 6.19-6.23 (m, 1H), 5.78 (d, J = 10.8 Hz, 1H), 4.70-4.72 (m, 4H), 4.01-4.05 (m, 1H), 3.77-3.93 (m, 3H), 3.39-3.47 (m, 4H), 3.24-3.35(m, 2H), 3.11-3.18 (m, 6H), 2.85-2.94 (m, 2H). |
| 84 | 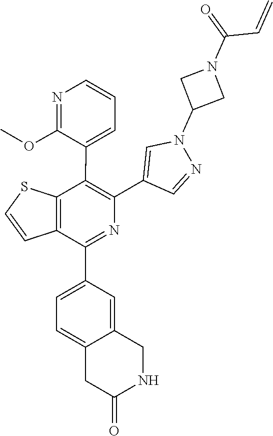 [SMILES: C=CC(=O)N1CC(n2cc(-c3nc(-c4ccc5c(c4)CNC(=O)C5)c4ccsc4c3-c3cccnc3OC)cn2)C1] | LCMS m/z [M + 1]: 563.3 ¹H NMR (400 MHz, CD₃OD) δ 8.37-8.39 (d, J = 6.8 Hz, 1H), 7.87-7.88 (m, 4H), 7.80-7.82 (d, J = 9.2 Hz, 1H), 7.73-7.74 (d, J = 5.6 Hz, 1H), 7.47-7.53 (m, 2H), 7.17-7.20 (m, 1H), 6.25-6.39 (m, 2H), 5.75- 5.78 (m, 1H), 5.23-5.28 (m, 1H), 4.71-4.75 (m, 1H), 4.64 (s, 2H), 4.57-4.58 (m, 2H), 4.46-4.51 (m, 2H), 4.29-4.32 (m, 1H), 3.78 (s, 1H), 3.73 (s, 1H). |
| 85 | 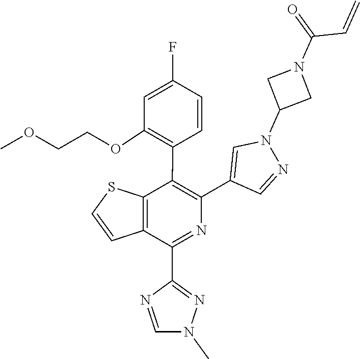 [SMILES: C=CC(=O)N1CC(n2cc(-c3nc(-c4ncn(C)n4)c4ccsc4c3-c3ccc(F)cc3OCCOC)cn2)C1] | LCMS m/z [M + 1]: 560.2 ¹H NMR (400 MHz, CD₃OD) δ 8.74 (s, 1H), 8.46 (d, J = 5.6 Hz, 1H), 8.15 (s, 1H), 7.79 (d, J = 5.6 Hz, 1H), 7.35-7.41 (m, 2H), 7.06 (dd, J = 2.3, 11.0 Hz, 1H), 6.93 (dt, J = 2.3, 8.3 Hz, 1H), 6.28-6.40 (m, 2H), 5.77 (dd, J = 2.4 Hz, 10.0 Hz, 1H), 5.20-5.30 (m, 1H), 4.70-4.75 (m, 1H), 4.45-4.60 (m, 2H), 4.28-4.35 (m, 1H), 4.15 (s, 3H), 3.90-4.10 (m, 2H), 3.38 (t, J = 4.6 Hz, 2H), 3.00 (s, 3H). |
| 86 | 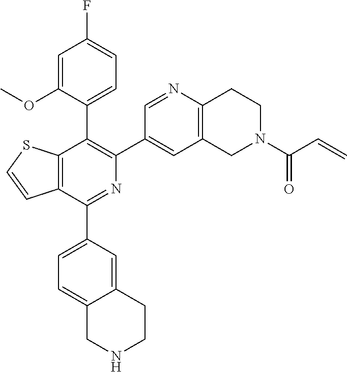 [SMILES: C=CC(=O)N1CCc2ncc(-c3nc(-c4ccc5c(c4)CCNC5)c4ccsc4c3-c3ccc(F)cc3OC)cc2C1] | LCMS m/z [M + 1]: 577.2 ¹H NMR (400 MHz, CD₃OD) δ 8.40 (s, 1H), 7.80- 7.91 (m, 4H), 7.69 (d, J = 5.6 Hz, 1H), 7.47 (d, J = 8.0 Hz, 1H), 7.37 (dd, J1 = 6.8 Hz, J2 = 8.4 Hz, 1H), 6.81-6.93 (m, 3H), 6.24-6.30 (m, 1H), 5.83 (d, J = 11.2 Hz, 1H), 4.51 (s, 2H), 3.99 (t, J = 6.0 Hz, 2H), 3.60-3.62 (m, 6H), 3.32-3.33 (m, 3H), 2.95-3.10 (m, 2H) |
| 87 | 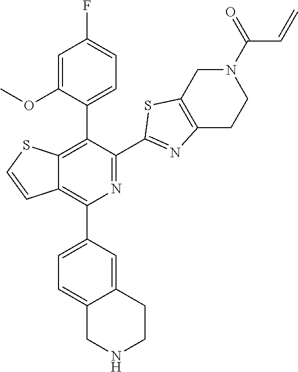 [SMILES: C=CC(=O)N1CCc2nc(-c3nc(-c4ccc5c(c4)CCNC5)c4ccsc4c3-c3ccc(F)cc3OC)sc2C1] | LCMS m/z [M + 1]: 583 ¹H NMR (400 MHz, CD₃OD) δ 7.90-7.91 (m, 2H), 7.84 (d, J = 5.6 Hz, 1H), 7.73 (d, J = 5.6 Hz, 1H), 7.48 (d, J = 7.6 Hz, 1H), 7.34 (dd, J = = 8.4, 6.8 Hz, 1H), 6.84-6.95 (m, 3H), 6.24-6.28 (m, 1H), 5.79-5.83 (m, 1H), 4.52 (s, 2H), 3.93 (t, J = 5.2 Hz, 2H), 3.58-3.66 (m, 6H), 2.70-2.78 (m, 2H). |
| 88 | 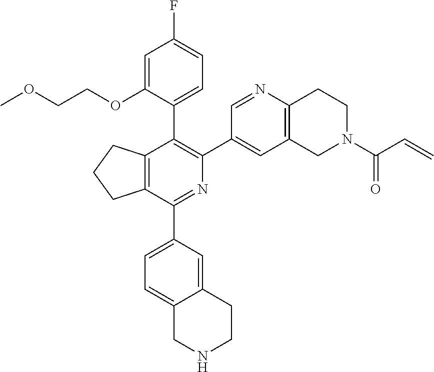 [SMILES: C=CC(=O)N1CCc2ncc(-c3nc(-c4ccc5c(c4)CCNC5)c4c(c3-c3ccc(F)cc3OCCOC)CCC4)cc2C1] | LCMS m/z [M + 1]: 605.3 ¹H NMR (400 MHz, CD₃OD) δ 8.36 (s, 1H), 7.94- 8.01 (m, 1H), 7.75-7.78 (m, 2H) 7.39 (d, J = 8.0 Hz, 1H), 7.10 (dd, J = 8.4, 6.7 Hz, 1H), 6.70-6.89 (m, 3H), 6.20-6.31 (m, 1H), 5.81 (d, J = 10.8 Hz, 1H), 4.46 (s, 2H), 4.05-4.11 (m, 1H), 3.88-4.00 (m, 3H), 3.55-3.59 (m, 4H), 3.27 (s, 3H), 3.20-3.27 (m, 4H), 2.95-3.14 (m, 2H), 2.82-2.90 (m, 2H), 2.11-2.20 (m, 2H). |
| 89 | 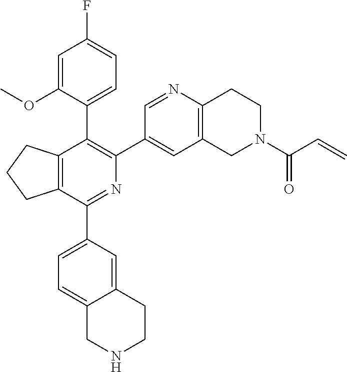 [SMILES: C=CC(=O)N1CCc2ncc(-c3nc(-c4ccc5c(c4)CCNC5)c4c(c3-c3ccc(F)cc3OC)CCC4)cc2C1] | LCMS m/z [M + 1]: 561.3 ¹H NMR (400 MHz, CD₃OD) δ 8.34 (s, 1H), 7.95- 8.01 (m, 1H), 7.75-7.78 (m, 2H) 7.39 (d, J = 8.0 Hz, 1H), 7.10 (dd, J = 8.4, 6.7 Hz, 1H), 6.70-6.89 (m, 3H), 6.20-6.31 (m, 1H), 5.81 (d, J = 10.2 Hz, 1H), 4.46 (s, 2H), 3.98 (t, J = 6.0 Hz, 2H), 3.66 (s, 3H), 3.57 (t, J = 6.4 Hz, 2H), 3.18-3.27 (m, 4H), 3.00-3.12 (m, 2H), 2.73-2.92 (m, 2H), 2.12-2.16 (m, 2H). |
| 90 | 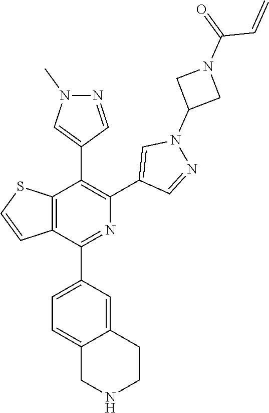 [SMILES: C=CC(=O)N1CC(n2cc(-c3nc(-c4ccc5c(c4)CCNC5)c4ccsc4c3-c3cnn(C)c3)cn2)C1] | LCMS m/z [M + 1]: 522.2 ¹H NMR (400 MHz, CD₃OD) δ 7.92 (s, 2H), 7.83- 7.87 (m, 3H), 7.70 (s, 1H), 7.67 (d, J = 5.6 Hz, 1H), 7.60 (s, 1H), 7.50 (d, J = 8.0 Hz, 1H), 6.37-6.44 (m, 1H), 6.29-6.33 (m, 1H), 5.80 (dd, J = 10.0, 2.0 Hz, 1H), 5.29-5.36 (m, 1H), 4.79 (t, J = 8.8 Hz, 1H), 4.63- 4.67 (m, 1H), 4.53-4.57 (m, 3H), 4.37-4.41 (m, 1H), 4.05 (s, 3H), 3.63 (t, J = 6.0 Hz, 2H), 3.30-3.33 (m, 2H). |
| 91 | 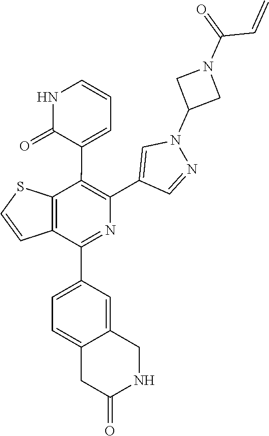 [SMILES: C=CC(=O)N1CC(n2cc(-c3nc(-c4ccc5c(c4)CNC(=O)C5)c4ccsc4c3-c3ccc[nH]c3=O)cn2)C1] | LCMS m/z [M + 1]: 549.2 ¹H NMR (400 MHz, CD₃OD) δ 8.06-8.07 (d, J = 3.6 Hz, 1H), 7.82-7.86 (m, 4H), 7.67-7.72 (m, 3H), 7.49-7.51 (d, J = 8.4 Hz, 1H), 6.59-6.63 (d, J = 6.8 Hz, 1H), 6.25-6.40 (m, 2H), 5.76-5.79 (m, 1H), 5.28- 5.33 (m, 1H), 4.74-4.76 (m, 1H), 4.63 (s, 3H), 4.49- 4.53 (m, 1H), 4.34-4.37 (m, 1H), 3.72 (s, 2H). |
| 92 | 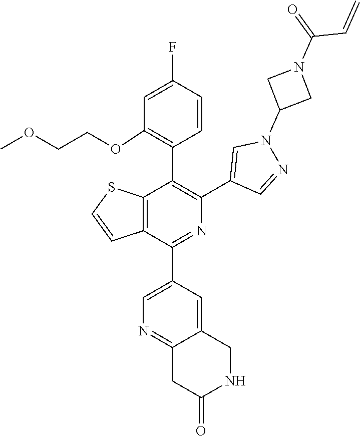 | LCMS m/z [M + 1]: 653.2 ¹H NMR (400 MHz, CD₃OD) δ 9.06 (s, 1H), 8.41 (s, 1H), 7.76 (d, J = 5.2 Hz, 2H), 7.71 (d, J = 5.6 Hz, 1H), 7.49 (d, J = 5.2 Hz, 1H), 7.33 (dd, J = 8.4, 6.8 Hz, 1H), 7.07 (dd, J = 10.8, 2.0 Hz, 1H), 6.91-6.95 (m, 1H), 6.39-6.29 (m, 2H), 5.77 (dd, J = 10.0, 2.0 Hz, 1H), 5.24 (s, 1H), 4.73 (d, J = 12.8 Hz, 3H), 4.58- 4.48 (m, 2H), 4.30 (dd, J = 10.8, 4.8 Hz, 1H), 3.98- 4.07 (m, 2H), 3.40 (t, J = 4.4 Hz, 2H), 3.03 (s, 3H). |
| 93 | 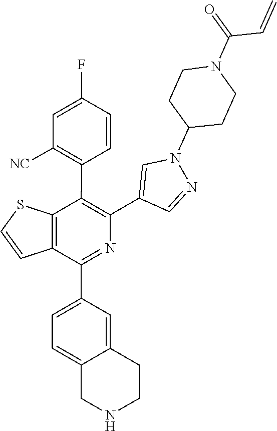 [SMILES: C=CC(=O)N1CCC(n2cc(-c3nc(-c4ccc5c(c4)CCNC5)c4ccsc4c3-c3ccc(F)cc3C#N)cn2)CC1] | LCMS m/z [M + 1]: 589 ¹H NMR (400 MHz, CD₃OD) δ 7.82-7.89 (m, 3H), 7.68-7.78 (m, 5H), 7.46 (d, J = 8 Hz, 1H), 7.17 (s, 1H), 6.75-6.82 (m, 1H), 6.20 (dd, J = 16.8, 1.6 Hz, 1H), 5.73-5.76 (m, 1H), 4.55-4.70 (m, 1H), 4.35-4.40 (m, 3H), 4.15-4.25 (m, 1H), 3.60 (t, J = 6.4 Hz, 2H), 3.23-3.30 (m, 1H), 2.82-2.95 (m, 1H), 2.05-2.15 (m, 2H), 1.80-1.98 (m, 2H). |
| 94 | 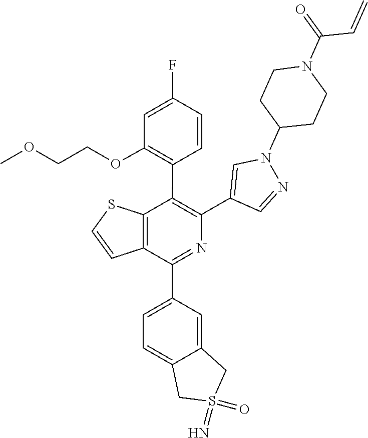 [SMILES: C=CC(=O)N1CCC(n2cc(-c3nc(-c4ccc5c(c4)CS(=N)(=O)C5)c4ccsc4c3-c3ccc(F)cc3OCCOC)cn2)CC1] | LCMS m/z [M + 1]: 672.2 ¹H NMR (400 MHz, CD₃OD) δ 7.90-7.94 (m, 2H), 7.68 (s, 1H), 7.57-7.63 (m, 3H), 7.29-7.34 (m, 2H), 7.05 (dd, J = 11.2, 2.2 Hz, 1H), 6.91 (dt, J = 8.3, 2.3 Hz, 1H), 6.79 (dd, J = 16.8, 11.2 Hz, 1H), 6.20 (dd, J = 16.8, 2.2 Hz, 1H), 5.75 (dd, J = 10.7, 2.2 Hz, 1H), 4.56-4.65 (m, 6H), 4.37-4.43 (m, 1H), 4.19-4.22 (m, 1H) ,4.02-4.06 (m, 1H), 3.93-3.96 (m, 1H), 3.39 (t, J = 4.6 Hz, 2H), 3.02 (s, 3H), 2.87-2.96 (m, 1H), 2.08- 2.09 (m, 2H), 1.84-1.90 (m, 2H). |
| 95 | 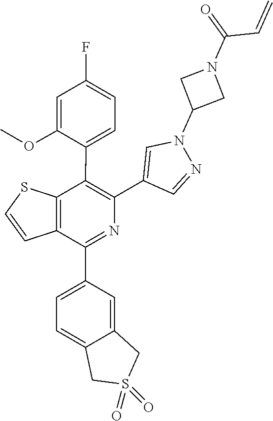 [SMILES: C=CC(=O)N1CC(n2cc(-c3nc(-c4ccc5c(c4)CS(=O)(=O)C5)c4ccsc4c3-c3ccc(F)cc3OC)cn2)C1] | LCMS m/z [M + 1]: 601.2 ¹H NMR (400 MHz, CD₃OD) δ 7.97-8.01 (m, 2H), 7.85-7.90 (m, 2H), 7.68-7.73 (m, 2H), 7.52 (s, 1H), 7.36 (t, J = 7.2 Hz, 1H), 7.05 (d, J = 10.0 Hz, 1H), 6.93 (t, J = 7.0 Hz, 1H), 6.25-6.39 (m, 2H), 5.77(d, J = 9.0 Hz, 1H), 5.21-5.29 (m, 1H), 4.59-4.60 (m, 6H), 4.47-4.52 (m, 1H), 4.27-4.35 (m, 1H), 3.67 (s, 3H). |
| 96 | 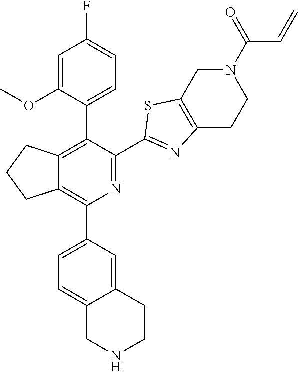 [SMILES: C=CC(=O)N1CCc2nc(-c3nc(-c4ccc5c(c4)CCNC5)c4c(c3-c3ccc(F)cc3OC)CCC4)sc2C1] | LCMS m/z [M + 1]: 567.2 ¹H NMR (400 MHz, CD₃OD) δ 7.82 (s, 1H), 7.80 (s, 1H), 7.40 (d, J = 8.8 Hz, 1H), 7.14 (t, J = 6.8 Hz, 1H), 6.76-6.88 (m, 3H), 6.20-6.26 (m, 1H), 5.78-5.80 (m, 1H), 4.82 (s, 2H), 4.46 (s, 2H), 3.90-3.92 (m, 2H), 3.64 (s, 3H), 3.57 (t, J = 6.4 Hz, 2H), 3.22-3.25 (m, 2H), 2.72-2.85 (m, 4H), 2.11-2.13 (m, 2H). |
| 97 | 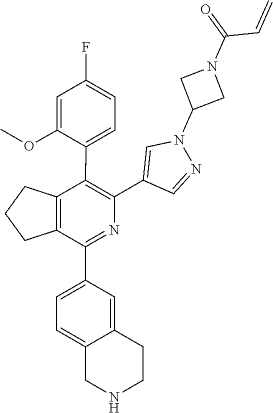 [SMILES: C=CC(=O)N1CC(n2cc(-c3nc(-c4ccc5c(c4)CCNC5)c4c(c3-c3ccc(F)cc3OC)CCC4)cn2)C1] | LCMS m/z [M + 1]: 550.3 ¹H NMR (400 MHz, CD₃OD) δ 7.75-7.80 (m, 3H), 7.46-7.51 (m, 2H), 7.16 (d, J = 6.4 Hz, 1H), 6.97 (dd, J = 10.8, 2.2 Hz, 1H), 6.84 (dt, J = 8.3, 2.4 Hz, 1H), 6.24-6.38 (m, 2H), 5.77 (dd, J = 9.8, 2.4 Hz, 1H), 5.21-5.23 (m, 1H), 4.70-4.75 (m, 2H), 4.45-4.55 (m, 3H), 4.27 (dd, J = 11.0, 5.3 Hz, 1H), 3.70 (s, 3H), 3.58 (t, J = 6.4 Hz, 2H), 3.17-3.28 (m, 4H), 2.83-2.91 (m, 2H), 2.13-2.20 (m, 2H). |
| 98 | 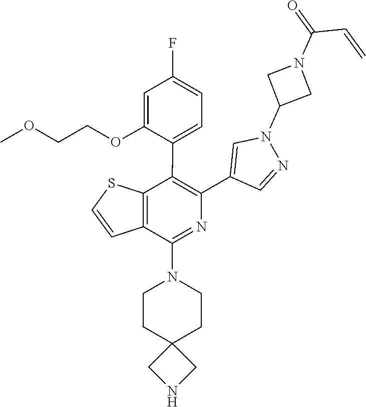 [SMILES: C=CC(=O)N1CC(n2cc(-c3nc(N4CCC5(CC4)CNC5)c4ccsc4c3-c3ccc(F)cc3OCCOC)cn2)C1] | LCMS m/z [M + 1]: 603.3 ¹H NMR (400 MHz, CD₃OD) δ 7.48-7.61 (m, 4H), 7.21 (dd, J = 8.3, 6.7 Hz, 1H), 7.01 (dd, J = 11.1, 2.3 Hz, 1H), 6.83-6.89 (m, 1H), 6.24-6.40 (m, 2H), 5.77 (dd, J = 9.9, 2.4 Hz, 1H), 5.16-5.24 (m, 1H), 4.72 (t, J = 8.9 Hz, 1H), 4.44-4.57 (m, 2H), 4.28 (dd, J = 10.7, 5.3 Hz, 1H), 3.94-4.07 (m, 5H), 3.62-3.67 (m, 3H), 3.40 (t, J = 4.6 Hz, 2H), 3.03 (s, 3H), 2.09-2.18 (m, 4H). |
| 99 | 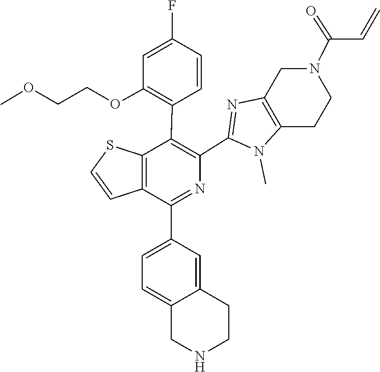 [SMILES: C=CC(=O)N1CCc2c(nc(-c3nc(-c4ccc5c(c4)CCNC5)c4ccsc4c3-c3ccc(F)cc3OCCOC)n2C)C1] | LCMS m/z [M + 1]: 624.3 ¹H NMR (400 MHz, CD₃OD) δ 8.07 (d, J = 5.6 Hz, 1H), 7.83-7.88 (m, 3H), 7.54-7.55 (m, 1H), 7.47 (d, J = 8.0 Hz, 1H), 7.03 (d, J = 10.8 Hz, 1H), 6.7-6.98 (m, 2H), 6.28 (d, J = 16.8 Hz, 1H), 5.84 (d, J = 10.4 Hz, 1H), 4.71 (s, 2H), 4.50 (s, 2H), 4.00-4.09 (m, 4H), 3.54-3.64 (m, 5H), 3.47 (t, J = 4.4 Hz, 2H), 3.25-3.30 (m, 2H), 3.16 (s, 3H), 2.75-2.90 (m, 2H). |
| 100 | 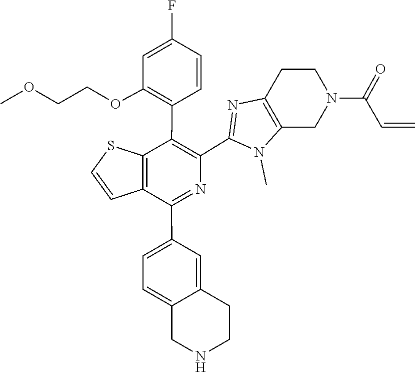 [SMILES: C=CC(=O)N1CCc2nc(-c3nc(-c4ccc5c(c4)CCNC5)c4ccsc4c3-c3ccc(F)cc3OCCOC)n(C)c2C1] | LCMS m/z [M + 1]: 624.3 ¹H NMR (400 MHz, CD₃OD) δ 8.07 (d, J = 5.6 Hz, 1H), 7.84-7.89 (m, 3H), 7.56 (t, J = 8.0 Hz, 1H), 7.47 (d, J = 8.0 Hz, 1H), 7.04 (dd, J = 11.0, 2.2 Hz, 1H), 6.93 (dt, J = 8.3, 2.2 Hz, 1H), 6.83-6.90 (m, 1H), 6.29 (dd, J = 16.7, 1.6 Hz, 1H), 5.84 (dd, J = 10.6, 1.6 Hz, 1H), 4.69-4.80 (m, 2H), 4.51 (s, 2H), 3.88-4.13 (m, 4H), 3.64-3.72 (m, 3H), 3.60 (t, J = 6.4 Hz, 2H), 3.48 (t, J = 4.6 Hz, 2H), 3.27 (t, J = 6.4 Hz, 2H), 3.17 (s, 3H), 2.72-2.85 (m, 2H). |
| 101 | 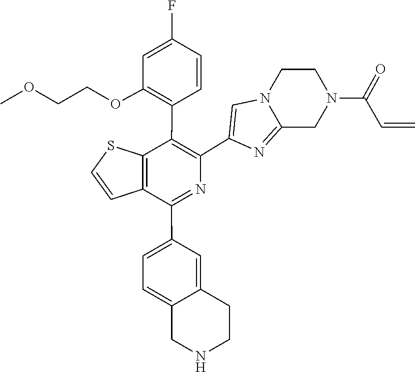 [SMILES: C=CC(=O)N1CCn2cc(-c3nc(-c4ccc5c(c4)CCNC5)c4ccsc4c3-c3ccc(F)cc3OCCOC)nc2C1] | LCMS m/z [M + 1]: 610.3 ¹H NMR (400 MHz, CD₃OD) δ 7.91-7.97 (m, 2H), 7.87 (d, J = 5.6 Hz, 1H), 7.74 (d, J = 5.6 Hz, 1H), 7.49 (d, J = 8.0 Hz, 1H), 7.39-7.45 (m, 1H), 7.14 (dd, J = 11.0, 2.3 Hz, 1H), 6.97-7.05 (m, 1H), 6.73-6.91 (m, 1H), 6.52 (s, 1H), 6.29-6.38 (m, 1H), 5.88 (dd, J = 10.6, 1.7 Hz, 1H), 5.02-5.21 (m, 2H), 4.52 (s, 2H), 4.04-4.22 (m, 6H), 3.62 (t, J = 6.3 Hz, 2H), 3.38-3.45 (m, 2H), 3.03 (s, 3H). |
| 102 | 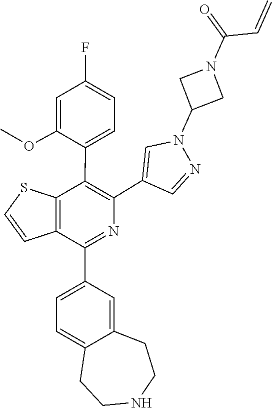 [SMILES: C=CC(=O)N1CC(n2cc(-c3nc(-c4ccc5c(c4)CCNCC5)c4ccsc4c3-c3ccc(F)cc3OC)cn2)C1] | LCMS m/z [M + 1]: 580.3 ¹H NMR (400 MHz, CD₃OD) δ 7.70-7.86 (m, 4H), 7.66 (d, J = 5.6 Hz, 1H), 7.47-7.54 (m, 2H), 7.28-7.36 (m, 1H), 7.04 (dd, J = 11.0, 2.1 Hz, 1H), 6.87-6.95 (m, 1H), 6.22-6.42 (m, 2H), 5.77 (dd, J = 10.0, 2.1 Hz, 1H), 5.18-5.29 (m, 1H), 4.73 (t, J = 8.4 Hz, 1H), 4.43-4.59 (m, 2H), 4.24-4.34 (m, 1H), 3.67 (s, 3H), 3.36-3.44 (m, 4H). |
| 103 | 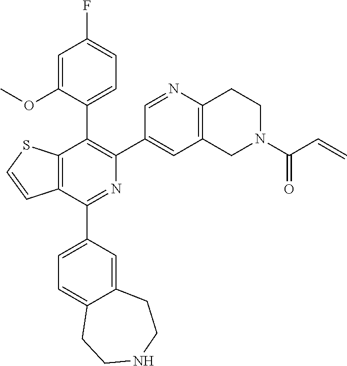 [SMILES: C=CC(=O)N1CCc2ncc(-c3nc(-c4ccc5c(c4)CCNCC5)c4ccsc4c3-c3ccc(F)cc3OC)cc2C1] | LCMS m/z [M + 1]: 591.2 ¹H NMR (400 MHz, CD₃OD) δ 8.44 (s, 1H), 8.10 (s, 1H), 7.81-7.86 (m, 3H), 7.72 (d, J = 5.6 Hz, 1H), 7.49 (d, J = 8.0 Hz, 1H), 7.40 (dd, J = 8.4, 6.4 Hz, 1H), 6.93 (dd, J = 11.2, 2.4 Hz, 1H), 6.82-6.87 (m, 2H), 6.27 (d, J = 17.2 Hz, 1H), 5.82 (d, J = 10.4 Hz, 1H), 4.00 (t, J = 6.0 Hz, 2H), 3.62 (s, 3H), 3.35-3.44 (m, 4H), 3.11-3.13 (m, 2H). |
| 104 | 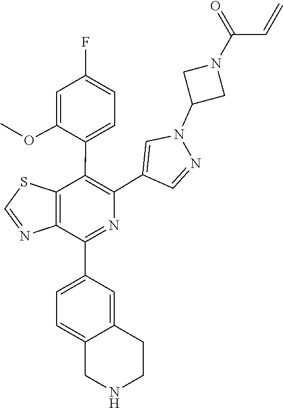 [SMILES: C=CC(=O)N1CC(n2cc(-c3nc(-c4ccc5c(c4)CCNC5)c4ncsc4c3-c3ccc(F)cc3OC)cn2)C1] | LCMS m/z [M + 1]: 567.2 ¹H NMR (400 MHz, CD₃OD) δ 9.27 (s, 1H), 8.51- 8.53 (m, 2H), 7.74 (s, 1H), 7.55(s, 1H), 7.48 (d, J = 8.0 Hz, 1H), 7.32-7.36 (m, 1H), 7.09 (dd, J = 11.2, 2.2 Hz, 1H), 6.92-6.97 (m, 1H), 6.28-6.43 (m, 2H), 5.80 (dd, J = 9.6, 2.0 Hz, 1H), 5.24-5.31 (m, 1H), 4.76 (t, J = 8.8 Hz, 1H), 4.58-4.62 (m, 1H), 4.50-4.54 (m, 3H), 4.34-4.36 (m, 1H), 3.72 (s, 3H), 3.63 (t, J = 6.4 Hz, 2H), 3.27-3.29 (m, 2H). |
| 105 | 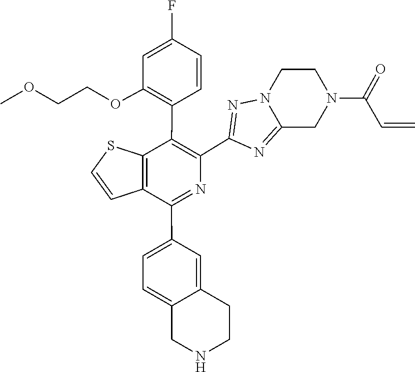 [SMILES: C=CC(=O)N1CCn2nc(-c3nc(-c4ccc5c(c4)CCNC5)c4ccsc4c3-c3ccc(F)cc3OCCOC)nc2C1] | LCMS m/z [M + 1]: 611.2 ¹H NMR (400 MHz, CD₃OD) δ 7.92 (s, 1H), 7.90 (d, J = 8.0 Hz, 1H), 7.85 (d, J = 5.6 Hz, 1H), 7.70 (d, J = 5.6 Hz, 1H), 7.45 (d, J = 8.0 Hz, 1H), 7.32-7.35 (m, 1H), 6.75-6.92 (m, 3H), 6.30 (d, J = 16.8 Hz, 1H), 5.84 (dd, J = 10.6, 1.7 Hz, 1H), 4.90-5.05 (m, 2H), 4.50 (s, 2H), 4.09-4.20 (m, 4H), 3.96-4.04 (m, 1H), 3.82-3.89 (m, 1H), 3.60 (t, J = 6.4 Hz, 2H), 3.41 (t, J = 4.8 Hz, 2H), 3.20-3.31 (m, 2H), 3.06 (s, 3H). |
| 106 | 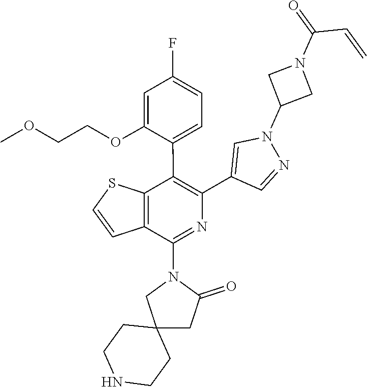 [SMILES: C=CC(=O)N1CC(n2cc(-c3nc(N4CC5(CCNCC5)CC4=O)c4ccsc4c3-c3ccc(F)cc3OCCOC)cn2)C1] | LCMS m/z [M + 1]: 631.3 ¹H NMR (400 MHz, CD₃OD) δ 7.57 (d, J = 6.4 Hz, 1H), 7.55 (d, J = 5.08 Hz, 1H), 7.50(d, J = 4.8 Hz, 1H), 7.43 (d, J = 4 Hz, 1H), 7.26 (dd, J = 8.4, 7.1 Hz, 1H), 7.05 (dd, J = 11.2, 2.3 Hz, 1H), 6.91 (dt, J = 8.3, 2.4 Hz, 1H), 6.25-6.39 (m, 2H), 5.77 (dd, J = 9.9, 2.4 Hz, 1H), 5.17-5.21 (m, 1H),4.72 (t, J = 8.8 Hz, 1H), 4.45-4.55 (m, 2H), 4.26-4.30 (m, 1H), 4.18 (s, 2H), 3.95-4.06 (m, 2H), 3.31-3.38(m, 4H), 3.01 (s, 3H), 2.78 (s, 2H), 2.09 (t, J = 5.6 Hz, 4H). |
| 107 | 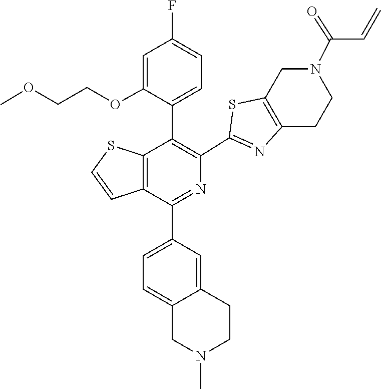 [SMILES: C=CC(=O)N1CCc2nc(-c3nc(-c4ccc5c(c4)CCN(C)C5)c4ccsc4c3-c3ccc(F)cc3OCCOC)sc2C1] | LCMS m/z [M + 1]: 641.3 ¹H NMR (400 MHz, CD₃OD) δ 7.89-7.97 (m, 2H), 7.82 (d, J = 5.5 Hz, 1H), 7.72 (d, J = 5.6 Hz, 1H), 7.44 (d, J = 8.4 Hz, 1H), 7.36 (t, J = 8.0 Hz, 1H), 6.81-6.95 (m, 3H), 6.24 (d, J = 16.7 Hz, 1H), 5.79 (d, J = 9.8 Hz, 1H), 4.49-4.77 (m, 2H), 3.98-4.05 (m, 1H), 3.80-3.97 (m, 4H), 3.31-3.62 (m, 5H), 3.11 (s, 3H), 3.00 (s, 3H), 2.70-2.73 (m, 2H). |
| 108 | 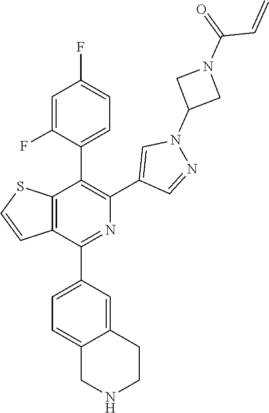 [SMILES: C=CC(=O)N1CC(n2cc(-c3nc(-c4ccc5c(c4)CCNC5)c4ccsc4c3-c3ccc(F)cc3F)cn2)C1] | LCMS m/z [M + 1]: 554.2 ¹H NMR (400 MHz, CD₃OD) δ 7.87-7.92 (m, 2H), 7.72-7.75 (m, 2H), 7.66-7.68 (m, 1H), 7.53-7.60 (m, 1H), 7.45-7.51 (m, 2H), 7.22-7.28 (m, 2H), 6.27-6.42 (m, 2H), 5.79 (dd, J = 9.9, 2.2 Hz, 1H), 5.21-5.30 (m, 1H), 4.75 (t, J = 8.8 Hz, 1H), 4.58-4.65 (m, 1H), 4.46- 4.55 (m, 3H), 4.31-4.37 (m, 1H), 3.63 (t, J = 6.4 Hz, 2H). |
| 109 | 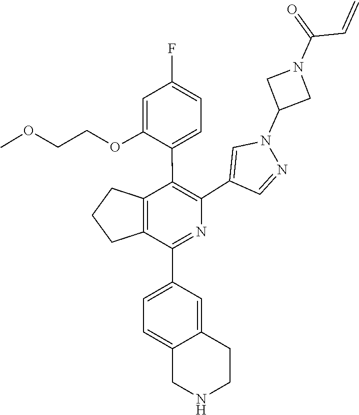 [SMILES: C=CC(=O)N1CC(n2cc(-c3nc(-c4ccc5c(c4)CCNC5)c4c(c3-c3ccc(F)cc3OCCOC)CCC4)cn2)C1] | LCMS m/z [M + 1]: 594.3 ¹H NMR (400 MHz, CD₃OD) δ 7.73-7.77 (m, 3H), 7.50 (d, J = 5.4 Hz, 1H), 7.44 (d, J = 8.4 Hz, 1H), 7.13 (t, J = 8.2 Hz, 1H), 6.99 (dd, J = 11.0, 2.2 Hz, 1H), 6.82 (dt, J = 8.3, 2.3 Hz, 1H), 6.24-6.39 (m, 2H), 5.76 (dd, J = 9.8, 2.3 Hz, 1H), 5.18-5.25 (m, 1H), 4.72 (t, J = 8.7 Hz, 1H), 4.45-4.54 (m, 4H), 4.25-4.29 (m, 1H), 4.09-4.14 (m, 1H), 3.98-4.03 (m, 1H), 3.52-3.60 (m, 5H), 3.16-3.27 (m, 9H), 2.79-2.92 (m, 2H), 2.10- 2.18 (m, 2H). |
| 110 | 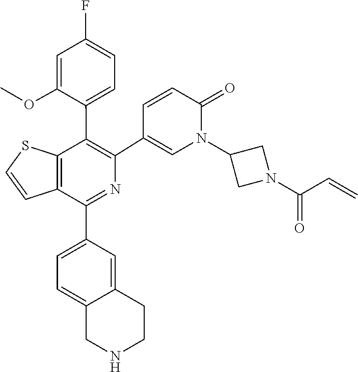 [SMILES: C=CC(=O)N1CC(n2cc(-c3nc(-c4ccc5c(c4)CCNC5)c4ccsc4c3-c3ccc(F)cc3OC)ccc2=O)C1] | LCMS m/z [M + 1]: 593.2 ¹H NMR (400 MHz, CD₃OD) δ 8.19 (s, 1H), 7.84- 7.89 (m, 3H), 7.79-7.81 (m, 1H), 7.70 (d, J = 5.6 Hz, 1H), 7.49 (d, J = 8.0 Hz, 1H), 7.32-7.37 (m, 1H), 6.90 (dd, J = 10.9, 1.8 Hz, 1H), 6.80-6.83 (m, 2H), 6.22- 6.37 (m, 2H), 5.74 (dd, J = 10.1, 1.6 Hz, 1H), 5.38- 5.42 (m, 1H), 4.69 (t, J = 9.8 Hz, 1H), 4.51 (s, 2H), 4.42-4.47 (m, 1H), 4.25-4.27 (m, 1H), 3.98-4.02 (m, 1H), 3.58-3.61 (m, 5H), 3.28 (t, J = 6.5 Hz, 2H). |
| 111 | 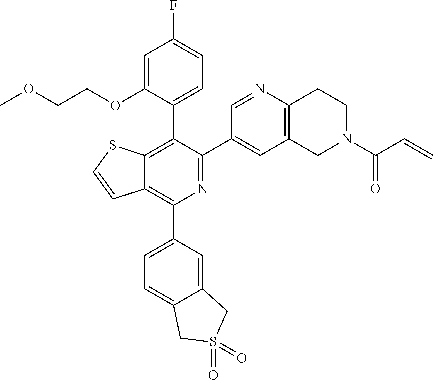 [SMILES: C=CC(=O)N1CCc2ncc(-c3nc(-c4ccc5c(c4)CS(=O)(=O)C5)c4ccsc4c3-c3ccc(F)cc3OCCOC)cc2C1] | LCMS m/z [M + 1]: 656.2 ¹H NMR (400 MHz, CD₃OD) δ 8.60 (s, 1H), 8.40 (s, 1H), 7.97-8.00 (m, 2H), 7.91 (d, J = 5.6 Hz, 1H), 7.78 (d, J = 5.6 Hz, 1H), 7.63 (d, J = 8.0 Hz, 1H), 7.47- 7.51 (m, 1H), 6.70-6.98 (m, 3H), 6.22-6.35 (m, 1H), 5.83 (d, J = 10.8 Hz, 1H), 4.57 (s, 2H), 4.58 (s, 2H), 3.98-4.05 (m, 3H), 3.88-3.98 (m, 1H), 3.44 (t, J = 3.6 Hz, 2H), 3.30-3.31 (m, 2H), 3.10-3.25 (m, 5H). |
| 112 | 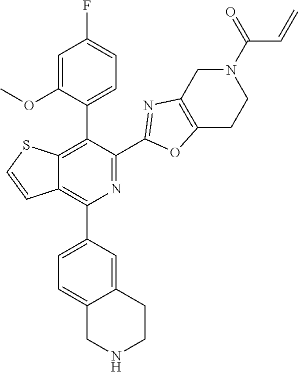 [SMILES: C=CC(=O)N1CCc2oc(-c3nc(-c4ccc5c(c4)CCNC5)c4ccsc4c3-c3ccc(F)cc3OC)nc2C1] | LCMS m/z [M + 1]: 567.2 ¹H NMR (400 MHz, CD₃OD) δ 7.88-7.89 (m, 3H), 7.71 (d, J = 5.6 Hz, 1H), 7.37-7.48 (m, 2H), 6.91-6.95 (m, 1H), 6.74-6.88 (m, 2H), 6.23 (d, J = 16.4 Hz, 1H), 5.79 (d, J = 10.8 Hz, 1H), 4.57 (s, 2H), 4.49 (s, 2H), 3.93-3.98 (m, 2H), 3.58-3.61 (m, 5H), 2.70-2.73 (m, 2H). |
| 113 | 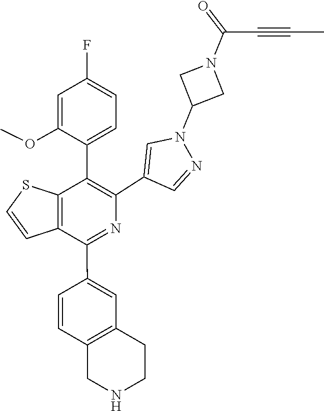 [SMILES: CC#CC(=O)N1CC(n2cc(-c3nc(-c4ccc5c(c4)CCNC5)c4ccsc4c3-c3ccc(F)cc3OC)cn2)C1] | LCMS m/z [M + 1]: 578.2 ¹H NMR (400 MHz, CD₃OD) δ 7.87-7.89 (m, 2H), 7.76-7.78 (m, 1H), 7.71-7.73 (m, 1H), 7.66 (d, J = 5.6 Hz, 1H), 7.52 (s, 1H), 7.50 (d, J = 8.0 Hz, 1H), 7.33 (t, J = 7.6 Hz, 1H), 7.06 (dd, J = 11.2, 2.4 Hz, 1H), 6.91-6.96 (m, 1H), 5.19-5.26 (m, 1H), 4.66 (t, J = 8.8 Hz, 1H), 4.42-4.53 (m, 4H), 4.22-4.26 (m, 1H), 3.68 (s, 3H), 3.62 (t, J = 6.4 Hz, 2H), 3.30-3.33 (m, 2H), 2.04 (s, 3H). |
| 114 | 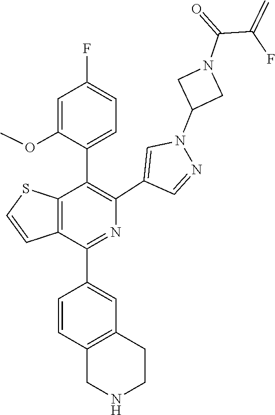 [SMILES: C=C(F)C(=O)N1CC(n2cc(-c3nc(-c4ccc5c(c4)CCNC5)c4ccsc4c3-c3ccc(F)cc3OC)cn2)C1] | LCMS m/z [M + 1]: 584.2 ¹H NMR (400 MHz, CD₃OD) δ 7.87-7.89 (m, 2H), 7.80-7.82 (m, 1H), 7.75-7.77 (m, 1H), 7.66 (d, J = 5.6 Hz, 1H), 7.53 (s, 1H), 7.51 (d, J = 8.0 Hz, 1H), 7.34 (t, J = 6.8 Hz, 1H), 7.06 (dd, J = 11.2, 2.4 Hz, 1H), 6.91-6.95 (m, 1H), 5.65 (d, J = 3.6 Hz, 1H), 5.53 (d, J = 3.2 Hz, 1H), 5.22-5.29 (m, 1H), 4.67-4.70 (m, 1H), 4.51-4.55 (m, 3H), 4.32-4.34 (m, 1H), 3.68 (s, 3H), 3.62 (t, J = 6.4 Hz, 2H), 3.29-3.32 (m, 3H). |
| 115 | 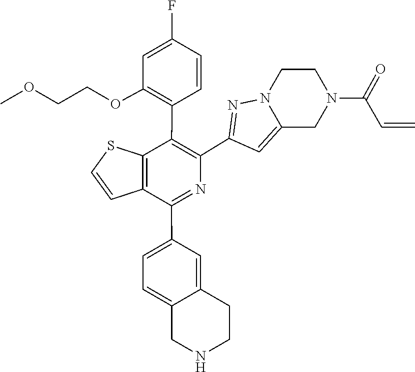 [SMILES: C=CC(=O)N1CCn2nc(-c3nc(-c4ccc5c(c4)CCNC5)c4ccsc4c3-c3ccc(F)cc3OCCOC)cc2C1] | LCMS m/z [M + 1]: 610.2 ¹H NMR (400 MHz, CD₃OD) δ 7.87-7.92 (m, 2H), 7.82 (d, J = 5.6 Hz, 1H), 7.67 (d, J = 5.6 Hz, 1H), 7.47 (d, J = 8.0 Hz, 1H), 7.31 (dd, J = 8.0, 6.8 Hz, 1H), 6.99 (dd, J = 11.0, 2.4 Hz, 1H), 6.75-6.94 (m, 2H), 6.25-6.32 (m, 1H), 5.78-5.95 (m, 2H), 4.50 (s, 2H), 4.18-4.26 (m, 2H), 4.02-4.15 (m, 3H), 3.90-3.97 (m, 1H), 3.60 (t, J = 6.4 Hz, 2H), 3.41 (t, J = 4.8 Hz, 2H), 3.25-3.29 (m, 2H), 3.07 (s, 3H). |
| 116 | 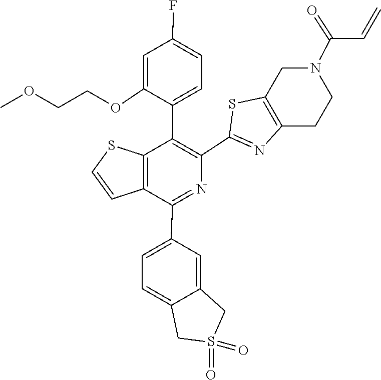 [SMILES: C=CC(=O)N1CCc2nc(-c3nc(-c4ccc5c(c4)CS(=O)(=O)C5)c4ccsc4c3-c3ccc(F)cc3OCCOC)sc2C1] | LCMS m/z [M + 1]: 662.1 ¹HNMR (400 MHz, CD₃OD) δ 7.95- 8.05 (m, 2H), 7.84 (d, J = 5.6 Hz, 1H), 7.74 ( d, J = 5.6 Hz, 1H), 7.61 (d, J = 8.0 Hz, 1H), 7.37 (t, J = 8 Hz, 1H), 6.96 (d, J = 1.2 Hz, 1H), 6.84-6.89 (m, 2H), 6.24 (d, J = 16.8 Hz, 1H), 5.79 (d, J = 11.2 Hz, 1H), 4.60 (s, 2H), 4.58 (s, 2H), 4.01-4.06 (m, 1H), 3.86- 3.95 (m, 3H), 3.00 (s, 3H), 2.65-2.74 (m, 2H). |
| 117 | 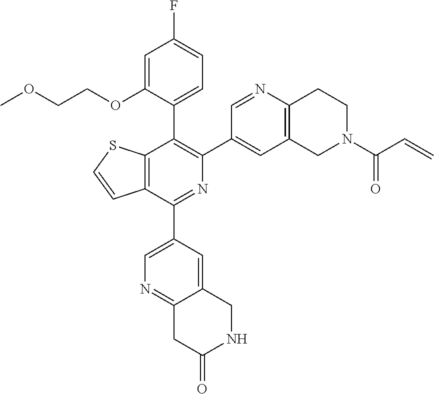 [SMILES: C=CC(=O)N1CCc2ncc(-c3nc(-c4cnc5c(c4)CNC(=O)C5)c4ccsc4c3-c3ccc(F)cc3OCCOC)cc2C1] | LCMS m/z [M + 1]: 636 ¹H NMR (400 MHz, CD₃OD) δ 9.05 (s, 1H), 8.56 (s, 1H), 8.38 (s, 1H), 8.28 (s, 1H), 7.90-7.90 (d, J = 5.6 Hz, 1H), 7.80 (d, J = 5.6 Hz, 1H), 7.46 (dd, J = 8.4, 6.4 Hz, 1H), 6.94-6.98 (m, 1H), 6.84-6.91 (m, 2H), 6.27 (d, J = 16.8 Hz, 1H), 5.82 (d, J = 10.4 Hz, 1H), 4.70 (s, 2H), 3.98-4.05 (m, 3H), 3.88-3.91 (m, 1H), 3.43 (t, J = 3.6 Hz, 2H), 3.15 (s, 6H). |
| 118 | 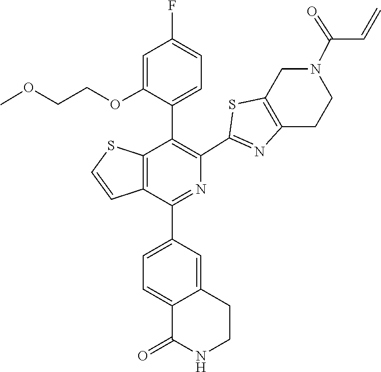 [SMILES: C=CC(=O)N1CCc2nc(-c3nc(-c4ccc5c(c4)CCNC5=O)c4ccsc4c3-c3ccc(F)cc3OCCOC)sc2C1] | LCMS m/z [M + 1]: 641.2 ¹H NMR (400 MHz, CD₃OD) δ 8.15 (d, J = 8.0 Hz, 1H), 7.95 (d, J = 8.0 Hz, 1H), 7.91 (s, 1H), 7.86 (d, J = 5.5 Hz, 1H), 7.75 (d, J = 5.5 Hz, 1H), 7.39 (dd, J = 8.2, 6,8 Hz, 1H), 6,96 (d, J = 9.4 Hz, 1H), 6.73-6,90 (m, 2H), 6.24 (d, J = 16.2 Hz, 1H), 5.79 (d, J = 10.5 Hz, 1H), 4.00-4.08 (m, 1H), 3.83-3.96 (m, 3H), 3.61 (t, J = 6.6 Hz, 2H),3.3-3.38 (m, 4H), 3.15 (t, J = 6.5 Hz, 2H), 3.00 (s, 3H), 2.65-2.80 (m, 2H). |
| 119 | 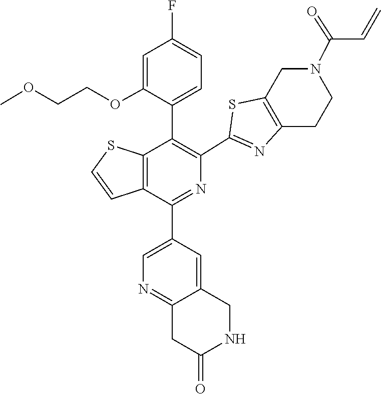 [SMILES: C=CC(=O)N1CCc2nc(-c3nc(-c4cnc5c(c4)CNC(=O)C5)c4ccsc4c3-c3ccc(F)cc3OCCOC)sc2C1] | LCMS m/z [M + 1]: 642.2 ¹H NMR (400 MHz, CD₃OD) δ 9.13 (s, 1H), 8.46- 8.57 (m, 1H), 7.90 (d, J = 5.6 Hz, 1H), 7.80 (d, J = 5.6 Hz, 1H), 7.35-7.39 (m, 1H), 6.95 (d, J = 10.8 Hz, 1H), 6.75-6.89 (m, 2H), 6.24 (d, J = 16.8 Hz, 1H), 5.79 (d, J = 10.8 Hz, 1H), 4.74 (s, 2H), 4.06-4.01 (m, 1H), 3.84-3.91 (m, 3H), 2.97-3.03 (m, 2H), 2.67-2.79 (m, 2H). |
| 120 | 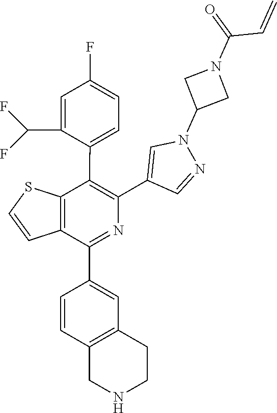 [SMILES: C=CC(=O)N1CC(n2cc(-c3nc(-c4ccc5c(c4)CCNC5)c4ccsc4c3-c3ccc(F)cc3C(F)F)cn2)C1] | LCMS m/z [M + 1]: 586.2 ¹H NMR (400 MHz, CD₃OD) δ 7.86-7.92 (m, 2H), 7.67-7.75 (m, 2H), 7.52-7.66 (m, 4H), 7.47 (d, J = 7.9 Hz, 1H), 7.35 (d, J = 13.2 Hz, 1H), 6.16-6.46 (m, 3H), 5.76 (dd, J = 9.9, 2.3 Hz, 1H), 5.18-5.26 (m, 1H), 4.71 (t, J = 8.9 Hz, 1H), 4.43-4.57 (m, 4H), 4.29 (dd, J = 11.1, 5.2 Hz, 1H), 3.61 (t, J = 6.3 Hz, 2H). |
| 121 | 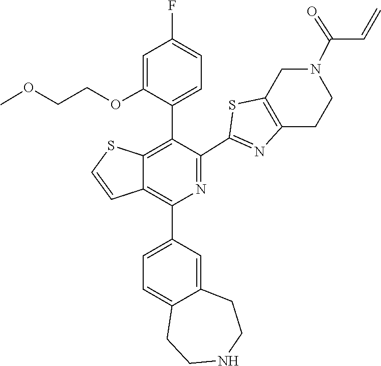 [SMILES: C=CC(=O)N1CCc2nc(-c3nc(-c4ccc5c(c4)CCNCC5)c4ccsc4c3-c3ccc(F)cc3OCCOC)sc2C1] | LCMS m/z [M + 1]: 641.2 ¹H NMR (400 MHz, CD₃OD) δ 7.88-7.76 (m, 3H), 7.71 (d, J = 5.6 Hz, 1H), 7.46 (d, J = 8.0 Hz, 1H), 7.33-7.37 (m, 1H), 6.92 (d, J = 11.2 Hz, 1H), 6.74- 6.93 (m, 2H), 6.24 (d, J = 16.8 Hz, 1H), 5.79 (d, J = 10.0 Hz, 1H), 4.00-4.05 (m, 1H), 3.83-3.89 (m, 3H), 3.39-3.40 (m, 4H), 2.99 (s, 3H), 2.61-2.76 (m, 2H). |
| 122 | 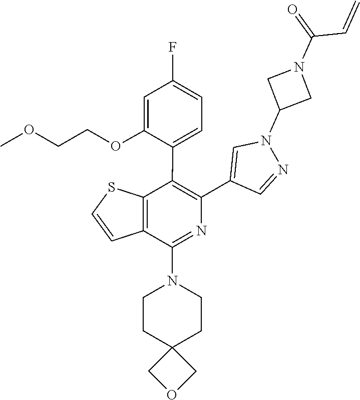 [SMILES: C=CC(=O)N1CC(n2cc(-c3nc(N4CCC5(CC4)COC5)c4ccsc4c3-c3ccc(F)cc3OCCOC)cn2)C1] | LCMS m/z [M + 1]: 604.4 ¹H NMR (400 MHz, CD₃OD) δ 7.86 (d, J = 1.6 Hz, 1H), 7.77-7.81 (m, 2H), 7.53 (d, J = 3.8 Hz, 1H), 7.23 (dd, J = 8.4, 6.6 Hz, 1H), 7.01 (dd, J = 11.1, 2.2 Hz, 1H), 6.84 (dt, J = 8.3, 2.4 Hz, 1H), 6.23-6.40 (m, 2H), 5.77 (dd, J = 9.9, 2.4 Hz, 1H), 5.22-5.28 (m, 1H), 4.74 (t, J = 9.0 Hz, 1H), 4.59 (s, 3H), 4.46-4.55 (m, 3H), 4.27-4.29 (m, 1H), 3.95-4.09 (m, 2H), 3.81- 3.83 (m, 4H), 3.44 (t, J = 4.4 Hz, 2H), 3.12 (s, 3H), 2.19-2.21 (m, 4H). |
| 123 | 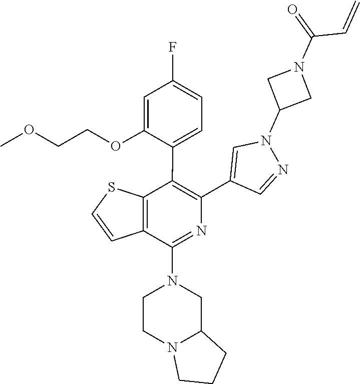 [SMILES: C=CC(=O)N1CC(n2cc(-c3nc(N4CCN5CCCC5C4)c4ccsc4c3-c3ccc(F)cc3OCCOC)cn2)C1] | LCMS m/z [M + 1]: 603.4 ¹H NMR (400 MHz, CD₃OD) δ 7.47-7.56 (m, 4H), 7.21 (t, J = 7.3 Hz, 1H), 7.03 (d, J = 10.9 Hz, 1H), 6.88 (t, J = 8.4 Hz, 1H), 6.25-6.38 (m, 2H), 5.77 (dd, J = 9.8, 1.9 Hz, 1H), 5.18-5.20 (m, 1H), 4.71 (t, J = 9.0 Hz, 1H), 4.44-4.54 (m, 2H), 4.26-4.29 (m, 1H), 3.99-4.10 (m, 3H), 3.57-3.89 (m, 6H), 3.36- 3.38 (m, 2H), 3.16-3.18 (m, 1H), 3.01 (s, 3H), 1.83- 2.29 (m, 5H). |
| 124 | 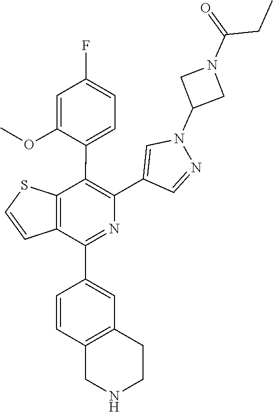 [SMILES: CCC(=O)N1CC(n2cc(-c3nc(-c4ccc5c(c4)CCNC5)c4ccsc4c3-c3ccc(F)cc3OC)cn2)C1] | LCMS m/z [M + 1]: 568.2 ¹H NMR (400 MHz, CD₃OD) δ 7.86 (d, J = 9.9 Hz, 2H), 7.73 (d, J = 5.5 Hz, 1H), 7.69 (s, 1H), 7.63 (d, J = 5.6 Hz, 1H), 7.47 (d, J = 8.8 Hz, 2H), 7.31 (t, J = 8.2 Hz, 1H), 7.05 (dd, J = 11.1, 2.3 Hz, 1H), 6,91 (dt, J = 8.3, 2.4 Hz, 1H), 5.15-5.22 (m, 1H), 4.62 (t, J = 8.8 Hz, 1H), 4.37-4.51 (m, 4H), 4.18-4.23 (m, 1H), 3.67 (s, 3H), 3.61 (t, J = 6.3 Hz, 2H), 2.20 (q, J = 7.6 Hz, 2H), 1.11 (t, J = 7.5 Hz, 3H). |
| 125 | 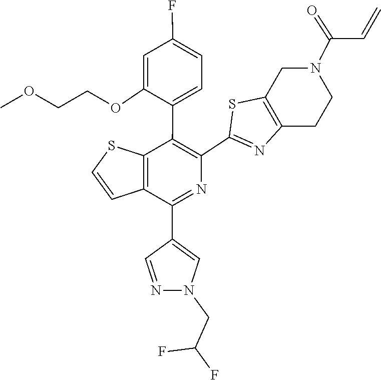 [SMILES: C=CC(=O)N1CCc2nc(-c3nc(-c4cnn(CC(F)F)c4)c4ccsc4c3-c3ccc(F)cc3OCCOC)sc2C1] | LCMS m/z [M + 1]: 626.3 ¹H NMR (400 MHz, CD₃OD) δ 8.57 (s, 1H), 8.35 (s, 1H), 7.86-7.91 (m, 2H), 7.37 (t, J = 6.4 Hz, 1H), 6.75- 7.00 (m, 3H), 6.15-6.45 (m, 2H), 5.75-5.85 (m, 1H), 4.69-4.82 (m, 3H), 4.00-4.08 (m, 1H), 3.80-3.95 (m, 3H), 3.35-3.37 (m, 1H), 3.25-3.30 (m, 1H), 3.00 (s, 3H), 2.70-2.80 (m, 2H). |
| 126 | 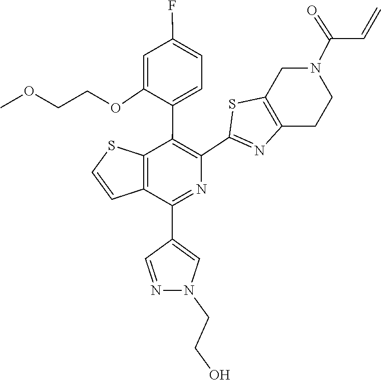 [SMILES: C=CC(=O)N1CCc2nc(-c3nc(-c4cnn(CCO)c4)c4ccsc4c3-c3ccc(F)cc3OCCOC)sc2C1] | LCMS m/z [M + 1]: 605.2 ¹H NMR (400 MHz, CD₃OD) δ 8.55 (s, 1H), 8.31 (s, 1H), 7.94 (dd, J = 16.4, 5.4 Hz, 2H), 7.40 (dd, J = 8.3, 6.7 Hz, 1H), 7.00 (dd, J = 11.0, 1.9 Hz, 1H), 6.72-6.94 (m, 2H), 6.26 (d, J = 16.6 Hz, 1H), 5.80 (t, J = 9.5 Hz, 1H), 4.81 (s, 2H), 4.40 (t, J = 5.3 Hz, 2H), 3.96-4.13 (m, 3H), 3.85-3.95 (m, 3H), 3.31-3.39 (m, 3H), 2.97 (s, 3H), 2.80 (d, J = 3.3 Hz, 2H). |
| 127 | 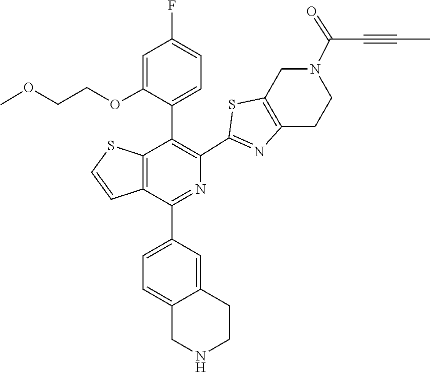 [SMILES: CC#CC(=O)N1CCc2nc(-c3nc(-c4ccc5c(c4)CCNC5)c4ccsc4c3-c3ccc(F)cc3OCCOC)sc2C1] | LCMS m/z [M + 1]: 639.2 ¹H NMR (400 MHz, CD₃OD) δ 7.88-7.90 (m, 2H), 7.80-7.85 (m, 1H), 7.72 (d, J = 5.6 Hz, 1H), 7.45 (d, J = 7.6 Hz, 1H), 7.36 (dd, J = 8.0, 6.8 Hz, 1H), 6.92- 6.95 (m, 1H), 6.83-6.87 (m, 1H), 4.96-5.06 (m, 1H), 4.79 (d, J = 8.0 Hz, 1H), 4.50 (s, 2H), 4.03-4.09 (m, 2H), 3.83-3.93 (m, 2H), 3.61 (t, J = 6.0 Hz, 2H), 3.01 (d, J = 3.2 Hz, 3H), 2.66-2.74 (m, 2H), 2.06 (d, J = 3.6 Hz, 3H). |
| 128 | 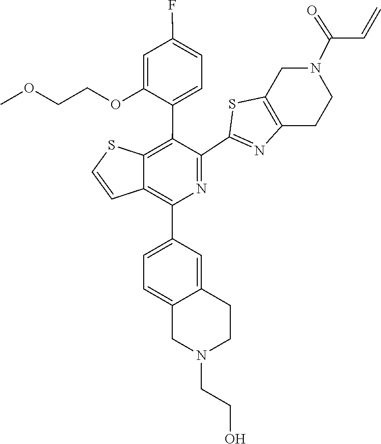 [SMILES: C=CC(=O)N1CCc2nc(-c3nc(-c4ccc5c(c4)CCN(CCO)C5)c4ccsc4c3-c3ccc(F)cc3OCCOC)sc2C1] | LCMS m/z [M + 1]: 671.2 ¹H NMR (400 MHz, CD₃OD) δ 7.89-7.95 (m, 2H), 7.82 (d, J = 5.6 Hz, 1H), 7.72 (d, J = 5.6 Hz, 1H), 7.46 (d, J = 8.6 Hz, 1H), 7.32-7.39 (m, 1H), 6.74-6.97 (m, 3H), 6.19-6.29 (m, 1H), 5.74-5.83 (m, 1H), 4.82- 4.85 (m, 1H), 4.71-4.82 (m, 1H), 4.48-4.61 (m, 1H), 3.99-4.07 (m, 3H), 3.81-3.99 (m, 4H), 3.52-3.63 (m, 1H), 3.49 (t, J = 5.3 Hz, 2H), 3.35-3.45 (m, 2H), 3.32- 3.34 (m, 1H), 2.64-2.77 (m, 2H). |
| 129 | 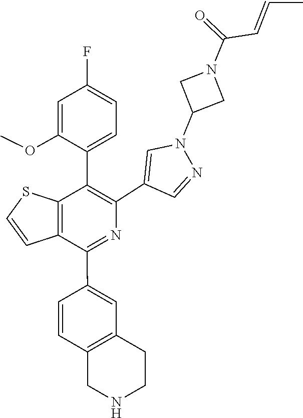 [SMILES: C/C=C/C(=O)N1CC(n2cc(-c3nc(-c4ccc5c(c4)CCNC5)c4ccsc4c3-c3ccc(F)cc3OC)cn2)C1] | LCMS m/z [M + 1]: 580.3 ¹H NMR (400 MHz, CD₃OD) δ 7.85-7.88 (m, 2H), 7.74-7.78 (m, 2H), 7.64-7.65 (m, 1H), 7.48-7.50 (m, 2H), 7.32 (t, J = 7.1 Hz, 1H), 7.04 (d, J = 10.1 Hz, 1H), 6.84-6.93 (m, 2H), 6.04 (d, J = 15.2 Hz, 1H), 5.22 (s, 1H), 4.67-4.71 (m, 2H), 4.43-4.51 (m, 4H), 4.25-4.28 (m, 1H), 3.59-3.67 (m, 5H), 1.90 (d, J = 6.4 Hz, 3H). |
| 130 | 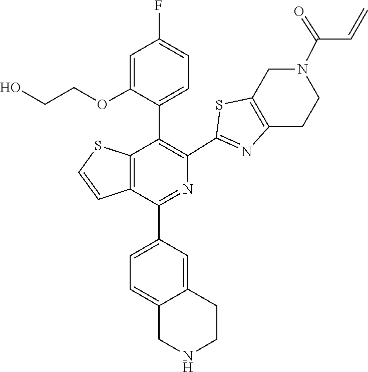 [SMILES: C=CC(=O)N1CCc2nc(-c3nc(-c4ccc5c(c4)CCNC5)c4ccsc4c3-c3ccc(F)cc3OCCO)sc2C1] | LCMS m/z [M + 1]: 613.2 ¹H NMR (400 MHz, CD₃OD) δ 7.91-7.93 (m, 2H), 7.85 (d, J = 5.6 Hz, 1H), 7.73 (d, J = 5.6 Hz, 1H), 7.48 (d, J = 8.0 Hz, 1H), 7.34-7.37 (m, 1H), 6.97 (d, J = 11.2 Hz, 1H), 6.84-6.91 (m, 2H), 6.22-6.28 (m, 1H), 5.78-5.83 (m, 1H), 4.52 (s, 2H), 3.99-4.04 (m, 1H), 3.91-3.94 (m, 2H), 3.80-3.85 (m, 1H), 3.62 (t, J = 6.4 Hz, 2H), 3.46-3.49 (m, 2H), 3.29-3.33 (m, 4H), 2.73- 2.75 (m, 2H). |
| 131 | 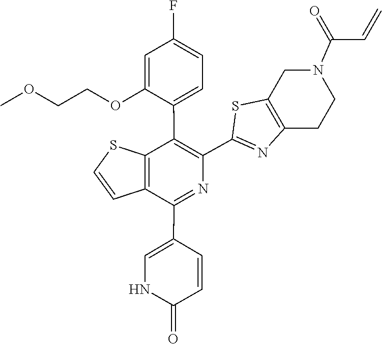 [SMILES: C=CC(=O)N1CCc2nc(-c3nc(-c4ccc(=O)[nH]c4)c4ccsc4c3-c3ccc(F)cc3OCCOC)sc2C1] | LCMS m/z [M + 1]: 589.2 ¹H NMR (400 MHz, CD₃OD) δ 9.08 (s, 2H), 7.89 (d, J = 5.6 Hz, 1H), 7.78 (d, J = 5.6 Hz, 1H), 7.35 (dd, J = 8.3, 6.7 Hz, 1H), 6.95 (d, J = 11.0 Hz, 1H), 6.77- 6.90 (m, 2H), 6.18-6.31 (m, 1H), 5.72-5.85 (m, 1H), 4.78-4.85 (m, 2H), 3.98-4.07 (m, 1H), 3.80-3.95(m, 3H), 3.22-3.30 (m,4H), 2.99 (s, 3H), 2.68-2.77 (m, 2H) |
| 132 | 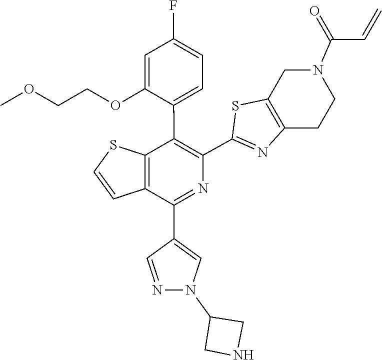 [SMILES: C=CC(=O)N1CCc2nc(-c3nc(-c4cnn(C5CNC5)c4)c4ccsc4c3-c3ccc(F)cc3OCCOC)sc2C1] | LCMS m/z [M + 1]: 617.2 ¹H NMR (400 MHz, CD₃OD) δ 8.56 (s, 1H), 8.49 (s, 1H), 7.92 (d, J = 5.6 Hz, 1H), 7.85 (d, J = 5.6 Hz, 1H), 7.35 (dd, J = 8.3, 6.7 Hz, 1H), 6.77-6.96 (m, 3H), 6.17-6.29 (m, 1H), 5.75-5.83 (m, 1H), 5.55-5.64 (m, 1H), 4.87-4.92 (m, 2H), 4.59-4.70 (m, 4H), 3.98- 4.06 (m, 1H), 3.82-3.95 (m, 3H), 3.23-3.30 (m, 2H), 2.98 (s, 3H), 2.68-2.78 (m, 2H). |
| 133 | 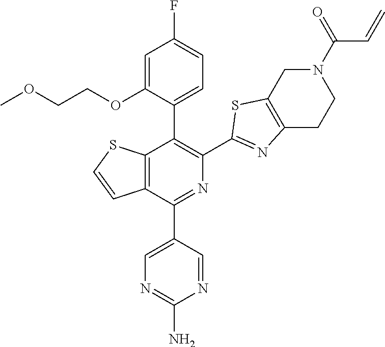 [SMILES: C=CC(=O)N1CCc2nc(-c3nc(-c4cnc(N)nc4)c4ccsc4c3-c3ccc(F)cc3OCCOC)sc2C1] | LCMS m/z [M + 1]: 589.2 ¹H NMR (400 MHz, CD₃OD) δ 9.08 (s, 2H), 7.89 (d, J = 5.6 Hz, 1H), 7.78 (d, J = 5.6 Hz, 1H), 7.35 (dd, J = 8.3, 6.7 Hz, 1H), 6.95 (d, J = 11.0 Hz, 1H), 6.77- 6.90 (m, 2H), 6.18-6.31 (m, 1H), 5.72-5.85 (m, 1H), 4.78-4.85 (m, 2H), 3.98-4.07 (m, 1H), 3.80-3.95 (m, 3H), 3.22-3.30 (m,4H), 2.99 (s, 3H), 2.68-2.77 (m, 2H). |
| 134 | 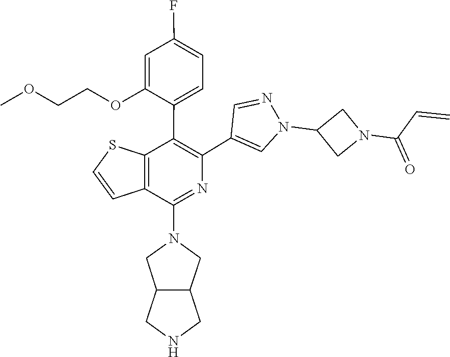 [SMILES: C=CC(=O)N1CC(n2cc(-c3nc(N4CC5CNCC5C4)c4ccsc4c3-c3ccc(F)cc3OCCOC)cn2)C1] | LCMS m/z [M + 1]: 589.2 ¹H NMR (400 MHz, CD₃OD) δ 7.93 (d, J = 5.6 Hz, 1H), 7.72 (s, 1H), 7.63 (d, J = 5.2 Hz, 1H), 7.54 (d, J = 4.4 Hz, 1H), 7.20-7.23 (m, 1H), 7.02 (dd, J = 11.2, 2.0 Hz, 1H), 6.83-6.88 (m, 1H), 6.26-6.40 (m, 2H), 5.78 (dd, J = 10.0, 2.4 Hz, 1H), 5.20-5.26 (m, 1H), 4.74 (t, J = 8.8 Hz, 1H), 4.47-4.56 (m, 2H), 4.21-4.31 (m, 3H), 4.10-4.13 (m, 2H), 4.02-4.05 (m, 2H), 3.67- 3.72 (m, 2H), 3.41-3.45 (m, 6H), 3.10 (s, 3H). |
| 135 | 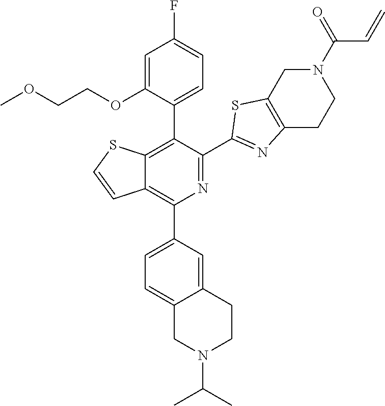 [SMILES: C=CC(=O)N1CCc2nc(-c3nc(-c4ccc5c(c4)CCN(C(C)C)C5)c4ccsc4c3-c3ccc(F)cc3OCCOC)sc2C1] | LCMS m/z [M + 1]: 669.2 ¹H NMR (400 MHz, CD₃OD) δ 7.90-7.97 (m, 2H), 7.83 (d, J = 5.6 Hz, 1H), 7.72 (d, J = 5.6 Hz, 1H), 7.48 (d, J = 8.4 Hz, 1H), 7.36 (t, J = 8.0 Hz, 1H), 6.94 (d, J = 10.8 Hz, 1H), 6.82-6.88 (m, 2H), 6.24 (d, J = 16.8 Hz, 1H), 5.79 (d, J = 9.6 Hz, 1H), 4.59 (s, 2H), 4.01- 4.05 (m, 1H), 3.77-3.90 (m, 5H), 3.30-3.58 (m, 4H), 3.13 (s, 3H), 3.65-3.77 (m, 2H), 1.52 (d, J = 6.4 Hz, 6H). |
| 136 | 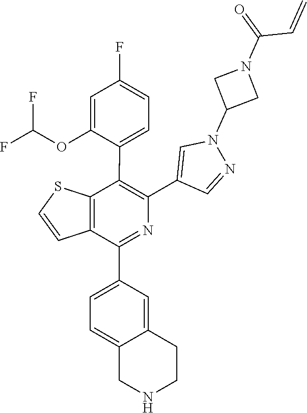 [SMILES: C=CC(=O)N1CC(n2cc(-c3nc(-c4ccc5c(c4)CCNC5)c4ccsc4c3-c3ccc(F)cc3OC(F)F)cn2)C1] | LCMS m/z [M + 1]: 602.2 ¹H NMR (400 MHz, CD₃OD) δ 7.85-7.90 (m, 2H), 7.64-7.77 (m, 3H), 7.50-7.56 (m, 1H), 7.42-7.49 (m, 2H), 7.22-7.28 (m, 2H), 6.72 (t, J = 7.3 Hz, 1H), 6.24- 6.40 (m, 2H), 5.77 (dd, J = 10.0, 2.3 Hz, 1H), 5.20- 5.28 (m, 1H), 4.73 (t, J = 8.7 Hz, 1H), 4.44-4.60 (m, 4H), 4.28-4.34 (m, 1H), 3.60 (t, J = 6.3 Hz, 2H). |
| 137 | 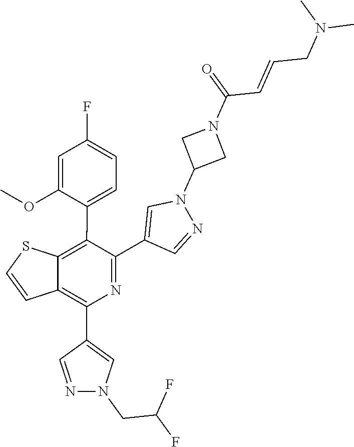 | LCMS m/z [M + 1]: 622.2 ¹H NMR (400 MHz, CD₃OD) δ 8.64 (s, 1H), 8.36 (s, 1H), 7.91-7.94 (m, 3H), 7.50 (d, J = 4.6 Hz, 1H), 7.32-7.36 (m, 1H), 7.03 (dd, J = 11.0 , 2.2 Hz, 1H), 6.88-6.93 (m, 1H), 6.69-6.80 (m, 1H), 6.18-6.54 (m, 2H), 5.27-5.33 (m, 1H), 4.72-4.80 (m, 3H), 4.50- 4.63 (m, 2H), 4.31-4.36 (m, 1H), 3.95 (d, J = 7.1 Hz, 2H), 3.66 (s, 3H), 2.91 (s, 6H). |
| 138 | 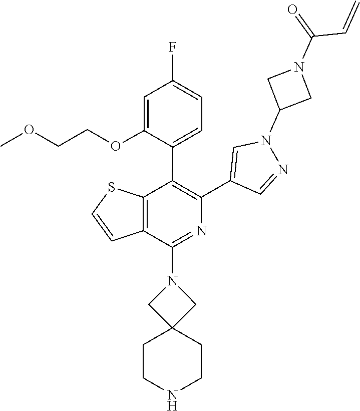 [SMILES: C=CC(=O)N1CC(n2cc(-c3nc(N4CC5(CCNCC5)C4)c4ccsc4c3-c3ccc(F)cc3OCCOC)cn2)C1] | LCMS m/z [M + 1]: 603.3 ¹H NMR (400 MHz, CD₃OD) δ 7.88 (s, 1H), 7.78- 7.82 (m, 2H), 7.55 (d, J = 3.2 Hz, 1H), 7.19-7.23 (m, 1H), 6.98 (dd, J = 11.0, 2.1 Hz, 1H), 6.78-6.83 (m, 1H), 6.25-6.39 (m, 2H), 5.77 (dd, J = 9.8, 2.3 Hz, 1H), 5.22-5.28 (m, 1H), 4.74 (t, J = 9.0 Hz, 1H), 4.47-4.57 (m, 6H), 4.24-4.30 (m, 1H), 3.99-4.07 (m, 2H), 3.45-3.48 (m, 2H), 3.27-3.31 (m, 2H), 3.16 (s, 3H), 2.20-2.23 (m, 4H). |
| 139 | 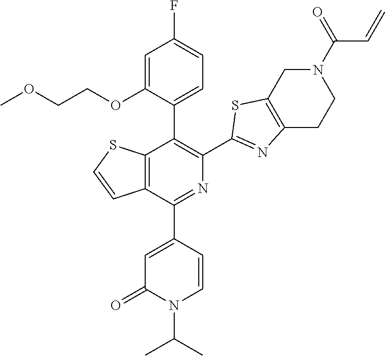 [SMILES: C=CC(=O)N1CCc2nc(-c3nc(-c4ccn(C(C)C)c(=O)c4)c4ccsc4c3-c3ccc(F)cc3OCCOC)sc2C1] | LCMS m/z [M + 1]: 631.2 ¹H NMR (400 MHz, CD₃OD) δ 7.94 (d, J = 7.2 Hz, 1H), 7.89 (d, J = 5.6 Hz, 1H), 7.78 (d, J = 5.6 Hz, 1H), 7.36 (t, J = 8.0 Hz, 1H), 7.07-7.10 (m, 2H), 6.83- 6.93 (m, 3H), 6.24 (d, J = 16.4 Hz, 1H), 5.79 (d, J = 10.8 Hz, 1H), 5.27-5.30 (m, 1H), 4.00-4.02 (m, 1H), 3.80-3.98 (m, 3H), 2.99 (s, 3H), 2.60-2.75 (m, 2H), 1.48-1.50 (m, 6H). |
| 140 | 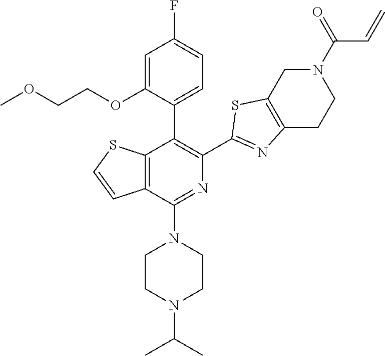 [SMILES: C=CC(=O)N1CCc2nc(-c3nc(N4CCN(C(C)C)CC4)c4ccsc4c3-c3ccc(F)cc3OCCOC)sc2C1] | LCMS m/z [M + 1]: 622.2 ¹H NMR (400 MHz, CD₃OD) δ 7.74 (d, J = 5.6 Hz, 1H), 7.65 (d, J = 5.6 Hz, 1H), 7.24-7.28 (m, 1H), 6.75-6.90 (m, 3H), 6.24 (d, J = 16.8 Hz, 1H), 5.79 (d, J = 10.4 Hz, 1H), 4.39-4.41 (m, 2H), 3.98-4.01 (m, 1H), 3.82-3.89 (m, 3H), 3.60-3.65 (m, 3H), 3.47-3.49 (m, 4H), 3.24-3.29 (m, 4H), 3.00 (s, 3H), 2.61-2.73 (m, 2H), 1.46 (d, J = 6.4 Hz, 6H). |
| 141 | 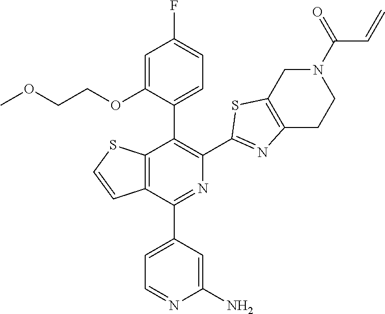 [SMILES: C=CC(=O)N1CCc2nc(-c3nc(-c4ccnc(N)c4)c4ccsc4c3-c3ccc(F)cc3OCCOC)sc2C1] | LCMS m/z [M + 1]: 588.2 ¹H NMR (400 MHz, CD₃OD) δ 8.04 (d, J = 6.7 Hz, 1H), 7.96 (d, J = 5.6 Hz, 1H), 7.82 (d, J = 5.6 Hz, 1H), 7.63 (s, 1H), 7.52 (dd, J = 6.7, 1.3 Hz, 1H), 7.37 (dd, J = 8.3, 6.6 Hz, 1H), 6.94 (d, J = 11.1 Hz, 1H), 6.75-6.90 (m, 2H), 6.19-6.30 (m, 1H), 5.76-5.83 (m, 1H), 4.88-4.95 (m, 2H), 4.70-4.86 (m, 2H), 3.99-4.07 (m, 1H), 3.80-3.94 (m, 3H), 3.21-3.30 (m, 2H), 2.99 (s, 3H), 2.65-2.75 (m, 2H). |
| 142 | 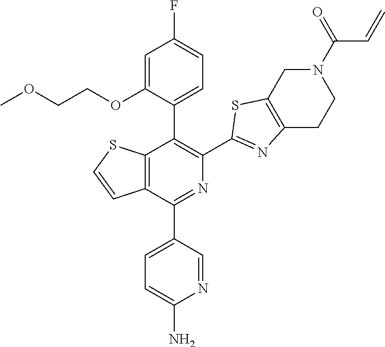 [SMILES: C=CC(=O)N1CCc2nc(-c3nc(-c4ccc(N)nc4)c4ccsc4c3-c3ccc(F)cc3OCCOC)sc2C1] | LCMS m/z [M + 1]: 588.2 ¹H NMR (400 MHz, CD₃OD) δ 8.63 (d, J = 8 Hz, 1H), 8.51 (s, 1H), 7.90 (d, J = 4 Hz, 1H), 7.79 (d, J = 4 Hz, 1H), 7.35 (t, J = 8 Hz, 1H), 7.24 (d, J = 8 Hz, 1H), 6.76-6.98 (m, 3H), 6.23-6.27 (m, 1H), 5.79-5.81 (m, 1H), 4.02-4.03 (m, 1H), 3.87-3.91 (m, 2H), 3.00 (s, 3H), 2.66-2.73 (m, 2H). |
| 143 | 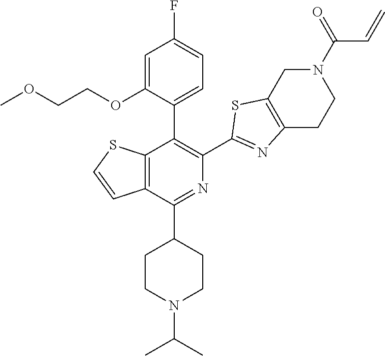 [SMILES: C=CC(=O)N1CCc2nc(-c3nc(C4CCN(C(C)C)CC4)c4ccsc4c3-c3ccc(F)cc3OCCOC)sc2C1] | LCMS m/z [M + 1]: 621.3 ¹H NMR (400 MHz, CD₃OD) δ 7.77-7.82 (m, 2H), 7.29 (t, J = 5.9 Hz, 1H), 6.80-6.91 (m, 3H), 6.25 (d, J = 16 Hz, 1H), 5.80 (d, J = 10.4 Hz, 1H), 3.99-4.03 (m, 1H), 3.88-3.91 (m, 2H), 3.82-3.85 (m, 2H), 3.61- 3.79 (m, 3H), 3.35-3.39 (m, 2H), 3.25-3.35 (m, 2H), 3.06 (s, 3H), 2.63-2.71 (m, 2H), 2.63-2.71 (m, 2H), 2.31-2.49 (m, 4H), 1.45 (d, J = 6.4 Hz, 1H). |
| 144 | 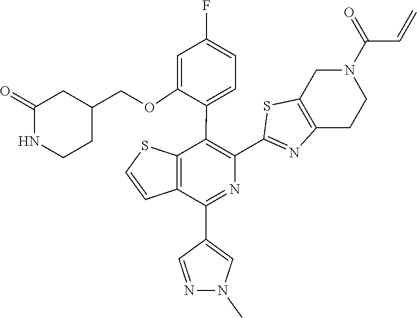 [SMILES: C=CC(=O)N1CCc2nc(-c3nc(-c4cnn(C)c4)c4ccsc4c3-c3ccc(F)cc3OCC3CCNC(=O)C3)sc2C1] | LCMS m/z [M + 1]: 629.3 ¹H NMR (400 MHz, CD₃OD) δ 8.53 (d, J = 6.2 Hz, 1H), 8.31 (d, J = 4.2 Hz, 1H), 7.94-7.99 (m, 2H), 7.39 (t, J = 8.8 Hz, 1H), 6.89-7.03 (m, 3H), 6.25 (d, J = 16.7 Hz, 1H), 5.80 (d, J = 10.0 Hz, 1H), 4.07 (s, 3H), 3.79-3.93 (m, 4H), 2.99-3.01 (m, 2H), 2.82-2.86 (m, 2H), 1.97-2.03 (m, 2H), 1.47-1.73 (m, 2H), 1.08-1.20 (m, 1H). |
| 145 | 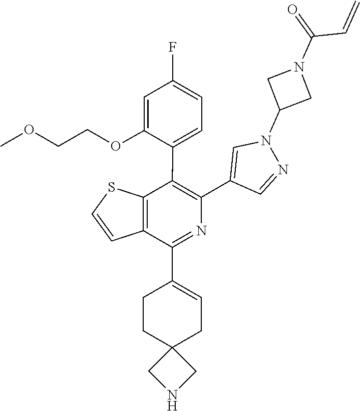 [SMILES: C=CC(=O)N1CC(n2cc(-c3nc(C4=CCC5(CC4)CNC5)c4ccsc4c3-c3ccc(F)cc3OCCOC)cn2)C1] | LCMS m/z [M + 1]: 600.3 ¹H NMR (400 MHz, CD₃OD) δ 7.68-7.77 (m, 3H), 7.54 (d, J = 4.0 Hz, 1H), 7.28-7.32 (m, 1H), 7.06 (dd, J = 10.8, 2.0 Hz, 1H), 6.89-6.93 (m, 1H), 6.27-6.41 (m, 3H), 5.79 (dd, J = 9.6, 2.4 Hz, 1H), 5.21-5.28 (m, 1H), 4.75 (t, J = 8.4 Hz, 1H), 4.48-4.58 (m, 2H), 4.28- 4.32 (m, 1H), 3.97-4.08 (m, 6H), 3.42 (t, J = 4.8 Hz, 2H), 3.06 (d, J = 2.4 Hz, 3H), 2.85-2.90 (m, 2H), 2.72-2.78 (m, 2H) 2.22 (t, J = 6.0 Hz, 2H). |
| 146 | 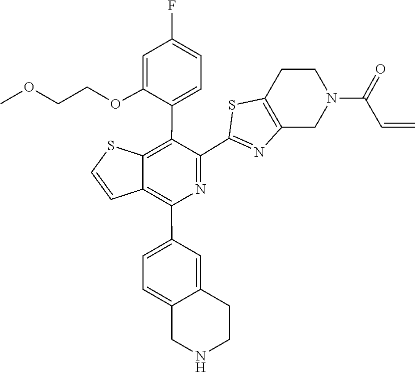 [SMILES: C=CC(=O)N1CCc2sc(-c3nc(-c4ccc5c(c4)CCNC5)c4ccsc4c3-c3ccc(F)cc3OCCOC)nc2C1] | LCMS m/z [M + 1]: 627.2 ¹H NMR (400 MHz, CD₃OD) δ 7.87-7.92 (m, 2H), 7.80-7.84 (m, 1H), 7.70-7.73 (m, 1H), 7.46 (d, J = 7.9 Hz, 1H), 7.36 (t, J = 7.6 Hz, 1H), 6.95 (dd, J = 11.2, 2.1 Hz, 1H), 6.72-6.90 (m, 2H), 6.18-6.27 (m, 1H), 5.79 (d, J = 10.3 Hz, 1H), 4.47-4.64 (m, 4H), 4.01- 4.08 (m, 1H), 3.83-3.99 (m, 3H), 3.61 (t, J = 6.4 Hz, 2H), 3.00 (s, 3H), 2.88-2.97 (m, 2H). |
| 147 | 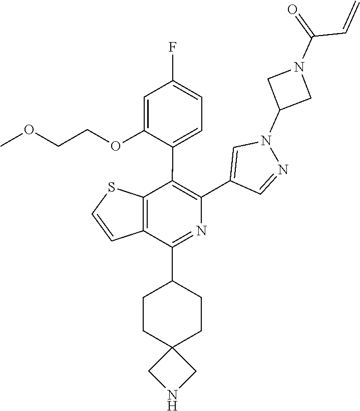 [SMILES: C=CC(=O)N1CC(n2cc(-c3nc(C4CCC5(CC4)CNC5)c4ccsc4c3-c3ccc(F)cc3OCCOC)cn2)C1] | LCMS m/z [M + 1]: 602.3 ¹H NMR (400 MHz, CD₃OD) δ 7.69-7.78 (m, 3H), 7.52 (d, J = 3.7 Hz, 1H), 7.28 (t, J = 7.2 Hz, 1H), 7.04 (dd, J = 11.0, 2.0 Hz, 1H), 6.87-6.92 (m, 1H), 6.25-6.39 (m, 2H), 5.77 (dd, J = 9.8, 2.2 Hz, 1H), 5.20-5.27 (m, 1H), 4.73 (t, J = 8.8 Hz, 1H), 4.46-4.56 (m, 2H), 4.27-4.31 (m, 1H), 3.97-4.05 (m, 6H), 3.40 (t, J = 4.4 Hz, 2H), 3.05 (s, 3H), 2.84 (s, 2H), 2.75 (s, 2H), 2.20 (t, J = 6.2 Hz, 2H). |
| 148 | 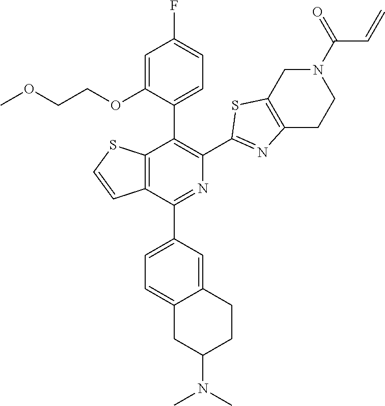 [SMILES: C=CC(=O)N1CCc2nc(-c3nc(-c4ccc5c(c4)CCC(N(C)C)C5)c4ccsc4c3-c3ccc(F)cc3OCCOC)sc2C1] | LCMS m/z [M + 1]: 669.2 ¹H NMR (400 MHz, CD₃OD) δ 7.77-7.82 (m, 3H), 7.70 (d, J = 5.6 Hz, 1H), 7.35-7.43 (m, 2H), 6.74-6.96 (m, 3H), 6.22-6.26 (m, 1H), 5.77-5.81 (m, 1H), 3.73- 4.06 (m, 5H), 3.31-3.39 (m, 2H), 3.07-3.22 (m, 3H), 2.99-3.01 (m, 9H), 2.74 (s, 2H), 2.41-2.43 (m, 1H), 1.96-2.06 (m, 1H). |
| 149 | 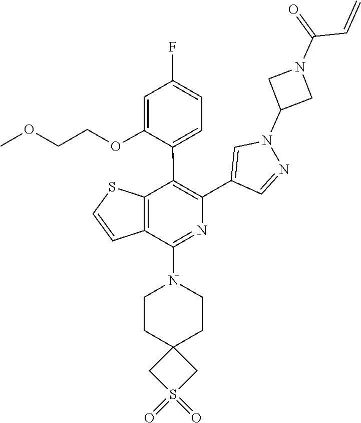 [SMILES: C=CC(=O)N1CC(n2cc(-c3nc(N4CCC5(CC4)CS(=O)(=O)C5)c4ccsc4c3-c3ccc(F)cc3OCCOC)cn2)C1] | LCMS m/z [M + 1]: 652.2 ¹H NMR (400 MHz, CD₃OD) δ 7.52 (d, J = 4.4 Hz, 1H), 7.47 (t, J = 6.0 Hz, 2H), 7.39 (d, J = 4.8 Hz, 1H), 7.21 (t, J = 7.6 Hz, 1H), 7.00 (dd, J = 11.1, 2.3 Hz 1H), 6.83-6.88 (m, 1H), 6.24-6.38 (m, 2H), 5.74-5.77 (m, 1H), 5.16-5.21 (m, 1H), 4.70 (t, J = 8.8 Hz, 1H), 444-4.58 (m, 2H), 4.27-4.30 (m, 1H), 3.96-4.03 (m, 6H), 3.57-3.59 (m, 4H), 2.99 (s, 3H), 2.11-2.14 (m, 4H). |
| 150 | 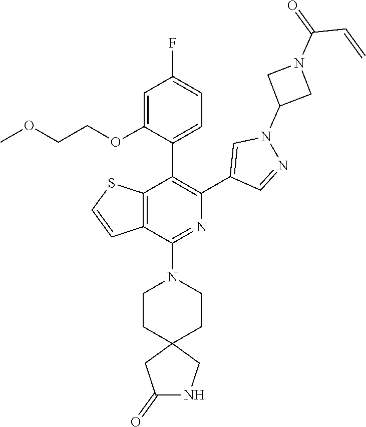 [SMILES: C=CC(=O)N1CC(n2cc(-c3nc(N4CCC5(CC4)CNC(=O)C5)c4ccsc4c3-c3ccc(F)cc3OCCOC)cn2)C1] | LCMS m/z [M + 1]: 631.3 ¹H NMR (400 MHz, CD₃OD) δ 7.89 (s, 1H), 7.83 (s, 2H), 7.54 (d, J = 4.4 Hz, 1H), 7.22-7.26 (m, 1H), 6.99-7.03 (m, 1H), 6.84-6.86 (m, 1H), 6.26-6.35 (m, 2H), 5.75 (dd, J = 9.9, 2.3 Hz 1H), 5.24-5.28 (m, 1H), 4.74 (t, J = 8.9 Hz), 4.47-4.56 (m, 2H), 4.27-4.30 (m, 1H), 3.97-4.05 (m, 4H), 3.86-3.89 (m, 2H), 3.45 (t, J = 4.4 Hz), 3.30-3.31 (m, 4H), 3.14 (s, 3H), 2.00-2.02 (m, 4H). |
| 151 | 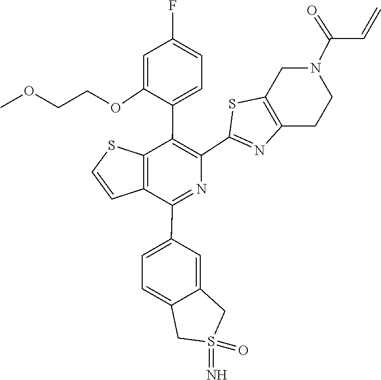 [SMILES: C=CC(=O)N1CCc2nc(-c3nc(-c4ccc5c(c4)CS(=N)(=O)C5)c4ccsc4c3-c3ccc(F)cc3OCCOC)sc2C1] | LCMS m/z [M + 1]: 661.2 ¹H NMR (400 MHz, CD₃OD) δ 8.02-8.09 (m, 2H), 7.84 (d, J = 4.8 Hz, 1H), 7.74 (d, J = 5.6 Hz, 1H), 7.66 (d, J = 8.0 Hz, 1H), 7.37 (dd, J = 8.4, 6.4 Hz, 1H), 6.94 (d, J = 11.2 Hz, 1H), 6.82-6.88 (m, 2H), 6.24 (d, J = 16.4 Hz, 1H), 5.80 (d, J = 10.8 Hz, 1H), 5.05 (s, 2H), 5.02 (s, 2H), 4.00-4.06 (m, 1H), 3.82-3.95 (m, 3H), 3.00 (s, 3H), 2.65-2.72 (m, 2H). |
| 152 | 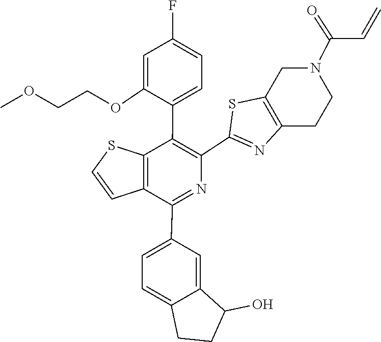 [SMILES: C=CC(=O)N1CCc2nc(-c3nc(-c4ccc5c(c4)C(O)CC5)c4ccsc4c3-c3ccc(F)cc3OCCOC)sc2C1] | LCMS m/z [M + 1]: 628.4 1H NMR (400 MHz, CD3OD) δ 7.98 (s, 1H), 7.88 (dd, J = 11.9, 6.7 Hz, 2H), 7.80 (d, J = 5.6 Hz, 1H), 7.50 (d, J = 7.8 Hz, 1H), 7.38-7.45 (m, 1H), 6.99 (dd, J = 11.1, 1.9 Hz, 1H), 6.77-6.92 (m, 2H), 6.24 (d, J = 17.0 Hz, 1H), 5.73-5.85 (m, 1H), 5.32 (t, J = 6.2 Hz, 1H), 4.85-4.92 (m, 1H), 4.01-4.08 (m, 1H), 3.84-3.96 (m, 3H), 3.32-3.39 (m, 3H),3.13-3.21 (m, 1H), 3.00 (s, 3H), 2.90-2.97 (m, 1H), 2.71-2.84 (m, 2H), 2.51- 2.62 (m, 1H), 1.98-2.10 (m, 1H). |
| 153 | 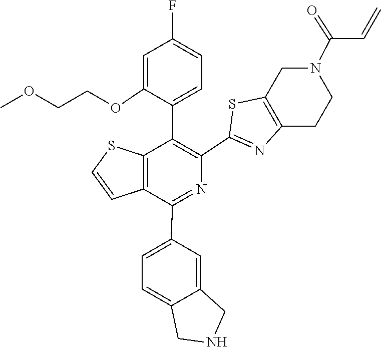 [SMILES: C=CC(=O)N1CCc2nc(-c3nc(-c4ccc5c(c4)CNC5)c4ccsc4c3-c3ccc(F)cc3OCCOC)sc2C1] | LCMS m/z [M + 1]: 613.3 ¹H NMR (400 MHz, CD₃OD) δ 8.00-8.07 (m, 2H), 7.83 (d, J = 5.6 Hz, 1H), 7.72 (d, J = 5.6 Hz, 1H), 7.65 (d, J = 7.9 Hz, 1H), 7.36 (dd, J = 8.3, 6.7 Hz, 1H), 6.91-6.96 (m, 1H), 6.73-6.90 (m, 2H), 6.16-6.30 (m, 1H), 5.73-5.85 (m, 1H), 4.77 (d, J = 7.9 Hz, 4H), 3.97-4.09 (m, 1H), 3.81-3.95 (m, 3H), 3.26-3.30 (m, 3H), 3.00 (s, 3H), 2.66-2.77 (m, 2H). |
| 154 | 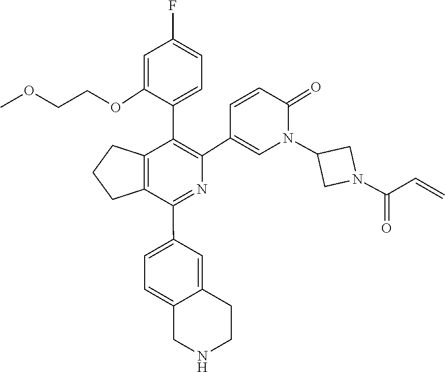 [SMILES: C=CC(=O)N1CC(n2cc(-c3nc(-c4ccc5c(c4)CCNC5)c4c(c3-c3ccc(F)cc3OCCOC)CCC4)ccc2=O)C1] | LCMS m/z [M + 1]: 621.3 ¹H NMR (400 MHz, CD₃OD) δ 8.19 (dd, J = 5.5, 2.3 Hz, 1H), 7.70-7.16 (m, 3H), 7.46 (d, J = 8.6 Hz, 1H), 7.03-7.07 (m, 1H), 6.91 (dd, J = 11.0, 1.3 Hz, 1H), 6.81 (d, J = 8.6 Hz, 1H), 6.71 (t, J = 8.6 Hz, 1H), 6.22- 6.37 (m, 2H), 5.74 (dd, J = 10.1, 2.1 Hz, 1H), 5.39- 5.41 (m, 1H), 4.68 (t, J = 9.7 Hz, 1H), 4.41-4.48 (m, 3H), 4.26 (d, J = 10.21 Hz, 1H), 4.09-4.14 (m, 1H), 3.97-4.02 (m, 2H), 3.56-3.61 (m, 4H), 3.24-3.30 (m, 7H), 2.85-3.04 (m, 2H), 2.15-2.22 (m, 2H). |
| 155 | 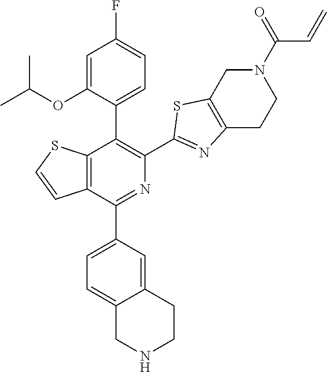 [SMILES: C=CC(=O)N1CCc2nc(-c3nc(-c4ccc5c(c4)CCNC5)c4ccsc4c3-c3ccc(F)cc3OC(C)C)sc2C1] | LCMS m/z [M + 1]: 611.2 ¹H NMR (400 MHz, CD₃OD) δ 7.90-7.93 (m, 2H), 7.83 (d, J = 5.6 Hz, 1H), 7.72 (d, J = 5.6 Hz, 1H), 7.46 (d, J = 8.2 Hz, 1H), 7.36 (dd, J = 8.2, 6.9 Hz, 1H), 6.79-6.89 (m, 3H), 6.24 (d, J = 16.7 Hz, 1H), 5.80 (d, J = 9.9 Hz, 1H), 4.50 (s, 2H), 4.43-4.48 (m, 1H), 3.89-3.91 (m, 2H), 3.61 (t, J = 6.3 Hz, 2H), 3.24- 3.32(m, 4H), 2.65-2.76 (m, 2H), 1.04 (d, J = 6.0 Hz, 3H), 0.85 (d, J = 6.0 Hz, 3H). |
| 156 | 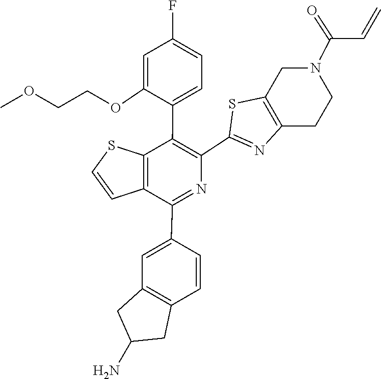 [SMILES: C=CC(=O)N1CCc2nc(-c3nc(-c4ccc5c(c4)CC(N)C5)c4ccsc4c3-c3ccc(F)cc3OCCOC)sc2C1] | LCMS m/z [M + 1]: 627.2 ¹H NMR (400 MHz, CD₃OD) δ 7.94 (s, 1H), 7.88 (d, J = 8.0 Hz, 1H), 7.84 (d, J = 5.6 Hz, 1H), 7.74 (d, J = 5.6 Hz, 1H), 7.56 (d, J = 8.0 Hz, 1H), 7.39 (t, J = 7.6 Hz, 1H), 6.96 (d, J = 10.8 Hz, 1H), 6.77-6.91 (m, 2H), 6.23-6.29 (m, 1H), 5.79-5.84 (m, 1H), 4.21-4.27 (m, 1H), 4.04-4.09 (m, 1H), 3.87-3.95 (m, 3H), 3.55-3.63 (m, 2H), 3.30-3.39 (m, 4H), 3.15-3.23 (m, 2H), 3.03 (m, 3H), 2.70-2.79 (m, 2H). |
| 157 | 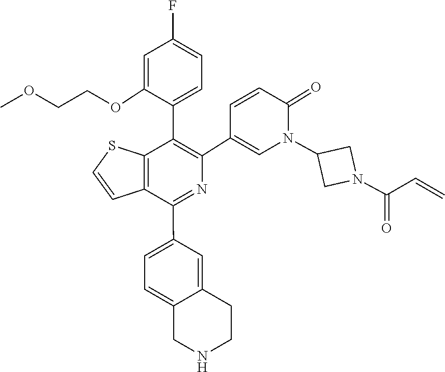 [SMILES: C=CC(=O)N1CC(n2cc(-c3nc(-c4ccc5c(c4)CCNC5)c4ccsc4c3-c3ccc(F)cc3OCCOC)ccc2=O)C1] | LCMS m/z [M + 1]: 637.4 ¹H NMR (400 MHz, CD₃OD) δ 8.24 (s, 1H), 7.89 (d, J = 5.5 Hz, 1H), 7.81-7.87 (m, 3H), 7.71 (d, J = 5.7 Hz, 1H), 7.49 (d, J = 8.0 Hz, 1H), 7.31-7.35 (m, 1H), 6.95 (dd, J = 11.0, 1.9 Hz, 1H), 6.82 (t, J = 9.2 Hz, 2H), 6.22-6.37 (m, 2H), 5.74 (dd, J = 10.1, 2.0 Hz, 1H), 5.39-5.41 (m, 1H), 4.69 (t, J = 10.0 Hz, 1H), 4.51 (s, 2H), 4.42-4.47 (m, 1H), 4.27 (d, J = 10.3 Hz, 1H), 3.91-4.06 (m, 3H), 3.60 (t, J = 6.4 Hz, 2H), 3.46 (d, J = 4.6 Hz, 2H), 3.28 (d, J = 5.7 Hz, 2H), 3.14 (s, 3H). |
| 158 | 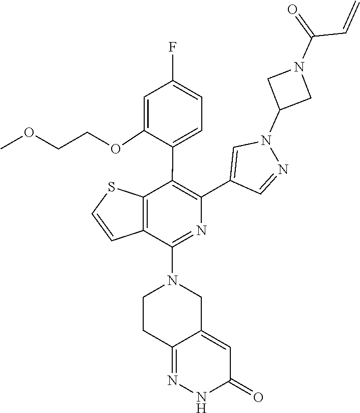 [SMILES: C=CC(=O)N1CC(n2cc(-c3nc(N4CCc5n[nH]c(=O)cc5C4)c4ccsc4c3-c3ccc(F)cc3OCCOC)cn2)C1] | LCMS m/z [M + 1]: 628.4 ¹H NMR (400 MHz, CD₃OD) δ 7.60 (d, J = 5.6 Hz, 1H), 7.52-7.58 (m, 2H), 7.34 (d, J = 4.0 Hz, 1H), 7.21-7.27 (m, 1H), 7.01 (dd, J = 11.1, 2.1 Hz, 1H), 6.96 (s, 1H), 6.82-6.91 (m, 1H), 6.31-6.40 (m, 1H), 6.23-6.30 (m, 1H), 5.76 (dd, J = 10.0, 2.1 Hz, 1H), 5.15-5.24 (m, 1H), 4.80 (s, 2H), 4.71 (t, J = 8.7 Hz, 1H), 4.44-4.57 (m, 2H), 4.28 (dd, J = 10.7, 5.1 Hz, 1H), 3.92-4.05 (m, 4H), 3.37 (t, J = 4.6 Hz, 2H), 2.98- 3.05 (m, 2H), 2.98 (s, 3H). |
| 159 | 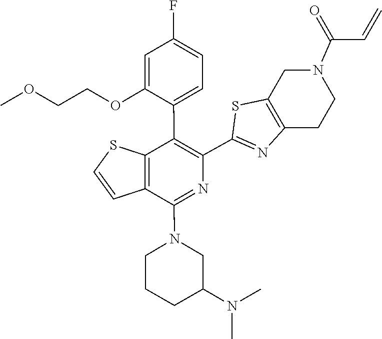 [SMILES: C=CC(=O)N1CCc2nc(-c3nc(N4CCCC(N(C)C)C4)c4ccsc4c3-c3ccc(F)cc3OCCOC)sc2C1] | LCMS m/z [M + 1]: 622.2 ¹H NMR (400 MHz, CD₃OD) δ 7.73 (d, J = 5.6 Hz, 1H), 7.63 (d, J = 5.6 Hz, 1H), 7.24-7.32 (m, 1H), 6.75-7.01 (m, 3H), 6.23-6.29 (m, 1H), 5.73-5.78 (m, 1H), 4.84 (s, 2H), 4.17-4.29 (m, 1H), 3.88-4.03 (m, 5H), 3.70-3.76 (m, 2H), 3.50-3.60 (m, 1H), 3.28-3.33 (m, 2H), 3.02-3.09 (m, 9H), 2.83 (t, J = 5.6 Hz, 2H), 2.25-2.35 (m, 1H), 1.85-2.05 (m, 3H). |
| 160 | 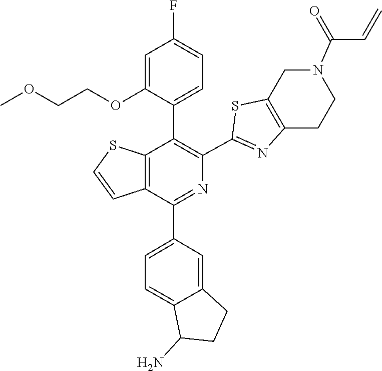 [SMILES: C=CC(=O)N1CCc2nc(-c3nc(-c4ccc5c(c4)CCC5N)c4ccsc4c3-c3ccc(F)cc3OCCOC)sc2C1] | LCMS m/z [M + 1]: 627.3 ¹H NMR (400 MHz, CD₃OD) δ 7.90-7.99 (m, 2H), 7.82 (d, J = 5.5 Hz, 1H), 7.66-7.74 (m, 2H), 7.36 (dd, J = 8.1, 6.8 Hz, 1H), 6.77-6.98 (m, 3H), 6.18-6.29 (m, 1H), 5.75-5.84 (m, 1H), 4.87-4.95 (m, 2H), 4.00-4.08 (m, 1H), 3.82-3.97 (m, 3H), 3.32-3.39 (m, 2H), 3.11- 3.25 (m, 2H), 3.00 (s, 3H), 2.63-2.81 (m, 3H), 2.16- 2.26 (m, 1H). |
| 161 | 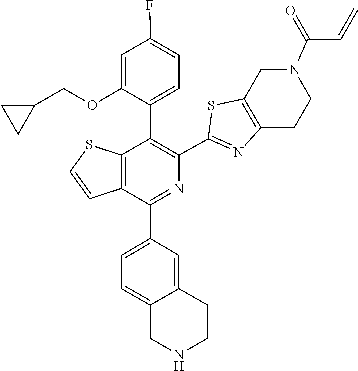 [SMILES: C=CC(=O)N1CCc2nc(-c3nc(-c4ccc5c(c4)CCNC5)c4ccsc4c3-c3ccc(F)cc3OCC3CC3)sc2C1] | LCMS m/z [M + 1]: 623.3 ¹H NMR (400 MHz, CD₃OD) δ7.89-7.91 (m, 2H), 7.82 (d, J = 5.6 Hz, 1H), 7.71 (d, J = 5.6 Hz, 1H), 7.45 (d, J = 8.4 Hz, 1H), 7.33 (t, J = 7.2 Hz, 1H), 6.70-6.90 (m, 3H), 6.18-6.28 (m, 1H), 5.75-5.85 (m, 1H), 4.80- 4.85 (m, 2H), 4.50 (s, 2H), 3.85-3.95 (m, 2H), 3.70- 3.80 (m, 1H), 3.52-3.65 (m, 3H), 3.25-3.30 (m, 2H), 2.60-2.75 (m, 2H), 0.75-0.95 (m, 1H), 0.20-0.30 (m, 2H), −0.20-0.00 (m, 1H). |
| 162 | 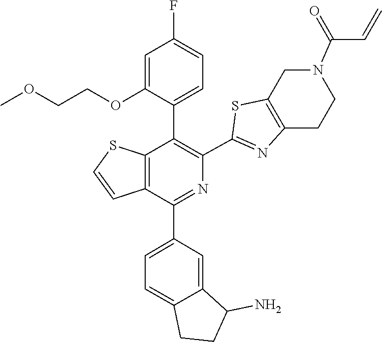 [SMILES: C=CC(=O)N1CCc2nc(-c3nc(-c4ccc5c(c4)C(N)CC5)c4ccsc4c3-c3ccc(F)cc3OCCOC)sc2C1] | LCMS m/z [M + 1]: 627.2 ¹H NMR (400 MHz, CD₃OD) δ8.13 (s, 1H), 8.01 (d, J = 8.2 Hz, 1H), 7.83 (d, J = 5.5 Hz, 1H), 7.75 (d, J = 5.6 Hz, 1H), 7.60 (d, J = 7.9 Hz, 1H), 7.32-7.41 (m, 1H), 6.73-6.95 (m, 3H), 6.25 (d, J = 16.8 Hz, 1H), 5.80 (d, J = 10.6 Hz, 1H), 4.91-4.98 (m, 1H), 4.01- 4.09 (m, 1H), 3.87-3.93 (m, 3H), 3.25-3.35 (m, 5H), 3.09-3.19 (m, 1H), 3.01 (s, 3H), 2.67-2.79 (m, 3H), 2.15-2.23 (m, 1H). |
| 163 | 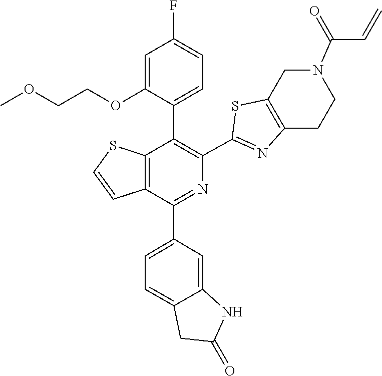 [SMILES: C=CC(=O)N1CCc2nc(-c3nc(-c4ccc5c(c4)NC(=O)C5)c4ccsc4c3-c3ccc(F)cc3OCCOC)sc2C1] | LCMS m/z [M + 1]: 627.2 ¹H NMR (400 MHz, CD₃OD) δ 7.72-7.80 (m, 2H), 7.57-7.62 (m, 1H),7.42-7.55 (m, 2H), 7.32-7.38 (m, 1H), 6.75-6.93 (m, 3H), 6.22-6.26 (m, 1H), 5.78- 5.81 (m, 1H), 4.02-4.03 (m, 1H),3.88-3.90 (m, 3H), 3.66 (s, 3H), 3.00 (s, 3H), 2.71-2.72 (m, 2H). |
| 164 | 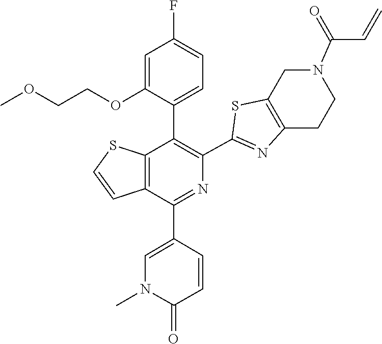 [SMILES: C=CC(=O)N1CCc2nc(-c3nc(-c4ccc(=O)n(C)c4)c4ccsc4c3-c3ccc(F)cc3OCCOC)sc2C1] | LCMS m/z [M + 1]: 603.2 ¹H NMR (400 MHz, CD₃OD) δ 8.41 (d, J = 2.1 Hz, 1H), 8.25 (dd, J = 9.4, 2.4 Hz, 1H), 7.86 (dd, J = 19.8, 5.5 Hz, 2H), 7.36 (t, J = 7.4 Hz, 1H), 6.75-6.97 (m, 4H), 6.24 (d, J = 16.6 Hz, 1H), 5.80 (d, J = 10.1 Hz, 1H), 3.85-4.06 (m, 4H), 3.75 (s, 3H), 3.00 (s, 3H), 2.78-2.83 (m, 2H). |
| 165 | 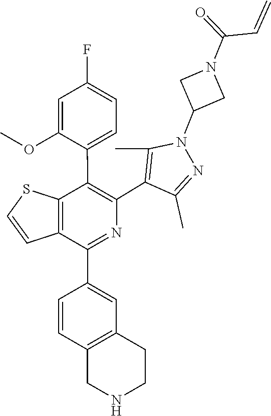 [SMILES: C=CC(=O)N1CC(n2nc(C)c(-c3nc(-c4ccc5c(c4)CCNC5)c4ccsc4c3-c3ccc(F)cc3OC)c2C)C1] | LCMS m/z [M + 1]: 594.4 ¹H NMR (400 MHz, CD₃OD) δ 7.98 (d, J-5.6 Hz, 1H), 7.77-7.80 (m, 3H), 7.52 (d, J = 7.7 Hz, 1H), 7.32 (s, 1H), 6.92 (d, J = 11.4 Hz, 1H), 6.80 (t, J = 8.2 Hz, 1H), 6.24-6.40 (m, 2H), 5.77 (dd, J = 10.0, 1.9 Hz, 1H), 5.18-5.19 (m, 1H), 4.69 (t, J = 8.8 Hz, 1H), 4.58- 4.61 (m, 1H), 4.52 (s, 2H), 4.44 (t, J = 9.4 Hz, 1H), 4.26-4.30 (m, 1H), 3.67 (s, 3H), 3.60 (t, J = 6.3 Hz, 2H), 3.28 (d, J = 6.4 Hz, 2H), 2.11 (s, 3H), 2.01 (s, 3H). |
| 166 | 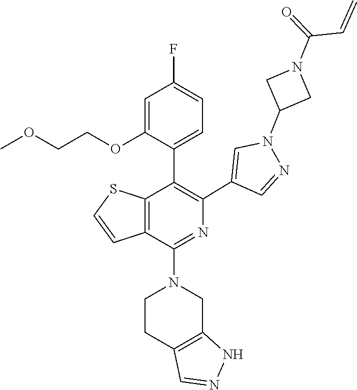 [SMILES: C=CC(=O)N1CC(n2cc(-c3nc(N4CCc5cn[nH]c5C4)c4ccsc4c3-c3ccc(F)cc3OCCOC)cn2)C1] | LCMS m/z [M + 1]: 600.2 ¹H NMR (400 MHz, CD₃OD) δ 7.73-7.77 (m, 3H), 7.63 (s, 1H), 7.53 (d, J = 3.6 Hz, 1H), 7.25 (dd, J = 8.3, 6.8 Hz, 1H), 7.01 (dd, J = 11.0, 2.0 Hz, 1H), 6.85 (dt, J = 8.3, 2.3 Hz, 1H), 6.25-6.39 (m, 2H), 5.77 (dd, J = 10.0, 2.2 Hz, 1H), 5.21-5.27 (m, 1H), 4.98 (s, 2H), 4.74 (t, J = 9.0 Hz, 1H), 4.47-4.55 (m, 2H), 4.27-4.31 (m, 1H), 4.11-4.14 (m, 2H), 3.98-4.05 (m, 3H), 3.42 (t, J = 4.5 Hz, 2H), 3.08 (s, 3H), 3.03 (t, J = 5.3 Hz, 2H). |
| 167 | 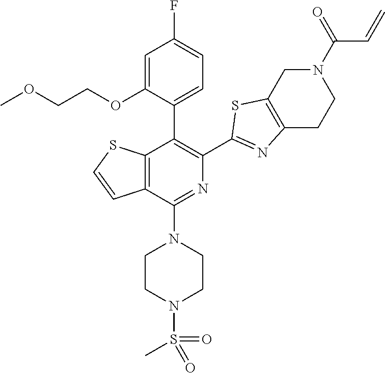 [SMILES: C=CC(=O)N1CCc2nc(-c3nc(N4CCN(S(C)(=O)=O)CC4)c4ccsc4c3-c3ccc(F)cc3OCCOC)sc2C1] | LCMS m/z [M + 1]: 658.2 ¹H NMR (400 MHz, CD₃OD) δ 7.73 (d, J = 5.5 Hz, 1H), 7.66 (d, J = 5.6 Hz, 1H), 7.29 (dd, J = 8.3, 6.8 Hz, 1H), 6.72-6.96 (m, 3H), 6.23 (d, J = 16.6 Hz, 1H), 5.78 (d, J = 10.8 Hz, 1H), 3.97-4.05 (m, 1H), 3.84- 3.96 (m, 3H), 3.76-3.84 (m, 4H), 3.50 (t, J = 4.9 Hz, 4H), 3.32-3.37 (m, 1H), 3.0 (s, 3H), 2.92 (s, 3H), 2.68-2.79 (m, 2H). |
| 168 | 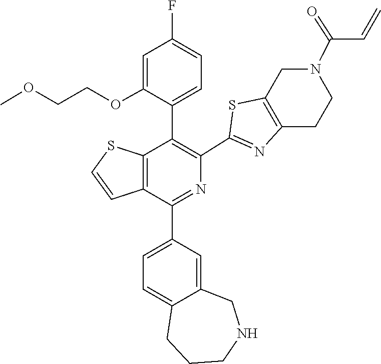 [SMILES: C=CC(=O)N1CCc2nc(-c3nc(-c4ccc5c(c4)CNCCC5)c4ccsc4c3-c3ccc(F)cc3OCCOC)sc2C1] | LCMS m/z [M + 1]: 641.4 ¹H NMR (400 MHz, CD₃OD) δ 8.02 (s, 1H), 7.96 (dd, J = 7.7, 1.4 Hz, 1H), 7.84 (d, J = 5.5 Hz, 1H), 7.75 (d, J = 5.6 Hz, 1H), 7.54 (d, J = 7.8 Hz, 1H), 7.36 (dd, J = 6.8, 8.3 Hz, 1H), 6.82-6.95 (m, 3H), 6.24 (d, J = 16.6 Hz, 1H), 5.80 (d, J = 10.4 Hz, 1H), 4.55 (s, 2H), 4.01-4.05 (m, 1H), 3.85-3.92 (m, 3H), 3.56 (t, J = 5.2 Hz, 2H), 3.30-3.36 (m, 3H), 3.20-3.23 (m, 2H), 3.00 (s, 3H), 2.73 (s, 2H), 2.10 (s, 2H). |
| 169 | 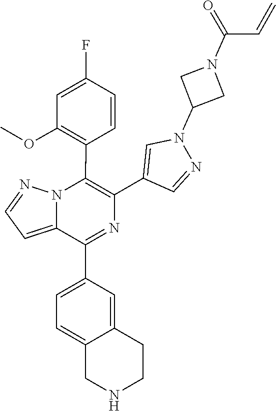 [SMILES: C=CC(=O)N1CC(n2cc(-c3nc(-c4ccc5c(c4)CCNC5)c4ccnn4c3-c3ccc(F)cc3OC)cn2)C1] | LCMS m/z [M + 1]: 550.2 ¹H NMR (400 MHz, CD₃OD) δ 8.06-8.10 (m, 2H), 8.02 (d, J = 2.4 Hz, 1H), 7.67 (s, 1H), 7.48 (d, J = 8.0 Hz, 1H), 7.38 (s, 1H), 7.32-7.36 (m, 1H), 7.15 (d, J = 2.4 Hz, 1H), 7.10(dd, J = 11.2, 2.4 Hz, 1H), 6.90- 6.96 (m, 1H), 6.24-6.40 (m, 2H), 5.77(dd, J = 10.0, 2.2 Hz, 1H), 5.20-5.28 (m, 1H), 4.73 (t, J = 8.8 Hz, 1H), 4.45-4.60 (m, 4H), 4.29-4.35 (m, 1H), 3.71 (s, 3H), 3.61 (t, J = 6.4 Hz, 2H). |
| 170 | 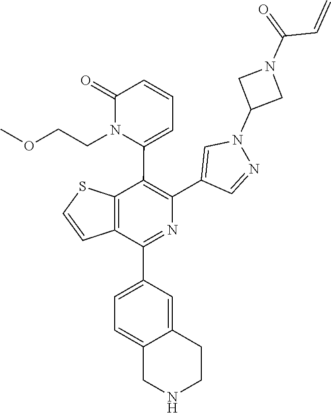 [SMILES: C=CC(=O)N1CC(n2cc(-c3nc(-c4ccc5c(c4)CCNC5)c4ccsc4c3-c3cccc(=O)n3CCOC)cn2)C1] | LCMS m/z [M + 1]: 593.2 ¹H NMR (400 MHz, CD₃OD) δ 7.82-7.87 (m, 4H), 7.79 (d, J = 8.0 Hz, 1H), 7.67 (d, J = 5.6 Hz, 1H), 7.48- 7.50 (m, 2H), 7.12 (d, J = 7.2 Hz, 1H), 6.96 (d, J = 8.0 Hz, 1H), 6.27-6.41 (m, 2H), 5.79 (dd, J = 10.0, 2.0 Hz, 1H), 5.25-5.32 (m, 1H), 4.76 (t, J = 8.8 Hz, 1H), 4.59-4.62 (m, 1H), 4.45-4.49 (m, 5H), 4.33-4.37 (m, 1H), 3.73 (t, J = 4.8 Hz, 2H), 3.62 (t, J = 6.4 Hz, 2H), 3.42 (s, 3H), 3.29-3.33 (m, 2H). |
| 171 | 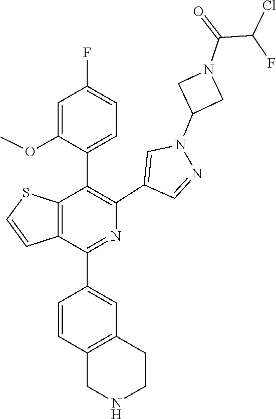 [SMILES: COc1cc(F)ccc1-c1c(-c2cnn(C3CN(C(=O)C(F)Cl)C3)c2)nc(-c2ccc3c(c2)CCNC3)c2ccsc12] | LCMS m/z [M + 1]: 606.2 ¹H NMR (400 MHz, CD₃OD) δ 7.84-7.88 (m, 3H), 7.77-7.81 (m, 1H), 7.67 (d, J = 5.6 Hz, 1H), 7.49-7.54 (m, 2H), 7.34 (t, J = 8.0 Hz, 1H), 7.05 (dd, J = 11.2, 2.4 Hz, 1H), 6.90-6.94 (m, 1H), 6.61-6.74 (m, 1H), 5.26-5.35 (m, 1H), 4.78-4.85 (m, 1H), 4.61-4.69 (m, 1H), 4.50-4.57 (m, 3H), 4.33-4.38 (m, 1H), 3.67 (s, 3H), 3.61 (t, J = 6.4 Hz, 2H), 3.28-3.31 (m, 2H). |
| 172 | 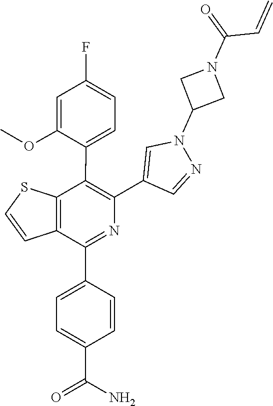 [SMILES: C=CC(=O)N1CC(n2cc(-c3nc(-c4ccc(C(N)=O)cc4)c4ccsc4c3-c3ccc(F)cc3OC)cn2)C1] | LCMS m/z [M + 1]: 554.4 ¹H NMR (400 MHz, DMSO-d6) δ 8.12-8.03 (m, 5H), 7.91(s, 1H), 7.83 (d, J = 5.6 Hz, 1H), 7.68 (d, J = 5.2 Hz, 1H), 7.48 (brs, 1H), 7.35 (t, J = 7.2 Hz, 1H), 7.22 (d, J = 9.6 Hz, 1H), 7.12 (s, 1H), 7.01 (t, J = 6.4 Hz, 1H), 6.33 (dd, J = 16.8, 10.0 Hz 1H), 6.12 (d, J = 15.6 Hz, 1H), 5.69 (d, J = 10.0 Hz 1H), 5.29 (m, 1H), 4.63 (t, J = 8.4 Hz, 1H), 4.41-4.31 (m, 2H), 4.16-4.12 (m, 1H), 3.65 (s, 3H). |
| 173 | 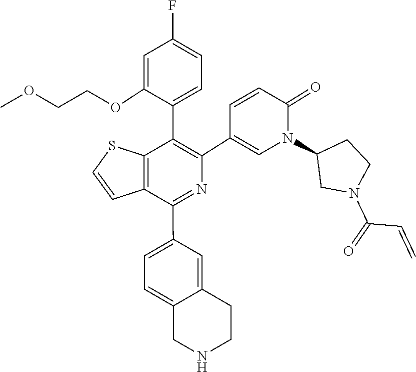 [SMILES: C=CC(=O)N1CC[C@H](n2cc(-c3nc(-c4ccc5c(c4)CCNC5)c4ccsc4c3-c3ccc(F)cc3OCCOC)ccc2=O)C1] | LCMS m/z [M + 1]: 651.2 ¹H NMR (400 MHz, CD₃OD) δ 8.26-8.31 (m, 1H), 7.96 (t, J = 5.9 Hz, 1H), 7.85-7.90 (m, 2H), 7.77-7.83 (m, 1H), 7.72-7.75 (m, 1H), 7.50-7.54 (m, 1H), 7.32- 7.83 (m, 1H), 6.94-6.99 (m, 1H), 6.81-6.87 (m, 1H), 6.73-6.78 (m, 1H), 6.48-6.68 (m, 1H), 6.23-6.30 (m, 1H), 5.70-5.78 (m, 1H), 5.56-5.65 (m, 1H), 4.52 (s, 2H), 4.01-4.08 (m, 1H), 3.92-3.98 (m, 1H), 3.71-3.88 (m, 3H), 3.61 (t, J = 6.3 Hz, 2H), 3.45-3.50 (m, 2H), 3.14-3.17 (m, 3H), 2.17-2.34 (m, 2H). |
| 173a | 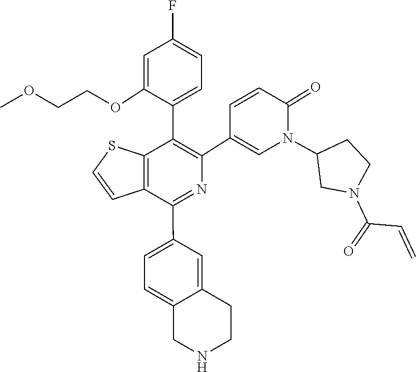 [SMILES: C=CC(=O)N1CCC(n2cc(-c3nc(-c4ccc5c(c4)CCNC5)c4ccsc4c3-c3ccc(F)cc3OCCOC)ccc2=O)C1] | |
| 174 | 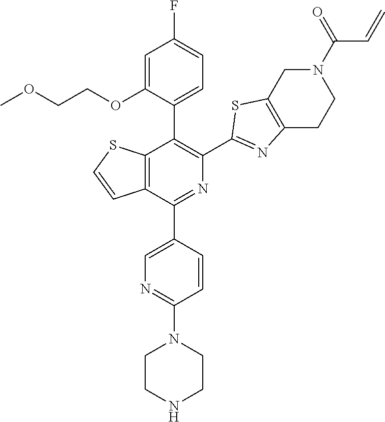 [SMILES: C=CC(=O)N1CCc2nc(-c3nc(-c4ccc(N5CCNCC5)nc4)c4ccsc4c3-c3ccc(F)cc3OCCOC)sc2C1] | LCMS m/z [M + 1]: 657.3 ¹H NMR (400 MHz, CD₃OD) δ 8.83 (d, J = 2.1 Hz, 1H), 8.31 (dd, J = 8.7, 1.9 Hz, 1H), 7.84 (d, J = 5.4 Hz, 1H), 7.76 (d, J = 5.6 Hz, 1H), 7.36 (t, J = 6.9 Hz, 1H), 7.16 (d, J = 8.9 Hz, 1H), 6.82-6.94 (m, 3H), 6.25 (d, J = 17.4 Hz, 1H), 5.80 (d, J = 9.5 Hz, 1H), 3.82-4.07 (m, 8H), 3.39 (t, J = 5.2 Hz, 4H), 3.00 (s, 3H), 2.73 (s, 2H). |
| 175 | 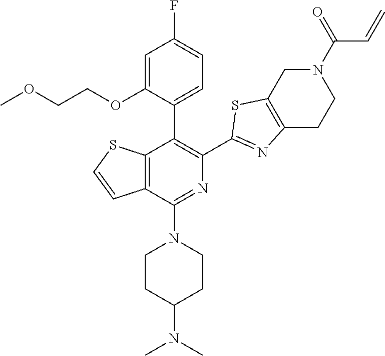 [SMILES: C=CC(=O)N1CCc2nc(-c3nc(N4CCC(N(C)C)CC4)c4ccsc4c3-c3ccc(F)cc3OCCOC)sc2C1] | LCMS m/z [M + 1]: 622.3 ¹H NMR (400 MHz, CD₃OD) δ 7.67 (d, J = 5.5 Hz, 1H), 7.59 (d, J = 5.6 Hz, 1H), 7.25 (dd, J = 8.2, 6.8 Hz, 1H), 6.72-6.94 (m, 3H), 6.17-6.30 (m, 1H), 5.75- 5.83 (m, 1H), 4.75-4.83 (m, 1H), 4.34-4.48 (m, 2H), 3.96-4.04 (m, 1H), 3.79-3.94 (m, 3H), 3.45-3.59 (m, 1H), 3.1-3.29 (m, 3H), 3.0 (s, 3H), 2.93 (s, 6H), 2.59- 2.73 (m, 2H), 2.16-2.3 (m, 2H), 1.93-2.13 (m, 2H). |
| 176 | 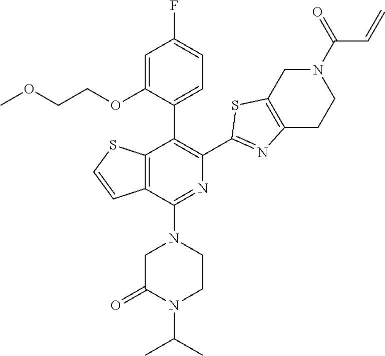 [SMILES: C=CC(=O)N1CCc2nc(-c3nc(N4CCN(C(C)C)C(=O)C4)c4ccsc4c3-c3ccc(F)cc3OCCOC)sc2C1] | LCMS m/z [M + 1]: 636.3 ¹H NMR (400 MHz, CD₃OD) δ 7.79 (d, J = 5.6 Hz, 1H), 7.76 (d, J = 5.6 Hz, 1H), 7.31 (dd, J = 8.4, 6.7 Hz, 1H), 6.97 (dd, J = 11.1, 2.2 Hz, 1H), 6.72-6.92 (m, 2H), 6.18-6.29 (m, 1H), 5.74-5.84 (m, 1H), 4.76- 4.87 (m, 3H), 4.43 (s, 2H), 3.97-4.1 (m, 3H), 3.86- 3.96 (m, 3H), 3.59 (t, J = 5.3 Hz, 2H), 3.32-3.37 (m, 1H), 2.99 (s, 3H), 2.71-2.87 (m, 2H), 1.21 (d, J = 6.8 Hz, 6H). |
| 177 | 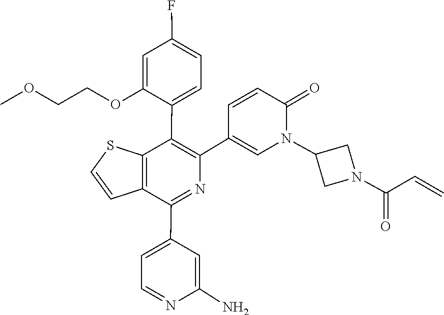 [SMILES: C=CC(=O)N1CC(n2cc(-c3nc(-c4ccnc(N)c4)c4ccsc4c3-c3ccc(F)cc3OCCOC)ccc2=O)C1] | LCMS m/z [M + 1]: 598.3 ¹H NMR (400 MHz, CD₃OD) δ 8.02 (d, J = 7.6 Hz, 1H), 7.91-7.96 (m, 2H), 7.61-7.81 (m, 2H), 7.60 (d, J = 1.2 Hz, 1H), 7.50-7.55 (m, 1H), 7.30-7.40 (m, 1H), 6.80-7.10 (m, 2H), 6.53-6.56 (m, 1H), 6.30-6.35 (m, 2H), 5.80-5.85 (m, 1H), 5.40-5.55 (m, 1H), 4.66-4.86 (m, 1H), 4.30-4.50 (m, 1H), 3.80-4.20 (m, 5H), 3.30- 3.50 (m, 2H), 3.08 (s, 3H). |
| 178 | 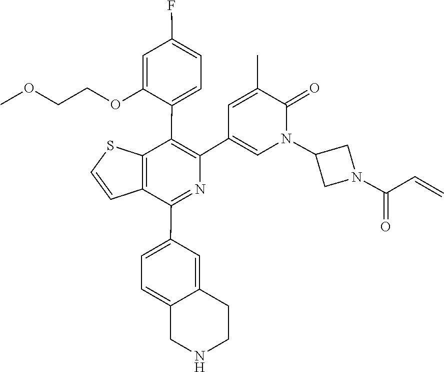 [SMILES: C=CC(=O)N1CC(n2cc(-c3nc(-c4ccc5c(c4)CCNC5)c4ccsc4c3-c3ccc(F)cc3OCCOC)cc(C)c2=O)C1] | LCMS m/z [M + 1]: 651.3 ¹H NMR (400 MHz, CD₃OD) δ 8.01 (t, J = 2.4 Hz, 1H), 7.82-7.85 (m, 3H), 7.66-7.68 (m, 2H), 7.47 (d, J = 8.0 Hz, 1H), 7.28-7.33 (m, 1H), 6.94 (dd, J = 11.1, 2.3 Hz, 1H), 6.81 (t, J = 8.1 Hz, 1H), 6.22-6.38 (m, 2H), 5.74 (dd, J = 10.1, 2.1 Hz, 1H), 5.38-5.42 (m, 1H), 4.68-4.72 (m, 1H), 4.50 (s, 2H), 4.43-4.47 (m, 1H), 4.25-4.28 (m, 1H), 3.99-4.05 (m, 2H), 3.88-3.93 (m, 1H), 3.59 (t, J = 6.4 Hz, 2H), 3.45 (t, J = 4.6 Hz, 2H), 3.28 (t, J = 6.0 Hz, 2H), 3.13 (s, 3H), 2.18 (s, 3H) |
| 179 | 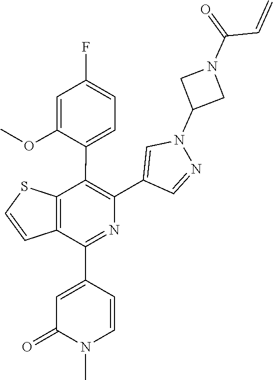 [SMILES: C=CC(=O)N1CC(n2cc(-c3nc(-c4ccn(C)c(=O)c4)c4ccsc4c3-c3ccc(F)cc3OC)cn2)C1] | LCMS m/z [M + 1]: 542.3 ¹H NMR (400 MHz, DMSO-d6) δ 7.90-7.85 (m, 3H), 7.67(d, J = 5.6 Hz, 1H), 7.34 (t, J = 7.6 Hz, 1H), 7.21 (d, J = 10.0 Hz, 1H), 7.10 (s, 1H), 7.01 (t, J = 6.8 Hz, 1H), 6.88 (s, 1H), 6.78 (d, J = 6.8 Hz, 1H), 6.33 (dd, J = 16.8, 10.0 Hz, 1H), 6.12 (d, J = 16.0 Hz, 1H), 5.69 (d, J = 10.4 Hz, 1H), 5.29 (m, 1H), 4.63 (m, 1H), 4.40-4.31 (m, 2H), 4.16-4.09 (m, 1H), 3.64 (s, 3H), 3.54 (s, 3H). |
| 180 | 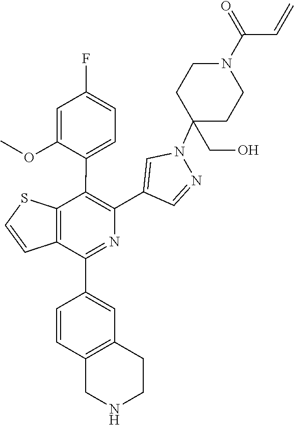 [SMILES: C=CC(=O)N1CCC(CO)(n2cc(-c3nc(-c4ccc5c(c4)CCNC5)c4ccsc4c3-c3ccc(F)cc3OC)cn2)CC1] | LCMS m/z [M + 1]: 624.3 ¹H NMR (400 MHz, CD₃OD) δ 7.83-7.89 (m, 2H), 7.75-7.81 (m, 1H), 7.63 (d, J = 5.4 Hz, 2H), 7.59 (s, 1H), 7.49 (d, J = 7.8 Hz, 1H), 7.32-7.40 (m, 1H), 7.04 (dd, J = 11.0, 2.2 Hz, 1H), 6.89-6.96 (m, 1H), 6.76 (dd, J = 16.8, 10.7 Hz, 1H), 6.18 (dd, J = 16.8, 1.8 Hz, 1H), 5.74 (dd, J = 10.6, 1.8 Hz, 1H), 4.52 (s, 2H), 4.27 (d, J = 13.4 Hz, 1H), 3.93 (d, J = 13.5 Hz, 1H), 3.66 (s, 3H), 3.61 (t, J = 6.4 Hz, 2H), 3.55 (s, 2H), 2.97-3.10 (m, 1H), 2.68-2.80 (m, 1H), 2.31-2.48 (m, 2H), 1.80-1.95 (m, 2H). |
| 181 | 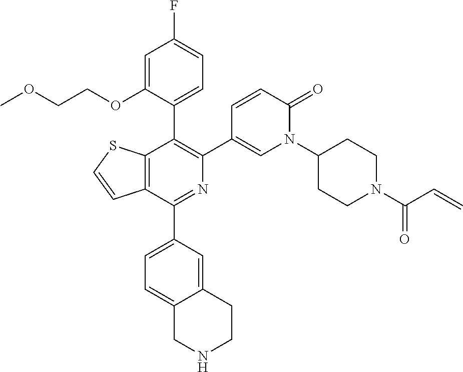 [SMILES: C=CC(=O)N1CCC(n2cc(-c3nc(-c4ccc5c(c4)CCNC5)c4ccsc4c3-c3ccc(F)cc3OCCOC)ccc2=O)CC1] | LCMS m/z [M + 1]: 665.2 ¹H NMR (400 MHz, CD₃OD) δ 8.21-8.25 (m, 1H), 7.78-7.94 (m, 4H), 7.66-7.72 (m, 1H), 7.46-7.51 (m, 1H), 7.30-7.36 (m, 1H), 6.92-6.99 (m, 1H), 6.71-6.86 (m, 3H), 6.19 (dd, J = 16.8, 1.9 Hz, 1H), 5.74 (dd, J = 10.7, 1.9 Hz, 1H), 5.22-5.30 (m, 1H), 4.51 (s, 2H), 4.00-4.08 (m, 1H), 3.84-3.98 (m, 3H), 3.49-3.63 (m, 4H), 3.44-3.49 (m, 2H), 3.11-3.15 (m, 3H), 1.97-2.09 (m, 2H), 1.68-1.81 (m, 2H). |
| 182 | 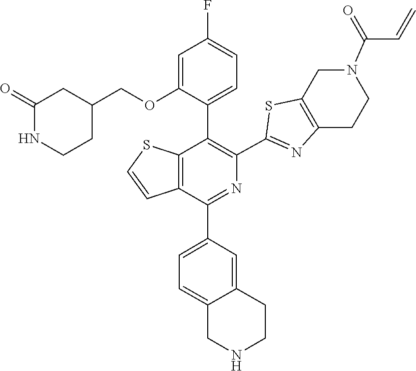 [SMILES: C=CC(=O)N1CCc2nc(-c3nc(-c4ccc5c(c4)CCNC5)c4ccsc4c3-c3ccc(F)cc3OCC3CCNC(=O)C3)sc2C1] | LCMS m/z [M + 1]: 680.3 ¹H NMR (400 MHz, CD₃OD) δ 7.88-7.92 (m, 2H), 7.83 (d, J = 5.6 Hz, 1H), 7.68-7.74 (m, 1H), 7.46 (d, J = 7.8 Hz, 1H), 7.33-7.41 (m, 1H), 6.82-6.97 (m, 3H), 6.25 (d, J = 17.2 Hz, 1H), 5.76-5.81 (m, 1H), 4.50 (s, 2H), 3.72-3.99 (m, 4H), 3.61 (t, J = 6.3 Hz, 2H), 3.21-3.35(m, 4H), 3.00-3.04 (m, 2H), 2.71-2.75 (m, 2H), 1.91-1.99 (m, 2H), 1.45-1.75 (m, 2H), 1.08- 1.21 (m, 1H). |
| 183 | 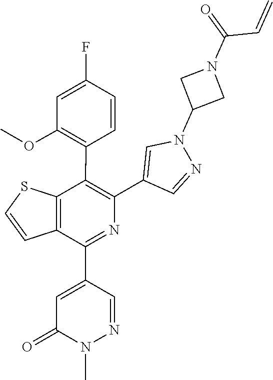 [SMILES: C=CC(=O)N1CC(n2cc(-c3nc(-c4cnn(C)c(=O)c4)c4ccsc4c3-c3ccc(F)cc3OC)cn2)C1] | LCMS m/z [M + 1]: 543.4 ¹H NMR (400 MHz, CD₃OD) δ 8.50 (s, 1H), 7.77- 7.75 (m, 2H), 7.71 (d, J = 5.6 Hz, 1H), 7.40 (s, 1H), 7.37 (s, 1H), 7.31-7.28 (m, 1H), 7.05 (dd, J = 11.2, 2.4 Hz, 1H), 6.93 (td, J = 8.4, 2.4 Hz, 1H), 6.39-6.24 (m, 2H), 5.76 (dd, J = 9.6, 2.0 Hz, 1H), 5.27-5.20 (m, 1H), 4.72 (t, J = 9.2 Hz, 1H), 4.58-4.54 (m, 1H), 4.48 (t, J = 10.4 Hz, 1H), 4.33-4.29 (m, 1H), 3.88 (s, 3H), 3.65 (s, 3H) |
| 184 | 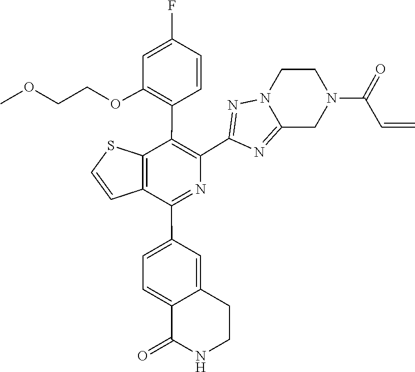 [SMILES: C=CC(=O)N1CCn2nc(-c3nc(-c4ccc5c(c4)CCNC5=O)c4ccsc4c3-c3ccc(F)cc3OCCOC)nc2C1] | LCMS m/z [M + 1]: 625.2 ¹H NMR (400 MHz, CD₃OD) δ 8.15 (d, J = 7.9 Hz, 1H), 7.91-7.98 (m, 3H), 7.77 (d, J = 5.6 Hz, 1H), 7.37-7.43 (m, 1H), 6.94 (dd, J = 11.1, 2.2 Hz, 1H), 6.77-6.88 (m, 2H), 6.30 (d, J = 16.6 Hz, 1H), 5.85 (d, J = 10.7 Hz, 1H), 4.90-5.03 (m, 2H), 4.15 (s, 4H), 3.99-4.07 (m, 1H), 3.86-3.94 (m, 1H), 3.59 (t, J = 6.6 Hz, 2H), 3.40 (t, J = 4.6 Hz, 2H), 3.13 (t, J = 6.6 Hz, 2H), 3.06 (s, 3H). |
| 185 | 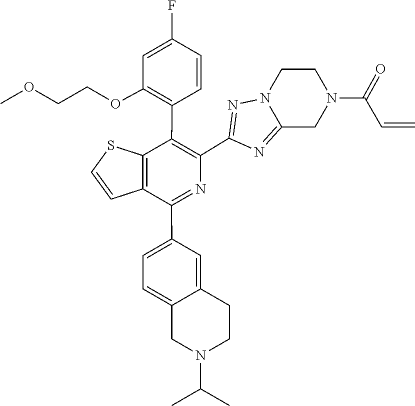 [SMILES: C=CC(=O)N1CCn2nc(-c3nc(-c4ccc5c(c4)CCN(C(C)C)C5)c4ccsc4c3-c3ccc(F)cc3OCCOC)nc2C1] | LCMS m/z [M + 1]: 653.3 ¹H NMR (400 MHz, CD₃OD) δ 7.95 (s, 1H), 7.92 (d, J = 8.08 Hz, 1H), 7.86 (d, J = 5.6 Hz, 1H), 7.70 (d, J = 5.6 Hz, 1H), 7.48 (d, J = 8.0 Hz, 1H), 7.35 (t, J = 7.4 Hz, 1H), 6.79-6.92 (m, 3H), 6.30(dd, J = 16.8, 1.4 Hz, 1H), 5.85 (dd, J = 10.6, 1.8 Hz, 1H), 4.91- 5.00 (m, 2H), 4.59 (s, 2H), 4.11-4.17 (m, 4H), 3.98- 4.04 (m, 1H), 3.77-3.88 (m, 3H), 3.32-3.55 (m, 5H), 3.06 (s, 3H), 1.51 (d, J = 6.6 Hz, 6H). |
| 186 | 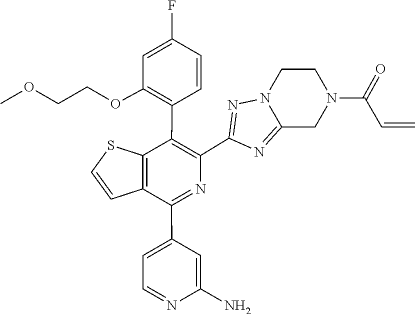 [SMILES: C=CC(=O)N1CCn2nc(-c3nc(-c4ccnc(N)c4)c4ccsc4c3-c3ccc(F)cc3OCCOC)nc2C1] | LCMS m/z [M + 1]: 572.2 ¹H NMR (400 MHz, CD₃OD) δ 8.02 (d, J = 6.6 Hz, 1H), 7.98 (d, J = 5.6 Hz, 1H), 7.81 (d, J = 5.6 Hz, 1H), 7.62 (s, 1H), 7.57 (d, J = 6.8 Hz, 1H), 7.35 (dd, J = 8.4, 6.6 Hz, 1H), 6.75-6.93 (m, 3H), 6.28-6.33 (m, 1H), 5.85 (dd, J = 10.6, 1.7 Hz, 1H), 4.91-4.95 (m, 2H), 4.14-4.20 (m, 4H), 3.99-4.05 (m, 1H), 3.84-3.89 (m, 1H), 3.38 (t, J = 4.6 Hz, 2H), 3.05 (s, 3H). |
| 187 | 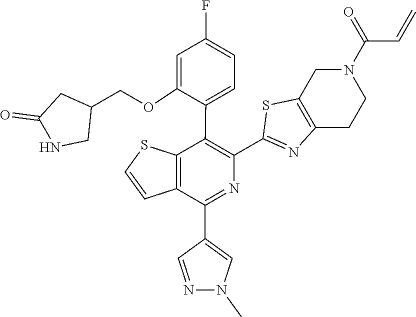 [SMILES: C=CC(=O)N1CCc2nc(-c3nc(-c4cnn(C)c4)c4ccsc4c3-c3ccc(F)cc3OCC3CNC(=O)C3)sc2C1] | LCMS m/z [M + 1]: 615.2 ¹H NMR (400 MHz, CD₃OD) δ 8.55 (s, 1H), 8.32 (s, 1H), 7.96-7.99 (m, 2H), 7.38-7.42 (m, 1H), 7.01-7.04 (m, 1H), 6.74-6.93 (m, 2H), 6.20-6.28 (m, 1H), 5.80- 5.82 (m, 1H), 4.08 (s, 3H), 3.86-3.95 (m, 4H), 3.14- 3.18 (m, 1H), 2.82-2.86 (m, 2H), 2.65-2.73(m, 1H), 2.54-2.58 (m, 1H), 2.03-2.15 (m, 1H), 1.57-1.76 (m, 1H). |
| 188 | 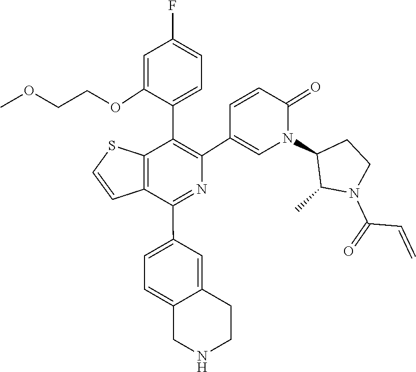 [SMILES: C=CC(=O)N1CC[C@H](n2cc(-c3nc(-c4ccc5c(c4)CCNC5)c4ccsc4c3-c3ccc(F)cc3OCCOC)ccc2=O)[C@H]1C] | LCMS m/z [M + 1]: 665.3 ¹H NMR (400 MHz, CD₃OD) δ 8.28 (d, J = 12.0 Hz, 1H), 7.85-7.91 (m, 3H), 7.70-7.80 (m, 2H), 7.50 (d, J = 8.5 Hz, 1H), 7.32-7.38 (m, 1H), 6.93-6.97 (m, 1H), 6.80-6.86 (m, 1H), 6.50-6.73 (m, 2H), 6.27 (t, J = 14.8 Hz, 1H), 5.71-5.75 (m, 1H), 5.30-5.33 (m, 1H), 5.21 (d, J = 4.1 Hz, 1H), 4.51 (s, 2H), 4.24-4.28 (m, 1H), 4.01-4.04 (m, 1H), 3.85-3.95 (m, 1H), 3.67-3.78 (m, 1H), 3.60 (t, J = 6.4 Hz, 2H), 3.45-3.47 (m, 2H), 3.13-3.14 (m, 3H), 2.35-2.47 (m, 1H), 2.16-2.25 (m, 1H), 1.28-1.32 (m, 5H), 0.85-0.96 (m, 2H). |
| 189 | 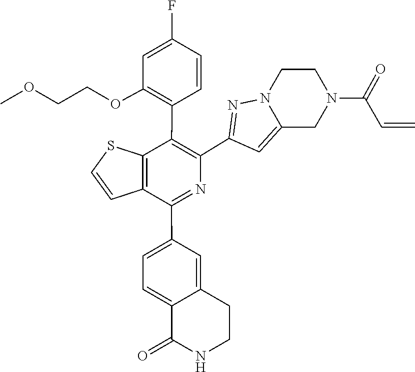 [SMILES: C=CC(=O)N1CCn2nc(-c3nc(-c4ccc5c(c4)CCNC5=O)c4ccsc4c3-c3ccc(F)cc3OCCOC)cc2C1] | LCMS m/z [M + 1]: 624.3 ¹H NMR (400 MHz, CD₃OD) δ 8.23 (d, J = 7.9 Hz, 1H), 8.09 (d, J = 5.6 Hz, 1H), 7.93-7.98 (m, 2H), 7.81 (d, J = 5.6 Hz, 1H), 7.41 (dd, J = 8.3, 6.6 Hz, 1H), 7.09 (dd, J = 11.0, 2.3 Hz, 1H), 6.70-6.98 (m, 2H), 6.28 (d, J = 15.7 Hz, 1H), 5.83 (s, 2H), 4.70-4.86 (m, 2H), 4.28 (s, 2H), 4.06-4.16 (m, 3H), 3.95-4.04 (m, 1H), 3.61 (t, J = 6.6 Hz, 2H), 3.42 (t, J = 4.5 Hz, 2H), 3.17 (t, J = 6.6 Hz, 2H), 3.09 (s, 3H). |
| 190 | 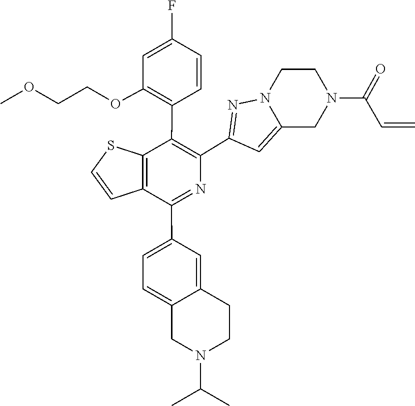 [SMILES: C=CC(=O)N1CCn2nc(-c3nc(-c4ccc5c(c4)CCN(C(C)C)C5)c4ccsc4c3-c3ccc(F)cc3OCCOC)cc2C1] | LCMS m/z [M + 1]: 652.2 ¹H NMR (400 MHz, CD₃OD) δ 7.87-7.98 (m, 3H), 7.67-7.74 (m, 1H), 7.50-7.57 (m, 1H), 7.31-7.39 (m, 1H), 6.99-7.07 (m, 1H), 6.75-6.92 (m, 2H), 6.28 (d, J = 15.5 Hz, 1H), 5.79-5.92 (m, 2H), 4.73-4.80 (m, 1H), 4.61 (s, 2H), 4.21-4.30 (m, 2H), 4.03-4.16 (m, 3H), 3.92-4.00 (m, 1H), 3.75-3.89 (m, 2H), 3.34-3.55 (m, 5H), 3.07 (s, 3H), 1.51 (d, J = 6.7 Hz, 6H). |
| 191 | 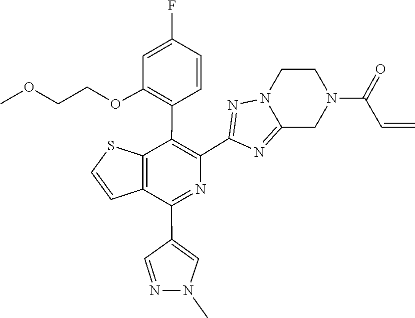 [SMILES: C=CC(=O)N1CCn2nc(-c3nc(-c4cnn(C)c4)c4ccsc4c3-c3ccc(F)cc3OCCOC)nc2C1] | LCMS m/z [M + 1]: 560.2 ¹H NMR (400 MHz, CD₃OD) δ 8.63 (s, 1H), 8.34 (s, 1H), 8.12 (d, J = 5.6 Hz, 1H), 8.06 (d, J = 5.6 Hz, 1H), 7.40 (dd, J = 8.4, 6.8 Hz, 1H), 6.96 (dd, J = 11.2, 2.4 Hz, 1H), 6.84-6.89 (m, 2H), 6.31 (dd, J = 16.8, 1.6 Hz, 1H), 5.86 (dd, J = 10.4, 1.6 Hz, 1H), 4.93- 5.08 (m, 2H), 4.15-4.20 (m, 4H), 4.09 (s, 3H), 4.02- 4.07 (m, 1H), 3.88-3.93 (m, 1H), 3.37 (t, J = 4.4 Hz, 2H), 3.05 (s, 3H). |
| 192 | 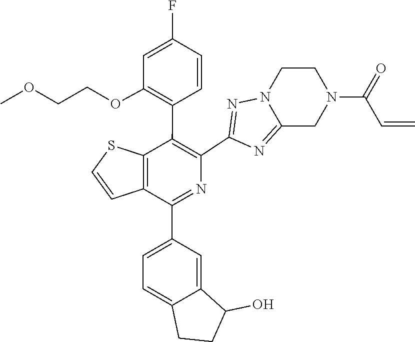 [SMILES: C=CC(=O)N1CCn2nc(-c3nc(-c4ccc5c(c4)C(O)CC5)c4ccsc4c3-c3ccc(F)cc3OCCOC)nc2C1] | LCMS m/z [M + 1]: 612.2 ¹H NMR (400 MHz, CD₃OD) δ 8.13 (dd, J = 5.6, 0.8 Hz, 1H), 7.96 (s, 1H), 7.84-7.88 (m, 2H), 7.57 (d, J = 7.6 Hz, 1H), 7.42-7.47 (m, 1H), 6.97-7.00 (m, 1H), 6.86-6.91 (m, 2H), 6.27-6.32 (m, 1H), 5.84 (d, J = 10.4 Hz, 1H), 5.33 (q, J = 6.4 Hz, 1H), 4.96-4.98 (m, 2H), 4.15-4.18 (m, 4H), 4.04-4.09 (m, 1H), 3.90-3.96 (m, 1H), 3.40 (t, J = 4.0 Hz, 2H), 3.15-3.23 (m, 1H), 2.93-3.01 (m, 1H), 2.52-2.61 (m, 1H), 2.00-2.09 (m, 1H). |
| 193 | 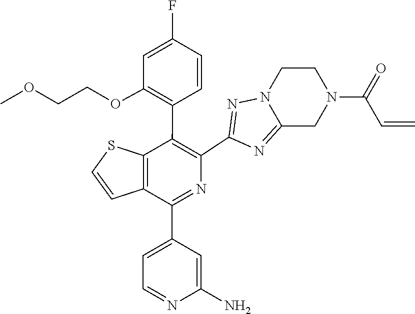 [SMILES: C=CC(=O)N1CCn2nc(-c3nc(-c4ccnc(N)c4)c4ccsc4c3-c3ccc(F)cc3OCCOC)nc2C1] | LCMS m/z [M + 1]: 571.2 ¹H NMR (400 MHz, CD₃OD) δ 8.01 (d, J = 6.7 Hz, 1H), 7.89 (d, J = 5.6 Hz, 1H), 7.77 (d, J = 5.6 Hz, 1H), 7.60 (s, 1H), 7.54 (dd, J = 6.7, 1.6 Hz, 1H), 7.31 (dd, J = 8.3, 6.6 Hz, 1H), 6.99 (dd, J = 11.1, 2.3 Hz, 1H), 6.80-6.91 (m, 2H), 6.28 (d, J = 16.7 Hz, 1H), 5.94 (d, J = 15.3 Hz, 1H), 5.82 (d, J = 10.4 Hz, 1H), 4.71-4.85 (m, 2H), 4.19 (s, 2H), 4.09-4.14 (m, 2H), 3.99-4.07 (m, 1H), 3.86-3.96 (m, 1H), 3.38 (t, J = 4.7 Hz, 2H), 3.04 (s, 3H). |
| 194 | 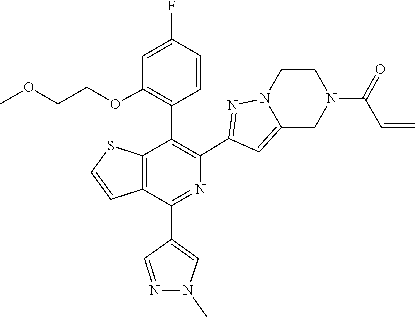 [SMILES: C=CC(=O)N1CCn2nc(-c3nc(-c4cnn(C)c4)c4ccsc4c3-c3ccc(F)cc3OCCOC)cc2C1] | LCMS m/z [M + 1]: 559.2 ¹H NMR (400 MHz, CD₃OD) δ 8.68 (s, 1H), 8.35 (s, 1H), 8.16 (d, J = 6.0 Hz, 1H), 8.09 (d, J = 5.6 Hz, 1H), 7.41 (dd, J = 8.4, 6.4 Hz, 1H), 7.10 (dd, J = 11.2, 2.4 Hz, 1H), 6.85-6.97 (m, 2H), 6.29 (d, J = 16.4 Hz, 1H), 5.81-5.85 (m, 2H), 4.7-4.86 (m, 2H), 4.35 (s, 2H), 4.14-4.17 (m, 2H), 4.11 (s, 3H), 4.06-4.11 (m, 1H), 3.98-4.03 (m, 1H), 3.40 (t, J = 4.4 Hz, 2H), 3.07 (s, 3H). |
| 195 | 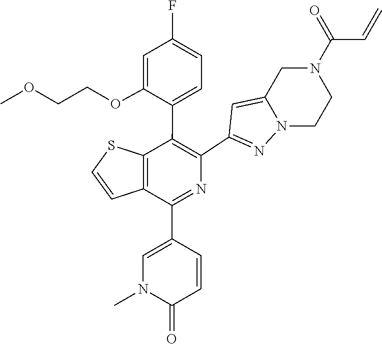 [SMILES: C=CC(=O)N1CCn2nc(-c3nc(-c4ccc(=O)n(C)c4)c4ccsc4c3-c3ccc(F)cc3OCCOC)cc2C1] | LCMS m/z [M + 1]: 586.2 ¹H NMR (400 MHz, CD₃OD) δ 8.54 (d, J = 2.4 Hz, 1H), 8.13 (dd, J = 9.6, 2.8 Hz, 1H), 8.06 (d, J = 5.6 Hz, 1H), 7.90 (d, J = 5.6 Hz, 1H), 7.37 (dd, J = 8.4, 6.8 Hz, 1H), 7.07 (dd, J = 10.8, 2.4 Hz, 1H), 6.90- 6.95 (m, 1H), 6.77 (d, J = 9.4 Hz, 2H), 6.28 (d, J = 15.6 Hz, 1H), 5.81-5.85 (m, 2H), 4.75-4.87 (m, 2H), 4.29 (s, 2H), 4.12-4.15 (m, 2H), 4.05-4.10 (m, 1H), 3.95-4.00 (m, 1H), 3.73 (s, 3H), 3.40 (t, J = 4.4 Hz, 2H), 3.08 (s, 3H). |
| 196 | 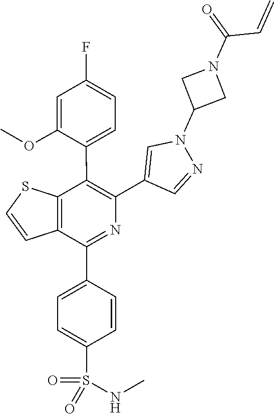 [SMILES: C=CC(=O)N1CC(n2cc(-c3nc(-c4ccc(S(=O)(=O)NC)cc4)c4ccsc4c3-c3ccc(F)cc3OC)cn2)C1] | LCMS m/z [M + 1]: 604.4 ¹H NMR (400 MHz, DMSO-d6) δ 8.20 (d, J = 8.4 Hz, 2H), 7.99-7.92 (m, 3H), 7.86 (d, J = 5.6 Hz, 1H), 7.71 (d, J = 5.6 Hz, 1H), 7.63-7.59 (m, 1H), 7.35 (t, J = 7.6 Hz, 1H), 7.22 (dd, J = 11.2, 2.4 Hz, 1H), 7.13 (s, 1H), 7.01 (td, J = 8.4, 2.4 Hz, 1H), 6.36-6.29 (dd, J = 16.8, 10.0 Hz 1H), 6.12 (dd, J = 16.8, 2.4 z, 1H), 5.69 (dd, J = 10.4, 2.0 Hz, 1H), 5.31-5.25 (m, 1H), 4.63 (t, J = 8.4 Hz, 1H), 4.42-4.31 (m, 2H), 4.14-4.12 (m, 1H), 3.65 (s, 3H), 2.51 (s, 3H). |
| 197 | 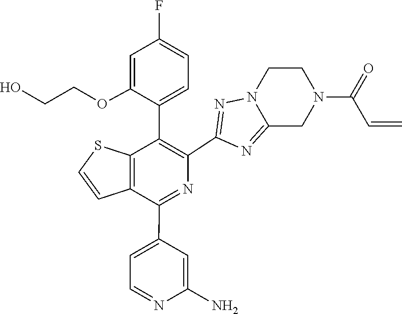 [SMILES: C=CC(=O)N1CCn2nc(-c3nc(-c4ccnc(N)c4)c4ccsc4c3-c3ccc(F)cc3OCCO)nc2C1] | LCMS m/z [M + 1]: 557.2 ¹H NMR (400 MHz, CD₃OD) δ 8.01 (d, J = 6.7 Hz, 1H), 7.89 (d, J = 5.6 Hz, 1H), 7.77 (d, J = 5.6 Hz, 1H), 7.60 (s, 1H), 7.54 (dd, J = 6.7, 1.6 Hz, 1H), 7.31 (dd, J = 8.4, 6.7 Hz, 1H), 6.98 (dd, J = 11.1, 2.3 Hz, 1H), 6.83-6.90 (m, 2H), 6.25-6.30 (m, 1H), 5.96-6.00 (m, 1H), 5.82 (d, J = 10.1 Hz, 1H), 4.77 (s, 1H), 4.11- 4.19 (m, 4H), 3.95-4.00 (m, 1H), 3.83-3.88 (m, 1H), 3.48-3.57 (m, 2H). |
| 198 | 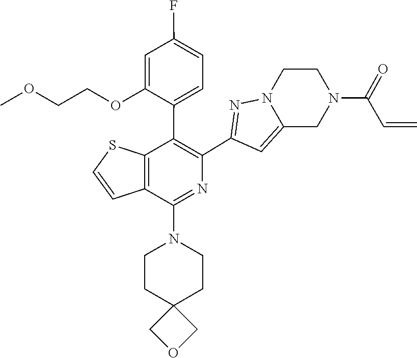 [SMILES: C=CC(=O)N1CCn2nc(-c3nc(N4CCC5(CC4)COC5)c4ccsc4c3-c3ccc(F)cc3OCCOC)cc2C1] | LCMS m/z [M + 1]: 604.3 ¹H NMR (400 MHz, CD₃OD) δ 7.91 (d, J = 5.7 Hz, 1H), 7.89 (d, J = 5.7 Hz, 1H), 7.26-7.35 (m, 1H), 7.09 (dd, J = 11.0, 2.2 Hz, 1H), 6.70-6.95 (m, 2H), 6.28 (d, J = 16.6 Hz, 1H), 5.75-5.88 (m, 1H), 5.58 (s, 1H), 4.66-4.85 (m, 2H), 4.60 (s, 4H), 4.31 (s, 2H), 4.11- 4.18 (m, 2H), 4.00-4.10 (m, 2H), 3.80-3.96 (m, 4H), 3.40 (t, J = 4.5 Hz, 2H), 3.07 (s, 3H), 2.21-2.30 (m, 4H). |
| 199 | 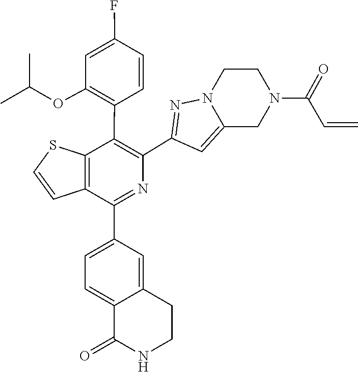 [SMILES: C=CC(=O)N1CCn2nc(-c3nc(-c4ccc5c(c4)CCNC5=O)c4ccsc4c3-c3ccc(F)cc3OC(C)C)cc2C1] | LCMS m/z [M + 1]: 608.3 ¹H NMR (400 MHz, CD₃OD) δ 8.23 (d, J = 7.9 Hz, 1H), 8.07 (d, J = 5.6 Hz, 1H), 7.98 (d, J = 10.5 Hz, 2H), 7.82 (d, J = 5.7 Hz, 1H), 7.41 (d, J = 7.4 Hz, 1H), 7.01 (dd, J = 11.4, 2.1 Hz, 1H), 6.73-6.92 (m, 2H), 6.27 (d, J = 16.6 Hz, 1H), 5.81 (s, 2H), 4.81 (m, 2H), 4.53-4.59 (m, 1H), 4.30 (s, 2H), 4.13 (t, J = 5.7 Hz, 2H), 3.61 (t, J = 6.6 Hz, 2H), 3.18 (t, J = 6.5 Hz, 2H), 1.10 (d, J = 6.0 Hz, 3H), 0.91 (d, J = 6.0 Hz, 3H). |
| 200 | 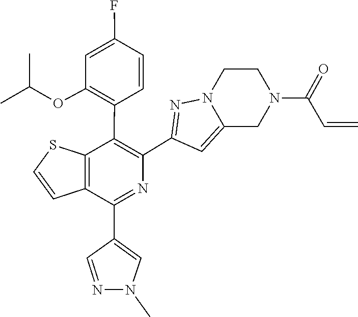 [SMILES: C=CC(=O)N1CCn2nc(-c3nc(-c4cnn(C)c4)c4ccsc4c3-c3ccc(F)cc3OC(C)C)cc2C1] | LCMS m/z [M + 1]: 543.3 ¹H NMR (400 MHz, CD₃OD) δ 8.68 (s, 1H), 8.35 (s, 1H), 8.17 (d, J = 5.8 Hz, 1H), 8.09 (d, J = 5.6 Hz, 1H), 7.41 (t, J = 7.5 Hz, 1H), 7.03 (dd, J = 11.4, 2.3 Hz, 1H), 6.73-6.93 (m, 2H), 6.28 (d, J = 16.9 Hz, 1H), 5.77-5.84 (m, 2H), 4.81 (m, 2H), 4.54-4.60 (m, 1H), 4.35 (s, 2H), 4.15 (t, J = 5.7 Hz, 2H), 4.11 (s, 3H), 1.08 (d, J = 6.0 Hz, 1H), 0.91 (d, J = 6.0 Hz, 1H). |
| 201 | 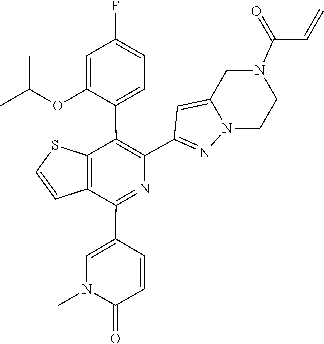 [SMILES: C=CC(=O)N1CCn2nc(-c3nc(-c4ccc(=O)n(C)c4)c4ccsc4c3-c3ccc(F)cc3OC(C)C)cc2C1] | LCMS m/z [M + 1]: 570.3 ¹H NMR (400 MHz, CD₃OD) δ 8.57 (d, J = 2.5 Hz, 1H), 8.13 (dd, J = 9.4, 2.8 Hz, 1H), 8.09 (d, J = 5.6 Hz, 1H), 7.93 (d, J = 5.6 Hz, 1H), 7.39 (t, J = 8.2 Hz, 1H), 6.89 (dt, J = 8.4, 2.3 Hz, 1H), 6.78 (d, J = 9.4 Hz, 2H), 6.28 (d, J = 16.4 Hz, 1H), 5.78-5.90 (m, 2H), 5.77 (s, 1H), 4.80 (m, 2H), 4.53-4.59 (m, 1H), 4.32 (s, 2H), 4.14 (t, J = 5.8 Hz, 2H), 3.74 (s, 3H), 1.08 (d, J = 6.0 Hz, 3H), 0.90 (d, J = 6.0 Hz, 3H). |
| 202 | 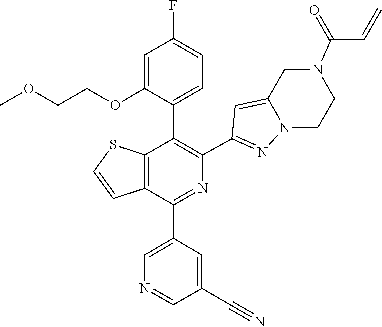 [SMILES: C=CC(=O)N1CCn2nc(-c3nc(-c4cncc(C#N)c4)c4ccsc4c3-c3ccc(F)cc3OCCOC)cc2C1] | LCMS m/z [M + 1]: 581.2 ¹H NMR (400 MHz, CD₃OD) δ 9.41 (d, J = 2.0 Hz, 1H), 9.10 (d, J = 1.8 Hz, 1H), 8.87 (t, J = 1.8 Hz, 1H), 7.92 (d, J = 5.6 Hz, 1H), 7.76 (d, J = 5.6 Hz, 1H), 7.32-7.36 (m, 1H), 7.02 (dd, J = 11.1, 2.2 Hz, 1H), 6.74-6.91 (m, 2H), 6.27 (d, J = 16.6 Hz, 1H), 5.87 (s, 1H), 5.82 (d, J = 9.4 Hz, 1H), 4.75-4.88 (m, 2H), 4.20-4.31 (m, 2H), 4.09-4.20 (m, 2H), 4.02-4.10 (m, 1H), 3.85-3.96 (m, 1H), 3.39 (t, J = 4.6 Hz, 2H), 3.07 (s, 3H). |
| 203 | 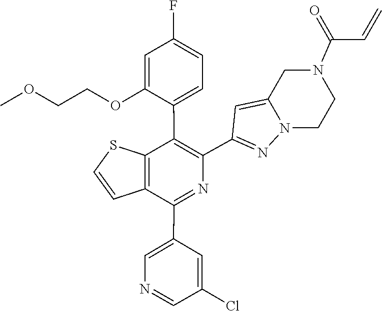 [SMILES: C=CC(=O)N1CCn2nc(-c3nc(-c4cncc(Cl)c4)c4ccsc4c3-c3ccc(F)cc3OCCOC)cc2C1] | LCMS m/z [M + 1]: 590.1 ¹H NMR (400 MHz, CD₃OD) δ 9.08 (d, J = 1.6 Hz, 1H), 8.80 (d, J = 2.4 Hz, 1H), 8.57 (t, J = 2.0 Hz, 1H), 7.94 (d, J = 5.6 Hz, 1H), 7.74 (d, J = 5.6 Hz, 1H), 7.33-7.37 (m, 1H), 7.03 (dd, J = 10.8, 2.0 Hz, 1H), 6.84-6.92 (m, 2H), 6.28 (d, J = 16.4 Hz, 1H), 5.87 (s, 1H), 5.82 (d, J = 10.4 Hz, 1H), 4.77-4.85 (m, 2H), 4.21-4.29 (m, 2H), 4.12-4.14 (m, 2H), 4.04-4.09 (m, 1H), 3.93-3.95 (m, 1H), 3.40 (t, J = 4.4 Hz, 2H), 3.07 (s, 3H). |
| 204 | 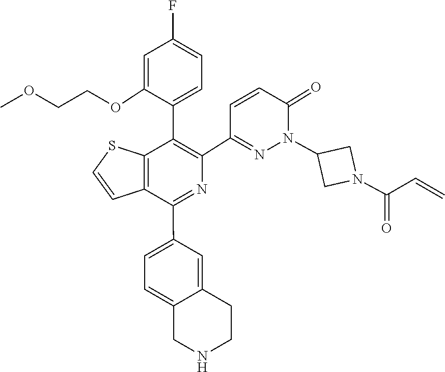 [SMILES: C=CC(=O)N1CC(n2nc(-c3nc(-c4ccc5c(c4)CCNC5)c4ccsc4c3-c3ccc(F)cc3OCCOC)ccc2=O)C1] | LCMS m/z [M + 1]: 638.3 ¹H NMR (400 MHz, CD₃OD) δ 8.23-8.29 (m, 1H), 7.84-7.89 (m, 3H), 7.71-7.73 (m, 1H), 7.37-7.50 (m, 2H), 7.06 (d, J = 9.7 Hz, 1H), 6.81-6.95 (m, 2H), 6.26-6.29 (m, 2H), 5.62-5.83 (m, 2H), 4.50 (s, 2H), 4.43 (t, J = 8.9 Hz, 1H), 4.18-4.23 (m, 1H), 3.95-4.00 (m, 2H), 3.85-3.88 (m, 1H), 3.66-3.79 (m, 1H), 3.58- 3.62 (m, 2H), 3.37-3.42 (m, 2H), 3.27-3.29 (m, 3H), 3.07 (s, 3H). |
| 205 | 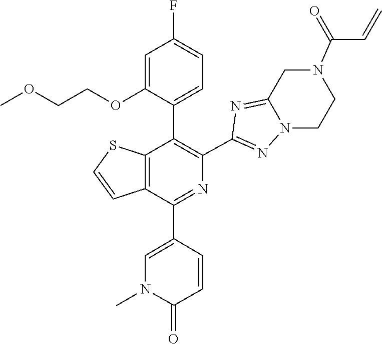 [SMILES: C=CC(=O)N1CCn2nc(-c3nc(-c4ccc(=O)n(C)c4)c4ccsc4c3-c3ccc(F)cc3OCCOC)nc2C1] | LCMS m/z [M + 1]: 587.3 ¹H NMR (400 MHz, CD₃OD) δ 8.51 (s, 1H), 8.25 (dd, J = 9.4, 2.4 Hz, 1H), 7.84 (d, J = 5.6 Hz, 1H), 7.79 (d, J = 5.6 Hz, 1H), 7.32 (t, J = 8.0 Hz, 1H), 6.77- 6.89 (m, 3H), 6.73 (d, J = 9.3 Hz, 1H), 6.29 (d, J = 17.6 Hz, 1H), 5.84 (dd, J = 10.7, 1.7 Hz, 1H), 4.91- 4.94 (m, 2H), 4.12-4.17 (m, 4H), 3.97-4.02 (m, 1H), 3.81-3.86 (m, 1H), 3.73 (s, 3H), 3.37 (t, J = 4.7 Hz, 2H), 3.05 (s, 3H). |
| 206 | 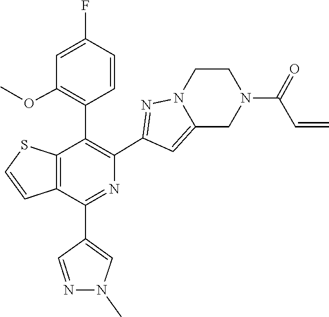 [SMILES: C=CC(=O)N1CCn2nc(-c3nc(-c4cnn(C)c4)c4ccsc4c3-c3ccc(F)cc3OC)cc2C1] | LCMS m/z [M + 1]: 515.2 ¹H NMR (400 MHz, CD₃OD) δ 8.67 (s, 1H), 8.34 (s, 1H), 8.15 (d, J = 5.6 Hz, 1H), 8.08 (d, J = 5.6 Hz, 1H), 7.39 (dd, J = 8.2, 6.7 Hz, 1H), 7.07 (dd, J = 10.9, 2.2 Hz, 1H), 6.72-6.96 (m, 2H), 6.28 (d, J = 16.6 Hz, 1H), 5.77-5.83 (m, 2H), 4.86-4.89 (m, 1H), 4.77 (s, 1H), 4.35 (s, 2H), 4.16 (t, J = 5.7 Hz, 2H), 4.11 (s, 3H), 3.67 (s, 3H). |
| 207 | 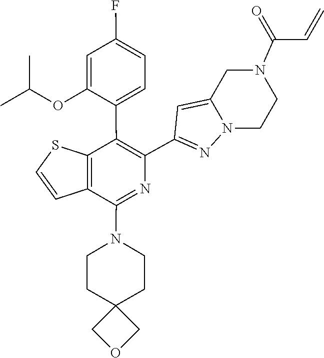 [SMILES: C=CC(=O)N1CCn2nc(-c3nc(N4CCC5(CC4)COC5)c4ccsc4c3-c3ccc(F)cc3OC(C)C)cc2C1] | LCMS m/z [M + 1]: 588.3 ¹H NMR (400 MHz, CD₃OD) δ 7.90 (q, J = 5.7 Hz, 2H), 7.24-7.32 (m, 1H), 7.02 (dd, J = 11.4, 2.1 Hz, 1H), 6.69-6.92 (m, 2H), 6.28 (d, J = 16.5 Hz, 1H), 5.74-5.89 (m, 1H), 5.57 (s, 1H), 4.70-4.84 (m, 2H), 4.49-4.65 (m, 5H), 4.31 (s, 2H), 4.14 (t, J = 5.4 Hz, 2H),3.83-3.96 (m, 4H), 2.16-2.33 (m, 4H), 1.08 (d, J = 6.0 Hz, 3H), 0.95 (d, J = 6.0 Hz, 3H). |
| 208 | 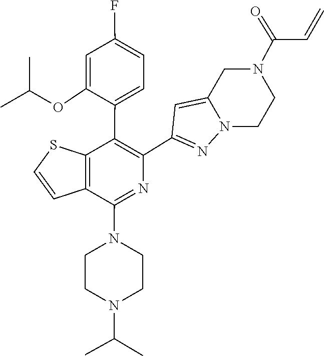 [SMILES: C=CC(=O)N1CCn2nc(-c3nc(N4CCN(C(C)C)CC4)c4ccsc4c3-c3ccc(F)cc3OC(C)C)cc2C1] | LCMS m/z [M + 1]: 589.3 ¹H NMR (400 MHz, CD₃OD) δ 7.66 (dd, J = 14.0, 5.2 Hz, 2H), 7.20-7.24 (m, 1H), 6.75-6.88 (m, 3H), 6.27 (d, J = 16.4 Hz, 1H), 5.73-5.81 (m, 2H), 4.77 (m, 2H), 4.44-4.50 (m, 3H), 4.20 (s, 2H), 4.12 (d, J = 5.2 Hz, 2H), 3.41-3.70 (m, 7H), 1.45 (d, J = 6.4 Hz, 6H), 1.05 (d, J = 6.0 Hz, 3H), 0.89 (d, J = 6.0 Hz, 3H). |
| 209 | 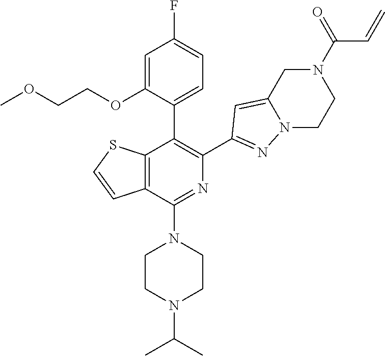 [SMILES: C=CC(=O)N1CCn2nc(-c3nc(N4CCN(C(C)C)CC4)c4ccsc4c3-c3ccc(F)cc3OCCOC)cc2C1] | LCMS m/z [M + 1]: 605.3 ¹H NMR (400 MHz, CD₃OD) δ7.68 (dd, J = 15.2, 5.6 Hz, 2H), 7.19-7.23 (m, 1H), 6.96 (d, J = 11.2 Hz, 1H), 6.74-6.91 (m, 2H), 6.27 (d, J = 17.2 Hz, 1H), 5.82 (d, J = 12.8 Hz, 2H), 4.79 (m, 2H), 4.26-4.64 (m, 2H), 4.20 (s, 2H), 4.1-4.13 (m, 2H), 3.99-4.04 (m, 1H), 3.89-3.94 (m, 1H), 3.43-3.75 (m, 7H), 3.38 (t, J = 4.4 Hz, 2H), 3.05 (s, 3H), 1.45 (d, J = 6.8 Hz, 6H). |
| 210 | 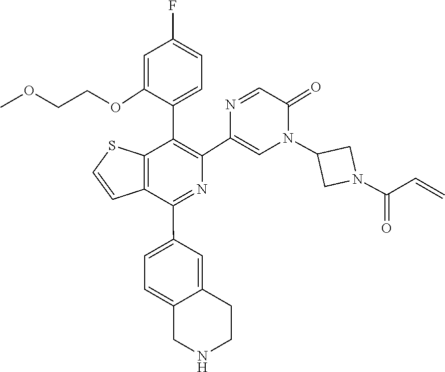 [SMILES: C=CC(=O)N1CC(n2cc(-c3nc(-c4ccc5c(c4)CCNC5)c4ccsc4c3-c3ccc(F)cc3OCCOC)ncc2=O)C1] | LCMS m/z [M + 1]: 638.3 ¹H NMR (400 MHz, CD₃OD) δ 8.37 (d, J = 4.4 Hz, 1H), 8.23 (d, J = 2.4 Hz, 1H), 7.93 (d, J = 5.6 Hz, 1H), 7.88 (d, J = 5.8 Hz, 2H), 7.74 (d, J = 5.6 Hz, 1H), 7.49 (d, J = 8.5 Hz, 1H), 7.38 (t, J = 7.9 Hz, 1H), 6.92 (d, J = 11.0 Hz, 1H), 6.84 (dt, J = 8.2, 2.0 Hz, 1H), 6.23- 6.38 (m, 2H), 5.75 (dd, J = 10.0, 1.9 Hz, 1H), 5.43- 5.48 (m, 1H), 4.71 (t, J = 9.7 Hz, 1H), 4.44-4.51 (m, 3H), 4.32 (dd, J = 10.5, 3.5 Hz, 1H), 3.98-4.07 (m, 2H), 3.85-3.89 (m, 1H), 3.60 (t, J = 6.3 Hz, 2H), 3.41 (t, J = 4.5 Hz, 2H), 3.28 (d, J = 6.1 Hz, 2H), 3.11 (s, 3H). |
| 211 | 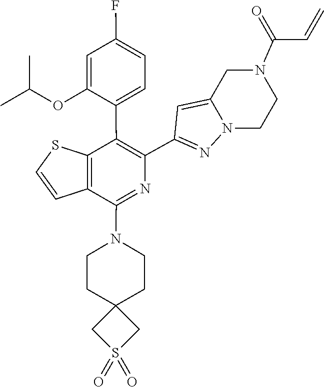 [SMILES: C=CC(=O)N1CCn2nc(-c3nc(N4CCC5(CC4)CS(=O)(=O)C5)c4ccsc4c3-c3ccc(F)cc3OC(C)C)cc2C1] | LCMS m/z [M + 1]: 636.3 ¹H NMR (400 MHz, CD₃OD) δ 7.89-7.93 (m, 2H), 7.28 (t, J = 7.2 Hz, 1H), 7.02 (dd, J = 11.4, 1.8 Hz, 1H), 6.72-6.88 (m, 2H), 6.27 (d, J = 16.4 Hz, 1H), 5.81 (s, 1H), 5.58 (s, 1H), 4.78 (m, 2H), 4.54-4.60 (m, 1H), 4.30 (s, 2H), 4.12-4.15 (m, 6H), 3.95-3.97 (m, 4H), 2.25-2.28 (m, 4H), 1.08 (d, J = 6.0 Hz, 3H), 0.94 (d, J = 6.0 Hz, 3H). |
| 212 | 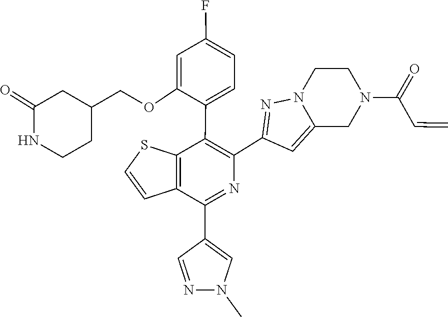 [SMILES: C=CC(=O)N1CCn2nc(-c3nc(-c4cnn(C)c4)c4ccsc4c3-c3ccc(F)cc3OCC3CCNC(=O)C3)cc2C1] | LCMS m/z [M + 1]: 612.2 ¹H NMR (400 MHz, CD₃OD) δ 8.68 (d, J = 2.5 Hz, 1H), 8.36 (d, J = 2.5 Hz, 1H), 8.07-8.14 (m, 1H), 7.43-7.48 (m, 1H), 7.08-7.11 (m, 1H), 6.96-7.00 (m, 1H), 6.77-6.95 (m, 1H), 6.28 (d, J = 16.7 Hz, 1H), 5.77-5.85 (m, 2H), 4.77-4.90 (m, 2H), 4.28-4.51 (m, 2H), 4.11-4.18 (m, 5H), 3.80-3.94 (m, 2H), 3.00-3.12 (m, 2H), 1.93-2.03 (m, 2H), 1.58-1.75 (m, 2H), 1.13- 1.28 (m, 1H). |
| 213 | 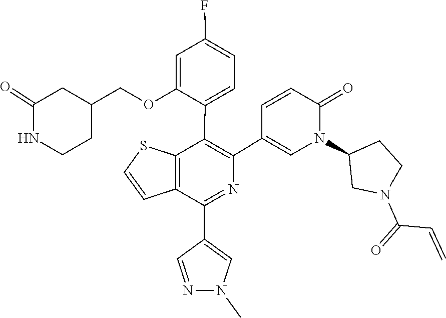 [SMILES: C=CC(=O)N1CC[C@H](n2cc(-c3nc(-c4cnn(C)c4)c4ccsc4c3-c3ccc(F)cc3OCC3CCNC(=O)C3)ccc2=O)C1] | LCMS m/z [M + 1]: 653.3 ¹H NMR (400 MHz, CD₃OD) δ 8.60 (s, 1H), 8.28- 8.35 (m, 2H), 8.00-8.15 (m, 2H), 7.75-7.85 (m, 1H), 7.25-7.45 (m, 1H), 6.95-7.05 (m, 1H), 6.80-6.90 (m, 2H), 6.50-6.70 (m, 1H), 6.25-6.30 (m, 1H), 5.75 (t, J = 10.0 Hz, 1H), 5.55-5.65 (m, 1H), 4.09 (s, 3H), 3.70- 4.00 (m, 6H), 3.05-3.20 (m, 2H), 2.00-2.40 (m, 4H), 1.60-1.90 (m, 2H), 1.20-1.35 (m, 1H). |
| 213a | 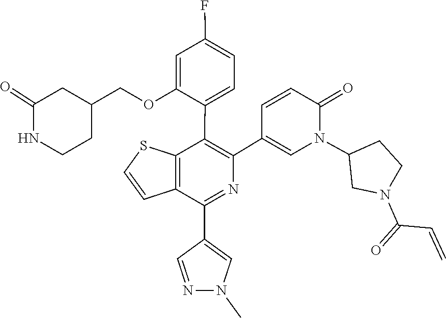 [SMILES: C=CC(=O)N1CCC(n2cc(-c3nc(-c4cnn(C)c4)c4ccsc4c3-c3ccc(F)cc3OCC3CCNC(=O)C3)ccc2=O)C1] | |
| 214 | 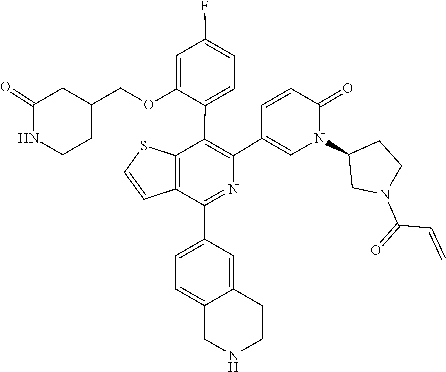 [SMILES: C=CC(=O)N1CC[C@H](n2cc(-c3nc(-c4ccc5c(c4)CCNC5)c4ccsc4c3-c3ccc(F)cc3OCC3CCNC(=O)C3)ccc2=O)C1] | LCMS m/z [M + 1]: 704.4 ¹H NMR (400 MHz, CD₃OD) δ 8.25-8.35 (m, 1H), 7.80-8.00 (m, 4H), 7.71-7.74 (m, 1H), 7.50 (d, J = 8.4 Hz, 1H), 7.25-7.40 (m, 1H), 6.95-7.05 (m, 1H), 6.80- 6.90 (m, 1H), 6.72-6.80 (m, 1H), 6.50-6.70 (m, 1H), 6.27 (d, J = 17.6 Hz, 1H), 5.76 (t, J = 10.0 Hz, 1H), 5.59 (d, J = 17.1 Hz, 1H), 4.51 (s, 2H), 3.55-4.00 (m, 8H), 3.25-3.30 (m, 2H), 3.05-3.20 (m, 2H), 2.00-2.40 (m, 4H), 1.60-1.90 (m, 2H), 1.20-1.35 (m, 1H). |
| 214a | 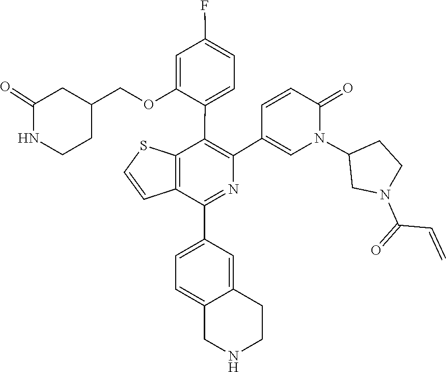 [SMILES: C=CC(=O)N1CCC(n2cc(-c3nc(-c4ccc5c(c4)CCNC5)c4ccsc4c3-c3ccc(F)cc3OCC3CCNC(=O)C3)ccc2=O)C1] | |
| 215 | 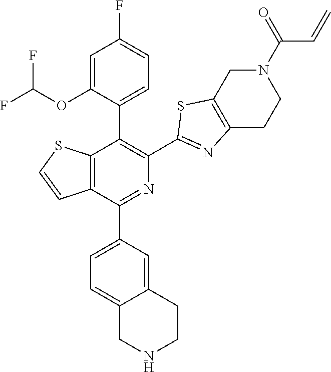 [SMILES: C=CC(=O)N1CCc2nc(-c3nc(-c4ccc5c(c4)CCNC5)c4ccsc4c3-c3ccc(F)cc3OC(F)F)sc2C1] | LCMS m/z [M + 1]: 619.3 ¹H NMR (400 MHz, CD₃OD) δ 7.89-7.92 (m, 2H), 7.83 (d, J = 5.6 Hz, 1H), 7.74 (d, J = 5.6 Hz, 1H), 7.40-7.50 (m, 2H), 7.10-7.20 (m, 2H), 6.75-6.90 (m, 1H), 6.44-6.63 (m, 1H), 6.20-6.30 (m, 1H), 5.75-5.85 (m, 1H), 4.80-4.85 (m, 2H), 4.51 (s, 2H), 3.80-3.95 (m, 2H), 3.61 (t, J = 6.4 Hz, 1H), 3.25-3.30 (m, 2H), 2.50-2.75 (m, 2H). |
| 216 | 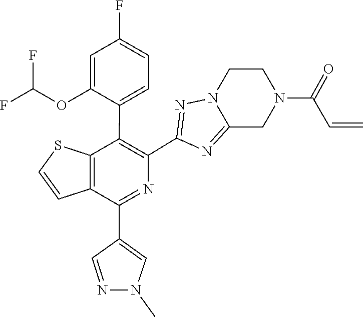 [SMILES: C=CC(=O)N1CCn2nc(-c3nc(-c4cnn(C)c4)c4ccsc4c3-c3ccc(F)cc3OC(F)F)nc2C1] | LCMS m/z [M + 1]: 552.3 ¹H NMR (400 MHz, CD₃OD) δ 8.58 (s, 1H), 8.35 (s, 1H), 7.99-8.06 (m, 2H), 7.43-7.52 (m, 1H), 7.08-7.19 (m, 2H), 6.53-6.96 (m, 2H), 6.3 (d, J = 16.6 Hz, 1H), 5.81-5.88 (m, 1H), 4.92-5.04 (m, 2H), 4.1-4.23 (m, 4H), 4.07 (s, 3H). |
| 217 | 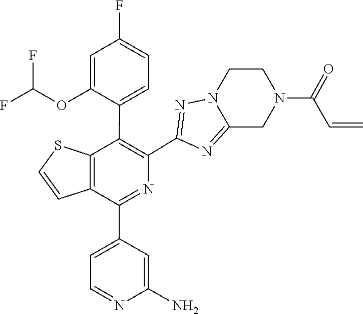 [SMILES: C=CC(=O)N1CCn2nc(-c3nc(-c4ccnc(N)c4)c4ccsc4c3-c3ccc(F)cc3OC(F)F)nc2C1] | LCMS m/z [M + 1]: 564.2 ¹H NMR (400 MHz, CD₃OD) δ 8.02 (t, J = 6.7 Hz, 2H), 7.85 (d, J = 5.6 Hz, 1H), 7.64 (s, 1H), 7.59 (d, J = 7.0 Hz, 1H), 7.46 (t, J = 6.8 Hz, 1H), 7.09-7.18 (m, 2H), 6.54-6.95 (m, 2H), 6.3 (d, J = 16.7 Hz, 1H), 5.85 (d, J = 10.6 Hz, 1H), 4.91-4.97 (m, 2H), 4.15 (s, 4H). |
| 218 | 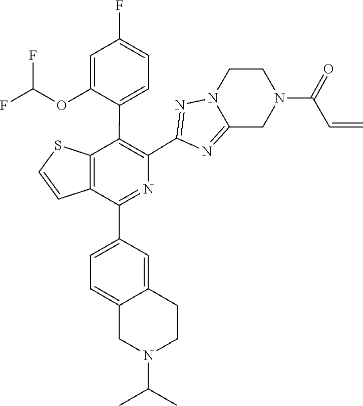 [SMILES: C=CC(=O)N1CCn2nc(-c3nc(-c4ccc5c(c4)CCN(C(C)C)C5)c4ccsc4c3-c3ccc(F)cc3OC(F)F)nc2C1] | LCMS m/z [M + 1]: 645.3 ¹H NMR (400 MHz, CD₃OD) δ 7.88-8.02 (m, 3H), 7.75 (d, J = 5.6 Hz, 1H), 7.42-7.55 (m, 2H), 7.07-7.17 (m, 2H), 6.53-6.96 (m, 2H), 6.29 (d, J = 16.7 Hz, 1H), 5.84 (d, J = 10.6 Hz, 1H), 4.95-5.03 (m, 1H), 4.6 (s, 2H), 4.15 (s, 4H), 3.72-3.9 (m, 2H), 3.32-3.58 (m, 3H), 1.51 (d, J = 6.6 Hz, 6H). |
| 219 | 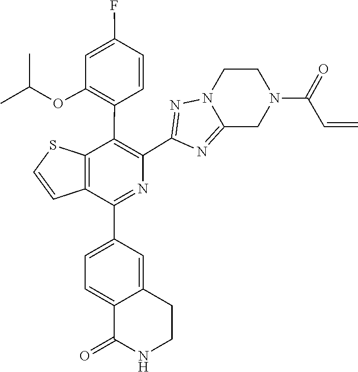 [SMILES: C=CC(=O)N1CCn2nc(-c3nc(-c4ccc5c(c4)CCNC5=O)c4ccsc4c3-c3ccc(F)cc3OC(C)C)nc2C1] | LCMS m/z [M + 1]: 609.3 ¹H NMR (400 MHz, CD₃OD) δ 8.18 (d, J = 8.1 Hz, 1H), 7.98-8.00 (m, 3H), 7.80 (d, J = 5.6 Hz, 1H), 7.40 (t, J = 7.8 Hz, 1H), 6.79-6.87 (m, 3H), 6.30 (d, J = 16.2 Hz, 1H), 5.85 (d, J = 10.6 Hz, 1H), 4.95-4.97 (m, 2H), 4.45-4.51 (m, 1H), 4.16 (s, 4H), 3.61 (t, J = 6.6 Hz, 2H), 3.16 (t, J = 6.5 Hz, 2H), 1.06 (d, J = 6.0 Hz, 3H), 0.90 (d, J = 5.9 Hz, 3H). |
| 220 | 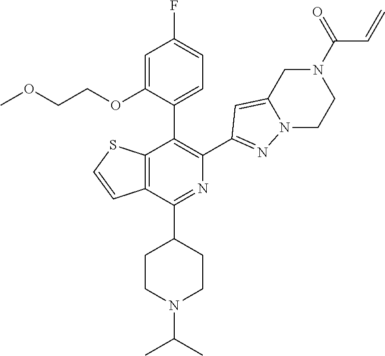 [SMILES: C=CC(=O)N1CCn2nc(-c3nc(C4CCN(C(C)C)CC4)c4ccsc4c3-c3ccc(F)cc3OCCOC)cc2C1] | LCMS m/z [M + 1]: 604.3 ¹H NMR (400 MHz, CD₃OD) δ 7.67-7.78 (m, 2H), 7.22 (t, J = 8.0 Hz, 1H), 6.95 (d, J = 10.8 Hz, 1H), 6.73-6.86 (m, 2H), 6.27 (d, J = 16.8 Hz, 1H), 5.81- 5.91 (m, 2H), 4.69-4.76 (m, 2H), 4.11-4.19 (m, 4H), 3.98-4.03 (m, 1H), 3.88-3.93 (m, 1H), 3.72-3.80 (m, 1H), 3.57-3.64 (m, 3H), 3.36-3.38 (m, 3H), 3.02 (s, 3H), 2.43-2.52 (m, 2H), 2.25-2.28 (m, 2H), 1.44 (d, J = 6.8 Hz, 6H). |
| 221 | 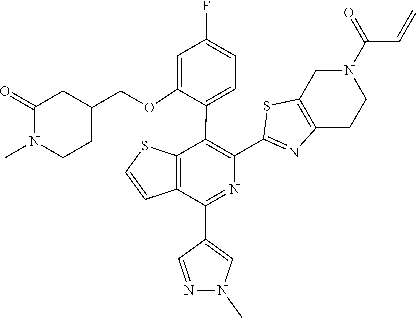 [SMILES: C=CC(=O)N1CCc2nc(-c3nc(-c4cnn(C)c4)c4ccsc4c3-c3ccc(F)cc3OCC3CCN(C)C(=O)C3)sc2C1] | LCMS m/z [M + 1]: 643.3 ¹H NMR (400 MHz, CD₃OD) δ 8.49 (d, J = 3.9 Hz, 1H), 8.29 (d, J = 2.6 Hz, 1H), 7.95 (d, J = 5.6 Hz, 1H), 7.88 (t, J = 4.3 Hz, 1H), 7.35 (t, J = 7.6 Hz, 1H), 6.83-6.98 (m, 3H), 6.25 (t, J = 17.1 Hz, 1H), 5.80 (d, J = 10.2 Hz, 1H), 4.84-4.86 (m, 2H), 4.06 (s, 3H), 3.69-3.93 (m, 4H), 3.02-3.08 (m, 2H), 2.75-2.80 (m, 5H), 1.92-2.06 (m, 3H), 1.49-1.77 (m, 2H). |
| 222 | 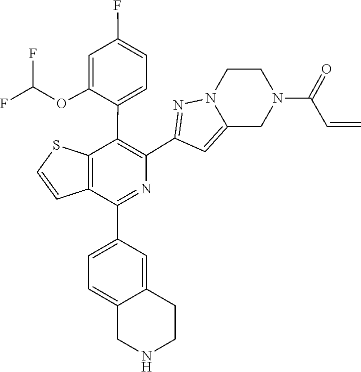 [SMILES: C=CC(=O)N1CCn2nc(-c3nc(-c4ccc5c(c4)CCNC5)c4ccsc4c3-c3ccc(F)cc3OC(F)F)cc2C1] | LCMS m/z [M + 1]: 602.2 ¹H NMR (400 MHz, CD₃OD) δ 7.91 (d, J = 5.4 Hz, 3H), 7.73 (d, J = 5.6 Hz, 1H), 7.49-7.52 (m, 2H), 7.17-7.20 (m, 2H), 6.72-6.91 (m, 2H), 6.28 (d, J = 16.6 Hz, 1H), 6.03-6.06 (m, 1H), 5.82 (d, J = 10.4 Hz, 1H), 4.79 (s, 1H), 4.52 (s, 2H), 4.11-4.18 (m, 4H), 3.61 (t, J = 6.2 Hz, 2H), 3.28-3.30 (m, 2H). |
| 223 | 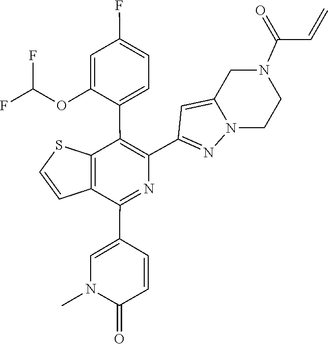 [SMILES: C=CC(=O)N1CCn2nc(-c3nc(-c4ccc(=O)n(C)c4)c4ccsc4c3-c3ccc(F)cc3OC(F)F)cc2C1] | LCMS m/z [M + 1]: 578.3 ¹H NMR (400 MHz, CD₃OD) δ 8.52 (d, J = 2.0 Hz, 1H), 8.18 (dd, J = 9.4, 2.3 Hz, 1H), 8.0 (d, J = 5.6 Hz, 1H), 7.89 (d, J = 5.6 Hz, 1H), 7.19-7.23 (m, 1H), 6.56-6.93 (m, 3H), 6.28 (d, J = 16.4 Hz, 1H), 5.97 (s, 1H), 5.82 (d, J = 9.5 Hz, 1H), 4.78 (s, 1H), 4.07-4.23 (m, 4H), 3.74 (s, 3H). |
| 224 | 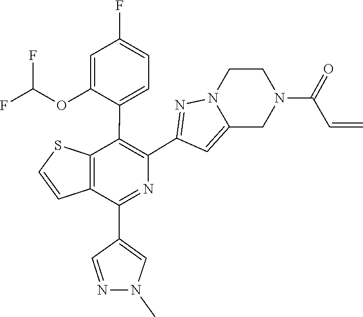 [SMILES: C=CC(=O)N1CCn2nc(-c3nc(-c4cnn(C)c4)c4ccsc4c3-c3ccc(F)cc3OC(F)F)cc2C1] | LCMS m/z [M + 1]: 551.2 ¹H NMR (400 MHz, CD₃OD) δ 8.64 (s, 1H), 8.34 (s, 1H), 8.09 (dd, J = 14.1, 1.6 Hz, 2H), 7.57 (t, J = 8.0 Hz, 1H), 7.21-7.26 (m, 2H), 6.60-6.96 (m, 2H), 6.28 (d, J = 16.7 Hz, 1H), 5.82-5.90 (m, 2H), 4.78-4.85 (m, 2H), 4.25-4.28 (m, 2H), 4.12-4.15 (m, 2H), 4.10 (s, 3H). |
| 225 | 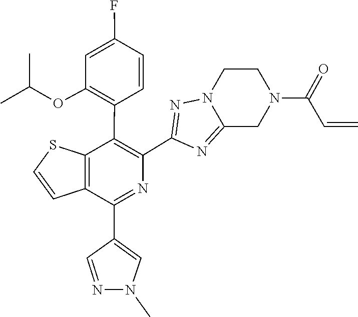 [SMILES: C=CC(=O)N1CCn2nc(-c3nc(-c4cnn(C)c4)c4ccsc4c3-c3ccc(F)cc3OC(C)C)nc2C1] | LCMS m/z [M + 1]: 544.2 ¹H NMR (400 MHz, CD₃OD) δ 8.62 (s, 1H), 8.35 (s, 1H), 8.08 (dd, J = 16.7, 5.7 Hz, 2H), 7.39 (t, J = 8.1 Hz, 1H), 6.79-6.89 (m, 3H), 6.31 (d, J = 16.7 Hz, 1H), 5.85-5.88 (m, 1H), 4.98-5.01 (m, 2H), 4.46-4.52 (m, 1H), 4.09-4.17 (m, 7H), 1.05 (d, J = 6.0 Hz, 3H), 0.89 (d, J = 5.9 Hz, 3H). |
| 226 | 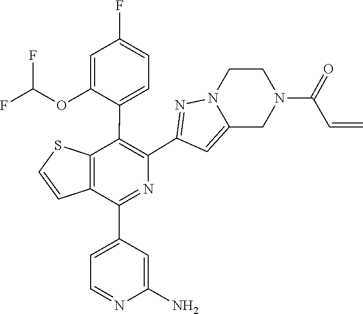 [SMILES: C=CC(=O)N1CCn2nc(-c3nc(-c4ccnc(N)c4)c4ccsc4c3-c3ccc(F)cc3OC(F)F)cc2C1] | LCMS m/z [M + 1]: 563.3 ¹H NMR (400 MHz, CD₃OD) δ 8.02 (d, J = 6.7 Hz, 1H), 7.92 (d, J = 5.6 Hz, 1H), 7.80 (d, J = 5.6 Hz, 1H), 7.63 (s, 1H), 7.56 (d, J = 6.3 Hz, 1H), 7.46 (t, J = 6.9 Hz, 1H), 7.14-7.18 (m, 2H), 6.50-6.86 (m, 2H), 6.18-6.30 (m, 2H), 5.82 (d, J = 10.7 Hz, 1H), 4.80-4.88 (m, 2H), 4.08-4.13 (m, 4H). |
| 227 | 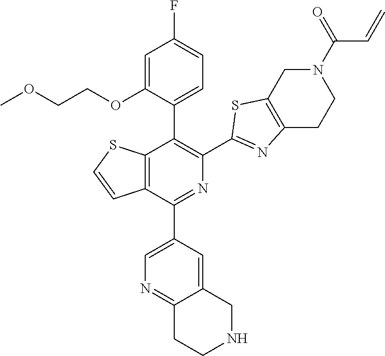 [SMILES: C=CC(=O)N1CCc2nc(-c3nc(-c4cnc5c(c4)CNCC5)c4ccsc4c3-c3ccc(F)cc3OCCOC)sc2C1] | LCMS m/z [M + 1]: 628.2 ¹H NMR (400 MHz, CD₃OD) δ 9.12 (d, J = 2.0 Hz, 1H), 8.36 (d, J = 1.9 Hz, 1H), 7.90 (d, J = 5.6 Hz, 1H), 7.77 (d, J = 5.6 Hz, 1H), 7.37 (dd, J = 8.4, 6.6 Hz, 1H), 6.95 (d, J = 11.3 Hz, 1H), 6.82-6.87 (m, 2H), 6.25 (d, J = 16.8 Hz, 1H), 5.80 (d, J = 9.7 Hz, 1H), 4.62 (s, 1H), 4.01-4.05 (m, 1H), 3.82-3.96 (m, 3H), 3.74 (t, J = 6.5 Hz, 1H), 3.35-3.41 (m, 2H), 3.27- 3.33 (m, 4H), 3.00 (s, 3H), 2.67-2.76 (m, 2H). |
| 228 | 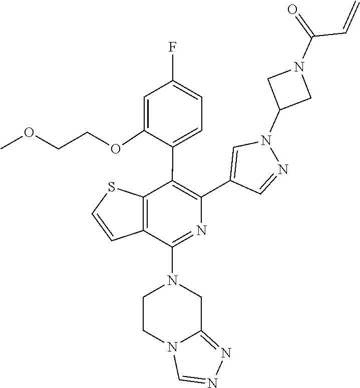 [SMILES: C=CC(=O)N1CC(n2cc(-c3nc(N4CCn5cnnc5C4)c4ccsc4c3-c3ccc(F)cc3OCCOC)cn2)C1] | LCMS m/z [M + 1]: 601.2 ¹H NMR (400 MHz, CD₃OD) δ 8.95 (s, 1H), 7.62 (q, J = 5.6 Hz, 2H), 7.43 (d, J = 3.4 Hz, 1H), 7.34 (d, J = 3.4 Hz, 1H), 7.22 (dd, J = 8.3, 6.8 Hz, 1H), 7.03 (dd, J = 11.1, 2.3 Hz, 1H), 6.88 (td, J = 8.3, 2.4 Hz, 1H), 6.23-6.41 (m, 2H), 5.77 (dd, J = 9.9, 2.3 Hz, 1H), 5.14-5.20 (m, 1H), 5.08 (s, 2H), 4.71 (t, J = 8.7 Hz, 1H), 4.43-4.57 (m, 2H), 4.37 (t, J = 5.4 Hz, 2H), 4.26 (dd, J = 10.8, 5.1 Hz, 1H), 4.19 (t, J = 5.4 Hz, 2H), 3.93- 4.04 (m, 2H), 3.36 (t, J = 4.6 Hz, 2H), 2.98 (s, 3H). |
| 229 | 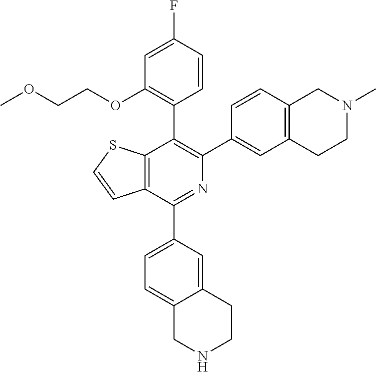 | LCMS m/z [M + 1]: 580.2 ¹H NMR (500 MHz, CD₃OD) δ 7.88-7.77 (m, 3H), 7.67 (d, J = 5.6 Hz, 1H), 7.47 (d, J = 7.9 Hz, 1H), 7.44 (s, 1H), 7.39 (dd, J = 8.0, 1.8 Hz, 1H), 7.25-7.19 (m, 1H), 7.11 (d, J = 8.0 Hz, 1H), 6.93-6.87 (m, 1H), 6.74 (d, J = 7.3 Hz, 1H), 4.50 (s, 2H), 4.04 (d, J = 8.1 Hz, 1H), 3.90 (s, 2H), 3.73 (s, 1H), 3.60 (t, J = 6.3 Hz, 2H), 3.48 (dd, J = 10.9, 5.7 Hz, 3H), 3.27 (d, J = 6.6 Hz, 2H), 3.15 (s, 3H), 3.03 (s, 2H), 2.83 (s, 1H), 2.66 (s, 1H), 2.03 (s, 1H), 1.29 (s, 1H). |
| 230 | 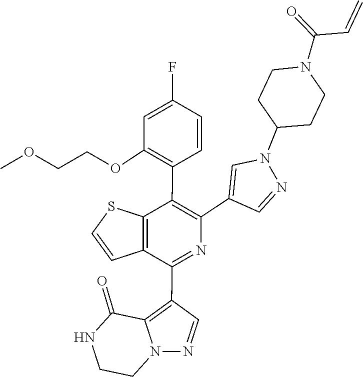 [SMILES: C=CC(=O)N1CCC(n2cc(-c3nc(-c4cnn5c4C(=O)NCC5)c4ccsc4c3-c3ccc(F)cc3OCCOC)cn2)CC1] | LCMS m/z [M + 1]: 642.3 ¹H NMR (400 MHz, CD₃OD) δ 8.77 (s, 1H), 8.29 (d, J = 5.8 Hz, 1H), 8.18 (d, J = 5.7 Hz, 1H), 7.97 (s, 1H), 7.57 (s, 1H), 7.42-7.45 (m, 1H), 7.12 (dd, J = 2.3, 11.0 Hz, 1H), 6.95-7.00 (m, 1H), 6.80 (dd, J = 10.6, 16.8 Hz, 1H), 6.22 (dd, J = 1.9, 16.8 Hz, 1H), 5.77 (dd, J = 1.9, 10.6 Hz, 1H), 4.62-4.65 (m, 3H), 4.46-4.54 (m, 1H), 4.20-4.23 (m, 1H), 4.05-4.10 (m, 1H), 3.90- 3.98 (m, 3H), 3.40 (t, J = 4.6 Hz, 2H), 3.08 (s, 3H), 2.90-2.98 (m, 1H), 2.14-2.17 (m, 2H), 1.85-1.91 (m, 2H). |
| 231 | 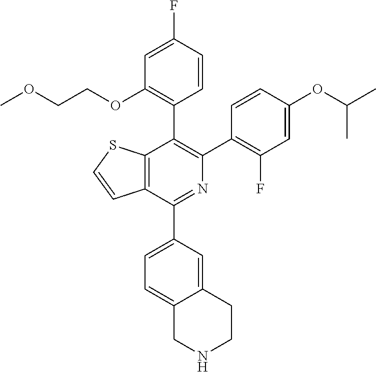 [SMILES: COCCOc1cc(F)ccc1-c1c(-c2ccc(OC(C)C)cc2F)nc(-c2ccc3c(c2)CCNC3)c2ccsc12] | LCMS m/z [M + 1]: 587.7 ¹H NMR (500 MHz, CD₃OD) δ 8.27 (dt, J = 5.6, 1.7 Hz, 1H), 7.93-7.80 (m, 3H), 7.63 (d, J = 8.4 Hz, 1H), 7.56-7.44 (m, 1H), 7.33-7.16 (m, 1H), 7.10-6.82 (m, 2H), 6.82-6.66 (m, 1H), 4.67 (dp, J = 46.7, 6.1 Hz, 1H), 4.58 (s, 2H), 4.16 (dddd, J = 26.0, 10.5, 6.6, 4.2 Hz, 1H), 4.00 (ddd, J = 11.0, 5.1, 3.3 Hz, 1H), 3.64 (t, J = 6.3 Hz, 2H), 3.60-3.50 (m, 2H), 3.17 (d, J = 16.1 Hz, 3H), 1.30 (d, J = 6.0 Hz, 2H), 1.24 (t, J = 6.4 Hz, 3H), 1.08 (d, J = 5.9 Hz, 1H). |
| 232 | 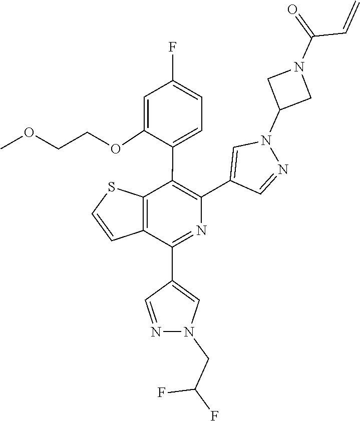 [SMILES: C=CC(=O)N1CC(n2cc(-c3nc(-c4cnn(CC(F)F)c4)c4ccsc4c3-c3ccc(F)cc3OCCOC)cn2)C1] | LCMS m/z [M + 1]: 609.1 ¹H NMR (500 MHz, CD₃OD) 8.50 (s, 1H), 8.32 (d, J = 0.7 Hz, 1H), 7.82 (d, J = 5.6 Hz, 1H), 7.76 (d, J = 5.9 Hz, 1H), 7.67 (d, J = 5.6 Hz, 1H), 7.43 (d, J = 6.9 Hz, 1H), 7.31 (dd, J = 8.4, 6.7 Hz, 1H), 7.04 (dd, J = 11.1, 2.5 Hz, 1H), 6.91 (td, J = 8.3, 2.5 Hz, 1H), 6.44- 6.24 (m, 3H), 5.77 (dd, J = 10.1, 2.1 Hz, 1H), 5.24 (qd, J = 6.8, 6.2, 3.4 Hz, 1H), 4.75-4.67 (m, 2H), 4.57 (dd, J = 9.7, 5.2 Hz, 1H), 4.49 (ddd, J = 9.5, 8.2, 1.4 Hz, 1H), 4.32 (dd, J = 11.1, 5.3 Hz, 1H), 4.03 (dt, J = 10.9, 4.5 Hz, 1H), 3.97 (dtd, J = 11.1, 4.7, 1.3 Hz, 1H), 3.45-3.34 (m, 3H), 3.01 (s, 2H), 2.87-2.80 (m, 1H). |
| 233 | 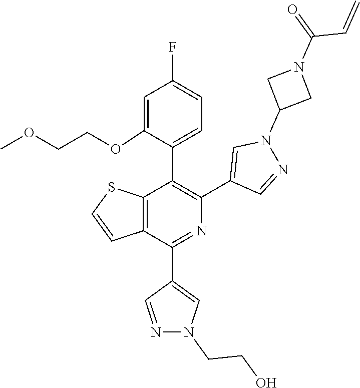 [SMILES: C=CC(=O)N1CC(n2cc(-c3nc(-c4cnn(CCO)c4)c4ccsc4c3-c3ccc(F)cc3OCCOC)cn2)C1] | LCMS m/z [M + 1]: 589.1 ¹H NMR (500 MHz, CD₃OD) δ 8.64 (s, 1H), 8.35 (d, J = 0.8 Hz, 1H), 8.07-8.01 (m, 2H), 7.99 (d, J = 3.1 Hz, 1H), 7.63 (d, J = 5.0 Hz, 1H), 7.37 (dd, J = 8.4, 6.5 Hz, 1H), 7.07 (dd, J = 11.1, 2.4 Hz, 1H), 6.91 (td, J = 8.3, 2.4 Hz, 1H), 6.37 (dd, J = 17.0, 10.1 Hz, 1H), 6.29 (dd, J = 17.0, 2.2 Hz, 1H), 5.78 (dd, J = 10.1, 2.2 Hz, 1H), 5.29 (ddd, J = 13.2, 8.2, 5.1 Hz, 1H), 4.76 (ddd, J = 9.5, 8.0, 1.4 Hz, 1H), 4.62-4.56 (m, 1H), 4.51 (ddd, J = 10.9, 8.1, 1.4 Hz, 1H), 4.43 (t, J = 5.2 Hz, 2H), 4.31 (dd, J = 11.0, 5.1 Hz, 1H), 4.08 (dt, J = 11.0, 4.1 Hz, 1H), 4.04-3.96 (m, 3H), 3.44 (dd, J = 5.2, 3.8 Hz, 2H), 3.11 (s, 3H). |
| 234 | 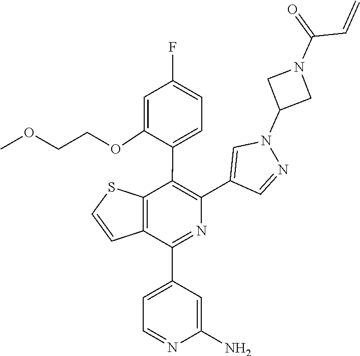 [SMILES: C=CC(=O)N1CC(n2cc(-c3nc(-c4ccnc(N)c4)c4ccsc4c3-c3ccc(F)cc3OCCOC)cn2)C1] | LCMS m/z [M + 1]: 571.6 ¹H NMR (500 MHz, CD₃OD) δ 8.09 (t, J = 5.0 Hz, 1H), 7.78-7.66 (m, 3H), 7.51 (d, J = 6.6 Hz, 1H), 7.39-7.22 (m, 4H), 7.09 (dt, J = 11.1, 2.0 Hz, 1H), 6.95 (td, J = 8.3, 2.4 Hz, 1H), 6.43-6.24 (m, 2H), 5.79 (dd, J = 10.2, 2.2 Hz, 1H), 5.29-5.18 (m, 1H), 4.75 (ddd, J = 9.5, 8.0, 1.4 Hz, 1H), 4.58 (dd, J = 9.7, 5.1 Hz, 1H), 4.50 (ddd, J = 10.9, 8.1, 1.3 Hz, 1H), 4.33 (dd, J = 11.0, 5.2 Hz, 1H), 4.12-3.97 (m, 2H), 3.41 (t, J = 4.6 Hz, 2H), 3.04 (s, 3H). |
| 235 | 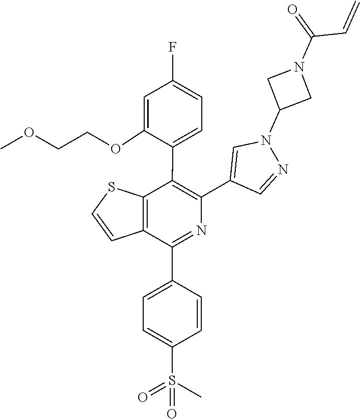 [SMILES: C=CC(=O)N1CC(n2cc(-c3nc(-c4ccc(S(C)(=O)=O)cc4)c4ccsc4c3-c3ccc(F)cc3OCCOC)cn2)C1] | LCMS m/z [M + 1]: 533.7 ¹H NMR (500 MHz, CD₃OD) δ 8.22 (q, J = 8.5 Hz, 4H), 7.79 (dd, J = 8.9, 5.5 Hz, 2H), 7.69 (d, J = 5.6 Hz, 1H), 7.50 (d, J = 6.6 Hz, 1H), 7.36 (dd, J = 8.4, 6.6 Hz, 1H), 7.10 (dd, J = 11.1, 2.4 Hz, 1H), 6.95 (td, J = 8.3, 2.4 Hz, 1H), 6.43-6.23 (m, 2H), 5.78 (dd, J = 10.2, 2.1 Hz, 1H), 5.33-5.19 (m, 1H), 4.75 (ddd, J = 9.5, 8.0, 1.4 Hz, 1H), 4.58 (dd, J = 9.7, 5.1 Hz, 1H), 4.54-4.46 (m, 1H), 4.33 (dd, J = 11.0, 5.3 Hz, 1H), 4.14-3.93 (m, 2H), 3.43 (t, J = 4.6 Hz, 2H), 3.25 (s, 3H), 3.06 (s, 3H). |
| 236 | 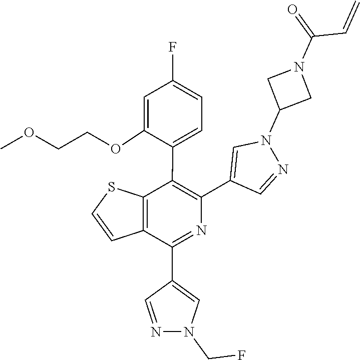 [SMILES: C=CC(=O)N1CC(n2cc(-c3nc(-c4cnn(CF)c4)c4ccsc4c3-c3ccc(F)cc3OCCOC)cn2)C1] | LCMS m/z [M + 1]: 577.1 ¹H NMR (500 MHz, CD₃OD) δ 8.77 (d, J = 5.5 Hz, 1H), 8.48-8.44 (m, 1H), 7.85 (t, J = 6.1 Hz, 1H), 7.81 (dd, J = 6.6, 2.8 Hz, 1H), 7.69 (t, J = 5.9 Hz, 1H), 7.41 (dd, J = 7.9, 4.0 Hz, 1H), 7.31 (ddd, J = 8.3, 6.6, 3.3 Hz, 1H), 7.07 (dt, J = 11.1, 2.2 Hz, 1H), 6.92 (tt, J = 8.5, 2.8 Hz, 1H), 6.42-6.34 (m, 1H), 6.32-6.26 (m, 2H), 6.17 (d, J = 3.0 Hz, 1H), 5.79 (t, J = 2.1 Hz, 1H), 5.29-5.22 (m, 1H), 4.77-4.71 (m, 1H), 4.58 (ddd, J = 8.3, 4.8, 2.0 Hz, 1H), 4.52-4.47 (m, 1H), 4.35-4.30 (m, 1H), 4.04 (ddd, J = 9.2, 5.3, 1.7 Hz, 1H), 3.98 (ddt, J = 8.7, 4.4, 2.4 Hz, 1H), 3.39 (td, J = 4.6, 2.0 Hz, 2H), 3.01 (d, J = 2.3 Hz, 3H). |
| 237 | 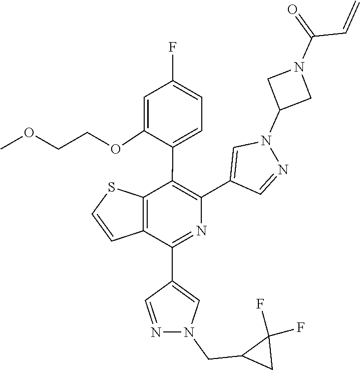 [SMILES: C=CC(=O)N1CC(n2cc(-c3nc(-c4cnn(CC5CC5(F)F)c4)c4ccsc4c3-c3ccc(F)cc3OCCOC)cn2)C1] | LCMS m/z [M + 1]: 635.1 ¹H NMR (500 MHz, CD₃OD) δ 8.49 (d, J = 5.6 Hz, 1H), 7.86-7.77 (m, 2H), 7.66 (d, J = 5.6 Hz, 1H), 7.46 (d, J = 8.0 Hz, 1H), 7.34 (dd, J = 8.3, 6.6 Hz, 1H), 7.20 (d, J = 2.3 Hz, 1H), 7.06 (dd, J = 11.2, 2.4 Hz, 1H), 6.94-6.90 (m, 1H), 6.38 (dd, J = 17.0, 10.1 Hz, 1H), 6.29 (dd, J = 17.0, 2.1 Hz, 1H), 5.78 (dd, J = 10.2, 2.1 Hz, 1H), 5.26 (ttd, J = 7.8, 5.1, 2.2 Hz, 1H), 4.76-4.71 (m, 1H), 4.60-4.54 (m, 1H), 4.52-4.39 (m, 3H), 4.33 (dd, J = 11.0, 5.3 Hz, 1H), 4.06-3.95 (m, 2H), 3.39 (t, J = 4.6 Hz, 2H), 3.01 (d, J = 1.0 Hz, 2H), 2.35 (td, J = 12.2, 7.3 Hz, 2H), 1.68 (dtd, J = 12.0, 7.6, 4.0 Hz, 1H), 1.54-1.48 (m, 1H). |
| 238 | 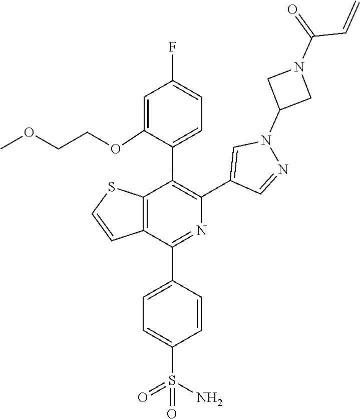 [SMILES: C=CC(=O)N1CC(n2cc(-c3nc(-c4ccc(S(N)(=O)=O)cc4)c4ccsc4c3-c3ccc(F)cc3OCCOC)cn2)C1] | LCMS m/z [M + 1]: 634.1 ¹H NMR (500 MHz, CD₃OD) δ 8.13 (t, J = 1.8 Hz, 4H), 7.77 (d, J = 5.8 Hz, 1H), 7.72-7.63 (m, 2H), 7.46 (d, J = 6.8 Hz, 1H), 7.35 (ddd, J = 8.4, 5.3, 2.0 Hz, 1H), 7.09 (dd, J = 11.0, 2.4 Hz, 1H), 6.99-6.89 (m, 1H), 6.38 (dd, J = 17.0, 10.1 Hz, 1H), 6.29 (dd, J = 17.0, 2.1 Hz, 1H), 5.79 (dd, J = 10.1, 2.1 Hz, 1H), 5.25 (s, 0H), 4.75 (t, J = 8.8 Hz, 1H), 4.58 (dd, J = 9.6, 5.2 Hz, 1H), 4.54-4.46 (m, 1H), 4.33 (dd, J = 10.9, 5.2 Hz, 1H), 4.08 (dt, J = 11.1, 4.5 Hz, 1H), 4.01 (dt, J = 10.8, 4.6 Hz, 1H), 3.42 (t, J = 4.6 Hz, 2H), 3.05 (d, J = 0.9 Hz, 3H). |
| 239 | 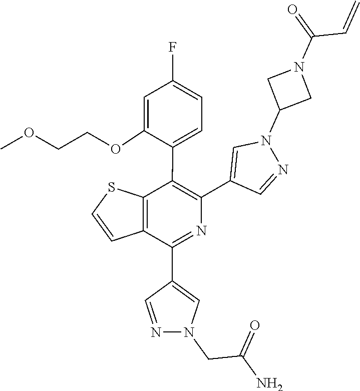 [SMILES: C=CC(=O)N1CC(n2cc(-c3nc(-c4cnn(CC(N)=O)c4)c4ccsc4c3-c3ccc(F)cc3OCCOC)cn2)C1] | LCMS m/z [M + 1]: 602.1 ¹H NMR (500 MHz, CD₃OD) δ 8.51 (s, 1H), 8.32 (s, 1H), 7.87 (dd, J = 5.7, 1.8 Hz, 1H), 7.78 (d, J = 5.8 Hz, 1H), 7.67 (dd, J = 5.6, 2.0 Hz, 1H), 7.45 (d, J = 6.7 Hz, 1H), 7.32 (ddd, J = 8.4, 6.7, 1.6 Hz, 1H), 7.06 (dd, J = 11.1, 2.4 Hz, 1H), 6.92 (td, J = 8.3, 2.3 Hz, 1H), 6.46-6.34 (m, 1H), 6.30 (dd, J = 17.1, 2.1 Hz, 1H), 5.79 (dd, J = 10.1, 2.1 Hz, 1H), 5.26 (dq, J = 10.0, 4.3, 2.3 Hz, 1H), 5.04 (s, 2H), 4.79-4.70 (m, 1H), 4.59 (dd, J = 9.7, 5.2 Hz, 1H), 4.54-4.44 (m, 1H), 4.34 (dd, J = 11.0, 5.3 Hz, 1H), 4.10-3.92 (m, 3H), 3.40 (t, J = 4.6 Hz, 2H), 3.03 (s, 3H). |
| 240 | 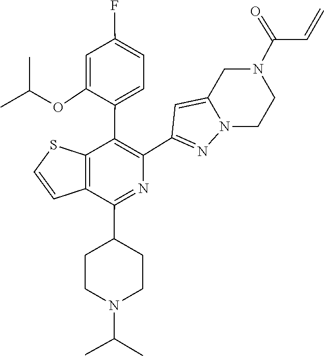 [SMILES: C=CC(=O)N1CCn2nc(-c3nc(C4CCN(C(C)C)CC4)c4ccsc4c3-c3ccc(F)cc3OC(C)C)cc2C1] | LCMS m/z [M + 1]: 588.3 ¹H NMR (400 MHz, CD₃OD) δ 7.69-7.86 (m, 2H), 7.26 (t, J = 8.0 Hz, 1H), 6.78-6.91 (m, 3H), 6.28 (d, J = 17.2 Hz, 1H), 5.76-5.84 (m, 2H), 4.75-4.82 (m, 2H), 4.45-4.49 (m, 1H), 4.19-4.23 (m, 2H), 4.11-4.13 (m, 2H), 3.77-3.85 (m, 1H), 3.59-3.64 (m, 3H), 3.31- 3.36 (m, 2H), 2.46-2.61 (m, 2H), 2.28-2.39 (m, 2H), 1.45 (d, J = 6.8 Hz, 6H), 1.05 (d, J = 6.0 Hz, 3H), 0.88 (d, J = 6.0 Hz, 3H) |
| 241 | 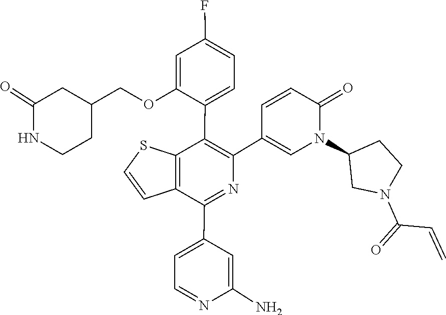 [SMILES: C=CC(=O)N1CC[C@H](n2cc(-c3nc(-c4ccnc(N)c4)c4ccsc4c3-c3ccc(F)cc3OCC3CCNC(=O)C3)ccc2=O)C1] | LCMS m/z [M + 1]: 665.3 ¹H NMR (400 MHz, CD₃OD) δ 8.26-8.30 (m, 1H), 8.01 (d, J = 6.7 Hz, 1H), 7.85-7.91 (m, 2H), 7.79-7.81 (m, 1H), 7.62 (s, 1H), 7.52 (d, J = 6.7 Hz, 1H), 7.32- 7.40 (m, 1H), 6.96-7.01 (m, 1H), 6.84-6.89 (m, 1H), 6.50-6.74 (m, 2H), 6.25-6.29 (m, 1H), 5.74 (t, J = 11.8 Hz, 1H), 5.51-5.60 (m, 1H), 3.55-3.96 (m, 6H), 3.09-3.12 (m, 2H), 2.20-2.30 (m, 2H), 1.99-2.03 (m, 2H), 1.68-1.79 (m, 1H), 1.58-1.60 (m, 1H), 1.15-1.24 (m, 1H). |
| 241a | 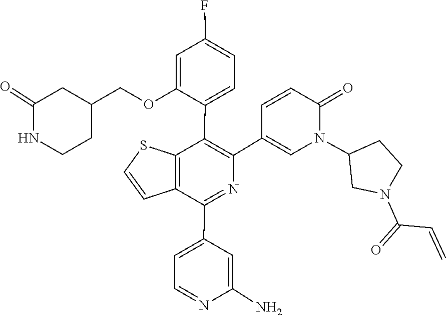 [SMILES: C=CC(=O)N1CCC(n2cc(-c3nc(-c4ccnc(N)c4)c4ccsc4c3-c3ccc(F)cc3OCC3CCNC(=O)C3)ccc2=O)C1] | |
| 242 | 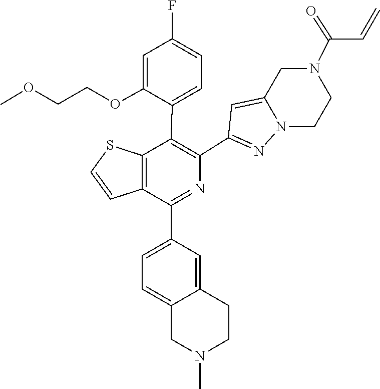 [SMILES: C=CC(=O)N1CCn2nc(-c3nc(-c4ccc5c(c4)CCN(C)C5)c4ccsc4c3-c3ccc(F)cc3OCCOC)cc2C1] | LCMS m/z [M + 1]: 624.2 ¹H NMR (400 MHz, CD₃OD) δ 7.89-7.99 (m, 3H), 7.73 (d, J = 5.6 Hz, 1H), 7.52 (d, J = 7.6 Hz, 1H), 7.32-7.40 (m, 1H), 7.01-7.09 (m, 1H), 6.68-6.96 (m, 2H), 6.28 (d, J = 16.8 Hz, 1H), 5.77-5.92 (m, 2H), 4.62-4.84 (m, 3H), 4.47-4.58 (m, 1H), 4.21-4.32 (m, 2H), 4.10-4.16 (m, 2H), 4.04-4.10 (m, 1H), 3.93-4.01 (m, 1H), 3.63-3.93 (m, 1H), 3.46-3.61 (m, 1H), 3.34- 3.46 (m, 4H), 3.13 (s, 3H), 3.08 (s, 3H). |
| 243 | 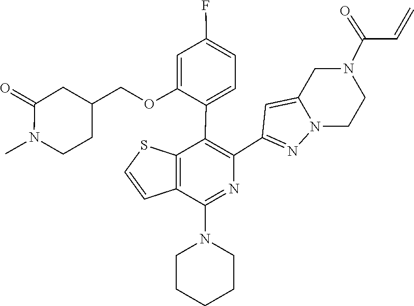 [SMILES: C=CC(=O)N1CCn2nc(-c3nc(N4CCCCC4)c4ccsc4c3-c3ccc(F)cc3OCC3CCN(C)C(=O)C3)cc2C1] | LCMS m/z [M + 1]: 629.2 ¹H NMR (400 MHz, CD₃OD) δ 7.90 (d, J = 3.3 Hz, 2H), 7.36 (t, J = 7.6 Hz, 1H), 7.05-7.12 (m, 1H), 6.70- 6.98 (m, 2H), 6.27 (d, J = 16.9 Hz, 1H), 5.77-5.87 (m, 1H), 5.53 (d, J = 9.2 Hz, 1H) 4.63-4.77 (m, 1H), 4.24- 4.46 (m, 2H), 4.07-4.18 (m, 2H), 3.93-4.01 (m, 4H), 3.72-3.92 (m, 2H), 3.08-3.22 (m, 3H), 2.78 (d, J = 19.3 Hz, 3H), 1.66-2.06 (m, 10H), 1.22-1.38 (m, 1H). |
| 244 | 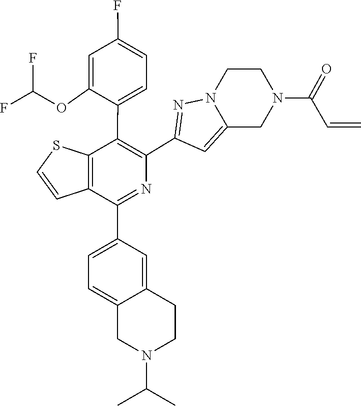 [SMILES: C=CC(=O)N1CCn2nc(-c3nc(-c4ccc5c(c4)CCN(C(C)C)C5)c4ccsc4c3-c3ccc(F)cc3OC(F)F)cc2C1] | LCMS m/z [M + 1]: 644.2 ¹H NMR (400 MHz, CD₃OD) δ 7.88-7.96 (m, 3H), 7.73 (d, J = 5.6 Hz, 1H), 7.46-7.57 (m, 2H), 7.14-7.25 (m, 2H), 6.72-6.94 (m, 2H), 7.27 (d, J = 16.7 Hz, 1H), 5.98-6.07 (m, 1H), 5.82 (d, J = 10.2 Hz, 1H), 4.84- 4.87 (m, 1H), 4.75-4.82 (m, 1H), 4.61 (s, 2H), 4.16- 4.22 (m, 2H), 4.07-4.14 (m, 2H), 3.74-3.90 (m, 2H), 3.35-3.53 (m, 3H), 1.51 (d, J = 6.6 Hz, 6H). |
| 245 | 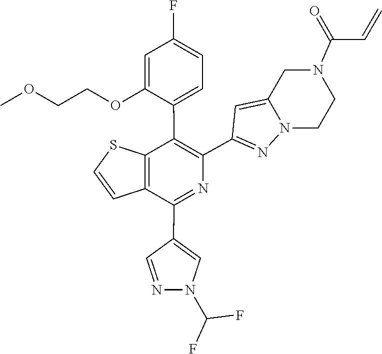 [SMILES: C=CC(=O)N1CCn2nc(-c3nc(-c4cnn(C(F)F)c4)c4ccsc4c3-c3ccc(F)cc3OCCOC)cc2C1] | LCMS m/z [M + 1]: 595.3 ¹H NMR (400 MHz, CD₃OD) δ 9.07 (s, 1H), 8.53 (s, 1H), 8.08 (d, J = 5.7 Hz, 1H), 7.99 (d, J = 5.6 Hz, 1H), 7.56-7.86 (m, 1H), 7.38 (t, J = 7.7 Hz, 1H), 7.07 (dd, J = 11.0, 1.7 Hz, 1H), 6.71-6.95 (m, 2H), 6.28 (d, J = 16.6 Hz, 1H), 5.83 (s, 2H), 4.76-4.78 (m, 2H), 4.32 (s, 2H), 3.96-4.16 (m, 4H), 3.40 (t, J = 4.4 Hz, 2H), 3.07 (s, 3H). |
| 246 | 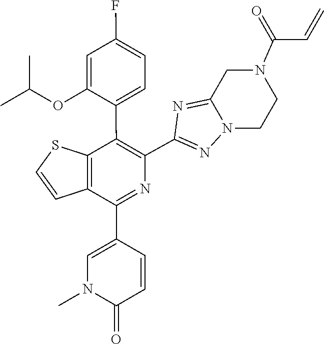 | LCMS m/z [M + 1]: 671.3 ¹H NMR (400 MHz, CD₃OD) δ8.54 (s, 1H), 8.23 (dd, J = 9.4, 2.2 Hz, 1H), 7.98 (d, J = 5.50 Hz, 1H), 7.87 (d, J = 5.60 Hz, 1H), 7.37 (dt, J = 8.0, 7.0 Hz, 1H), 6.73-6.86 (m, 4H), 6.30 (d, J = 16.7 Hz, 1H), 5.85 (dd, J = 10.7, 1.4 Hz, 1H), 4.99 (s, 2H), 4.40- 4.51 (m, 1H), 4.16 (s, 4H), 3.74 (s, 3H), 1.03 (d, J = 6.0 Hz, 3H), 0.88 (d, J = 5.9 Hz, 3H). |
| 247 | 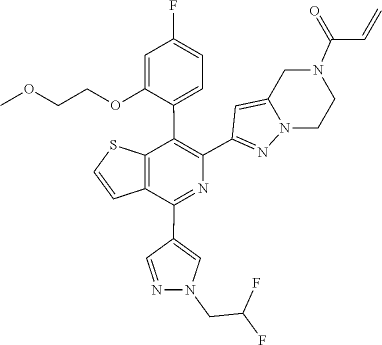 | LCMS m/z [M + 1]: 571.3 ¹H NMR (400 MHz, CD₃OD) δ 8.54 (s, 1H), 8.23 (dd, J = 9.4, 2.2 Hz, 1H), 7.98 (d, J = 5.5 Hz, 1H), 7.87 (d, J = 5.6 Hz, 1H), 7.37 (dt, J = 8.0, 7.0 Hz, 1H), 6.73-6.86 (m, 4H), 6.30 (d, J = 16.7 Hz, 1H), 5.85 (dd, J = 10.7, 1.4 Hz, 1H), 4.99 (s, 2H), 4.40-4.51 (m, 1H), 4.16 (s, 4H), 3.74 (s, 3H), 1.03 (d, J = 6.0 Hz, 3H), 0.88 (d, J = 5.9 Hz, 3H). |
| 248 | 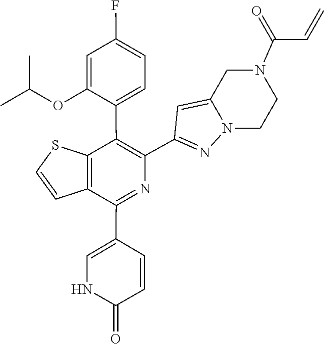 [SMILES: C=CC(=O)N1CCn2nc(-c3nc(-c4ccc(=O)[nH]c4)c4ccsc4c3-c3ccc(F)cc3OC(C)C)cc2C1] | LCMS m/z [M + 1]: 556.3 ¹H NMR (400 MHz, CD₃OD) δ 8.20-8.27 (m, 2H), 7.94 (d, J = 5.7 Hz, 1H), 7.79 (d, J = 5.6 Hz, 1H), 7.35 (t, J = 7.6 Hz, 1H), 6.94 (d, J = 11.5 Hz, 1H), 6.83- 6.87 (m, 2H), 6.75 (d, J = 10.2 Hz, 1H), 6.27 (d, J = 16.7 Hz, 1H), 5.76-5.85 (m, 2H), 4.72-4.82 (s, 2H), 4.49-4.54 (m, 1H), 4.27 (s, 2H), 4.12 (t, J = 5.5 Hz, 2H), 1.06 (d, J = 6.0 Hz, 3H), 0.89 (d, J = 5.8 Hz, 3H). |
| 249 | 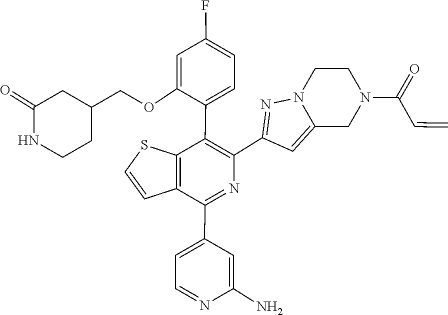 [SMILES: C=CC(=O)N1CCn2nc(-c3nc(-c4ccnc(N)c4)c4ccsc4c3-c3ccc(F)cc3OCC3CCNC(=O)C3)cc2C1] | LCMS m/z [M + 1]: 624.3 ¹H NMR (400 MHz, CD₃OD) δ8.01 (d, J = 6.6 Hz, 1H), 7.82-7.88 (m, 1H), 7.77 (t, J = 5.1 Hz, 1H), 7.62 (s, 1H), 7.56 (d, J = 6.5 Hz, 1H), 7.34 (dd, J = 14.5, 6.9 Hz, 1H), 6.99 (d, J = 10.9 Hz, 1H), 6.71-6.93 (m, 2H), 6.28 (d, J = 17.2 Hz, 1H), 5.88-5.98 (m, 1H), 5.82 (d, J = 10.5 Hz, 1H), 4.72-4.82 (m, 2H), 4.10-4.25 (m, 4H), 3.71-3.91 (m, 2H), 2.82-3.16 (m, 2H), 1.86-2.12 (m, 2H), 1.51-1.82 (m, 2H), 1.08-1.22 (m, 1H). |
| 250 | 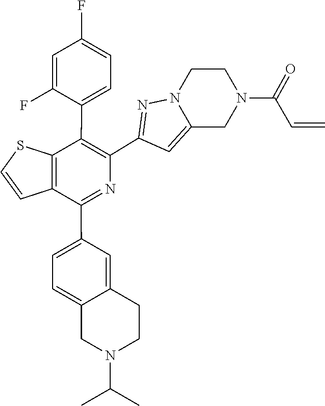 [SMILES: C=CC(=O)N1CCn2nc(-c3nc(-c4ccc5c(c4)CCN(C(C)C)C5)c4ccsc4c3-c3ccc(F)cc3F)cc2C1] | LCMS m/z [M + 1]: 596.3 ¹H NMR (400 MHz, CD₃OD) δ 7.87-7.95 (m, 2H), 7.83 (d, J = 5.6 Hz, 1H), 7.69 (d, J = 5.6 Hz, 1H), 7.44-7.55 (m, 2H), 7.08-7.19 (m, 2H), 6.66-7.02 (m, 1H), 6.28 (d, J = 16.3 Hz, 1H), 6.03-6.16 (m, 1H), 5.76-5.86 (m, 1H), 4.80 (s, 2H), 4.60 (s, 2H), 4.05- 4.22 (m, 4H), 3.73-3.89 (m, 2H), 3.34-3.58 (m, 3H), 1.51 (d, J = 6.4 Hz, 6H). |
| 251 | 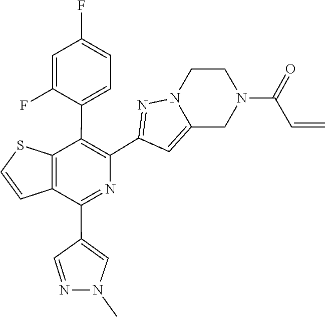 | LCMS m/z [M + 1]: 565.3 ¹H NMR (400 MHz, CD₃OD) δ8.60 (s, 1H), 8.32 (s, 1H), 8.05 (s, 2H), 7.51-7.62 (m, 1H), 7.14-7.25 (m, 2H), 6.68-6.94 (m, 1H), 6.28 (d, J = 17.2 Hz, 1H), 5.93-6.03 (m, 1H), 5.77-5.87 (m, 1H), 4.80 (s, 2H), 4.20-4.31 (m, 2H), 4.11-4.18 (m, 2H), 4.08 (s, 3H). |
| 252 | 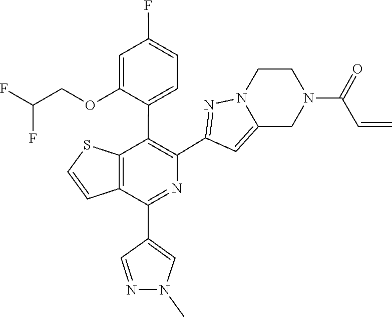 | LCMS m/z [M + 1]: 653.3 ¹H NMR (400 MHz, CD₃OD) δ 8.67 (s, 1H), 8.35 (s, 1H), 8.11 (d, J = 5.6 Hz, 1H), 8.09 (d, J = 5.6 Hz, 1H), 7.46 (t, J = 7.4 Hz, 1H), 7.14 (d, J = 10.0 Hz, 1H), 6.99-7.04 (m, 1H), 6.76-6.92 (m, 1H), 6.29 (d, J = 16.6 Hz, 1H), 5.65-5.94 (m, 3H), 4.77-4.86 (m, 2H), 4.28-4.37 (m, 2H), 4.12-4.26 (m, 4H), 4.11 (s, 3H). |
| 253 | 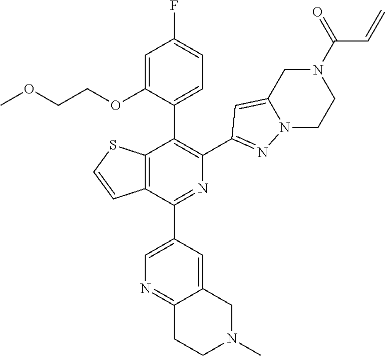 [SMILES: C=CC(=O)N1CCn2nc(-c3nc(-c4cnc5c(c4)CN(C)CC5)c4ccsc4c3-c3ccc(F)cc3OCCOC)cc2C1] | LCMS m/z [M + 1]: 625.2 ¹H NMR (400 MHz, CD₃OD) δ 9.13 (s, 1H), 8.34 (s, 1H), 7.87 (d, J = 5.6 Hz, 1H), 7.72 (d, J = 5.6 Hz, 1H), 7.32 (t, J = 7.9 Hz, 1H), 6.96-7.05 (m, 1H), 6.69- 6.92 (m, 2H), 6.27 (d, J = 16.8 Hz, 1H), 5.74-5.94 (m, 2H), 4.62-4.85 (m, 4H), 4.17-4.28 (m, 2H), 4.09-4.16 (m, 2H), 4.00-4.09 (m, 1H), 3.89-3.97 (m, 1H), 3.73- 3.87 (m, 2H), 3.45 (t, J = 6.0 Hz, 2H), 3.39 (t, J = 4.7 Hz, 2H), 3.16 (s, 3H), 3.06 (s, 3H), 2.65 (s, 1H). |
| 254 | 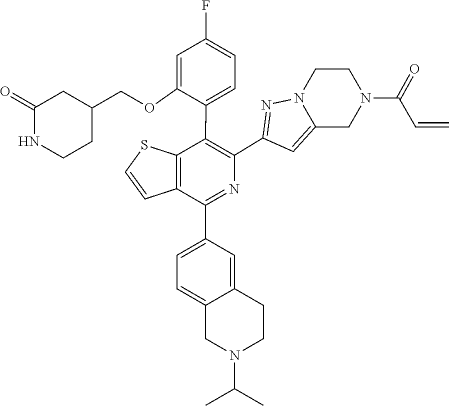 [SMILES: C=CC(=O)N1CCn2nc(-c3nc(-c4ccc5c(c4)CCN(C(C)C)C5)c4ccsc4c3-c3ccc(F)cc3OCC3CCNC(=O)C3)cc2C1] | LCMS m/z [M + 1]: 705.4 ¹H NMR (400 MHz, CD₃OD) δ 7.91-8.03 (m, 3H), 7.74 (d, J = 4.1 Hz, 1H), 7.57 (d, J = 7.5 Hz, 1H), 7.39-7.47 (m, 1H), 7.06 (d, J = 10.8 Hz, 1H), 6.69- 6.99 (m, 2H), 6.28 (d, J = 17.0 Hz, 1H), 5.80-5.85 (m, 2H), 4.73-4.86 (m, 2H), 4.63 (s, 2H), 4.08-4.35 (m, 4H), 3.73-3.97 (m, 4H), 3.37-3.58 (m, 3H), 3.02-3.18 (m, 2H), 1.96-2.15 (m, 2H), 1.56-1.79 (m, 2H), 1.49 (dd, J = 23.0, 6.6 Hz, 6H), 1.17-1.28 (m, 1H). |
| 255 | 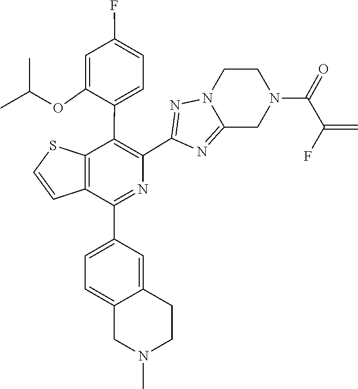 [SMILES: C=C(F)C(=O)N1CCn2nc(-c3nc(-c4ccc5c(c4)CCN(C)C5)c4ccsc4c3-c3ccc(F)cc3OC(C)C)nc2C1] | LCMS m/z [M + 1]: 627.2 ¹H NMR (400 MHz, CD₃OD) δ 7.96 (s, 1H), 7.93 (d, J = 8.2 Hz, 1H), 7.86 (d, J = 5.6 Hz, 1H), 7.71 (d, J = 5.6 Hz, 1H), 7.44 (d, J = 8.0 Hz, 1H), 7.36 (t, J = 7.2 Hz, 1H), 6.75-6.84 (m, 2H), 5.36-5.45 (m, 1H), 5.32 (s, 1H), 4.92 (s, 2H), 4.67-4.71 (m, 1H), 4.42-4.48 (m, 2H), 4.13-4.18 (m, 4H), 3.83-3.84 (m, 1H), 3.47- 3.51 (m, 1H), 3.35-3.38 (m, 2H), 3.12 (s, 3H), 1.05 (d, J = 6.0 Hz, 3H), 0.89 (d, J = 5.9 Hz, 3H). |
| 256 | 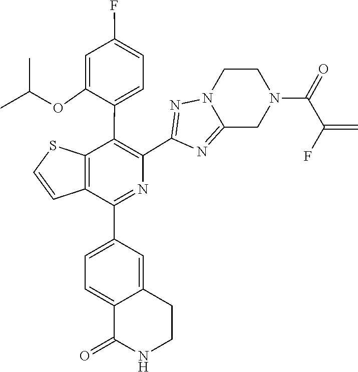 [SMILES: C=C(F)C(=O)N1CCn2nc(-c3nc(-c4ccc5c(c4)CCNC5=O)c4ccsc4c3-c3ccc(F)cc3OC(C)C)nc2C1] | LCMS m/z [M + 1]: 627.2 ¹H NMR (400 MHz, CD₃OD) δ 8.14 (d, J = 8.4 Hz, 1H), 7.98 (d, J = 4.1 Hz, 2H), 7.84 (d, J = 5.6 Hz, 1H), 7.73 (d, J = 5.6 Hz, 1H), 7.37 (t, J = 7.8 Hz, 1H), 6.74-6.83 (m, 2H), 5.35-5.45 (m, 1H), 5.32 (s, 1H), 4.41-4.47 (m, 1H), 4.11-4.17 (m, 4H), 3.59 (t, J = 6.6 Hz, 2H), 3.14 (t, J = 6.6 Hz, 3H), 1.19-1.32 (m, 1H), 1.05 (d, J = 6.0 Hz, 3H), 0.89 (d, J = 6.0 Hz, 3H). |
| 257 | 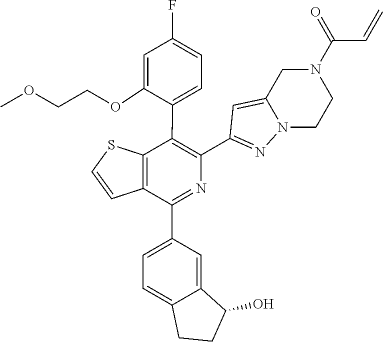 [SMILES: C=CC(=O)N1CCn2nc(-c3nc(-c4ccc5c(c4)[C@H](O)CC5)c4ccsc4c3-c3ccc(F)cc3OCCOC)cc2C1] | LCMS m/z [M + 1]: 611.2 ¹H NMR (400 MHz, CD₃OD) δ 8.16 (d, J = 5.6 Hz, 1H), 7.97 (s, 1H), 7.88 (t, J = 5.3 Hz, 2H), 7.64 (d, J = 7.8 Hz, 1H), 7.43-7.47 (m, 1H), 7.12 (dd, J = 11.0, 2.0 Hz, 1H), 6.73-6.70 (m, 2H), 6.28 (d, J = 16.7 Hz, 1H), 5.81-5.85 (m, 2H), 5.36 (t, J = 6.5 Hz, 1H), 4.71- 4.86 (m, 2H), 4.00-4.34 (m, 6H), 3.43 (t, J = 4.3 Hz, 2H), 3.18-3.27 (m, 1H), 3.09 (s, 3H), 2.96-3.04 (m, 1H), 2.55-2.63 (m, 1H), 2.02-2.11 (m, 1H). |
| 258 | 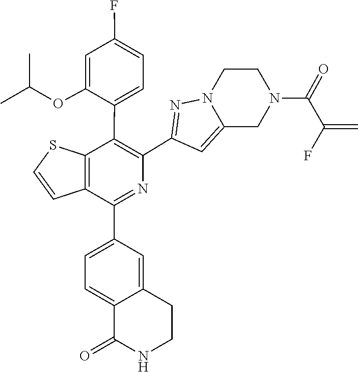 [SMILES: C=C(F)C(=O)N1CCn2nc(-c3nc(-c4ccc5c(c4)CCNC5=O)c4ccsc4c3-c3ccc(F)cc3OC(C)C)cc2C1] | LCMS m/z [M + 1]: 626.2 ¹H NMR (500 MHz, CD₃OD) δ8.24 (d, J = 7.9 Hz, 1H), 8.08 (d, J = 5.6 Hz, 1H), 7.97-8.01 (m, 2H), 7.82 (d, J = 5.6 Hz, 1H), 7.41 (t, J = 7.7 Hz, 1H), 7.00 (d = 10.1 Hz, 1H), 6.89 (t, J = 6.4 Hz, 1H), 5.84 (s, 1H), 5.28-5.41 (m, 2H), 4.77 (s, 2H), 4.54- 4.60 (m, 1H), 4.33 (t, J = 5.6 Hz, 2H), 4.01 (t, J = 5.2 Hz, 2H), 3.62 (t, J = 6.6 Hz, 2H), 3.19 (t, J = 6.5 Hz, 2H), 1.10 (d, J = 6.0 Hz, 3H), 0.93 (d, J = 6.0 Hz, 3H). |
| 259 | 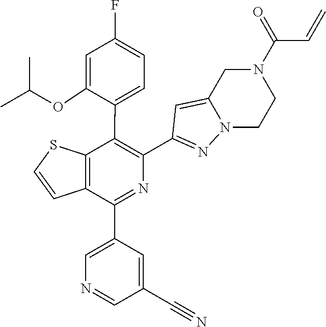 [SMILES: C=CC(=O)N1CCn2nc(-c3nc(-c4cncc(C#N)c4)c4ccsc4c3-c3ccc(F)cc3OC(C)C)cc2C1] | LCMS m/z [M + 1]: 565.3 ¹H NMR (400 MHz, CD₃OD) δ 9.43 (d, J = 2.1 Hz, 1H), 9.10 (d, J = 1.9 Hz, 1H), 8.89 (t, J = 2.0 Hz, 1H), 7.92 (d, J = 5.5 Hz, 1H), 7.76 (d, J = 5.6 Hz, 1H), 7.35 (t, J = 8.2 Hz, 1H), 6.94 (dd, J = 11.4, 2.2 Hz, 1H), 6.71-6.87 (m, 2H), 6.28 (d, J = 16.9 Hz, 1H), 5.78- 5.85 (m, 2H), 4.80 (m, 2H), 4.47-4.55 (m, 1H), 4.20- 4.30 (m, 2H), 4.12 (t, J = 5.8 Hz, 2H), 1.07 (d, J = 6.0 Hz, 3H), 0.89 (d, J = 6.0 Hz, 3H). |
| 260 | 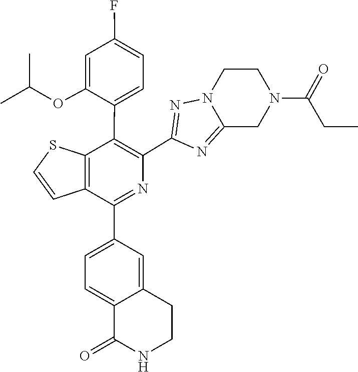 [SMILES: CCC(=O)N1CCn2nc(-c3nc(-c4ccc5c(c4)CCNC5=O)c4ccsc4c3-c3ccc(F)cc3OC(C)C)nc2C1] | LCMS m/z [M + 1]: 611.4 ¹H NMR (400 MHz, CD₃OD) δ 8.17 (d, J = 7.8 Hz, 1H), 7.96-8.01 (m, 3H), 7.79 (d, J = 5.6 Hz, 1H), 7.39 (t, J = 7.8 Hz, 1H), 6.87 (dd, J = 11.4, 1.8 Hz, 1H), 6.81 (t, J = 8.1 Hz, 1H), 4.84-4.89 (m, 2H), 4.46-4.52 (m, 1H), 4.04-4.17 (m, 4H), 3.60 (t, J = 6.6 Hz, 2H), 3.15 (t, J = 6.5 Hz, 2H), 2.46-2.57 (m, 2H), 1.10-1.16 (m, 3H), 1.06 (d, J = 6.0 Hz, 3H), 0.91 (d, J = 5.7 Hz, 3H). |
| 261 | 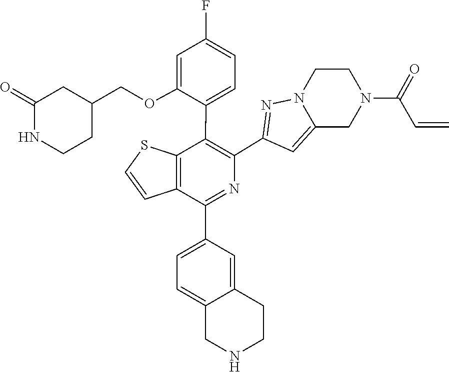 [SMILES: C=CC(=O)N1CCn2nc(-c3nc(-c4ccc5c(c4)CCNC5)c4ccsc4c3-c3ccc(F)cc3OCC3CCNC(=O)C3)cc2C1] | LCMS m/z [M + 1]: 663.4 ¹H NMR (400 MHz, CD₃OD) δ 7.91-8.02 (m, 3H), 7.75 (d, J = 5.6 Hz, 1H), 7.55 (d, J = 7.3 Hz, 1H), 7.39-7.47 (m, 1H), 7.07 (d, J = 10.7 Hz, 1H), 6.72- 3.98 (m, 2H), 6.28 (d, J = 16.4 Hz, 1H), 5.75-5.86 (m, 2H), 4.72-4.83 (m, 2H), 4.54 (s, 1H), 4.12-4.45 (m, 4H), 3.81-3.93 (m, 2H), 3.62 (t, J = 6.2 Hz, 1H), 3.02- 3.16 (m, 2H), 1.92-2.05 (m, 2H), 1.55-1.79 (m, 2H), 1.16-1.28 (m, 1H). |
| 262 | 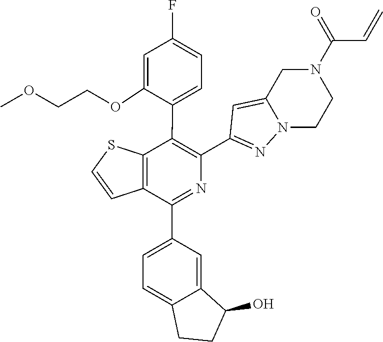 [SMILES: C=CC(=O)N1CCn2nc(-c3nc(-c4ccc5c(c4)[C@@H](O)CC5)c4ccsc4c3-c3ccc(F)cc3OCCOC)cc2C1] | LCMS m/z [M + 1]: 611.2 ¹H NMR (400 MHz, CD₃OD) δ 8.14 (d, J = 5.6 Hz, 1H), 7.90 (s, 1H), 7.86-7.88 (m, 2H), 7.63 (d, J = 8.0 Hz, 1H), 7.42-7.45 (m, 1H), 7.11 (d, J = 11.2 Hz, 1H), 6.96 (t, J = 8.4 Hz, 1H), 6.76-6.91 (m, 1H), 6.28 (d, J = 16.8 Hz, 1H), 5.83 (m, 2H), 5.36 (t, J = 6.4 Hz, 1H),4.71-4.78 (m, 1H), 4.32 (s, 2H), 4.11-4.18 (m, 3H), 3.99-4.08 (m, 2H), 3.42 (t, J = 8.4 Hz, 1H) ), 3.17-3.32 (m, 1H), 3.09 (s, 3H), 2.95-3.03 (m, 1H), 2.55-2.63 (m, 1H), 2.01-2.10 (m, 1H). |
| 263 | 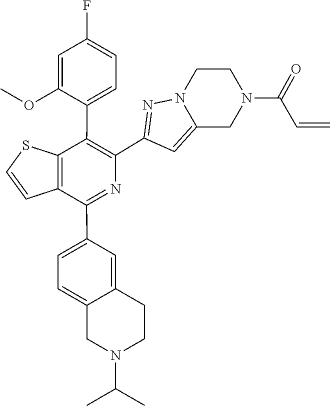 [SMILES: C=CC(=O)N1CCn2nc(-c3nc(-c4ccc5c(c4)CCN(C(C)C)C5)c4ccsc4c3-c3ccc(F)cc3OC)cc2C1] | LCMS m/z [M + 1]: 608.2 ¹H NMR (400 MHz, CD₃OD) δ 7.99 (d, J = 5.6 Hz, 1H), 7.88-7.95 (m, 2H), 7.73 (d, J = 5.2 Hz, 1H), 7.57 (d, J = 8.4 Hz, 1H), 7.35 (t, J = 7.2 Hz, 1H), 7.03 (d, J = 10.8 Hz, 1H), 6.71-6.95 (m, 2H), 6.28 (d, J = 16.4 Hz, 1H), 5.76-5.88 (m, 2H), 4.76 (s, 1H), 4.63 (s, 2H), 4.22-4.33 (m, 2H), 4.09-4.18 (m, 2H), 3.74-3.91 (m, 2H), 3.66 (s, 3H), 3.35-3.57 (m, 4H), 1.52 (d, J = 6.4 Hz, 6H). |
| 264 | 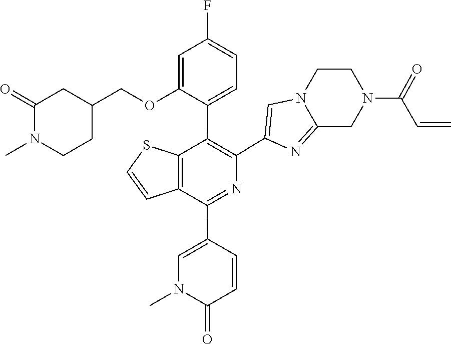 [SMILES: C=CC(=O)N1CCn2cc(-c3nc(-c4ccc(=O)n(C)c4)c4ccsc4c3-c3ccc(F)cc3OCC3CCN(C)C(=O)C3)nc2C1] | LCMS m/z [M + 1]: 653.3 ¹H NMR (400 MHz, CD₃OD) δ 8.49 (s, 1H), 8.33 (d, J = 9.2 Hz, 1H), 7.86-7.90 (m, 2H), 7.40 (dd, J = 15.1, 8.4 Hz, 1H), 7.01-7.17 (m, 2H), 6.75-6.94 (m, 2H), 6.47 (d, J = 4.7 Hz, 1H), 6.34 (d, J = 16.6 Hz, 1H), 5.88 (d, J = 11.0 Hz, 1H), 5.17-5.27 (m, 2H), 4.05- 4.20 (m, 4H), 3.91-4.01 (m, 1H), 3.72-3.88 (m, 4H), 3.06-3.20 (m, 2H), 2.72-2.79 (m, 3H), 1.87-2.32 (m, 2H), 1.54-1.72 (m, 2H). |
| 265 | 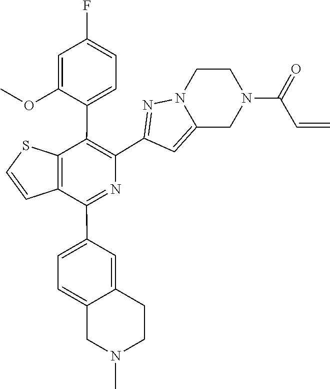 [SMILES: C=CC(=O)N1CCn2nc(-c3nc(-c4ccc5c(c4)CCN(C)C5)c4ccsc4c3-c3ccc(F)cc3OC)cc2C1] | LCMS m/z [M + 1]: 580.2 ¹H NMR (400 MHz, CD₃OD) δ 8.06 (d, J = 5.6 Hz, 1H), 7.88-7.96 (m, 2H), 7.77 (d, J = 5.6 Hz, 1H), 7.56 (d, J = 8.0 Hz, 1H), 7.38 (t, J = 7.6 Hz, 1H), 7.01-7.07 (m, 1H), 6.69-6.98 (m, 2H), 6.28 (d, J = 16.4 Hz, 1H), 5.75-5.89 (m, 2H), 4.67-4.87 (m, 3H), 4.41-4.66 (m, 1H), 4.22-4.36 (m, 2H), 4.14 (t, J = 5.4 Hz, 2H), 3.72- 4.06 (m, 1H), 3.67 (s, 3H), 3.33-3.63 (m, 3H), 3.13 (s, 3H). |
| 266 | 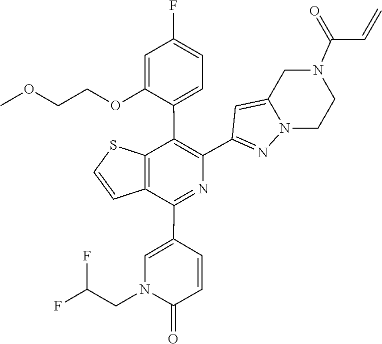 [SMILES: C=CC(=O)N1CCn2nc(-c3nc(-c4ccc(=O)n(CC(F)F)c4)c4ccsc4c3-c3ccc(F)cc3OCCOC)cc2C1] | LCMS m/z [M + 1]: 636.2 ¹H NMR (400 MHz, CD₃OD) δ 8.49 (d, J = 2.0 Hz, 1H), 8.19 (dd, J = 9.2, 2.4 Hz, 1H), 8.01 (d, J = 5.6 Hz, 1H), 7.82 (d, J = 5.6 Hz, 1H), 7.31-7.40 (m, 1H), 7.05 (dd, J = 11.2, 2.0 Hz, 1H), 6.87-6.95 (m, 1H), 6.69-6.87 (m, 2H), 6.15-6.49 (m, 2H), 5.83 (m, 2H), 4.68-4.87 (m, 2H), 4.52-4.65 (m, 2H), 4.23-4.34 (m, 2H), 4.11-4.20 (m, 2H), 4.02-4.10 (m, 1H), 3.91-4.00 (m, 1H), 3.40 (t, J = 4.4 Hz, 2H), 3.07 (s, 3H). |
| 267 | 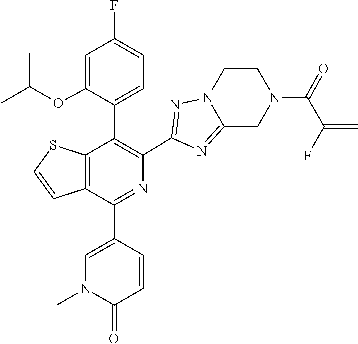 [SMILES: C=C(F)C(=O)N1CCn2nc(-c3nc(-c4ccc(=O)n(C)c4)c4ccsc4c3-c3ccc(F)cc3OC(C)C)nc2C1] | LCMS m/z [M + 1]: 589.2 ¹H NMR (400 MHz, CD₃OD) δ 8.53 (d, J = 1.8 Hz, 1H), 8.23 (dd, J = 9.3, 2.2 Hz, 1H), 7.95 (d, J = 5.6 Hz, 1H), 7.85 (d, J = 5.6 Hz, 1H), 7.35 (t, J = 8.1 Hz, 1H), 6.74-6.88 (m, 3H), 5.33-5.46 (m, 2H), 4.95 (s, 2H), 4.43-4.49 (m, 1H), 4.12-4.19 (m, 4H), 3.73 (m, 3H), 1.03 (d, J = 6.0 Hz, 3H), 0.89 (d, J = 6.0 Hz, 3H). |
| 268 | 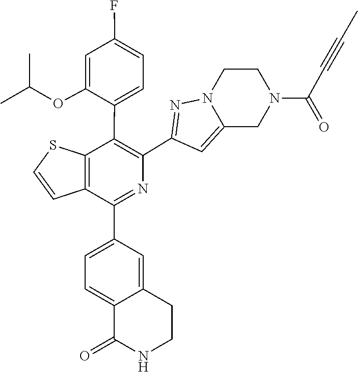 [SMILES: CC#CC(=O)N1CCn2nc(-c3nc(-c4ccc5c(c4)CCNC5=O)c4ccsc4c3-c3ccc(F)cc3OC(C)C)cc2C1] | LCMS m/z [M + 1]: 620.2 ¹H NMR (400 MHz, CD₃OD) δ 8.25 (d, J = 7.8 Hz, 1H), 8.11 (d, J = 5.6 Hz, 1H), 7.99 (d, J = 10.9 Hz, 2H), 7.83 (d, J = 5.6 Hz, 1H), 7.40-7.45 (m, 1H), 7.00-7.04 (m, 1H), 6.88-6.93 (m, 1H), 5.83 (d, J = 8.2 Hz, 1H), 4.95 (s, 1H), 4.72 (s, 1H), 4.56-4.60 (m, 1H), 4.10-4.34 (m, 4H), 3.62 (t, J = 13.2 Hz, 2H), 3.19 (t, J = 6.6 Hz, 2H), 2.06 (d, J = 9.8 Hz, 3H), 1.11 (d, J = 5.9 Hz, 3H), 0.94 (t, J = 4.4 Hz, 3H). |
| 269 | 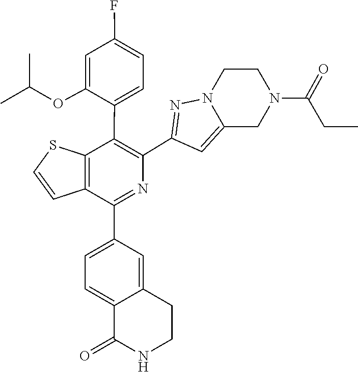 [SMILES: CCC(=O)N1CCn2nc(-c3nc(-c4ccc5c(c4)CCNC5=O)c4ccsc4c3-c3ccc(F)cc3OC(C)C)cc2C1] | LCMS m/z [M + 1]: 610.2 ¹H NMR (400 MHz, CD₃OD) δ 8.25 (d, J = 7.8 Hz, 1H), 8.11 (d, J = 5.6 Hz, 1H), 7.99 (d, J = 11.2 Hz, 2H), 7.83 (d, J = 5.6 Hz, 1H), 7.40-7.45 (m, 1H), 7.02 (d, J = 11.0 Hz, 1H), 6.90 (t, J = 8.2 Hz, 1H), 5.80 (s, 1H), 4.69-474 (m, 2H), 4.55-4.61 (m, 1H), 4.24-4.23 (m, 2H), 4.02-4.08 (m, 2H), 3.62 (t, J = 6.4 Hz, 2H), 3.19 (t, J = 6.4 Hz, 2H), 2.41-2.57 (m, 2H), 1.08-1.16 (m, 6H), 0.93 (d, J = 5.7 Hz, 3H). |
| 270 | 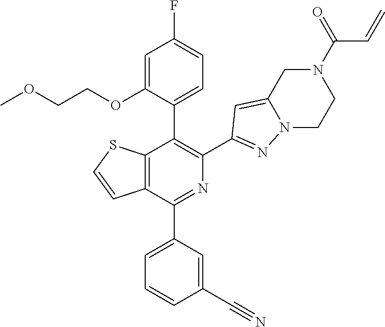 [SMILES: C=CC(=O)N1CCn2nc(-c3nc(-c4cccc(C#N)c4)c4ccsc4c3-c3ccc(F)cc3OCCOC)cc2C1] | LCMS m/z [M + 1]: 580.3 ¹H NMR (400 MHz, CD₃OD) δ 8.41 (s, 1H), 8.28 (d, J = 7.8 Hz, 1H), 8.00 (t, J = 6.1 Hz, 2H), 7.86 (t, J = 7.8 Hz, 1H), 7.72 (d, J = 5.6 Hz, 1H), 7.38 (t, J = 7.4 Hz, 1H), 7.06 (d, J = 11.0 Hz, 1H), 6.73-6.95 (m, 2H), 6.28 (d, J = 16.7 Hz, 1H), 5.75-5.85 (m, 2H), 4.70- 4.87 (m, 1H), 4.26 (s, 2H), 4.05-4.17(m, 3H), 3.93- 4.01 (m, 1H), 3.40 (t, J = 4.4 Hz, 2H), 3.08 (s, 3H). |
| 271 | 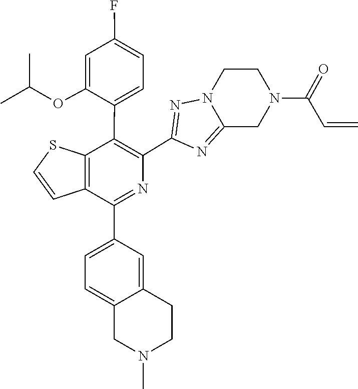 [SMILES: C=CC(=O)N1CCn2nc(-c3nc(-c4ccc5c(c4)CCN(C)C5)c4ccsc4c3-c3ccc(F)cc3OC(C)C)nc2C1] | LCMS m/z [M + 1]: 609.4 ¹H NMR (400 MHz, CD₃OD) δ 7.92-7.96 (m, 2H), 7.88 (d, J = 5.6 Hz, 1H), 7.72 (d, J = 5.60 Hz, 1H), 7.45 (d, J = 7.94 Hz, 1H), 7.37 (t, J = 7.72 Hz, 1H), 6.76-6.84 (m, 3H), 6.30 (d, J = 16.6 Hz, 1H), 5.85 (d, J = 11.0 Hz, 1H), 4.88-4.95 (m, 2H), 4.68-4.72 (m, 1H), 4.42-4.48 (m, 2H), 4.12-4.18 (m, 4H), 3.84-3.87 (m, 1H), 3.35-3.52 (m, 3H), 3.12 (s, 3H), 1.05 (d, J = 6.0 Hz, 3H), 0.89 (d, J = 5.9 Hz, 3H). |
| 272 | 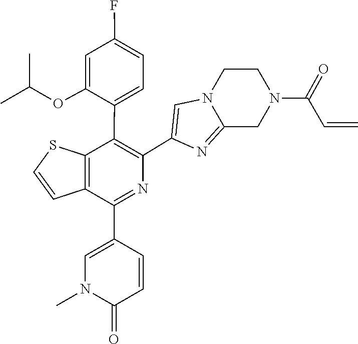 [SMILES: C=CC(=O)N1CCn2cc(-c3nc(-c4ccc(=O)n(C)c4)c4ccsc4c3-c3ccc(F)cc3OC(C)C)nc2C1] | LCMS m/z [M + 1]: 570.2 ¹H NMR (400 MHz, CD₃OD) δ 8.48 (s, 1H), 8.33 (dd, J = 9.4, 1.8 Hz, 1H), 7.85-7.91 (m, 2H), 7.34 (t, J = 7.6 Hz, 1H), 7.11 (d, J = 10.1 Hz, 1H), 6.76-6.98 (m, 3H), 6.46 (s, 1H), 6.34 (d, J = 16.9 Hz, 1H), 5.88 (d, J = 11.0 Hz, 1H), 5.10-5.24 (m, 2H), 4.57-4.63 (m, 1H), 4.09-4.25 (m, 4H), 3.76 (s, 3H), 1.06 (d, J = 6.0 Hz, 3H), 1.01 (d, J = 6.0 Hz, 3H) |
| 273 | 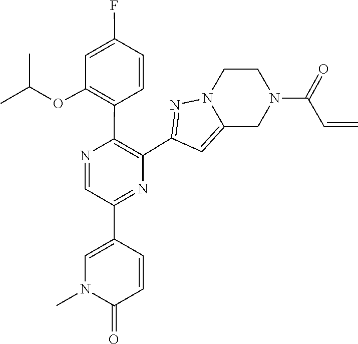 [SMILES: C=CC(=O)N1CCn2nc(-c3nc(-c4ccc(=O)n(C)c4)cnc3-c3ccc(F)cc3OC(C)C)cc2C1] | LCMS m/z [M + 1]: 515.3 ¹H NMR (400 MHz, CD₃OD) δ 8.94 (s, 1H), 8.78- 8.81 (m, 1H), 8.40-8.45 (m, 1H), 7.42-7.48 (m, 1H), 6.66-6.90 (m, 4H), 6.28 (d, J = 16.8 Hz, 1H), 5.99 (s, 1H), 5.82 (d, J = 9.9 Hz, 1H), 4.77 (s, 1H), 4.34-4.44 (m, 1H), 4.17-4.26 (m, 2H), 4.08-4.16 (m, 2H), 3.71 (s, 3H), 0.73-1.14 (m, 6H). |
| 274 | 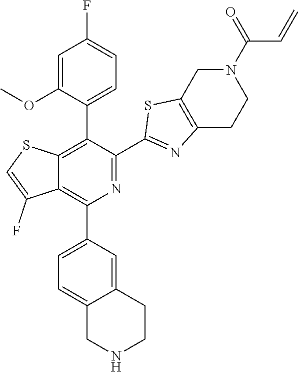 [SMILES: C=CC(=O)N1CCc2nc(-c3nc(-c4ccc5c(c4)CCNC5)c4c(F)csc4c3-c3ccc(F)cc3OC)sc2C1] | LCMS m/z [M + 1]: 601.2 ¹H NMR (400 MHz, CD₃OD) δ 7.70-7.71 (m, 2H), 7.37-7.41 (m, 2H), 7.30 (t, J = 8.4 Hz, 1H), 6,93 (d, J = 10.8 Hz, 1H), 6.74-6.88 (m, 2H), 6.22-6.26 (m, 1H), 5.76-5.80 (m, 1H), 4.84 (s, 2H), 4.49 (s, 2H), 3.90-3.91 (m, 2H), 3.58-3.60 (m, 5H), 3.25-3.26 (m, 2H), 2.70-2.71 (m, 2H). |
| 275 | 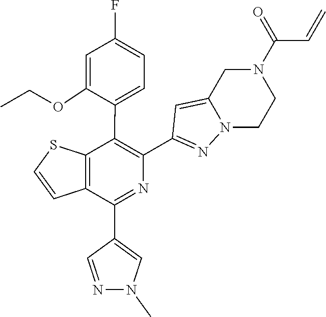 [SMILES: C=CC(=O)N1CCn2nc(-c3nc(-c4cnn(C)c4)c4ccsc4c3-c3ccc(F)cc3OCC)cc2C1] | LCMS m/z [M + 1]: 529.3 ¹H NMR (400 MHz, CD₃OD) δ 8.67 (s, 1H), 8.35 (s, 1H), 8.15 (d, J = 5.6 Hz, 1H), 8.08 (d, J = 5.6 Hz, 1H), 7.40 (t, J = 7.4 Hz, 1H), 7.04 (d, J = 11.4 Hz, 1H), 6.74-6.94 (m, 2H), 6.49 (d, J = 16.6 Hz, 1H), 5.78-5.83 (m, 2H), 4.77-4.85 (m, 2H), 4.31-4.38 (m, 2H), 4.14-4.17 (m, 2H), 4.11 (s, 3H), 3.86-4.05 (m, 2H), 1.04 (t, J = 7.2 Hz, 3H). |
| 276 | 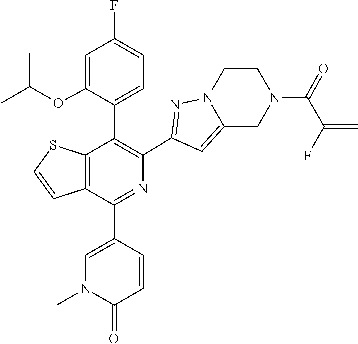 [SMILES: C=C(F)C(=O)N1CCn2nc(-c3nc(-c4ccc(=O)n(C)c4)c4ccsc4c3-c3ccc(F)cc3OC(C)C)cc2C1] | LCMS m/z [M + 1]: 588.3 ¹H NMR (400 MHz, CD₃OD) δ 8.57 (d, J = 2.0 Hz, 1H), 8.10-8.14 (m, 2H), 7.94 (d, J = 5.7 Hz, 1H), 7.39 (t, J = 6.8 Hz, 1H), 7.01 (d, J = 9.7 Hz, 1H), 6.89 (t, J = 8.3 Hz, 1H), 6.79 (d, J = 9.5 Hz, 1H), 5.80 (s, 1H), 5.25-5.42 (m, 2H), 4.77 (s, 2H), 4.49-4.62 (m, 1H), 4.31-4.39 (m, 2H), 4.09-4.14 (m, 2H), 3.75 (s, 3H), 1.08 (d, J = 6.0 Hz, 3H), 0.91 (d, J = 5.9 Hz, 3H). |
| 277 | 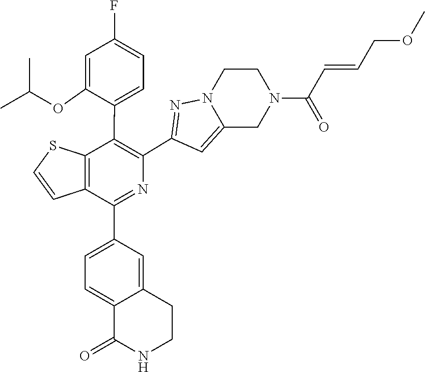 [SMILES: COC/C=C/C(=O)N1CCn2nc(-c3nc(-c4ccc5c(c4)CCNC5=O)c4ccsc4c3-c3ccc(F)cc3OC(C)C)cc2C1] | LCMS m/z [M + 1]: 652.2 ¹H NMR (400 MHz, CD₃OD) δ 8.25 (d, J = 7.8 Hz, 1H), 8.11 (d, J = 5.6 Hz, 1H), 7.98 (d, J = 11.3 Hz, 2H), 7.83 (d, J = 5.7 Hz, 1H), 7.42 (t, J = 6.8 Hz, 1H), 7.02 (d, J = 11.4 Hz, 1H), 6.84-6.92 (m, 2H), 6.66- 6.75 (m, 1H), 5.80 (s, 1H), 4.75 (s, 2H), 4.54-4.60 (m, 1H), 4.30 (s, 2H), 4.12 (s, 4H), 3.60-3.63 (m, 2H), 3.40 (s, 3H), 3.17-3.20 (m, 2H), 1.10 (d, J = 5.8 Hz, 3H), 0.92 (d, J = 5.9 Hz, 3H). |
| 278 | 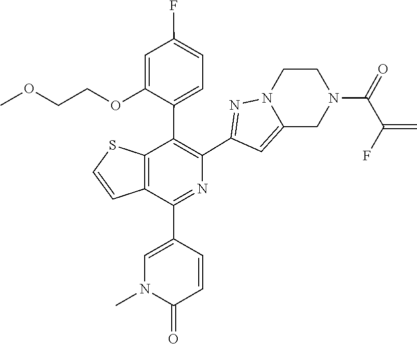 [SMILES: C=C(F)C(=O)N1CCn2nc(-c3nc(-c4ccc(=O)n(C)c4)c4ccsc4c3-c3ccc(F)cc3OCCOC)cc2C1] | LCMS m/z [M + 1]: 604.3 ¹H NMR (400 MHz, CD₃OD) δ 8.55 (d, J = 2.1 Hz, 1H), 8.09-8.14 (m, 2H), 7.94 (d, J = 5.6 Hz, 1H), 7.38 (t, J = 8.4 Hz, 1H), 7.09 (dd, J = 10.9, 1.8 Hz, 1H), 6.90- 6.96 (m, 1H), 6.79 (d, J = 9.4 Hz, 1H), 5.84 (s, 1H), 5.29-5.41 (m, 2H), 4.73 (s, 2H), 4.33-4.35 (m, 2H), 4.10-4.13 (m, 2H), 4.06-4.08 (m, 1H), 3.95-4.02 (m, 1H), 3.75 (s, 3H), 3.41 (t, J = 4.6 Hz, 2H), 3.09 (s, 3H). |
| 279 | 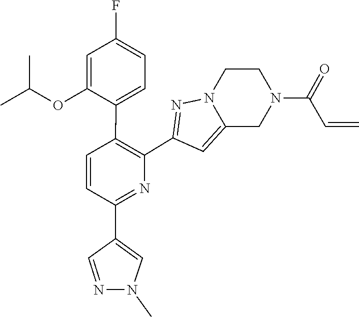 [SMILES: C=CC(=O)N1CCn2nc(-c3nc(-c4cnn(C)c4)ccc3-c3ccc(F)cc3OC(C)C)cc2C1] | LCMS m/z [M + 1]: 487.3 ¹H NMR (400 MHz, CD₃OD) δ 8.52 (s, 1H), 8.27 (s, 1H), 8.16 (d, J = 8.3 Hz 1H), 8.04 (d, J = 8.3 Hz, 1H), 7.28 (t, J = 8.0 Hz, 1H), 6.79-6.87 (m, 3H), 6.28 (d, J = 16.6 Hz 1H), 5.78-5.82 (m, 2H), 4.75-4.83 (m, 2H), 4.45-4.51 (m, 1H), 4.33 (s, 2H), 4.13-4.15 (m, 2H), 4.03 (s, 3H), 1.00 (m, 6H). |
| 280 | 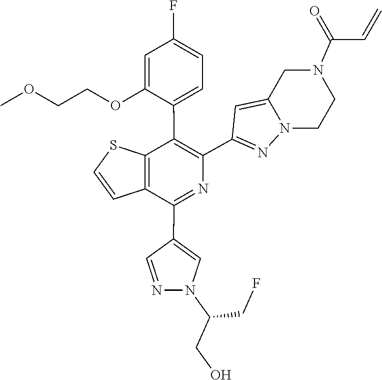 [SMILES: C=CC(=O)N1CCn2nc(-c3nc(-c4cnn([C@@H](CO)CF)c4)c4ccsc4c3-c3ccc(F)cc3OCCOC)cc2C1] | LCMS m/z [M + 1]: 621.3 ¹H NMR (400 MHz, CD₃OD) δ 8.76 (s, 1H), 8.41 (s, 1H), 8.06 (dd, J = 5.5, 17.8 Hz, 2H), 7.38 (t, J-7.4 Hz, 1H), 7.07 (d, J = 10.3 Hz, 1H), 6.71-6.95 (m, 2H), 6.28 (d, J = 16.9 Hz, 1H), 5.82-5.85 (m, 2H), 4.93- 5.05 (m, 2H), 4.75-4.82 (m, 3H), 3.97-4.16 (m,8H), 3.4 (t, J = 4.3 Hz, 2H), 3.06 (s, 3H). |
| 281 | 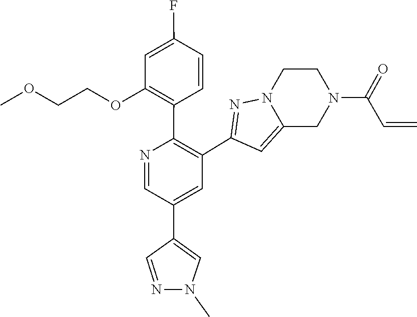 [SMILES: C=CC(=O)N1CCn2nc(-c3cc(-c4cnn(C)c4)cnc3-c3ccc(F)cc3OCCOC)cc2C1] | LCMS m/z [M + 1]: 503.3 ¹H NMR (400 MHz, CD₃OD) δ 8.96 (s, 2H), 8.35 (s, 1H), 8.13 (s, 1H), 7.41 (t, J = 8.1 Hz, 1H), 7.04 (dd, J = 10.9, 1.7 Hz, 1H), 6.72-6.94 (m, 2H), 6.28 (d, J = 16.4 Hz, 1H), 5.67-5.84 (m, 2H), 4.70-4.85(m, 2H), 4.25 (s, 2H), 4.12 (t, J = 5.50 Hz, 2H), 4.02 (t, J = 4.2 Hz, 2H), 4.00 (s, 3H), 3.46 (t, J = 4.5 Hz, 2H), 3.19 (s, 3H). |
| 282 | 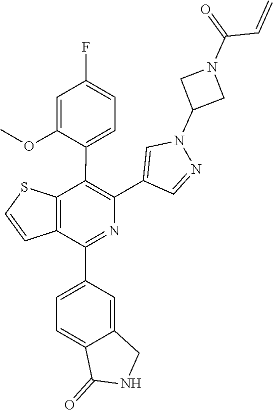 [SMILES: C=CC(=O)N1CC(n2cc(-c3nc(-c4ccc5c(c4)CNC5=O)c4ccsc4c3-c3ccc(F)cc3OC)cn2)C1] | LCMS m/z [M + 1]: 566.4 ¹H NMR (400 MHz, DMSO-d6) δ 8.68 (brs, 1H), 8.15 (s, 1H), 8.07 (d, J = 8.0 Hz, 1H), 7.91 (s, 1H), 7.87-7.84 (m, 2H), 7.71 (d, J = 5.6 Hz, 1H), 7.36 (t, J = 7.6 Hz, 1H), 7.22 (dd, J = 11.2, 2.4 Hz, 1H), 7.13 (s, 1H), 7.01 (td, J = 8.0, 2.4 Hz, 1H), 6.33 (dd, J = 16.8, 10.4 Hz, 1H), 6.12 (dd, J = 16.8, 2.0 Hz, 1H), 5.69 (dd, J = 10.4, 2.0 Hz, 1H), 5.32-5.26 (m, 1H), 4.63 (t, J = 8.0 Hz, 1H), 4.52 (s, 2H), 4.42-4.31 (m, 2H), 4.16-4.09 (m, 1H), 3.66 (s, 3H). |
| 283 | 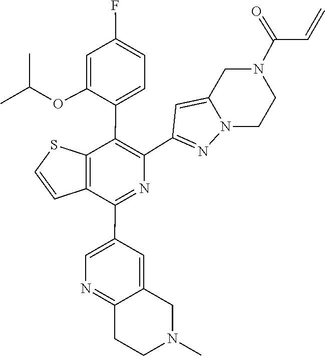 [SMILES: C=CC(=O)N1CCn2nc(-c3nc(-c4cnc5c(c4)CN(C)CC5)c4ccsc4c3-c3ccc(F)cc3OC(C)C)cc2C1] | LCMS m/z [M + 1]: 609.2 ¹H NMR (400 MHz, CD₃OD) 9.16 (s, 1H), 8.37 (s, 1H), 7.87(d, J = 5.6 Hz, 1H), 7.72 (d, J = 5.6 Hz, 1H), 7.34 (t, J = 7.2 Hz, 1H), 6.71-6.98 (m, 3H), 6.27 (d, J = 16.8 Hz, 1H), 5.75-5.86 (m, 2H), 4.60-4.85 (m, 4H), 4.46-4.56 (m, 1H), 4.24 (s, 2H), 4.10-4.19 (m, 2H), 3.70-3.98 (m, 2H), 3.40-3.48 (m, 2H), 3.17(s, 3H), 1.07 (d, J = 6.0 Hz, 3H), 0.90 (d, J = 6.0 Hz, 3H) |
| 284 | 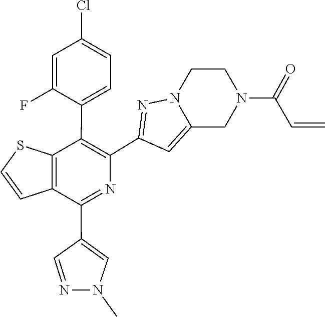 [SMILES: C=CC(=O)N1CCn2nc(-c3nc(-c4cnn(C)c4)c4ccsc4c3-c3ccc(Cl)cc3F)cc2C1] | LCMS m/z [M + 1]: 519.2 ¹H NMR (400 MHz, CD₃OD) 8.58 (s, 1H), 8.31 (s, 1H), 8.03 (s, 2H), 7.52 (t, J = 8.0 Hz, 1H), 7.43 (d, J = 8.8 Hz, 2H), 6.74-6.90 (m, 1H), 6.28 (d, J = 16.4 Hz, 1H), 6.03 (d, J = 9.2 Hz, 1H), 5.82 (d, J = 10.8 Hz, 1H), 4.83 (m, 2H), 4.25 (s, 2H), 4.13 (t, J = 5.2 Hz, 2H), 4.08 (s, 3H). |
| 285 | 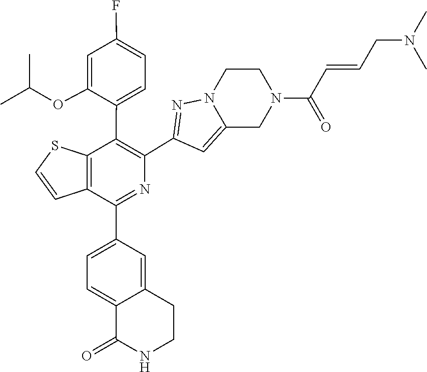 [SMILES: CC(C)Oc1cc(F)ccc1-c1c(-c2cc3n(n2)CCN(C(=O)/C=C/CN(C)C)C3)nc(-c2ccc3c(c2)CCNC3=O)c2ccsc12] | LCMS m/z [M + 1]: 665.3 ¹H NMR (400 MHz, CD₃OD) 8.22 (d, J = 7.9 Hz, 1H), 7.93-8.05 (m, 3H), 7.80 (d, J = 5.6 Hz, 1H), 7.35-7.43 (m, 1H), 6.84-7.04 (m, 3H), 6.70-6.81 (m, 1H), 5.84 (d, J = 9.7 Hz, 1H), 4.78 (s, 2H), 4.52-4.61 (m, 1H), 4.25-4.37 (m, 2H), 4.07-4.21 (m, 2H), 3.90- 3.42 (m, 2H), 3.62 (t, J = 6.6 Hz, 2H), 3.18 (t, J = 6.5 Hz, 2H), 2.84-2.97 (m, 6H), 1.10 (d, J = 6.0 Hz, 3H), 0.94 (d, J = 6.0 Hz, 3H). |
| 286 | 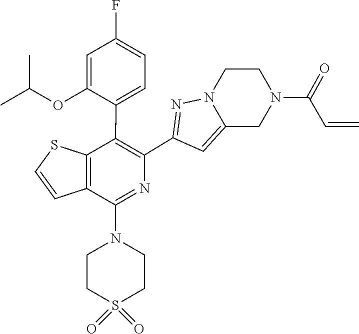 [SMILES: C=CC(=O)N1CCn2nc(-c3nc(N4CCS(=O)(=O)CC4)c4ccsc4c3-c3ccc(F)cc3OC(C)C)cc2C1] | LCMS m/z [M + 1]: 596.3 ¹H NMR (400 MHz, CD₃OD) 7.75-7.77 (m, 1H), 7.69 (d, J = 5.2 Hz, 1H), 7.25 (t, J = 8.0 Hz, 1H), 6.77- 6.92 (m, 3H), 6.27 (d, J = 16.4 Hz, 1H), 5.81 (d, J = 4.8 Hz, 1H), 5.75 (s, 1H), 4.82 (s, 1H), 4.73 (m, 1H), 4.45-4.52 (m, 1H), 4.21-4.29 (m, 6H), 4.11 (t, J = 5.6 Hz, 2H), 3.34-3.48 (m, 4H), 1.06 (d, J = 6.0 Hz, 3H), 0.90 (d, J = 6.0 Hz, 3H). |
| 287 | 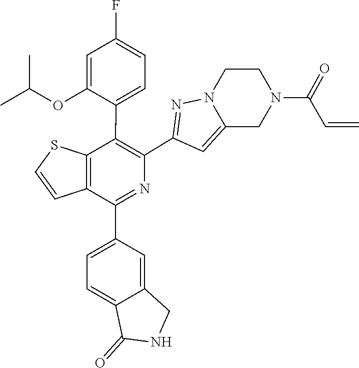 [SMILES: C=CC(=O)N1CCn2nc(-c3nc(-c4ccc5c(c4)CNC5=O)c4ccsc4c3-c3ccc(F)cc3OC(C)C)cc2C1] | LCMS m/z [M + 1]: 594.3 ¹H NMR (400 MHz, CD₃OD) 8.23 (s, 1H), 8.05-8.17 (m, 3H), 7.81 (d, J = 5.6 Hz, 1H), 7.42 (t, J = 7.5 Hz, 1H), 7.01 (d, J = 11.2 Hz, 1H), 6.79-6.96 (m, 2H), 6.28 (d, J = 16.8 Hz, 1H), 5.82 (s, 2H), 4.76 (s, 2H), 4.73-4.84 (s, 2H), 4.53-4.61 (m, 1H), 4.30 (s, 2H), 4.13 (t, J = 5.6 Hz, 2H), 1.10 (d, J = 6.0 Hz, 3H), 0.93 (d, J = 6.0 Hz, 3H). |
| 288 | 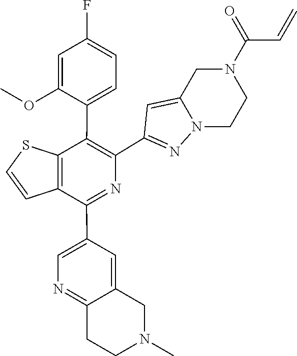 [SMILES: C=CC(=O)N1CCn2nc(-c3nc(-c4cnc5c(c4)CN(C)CC5)c4ccsc4c3-c3ccc(F)cc3OC)cc2C1] | LCMS m/z [M + 1]: 581.2 ¹H NMR (400 MHz, CD₃OD) 9.13 (s, 1H), 8.32 (s, 1H), 7.84 (d, J = 5.6 Hz, 1H), 7.70 (d, J = 5.6 Hz, 1H), 7.29 (t, J = 7.2 Hz, 1H), 6.77-6.98 (m, 3H), 6.27 (d, J = 16.8 Hz, 1H), 5.75-5.90 (m, 2H), 4.60-4.85 (m, 4H), 4.22 (s, 2H), 4.10-4.19 (m, 2H), 3.70-3.98 (m, 2H), 3.63 (s, 3H), 3.40-3.48 (m, 2H), 3.17(s, 3H) |
| 289 | 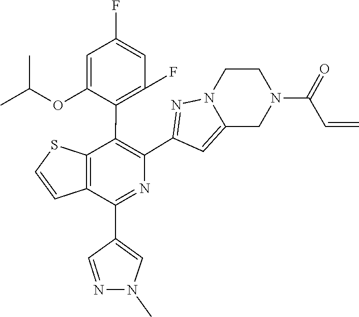 [SMILES: C=CC(=O)N1CCn2nc(-c3nc(-c4cnn(C)c4)c4ccsc4c3-c3c(F)cc(F)cc3OC(C)C)cc2C1] | LCMS m/z [M + 1]: 561.2 ¹H NMR (400 MHz, CD₃OD) 8.68 (s, 1H), 8.36 (s, 1H), 8.16 (d, J = 5.6 Hz, 1H), 8.09 (d, J = 5.6 Hz, 1H), 6.94 (d, J = 11.2 Hz, 1H), 6.74-6.90 (m, 2H), 6.29 (d, J = 16.8 Hz, 1H), 5.97 (s, 1H), 5.84 (d, J = 9.6 Hz, 1H), 4.74-4.87 (m, 2H), 4.58-4.66 (m, 1H), 4.28-4.39 (m, 2H), 4.16 (t, J = 5.4 Hz, 2H), 4.11 (s, 3H), 1.10 (d, J = 6.0 Hz, 3H), 0.97 (d, J = 5.6 Hz, 3H). |
| 291 | 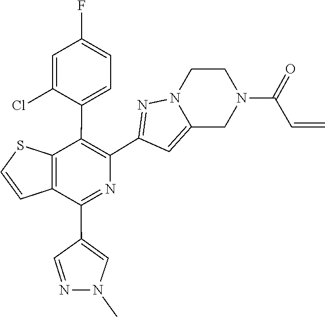 [SMILES: C=CC(=O)N1CCn2nc(-c3nc(-c4cnn(C)c4)c4ccsc4c3-c3ccc(F)cc3Cl)cc2C1] | LCMS m/z [M + 1]: 519.1 ¹H NMR (400 MHz, CD₃OD) 8.65 (s, 1H), 8.35 (s, 1H), 8.07-8.10 (m, 2H), 7.53-7.59 (m, 2H), 7.33-7.38 (m, 1H), 6.76-6.91 (m, 1H), 6.28 (d, J = 16.8 Hz, 1H), 5.83-5.85 (m, 2H), 4.78-4.85 (m, 2H), 4.29-4.31 (m, 2H), 4.11-4.16 (m, 2H), 4.10 (s, 3H). |
| 292 | 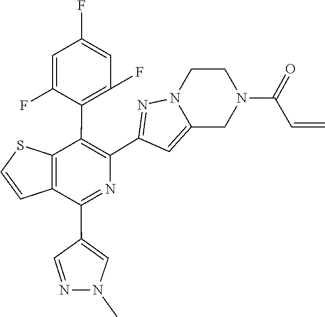 [SMILES: C=CC(=O)N1CCn2nc(-c3nc(-c4cnn(C)c4)c4ccsc4c3-c3c(F)cc(F)cc3F)cc2C1] | LCMS m/z [M + 1]: 521.2 ¹H NMR (400 MHz, CD₃OD) 8.55 (s, 1H), 8.31 (s, 1H), 7.95-8.02 (m, 2H), 7.08 (t, J = 8.4 Hz, 2H), 6.73- 6.93 (m, 1H), 6.24-6.36 (m, 2H), 5.82 (d, J = 10.8 Hz, 1H), 4.09-4.23 (m, 4H), 4.07 (s, 3H). |
| 293 | 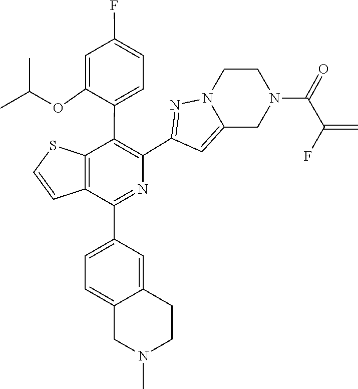 [SMILES: C=C(F)C(=O)N1CCn2nc(-c3nc(-c4ccc5c(c4)CCN(C)C5)c4ccsc4c3-c3ccc(F)cc3OC(C)C)cc2C1] | LCMS m/z [M + 1]: 626.4 ¹H NMR (400 MHz, CD₃OD) 8.04 (d, J = 5.6 Hz, 1H), 7.91-7.93 (m, 2H), 7.75 (d, J = 5.6 Hz, 1H), 7.53 (d, J = 8.0 Hz, 1H), 7.38 (t, J = 8.0 Hz, 1H), 6.97 (d, J = 11.2 Hz, 1H), 6.87 (t, J = 6.4 Hz, 1H), 5.83 (s, 1H), 5.28-5.39 (m, 2H), 4.76-4.87 (m, 2H), 4.55 (t, J = 6.0 Hz, 2H), 4.31 (s, 2H), 4.10 (d, J = 5.6 Hz, 2H), 3.52-3.87 (m, 2H), 3.39 (s, 2H), 3.13 (s, 3H), 1.09 (d, J = 6.0 Hz, 3H), 0.92 (d, J = 6.0 Hz, 3H). |
| 294 | 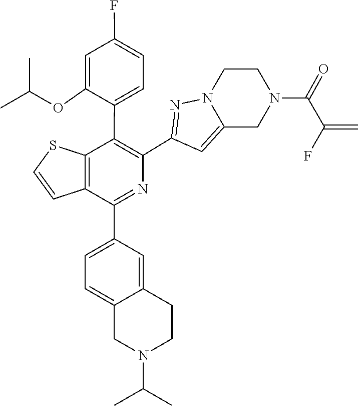 [SMILES: C=C(F)C(=O)N1CCn2nc(-c3nc(-c4ccc5c(c4)CCN(C(C)C)C5)c4ccsc4c3-c3ccc(F)cc3OC(C)C)cc2C1] | LCMS m/z [M + 1]: 654.4 ¹H NMR (400 MHz, CD₃OD) 8.05 (d, J = 5.6 Hz, 1H), 7.92-7.94 (m, 2H), 7.76 (d, J = 5.6 Hz, 1H), 7.59 (d, J = 7.6 Hz, 1H), 7.39 (t, J = 7.6 Hz, 1H), 6.99 (d, J = 10.8 Hz, 1H), 6.88 (t, J = 8.0 Hz, 1H), 5.83 (s, 1H), 5.28-5.39 (m, 2H), 4.76 (s, 2H), 4.64 (s, 2H), 4.55-4.59 (m, 1H), 4.32 (s, 2H), 4.10 (s, 2H), 3.78- 3.85 (m, 2H), 3.38-3.48 (m, 3H), 1.51 (d, J = 6.4 Hz, 6H), 1.09 (d, J = 5.6 Hz, 3H), 0.92 (d, J = 5.6 Hz, 3H). |
| 295 | 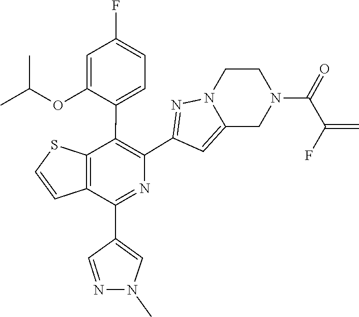 [SMILES: C=C(F)C(=O)N1CCn2nc(-c3nc(-c4cnn(C)c4)c4ccsc4c3-c3ccc(F)cc3OC(C)C)cc2C1] | LCMS m/z [M + 1]: 561.3 ¹H NMR (400 MHz, CD₃OD) 8.68 (s, 1H), 8.35 (s, 1H), 8.16 (d, J = 5.6 Hz, 1H), 8.08 (d, J = 5.6 Hz, 1H), 7.41 (t, J = 8.0 Hz, 1H), 7.03 (d, J = 11.2 Hz, 1H), 6.90 (t, J = 8.4 Hz, 1H), 5.78 (s, 1H), 5.29-5.40 (m, 2H), 4.77 (s, 2H), 4.54-4.60 (m, 1H), 4.38 (s, 2H), 4.11-4.13 (m, 5H), 1.08 (d, J = 5.6 Hz, 3H), 0.92 (d, J = 5.6 Hz, 3H). |
| 296 | 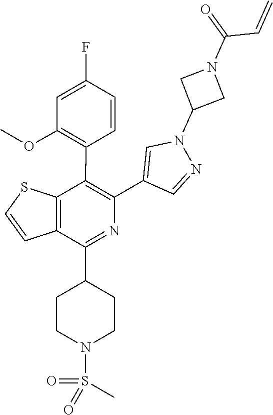 [SMILES: C=CC(=O)N1CC(n2cc(-c3nc(C4CCN(S(C)(=O)=O)CC4)c4ccsc4c3-c3ccc(F)cc3OC)cn2)C1] | LCMS m/z [M + 1]: 596.4 ¹H NMR (400 MHz, DMSO-d6) δ 7.93 (s, 1H), 7.74 (s, 2H), 7.29-7.25 (m, 1H), 7.18 (dd, J = 11.2, 2.4 Hz, 1H), 6.99-6.94 (m, 2H), 6.33 (dd, J = 16.8, 10.0 Hz, 1H), 6.12 (dd, J = 16.8, 2.4 Hz, 1H), 5.70-5.66 (m, 1H), 5.35-5.28 (m, 1H), 4.62 (t, J = 8.8 Hz, 1H), 4.41- 4.38 (m, 1H), 4.32 (t, J = 9.6 Hz, 1H), 4.13-4.10 (m, 1H), 3.78-3.74 (m, 2H), 3.62 (s, 3H), 3.53-3.45 (m, 1H), 3.01-2.94 (m, 5H), 2.17-1.98 (m, 4H) |
| 297 | 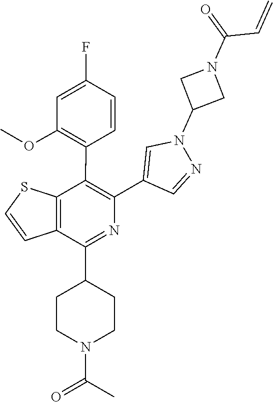 [SMILES: C=CC(=O)N1CC(n2cc(-c3nc(C4CCN(C(C)=O)CC4)c4ccsc4c3-c3ccc(F)cc3OC)cn2)C1] | LCMS m/z [M + 1]: 560.3 ¹H NMR (400 MHz, DMSO-d6) δ 7.85 (brs, 1H), 7.78 (d, J = 5.6 Hz, 1H), 7.73 (d, J = 5.6 Hz, 1H), 7.26 (t, J = 8.0 Hz, 1H), 7.17 (dd, J = 11.2, 2.0 Hz 1H), 7.01-6.94 (m, 2H), 6.33 (dd, J = 16.8, 10.0 Hz 1H), 6.12 (dd, J = 17.2, 2.4 Hz, 1H), 5.69 (dd, J = 10.4, 2.0 Hz, 1H), 5.33-5.27 (m, 1H), 4.64-4.59 (m, 2H), 4.40- 4.30 (m, 2H), 4.12-4.09 (m, 1H), 3.99 (t, J = 12.4 Hz, 1H), 3.62-3.57 (m, 4H), 3.31-3.28 (m, 1H), 2.78 (td, J = 12.4, 2.8 Hz, 1H), 2.07 (s, 3H), 2.01-1.79 (m, 4H) |
| 298 | 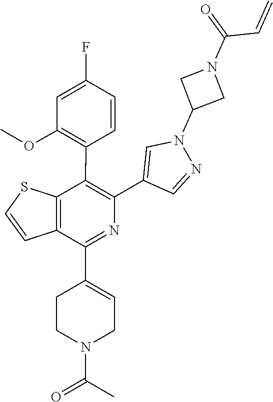 [SMILES: C=CC(=O)N1CC(n2cc(-c3nc(C4=CCN(C(C)=O)CC4)c4ccsc4c3-c3ccc(F)cc3OC)cn2)C1] | LCMS m/z [M + 1]: 558.4 ¹H NMR (400 MHz, DMSO-d6) δ 7.85 (d, J = 20.8 Hz, 1H), 7.75-7.71 (m, 2H), 7.29 (t, J = 7.6 Hz, 1H), 7.18 (dd, J = 11.6, 2.4 Hz, 1H), 7.06 (d, J = 20.8 Hz, 1H), 6.98 (td, J = 8.4, 2.4 Hz, 1H), 6.43 (d, J = 16.0 Hz, 1H), 6.33 (dd, J = 17.2, 10.4 Hz, 1H), 6.12 (dd, J = 16.8, 2.0 Hz, 1H), 5.69 (dd, J = 10.4, 2.0 Hz, 1H), 5.29-5.24 (m, 1H), 4.62 (t, J = 9.2 Hz, 1H), 4.41-4.24 (m, 4H), 4.13-4.12 (m, 1H), 3.77-3.71 (m, 2H), 3.62 (s, 3H), 2.84 (m, 2H), 2.11 (d, J = 8.8 Hz, 3H). |
| 299 | 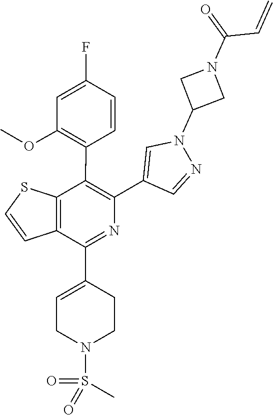 [SMILES: C=CC(=O)N1CC(n2cc(-c3nc(C4=CCN(S(C)(=O)=O)CC4)c4ccsc4c3-c3ccc(F)cc3OC)cn2)C1] | LCMS m/z [M + 1]: 594.4 ¹H NMR (400 MHz, DMSO-d6) δ 7.86 (s, 1H), 7.78- 7.75 (m, 2H), 7.31-7.27 (m, 1H), 7.19 (dd, J = 11.2, 2.4 Hz, 1H), 7.06 (s, 1H), 6.98 (td, J = 8.4, 2.4 Hz, 1H), 6.46 (t, J = 1.6 Hz, 1H), 6.33 (dd, J = 16.8, 10.0 Hz, 1H), 6.12 (dd, J = 17.2, 2.4 Hz, 1H), 5.69 (dd, J = 10.4, 2.4 Hz, 1H), 5.30-5.24 (m, 1H), 4.63 (t, J = 8.8 Hz, 1H), 4.42-4.31 (m, 2H), 4.13-4.11 (m, 1H), 4.02 (d, J = 3.2 Hz, 2H), 3.63 (s, 3H), 3.49 (t, J = 6.0 Hz, 2H), 3.01 (s, 3H), 2.96 (brs, 2H) |
| 300 | 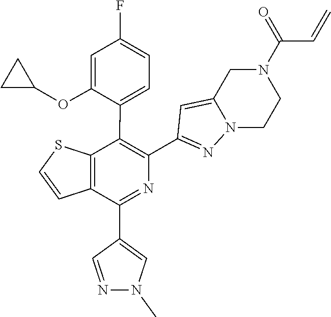 | LCMS m/z [M + 1]: 606.2 ¹H NMR (400 MHz, CD₃OD) 7.97 (d, J = 5.6 Hz, 1H), 7.89-7.95 (m, 2H), 7.73 (d, J = 5.6 Hz, 1H), 7.51 (d, J = 8.0 Hz, 1H), 7.39-7.45 (m, 1H), 7.21-7.28 (m, 1H), 6.80-6.97 (m, 2H), 6.29 (d, J = 16.8 Hz, 1H), 5.75-5.87 (m, 2H), 4.66-4.85 (m, 3H), 4.45-4.59 (m, 1H), 4.24-4.33 (m, 2H), 4.12-4.17 (m, 2H), 3.80-3.94 (m, 1H), 3.68-3.76 (m, 1H), 3.35-3.51 (m, 3H), 3.13 (s, 3H), 0.61-0.72 (m, 2H), 0.47-0.54 (m, 1H), 0.00- 0.05 (m, 1H). |
| 301 | 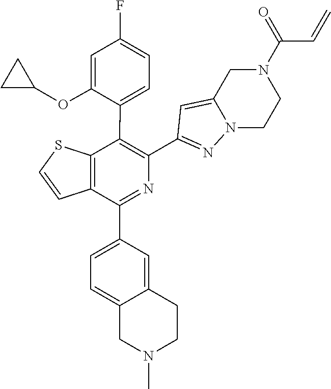 | LCMS m/z [M + 1]: 541.2 ¹H NMR (400 MHz, CD₃OD) 8.43 (s, 1H), 8.27 (s, 1H), 7.87 (d, J = 5.6 Hz, 1H), 7.74 (d, J = 5.6 Hz, 1H), 7.31-7.36 (m, 1H), 7.15 (d, J = 10.8 Hz, 1H), 6.75-6.94 (m, 2H), 6.28 (d, J = 17.2 Hz, 1H), 5.83 (d, J = 10.8 Hz, 1H), 5.76 (s, 1H), 4.67-4.79 (m, 2H), 4.18-4.27 (m, 2H), 4.10-4.16 (m, 2H), 4.03 (s, 3H), 3.63-3.68 (m, 1H), 0.56-0.66 (m, 2H), 0.47-0.54 (m, 1H), 0.08-0.01 (m, 1H). |

In some embodiments, the present disclosure provides compounds selected from those included in Table 2, or pharmaceutically acceptable salts thereof.

Lengthy table referenced here
US20250230138A1-20250717-T00001
Please refer to the end of the specification for access instructions.

In some embodiments, provided compounds are provided and/or utilized in a salt form (e.g., a pharmaceutically acceptable salt form). Reference to a compound provided herein is understood to include reference to salts thereof, unless otherwise indicated. Pharmaceutically acceptable salt forms are known in the art. For example, S. M. Berge, et al. describes pharmaceutically acceptable salts in detail in J. Pharmaceutical Sciences, 66:1-19(1977). Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

It will be appreciated that compounds described herein may be provided and/or utilized in any available form (e.g., a salt form) and that all such forms are contemplated by the present disclosure. The present disclosure also contemplates forms such as esters, tautomers, prodrugs, zwitterionic forms, and stereoisomers of the compounds provided herein.

It will be appreciated that throughout the present disclosure, unless otherwise indicated, reference to a compound of formula I is intended to also include formulae I-a, I-b, I-c, L-d, I-e, I-f, I-g, I-h, I-i, I-j, I-k, I-a-i, I-b-i, I-c-i, I-d-i, I-e-i, I-f-i, I-g-i, I-h-i, I-i-i, I-j-I, I-k-i, I-a-ii, I-b-ii, I-c-ii, I-d-ii, I-e-ii, I-f-ii, I-g-ii, I-h-ii, I-i-ii, I-j-ii, I-k-ii, I-a-ii′, I-b-ii′, I-c-ii′, I-d-ii′, I-e-ii′, I-f-ii′, I-g-ii′, I-h-ii′, I-i-ii′, I-j-ii′, I-k-ii, I-a-iii, I-b-iii, I-c-iii, I-d-iii, I-e-iii, I-f-iii, I-g-iii, I-h-iii, I-i-iii, I-j-iii, I-k-iii, I-a-iv, I-b-iv, I-c-iv, I-d-iv, I-e-iv, I-f-iv, I-g-iv, I-h-iv, I-i-iv, I-j-iv, I-k-iv, I-a-v, I-b-v, I-c-v, I-d-v, I-e-v, I-f-v, I-g-v, I-h-v, I-i-v, I-j-v, I-k-v, I-a-v′, I-b-v′, I-c-v′, I-d-v′, I-e-v′, I-f-v′, I-g-v′, I-h-v′, I-i-v′, I-j-v′, I-k-v′, I-l, I-m, I-n, I-p, I-q, I-r, IA, IA1, IB, IB1, IC, IC1, ID, ID1, IE, IE1, IF, and IF1, and compound species of such formulae disclosed herein.

In some embodiments, provided compounds are prepared as described herein, such as according to any one of Synthetic Examples 1-88.

### Compositions

The present disclosure also provides compositions comprising a compound provided herein with one or more other components. In some embodiments, provided compositions comprise and/or deliver a compound described herein (e.g., compounds of Formulae I, I-a, I-b, I-c, I-d, I-e, I-f, I-g, I-h, I-i, I-j, I-k, I-a-i, I-b-i, I-c-i, I-d-i, I-e-i, I-f-i, I-g-i, I-h-i, I-i-i, I-j-I, I-k-i, I-a-ii, I-b-ii, I-c-ii, I-d-ii, I-e-ii, I-f-ii, I-g-ii, I-h-ii, I-i-ii, I-j-ii, I-k-ii, I-a-ii′, I-b-ii′, I-c-ii′, I-d-ii′, I-e-ii′, I-f-ii′, I-g-ii′, I-h-ii′, I-i-ii′, I-j-ii′, I-k-ii′, I-a-iii, I-b-iii, I-c-iii, I-d-iii, I-e-iii, I-f-iii, I-g-iii, I-h-iii, I-i-iii, I-j-iii, I-k-iii, I-a-iv, I-b-iv, I-c-iv, I-d-iv, I-e-iv, I-f-iv, I-g-iv, I-h-iv, I-i-lv, I-j-iv, I-k-iv, I-a-v, I-b-v, I-c-v, I-d-v, I-e-v, I-f-v, I-g-v, I-h-v, I-i-v, I-j-v, I-k-v, I-a-v′, I-b-v′, I-c-v′, I-d-v′, I-e-v′, I-f-v′, I-g-v′, I-h-v′, I-i-v′, I-j-v′, I-k-v′, I-l, I-m, I-n, I-p, I-q, I-r, IA, IA1, IB, IB1, IC, IC1, ID, ID1, IE, IE1, IF, or IF1, or compounds selected from Table 1 or Table 2).

In some embodiments, a provided composition is a pharmaceutical composition that comprises and/or delivers a compound provided herein (e.g., compounds of Formulae I, I-a, I-b, I-c, I-d, I-e, I-f, I-g, I-h, I-i, I-j, I-k, I-a-i, I-b-i, I-c-j, I-d-i, I-e-i, I-f-i, I-g-i, I-h-i, I-i-i, I-j-i, I-k-i, I-a-ii, I-b-ii, I-c-ii, I-d-ii, I-e-ii, I-f-ii, I-g-ii, I-h-ii, I-i-ii, I-j-ii, I-k-ii, I-a-ii′, I-b-ii′, I-c-ii′, I-d-ii′, I-e-ii′, I-f-ii′, I-g-ii′, I-h-ii′, I-i-ii′, I-j-ii′, I-k-ii′, I-a-iii, I-b-iii, I-c-iii, I-d-iii, I-e-iii, I-f-iii, I-g-iii, I-h-iii, I-i-iii, I-j-ii, I-k-iii, I-a-iv, I-b-iv, I-c-iv, I-d-iv, I-e-iv, I-f-iv, I-g-iv, I-h-iv, I-i-iv, I-j-iv, I-k-iv, I-a-v, I-b-v, I-c-v, I-d-v, I-e-v, I-f-v, I-g-v, I-h-v, I-i-v, I-j-v, I-k-v, I-a-v′, I-b-v′, I-c-v′, I-d-v′, I-e-v′, I-f-v′, I-g-v′, I-h-v′, I-i-v′, I-j-v′, I-k-v′, I-l, I-m, I-n, I-p, I-q, I-r, IA, IA1, IB, IB1, IC, IC1, ID, ID1, IE, IE1, IF, or IF1, or compounds selected from Table 1 or Table 2) and further comprises a pharmaceutically acceptable carrier. Pharmaceutical compositions typically contain an active agent (e.g., a compound described herein) in an amount effective to achieve a desired therapeutic effect while avoiding or minimizing adverse side effects. In some embodiments, provided pharmaceutical compositions comprise a compound described herein and one or more fillers, disintegrants, lubricants, glidants, anti-adherents, and/or anti-statics, etc. Provided pharmaceutical compositions can be in a variety of forms including oral dosage forms, topical creams, topical patches, iontophoresis forms, suppository, nasal spray and/or inhaler, eye drops, intraocular injection forms, depot forms, as well as injectable and infusible solutions. Methods of preparing pharmaceutical compositions are well known in the art.

In some embodiments, provided compounds are formulated in a unit dosage form for ease of administration and uniformity of dosage. The expression “unit dosage form” as used herein refers to a physically discrete unit of an active agent (e.g., a compound described herein) for administration to a subject. Typically, each such unit contains a predetermined quantity of active agent. In some embodiments, a unit dosage form contains an entire single dose of the agent. In some embodiments, more than one unit dosage form is administered to achieve a total single dose. In some embodiments, administration of multiple unit dosage forms is required, or expected to be required, in order to achieve an intended effect. A unit dosage form may be, for example, a liquid pharmaceutical composition containing a predetermined quantity of one or more active agents, a solid pharmaceutical composition (e.g., a tablet, a capsule, or the like) containing a predetermined amount of one or more active agents, a sustained release formulation containing a predetermined quantity of one or more active agents, or a drug delivery device containing a predetermined amount of one or more active agents, etc.

Provided compositions may be administered using any amount and any route of administration effective for treating or lessening the severity of any disease or disorder described herein.

### Uses

The present disclosure provides uses for compounds and compositions described herein (e.g., compounds of Formulae I, I-a, I-b, I-c, I-d, I-e, I-f, I-g, I-h, I-i, I-j, I-k, I-a-i, I-b-i, I-c-i, I-d-i, I-e-i, I-f-i, I-g-i, I-h-i, I-i-i, I-j-I, I-k-i, I-a-ii, I-b-ii, I-c-ii, I-d-ii, I-e-ii, I-f-ii, I-g-ii, I-h-ii, I-i-ii, I-j-ii, I-k-ii, I-a-ii′, I-b-ii′, I-c-ii′, I-d-ii′, I-e-ii′, I-f-ii′, I-g-ii′, I-h-ii′, I-i-ii′, I-j-ii, I-k-ii′, I-a-iii, I-b-iii, I-c-iii, I-d-iii, I-e-iii, I-f-iii, I-g-iii, I-h-iii, I-i-iii, I-j-iii, I-k-iii, I-a-iv, I-b-iv, I-c-iv, I-d-iv, I-e-iv, I-f-iv, I-g-iv, I-h-iv, I-i-iv, I-j-iv, I-k-iv, I-a-v, I-b-v, I-c-v, I-d-v, I-e-v, I-f-v, I-g-v, I-h-v, I-i-v, I-j-v, I-k-v, I-a-v′, I-b-v′, I-c-v′, I-d-v′, I-e-v′, I-f-v′, I-g-v′, I-h-v′, I-i-v′, I-j-v′, I-k-v′, I-l, I-m, I-n, I-p, I-q, I-r, IA, IA1, IB, IB1, IC, IC1, ID, ID1, IE, IE1, IF, or IF1, or compounds selected from Table 1 or Table 2). In some embodiments, provided compounds and compositions are useful in medicine (e.g., as therapeutic agents for use in the treatment, amelioration, delaying progress of, amelioration or elimination of a symptom of, and/or inhibition of a disease or disorder, as described herein). In some embodiments, provided compounds and compositions are useful as medicaments. In some embodiments, provided compounds and compositions are useful in research as, for example, analytical tools and/or control compounds in biological assays.

In some embodiments, provided compounds are useful for disrupting (e.g., inhibiting and/or preventing and/or modulating) an interaction between a small GTPase (e.g., Rac1, CDC42, or a RAS protein such as KRAS, NRAS, HRAS, RRAS, RRAS2, MRAS, or RIT1) and a PI3Kα protein. In some embodiments, the present disclosure provides methods of disrupting, interrupting, and/or preventing an interaction between a small GTPase (e.g., Rac1, CDC42, or a RAS protein such as KRAS, NRAS, HRAS, RRAS, RRAS2, MRAS, or RIT1) and a PI3Kα protein in a subject, comprising administering a provided compound or composition. In some embodiments, the present disclosure provides methods of disrupting, interrupting, and/or preventing an interaction between a small GTPase (e.g., Rac1, CDC42, or a RAS protein such as KRAS, NRAS, HRAS, RRAS, RRAS2, MRAS, or RIT1) and a PI3Kα protein in a biological sample, comprising administering a provided compound or composition. In some embodiments, the present disclosure provides methods comprising contacting a cell containing a small GTPase (e.g., Rac1, CDC42, or a RAS protein such as KRAS, NRAS, HRAS, RRAS, RRAS2, MRAS, or RIT1) and a PI3Kα protein with a provided compound or composition. In some such embodiments, a small GTPase is capable of binding a RAS-binding domain (RBD) of a PI3Kα protein. In some embodiments, a small GTPase is selected from Rac1, CDC42, and RAS proteins. In some embodiments, a RAS protein is selected from HRAS, NRAS, KRAS, RRAS, RRAS2, MRAS, and RIT1. In some embodiments, a RAS protein is a wild-type RAS protein. In some embodiments, a RAS protein is a mutant RAS protein. In some embodiments, a RAS protein (e.g., HRAS, NRAS, or KRAS) comprises a mutation in codon 12 (e.g., G12), codon 13 (e.g., G13), or codon 61 (e.g., Q61). In some embodiments, a KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, a KRAS protein comprises a G12C, G12D, G12S, G12V, G12R, G13D, and/or Q61H mutation. In some embodiments, an NRAS protein comprises a G12C, G12D, G12S, G12V. G12R, G12A, G3D, G13A, G13C, G13R, G13S, G3V, Q61E, Q61K, Q61L, Q61P, Q61R, and/or Q61H mutation. In some embodiments, an NRAS protein comprises a G12D, G12V, G13D, and/or Q61R mutation. In some embodiments, an HRAS protein comprises a G12C, G12D, G12S, G12V, G12R, G12A, G13D, G13C, G13R, G13S, G13V, Q61K, Q61L, Q61P. Q61R, and/or Q61H mutation. In some embodiments, an HRAS protein comprises a G12V, G13R, and/or Q61R mutation. In some embodiments, a PI3Kα protein is a wild-type PI3Kα protein. In some embodiments, a PI3Kα protein is a mutant PI3Kα protein. In some embodiments, a PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation. In some embodiments, a PI3Kα protein comprises a E542K, E545K, H1047R, and/or H1047L mutation.

In some embodiments, the present disclosure provides methods comprising administering a provided compound or composition to a subject in need thereof. In some such embodiments, a subject has a disease, disorder, or condition associated with an interaction between a small GTPase (e.g., Rac1, CDC42, or a RAS protein such as KRAS, NRAS, HRAS, RRAS, RRAS2, MRAS, or RIT1) and a PI3Kα protein. In some embodiments, a subject has a disease, disorder, or condition ameliorated by disruption of an interaction between a small GTPase (e.g., Rac1, CDC42, or a RAS protein such as KRAS, NRAS, HRAS, RRAS, RRAS2, MRAS, or RIT1) and a PI3Kα protein. In some embodiments, a small GTPase (e.g., Rac1, CDC42, or a RAS protein such as KRAS, NRAS, HRAS, RRAS, RRAS2, MRAS, or RIT1) is capable of binding a RAS-binding domain (RBD) of a PI3Kα protein. In some embodiments, a small GTPase is selected from Rac1, CDC42, and RAS proteins. In some embodiments, a RAS protein is selected from HRAS, NRAS, KRAS, RRAS, RRAS2, MRAS, and RIT1. In some embodiments, a KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, a KRAS protein comprises a G12C, G12D, G12S, G12V, G12R, G13D, and/or Q61H mutation. In some embodiments, an NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, an NRAS protein comprises a G12D, G12V, G13D, and/or Q61R mutation. In some embodiments, an HRAS protein comprises a G12C, G12D, G12S, G12V, G12R, G12A, G13D, G13C, G13R, G13S, G13V, Q61K, Q61L, Q61P, Q61R, and/or Q61H mutation. In some embodiments, an HRAS protein comprises a G12V, G13R, and/or Q61R mutation. In some embodiments, a RAS protein is a wild-type RAS protein. In some embodiments, a PI3Kα protein is a wild-type PI3Kα protein. In some embodiments, a PI3Kα protein is a mutant PI3Kα protein. In some embodiments, a PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation. In some embodiments, a PI3Kα protein comprises a E542K, E545K, H1047R, and/or H1047L mutation. In some embodiments, a subject has a cancer or other indication described herein. In some embodiments, a subject has previously undergone a treatment regimen for a cancer. In some embodiments, a subject has previously entered remission from a cancer.

In some embodiments, provided methods comprise administering a provided compound or composition to a subject in need thereof, according to a regimen such that the subject does not experience hyperglycemia or insulin-driven resistance.

In some embodiments, the present disclosure provides methods of treating a cancer, comprising administering to a subject a provided compound or composition. In some embodiments, a cancer is associated with and/or characterized by aberrant activation of PI3Kα. In some embodiments, a cancer is characterized by a mutation in a RAS protein (e.g., HRAS, NRAS, KRAS, RRAS, RRAS2, MRAS, and RIT1). In some embodiments, a cancer is characterized by a mutation in a KRAS protein. In some embodiments, a KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, a KRAS protein comprises a G12C, G12D, G12S, G12V, G12R, G13D, and/or Q61H mutation. In some embodiments, a cancer is characterized by a mutation in an NRAS protein. In some embodiments, an NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, an NRAS protein comprises a G12D, G12V, G13D, and/or Q61R mutation. In some embodiments, a cancer is characterized by a mutation in an HRAS protein. In some embodiments, an HRAS protein comprises a G12C, G12D, G12S, G12V, G12R, G12A, G13D, G13C, G13R, G13S, G13V, Q61K, Q61L, Q61P, Q61R, and/or Q61H mutation. In some embodiments, an HRAS protein comprises a G12V, G13R, and/or Q61R mutation. In some embodiments, a cancer is characterized by a mutation in a PI3Kα protein. In some embodiments, a PI3Kα protein comprises a N345K, E726K, C420R, Q546R, G118D, E453K, Q546K, G1049R, M1043I, K111E, K111N, E81K, E545A, E545G, N1044K, E110del, Q546P, E542K, E545K, H1047R, and/or H1047L mutation. In some embodiments, a PI3Kα protein comprises a E542K, E545K, H1047R, and/or H1047L mutation.

As used herein, “cancer” (and also, “malignancy”, “neoplasm”, “tumor”, and “carcinoma”), refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. In some embodiments, a cancer may be characterized by a solid tumor. In some embodiments, a cancer may be characterized by a hematologic tumor. Numerous different types of cancers are known.

In some embodiments, a cancer is selected from pancreatic cancer; colon cancer; rectal cancer; colorectal cancer; breast cancer; ovarian cancer; endometrial cancer; lung cancer; prostate cancer; cancers of the oral cavity and pharynx (lip, tongue, mouth, larynx, pharynx), esophagus, stomach, small intestine, large intestine, liver and biliary passages, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease; non-Hodgkin's lymphomas; multiple myeloma; and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML), and lymphomas including lymphocytic, granulocytic and monocytic lymphomas. Additional exemplary types of cancer include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor. In some embodiments, a cancer is selected from breast cancer, lung cancer (e.g., non-small cell lung cancer), endometrial cancer, esophageal cancer, ovarian cancer, colorectal cancer, gastric cancer, squamous cell carcinoma, prostate cancer, and pancreatic cancer.

In some embodiments, a cancer is characterized by one or more mutations. In some such embodiments, a subject may be diagnosed with cancer and/or selected for therapy based on the detection of one or more mutations in a biological sample obtained from the subject. In some embodiments, a cancer is characterized by a mutation in a RAS protein (e.g., KRAS, HRAS, or NRAS). In some embodiments, a cancer is characterized by a mutation in a KRAS protein. In some embodiments, a KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, a KRAS protein comprises a G12C, G12D, G12S, G12V, G12R, G13D, and/or Q61H mutation. In some embodiments, a cancer is characterized by a mutation in an NRAS protein. In some embodiments, an NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, an NRAS protein comprises a G12D, G12V, G13D, and/or Q61R mutation. In some embodiments, a cancer is characterized by a mutation in an HRAS protein. In some embodiments, an HRAS protein comprises a G12C, G12D, G12S, G12V, G12R, G12A, G13D, G13C, G13R, G13S, G13V, Q61K, Q61L, Q61P, Q61R, and/or Q61H mutation. In some embodiments, an HRAS protein comprises a G12V, G13R, and/or Q61R mutation. In some embodiments, a cancer is characterized by a mutation in a PI3Kα protein. In some embodiments, the PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation. In some embodiments, a PI3Kα protein comprises a E542K, E545K, H1047R, and/or H1047L mutation. In some embodiments, a cancer is characterized by mutated, overexpressed, and/or amplified receptor tyrosine kinases (e.g., HER family (e.g., HER2 and/or HER3), Met, FGFR, Alk, PDGF, EGFR, or ROS kinases). In some embodiments, a cancer is characterized by a mutation in or a deletion of a PTEN protein. In some embodiments, a cancer has demonstrable sensitivity to Avastin. For example, a cancer may be non-small cell lung cancer (NSCLC) or colorectal cancer. In some embodiments, a cancer is ER positive (e.g., having estrogen receptors). In some embodiments, a cancer is PR positive (e.g., having progesterone receptors).

In some embodiments, the present disclosure provides methods of treating a metabolic syndrome, comprising administering to a subject a provided compound or composition. In some embodiments, a metabolic syndrome is selected from hyperinsulinemia and type 2 diabetes.

In some embodiments, the present disclosure provides methods of treating a RASopathy (e.g., a genetic syndrome caused by a germline mutation in a gene that encodes a component or regulator of the RAS/MAPK pathway), comprising administering to a subject a provided compound or composition. In some embodiments, a RASopathy is selected from the group consisting of capillary malformation-arteriovenous malformation syndrome and Legius syndrome. In some embodiments, a RASopathy is neurofibromatosis type 1 (NF1).

In some embodiments, the present disclosure provides methods of treating a vascular disorder, comprising administering to a subject a provided compound or composition. In some embodiments, a vascular disorder is selected from PIK3CA-related overgrowth syndrome (PROS) and vascular malformations (e.g., venous malformations; lymphatic malformations; congenital lipomatous overgrowth with vascular, epidermal, and skeletal anomalies syndrome (CLOVES); Klippel-Trenaunay Syndrome; PTEN hamartoma tumor syndrome (PHTS); and fibro-adipose vascular anomaly (FAVA)).

In some embodiments, the present disclosure provides methods of treating pulmonary hypertension, such as pulmonary arterial hypertension, comprising administering to a subject a provided compound or composition.

In some embodiments, the present disclosure provides methods of treating age-related macular degeneration or diabetic macular edema, comprising administering to a subject a provided compound or composition

In some embodiments, the present disclosure provides compounds or compositions for use in the manufacture of a medicament. In some embodiments, provided compounds or compositions are useful in the manufacture of a medicament for treating a disease, disorder, or condition associated with or ameliorated by an interaction between a small GTPase (e.g., Rac1, CDC42, or a RAS protein such as KRAS, NRAS, HRAS, RRAS, RRAS2, MRAS, or RIT1) and a PI3Kα protein. In some embodiments, a small GTPase (e.g., Rac1, CDC42, or a RAS protein such as KRAS, NRAS, HRAS, RRAS, RRAS2, MRAS, or RIT1) is capable of binding a RAS-binding domain (RBD) of a PI3Kα protein. In some embodiments, a small GTPase is selected from Rac1, CDC42, and RAS proteins. In some embodiments, a RAS protein is selected from HRAS, NRAS, KRAS, RRAS, RRAS2, MRAS, and RIT1. In some embodiments, a RAS protein is a wild-type RAS protein. In some embodiments, a RAS protein is a mutant RAS protein. In some embodiments, a RAS protein (e.g., HRAS, NRAS, or KRAS) comprises a mutation in codon 12 (e.g., G12), codon 13 (e.g., G13), or codon 61 (e.g., Q61). In some embodiments, a KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, a KRAS protein comprises a G12C, G12D, G12S, G12V, G12R, G13D, and/or Q61H mutation. In some embodiments, an NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, an NRAS protein comprises a G12D, G12V, G13D, and/or Q61R mutation. In some embodiments, an HRAS protein comprises a G12C, G12D, G12S, G12V, G12R, G12A, G13D, G13C, G13R, G13S, G13V, Q61K, Q61L, Q61P, Q61R, and/or Q61H mutation. In some embodiments, an HRAS protein comprises a G12V, G13R, and/or Q61R mutation. In some embodiments, a PI3Kα protein is a wild-type PI3Kα protein. In some embodiments, a PI3Kα protein is a mutant PI3Kα protein. In some embodiments, a PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation. In some embodiments, a PI3Kα protein comprises a E542K, E545K, H1047R, and/or H1047L mutation. In some embodiments, provided compounds or compositions are useful in the manufacture of a medicament for treating a disease, disorder, or condition described herein. In some embodiments, provided compounds or compositions are useful in the manufacture of a medicament for treating a cancer or other indication described herein.

In some embodiments, the present disclosure provides compounds or compositions for use in treating a disease, disorder, or condition in a subject in need thereof. In some embodiments, provided compounds or compositions are useful in treating a disease, disorder, or condition associated with or ameliorated by an interaction between a small GTPase (e.g., Rac1. CDC42, or a RAS protein such as KRAS, NRAS, HRAS, RRAS, RRAS2, MRAS, or RIT1) and a PI3Kα protein. In some embodiments, a small GTPase (e.g., Rac1, CDC42, or a RAS protein such as KRAS, NRAS, HRAS, RRAS, RRAS2, MRAS, or RIT1) is capable of binding a RAS-binding domain (RBD) of a PI3Kα protein. In some embodiments, a small GTPase is selected from Rac1, CDC42, and RAS proteins. In some embodiments, a RAS protein is selected from HRAS, NRAS, KRAS, RRAS, RRAS2, MRAS, and RIT1. In some embodiments, a RAS protein is a wild-type RAS protein. In some embodiments, a RAS protein is a mutant RAS protein. In some embodiments, a RAS protein (e.g., HRAS, NRAS, or KRAS) comprises a mutation in codon 12 (e.g., G12), codon 13 (e.g., G13), or codon 61 (e.g., Q61). In some embodiments, a KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, a KRAS protein comprises a G12C, G12D, G12S, G12V, G12R, G13D, and/or Q61H mutation. In some embodiments, an NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation. In some embodiments, an NRAS protein comprises a G12D, G12V, G13D, and/or Q61R mutation. In some embodiments, an HRAS protein comprises a G12C, G12D, G12S, G12V, G12R, G12A, G13D, G13C, G13R, G13S, G13V, Q61K, Q61L, Q61P, Q61R, and/or Q61H mutation. In some embodiments, an HRAS protein comprises a G12V, G13R, and/or Q61R mutation. In some embodiments, a PI3Kα protein is a wild-type PI3Kα protein. In some embodiments, a PI3Kα protein is a mutant PI3Kα protein. In some embodiments, a PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation. In some embodiments, a PI3Kα protein comprises a E542K, E545K, H1047R, and/or H1047L mutation. In some embodiments, provided compounds or compositions are useful for treating a disease, disorder, or condition described herein. In some embodiments, provided compounds or compositions are useful for treating a cancer or other indication as described herein.

In some embodiments, a provided compound or composition is administered as part of a combination therapy. As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously. In some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agents or modalities to a subject receiving the other agent or modality in the combination. For clarity, combination therapy does not require that individual agents be administered together in a single composition (or even necessarily at the same time), although, in some embodiments, two or more agents may be administered together in a combination composition.

In some embodiments, a provided compound or composition is administered to a subject who is receiving or has received one or more additional therapeutic agents (e.g., an anti-cancer agent and/or a therapy to address one or more side effects of such anti-cancer therapy, or otherwise to provide palliative care). Exemplary anti-cancer agents include, but are not limited to, an alkylating agent, an antimitotic, a checkpoint inhibitor, an anti-metabolite, a plant alkaloid, a terpenoid, a cytotoxic agent, an antibiotic, a topoisomerase inhibitor, an aromatase inhibitor, an angiogenesis inhibitor, an anti-steroid, an anti-androgen, an mTOR inhibitor, monoclonal antibodies, a kinase inhibitor, a HIF2α inhibitor, or a tyrosine kinase inhibitor. An alkylating agent may be, for example, armustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, or cyclophosphamide (ENDOXAN). An anti-metabolite may be, for example, cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEY), methotrexate (RHEUMATREX), or raltitrexed. An antimitotic may be, for example, a taxane such as docetaxel (TAXITERE) or paclitaxel (ABRAXANE, TAXOL), or a vinca alkaloid such as vincristine (ONCOVIN), vinblastine, vindesine, or vinorelbine (NAVELBINE). A checkpoint inhibitor may be an anti-PD-1 or anti-PD-L1 antibody such as pembrolizumab (KEYTRUDA), nivolumab (OPDIVO), MEDI4736, or MPDL3280A; anti-CTLA-4 antibody ipilimumab (YERVOY); or an agent that targets LAG3 (lymphocyte activation gene 3 protein), KIR (killer cell immunoglobulin-like receptor), 4-1BB (tumor necrosis factor receptor superfamily member 9), TIM3 (T-cell immunoglobulin and mucin-domain containing-3), or 0X40 (tumor necrosis factor receptor superfamily member 4). A topoisomerase inhibitor may be, for example, camptothecin (CTP), irinotecan (CAMPTOSAR), topotecan (HYCAMTIN), teniposide (VUMON), or etoposide (EPOSIN). A cytotoxic antibiotic may be, for example, actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOYANTRONE), or plicamycin. An aromatase inhibitor may be, for example, aminoglutethimide, anastrozole (ARIMIDEX), letrozole (FEMARA), vorozole (RIYIZOR), or exemestane (AROMASIN). An angiogenesis inhibitor may be, for example, genistein, sunitinib (SUTENT), or bevacizumab (AYASTIN). An anti-steroid or anti-androgen may be, for example, aminoglutethimide (CYTADREN), bicalutamide (CASODEX), cyproterone, flutamide (EULEXIN), or nilutamide (NILANDRON). A tyrosine kinase inhibitor may be, for example, imatinib (GLEEVEC), erlotinib (TARCEVA), afatinib (GILOTRIF), lapatinib (TYKERB), sorafenib (NEXAVAR), or axitinib (INLYTA). An mTOR inhibitor may be, for example, everolimus, temsirolimus (TORISEL), or sirolimus. Monoclonal antibody may be, for example, trastuzumab (HERCEPTIN) or rituximab (RITUXAN). A kinase inhibitor may be, for example, a BRAF inhibitor, MEK inhibitor, or a KRAS inhibitor (e.g., KRAS G12C inhibitor, such as sotorasib, adagrasib, or BBO-8520). Additional examples of agents that may be useful in combination with a compound provided herein include, but are not limited to, amsacrine; Bacillus Calmette-Guerin (B—C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,r′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

### EXEMPLARY EMBODIMENTS

The following numbered embodiments, while non-limiting, are exemplary of certain aspects of the present disclosure:
1. A Compound of Formula I:

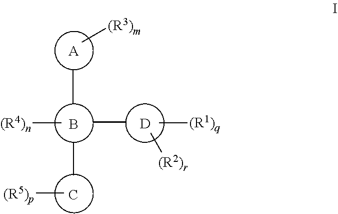

or a salt (e.g., pharmaceutically acceptable salt) thereof, wherein:
Ring A is selected from phenyl and a 5- to 6-membered heteroaryl ring having 1-3 heteroatoms independently selected from nitrogen, oxygen, and sulfur;
Ring B is selected from phenyl, a 6-membered heteroaryl ring having 1-2 nitrogen atoms, and a 9- to 10-membered bicyclic ring that comprises at least one 5- or 6-membered heteroaryl ring;
Ring C is selected from phenyl; a 5- to 6-membered heteroaryl ring having 1-3 heteroatoms independently selected from nitrogen, oxygen, and sulfur; a 5- to 6-membered heterocyclic ring having 1-2 heteroatoms independently selected from nitrogen, oxygen, and sulfur; and a 9- to 10-membered spirofused heterocyclic ring having 1-3 heteroatoms independently selected from nitrogen, oxygen, and sulfur, wherein each of the phenyl, heteroaryl, and heterocyclic rings is optionally fused to Ring E;
Ring D is selected from phenyl and a 5- to 6-membered heteroaryl ring having 1-3 heteroatoms independently selected from nitrogen, oxygen, and sulfur, wherein each of the phenyl and heteroaryl rings is optionally fused to Ring F;
Ring E is selected from a 5- to 6-membered carbocyclic ring; a 5- to 7-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring E is substituted by s instances of R^{5′};
Ring F is selected from phenyl; a 5- to 6-membered carbocyclic ring; a 5- to 6-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring F is substituted by u instances of -L-W;
R¹ is selected from -L-W, Ring D′, or a bivalent C₁₋₆ aliphatic chain substituted with Ring D′;
each L is independently a bivalent straight or branched C₁₋₈ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—;
each W is independently hydrogen, halogen, —CN, or a 3-10 membered monocyclic or bicyclic, saturated, partially unsaturated, or aryl ring having 0-3 heteroatoms independently selected from nitrogen, oxygen, or sulfur;
each X is independently halogen or —CN;
each Ring D′ is independently a 4- to 6-membered carbocyclic ring or a 4- to 6-membered heterocyclic ring having 1-2 heteroatoms independently selected from nitrogen, oxygen, and sulfur, wherein Ring D′ is substituted with t instances of -L-W;
each R² is independently selected from oxo, halogen, —CN, —OR, and C₁₋₆ alkyl;
each R³ is independently selected from oxo, halogen, —CN, —OR, —O(CH₂)ᵥCy, —OCH₂CH₂OR, —CH₂CH₂OR, C₁₋₆ aliphatic, and C₁₋₆ haloaliphatic;
each Cy is independently a 5- to 6-membered heteroaryl ring having 1-3 heteroatoms independently selected from nitrogen, oxygen, and sulfur; a 3- to 6-membered carbocyclic ring; or a 4- to 6-membered heterocyclic ring having 1-2 heteroatoms independently selected from nitrogen, oxygen, and sulfur, wherein Cy is substituted with 0-2 instances of R⁶;
each R⁴ is independently selected from halogen and C₁₋₆ aliphatic;
each of R⁵ and R^{5′} is independently selected from oxo, =NH, —CN, halogen, —OR, —N(R)₂, —SR, —C(O)R, —N(R)C(O)R, —(CH₂)ₓC(O)N(R)₂, —OC(O)R, —C(O)OR, —SO₂R, —N(R)SO₂R, —SO₂N(R)₂, —(CH₂)ₓCy, and C₁₋₆ aliphatic, wherein C₁₋₆ aliphatic is unsubstituted or substituted with one or more halogen and —OR;
each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and a group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
each R is independently hydrogen; C₁₋₆ aliphatic; C₁₋₆ haloaliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
m is 0, 1, 2, or 3;
n is 0, 1, or 2;
p is 0, 1, or 2;
q is 0 or 1;
r is 0, 1, or 2;
s is 0, 1, or 2;
t is 0, 1, or 2;
u is 0 or 1;
each v is independently 0, 1, or 2; and
each x is independently 0, 1, or 2.
2. The compound according to embodiment 1, wherein L is a bivalent straight or branched C₁₋₈ aliphatic chain, wherein one or two methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.
3. The compound according to embodiment 1 or 2, wherein L is a bivalent straight or branched C₁₋₈ aliphatic chain, wherein one or two methylene units of the aliphatic chain are optionally and independently replaced by a group selected from —N(R)—, —O—, C(O)—, —C(O)N(R)—, and —N(R)C(O)—.
4. The compound according to any one of embodiments 1-3, wherein L is a bivalent straight or branched C1-s aliphatic chain having one or more units of unsaturation, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.
5. The compound according to embodiment 4, wherein L is a bivalent straight or branched C₁₋₄ aliphatic chain having one or more units of unsaturation, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.
6. The compound according to embodiment 5, wherein L is a bivalent straight or branched C₁₋₄ aliphatic chain having one double bond, wherein one or two methylene units of the aliphatic chain are optionally and independently replaced by a group selected from —N(R)—, —O—, —C(O)—, —SO₂—, —C(O)N(R)—, —N(R)C(O)—, —C(O)O—, —OC(O)—, —SO₂N(R)—, and —N(R)SO₂—.
7. The compound according to embodiment 6, wherein L is a bivalent straight or branched C₁₋₄ aliphatic chain having one double bond, wherein one or two methylene units of the aliphatic chain are optionally and independently replaced by a group selected from —N(R)—, —C(O)—, and —N(R)C(O)—.
8. The compound according to embodiment 1, wherein L is selected from —C(O)CClF—, —C(O)CH═CH—, —N(R)C(O)CH═CH—, —C(O)C(≡CH₂)—, —C(O)CH═CH—CH₂—C(O)CH═CHCH₂OCH₂—, —C(O)CH═CHCH₂N(R)—, —CH₂N(R)C(O)CH═CH—, and —CH₂CH₂N(R)C(O)CH═CH—.
9. The compound according to embodiment 8, wherein L is selected from —C(O)CClF—, —C(O)CH═CH—, —NHC(O)CH═CH—, —C(O)C(≡CH₂)—, —C(O)CH═CH—CH₂—, —C(O)CH═CHCH₂OCH₂—, —C(O)CH═CHCH₂N(CH₃)—, —CH₂NHC(O)CH═CH—, and —CH₂CH₂NHC(O)CH═CH—.
10. The compound according to embodiment 1 or 2, wherein L is a bivalent straight or branched C₁₋₈ alkyl chain, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.
11. The compound according to embodiment 1, wherein L is selected from —N(R)C(O)CH₂CH₂—, —N(R)C(O)CH(CH₃)CH₂—, —OCH(CH₃)CH₂—, —C(O)—, —C(O)CH₂CH₂—, —C(O)N(R)CH(CH₃)CH₂—, —C(O)N(R)CH₂CH(CH₃)CH₂—, —CH₂—, —CH(CH₃)CH₂—, —CH₂C(CH₃)₂CH₂—, —CH₂C(O)—, —CH₂C(O)N(R)C(CH₃)₂CH₂—, —CH₂CH₂C(O)N(R)C(CH₃)₂CH₂—, —CH₂N(R)—, —CH₂N(R)C(O)—, —CH₂N(R)C(O)CH₂CH₂—, —CH₂N(R)C(O)CH(CH₃)CH₂—, —CH₂N(R)C(O)C(CH₃)₂CH₂—, —CH₂N(R)C(O)CH₂C(CH₃)₂CH₂—, —CH₂CH₂N(R)C(O)CH(CH₃)CH₂—, and —CH₂O—.
12. The compound according to embodiment 11, wherein L is selected from —N(H)C(O)CH₂CH₂—, —N(H)C(O)CH(CH₃)CH₂—, —OCH(CH₃)CH₂—, —C(O)—, —C(O)CH₂CH₂—, —C(O)N(H)CH(CH₃)CH₂—, —C(O)N(H)CH₂CH(CH₃)CH₂—, —CH₂—, —CH(CH₃)CH₂—, —CH₂C(CH₃)₂CH₂—, —CH₂C(O)—, —CH₂C(O)N(H)C(CH₃)₂CH₂—, —CH₂CH₂C(O)N(H)C(CH₃)₂CH₂—, —CH₂N(H)—, —CH₂N(H)C(O)—, —CH₂N(H)C(O)CH₂CH₂—, —CH₂N(H)C(O)CH(CH₃)CH₂—, —CH₂N(H)C(O)C(CH₃)₂CH₂—, —CH₂N(H)C(O)CH₂C(CH₃)₂CH₂—, —CH₂CH₂N(H)C(O)CH(CH₃)CH₂—, and —CH₂O—.
13. The compound according to any one of embodiments 1-12, wherein W is hydrogen.
14. The compound according to any one of embodiments 1-12, wherein W is a halogen.
15. The compound according to embodiment 1, wherein -L-W is selected from —CH₃, —CH(CH₃)₂, —CH₂C(CH₃)₃, —CH₂OH, —CH₂NH₂, —OCH(CH₃)₂,

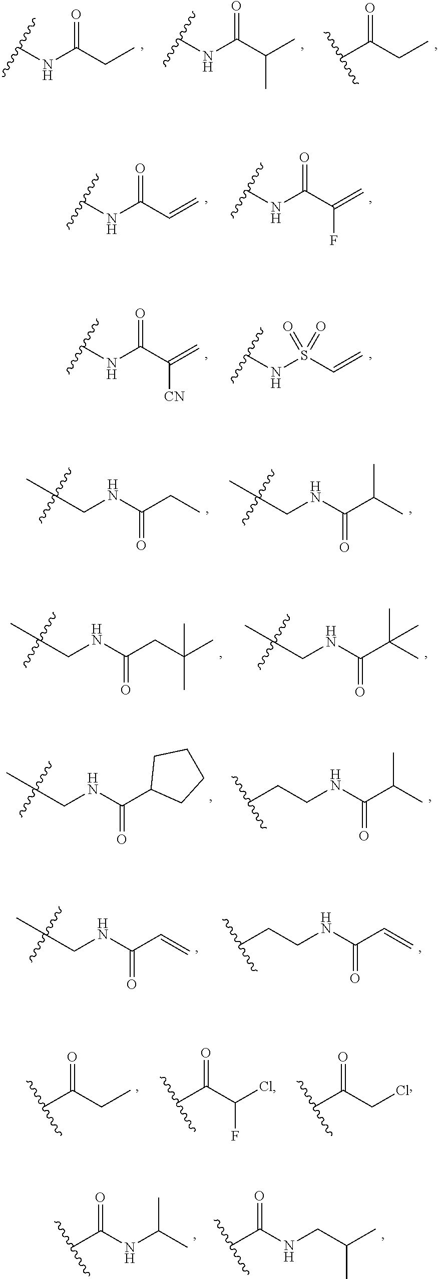

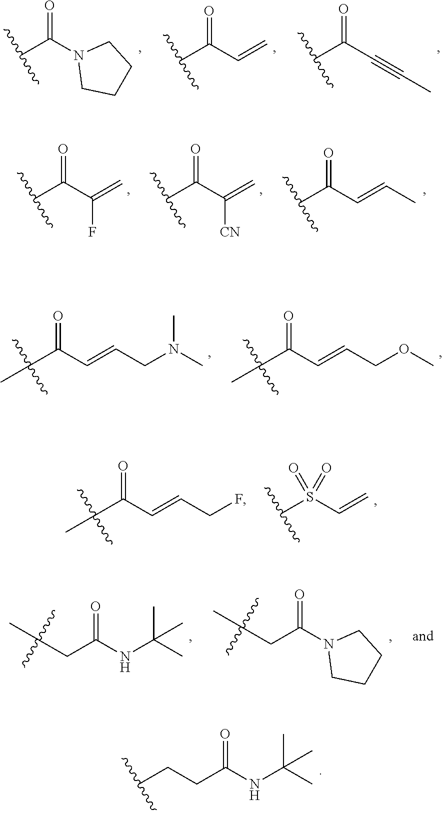

16. The compound according to embodiment 1, wherein -L-W is selected from —C(O)CH═CH₂, —C(O)CF=CH₂, —NHC(O)CF=CH₂, and —NHC(O)CH═CH₂.
17. The compound according to embodiment 1, wherein R¹ is selected from —CH₃, —CH(CH₃)₂, —CH₂C(CH₃)₃, —CH₂NH₂, —CH₂OH, —OCH(CH₃)₂,

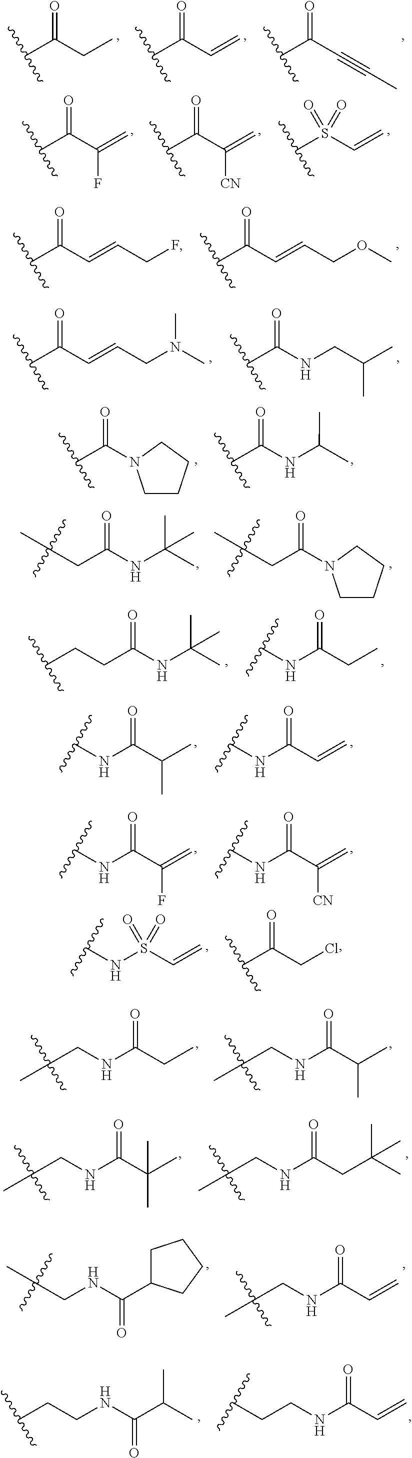

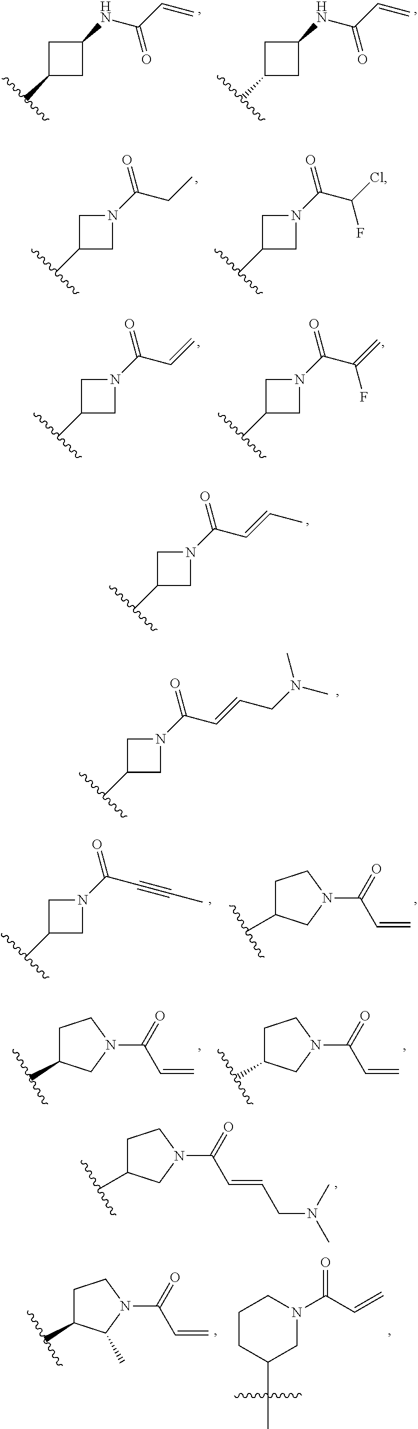

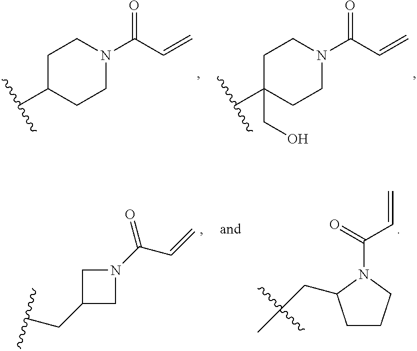

18. The compound according to any one of embodiments 1-17, wherein Ring B is a 9- to 10-membered bicyclic ring which comprises at least one 5- or 6-membered heteroaryl ring comprising at least one nitrogen atom.
19. The compound according to any one of embodiments 1-18, wherein Ring B, substituted with n instances of R⁴, is selected from

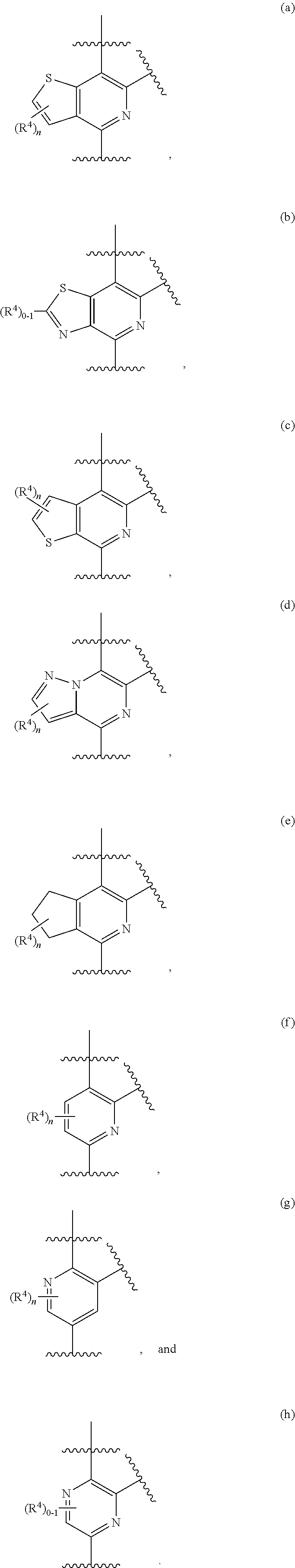

20. The compound according to any one of embodiments 1-19, wherein the compound is selected from Formulae I-a, I-b, I-c, I-d, I-e, I-f, I-g, and I-h:

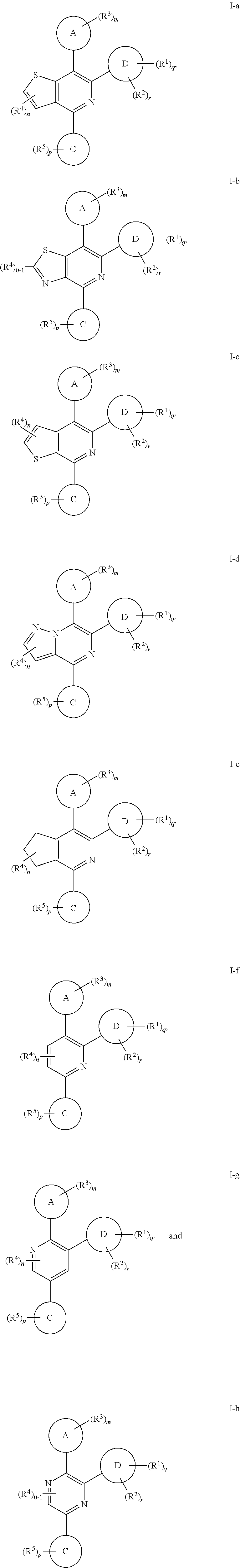

or a pharmaceutically acceptable salt thereof
21. The compound according to any one of embodiments 1-20, wherein each R⁴ is halogen.
22. The compound according to any one of embodiments 1-20, wherein n is 0.
23. The compound according to any one of embodiments 1-22, wherein Ring A is phenyl.
24. The compound according to any one of embodiments 1-23, wherein Ring A is

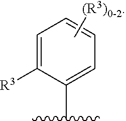

25. The compound according to embodiment 24, wherein Ring A is selected from

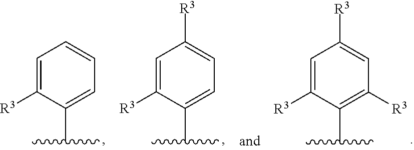

26. The compound according to embodiment 25, wherein Ring A is selected from

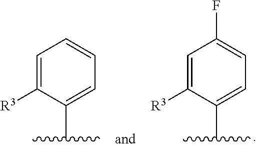

27. The compound according to embodiment 26, wherein R³ is selected from halogen, —OR, —O(CH₂)ᵥCy, and —OCH₂CH₂OR.
28. The compound according to any one of embodiments 1-27, wherein Ring A, substituted with m instances of R³, is selected from

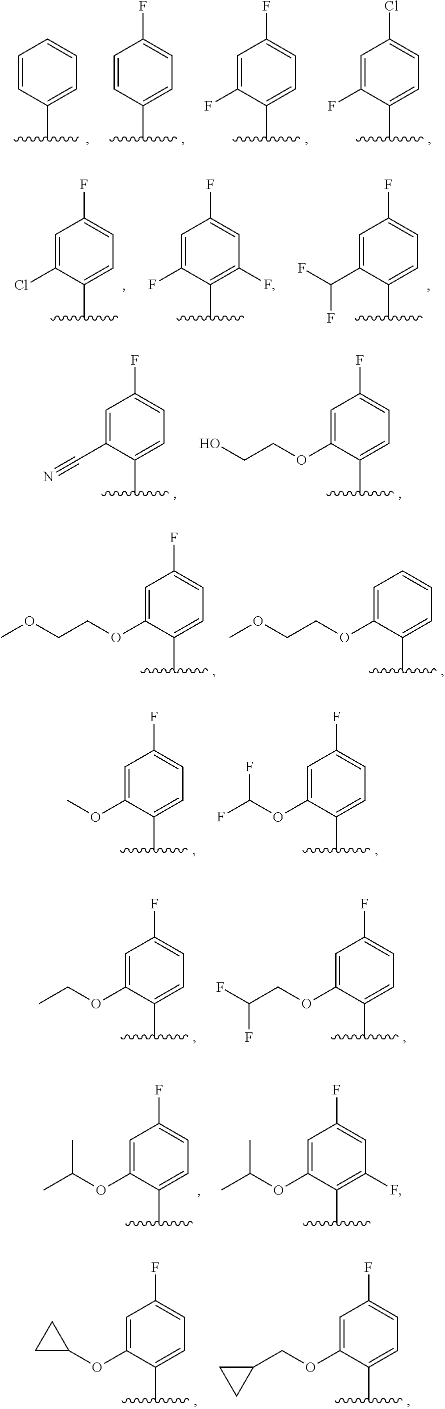

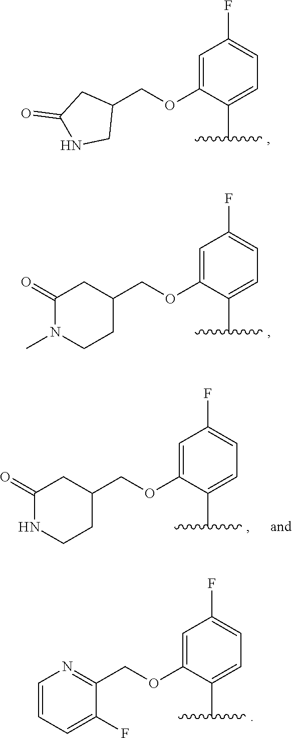 [SMILES: *c1ccc(F)cc1OCC1CNC(=O)C1 ; *c1ccc(F)cc1OCC1CCN(C)C(=O)C1 ; *c1ccc(F)cc1OCC1CCNC(=O)C1 ; *c1ccc(F)cc1OCc1ncccc1F]

29. The compound according to any one of embodiments 1-22, wherein Ring A is a 5- to 6-membered heteroaryl ring having 1-3 heteroatoms independently selected from nitrogen, oxygen, and sulfur.
30. The compound according to embodiment 29, wherein Ring A substituted with m instances of R³ is selected from

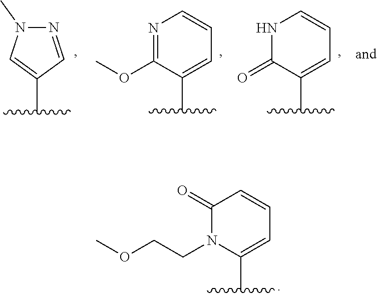

31. The compound according to any one of embodiments 1-30, wherein m is 1, 2, or 3.
32. The compound according to any one of embodiments 1-31, wherein at least one R³ is selected from halogen, —OR, —O(CH₂)ᵥCy, and —OCH₂CH₂OR.
33. The compound according to any one of embodiments 1-32, wherein Ring C is phenyl.
34. The compound according to any one of embodiments 1-32, wherein Ring C is a 5- to 6-membered heteroaryl ring having 1-3 heteroatoms independently selected from nitrogen, oxygen, and sulfur.
35. The compound according to any one of embodiments 1-32, wherein Ring C is a 5- to 6-membered heterocyclic ring having 1-2 heteroatoms independently selected from nitrogen, oxygen, and sulfur.
36. The compound according to any one of embodiments 1-32, wherein Ring C is a 9- to 10-membered spirofused heterocyclic ring having 1-3 heteroatoms independently selected from nitrogen, oxygen, and sulfur.
37. The compound according to any one of embodiments 1-32, wherein Ring C, substituted with p instances of R⁵, is selected from

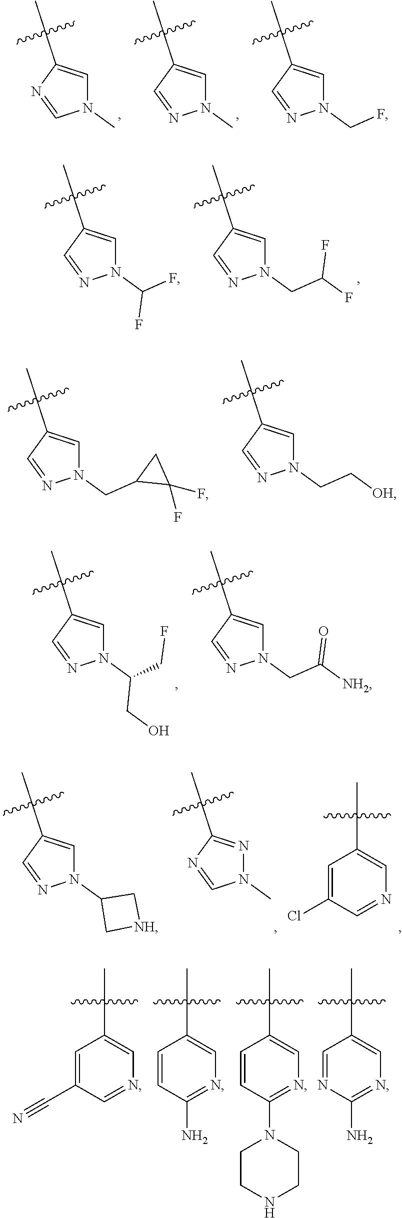

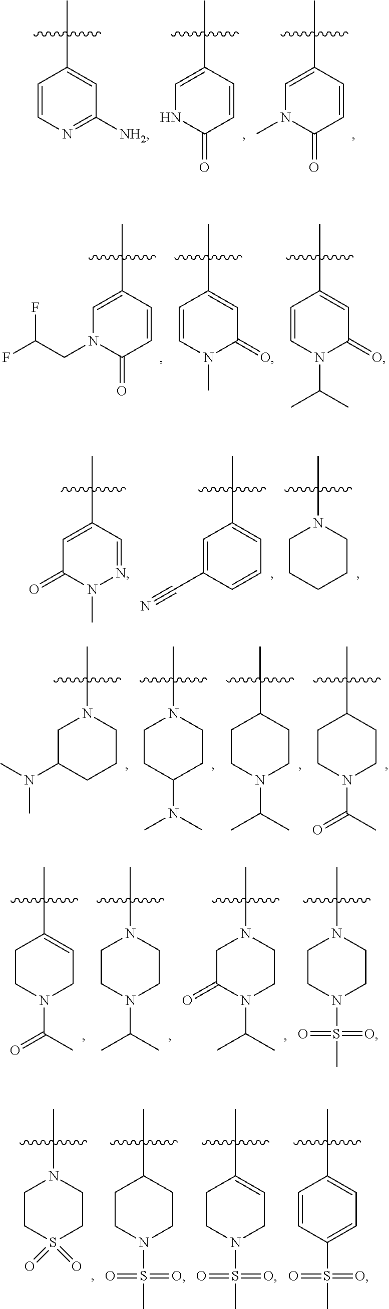

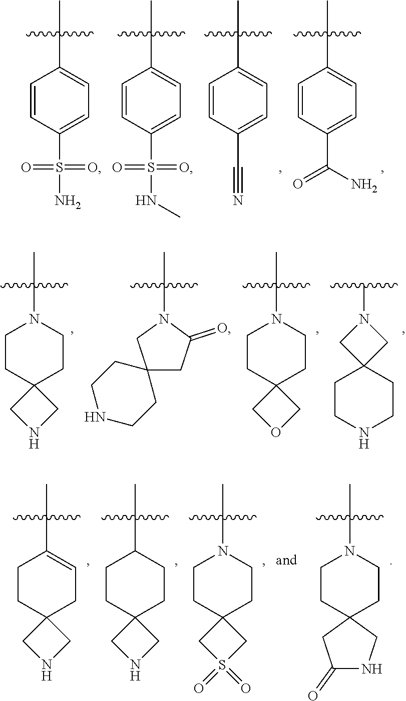

38. The compound according to any one of embodiments 1-36, wherein Ring C is fused to Ring E.
39. The compound according to embodiment 38, wherein the compound is selected from Formulae I-a-i, I-b-i I-c-i, I-d-i, I-e-i, I-f-i, I-g-i, and I-h-i:

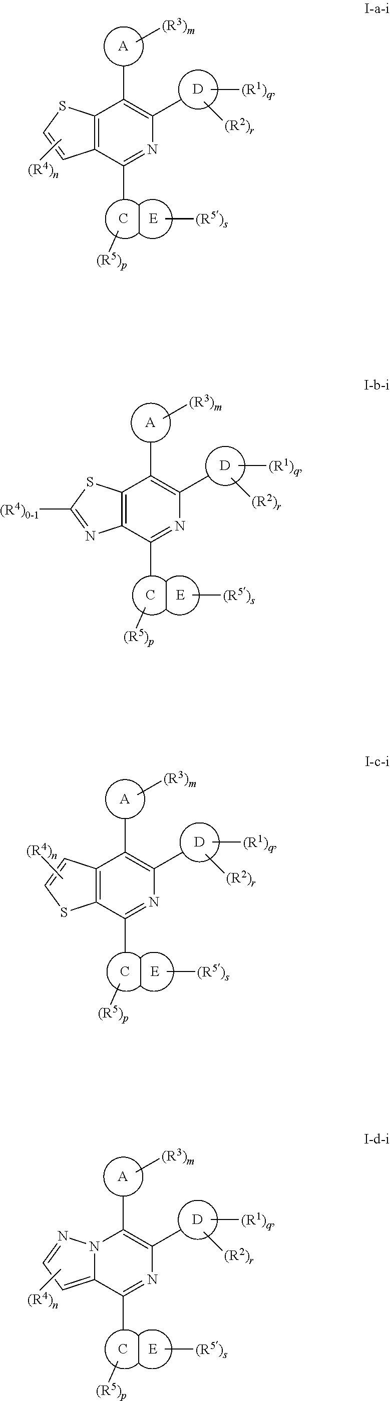

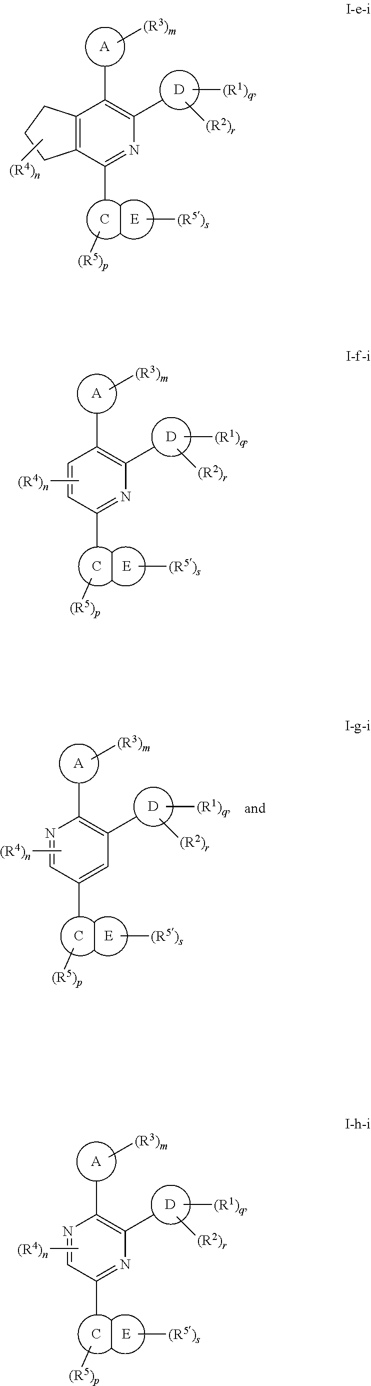

or a pharmaceutically acceptable salt thereof.
40. The compound according to embodiment 39, wherein

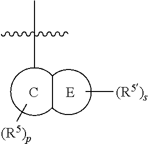

is selected from

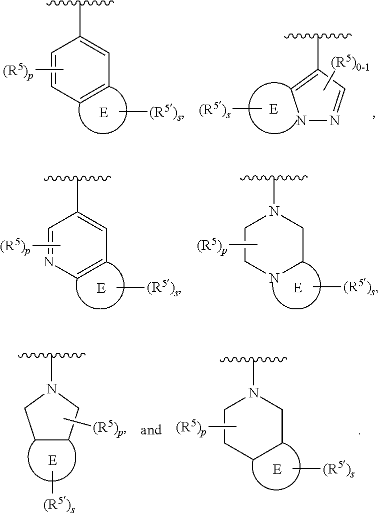

41. The compound according to any one of embodiments 38-40, wherein Ring E is a 5- to 7-membered heterocyclic ring having 1-2 heteroatoms independently selected from nitrogen, oxygen, and sulfur, wherein Ring E is substituted by s instances of R^{5′}.
42. The compound according to embodiment 41, wherein Ring E, substituted with s instances of R^{5′}, is selected from

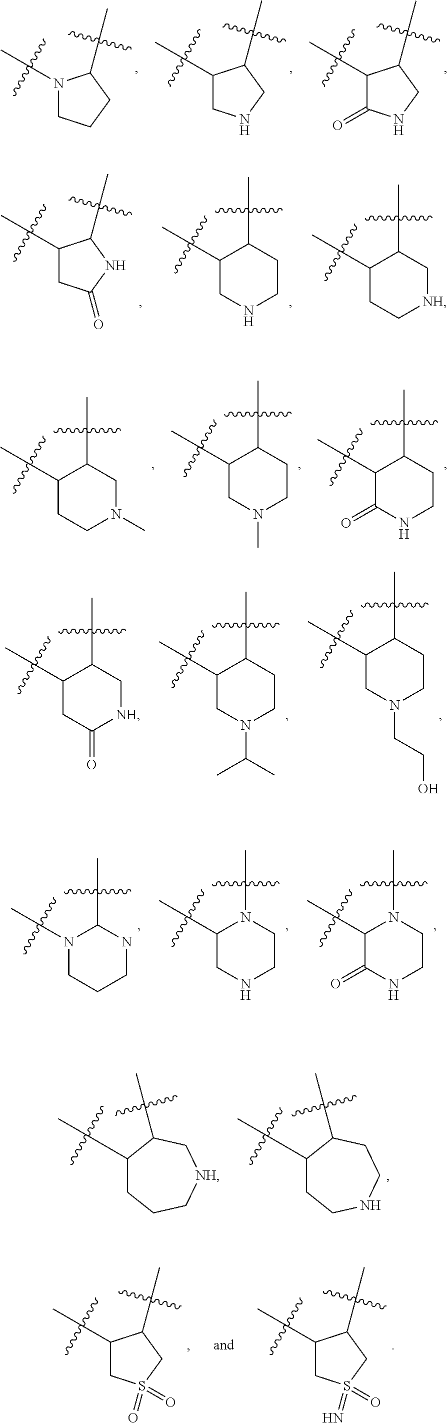

43. The compound according to any one of embodiments 38-40, wherein Ring E is a 5- to 6-membered heteroaryl ring having 1-3 heteroatoms independently selected from nitrogen, oxygen, and sulfur, wherein Ring E is substituted by s instances of R^{5′}.
44. The compound according to embodiment 19, wherein Ring E, substituted with s instances of R^{5′} is selected from

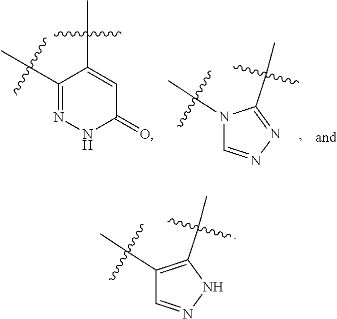 [SMILES: *c1cc(=O)[nH]nc1* ; *c1nncn1* ; *c1cn[nH]c1*]

45. The compound according to any one of embodiments 38-40, wherein Ring E is a 5- to 6-membered carbocyclic ring substituted by s instances of R^{5′}.
46. The compound according to embodiment 45, wherein Ring E, substituted with s instances of R^{5′} is selected from

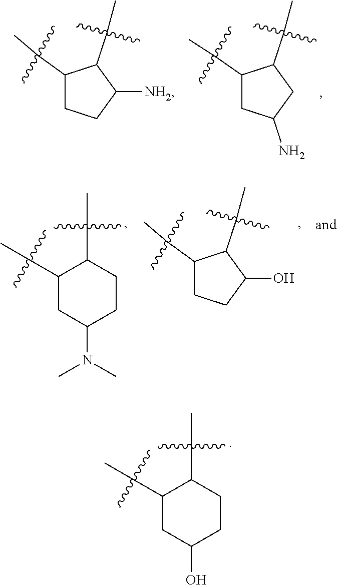 [SMILES: *C1CCC(N)C1* ; *C1CC(N)CC1* ; *C1CCC(N(C)C)CC1* ; *C1CCC(O)C1* ; *C1CCC(O)CC1*]

47. The compound according to any one of embodiments 38-40, wherein

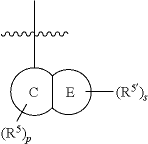

is selected from

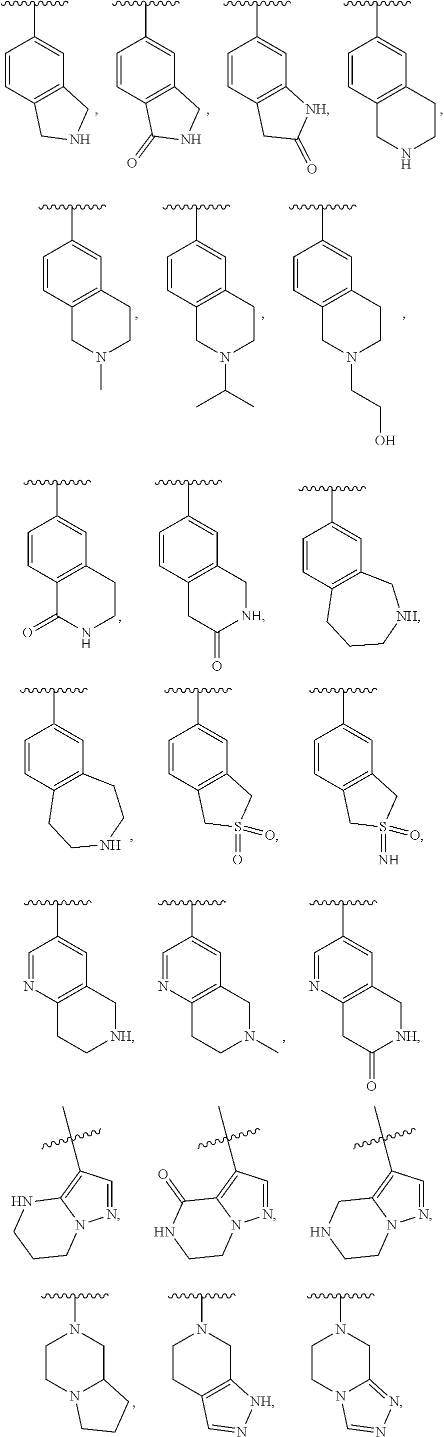

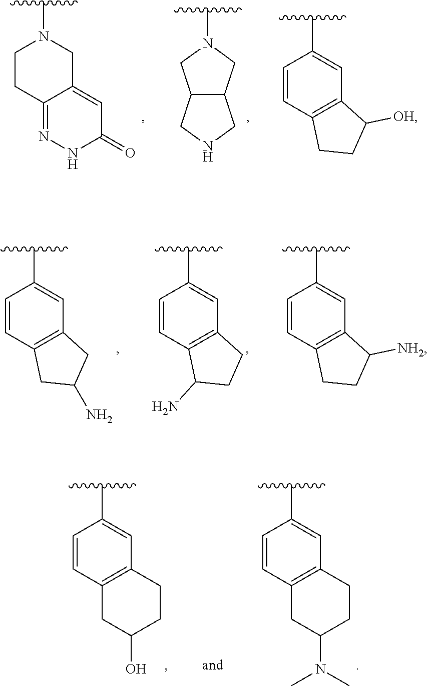

48. The compound according to any one of embodiments 1-47, wherein p is 1 or 2.
49. The compound according to any one of embodiments 1-48, wherein each R⁵ is independently selected from oxo, —CN, halogen, —N(R)₂, —(CH₂)ₓC(O)N(R)₂, —SO₂R, —SO₂N(R)₂, —(CH₂)ₓCy, and C₁₋₆ aliphatic, wherein C₁₋₆ aliphatic is unsubstituted or substituted with one or more halogen and —OR.
50. The compound according to any one of embodiments 1-49, wherein each R^{5′} is independently selected from oxo, =NH, —OR, —N(R)₂, and C₁₋₆ aliphatic, wherein C₁₋₆ aliphatic is unsubstituted or substituted with one or more halogen and —OR.
51. The compound according to any one of embodiments 1-50, wherein Ring D is phenyl.
52. The compound according to any one of embodiments 1-50, wherein Ring D is a 5- to 6-membered heteroaryl ring having 1-3 heteroatoms independently selected from nitrogen, oxygen, and sulfur.
53. The compound according to any one of embodiments 1-52, wherein Ring D, substituted with r instances of R² and q instances of R¹, is selected from

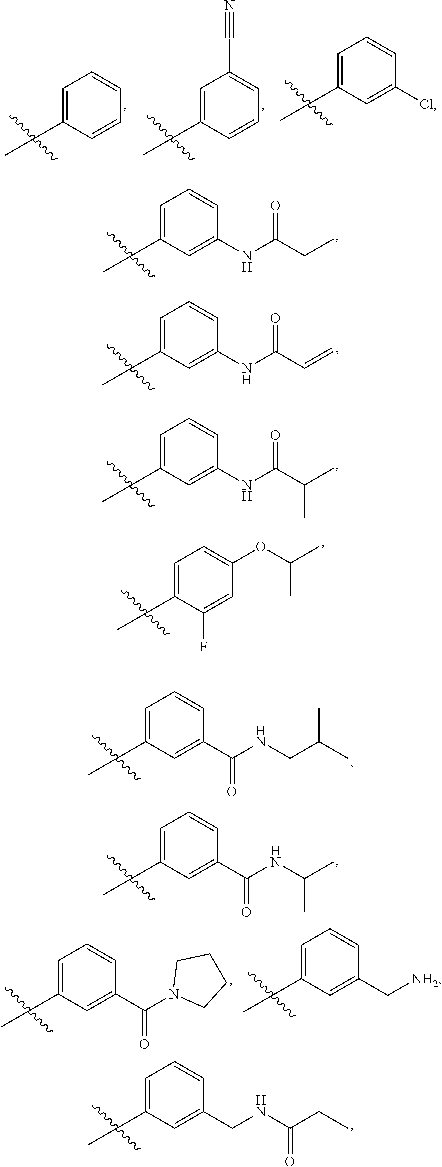

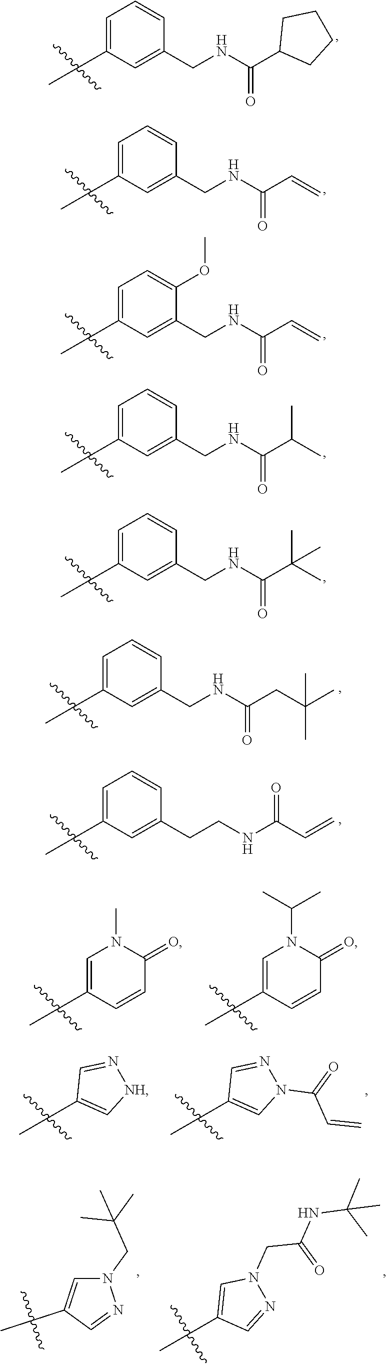

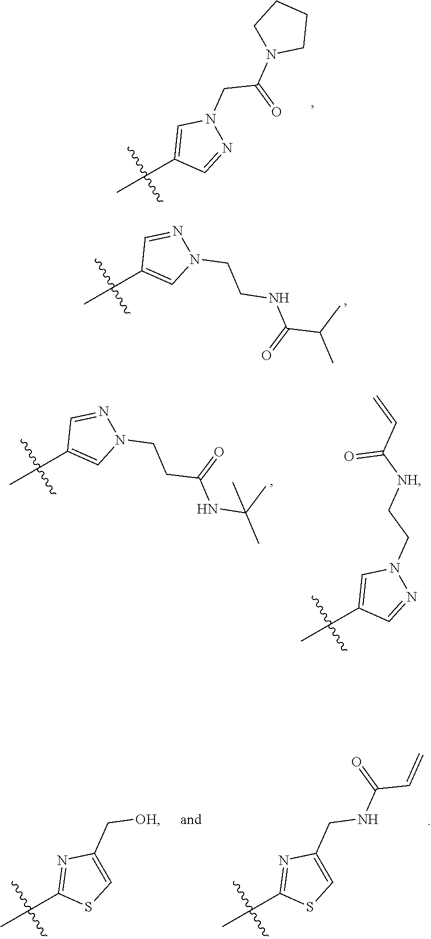

54. The compound according to any one of embodiments 1-52, wherein Ring D is fused to Ring F.
55. The compound according to embodiment 54, wherein q is 0.
56. The compound according to any one of embodiments 1-52, 54, and 55, wherein the compound is selected from Formulae I-a-ii, I-b-ii, I-c-ii, I-d-ii, I-e-ii, I-f-ii, I-g-ii, and I-h-ii:

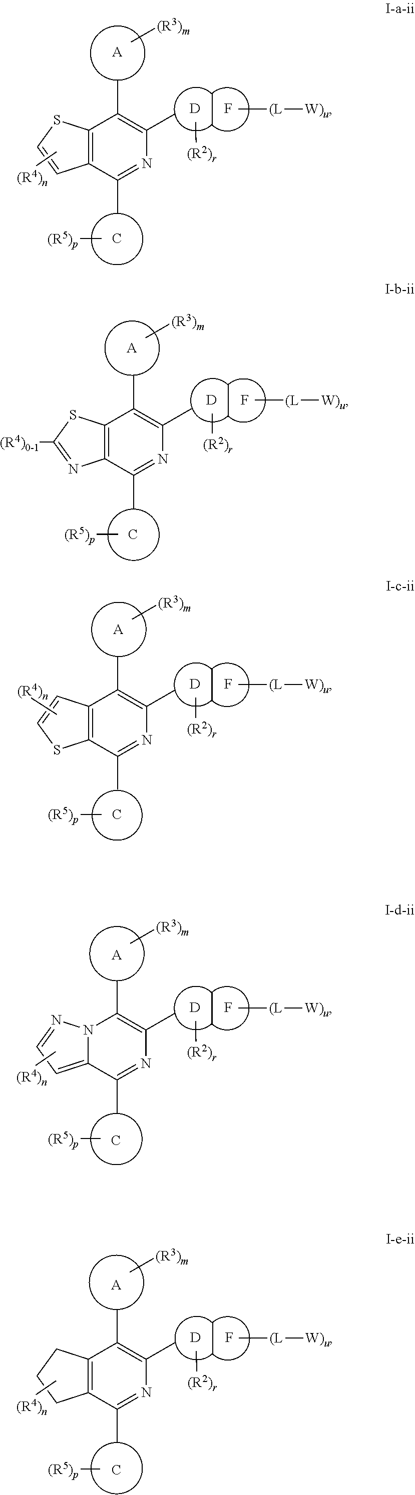

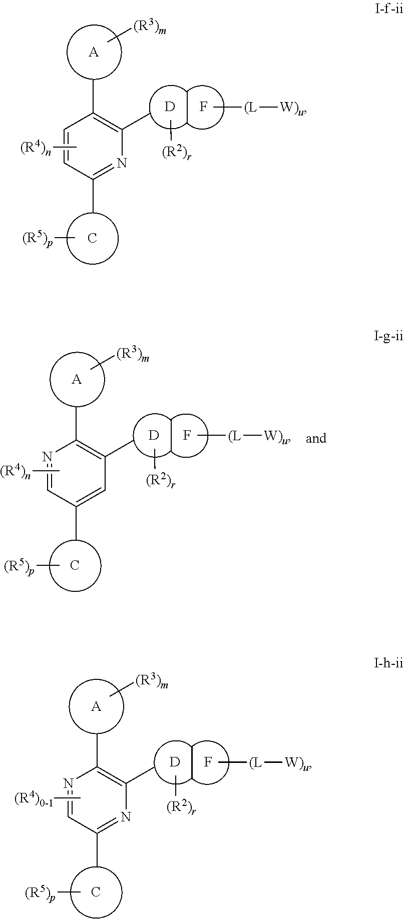

or a pharmaceutically acceptable salt thereof.
57. The compound according to embodiment 56, wherein

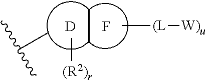

is selected from

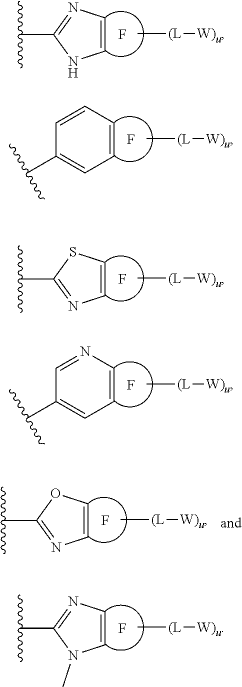

58. The compound according to any one of embodiments 1-52 and 54-57, wherein Ring F is phenyl substituted by u instances of -L-W.
59. The compound according to embodiment 58, wherein Ring F, substituted with u instances of -L-W, is selected from

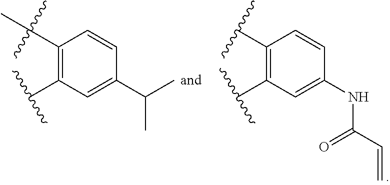 [SMILES: *c1ccc(C(C)C)cc1* ; *c1ccc(NC(=O)C=C)cc1*]

60. The compound according to embodiment 56, wherein

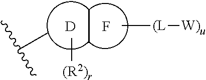

is selected from

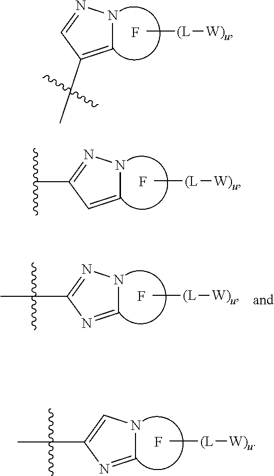

61. The compound according to any one of embodiments 1-52, 54-57, and 60, wherein Ring F is a 5- to 6-membered carbocyclic ring substituted by u instances of -L-W.
62. The compound according to embodiment 61, wherein Ring F, substituted by u instances of -L-W, is

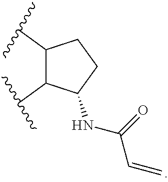 [SMILES: *C1CC[C@H](NC(=O)C=C)C1*]

63. The compound according to any one of embodiments 1-52, 54-57, and 60, wherein Ring F is a 5- to 6-membered heterocyclic ring having 1-2 heteroatoms independently selected from nitrogen, oxygen, and sulfur, wherein Ring F is substituted by u instances of -L-W.
64. The compound according to embodiment 63, wherein Ring F, substituted by u instances of -L-W, is selected from

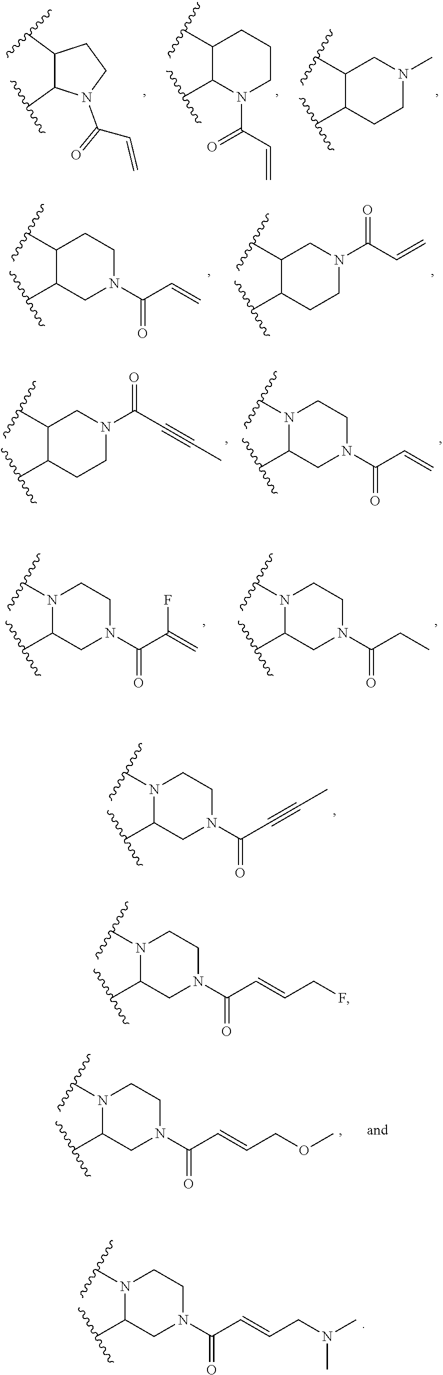

65. The compound according to any one of embodiments 1-52, 54-57, and 60, wherein Ring F is a 5- to 6-membered heteroaryl ring having 1-3 heteroatoms independently selected from nitrogen, oxygen, and sulfur, wherein Ring F is substituted by u instances of -L-W.
66. The compound according to embodiment 65, wherein Ring F, substituted by u instances of -L-W, is

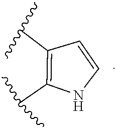 [SMILES: *c1cc[nH]c1*]

67. The compound according to embodiment 56, wherein

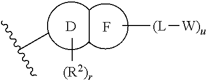

is selected from

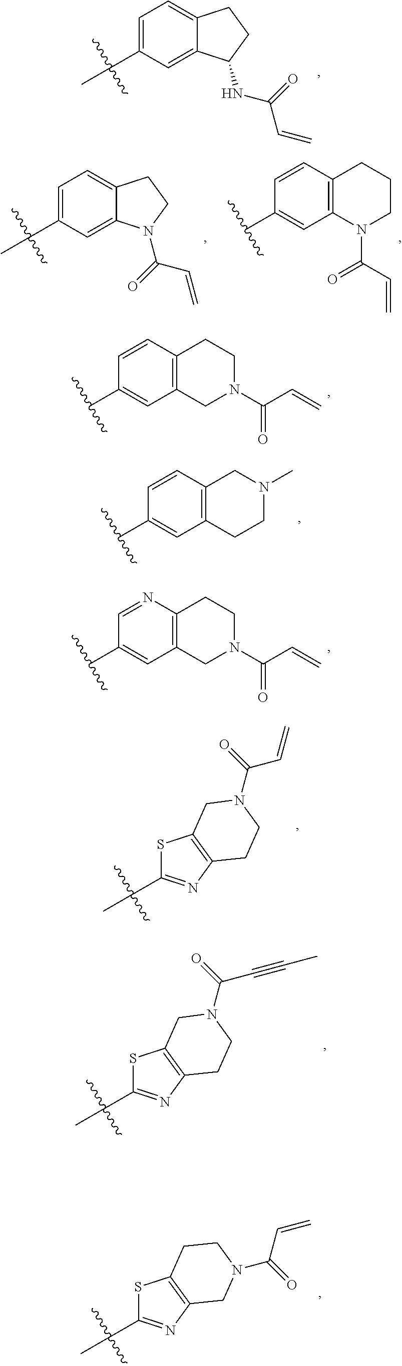

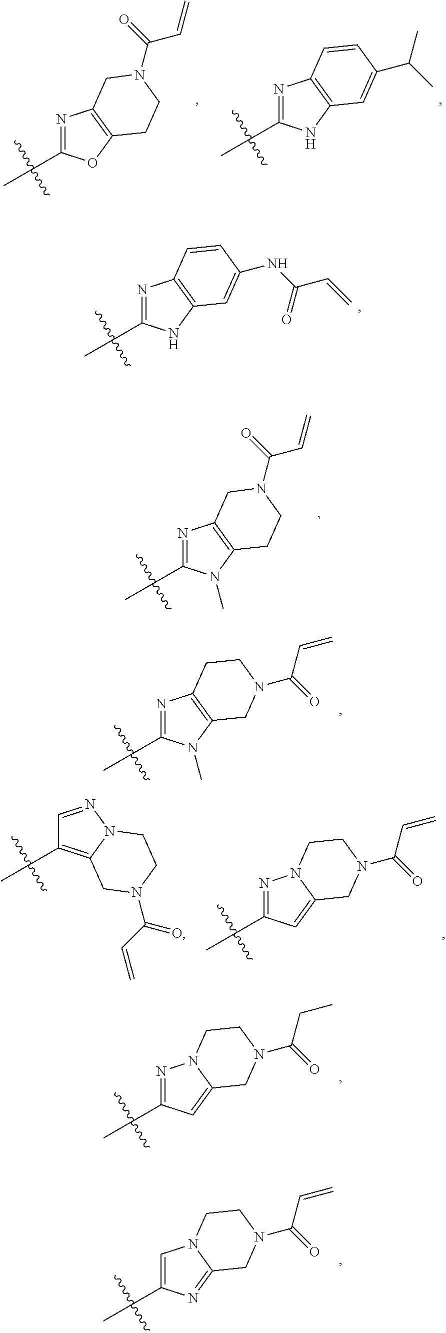

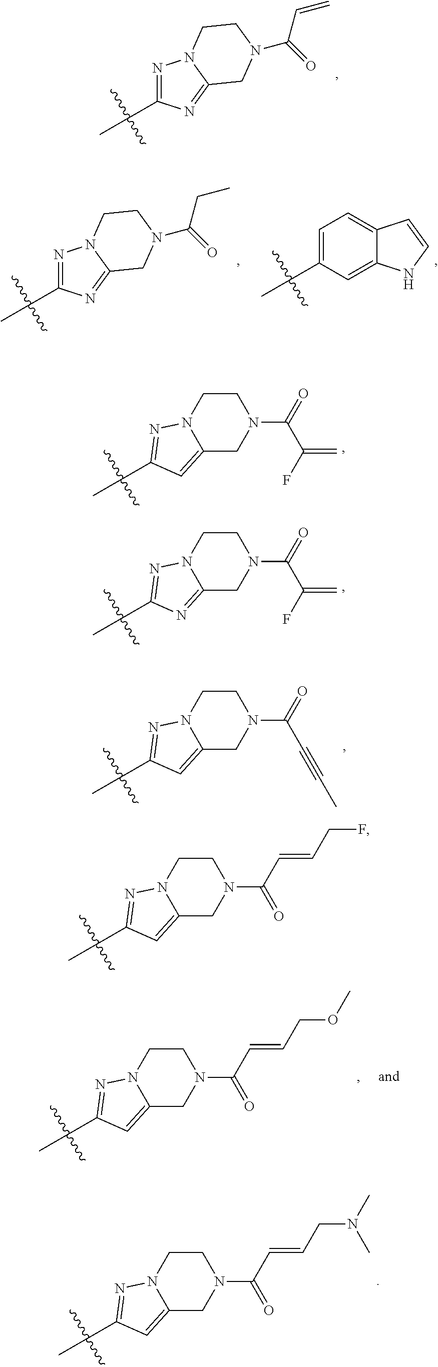

68. The compound according to any one of embodiments 1-52, wherein R¹ is Ring D′.
69. The compound according to embodiment 68, wherein Ring D is not fused to Ring F.
70. The compound according to any one of embodiments 1-52, wherein the compound is selected from Formulae I-a-iii, I-b-iii, I-c-iii, I-d-iii, I-e-iii, I-f-iii, I-g-iii, and I-h-iii:

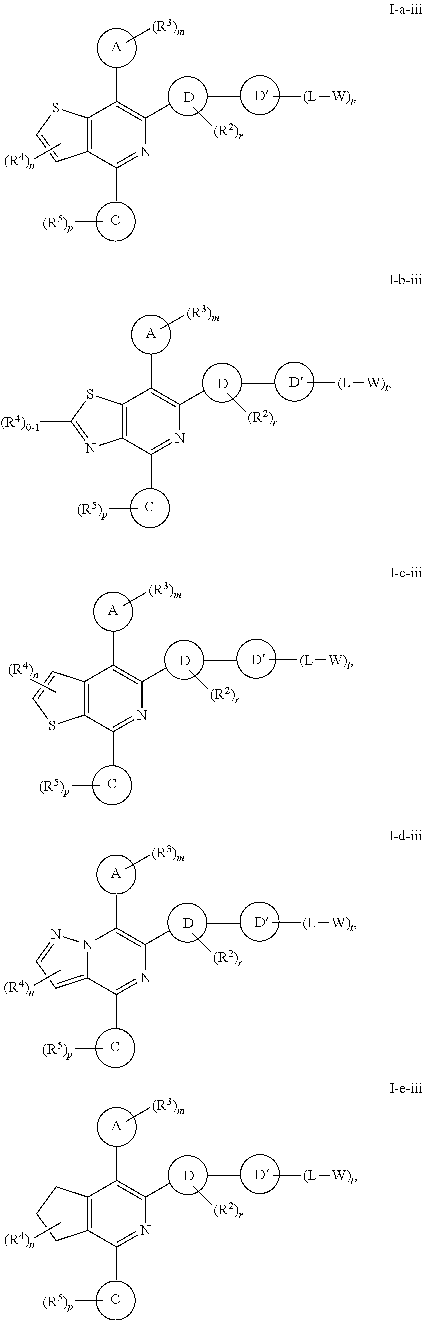

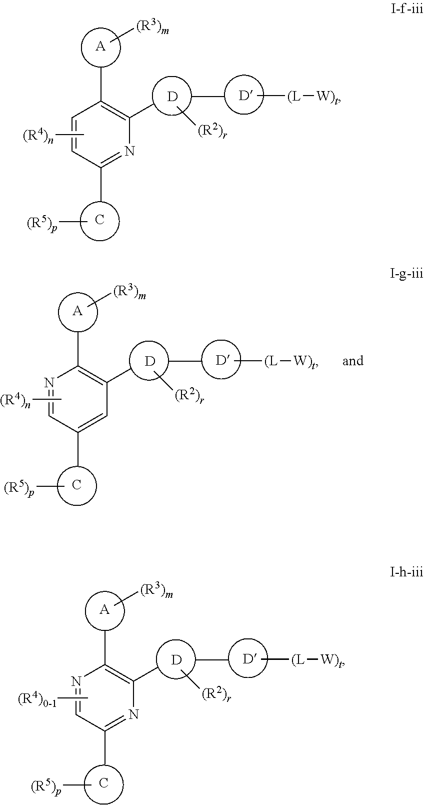

or a pharmaceutically acceptable salt thereof.
71. The compound according to embodiment 70, wherein Ring D, substituted with r instances of R², is selected from

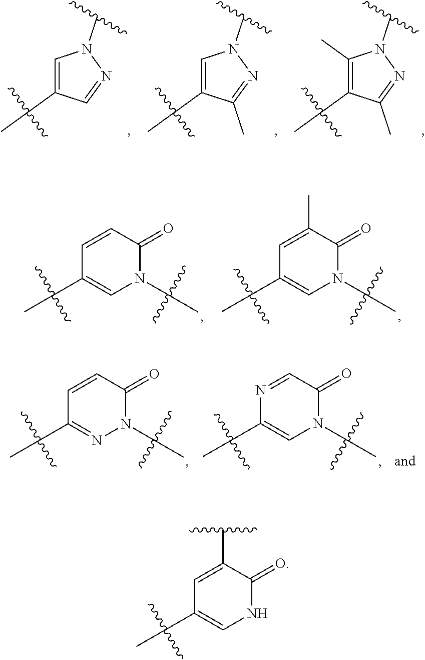

72. The compound according to any one of embodiments 1-52 and 68-71, wherein Ring D′ is a 4- to 6-membered carbocyclic ring substituted with t instances of -L-W.
73. The compound according to any one of embodiments 1-52 and 68-71, wherein Ring D′ is a 4- to 6-membered heterocyclic ring having 1-2 heteroatoms independently selected from nitrogen, oxygen, and sulfur, wherein Ring D′ is substituted with t instances of -L-W.
74. The compound according to any one of embodiments 1-52 and 68-71, wherein Ring D′, substituted with t instances of -L-W, is selected from

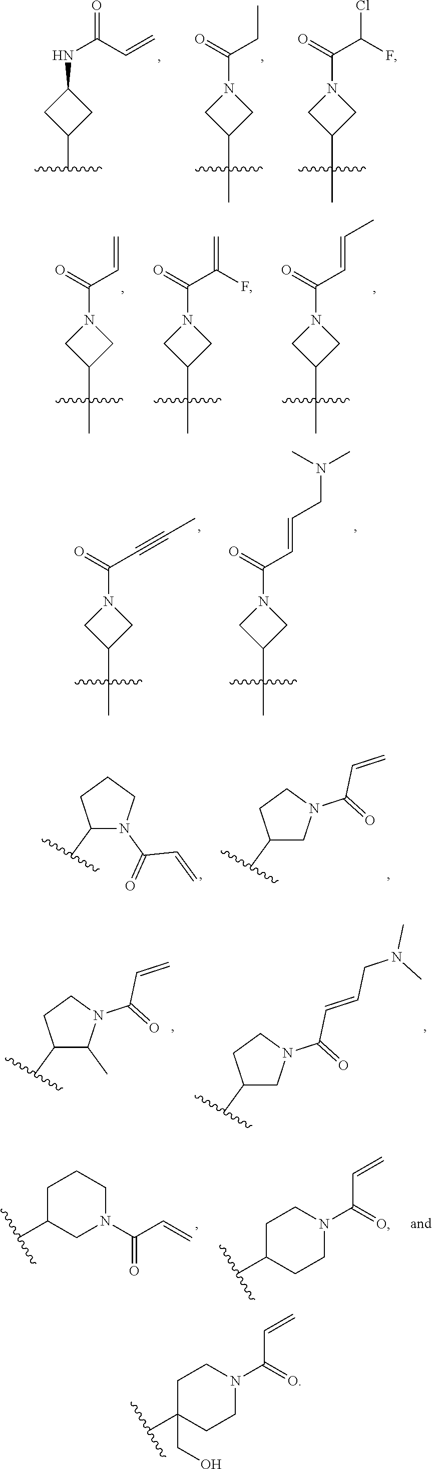

75. The compound according to embodiment 70, wherein

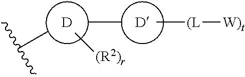

is selected from

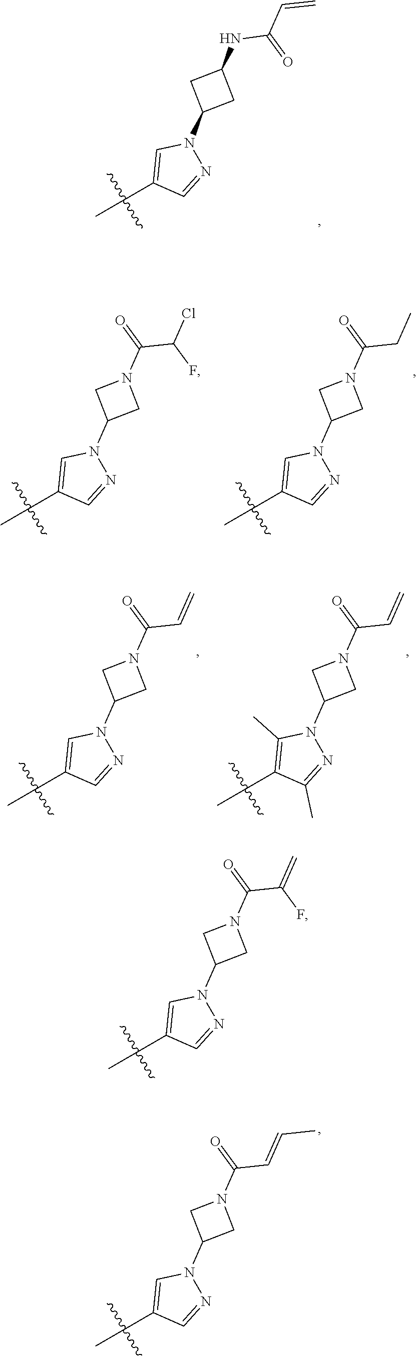

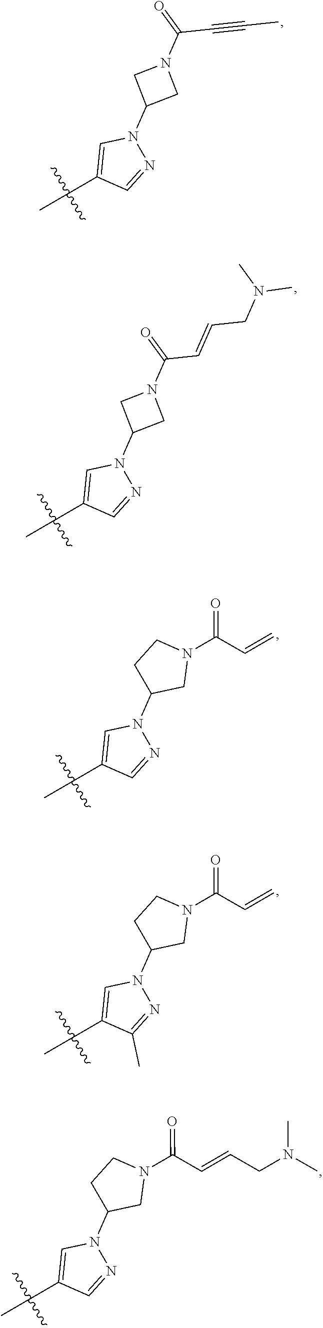 [SMILES: *c1cnn(C2CN(C(=O)C#CC)C2)c1 ; *c1cnn(C2CN(C(=O)/C=C/CN(C)C)C2)c1 ; *c1cnn(C2CCN(C(=O)C=C)C2)c1 ; *c1cn(C2CCN(C(=O)C=C)C2)nc1C ; *c1cnn(C2CCN(C(=O)/C=C/CN(C)C)C2)c1]

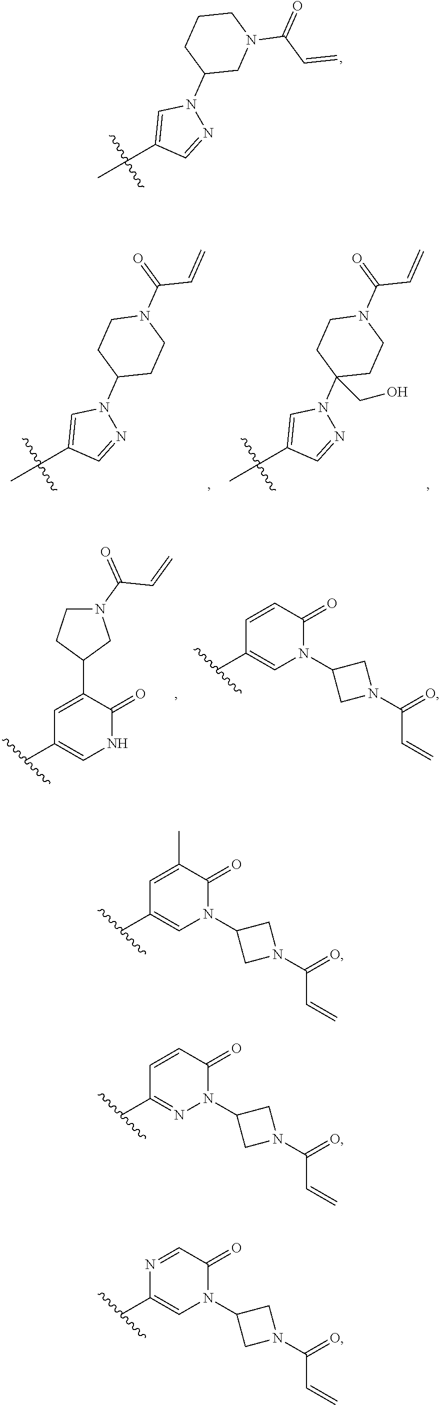

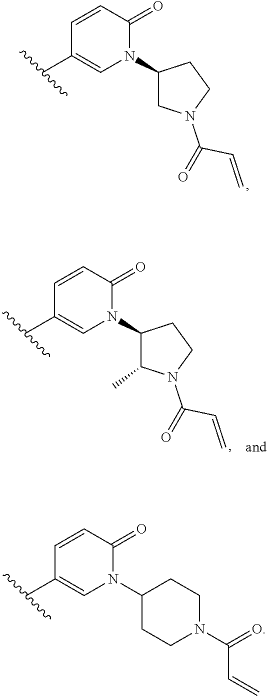 [SMILES: *c1ccc(=O)n([C@H]2CCN(C(=O)C=C)C2)c1 ; *c1ccc(=O)n([C@H]2CCN(C(=O)C=C)[C@@H]2C)c1 ; *c1ccc(=O)n(C2CCN(C(=O)C=C)CC2)c1]

76. The compound according to any one of embodiments 1-52 and 68-75, wherein the compound is selected from Formulae I-a-iv, I-b-iv, I-c-iv, J-d-iv, I-e-iv, I-f-iv, I-g-iv, and I-h-iv:

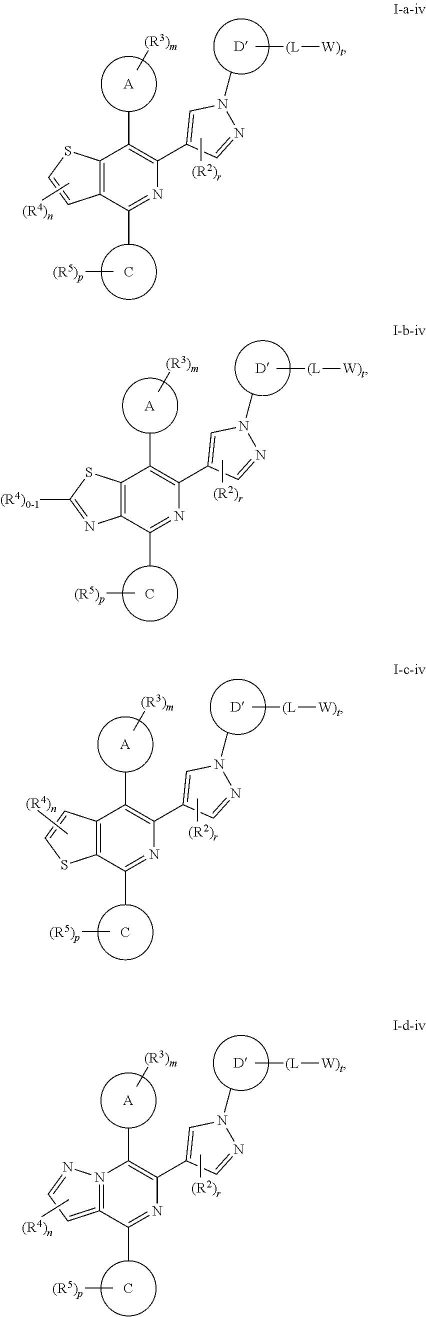

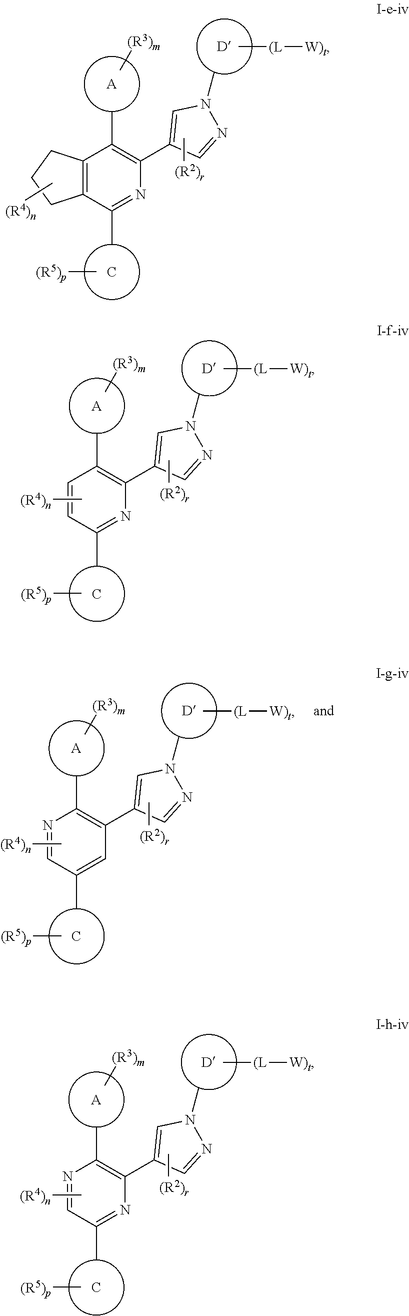

or a pharmaceutically acceptable salt thereof
77. The compound according to any one of embodiments 1-36, 38-52, and 54-67, wherein the compound is selected from Formulae I-a-v, I-b-v, I-c-v, I-d-v, I-e-v, I-f-v, I-g-v, and I-h-v:

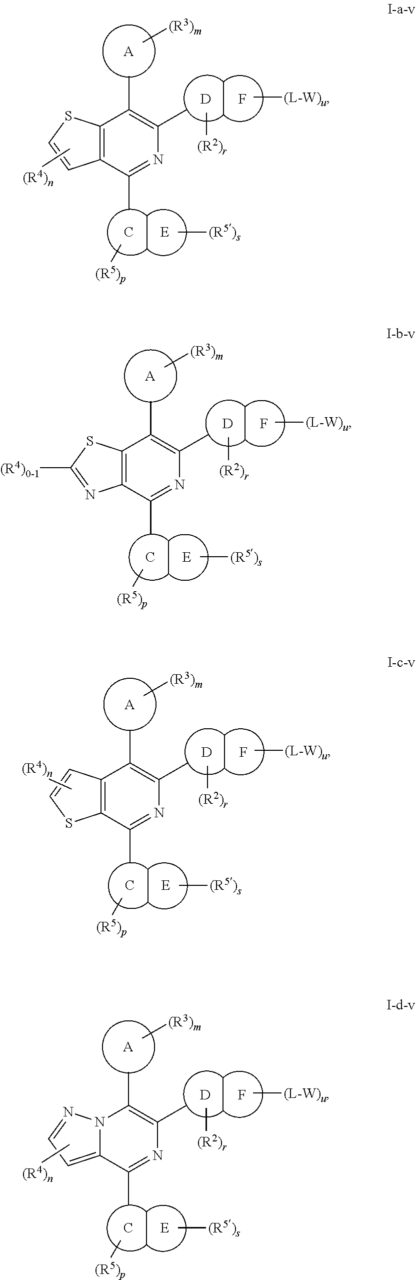

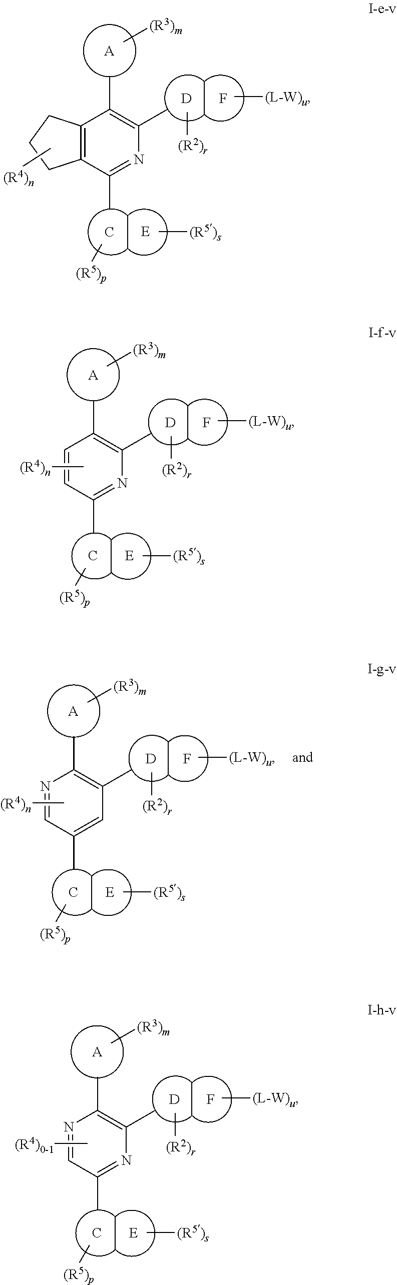

or a pharmaceutically acceptable salt thereof
78. The compound according to any one of embodiments 1-52 and 68-76, wherein the compound is selected from Formulae I-1, I-m, I-n, I-p, and I-q:

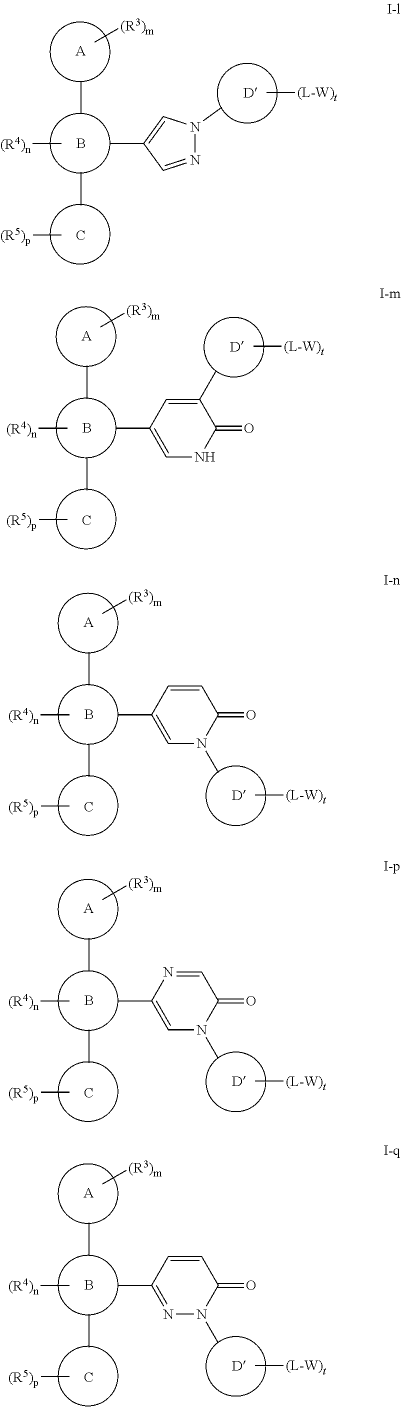

or a pharmaceutically acceptable salt thereof.
79. The compound according to any one of embodiments 1-78, wherein t is 1.
80. The compound according to any one of embodiments 1-79, wherein q is 1.
81. The compound according to any one of embodiments 1-80, wherein v is 0 or 1.
82. The compound according to any one of embodiments 1-81, wherein x is 0 or 1.
83. The compound according to any one of embodiments 1-82, wherein each R is independently hydrogen, C₁₋₆ aliphatic, or C₁₋₆ haloaliphatic.
84. A compound selected from Table 1, or a pharmaceutically acceptable salt thereof.
85. A pharmaceutical composition comprising a compound according to any one of embodiments 1-84, or a pharmaceutically acceptable salt thereof, and a pharmaceutically acceptable carrier.
86. A method comprising administering a therapeutically effective amount of a compound according to any one of embodiments 1-84 or a pharmaceutically acceptable salt thereof to a subject in need thereof.
87. The method according to embodiment 86, wherein the subject has a disease, disorder, or condition ameliorated by disruption, inhibition, and/or prevention of an interaction between a small GTPase and a PI3Kα protein.
88. The method of embodiment 87, wherein the small GTPase is Rac1, CDC42, or a RAS protein.
89. The method of embodiment 88, wherein the small GTPase is a RAS protein.
90. The method of embodiment 89, wherein the RAS protein is KRAS, NRAS, HRAS, RRAS, RRAS2, MRAS, or RIT1.
91. The method according to any one of embodiments 86-90, wherein the subject has a cancer.
92. A method of treating a cancer, comprising administering to a subject in need thereof a therapeutically effective amount of a compound according to any one of embodiments 1-84 or a pharmaceutically acceptable salt thereof.
93. The method according to embodiment 91 or 92, wherein the cancer is associated with and/or characterized by aberrant activation of PI3Kα and/or a mutation in PI3Kα.
94. The method according to embodiment 93, wherein the PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation.
95. The method according to any one of embodiments 91-94, wherein the cancer is characterized by a mutation in a RAS protein.
96. The method according to embodiment 95, wherein the RAS protein comprises a mutation in codon 12, 13, or 61.
97. The method according to embodiment 95 or 96, wherein the RAS protein is KRAS.
98. The method according to embodiment 97, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
99. The method according to embodiment 95 or 96, wherein the RAS protein is HRAS.
100. The method according to embodiment 99, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.
101. The method according to embodiment 95 or 96, wherein the RAS protein is NRAS.
102. The method according to embodiment 101, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
103. The method according to any one of embodiments 91-102, wherein the cancer is selected from pancreatic cancer; colon cancer; rectal cancer; colorectal cancer; breast cancer; ovarian cancer; endometrial cancer; lung cancer; prostate cancer; cancers of the oral cavity and pharynx (lip, tongue, mouth, larynx, pharynx), esophagus, stomach, small intestine, large intestine, liver and biliary passages, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease; non-Hodgkin's lymphomas; multiple myeloma; and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic lymphomas.
104. The method according to embodiment 103, wherein the cancer is selected from breast cancer, lung cancer (e.g., non-small cell lung cancer), endometrial cancer, esophageal cancer, ovarian cancer, colorectal cancer, gastric cancer, squamous cell carcinoma, prostate cancer, and pancreatic cancer.
105. The method according to any one of embodiments 91-104, wherein the cancer is characterized by mutated, overexpressed, and/or amplified receptor tyrosine kinases (e.g., HER family, Met, FGFR, Alk, PDGF, EGFR, or ROS kinases).
106. The method according to any one of embodiments 91-105, wherein the cancer is characterized by a mutation in or a deletion of a PTEN protein.
107. The method according to any one of embodiments 86-106, wherein the subject has previously undergone a treatment regimen for cancer.
108. The method according to any one of embodiments 86-107, wherein the subject has previously entered remission from cancer.
109. A method of treating a metabolic disorder, comprising administering to a subject in need thereof a therapeutically effective amount of a compound according to any one of embodiments 1-84 or a pharmaceutically acceptable salt thereof.
110. The method according to embodiment 109, wherein the metabolic disorder is selected from hyperinsulinemia and type 2 diabetes.
111. A method of treating a RASopathy, comprising administering to a subject in need thereof a therapeutically effective amount of a compound according to any one of embodiments 1-84 or a pharmaceutically acceptable salt thereof.
112. The method according to embodiment 111, wherein the RASopathy is selected from neurofibromatosis type 1 (NF1), capillary malformation-arteriovenous malformation syndrome, and Legius syndrome.
113. A method of treating a vascular disorder or condition, comprising administering to a subject in need thereof a therapeutically effective amount of a compound according to any one of embodiments 1-84 or a pharmaceutically acceptable salt thereof.
114. The method according to embodiment 113, wherein the vascular disorder or condition is selected from PIK3CA-related overgrowth syndrome (PROS) and vascular malformations (e.g., venous malformations; lymphatic malformations; congenital lipomatous overgrowth with vascular, epidermal, and skeletal anomalies syndrome (CLOVES); Klippel-Trenaunay Syndrome; PTEN hamartoma tumor syndrome (PHTS); and fibro-adipose vascular anomaly (FAVA)).
115. A method of disrupting an interaction between a small GTPase and a PI3Kα protein in a subject, comprising administering to the subject a compound according to any one of embodiments 1-84 or a pharmaceutically acceptable salt thereof.
116. A method of disrupting an interaction between a small GTPase and a PI3Kα protein, comprising contacting a cell containing the small GTPase and the PI3Kα protein with a compound according to any one of embodiments 1-84 or a pharmaceutically acceptable salt thereof.
117. A method comprising contacting a cell containing a small GTPase and a PI3Kα protein with a compound according to any one of embodiments 1-84 or a pharmaceutically acceptable salt thereof.
118. The method of embodiment 116 or 117, wherein the cell is included in a subject.
119. The method according to any one of embodiments 115-118, wherein the small GTPase is selected from Rac1, CDC42, and a RAS protein.
120. The method according to embodiment 119, wherein the small GTPase is a RAS protein.
121. The method according to embodiment 120, wherein the RAS protein is selected from HRAS, NRAS, KRAS, RRAS, RRAS2, MRAS, and RIT1.
122. The method according to embodiment 121, wherein the RAS protein is KRAS.
123. The method according to embodiment 122, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
124. The method according to embodiment 121, wherein the RAS protein is HRAS.
125. The method according to embodiment 124, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.
126. The method according to embodiment 121, wherein the RAS protein is NRAS.
127. The method according to embodiment 126, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
128. A compound according to any one of embodiments 1-84 or a pharmaceutically acceptable salt thereof for use as a medicament.
129. Use of a compound according to any one of embodiments 1-84 or a pharmaceutically acceptable salt thereof in the manufacture of a medicament.
130. The compound or use according to embodiment 128 or 129, wherein the medicament is for treating a cancer.
131. The compound or use according to embodiment 130, wherein the cancer is associated with and/or characterized by aberrant activation of PI3Kα and/or a mutation in PI3Kα.
132. The compound or use according to embodiment 131, wherein the PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation.
133. The compound or use according to any one of embodiments 130-132, wherein the cancer is characterized by a mutation in a RAS protein.
134. The compound or use according to embodiment 133, wherein the RAS protein comprises a mutation in codon 12, 13, or 61.
135. The compound or use according to embodiment 133 or 134, wherein the RAS protein is KRAS.
136. The compound or use according to embodiment 135, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
137. The compound or use according to embodiment 133 or 134, wherein the RAS protein is HRAS.
138. The compound according to embodiment 137, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.
139. The compound or use according to embodiment 133 or 134, wherein the RAS protein is NRAS.
140. The compound or use according to embodiment 139, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
141. The compound or use according to any one of embodiments 130-140, wherein the cancer is selected from pancreatic cancer; colon cancer; rectal cancer; colorectal cancer; breast cancer; ovarian cancer; endometrial cancer; lung cancer; prostate cancer; cancers of the oral cavity and pharynx (lip, tongue, mouth, larynx, pharynx), esophagus, stomach, small intestine, large intestine, liver and biliary passages, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease; non-Hodgkin's lymphomas; multiple myeloma; and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic lymphomas.
142. The compound or use according to embodiment 141, wherein the cancer is selected from breast cancer, lung cancer (e.g., non-small cell lung cancer), endometrial cancer, esophageal cancer, ovarian cancer, colorectal cancer, gastric cancer, squamous cell carcinoma, prostate cancer, and pancreatic cancer.
143. The compound or use according to any one of embodiments 130-142, wherein the cancer is characterized by mutated, overexpressed, and/or amplified receptor tyrosine kinases (e.g., HER family, Met, FGFR, Alk, PDGF, EGFR, or ROS kinases).
144. The compound or use according to any one of embodiments 130-143, wherein the cancer is characterized by a mutation in or a deletion of a PTEN protein.
145. The compound or use according to embodiment 128 or 129, wherein the medicament is for treating a metabolic disorder, a RASopathy, or a vascular disorder.
146. The compound or use according to embodiment 145, wherein: (i) the metabolic disorder is selected from hyperinsulinemia and type 2 diabetes; (ii) the RASopathy is selected from neurofibromatosis type 1 (NF1), capillary malformation-arteriovenous malformation syndrome, and Legius syndrome; or (iii) the vascular disorder or condition is selected from PIK3CA-related overgrowth syndrome (PROS) and vascular malformations (e.g., venous malformations; lymphatic malformations; congenital lipomatous overgrowth with vascular, epidermal, and skeletal anomalies syndrome (CLOVES); Klippel-Trenaunay Syndrome; PTEN hamartoma tumor syndrome (PHTS); or fibro-adipose vascular anomaly (FAVA)).
147. A compound according to any one of embodiments 1-84 or a pharmaceutically acceptable salt thereof for use in treating a disease, disorder, or condition.
148. The compound for use according to embodiment 147 for use in treating a cancer.
149. The compound for use according to embodiment 148, wherein the cancer is associated with and/or characterized by aberrant activation of PI3Kα and/or a mutation in PI3Kα.
150. The compound for use according to embodiment 149, wherein the PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation.
151. The compound for use according to any one of embodiments 148-150, wherein the cancer is characterized by a mutation in a RAS protein.
152. The compound for use according to embodiment 151, wherein the RAS protein comprises a mutation in codon 12, 13, or 61.
153. The compound for use according to embodiment 151 or 152, wherein the RAS protein is KRAS.
154. The compound for use according to embodiment 153, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
155. The compound for use according to embodiment 151 or 152, wherein the RAS protein is HRAS.
156. The compound for use according to embodiment 155, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.
157. The compound for use according to embodiment 151 or 152, wherein the RAS protein is NRAS.
158. The compound for use according to embodiment 157, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
159. The compound for use according to any one of embodiments 148-158, wherein the cancer is selected from pancreatic cancer; colon cancer; rectal cancer; colorectal cancer; breast cancer; ovarian cancer; endometrial cancer; lung cancer; prostate cancer; cancers of the oral cavity and pharynx (lip, tongue, mouth, larynx, pharynx), esophagus, stomach, small intestine, large intestine, liver and biliary passages, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease; non-Hodgkin's lymphomas; multiple myeloma; and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic lymphomas.
160. The compound for use according to embodiment 159, wherein the cancer is selected from breast cancer, lung cancer (e.g., non-small cell lung cancer), endometrial cancer, esophageal cancer, ovarian cancer, colorectal cancer, gastric cancer, squamous cell carcinoma, prostate cancer, and pancreatic cancer.
161. The compound for use according to any one of embodiments 148-160, wherein the cancer is characterized by mutated, overexpressed, and/or amplified receptor tyrosine kinases (e.g., HER family, Met, FGFR, Alk, PDGF, EGFR, or ROS kinases).
162. The compound for use according to any one of embodiments 148-161, wherein the cancer is characterized by a mutation in or a deletion of a PTEN protein.
163. The compound for use according to embodiment 147 for use in treating a metabolic disorder, a RASopathy, or a vascular disorder.
164. The compound for use according to embodiment 163, wherein: (i) the metabolic disorder is selected from hyperinsulinemia and type 2 diabetes; (ii) the RASopathy is selected from neurofibromatosis type 1 (NF1), capillary malformation-arteriovenous malformation syndrome, and Legius syndrome; or (iii) the vascular disorder or condition is selected from PIK3CA-related overgrowth syndrome (PROS) and vascular malformations (e.g., venous malformations; lymphatic malformations; congenital lipomatous overgrowth with vascular, epidermal, and skeletal anomalies syndrome (CLOVES); Klippel-Trenaunay Syndrome; PTEN hamartoma tumor syndrome (PHTS); and fibro-adipose vascular anomaly (FAVA)).
165. A compound capable of disrupting an interaction between a small GTPase and a PI3Kα protein.
166. The compound according to embodiment 165, wherein the compound is capable of binding to PI3Kα, such that (i) the interaction between the small GTPase and PI3Kα is at least partially disrupted, prevented, or inhibited; and/or (ii) the kinase activity of PI3Kα is not significantly inhibited.
167. The compound according to embodiment 165 or 166, wherein the compound has an activity of:
   (i) <5 μM or ≥5 μM and ≤25 μM in the assay of Biological Example 1 (e.g., a surface plasmon resonance (SPR) binding assay assessing inhibition of the KRAS-PI3Kα interaction);
   (ii) ≥75%, <75% and ≥50%, or ≤50% and ≥25% in the assay of Biological Example 2 (e.g., a Matrix-Assisted Laser Desorption Ionization-Time of Flight Mass Spectrometry (MALDI-TOF MS) analysis of covalent modification of Cys242 in human PIK3CA (157-299);
   (iii) <1 μM or ≥1 μM and ≤10 μM in the assay of Biological Example 3 (e.g., a matrix-assisted cell-based pAKT homogenous time-resolved fluorescence (HTRF) assay in Tet-on KRAS G12D HeLa cells); and/or
   (iv) <0.1 μM, ≥0.1 μM and ≤1 μM, or ≥1 μM and ≤3 μM in the assay of Biological Example 4 (e.g., a matrix-assisted cell-based pAKT HTRF assay in BT474 cells).
168. The compound according to any one of embodiments 165-167, wherein the compound comprises an electrophilic moiety.
169. The compound according to any one of embodiments 165-168, wherein the compound is capable of interacting with a Cys242 residue in the catalytic subunit of PI3Kα.
170. The compound according to any one of embodiments 165-169, wherein the compound is capable of irreversibly binding the PI3Kα protein.
171. The compound according to any one of embodiments 165-169, wherein the compound is capable of reversibly binding the PI3Kα protein.
172. The compound according to any one of embodiments 165-171, wherein the small GTPase is Rac1, CDC42, or a RAS protein.
173. The compound according to embodiment 172, wherein the RAS protein is KRAS, NRAS, HRAS, RRAS, RRAS2, MRAS, or RIT1.
174. The compound according to embodiment 173, wherein the RAS protein comprises a mutation in codon 12, 13, or 61.
175. The compound according to embodiment 173 or 174, wherein the RAS protein is KRAS.
176. The compound according to embodiment 175, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
177. The compound according to embodiment 173 or 174, wherein the RAS protein is HRAS.
178. The compound according to embodiment 177, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.
179. The compound according to embodiment 173 or 174, wherein the RAS protein is NRAS.
180. The compound according to embodiment 179, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
181. The compound of any one of embodiments 165-180, wherein the compound is a compound according to any one of embodiments 1-84.
182. A method of disrupting, inhibiting, or preventing an interaction between a small GTPase and a PI3Kα protein, comprising contacting a cell containing the small GTPase and the PI3Kα protein with a compound according to any one of embodiments 165-181 or a pharmaceutically acceptable salt thereof.
183. The method according to embodiment 182, wherein the cell is within a subject.
184. The method according to embodiment 182 or 183, wherein the small GTPase is Rac1, CDC42, or a RAS protein.
185. The method according to embodiment 184, wherein the RAS protein is KRAS, NRAS, HRAS, RRAS, RRAS2, MRAS, or RIT1.
186. The method according to embodiment 185, wherein the RAS protein comprises a mutation in codon 12, 13, or 61.
187. The method according to embodiment 185 or 186, wherein the RAS protein is KRAS.
188. The method according to embodiment 187, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
189. The method according to embodiment 185 or 186, wherein the RAS protein is HRAS.
190. The method according to embodiment 189, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.
191. The method according to embodiment 185 or 186, wherein the RAS protein is NRAS.
192. The method according to embodiment 191, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

Selected abbreviations used in the preceding sections and the Examples are summarized in Table 3.

TABLE 3
Abbreviations.

| Abbreviation | Term |
|---|---|
| MeCN, ACN | acetonitrile |
| cm | centimeter |
| ° C. | degrees Celsius |
| ° K | degrees Kelvin |
| BINAP | [1,1′-Binaphthalene]-2,2′-diyl)bis(diphenylphosphane) |
| DCM | dichloromethane |
| DIEA | N,N-diisopropylethylamine (Húnig's base) |
| DMF | dimethylformamide |
| DMSO | dimethyl sulfoxide |
| ESI | electrospray ion-mass spectrometry |
| EtOH | ethanol |
| EtOAc, EA | ethyl acetate |
| g | gram |
| Hz | hertz |
| HATU | Hexafluorophosphate azabenzotriazole tetramethyl uronium |
| HPLC | high performance liquid chromatography |
| HTRF | homogenous time-resolved fluorescence |
| h | hour |
| kDa | kilodalton |
| LC | liquid chromatography |
| LCMS | liquid chromatography-mass spectrometry |
| L | liter |
| MS | mass spectra |
| MHz | megahertz |
| MeOH | methanol |
| MTBE | methyl tert-butyl ether |
| μg | microgram |
| μL | microliter |
| μM | micromolar |
| μm | micron |
| μs | microsecond |
| mg | milligram |
| mL | milliliter |
| mm | millimeter |
| mM | millimolar |
| mmol | millimole |
| min | minute |
| M | molar |
| nL | nanoliter |
| nm | nanometer |
| NCS | N-chlorosuccinimide |
| NMP | N-methyl-2-pyrrolidone |
| PE:EA | Petroleum ether:ethyl acetate |
| ppm | parts per million |
| PTLC | preparative thin layer chromatography |
| ¹HNMR | proton nuclear magnetic resonance |
| RBD | receptor binding domain |
| RP | reverse phase |
| rpm | revolutions per minute |
| SFC | supercritical fluid chromatography |
| SPR | surface plasmon resonance |
| THF | tetrahydrofuran |
| TMS | tetramethylsilane |
| TLC | thin layer chromatography |
| SOCl₂ | thionyl chloride |
| TEA | triethylamine |
| TFA | trifluoroacetic acid |
| TCEP | tris(2-carboxyethyl)phosphine |
| UV | ultraviolet |
| UV/Vis | ultraviolet/visible |

### Materials and Methods

Preparative thin layer chromatography (PTLC) separations described herein were typically performed on 20×20 cm plates (500-μm thick silica gel).

Chromatographic purifications were typically performed using Biotage Isolera. One automated system running Biotage Isolera One 2.0.6 software (Biotage LLC, Charlotte, NC). Flow rates were the default values specified for the column in use. Reverse phase chromatography was performed using elution gradients of water and acetonitrile on KP-C18-HS Flash+columns (Biotage LLC) of various sizes. Typical loading was between 1:50 and 1:1000 crude sample: RP SiO₂ by weight. Normal phase chromatography was performed using elution gradients of various solvents (e.g., hexane, ethyl acetate, methylene chloride, methanol, acetone, chloroform, MTBE, etc.). The columns were SNAP Cartridges containing KP-SIL or SNAP Ultra (25 pm spherical particles) of various sizes (Biotage LLC). Typical loading was between 1:10 to 1:150 crude sample: SiO₂ by weight. Alternatively, silica gel chromatography was performed on a Biotage Horizon flash chromatography system.

¹HNMR analyses of intermediates and exemplified compounds were typically performed on a Bruker Ascend TM 400 spectrometer (operating at 400 MHz), Bruker Ascend 500 MHz Avance Neo Spectrometer (Bruker-Biospin) or Bruker Avance Neo Nanobay (operating at 400 MHz) at 298° K following standard operating procedure suggested by manufacturer. Reference frequency was set using TMS as an internal standard. Chemical shift values (6) are reported in parts per million (ppm) with splitting pattems abbreviated to: s (singlet), br. s (broad singlet), d (doublet), dd (double doublet), t (triplet), and m (multiplet). The coupling constant (J) is given in Hz. Typical deuterated solvents were utilized as indicated in the individual examples.

LCMS analysis was typically performed using one of the following conditions:

(1) LCMS spectra were taken on an Agilent Technologies 6120B Quadrupole spectrometer. The mobile phase for the LC was acetonitrile (A) with 0.1% formic acid, and water (B) with 0.1% formic acid, and the eluent gradient was from 5-95% A in 6.0 min, 5%-40% A in 6.0 min, 80-100% A in 6.0 min. using a poroshell 120 EC-C18 50 mm×3.0 mm×2.7 μM capillary column; Flow Rate: 0.7 mL/min. Mass spectra (MS) were measured by electrospray ion-mass spectroscopy (ESI). All temperatures are in degrees Celsius (° C.) unless otherwise noted.

(2) LCMS spectra were taken on an Agilent Technologies 1290-6420 Triple Quadrupole spectrometer: The mobile phase for the LC was acetonitrile (A) with 0.05% formic acid, and water (B) with 0.05% formic acid, and the eluent gradient was from 5-95% A in 5.0 min, using a ZORBAX SB—C18 50 mm×2.1 mm×1.8 μM capillary column; Flow Rate: 0.3 mL/min. Mass spectra (MS) were measured by electrospray ion-mass spectroscopy (ESI). All temperatures are in degrees Celsius unless otherwise noted.

(3) LC-MS analysis was performed using an Agilent 6120b single quadrupole mass spectrometer with an Agilent 1260 infinity II chromatography separations module and Agilent 1260 infinity II photodiode array detector controlled by Agilent Chemstation software. The HPLC column used was an Agilent ZORBAX Eclipse XDB-C18 4.6 mm×150 mm×3.5 μM RapidResol column with a mobile phase of water (0.1% formic acid)/MeCN (0.1% formic acid) and a gradient of 5-95% MeCN over 10 minutes at a flow rate of 1 mL/min. Accurate mass data was obtained using a Thermo Fisher extractive plus EMR orbitrap LCMS system. Exact mass values were calculated by ChemCalc.

(4) LCMS spectra were taken on an alliance Waters 2695 coupled to a Waters 2487 Dual Wavelength Absorbance Detector and a Waters Micromass-ZQ-2000 single quadrupole spectrometer. The mobile phase for the LC was acetonitrile (A) and water (B) with 0.01% formic acid, and the eluent gradient was from 5-100% A in 10.0 minute using a Kromasil 100-5-C18 150 mm×4.6 mm×5 μm column. Mass spectra (MS) were measured by electrospray ion-mass spectroscopy (ESI). All temperatures are in degrees Celsius unless otherwise noted.

(5) LCMS spectra were taken on Waters Micromass-ZQ 2000 Quadrupole spectrometer. The mobile phase for the LC was (A) 0.1% formic acid in water; (B) Acetonitrile 100% and the eluent gradient was from 10-90% B in 10.0 min, 90% up to 12 min B, 12-13 min 90-10% B, 13-15 min 90-10% B using Phenomenex Gemini-C18 (50 mm×4.6 mm×5 μm); Flow Rate: 0.5 mL/min. Mass spectra (MS) were measured by Electrospray Ion-Mass spectroscopy (ESI).

Typically, analytical HPLC spectrometry conditions were as follows:

LC1: Agilent Technologies 1260 Infinity coupled, Column: poroshell 120 EC-C18 150 mm×4.6 mm×4 m; Temperature: 40° C.; Eluent: 5:95 v/v acetonitrile/water+0.02% trifluoroacetic acid in 20 min; Flow Rate: 1.2 mL/min; Detection: VWD, 190-600 nm.

LC2: Shimadzu 2010 CHT, Column Waters X-select CSH C18 (150×4.6) mm×3.5 μm, Temperature: 30° C.; MP-A 10 mm ammonium acetate Buffer, MP-B: Acetonitrile (100%), Flow Rate: 1.0 mL/min; Detection: VWD, 270 nm. Gradient elevation: time/B con: 0/5, 2/5, 20/50, 25/50, 30/90, 35/90, 37/05, 40/05.

LC3: Shimadzu LC-2010A HT, Column: XBRIDGE-C18 3.5 m 2.1×50 mm; Temperature: 45° C.; Mobile phase: water (0.05% TFA)-ACN (0.05% TFA), ACN from 0 to 60% over 7 minutes, 7-8 min, ACN from 60% to 100%; Flow Rate: 0.8 mL/min; Detection: PDA, 214 nm, 254 nm.

LC4: Shimadzu LC-2050c, Column: XBRIDGE-C18 3.5 μm 2.1×50 mm; Temperature: 45° C.; Mobile phase: water (0.05% TFA)-ACN (0.05% TFA), ACN from 0 to 60% over 7 minutes, 7-8 min, ACN from 60% to 100%; Flow Rate: 0.8 mL/min; Detection: VWD, PDA, 214 nm, 254 nm. PDA, 214 nm, 254 nm.

Preparative HPLC was carried out with one of the following conditions:

Condition 1: GILSON Preparative HPLC System; Column: Ultimate XB—C18, 21.2 mm×250 mm, 5 m; Mobile phase: Water with 0.1% trifluoroacetic acid; MeCN with 0.1% trifluoroacetic acid; Method: 15 minutes gradient elution; Initial organic: 10% to 30%; Final organic: 60% to 80%; UV1: 240; UV2: 230; Flow: 15 mL/min.

Condition 2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 uM OBD™ 19×250 mm column with a mobile phase of water/MeCN or water (0.1% TFA)/MeCN (0.1% TFA).

Condition 3: Shimadzu Preparative HPLC System; Column: Phenomenex Luna C18, 21.1 mm×250 mm, 10 m; Mobile phase; MP-A 10 mm ammonium acetate Buffer, MP-B: Methanol (100%), 35 minutes gradient elution UV: 254; Flow: 10 mL/min. Gradient elevation: time/B con: 0/50, 25/90, 30/90, 32/50, 35/50.

Chiral supercritical fluid chromatography (SFC) was carried out with one of the following conditions:

Condition 1: SFC Thar prep 80; Column: CHIRALPAK® AD-H 250 mm×20 mm, 5 m; Mobile phase: 40% EtOH/CO₂ (containing 0.2% NH₄OH); 40 g/min.

Condition 2: SFC Thar prep 80; Column: CHIRALPAK@OD-H 250 mm×20 mm, 5 μm; Mobile phase: 40% MeOH/CO₂ (containing 0.2% NH₄OH); 40 g/min.

Condition 3: SFC Thar prep 80; Column: CHIRALPAK® IC 250 mm×20 mm, 5 m; Mobile phase: 40% IPA/CO₂ (containing 0.2% DEA); 40 g/min.

Compound names were generated with ChemDraw Professional.

The compounds provided herein, including in various forms such as salts, esters, tautomers, prodrugs, zwitterionic forms, stereoisomers, etc., may be prepared according to various methods including those set forth in the following examples.

### Synthesis of Provided Compounds

### Synthetic Example 1: Synthesis of N-(3-(4-(2-(2-methoxvethoxy)phenyl)-1-(1,2,3,4-tetrahydroisoquinolin-6-yl)-6,7-dihydro-5H-cyclopenta[c]pyridin-3-yl)benzyl)acrylamide (Compound 1)

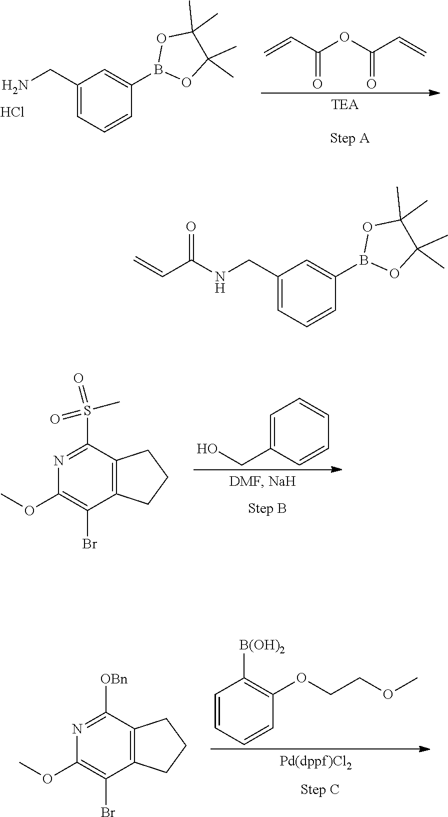

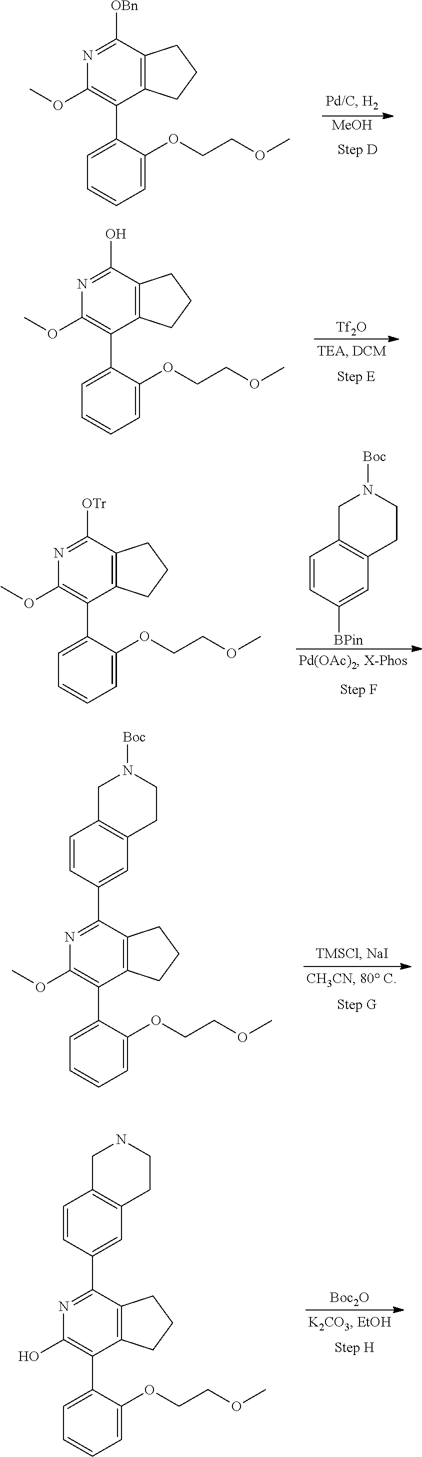

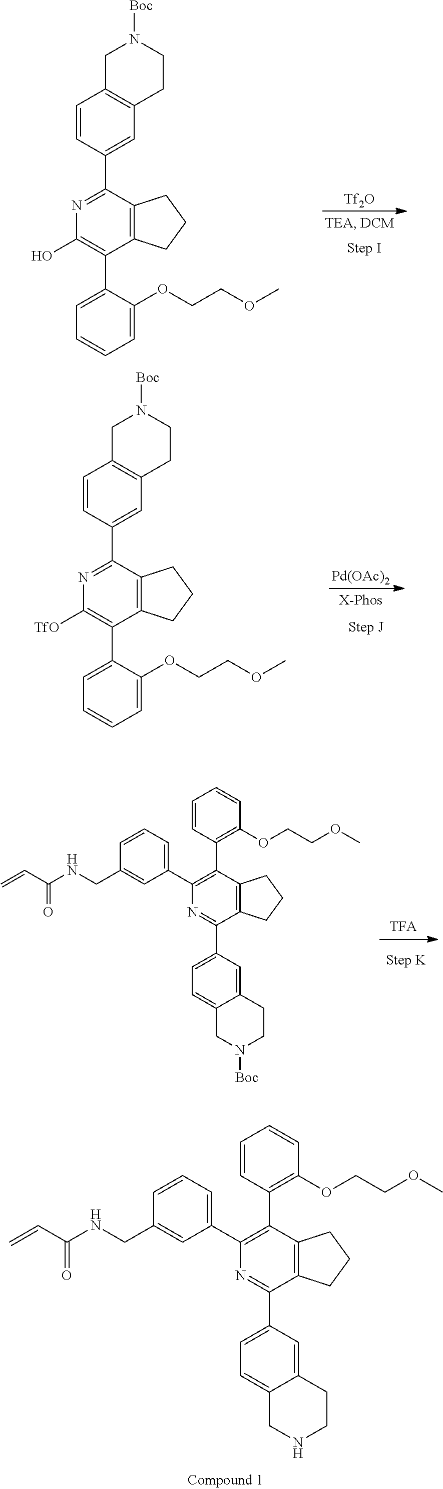

Step A: Preparation of N-(3-(4,4,5,5-tetramethyl-1,3,2-dioxaborolan-2-yl)benzyl)acrylamide: To a stirred solution of [3-(4,4,5,5-tetramethyl-1,3,2-dioxaborolan-2-yl)phenyl]methanamine hydrochloride (100 mg, 0.371 mmol) and triethylamine (0.16 mL, 1.1 mmol) in DCM (5 mL) was added acrylic anhydride (70 mg, 0.56 mmol). The solution was stirred at 25° C. for 1 h. The mixture was concentrated and purified by preparative-TLC (petroleum ether/EtOAc: 3/1) to give N-(3-(4,4,5,5-tetramethyl-1,3,2-dioxaborolan-2-yl)benzyl)acrylamide (100 mg, 94% yield) as a colorless oil. LCMS ESI (+) m/z 288.2 (M+H).

Step B: Preparation of 1-benzyloxy-4-bromo-3-methoxy-6,7-dihydro-5H-cyclopenta[c]pyridine: To a solution of benzyl alcohol (3.80 mL, 36.3 mmol) stirred in DMF (50 mL) at 0° C. was added sodium hydride (0.870 g, 36.3 mmol) by portions. The mixture was stirred at 0° C. for 30 min. 4-Bromo-3-methoxy-1-methylsulfonyl-6,7-dihydro-5H-cyclopenta[c]pyridine (3.70 g, 12.1 mmol) was added, and the resulting mixture was stirred at 0° C. to 25° C. for additional 4 hours. The mixture was diluted with water and extracted with EtOAc. The organic layers were dried over Na₂SO₄ and concentrated. The residue was purified by silica gel column (EtOAc/petroleum ether: 0 to 10%) to give 1-benzyloxy-4-bromo-3-methoxy-6,7-dihydro-5H-cyclopenta[c]pyridine (3.90 g, 97% yield). LCMS ESI (+) m/z 334.0 (M+H).

Step C: Preparation of 1-(benzyloxy)-3-methoxy-4-(2-(2-methoxyethoxy)phenyl)-6,7-dihydro-5H-cyclopenta[c]pyridine: A mixture of 1-benzyloxy-4-bromo-3-methoxy-6,7-dihydro-5H-cyclopenta[c]pyridine (1.00 g, 2.99 mmol), [2-(2-methoxyethoxy)phenyl]boronic acid (0.65 g, 3.29 mmol), 1,1′-bis(diphenylphosphino)ferrocenedichloro palladium (II) (219 mg, 0.299 mmol) and sodium carbonate (634 mg, 5.98 mmol) in 1,4-dioxane (30 mL)/water (3 mL) was stirred at 85° C. for 6 hours. The mixture was concentrated and purified by silica gel column (petroleum ether:EtOAc=10:1) to give 1-benzyloxy-3-methoxy-4-[2-(2-methoxyethoxy)phenyl]-6,7-dihydro-5H-cyclopenta[c]pyridine (1.10 g, 91% yield). LCMS ESI (+) m/z 406.2 (M+H).

Step D: Preparation of 3-methoxy-4-[2-(2-methoxyethoxy)phenyl]-6,7-dihydro-5H-cyclopenta [c]pyridin-1-ol: A mixture of 1-benzyloxy-3-methoxy-4-[2-(2-methoxyethoxy)phenyl]-6,7-dihydro-5H-cyclopenta[c]pyridine (1.10 g, 2.71 mmol) and Pd/C (100 mg) in methanol (10 mL)/THF (10 mL) was stirred at 12° C. for 12 hours. The mixture was filtered and concentrated. The residue was purified by silica gel column (petroleum ether:EtOAc=1:1) to give 3-methoxy-4-[2-(2-methoxyethoxy)phenyl]-6,7-dihydro-5H-cyclopenta [c]pyridin-1-ol (870 mg, 100% yield). LCMS ESI (+) m/z 316.2 (M+H).

Step E: Preparation of [3-methoxy-4-[2-(2-methoxyethoxy)phenyl]-6,7-dihydro-5H-cyclopenta[c]pyridin-1-yl]trifluoromethanesulfonate: To a stirred solution of 3-methoxy-4-[2-(2-methoxyethoxy)phenyl]-6,7-dihydro-5H-cyclopenta[c]pyridin-1-ol (870 mg, 2.76 mmol) and N,N-diisopropylethylamine (1.40 mL, 8.28 mmol) in DCM (10 mL) was added trifluoromethanesulfonic anhydride (0.560 mL, 3.31 mmol) at 0° C. The mixture was stirred at 10° C. for 1 hour. The mixture was diluted with EtOAc and washed with brine, dried over Na₂SO₄, filtered, concentrated and purified by silica gel column (petroleum ether:EtOAc=20:1 to 10:1) to give [3-methoxy-4-[2-(2-methoxyethoxy)phenyl]-6,7-dihydro-5H-cyclopenta[c]pyridin-1-yl]trifluoromethanesulfonate (690 mg, 56% yield). LCMS ESI (+) m/z 448.2 (M+H).

Step F: Preparation of tert-butyl 6-[3-methoxy-4-[2-(2-methoxyethoxy)phenyl]-6,7-dihydro-5H-cyclopenta[c]pyridin-1-yl]-3,4-dihydro-1H-isoquinoline-2-carboxylate: To a stirred solution of [3-methoxy-4-[2-(2-methoxyethoxy)phenyl]-6,7-dihydro-5H-cyclopenta[c]pyridin-1-yl]trifluoromethanesulfonate (300 mg, 0.670 mmol), tert-butyl 6-(4,4,5,5-tetramethyl-1,3,2-dioxaborolan-2-yl)-3,4-dihydro-1H-isoquinoline-2-carboxylate (289 mg, 0.805 mmol) and potassium carbonate (185 mg, 1.34 mmol) in MeCN (10 mL)/Water (1.2 mL) was added palladium (II) acetate (15 mg, 0.067 mmol) and X-PHOS (64 mg, 0.13 mmol). The mixture was stirred at 100° C. for 12 hours. The mixture was poured into water and extracted with EtOAc. The combined organic layers were washed with brine, dried and concentrated. The residue was purified by silica gel column (petroleum ether:EtOAc=10:1 to 4:1) to give tert-butyl 6-[3-methoxy-4-[2-(2-methoxyethoxy)phenyl]-6,7-dihydro-5H-cyclopenta[c]pyridin-1-yl]-3,4-dihydro-1H-isoquinoline-2-carboxylate (312 mg, 88% yield). LCMS ESI (+) m/z 531.4 (M+H).

Step G: Preparation of 4-(2-(2-methoxyethoxy)phenyl)-1-(1,2,3,4-tetrahydroisoquinolin-6-yl)-6,7-dihydro-5H-cyclopenta[c]pyridin-3-ol: To a stirred solution of tert-butyl 6-[3-methoxy-4-[2-(2-methoxyethoxy)phenyl]-6,7-dihydro-5H-cyclopenta[c]pyridin-1-yl]-3,4-dihydro-1H-isoquinoline-2-carboxylate (280 mg, 0.528 mmol) in MeCN (4 mL) was added sodium iodide (237 mg, 1.58 mmol) and trimethylchlorosilane (0.67 mL, 5.3 mmol). The mixture was stirred at 80° C. for 2 hours. The mixture was cooled to ambient temperature and quenched with water. The product was extracted with DCM, dried over Na₂SO₄ and concentrated to dryness to give crude product 4-(2-(2-methoxyethoxy)phenyl)-1-(1,2,3,4-tetrahydroisoquinolin-6-yl)-6,7-dihydro-5H-cyclopenta[c]pyridin-3-ol (208 mg) which was used in next step directly. LCMS ESI (+) m/z 417.2 (M+H).

Step H: Preparation of tert-butyl 6-[3-hydroxy-4-[2-(2-methoxyethoxy) phenyl]-6,7-dihydro-5H-cyclopenta[c]pyridin-1-yl]-3,4-dihydro-1H-isoquinoline-2-carboxylate: To a solution of 4-(2-(2-methoxyethoxy)phenyl)-1-(1,2,3,4-tetrahydroisoquinolin-6-yl)-6,7-dihydro-5H-cyclopenta[c]pyridin-3-ol (208 mg, 0.600 mmol) and potassium carbonate (219 mg, 1.58 mmol) in ethanol (4 mL) and H₂O (2 mL) was added di-tert-butyl dicarbonate (0.25 mL, 1.1 mmol). The mixture was stirred at ambient temperature for 2 hours. The mixture was diluted with water and the product was extracted with EtOAc. The combined organic layers were washed with brine, dried over Na₂SO₄, filtered, concentrated and purified by silica gel column (EtOAc) to give tert-butyl 6-[3-hydroxy-4-[2-(2-methoxyethoxy)phenyl]-6,7-dihydro-5H-cyclopenta[c]pyridin-1-yl]-3,4-dihydro-1H-isoquinoline-2-carboxylate (280 mg, 90%). LCMS ESI (+) m/z 517.3.

Step I: Preparation of tert-butyl 6-[4-[2-(2-methoxyethoxy)phenyl]-3-(trifluoromethylsulfonyloxy)-6,7-dihydro-5H-cyclopenta[c]pyridin-1-yl]-3,4-dihydro-1H-isoquinoline-2-carboxylate: To a solution of tert-butyl 6-[3-hydroxy-4-[2-(2-methoxyethoxy)phenyl]-6,7-dihydro-5H-cyclopenta[c]pyridin-1-yl]-3,4-dihydro-1H-isoquinoline-2-carboxylate (260 mg, 0.503 mmol) and N,N-diisopropylethylamine (0.260 mL, 1.51 mmol) in DCM (5 mL) was added trifluoromethanesulfonic anhydride (0.100 mL, 0.604 mmol). The mixture was stirred at 7° C. for 1 hour. The mixture was poured into NaHCO₃ aqueous solution. The product was extracted with EtOAc, washed with brine, dried over Na₂SO₄ and concentrated. The residue was purified by silica gel column (petroleum ether:EtOAc=4:1) to give tert-butyl 6-[4-[2-(2-methoxyethoxy)phenyl]-3-(trifluoromethylsulfonyloxy)-6,7-dihydro-5H-cyclopenta[c]pyridin-1-yl]-3,4-dihydro-1H-isoquinoline-2-carboxylate (180 mg, 55% yield). LCMS ESI (+) m/z 649.3 (M+H).

Step J: Preparation of tert-butyl 6-[4-[2-(2-methoxyethoxy)phenyl]-3-[3-[(prop-2-enoylamino)methyl]phenyl]-6,7-dihydro-5H-cyclopenta[c]pyridin-1-yl]-3,4-dihydro-1H-isoquinoline-2-carboxylate: To a solution of tert-butyl 6-[4-[2-(2-methoxyethoxy)phenyl]-3-(trifluoromethylsulfonyloxy)-6,7-dihydro-5H-cyclopenta[c]pyridin-1-yl]-3,4-dihydro-1H-isoquinoline-2-carboxylate (40 mg, 0.062 mmol) and N-[[3-(4,4,5,5-tetramethyl-1,3,2-dioxaborolan-2-yl)phenyl]methyl]prop-2-enamide (35 mg, 0.12 mmol) in acetonitrile (2 mL) and water (0.2 mL) was added X-Phos (5.9 mg, 0.012 mmol), K₂CO₃ (17 mg, 0.12 mmol) and palladium (II) acetate (1.4 mg, 0.0062 mmol). The solution was stirred for 2 hours at 100° C. under N₂. The mixture was concentrated and purified by preparative-TLC (petroleum ether:EtOAc=1:1) to give tert-butyl 6-[4-[2-(2-methoxyethoxy)phenyl]-3-[3-[(prop-2-enoylamino)methyl]phenyl]-6,7-dihydro-5H-cyclopenta[c]pyridin-1-yl]-3,4-dihydro-1H-isoquinoline-2-carboxylate (30 mg, 70% yield) as a light yellow oil. LCMS ESI (+) m/z 660.2 (M+H).

Step K: Preparation of N-[[3-[4-[2-(2-methoxyethoxy)phenyl]-1-(1,2,3,4-tetrahydroisoquinolin-6-yl)-6,7-dihydro-5H-cyclopenta[c]pyridin-3-yl]phenyl]methyl]prop-2-enamide: To a solution of tert-butyl 6-[4-[2-(2-methoxyethoxy)phenyl]-3-[3-[(prop-2-enoylamino)methyl]phenyl]-6,7-dihydro-5H-cyclopenta[c]pyridin-1-yl]-3,4-dihydro-1H-isoquinoline-2-carboxylate (30 mg, 0.043 mmol) in DCM (2 mL) was added trifluoroacetic acid (1.0 mL, 13 mmol). The solution was stirred for 1 h at ambient temperature. The solution was concentrated and purified by reverse phase preparative-HPLC to give N-[[3-[4-[2-(2-methoxyethoxy)phenyl]-1-(1,2,3,4-tetrahydroisoquinolin-6-yl)-6,7-dihydro-5H-cyclopenta[c]pyridin-3-yl]phenyl]methyl]prop-2-enamide (11 mg, 044% yield) as a trifluoroacetic acid salt. LCMS ESI (+) m/z 560.2 (M+H). ¹H NMR (400 MHz, CD₃OD) δ:7.74-7.76 (m, 2H), 7.48-7.50 (d, J=8.4 Hz, 1H), 7.40 (s, 1H), 7.26-7.35 (m, 4H), 7.02-7.04 (d, J=8.4 Hz, 1H), 6.96-6.98 (d, J=7.6 Hz, 1H), 6.86-6.90 (t, J=7.2 Hz, 1H), 6.24-6.25 (d, J=7.2 Hz, 2H), 5.69-5.72 (t, J=5.2 Hz, 1H), 4.50 (s, 2H), 4.33-4.61 (m, 2H), 4.12-4.15 (m, 1H), 3.95-3.98 (m, 1H), 3.57-3.61 (m, 4H), 3.06-3.28 (m, 8H), 2.85-2.93 (m, 1H), 2.18-2.24 (m, 2H).

### Synthetic Example 2: Synthesis of 7-[2-(2-methoxyethoxy)phenyl]-6-phenyl-4-(1,2,3,4-tetrahydroisoquinolin-6-yl)thieno[3,2-c]pyridine (Compound 4)

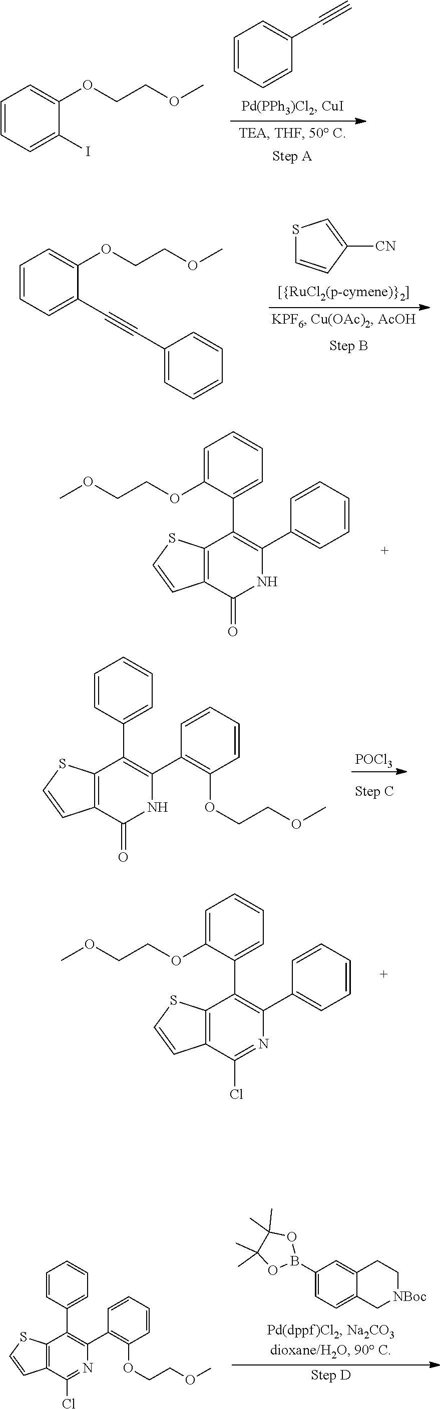

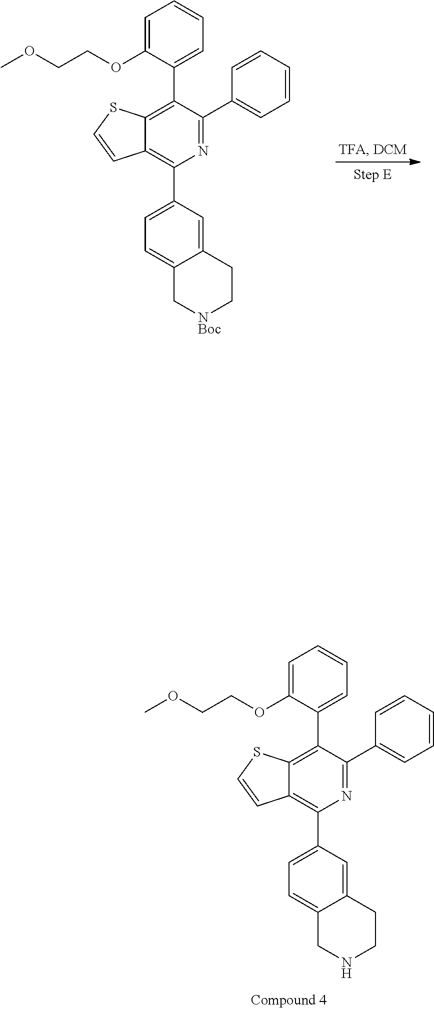 [SMILES: COCCOc1ccccc1-c1c(-c2ccccc2)nc(-c2ccc3c(c2)CCN(C(=O)OC(C)(C)C)C3)c2ccsc12 ; COCCOc1ccccc1-c1c(-c2ccccc2)nc(-c2ccc3c(c2)CCNC3)c2ccsc12]

Step A: Preparation of 1-(2-methoxyethoxy)-2-(phenylethynyl)benzene: To a solution of 1-iodo-2-(2-methoxyethoxy)benzene (300 mg, 1.08 mmol), phenylacetylene (0.120 mL, 1.13 mmol), triethylamine (0.75 mL, 5.4 mmol) and CuI (41 mg, 0.22 mmol) in THF (7.5 mL) was added Pd(PPh₃)₂Cl₂ (76 mg, 0.11 mmol). The reaction mixture was stirred at ambient temperature for 2 hours. The reaction was then heated to 50° C. and stirred for 4 hours under nitrogen. The reaction was quenched with water and extracted with EtOAc (x3). The combined organic layers were washed with brine, dried over Na₂SO₄, filtered and the filtrate was concentrated by rotary evaporation. The product was purified by column chromatography on silica gel (EtOAc/petroleum ether: 1/15) to afford 1-(2-methoxyethoxy)-2-(2-phenylethynyl)benzene (237 mg, 87% yield). ¹H NMR (400 MHz, CDCl₃): δ 7.47-7.50 (m, 3H), 7.25-7.36 (m, 4H), 6.90-6.97 (m, 2H), 4.22 (t, J=4.8 Hz, 2H), 3.84 (t, J=4.8 Hz, 2H), 3.51 (s, 3H).

Step B: Preparation of 6-[2-(2-methoxyethoxy)phenyl]-7-phenyl-5H-thieno[3,2-c]pyridin-4-one and 7-[2-(2-methoxyethoxy)phenyl]-6-phenyl-5H-thieno[3,2-c]pyridin-4-one: To a solution of 1-(2-methoxyethoxy)-2-(2-phenylethynyl)benzene (230 mg, 0.912 mmol), thiophene-3-carbonitrile (99 mg, 0.91 mmol), potassium hexafluorophosphate (34 mg, 0.18 mmol) and cupric acetate monohydrate (50 mg, 0.27 mmol) in acetic acid (10 mL) was added [{RuCl₂(p-cymene)}₂](28 mg, 0.046 mmol). The reaction was stirred at 120° C. for 10 hours. The reaction was concentrated to dryness and the residue was taken up in EtOAc. The organic layers were washed with water and brine, dried and concentrated. The crude was purified by column chromatography (5% MeOH in DCM) to afford a mixture of 6-[2-(2-methoxyethoxy)phenyl]-7-phenyl-5H-thieno[3,2-c]pyridin-4-one and 7-[2-(2-methoxyethoxy)phenyl]-6-phenyl-5H-thieno [3,2-c]pyridin-4-one (201 mg, 58% yield). LCMS ESI (+) m/z 378.1 (M+H).

Step C: Preparation of 4-chloro-6-[2-(2-methoxyethoxy)phenyl]-7-phenyl-thieno[3,2-c]pyridine and 4-chloro-7-[2-(2-methoxyethoxy)phenyl]-6-phenyl-thieno[3,2-c]pyridine: A solution of 6-[2-(2-methoxyethoxy)phenyl]-7-phenyl-5H-thieno[3,2-c]pyridin-4-one and 7-[2-(2-methoxyethoxy)phenyl]-6-phenyl-5H-thieno[3,2-c]pyridin-4-one (201 mg, 0.533 mmol) in POCl₃ (5.0 mL) was stirred at 100° C. for 3 hours. The reaction was concentrated to dryness and the residue was taken up in EtOAc. The organic layers were washed with saturated sodium bicarbonate solution and saturated brine solution, dried and concentrated. The crude was then purified by column chromatography (20% EtOAc in hexanes) to afford a mixture of two isomers, which was further purified by reverse phase HPLC to give 4-chloro-6-[2-(2-methoxyethoxy)phenyl]-7-phenyl-thieno[3,2-c]pyridine (41 mg, 19% yield) and 4-chloro-7-[2-(2-methoxyethoxy)phenyl]-6-phenyl-thieno[3,2-c]pyridine (64 mg, 30% yield). LCMS ESI (+) m/z 396.1 (M+H).

Step D: Preparation of tert-butyl 6-[7-[2-(2-methoxyethoxy)phenyl]-6-phenyl-thieno[3,2-c]pyridin-4-yl]-3,4-dihydro-1H-isoquinoline-2-carboxylate: To a suspension of a mixture of 4-chloro-6-[2-(2-methoxyethoxy)phenyl]-7-phenyl-thieno[3,2-c]pyridine and 4-chloro-7-[2-(2-methoxyethoxy)phenyl]-6-phenyl-thieno[3,2-c]pyridine (18 mg, 0.046 mmol), tert-butyl 6-(4,4,5,5-tetramethyl-1,3,2-dioxaborolan-2-yl)-3,4-dihydro-1H-isoquinoline-2-carboxylate (20 mg, 0.056 mmol) and sodium carbonate (14 mg, 0.13 mmol) in 1,4-dioxane (2 mL) and water (0.2 mL) was added 1,1′-bis(diphenylphosphino)ferrocenedichloropalladium(II) dichloromethane complex (4.0 mg, 0.0055 mmol). The mixture was stirred at 85° C. for 4 hours under Ar. The mixture was concentrated and purified by preparative TLC (EtOAc/petroleum ether: 1/4) to give tert-butyl 6-[7-[2-(2-methoxyethoxy)phenyl]-6-phenyl-thieno[3,2-c]pyridin-4-yl]-3,4-dihydro-1H-isoquinoline-2-carboxylate (22 mg, 82%) as a light-yellow solid.

Step E: Preparation of 7-[2-(2-methoxyethoxy) phenyl]-6-phenyl-4-(1,2,3,4-tetrahydroisoquinolin-6-yl)thieno[3,2-c]pyridine: To a solution of tert-butyl 6-[7-[2-(2-methoxyethoxy)phenyl]-6-phenyl-thieno[3,2-c]pyridin-4-yl]-3,4-dihydro-1H-isoquinoline-2-carboxylate (22 mg, 0.037 mmol) in DCM (3 mL) was added TFA (1 mL). The mixture was stirred at 10° C. for 2 hours. The mixture was concentrated and purified by reverse phase preparative-HPLC to give 7-[2-(2-methoxyethoxy)phenyl]-6-phenyl-4-(1,2,3,4-tetrahydroisoquinolin-6-yl)thieno[3,2-c]pyridine (15 mg, 83% yield) as a trifluoroacetic acid salt. LCMS ESI (+) m/z 493.2 (M+H). ¹H NMR (400 MHz, CD₃OD) δ: 7.95 (d, J=4.8 Hz, 1H), 7.85-7.88 (m, 2H), 7.72 (d, J=5.6 Hz, 1H), 7.47-7.55 (m, 3H), 7.42 (t, J=8.0 Hz, 1H), 7.24-7.38 (m, 4H), 7.08 (d, J=8.4 Hz, 1H), 7.02 (t, J=7.4 Hz, 1H), 4.52 (s, 2H), 4.00-4.08 (m, 1H), 3.84-3.88 (m, 1H), 3.60 (t, J=6.2 Hz, 2H), 3.42-3.53 (m, 2H), 3.15 (s, 3H).

### Synthetic Example 3: Synthesis of 3-[7-[4-fluoro-2-(2-methoxyethoxy)phenyl-4-(1,2,3,4-tetrahydroisoquinolin-6-yl)thieno[3,2-c]pyridin-6-yl]benzonitrile (Compound 8)

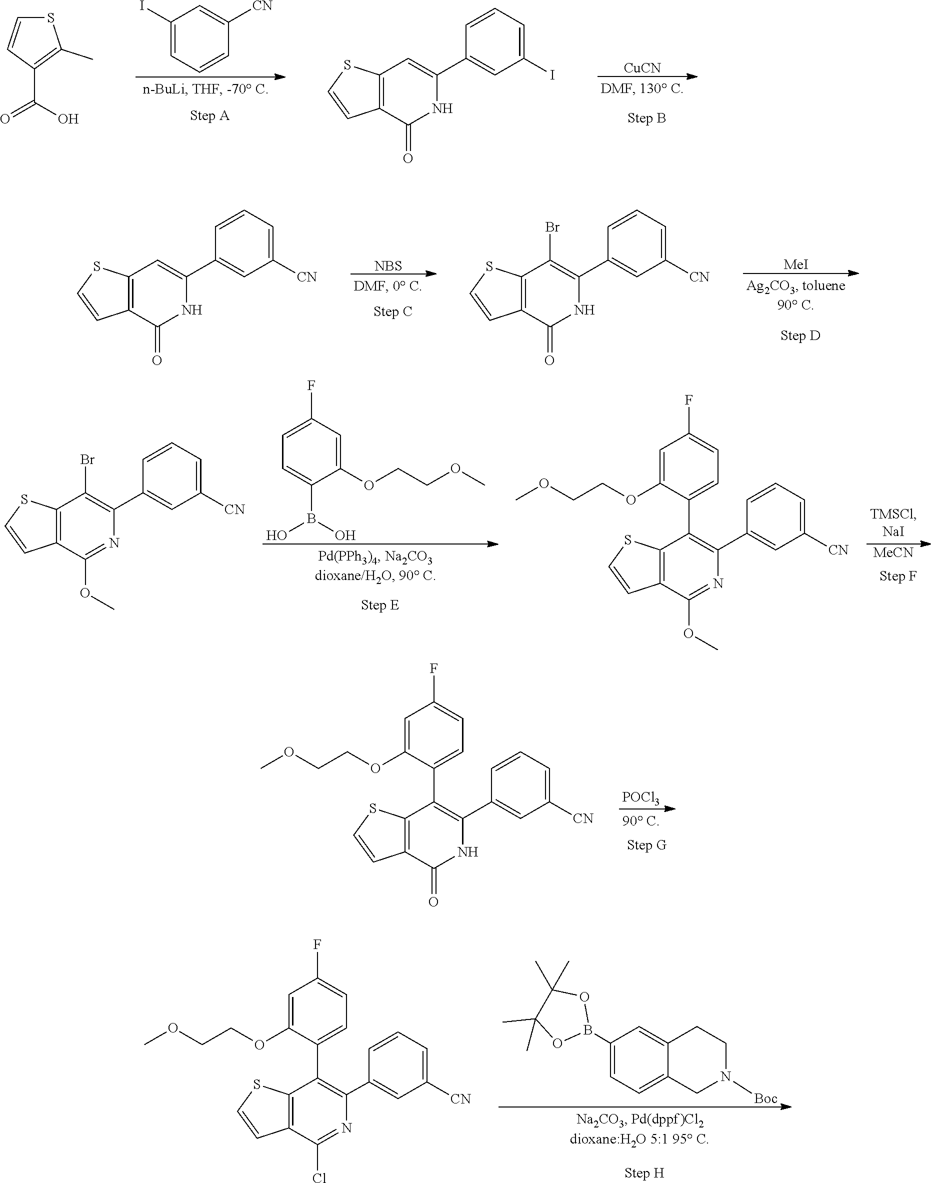

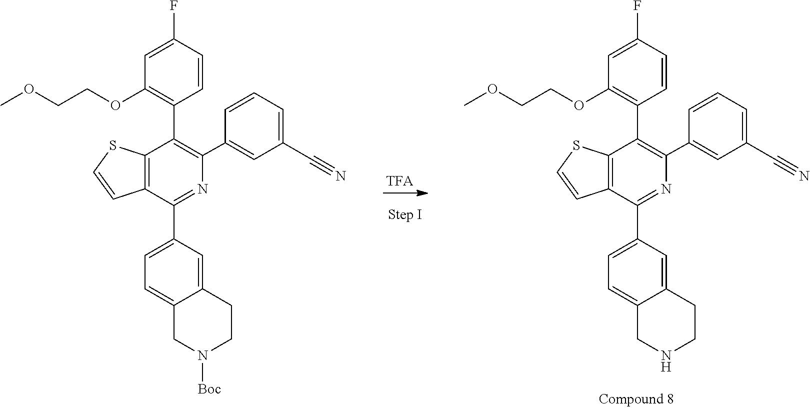 [SMILES: COCCOc1cc(F)ccc1-c1c(-c2cccc(C#N)c2)nc(-c2ccc3c(c2)CCN(C(=O)OC(C)(C)C)C3)c2ccsc12 ; COCCOc1cc(F)ccc1-c1c(-c2cccc(C#N)c2)nc(-c2ccc3c(c2)CCNC3)c2ccsc12]

Step A: Preparation of 6-(3-iodophenyl)-5H-thieno[3,2-c]pyridin-4-one: To a solution of 2-methylthiophene-3-carboxylic acid (2.00 g, 14.1 mmol) in THF (20 mL) was added n-butyllithium (12.0 mL, 2.5M in hexanes, 30.0 mmol) dropwise under Ar at −78° C. The mixture was stirred at −60° C. for 0.5 h under Ar. 3-iodobenzonitrile (3.50 g, 15.3 mmol) was then added at −60° C. The temperature was allowed to warm to 0° C., and the resulting mixture was stirred at 0° C. for additional 1 hour. The mixture was poured into water and the pH was adjusted to 5 with 1N HCl. The mixture was concentrated to remove THF and diluted with water. The solid was filtered, washed with water (20 mL) and EtOAc (20 mL), then dried in vacuo to give the crude product 6-(3-iodophenyl)-5H-thieno[3,2-c]pyridin-4-one (2.30 g, 46% yield) as a yellow solid. ¹H NMR (400 MHz, CDCl₃): δ 8.14 (s, 1H), 7.77-7.83 (m, 2H), 7.64 (d, J=5.2 Hz, 1H), 7.51 (d, J=5.2 Hz, 1H), 7.27-7.31 (m, 2H).

Step B: Preparation of 3-(4-oxo-5H-thieno[3,2-c]pyridin-6-yl)benzonitrile: A suspension of copper(I) cyanide (76 mg, 0.85 mmol) and 6-(3-iodophenyl)-5H-thieno[3,2-c]pyridin-4-one (200 mg, 0.566 mmol) in DMF (5 mL) was stirred at 130° C. for 16 hours. The mixture was allowed to cool to ambient temperature. Ammonia (1 mL) was added, and the mixture was stirred for additional 2 hours. The pH was adjusted to 6 with 3 N HCl. The solid was collected by filtration, and then washed with water and EtOAc/petroleum ether (1/1). The solid was dried in vacuo to give 3-(4-oxo-5H-thieno[3,2-c]pyridin-6-yl)benzonitrile (110 mg, 77% yield) as a yellow solid. LCMS ESI (+) m/z 253.0 (M+H).

Step C: Preparation of 3-(7-bromo-4-oxo-5H-thieno[3,2-c]pyridin-6-yl)benzonitrile: To a solution of crude 3-(4-oxo-5H-thieno[3,2-c]pyridin-6-yl)benzonitrile (1.27 g, 5.03 mmol) in DMF (15 mL) was added N-bromosuccinimide (941 mg, 5.29 mmol) at 0° C. The mixture was stirred at 0° C. for 1 h. The mixture was poured into water and the solid was filtered, washed with water and EtOAc/petroleum ether (1/1), then dried in vacuo to give 3-(7-bromo-4-oxo-5H-thieno[3,2-c]pyridin-6-yl)benzonitrile (870 mg, 52% yield) as a yellow solid. LCMS ESI (+) m/z 331, 333 (M+H).

Step D: Preparation of 3-(7-bromo-4-methoxy-thieno[3,2-c]pyridin-6-yl)benzonitrile: To a suspension of 3-(7-bromo-4-oxo-5H-thieno[3,2-c]pyridin-6-yl)benzonitrile (870 mg, 2.63 mmol) and silver carbonate (761 mg, 2.76 mmol) in toluene (20 mL) was added methyl iodide (0.41 mL, 6.6 mmol). The mixture was stirred at 90° C. for 16 hours in a sealed tube. The mixture was filtered through Celite® and washed with EtOAc. The filtrate was concentrated and the residue was purified by column chromatography (EtOAc/petroleum ether: 1/5) to give 3-(7-bromo-4-methoxy-thieno[3,2-c]pyridin-6-yl)benzonitrile (592 mg, 65% yield) as a light-yellow solid.

Step E: Preparation of 3-[7-[4-fluoro-2-(2-methoxyethoxy)phenyl]-4-methoxy-thieno[3,2-c]pyridin-6-yl]benzonitrile: To a solution of 3-(7-bromo-4-methoxy-thieno[3,2-c]pyridin-6-yl)benzonitrile (290 mg, 0.840 mmol) in 1,4-dioxane (5 mL) and water (1 mL) were added [4-fluoro-2-(2-methoxyethyl)phenyl]boronic acid (216 mg, 1.09 mmol), 1,1′-bis(diphenylphosphino)ferrocenedichloropalladium(II) dichloromethane complex (61 mg, 0.084 mmol) and sodium carbonate (178 mg, 1.68 mmol). The mixture was stirred at 100° C. for 4 hours. After cooling, the mixture was poured into water (20 mL) and the product was extracted with ethyl acetate (40 mL). The combined organic layers were washed with brine, dried over Na₂SO₄, filtered and concentrated. The residue was purified by preparative TLC (petroleum ether:EtOAc=3:1) to give 3-[7-[4-fluoro-2-(2-methoxyethoxy)phenyl]-4-methoxy-thieno[3,2-c]pyridin-6-yl]benzonitrile (280 mg, 77% yield). LCMS ESI (+) m/z 435 (M+H).

Step F: Preparation of 3-[7-[4-fluoro-2-(2-methoxyethoxy)phenyl]-4-oxo-5H-thieno[3,2-c]pyridin-6-yl]benzonitrile: To a solution of 3-[7-[4-fluoro-2-(2-methoxyethoxy)phenyl]-4-methoxy-thieno[3,2-c]pyridin-6-yl]benzonitrile (205 mg, 0.472 mmol) in acetonitrile (20 mL) were added sodium iodide (212 mg, 1.42 mmol) and trimethylchlorosilane (0.180 mL, 1.42 mmol). The mixture was stirred at 0° C. for 2 hours. The mixture was poured into water (20 mL), and the solid was filtered to give 3-[7-[4-fluoro-2-(2-methoxyethoxy)phenyl]-4-oxo-5H-thieno[3,2-c]pyridin-6-yl]benzonitrile (150 mg, 76% yield). LCMS ESI (+) m/z 421 (M+H).

Step G: Preparation of 3-[4-chloro-7-[4-fluoro-2-(2-methoxyethoxy) phenyl]thieno[3,2-c]pyridin-6-yl]benzonitrile: A solution of 3-[7-[4-fluoro-2-(2-methoxyethoxy)phenyl]-4-oxo-5H-thieno[3,2-c]pyridin-6-yl]benzonitrile (150 mg, 0.357 mmol) in phosphorus oxychloride (3.00 mL, 32.2 mmol) was stirred at 90° C. for 1 hour. After cooling, the mixture was poured into ice-water (20 mL). NaHCO₃ was carefully added to adjust pH to 8. The product was extracted with EtOAc (50 mL). The combined organic layers were washed with brine, dried over Na₂SO₄, filtered and concentrated. The residue was purified by preparative TLC (petroleum ether:EA=5:1) to give 3-[4-chloro-7-[4-fluoro-2-(2-methoxyethoxy) phenyl]thieno[3,2-c]pyridin-6-yl]benzonitrile (130 mg, 83% yield). LCMS ESI (+) m/z 439.0 (M+H).

Step H: Preparation of tert-butyl 6-[6-(3-cyanophenyl)-7-[4-fluoro-2-(2-methoxyethoxy)phenyl]thieno[3,2-c]pyridin-4-yl]-3,4-dihydro-1H-isoquinoline-2-carboxylate: To a solution of 3-[4-chloro-7-[4-fluoro-2-(2-methoxyethoxy)phenyl]thieno[3,2-c]pyridin-6-yl]benzonitrile (130 mg, 0.296 mmol) in 1,4-dioxane (2 mL) and water (0.2 mL) were added tert-butyl 6-(4,4,5,5-tetramethyl-1,3,2-dioxaborolan-2-yl)-3,4-dihydro-1H-isoquinoline-2-carboxylate (138 mg, 0.385 mmol), sodium carbonate (63 mg, 0.59 mmol) and 1,1′-bis(diphenylphosphino)ferrocenedichloro palladium(II) dichloromethane complex (22 mg, 0.030 mmol). The mixture was stirred at 95° C. for 2 hours under Ar. After cooling, the mixture was poured into water (20 mL) and extracted with ethyl acetate (40 mL). The combined organic layers were washed with brine, dried over Na₂SO₄, filtered and concentrated. The residue was purified by preparative TLC (petroleum ether:EA=3:1) to give tert-butyl 6-[6-(3-cyanophenyl)-7-[4-fluoro-2-(2-methoxyethoxy)phenyl]thieno[3,2-c]pyridin-4-yl]-3,4-dihydro-1H-isoquinoline-2-carboxylate (160 mg, 85% yield). LCMS ESI (+) m/z 636 (M+H).

Step I: Preparation of 3-[7-[4-fluoro-2-(2-methoxyethoxy)phenyl-4-(1,2,3,4-tetrahydroisoquinolin-6-yl)thieno[3,2-c]pyridin-6-yl]benzonitrile: To a solution of tert-butyl 6-[6-(3-cyanophenyl)-7-[4-fluoro-2-(2-methoxyethoxy)phenyl]thieno[3,2-c]pyridin-4-yl]-3,4-dihydro-1H-isoquinoline-2-carboxylate (17 mg, 0.027 mmol) in DCM (4 mL) was added trifluoroacetic acid (1.0 mL, 13 mmol). The mixture was stirred at 25° C. for 1 hour. The mixture was concentrated and the residue was purified by reverse phase preparative-HPLC to give 3-[7-[4-fluoro-2-(2-methoxyethoxy)phenyl-4-(1,2,3,4-tetrahydroisoquinolin-6-yl)thieno[3,2-c]pyridin-6-yl]benzonitrile (4.5 mg, 31% yield) as a trifluoroacetic acid salt. LCMS ESI (+) m/z 536 (M+H). 1H NMR (400 MHz, CD₃OD) δ 7.89 (s, 1H), 7.81-7.86 (m, 3H), 7.68-7.72 (m, 2H), 7.63 (d, J=8 Hz, 1H), 7.39-7.47 (m, 2H), 7.32-7.35 (m, 1H), 6.90 (dd, J=11.1, 2.2 Hz, 1H), 6.79-6.83 (m, 1H), 4.49 (s, 2H), 3.98-4.01 (m, 1H), 3.83-3.86 (m, 1H), 3.60 (t, J=6.4 Hz, 2H), 3.44 (t, J=4.8, 2H), 3.26-3.30 (m, 2H), 3.15 (s, 3H).

### Synthetic Example 4: Synthesis of N-[[3-[7-[4-fluoro-2-(2-methoxyethoxy)phenyl]-4-(1,2,3,4-tetrahydroisoquinolin-6-yl)thieno[3,2-c]pyridin-6-yl]phenyl]methyl]prop-2-enamide (Compound 11)

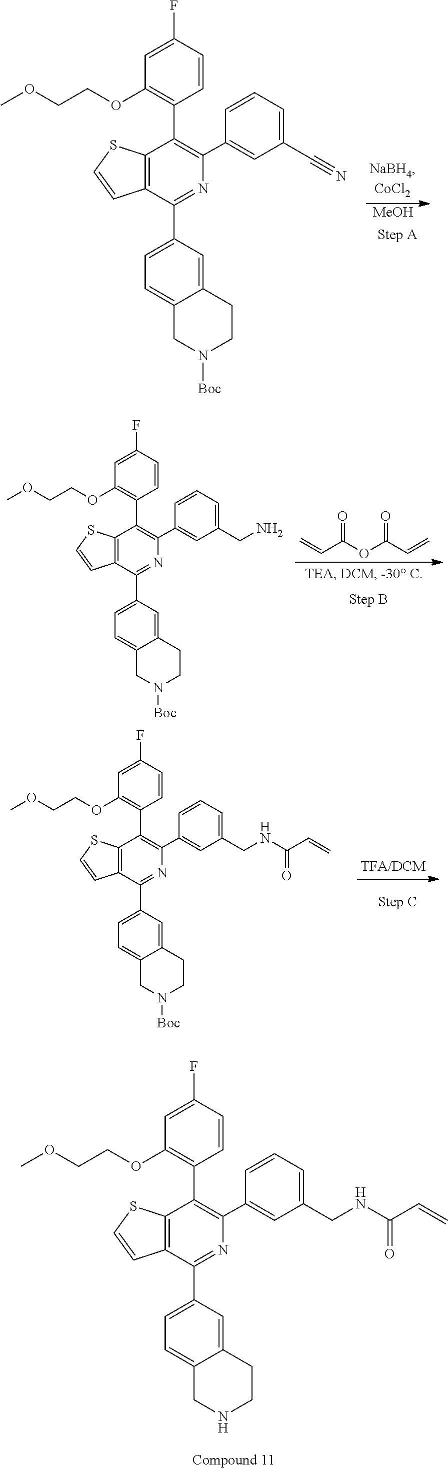

Step A: Preparation of tert-butyl 6-[6-[3-(aminomethyl)phenyl]-7-[4-fluoro-2-(2-methoxyethoxy)phenyl]thieno[3,2-c]pyridin-4-yl]-3,4-dihydro-1H-isoquinoline-2-carboxylate: To a solution of tert-butyl 6-[6-(3-cyanophenyl)-7-[4-fluoro-2-(2-methoxyethoxy)phenyl]thieno[3,2-c]pyridin-4-yl]-3,4-dihydro-1H-isoquinoline-2-carboxylate (178 mg, 0.280 mmol) in methanol (5 mL) were added cobaltous chloride (73 mg, 0.56 mmol) and sodium borohydride (106 mg, 2.80 mmol). The mixture was stirred at 25° C. for 12 hours. The mixture was poured into water (20 mL) and extracted with ethyl acetate (40 mL). The combined organic layers were washed with brine, dried over Na₂SO₄ and concentrated. The residue was purified by preparative TLC (DCM:MeOH=20:1) to give tert-butyl 6-[6-[3-(aminomethyl)phenyl]-7-[4-fluoro-2-(2-methoxyethoxy)phenyl]thieno[3,2-c]pyridin-4-yl]-3,4-dihydro-1H-isoquinoline-2-carboxylate (135 mg, 75% yield). LCMS ESI (+) m/z 640.3 (M+H).

Step B: Preparation of tert-butyl 6-[7-[4-fluoro-2-(2-methoxyethoxy)phenyl]-6-[3-[(prop-2-enoylamino)methyl]phenyl]thieno[3,2-c]pyridin-4-yl]-3,4-dihydro-1H-isoquinoline-2-carboxylate: To the solution of tert-butyl 6-[6-[3-(aminomethyl)phenyl]-7-[4-fluoro-2-(2-methoxyethoxy)phenyl]thieno[3,2-c]pyridin-4-yl]-3,4-dihydro-1H-isoquinoline-2-carboxylate (12 mg, 0.019 mmol) in DCM (2 mL) was added triethylamine (0.0078 mL, 0.056 mmol). The mixture was cooled to −20° C. To this was added a solution of prop-2-enoyl prop-2-enoate (2.8 mg, 0.023 mmol) in DCM. The mixture was stirred for 15 min at −20° C. The reaction was quenched with H₂O (10 mL) and extracted with EtOAc (15 mL×3). The organic layers were concentrated and the residue was purified by preparative TLC (EtOAc/petroleum ether: 50 to 100% EtOAc) to give tert-butyl 6-[7-[4-fluoro-2-(2-methoxyethoxy)phenyl]-6-[3-[(prop-2-enoylamino)methyl]phenyl]thieno[3,2-c]pyridin-4-yl]-3,4-dihydro-1H-isoquinoline-2-carboxylate (12 mg, 92% yield). LCMS ESI (+) m/z 694.3 (M+H).

Step C: Preparation of N-[[3-[7-[4-fluoro-2-(2-methoxyethoxy)phenyl]-4-(1,2,3,4-tetrahydroisoquinolin-6-yl)thieno[3,2-c]pyridin-6-yl]phenyl]methyl]prop-2-enamide: To a solution of tert-butyl 6-[7-[4-fluoro-2-(2-methoxyethoxy)phenyl]-6-[3-[(prop-2-enoylamino)methyl]phenyl]thieno[3,2-c]pyridin-4-yl]-3,4-dihydro-1H-isoquinoline-2-carboxylate (14 mg, 0.020 mmol) in dichloromethane (3 mL) was added trifluoroacetic acid (1 mL). The resulting mixture was stirred for 1 hour. The solvent was concentrated under reduced pressure and the residue was purified by reverse phase preparative-HPLC to afford N-[[3-[7-[4-fluoro-2-(2-methoxyethoxy)phenyl]-4-(1,2,3,4-tetrahydroisoquinolin-6-yl)thieno[3,2-c]pyridin-6-yl]phenyl]methyl]prop-2-enamide (2.3 mg, 19% yield) as a trifluoroacetic acid salt. LCMS ESI (+) m/z 593.2 (M+H). ¹H NMR (400 MHz, CD₃OD) δ 7.91 (d, J=5.6 Hz, 1H), 7.84-7.87 (m, 2H), 7.70 (d, J=5.6 Hz, 1H), 7.45-7.51 (m, 2H), 7.36-7.40 (m, 1H), 7.22-7.32 (m, 3H), 6.88 (dd, J=11.1, 2.2 Hz, 1H), 6.75 (td, J=8.3, 2.3 Hz, 1H), 6.25 (d, J=6.0 Hz, 2H), 5.70 (t, J=6.0 Hz, 1H), 4.51 (s, 2H), 4.40 (s, 2H), 3.96-4.08 (m, 1H), 3.78-3.91 (m, 1H), 3.60 (t, J=6.3 Hz, 1H), 3.44-3.47 (m, 2H), 3.13 (s, 3H).

### Synthetic Example 5: Synthesis of N-[3-[7-[4-fluoro-2-(2-methoxyethoxy)phenyl]-4-(1,2,3,4-tetrahydroisoquinolin-6-yl)thieno[3,2-c]pyridin-6-yl]phenyl]prop-2-enamide (Compound 12)

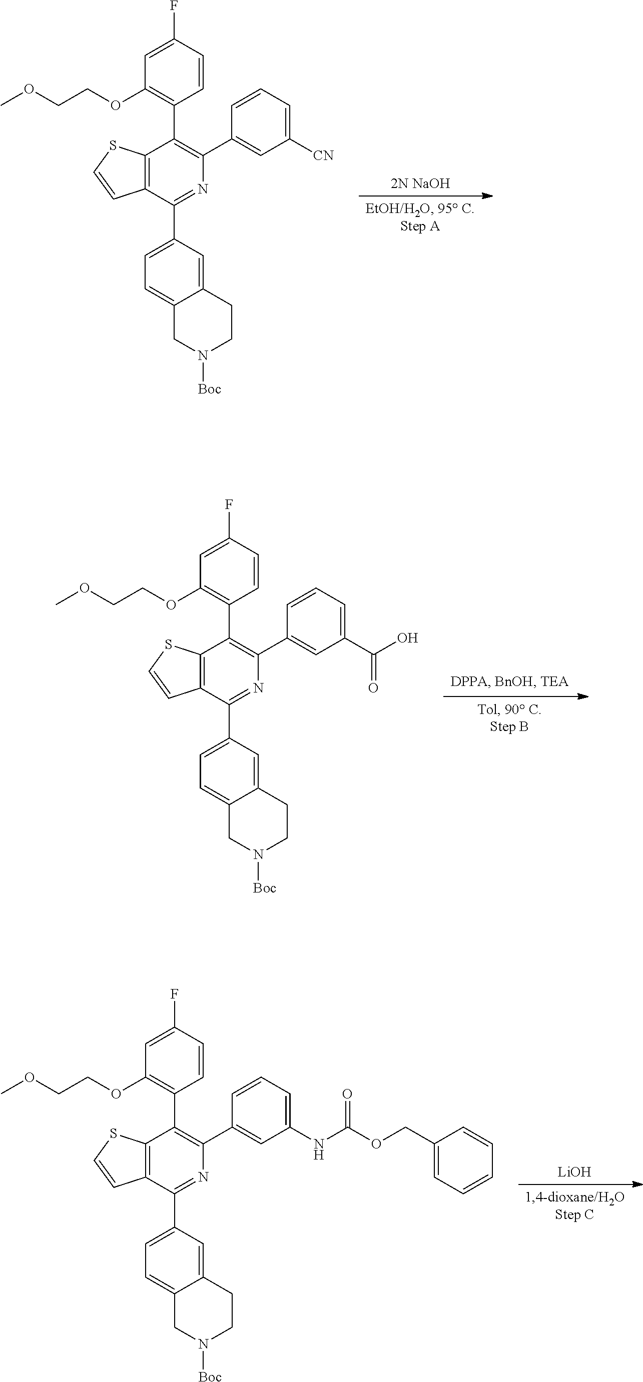

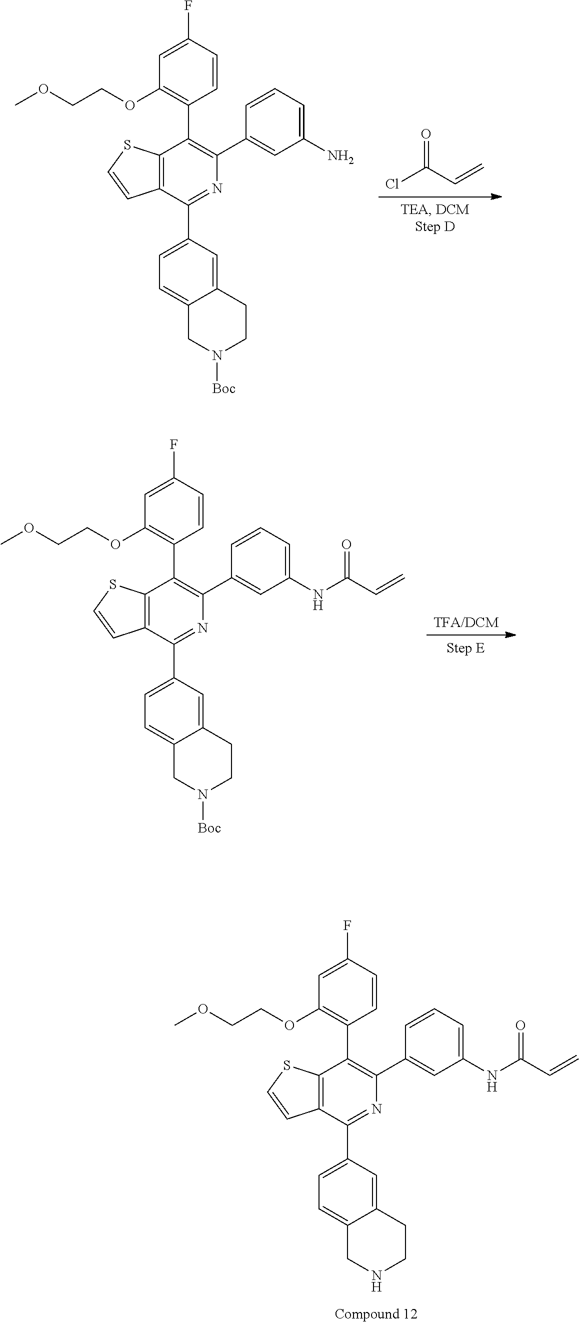

Step A: Preparation of 3-[4-(2-tert-butoxycarbonyl-3,4-dihydro-1H-isoquinolin-6-yl)-7-[4-fluoro-2-(2-methoxyethoxy)phenyl]thieno[3,2-c]pyridin-6-yl]benzoic acid: A solution of tert-butyl 6-[6-(3-cyanophenyl)-7-[4-fluoro-2-(2-methoxyethoxy)phenyl]thieno[3,2-c]pyridin-4-yl]-3,4-dihydro-1H-isoquinoline-2-carboxylate (350 mg, 0.551 mmol) in ethanol (3 mL) and NaOH (2 N, 3 mL) was stirred at 95° C. for 12 h. The mixture was diluted with water (20 mL), and 1N HCl was added to adjust pH to 2. The product was extracted with EtOAc, washed with brine, dried over Na₂SO₄, filtered and concentrated to dryness to give 3-[4-(2-tert-butoxycarbonyl-3,4-dihydro-1H-isoquinolin-6-yl)-7-[4-fluoro-2-(2-methoxyethoxy)phenyl]thieno[3,2-c]pyridin-6-yl]benzoic acid (310 mg, 86% yield) as a white solid. LCMS ESI (+) m/z 655.3 (M+H).

Step B: Preparation of tert-butyl 6-[6-[3-(benzyloxycarbonylamino)phenyl]-7-[4-fluoro-2-(2-methoxyethoxy)phenyl]thieno[3,2-c]pyridin-4-yl]-3,4-dihydro-1H-isoquinoline-2-carboxylate: To a solution of 3-[4-(2-tert-butoxycarbonyl-3,4-dihydro-1H-isoquinolin-6-yl)-7-[4-fluoro-2-(2-methoxyethoxy)phenyl]thieno[3,2-c]pyridin-6-yl]benzoic acid (42 mg, 0.064 mmol), triethylamine (0.090 mL, 0.64 mmol) and benzyl alcohol (0.033 mL, 0.32 mmol) in dry toluene (3 mL) was added [azido(phenoxy)phosphoryl]oxybenzene (89 mg, 0.32 mmol). The mixture was stirred at 90° C. for 2 hours. The mixture was concentrated and purified by preparative TLC (EtOAc/petroleum ether: 1/2) to give tert-butyl 6-[6-[3-(benzyloxycarbonylamino)phenyl]-7-[4-fluoro-2-(2-methoxyethoxy)phenyl]thieno[3,2-c]pyridin-4-yl]-3,4-dihydro-1H-isoquinoline-2-carboxylate (36 mg, 74% yield). LCMS ESI (+) m/z 760.2 (M+H).

Step C: Preparation of tert-butyl 6-[6-(3-aminophenyl)-7-[4-fluoro-2-(2-methoxyethoxy)phenyl]thieno[3,2-c]pyridin-4-yl]-3,4-dihydro-1H-isoquinoline-2-carboxylate: A solution of tert-butyl 6-[6-[3-(benzyloxycarbonylamino)phenyl]-7-[4-fluoro-2-(2-methoxyethoxy)phenyl]thieno[3,2-c]pyridin-4-yl]-3,4-dihydro-1H-isoquinoline-2-carboxylate (36 mg, 0.047 mmol) and lithium hydroxide monohydrate (40 mg, 0.95 mmol) in 1,4-dioxane (1 mL) and water (1 mL) was stirred at 95° C. for 16 hours under Ar. The mixture was poured into water. The mixture was extracted with EtOAc, dried over anhydrous Na₂SO₄, filtered and concentrated. The residue was purified by preparative TLC (EtOAc/petroleum ether: 1/2) to give tert-butyl 6-[6-(3-aminophenyl)-7-[4-fluoro-2-(2-methoxyethoxy)phenyl]thieno[3,2-c]pyridin-4-yl]-3,4-dihydro-1H-isoquinoline-2-carboxylate (24 mg, 81% yield) as a white solid. LCMS ESI (+) m/z 626.3 (M+H).

Step D: Preparation of tert-butyl 6-[7-[4-fluoro-2-(2-methoxyethoxy)phenyl]-6-[3-(prop-2-enoylamino)phenyl]thieno[3,2-c]pyridin-4-yl]-3,4-dihydro-1H-isoquinoline-2-carboxylate: To a solution of tert-butyl 6-[6-(3-aminophenyl)-7-[4-fluoro-2-(2-methoxyethoxy)phenyl]thieno[3,2-c]pyridin-4-yl]-3,4-dihydro-1H-isoquinoline-2-carboxylate (12 mg, 0.019 mmol) and N,N-diisopropylethylamine (0.015 mL, 0.085 mmol) in DCM (2 mL) was added acryloyl chloride (0.0045 mL, 0.055 mmol) at −30° C. The mixture was stirred at 0° C. for 1 hour. The mixture was poured into NaHCO₃ aqueous solution and extracted with EtOAc. The combined organic layers were dried over anhydrous Na₂SO₄, concentrated and purified by preparative TLC (EtOAc/petroleum ether: 1/2) to give tert-butyl 6-[7-[4-fluoro-2-(2-methoxyethoxy)phenyl]-6-[3-(prop-2-enoylamino)phenyl]thieno[3,2-c]pyridin-4-yl]-3,4-dihydro-1H-isoquinoline-2-carboxylate (12 mg, 92% yield).

Step E: Preparation of N-[3-[7-[4-fluoro-2-(2-methoxyethoxy)phenyl]-4-(1,2,3,4-tetrahydroisoquinolin-6-yl)thieno[3,2-c]pyridin-6-yl]phenyl]prop-2-enamide: To a solution of tert-butyl 6-[7-[4-fluoro-2-(2-methoxyethoxy)phenyl]-6-[3-(prop-2-enoylamino)phenyl]thieno[3,2-c]pyridin-4-yl]-3,4-dihydro-1H-isoquinoline-2-carboxylate (12 mg, 0.018 mmol) in DCM (2 mL) was added TFA (0.5 mL). The mixture was stirred at 25° C. for 0.5 h. The mixture was concentrated and purified by reverse phase preparative-HPLC to give N-[3-[7-[4-fluoro-2-(2-methoxyethoxy)phenyl]-4-(1,2,3,4-tetrahydroisoquinolin-6-yl)thieno[3,2-c]pyridin-6-yl]phenyl]prop-2-enamide (7.4 mg, 72% yield) as a trifluoroacetic acid salt. LCMS ESI (+) m/z 580.2 (M+H). ¹H NMR (400 MHz, CD₃OD) δ 8.06 (s, 1H), 7.83-7.87 (m, 3H), 7.68 (d, J=5.6 Hz, 1H), 7.45-7.49 (m, 2H), 7.29 (dd, J=8.4, 6.8 Hz, 1H), 7.20 (t, J=7.8 Hz, 1H), 7.10-7.14 (m, 1H), 6.88 (dd, J=11.0, 1.0 Hz, 1H), 6.74-6.80 (m, 1H), 6.32-6.46 (m, 2H), 5.76 (dd, J=9.6, 2.1 Hz, 1H), 4.50 (s, 2H), 3.99-4.50 (m, 1H), 3.82-3.87 (m, 1H), 3.60 (t, J=6.4 Hz, 2H), 3.45-3.49 (m, 2H), 3.12 (s, 2H).

### Synthetic Example 6: Synthesis of N-[2-[7-[4-fluoro-2-(2-methoxyethoxy)phenyl]-4-(1,2,3,4-tetrahydroisoquinolin-6-yl)thieno[3,2-c]pyridin-6-yl]-3H-benzimidazol-5-yl]prop-2-enamide (Compound 23)

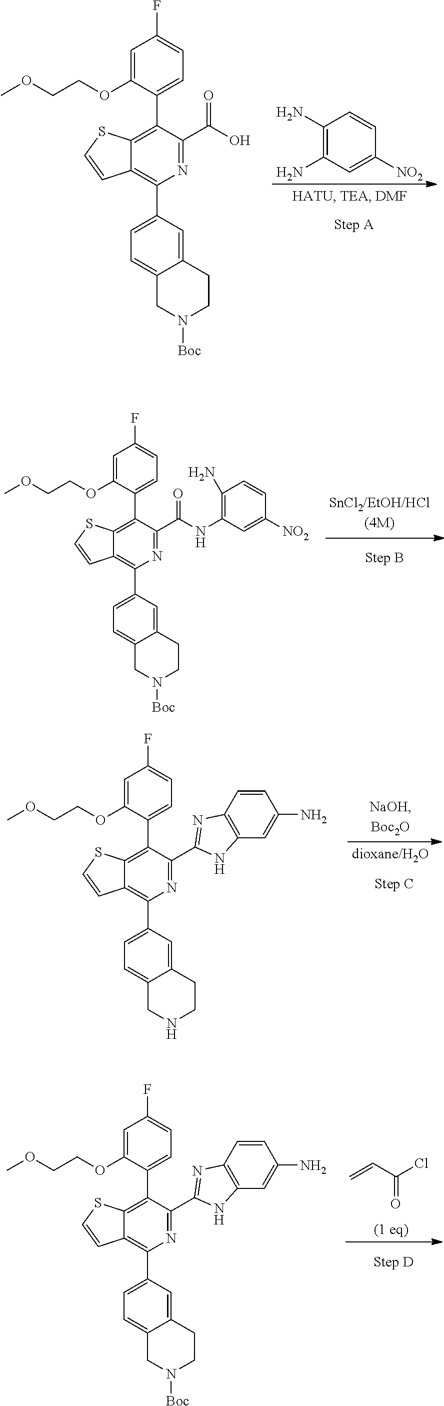

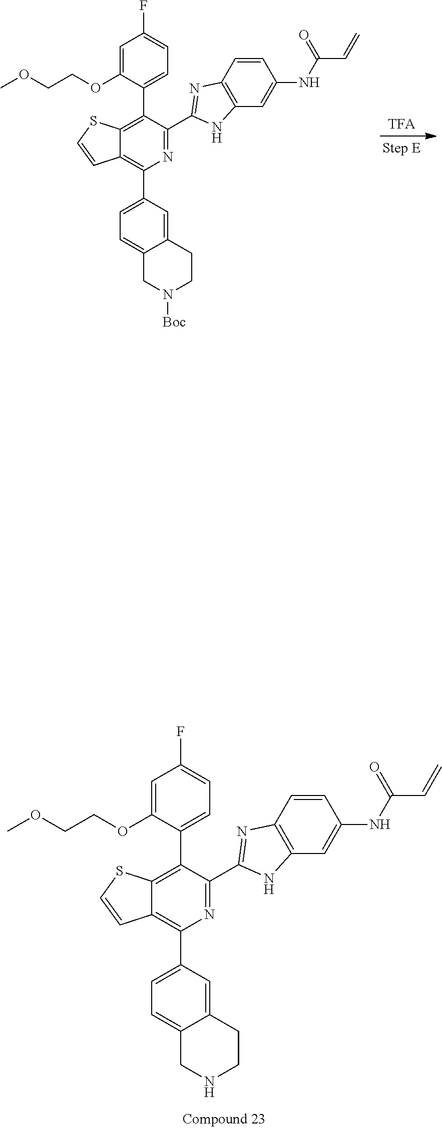

Step A: Preparation of tert-butyl 6-(6-((2-amino-5-nitrophenyl)carbamoyl)-7-(4-fluoro-2-(2-methoxyethoxy)phenyl)thieno[3,2-c]pyridin-4-yl)-3,4-dihydroisoquinoline-2(1H)-carboxylate: To a solution of 4-(2-tert-butoxycarbonyl-3,4-dihydro-1H-isoquinolin-6-yl)-7-[4-fluoro-2-(2-methoxyethoxy)phenyl]thieno[3,2-c]pyridine-6-carboxylic acid (100 mg, 0.173 mmol) in DMF (2 mL) were added HATU (99 mg, 0.26 mmol) and TEA (0.072 mL, 0.52 mmol). After the mixture was stirred at ambient temperature for 15 min, 4-nitrobenzene-1,2-diamine (32 mg, 0.21 mmol) was added. The resulting mixture was stirred at 30° C. for 2 hours. The reaction was quenched with water and extracted with EtOAc twice. The combined organic phases were washed with saturated NH₄Cl and brine, dried and concentrated. The residue was purified by preparative TLC (EtOAc/petroleum ether: 1/2) to afford tert-butyl 6-(6-((2-amino-5-nitrophenyl)carbamoyl)-7-(4-fluoro-2-(2-methoxyethoxy)phenyl)thieno[3,2-c]pyridin-4-yl)-3,4-dihydroisoquinoline-2(1H)-carboxylate (80 mg, 65% yield). LCMS ESI (+) m/z 714.2 (M+H).

Step B: Preparation of 2-(7-(4-fluoro-2-(2-methoxyethoxy)phenyl)-4-(1,2,3,4-tetrahydroisoquinolin-6-yl)thieno[3,2-c]pyridin-6-yl)-1H-benzo[d]imidazol-6-amine: To a solution of tert-butyl 6-[6-[(2-amino-5-nitro-phenyl)carbamoyl]-7-[4-fluoro-2-(2-methoxyethoxy)phenyl]thieno[3,2-c]pyridin-4-yl]-3,4-dihydro-1H-isoquinoline-2-carboxylate (73 mg, 0.10 mmol) in ethanol (1 mL) was added stannous chloride dihydrate (162 mg, 0.716 mmol). Concentrated hydrochloric acid (0.5 mL) was added to the reaction mixture. The reaction mixture was refluxed for 4 hours. Upon completion, the reaction mixture was basified with 30% sodium hydroxide solution. The mixture was extracted with dichloromethane (20 mL×3), dried over anhydrous Na₂SO₄, filtered and concentrated under reduced pressure. The crude was purified by preparative TLC (DCM/MeOH (containing 0.2% Et₃N): 20/1) to afford 2-(7-(4-fluoro-2-(2-methoxyethoxy)phenyl)-4-(1,2,3,4-tetrahydroisoquinolin-6-yl)thieno[3,2-c]pyridin-6-yl)-1H-benzo[d]imidazol-6-amine (60 mg, 93% yield). LCMS ESI (+) m/z 566.2 (M+H).

Step C: Preparation of tert-butyl 6-(6-(6-amino-1H-benzo[d]imidazol-2-yl)-7-(4-fluoro-2-(2-methoxyethoxy)phenyl)thieno[3,2-c]pyridin-4-yl)-3,4-dihydroisoquinoline-2(1H)-carboxylate: To a solution of 2-[7-[4-fluoro-2-(2-methoxyethoxy)phenyl]-4-(1,2,3,4-tetrahydroisoquinolin-6-yl)thieno[3,2-c]pyridin-6-yl]-3H-benzimidazol-5-amine (60 mg, 0.096 mmol) in 1,4-dioxane (4 mL) and water (2 mL) was added NaOH (19 mg, 0.48 mmol) and di-tert-butyl dicarbonate (21 mg, 0.096 mmol). The reaction was stirred at 30° C. for 1 hour. The reaction was quenched with water and extracted with DCM twice. The organic layers were washed with brine, dried and concentrated. The residue was purified by preparative TLC (EtOAc) to afford tert-butyl 6-(6-(6-amino-1H-benzo[d]imidazol-2-yl)-7-(4-fluoro-2-(2-methoxyethoxy)phenyl)thieno[3,2-c]pyridin-4-yl)-3,4-dihydroisoquinoline-2(1H)-carboxylate (65 mg, 82% yield). LCMS ESI (+) m/z 666.2 (M+H).

Step D: Preparation of tert-butyl 6-[7-[4-fluoro-2-(2-methoxyethoxy)phenyl]-6-[6-(prop-2-enoylamino)-1H-benzimidazol-2-yl]thieno[3,2-c]pyridin-4-yl]-3,4-dihydro-1H-isoquinoline-2-carboxylate: To a solution of tert-butyl 6-[6-(6-amino-1H-benzimidazol-2-yl)-7-[4-fluoro-2-(2-methoxyethoxy)phenyl]thieno[3,2-c]pyridin-4-yl]-3,4-dihydro-1H-isoquinoline-2-carboxylate (65 mg, 0.078 mmol) in DCM (3 mL) was added triethylamine (0.033 mL, 0.23 mmol). Solution of acryloyl chloride (0.0063 mL, 0.078 mmol) in DCM was added at −30° C. The mixture was stirred at 30° C. for 1 hour and then quenched with water and extracted with DCM twice. The organic layers were washed with brine, dried and concentrated. The residue was purified by preparative TLC (EtOAc) to afford tert-butyl 6-[7-[4-fluoro-2-(2-methoxyethoxy)phenyl]-6-[6-(prop-2-enoylamino)-1H-benzimidazol-2-yl]thieno[3,2-c]pyridin-4-yl]-3,4-dihydro-1H-isoquinoline-2-carboxylate (40 mg, 71% yield). LCMS ESI (+) m/z 720.3 (M+H).

Step E: Preparation of N-[2-[7-[4-fluoro-2-(2-methoxyethoxy)phenyl]-4-(1,2,3,4-tetrahydroisoquinolin-6-yl)thieno[3,2-c]pyridin-6-yl]-3H-benzimidazol-5-yl]prop-2-enamide: To a solution of tert-butyl 6-[7-[4-fluoro-2-(2-methoxyethoxy)phenyl]-6-[6-(prop-2-enoylamino)-1H-benzimidazol-2-yl]thieno[3,2-c]pyridin-4-yl]-3,4-dihydro-1H-isoquinoline-2-carboxylate (40 mg, 0.056 mmol) in DCM (3 mL) was added trifluoroacetic acid (1.0 mL, 13 mmol). The mixture was stirred at 30° C. for 1 h. The mixture was concentrated and purified by reverse phase preparative-HPLC to afford N-[2-[7-[4-fluoro-2-(2-methoxyethoxy)phenyl]-4-(1,2,3,4-tetrahydroisoquinolin-6-yl)thieno[3,2-c]pyridin-6-yl]-3H-benzimidazol-5-yl]prop-2-enamide (18 mg, 52% yield). LCMS ESI (+) m/z 620.2 (M+H). ¹H NMR (400 MHz, CD₃OD) δ 8.48 (s, 1H), 8.06 (d, J=5.6 Hz, 1H), 7.99-8.01 (m, 2H), 7.85 (d, J=5.4 Hz, 1H), 7.77 (t, J=7.6 Hz, 1H), 7.66 (d, J=8.9 Hz, 1H), 7.49-7.53 (m, 2H), 6.97-7.04 (m, 2H), 6.38-6.52 (m, 2H), 5.83 (dd, J=9.3, 2.3 Hz, 1H), 4.53 (s, 2H), 3.88-3.95 (m, 1H), 3.72-3.80 (m, 1H), 3.61 (t, J=6.3 Hz 2H), 3.20-3.30 (m, 4H), 2.96 (s, 3H).

### Synthetic Example 7: Synthesis of 1-[3-[4-[7-[4-fluoro-2-(2-methoxyethoxy)phenyl]-4-(1,2,3,4-tetrahydroisoquinolin-6-yl)thieno[3,2-c]pyridin-6-yl]pyrazol-1-yl]azetidin-1-yl]prop-2-en-1-one (Compound 44)

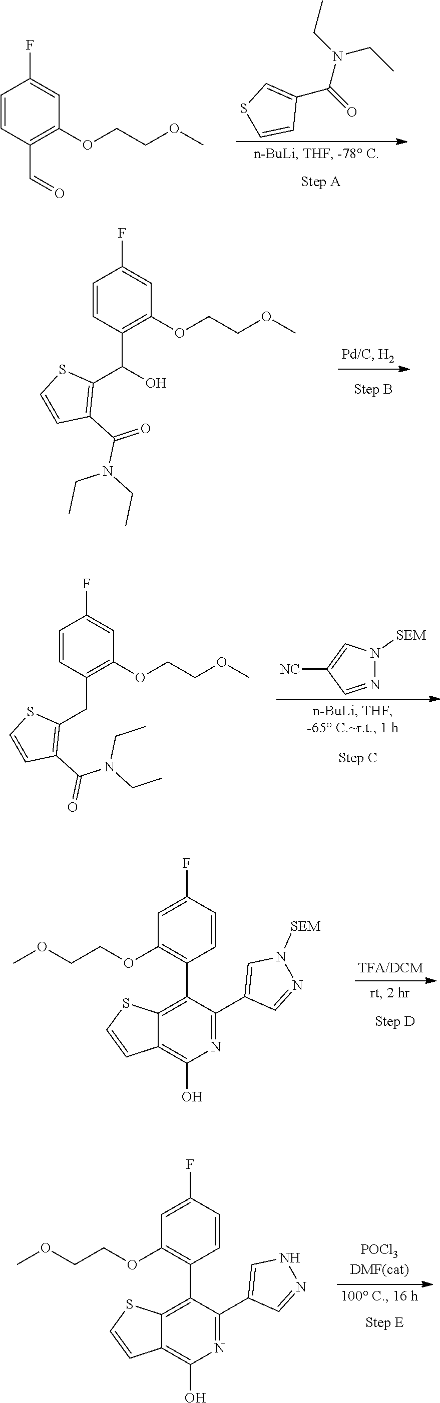

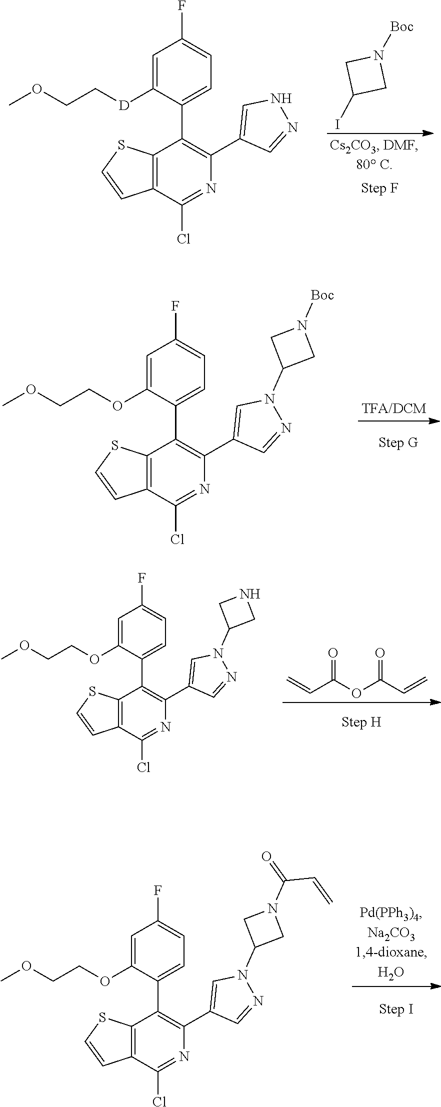

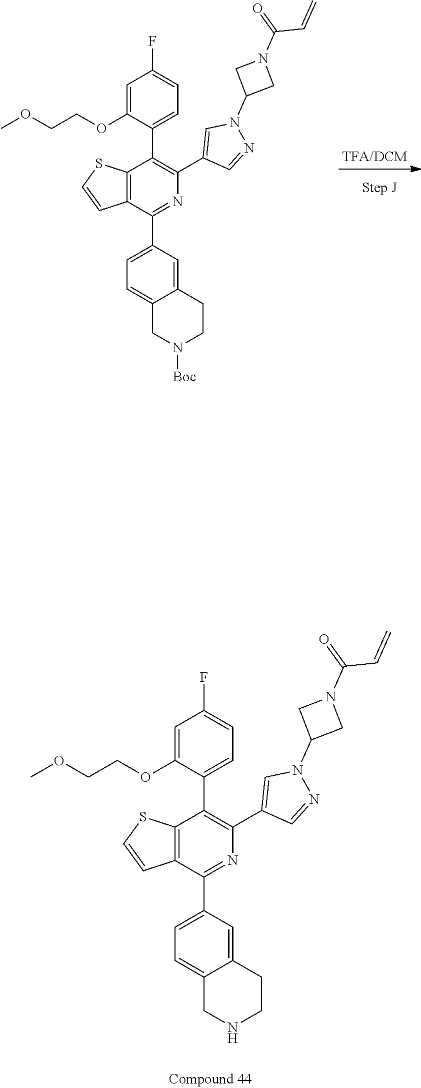

Step A: Preparation of N,N-diethyl-2-[[4-fluoro-2-(2-methoxyethoxy)phenyl]-hydroxy-methyl]thiophene-3-carboxamide: A solution of N,N-diethylthiophene-3-carboxamide (7.00 g, 38.2 mmol) in THF (50 mL) was cooled to −78° C. n-Butyllithium (15.0 mL, 2.5 M in THF, 36.3 mmol) was added dropwise. The mixture was stirred at −60° C. for 0.5 h, and a solution of 4-fluoro-2-(2-methoxyethoxy)benzaldehyde (7.12 g, 35.9 mmol) in THF (20 mL) was added dropwise. The resulting mixture was stirred at 0° C. for 1 hour. Aqueous NH₄Cl solution was added slowly to quench the reaction. The product was extracted with EtOAc. The combined organic phases were washed with brine, dried over Na₂SO₄ and concentrated. The residue was then purified by column chromatography (petroleum ether/EtOAc 2:1) to give N,N-diethyl-2-[[4-fluoro-2-(2-methoxyethoxy)phenyl]-hydroxy-methyl]thiophene-3-carboxamide (9.60 g, 66% yield). LCMS ESI (+) m/z 714.2 (M+H).

Step B: Preparation of N,N-diethyl-2-[[4-fluoro-2-(2-methoxyethoxy)phenyl]methyl]thiophene-3-carboxamide: To a solution of N,N-diethyl-2-[[4-fluoro-2-(2-methoxyethoxy)phenyl]-hydroxy-methyl]thiophene-3-carboxamide (9.60 g, 25.2 mmol) in methanol (80 mL) was treated with 10% Pd/C (9.6 g) under hydrogen atmosphere at 50° C. for 2 days. The mixture was filtered through Celite® with methanol rinse (2×50 mL). The filtrate was concentrated under reduced pressure and purified by column chromatography (petroleum ether/EtOAc 5:1) to give N,N-diethyl-2-[[4-fluoro-2-(2-methoxyethoxy)phenyl]methyl]thiophene-3-carboxamide (7.20 g, 78% yield) as a yellow oil. 1H NMR (400 MHz, CD₃OD) δ: 7.13 (t, J=7.6 Hz, 1H), 7.70 (d, J=5.2 Hz, 1H), 6.86 (d, J=5.2 Hz, 1H), 6.56-6.60 (m, 2H), 4.08-4.12 (m, 4H), 3.77 (t, J=4.8 Hz, 2H), 3.51 (q, J=6.8 Hz, 2H), 3.14 (q, J=6.8 Hz, 2H), 1.21 (t, J=6.8 Hz, 3H), 1.02 (t, J=6.8 Hz, 3H).

Step C: Preparation of 7-(4-fluoro-2-(2-methoxyethoxy)phenyl)-6-(1-((2-(trimethylsilyl)ethoxy)methyl)-1H-pyrazol-4-yl)thieno[3,2-c]pyridin-4-ol: To a solution N,N-diethyl-2-[[4-fluoro-2-(2-methoxyethoxy)phenyl]methyl]thiophene-3-carboxamide (3.50 g, 9.58 mmol) in THF (40 mL) was added n-BuLi (2.5 M solution in hexanes, 8.40 mL, 21.1 mmol) dropwise at −78° C. The mixture was stirred at −65° C. for 1 hour. A solution of 1-(2-trimethylsilylethoxymethyl)pyrazole-4-carbonitrile (2.14 g, 9.58 mmol) in THF (10 mL) was then added, and the resulting mixture was stirred at ambient temperature for additional 2 hours. The reaction was quenched by the addition of water. 1 M aqueous HCl was added to adjust pH to 5. The mixture was diluted with water (60 mL) and extracted with EtOAc (80 mL×3). The combined organic extracts were washed with brine, dried over Na₂SO₄, filtered and concentrated under reduced pressure. The residue was purified by column chromatography (MeOH/DCM=1:100 to 1:60) to give 7-(4-fluoro-2-(2-methoxyethoxy)phenyl)-6-(1-((2-(trimethylsilyl)ethoxy)methyl)-1H-pyrazol-4-yl)thieno[3,2-c]pyridin-4-ol (3.1 g, 63% yield) as a yellow solid. LCMS ESI (+) m/z 516.2 (M+H).

Step D: Preparation of 7-(4-fluoro-2-(2-methoxyethoxy)phenyl)-6-(1H-pyrazol-4-yl)thieno[3,2-c]pyridin-4-ol: To a solution of 7-[4-fluoro-2-(2-methoxyethoxy)phenyl]-6-[1-(2-trimethylsilylethoxymethyl)pyrazol-4-yl]thieno[3,2-c]pyridin-4-ol (3.10 g, 6.02 mmol) in DCM (27 mL) was added TFA (9 mL). The mixture was stirred at ambient temperature for 2 hours. The reaction was concentrated under reduced pressure. The residue was dissolved in DCM (10 mL). Saturated sodium bicarbonate solution was added to adjust pH to 9 and the mixture was stirred for addition 0.5 h. The mixture was filtered and the filter cake was collected and dried in vacuo to give 7-(4-fluoro-2-(2-methoxyethoxy)phenyl)-6-(1H-pyrazol-4-yl)thieno[3,2-c]pyridin-4-ol (1.8 g, 78% yield) as an off-white solid which was used in the next step without further purification. LCMS ESI (+) m/z 386.1 (M+H).

Step E: Preparation of 4-chloro-7-(4-fluoro-2-(2-methoxyethoxy)phenyl)-6-(1H-pyrazol-4-yl)thieno[3,2-c]pyridine: To a solution of 7-[4-fluoro-2-(2-methoxyethoxy)phenyl]-6-(1H-pyrazol-4-yl)thieno[3,2-c]pyridin-4-ol (1.80 g, 4.67 mmol) in phosphorus oxychloride (25 mL) was added DMF (0.2 mL). The mixture stirred for 14 hours at 100° C. The mixture was concentrated under vacuum. The residue was diluted with DCM (15 mL) and poured into ice water (80 mL). Saturated sodium bicarbonate solution was added to adjust pH to 9. The product was extracted EtOAc (70 mL×3). The combined organic layers were dried over Na₂SO₄ and concentrated. The residue was purified by column chromatography (MeOH/DCM=1:100 to 1:70) to give 4-chloro-7-(4-fluoro-2-(2-methoxyethoxy)phenyl)-6-(1H-pyrazol-4-yl)thieno[3,2-c]pyridine (1.2 g, 64% yield) as a yellow solid. LCMS ESI (+) m/z 404.1 (M+H).

Step F: Preparation of tert-butyl 3-[4-[4-chloro-7-[4-fluoro-2-(2-methoxyethoxy)phenyl]thieno[3,2-c]pyridin-6-yl]pyrazol-1-yl]azetidine-1-carboxylate: A suspension of 4-chloro-7-[4-fluoro-2-(2-methoxyethoxy)phenyl]-6-(1H-pyrazol-4-yl)thieno[3,2-c]pyridine (70 mg, 0.17 mmol), tert-butyl 3-iodoazetidine-1-carboxylate (147 mg, 0.520 mmol) and cesium carbonate (282 mg, 0.867 mmol) in DMF (2 mL) was stirred at 80° C. for 2 hours. The mixture was diluted with water (30 mL), and the product was extracted with EtOAc (40 mL×3). The organic layers were washed with saturated brine solution, dried over Na₂SO₄ and concentrated. The crude was then purified by preparative TLC (MeOH/DCM=1/20) to give tert-butyl 3-[4-[4-chloro-7-[4-fluoro-2-(2-methoxyethoxy)phenyl]thieno[3,2-c]pyridin-6-yl]pyrazol-1-yl]azetidine-1-carboxylate (80 mg, 83% yield) as colorless oil. LCMS ESI (+) m/z 559.1 (M+H).

Step G: Preparation of 6-(1-(azetidin-3-yl)-1H-pyrazol-4-yl)-4-chloro-7-(4-fluoro-2-(2-methoxyethoxy)phenyl)thieno[3,2-c]pyridine: To a solution of tert-butyl 3-[4-[4-chloro-7-[4-fluoro-2-(2-methoxyethoxy)phenyl]thieno[3,2-c]pyridin-6-yl]pyrazol-1-yl]azetidine-1-carboxylate (80 mg, 0.14 mmol) in DCM (3 mL) was added TFA (1 mL). The mixture was stirred at ambient temperature for 1 hour. The mixture was concentrated to dryness to give crude 6-(1-(azetidin-3-yl)-1H-pyrazol-4-yl)-4-chloro-7-(4-fluoro-2-(2-methoxyethoxy)phenyl)thieno[3,2-c]pyridine (100 mg), which was used in the next step without further purification. LCMS ESI (+) m/z 459.1 (M+H).

Step H: Preparation of 1-[3-[4-[4-chloro-7-[4-fluoro-2-(2-methoxyethoxy)phenyl]thieno[3,2-c]pyridin-6-yl]pyrazol-1-yl]azetidin-1-yl]prop-2-en-1-one: To a solution of 6-[1-(azetidin-3-yl)pyrazol-4-yl]-4-chloro-7-[4-fluoro-2-(2-methoxyethoxy)phenyl]thieno[3,2-c]pyridine (62 mg, 0.14 mmol) and triethylamine (0.056 mL, 0.41 mmol) in DCM (3 mL) was added prop-2-enoyl prop-2-enoate (20 mg, 0.16 mmol). The mixture was stirred at −60° C. for 1 hour. The mixture was quenched with saturated sodium bicarbonate solution (2 mL) and diluted with water (10 mL). The product was extracted with EtOAc (20 mL×3). The combined organic layers were washed with brine (10 mL), dried over Na₂SO₄, filtered and concentrated. The residue was purified by preparative TLC (MeOH/DCM: 1/20) to give 1-[3-[4-[4-chloro-7-[4-fluoro-2-(2-methoxyethoxy)phenyl]thieno[3,2-c]pyridin-6-yl]pyrazol-1-yl]azetidin-1-yl]prop-2-en-1-one (60 mg, 87% yield). LCMS ESI (+) m/z 513.1 (M+H).

Step I: Preparation of tert-butyl 6-[7-[4-fluoro-2-(2-methoxyethoxy)phenyl]-6-[1-(1-prop-2-enoylazetidin-3-yl)pyrazol-4-yl]thieno[3,2-c]pyridin-4-yl]-3,4-dihydro-1H-isoquinoline-2-carboxylate: A suspension of 1-[3-[4-[4-chloro-7-[4-fluoro-2-(2-methoxyethoxy)phenyl]thieno[3,2-c]pyridin-6-yl]pyrazol-1-yl]azetidin-1-yl]prop-2-en-1-one (30 mg, 0.059 mmol), tert-butyl 6-(4,4,5,5-tetramethyl-1,3,2-dioxaborolan-2-yl)-3,4-dihydro-1H-isoquinoline-2-carboxylate (32 mg, 0.088 mmol), tetrakis(triphenylphosphine)palladium(0) (6.8 mg, 0.0059 mmol) and sodium carbonate (12 mg, 0.12 mmol) in 1,4-dioxane (1.2 mL) and water (0.3 mL) was stirred for 4 hours at 95° C. under N₂ atmosphere. The mixture was concentrated and purified by flash column chromatography (MeOH/DCM=1/20) to give tert-butyl 6-[7-[4-fluoro-2-(2-methoxyethoxy)phenyl]-6-[1-(1-prop-2-enoylazetidin-3-yl)pyrazol-4-yl]thieno[3,2-c]pyridin-4-yl]-3,4-dihydro-1H-isoquinoline-2-carboxylate (30 mg, 72% yield). LCMS ESI (+) m/z 710.3 (M+H).

Step J: Preparation of tert-butyl 6-[7-[4-fluoro-2-(2-methoxyethoxy)phenyl]-6-[1-(1-prop-2-enoylazetidin-3-yl)pyrazol-4-yl]thieno[3,2-c]pyridin-4-yl]-3,4-dihydro-1H-isoquinoline-2-carboxylate: To a solution of tert-butyl 6-[7-[4-fluoro-2-(2-methoxyethoxy)phenyl]-6-[1-(1-prop-2-enoylazetidin-3-yl)pyrazol-4-yl]thieno[3,2-c]pyridin-4-yl]-3,4-dihydro-1H-isoquinoline-2-carboxylate (25 mg, 0.035 mmol) in DCM (3 mL) was added TFA (1 mL). The mixture was stirred at ambient temperature for 1 hour. The mixture was concentrated under reduced pressure, and the residue was purified by reverse phase preparative-HPLC to give 1-[3-[4-[7-[4-fluoro-2-(2-methoxyethoxy)phenyl]-4-(1,2,3,4-tetrahydroisoquinolin-6-yl)thieno[3,2-c]pyridin-6-yl]pyrazol-1-yl]azetidin-1-yl]prop-2-en-1-one (11 mg, 50% yield) as a trifluoroacetic acid salt. LCMS ESI (+) m/z 610.2 (M+H). ¹H NMR (400 MHz, CD₃OD) δ 7.85-7.87 (m, 2H), 7.72-7.75 (m, 2H), 7.63-7.65 (m, 1H), 7.53 (d, J=4.0 Hz, 1H), 7.48 (d, J=8.0 Hz, 1H), 7.32-7.34 (m, 1H), 7.07 (dd, J=11.2, 2.0 Hz, 1H), 6.90-6.95 (m, 1H), 6.25-6.40 (m, 2H), 5.78 (dd, J=10.0, 2.0 Hz, 1H), 5.20-5.27 (m, 1H), 4.74 (t, J=8.8 Hz, 1H), 4.47-4.57 (m, 4H), 4.30 (dd, J=10.8, 4.8 Hz, 1H), 3.97-4.09 (m, 2H), 3.61 (t, J=6.0 Hz, 2H), 3.42 (t, J=4.4 Hz, 2H), 3.27-3.28 (m, 2H), 3.05 (s, 3H).

### Synthetic Example 8: Synthesis of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (Compound 58)

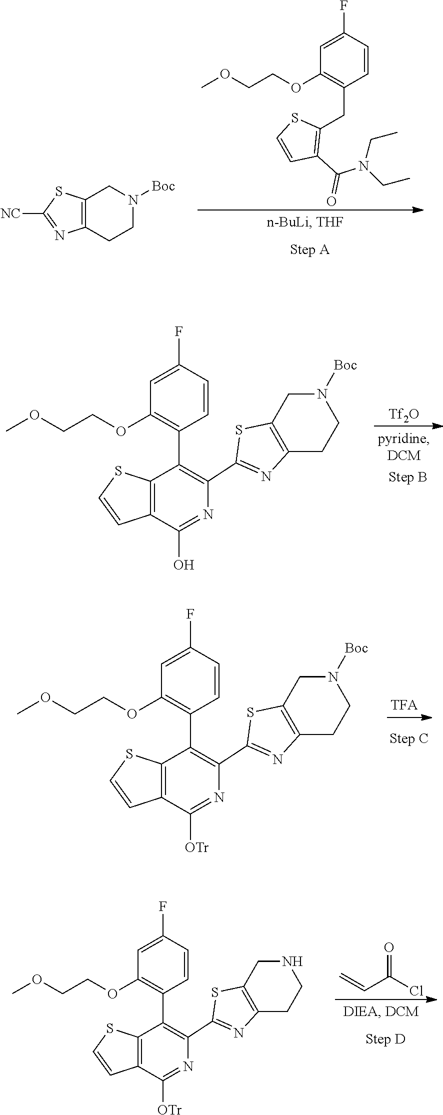

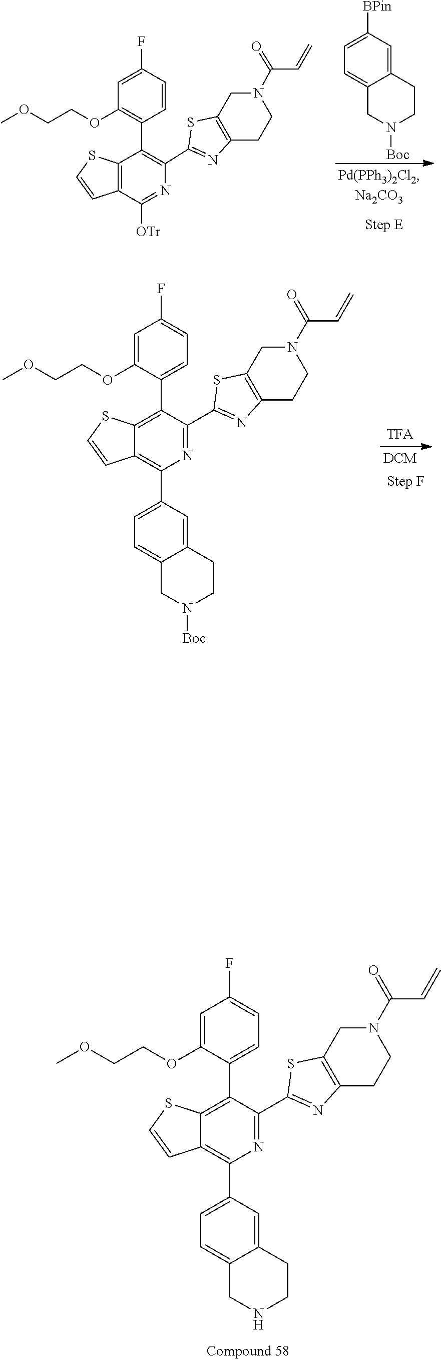

Step A: Preparation of tert-butyl 2-[7-[4-fluoro-2-(2-methoxyethoxy)phenyl]-4-hydroxy-thieno[3,2-c]pyridin-6-yl]-6,7-dihydro-4H-thiazolo[5,4-c]pyridine-5-carboxylate: To a solution of N,N-diethyl-2-[[4-fluoro-2-(2-methoxyethoxy)phenyl]methyl]thiophene-3-carboxamide (10.0 g, 27.4 mmol) in THF (100 mL) was added n-butyllithium (11.0 mL, 2.5 M solution in hexanes, 28.1 mmol) at −60° C. After the addition, the mixture was allowed to warm to 0° C. and stirred at this temperature for 15 min. tert-Butyl 2-cyano-6,7-dihydro-4H-thiazolo[5,4-c]pyridine-5-carboxylate (6.00 g, 22.6 mmol) was then added. The resulting mixture was stirred at 25° C. for 3 h. The mixture was poured into water. 1 N aqueous HCl was added to adjust the pH to 5. The mixture was extracted with EtOAc, dried over anhydrous Na₂SO₄, filtered and concentrated. The residue was purified by column chromatography (50 to 100% EtOAc in petroleum ether) to give tert-butyl 2-[7-[4-fluoro-2-(2-methoxyethoxy)phenyl]-4-hydroxy-thieno[3,2-c]pyridin-6-yl]-6,7-dihydro-4H-thiazolo[5,4-c]pyridine-5-carboxylate (9.60 g, about 50% purity). LCMS ESI (+) m/z 558.2 (M+H).

Step B: Preparation of tert-butyl 2-[7-[4-fluoro-2-(2-methoxyethoxy)phenyl]-4-(trifluoromethylsulfonyloxy)thieno[3,2-c]pyridin-6-yl]-6,7-dihydro-4H-thiazolo[5,4-c]pyridine-5-carboxylate: To a solution of tert-butyl 2-[7-[4-fluoro-2-(2-methoxyethoxy)phenyl]-4-hydroxy-thieno[3,2-c]pyridin-6-yl]-6,7-dihydro-4H-thiazolo[5,4-c]pyridine-5-carboxylate (9.60 g, 10.3 mmol) and pyridine (8.40 mL, 103 mmol) in DCM (100 mL) was added trifluoromethanesulfonic anhydride (3.50 mL, 20.7 mmol). The mixture was stirred at 25° C. for 1 h. The mixture was concentrated and diluted with EtOAc, washed with brine, dried over anhydrous Na₂SO₄ and concentrated. The residue was purified by column chromatography (EtOAc/petroleum ether: 1/3) to give tert-butyl 2-[7-[4-fluoro-2-(2-methoxyethoxy)phenyl]-4-(trifluoromethylsulfonyloxy)thieno[3,2-c]pyridin-6-yl]-6,7-dihydro-4H-thiazolo[5,4-c]pyridine-5-carboxylate (5.30 g, 74% yield). ¹H NMR (400 MHz, CDCl₃) δ 7.66 (d, J=5.6 Hz, 1H), 7.53 (d, J=5.6 Hz, 1H), 7.28-7.30 (m, 1H), 6.82 (dt, J=8.3, 2.3 Hz, 1H), 6.73 (dd, J=10.7, 2.4 Hz, 1H), 4.50-4.71 (m, 2H), 3.97-4.02 (m, 1H), 3.75-3.80 (m, 1H), 3.55-3.74 (m, 2H), 3.22-3.33 (m, 2H), 3.04 (s, 3H), 2.60 (br, 2H), 1.47 (s, 9H).

Step C: Preparation of [7-[4-fluoro-2-(2-methoxyethoxy)phenyl]-6-(4,5,6,7-tetrahydrothiazolo[5,4-c]pyridin-2-yl)thieno[3,2-c]pyridin-4-yl]trifluoromethanesulfonate: A solution of tert-butyl 2-[7-[4-fluoro-2-(2-methoxyethoxy)phenyl]-4-(trifluoromethylsulfonyloxy)thieno[3,2-c]pyridin-6-yl]-6,7-dihydro-4H-thiazolo[5,4-c]pyridine-5-carboxylate (3.00 g, 4.35 mmol) and trifluoroacetic acid (10.0 mL, 130 mmol) in DCM (30 mL) was stirred at 25° C. for 1 hour. The resulting mixture was evaporated to dryness to afford [7-[4-fluoro-2-(2-methoxyethoxy)phenyl]-6-(4,5,6,7-tetrahydrothiazolo[5,4-c]pyridin-2-yl)thieno[3,2-c]pyridin-4-yl]trifluoromethanesulfonate (3.20 g, 100% yield) as a yellow solid. LCMS ESI (+) m/z 590.0 (M+H).

Step D: Preparation of [7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]trifluoromethanesulfonate: To a solution of [7-[4-fluoro-2-(2-methoxyethoxy)phenyl]-6-(4,5,6,7-tetrahydrothiazolo[5,4-c]pyridin-2-yl)thieno[3,2-c]pyridin-4-yl]trifluoromethanesulfonate (3.20 g, 4.34 mmol) and DIEA (7.70 mL, 43.4 mmol) in DCM (30 mL) was added acryloyl chloride (0.53 mL, 6.5 mmol) under nitrogen, and the resulting mixture was stirred at −60° C. for 1 hour. The reaction mixture was poured into saturated NaHCO₃ (50 mL), and the mixture extracted with DCM (50 mL×4). The organic layer was washed with water (50 mL), brine (50 mL), then dried over anhydrous sodium sulfate, filtered and concentrated to give [7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]trifluoromethanesulfonate (2.95 g, 95% yield) as a yellow solid.

Step E: Preparation of 4-chloro-7-(4-fluoro-2-(2-methoxyethoxy)phenyl)-6-(1H-pyrazol-4-yl)thieno[3,2-c]pyridine: To a solution of [7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]trifluoromethanesulfonate (600 mg, 0.932 mmol), tert-butyl 6-(4,4,5,5-tetramethyl-1,3,2-dioxaborolan-2-yl)-3,4-dihydro-1H-isoquinoline-2-carboxylate (502 mg, 1.40 mmol) and Na₂CO₃ (198 mg, 1.86 mmol) in 1,4-dioxane (5 mL) and water (0.50 mL) was added Pd(PPh₃)₂Cl₂ (16 mg, 0.023 mmol) under nitrogen. The resulting mixture was stirred at 100° C. for 2 hours. The solvent was removed under reduced pressure, and the residue was purified by column chromatography on silica gel (EtOAc/PE: 30 to 100% EtOAc) to afford tert-butyl 6-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (359 mg, 53% yield) as a yellow oil. LCMS ESI (+) m/z 727.4 (M+H).

Step F: Preparation of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one: To a solution of tert-butyl 6-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (359 mg, 0.494 mmol) in DCM (5 mL) was added trifluoroacetic acid (1.70 mL, 22.1 mmol). The resulting solution was stirred at 25° C. for 1 hour. The mixture was purified by reverse phase preparative-HPLC to give 1-[2-[7-[4-fluoro-2-(2-methoxyethoxy)phenyl]-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (323 mg, 104% yield) as a bis trifluoroacetic acid salt. LCMS ESI (+) m/z 627.3 (M+H). 1H NMR (400 MHz, CD₃OD) δ 7.87-7.92 (m, 2H), 7.81 (d, J=5.6 Hz, 1H), 7.71 (d, J=5.6 Hz, 1H), 7.45 (d, J=7.9 Hz, 1H), 7.35 (dd, J=8.2, 6.7 Hz, 1H), 6.93 (d, J=11.0 Hz, 1H), 6.73-6.88 (m, 2H), 6.20-6.28 (m, 1H), 5.75-5.82 (m, 1H), 4.50 (s, 2H), 4.00-4.07 (m, 1H), 3.82-3.95 (m, 3H), 3.61 (t, J=6.3 Hz, 2H), 3.00 (s, 3H), 2.66-2.75 (m, 2H).

### Synthetic Example 9: Alternate synthesis of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (Compound 58)

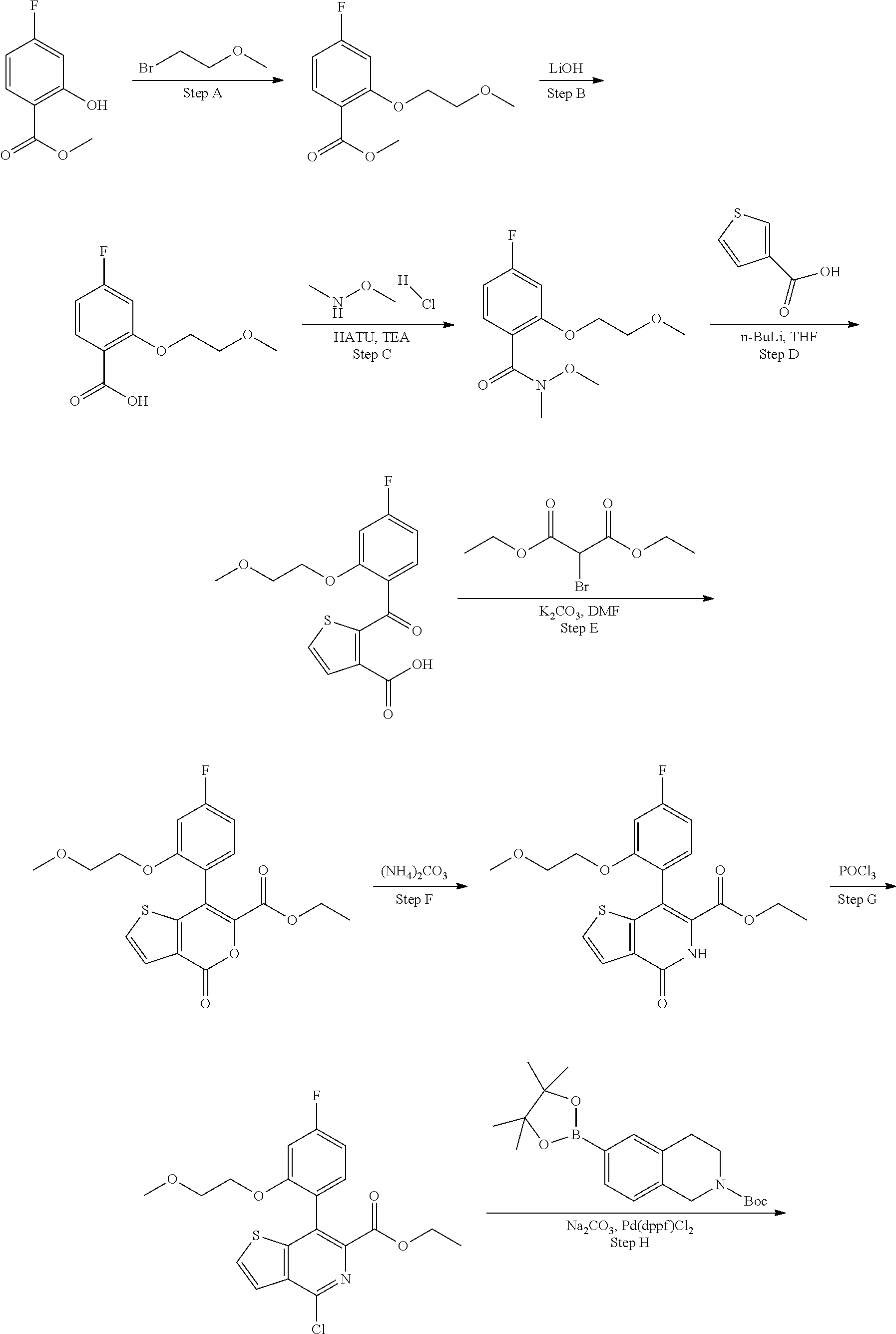

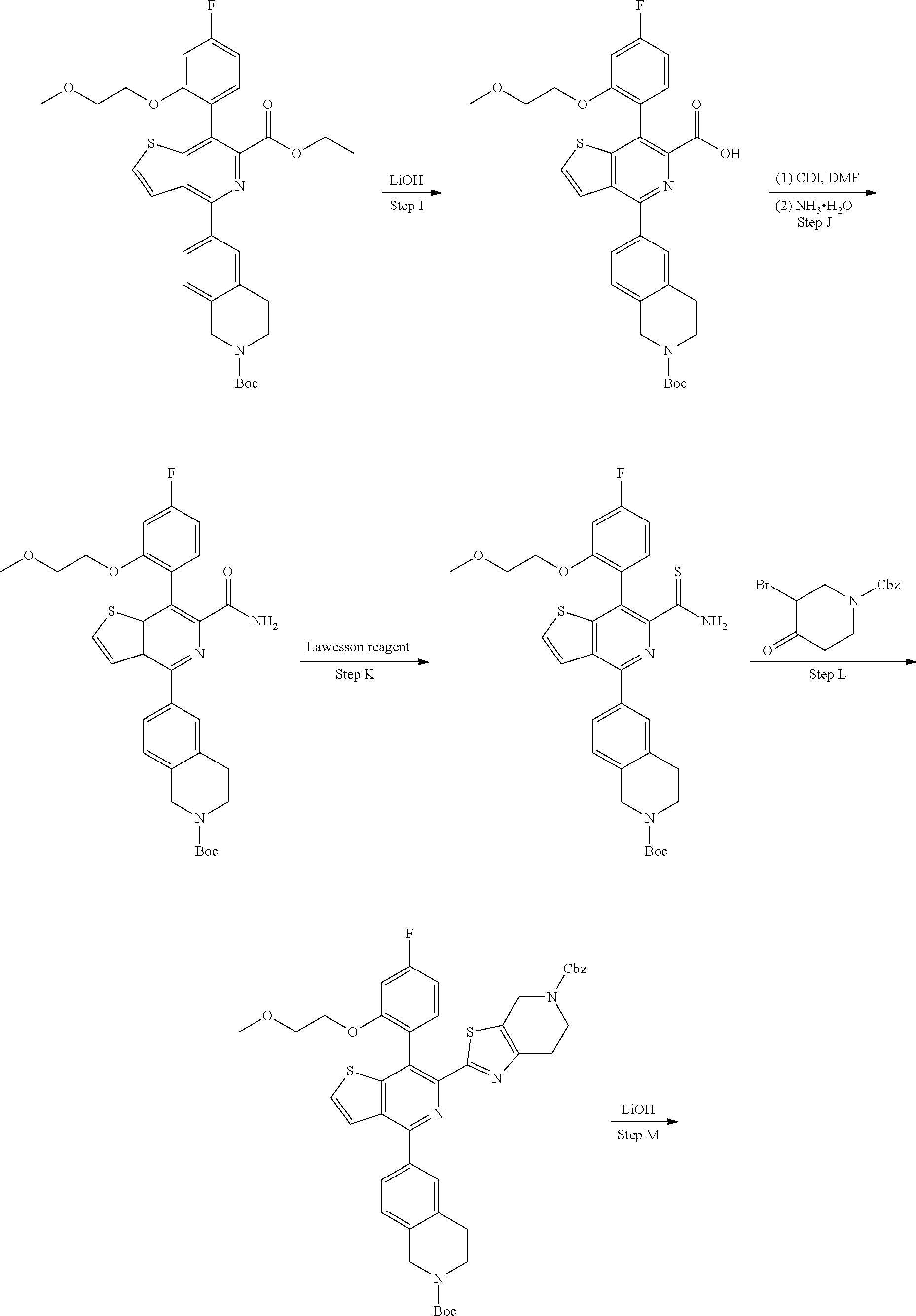

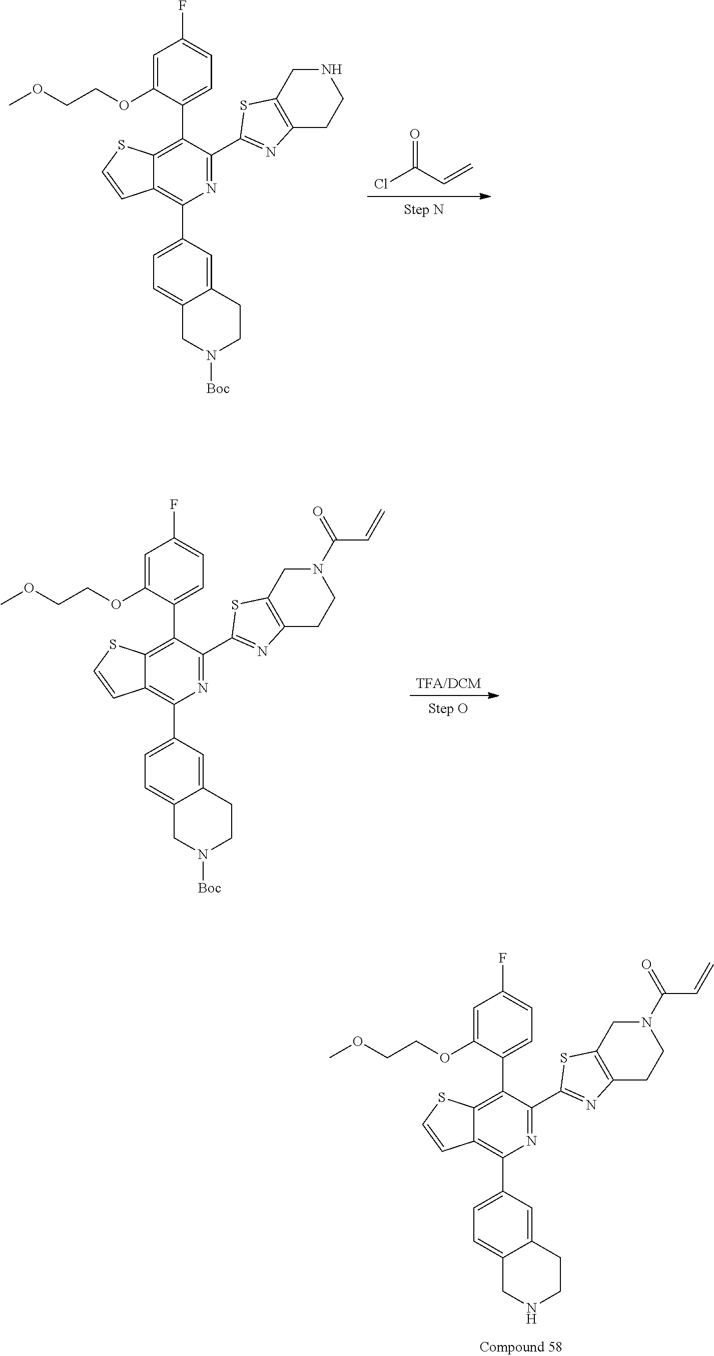

Step A: Preparation of methyl 4-fluoro-2-(2-methoxyethoxy)benzoate: To a solution of methyl 4-fluoro-2-hydroxy-benzoate (25.0 g, 147 mmol) in MeCN (500 mL) was added 1-bromo-2-methoxy-ethane (24.5 g, 176 mmol) and cesium carbonate (97.4 g, 220 mmol). The mixture was stirred at 70° C. for 16 h under Ar. The mixture was filtered, and the filtrate was concentrated under vacuum. The residue was diluted with water (200 mL) and extracted with ethyl acetate (100 mL×3). The organic layers were washed with saturated brine (100 mL), dried over Na₂SO₄ and concentrated in vacuum to give methyl 4-fluoro-2-(2-methoxyethoxy)benzoate (32.6 g, 97% yield) as a yellow oil. LCMS ESI (+) m/z 229.2 (M+H).

Step B: Preparation of 4-fluoro-2-(2-methoxyethoxy)benzoic acid: To a solution of methyl 4-fluoro-2-(2-methoxyethoxy)benzoate (16.3 g, 71.4 mmol) in THF (82 mL), methanol (27 mL) and water (27 mL) was added lithium hydroxide monohydrate (8.55 g, 357 mmol). The mixture was stirred at 25° C. for 6 h. The reaction was concentrated to remove THF and methanol, then diluted with water (150 mL). Aqueous HCl (3 N) was added to adjust the pH to 2. The mixture was extracted with ethyl acetate (70 mL×3). The combined organic layers were washed with saturated brine (70 ml), dried over Na₂SO₄ and concentrated in vacuum to give 4-fluoro-2-(2-methoxyethoxy)benzoic acid (14.8 g, 97% yield) as a white solid. ¹H NMR (400 MHz, CDCl₃) δ 8.18 (dd, J=8.8, 6.8 Hz, 1H), 6.80-6.91 (m, 1H), 6.76 (dd, J=10.0, 2.3 Hz, 1H), 4.34 (t, J=4.6 Hz, 2H), 3.82 (t, J=4.6 Hz, 2H), 3.46 (s, 3H), 2.10 (s, 1H).

Step C: Preparation of 4-fluoro-N-methoxy-2-(2-methoxyethoxy)-N-methyl-benzamide: To a solution of 4-fluoro-2-(2-methoxyethoxy)benzoic acid (10.0 g, 46.7 mmol), N,O-dimethylhydroxylamine hydrochloride (5.47 g, 56.0 mmol) and triethylamine (20.0 mL, 140 mmol) in DCM (150 mL) was added HATU (23.1 g, 60.7 mmol). The mixture was stirred at 25° C. for 6 h. The mixture was poured into water and the product was extracted with DCM. The organics were washed with brine, dried over anhydrous Na₂SO₄, filtered and concentrated. The residue was purified by flash chromatography on silica gel (EtOAc) to give 4-fluoro-N-methoxy-2-(2-methoxyethoxy)-N-methyl-benzamide (10.8 g, 89.9% yield). ¹H NMR (400 MHz, CDCl₃) δ 7.23-7.34 (m, 1H), 6.61-6.76 (m, 2H), 4.13 (t, J=4.8 Hz, 2H), 3.73 (t, J=4.8 Hz, 2H), 3.41 (s, 3H), 2.80 (s, 6H).

Step D: Preparation of 2-[4-fluoro-2-(2-methoxyethoxy)benzoyl]thiophene-3-carboxylic acid: To a solution of thiophene-3-carboxylic acid (5.92 g, 46.2 mmol) in THF (130 mL) was added n-butyllithium (37.0 mL, 2.5 M solution in hexanes, 92.4 mmol) dropwise at −60° C. under Ar. The mixture was stirred at −60° C. for 0.5 h under Ar, then 4-fluoro-N-methoxy-2-(2-methoxyethoxy)-N-methyl-benzamide (10.8 g, 42.0 mmol) was added at −60° C. The temperature was allowed to warm to 0° C., and the resulting mixture was stirred at 0° C. for additional 1 h. The mixture was poured into water. 1 N aqueous HCl was added to adjust the pH to 5. The mixture was concentrated to remove THF and diluted with water. The solid was filtered, washed with water and dried in vacuo to give crude 2-[4-fluoro-2-(2-methoxyethoxy)benzoyl]thiophene-3-carboxylic acid (13.0 g, 57% yield), LCMS ESI (+) m/z 325.1 (M+H).

Step E: Preparation of ethyl 7-[4-fluoro-2-(2-methoxyethoxy)phenyl]-4-oxo-thieno[3,2-c]pyran-6-carboxylate: To a solution of 2-[4-fluoro-2-(2-methoxyethoxy)benzoyl]thiophene-3-carboxylic acid (2.15 g, 6.63 mmol) and diethyl 2-bromopropanedioate (1.90 g, 7.96 mmol) in DMF (25 mL) was added potassium carbonate (2.29 g, 16.6 mmol). The mixture was stirred at 25° C. for 76 h. The mixture was poured into water and the product was extracted with EtOAc. The organics were washed with brine, dried over anhydrous Na₂SO₄, concentrated and purified by column chromatography on silica gel (EtOAc/petroleum ether=1/3) to give ethyl 7-[4-fluoro-2-(2-methoxyethoxy)phenyl]-4-oxo-thieno[3,2-c]pyran-6-carboxylate (2.10 g, 81% yield). LCMS ESI (+) m/z 393.1 (M+H).

Step F: Preparation of ethyl 7-[4-fluoro-2-(2-methoxyethoxy)phenyl]-4-oxo-5H-thieno[3,2-c]pyridine-6-carboxylate: To a mixture of ethyl 7-[4-fluoro-2-(2-methoxyethoxy)phenyl]-4-oxo-thieno[3,2-c]pyran-6-carboxylate (1.80 g, 4.59 mmol) and AcOH (50 mL) under argon was added (NH₄)₂CO₃ (6.61 g, 68.8 mmol) by portions. The mixture was stirred at 95° C. for 14 h. The mixture was allowed to cool to ambient temperature, and precipitate was collected by filtration. The filter-cake was washed with water and MeOH, and dried in vacuo to give ethyl 7-[4-fluoro-2-(2-methoxyethoxy)phenyl]-4-oxo-5H-thieno[3,2-c]pyridine-6-carboxylate (1.26 g, 70% yield). LCMS ESI (+) m/z 392.1 (M+H).

Step G: Preparation of ethyl 4-chloro-7-[4-fluoro-2-(2-methoxyethoxy)phenyl]thieno[3,2-c]pyridine-6-carboxylate: A suspension of ethyl 7-[4-fluoro-2-(2-methoxyethoxy)phenyl]-4-oxo-5H-thieno[3,2-c]pyridine-6-carboxylate (3.00 g, 5.37 mmol) in phosphorus oxychloride (10.0 mL, 107 mmol) was stirred at 95° C. for 3 h. The mixture was concentrated in vacuum and diluted with DCM. The mixture was poured into aqueous NaHCO₃ solution and the product was extracted with EtOAc. The organic layers were washed with brine, dried over anhydrous Na₂SO₄, filtered, concentrated and purified by flash chromatography on silica gel (EtOAc/petroleum ether=1/6) to give ethyl 4-chloro-7-[4-fluoro-2-(2-methoxyethoxy)phenyl]thieno[3,2-c]pyridine-6-carboxylate (1.09 g, 49% yield). LCMS ESI (+) m/z 410.1 (M+H).

Step H: Preparation of ethyl 4-(2-tert-butoxycarbonyl-3,4-dihydro-1H-isoquinolin-6-yl)-7-[4-fluoro-2-(2-methoxyethoxy)phenyl]thieno[3,2-c]pyridine-6-carboxylate: To a solution of tert-butyl 6-(4,4,5,5-tetramethyl-1,3,2-dioxaborolan-2-yl)-3,4-dihydro-1H-isoquinoline-2-carboxylate (1.48 g, 4.12 mmol), ethyl 4-chloro-7-[4-fluoro-2-(2-methoxyethoxy)phenyl]thieno[3,2-c]pyridine-6-carboxylate (1.30 g, 3.17 mmol), and Na₂CO₃ (0.841 g, 7.93 mmol) in 1,4-dioxane (20 mL) and water (2 mL) was added Pd(dppf)Cl₂ (220 mg, 0.300 mmol). The mixture was stirred at 95° C. for 16 h under Ar. The mixture was poured into water and the product was extracted with EtOAc. The combined organic layers were dried over anhydrous Na₂SO₄, concentrated and purified by chromatography on silica gel (EtOAc/petroleum ether=1/3) to give ethyl 4-(2-tert-butoxycarbonyl-3,4-dihydro-1H-isoquinolin-6-yl)-7-[4-fluoro-2-(2-methoxyethoxy)phenyl]thieno[3,2-c]pyridine-6-carboxylate (1.20 g, 62% yield). LCMS ESI (+) m/z 607.3 (M+H).

Step I: Preparation of 4-(2-tert-butoxycarbonyl-3,4-dihydro-1H-isoquinolin-6-yl)-7-[4-fluoro-2-(2-methoxyethoxy)phenyl]thieno[3,2-c]pyridine-6-carboxylic acid: To a solution of ethyl 4-(2-tert-butoxycarbonyl-3,4-dihydro-1H-isoquinolin-6-yl)-7-[4-fluoro-2-(2-methoxyethoxy)phenyl]thieno[3,2-c]pyridine-6-carboxylate (800 mg, 1.32 mmol) in 3:1:1 mixture of tetrahydrofuran, water and methanol (20.0 ml) was added LiOH·H₂O (554 mg, 13.2 mmol) at room temperature. The reaction mixture was stirred at room temperature for 3 hours and then the solvents evaporated. The residue was dissolved in water. The pH was adjusted to 3 with 1N HCl. The mixture was extracted with dichloromethane. The combined organic layers were washed water and brine, dried over anhydrous sodium sulphate, filtered and concentrated under reduced pressure to give 4-(2-tert-butoxycarbonyl-3,4-dihydro-1H-isoquinolin-6-yl)-7-[4-fluoro-2-(2-methoxyethoxy)phenyl]thieno[3,2-c]pyridine-6-carboxylic acid (605 mg, 79% yield) LCMS ESI (+) m/z 579.2 (M+H).

Step J: Preparation of tert-butyl 6-[6-carbamoyl-7-[4-fluoro-2-(2-methoxyethoxy)phenyl]thieno[3,2-c]pyridin-4-yl]-3,4-dihydro-1H-isoquinoline-2-carboxylate: To a solution of 4-(2-tert-butoxycarbonyl-3,4-dihydro-1H-isoquinolin-6-yl)-7-[4-fluoro-2-(2-methoxyethoxy)phenyl]thieno[3,2-c]pyridine-6-carboxylic acid (699 mg, 1.21 mmol) in DMF (10 mL) was added N,N′-carbonyldiimidazole (294 mg, 1.81 mmol) at 0° C. The reaction was stirred at 24° C. for 40 minutes. The mixture was added into stirred ammonium hydroxide (30 mL) dropwise. The resulting mixture was stirred at ambient temperature for 1 h. The mixture was diluted with water (80 mL) and extracted with EtOAc (50 mL×3). The combined organic layers were washed with saturated brine solution (100 mL). The organic layers were then separated and dried (MgSO₄) before concentration to dryness. The crude was then purified by column chromatography eluting with 30% EtOAc in hexanes to give tert-butyl 6-[6-carbamoyl-7-[4-fluoro-2-(2-methoxyethoxy)phenyl]thieno[3,2-c]pyridin-4-yl]-3,4-dihydro-1H-isoquinoline-2-carboxylate (480 mg, 69% yield) as a white solid. LCMS ESI (+) m/z 578.2 (M+H).

Step K: Preparation of tert-butyl 6-[6-carbamothioyl-7-[4-fluoro-2-(2-methoxyethoxy)phenyl]thieno[3,2-c]pyridin-4-yl]-3,4-dihydro-1H-isoquinoline-2-carboxylate: To a solution tert-butyl 6-[6-carbamoyl-7-[4-fluoro-2-(2-methoxyethoxy)phenyl]thieno[3,2-c]pyridin-4-yl]-3,4-dihydro-1H-isoquinoline-2-carboxylate (480 mg, 0.831 mmol) in THF (10 mL) was added Lawesson reagent (371 mg, 0.915 mmol) under Ar. The reaction was stirred at 60° C. for 3 hours. The reaction was concentrated to dryness and the residue was taken up in EtOAc (10 mL). The organic layers were washed with saturated brine solution and dried (MgSO₄) before concentration to dryness. The crude was then purified by flash column chromatography eluting with 30% EtOAc in hexanes to give tert-butyl 6-[6-carbamothioyl-7-[4-fluoro-2-(2-methoxyethoxy)phenyl]thieno[3,2-c]pyridin-4-yl]-3,4-dihydro-1H-isoquinoline-2-carboxylate (319 mg, 65% yield) as a white solid. LCMS ESI (+) m/z 594.2 (M+H).

Step L: Preparation of benzyl 2-[4-(2-tert-butoxycarbonyl-3,4-dihydro-1H-isoquinolin-6-yl)-7-[4-fluoro-2-(2-methoxyethoxy)phenyl]thieno[3,2-c]pyridin-6-yl]-6,7-dihydro-4H-thiazolo[5,4-c]pyridine-5-carboxylate: To a solution of tert-butyl 6-[6-carbamothioyl-7-[4-fluoro-2-(2-methoxyethoxy)phenyl]thieno[3,2-c]pyridin-4-yl]-3,4-dihydro-1H-isoquinoline-2-carboxylate (80 mg, 0.14 mmol) in ethanol (3 mL) was added benzyl 3-bromo-4-oxo-piperidine-1-carboxylate (59 mg, 0.19 mmol) under Ar. The mixture was stirred at 75° C. for 3 hours. The reaction mixture was concentrated to dryness and diluted with EtOAc (5 ml). The organics were washed with saturated brine solution, dried (MgSO₄) and concentrated. The residue was then purified by flash column chromatography eluting with 50% EtOAc in hexanes to give benzyl 2-[4-(2-tert-butoxycarbonyl-3,4-dihydro-1H-isoquinolin-6-yl)-7-[4-fluoro-2-(2-methoxyethoxy)phenyl]thieno[3,2-c]pyridin-6-yl]-6,7-dihydro-4H-thiazolo[5,4-c]pyridine-5-carboxylate (91 mg, 84% yield) as a light yellow solid.

Step M: Preparation of tert-butyl 6-[7-[4-fluoro-2-(2-methoxyethoxy)phenyl]-6-(4,5,6,7-tetrahydrothiazolo[5,4-c]pyridin-2-yl)thieno[3,2-c]pyridin-4-yl]-3,4-dihydro-1H-isoquinoline-2-carboxylate: To a solution of benzyl 2-[4-(2-tert-butoxycarbonyl-3,4-dihydro-1H-isoquinolin-6-yl)-7-[4-fluoro-2-(2-methoxyethoxy)phenyl]thieno[3,2-c]pyridin-6-yl]-6,7-dihydro-4H-thiazolo[5,4-c]pyridine-5-carboxylate (70 mg, 0.087 mmol) in 1,4-dioxane (1 mL) and water (1 mL) was added lithium hydroxide monohydrate (100 mg, 4.18 mmol) under Ar. The mixture was stirred at 100° C. for 40 hours. The reaction was diluted with water and extracted with EtOAc. The organics were washed with saturated brine solution, dried (MgSO₄) and concentrated to dryness. The residue was then purified by flash column chromatography eluting with 10% MeOH in DCM to give tert-butyl 6-[7-[4-fluoro-2-(2-methoxyethoxy) phenyl]-6-(4,5,6,7-tetrahydrothiazolo[5,4-c]pyridin-2-yl)thieno[3,2-c]pyridin-4-yl]-3,4-dihydro-1H-isoquinoline-2-carboxylate (35 mg, 60% yield) as a colorless oil. LCMS ESI (+) m/z 673.2 (M+H).

Step N: Preparation of tert-butyl 6-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate: To a mixture of tert-butyl 6-[7-[4-fluoro-2-(2-methoxyethoxy)phenyl]-6-(4,5,6,7-tetrahydrothiazolo[5,4-c]pyridin-2-yl)thieno[3,2-c]pyridin-4-yl]-3,4-dihydro-1H-isoquinoline-2-carboxylate (35 mg, 0.052 mmol) and N,N-diisopropylethylamine (0.018 mL, 0.10 mmol) in DCM (2 mL) was added acryloyl chloride (0.0042 mL, 0.052 mmol) dropwise at −60° C. The reaction was then allowed to warm to 0° C. and stirred for 1 hour. The reaction was quenched by the addition of saturated NaHCO₃ aqueous solution. The product was extracted with EtOAc and washed with saturated brine solution. The organic layers were then separated, dried (MgSO₄) and concentrated to dryness. The residue was purified by preparative TLC (30% EtOAc in hexane) to give tert-butyl 6-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (22 mg, 58% yield). LCMS ESI (+) m/z 727.3 (M+H).

Step O: Preparation of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one: The title compound was prepared as described in Step F of Synthetic Example 8. LCMS ESI (+) m/z 627.3 (M+H). 1H NMR (400 MHz, CD₃OD) δ 7.87-7.92 (m, 2H), 7.81 (d, J=5.6 Hz, 1H), 7.71 (d, J=5.6 Hz, 1H), 7.45 (d, J=7.9 Hz, 1H), 7.35 (dd, J=8.2, 6.7 Hz, 1H), 6.93 (d, J=11.0 Hz, 1H), 6.73-6.88 (m, 2H), 6.20-6.28 (m, 1H), 5.75-5.82 (m, 1H), 4.50 (s, 2H), 4.00-4.07 (m, 1H), 3.82-3.95 (m, 3H), 3.61 (t, J=6.3 Hz, 2H), 3.00 (s, 3H), 2.66-2.75 (m, 2H).

### Synthetic Example 10: Synthesis of 1-[3-[7-[4-fluoro-2-(2-methoxyethoxy)phenyl]-4-(1,2,3,4-tetrahydroisoquinolin-6-yl)thieno[3,2-c]pyridin-6-yl]-7,8-dihydro-5H-1,6-naphthyridin-6-yl]prop-2-en-1-one (Compound 62)

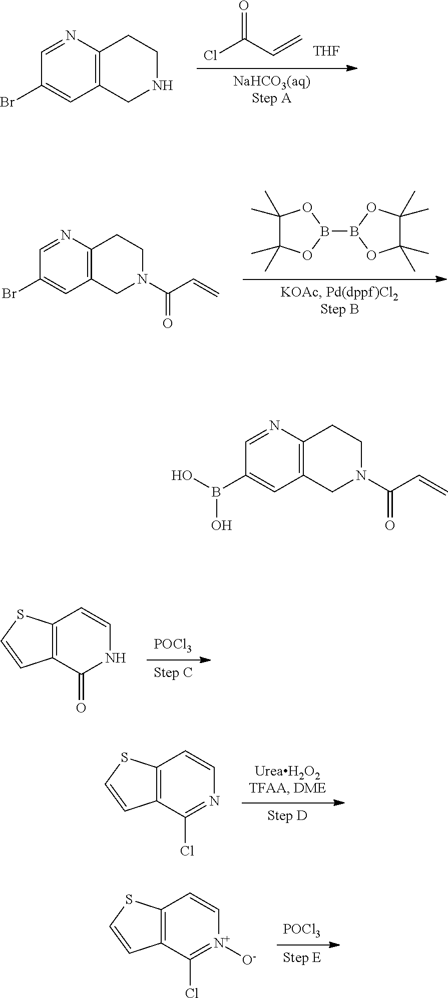

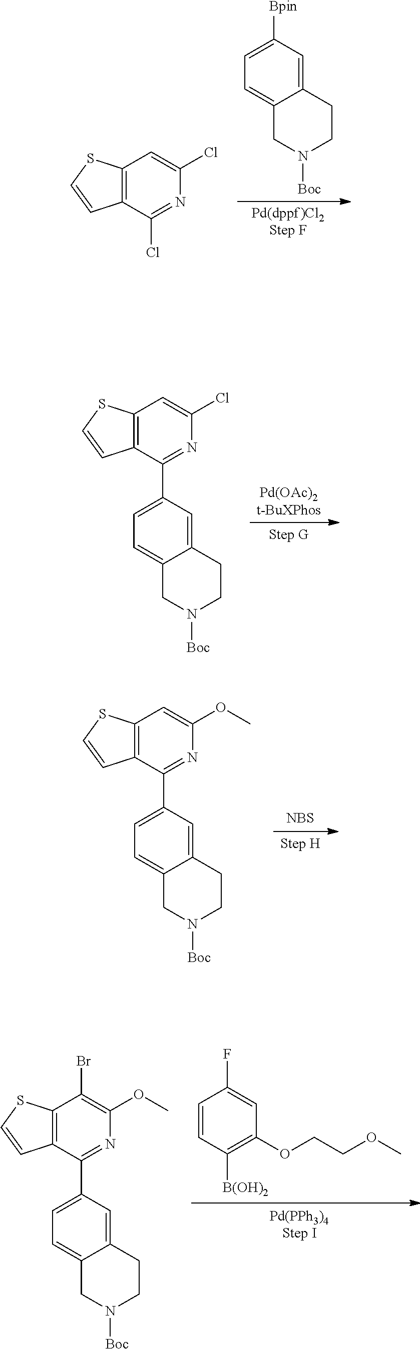

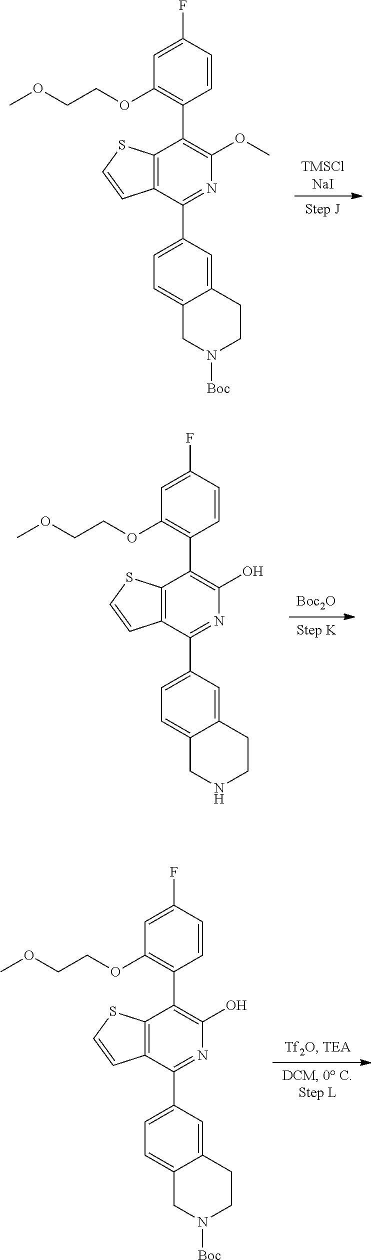

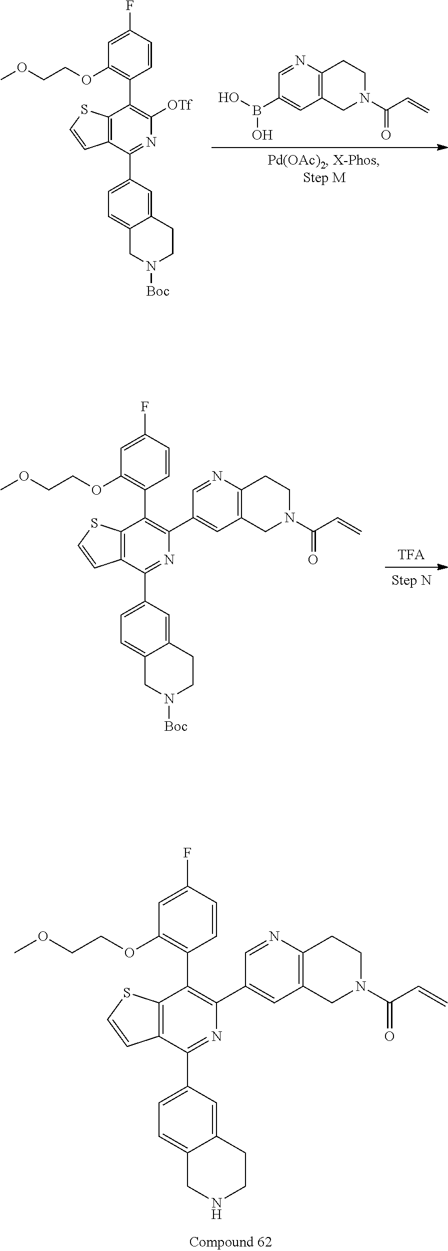

Step A: Preparation of 1-(3-bromo-7,8-dihydro-5H-1,6-naphthyridin-6-yl)prop-2-en-1-one: To a solution of 3-bromo-5,6,7,8-tetrahydro-1,6-naphthyridine dihydrochloride (200 mg, 0.699 mmol) in water (3 mL) was added sodium bicarbonate (300 mg, 3.57 mmol). The mixture was stirred at ambient temperature for 10 min, then acryloyl chloride (0.083 mL, 1.02 mmol) in THF (2 mL) was added dropwise at 0° C. The mixture was poured into NaHCO₃ aqueous solution. The mixture was extracted with EtOAc, washed with brine solution, dried over anhydrous Na₂SO₄, filtered and concentrated to dryness to give crude 1-(3-bromo-7,8-dihydro-5H-1,6-naphthyridin-6-yl)prop-2-en-1-one (182 mg, 97% yield) which was used in the next step directly. LCMS ESI (+) m/z 266, 268 (M+H).

Step B: Preparation of (6-prop-2-enoyl-7,8-dihydro-5H-1,6-naphthyridin-3-yl)boronic acid: To a solution of 1-(3-bromo-7,8-dihydro-5H-1,6-naphthyridin-6-yl)prop-2-en-1-one (90 mg, 0.34 mmol), bis(pinacolato)diboron (94 mg, 0.37 mmol) and potassium acetate (74 mg, 0.76 mmol) in 1,4-dioxane (5 mL) was added 1,1′-bis(diphenylphosphino)ferrocenedichloro palladium(II) dichloromethane complex (21 mg, 0.028 mmol). The mixture was stirred at 100° C. for 6 h under Ar. The mixture was filtered and the filtrate was concentrated to dryness to give (6-prop-2-enoyl-7,8-dihydro-5H-1,6-naphthyridin-3-yl)boronic acid (170 mg) which was used in the next step directly.

Step C: Preparation of 4-chlorothieno[3,2-c]pyridine: A mixture of 5H-thieno[3,2-c]pyridin-4-one (10.0 g, 66.1 mmol) in phosphorus oxychloride (100 mL) was stirred at 100° C. for 4 hours. The mixture was concentrated and poured into water. NaHCO₃ solution was added to adjust pH to 8-9. The product was extracted with EtOAc, washed by brine, dried over Na₂SO₄ and concentrated to give 4-chlorothieno[3,2-c]pyridine (10.0 g, 89% yield). LCMS: (ES+): m/z 170.0 [M−17].

Step D: Preparation of 4-chloro-5-oxido-thieno[3,2-c]pyridin-5-ium: To a stirred solution of trifluoroacetic anhydride (1.70 mL, 11.8 mmol) in 1,2-dimethoxyethane (5 mL) was added urea hydrogen peroxide (1.11 g, 11.8 mmol) by portions at 0° C. The mixture was stirred at 25° C. for 15 min. 4-Chlorothieno[3,2-c]pyridine (0.500 g, 2.95 mmol) was added to the above solution. The resulting mixture was stirred at 25° C. for 12 hours. Aqueous Na₂S₂O₃ solution was added to quench the reaction, and NaHCO₃ was added to adjust pH to 8-9. The product was extracted with EtOAc, dried over Na₂SO₄ and concentrated. The residue was purified by silica gel column (EtOAc to DCM:MeOH=20:1) to give 4-chloro-5-oxido-thieno[3,2-c]pyridin-5-ium (280 mg, 51% yield). LCMS ESI (+) m/z 186.1 (M+H).

Step E: Preparation of 4,6-dichlorothieno[3,2-c]pyridine: A mixture of 4-chloro-5-oxido-thieno[3,2-c]pyridin-5-ium (530 mg, 2.86 mmol) and phosphorus oxychloride (5.00 mL, 53.6 mmol) was stirred at 100° C. for 4 hours. The mixture was concentrated and poured into ice-water. Aqueous NaHCO₃ solution was added to adjust pH to 8-9. The product was extracted with EtOAc, washed by brine, dried over Na₂SO₄ and concentrated to give 4,6-dichlorothieno[3,2-c]pyridine (350 mg, 60% yield). LCMS ESI (+) m/z 205.1 (M+H).

Step F: Preparation of tert-butyl 6-(6-chlorothieno[3,2-c]pyridin-4-yl)-3,4-dihydro-1H-isoquinoline-2-carboxylate: A mixture of 4,6-dichlorothieno[3,2-c]pyridine (1.16 g, 5.68 mmol), tert-butyl 6-(4,4,5,5-tetramethyl-1,3,2-dioxaborolan-2-yl)-3,4-dihydro-1H-isoquinoline-2-carboxylate (2.65 g, 7.39 mmol), 1,1′-bis(diphenylphosphino)ferrocenedichloro palladium(II) dichloromethane complex (416 mg, 0.568 mmol) and sodium carbonate (1.20 g, 11.4 mmol) in 1,4-dioxane (20 mL)/water (2 mL) was stirred at 85° C. under N₂ for 8 hours. The mixture was concentrated and purified by silica gel column (petroleum ether:EA=10:1) to give tert-butyl 6-(6-chlorothieno[3,2-c]pyridin-4-yl)-3,4-dihydro-1H-isoquinoline-2-carboxylate (900 mg, 39% yield). LCMS ESI (+) m/z 401.1 (M+H).

Step G: Preparation of get tert-butyl 6-(6-methoxythieno[3,2-c]pyridin-4-yl)-3,4-dihydro-1H-isoquinoline-2-carboxylate: A mixture of tert-butyl 6-(6-chlorothieno[3,2-c]pyridin-4-yl)-3,4-dihydro-1H-isoquinoline-2-carboxylate (700 mg, 1.75 mmol), palladium(II) acetate (39 mg, 0.18 mmol), t-Bu XPhos (148 mg, 0.349 mmol) and palladium(II) acetate (39 mg, 0.18 mmol) in 1,4-dioxane (5 mL) and methanol (5 mL) was stirred at 80° C. under N₂ for 8 hours. The mixture was concentrated and purified by silica gel (petroleum ether:EtOAc=5:1) to give tert-butyl 6-(6-methoxythieno[3,2-c]pyridin-4-yl)-3,4-dihydro-1H-isoquinoline-2-carboxylate (640 mg, 92% yield). LCMS ESI (+) m/z 397.2 (M+H).

Step H: Preparation of tert-butyl 6-(7-bromo-6-methoxy-thieno[3,2-c]pyridin-4-yl)-3,4-dihydro-1H-isoquinoline-2-carboxylate: To a solution of tert-butyl 6-(6-methoxythieno[3,2-c]pyridin-4-yl)-3,4-dihydro-1H-isoquinoline-2-carboxylate (200 mg, 0.504 mmol) in DMF (2 mL) was added N-bromosuccinimide (90 mg, 0.50 mmol). The mixture was stirred for 2 hours. The mixture was diluted with EtOAc, washed by water and brine. The organics were dried over Na₂SO₄, filtered, concentrated and purified by silica gel column (petroleum ether:EtOAc=5:1) to give tert-butyl 6-(7-bromo-6-methoxy-thieno[3,2-c]pyridin-4-yl)-3,4-dihydro-1H-isoquinoline-2-carboxylate (238 mg, 99% yield). LCMS ESI (+) m/z 475.0 (M+H).

Step I: Preparation of tert-butyl 6-[7-[4-fluoro-2-(2-methoxyethoxy)phenyl]-6-methoxy-thieno[3,2-c]pyridin-4-yl]-3,4-dihydro-1H-isoquinoline-2-carboxylate: A mixture of tert-butyl 6-(7-bromo-6-methoxy-thieno[3,2-c]pyridin-4-yl)-3,4-dihydro-1H-isoquinoline-2-carboxylate (238 mg, 0.501 mmol), [4-fluoro-2-(2-methoxyethoxy)phenyl]boronic acid (214 mg, 1.00 mmol), tetrakis(triphenylphosphine)palladium(0) (58 mg, 0.050 mmol) and sodium carbonate (106 mg, 1.00 mmol) in 1,4-dioxane (10 mL)/water (1 mL) was stirred at 100° C. under N₂ for 16 hours. The mixture was concentrated and purified by silica gel column (petroleum ether:EA=3:1) to give tert-butyl 6-[7-[4-fluoro-2-(2-methoxyethoxy)phenyl]-6-methoxy-thieno[3,2-c]pyridin-4-yl]-3,4-dihydro-1H-isoquinoline-2-carboxylate (290 mg, 100% yield). LCMS ESI (+) m/z 565.2 (M+H).

Step J: Preparation of 7-[4-fluoro-2-(2-methoxyethoxy)phenyl]-4-(1,2,3,4-tetrahydroisoquinolin-6-yl)thieno[3,2-c]pyridin-6-ol: To a solution of tert-butyl 6-[7-[4-fluoro-2-(2-methoxyethoxy)phenyl]-6-methoxy-thieno[3,2-c]pyridin-4-yl]-3,4-dihydro-1H-isoquinoline-2-carboxylate (2.35 g, 4.16 mmol) in acetonitrile (50 mL) was added sodium iodide (1.87 g, 12.5 mmol) and trimethylchlorosilane (5.30 mL, 41.6 mmol). The mixture was stirred at 80° C. for 1 hour. The mixture was allowed to cool to ambient temperature and Na₂S₂O₃ solution was added. The product was extracted with EtOAc and washed with brine solution. The organic layers were dried over Na₂SO₄ and concentrated to dryness to give crude 7-[4-fluoro-2-(2-methoxyethoxy)phenyl]-4-(1,2,3,4-tetrahydroisoquinolin-6-yl)thieno[3,2-c]pyridin-6-ol (1.70 g, 91% yield) which was used in the next step directly. LCMS ESI (+) m/z 451.1 (M+H).

Step K: Preparation of tert-butyl 6-[7-[4-fluoro-2-(2-methoxyethoxy)phenyl]-6-hydroxy-thieno[3,2-c]pyridin-4-yl]-3,4-dihydro-1H-isoquinoline-2-carboxylate: To a solution of 7-[4-fluoro-2-(2-methoxyethoxy)phenyl]-4-(1,2,3,4-tetrahydroisoquinolin-6-yl)thieno[3,2-c]pyridin-6-ol (1.80 g, 4.00 mmol) and potassium carbonate (1.10 g, 7.99 mmol) in THF (10 mL)/water (10 mL) was added di-tert-butyl dicarbonate (1.10 mL, 4.79 mmol). The mixture was stirred at 25° C. for 3 hours. The mixture was poured into 0.5 N HCl aqueous solution and extracted with EtOAc. The organics were washed with brine, dried over Na₂SO₄, filtered and concentrated. The residue was purified by silica gel column (DCM:MeOH=40:1 to 20:1) to give tert-butyl 6-[7-[4-fluoro-2-(2-methoxyethoxy)phenyl]-6-hydroxy-thieno[3,2-c]pyridin-4-yl]-3,4-dihydro-1H-isoquinoline-2-carboxylate (1.75 g, 80% yield). LCMS ESI (+) m/z 551.2 (M+H).

Step L: Preparation of tert-butyl 6-[7-[4-fluoro-2-(2-methoxyethoxy)phenyl]-6-(trifluoromethylsulfonyloxy)thieno[3,2-c]pyridin-4-yl]-3,4-dihydro-1H-isoquinoline-2-carboxylate: To a solution of tert-butyl 6-[7-[4-fluoro-2-(2-methoxyethoxy)phenyl]-6-hydroxy-thieno[3,2-c]pyridin-4-yl]-3,4-dihydro-1H-isoquinoline-2-carboxylate (170 mg, 0.309 mmol) and N,N-diisopropylethylamine (0.16 mL, 0.93 mmol) in DCM (3 mL) was added trifluoromethanesulfonic anhydride (0.063 mL, 0.37 mmol) at 0° C. The mixture was stirred at 25° C. for 1 hour. The mixture was poured into water and extracted with EtOAc. The organic layers were washed with brine, dried over Na₂SO₄, filtered and concentrated. The residue was purified by silica gel column (petroleum ether:EA=3:1) to give tert-butyl 6-[7-[4-fluoro-2-(2-methoxyethoxy)phenyl]-6-(trifluoromethylsulfonyloxy)thieno[3,2-c]pyridin-4-yl]-3,4-dihydro-1H-isoquinoline-2-carboxylate (130 mg, 62% yield). LCMS ESI (+) m/z 683.1.1 (M+H).

Step M: Preparation of tert-butyl 6-[7-[4-fluoro-2-(2-methoxyethoxy)phenyl]-6-(6-prop-2-enoyl-7,8-dihydro-5H-1,6-naphthyridin-3-yl)thieno[3,2-c]pyridin-4-yl]-3,4-dihydro-1H-isoquinoline-2-carboxylate: To a solution of (6-prop-2-enoyl-7,8-dihydro-5H-1,6-naphthyridin-3-yl)boronic acid (40 mg, 0.10 mmol), tert-butyl 6-[7-[4-fluoro-2-(2-methoxyethoxy)phenyl]-6-(trifluoromethylsulfonyloxy)thieno[3,2-c]pyridin-4-yl]-3,4-dihydro-1H-isoquinoline-2-carboxylate (25 mg, 0.037 mmol) and potassium carbonate (12 mg, 0.087 mmol) in acetonitrile (2 mL) and water (0.2 mL) were added palladium(II) acetate (2.0 mg, 0.0089 mmol) and dicyclohexyl-[2-(2,4,6-triisopropylphenyl)phenyl]phosphane (8.0 mg, 0.017 mmol). The mixture was stirred at 100° C. for 4 h under Ar. The mixture was poured into water and the product was extracted with EtOAc, washed with brine, dried over anhydrous Na₂SO₄ and concentrated. The residue was purified by preparative TLC (EtOAc/petroleum ether: 2/1) to give tert-butyl 6-[7-[4-fluoro-2-(2-methoxyethoxy)phenyl]-6-(6-prop-2-enoyl-7,8-dihydro-5H-1,6-naphthyridin-3-yl)thieno[3,2-c]pyridin-4-yl]-3,4-dihydro-1H-isoquinoline-2-carboxylate (16 mg, 55% yield). LCMS ESI (+) m/z 721.2 (M+H).

Step N: Preparation of 1-[3-[7-[4-fluoro-2-(2-methoxvethoxy)phenyl]-4-(1,2,3,4-tetrahydroisoquinolin-6-yl)thieno[3,2-c]pyridin-6-yl]-7,8-dihydro-5H-1,6-naphthyridin-6-yl]prop-2-en-1-one: To a solution of tert-butyl 6-[7-[4-fluoro-2-(2-methoxyethoxy)phenyl]-6-(6-prop-2-enoyl-7,8-dihydro-5H-1,6-naphthyridin-3-yl)thieno[3,2-c]pyridin-4-yl]-3,4-dihydro-1H-isoquinoline-2-carboxylate (17 mg, 0.021 mmol) in DCM (2 mL) was added TFA (0.5 mL). The mixture was stirred at 25° C. for 1 h. The mixture was concentrated and purified by reverse phase preparative-HPLC to give 1-[3-[7-[4-fluoro-2-(2-methoxyethoxy)phenyl]-4-(1,2,3,4-tetrahydroisoquinolin-6-yl)thieno[3,2-c]pyridin-6-yl]-7,8-dihydro-5H-1,6-naphthyridin-6-yl]prop-2-en-1-one (8.0 mg, 60% yield) as a bis trifluoroacetic acid salt. LCMS ESI (+) m/z 621.2 (M+H). ¹H NMR (400 MHz, CD₃OD) δ 8.49 (s, 1H), 8.12 (s, 1H), 7.85-7.88 (m, 3H), 7.71 (d, J=5.6 Hz, 1H), 7.47 (d, J=8.0 Hz, 1H), 7.42 (dd, J=8.0, 6.6 Hz, 1H), 6.70-6.96 (m, 3H), 6.20-6.32 (m, 1H), 5.81 (d, J=10.8 Hz, 1H), 4.50 (s, 2H), 3.75-4.50 (m, 4H), 3.60 (t, J=6.2 Hz, 2H), 3.40-3.46 (m, 2H), 3.15 (s, 3H), 3.05-3.14 (m, 2H).

### Synthetic Example 11: Synthesis of 1-[3-[4-[7-(4-fluoro-2-methoxy-phenyl)-4-(1,2,3,4-tetrahydroisoquinolin-6-yl)thieno[3,2-c]pyridin-6-yl]pyrazol-1-yl]azetidin-1-yl]prop-2-en-1-one (Compound 82)

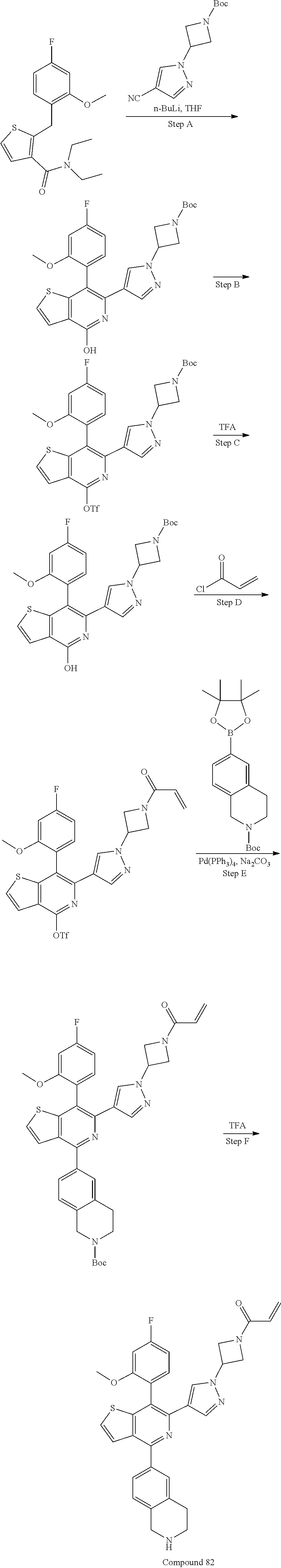

Step A: Preparation of tert-butyl 6-[7-(4-fluoro-2-methoxy-phenyl)-6-[1-(1-prop-2-enoylazetidin-3-yl)pyrazol-4-yl]thieno[3,2-c]pyridin-4-yl]-3,4-dihydro-1H-isoquinoline-2-carboxylate: The title compound was prepared as described in Synthetic Example 7, substituting N,N-diethyl-2-[(4-fluoro-2-methoxy-phenyl)methyl]thiophene-3-carboxamide for N,N-diethyl-2-[[4-fluoro-2-(2-methoxyethoxy)phenyl]methyl]thiophene-3-carboxamide, and substituting tert-butyl 3-(4-cyanopyrazol-1-yl)azetidine-1-carboxylate for 1-(2-trimethylsilylethoxymethyl)pyrazole-4-carbonitrile in Step C. LCMS ESI (+) m/z 558.2 (M+H).

Step B: Preparation of tert-butyl 3-(4-(7-(4-fluoro-2-methoxyphenyl)-4-(((trifluoromethyl)sulfonyl)oxy)thieno[3,2-c]pyridin-6-yl)-1H-pyrazol-1-yl)azetidine-1-carboxylate: To a solution of tert-butyl 3-[4-[7-(4-fluoro-2-methoxy-phenyl)-4-hydroxy-thieno[3,2-c]pyridin-6-yl]pyrazol-1-yl]azetidine-1-carboxylate (176 mg, 0.354 mmol) and pyridine (80 mg, 1.0 mmol) in DCM was added Tf₂O (140 mg, 0.50 mmol). The mixture was stirred at 20° C. for 15 min. The mixture was concentrated and diluted with EtOAc, and washed with NaHCO₃ aqueous solution and brine solution. The organic layers were dried over Na₂SO₄, filtered and concentrated. The residue was purified by preparative TLC (EtOAc/petroleum ether: 1/3) to give tert-butyl 3-(4-(7-(4-fluoro-2-methoxyphenyl)-4-(((trifluoromethyl)sulfonyl)oxy)thieno[3,2-c]pyridin-6-yl)-1H-pyrazol-1-yl)azetidine-1-carboxylate (102 mg, 46% yield).

Step C: Preparation of [7-[4-fluoro-2-(2-methoxyethoxy)phenyl]-6-(4,5,6,7-tetrahydrothiazolo[5,4-c]pyridin-2-yl)thieno[3,2-c]pyridin-4-yl]trifluoromethanesulfonate: To a solution of tert-butyl 3-[4-[7-(4-fluoro-2-methoxy-phenyl)-4-(trifluoromethylsulfonyloxy)thieno[3,2-c]pyridin-6-yl]pyrazol-1-yl]azetidine-1-carboxylate (20 mg, 0.032 mmol) in dichloromethane (3 mL) was added trifluoroacetic acid (1 mL). The mixture was stirred at ambient temperature for 1 h. The solvent was concentrated under reduced pressure to afford [6-[1-(azetidin-3-yl)pyrazol-4-yl]-7-(4-fluoro-2-methoxy-phenyl)thieno[3,2-c]pyridin-4-yl]trifluoromethanesulfonate (27 mg), which was used in the next step without further purification. LCMS ESI (+) m/z 529.0 (M+H).

Step D: Preparation of [7-(4-fluoro-2-methoxy-phenyl)-6-[1-(1-prop-2-enoylazetidin-3-yl)pyrazol-4-yl]thieno[3,2-c]pyridin-4-yl]trifluoromethanesulfonate: To a solution of [6-[1-(azetidin-3-yl)pyrazol-4-yl]-7-(4-fluoro-2-methoxy-phenyl)thieno[3,2-c]pyridin-4-yl]trifluoromethanesulfonate (27 mg, 0.031 mmol) and N,N-diisopropylethylamine (0.016 mL, 0.092 mmol) in DCM (2 mL) was added acryloyl chloride (0.0037 mL, 0.046 mmol). The mixture was stirred at −20° C. for 1 h. The mixture was poured into NaHCO₃ aqueous solution and the product was extracted with EtOAc. The organics were washed with brine, dried over anhydrous Na₂SO₄, concentrated and purified by preparative TLC (EtOAc/petroleum ether: 3/1) to give [7-(4-fluoro-2-methoxy-phenyl)-6-[1-(1-prop-2-enoylazetidin-3-yl)pyrazol-4-yl]thieno[3,2-c]pyridin-4-yl]trifluoromethanesulfonate (17 mg, 95% yield). LCMS ESI (+) m/z 583.1 (M+H).

Step E: Preparation of tert-butyl 6-[7-(4-fluoro-2-methoxy-phenyl)-6-[1-(1-prop-2-enoylazetidin-3-yl)pyrazol-4-yl]thieno[3,2-c]pyridin-4-yl]-3,4-dihydro-1H-isoquinoline-2-carboxylate: To a solution of [7-(4-fluoro-2-methoxy-phenyl)-6-[1-(1-prop-2-enoylazetidin-3-yl) pyrazol-4-yl]thieno[3,2-c]pyridin-4-yl]trifluoromethanesulfonate (17 mg, 0.029 mmol), tert-butyl 6-(4,4,5,5-tetramethyl-1,3,2-dioxaborolan-2-yl)-3,4-dihydro-1H-isoquinoline-2-carboxylate (16 mg, 0.044 mmol) and Na₂CO₃ (7.7 mg, 0.073 mmol) in 1,4-dioxane (2 mL) and water (0.2 mL) was added Pd(PPh₃)₄(5.1 mg, 0.0044 mmol). The mixture was stirred at 90° C. for 4 h under Ar. The mixture was poured into water. The product was extracted with EtOAc, dried over anhydrous Na₂SO₄, concentrated and purified by preparative TLC (EtOAc) to give tert-butyl 6-[7-(4-fluoro-2-methoxy-phenyl)-6-[1-(1-prop-2-enoylazetidin-3-yl)pyrazol-4-yl]thieno[3,2-c]pyridin-4-yl]-3,4-dihydro-1H-isoquinoline-2-carboxylate (16 mg, 82% yield). LCMS ESI (+) m/z 666.3 (M+H).

Step F: Preparation of 1-[3-[4-[7-(4-fluoro-2-methoxy-phenyl)-4-(1,2,3,4-tetrahydroisoquinolin-6-yl)thieno[3,2-c]pyridin-6-yl]pyrazol-1-yl]azetidin-1-yl]prop-2-en-1-one: To a stirred solution of tert-butyl 6-[7-(4-fluoro-2-methoxy-phenyl)-6-[1-(1-prop-2-enoylazetidin-3-yl)pyrazol-4-yl]thieno[3,2-c]pyridin-4-yl]-3,4-dihydro-1H-isoquinoline-2-carboxylate (16 mg, 0.024 mmol) in dichloromethane (3 ml) was added trifluoroacetic acid (1 ml). The mixture was stirred at 25° C. for 1 h. The mixture was concentrated under reduced pressure and purified by reverse phase preparative HPLC to afford 1-[3-[4-[7-(4-fluoro-2-methoxy-phenyl)-4-(1,2,3,4-tetrahydroisoquinolin-6-yl)thieno[3,2-c]pyridin-6-yl]pyrazol-1-yl]azetidin-1-yl]prop-2-en-1-one (6.5 mg, 47% yield) as a bis trifluoroacetic acid salt. LCMS ESI (+) m/z 666.3 (M+H). ¹H NMR (400 MHz, CD₃OD) δ 7.84-7.87 (m, 2H), 7.75 (d, J=5.6 Hz, 1H), 7.71 (s, 1H), 7.64 (d, J=5.6 Hz, 1H), 7.43-7.55 (m, 2H), 7.25-7.39 (m, 1H), 7.04 (dd, J=11.1, 2.2 Hz, 1H), 6.87-6.6.94 (m, 1H), 6.24-6.39 (m, 2H), 5.77 (dd, J=9.9, 2.3 Hz, 1H), 5.20-5.25 (m, 1H), 4.73 (t, J=8.7 Hz, 1H), 4.43-4.57 (m, 4H), 4.27-4.31 (m, 1H), 3.67 (s, 3H), 3.61 (t, J=6.4 Hz, 2H), 3.25-3.37 (m, 2H).

### Synthetic Example 12: Synthesis of 7-[7-(2-methoxy-3-pyridyl)-6-[1-(1-prop-2-enoylazetidin-3-yl)pyrazol-4-yl]thieno[3,2-c]pyridin-4-yl]-2,4-dihydro-1H-isoquinolin-3-one (Compound 84)

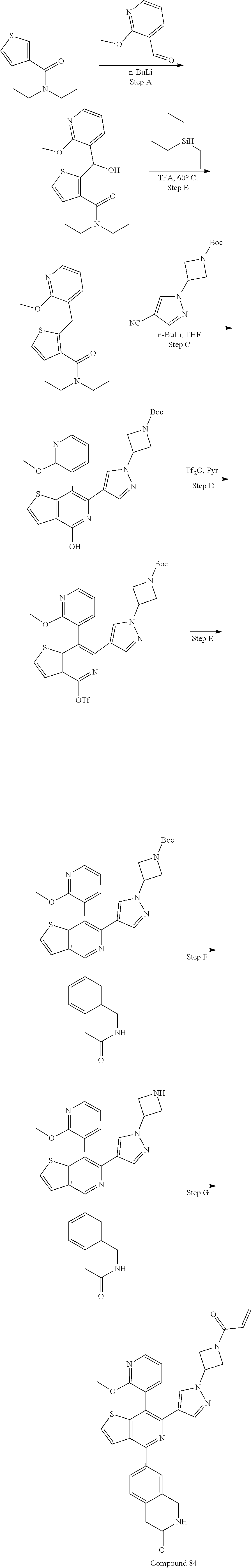

Step A: Preparation of N,N-diethyl-2-[hydroxy-(2-methoxy-3-pyridyl)methyl]thiophene-3-carboxamide: To a solution of N,N-diethylthiophene-3-carboxamide (2.00 g, 10.9 mmol) in THF (10 mL) was added n-butyllithium (5.20 mL, 2.5M in hexanes, 13.1 mmol) slowly at −70° C. The solution was stirred for 1 h at −70° C., and then 2-methoxypyridine-3-carbaldehyde (1.65 g, 12.0 mmol) in THF (4 mL) was added slowly. The solution was stirred for 1 h at rt. The solution was quenched with water and extracted with EtOAc twice. The combined organic layers were washed with brine, dried over Na₂SO₄, and concentrated. The residue was purified by flash column chromatography eluting with 10% EtOAc in hexanes to give N,N-diethyl-2-[hydroxy-(2-methoxy-3-pyridyl)methyl]thiophene-3-carboxamide (2.80 g, 76% yield) as a light yellow oil. LCMS ESI (+) m/z 303.1 (M-17).

Step B: Preparation of N,N-diethyl-2-[(2-methoxy-3-pyridyl)methyl]thiophene-3-carboxamide: To a solution of N,N-diethyl-2-[hydroxy-(2-methoxy-3-pyridyl)methyl]thiophene-3-carboxamide (2.80 g, 8.74 mmol) in TFA (20 mL) was added triethylsilane (11.0 mL, 69.9 mmol). The solution was stirred for 12 h at 60° C. The solution was concentrated. Water was added and the mixture was basified with aqueous NaHCO₃ solution. The mixture was extracted with EtOAc three times. The organic layers were dried over Na₂SO₄, filtered and concentrated. The residue was purified by column chromatography eluting with 10-20% EtOAc in hexanes to give N,N-diethyl-2-[(2-methoxy-3-pyridyl)methyl]thiophene-3-carboxamide (1.90 g, 71% yield) as a light yellow oil. LCMS ESI (+) m/z 305.1 (M+H).

Step C: Preparation of tert-butyl 3-[4-[4-hydroxy-7-(2-methoxy-3-pyridyl)thieno[3,2-c]pyridin-6-yl]pyrazol-1-yl]azetidine-1-carboxylate: The title compound was prepared as described in Synthetic Example 7, substituting N,N-diethyl-2-[(2-methoxy-3-pyridyl)methyl]thiophene-3-carboxamide for N,N-diethyl-2-[[4-fluoro-2-(2-methoxvethoxy)phenyl]methyl]thiophene-3-carboxamide, and substituting tert-butyl 3-(4-cyanopyrazol-1-yl)azetidine-1-carboxylate for 1-(2-trimethylsilylethoxymethyl)pyrazole-4-carbonitrile in Step C. LCMS ESI (+) m/z 480.2 (M+H).

Step D: Preparation of tert-butyl 3-[4-[7-(2-methoxy-3-pyridyl)-4-(trifluoromethylsulfonyloxy)thieno[3,2-c]pyridin-6-yl]pyrazol-1-yl]azetidine-1-carboxylate: To a solution of tert-butyl 3-[4-[4-hydroxy-7-(2-methoxy-3-pyridyl)thieno[3,2-c]pyridin-6-yl]pyrazol-1-yl]azetidine-1-carboxylate (110 mg, 0.161 mmol) and pyridine (0.065 mL, 0.80 mmol) in DCM (2 mL) was added Tf₂O (136 mg, 0.482 mmol). The solution was stirred for 1 h at 30° C. The mixture was poured into water and the product was extracted with EtOAc. The organic layers were dried over Na₂SO₄, filtered and concentrated. The residue was purified by preparative TLC (EtOAc/petroleum ether: 1/1) to give tert-butyl 3-[4-[7-(2-methoxy-3-pyridyl)-4-(trifluoromethylsulfonyloxy)thieno[3,2-c]pyridin-6-yl]pyrazol-1-yl]azetidine-1-carboxylate (90 mg, 87% yield). LCMS ESI (+) m/z 612.1 (M+H).

Step E: Preparation of tert-butyl 3-[4-[7-(2-methoxy-3-pyridyl)-4-(3-oxo-2,4-dihydro-1H-isoquinolin-7-yl)thieno[3,2-c]pyridin-6-yl]pyrazol-1-yl]azetidine-1-carboxylate: To a solution of tert-butyl 3-[4-[7-(2-methoxy-3-pyridyl)-4-(trifluoromethylsulfonyloxy)thieno[3,2-c]pyridin-6-yl]pyrazol-1-yl]azetidine-1-carboxylate (90 mg, 0.14 mmol) and 7-(4,4,5,5-tetramethyl-1,3,2-dioxaborolan-2-yl)-2,4-dihydro-1H-isoquinolin-3-one (76 mg, 0.28 mmol) in 1,4-dioxane (3 mL) and water (0.3 mL) were added Na₂CO₃ (44 mg, 0.42 mmol) and tetrakis(triphenylphosphine)palladium (16 mg, 0.014 mmol). The mixture was stirred for 2 h at 100° C. under N₂. The mixture was filtered through a Celite® pad, and the filtrate was concentrated. The residue was purified by preparative TLC (EtOAc/MeOH: 20/1) to give tert-butyl 3-[4-[7-(2-methoxy-3-pyridyl)-4-(3-oxo-2,4-dihydro-1H-isoquinolin-7-yl)thieno[3,2-c]pyridin-6-yl]pyrazol-1-yl]azetidine-1-carboxylate (80 mg, 89% yield) as a white solid. LCMS ESI (+) m/z 609.3 (M+H).

Step F: Preparation of 7-(6-(1-(azetidin-3-yl)-1H-pyrazol-4-yl)-7-(2-methoxypyridin-3-yl)thieno[3,2-c]pyridin-4-yl)-1,4-dihydroisoquinolin-3(2H)-one: To a solution of tert-butyl 3-[4-[7-(2-methoxy-3-pyridyl)-4-(3-oxo-2,4-dihydro-1H-isoquinolin-7-yl)thieno[3,2-c]pyridin-6-yl]pyrazol-1-yl]azetidine-1-carboxylate (80 mg, 0.13 mmol) in DCM (2 mL) was added TFA (1 mL). The solution was stirred for 1 h at 30° C. The mixture was concentrated to give crude 7-(6-(1-(azetidin-3-yl)-1H-pyrazol-4-yl)-7-(2-methoxypyridin-3-yl)thieno[3,2-c]pyridin-4-yl)-1,4-dihydroisoquinolin-3(2H)-one (100 mg) which was used for next step directly. LCMS ESI (+) m/z 509.2 (M+H).

Step F: Preparation of 7-[7-(2-methoxy-3-pyridyl)-6-[1-(1-prop-2-enoylazetidin-3-yl)pyrazol-4-yl]thieno[3,2-c]pyridin-4-yl]-2,4-dihydro-1H-isoquinolin-3-one: To a solution of 7-[6-[1-(azetidin-3-yl)pyrazol-4-yl]-7-(2-methoxy-3-pyridyl)thieno[3,2-c]pyridin-4-yl]-2,4-dihydro-1H-isoquinolin-3-one (80 mg, 0.13 mmol) in DCM (3 mL) was added TEA (0.056 mL, 0.40 mmol) and prop-2-enoyl prop-2-enoate (25 mg, 0.20 mmol). The mixture was stirred at 30° C. for 1 h. The mixture was concentrated and the residue was purified by preparative TLC (DCM/MeOH: 10/1) to give crude product (20 mg), which was further purified by reverse phase preparative HPLC to give 7-[7-(2-methoxy-3-pyridyl)-6-[1-(1-prop-2-enoylazetidin-3-yl)pyrazol-4-yl]thieno[3,2-c]pyridin-4-yl]-2,4-dihydro-1H-isoquinolin-3-one (4.9 mg, 6% yield) as a bis trifluoroacetic acid salt. LCMS ESI (+) m/z 563.3 (M+H). ¹H NMR (400 MHz, CD₃OD) δ 8.37-8.39 (d, J=6.8 Hz, 1H), 7.87-7.88 (m, 4H), 7.80-7.82 (d, J=9.2 Hz, 1H), 7.73-7.74 (d, J=5.6 Hz, 1H), 7.47-7.53 (m, 2H), 7.17-7.20 (m, 1H), 6.25-6.39 (m, 2H), 5.75-5.78 (m, 1H), 5.23-5.28 (m, 1H), 4.71-4.75 (m, 1H), 4.64 (s, 2H), 4.57-4.58 (m, 2H), 4.46-4.51 (m, 2H), 4.29-4.32 (m, 1H), 3.78 (s, 1H), 3.73 (s, 1H).

### Synthetic Example 13: Synthesis of 1-[2-[7-(4-fluoro-2-methoxy-phenyl)-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (Compound 87)

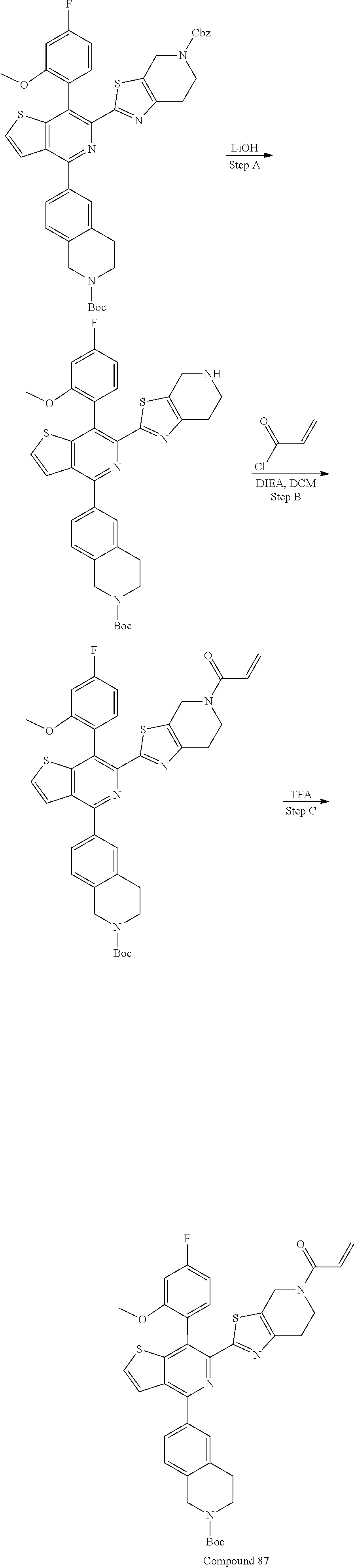

Step A: Preparation of tert-butyl 6-(7-(4-fluoro-2-methoxyphenyl)-6-(4,5,6,7-tetrahydrothiazolo[5,4-c]pyridin-2-yl)thieno[3,2-c]pyridin-4-yl)-3,4-dihydroisoquinoline-2(1H)-carboxylate: To a solution of benzyl 2-[4-(2-tert-butoxycarbonyl-3,4-dihydro-1H-isoquinolin-6-yl)-7-(4-fluoro-2-methoxy-phenyl)thieno[3,2-c]pyridin-6-yl]-6,7-dihydro-4H-thiazolo[5,4-c]pyridine-5-carboxylate (prepared as described in Synthetic Example 9 Step D to L, substituting 4-fluoro-N,2-dimethoxy-N-methyl-benzamide for 4-fluoro-N-methoxy-2-(2-methoxyethoxy)-N-methyl-benzamide in Step D, 31 mg, 0.041 mmol) in 1,4-dioxane (1 mL) and water (1 mL) was added lithium hydroxide monohydrate (100 mg, 2.50 mmol). The mixture was stirred at 95° C. for 16 hours. The mixture was poured into water and the product was extracted with EtOAc. The organic layers were washed with brine, dried over anhydrous Na₂SO₄, concentrated and purified by preparative TLC (EtOAc/petroleum ether: 1/6) to give tert-butyl 6-(7-(4-fluoro-2-methoxyphenyl)-6-(4,5,6,7-tetrahydrothiazolo[5,4-c]pyridin-2-yl)thieno[3,2-c]pyridin-4-yl)-3,4-dihydroisoquinoline-2(1H)-carboxylate (27 mg, crude). LCMS ESI (+) m/z 629.2 (M+H).

Step B: Preparation of tert-butyl 6-[7-(4-fluoro-2-methoxy-phenyl)-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate: To a solution of tert-butyl 6-[7-(4-fluoro-2-methoxy-phenyl)-6-(4,5,6,7-tetrahydrothiazolo[5,4-c]pyridin-2-yl)thieno[3,2-c]pyridin-4-yl]-3,4-dihydro-1H-isoquinoline-2-carboxylate (20 mg, 0.032 mmol) in DCM (5 mL) were added triethylamine (0.022 mL, 0.16 mmol) and acryloyl chloride (0.0031 mL, 0.038 mmol). The mixture was stirred at 25° C. for 0.5 h. The mixture was poured into NaHCO₃ aqueous solution (30 mL) and the product was extracted with EtOAc. The combined organic layers were washed with brine, dried over Na₂SO₄ and concentrated. The residue was purified by preparative TLC (petroleum ether:EtOAc=1:1) to give tert-butyl 6-[7-(4-fluoro-2-methoxy-phenyl)-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (14 mg, 64% yield). LCMS ESI (+) m/z 683 (M+H).

Step C: Preparation of 1-[2-[7-(4-fluoro-2-methoxy-phenyl)-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one: To a solution of tert-butyl 6-[7-(4-fluoro-2-methoxy-phenyl)-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (14 mg, 0.021 mmol) in DCM (4 mL) was added trifluoroacetic acid (1 mL). The mixture was stirred at 25° C. for 1 h. The mixture was concentrated, and the residue was purified by reverse phase preparative HPLC to give 1-[2-[7-(4-fluoro-2-methoxy-phenyl)-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (5.0 mg, 42% yield) as a bis trifluoroacetic acid salt. LCMS ESI (+) m/z 583.2 (M+H). ¹H NMR (400 MHz, CD₃OD) δ 7.90-7.91 (m, 2H), 7.84 (d, J=5.6 Hz, 1H), 7.73 (d, J=5.6 Hz, 1H), 7.48 (d, J=7.6 Hz, 1H), 7.34 (dd, J==8.4, 6.8 Hz, 1H), 6.84-6.95 (m, 3H), 6.24-6.28 (m, 1H), 5.79-5.83 (m, 1H), 4.52 (s, 2H), 3.93 (t, J=5.2 Hz, 2H), 3.58-3.66 (m, 6H), 2.70-2.78 (m, 2H).

### Synthetic Example 14: Synthesis of 1-[3-[4-(4-fluoro-2-methoxy-phenyl)-1-(1,2,3,4-tetrahydroisoquinolin-6-yl)-6,7-dihydro-5H-cyclopenta[c]pyridin-3-yl]-7,8-dihydro-5H-1,6-naphthyridin-6-yl]prop-2-en-1-one (Compound 89)

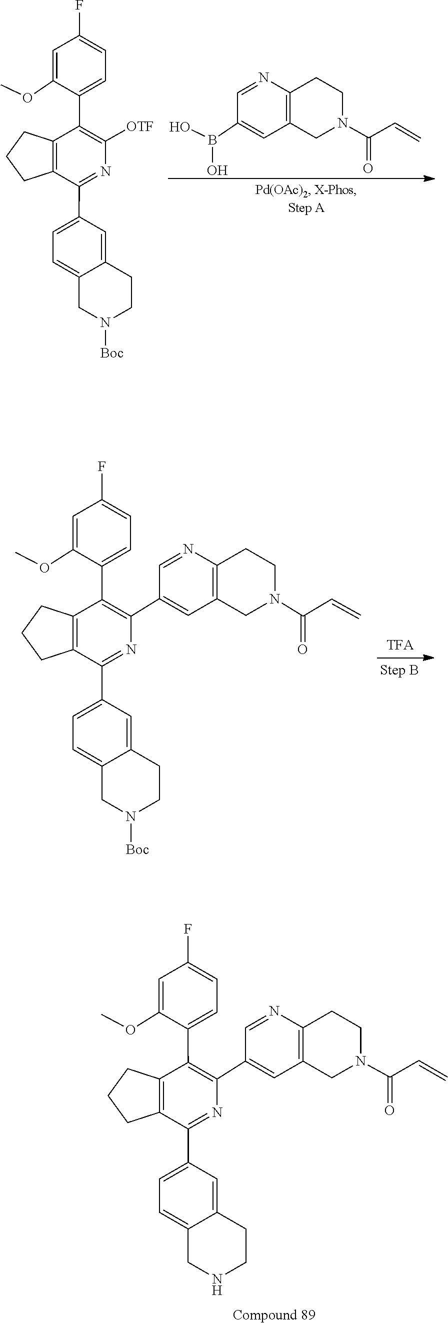

Step A: Preparation of tert-butyl 6-[4-(4-fluoro-2-methoxy-phenyl)-3-(6-prop-2-enoyl-7,8-dihydro-5H-1,6-naphthyridin-3-yl)-6,7-dihydro-5H-cyclopenta[c]pyridin-1-yl]-3,4-dihydro-1H-isoquinoline-2-carboxylate: To a solution of (6-prop-2-enoyl-7,8-dihydro-5H-1,6-naphthyridin-3-yl)boronic acid (50 mg, 0.086 mmol), tert-butyl 6-[4-(4-fluoro-2-methoxy-phenyl)-3-(trifluoromethylsulfonyloxy)-6,7-dihydro-5H-cyclopenta[c]pyridin-1-yl]-3,4-dihydro-JH-isoquinoline-2-carboxylate (prepared as described in Synthetic Example 1 Steps C to I, substituting (4-fluoro-2-methoxy-phenyl)boronic acid for [2-(2-methoxyethoxy)phenyl]boronic acid in Step C, 25 mg, 0.040 mmol) and potassium carbonate (12 mg, 0.087 mmol) in acetonitrile (2 mL) and water (0.2 mL) were added palladium(II) acetate (1.2 mg, 0.0053 mmol) and dicyclohexyl-[2-(2,4,6-triisopropylphenyl)phenyl]phosphane (5.0 mg, 0.011 mmol). The mixture was stirred at 100° C. for 2 hours under Ar. The mixture was poured into water. The product was extracted with EtOAc, dried over anhydrous Na₂SO₄, concentrated and purified by preparative TLC (EtOAc:petroleum ether=1:1) to give tert-butyl 6-[4-(4-fluoro-2-methoxy-phenyl)-3-(6-prop-2-enoyl-7,8-dihydro-5H-1,6-naphthyridin-3-yl)-6,7-dihydro-5H-cyclopenta[c]pyridin-1-yl]-3,4-dihydro-1H-isoquinoline-2-carboxylate (11 mg, 41% yield). LCMS ESI (+) m/z 661.3 (M+H).

Step B: Preparation of 1-[3-[4-(4-fluoro-2-methoxy-phenyl)-1-(1,2,3,4-tetrahydroisoquinolin-6-yl)-6,7-dihydro-5H-cyclopenta[c]pyridin-3-yl]-7,8-dihydro-5H-1,6-naphthyridin-6-yl]prop-2-en-1-one: To a solution of tert-butyl 6-[4-(4-fluoro-2-methoxy-phenyl)-3-(6-prop-2-enoyl-7,8-dihydro-5H-1,6-naphthyridin-3-yl)-6,7-dihydro-5H-cyclopenta[c]pyridin-1-yl]-3,4-dihydro-1H-isoquinoline-2-carboxylate (11 mg, 0.017 mmol) in DCM (2 mL) was added TFA (0.5 mL). The mixture was stirred at 25° C. for 1 h. The mixture was concentrated and purified by reverse phase preparative HPLC to give 1-[3-[4-(4-fluoro-2-methoxy-phenyl)-1-(1,2,3,4-tetrahydroisoquinolin-6-yl)-6,7-dihydro-5H-cyclopenta[c]pyridin-3-yl]-7,8-dihydro-5H-1,6-naphthyridin-6-yl]prop-2-en-1-one (5.7 mg, 0.0101 mmol, 60.39% yield) as a bis trifluoroacetic acid salt. LCMS ESI (+) m/z 561.3 (M+H). ¹H NMR (400 MHz, CD₃OD) δ 8.34 (s, 1H), 7.95-8.01 (m, 1H), 7.75-7.78 (m, 2H) 7.39 (d, J=8.0 Hz, 1H), 7.10 (dd, J=8.4, 6.7 Hz, 1H), 6.70-6.89 (m, 3H), 6.20-6.31 (m, 1H), 5.81 (d, J=10.2 Hz, 1H), 4.46 (s, 2H), 3.98 (t, J=6.0 Hz, 2H), 3.66 (s, 3H), 3.57 (t, J=6.4 Hz, 2H), 3.18-3.27 (m, 4H), 3.00-3.12 (m, 2H), 2.73-2.92 (m, 2H), 2.12-2.16 (m, 2H).

### Synthetic Example 15: Synthesis of 1-[2-[4-(4-fluoro-2-methoxy-phenyl)-1-(1,2,3,4-tetrahydroisoquinolin-6-yl)-6,7-dihydro-5H-cyclopenta[c]pyridin-3-yl]-6,7-dihydro-4H-thiazolo[5,4-c]pyridin-5-yl]prop-2-en-1-one (Compound 96)

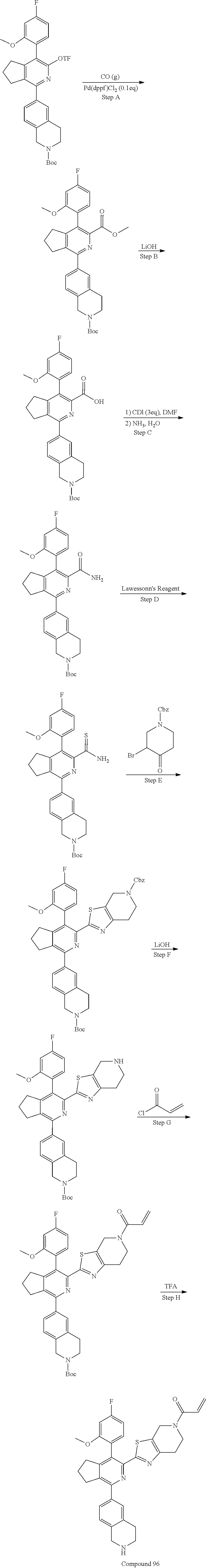

Step A: Preparation of tert-butyl 6-[4-(4-fluoro-2-methoxy-phenyl)-3-methoxycarbonyl-6,7-dihydro-5H-cyclopenta[c]pyridin-1-yl]-3,4-dihydro-1H-isoquinoline-2-carboxylate: A mixture of tert-butyl 6-[4-(4-fluoro-2-methoxy-phenyl)-3-(trifluoromethylsulfonyloxy)-6,7-dihydro-5H-cyclopenta[c]pyridin-1-yl]-3,4-dihydro-1H-isoquinoline-2-carboxylate (300 mg, 0.482 mmol), TEA (0.340 mL, 2.41 mmol) and Pd(dppf)Cl₂ (35 mg, 0.048 mmol) in methanol (20 mL) was stirred at 80° C. under CO atmosphere for 6 hours. The mixture was concentrated and purified by silica gel column (petroleum ether:EtOAc=5:1 to 2:1) to get tert-butyl 6-[4-(4-fluoro-2-methoxy-phenyl)-3-methoxycarbonyl-6,7-dihydro-5H-cyclopenta[c]pyridin-1-yl]-3,4-dihydro-1H-isoquinoline-2-carboxylate (270 mg, 100%). LCMS ESI (+) m/z 533.2 (M+H).

Step B: Preparation of 1-(2-tert-butoxycarbonyl-3,4-dihydro-1H-isoquinolin-6-yl)-4-(4-fluoro-2-methoxy-phenyl)-6,7-dihydro-5H-cyclopenta[c]pyridine-3-carboxylic acid: To a solution of tert-butyl 6-[4-(4-fluoro-2-methoxy-phenyl)-3-methoxycarbonyl-6,7-dihydro-5H-cyclopenta[c]pyridin-1-yl]-3,4-dihydro-1H-isoquinoline-2-carboxylate (20 mg, 0.038 mmol) in THF (1 mL)/methanol (1 mL)/water (0.5 mL) was added lithium hydroxide monohydrate (30 mg, 0.75 mmol). The mixture was stirred at 25° C. for 2 hours. 1 N HCl aqueous solution was added to adjust pH to 3-5, and the product was extracted with EtOAc. The organic layers were washed with brine, dried over Na₂SO₄, filtered and concentrated to dryness to give crude 1-(2-tert-butoxycarbonyl-3,4-dihydro-1H-isoquinolin-6-yl)-4-(4-fluoro-2-methoxy-phenyl)-6,7-dihydro-5H-cyclopenta[c]pyridine-3-carboxylic acid (20 mg, 103% yield), which was used in the next step directly. LCMS ESI (+) m/z 519.2 (M+H).

Step C: Preparation of tert-butyl 6-[3-carbamoyl-4-(4-fluoro-2-methoxy-phenyl)-6,7-dihydro-5H-cyclopenta[c]pyridin-1-yl]-3,4-dihydro-1H-isoquinoline-2-carboxylate: To a solution 1-(2-tert-butoxycarbonyl-3,4-dihydro-1H-isoquinolin-6-yl)-4-(4-fluoro-2-methoxy-phenyl)-6,7-dihydro-5H-cyclopenta[c]pyridine-3-carboxylic acid (340 mg, 0.656 mmol) in DMF (2 mL) was added CDI (319 mg, 1.97 mmol). The mixture was stirred at 25° C. for 1 hour. The mixture was added to ammonia aqueous solution, and the resulting mixture was stirred for additional 1 h. The mixture was diluted with EtOAc and washed by water and brine. The organic layers were dried over Na₂SO₄, filtered and concentrated to give tert-butyl 6-[3-carbamoyl-4-(4-fluoro-2-methoxy-phenyl)-6,7-dihydro-5H-cyclopenta[c]pyridin-1-yl]-3,4-dihydro-TH-isoquinoline-2-carboxylate (277 mg, 82% yield). LCMS ESI (+) m/z 518.2 (M+H).

Step D: Preparation of tert-butyl 6-[3-carbamothioyl-4-(4-fluoro-2-methoxy-phenyl)-6,7-dihydro-5H-cyclopenta[c]pyridin-1-yl]-3,4-dihydro-1H-isoquinoline-2-carboxylate: A mixture of tert-butyl 6-[3-carbamoyl-4-(4-fluoro-2-methoxy-phenyl)-6,7-dihydro-5H-cyclopenta[c]pyridin-1-yl]-3,4-dihydro-1H-isoquinoline-2-carboxylate (160 mg, 0.309 mmol) and (2,4-bis(4-methoxyphenyl)-1,3-dithia-2,4-diphosphetane-2,4-disulfide) (138 mg, 0.340 mmol) in THF (6 mL) was stirred at 70° C. for 10 hours. The mixture was diluted with EtOAc and then washed with NaHCO₃ aqueous solution and brine solution. The organic layers were dried over Na₂SO₄, filtered, concentrated and purified by silica gel column (petroleum ether:EtOAc=2:1 to 1:1) to give tert-butyl 6-[3-carbamothioyl-4-(4-fluoro-2-methoxy-phenyl)-6,7-dihydro-5H-cyclopenta[c]pyridin-1-yl]-3,4-dihydro-1H-isoquinoline-2-carboxylate (140 mg, 85% yield). LCMS ESI (+) m/z 534.2 (M+H).

Step E: Preparation of benzyl 2-[1-(2-tert-butoxycarbonyl-3,4-dihydro-1H-isoquinolin-6-yl)-4-(4-fluoro-2-methoxy-phenyl)-6,7-dihydro-5H-cyclopenta[c]pyridin-3-yl]-6,7-dihydro-4H-thiazolo[5,4-c]pyridine-5-carboxylate: A mixture of tert-butyl 6-[3-carbamothioyl-4-(4-fluoro-2-methoxy-phenyl)-6,7-dihydro-5H-cyclopenta[c]pyridin-1-yl]-3,4-dihydro-1H-isoquinoline-2-carboxylate (76 mg, 0.14 mmol) and benzyl 3-bromo-4-oxo-piperidine-1-carboxylate (89 mg, 0.29 mmol) in ethanol (4 mL) was stirred at 70° C. under N₂ for 12 hours. The mixture was concentrated and purified by preparative TLC (petroleum ether:EtOAc=2:1) to give benzyl 2-[1-(2-tert-butoxycarbonyl-3,4-dihydro-1H-isoquinolin-6-yl)-4-(4-fluoro-2-methoxy-phenyl)-6,7-dihydro-5H-cyclopenta[c]pyridin-3-yl]-6,7-dihydro-4H-thiazolo[5,4-c]pyridine-5-carboxylate (93 mg, 87% yield). LCMS ESI (+) m/z 747.3 (M+H).

Step F: Preparation of tert-butyl 6-[4-(4-fluoro-2-methoxy-phenyl)-3-(4,5,6,7-tetrahydrothiazolo[5,4-c]pyridin-2-yl)-6,7-dihydro-5H-cyclopenta[c]pyridin-1-yl]-3,4-dihydro-1H-isoquinoline-2-carboxylate: To a solution of benzyl 2-[1-(2-tert-butoxycarbonyl-3,4-dihydro-1H-isoquinolin-6-yl)-4-(4-fluoro-2-methoxy-phenyl)-6,7-dihydro-5H-cyclopenta[c]pyridin-3-yl]-6,7-dihydro-4H-thiazolo[5,4-c]pyridine-5-carboxylate (93 mg, 0.13 mmol) in 1,4-dioxane (1 mL)/water (1 mL) was added lithium hydroxide monohydrate (100 mg, 2.50 mmol). The mixture was stirred at 95° C. for 16 hours. The mixture was poured into water and extracted with EtOAc. The organics were washed with brine, dried over Na₂SO₄, filtered and concentrated to dryness to give tert-butyl 6-[4-(4-fluoro-2-methoxy-phenyl)-3-(4,5,6,7-tetrahydrothiazolo[5,4-c]pyridin-2-yl)-6,7-dihydro-5H-cyclopenta[c]pyridin-1-yl]-3,4-dihydro-1H-isoquinoline-2-carboxylate (61 mg, 80% yield). LCMS ESI (+) m/z 613.3 (M+H).

Step G: Preparation of tert-butyl 6-[4-(4-fluoro-2-methoxy-phenyl)-3-(5-prop-2-enoyl-6,7-dihydro-4H-thiazolo[5,4-c]pyridin-2-yl)-6,7-dihydro-5H-cyclopenta[c]pyridin-1-yl]-3,4-dihydro-1H-isoquinoline-2-carboxylate: To a solution of tert-butyl 6-[4-(4-fluoro-2-methoxy-phenyl)-3-(4,5,6,7-tetrahydrothiazolo[5,4-c]pyridin-2-yl)-6,7-dihydro-5H-cyclopenta[c]pyridin-1-yl]-3,4-dihydro-1H-isoquinoline-2-carboxylate (22 mg, 0.036 mmol) and DIPEA (0.019 mL, 0.11 mmol) in DCM (2 mL) was added acryloyl chloride (0.0035 mL, 0.043 mmol) at 0° C. The mixture was stirred at 25° C. for 1 hour. NaHCO₃ solution was added to quench the reaction, and the product was extracted with DCM. The organic layers were dried over Na₂SO₄, filtered and concentrated. The residue was purified by preparative TLC (petroleum ether:EtOAc=1:1) to give tert-butyl 6-[4-(4-fluoro-2-methoxy-phenyl)-3-(5-prop-2-enoyl-6,7-dihydro-4H-thiazolo[5,4-c]pyridin-2-yl)-6,7-dihydro-5H-cyclopenta[c]pyridin-1-yl]-3,4-dihydro-1H-isoquinoline-2-carboxylate (16 mg, 67% yield). LCMS ESI (+) m/z 667.3 (M+H).

Step H: Preparation of 1-[2-[4-(4-fluoro-2-methoxy-phenyl)-1-(1,2,3,4-tetrahydroisoquinolin-6-yl)-6,7-dihydro-5H-cyclopenta[c]pyridin-3-yl]-6,7-dihydro-4H-thiazolo[5,4-c]pyridin-5-yl]prop-2-en-1-one: To a solution of tert-butyl 6-[4-(4-fluoro-2-methoxy-phenyl)-3-(5-prop-2-enoyl-6,7-dihydro-4H-thiazolo[5,4-c]pyridin-2-yl)-6,7-dihydro-5H-cyclopenta[c]pyridin-1-yl]-3,4-dihydro-1H-isoquinoline-2-carboxylate (16 mg, 0.024 mmol) in DCM (2 mL) was added trifluoroacetic acid (0.5 mL). The mixture was stirred at 25° C. for 1 hour. The mixture was concentrated and purified by reverse phase preparative HPLC to give 1-[2-[4-(4-fluoro-2-methoxy-phenyl)-1-(1,2,3,4-tetrahydroisoquinolin-6-yl)-6,7-dihydro-5H-cyclopenta[c]pyridin-3-yl]-6,7-dihydro-4H-thiazolo[5,4-c]pyridin-5-yl]prop-2-en-1-one (12 mg, 91% yield) as a bis trifluoroacetic acid salt. LCMS ESI (+) m/z 563.3 (M+H). ¹H NMR (400 MHz, CD₃OD) δ 8.37-8.39 (d, J=6.8 Hz, 1H), 7.87-7.88 (m, 4H), 7.80-7.82 (d, J=9.2 Hz, 1H), 7.73-7.74 (d, J=5.6 Hz, 1H), 7.47-7.53 (m, 2H), 7.17-7.20 (m, 1H), 6.25-6.39 (m, 2H), 5.75-5.78 (m, 1H), 5.23-5.28 (m, 1H), 4.71-4.75 (m, 1H), 4.64 (s, 2H), 4.57-4.58 (m, 2H), 4.46-4.51 (m, 2H), 4.29-4.32 (m, 1H), 3.78 (s, 1H), 3.73 (s, 1H).

### Synthetic Example 16: Synthesis of 1-[3-[4-[4-(4-fluoro-2-methoxy-phenyl)-1-(1,2,3,4-tetrahydroisoquinolin-6-yl)-6,7-dihydro-5H-cyclopenta[c]pyridin-3-yl]pyrazol-1-yl]azetidin-1-yl]prop-2-en-1-one (Compound 97)

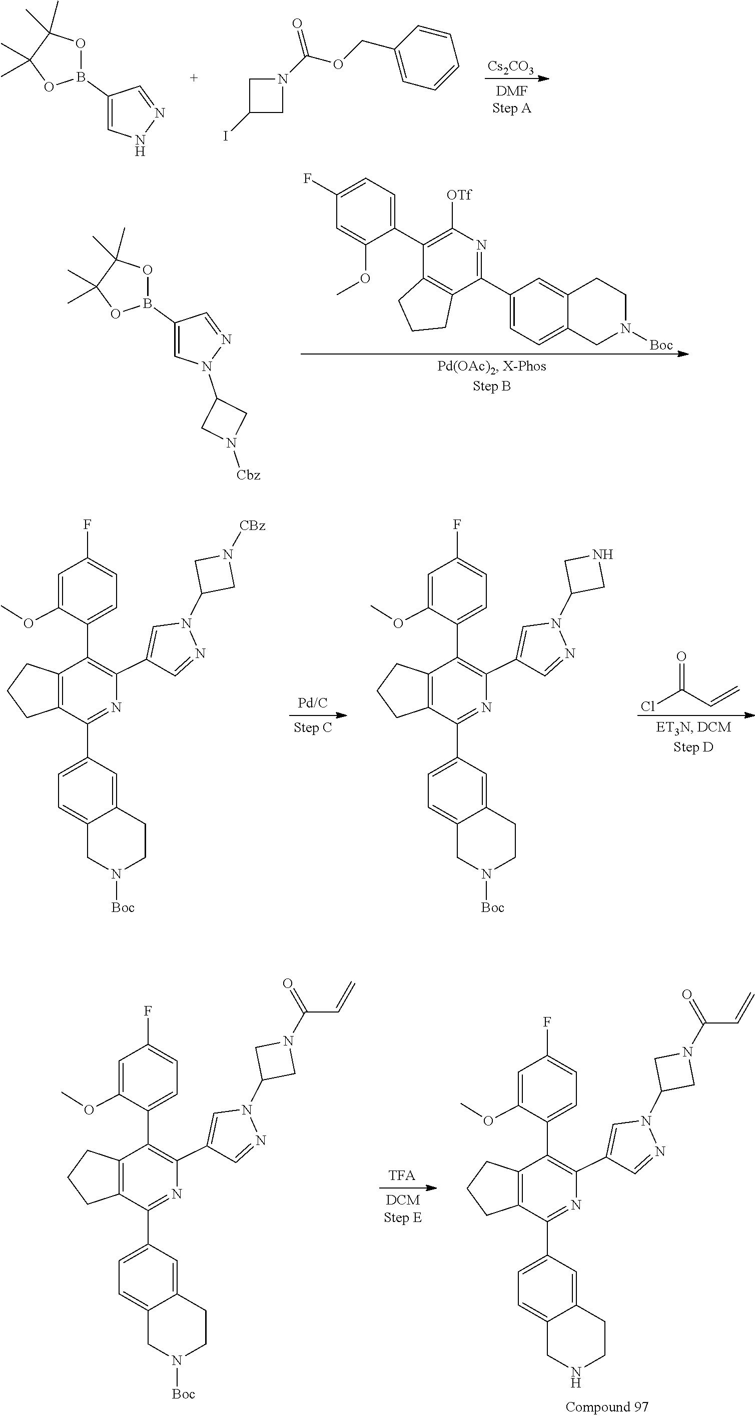

Step A: Preparation of benzyl 3-[4-(4,4,5,5-tetramethyl-1,3,2-dioxaborolan-2-yl) pyrazol-1-yl]azetidine-1-carboxylate: A suspension of 4-(4,4,5,5-tetramethyl-1,3,2-dioxaborolan-2-yl)-1H-pyrazole (200 mg, 1.03 mmol), benzyl 3-iodoazetidine-1-carboxylate (360 mg, 1.13 mmol) and cesium carbonate (672 mg, 2.06 mmol) in DMF (4 mL) was stirred at 95° C. for 5 hours. The mixture was diluted with water. The product was extracted with EtOAc, washed with brine, dried over Na₂SO₄, filtered and concentrated to dryness to afford crude benzyl 3-[4-(4,4,5,5-tetramethyl-1,3,2-dioxaborolan-2-yl)pyrazol-1-yl]azetidine-1-carboxylate (360 mg, 91% yield) which was used in the next step directly.

Step B: Preparation of tert-butyl 6-[3-[1-(1-benzyloxycarbonylazetidin-3-yl)pyrazol-4-yl]-4-(4-fluoro-2-methoxy-phenyl)-6,7-dihydro-5H-cyclopenta[c]pyridin-1-yl]-3,4-dihydro-1H-isoquinoline-2-carboxylate: A mixture of tert-butyl 6-[4-(4-fluoro-2-methoxy-phenyl)-3-(trifluoromethylsulfonyloxy)-6,7-dihydro-5H-cyclopenta[c]pyridin-1-yl]-3,4-dihydro-1H-isoquinoline-2-carboxylate (50 mg, 0.080 mmol), benzyl 3-[4-(4,4,5,5-tetramethyl-1,3,2-dioxaborolan-2-yl)pyrazol-1-yl]azetidine-1-carboxylate (37 mg, 0.096 mmol), palladium(II) acetate (1.8 mg, 0.0080 mmol), X-PHOS (7.7 mg, 0.016 mmol) and K₂CO₃ (3.9 mg, 0.16 mmol) in acetonitrile (4 mL)/water (0.4 mL) was stirred at 100° C. for 4 hours under N₂. The mixture was filtered and concentrated. The residue was purified by preparative TLC (petroleum ether:EtOAc=1:1) to give tert-butyl 6-[3-[1-(1-benzyloxycarbonylazetidin-3-yl)pyrazol-4-yl]-4-(4-fluoro-2-methoxy-phenyl)-6,7-dihydro-5H-cyclopenta[c]pyridin-1-yl]-3,4-dihydro-1H-isoquinoline-2-carboxylate (30 mg, 51% yield). LCMS ESI (+) m/z 730.3 (M+H).

Step C: Preparation of tert-butyl 6-[3-[1-(azetidin-3-yl)pyrazol-4-yl]-4-(4-fluoro-2-methoxy-phenyl)-6,7-dihydro-5H-cyclopenta[c]pyridin-1-yl]-3,4-dihydro-1H-isoquinoline-2-carboxylate: To a solution of tert-butyl 6-[3-[1-(1-benzyloxycarbonylazetidin-3-yl)pyrazol-4-yl]-4-(4-fluoro-2-methoxy-phenyl)-6,7-dihydro-5H-cyclopenta[c]pyridin-1-yl]-3,4-dihydro-1H-isoquinoline-2-carboxylate (30 mg, 0.041 mmol) in methanol (4 mL) was added Pd/C (5.0 mg, 0.041 mmol). The mixture was stirred at 25° C. for 12 hours. The mixture was filtered through Celite® and concentrated to give tert-butyl 6-[3-[1-(azetidin-3-yl)pyrazol-4-yl]-4-(4-fluoro-2-methoxy-phenyl)-6,7-dihydro-5H-cyclopenta[c]pyridin-1-yl]-3,4-dihydro-1H-isoquinoline-2-carboxylate (24 mg, 98% yield). LCMS ESI (+) m/z 596.3 (M+H).

Step D: Preparation of tert-butyl 6-[4-(4-fluoro-2-methoxy-phenyl)-3-[1-(1-prop-2-enoylazetidin-3-yl)pyrazol-4-yl]-6,7-dihydro-5H-cyclopenta[c]pyridin-1-yl]-3,4-dihydro-1H-isoquinoline-2-carboxylate: To a solution of tert-butyl 6-[3-[1-(azetidin-3-yl)pyrazol-4-yl]-4-(4-fluoro-2-methoxy-phenyl)-6,7-dihydro-5H-cyclopenta[c]pyridin-1-yl]-3,4-dihydro-1H-isoquinoline-2-carboxylate (24 mg, 0.040 mmol) and DIPEA (0.021 mL, 0.12 mmol) in DCM (2 mL) was added acryloyl chloride (0.0039 mL, 0.048 mmol). The mixture was stirred at 25° C. for 1 hour. NaHCO₃ solution was added to quench the reaction, and the product was extracted with DCM. The organic phase was concentrated and purified by preparative TLC (petroleum ether:EtOAc=1:2) to give tert-butyl 6-[4-(4-fluoro-2-methoxy-phenyl)-3-[1-(1-prop-2-enoylazetidin-3-yl)pyrazol-4-yl]-6,7-dihydro-5H-cyclopenta[c]pyridin-1-yl]-3,4-dihydro-1H-isoquinoline-2-carboxylate (18 mg, 69% yield). LCMS ESI (+) m/z 650.3 (M+H).

Step E: Preparation of 1-[3-[4-[4-(4-fluoro-2-methoxy-phenyl)-1-(1,2,3,4-tetrahydroisoquinolin-6-yl)-6,7-dihydro-5H-cyclopenta[c]pyridin-3-yl]pyrazol-1-yl]azetidin-1-yl]prop-2-en-1-one: To a solution of tert-butyl 6-[4-(4-fluoro-2-methoxy-phenyl)-3-[1-(1-prop-2-enoylazetidin-3-yl)pyrazol-4-yl]-6,7-dihydro-5H-cyclopenta[c]pyridin-1-yl]-3,4-dihydro-1H-isoquinoline-2-carboxylate (18 mg, 0.028 mmol) in DCM (2 mL) was added trifluoroacetic acid (0.5 mL). The mixture was stirred at 25° C. for 1 hour. The mixture was concentrated and purified by reverse phase preparative HPLC to give 1-[3-[4-[4-(4-fluoro-2-methoxy-phenyl)-1-(1,2,3,4-tetrahydroisoquinolin-6-yl)-6,7-dihydro-5H-cyclopenta[c]pyridin-3-yl]pyrazol-1-yl]azetidin-1-yl]prop-2-en-1-one (5.5 mg, 36% yield) as a bis trifluoroacetic acid salt. LCMS ESI (+) m/z 550.3 (M+H). ¹H NMR (400 MHz, CD₃OD) δ 7.75-7.80 (m, 3H), 7.46-7.51 (m, 2H), 7.16 (d, J=6.4 Hz, 1H), 6.97 (dd, J=10.8, 2.2 Hz, 1H), 6.84 (dt, J=8.3, 2.4 Hz, 1H), 6.24-6.38 (m, 2H), 5.77 (dd, J=9.8, 2.4 Hz, 1H), 5.21-5.23 (m, 1H), 4.70-4.75 (m, 2H), 4.45-4.55 (m, 3H), 4.27 (dd, J=11.0, 5.3 Hz, 1H), 3.70 (s, 3H), 3.58 (t, J=6.4 Hz, 2H), 3.17-3.28 (m, 4H), 2.83-2.91 (m, 2H), 2.13-2.20 (m, 2H).

### Synthetic Example 17: Synthesis of 1-[3-[4-[7-(4-fluoro-2-methoxy-phenyl)-4-(1,2,3,4-tetrahydroisoquinolin-6-yl)thiazolo[4,5-c]pyridin-6-yl]pyrazol-1-yl]azetidin-1-yl]prop-2-en-1-one (Compound 104)

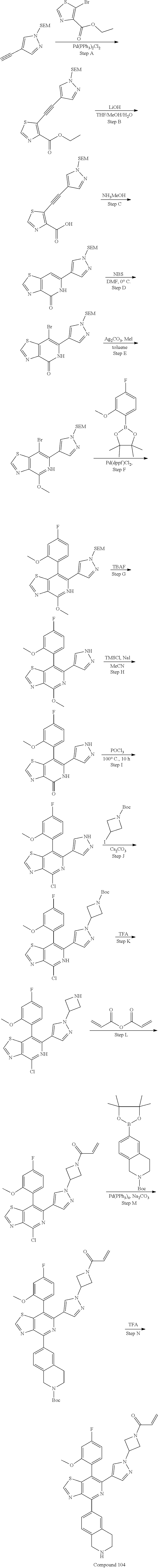

Step A: Preparation of ethyl 5-[2-[1-(2-trimethylsilylethoxymethyl)pyrazol-4-yl]ethynyl]thiazole-4-carboxylate: A solution of 2-[(4-ethynylpyrazol-1-yl)methoxy]ethyl-trimethylsilane (2.40 g, 10.8 mmol), ethyl 5-bromothiazole-4-carboxylate (4.59 g, 19.4 mmol), 1,1′-bis(diphenylphosphino)ferrocenedichloro palladium(II) dichloromethane complex (790 mg, 1.08 mmol), copper(I) iodide (206 mg, 1.08 mmol) and triethylamine (4.50 mL, 32.4 mmol) in THF (6 mL) was stirred for 6 h at 50° C. The mixture was diluted with water (70 mL) and the product was extracted with EtOAc (80 ml×3). The organic layers were washed with 70 mL of saturated brine solution, separated and dried (Na₂SO₄) before concentration to dryness. The residue was then purified by flash column chromatography (petroleum ether/EtOAc=4/1) to give ethyl 5-[2-[1-(2-trimethylsilylethoxymethyl)pyrazol-4-yl]ethynyl]thiazole-4-carboxylate (2.80 g, 69% yield) as a light yellow solid. LCMS ESI (+) m/z 378.1 (M+H).

Step B: Preparation of 5-[2-[1-(2-trimethylsilylethoxymethyl)pyrazol-4-yl]ethynyl]thiazole-4-carboxylic acid: To a solution of ethyl 5-[2-[1-(2-trimethylsilylethoxymethyl)pyrazol-4-yl]ethynyl]thiazole-4-carboxylate (2.80 g, 7.42 mmol) in THF (30 mL), methanol (10 mL) and water (10 mL) was added lithium hydroxide monohydrate (0.890 g, 37.1 mmol) at 0° C. The mixture was warmed to rt and stirred overnight. HCl (1 N) was added at 0° C. to adjust pH to 5. The mixture was diluted with water and the mixture was extracted with EtOAc (80 mL×3). The combined organic layers were washed with brine, dried over Na₂SO₄, filtered and concentrated to dryness to give 5-[2-[1-(2-trimethylsilylethoxymethyl)pyrazol-4-yl]ethynyl]thiazole-4-carboxylic acid (2.50 g, 96% yield) as a yellow solid. The crude was used in the next step without further purification. LCMS ESI (+) m/z 350.1 (M+H). ¹H NMR (400 MHz, DMSO-d₆) δ: 13.27 (br, 1H), 9.11 (s, 1H), 8.40 (s, 1H), 7.86 (s, 1H), 5.47 (s, 2H), 3.58 (t, J=8.0 Hz, 2H), 0.88 (t, J=8.0 Hz, 2H), 0.00 (s, 9H).

Step C: Preparation of 6-[1-(2-trimethylsilylethoxymethyl)pyrazol-4-yl]-5H-thiazolo[4,5-c]pyridin-4-one: A solution of 5-[2-[1-(2-trimethylsilylethoxymethyl)pyrazol-4-yl]ethynyl]thiazole-4-carboxylic acid (1.20 g, 3.43 mmol) in 7 N ammonia in methanol (20 mL) was stirred for 10 h at 90° C. The mixture was concentration to dryness. The residue was then purified by flash column chromatography on silica gel (DCM/MeOH=100/1 to 20/1) to give 6-[1-(2-trimethylsilylethoxymethyl)pyrazol-4-yl]-5H-thiazolo[4,5-c]pyridin-4-one (900 mg, 75% yield) as a light yellow solid. LCMS ESI (+) m/z 349.1 (M+H). ¹H NMR (400 MHz, CDCl₃) δ: 8.82 (s, 1H), 8.70 (s, 1H), 8.04 (s, 1H), 7.30 (s, 1H), 7.06 (s, 1H), 5.59 (s, 2H), 3.71 (t, J=8.0 Hz, 2H), 0.99 (t, J=8.0 Hz, 2H), 0.00 (s, 9H).

Step D: Preparation of 7-bromo-6-(1-((2-(trimethylsilyl)ethoxy)methyl)-1H-pyrazol-4-yl)thiazolo[4,5-c]pyridin-4(5H)-one: To a solution of 6-[1-(2-trimethylsilylethoxymethyl)pyrazol-4-yl]-5H-thiazolo[4,5-c]pyridin-4-one (900 mg, 2.58 mmol) in DMF (12 mL) was added NBS (505 mg, 2.84 mmol) at 0° C. The mixture was stirred at 0° C. for 1 h. To the above solution was added saturated aqueous sodium thiosulfate solution and water. The mixture was filtered. The filter cake was washed with water and dried to give crude product (950 mg), which was used in the next step without further purification. LCMS ESI (+) m/z 427.0 (M+H).

Step E: Preparation of 2-[[4-(7-bromo-4-methoxy-thiazolo[4,5-c]pyridin-6-yl)pyrazol-1-yl]methoxy]ethyl-trimethyl-silane: A solution of 7-bromo-6-[1-(2-trimethylsilylethoxymethyl)pyrazol-4-yl]-5H-thiazolo[4,5-c]pyridin-4-one (850 mg, 1.99 mmol), silver carbonate (548 mg, 1.99 mmol) and methyl iodide (0.12 mL, 1.99 mmol) in toluene (12 mL) was stirred for 10 h at 90° C. The mixture was filtered and washed with EtOAc. The filtrate was concentrated under vacuum and purified by column chromatography (petroleum ether/EtOAc=10/1) to give 2-[[4-(7-bromo-4-methoxy-thiazolo[4,5-c]pyridin-6-yl)pyrazol-1-yl]methoxy]ethyl-trimethyl-silane (320 mg, 36% yield) as a yellow oil. LCMS ESI (+) m/z 441.0 (M+H).

Step F: Preparation of 2-[[4-[7-(4-fluoro-2-methoxy-phenyl)-4-methoxy-thiazolo[4,5-c]pyridin-6-yl]pyrazol-1-yl]methoxy]ethyl-trimethyl-silane: A suspension of 2-[[4-(7-bromo-4-methoxy-thiazolo[4,5-c]pyridin-6-yl)pyrazol-1-yl]methoxy]ethyl-trimethyl-silane (240 mg, 0.544 mmol), 2-(4-fluoro-2-methoxy-phenyl)-4,4,5,5-tetramethyl-1,3,2-dioxaborolane (164 mg, 0.652 mmol), 2-[[4-(7-bromo-4-methoxy-thiazolo[4,5-c]pyridin-6-yl)pyrazol-1-yl]methoxy]ethyl-trimethyl-silane (240 mg, 0.544 mmol), tri-tert-butylphosphine tetrafluoroborate (315 mg, 1.09 mmol), 1,1′-bis(diphenylphosphino)ferrocenedichloro palladium(II) (40 mg, 0.054 mmol) and cesium carbonate (18 mg, 0.054 mmol) in 1,4-dioxane (4 mL) and water (1 mL) was stirred for 1 h at 90° C. under N₂ atmosphere. The mixture was diluted with water (30 mL) and the product was extracted with EtOAc (40 ml×3). The organic layers were washed with brine, separated and dried over Na₂SO₄ before concentration to dryness. The residue was then purified by flash column chromatography (petroleum ether/EA=4/1) to give 2-[[4-[7-(4-fluoro-2-methoxy-phenyl)-4-methoxy-thiazolo[4,5-c]pyridin-6-yl]pyrazol-1-yl]methoxy]ethyl-trimethyl-silane (200 mg, 76% yield) as a yellow oil. LCMS ESI (+) m/z 487.2 (M+H).

Step G: Preparation of 7-(4-fluoro-2-methoxy-phenyl)-4-methoxy-6-(1H-pyrazol-4-yl)thiazolo[4,5-c]pyridine: To a solution of 2-[[4-[7-(4-fluoro-2-methoxy-phenyl)-4-methoxy-thiazolo[4,5-c]pyridin-6-yl]pyrazol-1-yl]methoxy]ethyl-trimethyl-silane (250 mg, 0.514 mmol) in THF (3 mL) was added tetra-n-butylammonium fluoride (3.00 mL, 0.514 mmol). The mixture was warmed to 50° C. and stirred for 4 h. The mixture was diluted with water (45 mL) and extracted with EtOAc (40 ml×3). The organic layers were washed with brine, separated, and dried over Na₂SO₄ before concentration to dryness. The crude was then purified by preparative TLC (MeOH/DCM=1/20) to give 7-(4-fluoro-2-methoxy-phenyl)-4-methoxy-6-(1H-pyrazol-4-yl)thiazolo[4,5-c]pyridine (165 mg, 90% yield) as yellow oil. LCMS ESI (+) m/z 357.1 (M+H).

Step H: Preparation of 7-(4-fluoro-2-methoxyphenyl)-6-(1H-pyrazol-4-yl)thiazolo[4,5-c]pyridin-4(5H)-one: To a suspension of 7-(4-fluoro-2-methoxy-phenyl)-4-methoxy-6-(1H-pyrazol-4-yl)thiazolo[4,5-c]pyridine (160 mg, 0.449 mmol) and sodium iodide (67 mg, 0.45 mmol) in ACN (3 mL) was added trimethylchlorosilane (0.057 mL, 0.45 mmol) at 0° C. The mixture was warmed to rt and stirred for 10 h. The mixture was diluted with water (45 mL) and extracted with EtOAc (40 ml×3). The organic layers were washed with brine and dried over Na₂SO₄ before concentration to dryness to give crude 7-(4-fluoro-2-methoxyphenyl)-6-(1H-pyrazol-4-yl)thiazolo[4,5-c]pyridin-4(5H)-one (140 mg), which was used in the next step without further purification. LCMS ESI (+) m/z 343.1 (M+H).

Step I: Preparation of 4-chloro-7-(4-fluoro-2-methoxy-phenyl)-6-(1H-pyrazol-4-yl)thiazolo[4,5-c]pyridine: A solution of 7-(4-fluoro-2-methoxy-phenyl)-6-(1H-pyrazol-4-yl)-5H-thiazolo[4,5-c]pyridin-4-one (140 mg, 0.409 mmol) in phosphorus oxychloride (6.00 mL, 64.4 mmol) was stirred for 6 h at 100° C. The mixture was concentrated under vacuum. The residue was diluted with EtOAc and poured into ice water (200 mL). Saturated sodium bicarbonate solution was added to adjust pH to 9. The product was extracted with EtOAc (150 ml×3) and the organic layers were separated and dried over Na₂SO₄ before concentration to dryness. The residue was then purified by preparative TLC (MeOH/DCM=1/20) to give 4-chloro-7-(4-fluoro-2-methoxy-phenyl)-6-(1H-pyrazol-4-yl)thiazolo[4,5-c]pyridine (100 mg, 68% yield) as a light yellow solid. LCMS ESI (+) m/z 361.0 (M+H).

Step J: Preparation of tert-butyl 3-[4-[4-chloro-7-(4-fluoro-2-methoxy-phenyl)thiazolo[4,5-c]pyridin-6-yl]pyrazol-1-yl]azetidine-1-carboxylate: A solution of 4-chloro-7-(4-fluoro-2-methoxy-phenyl)-6-(1H-pyrazol-4-yl)thiazolo[4,5-c]pyridine (20 mg, 0.055 mmol), cesium carbonate (18 mg, 0.055 mmol) and tert-butyl 3-iodoazetidine-1-carboxylate (16 mg, 0.055 mmol) in DMF (2 mL) was stirred at 80° C. for 2 h. The mixture was diluted with water (45 mL) and the product was extracted with EtOAc (40 mL×3). The organic layers were washed with brine, separated, and dried over Na₂SO₄ before concentration to dryness. The crude was then purified by preparative TLC (MeOH/DCM=1/20) to give tert-butyl 3-[4-[4-chloro-7-(4-fluoro-2-methoxy-phenyl)thiazolo[4,5-c]pyridin-6-yl]pyrazol-1-yl]azetidine-1-carboxylate (24 mg, 84% yield) as yellow oil. LCMS ESI (+) m/z 516.1 (M+H).

Step K: Preparation of 6-(1-(azetidin-3-yl)-1H-pyrazol-4-yl)-4-chloro-7-(4-fluoro-2-methoxyphenyl)thiazolo[4,5-c]pyridine: To a solution of tert-butyl 3-[4-[4-chloro-7-(4-fluoro-2-methoxy-phenyl)thiazolo[4,5-c]pyridin-6-yl]pyrazol-1-yl]azetidine-1-carboxylate (24 mg, 0.047 mmol) in DCM (3 mL) was added TFA (1 mL). The mixture was stirred at ambient temperature for 1 h. The reaction was concentrated under reduced pressure to give crude 6-(1-(azetidin-3-yl)-1H-pyrazol-4-yl)-4-chloro-7-(4-fluoro-2-methoxyphenyl)thiazolo[4,5-c]pyridine (26 mg), which was used in the next step without further purification. LCMS ESI (+) m/z 416.1 (M+H).

Step L: Preparation of 1-[3-[4-[4-chloro-7-(4-fluoro-2-methoxy-phenyl)thiazolo[4,5-c]pyridin-6-yl]pyrazol-1-yl]azetidin-1-yl]prop-2-en-1-one: To a solution of 6-[1-(azetidin-3-yl)pyrazol-4-yl]-4-chloro-7-(4-fluoro-2-methoxy-phenyl)thiazolo[4,5-c]pyridine (19 mg, 0.046 mmol) in dry DCM (3 mL) was added triethylamine (0.019 mL, 0.14 mmol). The mixture was cooled to −60° C. Acrylic anhydride (7.5 mg, 0.059 mmol) in dry DCM (1 mL) was added. Then the mixture was warmed to rt and stirred for 10 min. The mixture was quenched with saturated sodium bicarbonate solution (2 mL), diluted with water (10 mL) and extracted with EtOAc (20 mL×3). The combined organic layers were washed with brine (10 mL), dried over Na₂SO₄, filtered and concentrated under reduced pressure. The residue was purified by preparative TLC (MeOH/DCM=1/20) to give 1-[3-[4-[4-chloro-7-(4-fluoro-2-methoxy-phenyl)thiazolo[4,5-c]pyridin-6-yl]pyrazol-1-yl]azetidin-1-yl]prop-2-en-1-one (18 mg, 84% yield) as colorless oil. LCMS ESI (+) m/z 470.1 (M+H).

Step M: Preparation of tert-butyl 6-[7-(4-fluoro-2-methoxy-phenyl)-6-[1-(1-prop-2-enoylazetidin-3-yl)pyrazol-4-yl]thiazolo[4,5-c]pyridin-4-yl]-3,4-dihydro-1H-isoquinoline-2-carboxylate: A mixture of 1-[3-[4-[4-chloro-7-(4-fluoro-2-methoxy-phenyl)thiazolo[4,5-c]pyridin-6-yl]pyrazol-1-yl]azetidin-1-yl]prop-2-en-1-one (18 mg, 0.038 mmol), tert-butyl 6-(4,4,5,5-tetramethyl-1,3,2-dioxaborolan-2-yl)-3,4-dihydro-1H-isoquinoline-2-carboxylate (18 mg, 0.050 mmol), sodium carbonate (8.1 mg, 0.077 mmol) and tetrakis(triphenylphosphine) palladium(0) (4.4 mg, 0.0038 mmol) in 1,4-dioxane (1.2 mL) and water (0.3 mL) was stirred at 100° C. for 4 h under N₂ atmosphere. The mixture was diluted with water (30 mL) and extracted with EtOAc (40 mL×3). The organic layers were washed with brine, separated and dried over Na₂SO₄ before concentration to dryness. The crude was then purified by preparative TLC (MeOH/DCM=1/20) to give tert-butyl 6-[7-(4-fluoro-2-methoxy-phenyl)-6-[1-(1-prop-2-enoylazetidin-3-yl)pyrazol-4-yl]thiazolo[4,5-c]pyridin-4-yl]-3,4-dihydro-1H-isoquinoline-2-carboxylate (18 mg, 70% yield) as colorless oil. LCMS ESI (+) m/z 667.2 (M+H).

Step N: Preparation of 1-[3-[4-[7-(4-fluoro-2-methoxy-phenyl)-4-(1,2,3,4-tetrahydroisoquinolin-6-yl)thiazolo[4,5-c]pyridin-6-yl]pyrazol-1-yl]azetidin-1-yl]prop-2-en-1-one: To a solution of tert-butyl 6-[7-(4-fluoro-2-methoxy-phenyl)-6-[1-(1-prop-2-enoylazetidin-3-yl)pyrazol-4-yl]thiazolo[4,5-c]pyridin-4-yl]-3,4-dihydro-1H-isoquinoline-2-carboxylate (20 mg, 0.030 mmol) in DCM (3 mL) was added TFA (1 mL). The mixture was stirred at ambient temperature for 1 h. The mixture was concentrated under reduced pressure, and the residue was purified by reverse phase preparative HPLC to give 1-[3-[4-[7-(4-fluoro-2-methoxy-phenyl)-4-(1,2,3,4-tetrahydroisoquinolin-6-yl)thiazolo[4,5-c]pyridin-6-yl]pyrazol-1-yl]azetidin-1-yl]prop-2-en-1-one (8.5 mg, 50% yield) as a trifluoroacetic acid salt. LCMS ESI (+) m/z 567.2 (M+H). ¹H NMR (400 MHz, CD₃OD) δ 9.27 (s, 1H), 8.51-8.53 (m, 2H), 7.74 (s, 1H), 7.55 (s, 1H), 7.48 (d, J=8.0 Hz, 1H), 7.32-7.36 (m, 1H), 7.09 (dd, J=11.2, 2.2 Hz, 1H), 6.92-6.97 (m, 1H), 6.28-6.43 (m, 2H), 5.80 (dd, J=9.6, 2.0 Hz, 1H), 5.24-5.31 (m, 1H), 4.76 (t, J=8.8 Hz, 1H), 4.58-4.62 (m, 1H), 4.50-4.54 (m, 3H), 4.34-4.36 (m, 1H), 3.72 (s, 3H), 3.63 (t, J=6.4 Hz, 2H), 3.27-3.29 (m, 2H).

### Synthetic Example 18: Synthesis of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,8-dihydro-5H-[1,2,4]triazolo[1,5-a]pyrazin-7-yl]prop-2-en-1-one (Compound 105)

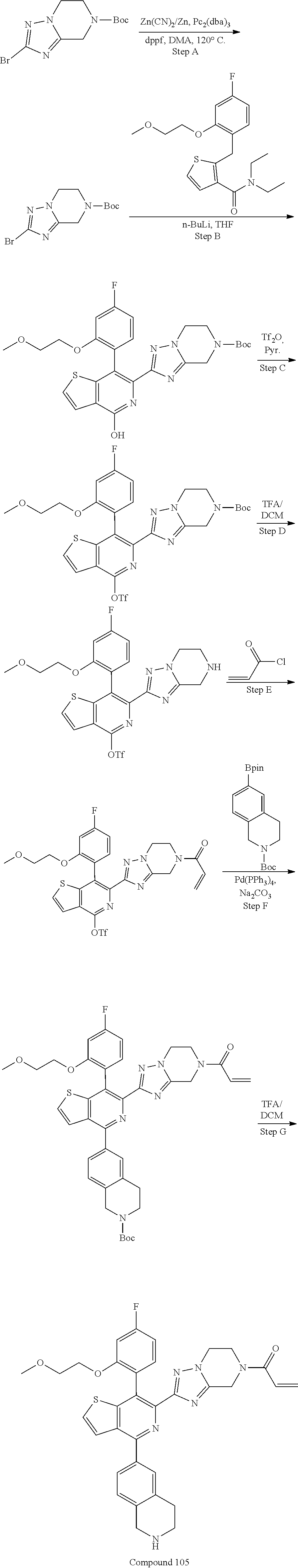

Step A: Preparation of tert-butyl 2-cyano-6,8-dihydro-5H-[1,2,4]triazolo[1,5-a]pyrazine-7-carboxylate: To a suspension of tert-butyl 2-bromo-6,8-dihydro-5H-[1,2,4]triazolo[1,5-a]pyrazine-7-carboxylate (300 mg, 0.990 mmol), dicyanozinc (139 mg, 1.19 mmol), zinc (7.8 mg, 0.12 mmol), 1,1′-bis(diphenylphosphino)ferrocene (110 mg, 0.198 mmol) in DMA (5 mL) was added tris(dibenzylideneacetone)dipalladium (91 mg, 0.099 mmol) under argon. The resulting mixture was stirred at 120° C. for 3 h. The reaction was diluted with EtOAc and filtered. The filtrate was concentrated to dryness and diluted with ACN (3 mL). The crude was then purified by reserve-phase preparative HPLC to give tert-butyl 2-cyano-6,8-dihydro-5H-[1,2,4]triazolo[1,5-a]pyrazine-7-carboxylate (188 mg, 76% yield) as light-yellow solid. ¹H NMR (400 MHz, CDCl₃) δ 4.79 (s, 2H), 4.27 (t, J=5.2 Hz, 2H), 3.98 (t, J=5.2 Hz, 2H), 1.50 (s, 9H).

Step B: Preparation of tert-butyl 2-[7-[4-fluoro-2-(2-methoxyethoxy)phenyl]-4-hydroxy-thieno[3,2-c]pyridin-6-yl]-6,8-dihydro-5H-[1,2,4]triazolo[1,5-a]pyrazine-7-carboxylate: To a solution of N,N-diethyl-2-[[4-fluoro-2-(2-methoxyethoxy)phenyl]methyl]thiophene-3-carboxamide (130 mg, 0.356 mmol) in THF (3 mL) was added n-butyllithium (0.15 mL, 2.5 M in THF, 0.375 mmol) at −60° C. The mixture was stirred at this temperature for 15 min, then tert-butyl 2-cyano-6,8-dihydro-5H-[1,2,4]triazolo[1,5-a]pyrazine-7-carboxylate (60 mg, 0.241 mmol) was added. The mixture was poured into water. 1 N aqueous HCl was added to adjust pH to 5. The product was extracted with EtOAc, dried over anhydrous Na₂SO₄, filtered and concentrated. The residue was purified by preparative TLC (DCM/MeOH: 20/1) to give tert-butyl 2-[7-[4-fluoro-2-(2-methoxyethoxy)phenyl]-4-hydroxy-thieno[3,2-c]pyridin-6-yl]-6,8-dihydro-5H-[1,2,4]triazolo[1,5-a]pyrazine-7-carboxylate (18 mg, 14% yield). LCMS ESI (+) m/z 542.2 (M+H).

Step C: Preparation of [7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]trifluoromethanesulfonate: To a solution of tert-butyl 2-[7-[4-fluoro-2-(2-methoxyethoxy)phenyl]-4-hydroxy-thieno[3,2-c]pyridin-6-yl]-6,8-dihydro-5H-[1,2,4]triazolo[1,5-a]pyrazine-7-carboxylate (40 mg, 0.035 mmol) in DCM (3 mL) was added triethylamine (0.041 mL, 0.30 mmol), followed by acryloyl chloride (0.0066 mL, 0.081 mmol) at −60° C. The mixture was stirred at −60° C. for 15 min. The mixture was poured into NaHCO₃ aqueous solution. The product was extracted with EtOAc, dried over anhydrous Na₂SO₄, concentrated and purified by preparative TLC (EtOAc/petroleum ether: 1/1) to give [7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]trifluoromethanesulfonate (16 mg, 73% yield). LCMS ESI (+) m/z 674.1 (M+H). ¹H NMR (400 MHz, CDCl₃) δ:7.67 (d, J=5.6 Hz, 1H), 7.55 (d, J=5.6 Hz, 1H), 7.28-7.30 (m, 1H), 6.77 (dt, J=8.3, 2.0 Hz, 1H), 6.72 (dd, J=10.7, 2.0 Hz, 1H), 4.67 (s, 2H), 4.10 (t, J=5.4 Hz, 2H), 3.93-3.99 (m, 1H), 3.83-3.91 (m, 2H), 3.77-3.82 (m, 1H), 3.36 (t, J=5.4 Hz, 2H), 3.07 (s, 3H), 1.49 (s, 9H).

Step D: Preparation of 7-(4-fluoro-2-(2-methoxyethoxy)phenyl)-6-(5,6,7,8-tetrahydro-[1,2,4]triazolo[1,5-a]pyrazin-2-yl)thieno[3,2-c]pyridin-4-yl trifluoromethanesulfonate: To a solution of tert-butyl 2-[7-[4-fluoro-2-(2-methoxyethoxy)phenyl]-4-(trifluoromethylsulfonyloxy)thieno[3,2-c]pyridin-6-yl]-6,8-dihydro-5H-[1,2,4]triazolo[1,5-a]pyrazine-7-carboxylate (24 mg, 0.036 mmol) in DCM (2 mL) was added TFA (0.5 mL). The mixture was stirred at 25° C. for 1 h. The mixture was concentrated in vacuo to give crude [7-[4-fluoro-2-(2-methoxyethoxy)phenyl]-6-(5,6,7,8-tetrahydro-[1,2,4]triazolo[1,5-a]pyrazin-2-yl)thieno[3,2-c]pyridin-4-yl]trifluoromethanesulfonate (40 mg) as a trifluoroacetic acid salt which was used in the next step directly. LCMS ESI (+) m/z 574.1 (M+H).

Step E: Preparation of [7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]trifluoromethanesulfonate: To a solution of [7-[4-fluoro-2-(2-methoxyethoxy)phenyl]-6-(5,6,7,8-tetrahydro-[1,2,4]triazolo[1,5-a]pyrazin-2-yl)thieno[3,2-c]pyridin-4-yl]trifluoromethanesulfonate; 2,2,2-trifluoroacetic acid (40 mg, 0.035 mmol) in DCM (3 mL) was added triethylamine (0.041 mL, 0.30 mmol), followed by acryloyl chloride (0.0066 mL, 0.081 mmol) at −60° C. The mixture was stirred at −60° C. for 15 min. The mixture was poured into NaHCO₃ aqueous solution. The product was extracted with EtOAc, dried over anhydrous Na₂SO₄, concentrated and purified by preparative TLC (EtOAc/petroleum ether: 1/1) to give [7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]trifluoromethanesulfonate (16 mg, 73% yield). LCMS ESI (+) m/z 628.1 (M+H). ¹H NMR (400 MHz, CDCl₃) δ 7.67 (d, J=5.6 Hz, 1H), 7.55 (d, J=5.6 Hz, 1H), 7.28-7.30 (m, 1H), 6.78 (dt, J=8.3, 2.0 Hz, 1H), 6.72 (dd, J=10.7, 2.0 Hz, 1H), 6.55-6.60 (m, 1H), 6.40 (d, J=16.7 Hz, 1H), 5.84 (d, J=10.5 Hz, 1H), 4.85 (s, 2H), 4.0-4.28 (m, 4H), 3.91-3.99 (m, 1H), 3.80-3.85 (m, 1H), 3.36 (t, J=4.6 Hz, 2H), 3.05 (s, 3H).

Step F: Preparation of tert-butyl 6-[7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]-3,4-dihydro-1H-isoquinoline-2-carboxylate: To a solution of [7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]trifluoromethanesulfonate (16 mg, 0.026 mmol), sodium carbonate (6.0 mg, 0.057 mmol), and tert-butyl 6-(4,4,5,5-tetramethyl-1,3,2-dioxaborolan-2-yl)-3,4-dihydro-1H-isoquinoline-2-carboxylate (16 mg, 0.045 mmol) in 1,4-dioxane (2 mL) and water (0.2 mL) was added tetrakis(triphenylphosphine)palladium(0) (3.0 mg, 0.0026 mmol). The mixture was stirred at 100° C. for 2 h under Ar. The mixture was concentrated and purified by preparative TLC (EtOAc) to give tert-butyl 6-[7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]-3,4-dihydro-1H-isoquinoline-2-carboxylate (14 mg, 77% yield).

Step G: Preparation of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,8-dihydro-5H-[1,2,4]triazolo[1,5-a]pyrazin-7-yl]prop-2-en-1-one: To a solution of tert-butyl 6-[7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]-3,4-dihydro-1H-isoquinoline-2-carboxylate (14 mg, 0.020 mmol) in DCM (2 mL) was added TFA (0.5 mL). The mixture was stirred at 25° C. for 1 h. The mixture was concentrated and purified by reverse phase preparative HPLC to give 1-[2-[7-[4-fluoro-2-(2-methoxyethoxy)phenyl]-4-(1,2,3,4-tetrahydroisoquinolin-6-yl)thieno[3,2-c]pyridin-6-yl]-6,8-dihydro-5H-[1,2,4]triazolo[1,5-a]pyrazin-7-yl]prop-2-en-1-one (11 mg, 88% yield) as a bis trifluoroacetic acid salt. LCMS ESI (+) m/z 611.2 (M+H). ¹H NMR (400 MHz, CD₃OD) δ 7.92 (s, 1H), 7.90 (d, J=8.0 Hz, 1H), 7.85 (d, J=5.6 Hz, 1H), 7.70 (d, J=5.6 Hz, 1H), 7.45 (d, J=8.0 Hz, 1H), 7.32-7.35 (m, 1H), 6.75-6.92 (m, 3H), 6.30 (d, J=16.8 Hz, 1H), 5.84 (dd, J=10.6, 1.7 Hz, 1H), 4.90-5.05 (m, 2H), 4.50 (s, 2H), 4.09-4.20 (m, 4H), 3.96-4.04 (m, 1H), 3.82-3.89 (m, 1H), 3.60 (t, J=6.4 Hz, 2H), 3.41 (t, J=4.8 Hz, 2H), 3.20-3.31 (m, 2H), 3.06 (s, 3H).

### Synthetic Example 19: Synthesis of 5-[7-(4-fluoro-2-methoxy-phenyl)-4-(1,2,3,4-tetrahydroisoquinolin-6-yl)thieno[3,2-c]pyridin-6-yl]-1-(1-prop-2-enoylazetidin-3-yl)pyridin-2-one (Compound 110)

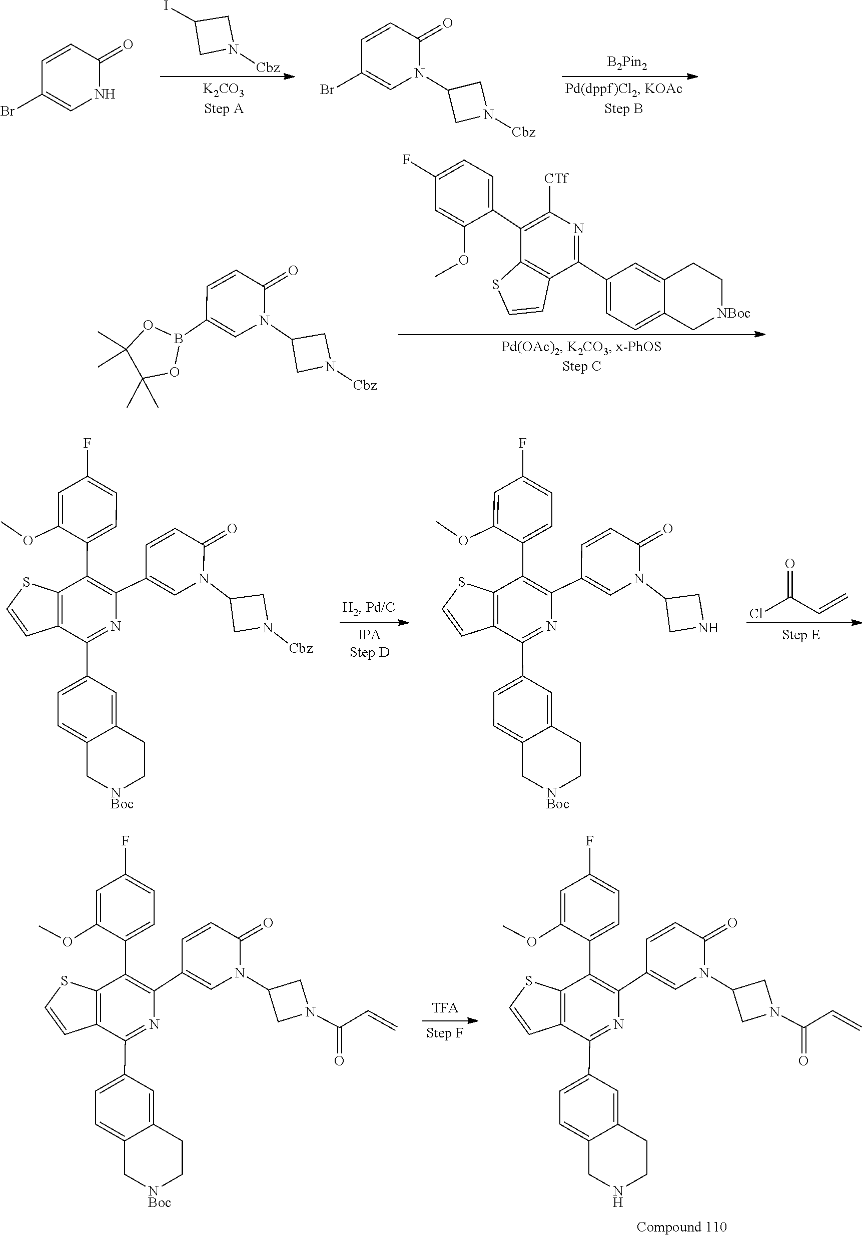

Step A: Preparation of benzyl 3-(5-bromo-2-oxo-1-pyridyl)azetidine-1-carboxylate: A mixture of benzyl 3-iodoazetidine-1-carboxylate (1.00 g, 3.15 mmol), 5-bromo-1H-pyridin-2-one (0.658 g, 3.78 mmol) and potassium carbonate (0.870 g, 6.31 mmol) in DMF (8 mL) was added at 80° C. for 6 hours. The mixture was diluted with EtOAc, washed with brine, dried over Na₂SO₄ and concentrated. The residue was purified by silica gel column (petroleum ether:EtOAc=5:1 to 3:1) to give benzyl 3-(5-bromo-2-oxo-1-pyridyl)azetidine-1-carboxylate (910 mg, 79% yield). LCMS ESI (+) m/z 363.1 (M+H).

Step B: Preparation of benzyl 3-[2-oxo-5-(4,4,5,5-tetramethyl-1,3,2-dioxaborolan-2-yl)-1-pyridyl]azetidine-1-carboxylate: A mixture of benzyl 3-(5-bromo-2-oxo-1-pyridyl)azetidine-1-carboxylate (910 mg, 2.51 mmol), bis(pinacolato)diboron (763 mg, 3.01 mmol), potassium acetate (492 mg, 5.01 mmol) and 1,1′-bis(diphenylphosphino)ferrocenedichloro palladium(II) dichloromethane complex (183 mg, 0.251 mmol) in 1,4-dioxane (20 mL) was stirred at 90° C. under N₂ for 12 hours. The mixture was concentrated and purified by silica gel column (petroleum ether:EtOAc=10:1 to 3:1) to give benzyl 3-[2-oxo-5-(4,4,5,5-tetramethyl-1,3,2-dioxaborolan-2-yl)-1-pyridyl]azetidine-1-carboxylate (710 mg, 69% yield). LCMS ESI (+) m/z 411.3 (M+H).

Step C: Preparation of tert-butyl 6-[6-[1-(1-benzyloxycarbonylazetidin-3-yl)-6-oxo-3-pyridyl]-7-(4-fluoro-2-methoxy-phenyl)thieno[3,2-c]pyridin-4-yl]-3,4-dihydro-1H-isoquinoline-2-carboxylate: A mixture of tert-butyl 6-[7-(4-fluoro-2-methoxy-phenyl)-6-(trifluoromethylsulfonyloxy)thieno[3,2-c]pyridin-4-yl]-3,4-dihydro-1H-isoquinoline-2-carboxylate (prepared as described in Synthetic Example 10 Steps I to L, substituting 4-fluoro-2-methoxy-phenyl)boronic acid for [4-fluoro-2-(2-methoxyethoxy)phenyl]boronic acid in Step I, 80 mg, 0.125 mmol), benzyl 3-[2-oxo-5-(4,4,5,5-tetramethyl-1,3,2-dioxaborolan-2-yl)-1-pyridyl]azetidine-1-carboxylate (82 mg, 0.20 mmol), palladium(II) acetate (2.8 mg, 0.013 mmol), X-PHOS (12 mg, 0.025 mmol) and K₂CO₃ (6.0 mg, 0.25 mmol) in acetonitrile (2 mL)/water (0.2 mL) was stirred at 100° C. for 2 hours under N₂. The mixture was filtered and concentrated, and the residue was purified by preparative TLC (Petroleum ether:EtOAc=3:2) to give tert-butyl 6-[6-[1-(1-benzyloxycarbonylazetidin-3-yl)-6-oxo-3-pyridyl]-7-(4-fluoro-2-methoxy-phenyl)thieno[3,2-c]pyridin-4-yl]-3,4-dihydro-1H-isoquinoline-2-carboxylate (66 mg, 68% yield). LCMS ESI (+) m/z 773.6 (M+H).

Step D: Preparation of 7-(4-fluoro-2-(2-methoxyethoxy)phenyl)-6-(5,6,7,8-tetrahydro-[1,2,4]triazolo[1,5-a]pyrazin-2-yl)thieno[3,2-c]pyridin-4-yl trifluoromethanesulfonate: To a solution of tert-butyl 2-[7-[4-fluoro-2-(2-methoxyethoxy)phenyl]-4-(trifluoromethylsulfonyloxy)thieno[3,2-c]pyridin-6-yl]-6,8-dihydro-5H-[1,2,4]triazolo[1,5-a]pyrazine-7-carboxylate (24 mg, 0.036 mmol) in DCM (2 mL) was added TFA (0.5 mL). The mixture was stirred at 25° C. for 1 h. The mixture was concentrated in vacuo to give crude [7-[4-fluoro-2-(2-methoxyethoxy)phenyl]-6-(5,6,7,8-tetrahydro-[1,2,4]triazolo[1,5-a]pyrazin-2-yl)thieno[3,2-c]pyridin-4-yl]trifluoromethanesulfonate (40 mg) as a trifluoroacetic acid salt which was used in the next step directly. LCMS ESI (+) m/z 574.1 (M+H).

Step E: Preparation of [7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]trifluoromethanesulfonate: To a solution of [7-[4-fluoro-2-(2-methoxyethoxy)phenyl]-6-(5,6,7,8-tetrahydro-[1,2,4]triazolo[1,5-a]pyrazin-2-yl)thieno[3,2-c]pyridin-4-yl]trifluoromethanesulfonate; 2,2,2-trifluoroacetic acid (40 mg, 0.035 mmol) in DCM (3 mL) was added triethylamine (0.041 mL, 0.30 mmol), followed by acryloyl chloride (0.0066 mL, 0.081 mmol) at −60° C. The mixture was stirred at −60° C. for 15 min. The mixture was poured into NaHCO₃ aqueous solution. The product was extracted with EtOAc, dried over anhydrous Na₂SO₄, concentrated and purified by preparative TLC (EtOAc/petroleum ether: 1/1) to give [7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]trifluoromethanesulfonate (16 mg, 73% yield). LCMS ESI (+) m/z 628.1 (M+H). ¹H NMR (400 MHz, CDCl₃) δ 7.67 (d, J=5.6 Hz, 1H), 7.55 (d, J=5.6 Hz, 1H), 7.28-7.30 (m, 1l), 6.78 (dt, J=8.3, 2.0 Hz, 1H), 6.72 (dd, J=10.7, 2.0 Hz, 1H), 6.55-6.60 (m, 1H), 6.40 (d, J=16.7 Hz, 1H), 5.84 (d, J=10.5 Hz, 1H), 4.85 (s, 2H), 4.0-4.28 (m, 4H), 3.91-3.99 (m, 1H), 3.80-3.85 (m, 1H), 3.36 (t, J=4.6 Hz, 2H), 3.05 (s, 3H).

Step F: Preparation of tert-butyl 6-[7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]-3,4-dihydro-1H-isoquinoline-2-carboxylate: To a solution of [7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]trifluoromethanesulfonate (16 mg, 0.026 mmol), sodium carbonate (6.0 mg, 0.057 mmol), and tert-butyl 6-(4,4,5,5-tetramethyl-1,3,2-dioxaborolan-2-yl)-3,4-dihydro-1H-isoquinoline-2-carboxylate (16 mg, 0.045 mmol) in 1,4-dioxane (2 mL) and water (0.2 mL) was added tetrakis(triphenylphosphine)palladium(0) (3.0 mg, 0.0026 mmol). The mixture was stirred at 100° C. for 2 h under Ar. The mixture was concentrated and purified by preparative TLC (EtOAc) to give tert-butyl 6-[7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]-3,4-dihydro-1H-isoquinoline-2-carboxylate (14 mg, 77% yield).

Step G: Preparation of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,8-dihydro-5H-[1,2,4]triazolo[1,5-a]pyrazin-7-yl]prop-2-en-1-one: To a solution of tert-butyl 6-[7-[4-fluoro-2-(2-methoxyethoxy)phenyl]-6-(7-prop-2-enoyl-6,8-dihydro-5H-[1,2,4]triazolo[1,5-a]pyrazin-2-yl)thieno[3,2-c]pyridin-4-yl]-3,4-dihydro-1H-isoquinoline-2-carboxylate (14 mg, 0.020 mmol) in DCM (2 mL) was added TFA (0.5 mL). The mixture was stirred at 25° C. for 1 h. The mixture was concentrated and purified by reverse phase preparative HPLC to give 1-[2-[7-[4-fluoro-2-(2-methoxyethoxy)phenyl]-4-(1,2,3,4-tetrahydroisoquinolin-6-yl)thieno[3,2-c]pyridin-6-yl]-6,8-dihydro-5H-[1,2,4]triazolo[1,5-a]pyrazin-7-yl]prop-2-en-1-one (11 mg, 88% yield) as a bis trifluoroacetic acid salt. LCMS ESI (+) m/z 611.2 (M+H). ¹H NMR (400 MHz, CD₃OD) δ 7.92 (s, 1H), 7.90 (d, J=8.0 Hz, 1H), 7.85 (d, J=5.6 Hz, 1H), 7.70 (d, J=5.6 Hz, 1H), 7.45 (d, J=8.0 Hz, 1H), 7.32-7.35 (m, 1H), 6.75-6.92 (m, 3H), 6.30 (d, J=16.8 Hz, 1H), 5.84 (dd, J=10.6, 1.7 Hz, 1H), 4.90-5.05 (m, 2H), 4.50 (s, 2H), 4.09-4.20 (m, 4H), 3.96-4.04 (m, 1H), 3.82-3.89 (m, 1H), 3.60 (t, J=6.4 Hz, 2H), 3.41 (t, J=4.8 Hz, 2H), 3.20-3.31 (m, 2H), 3.06 (s, 3H).

### Synthetic Example 20: Synthesis of 1-[2-[7-(4-fluoro-2-methoxy-phenyl)-4-(1,2,3,4-tetrahydroisoquinolin-6-yl)thieno[3,2-c]pyridin-6-yl]-6,7-dihydro-4H-oxazolo[4,5-c]pyridin-5-yl]prop-2-en-1-one (Compound 112)

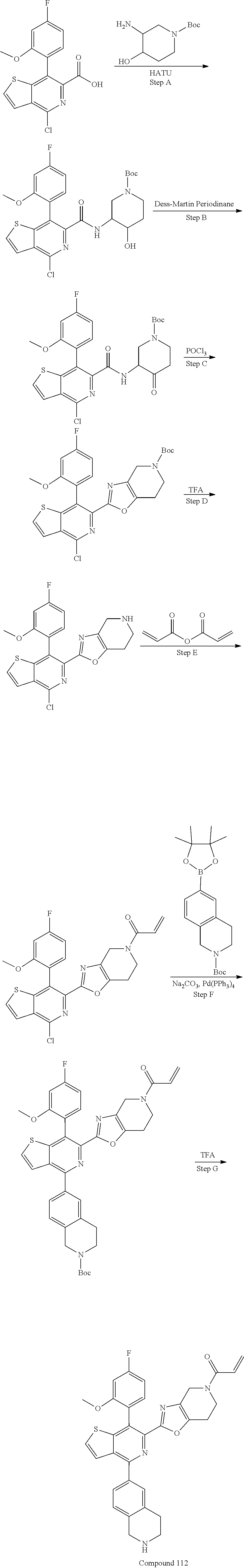

Step A: Preparation of tert-butyl 3-[[4-chloro-7-(4-fluoro-2-methoxy-phenyl)thieno[3,2-c]pyridine-6-carbonyl]amino]-4-hydroxy-piperidine-1-carboxylate: To a solution of 4-chloro-7-(4-fluoro-2-methoxy-phenyl)thieno[3,2-c]pyridine-6-carboxylic acid (20 mg, 0.059 mmol) in DCM (5 mL) were added triethylamine (0.011 mL, 0.077 mmol), EDCI (15 mg, 0.077 mmol) and HOBt (10 mg, 0.077 mmol). The mixture was stirred at 25° C. for 10 min. tert-Butyl 3-amino-4-hydroxy-piperidine-1-carboxylate (12 mg, 0.053 mmol) was added. The resulting mixture was stirred at ambient temperature for 2 h. The mixture was poured into water and extracted with EtOAc. The combined organic layers were washed with brine, dried over Na₂SO₄ and concentrated. The residue was purified by preparative TLC (petroleum ether:EtOAc 2:1) to give tert-butyl 3-[[4-chloro-7-(4-fluoro-2-methoxy-phenyl)thieno[3,2-c]pyridine-6-carbonyl]amino]-4-hydroxy-piperidine-1-carboxylate (18 mg, 57% yield). LCMS ESI (−) m/z 534 (M−H).

Step B: Preparation of tert-butyl 3-[[4-chloro-7-(4-fluoro-2-methoxy-phenyl)thieno[3,2-c]pyridine-6-carbonyl]amino]-4-oxo-piperidine-1-carboxylate: To a solution of tert-butyl 3-[[4-chloro-7-(4-fluoro-2-methoxy-phenyl)thieno[3,2-c]pyridine-6-carbonyl]amino]-4-hydroxy-piperidine-1-carboxylate (20 mg, 0.037 mmol) in DCM (5 mL) was added Dess-Martin periodinane (32 mg, 0.075 mmol). The mixture was stirred at 25° C. for 1 h. Aqueous NaHSO₃ solution (5 mL) was added to quench the reaction. The mixture was poured into water and extracted with EtOAc. The combined organic layers were washed with brine, dried over Na₂SO₄ and concentrated. The residue was purified by preparative TLC (petroleum ether:EtOAc 3:1) to give tert-butyl 3-[[4-chloro-7-(4-fluoro-2-methoxy-phenyl)thieno[3,2-c]pyridine-6-carbonyl]amino]-4-oxo-piperidine-1-carboxylate (15 mg, 75% yield). LCMS ESI (+) m/z 534.1 (M+H).

Step C: Preparation of tert-butyl 2-[4-chloro-7-(4-fluoro-2-methoxy-phenyl)thieno[3,2-c]pyridin-6-yl]-6,7-dihydro-4H-oxazolo[4,5-c]pyridine-5-carboxylate: To a solution of tert-butyl 3-[[4-chloro-7-(4-fluoro-2-methoxy-phenyl)thieno[3,2-c]pyridine-6-carbonyl]amino]-4-oxo-piperidine-1-carboxylate (80 mg, 0.15 mmol) in 1,4-dioxane (5 mL) was added phosphorus oxychloride (0.13 mL, 1.4 mmol). The mixture was stirred at 80° C. for 24 h. The mixture was poured into NaHCO₃ (10 mL) aqueous solution, and di-tert-butyl dicarbonate (0.031 mL, 0.14 mmol) in MeOH (2 mL) was added. The mixture was stirred at ambient temperature for 0.5 h. The mixture was diluted with water and extracted with EtOAc. The combined organic layers were washed with brine solution, dried over Na₂SO₄ and concentrated. The residue was purified by column chromatography (petroleum ether:EtOAc 5:1) to give tert-butyl 2-[4-chloro-7-(4-fluoro-2-methoxy-phenyl)thieno[3,2-c]pyridin-6-yl]-6,7-dihydro-4H-oxazolo[4,5-c]pyridine-5-carboxylate (33 mg, 43% yield). LCMS ESI (+) m/z 516.2 (M+H).

Step D: Preparation of 2-[4-chloro-7-(4-fluoro-2-methoxy-phenyl)thieno[3,2-c]pyridin-6-yl]-4,5,6,7-tetrahydrooxazolo[4,5-c]pyridine: To a solution of tert-butyl 2-[4-chloro-7-(4-fluoro-2-methoxy-phenyl)thieno[3,2-c]pyridin-6-yl]-6,7-dihydro-4H-oxazolo[4,5-c]pyridine-5-carboxylate (33 mg, 0.064 mmol) in DCM (4 mL) was added trifluoroacetic acid (1.0 mL). The mixture was stirred at 25° C. for 0.5 h. The reaction was concentrated to dryness to give crude 2-[4-chloro-7-(4-fluoro-2-methoxy-phenyl)thieno[3,2-c]pyridin-6-yl]-4,5,6,7-tetrahydrooxazolo[4,5-c]pyridine (35 mg) as a trifluoroacetic acid salt which was used in the next step directly. LCMS ESI (+) m/z 416 (M+H).

Step E: Preparation of tert-butyl 6-[7-(4-fluoro-2-methoxy-phenyl)-6-(5-prop-2-enoyl-6,7-dihydro-4H-oxazolo[4,5-c]pyridin-2-yl)thieno[3,2-c]pyridin-4-yl]-3,4-dihydro-1H-isoquinoline-2-carboxylate: To a solution of 2-[4-chloro-7-(4-fluoro-2-methoxy-phenyl)thieno[3,2-c]pyridin-6-yl]-4,5,6,7-tetrahydrooxazolo[4,5-c]pyridine (20 mg, 0.048 mmol) and triethylamine (0.020 mL, 0.14 mmol) in DCM (4 mL) was added prop-2-enoyl prop-2-enoate (7.3 mg, 0.058 mmol) at −30° C. The mixture was stirred at −30° C. for 0.5 h. The mixture was poured into aqueous NaHCO₃ solution and extracted with EtOAc. The combined organic layers were washed with brine, dried over Na₂SO₄ and concentrated. The residue was purified by column chromatography (petroleum ether:EtOAc 2:1) to give tert-butyl 6-[7-(4-fluoro-2-methoxy-phenyl)-6-(5-prop-2-enoyl-6,7-dihydro-4H-oxazolo[4,5-c]pyridin-2-yl)thieno[3,2-c]pyridin-4-yl]-3,4-dihydro-1H-isoquinoline-2-carboxylate (15 mg, 70% yield). LCMS ESI (+) m/z 470 (M+H).

Step F: Preparation of give tert-butyl 6-[7-(4-fluoro-2-methoxy-phenyl)-6-(5-prop-2-enoyl-6,7-dihydro-4H-oxazolo[4,5-c]pyridin-2-yl)thieno[3,2-c]pyridin-4-yl]-3,4-dihydro-1H-isoquinoline-2-carboxylate: A mixture of 1-[2-[4-chloro-7-(4-fluoro-2-methoxy-phenyl)thieno[3,2-c]pyridin-6-yl]-6,7-dihydro-4H-oxazolo[4,5-c]pyridin-5-yl]prop-2-en-1-one (15 mg, 0.032 mmol), tert-butyl 6-(4,4,5,5-tetramethyl-1,3,2-dioxaborolan-2-yl)-3,4-dihydro-1H-isoquinoline-2-carboxylate (17 mg, 0.048 mmol), tetrakis(triphenylphosphine)palladium(0) (3.7 mg, 0.0032 mmol) and sodium carbonate (6.8 mg, 0.064 mmol) in 1,4-dioxane (3 mL) and water (0.9 mL) was stirred at 90° C. for 4 h. The mixture was concentrated and purified by column chromatography (petroleum ether:EtOAc 2:1) to give tert-butyl 6-[7-(4-fluoro-2-methoxy-phenyl)-6-(5-prop-2-enoyl-6,7-dihydro-4H-oxazolo[4,5-c]pyridin-2-yl)thieno[3,2-c]pyridin-4-yl]-3,4-dihydro-1H-isoquinoline-2-carboxylate (15 mg, 70% yield). LCMS ESI (+) m/z 667 (M+H).

Step G: Preparation of 1-[2-[7-(4-fluoro-2-methoxy-phenyl)-4-(1,2,3,4-tetrahydroisoquinolin-6-yl)thieno[3,2-c]pyridin-6-yl]-6,7-dihydro-4H-oxazolo[4,5-c]pyridin-5-yl]prop-2-en-1-one: To a solution of tert-butyl 6-[7-(4-fluoro-2-methoxy-phenyl)-6-(5-prop-2-enoyl-6,7-dihydro-4H-oxazolo[4,5-c]pyridin-2-yl)thieno[3,2-c]pyridin-4-yl]-3,4-dihydro-1H-isoquinoline-2-carboxylate (15 mg, 0.023 mmol) in DCM (4 mL) was added trifluoroacetic acid (1.0 mL). The mixture was stirred at 25° C. for 0.5 h. The mixture was concentrated and purified by reverse phase preparative HPLC to give 1-[2-[7-(4-fluoro-2-methoxy-phenyl)-4-(1,2,3,4-tetrahydroisoquinolin-6-yl)thieno[3,2-c]pyridin-6-yl]-6,7-dihydro-4H-oxazolo[4,5-c]pyridin-5-yl]prop-2-en-1-one (6.0 mg, 47% yield) as a bis trifluoroacetic acid salt. LCMS ESI (+) m/z 567.2 (M+H). ¹H NMR (400 MHz, CD₃OD) δ 7.88-7.89 (m, 3H), 7.71 (d, J=5.6 Hz, 1H), 7.37-7.48 (m, 2H), 6.91-6.95 (m, 1H), 6.74-6.88 (m, 2H), 6.23 (d, J=16.4 Hz, 1H), 5.79 (d, J=10.8 Hz, 1H), 4.57 (s, 2H), 4.49 (s, 2H), 3.93-3.98 (m, 2H), 3.58-3.61 (m, 5H), 2.70-2.73 (m, 2H).

### Synthetic Example 21: Synthesis of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,7-dihydro-4H-pyrazolo[1,5-a]pyrazin-5-yl]prop-2-en-1-one (Compound 115)

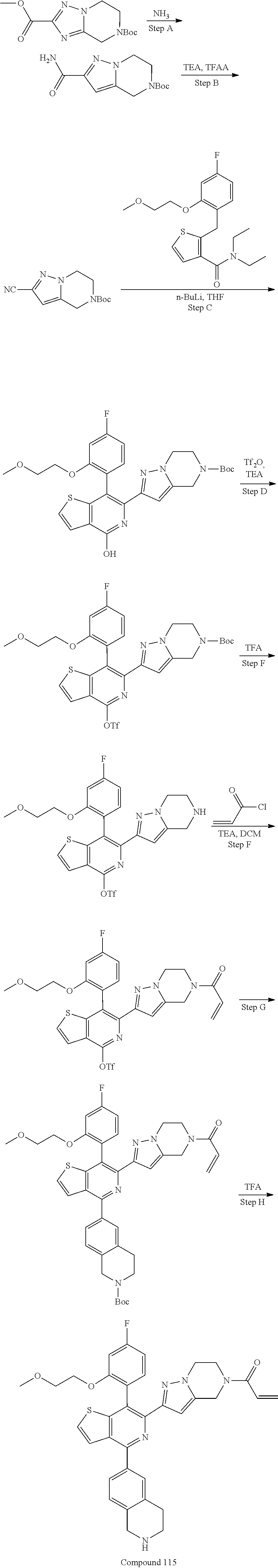

Step A: Preparation of tert-butyl 2-carbamoyl-6,7-dihydro-4H-pyrazolo[1,5-a]pyrazine-5-carboxylate: A solution of 5-(tert-butyl) 2-methyl 6,7-dihydropyrazolo[1,5-a]pyrazine-2,5(4H)-dicarboxylate (260 mg, 0.924 mmol) in 7 N ammonia solution in MeOH (5.0 mL) was stirred at 120° C. for 10 h. The mixture was concentrated in vacuo, and the residue was purified by preparative TLC (EtOAc/petroleum ether: 1/1) to give tert-butyl 2-carbamoyl-6,7-dihydro-4H-pyrazolo[1,5-a]pyrazine-5-carboxylate (180 mg, 73% yield). LCMS ESI (+) m/z 267.1 (M+H).

Step B: Preparation of tert-butyl 2-cyano-6,7-dihydro-4H-pyrazolo[1,5-a]pyrazine-5-carboxylate: To a solution of tert-butyl 2-carbamoyl-6,7-dihydro-4H-pyrazolo[1,5-a]pyrazine-5-carboxylate (160 mg, 0.601 mmol) and TEA (0.840 mL, 6.01 mmol) in DCE (10 mL) was added TFAA (379 mg, 1.80 mmol) at 0° C. The mixture was stirred at 25° C. for 3 h. The mixture was concentrated and diluted with water (10 mL). The product was extracted with EtOAc (10 mL). The organic phase was dried over anhydrous Na₂SO₄, filtered and concentrated. The residue was purified by preparative TLC (EtOAc/petroleum ether: 1/3) to give tert-butyl 2-cyano-6,7-dihydro-4H-pyrazolo[1,5-a]pyrazine-5-carboxylate (130 mg, 87% yield). LCMS ESI (+) m/z 294.2 (M+H). ¹H NMR (400 MHz, CDCl₃) δ: 6.49 (s, 1H), 4.77 (s, 2H), 4.24 (t, J=5.2 Hz, 2H), 3.92 (t, J=5.2 Hz, 2H), 1.50 (s, 9H).

Step C: Preparation of tert-butyl 2-(7-(4-fluoro-2-(2-methoxyethoxy)phenyl)-4-hydroxythieno[3,2-c]pyridin-6-yl)-6,7-dihydropyrazolo[1,5-a]pyrazine-5(4H)-carboxylate: To a solution of N,N-diethyl-2-(4-fluoro-2-(2-methoxyethoxy)benzyl)thiophene-3-carboxamide (51 mg, 0.14 mmol) in THF (3 mL) was added n-BuLi (0.060 mL, 0.15 mmol, 2.5 M in THF) at −60° C. The mixture was stirred at −60° C. for 15 min, then tert-butyl 2-cyano-6,7-dihydropyrazolo[1,5-a]pyrazine-5(4H)-carboxylate (30 mg, 0.12 mmol) was added, and the resulting mixture was stirred at 20° C. for additional 4 h. The reaction was quenched with water. 1 N aqueous HCl was added to adjust the pH to 4. The product was extracted with EtOAc, dried over Na₂SO₄, concentrated and purified by preparative TLC (DCM/MeOH: 20/1) to give tert-butyl 2-(7-(4-fluoro-2-(2-methoxyethoxy)phenyl)-4-hydroxythieno[3,2-c]pyridin-6-yl)-6,7-dihydropyrazolo[1,5-a]pyrazine-5(4H)-carboxylate (30 mg, 46% yield).

Step D: Preparation of tert-butyl 2-[7-[4-fluoro-2-(2-methoxyethoxy)phenyl]-4-(trifluoromethylsulfonyloxy)thieno[3,2-c]pyridin-6-yl]-6,7-dihydro-4H-pyrazolo[1,5-a]pyrazine-5-carboxylate: To a solution of tert-butyl 2-[7-[4-fluoro-2-(2-methoxyethoxy)phenyl]-4-hydroxy-thieno[3,2-c]pyridin-6-yl]-6,7-dihydro-4H-pyrazolo[1,5-a]pyrazine-5-carboxylate (30 mg, 0.056 mmol) and pyridine (0.043 mL, 0.53 mmol) in DCM (3 mL) was added trifluoromethanesulfonic anhydride (0.034 mL, 0.20 mmol). The mixture was stirred at 25° C. for 1 h. The mixture was concentrated, diluted with EtOAc, washed with brine, dried over anhydrous Na₂SO₄ and concentrated. The residue was purified by preparative TLC (EtOAc/PE: 1/2) to give tert-butyl 2-[7-[4-fluoro-2-(2-methoxyethoxy)phenyl]-4-(trifluoromethylsulfonyloxy)thieno[3,2-c]pyridin-6-yl]-6,7-dihydro-4H-pyrazolo[1,5-a]pyrazine-5-carboxylate (28 mg, 75% yield).

Step E: Preparation of [7-[4-fluoro-2-(2-methoxyethoxy)phenyl]-6-(4,5,6,7-tetrahydropyrazolo[1,5-a]pyrazin-2-yl)thieno[3,2-c]pyridin-4-yl]trifluoromethanesulfonate: To a solution of tert-butyl 2-[7-[4-fluoro-2-(2-methoxyethoxy)phenyl]-4-(trifluoromethylsulfonyloxy)thieno[3,2-c]pyridin-6-yl]-6,7-dihydro-4H-pyrazolo[1,5-a]pyrazine-5-carboxylate (28 mg, 0.042 mmol) in DCM (2 mL) was added TFA (0.5 mL). The mixture was stirred at 25° C. for 1 h. The mixture was concentrated in vacuo to give [7-[4-fluoro-2-(2-methoxyethoxy)phenyl]-6-(4,5,6,7-tetrahydropyrazolo[1,5-a]pyrazin-2-yl)thieno[3,2-c]pyridin-4-yl]trifluoromethanesulfonate (35 mg, 98% yield) as a trifluoroacetic acid salt, which was used in the next step directly. LCMS ESI (+) m/z 573.1 (M+H).

Step F: Preparation of [7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]trifluoromethanesulfonate: To a solution of [7-[4-fluoro-2-(2-methoxyethoxy)phenyl]-6-(4,5,6,7-tetrahydropyrazolo[1,5-a]pyrazin-2-yl)thieno[3,2-c]pyridin-4-yl]trifluoromethanesulfonate; 2,2,2-trifluoroacetic acid (28 mg, 0.041 mmol) and triethylamine (0.056 mL, 0.41 mmol) in DCM (3 mL) was added acryloyl chloride (0.0083 mL, 0.10 mmol) at −60° C. The mixture was stirred at −60° C. for 15 min. The mixture was poured into NaHCO₃ aqueous solution. The product was extracted with EtOAc, dried over anhydrous Na₂SO₄, concentrated and purified by preparative TLC (EtOAc/petroleum ether: 1/1) to give [7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]trifluoromethanesulfonate (22 mg, 86% yield). LCMS ESI (+) m/z 627.2 (M+H). ¹H NMR (400 MHz, CDCl₃) δ 7.58 (d, J=5.6 Hz, 1H), 7.49 (d, J=5.6 Hz, 1H), 7.21-7.25 (m, 1H), 6.75-6.61 (m, 2H), 6.55-6.62 (m, 1H), 6.15-6.39 (m, 2H), 5.80 (d, J=10.4 Hz, 1H), 4.78 (s, 2H), 3.90-4.15 (m, 5H), 3.80-3.87 (m, 1H), 3.34 (t, J=4.6 Hz, 2H), 3.03 (s, 3H).

Step G: Preparation of tert-butyl 6-[7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]-3,4-dihydro-1H-isoquinoline-2-carboxylate: To a solution of [7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]trifluoromethanesulfonate (11 mg, 0.018 mmol), sodium carbonate (6.1 mg, 0.058 mmol), and tert-butyl 6-(4,4,5,5-tetramethyl-1,3,2-dioxaborolan-2-yl)-3,4-dihydro-1H-isoquinoline-2-carboxylate (10 mg, 0.028 mmol) in 1,4-dioxane (2 mL) and water (0.2 mL) was added tetrakis(triphenylphosphine)palladium(0) (2.2 mg, 0.0019 mmol). The mixture was stirred at 90° C. for 2 h under Ar. The mixture was concentrated and purified by preparative TLC (EtOAc) to give tert-butyl 6-[7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]-3,4-dihydro-1H-isoquinoline-2-carboxylate (9.8 mg, 79% yield).

Step G: Preparation of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,7-dihydro-4H-pyrazolo[1,5-a]pyrazin-5-yl]prop-2-en-1-one: To a solution of tert-butyl 6-[7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]-3,4-dihydro-1H-isoquinoline-2-carboxylate (9.8 mg, 0.014 mmol) in DCM (2 mL) was added TFA (0.5 mL). The mixture was stirred at 25° C. for 1 h. The mixture was concentrated and purified by reverse phase preparative HPLC to give 1-[2-[7-[4-fluoro-2-(2-methoxyethoxy)phenyl]-4-(1,2,3,4-tetrahydroisoquinolin-6-yl)thieno[3,2-c]pyridin-6-yl]-6,7-dihydro-4H-pyrazolo[1,5-a]pyrazin-5-yl]prop-2-en-1-one (3.7 mg, 40% yield) as a bis trifluoroacetic acid salt. LCMS ESI (+) m/z 611.2 (M+H). ¹H NMR (400 MHz, CD₃OD) δ 7.87-7.92 (m, 2H), 7.82 (d, J=5.6 Hz, 1H), 7.67 (d, J=5.6 Hz, 1H), 7.47 (d, J=8.0 Hz, 1H), 7.31 (dd, J=8.0, 6.8 Hz, 1H), 6.99 (dd, J=11.0, 2.4 Hz, 1H), 6.75-6.94 (m, 2H), 6.25-6.32 (m, 1H), 5.78-5.95 (m, 2H), 4.50 (s, 2H), 4.18-4.26 (m, 2H), 4.02-4.15 (m, 3H), 3.90-3.97 (m, 1H), 3.60 (t, J=6.4 Hz, 2H), 3.41 (t, J=4.8 Hz, 2H), 3.25-3.29 (m, 2H), 3.07 (s, 3H).

### Synthetic Example 22: Synthesis of 6-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-3,4-dihydro-2H-isoquinolin-1-one (Compound 118)

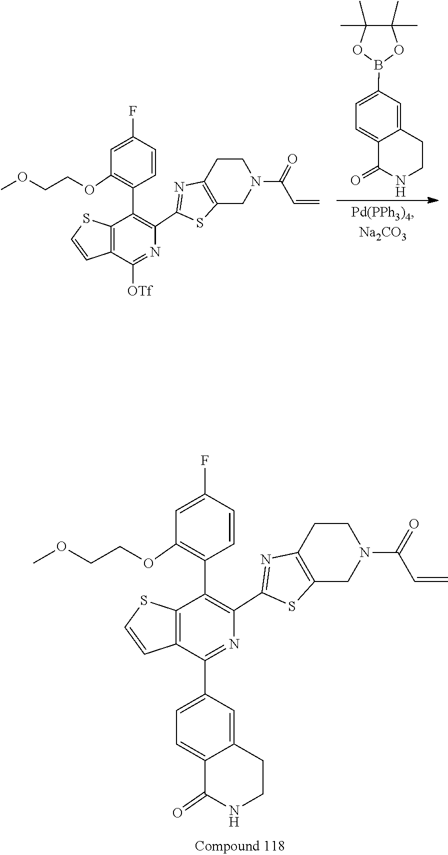

To a stirred solution of [7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]trifluoromethanesulfonate (30 mg, 0.047 mmol) in 1,4-dioxane (5 mL) were added 6-(4,4,5,5-tetramethyl-1,3,2-dioxaborolan-2-yl)-3,4-dihydro-2H-isoquinolin-1-one (25 mg, 0.093 mmol), Na₂CO₃ (9.9 mg, 0.093 mmol) and Pd(PPh₃)₄(5.4 mg, 0.0047 mmol). The mixture was stirred at 100° C. for 2 h under argon. The mixture was concentrated, and the residue was purified by reverse phase preparative HPLC to give 6-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-3,4-dihydro-2H-isoquinolin-1-one (20 mg, 67% yield) as a trifluoroacetic acid salt. LCMS ESI (+) m/z 641.2 (M+H). ¹H NMR (400 MHz, CD₃OD) δ 8.15 (d, J=8.0 Hz, 1H), 7.95 (d, J=8.0 Hz, 1H), 7.91 (s, 1H), 7.86 (d, J=5.5 Hz, 1H), 7.75 (d, J=5.5 Hz, 1H), 7.39 (dd, J=8.2, 6.8 Hz, 1H), 6.96 (d, J=9.4 Hz, 1H), 6.73-6.90 (m, 2H), 6.24 (d, J=16.2 Hz, 1H), 5.79 (d, J=10.5 Hz, 1H), 4.00-4.08 (m, 1H), 3.83-3.96 (m, 3H), 3.61 (t, J=6.6 Hz, 2H), 3.3-3.38 (m, 4H), 3.15 (t, J=6.5 Hz, 2H), 3.00 (s, 3H), 2.65-2.80 (m, 2H).

### Synthetic Example 23: Synthesis of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,8-dihydro-5H-imidazo[1,2-a]pyrazin-7-yl]prop-2-en-1-one (Compound 101)

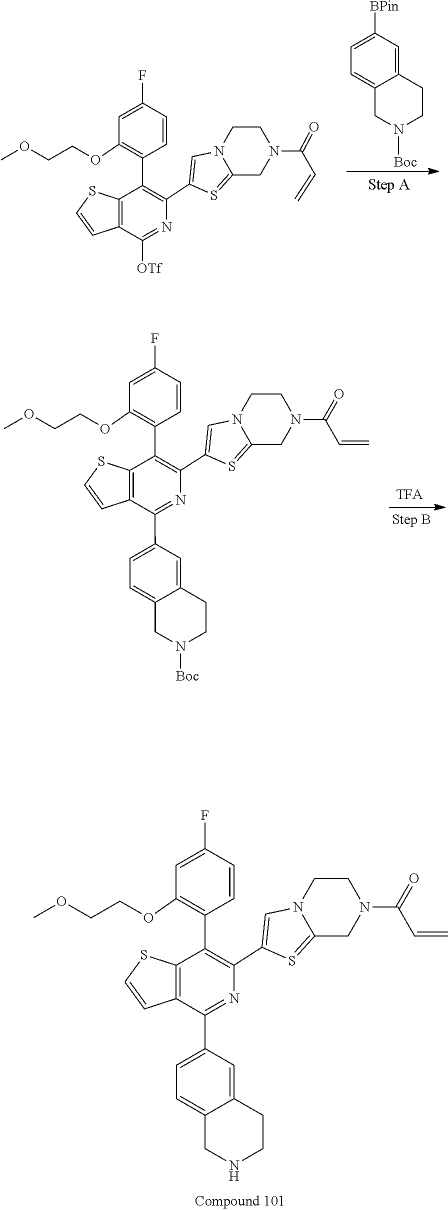

Step A: Preparation of tert-butyl 6-[7-[4-fluoro-2-(2-methoxyethoxy)phenyl]-6-(7-prop-2-enoyl-6,8-dihydro-5H-imidazo[1,2-a]pyrazin-2-yl)thieno[3,2-c]pyridin-4-yl]-3,4-dihydro-1H-isoquinoline-2-carboxylate: To a solution of [7-[4-fluoro-2-(2-methoxyethoxy)phenyl]-6-(7-prop-2-enoyl-6,8-dihydro-5H-imidazo[1,2-a]pyrazin-2-yl)thieno[3,2-c]pyridin-4-yl]trifluoromethanesulfonate (prepared as described in Synthetic Example 21 Steps C to F, substituting tert-butyl 2-cyano-6,8-dihydro-5H-imidazo[1,2-a]pyrazine-7-carboxylate for tert-butyl 2-cyano-6,7-dihydropyrazolo[1,5-a]pyrazine-5(4H)-carboxylate in Step C, 60 mg, 0.096 mmol), tert-butyl 6-(4,4,5,5-tetramethyl-1,3,2-dioxaborolan-2-yl)-3,4-dihydro-1H-isoquinoline-2-carboxylate (52 mg, 0.14 mmol) and Na₂CO₃ (20 mg, 0.19 mmol) in 1,4-dioxane (2 mL) and water (0.2 mL) was added Pd(PPh₃)₄(11 mg, 0.0096 mmol) under nitrogen. The mixture was stirred at 100° C. for 2 hours. The solvent was removed under reduced pressure, and the residue was purified by preparative TLC (MeOH/DCM=1/10) to afford tert-butyl 6-[7-[4-fluoro-2-(2-methoxyethoxy)phenyl]-6-(7-prop-2-enoyl-6,8-dihydro-5H-imidazo[1,2-a]pyrazin-2-yl)thieno[3,2-c]pyridin-4-yl]-3,4-dihydro-1H-isoquinoline-2-carboxylate (25 mg, 37% yield) as a yellow solid. LCMS ESI (+) m/z 710.4 (M+H).

Step B: Preparation of 1-[2-[7-[4-fluoro-2-(2-methoxyethoxy)phenyl]-4-(1,2,3,4-tetrahydroisoquinolin-6-yl)thieno[3,2-c]pyridin-6-yl]-6,8-dihydro-5H-imidazo[1,2-a]pyrazin-7-yl]prop-2-en-1-one: To a solution of tert-butyl 6-[7-[4-fluoro-2-(2-methoxyethoxy)phenyl]-6-(7-prop-2-enoyl-6,8-dihydro-5H-imidazo[1,2-a]pyrazin-2-yl)thieno[3,2-c]pyridin-4-yl]-3,4-dihydro-1H-isoquinoline-2-carboxylate (25 mg, 0.035 mmol) in DCM (2 mL) was added trifluoroacetic acid (0.70 mL). The mixture was stirred at 20° C. for 1 hour. The mixture was concentrated and purified by reverse phase preparative HPLC to get 1-[2-[7-[4-fluoro-2-(2-methoxyethoxy)phenyl]-4-(1,2,3,4-tetrahydroisoquinolin-6-yl)thieno[3,2-c]pyridin-6-yl]-6,8-dihydro-5H-imidazo[1,2-a]pyrazin-7-yl]prop-2-en-1-one (17 mg, 78% yield) as a bis trifluoroacetic acid salt. LCMS ESI (+) m/z 610.3 (M+H). ¹H NMR (400 MHz, CD₃OD) δ 7.91-7.97 (m, 2H), 7.87 (d, J=5.6 Hz, 1H), 7.74 (d, J=5.6 Hz, 1H), 7.49 (d, J=8.0 Hz, 1H), 7.39-7.45 (m, 1H), 7.14 (dd, J=11.0, 2.3 Hz, 1H), 6.97-7.05 (m, 1H), 6.73-6.91 (m, 1H), 6.52 (s, 1H), 6.29-6.38 (m, 1H), 5.88 (dd, J=10.6, 1.7 Hz, 1H), 5.02-5.21 (m, 2H), 4.52 (s, 2H), 4.04-4.22 (m, 6H), 3.62 (t, J=6.3 Hz, 2H), 3.38-3.45 (m, 2H), 3.03 (s, 3H).

### Synthetic Example 24: Synthesis of 1-[2-[4-[1-(azetidin-3-yl)pyrazol-4-yl]-7-[4-fluoro-2-(2-methoxyethoxy)phenyl]thieno[3,2-c]pyridin-6-yl]-6,7-dihydro-4H-thiazolo[5,4-c]pyridin-5-yl]prop-2-en-1-one (Compound 132)

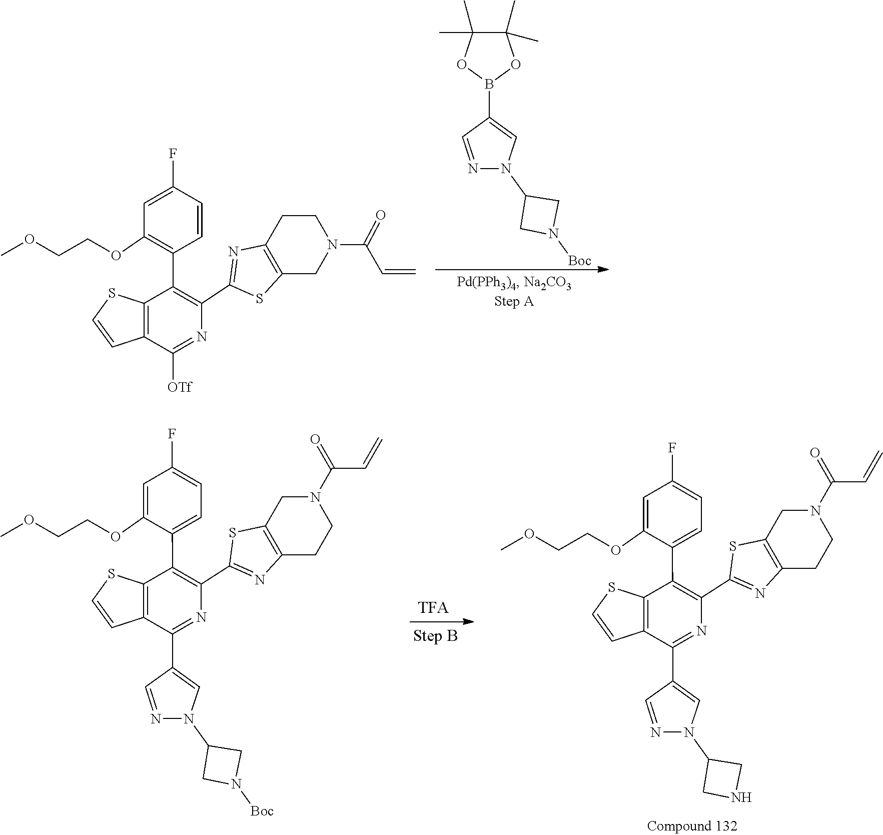 [SMILES: C=CC(=O)N1CCc2nc(-c3nc(OS(=O)(=O)C(F)(F)F)c4ccsc4c3-c3ccc(F)cc3OCCOC)sc2C1 ; CC(C)(C)OC(=O)N1CC(n2cc(B3OC(C)(C)C(C)(C)O3)cn2)C1 ; C=CC(=O)N1CCc2nc(-c3nc(-c4cnn(C5CN(C(=O)OC(C)(C)C)C5)c4)c4ccsc4c3-c3ccc(F)cc3OCCOC)sc2C1 ; C=CC(=O)N1CCc2nc(-c3nc(-c4cnn(C5CNC5)c4)c4ccsc4c3-c3ccc(F)cc3OCCOC)sc2C1]

Step A: Preparation of tert-butyl 3-[4-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]pyrazol-1-yl]azetidine-1-carboxylate: To a stirred solution of [7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]trifluoromethanemethanesulfonate (30 mg, 0.047 mmol) in 1,4-dioxane (5 mL) and water (0.5 mL) were added tert-butyl 3-[4-(4,4,5,5-tetramethyl-1,3,2-dioxaborolan-2-yl)pyrazol-1-yl]azetidine-1-carboxylate (33 mg, 0.0933 mmol), Na₂CO₃ (9.6 mg, 0.093 mmol) and Pd(PPh₃)₄(5.4 mg, 0.0047 mmol). The mixture was stirred at 100° C. for 2 h under argon. The mixture was concentrated. The residue was purified by preparative TLC (EtOAc/petroleum ether: 1/2) to give tert-butyl 3-[4-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]pyrazol-1-yl]azetidine-1-carboxylate (40 mg, 90% yield) as a white solid. LCMS ESI (+) m/z 717.4 (M+H).

Step B: Preparation of 1-[2-[4-[1-(azetidin-3-yl)pyrazol-4-yl]-7-[4-fluoro-2-(2-methoxyethoxy)phenyl]thieno[3,2-c]pyridin-6-yl]-6,7-dihydro-4H-thiazolo[5,4-c]pyridin-5-yl]prop-2-en-1-one: To a stirred solution of tert-butyl 3-[4-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]pyrazol-1-yl]azetidine-1-carboxylate (40 mg, 0.056 mmol) in DCM (3 mL) was added trifluoroacetic acid (1.0 mL). The solution was stirred at 18° C. for 1 h. The mixture was concentrated and purified by reverse phase preparative HPLC to give 1-[2-[4-[1-(azetidin-3-yl)pyrazol-4-yl]-7-[4-fluoro-2-(2-methoxyethoxy)phenyl]thieno[3,2-c]pyridin-6-yl]-6,7-dihydro-4H-thiazolo[5,4-c]pyridin-5-yl]prop-2-en-1-one (24 mg, 92% yield) as a trifluoroacetic acid salt. LCMS ESI (+) m/z 617.2 (M+H). ¹H NMR (400 MHz, CD₃OD) δ 8.56 (s, 1H), 8.49 (s, 1H), 7.92 (d, J=5.6 Hz, 1H), 7.85 (d, J=5.6 Hz, 1H), 7.35 (dd, J=8.3, 6.7 Hz, 1H), 6.77-6.96 (m, 3H), 6.17-6.29 (m, 1H), 5.75-5.83 (m, 1H), 5.55-5.64 (m, 1H), 4.87-4.92 (m, 2H), 4.59-4.70 (m, 4H), 3.98-4.06 (m, 1H), 3.82-3.95 (m, 3H), 3.23-3.30 (m, 2H), 2.98 (s, 3H), 2.68-2.78 (m, 2H).

### Synthetic Example 25: Synthesis of 1-(2-(7-(4-fluoro-2-(2-methoxyethoxy)phenyl)-4-(1-isopropylpiperidin-4-yl)thieno[3,2-c]pyridin-6-yl)-6,7-dihydrothiazolo[5,4-c]pyridin-5(4H)-yl)prop-2-en-1-one (Compound 143)

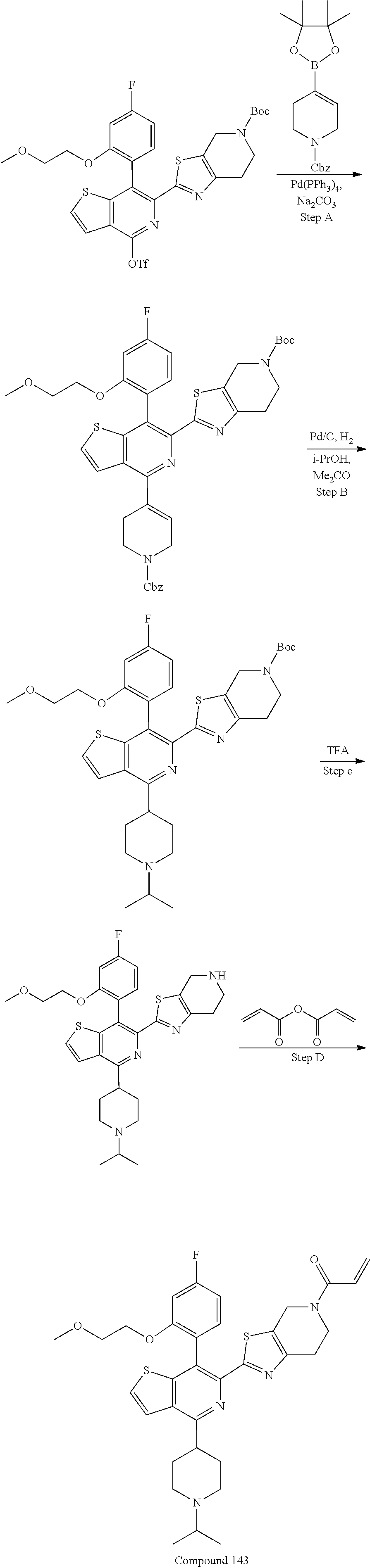

Step A: Preparation of tert-butyl 2-(4-(1-((benzyloxy)carbonyl)-1,2,3,6-tetrahydropyridin-4-yl)-7-(4-fluoro-2-(2-methoxyethoxy)phenyl)thieno[3,2-c]pyridin-6-yl)-6,7-dihydrothiazolo[5,4-c]pyridine-5(4H)-carboxylate: To a solution of tert-butyl 2-(7-(4-fluoro-2-(2-methoxyethoxy)phenyl)-4-(((trifluoromethyl)sulfonyl)oxy)thieno[3,2-c]pyridin-6-yl)-6,7-dihydrothiazolo[5,4-c]pyridine-5(4H)-carboxylate (80 mg, 0.12 mmol) and benzyl 4-(4,4,5,5-tetramethyl-1,3,2-dioxaborolan-2-yl)-5,6-dihydropyridine-1(2H)-carboxylate (110 mg, 0.139 mmol) in 1,4-dioxane (5 mL) and water (0.5 mL) were added Pd(PPh₃)₄(14 mg, 0.012 mmol) and Na₂CO₃ (25 mg, 0.23 mmol). The solution was stirred at 90° C. for 2 h under N₂. The solution was diluted with EtOAc and washed with brine. The organic phase was dried over Na₂SO₄, concentrated and purified by preparative TLC (EtOAc/PE: 1/3) to give tert-butyl 2-(4-(1-((benzyloxy)carbonyl)-1,2,3,6-tetrahydropyridin-4-yl)-7-(4-fluoro-2-(2-methoxyethoxy)phenyl)thieno[3,2-c]pyridin-6-yl)-6,7-dihydrothiazolo[5,4-c]pyridine-5(4H)-carboxylate (85 mg, 97% yield) as a yellow solid. LCMS ESI (+) m/z 757.4 (M+H).

Step B: Preparation of tert-butyl 2-(7-(4-fluoro-2-(2-methoxyethoxy)phenyl)-4-(1-isopropylpiperidin-4-yl)thieno[3,2-c]pyridin-6-yl)-6,7-dihydrothiazolo[5,4-c]pyridine-5(4H)-carboxylate: To a solution of tert-butyl 2-(4-(1-((benzyloxy)carbonyl)-1,2,3,6-tetrahydropyridin-4-yl)-7-(4-fluoro-2-(2-methoxyethoxy)phenyl)thieno[3,2-c]pyridin-6-yl)-6,7-dihydrothiazolo[5,4-c]pyridine-5(4H)-carboxylate (53 mg, 0,070 mmol) in i-PrOH (5 mL) and acetone (1 mL) was added Pd/C (53 mg). The mixture was stirred for 48 h at 40° C. under H₂ atmosphere. The solution was filtered through Celite® and washed with DCM. The filtrate was concentrated in vacuo and purified by preparative TLC (EtOAc/petroleum ether 1:4) to give tert-butyl 2-(7-(4-fluoro-2-(2-methoxyethoxy)phenyl)-4-(1-isopropylpiperidin-4-yl)thieno[3,2-c]pyridin-6-yl)-6,7-dihydrothiazolo[5,4-c]pyridine-5(4H)-carboxylate (26 mg, 56% yield) as a yellow solid. LCMS ESI (+) m/z 667.3 (M+H).

Step C: Preparation of 2-(7-(4-fluoro-2-(2-methoxyethoxy)phenyl)-4-(1-isopropylpiperidin-4-yl)thieno[3,2-c]pyridin-6-yl)-4,5,6,7-tetrahydrothiazolo[5,4-c]pyridine: To a solution of tert-butyl 2-(7-(4-fluoro-2-(2-methoxyethoxy)phenyl)-4-(1-isopropylpiperidin-4-yl)thieno[3,2-c]pyridin-6-yl)-6,7-dihydrothiazolo[5,4-c]pyridine-5(4H)-carboxylate (26 mg, 0.039 mmol) in DCM (2 mL) was added trifluoroacetic acid (2.0 mL). The solution was stirred at rt for 1 h at 0° C. The solution was concentrated. The crude product was purified by recrystallization (methyl tert-butyl ether) to give 2-(7-(4-fluoro-2-(2-methoxyethoxy)phenyl)-4-(1-isopropylpiperidin-4-yl)thieno[3,2-c]pyridin-6-yl)-4,5,6,7-tetrahydrothiazolo[5,4-c]pyridine (22 mg, 85% yield) as a yellow solid. LCMS ESI (+) m/z 567.4 (M+H).

Step D: Preparation of 1-(2-(7-(4-fluoro-2-(2-methoxyethoxy)phenyl)-4-(1-isopropylpiperidin-4-yl)thieno[3,2-c]pyridin-6-yl)-6,7-dihydrothiazolo[5,4-c]pyridin-5(4H)-yl)prop-2-en-1-one: To a solution of 2-(7-(4-fluoro-2-(2-methoxyethoxy)phenyl)-4-(1-isopropylpiperidin-4-yl)thieno[3,2-c]pyridin-6-yl)-4,5,6,7-tetrahydrothiazolo[5,4-c]pyridine (22 mg, 0.033 mmol) and DIEA (13 mg, 0.10 mmol) in DCM (1 mL) was added acrylic anhydride (4.4 mg, 0.35 mmol). The solution was stirred for 1 h at 0° C. under N₂. The solution was diluted with DCM and washed with NaHCO₃ aqueous solution twice. The organic phase was dried over Na₂SO₄, concentrated and purified by reverse phase preparative HPLC to give 1-(2-(7-(4-fluoro-2-(2-methoxyethoxy)phenyl)-4-(1-isopropylpiperidin-4-yl)thieno[3,2-c]pyridin-6-yl)-6,7-dihydrothiazolo[5,4-c]pyridin-5(4H)-yl)prop-2-en-1-one (4.1 mg, 20% yield) as a bis trifluoroacetic acid salt. LCMS ESI (+) m/z 621.3 (M+H). ¹H NMR (400 MHz, CD₃OD) δ 7.77-7.82 (m, 2H), 7.29 (t, J=5.9 Hz, 1H), 6.80-6.91 (m, 3H), 6.25 (d, J=16 Hz, 1H), 5.80 (d, J=10.4 Hz, 1H), 3.99-4.03 (m, 1H), 3.88-3.91 (m, 2H), 3.82-3.85 (m, 2H), 3.61-3.79 (m, 3H), 3.35-3.39 (m, 2H), 3.25-3.35 (m, 2H), 3.06 (s, 3H), 2.63-2.71 (m, 2H), 2.63-2.71 (m, 2H), 2.31-2.49 (m, 4H), 1.45 (d, J=6.4 Hz, 1H).

### Synthetic Example 26: Synthesis of 4-[[5-fluoro-2-[4-(1-methylpyrazol-4-yl)-6-(5-prop-2-enoyl-6,7-dihydro-4H-thiazolo[5,4-c]pyridin-2-yl)thieno[3,2-c]pyridin-7-yl]phenoxy]methyl]piperidin-2-one (Compound 144)

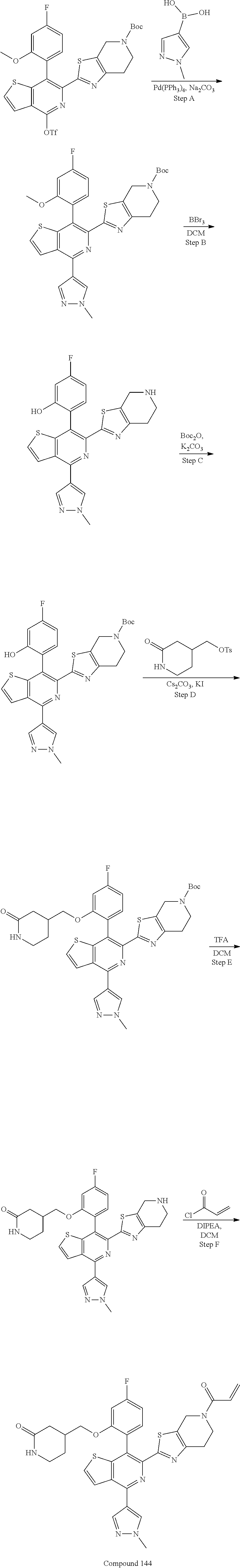

Step A: Preparation of tert-butyl 2-[7-(4-fluoro-2-methoxy-phenyl)-4-(1-methylpyrazol-4-yl)thieno[3,2-c]pyridin-6-yl]-6,7-dihydro-4H-thiazolo[5,4-c]pyridine-5-carboxylate: A mixture of tert-butyl 2-[7-(4-fluoro-2-methoxy-phenyl)-4-(trifluoromethylsulfonyloxy)thieno[3,2-c]pyridin-6-yl]-6,7-dihydro-4H-thiazolo[5,4-c]pyridine-5-carboxylate (143 mg, 0.221 mmol), (1-methylpyrazol-4-yl)boronic acid (56 mg, 0.44 mmol), tetrakis(triphenylphosphine)palladium(0) (26 mg, 0.022 mmol) and sodium carbonate (47 mg, 0.44 mmol) in 1,4-dioxane (2 mL)/water (0.2 mL) was stirred at 90° C. under N₂ for 2 hours. The mixture was concentrated and purified by preparative TLC (EtOAc) to give tert-butyl 2-[7-(4-fluoro-2-methoxy-phenyl)-4-(1-methylpyrazol-4-yl)thieno[3,2-c]pyridin-6-yl]-6,7-dihydro-4H-thiazolo[5,4-c]pyridine-5-carboxylate (100 mg, 78% yield). LCMS ESI (+) m/z 578.2 (M+H).

Step B: Preparation of 5-fluoro-2-(4-(1-methyl-1H-pyrazol-4-yl)-6-(4,5,6,7-tetrahydrothiazolo[5,4-c]pyridin-2-yl)thieno[3,2-c]pyridin-7-yl)phenol: To a solution of tert-butyl 2-[7-(4-fluoro-2-methoxy-phenyl)-4-(1-methylpyrazol-4-yl)thieno[3,2-c]pyridin-6-yl]-6,7-dihydro-4H-thiazolo[5,4-c]pyridine-5-carboxylate (90 mg, 0.16 mmol) in dry DCM (2 mL) was added boron tribromide (0.044 mL, 0.47 mmol) at −20° C. The mixture was stirred at 25° C. for 12 hours. The mixture was cooling to −20° C. MeOH was added dropwise to quench the reaction. NaHCO₃ solution was added to adjust the pH to 6-7. The product was extracted with EtOAc, dried over Na₂SO₄ and concentrated to afford crude 5-fluoro-2-(4-(1-methyl-1H-pyrazol-4-yl)-6-(4,5,6,7-tetrahydrothiazolo[5,4-c]pyridin-2-yl)thieno[3,2-c]pyridin-7-yl)phenol (90 mg) which was directly used in the next step without further purification. LCMS ESI (+) m/z 464.1 (M+H).

Step C: Preparation of tert-butyl 2-[7-(4-fluoro-2-hydroxy-phenyl)-4-(1-methylpyrazol-4-yl)thieno[3,2-c]pyridin-6-yl]-6,7-dihydro-4H-thiazolo[5,4-c]pyridine-5-carboxylate: To a solution of 5-fluoro-2-[4-(1-methylpyrazol-4-yl)-6-(4,5,6,7-tetrahydrothiazolo[5,4-c]pyridin-2-yl)thieno[3,2-c]pyridin-7-yl]phenol (90 mg, 0.19 mmol) and potassium carbonate (54 mg, 0.39 mmol) in THF (2 mL) was added di-tert-butyl dicarbonate (0.045 mL, 0.19 mmol). The mixture was stirred at 25° C. for 2 hours. The mixture was diluted with water. 1 N aqueous HCl was added to adjust the pH to 5. The product was extracted with EtOAc, dried over Na₂SO₄, concentrated and purified by preparative TLC (petroleum ether:EtOAc=1:2) to give tert-butyl 2-[7-(4-fluoro-2-hydroxy-phenyl)-4-(1-methylpyrazol-4-yl)thieno[3,2-c]pyridin-6-yl]-6,7-dihydro-4H-thiazolo[5,4-c]pyridine-5-carboxylate (71 mg, 65% yield). LCMS ESI (+) m/z 564.2 (M+H).

Step D: Preparation of tert-butyl 2-[7-[4-fluoro-2-[(2-oxo-4-piperidyl)methoxy]phenyl]-4-(1-methylpyrazol-4-yl)thieno[3,2-c]pyridin-6-yl]-6,7-dihydro-4H-thiazolo[5,4-c]pyridine-5-carboxylate: A mixture of tert-butyl 2-[7-(4-fluoro-2-hydroxy-phenyl)-4-(1-methylpyrazol-4-yl)thieno[3,2-c]pyridin-6-yl]-6,7-dihydro-4H-thiazolo[5,4-c]pyridine-5-carboxylate (80 mg, 0.14 mmol), (2-oxo-4-piperidyl)methyl 4-methylbenzenesulfonate (80 mg, 0.28 mmol), cesium carbonate (139 mg, 0.426 mmol) and KI (4.6 mg, 0.028 mmol) in acetonitrile (4 mL) was stirred at 80° C. for 12 hours. The mixture was diluted with EtOAc, washed by brine, dried over Na₂SO₄ and concentrated. The residue was purified by silica gel column (DCM:MeOH=20:1 to 10:1) to give tert-butyl 2-[7-[4-fluoro-2-[(2-oxo-4-piperidyl)methoxy]phenyl]-4-(1-methylpyrazol-4-yl)thieno[3,2-c]pyridin-6-yl]-6,7-dihydro-4H-thiazolo[5,4-c]pyridine-5-carboxylate (90 mg, 94% yield). LCMS ESI (+) m/z 675.3 (M+H).

Step E: Preparation of 4-[[5-fluoro-2-[4-(1-methylpyrazol-4-yl)-6-(5-prop-2-enoyl-6,7-dihydro-4H-thiazolo[5,4-c]pyridin-2-yl)thieno[3,2-c]pyridin-7-yl]phenoxy]methyl]piperidin-2-one: To a solution of 4-[[5-fluoro-2-[4-(1-methylpyrazol-4-yl)-6-(4,5,6,7-tetrahydrothiazolo[5,4-c]pyridin-2-yl)thieno[3,2-c]pyridin-7-yl]phenoxy]methyl]piperidin-2-one (70 mg, 0.12 mmol) and DIPEA (0.064 mL, 0.37 mmol) in DCM (3 mL) was added acryloyl chloride (0.011 mL, 0.13 mmol) at 0° C. The mixture was stirred at 0° C. for 40 min. NaHCO₃ solution was added to quench the reaction. The product was extracted with DCM, dried over Na₂SO₄ and concentrated. The residue was purified by reverse phase preparative HPLC to give 4-[[5-fluoro-2-[4-(1-methylpyrazol-4-yl)-6-(5-prop-2-enoyl-6,7-dihydro-4H-thiazolo[5,4-c]pyridin-2-yl)thieno[3,2-c]pyridin-7-yl]phenoxy]methyl]piperidin-2-one (26 mg, 34% yield) as a bis trifluoroacetic acid salt. LCMS ESI (+) m/z 629.3 (M+H). ¹H NMR (400 MHz, CD₃OD) δ 8.53 (d, J=6.2 Hz, 1H), 8.31 (d, J=4.2 Hz, 1H), 7.94-7.99 (m, 2H), 7.39 (t, J=8.8 Hz, 1H), 6.89-7.03 (m, 3H), 6.25 (d, J=16.7 Hz, 1H), 5.80 (d, J=10.0 Hz, 1H), 4.07 (s, 3H), 3.79-3.93 (m, 4H), 2.99-3.01 (m, 2H), 2.82-2.86 (m, 2H), 1.97-2.03 (m, 2H), 1.47-1.73 (m, 2H), 1.08-1.20 (m, 1H).

### Synthetic Example 27: Synthesis of 1-[2-[7-[4-fluoro-2-(2-methoxyethoxy)phenyl]-4-(2-imino-2-oxo-1,3-dihydro-2-benzothiophen-5-yl)thieno[3,2-c]pyridin-6-yl]-6,7-dihydro-4H-thiazolo[5,4-c]pyridin-5-yl]prop-2-en-1-one (Compound 151)

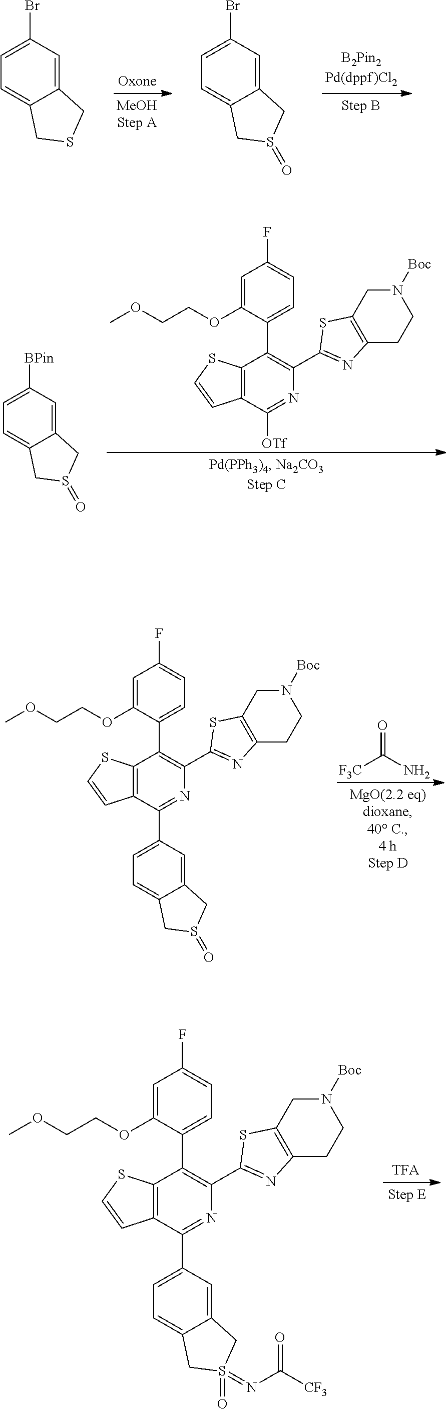

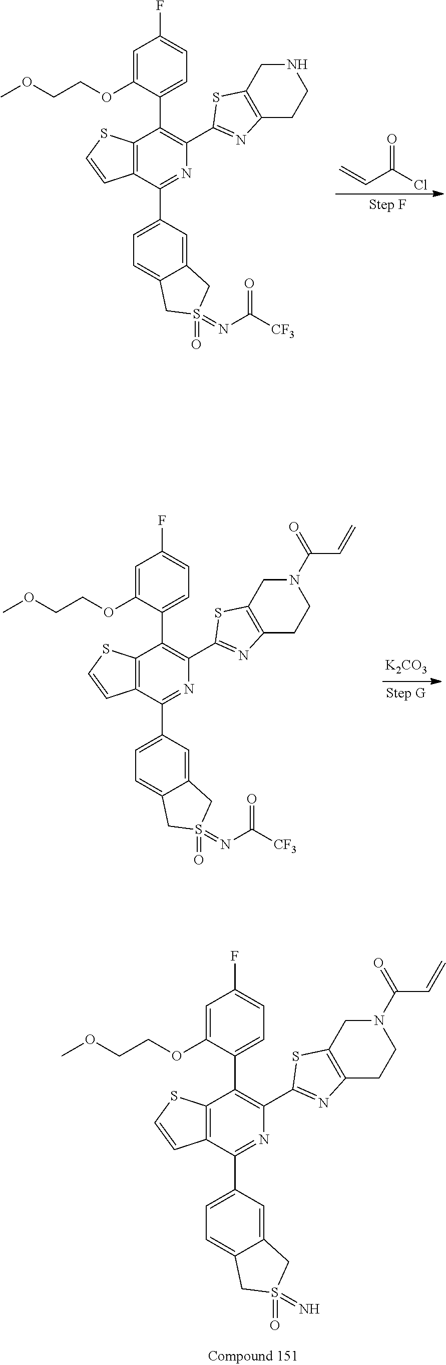

Step A: Preparation of 5-bromo-1,3-dihydro-2-benzothiophene-2-oxide: To a solution of 5-bromo-1,3-dihydro-2-benzothiophene (500 mg, 2.32 mmol) in MeOH (4 mL) was added Oxone (2.14 g, 3.49 mmol) under Ar. The mixture was stirred at 0° C. for 1.5 h. The reaction was poured into water and the product was extracted with EtOAc. The organic layers were washed with brine, dried over anhydrous Na₂SO₄, concentrated and purified by column chromatography (EtOAc/petroleum ether: 1/5) to give 5-bromo-1,3-dihydro-2-benzothiophene-2-oxide (117 mg, 22% yield), LCMS ESI (+) m/z 231.0 (M+H).

Step B: Preparation of 5-(4,4,5,5-tetramethyl-1,3,2-dioxaborolan-2-yl)-1,3-dihydro-2-benzothiophene 2-oxide: To a solution of 5-bromo-1,3-dihydro-2-benzothiophene 2-oxide (96 mg, 0.42 mmol), bis(pinacolato)diboron (158 mg, 0.623 mmol), potassium acetate (122 mg, 1.25 mmol) in 1,4-dioxane (2 mL) was added [1,1′-bis(diphenylphosphino)ferrocene]dichloropalladium(II) (30 mg, 0.042 mmol). The mixture was stirred at 90° C. for 6 h under Ar. The mixture was poured into water. The product was extracted with EtOAc, dried over anhydrous Na₂SO₄, concentrated and purified by preparative TLC (DCM/MeOH 40/1) to give 5-(4,4,5,5-tetramethyl-1,3,2-dioxaborolan-2-yl)-1,3-dihydro-2-benzothiophene 2-oxide (107 mg, 93% yield). LCMS ESI (+) m/z 279.1 (M+H).

Step C: Preparation of tert-butyl 2-[7-[4-fluoro-2-(2-methoxyethoxy)phenyl]-4-(2-oxo-1,3-dihydro-2-benzothiophen-5-yl)thieno[3,2-c]pyridin-6-yl]-6,7-dihydro-4H-thiazolo[5,4-c]pyridine-5-carboxylate: To a solution of tert-butyl 2-[7-[4-fluoro-2-(2-methoxyethoxy)phenyl]-4-(trifluoromethylsulfonyloxy)thieno[3,2-c]pyridin-6-yl]-6,7-dihydro-4H-thiazolo[5,4-c]pyridine-5-carboxylate (62 mg, 0.090 mmol), 5-(4,4,5,5-tetramethyl-1,3,2-dioxaborolan-2-yl)-1,3-dihydro-2-benzothiophene-2-oxide (25 mg, 0.090 mmol) and sodium carbonate (9.0 mg, 0.18 mmol) in 1,4-dioxane (10 mL) and water (1 mL) was added tetrakis(triphenylphosphine)palladium(0) (10 mg, 0.0090 mmol). The mixture was stirred at 100° C. for 2 h under Ar. The mixture was poured into water and the product was extracted with EtOAc. The organic layers were dried over anhydrous Na₂SO₄, concentrated and purified by preparative TLC (DCM/MeOH 20/1) to give tert-butyl 2-[7-[4-fluoro-2-(2-methoxyethoxy)phenyl]-4-(2-oxo-1,3-dihydro-2-benzothiophen-5-yl)thieno[3,2-c]pyridin-6-yl]-6,7-dihydro-4H-thiazolo[5,4-c]pyridine-5-carboxylate (45 mg, 72% yield). LCMS ESI (+) m/z 692.2 (M+H).

Step D: Preparation of tert-butyl 2-[7-[4-fluoro-2-(2-methoxyethoxy)phenyl]-4-[2-oxo-2-(2,2,2-trifluoroacetyl)imino-1,3-dihydro-2-benzothiophen-5-yl]thieno[3,2-c]pyridin-6-yl]-6,7-dihydro-4H-thiazolo[5,4-c]pyridine-5-carboxylate: To a solution of tert-butyl 2-[7-[4-fluoro-2-(2-methoxyethoxy)phenyl]-4-(2-oxo-1,3-dihydro-2-benzothiophen-5-yl)thieno[3,2-c]pyridin-6-yl]-6,7-dihydro-4H-thiazolo[5,4-c]pyridine-5-carboxylate (40 mg, 0.058 mmol), 2,2,2-trifluoroacetamide (14 mg, 0.13 mmol) and magnesium oxide (10 mg, 0.25 mmol) in 1,4-dioxane (1.5 mL) was added rhodium acetate (1.6 mg, 0.0058 mmol). The mixture was stirred at 40° C. for 3 h under Ar. The mixture was poured into water. The product was extracted with EtOAc, dried over anhydrous Na₂SO₄, concentrated and purified by preparative TLC (DCM/MeOH: 20/1) to give tert-butyl 2-[7-[4-fluoro-2-(2-methoxyethoxy)phenyl]-4-[2-oxo-2-(2,2,2-trifluoroacetyl)imino-1,3-dihydro-2-benzothiophen-5-yl]thieno[3,2-c]pyridin-6-yl]-6,7-dihydro-4H-thiazolo[5,4-c]pyridine-5-carboxylate (35 mg, 75% yield).

Step E: Preparation of 2,2,2-trifluoro-N-[5-[7-[4-fluoro-2-(2-methoxyethoxy)phenyl]-6-(4,5,6,7-tetrahydrothiazolo[5,4-c]pyridin-2-yl)thieno[3,2-c]pyridin-4-yl]-2-oxo-1,3-dihydro-2-benzothiophen-2-ylidene]acetamide: To a solution of tert-butyl 2-[7-[4-fluoro-2-(2-methoxyethoxy)phenyl]-4-[2-oxo-2-(2,2,2-trifluoroacetyl)imino-1,3-dihydro-2-benzothiophen-5-yl]thieno[3,2-c]pyridin-6-yl]-6,7-dihydro-4H-thiazolo[5,4-c]pyridine-5-carboxylate (35 mg, 0.044 mmol) in DCM (3 mL) was added trifluoroacetic acid (0.50 mL). The mixture was stirred at 25° C. for 1 h. The mixture was concentrated and diluted with EtOAc. The organic layers were washed with NaHCO₃ aqueous solution, dried over Na₂SO₄, filtered and concentrated to dryness to give 2,2,2-trifluoro-N-[5-[7-[4-fluoro-2-(2-methoxyethoxy)phenyl]-6-(4,5,6,7-tetrahydrothiazolo[5,4-c]pyridin-2-yl)thieno[3,2-c]pyridin-4-yl]-2-oxo-1,3-dihydro-2-benzothiophen-2-ylidene]acetamide (30 mg, 98% yield). LCMS ESI (+) m/z 703.1 (M+H).

Step F: Preparation of 2,2,2-trifluoro-N-[5-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-2-oxo-1,3-dihydro-2-benzothiophen-2-ylidene]acetamide: To a solution of 2,2,2-trifluoro-N-[5-[7-[4-fluoro-2-(2-methoxyethoxy)phenyl]-6-(4,5,6,7-tetrahydrothiazolo[5,4-c]pyridin-2-yl)thieno[3,2-c]pyridin-4-yl]-2-oxo-1,3-dihydro-2-benzothiophen-2-ylidene]acetamide (30 mg, 0.043 mmol) in DCM (3 mL) was added triethylamine (13 mg, 0.13 mmol) and acryloyl chloride (0.0038 mL, 0.047 mmol) at −60° C. The mixture was stirred at −60° C. for 0.5 h. The mixture was poured into NaHCO₃ aqueous solution. The product was extracted with EtOAc. The organics were washed with brine, dried over anhydrous Na₂SO₄, concentrated and purified by preparative TLC (DCM/MeOH 20/1) to give 2,2,2-trifluoro-N-[5-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-2-oxo-1,3-dihydro-2-benzothiophen-2-ylidene]acetamide (24 mg, 74% yield). LCMS ESI (+) m/z 757.2 (M+H).

Step G: Preparation of 1-[2-[7-[4-fluoro-2-(2-methoxyethoxy)phenyl]-4-(2-imino-2-oxo-1,3-dihydro-2-benzothiophen-5-yl)thieno[3,2-c]pyridin-6-yl]-6,7-dihydro-4H-thiazolo[5,4-c]pyridin-5-yl]prop-2-en-1-one: To a solution of 2,2,2-trifluoro-N-[5-[7-[4-fluoro-2-(2-methoxyethoxy)phenyl]-6-(5-prop-2-enoyl-6,7-dihydro-4H-thiazolo[5,4-c]pyridin-2-yl)thieno[3,2-c]pyridin-4-yl]-2-oxo-1,3-dihydro-2-benzothiophen-2-ylidene]acetamide (12 mg, 0.016 mmol) in methanol (1 mL) was added potassium carbonate (4.4 mg, 0.032 mmol). The mixture was stirred at 25° C. for 1 h. The mixture was poured into water. The product was extracted with EtOAc, dried over anhydrous Na₂SO₄, concentrated and purified by reverse phase preparative HPLC to give 1-[2-[7-[4-fluoro-2-(2-methoxyethoxy)phenyl]-4-(2-imino-2-oxo-1,3-dihydro-2-benzothiophen-5-yl)thieno[3,2-c]pyridin-6-yl]-6,7-dihydro-4H-thiazolo[5,4-c]pyridin-5-yl]prop-2-en-1-one (2.2 mg, 21% yield) as a trifluoroacetic acid salt. LCMS ESI (+) m/z 661.2 (M+H). ¹H NMR (400 MHz, CD₃OD) δ 8.02-8.09 (m, 2H), 7.84 (d, J=4.8 Hz, 1H), 7.74 (d, J=5.6 Hz, 1H), 7.66 (d, J=8.0 Hz, 1H), 7.37 (dd, J=8.4, 6.4 Hz, 1H), 6.94 (d, J=11.2 Hz, 1H), 6.82-6.88 (m, 2H), 6.24 (d, J=16.4 Hz, 1H), 5.80 (d, J=10.8 Hz, 1H), 5.05 (s, 2H), 5.02 (s, 2H), 4.00-4.06 (m, 1H), 3.82-3.95 (m, 3H), 3.00 (s, 3H), 2.65-2.72 (m, 2H).

### Synthetic Example 28: Synthesis of 1-[3-[4-[7-(4-fluoro-2-methoxy-phenyl)-4-(1,2,3,4-tetrahydroisoquinolin-6-yl)pyrazolo[1,5-a]pyrazin-6-yl]pyrazol-1-yl]azetidin-1-yl]prop-2-en-1-one (Compound 169)

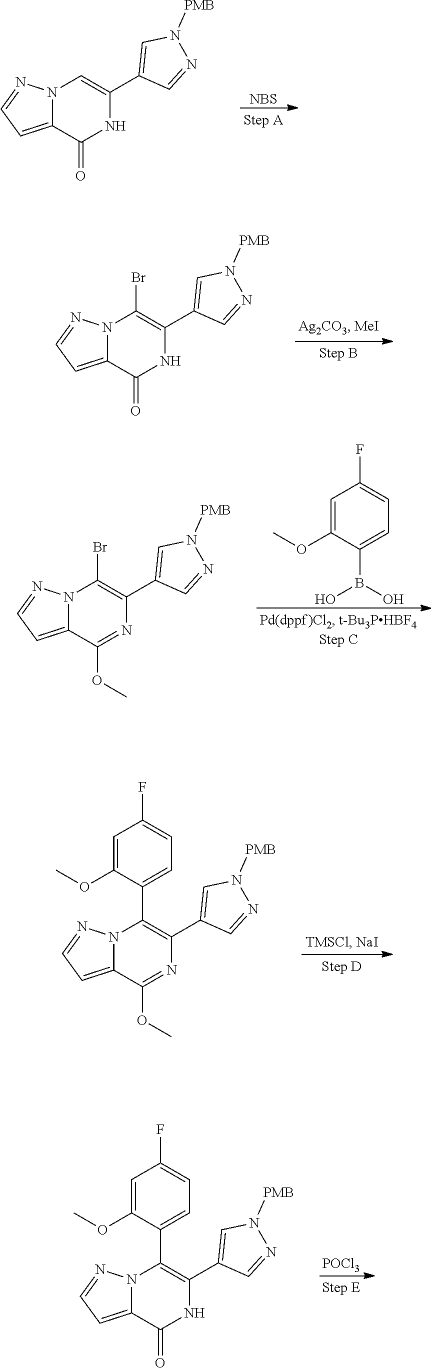

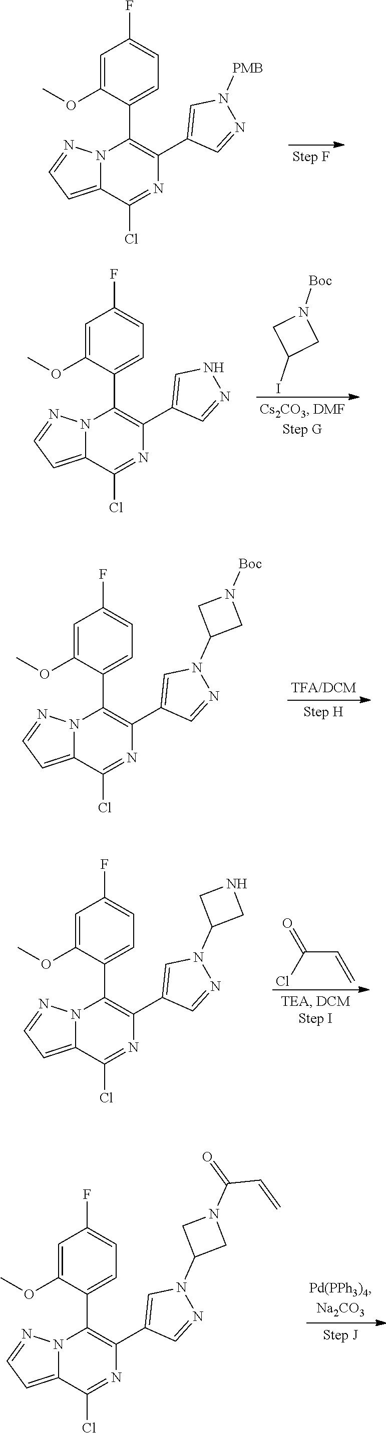

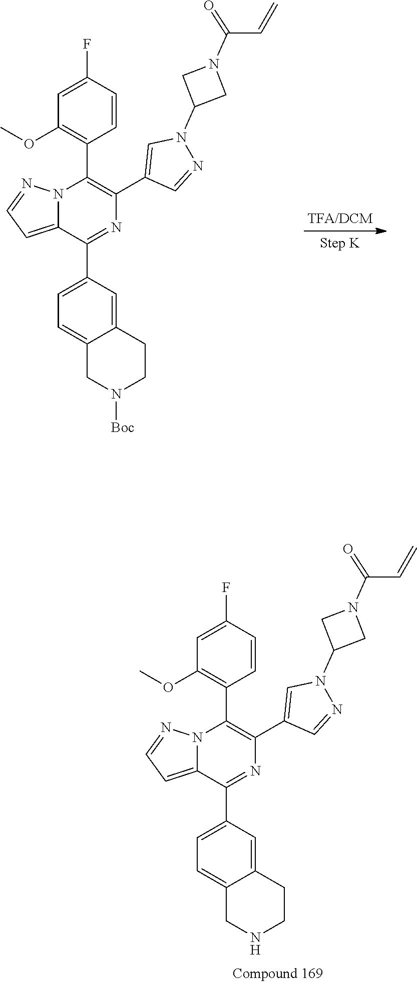

Step A: Preparation of 7-bromo-6-[1-[(4-methoxyphenyl)methyl]pyrazol-4-yl]-5H-pyrazolo[1,5-a]pyrazin-4-one: To a suspension of 6-[1-[(4-methoxyphenyl)methyl]pyrazol-4-yl]-5H-pyrazolo[1,5-a]pyrazin-4-one (400 mg, 1.24 mmol) in DMF (10 mL) was added N-bromosuccinimide (200 mg, 1.12 mmol) at 0° C. The mixture was stirred at 0° C. for 1 h. NaHSO₃ aqueous solution (5 mL) was added to quench the reaction. The mixture was diluted with water (15 mL). The solid was filtered, washed with water and MTBE, and dried in vacuo to give crude 7-bromo-6-[1-[(4-methoxyphenyl)methyl]pyrazol-4-yl]-5H-pyrazolo[1,5-a]pyrazin-4-one (248 mg, 50% yield) which was used in the next step without further purification. LCMS ESI (+) m/z 400, 402 (M+H).

Step B: Preparation of 7-bromo-4-methoxy-6-[1-[(4-methoxyphenyl)methyl]pyrazol-4-yl]pyrazolo[1,5-a]pyrazine: To a suspension of 7-bromo-6-[1-[(4-methoxyphenyl)methyl]pyrazol-4-yl]-5H-pyrazolo[1,5-a]pyrazin-4-one (248 mg, 0.62 mmol) in toluene (5 mL) was added methyl iodide (0.057 mL, 0.91 mmol), followed by silver carbonate (174 mg, 0.630 mmol). The mixture was stirred at 90° C. in a sealed tube for 16 h. The mixture was diluted with EtOAc and filtered. The filtrate was concentrated and purified by preparative TLC (EtOAc/petroleum ether: 1/2) to give 7-bromo-4-methoxy-6-[1-[(4-methoxyphenyl)methyl]pyrazol-4-yl]pyrazolo[1,5-a]pyrazine (100 mg, 39% yield).

Step C: Preparation of 7-(4-fluoro-2-methoxy-phenyl)-4-methoxy-6-[1-[(4-methoxyphenyl)methyl]pyrazol-4-yl]pyrazolo[1,5-a]pyrazine: To a solution of tri-tert-butylphosphonium tetrafluoroborate (14 mg, 0.049 mmol), cesium carbonate (207 mg, 0.636 mmol) and (4-fluoro-2-methoxy-phenyl)boronic acid (71 mg, 0.42 mmol) in 1,4-dioxane (4 mL) and water (0.4 mL) were added 7-bromo-4-methoxy-6-[1-[(4-methoxyphenyl)methyl]pyrazol-4-yl]pyrazolo[1,5-a]pyrazine (100 mg, 0.241 mmol) and 1,1′-bis(diphenylphosphino)ferrocenedichloro palladium(II) dichloromethane complex (21 mg, 0.029 mmol). The mixture was stirred at 100° C. for 2 h under Ar. The mixture was concentrated and purified by preparative TLC (EtOAc/petroleum ether: 1/2) to give 7-(4-fluoro-2-methoxy-phenyl)-4-methoxy-6-[1-[(4-methoxyphenyl)methyl]pyrazol-4-yl]pyrazolo[1,5-a]pyrazine (82 mg, 74% yield). LCMS ESI (+) m/z 460.2 (M+H).

Step D: Preparation of 7-(4-fluoro-2-methoxy-phenyl)-6-[1-[(4-methoxyphenyl)methyl]pyrazol-4-yl]pyrazolo[1,5-a]pyrazin-4-ol: To a solution of 7-(4-fluoro-2-methoxy-phenyl)-4-methoxy-6-[1-[(4-methoxyphenyl)methyl]pyrazol-4-yl]pyrazolo[1,5-a]pyrazine (82 mg, 0.18 mmol) in acetonitrile (4 mL) was added sodium iodide (134 mg, 0.892 mmol) and trimethylchlorosilane (0.11 mL, 0.89 mmol) at 0° C. The mixture was stirred at ambient temperature for 2 h. The mixture was diluted with water and quenched with Na₂SO₃ aqueous solution. The product was extracted with EtOAc, washed with brine, dried over Na₂SO₄ and concentrated to dryness to give the crude 7-(4-fluoro-2-methoxy-phenyl)-6-[1-[(4-methoxyphenyl)methyl]pyrazol-4-yl]pyrazolo[1,5-a]pyrazin-4-ol (81 mg, 92% yield), which was used in the next step without further purification.

Step E: Preparation of 4-chloro-7-(4-fluoro-2-methoxy-phenyl)-6-[1-[(4-methoxyphenyl)methyl]pyrazol-4-yl]pyrazolo[1,5-a]pyrazine: A suspension of 7-(4-fluoro-2-methoxy-phenyl)-6-[1-[(4-methoxyphenyl)methyl]pyrazol-4-yl]pyrazolo[1,5-a]pyrazin-4-ol (80 mg, 0.16 mmol) in phosphorus oxychloride (1.00 mL, 10.7 mmol) was stirred at 90° C. for 3 h. The mixture was concentrated and diluted with EtOAc. The mixture was washed with aqueous NaHCO₃ and brine solution. The organic phase was dried over Na₂SO₄, concentrated and purified by preparative TLC (EtOAc/petroleum ether: 1/3) to give 4-chloro-7-(4-fluoro-2-methoxy-phenyl)-6-[1-[(4-methoxyphenyl)methyl]pyrazol-4-yl]pyrazolo[1,5-a]pyrazine (62 mg, 83% yield). LCMS ESI (+) m/z 464.1 (M+H).

Step F: Preparation of 4-chloro-7-(4-fluoro-2-methoxy-phenyl)-6-(1H-pyrazol-4-yl)pyrazolo[1,5-a]pyrazine: A solution of 4-chloro-7-(4-fluoro-2-methoxy-phenyl)-6-[1-[(4-methoxyphenyl)methyl]pyrazol-4-yl]pyrazolo[1,5-a]pyrazine (57 mg, 0.12 mmol) in trifluoroacetic acid (5 mL) was stirred at 85° C. in a sealed tube for 4 h. The mixture was concentrated to dryness. Phosphorus oxychloride (4.0 mL) was added to the residue, and the resulting mixture was stirred for additional 2 h at 90° C. The mixture was concentrated to dryness and diluted with EtOAc. The mixture was washed with aqueous NaHCO₃ and brine solution. The organic phase was dried over Na₂SO₄, concentrated and purified by preparative TLC (EtOAc/petroleum ether: 1/2) to give 4-chloro-7-(4-fluoro-2-methoxy-phenyl)-6-(1H-pyrazol-4-yl)pyrazolo[1,5-a]pyrazine (36 mg, 85% yield). LCMS ESI (+) m/z 344.1 (M+H).

Step G: Preparation of tert-butyl 3-[4-[4-chloro-7-(4-fluoro-2-methoxy-phenyl)pyrazolo[1,5-a]pyrazin-6-yl]pyrazol-1-yl]azetidine-1-carboxylate: To a solution of 4-chloro-7-(4-fluoro-2-methoxy-phenyl)-6-(1H-pyrazol-4-yl)pyrazolo[1,5-a]pyrazine (19 mg, 0.055 mmol) and tert-butyl 3-iodoazetidine-1-carboxylate (65 mg, 0.23 mmol) in DMF (2 mL) was added cesium carbonate (92 mg, 0.28 mmol). The mixture was stirred at 55° C. for 0.5 h. The mixture was quenched with brine aqueous solution and the product was extracted with EtOAc/petroleum ether (1/1) twice. The organic phases were dried over anhydrous Na₂SO₄, filtered and concentrated. The residue was purified by preparative TLC (EtOAc/petroleum ether: 1/2) to give tert-butyl 3-[4-[4-chloro-7-(4-fluoro-2-methoxy-phenyl)pyrazolo[1,5-a]pyrazin-6-yl]pyrazol-1-yl]azetidine-1-carboxylate (14 mg, 51% yield). LCMS ESI (+) m/z 499.2 (M+H).

Step H: Preparation of 6-[1-(azetidin-3-yl)pyrazol-4-yl]-4-chloro-7-(4-fluoro-2-methoxy-phenyl)pyrazolo[1,5-a]pyrazine; 2,2,2-trifluoroacetic acid: To a solution of tert-butyl 3-[4-[4-chloro-7-(4-fluoro-2-methoxy-phenyl)pyrazolo[1,5-a]pyrazin-6-yl]pyrazol-1-yl]azetidine-1-carboxylate (14 mg, 0.028 mmol) in DCM (2 mL) was added trifluoroacetic acid (0.50 mL). The mixture was stirred at 20° C. for 1 h. The mixture was concentrated to dryness to give 6-[1-(azetidin-3-yl)pyrazol-4-yl]-4-chloro-7-(4-fluoro-2-methoxy-phenyl)pyrazolo[1,5-a]pyrazine as a trifluoroacetic acid salt (18 mg, 100% yield). LCMS ESI (+) m/z 399.1 (M+H).

Step I: Preparation of 1-[3-[4-[4-chloro-7-(4-fluoro-2-methoxy-phenyl)pyrazolo[1,5-a]pyrazin-6-yl]pyrazol-1-yl]azetidin-1-yl]prop-2-en-1-one: To a solution of 6-[1-(azetidin-3-yl)pyrazol-4-yl]-4-chloro-7-(4-fluoro-2-methoxy-phenyl)pyrazolo[1,5-a]pyrazine trifluoroacetic acid salt (18 mg, 0.028 mmol) and triethylamine (0.029 mL, 0.21 mmol) in DCM (3 mL) was added acryloyl chloride (0.0045 mL, 0,055 mmol) at −60° C. The mixture was stirred at −60° C. for 15 min. The mixture was poured into NaHCO₃ aqueous solution and extracted with EtOAc. The combined organic layers were washed with brine, dried over anhydrous Na₂SO₄ and concentrated to dryness to give 1-[3-[4-[4-chloro-7-(4-fluoro-2-methoxy-phenyl)pyrazolo[1,5-a]pyrazin-6-yl]pyrazol-1-yl]azetidin-1-yl]prop-2-en-1-one (12 mg, 94% yield) which was used in the next step directly. LCMS ESI (+) m/z 453.1 (M+H).

Step J: Preparation of tert-butyl 6-[7-(4-fluoro-2-methoxy-phenyl)-6-[1-(1-prop-2-enoylazetidin-3-yl)pyrazol-4-yl]pyrazolo[1,5-a]pyrazin-4-yl]-3,4-dihydro-1H-isoquinoline-2-carboxylate: To a solution of 1-[3-[4-[4-chloro-7-(4-fluoro-2-methoxy-phenyl)pyrazolo[1,5-a]pyrazin-6-yl]pyrazol-1-yl]azetidin-1-yl]prop-2-en-1-one (12 mg, 0.027 mmol), sodium carbonate (8.0 mg, 0.076 mmol), and tert-butyl 6-(4,4,5,5-tetramethyl-1,3,2-dioxaborolan-2-yl)-3,4-dihydro-1H-isoquinoline-2-carboxylate (16 mg, 0.045 mmol) in 1,4-dioxane (2 mL) and water (0.2 mL) was added tetrakis(triphenylphosphine)palladium(0) (3.1 mg, 0.0027 mmol). The mixture was stirred at 85° C. for 1 h under Ar. The mixture was concentrated and purified by preparative TLC (EtOAc/petroleum ether: 2/1) to give tert-butyl 6-[7-(4-fluoro-2-methoxy-phenyl)-6-[1-(1-prop-2-enoylazetidin-3-yl)pyrazol-4-yl]pyrazolo[1,5-a]pyrazin-4-yl]-3,4-dihydro-1H-isoquinoline-2-carboxylate (9.6 mg, 56% yield).

Step K: Preparation of 1-[3-[4-[7-(4-fluoro-2-methoxy-phenyl)-4-(1,2,3,4-tetrahydroisoquinolin-6-yl)pyrazolo[1,5-a]pyrazin-6-yl]pyrazol-1-yl]azetidin-1-yl]prop-2-en-1-one: To a solution of tert-butyl 6-[7-(4-fluoro-2-methoxy-phenyl)-6-[1-(1-prop-2-enoylazetidin-3-yl)pyrazol-4-yl]pyrazolo[1,5-a]pyrazin-4-yl]-3,4-dihydro-1H-isoquinoline-2-carboxylate (9.6 mg, 0.015 mmol) in DCM (2 mL) was added trifluoroacetic acid (0.50 mL, 6.5 mmol). The mixture was stirred at 25° C. for 1 h. The mixture was concentrated and purified by reverse phase preparative HPLC to give 1-[3-[4-[7-(4-fluoro-2-methoxy-phenyl)-4-(1,2,3,4-tetrahydroisoquinolin-6-yl)pyrazolo[1,5-a]pyrazin-6-yl]pyrazol-1-yl]azetidin-1-yl]prop-2-en-1-one (6.0 mg, 74% yield) as a bis trifluoroacetic acid salt. LCMS ESI (+) m/z 550.2 (M+H). ¹H NMR (400 MHz, CD₃OD) δ 8.06-8.10 (m, 2H), 8.02 (d, J=2.4 Hz, 1H), 7.67 (s, 1H), 7.48 (d, J=8.0 Hz, 1H), 7.38 (s, 1H), 7.32-7.36 (m, 1H), 7.15 (d, J=2.4 Hz, 1H), 7.10 (dd, J=11.2, 2.4 Hz, 1H), 6.90-6.96 (m, 1H), 6.24-6.40 (m, 2H), 5.77 (dd, J=10.0, 2.2 Hz, 1H), 5.20-5.28 (m, 1H), 4.73 (t, J=8.8 Hz, 1H), 4.45-4.60 (m, 4H), 4.29-4.35 (m, 1H), 3.71 (s, 3H), 3.61 (t, J=6.4 Hz, 2H).

### Synthetic Example 29: Synthesis of 1-[4-[4-[7-(4-fluoro-2-methoxy-phenyl)-4-(1,2,3,4-tetrahydroisoquinolin-6-yl)thieno[3,2-c]pyridin-6-yl]pyrazol-1-yl]-4-(hydroxymethyl)-1-piperidyl]prop-2-en-1-one (Compound 180)

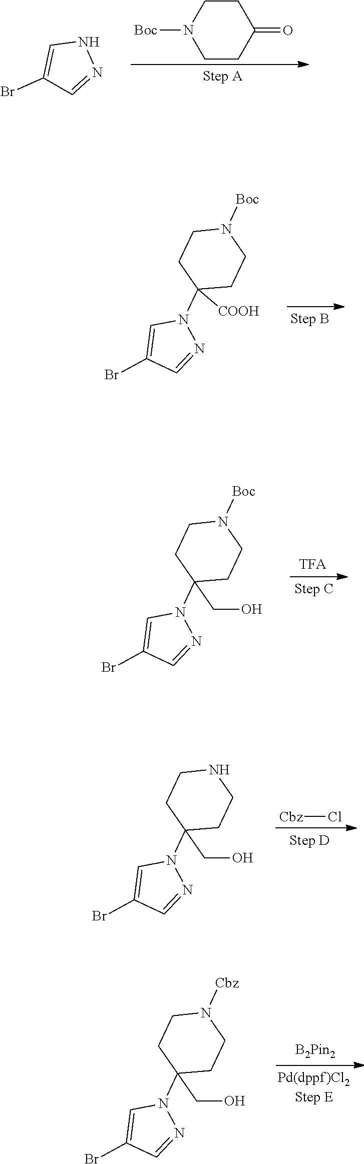

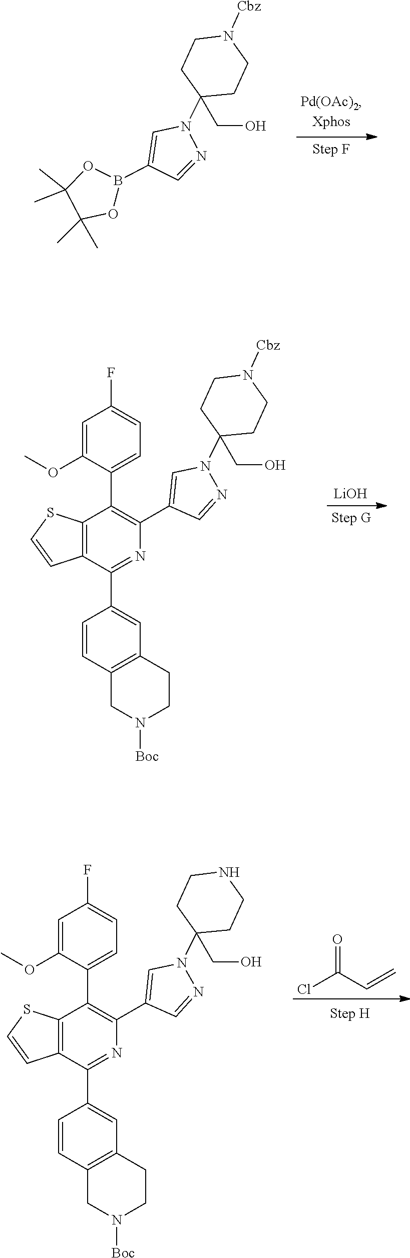

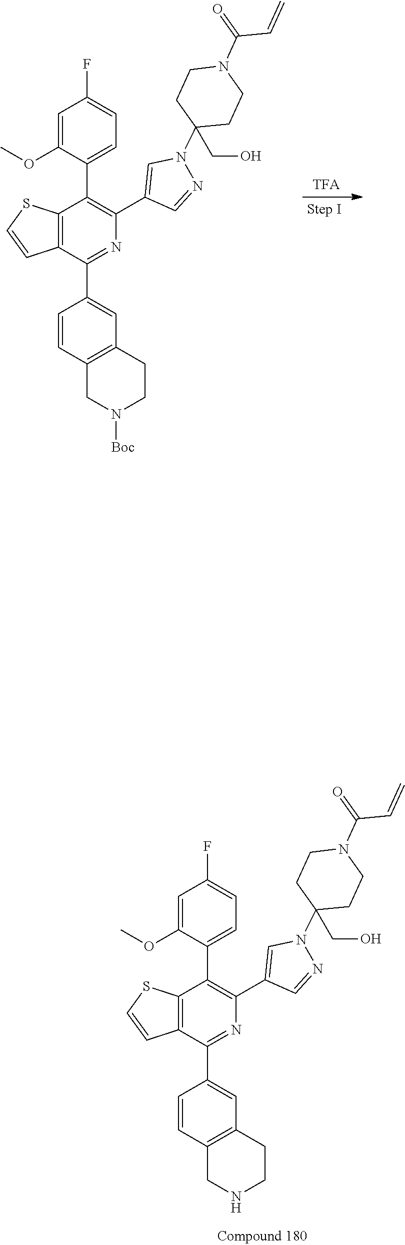

Step A: Preparation of 4-(4-bromopyrazol-1-yl)-1-tert-butoxycarbonyl-piperidine-4-carboxylic acid: To a solution of 4-bromo-1H-pyrazole (5.00 g, 34.0 mmol), 1-Boc-4-piperidone (20.3 g, 102 mmol) and chloroform (20.3 g, 170 mmol) in THF (200 mL) was added NaOH (6.80 g, 170 mmol). The resulting mixture was stirred at 25° C. for 3 hours. The solvent was removed under reduced pressure. To the mixture was added water. 1 N HCl was added to adjust the pH to 4. The mixture was filtered, and the filter cake was dried to afford 4-(4-bromopyrazol-1-yl)-1-tert-butoxycarbonyl-piperidine-4-carboxylic acid (8.30 g, 65% yield) as a white solid. LCMS ESI (+) m/z 374.0 (M+H).

Step B: Preparation of tert-butyl 4-(4-bromopyrazol-1-yl)-4-(hydroxymethyl)piperidine-1-carboxylate: To a solution of 4-(4-bromopyrazol-1-yl)-1-tert-butoxycarbonyl-piperidine-4-carboxylic acid (2.00 g, 5.34 mmol) in THF (20 mL) was added BH₃-THF (1M solution, 16 mL, 16.0 mmol) at 0° C. under Ar atmosphere. The resulting mixture was stirred at 0° C. for 3 hours. Methanol was dripped into the solution slowly until bubbles ceased to be evolved. The solution was washed with 100 mL of saturated sodium bicarbonate solution and extracted twice with ethyl acetate. The organic layers were dried over Na₂SO₄ and concentrated in vacuo to give tert-butyl 4-(4-bromopyrazol-1-yl)-4-(hydroxymethyl) piperidine-1-carboxylate (1.80 g, 93% yield) as a white solid. LCMS ESI (+) m/z 360.1 (M+H).

Step C: Preparation of [4-(4-bromopyrazol-1-yl)-4-piperidyl]methanol: To a solution of tert-butyl 4-(4-bromopyrazol-1-yl)-4-(hydroxymethyl) piperidine-1-carboxylate (1.80 g, 5.00 mmol) in DCM (18 mL) was added trifluoroacetic acid (6.00 mL, 77.9 mmol). The resulting solution was stirred at 25° C. for 1 hour. The resulting mixture was concentrated to dryness to afford [4-(4-bromopyrazol-1-yl)-4-piperidyl]methanol (2.10 g, crude) as a yellow oil that was used directly without further purification. LCMS ESI (+) m/z 260 (M+H).

Step D: Preparation of benzyl 4-(4-bromopyrazol-1-yl)-4-(hydroxymethyl)piperidine-1-carboxylate: To a solution of [4-(4-bromopyrazol-1-yl)-4-piperidyl]methanol (1.60 g, 6.15 mmol) and benzyl chloroformate (1.10 mL, 7.38 mmol) in water (20 mL) was added Na₂CO₃ (2.61 g, 24.6 mmol). The resulting mixture was stirred at 25° C. for 12 hours. The reaction mixture was diluted with EtOAc, washed with water and brine, dried over Na₂SO₄ and evaporated. The residue was purified by column chromatography on silica gel (EtOAc/petroleum ether: 1/2 to 1/1) to afford benzyl 4-(4-bromopyrazol-1-yl)-4-(hydroxymethyl) piperidine-1-carboxylate (1.22 g, 50% yield) as a white solid. LCMS ESI (+) m/z 394.1 (M+H).

Step E: Preparation of benzyl4-(hydroxymethyl)-4-[4-(4,4,5,5-tetramethyl-1,3,2-dioxaborolan-2-yl)pyrazol-1-yl]piperidine-1-carboxylate: To a solution of benzyl 4-(4-bromopyrazol-1-yl)-4-(hydroxymethyl)piperidine-1-carboxylate (180 mg, 0.457 mmol), bis(pinacolato)diboron (174 mg, 0.685 mmol) and KOAc (89 mg, 0.91 mmol) in 1,4-dioxane (2 mL) was added Pd(dppf)Cl₂ (33 mg, 0.046 mmol) under Ar atmosphere. The resulting mixture was stirred at 100° C. for 10 hours. The solvent was removed under reduced pressure, and the residue was purified by preparative TLC (EtOAc/petroleum ether: 1/1) to give benzyl 4-(hydroxymethyl)-4-[4-(4,4,5,5-tetramethyl-1,3,2-dioxaborolan-2-yl)pyrazol-1-yl]piperidine-1-carboxylate (60 mg, 12% yield) as a white solid. LCMS ESI (+) m/z 442.3 (M+H).

Step F: Preparation of tert-butyl 6-[6-[1-[1-benzyloxycarbonyl-4-(hydroxymethyl)-4-piperidyl]pyrazol-4-yl]-7-(4-fluoro-2-methoxy-phenyl)thieno[3,2-c]pyridin-4-yl]-3,4-dihydro-1H-isoquinoline-2-carboxylate: To a solution of tert-butyl 6-[7-(4-fluoro-2-methoxy-phenyl)-6-(trifluoromethyl-sulfonyloxy)thieno[3,2-c]pyridin-4-yl]-3,4-dihydro-1H-isoquinoline-2-carboxylate (33 mg, 0.052 mmol), benzyl 4-(hydroxymethyl)-4-[4-(4,4,5,5-tetramethyl-1,3,2-dioxaborolan-2-yl) pyrazol-1-yl]piperidine-1-carboxylate (46 mg, 0.10 mmol) and K₂CO₃ (14 mg, 0.10 mmol) in acetonitrile (1 mL) and water (0.1 mL) were added Pd(OAc)₂ (1.2 mg, 0.0052 mmol) and XPhos (4.9 mg, 0.010 mmol) under Ar atmosphere. The resulting mixture was stirred at 100° C. for 1 hour. The solvent was removed under reduced pressure, and the residue was purified by preparative TLC (EtOAc/petroleum ether: 2/1) to give tert-butyl 6-[6-[1-[1-benzyloxycarbonyl-4-(hydroxymethyl)-4-piperidyl]pyrazol-4-yl]-7-(4-fluoro-2-methoxy-phenyl)thieno[3,2-c]pyridin-4-yl]-3,4-dihydro-1H-isoquinoline-2-carboxylate (20 mg, 48% yield) as a yellow solid. LCMS ESI (+) m/z 804.4 (M+H).

Step G: Preparation of tert-butyl 6-[7-(4-fluoro-2-methoxy-phenyl)-6-[1-[4-(hydroxymethyl)-4-piperidyl]pyrazol-4-yl]thieno[3,2-c]pyridin-4-yl]-3,4-dihydro-1H-isoquinoline-2-carboxylate: To a solution of tert-butyl 6-[6-[1-[1-benzyloxycarbonyl-4-(hydroxymethyl)-4-piperidyl]pyrazol-4-yl]-7-(4-fluoro-2-methoxy-phenyl)thieno[3,2-c]pyridin-4-yl]-3,4-dihydro-1H-isoquinoline-2-carboxylate (20 mg, 0.025 mmol) in 1,4-dioxane (0.5 mL) and water (0.5 mL) was added lithium hydroxide monohydrate (50 mg, 1.3 mmol). The mixture was stirred at 100° C. for 48 hours. The reaction mixture was diluted with water (10 mL), extracted with DCM (3×10 mL) and washed with brine (10 mL). The organic layer was dried over Na₂SO₄, filtered and evaporated to afford tert-butyl 6-[7-(4-fluoro-2-methoxy-phenyl)-6-[1-[4-(hydroxymethyl)-4-piperidyl]pyrazol-4-yl]thieno[3,2-c]pyridin-4-yl]-3,4-dihydro-1H-isoquinoline-2-carboxylate (25 mg, crude) as a yellow solid that was used directly without further purification. LCMS ESI (+) m/z 670.4 (M+H).

Step H: Preparation of tert-butyl 6-[7-(4-fluoro-2-methoxy-phenyl)-6-[1-[4-(hydroxymethyl)-1-prop-2-enoyl-4-piperidyl]pyrazol-4-yl]thieno[3,2-c]pyridin-4-yl]-3,4-dihydro-1H-isoquinoline-2-carboxylate: To a solution of tert-butyl 6-[7-(4-fluoro-2-methoxy-phenyl)-6-[1-[4-(hydroxymethyl)-4-piperidyl]pyrazol-4-yl]thieno[3,2-c]pyridin-4-yl]-3,4-dihydro-1H-isoquinoline-2-carboxylate (25 mg, 0.024 mmol) and DIEA (0.0086 mL, 0.049 mmol) in DCM (2 mL) was added acryloyl chloride (0.0030 mL, 0.036 mmol) at −60° C. under Ar. The resulting mixture was stirred at −60° C. for 1 hour. The reaction mixture was poured into saturated NaHCO₃ solution (10 mL). The organic layer was washed with brine, dried over anhydrous sodium sulfate, filtered and concentrated to give tert-butyl 6-[7-(4-fluoro-2-methoxy-phenyl)-6-[1-[4-(hydroxymethyl)-1-prop-2-enoyl-4-piperidyl]pyrazol-4-yl]thieno[3,2-c]pyridin-4-yl]-3,4-dihydro-1H-isoquinoline-2-carboxylate (10 mg, 57% yield) as a white solid. LCMS ESI (+) m/z 724.4 (M+H).

Step I: Preparation of 1-[4-[4-[7-(4-fluoro-2-methoxy-phenyl)-4-(1,2,3,4-tetrahydroisoquinolin-6-yl)thieno[3,2-c]pyridin-6-yl]pyrazol-1-yl]-4-(hydroxymethyl)-1-piperidyl]prop-2-en-1-one: To a solution of tert-butyl 6-[7-(4-fluoro-2-methoxy-phenyl)-6-[1-[4-(hydroxymethyl)-1-prop-2-enoyl-4-piperidyl]pyrazol-4-yl]thieno[3,2-c]pyridin-4-yl]-3,4-dihydro-1H-isoquinoline-2-carboxylate (10 mg, 0.014 mmol) in DCM (2 mL) was added trifluoroacetic acid (0.70 mL). The resulting solution was stirred at 20° C. for 1 hour. The mixture was concentrated and purified by reverse phase preparative HPLC to give 1-[4-[4-[7-(4-fluoro-2-methoxy-phenyl)-4-(1,2,3,4-tetrahydroisoquinolin-6-yl)thieno[3,2-c]pyridin-6-yl]pyrazol-1-yl]-4-(hydroxymethyl)-1-piperidyl]prop-2-en-1-one (7.6 mg, 88% yield) as a bis trifluoroacetic acid salt. LCMS ESI (+) m/z 624.3 (M+H). ¹H NMR (400 MHz, CD₃OD) δ 7.83-7.89 (m, 2H), 7.75-7.81 (m, 1H), 7.63 (d, J=5.4 Hz, 2H), 7.59 (s, 1H), 7.49 (d, J=7.8 Hz, 1H), 7.32-7.40 (m, 1H), 7.04 (dd, J=11.0, 2.2 Hz, 1H), 6.89-6.96 (m, 1H), 6.76 (dd, J=16.8, 10.7 Hz, 1H), 6.18 (dd, J=16.8, 1.8 Hz, 1H), 5.74 (dd, J=10.6, 1.8 Hz, 1H), 4.52 (s, 2H), 4.27 (d, J=13.4 Hz, 1H), 3.93 (d, J=13.5 Hz, 1H), 3.66 (s, 3H), 3.61 (t, J=6.4 Hz, 2H), 3.55 (s, 2H), 2.97-3.10 (m, 1H), 2.68-2.80 (m, 1H), 2.31-2.48 (m, 2H), 1.80-1.95 (m, 2H).

### Synthetic Example 30: Synthesis of 1-[2-[7-[4-fluoro-2-(2-methoxyethoxy)phenyl]-4-(2-isopropyl-3,4-dihydro-1H-isoquinolin-6-yl)thieno[3,2-c]pyridin-6-yl]-6,8-dihydro-5H-[1,24]triazolo[1,5-a]pyrazin-7-yl]prop-2-en-1-one (Compound 185)

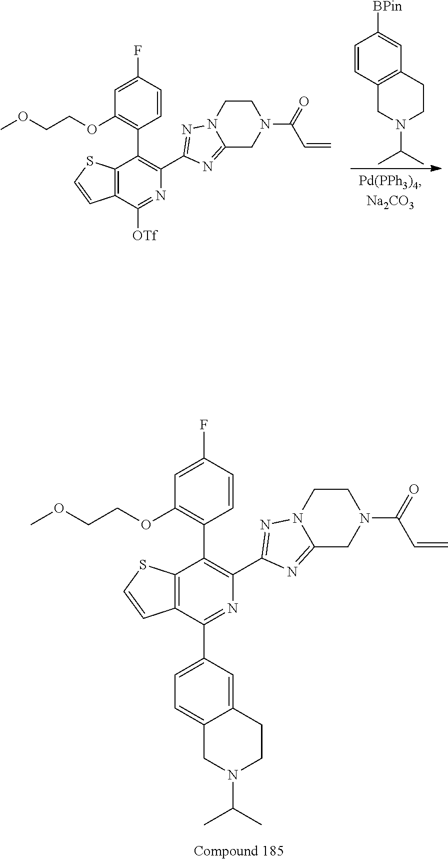

To a solution of 6-(7-acryloyl-5,6,7,8-tetrahydro-[1,2,4]triazolo[1,5-a]pyrazin-2-yl)-7-(4-fluoro-2-(2-methoxyethoxy)phenyl)thieno[3,2-c]pyridin-4-yl trifluoromethanesulfonate (50 mg, 0.080 mmol), sodium carbonate (17 mg, 0.16 mmol) and 2-isopropyl-6-(4,4,5,5-tetramethyl-1,3,2-dioxaborolan-2-yl)-3,4-dihydro-1H-isoquinoline (36 mg, 0.12 mmol) in 1,4-dioxane (2 mL) and water (0.2 mL) was added tetrakis(triphenylphosphine)palladium(0) (9.2 mg, 0.0080 mmol). The mixture was stirred at 100° C. for 2 h under Ar. The mixture was poured into water. The product was extracted with EtOAc, dried over anhydrous Na₂SO₄, concentrated and purified by reverse phase preparative HPLC to give 1-[2-[7-[4-fluoro-2-(2-methoxyethoxy)phenyl]-4-(2-isopropyl-3,4-dihydro-1H-isoquinolin-6-yl)thieno[3,2-c]pyridin-6-yl]-6,8-dihydro-5H-[1,2,4]triazolo[1,5-a]pyrazin-7-yl]prop-2-en-1-one (24 mg, 45% yield) as a bis trifluoroacetic acid salt. LCMS ESI (+) m/z 653.3 (M+H). ¹H NMR (400 MHz, CD₃OD) δ ¹H NMR (400 MHz, CD₃OD) δ 7.95 (s, 1H), 7.92 (d, J=8.08 Hz, 1H), 7.86 (d, J=5.6 Hz, 1H), 7.70 (d, J=5.6 Hz, 1H), 7.48 (d, J=8.0 Hz, 1H), 7.35 (t, J=7.4 Hz, 1H), 6.79-6.92 (m, 3H), 6.30 (dd, J=16.8, 1.4 Hz, 1H), 5.85 (dd, J=10.6, 1.8 Hz, 1H), 4.91-5.00 (m, 2H), 4.59 (s, 2H), 4.11-4.17 (m, 4H), 3.98-4.04 (m, 1H), 3.77-3.88 (m, 3H), 3.32-3.55 (m, 5H), 3.06 (s, 3H), 1.51 (d, J=6.6 Hz, 6H).

### Synthetic Example 31: Synthesis of 6-[7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]-3,4-dihydro-2H-isoquinolin-1-one (Compound 189)

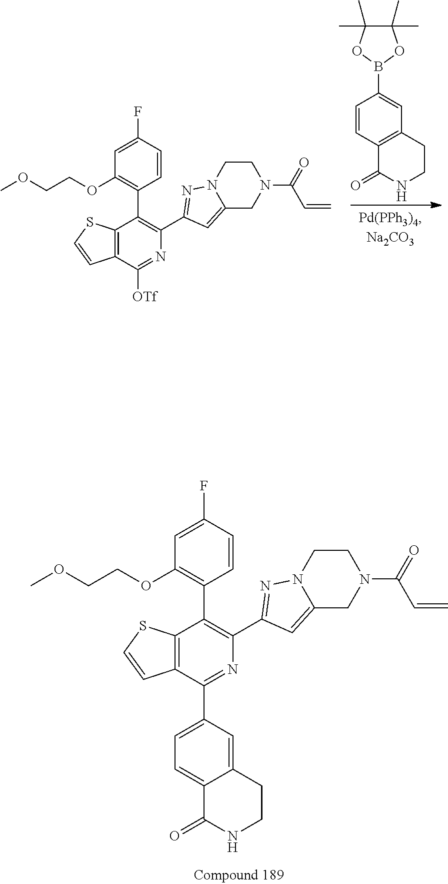

To a stirred solution of 6-(4,4,5,5-tetramethyl-1,3,2-dioxaborolan-2-yl)-3,4-dihydro-2H-isoquinolin-1-one (68 mg, 0.25 mmol) and [7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]trifluoromethanesulfonate (50 mg, 0.080 mmol) in 1,4-dioxane (5 mL) and water (0.2 mL) were added Na₂CO₃ (6.6 mg, 0.062 mmol) and Pd(PPh₃)₄(3.6 mg, 0.0031 mmol). The mixture was stirred at 100° C. for 2 h under Ar. The mixture was poured into water. The product was extracted with EtOAc, dried over anhydrous Na₂SO₄, concentrated and purified by reverse phase preparative HPLC to give 6-[7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]-3,4-dihydro-2H-isoquinolin-1-one (29 mg, 51% yield) as a trifluoroacetic acid salt. LCMS ESI (+) m/z 624.3 (M+H). ¹H NMR (400 MHz, CD₃OD) δ 8.23 (d, J=7.9 Hz, 1H), 8.09 (d, J=5.6 Hz, 1H), 7.93-7.98 (m, 2H), 7.81 (d, J=5.6 Hz, 1H), 7.41 (dd, J=8.3, 6.6 Hz, 1H), 7.09 (dd, J=11.0, 2.3 Hz, 1H), 6.70-6.98 (m, 2H), 6.28 (d, J=15.7 Hz, 1H), 5.83 (s, 2H), 4.70-4.86 (m, 2H), 4.28 (s, 2H), 4.06-4.16 (m, 3H), 3.95-4.04 (m, 1H), 3.61 (t, J=6.6 Hz, 2H), 3.42 (t, J=4.5 Hz, 2H), 3.17 (t, J=6.6 Hz, 2H), 3.09 (s, 3H).

### Synthetic Example 32: Synthesis of 1-[2-[7-[4-fluoro-2-(2-methoxyethoxy)phenyl]-4-(2-isopropyl-3,4-dihydro-1H-isoquinolin-6-yl)thieno[3,2-c]pyridin-6-yl]-6,7-dihydro-4H-pyrazolo[1,5-a]pyrazin-5-yl]prop-2-en-1-one (Compound 190)

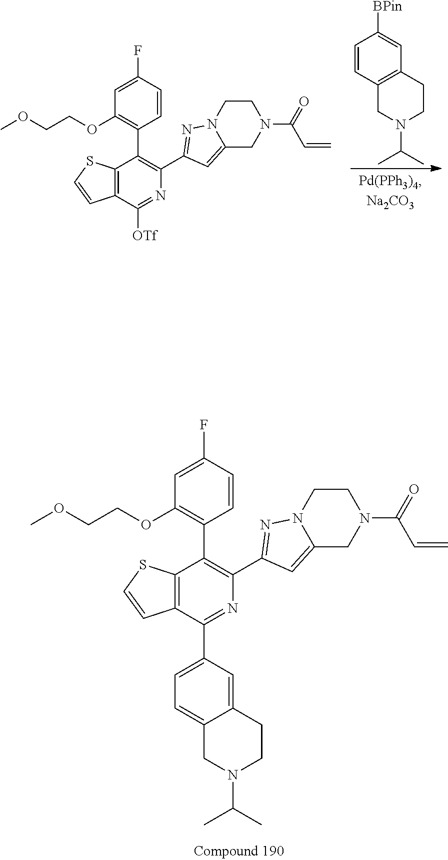

To a solution of [7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]trifluoromethanesulfonate (40 mg, 0.064 mmol) in 1,4-dioxane (1 mL) and water (0.2 mL) were added 2-isopropyl-6-(4,4,5,5-tetramethyl-1,3,2-dioxaborolan-2-yl)-3,4-dihydro-1H-isoquinoline (25 mg, 0.083 mmol), sodium carbonate (14 mg, 0.13 mmol) and Pd(PPh₃)₄ (7.4 mg, 0.0064 mmol) under Ar. The mixture was stirred at 95° C. for 3 hours. The reaction was concentrated to dryness. The residue was taken up in EtOAc, washed with water and brine, dried (MgSO₄) and concentrated. The crude was then purified by reverse phase preparative HPLC to give the 1-[2-[7-[4-fluoro-2-(2-methoxyethoxy)phenyl]-4-(2-isopropyl-3,4-dihydro-1H-isoquinolin-6-yl)thieno[3,2-c]pyridin-6-yl]-6,7-dihydro-4H-pyrazolo[1,5-a]pyrazin-5-yl]prop-2-en-1-one (19 mg, 45% yield) as a bis trifluoroacetic acid salt. LCMS ESI (+) m/z 652.2 (M+H). ¹H NMR (400 MHz, CD₃OD) δ 7.87-7.98 (m, 3H), 7.67-7.74 (m, 1H), 7.50-7.57 (m, 1H), 7.31-7.39 (m, 1H), 6.99-7.07 (m, 1H), 6.75-6.92 (m, 2H), 6.28 (d, J=15.5 Hz, 1H), 5.79-5.92 (m, 2H), 4.73-4.80 (m, 1H), 4.61 (s, 2H), 4.21-4.30 (m, 2H), 4.03-4.16 (m, 3H), 3.92-4.00 (m, 1H), 3.75-3.89 (m, 2H), 3.34-3.55 (m, 5H), 3.07 (s, 3H), 1.51 (d, J=6.7 Hz, 6H).

### Synthetic Example 33: Synthesis of 1-[2-[7-[4-fluoro-2-(2-methoxyethoxy)phenyl]-4-(1-methylpyrazol-4-yl)thieno[3,2-c]pyridin-6-yl]-6,7-dihydro-4H-pyrazolo[1,5-a]pyrazin-5-yl]prop-2-en-1-one (Compound 194)

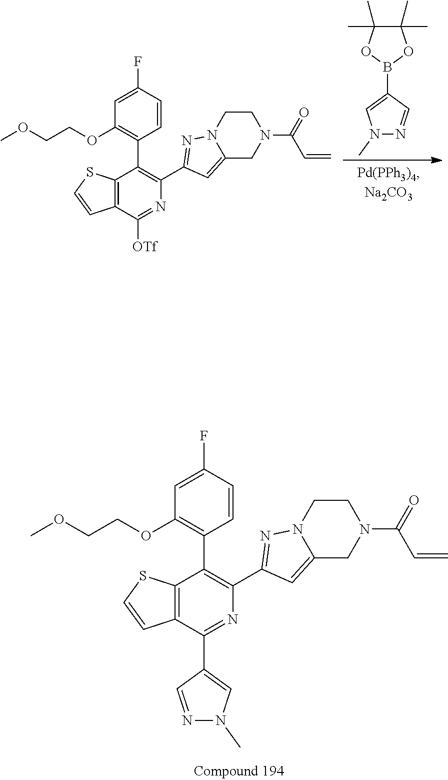

To a solution of [7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]trifluoromethanesulfonate (40 mg, 0.064 mmol), 1-methyl-4-(4,4,5,5-tetramethyl-1,3,2-dioxaborolan-2-yl)pyrazole (20 mg, 0.096 mmol) and sodium carbonate (14 mg, 0.13 mmol) in 1,4-dioxane (0.8 mL)/water (0.1 mL) was added tetrakis(triphenylphosphine)palladium (7.4 mg, 0.0064 mmol). The mixture was stirred at 100° C. under argon for 2 h. The reaction was concentrated to dryness and the residue was taken up in ACN (1 mL). The crude was filtered and purified by reverse phase preparative HPLC to give 1-[2-[7-[4-fluoro-2-(2-methoxyethoxy)phenyl]-4-(1-methylpyrazol-4-yl)thieno[3,2-c]pyridin-6-yl]-6,7-dihydro-4H-pyrazolo[1,5-a]pyrazin-5-yl]prop-2-en-1-one (30 mg, 85% yield) as a trifluoroacetic acid salt, LCMS ESI (+) m/z 559.2 (M+H). ¹H NMR (400 MHz, CD₃OD) δ 8.68 (s, 1H), 8.35 (s, 1H), 8.16 (d, J=6.0 Hz, 1H), 8.09 (d, J=5.6 Hz, 1H), 7.41 (dd, J=8.4, 6.4 Hz, 1H), 7.10 (dd, J=11.2, 2.4 Hz, 1H), 6.85-6.97 (m, 2H), 6.29 (d, J=16.4 Hz, 1H), 5.81-5.85 (m, 2H), 4.7-4.86 (m, 2H), 4.35 (s, 2H), 4.14-4.17 (m, 2H), 4.11 (s, 3H), 4.06-4.11 (m, 1H), 3.98-4.03 (m, 1H), 3.40 (t, J=4.4 Hz, 2H), 3.07 (s, 3H).

### Synthetic Example 34: Synthesis of 5-[7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]-1-methyl-pyridin-2-one (Compound 195)

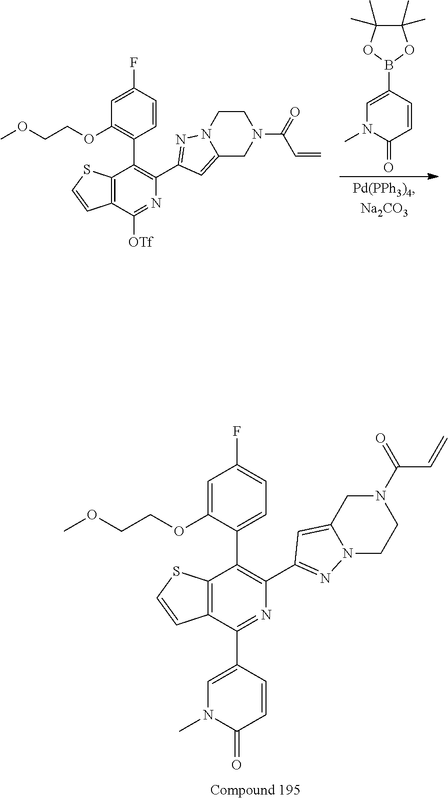

To a solution of [7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]trifluoromethanesulfonate (40 mg, 0.064 mmol), 1-methyl-5-(4,4,5,5-tetramethyl-1,3,2-dioxaborolan-2-yl)pyridin-2-one (23 mg, 0.096 mmol) and sodium carbonate (14 mg, 0.13 mmol) in 1,4-dioxane (0.8 mL)/water (0.1 mL) was added tetrakis(triphenylphosphine) palladium (7.4 mg, 0.0064 mmol). The mixture was stirred at 100° C. under argon for 2 h. The reaction was concentrated to dryness and the residue was taken up in ACN (1 mL). The crude was filtered and purified by reverse phase preparative HPLC to give 5-[7-[4-fluoro-2-(2-methoxyethoxy)phenyl]-6-(5-prop-2-enoyl-6,7-dihydro-4H-pyrazolo[1,5-a]pyrazin-2-yl)thieno[3,2-c]pyridin-4-yl]-1-methyl-pyridin-2-one (21 mg, 56% yield) as a trifluoroacetic acid salt. LCMS ESI (+) m/z 586.2 (M+H). ¹H NMR (400 MHz, CD₃OD) δ 8.54 (d, J=2.4 Hz, 1H), 8.13 (dd, J=9.6, 2.8 Hz, 1H), 8.06 (d, J=5.6 Hz, 1H), 7.90 (d, J=5.6 Hz, 1H), 7.37 (dd, J=8.4, 6.8 Hz, 1H), 7.07 (dd, J=10.8, 2.4 Hz, 1H), 6.90-6.95 (m, 1H), 6.77 (d, J=9.4 Hz, 2H), 6.28 (d, J=15.6 Hz, 1H), 5.81-5.85 (m, 2H), 4.75-4.87 (m, 2H), 4.29 (s, 2H), 4.12-4.15 (m, 2H), 4.05-4.10 (m, 1H), 3.95-4.00 (m, 1H), 3.73 (s, 3H), 3.40 (t, J=4.4 Hz, 2H), 3.08 (s, 3H).

### Synthetic Example 35: Synthesis of 1-[2-[7-[4-fluoro-2-(2-methoxyethoxy)phenyl]-4-(2-oxa-7-azaspiro[3.5]nonan-7-yl)thieno[3,2-c]pyridin-6-yl]-6,7-dihydro-4H-pyrazolo[1,5-a]pyrazin-5-yl]prop-2-en-1-one (Compound 198)

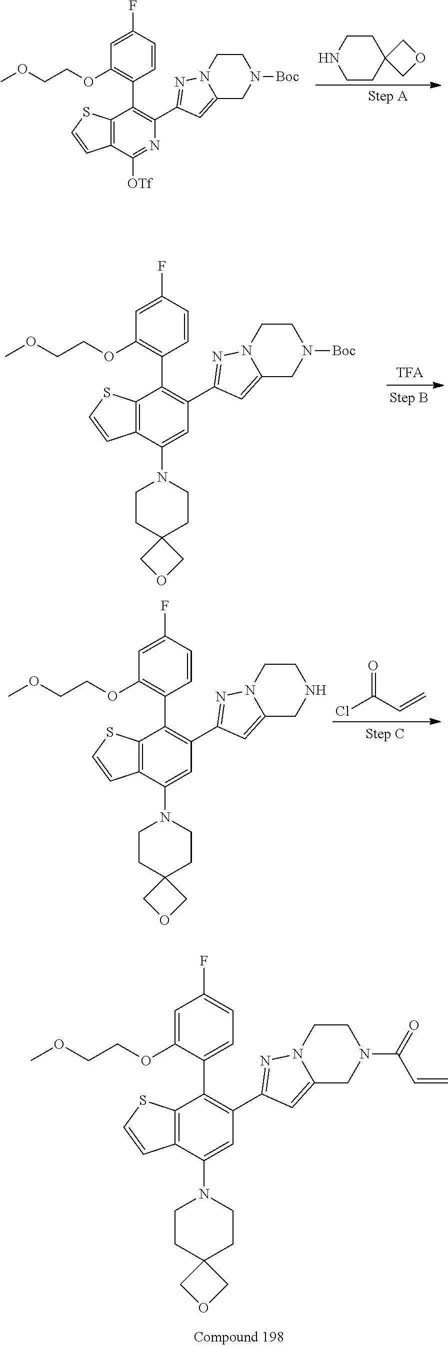

Step A: Preparation of tert-butyl 2-[7-[4-fluoro-2-(2-methoxyethoxy)phenyl]-4-(2-oxa-7-azaspiro[3.5]nonan-7-yl)thieno[3,2-c]pyridin-6-yl]-6,7-dihydro-4H-pyrazolo[1,5-a]pyrazine-5-carboxylate: To a solution of tert-butyl 2-[7-[4-fluoro-2-(2-methoxyethoxy)phenyl]-4-(trifluoromethyl-sulfonyloxy)thieno[3,2-c]pyridin-6-yl]-6,7-dihydro-4H-pyrazolo[1,5-a]pyrazine-5-carboxylate (60 mg, 0.089 mmol) and N,N-diisopropylethylamine (0.061 mL, 0.35 mmol) in DMF (2 mL) was added 2-oxa-7-azaspiro[3.5]nonane (105 mg, 0.826 mmol). The solution was stirred at 100° C. for 16 h under Ar. The reaction mixture was diluted with water and extracted with ethyl acetate. The combined organic layers were washed with brine, dried over Na₂SO₄, filtered and concentrated. The residue was purified by preparative TLC (EtOAc) to give tert-butyl 2-[7-[4-fluoro-2-(2-methoxyethoxy)phenyl]-4-(2-oxa-7-azaspiro[3.5]nonan-7-yl)thieno[3,2-c]pyridin-6-yl]-6,7-dihydro-4H-pyrazolo[1,5-a]pyrazine-5-carboxylate (55 mg, 94% yield) as a white solid. LCMS ESI (+) m/z 650.3 (M+H).

Step B: Preparation of 7-(7-(4-fluoro-2-(2-methoxyethoxy)phenyl)-6-(4,5,6,7-tetrahydropyrazolo[1,5-a]pyrazin-2-yl)thieno[3,2-c]pyridin-4-yl)-2-oxa-7-azaspiro[3.5]nonane: To a stirred solution of tert-butyl 2-[7-[4-fluoro-2-(2-methoxyethoxy)phenyl]-4-(2-oxa-7-azaspiro[3.5]nonan-7-yl)thieno[3,2-c]pyridin-6-yl]-6,7-dihydro-4H-pyrazolo[1,5-a]pyrazine-5-carboxylate (50 mg, 0.077 mmol) in DCM (3 mL) was added trifluoroacetic acid (1.0 mL). The solution was stirred at 10° C. for 1 h. The mixture was concentrated to give crude 7-(7-(4-fluoro-2-(2-methoxyethoxy)phenyl)-6-(4,5,6,7-tetrahydropyrazolo[1,5-a]pyrazin-2-yl)thieno[3,2-c]pyridin-4-yl)-2-oxa-7-azaspiro[3.5]nonane (62 mg) which was used in the next step directly. LCMS ESI (+) m/z 550.3 (M+H).

Step C: Preparation of 1-[2-[7-[4-fluoro-2-(2-methoxyethoxy)phenyl]-4-(2-oxa-7-azaspiro[3.5]nonan-7-yl)thieno[3,2-c]pyridin-6-yl]-6,7-dihydro-4H-pyrazolo[1,5-a]pyrazin-5-yl]prop-2-en-1-one: To a stirred solution of 7-[7-[4-fluoro-2-(2-methoxyethoxy)phenyl]-6-(4,5,6,7-tetrahydropyrazolo[1,5-a]pyrazin-2-yl)thieno[3,2-c]pyridin-4-yl]-2-oxa-7-azaspiro[3.5]nonane (41 mg, 0.074 mmol) and triethylamine (0.031 mL, 0.22 mmol) in DCM (1.5 mL) at −60° C. was added acryloyl chloride (0.0091 mL, 0.11 mmol). The mixture was stirred at −60° C. for 10 min. The mixture was concentrated and purified by reverse phase preparative HPLC to give 1-[2-[7-[4-fluoro-2-(2-methoxyethoxy)phenyl]-4-(2-oxa-7-azaspiro[3.5]nonan-7-yl)thieno[3,2-c]pyridin-6-yl]-6,7-dihydro-4H-pyrazolo[1,5-a]pyrazin-5-yl]prop-2-en-1-one (29 mg, 59% yield) as a trifluoroacetic acid salt. LCMS ESI (+) m/z 604.3 (M+H). ¹H NMR (400 MHz, CD₃OD) δ 7.91 (d, J=5.7 Hz, 1H), 7.89 (d, J=5.7 Hz, 1H), 7.26-7.35 (m, 1H), 7.09 (dd, J=11.0, 2.2 Hz, 1H), 6.70-6.95 (m, 2H), 6.28 (d, J=16.6 Hz, 1H), 5.75-5.88 (m, 1H), 5.58 (s, 1H), 4.66-4.85 (m, 2H), 4.60 (s, 4H), 4.31 (s, 2H), 4.11-4.18 (m, 2H), 4.00-4.10 (m, 2H), 3.80-3.96 (m, 4H), 3.40 (t, J=4.5 Hz, 2H), 3.07 (s, 3H), 2.21-2.30 (m, 4H).

### Synthetic Example 36: Synthesis of 6-[7-(4-fluoro-2-isopropoxy-phenyl)-6-(5-prop-2-enoyl-6,7-dihydro-4H-pyrazolo[1,5-a]pyrazin-2-yl)thieno[3,2-c]pyridin-4-yl]-3,4-dihydro-2H-isoquinolin-1-one (Compound 199)

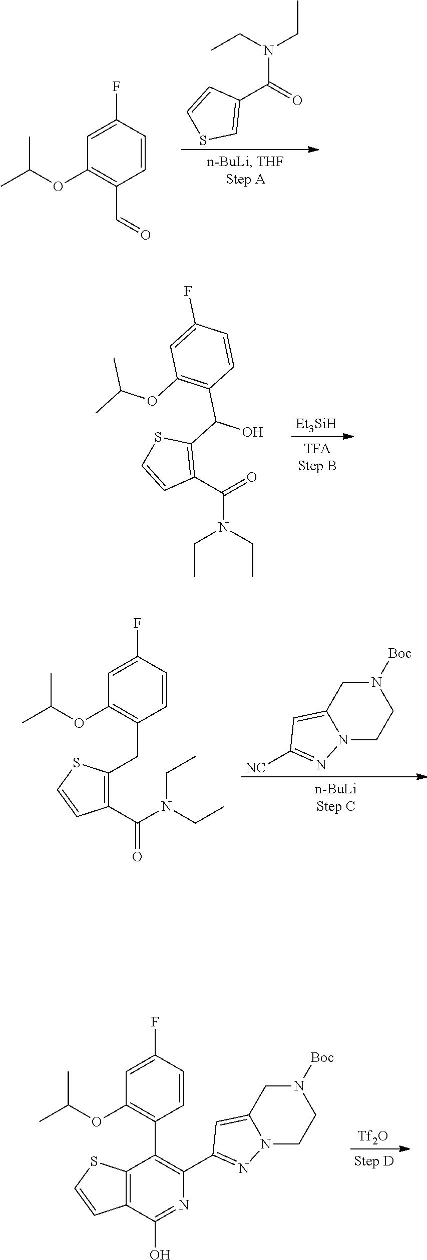

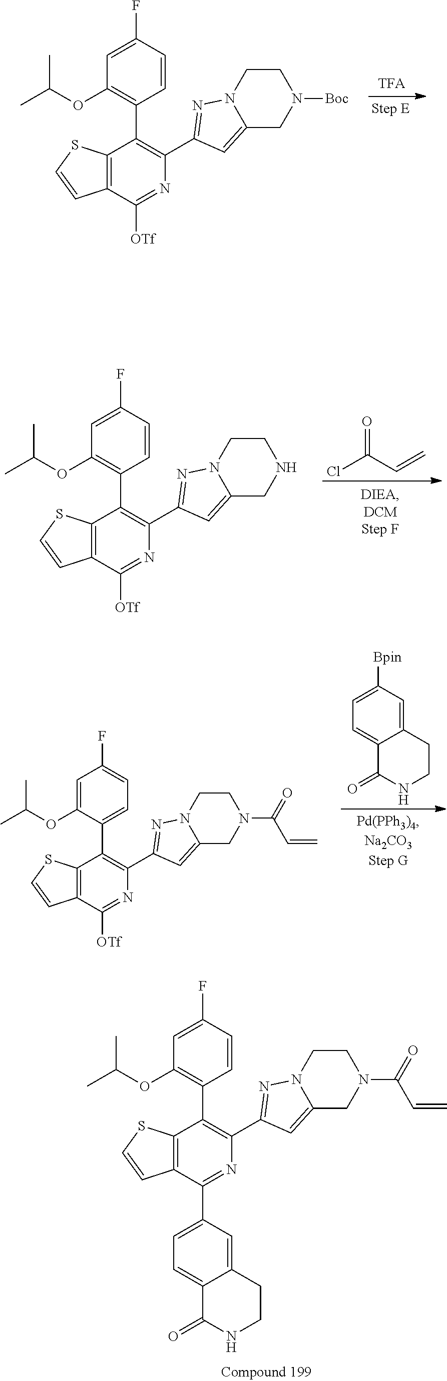

Step A: Preparation of N,N-diethyl-2-[(4-fluoro-2-isopropoxy-phenyl)-hydroxy-methyl]thiophene-3-carboxamide: To a solution of N,N-diethylthiophene-3-carboxamide (8.00 g, 43.7 mmol) in THF (10 mL) was added n-butyllithium (21.0 mL, 2.5 M solution in THF, 52.4 mmol) dropwise at −78° C. The mixture was stirred at this temperature for 30 min. 4-fluoro-2-isopropoxy-benzaldehyde (7.16 g, 39.3 mmol) in THF was added, and the resulting mixture was stirred at −78° C. for 1 hour. The mixture was allowed to warm to ambient temperature. The reaction was quenched by the addition of water, and the product was extracted with EtOAc. The organic phase was dried over anhydrous Na₂SO₄, filtered and concentrated to dryness to give crude N,N-diethyl-2-[(4-fluoro-2-isopropoxy-phenyl)-hydroxy-methyl]thiophene-3-carboxamide (15.5 g, 97% yield) as oil. LCMS ESI (+) m/z 348.2 (M+H).

Step B: Preparation of N,N-diethyl-2-[(4-fluoro-2-isopropoxy-phenyl)methyl]thiophene-3-carboxamide: A mixture of N,N-diethyl-2-[(4-fluoro-2-isopropoxy-phenyl)-hydroxy-methyl]thiophene-3-carboxamide (15.4 g, 42.1 mmol) and triethylsilane (34.0 mL, 211 mmol) in trifluoroacetic acid (80 mL, 1038 mmol) was stirred at 20° C. for 4 hours. The mixture was concentrated. The residue was taken up in EtOAc, washed with NaHCO₃ aqueous solution and brine, dried over Na₂SO₄ and concentrated. The residue was purified by silica gel column (petroleum ether:EtOAc=10:1 to 4:1) to give N,N-diethyl-2-[(4-fluoro-2-isopropoxy-phenyl)methyl]thiophene-3-carboxamide (12.9 g, 88% yield). LCMS ESI (+) m/z 350.2 (M+H).

Step C: Preparation of tert-butyl 2-[7-(4-fluoro-2-isopropoxy-phenyl)-4-hydroxy-thieno[3,2-c]pyridin-6-yl]-6,7-dihydro-4H-pyrazolo[1,5-a]pyrazine-5-carboxylate: To a solution of N,N-diethyl-2-[(4-fluoro-2-isopropoxy-phenyl)methyl]thiophene-3-carboxamide (743 mg, 2.13 mmol) in THF (4 mL) was added n-butyllithium (0.92 mL, 2.5 M in THF, 2.30 mmol) dropwise at −78° C. The mixture was stirred at −78° C. for 30 min. tert-Butyl 2-cyano-6,7-dihydro-4H-pyrazolo[1,5-a]pyrazine-5-carboxylate (440 mg, 1.77 mmol) in THF was added, and the resulting mixture was stirred at ambient temperature for 2 hours. The reaction was quenched by slow addition of water. 1 N HCl was added to adjust the pH to 5-6. The mixture was extracted with EtOAc, dried over Na₂SO₄, filtered and concentrated. The residue was purified by silica gel column (petroleum ether:EtOAc=4:1 to 100% EtOAc) to give tert-butyl 2-[7-(4-fluoro-2-isopropoxy-phenyl)-4-hydroxy-thieno[3,2-c]pyridin-6-yl]-6,7-dihydro-4H-pyrazolo[1,5-a]pyrazine-5-carboxylate (637 mg, 69% yield). LCMS ESI (+) m/z 525.2 (M+H).

Step D: Preparation of tert-butyl 2-[7-(4-fluoro-2-isopropoxy-phenyl)-4-(trifluoromethylsulfonyloxy)thieno[3,2-c]pyridin-6-yl]-6,7-dihydro-4H-pyrazolo[1,5-a]pyrazine-5-carboxylate: To a solution of tert-butyl 2-[7-(4-fluoro-2-isopropoxy-phenyl)-4-hydroxy-thieno[3,2-c]pyridin-6-yl]-6,7-dihydro-4H-pyrazolo[1,5-a]pyrazine-5-carboxylate (637 mg, 1.21 mmol) and pyridine (0.49 mL, 6.1 mmol) in DCM (12 mL) was added trifluoromethanesulfonic anhydride (0.31 mL, 1.8 mmol) at 0° C. The mixture was stirred at 20° C. for 30 min. NaHCO₃ solution was added to quench the reaction. The product was extracted with DCM, dried over Na₂SO₄, filtered, concentrated. The residue was purified by silica gel column (petroleum ether:EtOAc=5:1) to give tert-butyl 2-[7-(4-fluoro-2-isopropoxy-phenyl)-4-(trifluoromethylsulfonyloxy)thieno[3,2-c]pyridin-6-yl]-6,7-dihydro-4H-pyrazolo[1,5-a]pyrazine-5-carboxylate (535 mg, 67% yield). LCMS ESI (+) m/z 657.1 (M+H).

Step E: Preparation of [7-(4-fluoro-2-isopropoxy-phenyl)-6-(4,5,6,7-tetrahydropyrazolo[1,5-a]pyrazin-2-yl)thieno[3,2-c]pyridin-4-yl]trifluoromethanesulfonate: To a solution of tert-butyl 2-[7-(4-fluoro-2-isopropoxy-phenyl)-4-(trifluoromethylsulfonyloxy)thieno[3,2-c]pyridin-6-yl]-6,7-dihydro-4H-pyrazolo[1,5-a]pyrazine-5-carboxylate (150 mg, 0.228 mmol) in DCM (6 mL) was added trifluoroacetic acid (1.5 mL) at 20° C. The mixture was stirred at 20° C. for 30 min. The mixture was concentrated to dryness to give crude [7-(4-fluoro-2-isopropoxy-phenyl)-6-(4,5,6,7-tetrahydropyrazolo[1,5-a]pyrazin-2-yl)thieno[3,2-c]pyridin-4-yl]trifluoromethanesulfonate (150 mg) which was used in the next step directly.

Step F: Preparation [7-(4-fluoro-2-isopropoxy-phenyl)-6-(5-prop-2-enoyl-6,7-dihydro-4H-pyrazolo[1,5-a]pyrazin-2-yl)thieno[3,2-c]pyridin-4-yl]trifluoromethanesulfonate: To a stirred solution of [7-(4-fluoro-2-isopropoxy-phenyl)-6-(4,5,6,7-tetrahydropyrazolo[1,5-a]pyrazin-2-yl)thieno[3,2-c]pyridin-4-yl]trifluoromethanesulfonate (127 mg, 0.228 mmol) and DIPEA (0.160 mL, 0.914 mmol) in DCM (6 mL) was added acryloyl chloride (0.020 mL, 0.25 mmol) at 0° C. The mixture was stirred at 0° C. for 30 min. NaHCO₃ aqueous solution was added to quench the reaction, and the product was extracted with DCM. The organic phase was dried over Na₂SO₄, filtered and concentrated to dryness to give crude product [7-(4-fluoro-2-isopropoxy-phenyl)-6-(5-prop-2-enoyl-6,7-dihydro-4H-pyrazolo[1,5-a]pyrazin-2-yl)thieno[3,2-c]pyridin-4-yl]trifluoromethanesulfonate (150 mg) which was used in the next step without further purification.

Step G: Preparation of 6-[7-(4-fluoro-2-isopropoxy-phenyl)-6-(5-prop-2-enoyl-6,7-dihydro-4H-pyrazolo[1,5-a]pyrazin-2-yl)thieno[3,2-c]pyridin-4-yl]-3,4-dihydro-2H-isoquinolin-1-one: A mixture of [7-(4-fluoro-2-isopropoxy-phenyl)-6-(5-prop-2-enoyl-6,7-dihydro-4H-pyrazolo[1,5-a]pyrazin-2-yl)thieno[3,2-c]pyridin-4-yl]trifluoromethanesulfonate (50 mg, 0.082 mmol), 6-(4,4,5,5-tetramethyl-1,3,2-dioxaborolan-2-yl)-3,4-dihydro-2H-isoquinolin-1-one (45 mg, 0.16 mmol), tetrakis(triphenyl phosphine)palladium(0) (10 mg, 0.0087 mmol) and sodium carbonate (17 mg, 0.16 mmol) in 1,4-dioxane (2 mL)/water (0.2 mL) was stirred at 95° C. for 2 hours under N₂. The mixture was concentrated and purified by reverse phase preparative HPLC to give 6-[7-(4-fluoro-2-isopropoxy-phenyl)-6-(5-prop-2-enoyl-6,7-dihydro-4H-pyrazolo[1,5-a]pyrazin-2-yl)thieno[3,2-c]pyridin-4-yl]-3,4-dihydro-2H-isoquinolin-1-one (33 mg, 65% yield) as a bis trifluoroacetic acid salt. LCMS ESI (+) m/z 608.3 (M+H). ¹H NMR (400 MHz, CD₃OD) δ 8.23 (d, J=7.9 Hz, 1H), 8.07 (d, J=5.6 Hz, 1H), 7.98 (d, J=10.5 Hz, 2H), 7.82 (d, J=5.7 Hz, 1H), 7.41 (d, J=7.4 Hz, 1H), 7.01 (dd, J=11.4, 2.1 Hz, 1H), 6.73-6.92 (m, 2H), 6.27 (d, J=16.6 Hz, 1H), 5.81 (s, 2H), 4.81 (m, 2H), 4.53-4.59 (m, 1H), 4.30 (s, 2H), 4.13 (t, J=5.7 Hz, 2H), 3.61 (t, J=6.6 Hz, 2H), 3.18 (t, J=6.5 Hz, 2H), 1.10 (d, J=6.0 Hz, 3H), 0.91 (d, J=6.0 Hz, 3H).

### Synthetic Example 37: Synthesis of 1-[2-[7-(4-fluoro-2-isopropoxy-phenyl)-4-(1-methylpyrazol-4-yl)thieno[3,2-c]pyridin-6-yl]-6,7-dihydro-4H-pyrazolo[1,5-a]pyrazin-5-yl]prop-2-en-1-one (Compound 200)

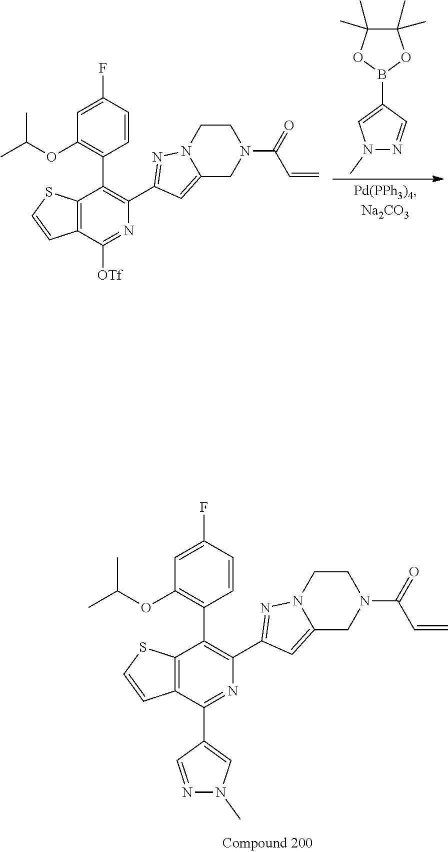

A mixture of [7-(4-fluoro-2-isopropoxy-phenyl)-6-(5-prop-2-enoyl-6,7-dihydro-4H-pyrazolo[1,5-a]pyrazin-2-yl)thieno[3,2-c]pyridin-4-yl]trifluoromethanesulfonate (50 mg, 0.082 mmol), 1-methyl-4-(4,4,5,5-tetramethyl-1,3,2-dioxaborolan-2-yl)pyrazole (34 mg, 0.16 mmol), tetrakis(triphenyl phosphine)palladium(0) (9.5 mg, 0.0082 mmol) and sodium carbonate (17 mg, 0.16 mmol) in 1,4-dioxane (2 mL)/water (0.2 mL) was stirred at 95° C. under N₂ for 2 hours. The mixture was filtered and concentrated. The residue was purified by reverse phase preparative HPLC to give 1-[2-[7-(4-fluoro-2-isopropoxy-phenyl)-4-(1-methylpyrazol-4-yl)thieno[3,2-c]pyridin-6-yl]-6,7-dihydro-4H-pyrazolo[1,5-a]pyrazin-5-yl]prop-2-en-1-one (38 mg, 85% yield) as a bis trifluoroacetic acid salt. LCMS ESI (+) m/z 543.3 (M+H). ¹H NMR (400 MHz, CD₃OD) δ 8.68 (s, 1H), 8.35 (s, 1H), 8.17 (d, J=5.8 Hz, 1H), 8.09 (d, J=5.6 Hz, 1H), 7.41 (t, J=7.5 Hz, 1H), 7.03 (dd, J=11.4, 2.3 Hz, 1H), 6.73-6.93 (m, 2H), 6.28 (d, J=16.9 Hz, 1H), 5.77-5.84 (m, 2H), 4.81 (m, 2H), 4.54-4.60 (m, 1H), 4.35 (s, 2H), 4.15 (t, J=5.7 Hz, 2H), 4.11 (s, 3H), 1.08 (d, J=6.0 Hz, 1H), 0.91 (d, J=6.0 Hz, 1H).

### Synthetic Example 38: Synthesis of 1-[2-[7-(4-fluoro-2-methoxy-phenyl)-4-(1-methylpyrazol-4-yl)thieno[3,2-c]pyridin-6-yl]-6,7-dihydro-4H-pyrazolo[1,5-a]pyrazin-5-yl]prop-2-en-1-one (Compound 206)

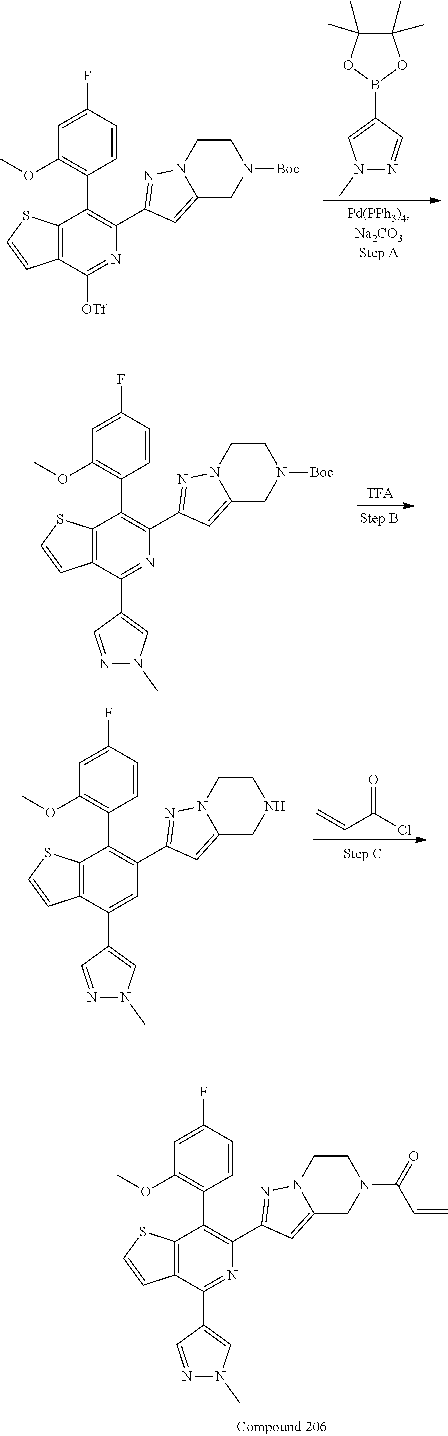

Step A: Preparation of tert-butyl 2-[7-(4-fluoro-2-methoxy-phenyl)-4-(1-methylpyrazol-4-yl)thieno[3,2-c]pyridin-6-yl]-6,7-dihydro-4H-pyrazolo[1,5-a]pyrazine-5-carboxylate: To a solution of tert-butyl 2-[7-(4-fluoro-2-methoxy-phenyl)-4-(trifluoromethylsulfonyloxy)thieno[3,2-c]pyridin-6-yl]-6,7-dihydro-4H-pyrazolo[1,5-a]pyrazine-5-carboxylate (160 mg, 0.255 mmol), 1-methyl-4-(4,4,5,5-tetramethyl-1,3,2-dioxaborolan-2-yl)pyrazole (79 mg, 0.38 mmol) and sodium carbonate (54 mg, 0.51 mmol) in 1,4-dioxane (2 mL) and water (0.2 mL) was added tetrakis(triphenylphosphine)palladium(0) (29 mg, 0.026 mmol). The mixture was stirred at 100° C. for 2 h under Ar. The mixture was poured into water. The product was extracted with EtOAc, dried over anhydrous Na₂SO₄, concentrated and purified by preparative TLC (DCM/MeOH: 20/1) to give tert-butyl 2-[7-(4-fluoro-2-methoxy-phenyl)-4-(1-methylpyrazol-4-yl)thieno[3,2-c]pyridin-6-yl]-6,7-dihydro-4H-pyrazolo[1,5-a]pyrazine-5-carboxylate (138 mg, 97% yield). LCMS ESI (+) m/z 561.2 (M+H).

Step B: Preparation of 7-(4-fluoro-2-methoxy-phenyl)-4-(1-methylpyrazol-4-yl)-6-(4,5,6,7-tetrahydropyrazolo[1,5-a]pyrazin-2-yl)thieno[3,2-c]pyridine: To a solution of tert-butyl 2-[7-(4-fluoro-2-methoxy-phenyl)-4-(1-methylpyrazol-4-yl)thieno[3,2-c]pyridin-6-yl]-6,7-dihydro-4H-pyrazolo[1,5-a]pyrazine-5-carboxylate (38 mg, 0.068 mmol) in DCM (2 mL) was added trifluoroacetic acid (0.60 mL). The mixture was stirred at 25° C. for 1.5 h. The mixture was concentrated to give crude 7-(4-fluoro-2-methoxy-phenyl)-4-(1-methylpyrazol-4-yl)-6-(4,5,6,7-tetrahydropyrazolo[1,5-a]pyrazin-2-yl)thieno[3,2-c]pyridine (48 mg) which was used in the next step directly. LCMS ESI (+) m/z 461.2 (M+H).

Step C: Preparation of 1-[2-[7-(4-fluoro-2-methoxy-phenyl)-4-(1-methylpyrazol-4-yl)thieno[3,2-c]pyridin-6-yl]-6,7-dihydro-4H-pyrazolo[1,5-a]pyrazin-5-yl]prop-2-en-1-one: To a solution of 7-(4-fluoro-2-methoxy-phenyl)-4-(1-methylpyrazol-4-yl)-6-(4,5,6,7-tetrahydropyrazolo[1,5-a]pyrazin-2-yl)thieno[3,2-c]pyridine (30 mg, 0.022 mmol) in DCM (1 vmL) was added triethylamine (0.0089 mL, 0.065 mmol) and acryloyl chloride (0.0018 mL, 0.022 mmol) at −60° C. The mixture was stirred at −60° C. for 1.5 h. The mixture was poured into NaHCO₃ aqueous solution and the product was extracted with EtOAc. The organic layers were washed with brine, dried over anhydrous Na₂SO₄ and concentrated. The residue was purified by reverse phase preparative HPLC to give 1-[2-[7-(4-fluoro-2-methoxy-phenyl)-4-(1-methylpyrazol-4-yl)thieno[3,2-c]pyridin-6-yl]-6,7-dihydro-4H-pyrazolo[1,5-a]pyrazin-5-yl]prop-2-en-1-one (16 mg, 49% yield) as a trifluoroacetic acid salt. LCMS ESI (+) m/z 515.2 (M+H). ¹H NMR (400 MHz, CD₃OD) δ 8.67 (s, 1H), 8.34 (s, 1H), 8.15 (d, J=5.6 Hz, 1H), 8.08 (d, J=5.6 Hz, 1H), 7.39 (dd, J=8.2, 6.7 Hz, 1H), 7.07 (dd, J=10.9, 2.2 Hz, 1H), 6.72-6.96 (m, 2H), 6.28 (d, J=16.6 Hz, 1H), 5.77-5.83 (m, 2H), 4.86-4.89 (m, 1H), 4.77 (s, 1H), 4.35 (s, 2H), 4.16 (t, J=5.7 Hz, 2H), 4.11 (s, 3H), 3.67 (s, 3H).

### Synthetic Example 39: Synthesis of 6-[7-(4-fluoro-2-isopropoxy-phenyl)-6-[5-(2-fluoroprop-2-enoyl)-6,7-dihydro-4H-pyrazolo[1,5-a]pyrazin-2-yl]thieno[3,2-c]pyridin-4-yl]-3,4-dihydro-2H-isoquinolin-1-one (Compound 258)

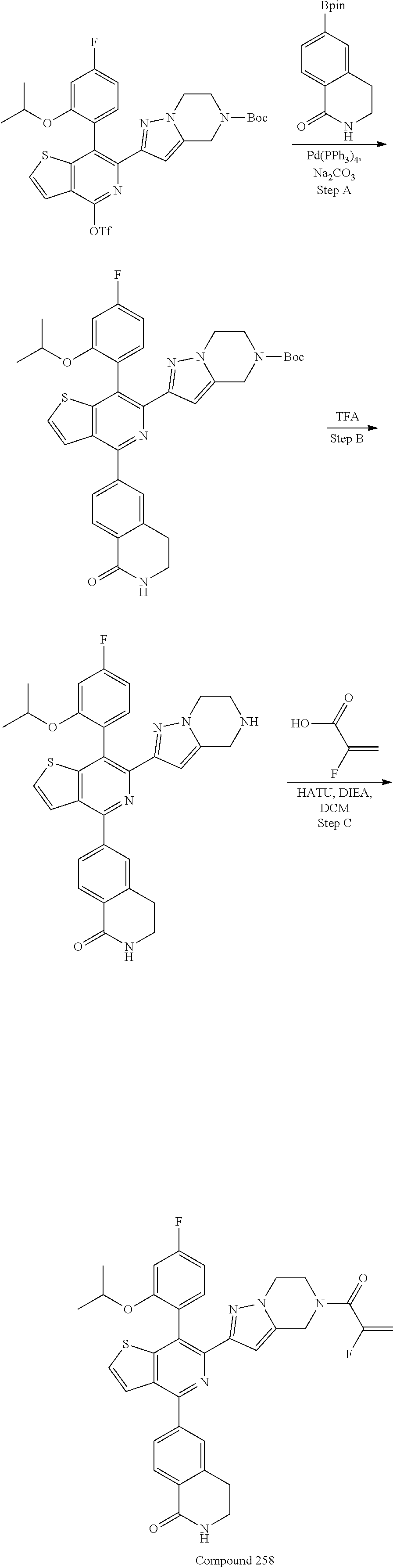

Step A: Preparation of tert-butyl 2-[7-(4-fluoro-2-isopropoxy-phenyl)-4-(1-oxo-3,4-dihydro-2H-isoquinolin-6-yl)thieno[3,2-c]pyridin-6-yl]-6,7-dihydro-4H-pyrazolo[1,5-a]pyrazine-5-carboxylate: A mixture of tert-butyl 2-[7-(4-fluoro-2-isopropoxy-phenyl)-4-(trifluoromethylsulfonyloxy)thieno[3,2-c]pyridin-6-yl]-6,7-dihydro-4H-pyrazolo[1,5-a]pyrazine-5-carboxylate (120 mg, 0.183 mmol), 6-(4,4,5,5-tetramethyl-1,3,2-dioxaborolan-2-yl)-3,4-dihydro-2H-isoquinolin-1-one (100 mg, 0.365 mmol), tetrakis(triphenylphosphine)palladium(0) (21 mg, 0.018 mmol) and sodium carbonate (39 mg, 0.37 mmol) in 1,4-dioxane (10 mL)/water (1 mL) was stirred at 85° C. under N₂ for 2 hours. The mixture was poured into water. The reaction was extracted with DCM, dried over anhydrous Na₂SO₄, concentrated and purified by preparative TLC (DCM:MeOH=10:1) to give tert-butyl 2-[7-(4-fluoro-2-isopropoxy-phenyl)-4-(1-oxo-3,4-dihydro-2H-isoquinolin-6-yl)thieno[3,2-c]pyridin-6-yl]-6,7-dihydro-4H-pyrazolo[1,5-a]pyrazine-5-carboxylate (120 mg, 99% yield). LCMS ESI (+) m/z 654.4 (M+H).

Step B: Preparation of 6-[7-(4-fluoro-2-isopropoxy-phenyl)-6-(4,5,6,7-tetrahydropyrazolo[1,5-a]pyrazin-2-yl)thieno[3,2-c]pyridin-4-yl]-3,4-dihydro-2H-isoquinolin-1-one: To a solution of tert-butyl 2-[7-(4-fluoro-2-isopropoxy-phenyl)-4-(1-oxo-3,4-dihydro-2H-isoquinolin-6-yl)thieno[3,2-c]pyridin-6-yl]-6,7-dihydro-4H-pyrazolo[1,5-a]pyrazine-5-carboxylate (120 mg, 0.181 mmol) in DCM (4 mL) was added trifluoroacetic acid (1.5 mL). The mixture was stirred at 25° C. for 1 hour. The mixture was concentrated to give 6-[7-(4-fluoro-2-isopropoxy-phenyl)-6-(4,5,6,7-tetrahydropyrazolo[1,5-a]pyrazin-2-yl)thieno[3,2-c]pyridin-4-yl]-3,4-dihydro-2H-isoquinolin-1-one (150 mg, crude), which was used in the next step without further purification. LCMS ESI (+) m/z 554.3 (M+H).

Step C: Preparation of 6-[7-(4-fluoro-2-isopropoxy-phenyl)-6-[5-(2-fluoroprop-2-enoyl)-6,7-dihydro-4H-pyrazolo[1,5-a]pyrazin-2-yl]thieno[3,2-c]pyridin-4-yl]-3,4-dihydro-2H-isoquinolin-1-one: To a solution of 2-fluoroprop-2-enoic acid (6.1 mg, 0.068 mmol) and HATU (26 mg, 0.068 mmol) in DCM (3 mL) was added DIEA (0.040 mL, 0.23 mmol) followed by 6-[7-(4-fluoro-2-isopropoxy-phenyl)-6-(4,5,6,7-tetrahydropyrazolo[1,5-a]pyrazin-2-yl)thieno[3,2-c]pyridin-4-yl]-3,4-dihydro-2H-isoquinolin-1-one (25 mg, 0.045 mmol). The mixture was stirred at 25° C. for 40 mins. The mixture was poured into NaHCO₃ aqueous solution, and the product was extracted with EtOAc. The organic phase was dried over anhydrous Na₂SO₄, concentrated and purified by reverse phase preparative HPLC to give 6-[7-(4-fluoro-2-isopropoxy-phenyl)-6-[5-(2-fluoroprop-2-enoyl)-6,7-dihydro-4H-pyrazolo[1,5-a]pyrazin-2-yl]thieno[3,2-c]pyridin-4-yl]-3,4-dihydro-2H-isoquinolin-1-one (13 mg, 46% yield) as a bis trifluoroacetic acid salt. LCMS ESI (+) m/z 626.2 (M+H). ¹H NMR (400 MHz, CD₃OD) δ 8.24 (d, J=7.9 Hz, 1H), 8.08 (d, J=5.6 Hz, 1H), 7.97-8.01 (m, 2H), 7.82 (d, J=5.6 Hz, 1H), 7.41 (t, J=7.7 Hz, 1H), 7.00 (d=10.1 Hz, 1H), 6.89 (t, J=6.4 Hz, 1H), 5.84 (s, 1H), 5.28-5.41 (m, 2H), 4.77 (s, 2H), 4.54-4.60 (m, 1H), 4.33 (t, J=5.6 Hz, 2H), 4.01 (t, J=5.2 Hz, 2H), 3.62 (t, J=6.6 Hz, 2H), 3.19 (t, J=6.5 Hz, 2H), 1.10 (d, J=6.0 Hz, 3H), 0.93 (d, J=6.0 Hz, 3H).

### Synthetic Example 40: Synthesis of 5-[5-(4-fluoro-2-isopropoxy-phenyl)-6-(5-prop-2-enoyl-6,7-dihydro-4H-pyrazolo[1,5-a]pyrazin-2-yl)pyrazin-2-yl]-1-methyl-pyridin-2-one (Compound 273)

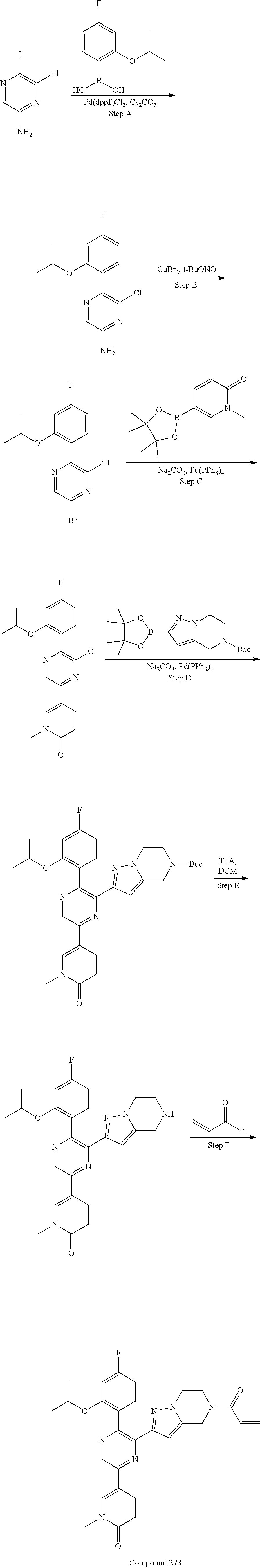

Step A: Preparation of 6-chloro-5-(4-fluoro-2-isopropoxy-phenyl)pyrazin-2-amine: To a solution of 6-chloro-5-iodo-pyrazin-2-amine (900 mg, 3.52 mmol) and (4-fluoro-2-isopropoxy-phenyl) boronic acid (838 mg, 4.20 mmol) in a mixture of 1,4-dioxane (10 mL) and 2 M Cs₂CO₃ aqueous solution (1 mL) was added Pd(dppf)Cl₂(128 mg, 0.176 mmol). The mixture was stirred at 90° C. for 4 h under Ar. The reaction mixture was diluted with water and extracted with EtOAc. The combined organic layers were dried over sodium sulfate, filtered and concentrated under reduced pressure. The residue was purified by column chromatography (EtOAc:petroleum ether=1:3) to give 6-chloro-5-(4-fluoro-2-isopropoxy-phenyl)pyrazin-2-amine (900 mg, 91% yield) as a yellow solid. LCMS ESI (+) m/z 282.2 (M+H).

Step B: Preparation of 5-bromo-3-chloro-2-(4-fluoro-2-isopropoxy-phenyl)pyrazine: To a solution of 6-chloro-5-(4-fluoro-2-isopropoxy-phenyl)pyrazin-2-amine (300 mg, 1.06 mmol) and cupric bromide (356 mg, 1.60 mmol) in MeCN (10 ml) was added tert-butyl nitrite (165 mg, 1.60 mmol). The mixture was stirred at 25° C. for 1 hour. The reaction mixture was diluted with water and extracted with EtOAc twice. The combined organic layers were washed with brine, dried over Na₂SO₄ and concentrated. The residue was purified by column chromatography (EtOAc:petroleum ether=1:5) to give 5-bromo-3-chloro-2-(4-fluoro-2-isopropoxy-phenyl)pyrazine (100 mg, 27% yield) as a brown solid. LCMS ESI (+) m/z 345.1 (M+H).

Step C: Preparation of 5-[6-chloro-5-(4-fluoro-2-isopropoxy-phenyl)pyrazin-2-yl]-1-methyl-pyridin-2-one: To a solution of 5-bromo-3-chloro-2-(4-fluoro-2-isopropoxy-phenyl)pyrazine (100 mg, 0.289 mmol) and 1-methyl-5-(4,4,5,5-tetramethyl-1,3,2-dioxaborolan-2-yl)pyridin-2-one (82 mg, 0.35 mmol) in dioxane (4 ml) and water (0.4 ml) were added Na₂CO₃ (61 mg, 0.58 mmol) and tetrakis(triphenylphosphine)palladium (33 mg, 0.029 mmol). The reaction mixture was stirred at 60° C. for 1 h under Ar. The reaction mixture was diluted with water and extracted with EtOAc. The organic layer was evaporated and the resulting residue was purified by preparative TLC (EtOAc:petroleum ether=1:1) to give 5-[6-chloro-5-(4-fluoro-2-isopropoxy-phenyl)pyrazin-2-yl]-1-methyl-pyridin-2-one (30 mg, 35% yield) as a yellow solid. LCMS ESI (+) m/z 374.1 (M+H).

Step D: Preparation of tert-butyl 2-[3-(4-fluoro-2-isopropoxy-phenyl)-6-(1-methyl-6-oxo-3-pyridyl)pyrazin-2-yl]-6,7-dihydro-4H-pyrazolo[1,5-a]pyrazine-5-carboxylate: To a solution of 5-[6-chloro-5-(4-fluoro-2-isopropoxy-phenyl)pyrazin-2-yl]-1-methyl-pyridin-2-one (30 mg, 0.080 mmol) and tert-butyl 2-(4,4,5,5-tetramethyl-1,3,2-dioxaborolan-2-yl)-6,7-dihydro-4H-pyrazolo[1,5-a]pyrazine-5-carboxylate (34 mg, 0.096 mmol) in 1,4-dioxane (2 ml) and water (0.6 ml) were added Na₂CO₃ (17 mg, 0.16 mmol) and tetrakis(triphenylphosphine)palladium (9.3 mg, 0.0080 mmol). The reaction mixture was stirred at 90° C. for 1 h under Ar. The reaction mixture was diluted with water and extracted with EtOAc. The organic layer was evaporated and the residue was purified by preparative TLC (DCM:MeOH=10:1) to give tert-butyl 2-[3-(4-fluoro-2-isopropoxy-phenyl)-6-(1-methyl-6-oxo-3-pyridyl)pyrazin-2-yl]-6,7-dihydro-4H-pyrazolo[1,5-a]pyrazine-5-carboxylate (20 mg, 67% yield) as a yellow solid. LCMS ESI (+) m/z 561.3 (M+H).

Step E: Preparation of 5-[5-(4-fluoro-2-isopropoxy-phenyl)-6-(4,5,6,7-tetrahydropyrazolo[1,5-a]pyrazin-2-yl)pyrazin-2-yl]-1-methyl-pyridin-2-one: To a solution of tert-butyl 2-[3-(4-fluoro-2-isopropoxy-phenyl)-6-(1-methyl-6-oxo-3-pyridyl)pyrazin-2-yl]-6,7-dihydro-4H-pyrazolo[1,5-a]pyrazine-5-carboxylate (20 mg, 0.036 mmol) in DCM (1 mL) was added trifluoroacetic acid (1 mL). The mixture was stirred at 25° C. for 2 h. The mixture was concentrated to dryness to give crude 5-[5-(4-fluoro-2-isopropoxy-phenyl)-6-(4,5,6,7-tetrahydropyrazolo[1,5-a]pyrazin-2-yl)pyrazin-2-yl]-1-methyl-pyridin-2-one (30 mg) which was used in the next step directly. LCMS ESI (+) m/z 461.2 (M+H).

Step F: Preparation of 5-[5-(4-fluoro-2-isopropoxy-phenyl)-6-(5-prop-2-enoyl-6,7-dihydro-4H-pyrazolo[1,5-a]pyrazin-2-yl)pyrazin-2-yl]-1-methyl-pyridin-2-one: To a solution of 5-[5-(4-fluoro-2-isopropoxy-phenyl)-6-(4,5,6,7-tetrahydropyrazolo[1,5-a]pyrazin-2-yl)pyrazin-2-yl]-1-methyl-pyridin-2-one (30 mg) in DCM (2 mL) were added triethylamine (0.060 mL, 0.43 mmol) and acryloyl chloride (0.010 mL, 0.13 mmol) at −60° C. The mixture was stirred at −60° C. for 1 h. The mixture was poured into NaHCO₃ aqueous solution and the product was extracted with EtOAc. The organic phase was dried over anhydrous Na₂SO₄, concentrated and purified by reverse phase preparative HPLC to give 5-[5-(4-fluoro-2-isopropoxy-phenyl)-6-(5-prop-2-enoyl-6,7-dihydro-4H-pyrazolo[1,5-a]pyrazin-2-yl)pyrazin-2-yl]-1-methyl-pyridin-2-one (10 mg, 43% yield) as a trifluoroacetic acid salt. LCMS ESI (+) m/z 515.3 (M+H). ¹H NMR (400 MHz, CD₃OD) δ 8.94 (s, 1H), 8.78-8.81 (m, 1H), 8.40-8.45 (m, 1H), 7.42-7.48 (m, 1H), 6.66-6.90 (m, 4H), 6.28 (d, J=16.8 Hz, 1H), 5.99 (s, 1H), 5.82 (d, J=9.9 Hz, 1H), 4.77 (s, 1H), 4.34-4.44 (m, 1H), 4.17-4.26 (m, 2H), 4.08-4.16 (m, 2H), 3.71 (s, 3H), 0.73-1.14 (m, 6H).

### Synthetic Example 41: Synthesis of 1-[2-[3-fluoro-7-(4-fluoro-2-methoxy-phenyl)-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (Compound 274)

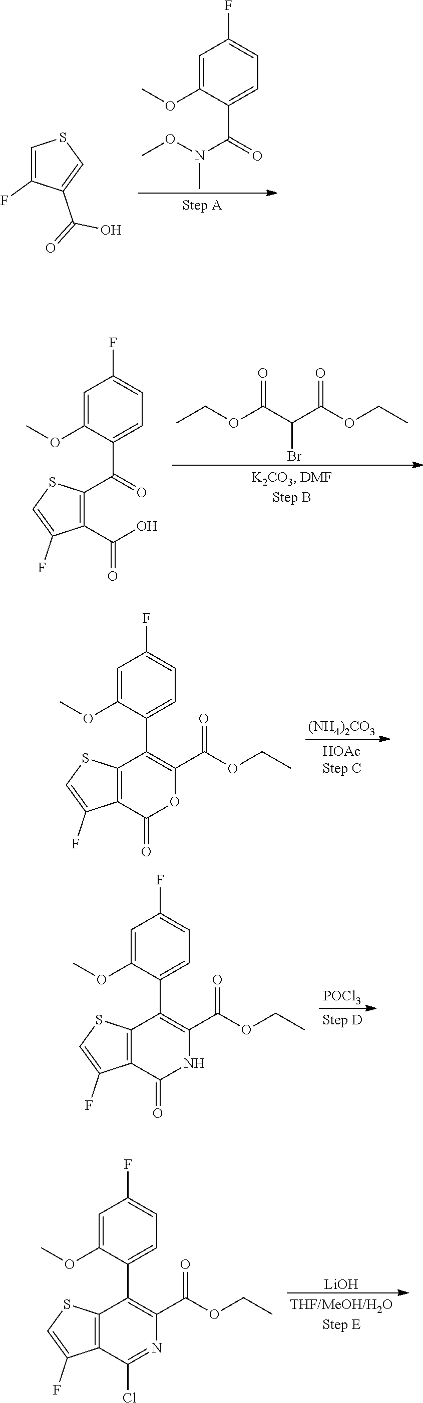

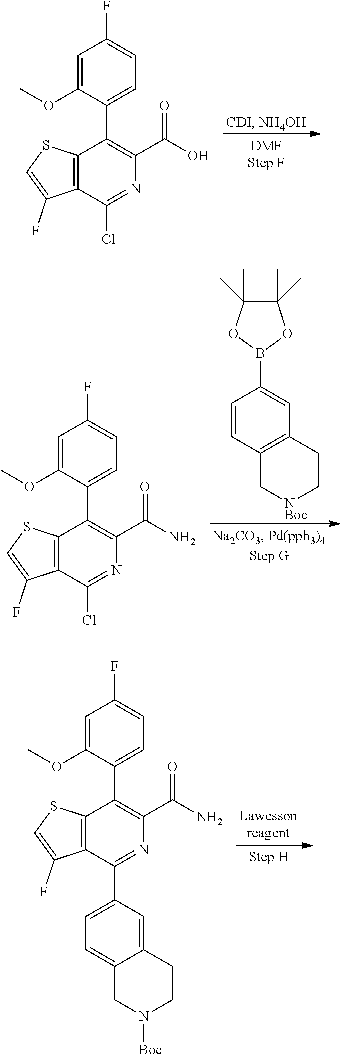

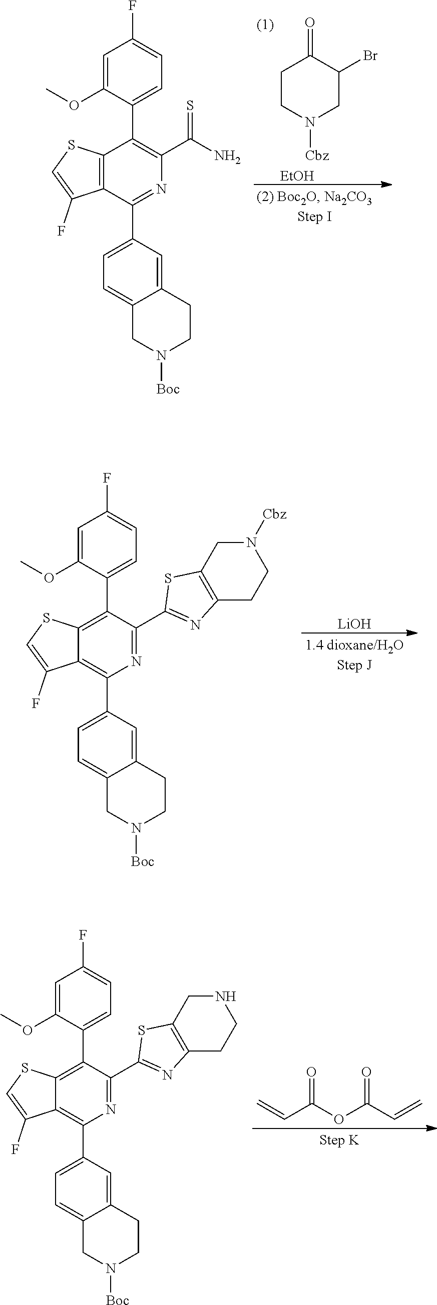

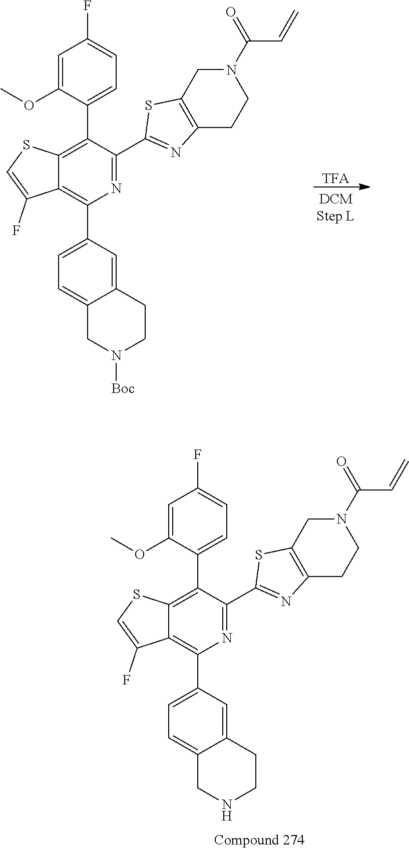

Step A: Preparation of 4-fluoro-2-(4-fluoro-2-methoxy-benzoyl)thiophene-3-carboxylic acid: To a solution of 4-fluorothiophene-3-carboxylic acid (200 mg, 1.37 mmol) in THF (5 mL) was added LiHMDS (2.9 mL, 2.9 mmol) at −45° C. The mixture was stirred at −45° C. for 0.5 h. 4-Fluoro-N,2-dimethoxy-N-methyl-benzamide (379 mg, 1.78 mmol) was added. The mixture was stirred at ambient temperature for 1 h. 1N HCl (10 mL) was added to quench the reaction. The mixture was poured into water (30 mL) and extracted with EtOAc (50 mL). The combined organic layers were washed with brine, dried over Na₂SO₄ and concentrated. The residue was purified by preparative HPLC to give 4-fluoro-2-(4-fluoro-2-methoxy-benzoyl)thiophene-3-carboxylic acid (300 mg, 74% yield). LCMS ESI (+) m/z 299 (M+H).

Step B: Preparation of ethyl 3-fluoro-7-(4-fluoro-2-methoxy-phenyl)-4-oxo-thieno[3,2-c]pyran-6-carboxylate: To a solution of 4-fluoro-2-(4-fluoro-2-methoxy-benzoyl)thiophene-3-carboxylic acid (100 mg, 0.335 mmol) in DMF (5 mL) were added potassium carbonate (51 mg, 0.37 mmol) and diethyl 2-bromopropanedioate (64 mg, 0.27 mmol). The mixture was stirred at 30° C. for 5 days. The mixture was poured into water (30 mL) and extracted with EtOAc (50 mL×2). The combined organic layers were washed with brine, dried over Na₂SO₄ and concentrated. The residue was purified by preparative TLC (petroleum ether:EtOAc=5:1) to give ethyl 3-fluoro-7-(4-fluoro-2-methoxy-phenyl)-4-oxo-thieno[3,2-c]pyran-6-carboxylate (70 mg, 57% yield). LCMS ESI (+) m/z 367.1 (M+H).

Step C: Preparation of ethyl 3-fluoro-7-(4-fluoro-2-methoxy-phenyl)-4-oxo-5H-thieno[3,2-c]pyridine-6-carboxylate: To a solution of ethyl 3-fluoro-7-(4-fluoro-2-methoxy-phenyl)-4-oxo-thieno[3,2-c]pyran-6-carboxylate (300 mg, 0.819 mmol) in acetic acid (5 mL) were added ammonium carbonate (786 mg, 8.19 mmol). The mixture was stirred at 90° C. for 48 h. The mixture was poured into water and extracted with EtAOc. The combined organic layers were washed with brine, dried over Na₂SO₄ and concentrated. The residue was purified by preparative TLC (petroleum ether:EtOAc 2:1) to give ethyl 3-fluoro-7-(4-fluoro-2-methoxy-phenyl)-4-oxo-5H-thieno[3,2-c]pyridine-6-carboxylate (110 mg, 37% yield). LCMS ESI (+) m/z 366.1 (M+H).

Step D: Preparation of ethyl 4-chloro-3-fluoro-7-(4-fluoro-2-methoxy-phenyl)thieno[3,2-c]pyridine-6-carboxylate: A solution of ethyl 3-fluoro-7-(4-fluoro-2-methoxy-phenyl)-4-oxo-5H-thieno[3,2-c]pyridine-6-carboxylate (50 mg, 0.14 mmol) in phosphorus oxychloride (3.00 mL, 32.2 mmol) was stirred at 90° C. for 2 h. The mixture was poured into NaHCO₃ aqueous solution and extracted with EtOAc. The organic layer was washed with brine, dried over Na₂SO₄ and concentrated. The residue was purified by preparative TLC (petroleum ether:EtOAc 4:1) to give ethyl 4-chloro-3-fluoro-7-(4-fluoro-2-methoxy-phenyl)thieno[3,2-c]pyridine-6-carboxylate (35 mg, 67% yield). LCMS ESI (+) m/z 384 (M+H).

Step E: Preparation of 4-chloro-3-fluoro-7-(4-fluoro-2-methoxy-phenyl)thieno[3,2-c]pyridine-6-carboxylic acid: To a solution of ethyl 4-chloro-3-fluoro-7-(4-fluoro-2-methoxy-phenyl)thieno[3,2-c]pyridine-6-carboxylate (50 mg, 0.13 mmol) in THF (3 mL), methanol (1 mL) and water (1 mL) was added lithium hydroxide monohydrate (27 mg, 0.65 mmol). The mixture was stirred at 25° C. for 3 h. The mixture was poured into water. 1 N HCl was added to adjust the pH to 3. The mixture was extracted with EtOAc. The combined organic layers were washed with brine, dried over Na₂SO₄ and concentrated to give crude 4-chloro-3-fluoro-7-(4-fluoro-2-methoxy-phenyl)thieno[3,2-c]pyridine-6-carboxylic acid (47 mg, 100% yield), which was used in the next step directly. LCMS ESI (+) m/z 356 (M+H).

Step F: Preparation of 4-chloro-3-fluoro-7-(4-fluoro-2-methoxy-phenyl)thieno[3,2-c]pyridine-6-carboxamide: To a solution of 4-chloro-3-fluoro-7-(4-fluoro-2-methoxy-phenyl)thieno[3,2-c]pyridine-6-carboxylic acid (120 mg, 0.337 mmol) in DMF (4 mL) was added CDI (82 mg, 0.51 mmol). The mixture was stirred at 25° C. for 0.5 h. The mixture was poured into NH₄OH (5.0 mL). The resulting mixture was stirred at 25° C. for 0.5 h. The mixture was poured into water and extracted with EtOAc. The combined organic layers were washed with brine, dried over Na₂SO₄ and concentrated. The residue was purified by preparative TLC (petroleum ether:EtOAc 3:1) to give 4-chloro-3-fluoro-7-(4-fluoro-2-methoxy-phenyl)thieno[3,2-c]pyridine-6-carboxamide (100 mg, 84% yield). LCMS ESI (+) m/z 355 (M+H).

Step G: Preparation of tert-butyl 6-[6-carbamoyl-3-fluoro-7-(4-fluoro-2-methoxy-phenyl)thieno[3,2-c]pyridin-4-yl]-3,4-dihydro-1H-isoquinoline-2-carboxylate: To a solution of 4-chloro-3-fluoro-7-(4-fluoro-2-methoxy-phenyl)thieno[3,2-c]pyridine-6-carboxamide (135 mg, 0.381 mmol) in 1,4-dioxane (5 mL) and water (0.5 mL) were added tert-butyl 6-(4,4,5,5-tetramethyl-1,3,2-dioxaborolan-2-yl)-3,4-dihydro-1H-isoquinoline-2-carboxylate (178 mg, 0.496 mmol), sodium carbonate (101 mg, 0.953 mmol) and tetrakis(triphenylphosphine)palladium(0) (66 mg, 0.057 mmol). The mixture was stirred at 90° C. for 4 h. The mixture was concentrated and purified by preparative TLC (petroleum ether:EtOAc 2:1) to give tert-butyl 6-[6-carbamoyl-3-fluoro-7-(4-fluoro-2-methoxy-phenyl)thieno[3,2-c]pyridin-4-yl]-3,4-dihydro-1H-isoquinoline-2-carboxylate (107 mg, 51% yield). LCMS ESI (+) m/z 552.2 (M+H).

Step H: Preparation of tert-butyl 6-[6-carbamothioyl-3-fluoro-7-(4-fluoro-2-methoxy-phenyl)thieno[3,2-c]pyridin-4-yl]-3,4-dihydro-1H-isoquinoline-2-carboxylate: To a solution of tert-butyl 6-[6-carbamoyl-3-fluoro-7-(4-fluoro-2-methoxy-phenyl)thieno[3,2-c]pyridin-4-yl]-3,4-dihydro-1H-isoquinoline-2-carboxylate (107 mg, 0.194 mmol) in THF (5 mL) was added Lawesson's reagent (118 mg, 0.291 mmol). The mixture was stirred at 70° C. for 12 h. The mixture was poured into water and extracted with EtOAc. The organic layer was washed with brine, dried over Na₂SO₄ and concentrated. The residue was purified by preparative TLC (petroleum ether:EtOAc 2:1) to give tert-butyl 6-[6-carbamothioyl-3-fluoro-7-(4-fluoro-2-methoxy-phenyl)thieno[3,2-c]pyridin-4-yl]-3,4-dihydro-1H-isoquinoline-2-carboxylate (55 mg, 50% yield). LCMS ESI (+) m/z 568.2 (M+H).

Step I: Preparation of benzyl 2-[4-(2-tert-butoxycarbonyl-3,4-dihydro-1H-isoquinolin-6-yl)-3-fluoro-7-(4-fluoro-2-methoxy-phenyl)thieno[3,2-c]pyridin-6-yl]-6,7-dihydro-4H-thiazolo[5,4-c]pyridine-5-carboxylate: To a solution of tert-butyl 6-[6-carbamothioyl-3-fluoro-7-(4-fluoro-2-methoxy-phenyl)thieno[3,2-c]pyridin-4-yl]-3,4-dihydro-1H-isoquinoline-2-carboxylate (55 mg, 0.097 mmol) in EtOH (3 mL) was added benzyl 3-bromo-4-oxo-piperidine-1-carboxylate (67 mg, 0.21 mmol). The mixture was stirred at 70° C. for 12 h. The mixture was diluted with water and THF. Na₂CO₃ (21 mg, 0.20 mmol) and Boc₂O (22 mg, 0.10 mmol) were added. The resulting mixture was stirred at ambient temperature for 0.5 h. The product was extracted with EtOAc. The combined organic layers were washed with brine, dried over Na₂SO₄ and concentrated. The residue was purified by preparative TLC (petroleum ether:EtOAc 1:1) to give benzyl 2-[4-(2-tert-butoxycarbonyl-3,4-dihydro-1H-isoquinolin-6-yl)-3-fluoro-7-(4-fluoro-2-methoxy-phenyl)thieno[3,2-c]pyridin-6-yl]-6,7-dihydro-4H-thiazolo[5,4-c]pyridine-5-carboxylate (39 mg, 52% yield). LCMS ESI (+) m/z 781 (M+H).

Step J: Preparation of tert-butyl 6-[3-fluoro-7-(4-fluoro-2-methoxy-phenyl)-6-(4,5,6,7-tetrahydrothiazolo[5,4-c]pyridin-2-yl)thieno[3,2-c]pyridin-4-yl]-3,4-dihydro-1H-isoquinoline-2-carboxylate: To a solution of benzyl 2-[4-(2-tert-butoxycarbonyl-3,4-dihydro-1H-isoquinolin-6-yl)-3-fluoro-7-(4-fluoro-2-methoxy-phenyl)thieno[3,2-c]pyridin-6-yl]-6,7-dihydro-4H-thiazolo[5,4-c]pyridine-5-carboxylate (25 mg, 0.032 mmol) in 1,4-dioxane (1 mL) and water (1 mL) was added lithium hydroxide (100 mg, 2.4 mmol). The mixture was stirred at 100° C. for 12 h. The mixture was poured into water and extracted with EtOAc. The organic layer was washed with brine, dried over Na₂SO₄ and concentrated to give crude tert-butyl 6-[3-fluoro-7-(4-fluoro-2-methoxy-phenyl)-6-(4,5,6,7-tetrahydrothiazolo[5,4-c]pyridin-2-yl)thieno[3,2-c]pyridin-4-yl]-3,4-dihydro-1H-isoquinoline-2-carboxylate (18 mg, 88% yield), which was used in the next step directly. LCMS ESI (+) m/z 647.2 (M+H).

Step K: Preparation of tert-butyl 6-[3-fluoro-7-(4-fluoro-2-methoxy-phenyl)-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate: To a solution of tert-butyl 6-[3-fluoro-7-(4-fluoro-2-methoxy-phenyl)-6-(4,5,6,7-tetrahydrothiazolo[5,4-c]pyridin-2-yl)thieno[3,2-c]pyridin-4-yl]-3,4-dihydro-1H-isoquinoline-2-carboxylate (18 mg, 0.028 mmol) in DCM (5 mL) were added triethylamine (0.012 mL, 0.084 mmol) and prop-2-enoyl prop-2-enoate (3.5 mg, 0.028 mmol) at −60° C. The mixture was stirred at 0° C. for 0.5 h. NaHCO₃ aqueous solution was added. The mixture was diluted with water and extracted with EtOAc. The combined organic layers were washed with brine, dried over Na₂SO₄ and concentrated. The residue was purified by preparative TLC (petroleum ether:EtOAc 1:1) to give tert-butyl 6-[3-fluoro-7-(4-fluoro-2-methoxy-phenyl)-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (22 mg, 90% yield). LCMS ESI (+) m/z 701 (M+H).

Step L: Preparation of 1-[2-[3-fluoro-7-(4-fluoro-2-methoxy-phenyl)-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one: To a solution of tert-butyl 6-[3-fluoro-7-(4-fluoro-2-methoxy-phenyl)-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (22 mg, 0.031 mmol) in DCM (4 mL) was added trifluoroacetic acid (1.0 mL). The mixture was stirred at 25° C. for 1 h. The mixture was concentrated and the residue was purified by reverse phase preparative HPLC to give 1-[2-[3-fluoro-7-(4-fluoro-2-methoxy-phenyl)-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (16 mg, 83% yield) as a bis trifluoroacetic acid salt. LCMS ESI (+) m/z 601.2 (M+H). ¹H NMR (400 MHz, CD₃OD) δ 7.70-7.71 (m, 2H), 7.37-7.41 (m, 2H), 7.30 (t, J=8.4 Hz, 1H), 6.93 (d, J=10.8 Hz, 1H), 6.74-6.88 (m, 2H), 6.22-6.26 (m, 1H), 5.76-5.80 (m, 1H), 4.84 (s, 2H), 4.49 (s, 2H), 3.90-3.91 (m, 2H), 3.58-3.60 (m, 5H), 3.25-3.26 (m, 2H), 2.70-2.71 (m, 2H).

### Synthetic Example 42: Synthesis of 1-[2-[3-(4-fluoro-2-isopropoxy-phenyl)-6-(1-methylpyrazol-4-yl)-2-pyridyl]-6,7-dihydro-4H-pyrazolo[1,5-a]pyrazin-5-yl]prop-2-en-1-one (Compound 279)

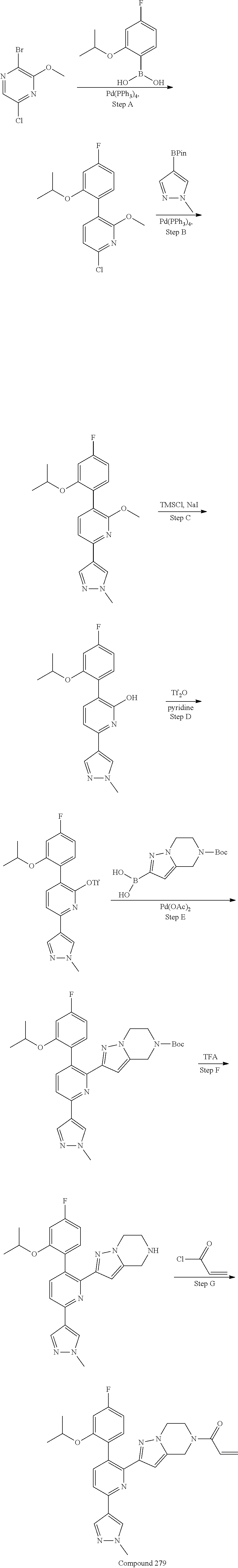

Step A: Preparation of 6-chloro-3-(4-fluoro-2-isopropoxy-phenyl)-2-methoxy-pyridine: To a solution of 3-bromo-6-chloro-2-methoxy-pyridine (400 mg, 1.80 mmol), (4-fluoro-2-isopropoxy-phenyl)boronic acid (712 mg, 3.60 mmol) and sodium carbonate (381 mg, 3.60 mmol) in 1,4-dioxane (1 mL) and water (0.1 mL) was added Pd(PPh₃)₄ (208 mg, 0.180 mmol). The mixture was stirred at 80° C. for 5 h under Ar. The mixture was poured into water and the product was extracted with EtOAc. The combined organic layers were dried over anhydrous Na₂SO₄, concentrated and purified by preparative TLC (3% EtOAc in petroleum ether) to give 6-chloro-3-(4-fluoro-2-isopropoxy-phenyl)-2-methoxy-pyridine (106 mg, 20% yield). LCMS ESI (+) m/z 296.2 (M+H).

Step B: Preparation of 3-(4-fluoro-2-isopropoxy-phenyl)-2-methoxy-6-(1-methylpyrazol-4-yl)pyridine: To a solution of 6-chloro-3-(4-fluoro-2-isopropoxy-phenyl)-2-methoxy-pyridine (99 mg, 0.34 mmol), 1-methyl-4-(4,4,5,5-tetramethyl-1,3,2-dioxaborolan-2-yl)pyrazole (70 mg, 0.34 mmol) and sodium carbonate (71 mg, 0.67 mmol) in 1,4-dioxane (2 mL) and water (0.2 mL) was added tetrakis(triphenylphosphine)palladium (39 mg, 0.034 mmol). The mixture was stirred at 100° C. for 8 h under Ar. The mixture was concentrated and purified by preparative TLC (EtOAc/petroleum ether 1/3) to give 3-(4-fluoro-2-isopropoxy-phenyl)-2-methoxy-6-(1-methylpyrazol-4-yl)pyridine (112 mg, 98% yield). LCMS ESI (+) m/z 342.2 (M+H).

Step C: Preparation of 3-(4-fluoro-2-isopropoxy-phenyl)-6-(1-methylpyrazol-4-yl)pyridin-2-ol: To a solution of 3-(4-fluoro-2-isopropoxy-phenyl)-2-methoxy-6-(1-methylpyrazol-4-yl)pyridine (140 mg, 0.410 mmol) in MeCN (3 mL) were added sodium iodide (184 mg, 1.23 mmol) and chlorotrimethyl silane (134 mg, 1.23 mmol). The mixture was stirred at 25° C. for 8 h. The mixture was concentrated and purified by preparative TLC (DCM/MeOH 20/1) to give 3-(4-fluoro-2-isopropoxy-phenyl)-6-(1-methylpyrazol-4-yl)pyridin-2-ol (95 mg, 71% yield). LCMS ESI (+) m/z 328.1 (M+H).

Step D: Preparation of [3-(4-fluoro-2-isopropoxy-phenyl)-6-(1-methylpyrazol-4-yl)-2-pyridyl]trifluoromethanesulfonate: To a solution of 3-(4-fluoro-2-isopropoxy-phenyl)-6-(1-methylpyrazol-4-yl)pyridin-2-ol (70 mg, 0.21 mmol) and pyridine (169 mg, 2.14 mmol) in DCM (3 mL) was added trifluoromethanesulfonic anhydride (0.072 mL, 0.43 mmol). The mixture was stirred at 25° C. for 2 h. The mixture was concentrated and purified by preparative TLC (EtOAc/petroleum ether: 1/3) to give [3-(4-fluoro-2-isopropoxy-phenyl)-6-(1-methylpyrazol-4-yl)-2-pyridyl]trifluoromethanesulfonate (52 mg, 53% yield). LCMS ESI (+) m/z 460.2 (M+H).

Step E: Preparation of 4-chloro-3-fluoro-7-(4-fluoro-2-methoxy-phenyl)thieno[3,2-c]pyridine-6-carboxylic acid: To a solution of [3-(4-fluoro-2-isopropoxy-phenyl)-6-(1-methylpyrazol-4-yl)-2-pyridyl]trifluoromethanesulfonate (31 mg, 0.068 mmol), (5-tert-butoxycarbonyl-6,7-dihydro-4H-pyrazolo[1,5-a]pyrazine-2-yl)boronic acid (36 mg, 0.14 mmol) and sodium carbonate (14 mg, 0.14 mmol) in 1,4-dioxane (1 mL) and water (0.1 mL) was added tetrakis(triphenylphosphine)palladium(0) (7.8 mg, 0.0068 mmol). The mixture was stirred at 90° C. for 2 h under Ar. The mixture was concentrated and purified by preparative TLC (DCM/MeOH 20/1) to give tert-butyl 2-[3-(4-fluoro-2-isopropoxy-phenyl)-6-(1-methylpyrazol-4-yl)-2-pyridyl]-6,7-dihydro-4H-pyrazolo[1,5-a]pyrazine-5-carboxylate (11 mg, 31% yield). LCMS ESI (+) m/z 533.2 (M+H).

Step F: Preparation of 2-[3-(4-fluoro-2-isopropoxy-phenyl)-6-(1-methylpyrazol-4-yl)-2-pyridyl]-4,5,6,7-tetrahydropyrazolo[1,5-a]pyrazine: To a solution of tert-butyl 2-[3-(4-fluoro-2-isopropoxy-phenyl)-6-(1-methylpyrazol-4-yl)-2-pyridyl]-6,7-dihydro-4H-pyrazolo[1,5-a]pyrazine-5-carboxylate (111 mg, 0.021 mmol) in DCM (1 mL) was added trifluoroacetic acid (0.33 mL). The mixture was stirred at 25° C. for 2 h. The mixture was concentrated to give crude 2-[3-(4-fluoro-2-isopropoxy-phenyl)-6-(1-methylpyrazol-4-yl)-2-pyridyl]-4,5,6,7-tetrahydropyrazolo[1,5-a]pyrazine (21 mg) which was used in the next step directly.

Step G: Preparation of 1-[2-[3-(4-fluoro-2-isopropoxy-phenyl)-6-(1-methylpyrazol-4-yl)-2-pyridyl]-6,7-dihydro-4H-pyrazolo[1,5-a]pyrazin-5-yl]prop-2-en-1-one: To a solution of 2-[3-(4-fluoro-2-isopropoxy-phenyl)-6-(1-methylpyrazol-4-yl)-2-pyridyl]-4,5,6,7-tetrahydropyrazolo[1,5-a]pyrazine (21 mg, 0.049 mmol) and triethylamine (0.017 mL, 0.097 mmol) in DCM (2 mL) was added acryloyl chloride (0.0039 mL, 0.049 mmol) under Ar. The mixture was stirred at −60° C. for 1 h. The mixture was poured into NaHCO₃ aqueous solution and the product was extracted with EtOAc. The organic layers were washed with brine, dried over anhydrous Na₂SO₄, concentrated and purified by reverse phase preparative HPLC to give 1-[2-[3-(4-fluoro-2-isopropoxy-phenyl)-6-(1-methylpyrazol-4-yl)-2-pyridyl]-6,7-dihydro-4H-pyrazolo[1,5-a]pyrazin-5-yl]prop-2-en-1-one (8.1 mg, 34% yield) as a trifluoroacetic acid salt. LCMS ESI (+) m/z 487.3 (M+H). ¹H NMR (400 MHz. CD₃OD) δ 8.52 (s, 1H), 8.27 (s, 1H), 8.16 (d, J=8.3 Hz 1H), 8.04 (d, J=8.3 Hz, 1H), 7.28 (t, J=8.0 Hz, 1H), 6.79-6.87 (m, 3H), 6.28 (d, J=16.6 Hz 1H), 5.78-5.82 (m, 2H), 4.75-4.83 (m, 2H), 4.45-4.51 (m, 1H), 4.33 (s, 2H), 4.13-4.15 (m, 2H), 4.03 (s, 3H), 1.00 (br, 6H).

### Synthetic Example 43: Synthesis of 1-[2-[2-[4-fluoro-2-(2-methoxyethoxy)phenyl]-5-(1-methylpyrazol-4-yl)-3-pyridyl]-6,7-dihydro-4H-pyrazolo[1,5-a]pyrazin-5-yl]prop-2-en-1-one (Compound 281)

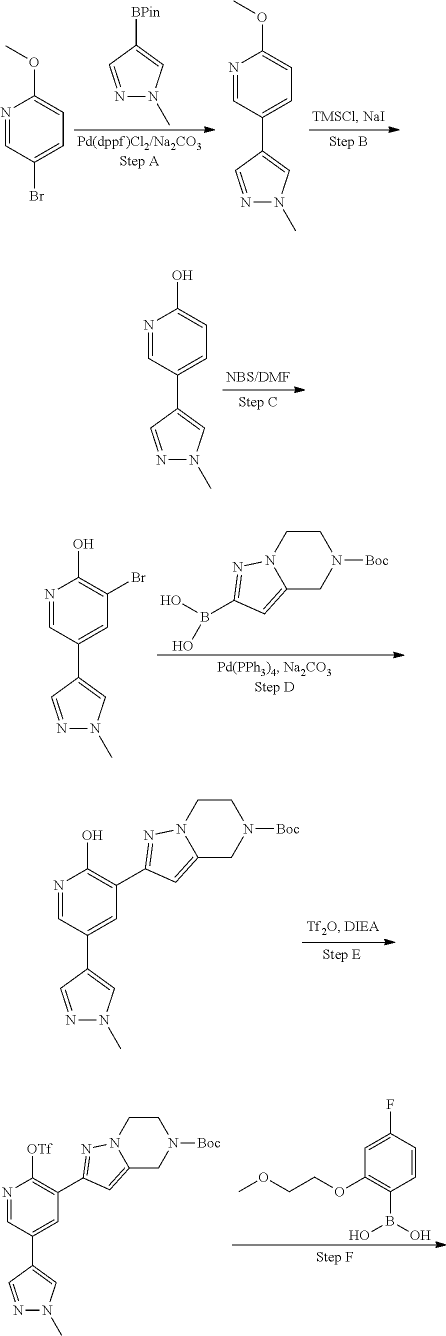

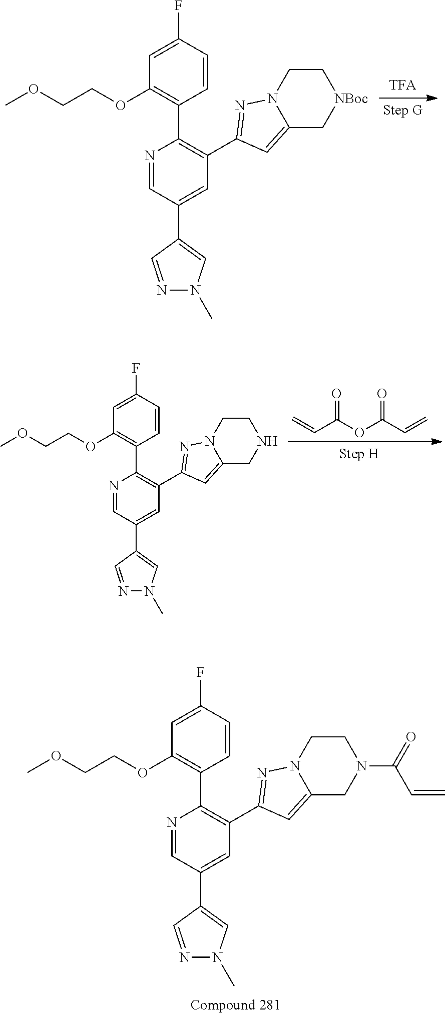

Step A: Preparation of 2-methoxy-5-(1-methylpyrazol-4-yl)pyridine: A mixture of 5-bromo-2-methoxy-pyridine (1.00 g, 5.32 mmol), 1-methyl-4-(4,4,5,5-tetramethyl-1,3,2-dioxaborolan-2-yl)pyrazole (1.22 g, 5.85 mmol), Pd(dppf)Cl₂ (389 mg, 0.532 mmol) and Na₂CO₃ (1.69 g, 16.0 mmol) in 1,4-dioxane (15 mL)/water (2 mL) was stirred at 100° C. under N₂ for 4 hours. The mixture was quenched with water and extracted with DCM. After dried and concentrated, crude product was purified by column chromatography (petroleum ether/EtOAc 1/1) to afford 2-methoxy-5-(1-methylpyrazol-4-yl)pyridine (700 mg, 70% yield). LCMS ESI (+) m/z 190.2 (M+H).

Step B: Preparation of 5-(1-methylpyrazol-4-yl) pyridin-2-ol: To a solution of 2-methoxy-5-(1-methylpyrazol-4-yl)pyridine (200 mg, 1.06 mmol) and NaI (476 mg, 3.17 mmol) in acetonitrile (8 mL) was added trimethylchlorosilane (0.41 mL, 3.2 mmol). The mixture was stirred at 70° C. for 2 h. The mixture was cooled to ambient temperature, quenched with saturated Na₂S₂O₃ solution and NaHCO₃ solution. The mixture was extracted with DCM/MeOH (9/1) twice. After dried and concentrated, the crude was purified by preparative TLC (DCM/MeOH: 19/1) to afford 5-(1-methylpyrazol-4-yl)pyridin-2-ol (45 mg, 24% yield). ¹H NMR (400M, CD₃OD) δ 7.85 (s, 1H), 7.81 (dd, J=9.4, 2.0 Hz, 1H), 7.70 (s, 1H), 7.62 (d, J=1.6 Hz, 1H), 3.90 (s, 3H).

Step C: Preparation of 3-bromo-5-(1-methylpyrazol-4-yl)pyridin-2-ol: To a solution of 5-(1-methylpyrazol-4-yl)pyridin-2-ol (20 mg, 0.11 mmol) in DMF (1.5 mL) was added N-bromosuccinimide (20 mg, 0.11 mmol) at 0° C. The mixture was stirred at 5° C. for 2 h. The mixture was quenched with saturated Na₂S₂O₃ solution and extracted with DCM/MeOH (9/1) twice. After dried and concentrated, crude was purified by preparative TLC (DCM/MeOH 15/1) to afford 3-bromo-5-(1-methylpyrazol-4-yl)pyridin-2-ol (12 mg, 41% yield). ¹H NMR (400M, CD₃OD) δ 8.22 (d, J=1.9 Hz, 1H), 7.87 (s, 1H), 7.71 (s, 1H), 7.64 (d, J=1.9 Hz, 1H), 3.90 (s, 3H).

Step D: Preparation of tert-butyl 2-(5-(1-methyl-1H-pyrazol-4-yl)-2-(((trifluoromethyl)sulfonyl)oxy)pyridin-3-yl)-6,7-dihydropyrazolo[1,5-a]pyrazine-5(4H)-carboxylate: To a solution of tert-butyl 2-[2-hydroxy-5-(1-methylpyrazol-4-yl)-3-pyridyl]-6,7-dihydro-4H-pyrazolo[1,5-a]pyrazine-5-carboxylate (30 mg, 0.076 mmol) and N,N-diisopropylethylamine (0.013 mL, 0.076 mmol) in DCM (5 mL) was added trifluoromethanesulfonic anhydride (0.026 mL, 0.15 mmol). The mixture was stirred at 25° C. for 40 min. NaHCO₃ solution was added to quench the reaction, and the mixture was stirred for additional 5 minutes. The product was extracted with DCM, dried over Na₂SO₄, concentrated and purified by silica gel column (petroleum ether:EtOAc=8:1) to give tert-butyl 2-(5-(1-methyl-1H-pyrazol-4-yl)-2-(((trifluoromethyl)sulfonyl)oxy)pyridin-3-yl)-6,7-dihydropyrazolo[1,5-a]pyrazine-5(4H)-carboxylate (30 mg, 75% yield). LCMS ESI (+) m/z 529.2 (M+H).

Step E: Preparation of tert-butyl 2-(2-(4-fluoro-2-(2-methoxyethoxy)phenyl)-5-(1-methyl-1H-pyrazol-4-yl)pyridin-3-yl)-6,7-dihydropyrazolo[1,5-a]pyrazine-5(4H)-carboxylate: A mixture of tert-butyl 2-[5-(1-methylpyrazol-4-yl)-2-(trifluoromethylsulfonyloxy)-3-pyridyl]-6,7-dihydro-4H-pyrazolo[1,5-a]pyrazine-5-carboxylate (30 mg, 0.056 mmol), [4-fluoro-2-(2-methoxyethoxy)phenyl]boronic acid (24 mg, 0.11 mmol), Pd(PPh₃)₄(10 mg) and Na₂CO₃ (12 mg, 0.11 mmol) in 1,4-dioxane (2 mL)/water (0.2 mL) was stirred at 100° C. under N₂ for 2 h. The mixture was concentrated and purified by preparative TLC (DCM/MeOH 9/1) to afford tert-butyl 2-(2-(4-fluoro-2-(2-methoxyethoxy)phenyl)-5-(1-methyl-1H-pyrazol-4-yl)pyridin-3-yl)-6,7-dihydropyrazolo[1,5-a]pyrazine-5(4H)-carboxylate (12 mg, 39% yield). LCMS ESI (+) m/z 549.4 (M+H).

Step F: Preparation of 2-[3-(4-fluoro-2-isopropoxy-phenyl)-6-(1-methylpyrazol-4-yl)-2-pyridyl]-4,5,6,7-tetrahydropyrazolo[1,5-a]pyrazine: To a solution of tert-butyl 2-[3-(4-fluoro-2-isopropoxy-phenyl)-6-(1-methylpyrazol-4-yl)-2-pyridyl]-6,7-dihydro-4H-pyrazolo[1,5-a]pyrazine-5-carboxylate (111 mg, 0.021 mmol) in DCM (1 mL) was added trifluoroacetic acid (0.33 mL). The mixture was stirred at 25° C. for 2 h. The mixture was concentrated to give crude 2-[3-(4-fluoro-2-isopropoxy-phenyl)-6-(1-methylpyrazol-4-yl)-2-pyridyl]-4,5,6,7-tetrahydropyrazolo[1,5-a]pyrazine (21 mg) which was used in the next step directly.

Step G: Preparation of 2-[2-[4-fluoro-2-(2-methoxyethoxy)phenyl]-5-(1-methylpyrazol-4-yl)-3-pyridyl]-4,5,6,7-tetrahydropyrazolo[1,5-a]pyrazine: To a solution of tert-butyl 2-[2-[4-fluoro-2-(2-methoxyethoxy)phenyl]-5-(1-methylpyrazol-4-yl)-3-pyridyl]-6,7-dihydro-4H-pyrazolo[1,5-a]pyrazine-5-carboxylate (21 mg, 0.038 mmol) in DCM (3 mL) was added trifluoroacetic acid (1.0 mL, 13.0 mmol). The mixture was stirred at 20° C. for 1 h. The mixture was concentrated to dryness to afford 2-[2-[4-fluoro-2-(2-methoxyethoxy)phenyl]-5-(1-methylpyrazol-4-yl)-3-pyridyl]-4,5,6,7-tetrahydropyrazolo[1,5-a]pyrazine (27 mg crude), which was used in next step directly. LCMS ESI (+) m/z 449.3 (M+H).

Step H: Preparation of 1-[2-[2-[4-fluoro-2-(2-methoxyethoxy)phenyl]-5-(1-methylpyrazol-4-yl)-3-pyridyl]-6,7-dihydro-4H-pyrazolo[1,5-a]pyrazin-5-yl]prop-2-en-1-one: To a solution of 2-[2-[4-fluoro-2-(2-methoxyethoxy)phenyl]-5-(1-methylpyrazol-4-yl)-3-pyridyl]-4,5,6,7-tetrahydropyrazolo[1,5-a]pyrazine (17 mg, 0.038 mmol) and DIPEA (0.033 mL, 0.19 mmol) in DCM (3 mL) was added prop-2-enoyl prop-2-enoate (9.6 mg, 0.076 mmol) at 0° C. The mixture was stirred at 0° C. for 1 h. The mixture was quenched with NaHCO₃ aqueous solution and extracted with DCM. The organic phase was dried over Na₂SO₄, filtered and concentrated. The residue was purified by reverse phase preparative HPLC to afford 1-[2-[2-[4-fluoro-2-(2-methoxyethoxy)phenyl]-5-(1-methylpyrazol-4-yl)-3-pyridyl]-6,7-dihydro-4H-pyrazolo[1,5-a]pyrazin-5-yl]prop-2-en-1-one (6.8 mg, 36% yield) as a trifluoroacetic acid salt. LCMS ESI (+) m/z 503.3 (M+H). ¹H NMR (400 MHz, CD₃OD) δ 8.96 (s, 2H), 8.35 (s, 1H), 8.13 (s, 1H), 7.41 (t, J=8.1 Hz, 1H), 7.04 (dd, J=10.9, 1.7 Hz, 1H), 6.72-6.94 (m, 2H), 6.28 (d, J=16.4 Hz, 1H), 5.67-5.84 (m, 2H), 4.70-4.85 (m, 2H), 4.25 (s, 2H), 4.12 (t, J=5.50 Hz, 2H), 4.02 (t, J=4.2 Hz, 2H), 4.00 (s, 3H), 3.46 (t, J=4.5 Hz, 2H), 3.19 (s, 3H).

### Synthetic Example 44: Synthesis of 5-[7-[4-fluoro-2-(2-methoxyethoxy)phenyl]-4-(1,2,3,4-tetrahydroisoquinolin-6-yl)thieno[3,2-c]pyridin-6-yl]-3-(1-prop-2-enoylpyrrolidin-3-yl)-1H-pyridin-2-one (Compound 70)

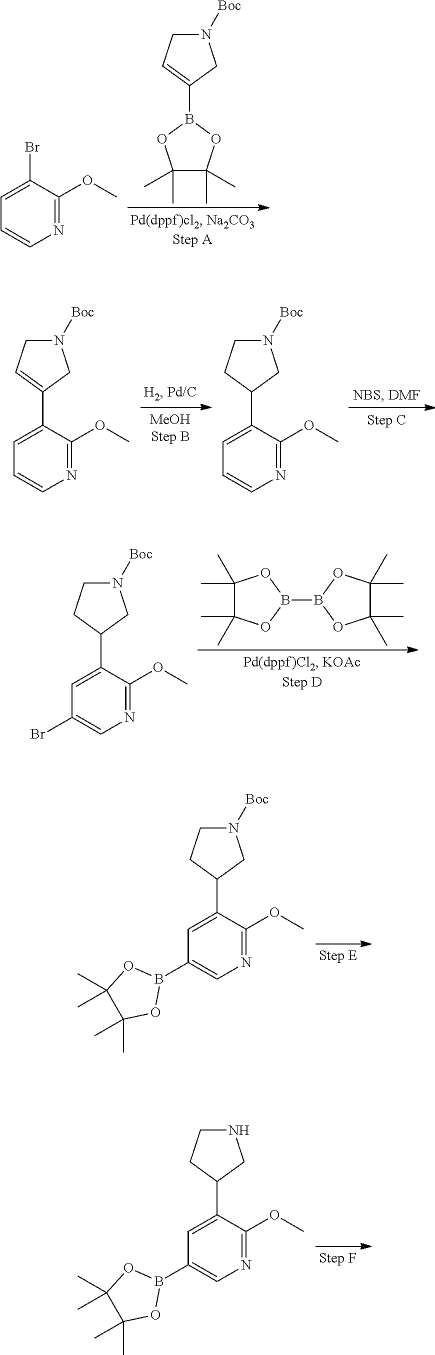

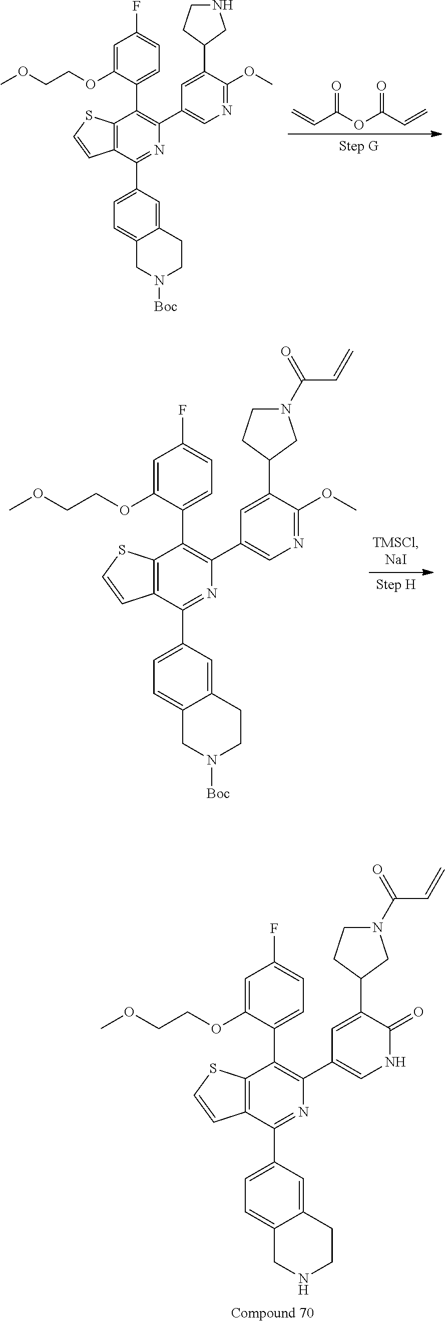

Step A: Preparation of tert-butyl 3-(2-methoxypyridin-3-yl)-2,5-dihydro-1H-pyrrole-1-carboxylate: To a solution of 3-bromo-2-methoxy-pyridine (600 mg, 3.19 mmol), tert-butyl 3-(4,4,5,5-tetramethyl-1,3,2-dioxaborolan-2-yl)-2,5-dihydropyrrole-1-carboxylate (1.04 g, 3.51 mmol) and sodium carbonate (1.02 g, 9.57 mmol) in 1,4-dioxane (6 mL) and water (2 mL) was added [1,1′-bis(diphenylphosphino)ferrocene]dichloropalladium (II) (233 mg, 0.319 mmol). The mixture was stirred at 100° C. for 2 h under Ar. The mixture was poured into water. The product was extracted with EtOAc, dried over anhydrous Na₂SO₄ and concentrated. The residue was purified by column chromatography on silica gel (EtOAc/petroleum ether: 1/3) to give tert-butyl 3-(2-methoxypyridin-3-yl)-2,5-dihydro-1H-pyrrole-1-carboxylate (600 mg, 68% yield) as a yellow oil. LCMS ESI (+) m/z 277.2 (M+H).

Step B: Preparation of tert-butyl 3-(2-methoxypyridin-3-yl)pyrrolidine-1-carboxylate: To a stirred solution of tert-butyl 3-(2-methoxy-3-pyridyl)-2,5-dihydropyrrole-1-carboxylate (600 mg, 2.17 mmol) in methanol (5 mL) was added 10% Pd/C (500 mg). The mixture was stirred at ambient temperature under H₂ (1 atm) for 1 h. The reaction mixture was filtered through Celite® and washed with ethyl acetate. The filtrate was concentrated and purified by column chromatography (EtOAc/petroleum ether: 1/3) to give tert-butyl 3-(2-methoxypyridin-3-yl)pyrrolidine-1-carboxylate (450 mg, 75% yield) as a yellow oil. LCMS ESI (+) m/z 279.2 (M+H).

Step C: Preparation of give tert-butyl 3-(5-bromo-2-methoxypyridin-3-yl)pyrrolidine-1-carboxylate: To a stirred solution of tert-butyl 3-(2-methoxy-3-pyridyl)pyrrolidine-1-carboxylate (450 mg, 1.62 mmol) in DMF (5 mL) was added N-bromosuccinimide (317 mg, 1.78 mmol). The mixture was stirred at ambient temperature for 5 hours. The mixture was poured into water and extracted twice with ethyl acetate. The combined organic phases were dried over sodium sulfate and the solvent was evaporated in vacuo. The residue was purified by silica gel column chromatography (EtOAc/petroleum ether: 1/6 to 1/4) to give tert-butyl 3-(5-bromo-2-methoxypyridin-3-yl)pyrrolidine-1-carboxylate (350 mg, 60% yield) as a yellow oil. LCMS ESI (+) m/z 357.1 (M+H).

Step D: Preparation of tert-butyl 3-(2-methoxy-5-(4,4,5,5-tetramethyl-1,3,2-dioxaborolan-2-yl)pyridin-3-yl)pyrrolidine-1-carboxylate: To a solution of tert-butyl 3-(5-bromo-2-methoxy-3-pyridyl)pyrrolidine-1-carboxylate (200 mg, 0.560 mmol) and bis(pinacolato)diboron (213 mg, 0.840 mmol), potassium acetate (110 mg, 1.12 mmol) in 1,4-dioxane (5 mL) was added [1,1′-bis(diphenylphosphino)ferrocene]dichloropalladium(II) (41 mg, 0.056 mmol). The mixture was stirred at 100° C. for 2 h under Ar. The mixture was filtered and concentrated. The residue was purified by silica gel column (EtOAc/petroleum ether: 1/6 to 1/3) to give tert-butyl 3-(2-methoxy-5-(4,4,5,5-tetramethyl-1,3,2-dioxaborolan-2-yl)pyridin-3-yl)pyrrolidine-1-carboxylate as a yellow oil (210 mg, 93% yield). LCMS ESI (+) m/z 405.3 (M+H).

Step E: Preparation of 2-methoxy-3-(pyrrolidin-3-yl)-5-(4,4,5,5-tetramethyl-1,3,2-dioxaborolan-2-yl)pyridine: To a solution of tert-butyl 3-[2-methoxy-5-(4,4,5,5-tetramethyl-1,3,2-dioxaborolan-2-yl)-3-pyridyl]pyrrolidine-1-carboxylate (200 mg, 0.495 mmol) in DCM (3 mL) was added TFA (1 mL). The mixture was stirred at 25° C. for 2 h. The mixture was concentrated to give crude 2-methoxy-3-(pyrrolidin-3-yl)-5-(4,4,5,5-tetramethyl-1,3,2-dioxaborolan-2-yl)pyridine (250 mg) as a yellow solid, which was used in the next step directly.

Step F: Preparation of tert-butyl 6-(7-(4-fluoro-2-(2-methoxyethoxy)phenyl)-6-(6-methoxy-5-(pyrrolidin-3-yl) pyridin-3-yl)thieno[3,2-c]pyridin-4-yl)-3,4-dihydroisoquinoline-2(1H)-carboxylate: To a solution of tert-butyl 6-[7-[4-fluoro-2-(2-methoxyethoxy)phenyl]-6-(trifluoromethyl-sulfonyloxy)thieno[3,2-c]pyridin-4-yl]-3,4-dihydro-1H-isoquinoline-2-carboxylate (50 mg, 0.073 mmol), 2-methoxy-3-pyrrolidin-3-yl-5-(4,4,5,5-tetramethyl-1,3,2-dioxaborolan-2-yl)pyridine (45 mg, 0.15 mmol), potassium carbonate (20 mg, 0.015 mmol) and X-PHOS (7.0 mg, 0.015 mmol) in MeCN (2 mL) and water (0.2 mL) was added palladium(II) acetate (1.7 mg, 0.0073 mmol). The mixture was stirred at 100° C. for 6 h under Ar. The mixture was poured into water. The product was extracted with EtOAc, dried over anhydrous Na₂SO₄, concentrated and purified by preparative TLC (DCM/MeOH: 10/1 containing 1% Et₃N) to give tert-butyl 6-(7-(4-fluoro-2-(2-methoxyethoxy)phenyl)-6-(6-methoxy-5-(pyrrolidin-3-yl)pyridin-3-yl)thieno[3,2-c]pyridin-4-yl)-3,4-dihydroisoquinoline-2(1H)-carboxylate (25 mg, 48% yield). LCMS ESI (+) m/z 711.3 (M+H).

Step G: Preparation of tert-butyl 6-(6-(5-(1-acryloylpyrrolidin-3-yl)-6-methoxypyridin-3-yl)-7-(4-fluoro-2-(2-methoxyethoxy)phenyl)thieno[3,2-c]pyridin-4-yl)-3,4-dihydroisoquinoline-2(1H)-carboxylate: To a solution of tert-butyl 6-(7-(4-fluoro-2-(2-methoxyethoxy)phenyl)-6-(6-methoxy-5-(pyrrolidin-3-yl)pyridin-3-yl)thieno[3,2-c]pyridin-4-yl)-3,4-dihydroisoquinoline-2(1H)-carboxylate (25 mg, 0.035 mmol) and Et₃N (10 mg, 0.10 mmol) in DCM (2 mL) was added acrylic anhydride (8.8 mg, 0.070 mmol) at −60° C. The mixture was stirred at −60° C. for 1 h. The mixture was poured into NaHCO₃ aqueous solution and extracted with EtOAc. The combined organic layers were washed with brine, dried over Na₂SO₄, filtered and concentrated to give crude tert-butyl 6-(6-(5-(1-acryloylpyrrolidin-3-yl)-6-methoxypyridin-3-yl)-7-(4-fluoro-2-(2-methoxyethoxy)phenyl)thieno[3,2-c]pyridin-4-yl)-3,4-dihydroisoquinoline-2(1H)-carboxylate (23 mg), which was used in the next step without further purification. LCMS ESI (+) m/z 765.3 (M+H).

Step H: Preparation of 5-[7-[4-fluoro-2-(2-methoxyethoxy)phenyl]-4-(1,2,3,4-tetrahydroisoquinolin-6-yl)thieno[3,2-c]pyridin-6-yl]-3-(1-prop-2-enoylpyrrolidin-3-yl)-1H-pyridin-2-one: To a suspension of tert-butyl 6-[7-[4-fluoro-2-(2-methoxyethoxy)phenyl]-6-[6-methoxy-5-(1-prop-2-enoylpyrrolidin-3-yl)-3-pyridyl]thieno[3,2-c]pyridin-4-yl]-3,4-dihydro-1H-isoquinoline-2-carboxylate (23 mg, 0.030 mmol) and sodium iodide (14 mg, 0.090 mmol) in MeCN (2 mL) was added trimethylchlorosilane (0.038 mL, 0.30 mmol). The mixture was stirred at 60° C. for 2 h. The mixture was poured into water and the product was extracted with EtOAc. The organic layers were washed with brine, dried over anhydrous Na₂SO₄, concentrated and purified by reverse phase preparative HPLC to give 5-[7-[4-fluoro-2-(2-methoxyethoxy)phenyl]-4-(1,2,3,4-tetrahydroisoquinolin-6-yl)thieno[3,2-c]pyridin-6-yl]-3-(1-prop-2-enoylpyrrolidin-3-yl)-1H-pyridin-2-one (3.6 mg, 18% yield) as a trifluoroacetic acid salt. LCMS ESI (+) m/z 651.3 (M+H). ¹H NMR (400 MHz, CD₃OD) δ 7.81 (d, J=9.7 Hz, 2H), 7.74 (d, J=5.6 Hz, 1H), 7.62 (dd, J=5.6, 1.6 Hz, 1H), 7.50-7.58 (m, 2H), 7.45 (d, J=7.7 Hz, 1H), 7.32-7.39 (m, 1H), 6.97-7.02 (m, 1H), 6.84-6.90 (m, 1H), 6.52-6.63 (m, 1H), 6.23-6.30 (m, 1H), 5.71-5.76 (m, 1H), 4.49 (s, 2H), 3.83-4.05 (m, 3H), 3.64-3.67 (m, 1H), 3.59 (t, J=6.3 Hz, 3H), 3.44-3.53 (m, 4H), 3.28 (t, J=6.3 Hz, 2H), 3.10 (t, J=2.8 Hz, 3H), 2.12-2.28 (m, 1H), 1.81-1.88 (m, 1H).

### Synthetic Example 45: Synthesis of 5-[7-(2,4-difluoro-6-isopropoxy-phenyl)-6-(5-prop-2-enoyl-6,7-dihydro-4H-thiazolo[5,4-c]pyridin-2-yl)thieno[3,2-c]pyridin-4-yl]isoindolin-1-one (Compound 311)

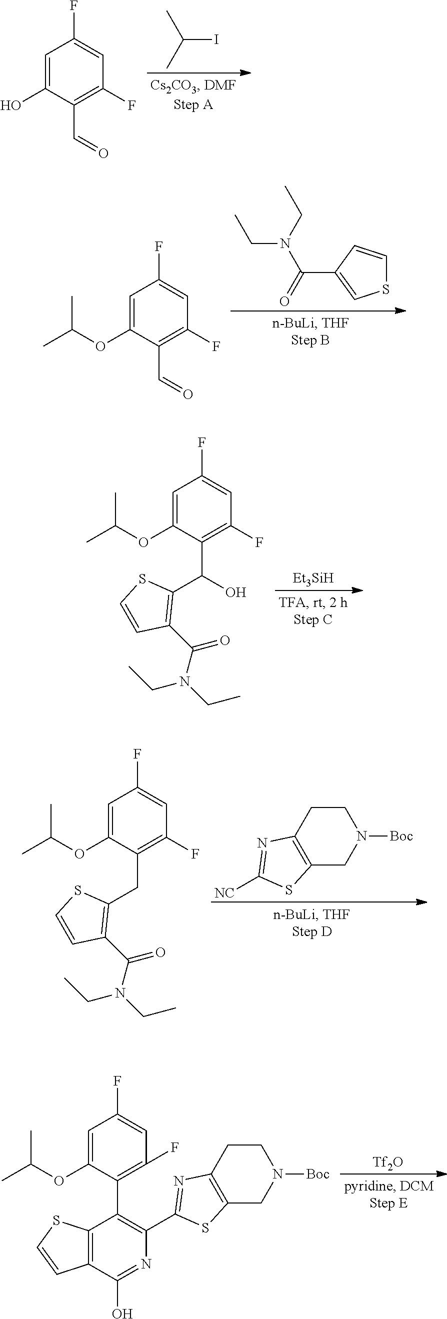

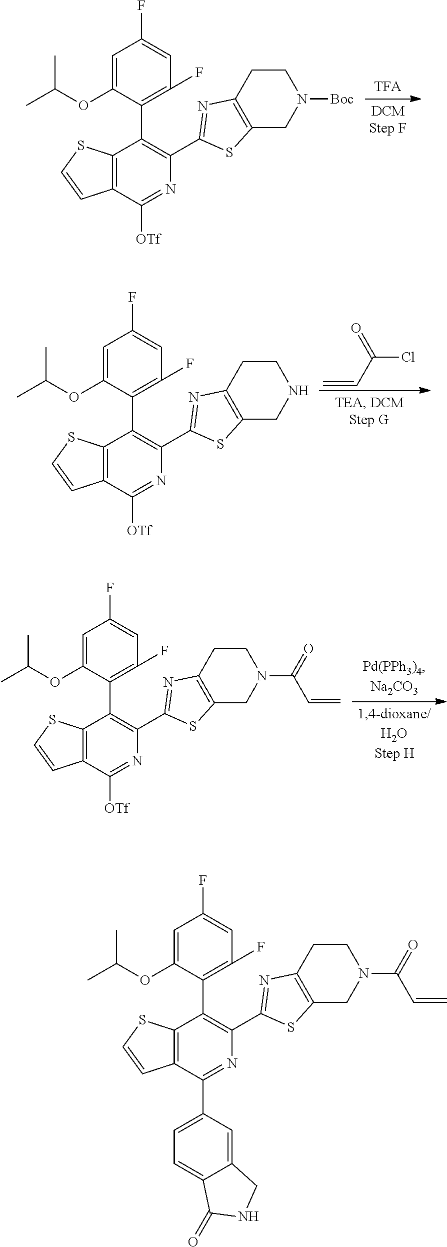

Step A: Preparation of 2,4-difluoro-6-isopropoxy-benzaldehyde: To a solution of 2,4-difluoro-6-hydroxy-benzaldehyde (7.00 g, 44.3 mmol) and cesium carbonate (28.9 g, 88.6 mmol) in DMF (120 mL) was added 2-iodopropane (6.60 mL, 66.4 mmol) under argon. The resulting mixture was stirred at 50° C. for 3 hours. The reaction was concentrated to dryness and the residue was taken up in EtOAc. The organics were washed with water and brine, dried and concentrated. The crude was then purified by column chromatography on silica gel (20% EtOAc in hexanes) to give 2,4-difluoro-6-isopropoxy-benzaldehyde (8.20 g, 93% yield) as yellow solid. LCMS ESI (+) m/z 201.1 (M+H).

Step B: Preparation of 2-[(2,4-difluoro-6-isopropoxy-phenyl)-hydroxy-methyl]-N,N-diethyl-thiophene-3-carboxamide: To a solution of N,N-diethylthiophene-3-carboxamide (3.32 g, 18.1 mmol) in THF (60 mL) was added n-BuLi (8.0 mL, 2.5 M in hexanes, 19.9 mmol) at −60° C. The mixture was stirred at −60° C. for 30 min. 2,4-Difluoro-6-isopropoxy-benzaldehyde (3.99 g, 19.9 mmol) in THF (20 mL) was added and the resulting mixture was stirred at 25° C. for additional 1 h. The reaction was poured into water and the mixture was extracted with EtOAc. The combined organics were washed with saturated brine solution. The organics were then separated and dried (MgSO₄) before concentration to dryness. The crude was then purified by column chromatography on silica gel (20% EtOAc in hexanes) to give 2-[(2,4-difluoro-6-isopropoxy-phenyl)-hydroxy-methyl]-N,N-diethyl-thiophene-3-carboxamide (6.10 g, 88% yield). LCMS ESI (−) m/z 361.1 (M-17).

Step C: Preparation of 2-[(2,4-difluoro-6-isopropoxy-phenyl)methyl]-N,N-diethyl-thiophene-3-carboxamide: To a solution of 2-[(2,4-difluoro-6-isopropoxy-phenyl)-hydroxy-methyl]-N,N-diethyl-thiophene-3-carboxamide (760 mg, 1.98 mmol) in TFA (11 mL, 148 mmol) was added triethylsilane (3.20 mL, 19.8 mmol). The mixture was stirred at 25° C. for 2 h. The reaction was concentrated to dryness and diluted with EtOAc (60 mL). The solution was washed with saturated aqueous NaHCO₃ solution and saturated brine solution. The organics were then separated and dried (MgSO₄) before concentration to dryness. The crude was then purified by column chromatography eluting with 10% EtOAc in hexanes to give 2-[(2,4-difluoro-6-isopropoxy-phenyl)methyl]-N,N-diethyl-thiophene-3-carboxamide (660 mg, 91% yield). ¹H NMR (400 MHz, CDCl₃) 7.04 (d, J=5.2 Hz, 1H), 6.85 (d, J=5.2 Hz, 1H), 6.34-6.43 (m, 2H), 4.50-4.58 (m, 1H), 4.11 (s, 2H), 3.51-3.62 (m, 2H), 3.15-3.28 (m, 2H), 1.35 (d, J=6.0 Hz, 6H) 1.20-1.32 (m, 3H), 1.01-1.13 (m, 3H). LCMS ESI (+) m/z 368.1 (M+H).

Step D: Preparation of tert-butyl 2-[7-(2,4-difluoro-6-isopropoxy-phenyl)-4-hydroxy-thieno[3,2-c]pyridin-6-yl]-6,7-dihydro-4H-thiazolo[5,4-c]pyridine-5-carboxylate: To a solution of 2-[(2,4-difluoro-6-isopropoxy-phenyl)methyl]-N,N-diethyl-thiophene-3-carboxamide (200 mg, 0.544 mmol) in THE (5 mL) was added n-BuLi (0.26 mL, 2.5 M in hexanes, 0.65 mmol) dropwise under Ar at −60° C. The mixture was stirred at −60° C. for 0.5 h under Ar. A solution of tert-butyl 2-cyano-6,7-dihydro-4H-thiazolo[5,4-c]pyridine-5-carboxylate (130 mg, 0.490 mmol) in THE (1 mL) was added at −60° C. The temperature was allowed to warm to 0° C., and the resulting mixture was stirred at 0° C. for additional 1 h. The mixture was poured into water and 1 N aqueous HCl was added to adjust the pH to 5. The mixture was extracted with EtOAc, dried over anhydrous Na₂SO₄, concentrated in vacuo and purified by preparative TLC (DCM/MeOH=20/1) to give tert-butyl 2-[7-(2,4-difluoro-6-isopropoxy-phenyl)-4-hydroxy-thieno[3,2-c]pyridin-6-yl]-6,7-dihydro-4H-thiazolo[5,4-c]pyridine-5-carboxylate (113 mg, 37% yield). LCMS ESI (+) m/z 560.3 (M+H).

Step E: Preparation of tert-butyl 2-[7-(2,4-difluoro-6-isopropoxy-phenyl)-4-(trifluoromethylsulfonyloxy)thieno[3,2-c]pyridin-6-yl]-6,7-dihydro-4H-thiazolo[5,4-c]pyridine-5-carboxylate: To a solution of tert-butyl 2-[7-(2,4-difluoro-6-isopropoxy-phenyl)-4-hydroxy-thieno[3,2-c]pyridin-6-yl]-6,7-dihydro-4H-thiazolo[5,4-c]pyridine-5-carboxylate (120 mg, 0.214 mmol) and pyridine (0.087 mL, 1.1 mmol) in DCM (5 mL) was added trifluoromethanesulfonic anhydride (0.072 mL, 0.43 mmol). The mixture was stirred at 25° C. for 0.5 h. The mixture was poured into water and the product was extracted with EtOAc. The organics were washed with brine, dried over anhydrous Na₂SO₄, concentrated and purified by preparative TLC (EtOAc/petroleum ether: 1/5) to give tert-butyl 2-[7-(2,4-difluoro-6-isopropoxy-phenyl)-4-(trifluoromethylsulfonyloxy)thieno[3,2-c]pyridin-6-yl]-6,7-dihydro-4H-thiazolo[5,4-c]pyridine-5-carboxylate (115 mg, 78% yield). LCMS ESI (+) m/z 692.1 (M+H).

Step F: Preparation of [7-(2,4-difluoro-6-isopropoxy-phenyl)-6-(4,5,6,7-tetrahydrothiazolo[5,4-c]pyridin-2-yl)thieno[3,2-c]pyridin-4-yl]trifluoromethanesulfonate: To a solution of tert-butyl 2-[7-(2,4-difluoro-6-isopropoxy-phenyl)-4-(trifluoromethylsulfonyloxy)thieno[3,2-c]pyridin-6-yl]-6,7-dihydro-4H-thiazolo[5,4-c]pyridine-5-carboxylate (55 mg, 0.080 mmol) in dichloromethane (3 mL) was added trifluoroacetic acid (1 mL). The mixture was stirred at 25° C. for 1 h. The solvent was concentrated to dryness under reduced pressure to afford [7-(2,4-difluoro-6-isopropoxy-phenyl)-6-(4,5,6,7-tetrahydrothiazolo[5,4-c]pyridin-2-yl)thieno[3,2-c]pyridin-4-yl]trifluoromethanesulfonate trifluoroacetic acid salt (62 mg, 100% yield) which was used in the next step directly. LCMS ESI (+) m/z 591.8 (M+H).

Step G: Preparation of [7-(2,4-difluoro-6-isopropoxy-phenyl)-6-(5-prop-2-enoyl-6,7-dihydro-4H-thiazolo[5,4-c]pyridin-2-yl)benzothiophen-4-yl]trifluoromethanesulfonate: To a solution of [7-(2,4-difluoro-6-isopropoxy-phenyl)-6-(4,5,6,7-tetrahydrothiazolo[5,4-c]pyridin-2-yl)thieno[3,2-c]pyridin-4-yl]trifluoromethanesulfonate trifluoroacetic acid salt (62 mg, 0.088 mmol) in DCM (2 mL) was added triethylamine (0.099 mL, 0.71 mmol), followed by acryloyl chloride (0.014 mL, 0.18 mmol) at −60° C. The mixture was stirred at −60° C. for 15 min. The mixture was poured into NaHCO₃ aqueous solution and extracted with EtOAc. The combined organic layers were washed with brine, dried over anhydrous Na₂SO₄, filtered and concentrated to dryness to give crude [7-(2,4-difluoro-6-isopropoxy-phenyl)-6-(5-prop-2-enoyl-6,7-dihydro-4H-thiazolo[5,4-c]pyridin-2-yl)benzothiophen-4-yl]trifluoromethanesulfonate (48 mg, 85% yield) which was used directly in the next step. LCMS ESI (+) m/z 646.0 (M+H).

Step H: Preparation of [7-(2,4-difluoro-6-isopropoxy-phenyl)-6-(5-prop-2-enoyl-6,7-dihydro-4H-thiazolo[5,4-c]pyridin-2-yl)benzothiophen-4-yl]trifluorometha5-[7-(2,4-difluoro-6-isopropoxy-phenyl)-6-(5-prop-2-enoyl-6,7-dihydro-4H-thiazolo[5,4-c]pyridin-2-yl)thieno[3,2-c]pyridin-4-yl]isoindolin-1-one: To a solution of [7-(2,4-difluoro-6-isopropoxy-phenyl)-6-(5-prop-2-enoyl-6,7-dihydro-4H-thiazolo[5,4-c]pyridin-2-yl)thieno[3,2-c]pyridin-4-yl]trifluoromethanesulfonate (16 mg, 0.025 mmol), sodium carbonate (10 mg, 0.098 mmol), and 5-(4,4,5,5-tetramethyl-1,3,2-dioxaborolan-2-yl)isoindolin-1-one (8.6 mg, 0.033 mmol) in 1,4-dioxane (2 mL) and water (0.2 mL) was added tetrakis(triphenylphosphine) palladium(0) (3.0 mg, 0.0026 mmol). The mixture was stirred at 100° C. for 1 h under Ar. After cooling, the reaction mixture was filtered and concentrated. The crude was purified by preparative TLC (DCM/MeOH: 20/1) followed by preparative HPLC to give 5-[7-(2,4-difluoro-6-isopropoxy-phenyl)-6-(5-prop-2-enoyl-6,7-dihydro-4H-thiazolo[5,4-c]pyridin-2-yl)thieno[3,2-c]pyridin-4-yl]isoindolin-1-one (6.8 mg, 43% yield). ¹H NMR (400 MHz, CD₃OD) 8.20 (s, 1H), 8.14 (d, J=8.0 Hz, 1H), 8.01 (d, J=8.0 Hz, 1H), 7.83 (d, J=5.4 Hz, 1H), 7.76 (d, J=5.4 Hz, 1H), 6.74-6.90 (m, 2H), 6.66 (d, J=8.8 Hz, 1H), 6.24 (d, J=16.8 Hz, 1H) 5.79 (d, J=10.2 Hz, 1H), 4.63 (s, 2H), 4.44-4.53 (m, 1H), 3.85-4.00 (m, 2H), 2.67-2.71 (m, 2H), 1.07 (d, J=5.6 Hz, 3H), 0.89 (d, J=5.6 Hz, 3H). LCMS ESI (+) m/z 629.2 (M+H).

### Synthetic Example 46: Synthesis of 1-[2-[7-(4-fluoro-2-isopropoxy-phenyl)-4-(4-methylpyrazol-1-yl)thieno[3,2-c]pyridin-6-yl]-6,7-dihydro-4H-pyrazolo[1,5-a]pyrazin-5-yl]prop-2-en-1-one (Compound 317)

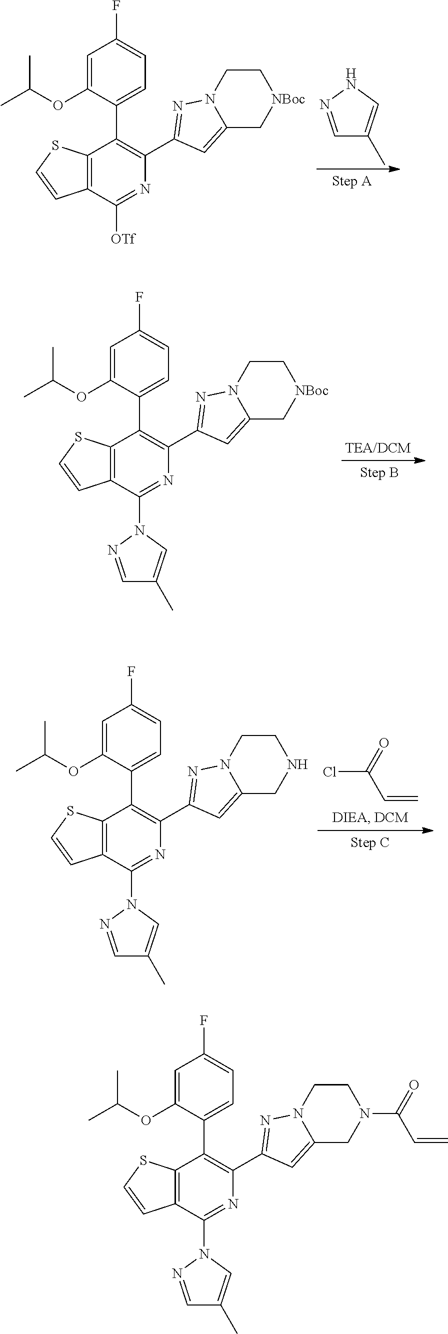

Step A: Preparation of tert-butyl 2-[7-(4-fluoro-2-isopropoxy-phenyl)-4-(4-methylpyrazol-1-yl)thieno[3,2-c]pyridin-6-yl]-6,7-dihydro-4H-pyrazolo[1,5-a]pyrazine-5-carboxylate: A suspension of tert-butyl 2-[7-(4-fluoro-2-isopropoxy-phenyl)-4-(trifluoromethylsulfonyloxy)thieno[3,2-c]pyridin-6-yl]-6,7-dihydro-4H-pyrazolo[1,5-a]pyrazine-5-carboxylate (32 mg, 0.049 mmol), 4-methyl-1H-pyrazole (8.7 mg, 0.11 mmol), potassium phosphate (21 mg, 0.099 mmol), tris(dibenzylideneacetone)dipalladium(0) (4.2 mg, 0.0050 mmol) and t-butylBrettPhos (5.0 mg, 0.010 mmol) in 1,4-dioxane (1 mL) was stirred at 70° C. for 6 hours. The mixture was concentrated and purified by preparative TLC (EtOAc/petroleum ether: 1/2) to give tert-butyl 2-[7-(4-fluoro-2-isopropoxy-phenyl)-4-(4-methylpyrazol-1-yl)thieno[3,2-c]pyridin-6-yl]-6,7-dihydro-4H-pyrazolo[1,5-a]pyrazine-5-carboxylate (20 mg, 70% yield).

Step B: Preparation of 7-(4-fluoro-2-isopropoxyphenyl)-4-(4-methyl-1H-pyrazol-1-yl)-6-(4,5,6,7-tetrahydropyrazolo[1,5-a]pyrazin-2-yl)thieno[3,2-c]pyridine: To a solution of tert-butyl 2-[7-(4-fluoro-2-isopropoxy-phenyl)-4-(4-methylpyrazol-1-yl)thieno[3,2-c]pyridin-6-yl]-6,7-dihydro-4H-pyrazolo[1,5-a]pyrazine-5-carboxylate (20 mg, 0.034 mmol) in DCM (3 mL) was added trifluoroacetic acid (1.0 mL, 13 mmol) under Ar. The reaction was stirred at 24° C. for 30 minutes. The reaction was concentrated to dryness to give crude 7-(4-fluoro-2-isopropoxyphenyl)-4-(4-methyl-1H-pyrazol-1-yl)-6-(4,5,6,7-tetrahydropyrazolo[1,5-a]pyrazin-2-yl)thieno[3,2-c]pyridine trifluoroacetic acid salt (35 mg), which was used directly in the next step. LCMS ESI (+) m/z 489.3 (M+H).

Step C: Preparation of 1-[2-[7-(4-fluoro-2-isopropoxy-phenyl)-4-(4-methylpyrazol-1-yl)thieno [3,2-c]pyridin-6-yl]-6,7-dihydro-4H-pyrazolo[1,5-a]pyrazin-5-yl]prop-2-en-1-one: To a mixture of 7-(4-fluoro-2-isopropoxy-phenyl)-4-(4-methylpyrazol-1-yl)-6-(4,5,6,7-tetrahydropyrazolo[1,5-a]pyrazin-2-yl)thieno[3,2-c]pyridine (35 mg, 0.057 mmol) and N,N-diisopropylethylamine (0.020 mL, 0.12 mmol) in DCM (1 mL) cooled to −60° C. was added acryloyl chloride (0.0090 mL, 0.12 mmol) under Ar. The mixture was stirred at this temperature for 1 hour. The reaction was diluted with DCM and poured into saturated NaHCO₃ aqueous solution and stirred for 10 minutes. The organics were then separated and dried (Na₂SO₄) before concentration to dryness. The crude was then purified by preparative HPLC to give 1-[2-[7-(4-fluoro-2-isopropoxy-phenyl)-4-(4-methylpyrazol-1-yl)thieno[3,2-c]pyridin-6-yl]-6,7-dihydro-4H-pyrazolo[1,5-a]pyrazin-5-yl]prop-2-en-1-one (5.8 mg, 19% yield) as a trifluoroacetic acid salt. ¹H NMR (400 MHz, CD₃OD) δ 8.76 (s, 1H), 8.46 (d, J=5.5 Hz, 1H), 7.65-7.72 (m, 2H), 7.32 (t, J=7.6 Hz, 1H), 6.77-6.92 (m, 3H), 6.23-6.32 (m, 1H), 5.78-5.86 (m, 2H), 4.71-4.78 (m, 1H), 4.41-4.50 (m, 1H), 4.17-4.26 (m, 2H), 4.08-4.15 (m, 2H), 2.23 (s, 3H), 1.05 (d, J=5.9 Hz, 3H), 0.88 (d, J=5.8 Hz, 3H). LCMS ESI (+) m/z 543.2 (M+H).

### Synthetic Example 47: Synthesis of 1-[2-[7-(4-fluoro-2-isopropoxy-phenyl)-4-(4-methylpyrazol-1-yl)thieno[3,2-c]pyridin-6-yl]-6,7-dihydro-4H-pyrazolo[1,5-a]pyrazin-5-yl]prop-2-en-1-one (Compound 322)

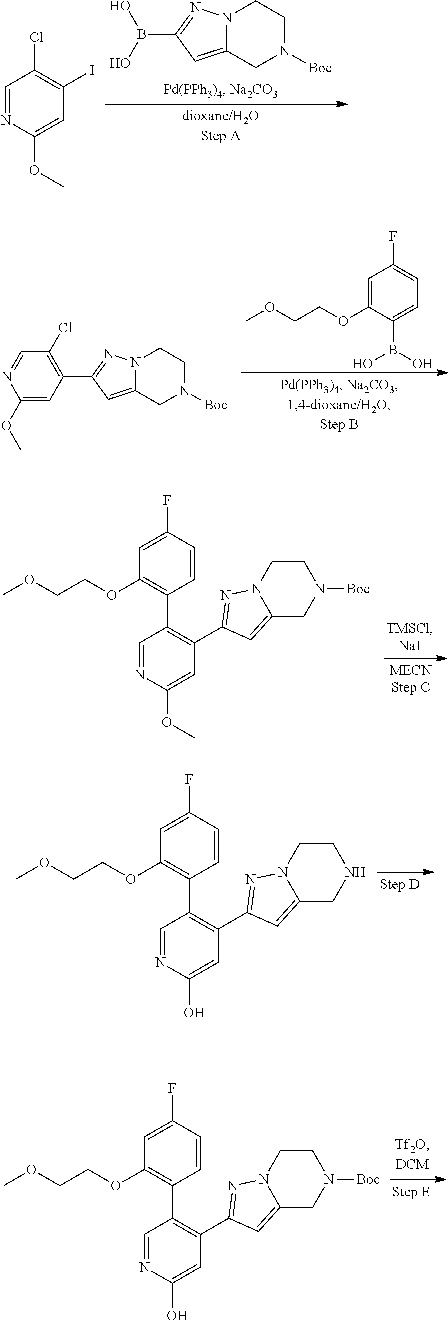

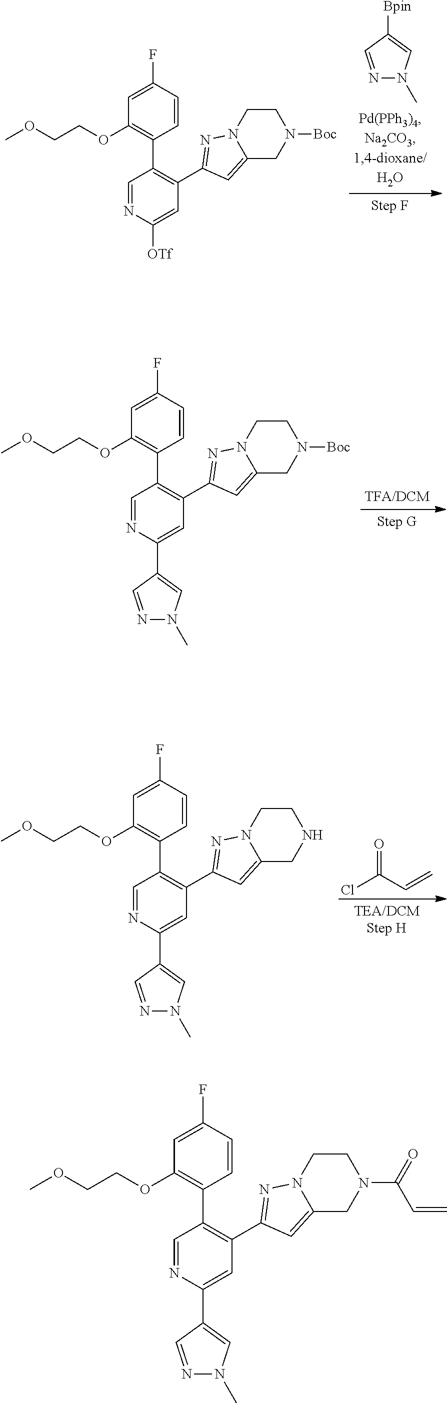

Step A: Preparation of tert-butyl 2-(5-chloro-2-methoxy-4-pyridyl)-6,7-dihydro-4H-pyrazolo[1,5-a]pyrazine-5-carboxylate: To a solution of (5-tert-butoxycarbonyl-6,7-dihydro-4H-pyrazolo[1,5-a]pyrazin-2-yl)boronic acid (357 mg, 1.34 mmol), 5-chloro-4-iodo-2-methoxy-pyridine (300 mg, 1.11 mmol) and Na₂CO₃ (295 mg, 2.78 mmol) in 1,4-dioxane (5 mL) and water (0.5 mL) was added Pd(PPh₃)₄(129 mg, 0.111 mmol). The mixture was stirred at 70° C. for 6 h under Ar. The mixture was poured into water and the product was extracted with EtOAc, dried over anhydrous Na₂SO₄, concentrated and purified by preparative TLC (petroleum ether/EtOAc=3/1) to give tert-butyl 2-(5-chloro-2-methoxy-4-pyridyl)-6,7-dihydro-4H-pyrazolo[1,5-a]pyrazine-5-carboxylate (170 mg, 42% yield).

Step B: Preparation of tert-butyl 2-[5-[4-fluoro-2-(2-methoxyethoxy)phenyl]-2-methoxy-4-pyridyl]-6,7-dihydro-4H-pyrazolo[1,5-a]pyrazine-5-carboxylate: To a solution of tert-butyl 2-(5-chloro-2-methoxy-4-pyridyl)-6,7-dihydro-4H-pyrazolo[1,5-a]pyrazine-5-carboxylate (120 mg, 0.329 mmol), [4-fluoro-2-(2-methoxyethoxy)phenyl]boronic acid (70 mg, 0.33 mmol), and K₃PO₄ (209 mg, 0.987 mmol) in 1,4-dioxane (2 mL) and water (0.2 mL) was added XPhos-Pd-G2 (26 mg, 0.033 mmol). The mixture was stirred at 105° C. for 16 h under Ar. After cooling, the mixture was concentrated and purified by preparative TLC (EtOAc/petroleum ether: 1/3) to give tert-butyl 2-[5-[4-fluoro-2-(2-methoxyethoxy)phenyl]-2-methoxy-4-pyridyl]-6,7-dihydro-4H-pyrazolo[1,5-a]pyrazine-5-carboxylate (41 mg, 25% yield). LCMS ESI (+) m/z 499.4 (M+H).

Step C: Preparation of 5-(4-fluoro-2-(2-methoxyethoxy)phenyl)-4-(4,5,6,7-tetrahydropyrazolo[1,5-a]pyrazin-2-yl)pyridin-2-ol: To a solution of tert-butyl 2-[5-[4-fluoro-2-(2-methoxyethoxy)phenyl]-2-methoxy-4-pyridyl]-6,7-dihydro-4H-pyrazolo[1,5-a]pyrazine-5-carboxylate (55 mg, 0.11 mmol) and sodium iodide (165 mg, 1.10 mmol) in MeCN (2 mL) was added trimethylchlorosilane (0.14 mL, 1.1 mmol). The mixture was stirred at 25° C. for 28 h. The mixture was poured into aqueous Na₂SO₃ solution and the product was extracted with EtOAc. The organics were washed with brine, dried over anhydrous Na₂SO₄. The mixture was concentrated in vacuo to give crude 5-(4-fluoro-2-(2-methoxyethoxy)phenyl)-4-(4,5,6,7-tetrahydropyrazolo[1,5-a]pyrazin-2-yl)pyridin-2-ol which was used in the next step directly.

Step D: Preparation of tert-butyl 2-[5-[4-fluoro-2-(2-methoxyethoxy)phenyl]-2-hydroxy-4-pyridyl]-6,7-dihydro-4H-pyrazolo[1,5-a]pyrazine-5-carboxylate: To a mixture of crude 5-(4-fluoro-2-(2-methoxyethoxy)phenyl)-4-(4,5,6,7-tetrahydropyrazolo[1,5-a]pyrazin-2-yl)pyridin-2-ol in THF (3 mL), methanol (6 mL) and water (10 mL) was added Boc₂O (102 mg, 0.468 mmol) and NaOH (75 mg, 1.9 mmol). The mixture was stirred at 25° C. for 2 h. The pH of the mixture was adjusted to 5 with 1 N HCl. The product was extracted with EtOAc. The combined organics were washed with brine, dried over anhydrous Na₂SO₄, concentrated and purified by preparative TLC (EtOAc/petroleum ether: 1/1) to give tert-butyl 2-[5-[4-fluoro-2-(2-methoxyethoxy)phenyl]-2-hydroxy-4-pyridyl]-6,7-dihydro-4H-pyrazolo[1,5-a]pyrazine-5-carboxylate (5.3 mg, 10% yield).

Step E: Preparation of tert-butyl 2-[3-[4-fluoro-2-(2-methoxyethoxy)phenyl]-6-(trifluoromethylsulfonyloxy)-2-pyridyl]-6,7-dihydro-4H-pyrazolo[1,5-a]pyrazine-5-carboxylate: To a solution of tert-butyl 2-[3-[4-fluoro-2-(2-methoxyethoxy)phenyl]-6-hydroxy-2-pyridyl]-6,7-dihydro-4H-pyrazolo[1,5-a]pyrazine-5-carboxylate (5.3 mg, 0.011 mmol) and pyridine (0.025 mL, 0.30 mmol) in DCM (2 mL) was added trifluoromethanesulfonic anhydride (0.0054 mL, 0.032 mmol). The mixture was stirred at 15° C. for 1 h. The mixture was concentrated and diluted with EtOAc, washed with brine, dried over anhydrous Na₂SO₄ and concentrated in vacuo to give tert-butyl 2-[3-[4-fluoro-2-(2-methoxyethoxy)phenyl]-6-(trifluoromethylsulfonyloxy)-2-pyridyl]-6,7-dihydro-4H-pyrazolo[1,5-a]pyrazine-5-carboxylate (7.0 mg, 83% yield).

Step F: Preparation of tert-butyl 2-[3-[4-fluoro-2-(2-methoxyethoxy)phenyl]-6-(1-methylpyrazol-4-yl)-2-pyridyl]-6,7-dihydro-4H-pyrazolo[1,5-a]pyrazine-5-carboxylate: To a solution of tert-butyl 2-[3-[4-fluoro-2-(2-methoxyethoxy)phenyl]-6-(trifluoromethylsulfonyloxy)-2-pyridyl]-6,7-dihydro-4H-pyrazolo[1,5-a]pyrazine-5-carboxylate (7.0 mg, 0.0091 mmol), sodium carbonate (4.0 mg, 0.038 mmol), and 1-methyl-4-(4,4,5,5-tetramethyl-1,3,2-dioxaborolan-2-yl)pyrazole (4.0 mg, 0.019 mmol) in 1,4-dioxane (2 mL) and water (0.2 mL) was added tetrakis(triphenylphosphine)palladium(0) (1.5 mg, 0.0013 mmol). The mixture was stirred at 100° C. for 1 h under Ar. The mixture was concentrated and purified by Preparative TLC (DCM/MeOH:30/1) to give tert-butyl 2-[3-[4-fluoro-2-(2-methoxyethoxy)phenyl]-6-(1-methylpyrazol-4-yl)-2-pyridyl]-6,7-dihydro-4H-pyrazolo[1,5-a]pyrazine-5-carboxylate (6.1 mg, 86% yield).

Step G: Preparation of 2-[3-[4-fluoro-2-(2-methoxyethoxy)phenyl]-6-(1-methylpyrazol-4-yl)-2-pyridyl]-4,5,6,7-tetrahydropyrazolo[1,5-a]pyrazine: To a solution of tert-butyl 2-[3-[4-fluoro-2-(2-methoxyethoxy)phenyl]-6-(1-methylpyrazol-4-yl)-2-pyridyl]-6,7-dihydro-4H-pyrazolo[1,5-a]pyrazine-5-carboxylate (6.1 mg, 0.0078 mmol) in DCM (1 mL) was added trifluoroacetic acid (0.30 mL, 3.9 mmol). The mixture was stirred at 15° C. for 2 h. The mixture was concentrated to dryness in vacuo to give 2-[3-[4-fluoro-2-(2-methoxyethoxy)phenyl]-6-(1-methylpyrazol-4-yl)-2-pyridyl]-4,5,6,7-tetrahydropyrazolo[1,5-a]pyrazine (8.0 mg) as a trifluoroacetic acid salt, which was used in the next step directly. LCMS ESI (+) m/z 449.2 (M+H).

Step H: Preparation of 1-[2-[3-[4-fluoro-2-(2-methoxyethoxy)phenyl]-6-(1-methylpyrazol-4-yl)-2-pyridyl]-6,7-dihydro-4H-pyrazolo[1,5-a]pyrazin-5-yl]prop-2-en-1-one: To a solution of 2-[3-[4-fluoro-2-(2-methoxyethoxy)phenyl]-6-(1-methylpyrazol-4-yl)-2-pyridyl]-4,5,6,7-tetrahydropyrazolo[1,5-a]pyrazine trifluoroacetic acid salt (8.0 mg, 0.012 mmol) in DCM (12 mL) was added triethylamine (0.022 mL, 0.16 mmol), followed by acryloyl chloride (2.1 mg, 0.023 mmol) at −60° C. The mixture was stirred at −60° C. for 15 min. The mixture was poured into NaHCO₃ aqueous solution. The product was extracted with EtOAc, washed with brine solution, dried over anhydrous Na₂SO₄, concentrated and purified by Preparative HPLC to give 1-[2-[3-[4-fluoro-2-(2-methoxyethoxy)phenyl]-6-(1-methylpyrazol-4-yl)-2-pyridyl]-6,7-dihydro-4H-pyrazolo[1,5-a]pyrazin-5-yl]prop-2-en-1-one (2.7 mg, 41% yield) as a bis trifluoroacetic acid salt. ¹H NMR (400 MHz, CD₃OD) δ 8.51 (s, 1H), 8.47 (s, 1H), 8.42 (s, 1H), 8.22 (s, 1H), 7.31 (t, J=7.8 Hz, 1H), 6.71-6.97 (m, 3H), 6.28 (d, J=16.8 Hz, 1H), 5.77-5.86 (m, 1H), 5.65-5.75 (m, 1H), 4.69-4.85 (m, 2H), 4.23-4.35 (m, 2H), 4.10-4.18 (m, 2H), 4.03 (s, 3H), 3.92-4.00 (m, 2H), 3.41 (t, J=4.4 Hz, 2H), 3.17 (s, 3H). LCMS ESI (+) m/z 503.2 (M+H).

### Synthetic Example 48: Synthesis of 5-[5-(4-fluoro-2-isopropoxy-phenyl)-4-methyl-6-(5-prop-2-enoyl-6,7-dihydro-4H-pyrazolo[1,5-a]pyrazin-2-yl)pyrimidin-2-yl]-1-methyl-pyridin-2-one (Compound 331)

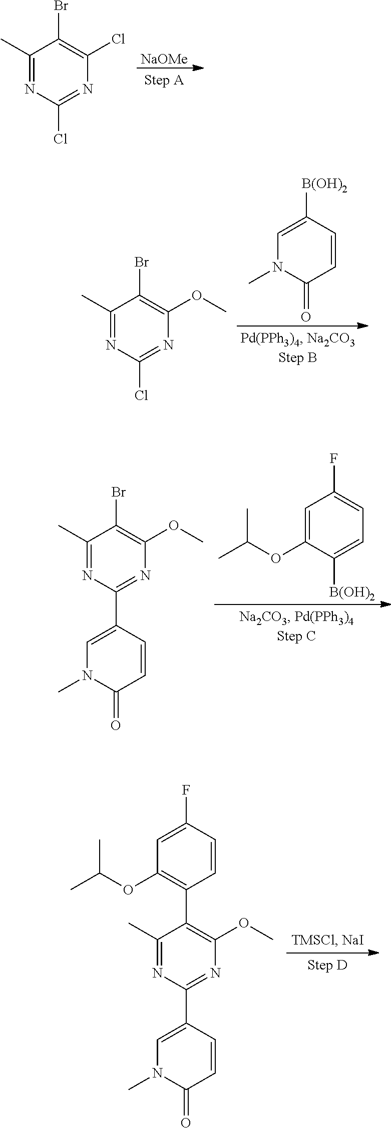

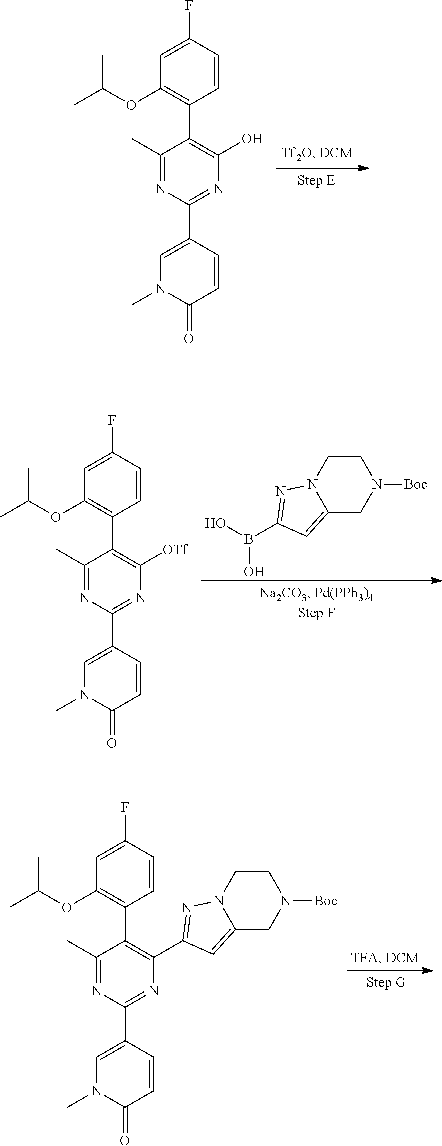 [SMILES: Cc1nc(-c2ccc(=O)n(C)c2)nc(O)c1-c1ccc(F)cc1OC(C)C ; Cc1nc(-c2ccc(=O)n(C)c2)nc(OS(=O)(=O)C(F)(F)F)c1-c1ccc(F)cc1OC(C)C ; CC(C)(C)OC(=O)N1CCn2nc(B(O)O)cc2C1 ; Cc1nc(-c2ccc(=O)n(C)c2)nc(-c2cc3n(n2)CCN(C(=O)OC(C)(C)C)C3)c1-c1ccc(F)cc1OC(C)C]

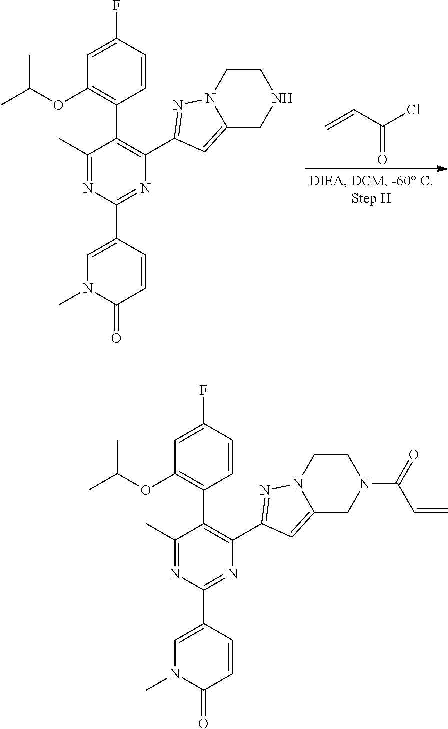

Step A: Preparation of 5-bromo-2-chloro-4-methoxy-6-methyl-pyrimidine: To a solution of 5-bromo-2,4-dichloro-6-methyl-pyrimidine (1.00 g, 4.13 mmol) in THF (10 mL) was added NaOMe (268 mg, 4.96 mmol), and the resulting mixture was stirred at 25° C. for 2 hours. The mixture was concentrated to give the crude product, which was purified by column chromatography on silica gel (petroleum ether/EtOAc: 3/1) to give 5-bromo-2-chloro-4-methoxy-6-methyl-pyrimidine (660 mg, 67% yield) as a white solid. LCMS ESI (+) m/z 237.0 (M+H).

Step B: Preparation of 5-(5-bromo-4-methoxy-6-methyl-pyrimidin-2-yl)-1-methyl-pyridin-2-one: To a solution of 5-bromo-2-chloro-4-methoxy-6-methyl-pyrimidine (650 mg, 2.74 mmol), 1-methyl-5-(4,4,5,5-tetramethyl-1,3,2-dioxaborolan-2-yl)pyridin-2-one (965 mg, 4.11 mmol) and Na₂CO₃ (580 mg, 5.47 mmol) in water (0.8 mL) and 1,4-dioxane (8 mL) was added Pd(PPh₃)₄(316 mg, 0.274 mmol) under nitrogen. The resulting mixture was stirred at 95° C. for 2 hours. The solvent was removed under reduced pressure. The residue was purified by column chromatography on silica gel (petroleum ether/EtOAc: 3/2) to afford 5-(5-bromo-4-methoxy-6-methyl-pyrimidin-2-yl)-1-methyl-pyridin-2-one (666 mg, 78% yield) as a white solid. LCMS ESI (+) m/z 310.1 (M+H).

Step C: Preparation of 5-[5-(4-fluoro-2-isopropoxy-phenyl)-4-methoxy-6-methyl-pyrimidin-2-yl]-1-methyl-pyridin-2-one: To a solution of (4-fluoro-2-isopropoxy-phenyl)boronic acid (383 mg, 1.93 mmol), 5-(5-bromo-4-methoxy-6-methyl-pyrimidin-2-yl)-1-methyl-pyridin-2-one (400 mg, 1.29 mmol) and Na₂CO₃ (273 mg, 2.58 mmol) in 1,4-dioxane (5 mL) and water (0.5 mL) was added Pd(PPh₃)₄(149 mg, 0.129 mmol) under nitrogen. The resulting mixture was stirred at 95° C. for 3 hours. The solvent was removed under reduced pressure. The residue was purified by column chromatography on silica gel (petroleum ether/EtOAc: 2/1) to afford 5-[5-(4-fluoro-2-isopropoxy-phenyl)-4-methoxy-6-methyl-pyrimidin-2-yl]-1-methyl-pyridin-2-one (410 mg, 83% yield) as a white solid. LCMS ESI (+) m/z 383.2 (M+H).

Step D: Preparation of 5-[5-(4-fluoro-2-isopropoxy-phenyl)-4-hydroxy-6-methyl-pyrimidin-2-yl]-1-methyl-pyridin-2-one: To a suspension of 5-[5-(4-fluoro-2-isopropoxy-phenyl)-4-methoxy-6-methyl-pyrimidin-2-yl]-1-methyl-pyridin-2-one (200 mg, 0.522 mmol) and NaI (777 mg, 5.22 mmol) in acetonitrile (6 mL) was added TMSCl (566 mg, 5.22 mmol) under nitrogen. The resulting mixture was stirred at 85° C. for 4 hours. The solvent was removed under reduced pressure. The residue was purified by Preparative TLC (DCM:MeOH=10:1) to afford 5-[5-(4-fluoro-2-isopropoxy-phenyl)-4-hydroxy-6-methyl-pyrimidin-2-yl]-1-methyl-pyridin-2-one (164 mg, 85% yield) as a yellow solid. LCMS ESI (+) m/z 370.2 (M+H).

Step E: Preparation of [5-(4-fluoro-2-isopropoxy-phenyl)-6-methyl-2-(1-methyl-6-oxo-3-pyridyl)pyrimidin-4-yl]trifluoromethanesulfonate: To a solution of 5-[5-(4-fluoro-2-isopropoxy-phenyl)-4-hydroxy-6-methyl-pyrimidin-2-yl]-1-methyl-pyridin-2-one (50 mg, 0.14 mmol) and pyridine (0.11 mL, 1.4 mmol) in DCM (2 mL) was added trifluoromethanesulfonic anhydride (0.034 mL, 0.20 mmol). The mixture was stirred at 0° C. for 1 hour. The solvent was removed under reduced pressure. The residue was purified by column chromatography on silica gel (petroleum ether/EtOAc=1:1) to afford [5-(4-fluoro-2-isopropoxy-phenyl)-6-methyl-2-(1-methyl-6-oxo-3-pyridyl)pyrimidin-4-yl]trifluoromethanesulfonate (44 mg, 65% yield) as a yellow solid. LCMS ESI (+) m/z 502.1 (M+H).

Step F: Preparation of tert-butyl 2-[5-(4-fluoro-2-isopropoxy-phenyl)-6-methyl-2-(1-methyl-6-oxo-3-pyridyl)pyrimidin-4-yl]-6,7-dihydro-4H-pyrazolo[1,5-a]pyrazine-5-carboxylate: To a solution of [5-(4-fluoro-2-isopropoxy-phenyl)-6-methyl-2-(1-methyl-6-oxo-3-pyridyl)pyrimidin-4-yl]trifluoromethane sulfonate (45 mg, 0.090 mmol), (5-tert-butoxycarbonyl-6,7-dihydro-4H-pyrazolo[1,5-a]pyrazin-2-yl)boronic acid (36 mg, 0.14 mmol) and Na₂CO₃ (19 mg, 0.18 mmol) in 1,4-dioxane (2 mL) and water (0.2 mL) was added Pd(PPh₃)₄(10 mg, 0.0090 mmol) under nitrogen. The resulting mixture was stirred at 95° C. for 2 hours. The solvent was removed under reduced pressure. The residue was purified by Preparative TLC (DCM:MeOH=20:1) to afford tert-butyl 2-[5-(4-fluoro-2-isopropoxy-phenyl)-6-methyl-2-(1-methyl-6-oxo-3-pyridyl)pyrimidin-4-yl]-6,7-dihydro-4H-pyrazolo[1,5-a]pyrazine-5-carboxylate (37 mg, 72% yield) as a yellow solid. LCMS ESI (+) m/z 575.1 (M+H).

Step G: Preparation of 5-[5-(4-fluoro-2-isopropoxy-phenyl)-4-methyl-6-(4,5,6,7-tetrahydropyrazolo[1,5-a]pyrazin-2-yl)pyrimidin-2-yl]-1-methyl-pyridin-2-one: To a solution of tert-butyl 2-[5-(4-fluoro-2-isopropoxy-phenyl)-6-methyl-2-(1-methyl-6-oxo-3-pyridyl)pyrimidin-4-yl]-6,7-dihydro-4H-pyrazolo[1,5-a]pyrazine-5-carboxylate (37 mg, 0.064 mmol) in DCM (2 mL) was added trifluoroacetic acid (0.70 mL). The mixture was stirred at 20° C. for 1 hour. The resulting mixture was evaporated to dryness to afford 5-[5-(4-fluoro-2-isopropoxy-phenyl)-4-methyl-6-(4,5,6,7-tetrahydropyrazolo[1,5-a]pyrazin-2-yl)pyrimidin-2-yl]-1-methyl-pyridin-2-one trifluoroacetic acid salt (50 mg) as a yellow solid, which was used directly in the next step. LCMS ESI (+) m/z 475.0 (M+H).

Step H: Preparation of 5-[5-(4-fluoro-2-isopropoxy-phenyl)-4-methyl-6-(5-prop-2-enoyl-6,7-dihydro-4H-pyrazolo[1,5-a]pyrazin-2-yl)pyrimidin-2-yl]-1-methyl-pyridin-2-one: To a solution of 5-[5-(4-fluoro-2-isopropoxy-phenyl)-4-methyl-6-(4,5,6,7-tetrahydropyrazolo[1,5-a]pyrazin-2-yl)pyrimidin-2-yl]-1-methyl-pyridin-2-one (37 mg, 0.078 mmol) and DIEA (0.14 mL, 0.78 mmol) in DCM (2 mL) was added acryloyl chloride (0.0095 mL, 0.12 mmol) at −60° C., the resulting mixture was stirred at −60° C. for 1 hour. The solvent was poured into NaHCO₃ aqueous solution and the product was extracted with DCM. The organic phase was dried over anhydrous Na₂SO₄, filtered and concentrated. The residue was purified by Preparative HPLC to afford 5-[5-(4-fluoro-2-isopropoxy-phenyl)-4-methyl-6-(5-prop-2-enoyl-6,7-dihydro-4H-pyrazolo[1,5-a]pyrazin-2-yl)pyrimidin-2-yl]-1-methyl-pyridin-2-one (12 mg, 27% yield) as a trifluoroacetic acid salt. ¹H NMR (400 MHz, CD₃OD) δ 9.02 (d, J=1.8 Hz, 1H), 8.64 (dd, J=1.8, 9.6 Hz, 1H), 7.09 (t, J=7.7 Hz, 1H), 6.71-6.93 (m, 3H), 6.65 (d, J=9.5 Hz, 1H), 6.27 (d, J=17.0 Hz, 1H), 5.77-5.85 (m, 1H), 5.75 (s, 1H), 4.68-4.84 (m, 2H), 4.47-4.59 (m, 1H), 4.21 (s, 2H), 4.06-4.15 (m, 2H), 3.72 (s, 3H), 2.32 (s, 3H), 1.17 (d, J=5.9 Hz, 3H), 0.95 (d, J=5.9 Hz, 3H). LCMS ESI (+) m/z 529.3 (M+H).

### Synthetic Example 49: Synthesis of 5-[7-(2,4-difluoro-6-isopropoxy-phenyl)-6-[1-[(3S)-1-prop-2-enoylpyrrolidin-3-yl]pyrazol-4-yl]thieno[3,2-c]pyridin-4-yl]isoindolin-1-one (Compound 334)

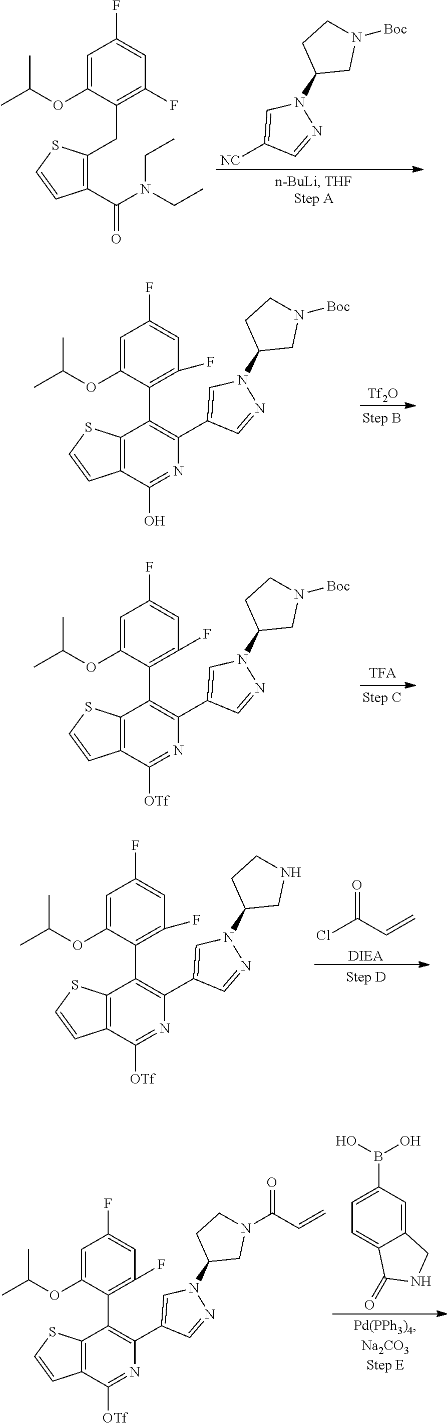

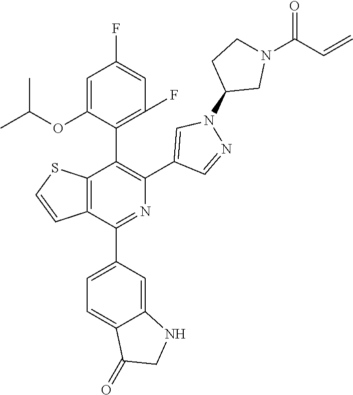 [SMILES: C=CC(=O)N1CC[C@H](n2cc(-c3nc(-c4ccc5c(c4)NCC5=O)c4ccsc4c3-c3c(F)cc(F)cc3OC(C)C)cn2)C1]

Step A: Preparation of tert-butyl (3S)-3-(4-(7-(2,4-difluoro-6-isopropoxyphenyl)-4-hydroxythieno[3,2-c]pyridin-6-yl)-1H-pyrazol-1-yl)pyrrolidine-1-carboxylate: To a solution of 2-(2,4-difluoro-6-isopropoxybenzyl)-N,N-diethylthiophene-3-carboxamide (200 mg, 0.545 mmol) in dry THF (2 mL) was added n-BuLi (0.24 mL, 2.5 M in hexanes, 0.60 mmol) at −60° C. The mixture was stirred at −60° C. for 20 min. A solution of tert-butyl (S)-3-(4-cyano-1H-pyrazol-1-yl)pyrrolidine-1-carboxylate (157 mg, 0.600 mmol) in THF was added. The resulting mixture was stirred at 25° C. for 2 h. Saturated sodium bicarbonate solution was added to quench the reaction. The mixture was extracted with EtOAc, washed with brine, dried over Na₂SO₄, filtered and concentrated. The residue was purified by Preparative TLC (petroleum ether/EtOAc=1/1) to give tert-butyl (3S)-3-(4-(7-(2,4-difluoro-6-isopropoxyphenyl)-4-hydroxythieno[3,2-c]pyridin-6-yl)-1H-pyrazol-1-yl)pyrrolidine-1-carboxylate (180 mg, 60% yield). LCMS ESI (+) m/z 557.0 (M+H).

Step B: Preparation of tert-butyl (3S)-3-(4-(7-(2,4-difluoro-6-isopropoxyphenyl)-4-(((trifluoromethyl) sulfonyl)oxy)thieno[3,2-c]pyridin-6-yl)-1H-pyrazol-1-yl)pyrrolidine-1-carboxylate: To a solution of tert-butyl (3S)-3-(4-(7-(2,4-difluoro-6-isopropoxyphenyl)-4-hydroxythieno[3,2-c]pyridin-6-yl)-1H-pyrazol-1-yl)pyrrolidine-1-carboxylate (130 mg, 0.230 mmol) and pyridine (185 mg, 2.30 mmol) in DCM (3 mL) was added trifluoromethanesulfonic anhydride (100 mg, 0.350 mmol). The mixture was stirred at 0° C. for 0.5 h. The mixture was diluted with water and extracted with DCM. The organic phase was separated, dried over Na₂SO₄, filtered and concentrated. The residue was purified by Preparative TLC (EtOAc/petroleum ether: 1/5) to give tert-butyl (3S)-3-(4-(7-(2,4-difluoro-6-isopropoxyphenyl)-4-(((trifluoromethyl)sulfonyl)oxy)thieno[3,2-c]pyridin-6-yl)-1H-pyrazol-1-yl)pyrrolidine-1-carboxylate (140 mg, 87% yield).

Step C: Preparation of 7-(2,4-difluoro-6-isopropoxyphenyl)-6-(1-((S)-pyrrolidin-3-yl)-1H-pyrazol-4-yl)thieno[3,2-c]pyridin-4-yl trifluoromethanesulfonate: A mixture of tert-butyl (3S)-3-(4-(7-(2,4-difluoro-6-isopropoxyphenyl)-4-(((trifluoromethyl)sulfonyl)oxy)thieno[3,2-c]pyridin-6-yl)-1H-pyrazol-1-yl)pyrrolidine-1-carboxylate (70 mg, 0.10 mmol) and TFA (0.3 mL) in DCM (2 mL) was stirred at 25° C. for 0.5 h. The solvent was removed in vacuo to give crude 7-(2,4-difluoro-6-isopropoxyphenyl)-6-(1-((S)-pyrrolidin-3-yl)-1H-pyrazol-4-yl)thieno[3,2-c]pyridin-4-yl trifluoromethanesulfonate (80 mg) as a trifluoroacetic acid salt which was used in the next step without further purification. LCMS ESI (+) m/z 588.9 (M+H).

Step D: Preparation of 6-(1-((S)-1-acryloylpyrrolidin-3-yl)-1H-pyrazol-4-yl)-7-(2,4-difluoro-6-isopropoxyphenyl)thieno[3,2-c]pyridin-4-yl trifluoromethanesulfonate: To a solution of 7-(2,4-difluoro-6-isopropoxyphenyl)-6-(1-((S)-pyrrolidin-3-yl)-1H-pyrazol-4-yl)thieno[3,2-c]pyridin-4-yl trifluoromethanesulfonate (80 mg, crude) and DIEA (0.18 mL, 1.3 mmol) in DCM (2 mL) was added acryloyl chloride (0.016 mL, 0.19 mmol) at −60° C. The mixture was stirred at −60° C. for 0.5 h. Saturated sodium bicarbonate solution was added to quench the reaction. The mixture was extracted with EtOAc, washed with brine, dried (Na₂SO₄), filtered and concentrated. The crude was purified by Preparative TLC (DCM/MeOH=20/1) to give 6-(1-((S)-1-acryloylpyrrolidin-3-yl)-1H-pyrazol-4-yl)-7-(2,4-difluoro-6-isopropoxyphenyl)thieno[3,2-c]pyridin-4-yl trifluoromethanesulfonate (60 mg, 93% yield). LCMS ESI (+) m/z 642.9 (M+H).

Step E: Preparation of 5-[7-(2,4-difluoro-6-isopropoxy-phenyl)-6-[1-[(3S)-1-prop-2-enoylpyrrolidin-3-yl]pyrazol-4-yl]thieno[3,2-c]pyridin-4-yl]isoindolin-1-one: A mixture of [7-(2,4-difluoro-6-isopropoxy-phenyl)-6-[1-[(3S)-1-prop-2-enoylpyrrolidin-3-yl]pyrazol-4-yl]thieno[3,2-c]pyridin-4-yl]trifluoromethanesulfonate (20 mg, 0.031 mmol), (1-oxoisoindolin-5-yl)boronic acid (8.3 mg, 0.047 mmol), sodium carbonate (6.6 mg, 0.062 mmol) and tetrakis(triphenylphosphine)palladium (3.6 mg, 0.0031 mmol) in 1,4-dioxane (0.50 mL)/water (0.10 mL) was stirred at 100° C. under argon for 2 h. The reaction was filtered and concentrated. The residue was taken up in ACN (0.5 mL), and purified by Preparative HPLC to give 5-[7-(2,4-difluoro-6-isopropoxy-phenyl)-6-[1-[(3S)-1-prop-2-enoylpyrrolidin-3-yl]pyrazol-4-yl]thieno[3,2-c]pyridin-4-yl]isoindolin-1-one (9.1 mg, 47% yield) as a trifluoroacetic acid salt. ¹H NMR (400 MHz, CD₃OD) δ 8.23 (s, 1H), 8.15 (d, J=8.0 Hz, 1H), 8.08 (d, J=8.0 Hz, 1H), 7.83-7.95 (m, 2H), 7.72-7.79 (m, 1H), 7.52-7.60 (m, 1H), 6.89-6.98 (m, 1H), 6.80 (t, J=9.2 Hz, 1H), 6.53-6.68 (m, 1H), 6.32 (dd, J=6.4, 16.8 Hz, 1H), 5.79 (t, J=10.2 Hz, 1H), 5.04-5.13 (m, 1H), 4.58-4.72 (m, 3H), 3.95-4.14 (m, 1H), 3.88-3.95 (m, 1H), 3.79-3.86 (m, 1H), 3.61-3.73 (m, 1H), 2.33-2.57 (m, 2H), 1.14 (d, J=6.0 Hz, 3H), 0.95-1.05 (m, 3H). LCMS ESI (+) m/z 626.0 (M+H).

### Synthetic Example 50: Synthesis of 5-[5-(4-fluoro-2-isopropoxy-phenyl)-4-(5-prop-2-enoyl-6,7-dihydro-4H-pyrazolo [1,5-a]pyrazin-2-yl)pyrimidin-2-yl]-1-methyl-pyridin-2-one (Compound 337)

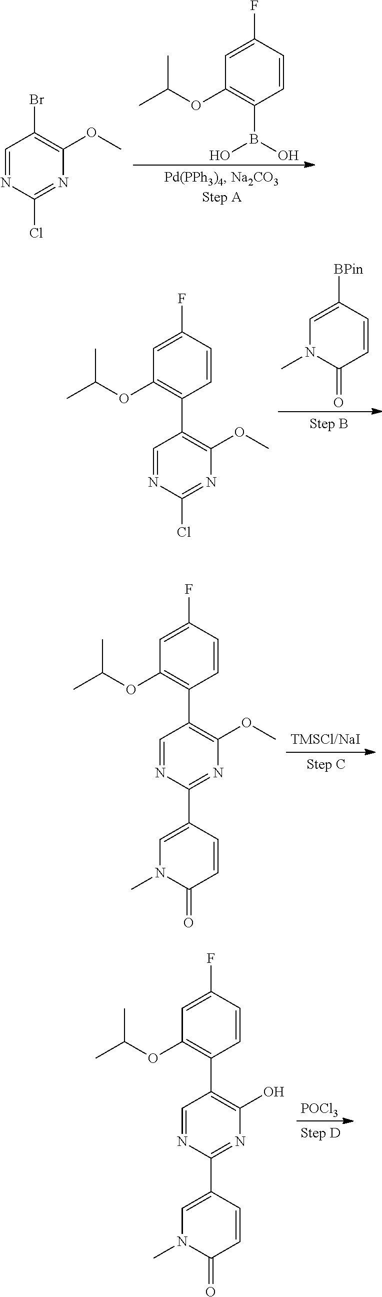

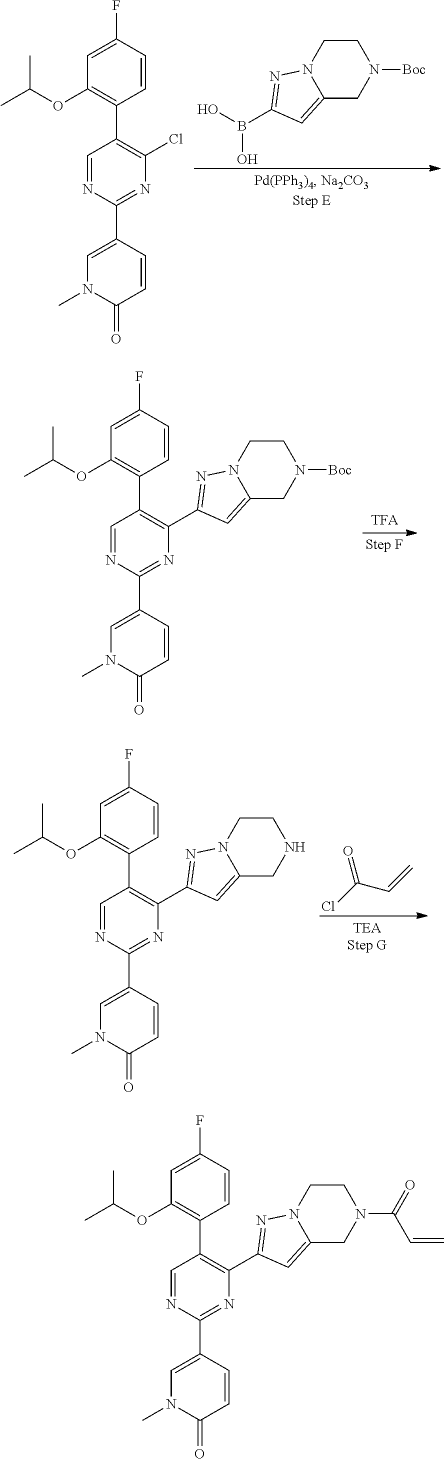

Step A: Preparation 2-chloro-5-(4-fluoro-2-isopropoxy-phenyl)-4-methoxy-pyrimidine: To a stirred solution of 5-bromo-2-chloro-4-methoxy-pyrimidine (1.00 g, 4.48 mmol) in 1,4-dioxane (5 mL)/water (0.5 mL) were added Na₂CO₃ (1.40 g, 13.2 mmol) and Pd(PPh₃)₄(520 mg, 0.450 mmol). The mixture was stirred at 80° C. for 2 h under Argon. The mixture was concentrated and purified by column chromatography (petroleum ether:EtOAc=100:1 to 3:1) to give 2-chloro-5-(4-fluoro-2-isopropoxy-phenyl)-4-methoxy-pyrimidine (550 mg, 46% yield) as a white solid. LCMS ESI (+) m/z 297.3 (M+H).

Step B: Preparation of 5-[5-(4-fluoro-2-isopropoxy-phenyl)-4-methoxy-pyrimidin-2-yl]-1-methyl-pyridin-2-one: To a stirred solution of 2-chloro-5-(4-fluoro-2-isopropoxy-phenyl)-4-methoxy-pyrimidine (170 mg, 0.573 mmol) in 1,4-dioxane (5 mL) and water (0.5 mL) were added Na₂CO₃ (66 mg, 0.62 mmol) and Pd(PPh₃)₄(183 mg, 0.158 mmol). The mixture was stirred at 80° C. for 2 h under Argon. The mixture was then concentrated and purified by column chromatography (petroleum ether:EtOAc=5:1) to give 5-[5-(4-fluoro-2-isopropoxy-phenyl)-4-methoxy-pyrimidin-2-yl]-1-methyl-pyridin-2-one (178 mg, 90% yield) as a white solid. LCMS ESI (+) m/z 370.2 (M+H).

Step C: Preparation of 5-[5-(4-fluoro-2-isopropoxy-phenyl)-4-hydroxy-pyrimidin-2-yl]-1-methyl-pyridin-2-one: To a stirred solution of 5-[5-(4-fluoro-2-isopropoxy-phenyl)-4-methoxy-pyrimidin-2-yl]-1-methyl-pyridin-2-one (170 mg, 0.460 mmol) in MeCN (8 mL) were added trimethylchlorosilane (0.29 mL, 2.30 mmol) and NaI (345 mg, 2.30 mmol). The solution was stirred at 8° C. for 3 h, then stirred at 50° C. for 12 h. Sodium sulfite aqueous solution was added to quench the reaction. The reaction was diluted with water and extracted with ethyl acetate. The combined organic layers were washed with saturated brine solution, dried over Na₂SO₄, filtered and concentrated. The residue was purified by Preparative TLC (DCM:MeOH=20:1) to give 5-[5-(4-fluoro-2-isopropoxy-phenyl)-4-hydroxy-pyrimidin-2-yl]-1-methyl-pyridin-2-one (123 mg, 75% yield) as a white solid. LCMS ESI (+) m/z 356.1 (M+H).

Step D: Preparation of tert-butyl 2-[5-(4-fluoro-2-isopropoxy-phenyl)-2-(1-methyl-6-oxo-3-pyridyl)pyrimidin-4-yl]-6,7-dihydro-4H-pyrazolo[1,5-a]pyrazine-5-carboxylate: A solution of 5-[5-(4-fluoro-2-isopropoxy-phenyl)-4-hydroxy-pyrimidin-2-yl]-1-methyl-pyridin-2-one (130 mg, 0.366 mmol) in phosphorus oxychloride (2.00 mL, 21.5 mmol) was stirred at 24° C. for 5 h. The reaction solution was poured into ice-water, and saturated NaHCO₃ aqueous solution was added to adjust the pH to 8. The aqueous layer was extracted with ethyl acetate. The combined organic layers were washed with saturated brine, dried over Na₂SO₄, filtered and concentrated. The residue was purified by Preparative TLC (petroleum ether/EtOAc=1:1) to give tert-butyl 2-[5-(4-fluoro-2-isopropoxy-phenyl)-2-(1-methyl-6-oxo-3-pyridyl)pyrimidin-4-yl]-6,7-dihydro-4H-pyrazolo[1,5-a]pyrazine-5-carboxylate (17 mg, 12% yield) as a white solid. LCMS ESI (+) m/z 374.2 (M+H).

Step E: Preparation of tert-butyl 2-[5-(4-fluoro-2-isopropoxy-phenyl)-2-(1-methyl-6-oxo-3-pyridyl)pyrimidin-4-yl]-6,7-dihydro-4H-pyrazolo[1,5-a]pyrazine-5-carboxylate: To a solution of 5-[4-chloro-5-(4-fluoro-2-isopropoxy-phenyl)pyrmidin-2-yl]-1-methyl-pyridin-2-one (12 mg, 0.032 mmol) in 1,4-dioxane (5 mL) and water (0.1 mL) were added Na₂CO₃ (9.9 mg, 0.093 mmol) and Pd(PPh₃)₄ (5.4 mg, 0.0047 mmol). The mixture was stirred at 80° C. for 2 h under Argon. The mixture was concentrated and purified by Preparative TLC (EtOAc) to give tert-butyl 2-[5-(4-fluoro-2-isopropoxy-phenyl)-2-(1-methyl-6-oxo-3-pyridyl)pyrimidin-4-yl]-6,7-dihydro-4H-pyrazolo[1,5-a]pyrazine-5-carboxylate (12 mg, 67% yield) as a white solid. LCMS ESI (+) m/z 561.1 (M+H).

Step F: Preparation of 5-[5-(4-fluoro-2-isopropoxy-phenyl)-4-(4,5,6,7-tetrahydropyrazolo[1,5-a]pyrazin-2-yl)pyrimidin-2-yl]-1-methyl-pyridin-2-one: To a stirred solution of tert-butyl 2-[5-(4-fluoro-2-isopropoxy-phenyl)-2-(1-methyl-6-oxo-3-pyridyl)pyrimidin-4-yl]-6,7-dihydro-4H-pyrazolo[1,5-a]pyrazine-5-carboxylate (12 mg, 0.020 mmol) in DCM (1.5 mL) was added trifluoroacetic acid (0.50 mL, 6.5 mmol). The solution was stirred at 10° C. for 1 h. The mixture was concentrated to give 5-[5-(4-fluoro-2-isopropoxy-phenyl)-4-(4,5,6,7-tetrahydropyrazolo[1,5-a]pyrazin-2-yl)pyrimidin-2-yl]-1-methyl-pyridin-2-one trifluoroacetic acid (16 mg, 100% yield) which was used in the next step directly.

Step G: Preparation of 5-[5-(4-fluoro-2-isopropoxy-phenyl)-4-(5-prop-2-enoyl-6,7-dihydro-4H-pyrazolo[1,5-a]pyrazin-2-yl)pyrimidin-2-yl]-1-methyl-pyridin-2-one: To a stirred solution of 5-[5-(4-fluoro-2-isopropoxy-phenyl)-4-(4,5,6,7-tetrahydropyrazolo[1,5-a]pyrazin-2-yl)pyrimidin-2-yl]-1-methyl-pyridin-2-one (14 mg, 0.030 mmol) and triethylamine (0,042 mL, 0.30 mmol) in DCM (1.5 mL) at −20° C. was added acryloyl chloride (0.0049 mL, 0.061 mmol). The solution was stirred at −20° C. for 20 min. The reaction was diluted with aqueous NaHCO₃ solution and extracted with EtOAc. The organic phase was dried over Na₂SO₄, filtered and concentrated. The residue was purified by Preparative HPLC to give 5-[5-(4-fluoro-2-isopropoxy-phenyl)-4-(5-prop-2-enoyl-6,7-dihydro-4H-pyrazolo[1,5-a]pyrazin-2-yl)pyrimidin-2-yl]-1-methyl-pyridin-2-one (7.4 mg, 46% yield) as a white solid. ¹H NMR (400 MHz, CD₃OD) δ 9.01 (d, J=2.0 Hz, 1H), 8.61 (dd, J=9.4, 2.2 Hz, 1H), 8.55 (s, 1H), 7.21-7.35 (m, 1H), 6.70-6.93 (m, 3H), 6.65 (d, J=9.4 Hz, 1H), 6.28 (d, J=16.0 Hz, 1H), 6.05 (s, 1H), 5.82 (d, J=8.0 Hz, 1H), 4.77 (s, 1H), 4.37-4.50 (m, 1H), 4.21 (s, 2H), 3.96-4.19 (m, 3H), 3.71 (s, 3H), 0.97 (s, 6H). LCMS ESI (+) m/z 515.1 (M+H).

### Synthetic Example 51: Synthesis of 1-[2-[5-chloro-3-[4-fluoro-2-(2-methoxyethoxy)phenyl]-6-(1-methylpyrazol-4-yl)-2-pyridyl]-6,7-dihydro-4H-pyrazolo[1,5-a]pyrazin-5-yl]prop-2-en-1-one (Compound 355)

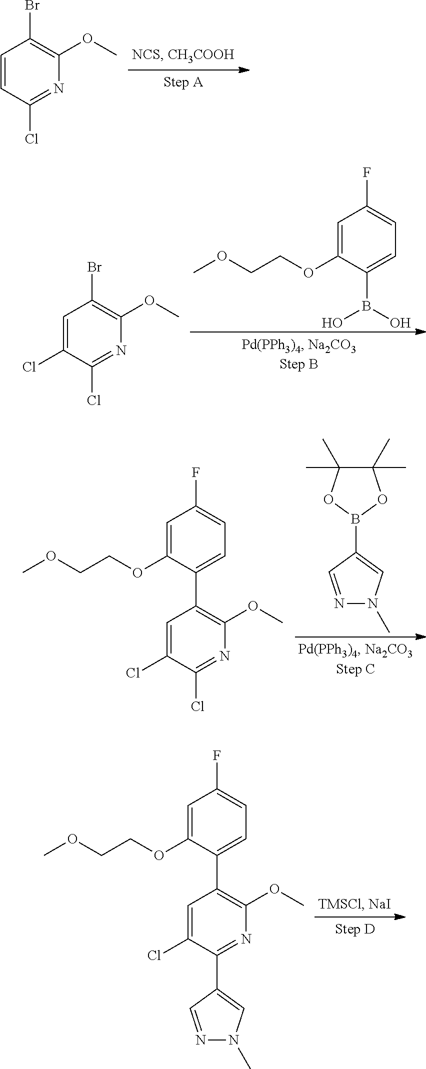

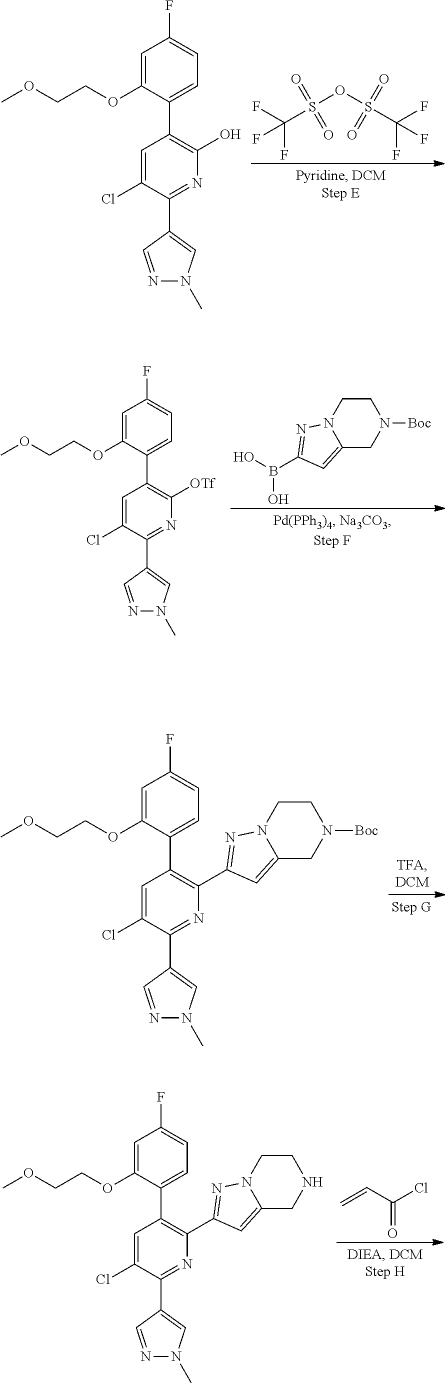

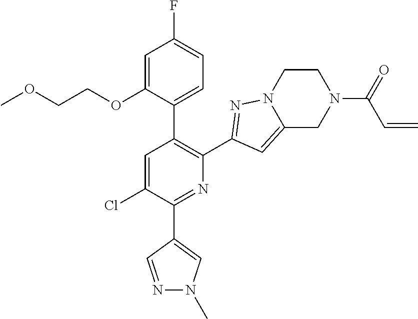 [SMILES: C=CC(=O)N1CCn2nc(-c3nc(-c4cnn(C)c4)c(Cl)cc3-c3ccc(F)cc3OCCOC)cc2C1]

Step A: Preparation of 5-bromo-2,3-dichloro-6-methoxy-pyridine: A solution of 3-bromo-6-chloro-2-methoxy-pyridine (500 mg, 2.25 mmol) and NCS (330 mg, 2.47 mmol) in acetic acid (10 mL) was stirred at 110° C. for 16 h. The mixture was concentrated. The crude was purified by column chromatography (petroleum ether/EtOAc=6/1) to give 5-bromo-2,3-dichloro-6-methoxy-pyridine (421 mg, 73% yield). LCMS ESI (+) m/z 258.0 (M+H).

Step B: Preparation of 2,3-dichloro-5-[4-fluoro-2-(2-methoxyethoxy)phenyl]-6-methoxy-pyridine: A mixture of [4-fluoro-2-(2-methoxyethoxy)phenyl]boronic acid (108 mg, 0.506 mmol), 5-bromo-2,3-dichloro-6-methoxy-pyridine (100 mg, 0.389 mmol), Pd(PPh₃)₄(45 mg, 0.039 mmol) and Na₂CO₃ (83 mg, 0.78 mmol) in 1,4-dioxane (1 mL) and water (0.10 mL) was stirred at 60° C. for 5 h under Ar. The mixture was concentrated and purified by Preparative TLC (petroleum ether/EtOAc=5/1) to give 2,3-dichloro-5-[4-fluoro-2-(2-methoxyethoxy)phenyl]-6-methoxy-pyridine (33 mg, 25% yield). LCMS ESI (+) m/z 346.0 (M+H).

Step C: Preparation of 5-chloro-3-[4-fluoro-2-(2-methoxyethoxy)phenyl]-2-methoxy-6-(1-methylpyrazol-4-yl)pyridine: A mixture of 2,3-dichloro-5-[4-fluoro-2-(2-methoxyethoxy)phenyl]-6-methoxy-pyridine (42 mg, 0.12 mmol), 1-methyl-4-(4,4,5,5-tetramethyl-1,3,2-dioxaborolan-2-yl)pyrazole (50 mg, 0.24 mmol), Pd(PPh₃)₄(8.9 mg, 0.012 mmol) and Na₂CO₃ (26 mg, 0.24 mmol) in 1,4-dioxane (2 mL) and water (0.2 mL) was stirred at 100° C. for 16 h. The mixture was concentrated and purified by Preparative TLC (petroleum ether/EtOAc=1/1) to give 5-chloro-3-[4-fluoro-2-(2-methoxyethoxy)phenyl]-2-methoxy-6-(1-methylpyrazol-4-yl)pyridine (40 mg, 84% yield). LCMS ESI (+) m/z 392.2 (M+H).

Step D: Preparation of 5-chloro-3-[4-fluoro-2-(2-methoxyethoxy)phenyl]-6-(1-methylpyrazol-4-yl)pyridin-2-ol: A mixture of 5-chloro-3-[4-fluoro-2-(2-methoxyethoxy)phenyl]-2-methoxy-6-(1-methylpyrazol-4-yl)pyridine (46 mg, 0.12 mmol), TMSCl (127 mg, 1.17 mmol), and NaI (53 mg, 0.35 mmol) in acetonitrile (2 mL) was stirred at 30° C. for 3 h. NaHSO₃ solution was added to quench the reaction. The product was extracted with DCM, dried over anhydrous Na₂SO₄, filtered and concentrated. The residue was purified by Preparative TLC (DCM/MeOH=20/1) to give 5-chloro-3-[4-fluoro-2-(2-methoxyethoxy)phenyl]-6-(1-methylpyrazol-4-yl)pyridin-2-ol (23 mg, 52% yield). LCMS ESI (+) m/z 378.2 (M+H).

Step E: Preparation of [5-chloro-3-[4-fluoro-2-(2-methoxyethoxy)phenyl]-6-(1-methylpyrazol-4-yl)-2-pyridyl]trifluoromethanesulfonate: To a stirred solution of 5-chloro-3-[4-fluoro-2-(2-methoxyethoxy)phenyl]-6-(1-methylpyrazol-4-yl)pyridin-2-ol (33 mg, 0.087 mmol) and pyridine (35 mg, 0.44 mmol) in CH₃CN (2 mL) was added trifluoromethanesulfonic anhydride (0.030 mL, 0.18 mmol) at 0° C. The mixture was stirred at 25° C. for 1 hour. Water was added to quench the reaction, and the mixture was extracted with EtOAc. The organic phase was dried over Na₂SO₄, filtered and concentrated. The residue was purified by Preparative TLC (DCM/MeOH=20/1) to give [5-chloro-3-[4-fluoro-2-(2-methoxyethoxy)phenyl]-6-(1-methylpyrazol-4-yl)-2-pyridyl]trifluoromethanesulfonate (22 mg, 50% yield). LCMS ESI (+) m/z 510.1 (M+H).

Step F: Preparation of tert-butyl 2-[5-chloro-3-[4-fluoro-2-(2-methoxyethoxy)phenyl]-6-(1-methylpyrazol-4-yl)-2-pyridyl]-6,7-dihydro-4H-pyrazolo[1,5-a]pyrazine-5-carboxylate: A mixture of [5-chloro-3-[4-fluoro-2-(2-methoxyethoxy)phenyl]-6-(1-methylpyrazol-4-yl)-2-pyridyl]trifluoromethanesulfonate (17 mg, 0.033 mmol), (5-tert-butoxycarbonyl-6,7-dihydro-4H-pyrazolo[1,5-a]pyrazin-2-yl)boronic acid (31 mg, 0.12 mmol), Pd(PPh₃)₄(2.4 mg, 0.0033 mmol) and Na₂CO₃ (7.1 mg, 0.067 mmol) in 1,4-dioxane (2 mL)/water (0.2 mL) was stirred at 95° C. for 2 h. The mixture was concentrated and purified by Preparative TLC (DCM/MeOH=20/1) to give tert-butyl 2-[5-chloro-3-[4-fluoro-2-(2-methoxyethoxy)phenyl]-6-(1-methylpyrazol-4-yl)-2-pyridyl]-6,7-dihydro-4H-pyrazolo[1,5-a]pyrazine-5-carboxylate (14 mg, 72% yield). LCMS ESI (+) m/z 583.3 (M+H).

Step G: Preparation of 2-[5-chloro-3-[4-fluoro-2-(2-methoxyethoxy)phenyl]-6-(1-methylpyrazol-4-yl)-2-pyridyl]-4,5,6,7-tetrahydropyrazolo[1,5-a]pyrazine: To a solution of tert-butyl 2-[5-chloro-3-[4-fluoro-2-(2-methoxyethoxy)phenyl]-6-(1-methylpyrazol-4-yl)-2-pyridyl]-6,7-dihydro-4H-pyrazolo[1,5-a]pyrazine-5-carboxylate (14 mg, 0.024 mmol) in DCM (2 mL) was added TFA (0.50 mL). The mixture was stirred at 25° C. for 0.5 h. The mixture was concentrated in vacuo to dryness to give crude 2-[5-chloro-3-[4-fluoro-2-(2-methoxvethoxy)phenyl]-6-(1-methylpyrazol-4-yl)-2-pyridyl]-4,5,6,7-tetrahydropyrazolo[1,5-a]pyrazine which was used in the next step directly.

Step H: Preparation of 1-[2-[5-chloro-3-[4-fluoro-2-(2-methoxyethoxy)phenyl]-6-(1-methylpyrazol-4-yl)-2-pyridyl]-6,7-dihydro-4H-pyrazolo[1,5-a]pyrazin-5-yl]prop-2-en-1-one: To a stirred solution of 2-[5-chloro-3-[4-fluoro-2-(2-methoxyethoxy)phenyl]-6-(1-methylpyrazol-4-yl)-2-pyridyl]-4,5,6,7-tetrahydropyrazolo[1,5-a]pyrazine (25 mg, 0.052 mmol) and DIPEA (0.50 mL, 2.8 mmol) in DCM (2 mL) was added acryloyl chloride (0.0050 mL, 0.062 mmol) at 0° C. The mixture was stirred at 25° C. for 0.5 h. The mixture was poured into NaHCO₃ solution and extracted with DCM. The combined organic layers were dried over Na₂SO₄, filtered and concentrated. The residue was purified by Preparative HPLC to give 1-[2-[5-chloro-3-[4-fluoro-2-(2-methoxyethoxy)phenyl]-6-(1-methylpyrazol-4-yl)-2-pyridyl]-6,7-dihydro-4H-pyrazolo[1,5-a]pyrazin-5-yl]prop-2-en-1-one (2.0 mg, 7% yield) as a bis trifluoroacetic acid salt. ¹H NMR (400 MHz, CD₃OD) δ 8.49 (s, 1H), 8.34 (s, 1H), 7.76 (s, 1H), 7.17 (t, J=7.6 Hz, 1H), 6.79-6.90 (m, 2H), 6.72-6.77 (m, 1H), 6.27 (d, J=16.7 Hz, 1H), 5.92 (s, 1H), 5.81 (d, J=10.5 Hz, 1H), 4.71-4.80 (m, 2H), 4.13-4.23 (m, 2H), 4.06-4.12 (m, 2H), 3.99 (s, 3H), 3.88-3.95 (m, 2H), 3.46 (t, J=4.7 Hz, 2H), 3.22 (s, 3H). LCMS ESI (+) m/z 537.2 (M+H).

### Synthetic Example 52: Synthesis of 1-((7R)-2-(7-(4-fluoro-2-isopropoxyphenyl)-4-(1-methyl-1H-pyrazol-4-yl)thieno[3,2-c]pyridin-6-yl)-7-methyl-6,7-dihydropyrazolo[1,5-a]pyrazin-5(4H)-yl)prop-2-en-1-one (Compound 357)

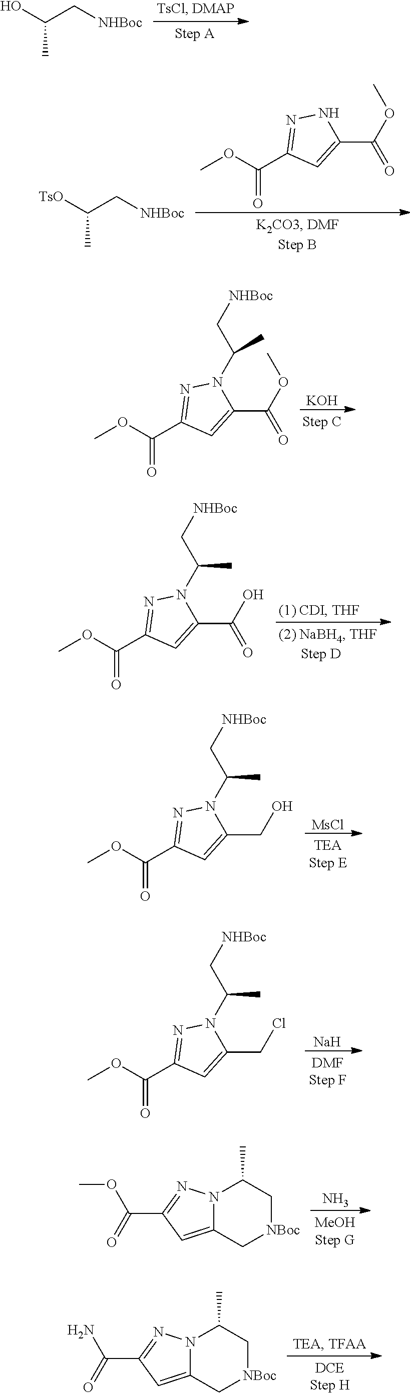

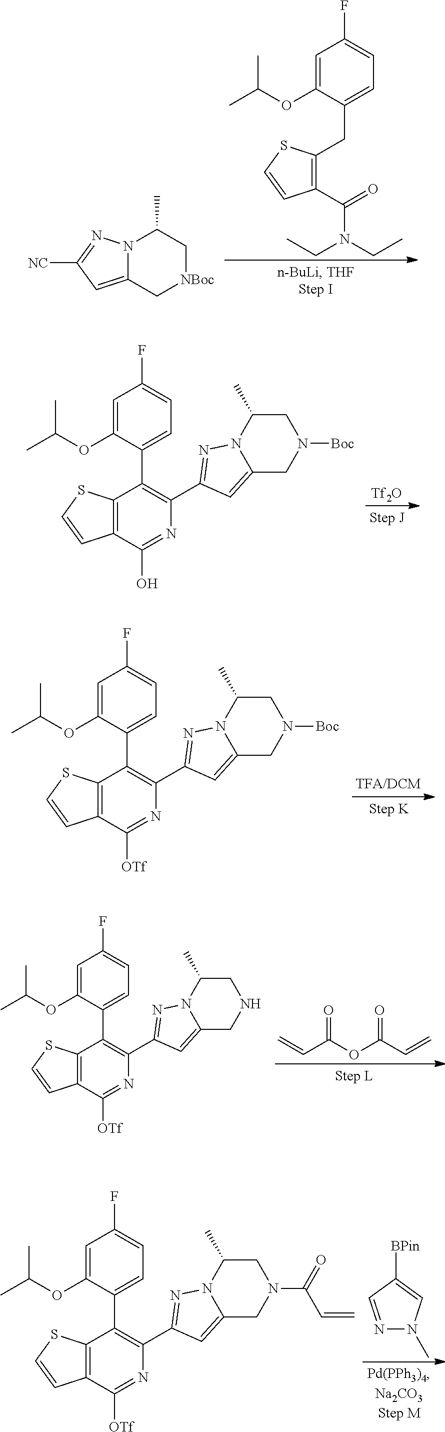

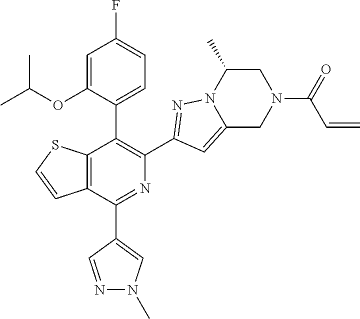 [SMILES: C=CC(=O)N1Cc2cc(-c3nc(-c4cnn(C)c4)c4ccsc4c3-c3ccc(F)cc3OC(C)C)nn2[C@H](C)C1]

Step A: Preparation of [(1S)-2-(tert-butoxycarbonylamino)-1-methyl-ethyl]4-methylbenzenesulfonate: To a solution of tert-butyl N-[(2S)-2-hydroxypropyl]carbamate (3.00 g, 17.1 mmol) in DCM (30 mL) was added triethylamine (7.20 mL, 51.4 mmol) and DMAP (209 mg, 1.71 mmol), followed by p-toluenesulfonyl chloride (4.24 g, 22.3 mmol) at 0° C. The mixture was stirred at 25° C. for 10 h. The mixture was poured into NaHCO₃ aqueous solution. The product was extracted with EtOAc, dried over anhydrous Na₂SO₄, concentrated and purified by column chromatography (petroleum ether/EtOAc=20/1 to 3/1) to give [(1S)-2-(tert-butoxycarbonylamino)-1-methyl-ethyl]4-methylbenzenesulfonate (3.90 g, 69% yield) as a white solid. LCMS ESI (+) m/z 230.1 (M+H).

Step B: Preparation of dimethyl (R)-1-(1-((tert-butoxycarbonyl)amino)propan-2-yl)-1H-pyrazole-3,5-dicarboxylate: To a solution of dimethyl 1H-pyrazole-3,5-dicarboxylate (1.63 g, 8.85 mmol) and [(1S)-2-(tert-butoxycarbonylamino)-1-methyl-ethyl]4-methylbenzenesulfonate (3.50 g, 10.6 mmol) in DMF (20 mL) was added potassium carbonate (1.84 g, 13.3 mmol). The mixture was stirred at 25° C. for 12 h under Ar. The mixture was poured into water. The product was extracted with EtOAc, dried over anhydrous Na₂SO₄, concentrated and purified by column chromatography (petroleum ether/EtOAc=30/1 to 5/1) to give dimethyl (R)-1-(1-((tert-butoxycarbonyl)amino)propan-2-yl)-1H-pyrazole-3,5-dicarboxylate (1.80 g, 60% yield). LCMS ESI (+) m/z 242.1 (M+H-Boc).

Step C: Preparation of 2-[(1R)-2-(tert-butoxycarbonylamino)-1-methyl-ethyl]-5-methoxycarbonyl-pyrazole-3-carboxylic acid: To a solution of dimethyl 1-[(1R)-2-(tert-butoxycarbonylamino)-1-methyl-ethyl]pyrazole-3,5-dicarboxylate (1.90 g, 5.57 mmol) in DCM (10 mL) and methanol (10 mL) was added potassium hydroxide (0.340 g, 2.0 M solution in methanol, 6.12 mmol). The mixture was stirred at 25° C. for 4 h. The reaction solution was concentrated and diluted with water. The mixture was extracted with MTBE. 1 N aqueous HCl was added to adjust the pH to 3. The mixture was extracted with EtOAc, dried over anhydrous Na₂SO₄, and then concentrated in vacuo to yield crude 2-[(1R)-2-(tert-butoxycarbonylamino)-1-methyl-ethyl]-5-methoxycarbonyl-pyrazole-3-carboxylic acid (1.10 g, 60% yield). LCMS ESI (−) m/z 326.0 (M−H).

Step D: Preparation of methyl 1-[(1R)-2-(tert-butoxycarbonylamino)-1-methyl-ethyl]-5-(hydroxymethyl)pyrazole-3-carboxylate: To a solution of 2-[(1R)-2-(tert-butoxycarbonylamino)-1-methyl-ethyl]-5-methoxycarbonyl-pyrazole-3-carboxylic acid (1.00 g, 3.06 mmol) in THF (10 mL) was added N,N′-carbonyldiimidazole (0.640 g, 3.97 mmol). The mixture was stirred at 45° C. for 1 h under Ar. To the reaction solution, aqueous 2.0 M solution NaBH₄ in THF (0.460 g, 12.2 mmol) was added at 0° C. The mixture was then stirred at room temperature for 1 h. Aqueous saturated ammonium chloride solution was added, and the mixture was extracted with EtOAc. The organic phase was washed with saturated brine solution, dried over anhydrous Na₂SO₄ and concentrated in vacuo. The residue was purified by silica gel column chromatography (petroleum ether/EtOAc=1/5 to 1/2) to give methyl 1-[(1R)-2-(tert-butoxycarbonylamino)-1-methyl-ethyl]-5-(hydroxymethyl)pyrazole-3-carboxylate (800 mg, 84% yield). LCMS ESI (−) m/z 326.0 (M−H).

Step E: Preparation of methyl 1-[(1R)-2-(tert-butoxycarbonylamino)-1-methyl-ethyl]-5-(chloromethyl)pyrazole-3-carboxylate: To a solution of methyl 1-[(1R)-2-(tert-butoxycarbonylamino)-1-methyl-ethyl]-5-(hydroxymethyl)pyrazole-3-carboxylate (661 mg, 2.11 mmol) in DCM (4 mL) was added TEA (320 mg, 3.16 mmol), followed by MsCl (290 mg, 2.53 mmol) at 0° C. The mixture was stirred at 20° C. for 2 h. The mixture was poured into NaHCO₃ aqueous solution and the product was extracted with DCM. The combined organic layers were dried over anhydrous Na₂SO₄ and concentrated to dryness to give crude methyl 1-[(1R)-2-(tert-butoxycarbonylamino)-1-methyl-ethyl]-5-(chloromethyl)pyrazole-3-carboxylate (880 mg) which was used in the next step directly.

Step F: Preparation of 5-(tert-butyl)-2-methyl (R)-7-methyl-6,7-dihydropyrazolo[1,5-a]pyrazine-2,5(4H)-dicarboxylate: A solution of methyl 1-[(1R)-2-(tert-butoxycarbonylamino)-1-methyl-ethyl]-5-(chloromethyl)pyrazole-3-carboxylate (200 mg, 0.603 mmol) and sodium hydride (19 mg, 0.78 mmol) in DMF (4 mL) was stirred for 2 h at 25° C. The reaction was quenched with water and extracted with EtOAc. The combined organic layers were washed with brine, dried over Na₂SO₄, filtered and concentrated under reduced pressure. The crude was then purified by preparative TLC with (petroleum ether/EtOAc=3/1) to give 5-(tert-butyl) 2-methyl (R)-7-methyl-6,7-dihydropyrazolo[1,5-a]pyrazine-2,5(4H)-dicarboxylate (76 mg, 43% yield) as a white solid. LCMS ESI (+) m/z 296.2 (M+H).

Step G: Preparation of tert-butyl (R)-2-carbamoyl-7-methyl-6,7-dihydropyrazolo[1,5-a]pyrazine-5(4H)-carboxylate: A solution of 5-tert-butyl-2-methyl (7R)-7-methyl-6,7-dihydro-4H-pyrazolo[1,5-a]pyrazine-2,5-dicarboxylate (76 mg, 0.26 mmol) in 7 N ammonia in MeOH (5 mL) was stirred at 120° C. in a sealed tube for 20 h. After cooling, the solution was concentrated under vacuum to give the crude product (75 mg), which was used in the next step without further purification. LCMS ESI (+) m/z 281.2 (M+H).

Step H: Preparation of tert-butyl (R)-2-cyano-7-methyl-6,7-dihydropyrazolo[1,5-a]pyrazine-5(4H)-carboxylate: To a solution of tert-butyl (7R)-2-carbamoyl-7-methyl-6,7-dihydro-4H-pyrazolo[1,5-a]pyrazine-5-carboxylate (64 mg, 0.23 mmol) and triethylamine (0.32 mL, 2.3 mmol) in DCM (3 mL) in nitrogen atmosphere was added trifluoroacetic anhydride (0.097 mL, 0.69 mmol) at 0° C. The mixture was stirred at 15° C. for 1.5 h. The mixture was concentrated under reduced pressure. The mixture was diluted with water and the mixture was extracted with DCM. The combined organic layers were washed with brine, dried over Na₂SO₄, filtered and concentrated under reduced pressure. The crude was then purified by Preparative TLC (DCM/MeOH=100/1) to give tert-butyl (R)-2-cyano-7-methyl-6,7-dihydropyrazolo[1,5-a]pyrazine-5(4H)-carboxylate (33 mg, 55% yield) as a yellow solid. LCMS ESI (+) m/z 263.2 (M+H).

Step I: Preparation of tert-butyl (7R)-2-(7-(4-fluoro-2-isopropoxyphenyl)-4-hydroxythieno[3,2-c]pyridin-6-yl)-7-methyl-6,7-dihydropyrazolo[1,5-a]pyrazine-5(4H)-carboxylate: To a solution of N,N-diethyl-2-[(4-fluoro-2-isopropoxy-phenyl)methyl]thiophene-3-carboxamide (53 mg, 0.15 mmol) in THF (3 mL) was added n-butyllithium (0.080 mL, 2.5 M in hexanes, 0.20 mmol) dropwise at −70° C. under N₂ atmosphere. The mixture was stirred at −70° C. for 1 h. A solution of tert-butyl (7R)-2-cyano-7-methyl-6,7-dihydro-4H-pyrazolo[1,5-a]pyrazine-5-carboxylate (33 mg, 0.13 mmol) in THF (1 mL) was then added. The mixture was stirred at rt for additional 2 h. The reaction was diluted with water. 1 N aqueous HCl was added to adjust the pH to 5. The mixture was extracted with EtOAc, washed with brine, dried over Na₂SO₄, filtered and concentrated under reduced pressure. The residue was purified by preparative TLC (MeOH/DCM=1/20) to give tert-butyl (7R)-2-(7-(4-fluoro-2-isopropoxyphenyl)-4-hydroxythieno[3,2-c]pyridin-6-yl)-7-methyl-6,7-dihydropyrazolo[1,5-a]pyrazine-5(4H)-carboxylate (21 mg, 26% yield). LCMS ESI (+) m/z 539.2 (M+H).

Step J: Preparation of tert-butyl (7R)-2-(7-(4-fluoro-2-isopropoxyphenyl)-4-(((trifluoromethyl)sulfonyl)oxy)thieno[3,2-c]pyridin-6-yl)-7-methyl-6,7-dihydropyrazolo[1,5-a]pyrazine-5(4H)-carboxylate: To a solution of tert-butyl (7R)-2-[7-(4-fluoro-2-isopropoxy-phenyl)-4-hydroxy-thieno[3,2-c]pyridin-6-yl]-7-methyl-6,7-dihydro-4H-pyrazolo[1,5-a]pyrazine-5-carboxylate (21 mg, 0.039 mmol) and pyridine (0.025 mL, 0.31 mmol) in DCM (3 mL) was added trifluoromethanesulfonic anhydride (0.0099 mL, 0.059 mmol) at 0° C. The mixture was stirred at rt for 1 h. The mixture was diluted with water and extracted with DCM. The combined organic layers were washed with brine, dried over Na₂SO₄, filtered and concentrated under reduced pressure. The residue was purified by preparative TLC (petroleum ether/EtOAc=8/1) to give tert-butyl (7R)-2-(7-(4-fluoro-2-isopropoxyphenyl)-4-(((trifluoromethyl)sulfonyl) oxy)thieno[3,2-c]pyridin-6-yl)-7-methyl-6,7-dihydropyrazolo[1,5-a]pyrazine-5(4H)-carboxylate (25 mg, 96% yield). LCMS ESI (+) m/z 671.2 (M+H).

Step K: Preparation of 7-(4-fluoro-2-isopropoxyphenyl)-6-((R)-7-methyl-4,5,6,7-tetrahydropyrazolo[1,5-a]pyrazin-2-yl)thieno[3,2-c]pyridin-4-yl trifluoromethanesulfonate: To a solution of tert-butyl (7R)-2-[7-(4-fluoro-2-isopropoxy-phenyl)-4-(trifluoromethylsulfonyloxy)thieno[3,2-c]pyridin-6-yl]-7-methyl-6,7-dihydro-4H-pyrazolo[1,5-a]pyrazine-5-carboxylate (25 mg, 0.037 mmol) in DCM (3 mL) was added TFA (1 mL). The mixture was stirred at rt for 1 h. The reaction was concentrated to dryness to give 7-(4-fluoro-2-isopropoxyphenyl)-6-((R)-7-methyl-4,5,6,7-tetrahydropyrazolo[1,5-a]pyrazin-2-yl)thieno[3,2-c]pyridin-4-yl trifluoromethanesulfonate (29 mg) as a trifluoroacetic acid salt, which was used in the next step without further purification. LCMS ESI (+) m/z 571.1 (M+H).

Step L: Preparation of 6-((R)-5-acryloyl-7-methyl-4,5,6,7-tetrahydropyrazolo[1,5-a]pyrazin-2-yl)-7-(4-fluoro-2-isopropoxyphenyl)thieno[3,2-c]pyridin-4-yl trifluoromethanesulfonate: To a solution of [7-(4-fluoro-2-isopropoxy-phenyl)-6-[(7R)-7-methyl-4,5,6,7-tetrahydropyrazolo[1,5-a]pyrazin-2-yl]thieno[3,2-c]pyridin-4-yl]trifluoromethanesulfonate (29 mg, 0.037 mmol) and triethylamine (38 mg, 0.37 mmol) in dry DCM (3 mL) was added a solution of acrylic anhydride (5.0 mg, 0.056 mmol) in dry DCM (1 mL) at −60° C. The mixture was stirred at this temperature for 1 h. The mixture was concentrated under reduced pressure. The residue was purified by preparative TLC (MeOH/DCM=1/20) to give 6-((R)-5-acryloyl-7-methyl-4,5,6,7-tetrahydropyrazolo[1,5-a]pyrazin-2-yl)-7-(4-fluoro-2-isopropoxyphenyl)thieno[3,2-c]pyridin-4-yl trifluoromethanesulfonate (21 mg, 90% yield over two steps). LCMS ESI (+) m/z 625.1 (M+H).

Step M: Preparation of 1-((7R)-2-(7-(4-fluoro-2-isopropoxyphenyl)-4-(1-methyl-1H-pyrazol-4-yl)thieno[3,2-c]pyridin-6-yl)-7-methyl-6,7-dihydropyrazolo[1,5-a]pyrazin-5(4H)-yl)prop-2-en-1-one: A solution of [7-(4-fluoro-2-isopropoxy-phenyl)-6-[(7R)-7-methyl-5-prop-2-enoyl-6,7-dihydro-4H-pyrazolo[1,5-a]pyrazin-2-yl]thieno[3,2-c]pyridin-4-yl]trifluoromethanesulfonate (21 mg, 0.034 mmol), 1-methyl-4-(4,4,5,5-tetramethyl-1,3,2-dioxaborolan-2-yl)pyrazole (9.1 mg, 0.044 mmol), tetrakis(triphenylphosphine)palladium(0) (3.9 mg, 0.0034 mmol) and sodium carbonate (7.1 mg, 0.067 mmol) in 1,4-dioxane (1.2 mL) and water (0.3 mL) was stirred for 4 h at 100° C. under N₂ atmosphere. The mixture was diluted with water and the mixture was extracted with EtOAc. The combined organics were washed with saturated brine solution, dried over Na₂SO₄ and concentrated to dryness. The crude was then purified by preparative HPLC to give 1-((7R)-2-(7-(4-fluoro-2-isopropoxyphenyl)-4-(1-methyl-1H-pyrazol-4-yl)thieno[3,2-c]pyridin-6-yl)-7-methyl-6,7-dihydropyrazolo[1,5-a]pyrazin-5(4H)-yl)prop-2-en-1-one (12 mg, 62% yield) as a trifluoroacetic acid salt. ¹H NMR (400 MHz, CD₃OD) δ 8.66 (d, J=2.0 Hz, 1H), 8.34 (d, J=2.1 Hz, 1H), 8.07-8.15 (m, 2H), 7.38-7.44 (m, 1H), 6.76-7.03 (m, 3H), 6.31 (d, J=15.1 Hz, 1H), 5.74-5.92 (m, 2H), 4.72-4.80 (m, 2H), 4.53-4.59 (m, 2H), 4.19-4.90 (m, 5H), 1.55 (d, J=4.2 Hz, 3H), 1.08 (t, J=6.8 Hz, 3H), 0.91 (dd, J=5.7, 19.4 Hz, 3H). LCMS ESI (+) m/z 557.2 (M+H).

### Synthetic Example 53: Synthesis of 1-[3-[3-(4-fluoro-2-isopropoxy-phenyl)-6-(1-methylpyrazol-4-yl)-5-(trifluoromethyl)-2-pyridyl]-7,8-dihydro-5H-1,6-naphthyridin-6-yl]prop-2-en-1-one (Compound 360)

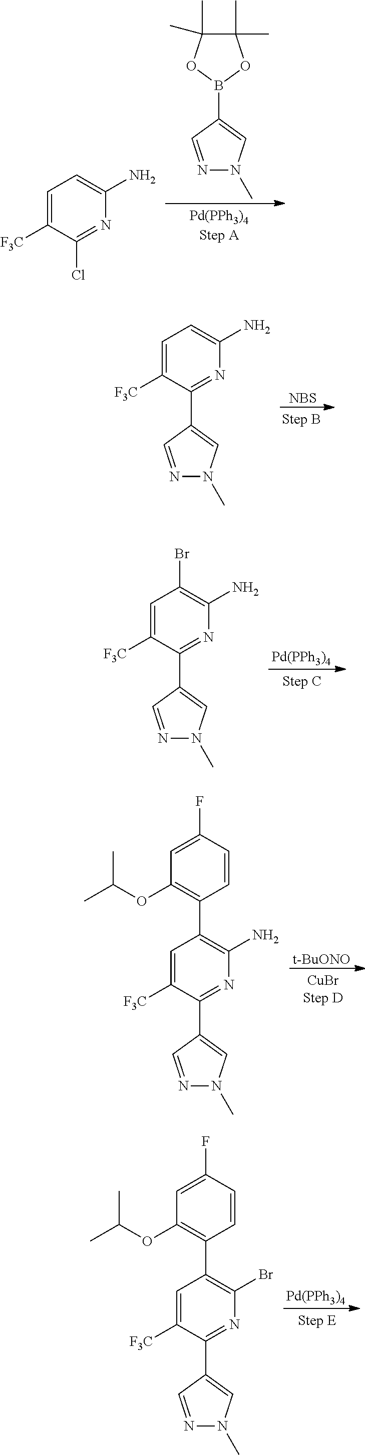

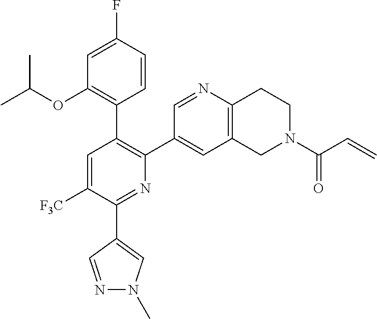 [SMILES: C=CC(=O)N1CCc2ncc(-c3nc(-c4cnn(C)c4)c(C(F)(F)F)cc3-c3ccc(F)cc3OC(C)C)cc2C1]

Step A: Preparation of 6-(1-methylpyrazol-4-yl)-5-(trifluoromethyl)pyridin-2-amine: To a solution of 6-chloro-5-(trifluoromethyl)pyridin-2-amine (0.500 g, 2.54 mmol) in 1,4-dioxane (10 mL)/water (2.5 mL) were added 1-methyl-4-(4,4,5,5-tetramethyl-1,3,2-dioxaborolan-2-yl)pyrazole (1.06 g, 5.09 mmol), Pd(PPh₃)₄(0.294 g, 0.254 mmol) and Na₂CO₃ (0.809 g, 7.63 mmol). The mixture was stirred at 100° C. for 16 h under Ar. The reaction was quenched with water and extracted with DCM twice. The combined organic layers were washed with brine, dried and concentrated. The crude was purified by preparative TLC (petroleum ether/EtOAc=1/2) to afford 6-(1-methylpyrazol-4-yl)-5-(trifluoromethyl)pyridin-2-amine (0.780 g, 76% yield). ¹H NMR (400 MHz, CD₃OD) δ 7.92 (s, 1H), 7.78 (s, 1H), 7.68 (d, J=8.8 Hz, 1H), 6.47 (d, J=8.8 Hz, 1H), 3.93 (s, 3H), 3.35 (s, 2H).

Step B: Preparation of 3-bromo-6-(1-methylpyrazol-4-yl)-5-(trifluoromethyl)pyridin-2-amine: To a solution of 6-(1-methylpyrazol-4-yl)-5-(trifluoromethyl)pyridin-2-amine (780 mg, 3.22 mmol) in DMF (5 mL) was added N-bromosuccinimide (573 mg, 3.22 mmol). The mixture was stirred at 10° C. for 2 h. The reaction was quenched with saturated Na₂S₂O₃ solution and extracted with EtOAc twice. The combined organic layers were washed with saturate brine, dried and concentrated. The crude was purified by column chromatography (petroleum ether/EtOAc=1/1) to afford 3-bromo-6-(1-methylpyrazol-4-yl)-5-(trifluoromethyl)pyridin-2-amine (1.00 g, 97% yield). ¹H NMR (400 MHz, CD₃OD) δ 7.97 (s, 1H), 7.94 (s, 1H), 7.84 (s, 1H), 3.93 (s, 3H).

Step C: Preparation of 3-(4-fluoro-2-isopropoxy-phenyl)-6-(1-methylpyrazol-4-yl)-5-(trifluoromethyl)pyridin-2-amine: To a solution of 3-bromo-6-(1-methylpyrazol-4-yl)-5-(trifluoromethyl)pyridin-2-amine (600 mg, 1.87 mmol) in 1,4-dioxane (10 mL)/water (1.5 mL) were added (4-fluoro-2-isopropoxy-phenyl)boronic acid (555 mg, 2.80 mmol), Pd(PPh₃)₄(216 mg, 0.187 mmol) and Na₂CO₃ (594 mg, 5.61 mmol). The reaction was stirred at 100° C. for 3 h under Ar. The mixture was concentrated and purified by column chromatography (petroleum ether/EtOAc=1/1 to 1/2) to afford 3-(4-fluoro-2-isopropoxy-phenyl)-6-(1-methylpyrazol-4-yl)-5-(trifluoromethyl)pyridin-2-amine (600 mg, 81% yield). LCMS ESI (+) m/z 395.3 (M+H).

Step D: Preparation of 2-bromo-3-(4-fluoro-2-isopropoxy-phenyl)-6-(1-methylpyrazol-4-yl)-5-(trifluoromethyl)pyridine: To a solution of 3-(4-fluoro-2-isopropoxy-phenyl)-6-(1-methylpyrazol-4-yl)-5-(trifluoromethyl)pyridin-2-amine (340 mg, 0.862 mmol) in acetonitrile (6 mL) was added tert-butyl nitrite (267 mg, 2.59 mmol) dropwise at −10° C. The mixture was stirred at −10° C. for 0.5 h. CuBr (370 mg, 2.59 mmol) was added slowly at −10° C. The resulting mixture was stirred at 55° C. for 2 h. The reaction was quenched with saturated NH₄Cl solution and extracted with EtOAc. The combined organic layers were washed with brine, dried and concentrated. The crude was purified by preparative TLC (petroleum ether/EtOAc=3/1) to afford 2-bromo-3-(4-fluoro-2-isopropoxy-phenyl)-6-(1-methylpyrazol-4-yl)-5-(trifluoromethyl)pyridine (30 mg, 8% yield). ¹H-NMR (400 MHz, CDCl₃): 8.04 (s, 1H), 7.98 (s, 1H), 7.82 (s, 1H), 7.19 (t, J=7.6 Hz, 1H), 6.69-6.76 (m, 2H), 4.45-4.55 (m, 1H), 3.98 (s, 3H), 1.28 (d, J=6.0 Hz, 6H).

Step E: Preparation of 1-[3-[3-(4-fluoro-2-isopropoxy-phenyl)-6-(1-methylpyrazol-4-yl)-5-(trifluoromethyl)-2-pyridyl]-7,8-dihydro-5H-1,6-naphthyridin-6-yl]prop-2-en-1-one: To a solution of 2-bromo-3-(4-fluoro-2-isopropoxy-phenyl)-6-(1-methylpyrazol-4-yl)-5-(trifluoromethyl)pyridine (8.0 mg, 0.018 mmol) in 1,4-dioxane (1 mL)/water (0.2 mL) were added (6-prop-2-enoyl-7,8-dihydro-5H-1,6-naphthyridin-3-yl)boronic acid (8.1 mg, 0.035 mmol), Pd(PPh₃)₄(2.0 mg, 0.0018 mmol) and Na₂CO₃ (5.6 mg, 0.052 mmol). The reaction was stirred at 100° C. for 1 h under Ar. After cooling, the mixture was filtered and purified by preparative HPLC to afford 1-[3-[3-(4-fluoro-2-isopropoxy-phenyl)-6-(1-methylpyrazol-4-yl)-5-(trifluoromethyl)-2-pyridyl]-7,8-dihydro-5H-1,6-naphthyridin-6-yl]prop-2-en-1-one (5.1 mg, 51% yield) as a trifluoroacetic acid salt. ¹H NMR (400 MHz, CD₃OD) δ 8.45 (s, 1H), 8.22 (s, 1H), 8.02-8.10 (m, 3H), 7.34 (t, J=7.2 Hz, 1H), 6.75-6.95 (m, 3H), 6.25-6.29 (m, 1H), 5.81 (d, J=10.4 Hz, 1H), 4.70-4.75 (m, 2H), 4.43 (t, J=6.4 Hz, 1H), 4.00 (s, 3H), 3.95 (s, 2H), 3.10 (t, J=6.0 Hz, 2H), 0.97 (s, 6H). LCMS ESI (+) m/z 566.3 (M+H).

### Synthetic Example 54: Synthesis of 5-[7-(2,4-difluoro-6-isopropoxy-phenyl)-6-(7-prop-2-enoyl-6,8-dihydro-5H-1,7-naphthyridin-2-yl)thieno[3,2-c]pyridin-4-yl]isoindolin-1-one (Compound 411)

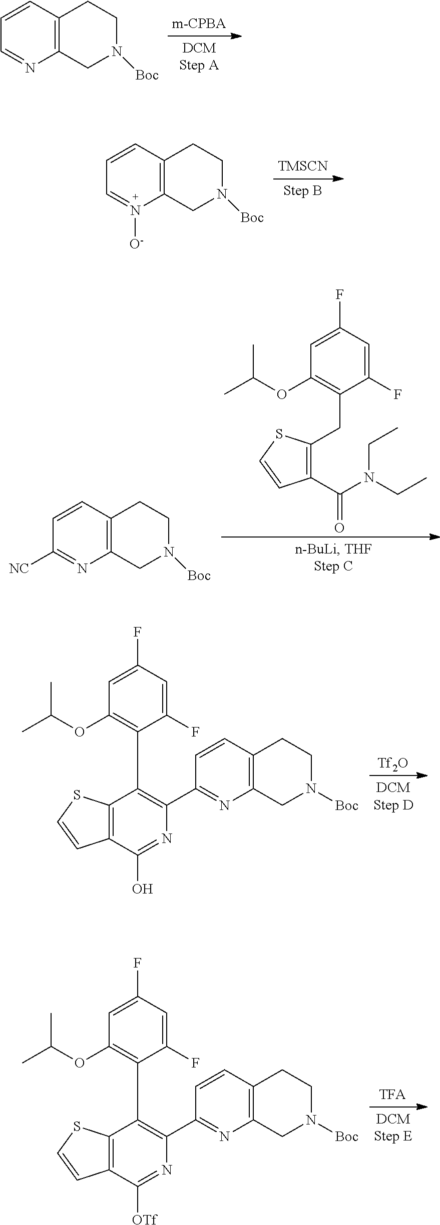

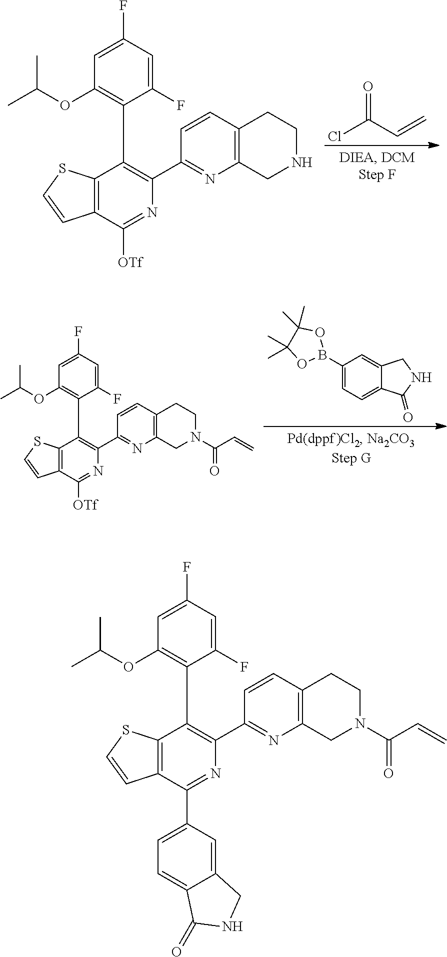

Step A: Preparation of tert-butyl 1-oxido-6,8-dihydro-5H-1,7-naphthyridin-1-ium-7-carboxylate: To a stirred solution of tert-butyl 6,8-dihydro-5H-1,7-naphthyridine-7-carboxylate (760 mg, 3.24 mmol) in DCM (5 mL) was added m-CPBA (1120 mg, 6.49 mmol). The mixture was stirred at rt for 16 h. The aqueous layer was extracted with DCM. The combined organics were then separated, dried (MgSO₄) and concentrated to dryness. The residue was purified by flash column chromatography (5% MeOH in DCM) to give tert-butyl 1-oxido-6,8-dihydro-5H-1,7-naphthyridin-1-ium-7-carboxylate (880 mg, 87% yield). LCMS ESI (+) m/z 250.9 (M+H).

Step B: Preparation of tert-butyl 2-cyano-6,8-dihydro-5H-1,7-naphthyridine-7-carboxylate: To a stirred solution of tert-butyl 1-oxido-6,8-dihydro-5H-1,7-naphthyridin-1-ium-7-carboxylate (800 mg, 3.20 mmol) in DCM (5 mL) were added trimethylsilyl cyanide (0.860 mL, 6.39 mmol) and dimethylcarbamoyl chloride (0.590 mL, 6.39 mmol). The solution was stirred at 40° C. for 16 h. The aqueous layer was extracted with DCM. The organics were then separated, dried over MgSO₄, concentrated to dryness and purified by flash column chromatography eluting with 40% EtOAc in hexanes to give tert-butyl 2-cyano-6,8-dihydro-5H-1,7-naphthyridine-7-carboxylate (510 mg, 49% yield). LCMS ESI (+) m/z 260.0 (M+H).

Step C: Preparation of tert-butyl 2-[7-(2,4-difluoro-6-isopropoxy-phenyl)-4-hydroxy-thieno[3,2-c]pyridin-6-yl]-6,8-dihydro-5H-1,7-naphthyridine-7-carboxylate: To a solution of tert-butyl 2-cyano-6,8-dihydro-5H-1,7-naphthyridine-7-carboxylate (500 mg, 1.93 mmol) in THF (14 mL) was added 2-[(2,4-difluoro-6-isopropoxy-phenyl)methyl]-N,N-diethyl-thiophene-3-carboxamide (872 mg, 2.37 mmol) at −60° C. under Ar atmosphere. The mixture was stirred at rt for 3 h. The reaction was quenched with water. After adjusting the pH to 5, the mixture was extracted with EtOAc. The combined organics were dried over MgSO₄, concentrated to dryness and purified by flash column chromatography eluting with 50% EtOAc in hexanes to give tert-butyl 2-[7-(2,4-difluoro-6-isopropoxy-phenyl)-4-hydroxy-thieno[3,2-c]pyridin-6-yl]-6,8-dihydro-5H-1,7-naphthyridine-7-carboxylate (600 mg, 51% yield). LCMS ESI (+) m/z 553.8 (M+H).

Step D: Preparation of tert-butyl 2-[7-(2,4-difluoro-6-isopropoxy-phenyl)-4-(trifluoromethylsulfonyloxy)thieno[3,2-c]pyridin-6-yl]-6,8-dihydro-5H-1,7-naphthyridine-7-carboxylate: To a solution of tert-butyl 2-[7-(2,4-difluoro-6-isopropoxy-phenyl)-4-hydroxy-thieno[3,2-c]pyridin-6-yl]-6,8-dihydro-5H-1,7-naphthyridine-7-carboxylate (600 mg, 1.08 mmol) in DCM (15 mL) were added Tf₂O (1070 mg, 3.79 mmol) and pyridine (1.20 mL, 10.8 mmol). The mixture was stirred at 25° C. for 1 h. The reaction was concentrated to dryness and purified by flash column chromatography eluting with 20% EtOAc in hexanes to give tert-butyl 2-[7-(2,4-difluoro-6-isopropoxy-phenyl)-4-(trifluoromethylsulfonyloxy)thieno[3,2-c]pyridin-6-yl]-6,8-dihydro-5H-1,7-naphthyridine-7-carboxylate (580 mg, 70% yield). LCMS ESI (+) m/z 685.7 (M+H).

Step E: Preparation of [7-(2,4-difluoro-6-isopropoxy-phenyl)-6-(5,6,7,8-tetrahydro-1,7-naphthyridin-2-yl)thieno[3,2-c]pyridin-4-yl]trifluoromethanesulfonate: A solution of [6-[6-[(tert-butoxycarbonylamino)methyl]-5-methyl-2-pyridyl]-7-(2,4-difluoro-6-isopropoxy-phenyl)thieno[3,2-c]pyridin-4-yl]trifluoromethanesulfonate (580 mg, 0.861 mmol) in TFA (6.6 mL) and DCM (20 mL) was stirred at 25° C. for 2 h. The mixture was concentrated to give the crude product [7-(2,4-difluoro-6-isopropoxy-phenyl)-6-(5,6,7,8-tetrahydro-1,7-naphthyridin-2-yl)thieno[3,2-c]pyridin-4-yl]trifluoromethanesulfonate (580 mg, crude), which was used in the next step without further purification. LCMS ESI (+) m/z 586.0 (M+H).

Step F: Preparation of [7-(2,4-difluoro-6-isopropoxy-phenyl)-6-(7-prop-2-enoyl-6,8-dihydro-5H-1,7-naphthyridin-2-yl)thieno[3,2-c]pyridin-4-yl]trifluoromethanesulfonate: To a stirred solution of [7-(2,4-difluoro-6-isopropoxy-phenyl)-6-(5,6,7,8-tetrahydro-1,7-naphthyridin-2-yl)thieno[3,2-c]pyridin-4-yl]trifluoromethanesulfonate (580 mg, 0.990 mmol) in DCM (15 mL) was added acryloyl chloride (0.12 mL, 1.5 mmol) and DIEA (0.71 mL, 4.0 mmol). The solution was stirred at −60° C. for 1 h. The reaction was quenched with NaHCO₃, and the residue was taken up in DCM. The combined organics were washed with water and brine, dried and concentrated. The residue was purified by flash column chromatography eluting with 50% EtOAc in hexanes to give [7-(2,4-difluoro-6-isopropoxy-phenyl)-6-(7-prop-2-enoyl-6,8-dihydro-5H-1,7-naphthyridin-2-yl)thieno[3,2-c]pyridin-4-yl]trifluoromethanesulfonate (570 mg, 81% yield). LCMS ESI (+) m/z 640.1 (M+H).

Step G: Preparation of 5-[7-(2,4-difluoro-6-isopropoxy-phenyl)-6-(7-prop-2-enoyl-6,8-dihydro-5H-1,7-naphthyridin-2-yl)thieno[3,2-c]pyridin-4-yl]isoindolin-1-one: A mixture of [7-(2,4-difluoro-6-isopropoxy-phenyl)-6-(7-prop-2-enoyl-6,8-dihydro-5H-1,7-naphthyridin-2-yl)thieno[3,2-c]pyridin-4-yl]trifluoromethanesulfonate (70 mg, 0.11 mmol), 5-(4,4,5,5-tetramethyl-1,3,2-dioxaborolan-2-yl)isoindolin-1-one (43 mg, 0.16 mmol), Na₂CO₃ (23 mg, 0.22 mmol) and Pd(dppf)Cl₂ (12 mg, 0.016 mmol) in 1,4-dioxane (5 mL) and water (0.5 mL) was stirred under argon at 100° C. for 1 h. The reaction was concentrated to dryness. The residue was then purified by flash column chromatography eluting with 5% MeOH in DCM followed by reverse phase preparative HPLC to afford 5-[7-(2,4-difluoro-6-isopropoxy-phenyl)-6-(7-prop-2-enoyl-6,8-dihydro-5H-1,7-naphthyridin-2-yl)thieno[3,2-c]pyridin-4-yl]isoindolin-1-one (17 mg, 24% yield) as a white solid, ¹H NMR (400 MHz, CD₃OD) δ 8.24 (s, 1H), 8.18 (d, J=7.8 Hz, 1H), 8.03 (d, J=7.8 Hz, 1H), 7.87-7.79 (m, 2H), 7.76 (d, J=5.4 Hz, 1H), 7.66-7.60 (m, 1H), 6.88 (dd, J=16.6, 10.8 Hz, 1H), 6.74 (d, J=11.0 Hz, 1H), 6.65 (dd, J=17.8, 7.4 Hz, 1H), 6.26 (dd, J=16.8, 8.8 Hz, 1H), 5.82 (t, J=11.4 Hz, 1H), 4.65 (s, 2H), 4.62-4.44 (m, 3H), 3.95-3.85 (m, 2H), 2.93 (dd, J=11.0, 5.8 Hz, 2H), 1.10 (t, J=6.4 Hz, 3H), 0.90 (dd, J=20.4, 5.8 Hz, 3H). LCMS ESI (+) m/z 623.2 (M+H).

### Synthetic Example 55: Synthesis of 1-[2-[7-[2,4-difluoro-6-(2-methoxyethoxy)phenyl]-3-fluoro-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (Compound 434)

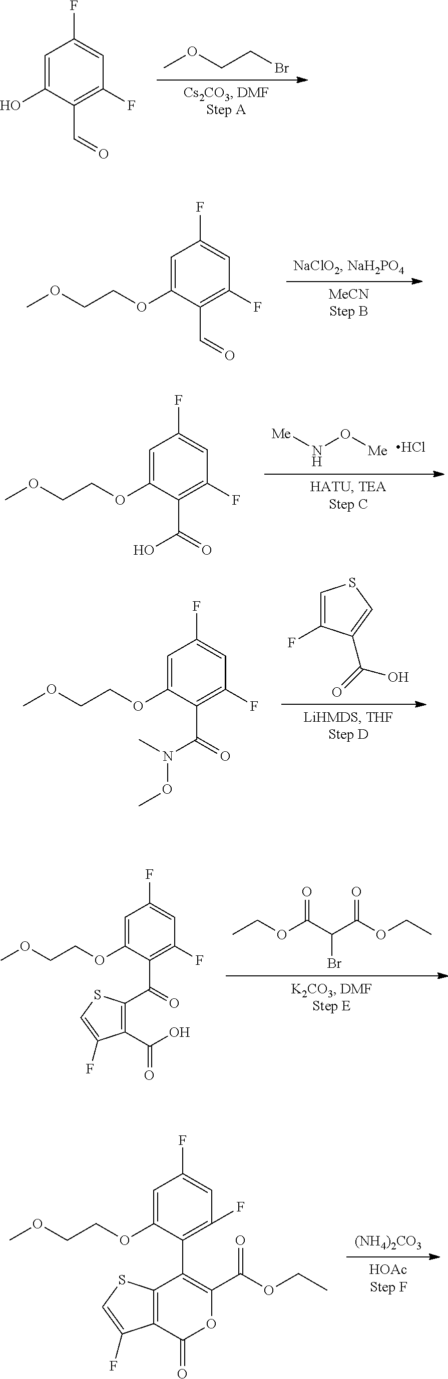

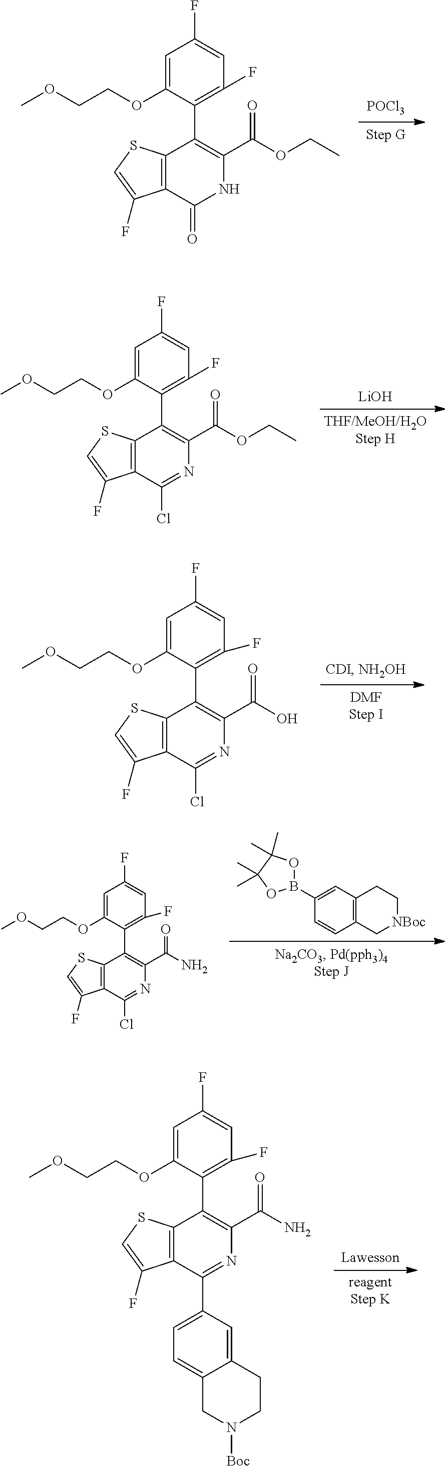

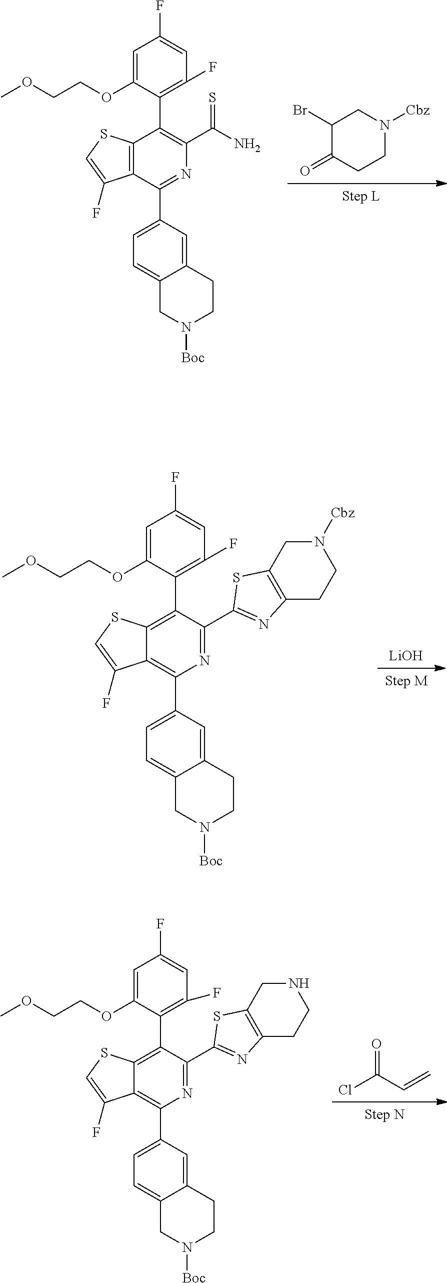

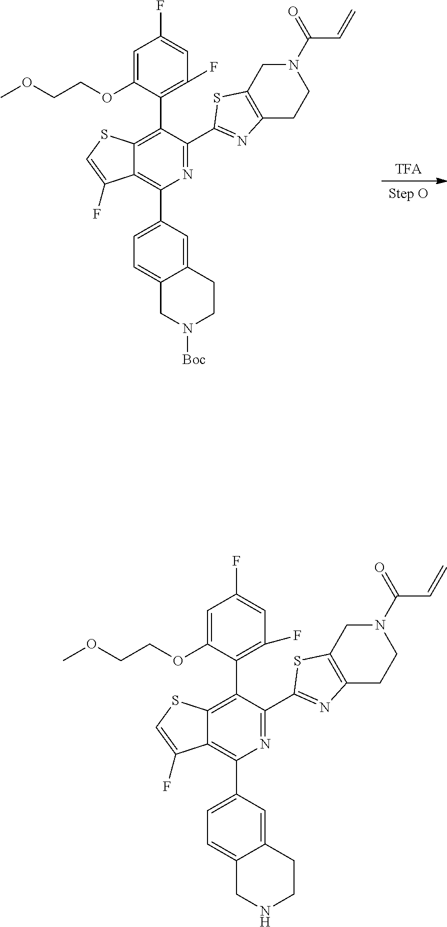

Step A: Preparation of 2,4-difluoro-6-(2-methoxyethoxy)benzaldehyde: To a solution of 2,4-difluoro-6-hydroxy-benzaldehyde (3.00 g, 19.0 mmol) in DMF (30 mL) was added 1-bromo-2-methoxy-ethane (3.42 g, 24.7 mmol) and cesium carbonate (12.4 g, 38.0 mmol). The reaction mixture was stirred at 70° C. for 3 hours. The reaction mixture was concentrated, diluted with EtOAc and filtered. The filtrate was washed with saturated brine solution twice, dried over anhydrous Na₂SO₄, filtered and concentrated to dryness to give 2,4-difluoro-6-(2-methoxyethoxy)benzaldehyde (4.0 g, 98% yield) as a light-yellow solid. The crude was used for the next step without further purification. LCMS ESI (+) m/z 217.1 (M+H).

Step B: Preparation of 2,4-difluoro-6-(2-methoxyethoxy)benzoic acid: To a solution of 2,4-difluoro-6-(2-methoxyethoxy)benzaldehyde (1.60 g, 7.40 mmol) in MeCN/water (24 mL/8 mL) were added NaH₂PO₄ 2H₂O (11.6 g, 74.0 mmol) and NaClO₂ (2.67 g, 29.6 mmol). The reaction mixture was stirred at 30° C. for 2 hours. The reaction mixture was diluted with water (30 mL) and extracted with EtOAc twice. The combined organic layers were washed with saturated brine solution, dried over anhydrous Na₂SO₄, filtered and concentrated to dryness to give 2,4-difluoro-6-(2-methoxyethoxy)benzoic acid (1.20 g, 70% yield) as a light-yellow solid. ¹H NMR (400 MHz, DMSO-d6) 13.39 (s, 1H), 6.87-6.96 (m, 2H), 4.18 (t, J=4.4 Hz, 2H), 3.68 (t, J=4.4 Hz, 2H), 3.30 (s, 3H). LCMS ESI (+) m/z 233.0 (M+H).

Step C: Preparation of 2,4-difluoro-N-methoxy-6-(2-methoxyethoxy)-N-methyl-benzamide: To a solution of 2,4-difluoro-6-(2-methoxyethoxy)benzoic acid (1.20 g, 5.16 mmol) in DCM (10 mL) was added HATU (2.55 g, 6.71 mmol) and TEA (1.56 g, 15.5 mmol). The mixture was stirred at rt for 30 min. Then N,O-dimethylhydroxylamine hydrochloride (0.750 g, 7.75 mmol) was added. The resulting mixture was stirred at rt for 30 min. The mixture was diluted with NaHCO₃ saturated aqueous solution (20 mL) and the product was extracted with EtOAc twice. The combined organic layers were washed with brine solution, dried over anhydrous Na₂SO₄, filtered and concentrated. The residue was purified by silica gel column chromatography (ethyl acetate:hexane=2:1) to give 2,4-difluoro-N-methoxy-6-(2-methoxyethoxy)-N-methyl-benzamide (1.3 g, 92% yield). LCMS ESI (+) m/z 276.2 (M+H).

Step D: Preparation of 2-[2,4-difluoro-6-(2-methoxyethoxy)benzoyl]-4-fluoro-thiophene-3-carboxylic acid: To a solution of 4-fluorothiophene-3-carboxylic acid (0.690 g, 4.72 mmol) in THF (6 mL) at −60° C. under argon atmosphere was added LiHMDS (10.5 mL, 1.0 M solution in THF, 10.5 mmol). The mixture was stirred at −60° C. for 30 min. Then 2,4-difluoro-N-methoxy-6-(2-methoxyethoxy)-N-methyl-benzamide (1.30 g, 4.72 mmol) was added. The mixture was warmed to 30° C. and stirred for 4 h under argon atmosphere. 1 N aqueous HCl solution was added to the reaction mixture to adjust the pH 5. The mixture was diluted with water and extracted with EtOAc twice. The combined organic layers were washed with brine solution, dried over anhydrous Na₂SO₄, filtered and concentrated. The residue was purified by reserve-phase column chromatography to give 2-[2,4-difluoro-6-(2-methoxyethoxy)benzoyl]-4-fluoro-thiophene-3-carboxylic acid (180 mg, 11% yield). LCMS ESI (+) m/z 360.9 (M+H).

Step E: Preparation of ethyl 7-[2,4-difluoro-6-(2-methoxyethoxy)phenyl]-3-fluoro-4-oxo-thieno[3,2-c]pyran-6-carboxylate: To a solution of 2-[2,4-difluoro-6-(2-methoxyethoxy)benzoyl]-4-fluoro-thiophene-3-carboxylic acid (180 mg, 0.490 mmol) in DMF (5 mL) was added diethyl 2-bromopropanedioate (155 mg, 0.640 mmol) and K₂CO₃ (135 mg, 0.980 mmol). The mixture was stirred at 50° C. for 64 h. The reaction mixture was diluted with EtOAc (50 mL) and H₂O (50 mL). The organic phase was separated and washed with saturated brine solution, dried over anhydrous Na₂SO₄, filtered and concentrated. The residue was purified by preparative TLC (ethyl acetate:hexane=1:1) to give ethyl 7-[2,4-difluoro-6-(2-methoxyethoxy)phenyl]-3-fluoro-4-oxo-thieno[3,2-c]pyran-6-carboxylate (200 mg, 95% yield). LCMS ESI (+) m/z 428.9 (M+H).

Step F: Preparation of ethyl 7-[2,4-difluoro-6-(2-methoxyethoxy)phenyl]-3-fluoro-4-oxo-5H-thieno[3,2-c]pyridine-6-carboxylate: To a solution of ethyl 7-[2,4-difluoro-6-(2-methoxyethoxy)phenyl]-3-fluoro-4-oxo-thieno[3,2-c]pyran-6-carboxylate (200 mg, 0.460 mmol) in acetic acid (2 mL) was added (NH₄)₂CO₃ (898 mg, 9.34 mmol). The mixture was stirred at 90° C. for 12 h. The reaction mixture was concentrated, diluted with brine and extracted with EtOAc twice. The combined organic layers were washed with brine solution, dried over anhydrous Na₂SO₄, filtered and concentrated. The residue was purified by preparative TLC (ethyl acetate:hexane=1:1) to give ethyl 7-[2,4-difluoro-6-(2-methoxyethoxy)phenyl]-3-fluoro-4-oxo-5H-thieno[3,2-c]pyridine-6-carboxylate (130 mg, 66% yield). LCMS ESI (+) m/z 427.9 (M+H).

Step G: Preparation of ethyl 4-chloro-7-[2,4-difluoro-6-(2-methoxyethoxy)phenyl]-3-fluoro-thieno[3,2-c]pyridine-6-carboxylate: A solution of ethyl 7-[2,4-difluoro-6-(2-methoxyethoxy)phenyl]-3-fluoro-4-oxo-5H-thieno[3,2-c]pyridine-6-carboxylate (130 mg, 0.300 mmol) in POCl₃ (3 mL) was stirred at 90° C. for 2 h. The reaction mixture was cooled to rt and concentrated. The residue was dissolved in EtOAc, washed with brine, dried over Na₂SO₄, filtered and concentrated. The resulting residue was purified by preparative TLC (ethyl acetate:hexane=1:1) to give ethyl 4-chloro-7-[2,4-difluoro-6-(2-methoxyethoxy)phenyl]-3-fluoro-thieno[3,2-c]pyridine-6-carboxylate (130 mg, 97% yield). LCMS ESI (+) m/z 445.9 (M+H).

Step H: Preparation of 4-chloro-7-[2,4-difluoro-6-(2-methoxyethoxy)phenyl]-3-fluoro-thieno[3,2-c]pyridine-6-carboxylic acid: To a solution of ethyl 4-chloro-7-[2,4-difluoro-6-(2-methoxyethoxy)phenyl]-3-fluoro-thieno[3,2-c]pyridine-6-carboxylate (130 mg, 0.290 mmol) in THF/MeOH/water (3 mL/1 mL/1 mL) was added lithium hydroxide (69.8 mg, 2.91 mmol). The mixture was stirred at rt for 2 h. The mixture was diluted with water and 1 N HCl aqueous solution was added to adjust the pH to 3. The solution was extracted with EtOAc twice. The combined organic layers were washed with brine solution, dried over anhydrous Na₂SO₄, filtered and concentrated to dryness to give 4-chloro-7-[2,4-difluoro-6-(2-methoxyethoxy)phenyl]-3-fluoro-thieno[3,2-c]pyridine-6-carboxylic acid (120 mg, 99% yield), which was used for the next step without further purification. LCMS ESI (+) m/z 417.9 (M+H).

Step I: Preparation of 4-chloro-7-[2,4-difluoro-6-(2-methoxyethoxy)phenyl]-3-fluoro-thieno[3,2-c]pyridine-6-carboxamide: To a solution of 4-chloro-7-[2,4-difluoro-6-(2-methoxyethoxy)phenyl]-3-fluoro-thieno[3,2-c]pyridine-6-carboxylic acid (120 mg, 0.280 mmol) in DMF (3 mL) was added N,N′-carbonyldiimidazole (466 mg, 2.87 mmol). The mixture was stirred at rt for 0.5 h. The mixture was added to aqueous ammonia solution (5 mL), and the resulting mixture was stirred at rt for additional 1 h. The solution was diluted with water and extracted with EtOAc twice. The combined organic layers were washed with brine solution, dried over anhydrous Na₂SO₄, filtered and concentrated. The residue was purified by preparative TLC (ethyl acetate:hexane=2:1) to give 4-chloro-7-[2,4-difluoro-6-(2-methoxyethoxy)phenyl]-3-fluoro-thieno[3,2-c]pyridine-6-carboxamide (80 mg, 67% yield). LCMS ESI (+) m/z 416.9 (M+H).

Step J: Preparation of tert-butyl 6-[6-carbamoyl-7-[2,4-difluoro-6-(2-methoxyethoxy)phenyl]-3-fluoro-thieno[3,2-c]pyridin-4-yl]-3,4-dihydro-1H-isoquinoline-2-carboxylate: To a solution of 4-chloro-7-[2,4-difluoro-6-(2-methoxyethoxy)phenyl]-3-fluoro-thieno[3,2-c]pyridine-6-carboxamide (80 mg, 0.19 mmol) and tert-butyl 6-(4,4,5,5-tetramethyl-1,3,2-dioxaborolan-2-yl)-3,4-dihydro-1H-isoquinoline-2-carboxylate (103 mg, 0.280 mmol) in 1,4-dioxane (4 mL)/water (0.4 mL) was added Na₂CO₃ (61 mg, 0.57 mmol) and tetrakis(triphenylphosphine)palladium (22 mg, 0.019 mmol). The reaction mixture was stirred at 100° C. for 2 h under Ar. The reaction mixture was concentrated and purified by preparative TLC (ethyl acetate: hexane=2:1) to give tert-butyl 6-[6-carbamoyl-7-[2,4-difluoro-6-(2-methoxyethoxy)phenyl]-3-fluoro-thieno[3,2-c]pyridin-4-yl]-3,4-dihydro-1H-isoquinoline-2-carboxylate (55 mg, 47% yield). LCMS ESI (+) m/z 614.0 (M+H).

Step K: Preparation of tert-butyl 6-[6-carbamothioyl-7-[2,4-difluoro-6-(2-methoxyethoxy)phenyl]-3-fluoro-thieno[3,2-c]pyridin-4-yl]-3,4-dihydro-1H-isoquinoline-2-carboxylate: To a solution of tert-butyl 6-[6-carbamoyl-7-[2,4-difluoro-6-(2-methoxyethoxy)phenyl]-3-fluoro-thieno[3,2-c]pyridin-4-yl]-3,4-dihydro-1H-isoquinoline-2-carboxylate (55 mg, 0.090 mmol) in THF (2 mL) was added Lawesson reagent (47 mg, 0.12 mmol). The reaction solution was stirred at 70° C. for 12 h. The reaction was cooled to rt and concentrated in vacuo. The residue was purified by preparative TLC (hexanes: ethyl acetate=1:1) to give tert-butyl 6-[6-carbamothioyl-7-[2,4-difluoro-6-(2-methoxyethoxy)phenyl]-3-fluoro-thieno[3,2-c]pyridin-4-yl]-3,4-dihydro-1H-isoquinoline-2-carboxylate (30 mg, 53% yield). LCMS ESI (+) m/z 630.2 (M+H).

Step L: Preparation of benzyl 2-[4-(2-tert-butoxycarbonyl-3,4-dihydro-1H-isoquinolin-6-yl)-7-[2,4-difluoro-6-(2-methoxyethoxy)phenyl]-3-fluoro-thieno[3,2-c]pyridin-6-yl]-6,7-dihydro-4H-thiazolo[5,4-c]pyridine-5-carboxylate: To a solution of tert-butyl 6-[6-carbamothioyl-7-[2,4-difluoro-6-(2-methoxyethoxy)phenyl]-3-fluoro-thieno[3,2-c]pyridin-4-yl]-3,4-dihydro-1H-isoquinoline-2-carboxylate (30 mg, 0.048 mmol) in ethanol (2 mL) was added benzyl 3-bromo-4-oxo-piperidine-1-carboxylate (37 mg, 0.12 mmol). The solution was then stirred at 70° C. for 4 h. The reaction solution was cooled to rt and concentrated in vacuo. The residue was purified by preparative TLC (hexane:ethyl acetate=1:1) to give benzyl 2-[4-(2-tert-butoxycarbonyl-3,4-dihydro-1H-isoquinolin-6-yl)-7-[2,4-difluoro-6-(2-methoxyethoxy)phenyl]-3-fluoro-thieno[3,2-c]pyridin-6-yl]-6,7-dihydro-4H-thiazolo[5,4-c]pyridine-5-carboxylate (28 mg, 70% yield).

Step M: Preparation of tert-butyl 6-[7-[2,4-difluoro-6-(2-methoxyethoxy)phenyl]-3-fluoro-6-(4,5,6,7-tetrahydrothiazolo[5,4-c]pyridin-2-yl)thieno[3,2-c]pyridin-4-yl]-3,4-dihydro-1H-isoquinoline-2-carboxylate: To a solution of benzyl 2-[4-(2-tert-butoxycarbonyl-3,4-dihydro-1H-isoquinolin-6-yl)-7-[2,4-difluoro-6-(2-methoxyethoxy)phenyl]-3-fluoro-thieno[3,2-c]pyridin-6-yl]-6,7-dihydro-4H-thiazolo[5,4-c]pyridine-5-carboxylate (28 mg, 0.033 mmol) in 1,4-dioxane (2 mL) and water (2 mL) was added lithium hydroxide (200 mg, 8.35 mmol). The mixture was stirred at 100° C. for 12 h. The mixture was diluted with water. The product was extracted with EtOAc, dried over anhydrous Na₂SO₄, filtered and concentrated to dryness to give crude tert-butyl 6-[7-[2,4-difluoro-6-(2-methoxyethoxy)phenyl]-3-fluoro-6-(4,5,6,7-tetrahydrothiazolo[5,4-c]pyridin-2-yl)thieno[3,2-c]pyridin-4-yl]-3,4-dihydro-1H-isoquinoline-2-carboxylate (30 mg, 96% yield) which was used in the next step directly. LCMS ESI (+) m/z 709.0 (M+H).

Step N: Preparation of tert-butyl 6-[7-[2,4-difluoro-6-(2-methoxyethoxy)phenyl]-3-fluoro-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate: To a solution of tert-butyl 6-[7-[2,4-difluoro-6-(2-methoxyethoxy)phenyl]-3-fluoro-6-(4,5,6,7-tetrahydrothiazolo[5,4-c]pyridin-2-yl)thieno[3,2-c]pyridin-4-yl]-3,4-dihydro-1H-isoquinoline-2-carboxylate (30 mg, 0.032 mmol) and triethylamine (0.015 mL, 0.11 mmol) in DCM (2 mL) was added acryloyl chloride (0.0054 mL, 0.066 mmol) at −60° C. The mixture was stirred at −60° C. for 10 min. The mixture was poured into NaHCO₃ aqueous solution and the product was extracted with DCM. The combined organic layers were washed with brine, dried over anhydrous Na₂SO₄, filtered and concentrated. The residue was purified by preparative TLC (EtOAc/petroleum ether: 1/1) to give tert-butyl 6-[7-[2,4-difluoro-6-(2-methoxyethoxy)phenyl]-3-fluoro-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (19 mg, 78% yield).

Step O: Preparation of 1-[2-[7-[2,4-difluoro-6-(2-methoxyethoxy)phenyl]-3-fluoro-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one: To a solution of tert-butyl 6-[7-[2,4-difluoro-6-(2-methoxyethoxy)phenyl]-3-fluoro-6-(5-prop-2-enoyl-6,7-dihydro-4H-thiazolo[5,4-c]pyridin-2-yl)thieno[3,2-c]pyridin-4-yl]-3,4-dihydro-1H-isoquinoline-2-carboxylate (19 mg, 0.025 mmol) in DCM (1 mL) was added trifluoroacetic acid (0.53 mL, 6.9 mmol). The mixture was stirred at 25° C. for 1 h. The mixture was concentrated and lyophilized to give 1-[2-[7-[2,4-difluoro-6-(2-methoxyethoxy)phenyl]-3-fluoro-4-(1,2,3,4-tetrahydroisoquinolin-6-yl)thieno[3,2-c]pyridin-6-yl]-6,7-dihydro-4H-thiazolo[5,4-c]pyridin-5-yl]prop-2-en-1-one (20 mg, 90% yield) as a bis trifluoroacetic acid salt. ¹H NMR (400 MHz, CD₃OD) δ 7.71-7.79 (m, 2H), 7.38-7.45 (m, 2H), 6.71-6.93 (m, 3H), 6.21-6.30 (m, 1H), 5.75-5.86 (m, 1H), 4.52 (s, 2H), 3.85-4.14 (m, 4H), 3.63 (t, J=6.4 Hz, 2H), 3.35-3.41 (m, 2H), 3.02 (s, 3H), 2.62-2.74 (m, 2H). LCMS ESI (+) m/z 662.9 (M+H).

### Synthetic Example 56: Synthesis of 5-[7-(2,4-difluoro-6-isopropoxy-phenyl)-6-(6-prop-2-enoyl-7,8-dihydro-5H-2,6-naphthyridin-3-yl)thieno[3,2-c]pyridin-4-yl]isoindolin-1-one (Compound 453)

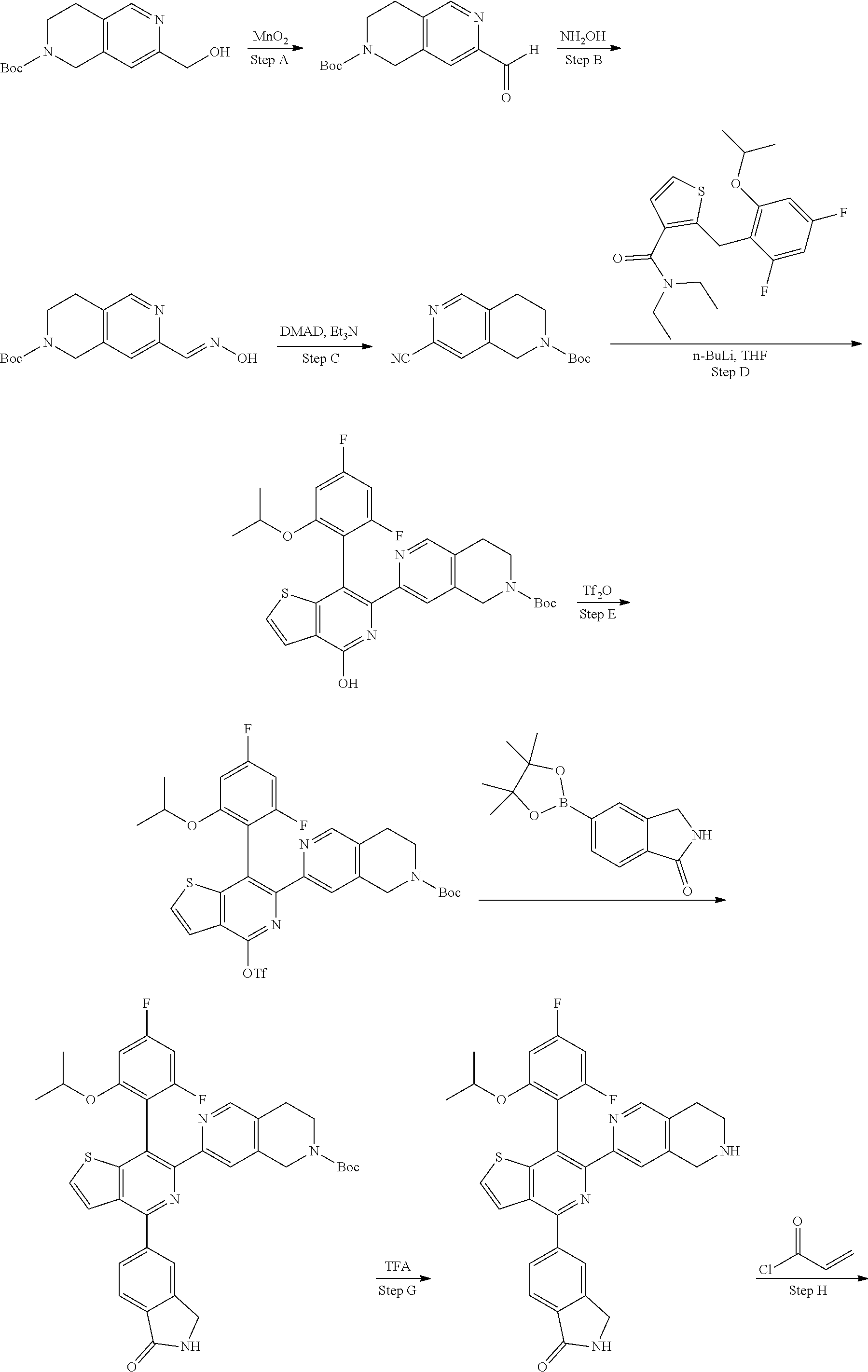

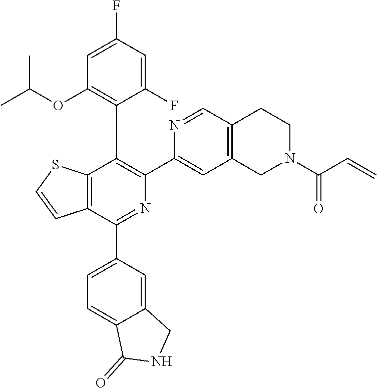 [SMILES: C=CC(=O)N1CCc2cnc(-c3nc(-c4ccc5c(c4)CNC5=O)c4ccsc4c3-c3c(F)cc(F)cc3OC(C)C)cc2C1]

Step A: Preparation of tert-butyl 7-formyl-3,4-dihydro-1H-2,6-naphthyridine-2-carboxylate: To a solution of tert-butyl 7-(hydroxymethyl)-3,4-dihydro-1H-2,6-naphthyridine-2-carboxylate (2.20 g, 8.32 mmol) in THF (10 mL) was added manganese dioxide (10.9 g, 125 mmol). The mixture was stirred at rt overnight. The mixture was filtered through celite and washed with THF. The filtrate was concentrated under vacuum to get tert-butyl 7-formyl-3,4-dihydro-1H-2,6-naphthyridine-2-carboxylate (2.10 g, 96% yield) as a pale brown solid. LCMS ESI (+) m/z 263.1 (M+H).

Step B: Preparation of tert-butyl 7-[(E)-hydroxyiminomethyl]-3,4-dihydro-1H-2,6-naphthyridine-2-carboxylate: To a solution of tert-butyl 7-formyl-3,4-dihydro-1H-2,6-naphthyridine-2-carboxylate (2.10 g, 8.01 mmol) and hydroxylamine hydrochloride (835 mg, 12.0 mmol) in ethanol (40 mL) and water (8 mL) was added sodium hydroxide (480 mg, 12.0 mmol) at 0° C. The mixture was stirred at rt overnight then diluted with water. The mixture was extracted with EtOAc. The combined organic layers were washed with brine, dried over sodium sulfate, concentrated and purified by flash column chromatography eluting with 3% MeOH in DCM to get tert-butyl 7-[(E)-hydroxyiminomethyl]-3,4-dihydro-1H-2,6-naphthyridine-2-carboxylate (2.20 g, 99% yield) as a white solid. LCMS ESI (+) m/z 278.2 (M+H).

Step C: Preparation of tert-butyl 7-cyano-3,4-dihydro-1H-2,6-naphthyridine-2-carboxylate: To a solution of tert-butyl 7-[(E)-hydroxyiminomethyl]-3,4-dihydro-1H-2,6-naphthyridine-2-carboxylate (2.20 g, 7.93 mmol) and triethylamine (803 mg, 7.93 mmol) in acetonitrile (40 mL) was added dimethyl acetylenedicarboxylate (2.00 mL, 15.9 mmol) at 0° C. The mixture was stirred at rt overnight then concentrated and purified by flash column chromatography eluting with 40% EtOAc in hexanes to afford tert-butyl 7-cyano-3,4-dihydro-1H-2,6-naphthyridine-2-carboxylate (1.40 g, 68% yield) as a white solid. LCMS ESI (+) m/z 260 (M+H).

Step D: Preparation of tert-butyl 7-[7-(2,4-difluoro-6-isopropoxy-phenyl)-4-hydroxy-thieno[3,2-c]pyridin-6-yl]-3,4-dihydro-1H-2,6-naphthyridine-2-carboxylate: To a solution of 2-[(2,4-difluoro-6-isopropoxy-phenyl)methyl]-N,N-diethyl-thiophene-3-carboxamide (697 mg, 1.90 mmol) in THF (8 mL) was added n-BuLi (2.4 M in hexanes, 0.78 mL, 1.88 mmol) at −60° C. under argon. The mixture was stirred at −60° C. for 30 min. Then tert-butyl 7-cyano-3,4-dihydro-1H-2,6-naphthyridine-2-carboxylate (400 mg, 1.54 mmol) in THF was added at −60° C. and the resulting mixture was stirred at ambient temperature for 2 hours. The reaction was quenched by slow addition of water. 1 N HCl was added to adjust the pH to 5-6. The mixture was extracted with EtOAc, dried over Na₂SO₄, filtered and concentrated. The residue was purified by silica gel column chromatography (DCM:MeOH=40:1) to give tert-butyl 7-[7-(2,4-difluoro-6-isopropoxy-phenyl)-4-hydroxy-thieno[3,2-c]pyridin-6-yl]-3,4-dihydro-1H-2,6-naphthyridine-2-carboxylate (250 mg, 29% yield) as a solid. LCMS ESI (+) m/z 553.8 (M+H).

Step E: Preparation of tert-butyl 7-[7-(2,4-difluoro-6-isopropoxy-phenyl)-4-(trifluoromethylsulfonyloxy)thieno[3,2-c]pyridin-6-yl]-3,4-dihydro-1H-2,6-naphthyridine-2-carboxylate: To a solution of tert-butyl 7-[7-(2,4-difluoro-6-isopropoxy-phenyl)-4-hydroxy-thieno[3,2-c]pyridin-6-yl]-3,4-dihydro-1H-2,6-naphthyridine-2-carboxylate (280 mg, 0.506 mmol) and pyridine (0.410 mL, 5.06 mmol) in DCM (3 mL) was added trifluoromethanesulfonic anhydride (285 mg, 1.01 mmol) at 0° C. The mixture was stirred at rt for 1 h and then diluted with water. The mixture was extracted with DCM. The combined organic layers were dried over Na₂SO₄, concentrated, and purified by column chromatography on silica gel (hexanes: EtOAc=5:1) to give tert-butyl 7-[7-(2,4-difluoro-6-isopropoxy-phenyl)-4-(trifluoromethylsulfonyloxy)thieno[3,2-c]pyridin-6-yl]-3,4-dihydro-1H-2,6-naphthyridine-2-carboxylate (230 mg, 66% yield). LCMS ESI (+) m/z 686.1 (M+H).

Step F: Preparation of tert-butyl 7-[7-(2,4-difluoro-6-isopropoxy-phenyl)-4-(1-oxoisoindolin-5-yl)thieno[3,2-c]pyridin-6-yl]-3,4-dihydro-1H-2,6-naphthyridine-2-carboxylate: A mixture of tert-butyl 7-[7-(2,4-difluoro-6-isopropoxy-phenyl)-4-(trifluoromethylsulfonyloxy)thieno[3,2-c]pyridin-6-yl]-3,4-dihydro-1H-2,6-naphthyridine-2-carboxylate (110 mg, 0.160 mmol), 5-(4,4,5,5-tetramethyl-1,3,2-dioxaborolan-2-yl)isoindolin-1-one (54 mg, 0.21 mmol), Pd(dppf)Cl₂ (12 mg, 0.016 mmol) and potassium acetate (31 mg, 0.32 mmol) in 1,4-dioxane (0.3 mL) and water (3 mL) was stirred at 70° C. under argon for 30 min. The reaction was concentrated to dryness. The crude was then purified by flash column chromatography eluting with 60% EtOAc in hexanes to afford tert-butyl 7-[7-(2,4-difluoro-6-isopropoxy-phenyl)-4-(1-oxoisoindolin-5-yl)thieno[3,2-c]pyridin-6-yl]-3,4-dihydro-1H-2,6-naphthyridine-2-carboxylate (90 mg, 84% yield). LCMS ESI (+) m/z 669.2 (M+H).

Step G: Preparation of 5-[7-(2,4-difluoro-6-isopropoxy-phenyl)-6-(5,6,7,8-tetrahydro-2,6-naphthyridin-3-yl)thieno[3,2-c]pyridin-4-yl]isoindolin-1-one: To a solution of tert-butyl 7-[7-(2,4-difluoro-6-isopropoxy-phenyl)-4-(1-oxoisoindolin-5-yl)thieno[3,2-c]pyridin-6-yl]-3,4-dihydro-1H-2,6-naphthyridine-2-carboxylate (90 mg, 0.14 mmol) in DCM (2.5 mL) was added TFA (1 mL) at 0° C. The mixture was stirred at 25° C. for 1 h then concentrated to get 5-[7-(2,4-difluoro-6-isopropoxy-phenyl)-6-(5,6,7,8-tetrahydro-2,6-naphthyridin-3-yl)thieno[3,2-c]pyridin-4-yl]isoindolin-1-one (90 mg, crude) as a brown solid. The crude product was used in next step without any further purification. LCMS ESI (+) m/z 569.2 (M+H).

Step H: Preparation of 5-[7-(2,4-difluoro-6-isopropoxy-phenyl)-6-(6-prop-2-enoyl-7,8-dihydro-5H-2,6-naphthyridin-3-yl)thieno[3,2-c]pyridin-4-yl]isoindolin-1-one: To a solution of 5-[7-(2,4-difluoro-6-isopropoxy-phenyl)-6-(5,6,7,8-tetrahydro-2,6-naphthyridin-3-yl)thieno[3,2-c]pyridin-4-yl]isoindolin-1-one (90 mg, 0.16 mmol) and DIEA (0.28 mL, 1.6 mmol) in DCM (4 mL) was added acryloyl chloride (0.019 mL, 0.24 mmol) at −60° C. The mixture was stirred at −60° C. for 15 min. The reaction was quenched with aqueous NaHCO₃ solution. The organics were then separated and dried (MgSO₄) before concentration to dryness. The crude was purified by flash column chromatography on silica gel eluting with 4% MeOH in DCM followed by preparative HPLC (column: Gemini-C18 150×21.2 mm, 5 μm; mobile phase: ACN-H₂O (0.1% TFA); gradient: 20-50%) to afford 5-[7-(2,4-difluoro-6-isopropoxy-phenyl)-6-(6-prop-2-enoyl-7,8-dihydro-5H-2,6-naphthyridin-3-yl)thieno[3,2-c]pyridin-4-yl]isoindolin-1-one (9.0 mg, 9% yield). ¹H NMR (400 MHz, CD₃OD) δ 8.29-8.17 (m, 2H), 8.12 (d, J=7.9 Hz, 1H), 7.97 (d, J=7.9 Hz, 1H), 7.80 (d, J=5.5 Hz, 1H), 7.70 (d, J=5.5 Hz, 1H), 7.53 (d, J=26.0 Hz, 1H), 6.93-6.73 (m, 1H), 6.68 (t, J=10.3 Hz, 1H), 6.62-6.54 (m, 1H), 6.27-6.16 (m, 1H), 5.77 (d, J=10.6 Hz, 1H), 4.74 (d, J=29.0 Hz, 2H), 4.59 (s, 2H), 4.55-4.41 (m, 1H), 3.86 (t, J=5.8 Hz, 2H), 2.95-2.80 (m, 2H), 1.07 (t, J=6.0 Hz, 3H), 0.92 (dd, J=22.4, 5.8 Hz, 3H). LCMS ESI (+) m/z 623.2 (M+H).

Step I: Preparation of tert-butyl (4S,7S)-2-[7-[2,4-difluoro-6-(2-methoxyethoxy) phenyl]-4-hydroxy-thieno[3,2-c]pyridin-6-yl]-4,7-dimethyl-6,7-dihydro-4H-pyrazolo[1,5-a]pyrazine-5-carboxylate: To a solution of 2-[[2,4-difluoro-6-(2-methoxyethoxy)phenyl]methyl]-N,N-diethyl-thiophene-3-carboxamide (200 mg, 0.521 mmol) in THF (3 mL) was added n-BuLi (2.5 M in hexanes, 0.22 mL, 0.55 mmol) at −70° C. under Ar atmosphere. The mixture was stirred at −70° C. for 30 minutes. A solution of tert-butyl (4S,7S)-2-cyano-4,7-dimethyl-6,7-dihydro-4H-pyrazolo[1,5-a]pyrazine-5-carboxylate (120 mg, 0.434 mmol) in THF (0.5 mL) was added. The resulting mixture was stirred room temperature for 2 hours. The reaction mixture was poured into saturated aqueous NH₄Cl solution and extracted with EtOAc. The organic phase was dried over anhydrous Na₂SO₄, filtered and concentrated to afford crude tert-butyl (4S,7S)-2-[7-[2,4-difluoro-6-(2-methoxyethoxy)phenyl]-4-hydroxy-thieno[3,2-c]pyridin-6-yl]-4,7-dimethyl-6,7-dihydro-4H-pyrazolo[1,5-a]pyrazine-5-carboxylate (220 mg, 86% yield) as a yellow solid. LCMS ESI (+) m/z 587.0 (M+H).

### Synthetic Example 57: Synthesis of 1-((R)-2-((R)-7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazin-5(4H)-yl)prop-2-en-1-one (Compound 456) and 1-((R)-2-((S)-7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazin-5(4H)-yl)prop-2-en-1-one (Compound 457)

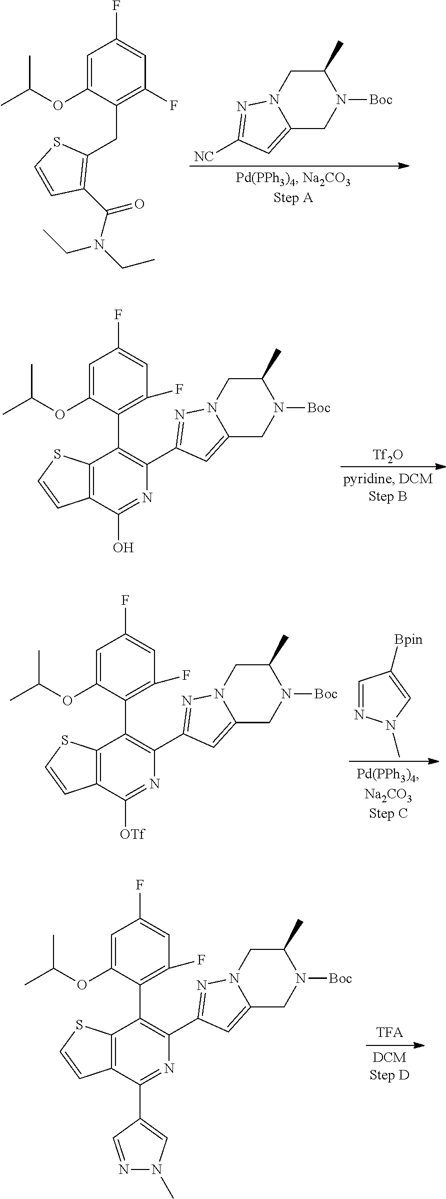

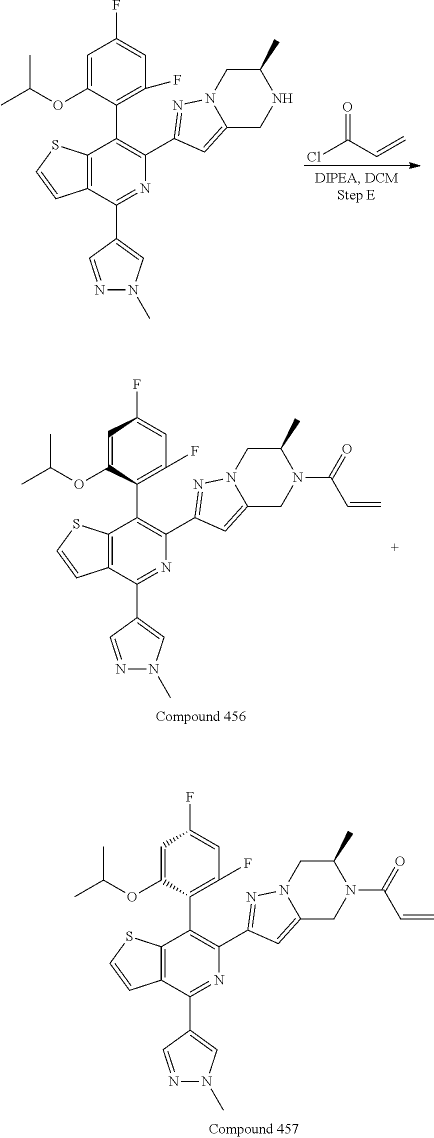

Step A: Preparation of tert-butyl (6R)-2-(7-(2,4-difluoro-6-isopropoxyphenyl)-4-hydroxythieno[3,2-c]pyridin-6-yl)-6-methyl-6,7-dihydropyrazolo[1,5-a]pyrazine-5(4H)-carboxylate: To a stirred solution of 2-[(2,4-difluoro-6-isopropoxy-phenyl)methyl]-N,N-diethyl-thiophene-3-carboxamide (790 mg, 2.15 mmol) in THF (8 mL) at −78° C. was added n-butyllithium (2.5 M in hexanes, 0.932 mL, 2.33 mmol) dropwise. The mixture was stirred at −78° C. for 30 min. tert-Butyl-(6R)-2-cyano-6-methyl-6,7-dihydro-4H-pyrazolo[1,5-a]pyrazine-5-carboxylate (470 mg, 1.79 mmol) in THF (3 mL) was added, and the resulting mixture was stirred at rt for 12 hours. The mixture was diluted with water, and 1 N HCl was added to adjust the pH to 5-6. The mixture was extracted with EtOAc, dried and concentrated. The residue was purified by silica gel column (petroleum ether:EtOAc=5:1 to DCM:MeOH=10:1) to give tert-butyl (6R)-2-(7-(2,4-difluoro-6-isopropoxyphenyl)-4-hydroxythieno[3,2-c]pyridin-6-yl)-6-methyl-6,7-dihydropyrazolo[1,5-a]pyrazine-5(4H)-carboxylate (900 mg, 90% yield). LCMS ESI (+) m/z 557.0 (M+H).

Step B: Preparation of tert-butyl (6R)-2-(7-(2,4-difluoro-6-isopropoxyphenyl)-4-(((trifluoromethyl)sulfonyl)oxy)thieno[3,2-c]pyridin-6-yl)-6-methyl-6,7-dihydropyrazolo[1,5-a]pyrazine-5(4H)-carboxylate: To a solution of tert-butyl (6R)-2-(7-(2,4-difluoro-6-isopropoxyphenyl)-4-hydroxythieno[3,2-c]pyridin-6-yl)-6-methyl-6,7-dihydropyrazolo[1,5-a]pyrazine-5(4H)-carboxylate (900 mg, 1.62 mmol) and pyridine (0.880 mL, 8.08 mmol) in DCM (30 mL) was added trifluoromethanesulfonic anhydride (0.820 mL, 4.85 mmol) at 0° C. The mixture was stirred at 20° C. for 30 min. Aqueous saturated NaHCO₃ solution was added to quench the reaction. The product was extracted with DCM, dried over Na₂SO₄ and concentrated. The residue was purified by silica gel column (petroleum ether:EtOAc=4:1) to give tert-butyl (6R)-2-(7-(2,4-difluoro-6-isopropoxyphenyl)-4-(((trifluoromethyl)sulfonyl)oxy) thieno[3,2-c]pyridin-6-yl)-6-methyl-6,7-dihydropyrazolo[1,5-a]pyrazine-5(4H)-carboxylate (830 mg, 75% yield). LCMS ESI (+) m/z 689.2 (M+H).

Step C: Preparation of tert-butyl (6R)-2-(7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazine-5(4H)-carboxylate: A mixture of tert-butyl (6R)-2-(7-(2,4-difluoro-6-isopropoxyphenyl)-4-(((trifluoromethyl)sulfonyl)oxy)thieno[3,2-c]pyridin-6-yl)-6-methyl-6,7-dihydropyrazolo[1,5-a]pyrazine-5(4H)-carboxylate (400 mg, 0.581 mmol), 1-methyl-4-(4,4,5,5-tetramethyl-1,3,2-dioxaborolan-2-yl)pyrazole (242 mg, 1.16 mmol), tetrakis(triphenylphosphine)palladium(0) (67 mg, 0.058 mmol) and sodium carbonate (123 mg, 1.16 mmol) in 1,4-dioxane (8 mL)/water (0.80 mL) was stirred at 95° C. for 2 hours under N₂. After cooling, the mixture was concentrated and purified by preparative HPLC to give tert-butyl (6R)-2-(7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazine-5(4H)-carboxylate (286 mg, 79% yield). LCMS ESI (+) m/z 621.0 (M+H).

Step D: Preparation of 7-(2,4-difluoro-6-isopropoxyphenyl)-4-(1-methyl-1H-pyrazol-4-yl)-6-((R)-6-methyl-4,5,6,7-tetrahydropyrazolo[1,5-a]pyrazin-2-yl)thieno[3,2-c]pyridine: To a solution of tert-butyl (6R)-2-(7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazine-5(4H)-carboxylate (286 mg, 0.461 mmol) in DCM (6 mL) was added trifluoroacetic acid (1.5 mL) at 20° C. The mixture was stirred at 20° C. for 30 min. The mixture was concentrated to dryness to get crude 7-(2,4-difluoro-6-isopropoxyphenyl)-4-(1-methyl-1H-pyrazol-4-yl)-6-((R)-6-methyl-4,5,6,7-tetrahydropyrazolo[1,5-a]pyrazin-2-yl)thieno[3,2-c]pyridine (300 mg) which was used in the next step directly. LCMS ESI (+) m/z 521.0 (M+H).

Step E: Preparation of 1-((R)-2-((R)-7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazin-5(4H)-yl)prop-2-en-1-one (Compound 456) and 1-((R)-2-((S)-7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazin-5(4H)-yl)prop-2-en-1-one (Compound 457): To a stirred solution of 7-(2,4-difluoro-6-isopropoxyphenyl)-4-(1-methyl-1H-pyrazol-4-yl)-6-((R)-6-methyl-4,5,6,7-tetrahydropyrazolo[1,5-a]pyrazin-2-yl)thieno[3,2-c]pyridine (300 mg, 0.461 mmol) and DIPEA (0.510 mL, 2.88 mmol) in DCM (2 mL) was added acryloyl chloride (50.0 mg, 0.553 mmol) at 0° C. The mixture was stirred at 0° C. for 30 min. Saturated NaHCO₃ aqueous solution was added to quench the reaction, and the product was extracted with DCM. The organic phase was dried over Na₂SO₄, filtered and concentrated. The residue was purified by preparative TLC (DCM:MeOH=10:1) to give 1-((6R)-2-(7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazin-5(4H)-yl)prop-2-en-1-one (239 mg, 71% yield) as a mixture of two diastereomers. 230 mg of the mixture was separated by chiral SFC (CHIRAL ART Cellulose-SB, 3 cm×25 cm, 5 μm, CO₂:MeOH (containing 0.1% 2 mM NH₃-MeOH)=55:45) to give 1-((R)-2-((R)-7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazin-5(4H)-yl)prop-2-en-1-one (Compound 456) (79.6 mg) as the first peak, and 1-((R)-2-((S)-7-(2,4-difluoro-6-isopropoxyphenyl)-4-(1-methyl-1H-pyrazol-4-yl)thieno[3,2-c]pyridin-6-yl)-6-methyl-6,7-dihydropyrazolo[1,5-a]pyrazin-5(4H)-yl)prop-2-en-1-one (Compound 457) (104.1 mg) as the second peak. Compound 456: ¹H NMR (400 MHz, CD₃OD) δ 8.23 (s, 1H), 8.15 (s, 1H), 7.75 (d, J=5.7 Hz, 1H), 7.49 (d, J=5.4 Hz, 1H), 6.49-6.59 (m, 3H), 6.35 (d, J=18.6 Hz, 1H), 6.11 (s, 1H), 5.77 (d, J=12.3 Hz, 1H), 5.15 (s, 1H), 4.40 (s, 1H), 4.32-4.38 (m, 2H), 4.13-4.22 (m, 3H), 4.02 (s, 3H), 1.20 (d, J=6.9 Hz, 3H), 1.08 (d, J=6.0 Hz, 3H), 0.93 (d, J=6.0 Hz, 3H); LCMS ESI (+) m/z 575.2 (M+H). Compound 457: ¹H NMR (400 MHz, CD₃OD) δ 8.27 (s, 1H), 8.14 (s, 1H), 7.75 (d, J=5.4 Hz, 1H), 7.50 (d, J=5.4 Hz, 1H), 6.50-6.63 (m, 3H), 6.35 (d, J=18.6 Hz, 1H), 6.08 (s, 1H), 5.77 (dd, J=1.8, 10.5 Hz, 1H), 4.8-5.2 (m, 2H), 4.25-4.43 (m, 2H), 4.12-4.22 (m, 2H), 4.02 (s, 3H), 1.21 (d, J=6.6 Hz, 3H), 1.09 (d, J=6.0 Hz, 3H), 0.95 (d, J=6.0 Hz, 3H); LCMS ESI (+) m/z 575.2 (M+H).

### Synthetic Example 58: Synthesis of 1-((7R)-3-(7-(2,4-difluoro-6-isopropoxyphenyl)-4-(2-methyl-1,2,3,4-tetrahydroisoquinolin-6-yl)thieno[3,2-c]pyridin-6-yl)-7-methyl-7,8-dihydro-1,6-naphthyridin-6(5H)-yl)prop-2-en-1-one (Compound 476)

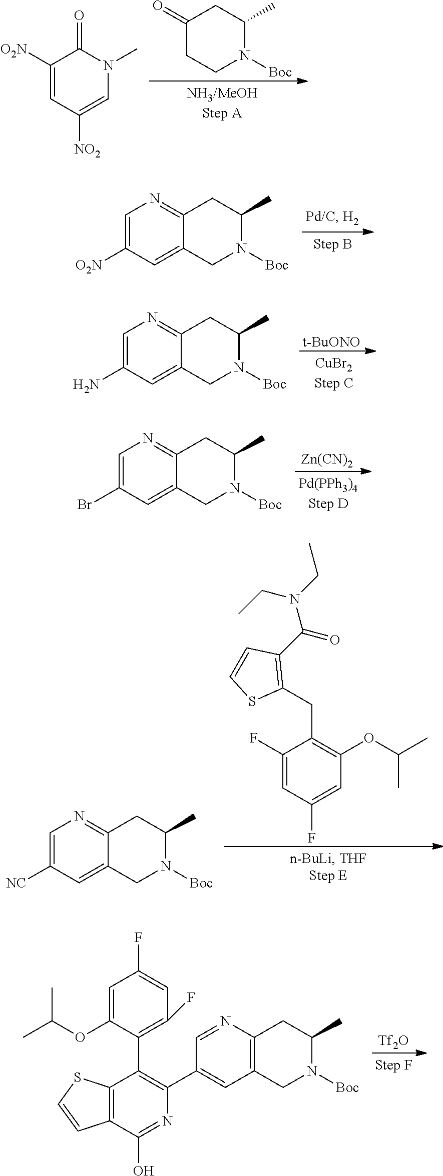

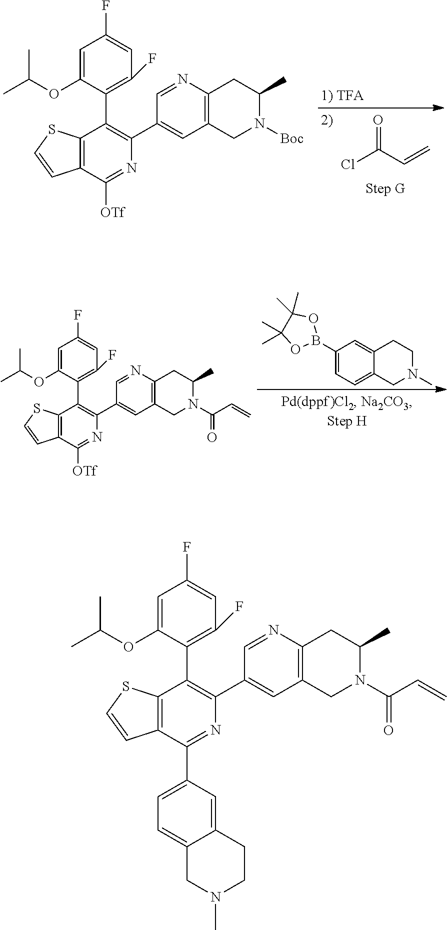

Step A: Preparation of tert-butyl (R)-7-methyl-3-nitro-7,8-dihydro-1,6-naphthyridine-6(5H)-carboxylate: A mixture of 1-methyl-3,5-dinitro-pyridin-2-one (5.00 g, 25.1 mmol), tert-butyl (2R)-2-methyl-4-oxo-piperidine-1-carboxylate (5.36 g, 25.1 mmol) and 7 N ammonia in MeOH (36.0 mL, 251 mmol) in methanol (35 mL) was stirred at 60° C. for 4 h. Upon completion of the reaction, the resulting mixture was concentrated to give of tert-butyl (R)-7-methyl-3-nitro-7,8-dihydro-1,6-naphthyridine-6(5H)-carboxylate (6.60 g, 90% yield) as a light-yellow solid. LCMS ESI (+) m/z 294.0 (M+H).

Step B: Preparation of tert-butyl (R)-3-amino-7-methyl-7,8-dihydro-1,6-naphthyridine-6(5H)-carboxylate: A mixture of tert-butyl (7R)-7-methyl-3-nitro-7,8-dihydro-5H-1,6-naphthyridine-6-carboxylate (7.00 g, 23.9 mmol) and Pd/C (0.510 g, 4.77 mmol) in methanol (150 mL) was stirred at rt under H₂ for 12 h. Upon completion of the reaction, the resulting mixture was filtered. The filtrate was concentrated to give tert-butyl (R)-3-amino-7-methyl-7,8-dihydro-1,6-naphthyridine-6(5H)-carboxylate (6.20 g, 89% yield). LCMS ESI (+) m/z 264.0 (M+H).

Step C: Preparation of tert-butyl (R)-3-bromo-7-methyl-7,8-dihydro-1,6-naphthyridine-6(5H)-carboxylate: To a stirred solution of tert-butyl (7R)-3-amino-7-methyl-7,8-dihydro-5H-1,6-naphthyridine-6-carboxylate (6.20 g, 23.5 mmol) and CuBr₂ (7.89 g, 35.3 mmol) in MeCN (15 mL) was added tert-butyl nitrite (3.64 g, 35.3 mmol) at 0° C. The resulting mixture was stirred at rt for 12 h. Upon completion of the reaction, saturated aqueous Na₂S₂O₃ solution (100 mL) was added. The resulting mixture was filtered. The filtrate was poured into water and extracted with EtOAc. The organic layers were combined, dried over MgSO₄ and concentrated under reduced pressure. The residue was purified by flash column chromatography on silica gel (EtOAc/petroleum ether=0 to 30%) to give tert-butyl (R)-3-bromo-7-methyl-7,8-dihydro-1,6-naphthyridine-6(5H)-carboxylate (6.50 g, 80%) as a brown oil. LCMS ESI (+) m/z 327.0 (M+H).

Step D: Preparation of tert-butyl (R)-3-cyano-7-methyl-7,8-dihydro-1,6-naphthyridine-6(5H)-carboxylate: A mixture of tert-butyl (7R)-3-bromo-7-methyl-7,8-dihydro-5H-1,6-naphthyridine-6-carboxylate (6.50 g, 19.9 mmol), Zn(CN)₂ (4.66 g, 39.7 mmol) and Pd(PPh₃)₄(2.29 g, 1.99 mmol) in DMA (100 mL) was stirred at 120° C. under N₂ for 12 h. Upon completion of the reaction, the mixture was diluted with water. The aqueous layer was separated and extracted with EtOAc. The organic layers were combined, dried over MgSO₄ and concentrated under reduced pressure. The residue was purified by flash chromatography (petroleum ether/EtOAc=0-30%) to give tert-butyl (R)-3-cyano-7-methyl-7,8-dihydro-1,6-naphthyridine-6(5H)-carboxylate (4.50 g, 80% yield) as a white solid. LCMS ESI (+) m/z 273.9 (M+H).

Step E to Step H: Preparation of 1-((7R)-3-(7-(2,4-difluoro-6-isopropoxyphenyl)-4-(2-methyl-1,2,3,4-tetrahydroisoquinolin-6-yl)thieno[3,2-c]pyridin-6-yl)-7-methyl-7,8-dihydro-1,6-naphthyridin-6(5H)-yl)prop-2-en-1-one: The title compound was prepared as described in Synthetic Example 54 Step C to Step G, substituting tert-butyl (R)-3-cyano-7-methyl-7,8-dihydro-1,6-naphthyridine-6(5H)-carboxylate for tert-butyl 2-cyano-6,8-dihydro-5H-1,7-naphthyridine-7-carboxylate in Step C, and substituting 2-methyl-6-(4,4,5,5-tetramethyl-1,3,2-dioxaborolan-2-yl)-3,4-dihydro-1H-isoquinoline for 5-(4,4,5,5-tetramethyl-1,3,2-dioxaborolan-2-yl)isoindolin-1-one in Step G. The crude was purified by flash column chromatography on silica gel eluting with 0-10% MeOH in DCM followed by preparative HPLC (column: Gemini-C18 150×21.2 mm, 5 μm; mobile phase: ACN-H₂O (0.1% TFA); gradient: 20-40%) to afford 1-((7R)-3-(7-(2,4-difluoro-6-isopropoxyphenyl)-4-(2-methyl-1,2,3,4-tetrahydroisoquinolin-6-yl)thieno[3,2-c]pyridin-6-yl)-7-methyl-7,8-dihydro-1,6-naphthyridin-6(5H)-yl)prop-2-en-1-one as a trifluoroacetic acid salt. ¹H NMR (400 MHz, CD₃OD) δ 8.74-8.46 (m, 1H), 8.14-7.96 (m, 1H), 7.96-7.82 (m, 3H), 7.75 (t, J=5.7 Hz, 1H), 7.48 (d, J=8.5 Hz, 1H), 6.99-6.78 (m, 2H), 6.75-6.62 (m, 1H), 6.28 (d, J=16.6 Hz, 1H), 5.83 (d, J=10.7 Hz, 1H), 5.45-5.23 (m, 1H), 4.84-4.23 (m, 5H), 3.95-3.77 (m, 1H), 3.61-2.94 (m, 4H), 3.21-2.90 (m, 4H), 1.49-0.91 (m, 9H). LCMS ESI (+) m/z 651.3 (M+H).

### Synthetic Example 59: Synthesis of 1-((R)-2-((R)-7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazin-5(4H)-yl)prop-2-en-1-one (Compound 478) and 1-((R)-2-((S)-7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazin-5(4H)-yl)prop-2-en-1-one (Compound 479)

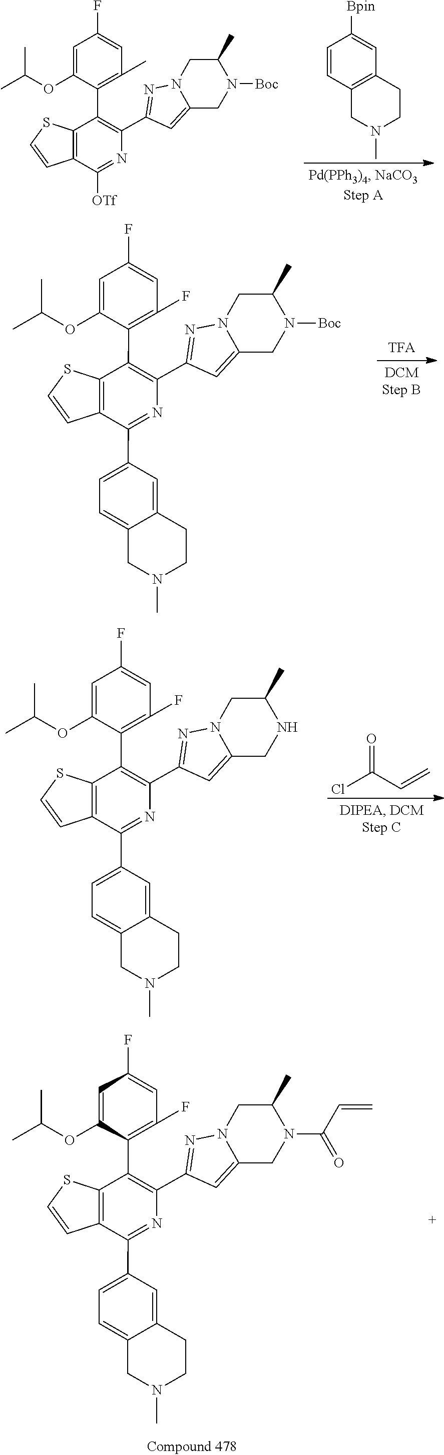

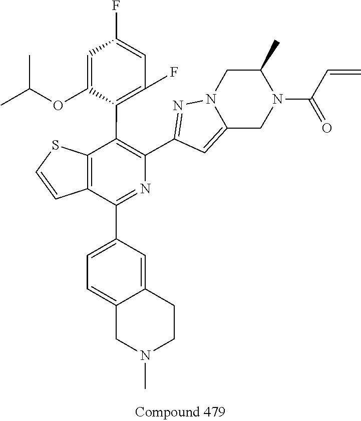

Step A: Preparation of tert-butyl (6R)-2-(7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazine-5(4H)-carboxylate: A mixture of tert-butyl (6R)-2-(7-(2,4-difluoro-6-isopropoxyphenyl)-4-(((trifluoromethyl)sulfonyl)oxy)thieno[3,2-c]pyridin-6-yl)-6-methyl-6,7-dihydropyrazolo[1,5-a]pyrazine-5(4H)-carboxylate (200 mg, 0.290 mmol), 2-methyl-6-(4,4,5,5-tetramethyl-1,3,2-dioxaborolan-2-yl)-3,4-dihydro-1H-isoquinoline (238 mg, 0.871 mmol), tetrakis(triphenylphosphine)palladium(0) (34 mg, 0.029 mmol) and sodium carbonate (185 mg, 1.74 mmol) in 1,4-dioxane (10 mL)/water (1 mL) was stirred at 95° C. for 2 hours under N₂. The mixture was concentrated and purified by silica gel column (EtOAc to DCM:MeOH=10:1) to give tert-butyl (6R)-2-(7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazine-5(4H)-carboxylate (95 mg, 48% yield). LCMS ESI (+) m/z 686.0 (M+H).

Step B: Preparation of 7-(2,4-difluoro-6-isopropoxyphenyl)-4-(2-methyl-1,2,3,4-tetrahydroisoquinolin-6-yl)-6-((R)-6-methyl-4,5,6,7-tetrahydropyrazolo[1,5-a]pyrazin-2-yl)thieno[3,2-c]pyridine: To a solution of tert-butyl (6R)-2-(7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazine-5(4H)-carboxylate (95 mg, 0.14 mmol) in DCM (4 mL) was added trifluoroacetic acid (2.8 mL) at 20° C. The mixture was stirred at 20° C. for 30 min. The mixture was concentrated to dryness to give crude 7-(2,4-difluoro-6-isopropoxyphenyl)-4-(2-methyl-1,2,3,4-tetrahydroisoquinolin-6-yl)-6-((R)-6-methyl-4,5,6,7-tetrahydropyrazolo[1,5-a]pyrazin-2-yl)thieno[3,2-c]pyridine (120 mg, 100% yield) which was used in the next step without further purification. LCMS ESI (+) m/z 586.0 (M+H).

Step C: Preparation of 1-((R)-2-((R)-7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazin-5(4H)-yl)prop-2-en-1-one (Compound 478) and 1-((R)-2-((S)-7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazin-5(4H)-yl)prop-2-en-1-one (Compound 479): To a stirred solution of 7-(2,4-difluoro-6-isopropoxyphenyl)-4-(2-methyl-1,2,3,4-tetrahydroisoquinolin-6-yl)-6-((R)-6-methyl-4,5,6,7-tetrahydropyrazolo[1,5-a]pyrazin-2-yl)thieno[3,2-c]pyridine (43 mg, 0.073 mmol) and DIPEA (0.039 mL, 0.22 mmol) in DCM (2 mL) was added acryloyl chloride (0.0072 mL, 0.088 mmol) at 0° C. The mixture was stirred at 0° C. for 30 min. Saturated NaHCO₃ aqueous solution was added to quench the reaction, and the product was extracted with DCM. The organic phase was dried over Na₂SO₄, filtered and concentrated. The residue was purified by preparative HPLC to give 1-((6R)-2-(7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazin-5(4H)-yl)prop-2-en-1-one (17 mg, 35% yield) as a mixture of two diastereomers. 98.9 mg of the mixture was separated by chiral SFC (CHIRAL ART Cellulose-SB, 3 cm×25 cm, 5 μm, CO₂:[MeOH:DCM=2:1(0.1% 2 mM NH₃-MeOH)]=75:25) to give 1-((R)-2-((R)-7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazin-5(4H)-yl)prop-2-en-1-one (Compound 478) (29.0 mg) as the first peak, and 1-((R)-2-((S)-7-(2,4-difluoro-6-isopropoxyphenyl)-4-(2-methyl-1,2,3,4-tetrahydroisoquinolin-6-yl)thieno[3,2-c]pyridin-6-yl)-6-methyl-6,7-dihydropyrazolo[1,5-a]pyrazin-5(4H)-yl)prop-2-en-1-one (Compound 479) (31.8 mg) as the second peak. Compound 478: ¹H NMR (400 MHz, CD₃OD) δ 7.80 (s, 1H), 7.76 (d, J=7.8 Hz, 1H), 7.62 (d, J=5.7 Hz, 1H), 7.44 (d, J=5.7 Hz, 1H), 7.19 (d, J=8.1 Hz, 1H), 6.51-6.59 (m, 3H), 6.35 (d, J=18.3 Hz, 1H), 6.16 (s, 1H), 5.77 (dd, J=1.8, 10.5 Hz, 1H), 4.33-4.41 (m, 1H), 4.11-4.22 (m, 2H), 3.85 (s, 2H), 3.13 (t, J=5.7 Hz, 2H), 2.94 (t, J=5.7 Hz, 2H), 2.61 (s, 3H), 1.17 (d, J=9.3 Hz, 3H), 1.10 (d, J=6.0 Hz, 3H), 0.95 (d, J=6.0 Hz, 3H); LCMS ESI (+) m/z 640.4 (M+H). Compound 479: ¹H NMR (400 MHz, CD₃OD) δ 7.80 (s, 1H), 7.76 (d, J=7.8 Hz, 1H), 7.62 (d, J=5.4 Hz, 1H), 7.44 (d, J=5.4 Hz, 1H), 7.19 (d, J=7.8 Hz, 1H), 7.51-7.57 (m, 3H), 6.37 (d, J=18.3 Hz, 1H), 6.14 (s, 1H), 5.77 (dd, J=1.8, 10.5 Hz, 1H), 4.34-4.42 (m, 1H), 4.11-4.22 (m, 2H), 3.85 (s, 2H), 3.12 (t, J=5.7 Hz, 2H), 2.93 (s, 2H), 2.61 (s, 3H), 1.20 (d, J=6.6 Hz, 3H), 1.00 (d, J=6.0 Hz, 3H), 0.95 (d, J=6.0 Hz, 3H); LCMS ESI (+) m/z 640.3 (M+H).

### Synthetic Example 60: Synthesis of 1-((4S,6R)-2-(7-(2,4-difluoro-6-(2-methoxyethoxy)phenyl)-4-(2-methyl-1,2,3,4-tetrahydroisoquinolin-6-yl)thieno[3,2-c]pyridin-6-yl)-4,6-dimethyl-6,7-dihydropyrazolo[1,5-a]pyrazin-5(4H)-yl)prop-2-en-1-one (Compound 480)

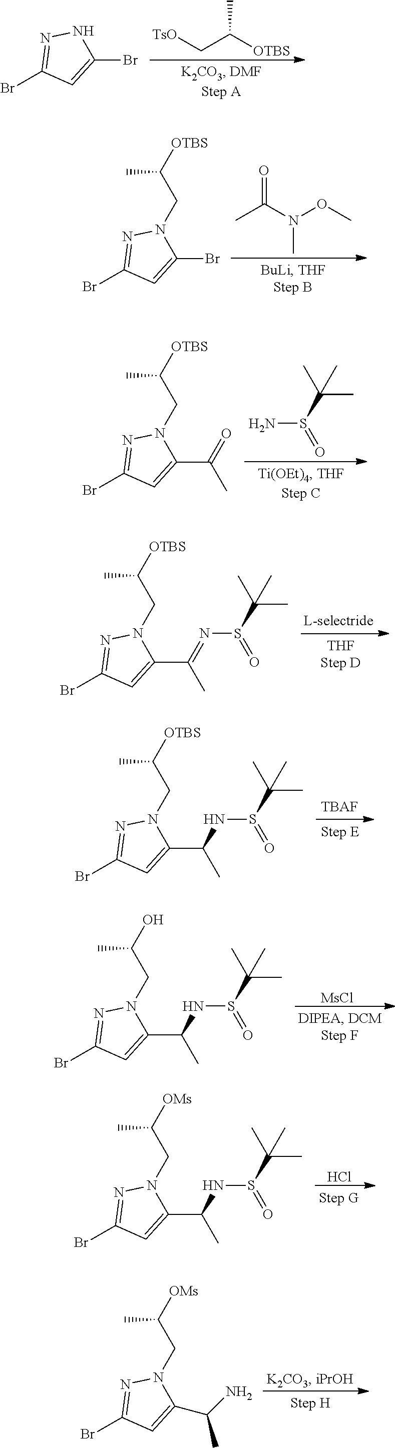

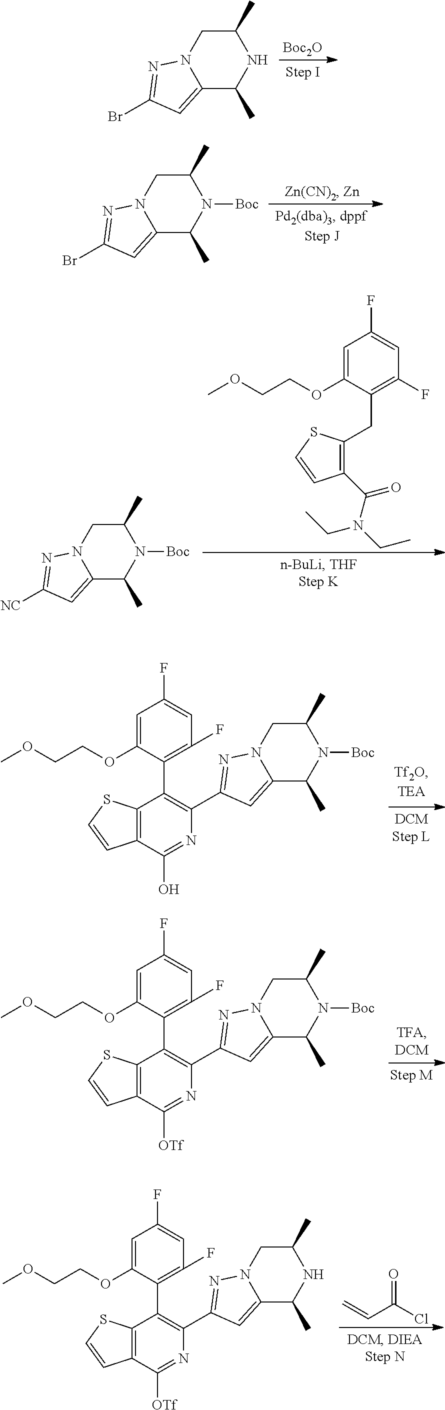

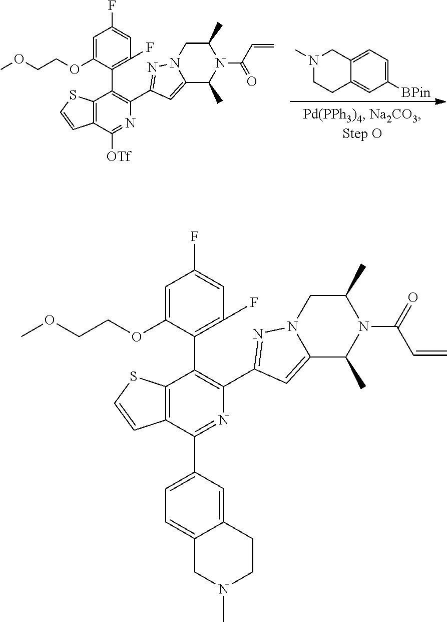

Step A: Preparation of (S)-3,5-dibromo-1-(2-((tert-butyldimethylsilyl)oxy)propyl)-1H-pyrazole: A mixture of [(2S)-2-[tert-butyl(dimethyl)silyl]oxypropyl]4-methylbenzenesulfonate (11.5 g, 33.4 mmol), K₂CO₃ (13.8 g, 100 mmol) and 3,5-dibromo-1H-pyrazole (6.03 g, 26.7 mmol) in DMF (60 mL) was stirred at 50° C. for 16 h. The mixture was diluted with EtOAc, washed with water and brine, dried over Na₂SO₄, filtered and concentrated. The residue was purified by column chromatography on silica gel (petroleum ether/EtOAc=20:1→5:1) to give (S)-3,5-dibromo-1-(2-((tert-butyldimethylsilyl)oxy)propyl)-1H-pyrazole (8.50 g, 64% yield) as a light yellow oil.

Step B: Preparation of (S)-1-(3-bromo-1-(2-((tert-butyldimethylsilyl)oxy)propyl)-1H-pyrazol-5-yl)ethan-1-one: To a solution of (S)-3,5-dibromo-1-(2-((tert-butyldimethylsilyl)oxy)propyl)-1H-pyrazole (8.00 g, 20.1 mmol) in THF (80 mL) was added n-butyllithium (2.5 M in hexanes, 9.64 mL, 24.1 mmol) dropwise at −78° C. After stirring for 30 min, N-methoxy-N-methyl-acetamide (4.14 g, 40.2 mmol) in THF (10 mL) was added to the mixture at −78° C. After stirring for 30 min, the reaction was warmed to rt and stir overnight. The reaction was taken up with EtOAc, washed with brine, dried over Na₂SO₄, filtered and concentrated. The residue was purified by column chromatography on silica gel eluting with (petroleum ether/EtOAc=20:1-3:1) to give (S)-1-(3-bromo-1-(2-((tert-butyldimethylsilyl)oxy)propyl)-1H-pyrazol-5-yl)ethan-1-one (5.80 g, 24% yield) as a light yellow oil. LCMS ESI (+) m/z 361.0 (M+H).

Step C: Preparation of (R)—N—((E)-1-(3-bromo-1-((S)-2-((tert-butyldimethylsilyl)oxy)propyl)-1H-pyrazol-5-yl)ethylidene)-2-methylpropane-2-sulfinamide: A mixture of S)-1-(3-bromo-1-(2-((tert-butyldimethylsilyl)oxy)propyl)-1H-pyrazol-5-yl)ethan-1-one (5.80 g, 4.82 mmol), (R)-2-methylpropane-2-sulfinamide (1.17 g, 9.63 mmol) and titanium ethoxide (2.20 g, 9.63 mmol) in THF (50 mL) was stirred at 80° C. for 16 h. After cooling to rt, the reaction was quenched with water. The mixture was filtered through a Celite pad, and the filtrate was concentrated. The crude was purified by column chromatography on silica gel eluting with (petroleum ether/EtOAc 20:1-1:1) to give (R)—N—((E)-1-(3-bromo-1-((S)-2-((tert-butyldimethylsilyl)oxy)propyl)-1H-pyrazol-5-yl)ethylidene)-2-methylpropane-2-sulfinamide (1.60 g, 72% yield) as a light yellow solid.

Step D: Preparation of (R)—N—((S)-1-(3-bromo-1-((S)-2-((tert-butyldimethylsilyl)oxy)propyl)-1H-pyrazol-5-yl)ethyl)-2-methylpropane-2-sulfinamide: To a solution of (R)—N—((E)-1-(3-bromo-1-((S)-2-((tert-butyldimethylsilyl)oxy)propyl)-1H-pyrazol-5-yl)ethylidene)-2-methylpropane-2-sulfinamide (1.80 g, 3.87 mmol) in THF (30 mL) was added L-selectride (1.0 M in THF, 11.6 mL, 11.6 mmol) dropwise at −78° C. After stirring for 2 h, the reaction was warmed to rt. The reaction was then quenched with NH₄C1 aqueous solution. The mixture was taken up with EtOAc, washed with brine, dried over anhydrous Na₂SO₄, filtered and concentrated. The crude was purified by column chromatography on silica gel eluting with (petroleum ether/EtOAc=50/1-1:1) to give (R)—N—((S)-1-(3-bromo-1-((S)-2-((tert-butyldimethylsilyl)oxy)propyl)-1H-pyrazol-5-yl)ethyl)-2-methylpropane-2-sulfinamide (1.60 g, 89% yield) as a white solid. LCMS ESI (+) m/z 466.1 (M+H).

Step E: Preparation of (R)—N—((S)-1-(3-bromo-1-((S)-2-hydroxypropyl)-1H-pyrazol-5-yl)ethyl)-2-methylpropane-2-sulfinamide: A mixture of (R)—N—((S)-1-(3-bromo-1-((S)-2-((tert-butyldimethylsilyl)oxy)propyl)-1H-pyrazol-5-yl)ethyl)-2-methylpropane-2-sulfinamide (1.00 g, 2.14 mmol) and TBAF (1.68 g, 6.43 mmol) in THF (10 mL) was stirred at 25° C. overnight. The mixture was poured into water and the product was extracted with EtOAc. The combined organic layers were washed with brine, dried over Na₂SO₄, filtered and concentrated. The residue was purified by column chromatography on silica gel eluting with (petroleum ether:EtOAc 3:1→0:1) to afford (R)—N—((S)-1-(3-bromo-1-((S)-2-hydroxypropyl)-1H-pyrazol-5-yl)ethyl)-2-methylpropane-2-sulfinamide (620 mg, 82% yield) as a white solid. LCMS ESI (+) m/z 352.0 (M+H).

Step F: Preparation of (S)-1-(3-bromo-5-((S)-1-(((R)-tert-butylsulfinyl)amino)ethyl)-1H-pyrazol-1-yl)propan-2-yl methanesulfonate: To a solution of (R)—N—((S)-1-(3-bromo-1-((S)-2-hydroxypropyl)-1H-pyrazol-5-yl)ethyl)-2-methylpropane-2-sulfinamide (620 mg, 1.76 mmol) and TEA (1070 mg, 10.6 mmol) in DCM (20 mL) was added methanesulfonyl chloride (0.21 mL, 2.6 mmol) in DCM (2 mL) dropwise at 0° C. After stirring for 30 min, the reaction was warmed to rt and stirred overnight. The reaction was quenched with NaHCO₃ aqueous solution and the mixture was extracted with DCM. The organic phase was washed with brine, dried over anhydrous Na₂SO₄, filtered and concentrated to afford crude (S)-1-(3-bromo-5-((S)-1-(((R)-tert-butylsulfinyl)amino)ethyl)-1H-pyrazol-1-yl)propan-2-yl methanesulfonate (740 mg, 98% yield) as a light brown solid. LCMS ESI (+) m/z 430.0 (M+H).

Step G: Preparation of (S)-1-(5-((S)-1-aminoethyl)-3-bromo-1H-pyrazol-1-yl)propan-2-yl methanesulfonate: To a mixture of (S)-1-(3-bromo-5-((S)-1-(((R)-tert-butylsulfinyl)amino)ethyl)-1H-pyrazol-1-yl)propan-2-yl methanesulfonate (320 mg, 0.744 mmol) in methanol (2 mL) was added 4 N HCl in dioxane (1.0 mL) at 0° C. The mixture was stirred at 25° C. for 2 h. The solvent was removed under vacuum to afford (S)-1-(5-((S)-1-aminoethyl)-3-bromo-1H-pyrazol-1-yl)propan-2-yl methanesulfonate (212 mg, 87% yield) as a brown solid. LCMS ESI (+) m/z 326.0 (M+H).

Step H: Preparation of (4S,6R)-2-bromo-4,6-dimethyl-4,5,6,7-tetrahydropyrazolo[1,5-a]pyrazine: A mixture of (S)-1-(5-((S)-1-aminoethyl)-3-bromo-1H-pyrazol-1-yl)propan-2-yl methanesulfonate (212 mg, 0.650 mmol) and K₂CO₃ (269 mg, 1.95 mmol) in isopropanol (5 mL) and water (2 mL) was stirred at 80° C. for 3 hours. The mixture was diluted with EtOAc, washed with brine, dried over Na₂SO₄, filtered and concentrated to dryness to afford crude (4S,6R)-2-bromo-4,6-dimethyl-4,5,6,7-tetrahydropyrazolo[1,5-a]pyrazine (164 mg) which was used in the next step without further purification. LCMS ESI (+) m/z 230.0 (M+H).

Step I: Preparation of tert-butyl (4S,6R)-2-bromo-4,6-dimethyl-6,7-dihydropyrazolo[1,5-a]pyrazine-5(4H)-carboxylate: A mixture of (4S,6R)-2-bromo-4,6-dimethyl-4,5,6,7-tetrahydropyrazolo[1,5-a]pyrazine (373 mg, 0.811 mmol), di-tert-butyl dicarbonate (0.560 mL, 2.43 mmol) and K₂CO₃ (336 mg, 2.43 mmol) in isopropanol (5 mL) and water (1 mL) was stirred at 25° C. overnight. The reaction mixture was taken up with EtOAc, washed with brine, dried and concentrated. The residue was purified by preparative TLC (petroleum ether/EtOAc=4/1) to afford tert-butyl (4S,6R)-2-bromo-4,6-dimethyl-6,7-dihydro-4H-pyrazolo[1,5-a]pyrazine-5-carboxylate (110 mg, 41% yield). LCMS ESI (+) m/z 330.0 (M+H).

Step J: Preparation of tert-butyl (4S,6R)-2-cyano-4,6-dimethyl-6,7-dihydropyrazolo[1,5-a]pyrazine-5(4H)-carboxylate: A mixture of tert-butyl (4S,6R)-2-bromo-4,6-dimethyl-6,7-dihydro-4H-pyrazolo[1,5-a]pyrazine-5-carboxylate (110 mg, 0.333 mmol), zinc cyanide (39 mg, 0.33 mmol), Zn power (5.4 mg, 0.083 mmol), Pd₂(dba)₃ (46 mg, 0.050 mmol) and dppf (37 mg, 0.0067 mmol) in DMA (3 mL) was stirred at 120° C. for 16 h under Ar. After cooling, the reaction was taken up in EtOAc, washed with brine, dried over Na₂SO₄ and concentrated. The crude was purified by preparative TLC (petroleum ether/EtOAc=3/1) to afford tert-butyl (4S,6R)-2-cyano-4,6-dimethyl-6,7-dihydro-4H-pyrazolo[1,5-a]pyrazine-5-carboxylate (52 mg, 56% yield). LCMS ESI (+) m/z 277.1 (M+H).

Step K: Preparation of tert-butyl (4S,6R)-2-(7-(2,4-difluoro-6-(2-methoxyethoxy)phenyl)-4-hydroxythieno[3,2-c]pyridin-6-yl)-4,6-dimethyl-6,7-dihydropyrazolo[1,5-a]pyrazine-5(4H)-carboxylate: To a solution of 2-[[2,4-difluoro-6-(2-methoxyethoxy)phenyl]methyl]-N,N-diethyl-thiophene-3-carboxamide (48 mg, 0.12 mmol) in THF (2 mL) was added n-butyllithium (0.05 mL, 2.5 M in hexanes, 0.13 mmol) at −78° C. dropwise. After stirring for 30 min, tert-butyl (4S,6R)-2-cyano-4,6-dimethyl-6,7-dihydro-4H-pyrazolo[1,5-a]pyrazine-5-carboxylate (34 mg, 0.12 mmol) in THF (0.5 mL) was added at −78° C. The reaction was warmed to rt and stirred for additional 3 h. After quenched with NH₄Cl aqueous solution, the mixture was extracted with EtOAc and washed with brine. The organic phase was dried over Na₂SO₄, filtered and concentrated. The residue was purified by preparative TLC (petroleum ether/EtOAc=1/1) to afford crude tert-butyl (4S,6R)-2-[7-[2,4-difluoro-6-(2-methoxyethoxy)phenyl]-4-hydroxy-thieno[3,2-c]pyridin-6-yl]-4,6-dimethyl-6,7-dihydro-4H-pyrazolo[1,5-a]pyrazine-5-carboxylate (66 mg, 93% yield). LCMS ESI (+) m/z 587.0 (M+H).

Step L: Preparation of tert-butyl (4S,6R)-2-(7-(2,4-difluoro-6-(2-methoxyethoxy)phenyl)-4-(((trifluoromethyl)sulfonyl)oxy)thieno[3,2-c]pyridin-6-yl)-4,6-dimethyl-6,7-dihydropyrazolo[1,5-a]pyrazine-5(4H)-carboxylate: To a mixture of tert-butyl (4S,6R)-2-[7-[2,4-difluoro-6-(2-methoxyethoxy)phenyl]-4-hydroxy-thieno[3,2-c]pyridin-6-yl]-4,6-dimethyl-6,7-dihydro-4H-pyrazolo[1,5-a]pyrazine-5-carboxylate (66 mg, 0.11 mmol) and TEA (114 mg, 1.13 mmol) in DCM (2 mL) was added trifluoromethanesulfonic anhydride (0.038 mL, 0.23 mmol) at 0° C. The mixture was stirred at 0° C. for another 2 h. The reaction was taken up with DCM, washed with NaHCO₃ aqueous solution and brine solution. The organic phase was dried over Na₂SO₄, filtered and concentrated. The residue was purified by preparative TLC (petroleum ether/EtOAc=3/1) to afford tert-butyl (4S,6R)-2-(7-(2,4-difluoro-6-(2-methoxyethoxy)phenyl)-4-(((trifluoromethyl)sulfonyl)oxy)thieno[3,2-c]pyridin-6-yl)-4,6-dimethyl-6,7-dihydropyrazolo[1,5-a]pyrazine-5(4H)-carboxylate (71 mg, 88% yield).

Step M: Preparation of 7-(2,4-difluoro-6-(2-methoxyethoxy)phenyl)-6-((4S,6R)-4,6-dimethyl-4,5,6,7-tetrahydropyrazolo[1,5-a]pyrazin-2-yl)thieno[3,2-c]pyridin-4-yl trifluoromethanesulfonate: A mixture of tert-butyl (4S,6R)-2-(7-(2,4-difluoro-6-(2-methoxyethoxy)phenyl)-4-(((trifluoromethyl)sulfonyl)oxy)thieno[3,2-c]pyridin-6-yl)-4,6-dimethyl-6,7-dihydropyrazolo[1,5-a]pyrazine-5(4H)-carboxylate (71 mg, 0.099 mmol) in DCM (2 mL) was added trifluoroacetic acid (0.15 mL). The mixture was stirred at 20° C. for 2 h. The solvent was removed under vacuum to afford crude 7-(2,4-difluoro-6-(2-methoxyethoxy)phenyl)-6-((4S,6R)-4,6-dimethyl-4,5,6,7-tetrahydropyrazolo[1,5-a]pyrazin-2-yl)thieno[3,2-c]pyridin-4-yl trifluoromethanesulfonate which was used directly in the next step.

Step N: Preparation of 6-((4S,6R)-5-acryloyl-4,6-dimethyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate: The crude product from Step M was dissolved in DCM (3 mL). TEA (100 mg, 0.988 mmol) was added followed by dropwise addition of acryloyl chloride (0.016 mL, 0.20 mmol) in DCM (0.5 mL) at −20° C. The reaction was stirred at −20° C. for 3 h. The mixture was poured into NaHCO₃ aqueous solution, and the mixture was extracted with EtOAc. The organic phase was washed with brine solution, dried over Na₂SO₄, filtered and concentrated. The residue was purified by preparative TLC (petroleum ether/EtOAc=3/1) to afford 6-((4S,6R)-5-acryloyl-4,6-dimethyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate (34 mg, 51% yield). LCMS ESI (+) m/z 672.9 (M+H).

Step O: Preparation of 1-((4S,6R)-2-(7-(2,4-difluoro-6-(2-methoxyethoxy)phenyl)-4-(2-methyl-1,2,3,4-tetrahydroisoquinolin-6-yl)thieno[3,2-c]pyridin-6-yl)-4,6-dimethyl-6,7-dihydropyrazolo[1,5-a]pyrazin-5(4H)-yl)prop-2-en-1-one: A mixture of 6-((4S,6R)-5-acryloyl-4,6-dimethyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate (34 mg, 0.051 mmol), Pd(PPh₃)₄(3.7 mg, 0.0051 mmol) and Na₂CO₃ (11 mg, 0.10 mmol) in 1,4-dioxane (5 mL) and water (1 mL) was stirred at 90° C. for 4 h under Ar. After cooling to rt, the mixture was concentrated and purified by preparative TLC (DCM/methanol=20/1) and preparative HPLC to afford 1-[(4S,6R)-2-[7-[2,4-difluoro-6-(2-methoxyethoxy)phenyl]-4-(2-methyl-3,4-dihydro-1H-isoquinolin-6-yl)thieno[3,2-c]pyridin-6-yl]-4,6-dimethyl-6,7-dihydro-4H-pyrazolo[1,5-a]pyrazin-5-yl]prop-2-en-1-one (17 mg, 50% yield) as a bis trifluoroacetic acid salt. ¹H NMR (400 MHz, CD₃OD) δ 7.84-7.95 (m, 2H), 7.78 (d, J=5.5 Hz, 1H), 7.66 (d, J=5.1 Hz, 1H), 7.44 (d, J=8.1 Hz, 1H), 6.79-6.90 (m, 2H), 6.63-6.73 (m, 1H), 6.25-6.37 (m, 2H), 5.81 (d, J=10.4 Hz, 1H), 5.30-5.42 (m, 1H), 4.63-4.74 (m, 2H), 4.41-4.60 (m, 2H), 3.71-4.21 (m, 4H), 3.33-3.50 (m, 5H), 3.13 (s, 3H), 3.05 (s, 3H), 1.38-1.49 (m, 3H), 0.81-0.92 (m, 3H). LCMS ESI (+) m/z 670.0 (M+H).

### Synthetic Example 61: Synthesis of 1-((S)-2-((R)-7-(2,4-difluoro-6-((R)-2-hydroxypropoxy)phenyl)-4-(2-methyl-2H-indazol-5-yl)thieno[3,2-c]pyridin-6-yl)-7-methyl-6,7-dihydropyrazolo[1,5-a]pyrazin-5(4H)-yl)prop-2-en-1-one (Compound 571) and 1-((S)-2-((S)-7-(2,4-difluoro-6-((R)-2-hydroxypropoxy)phenyl)-4-(2-methyl-2H-indazol-5-yl)thieno[3,2-c]pyridin-6-yl)-7-methyl-6,7-dihydropyrazolo[1,5-a]pyrazin-5(4H)-yl)prop-2-en-1-one (Compound 572)

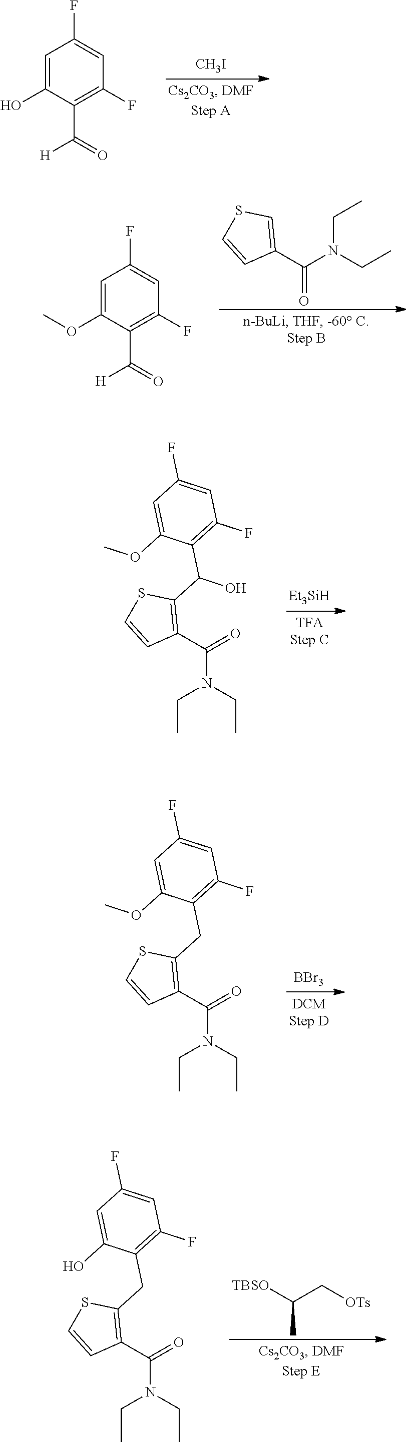

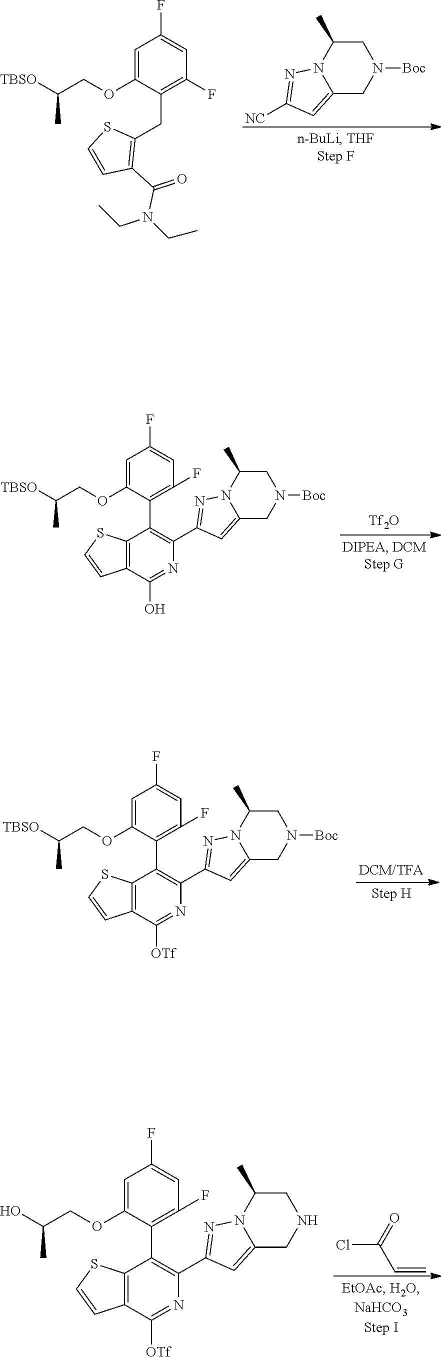

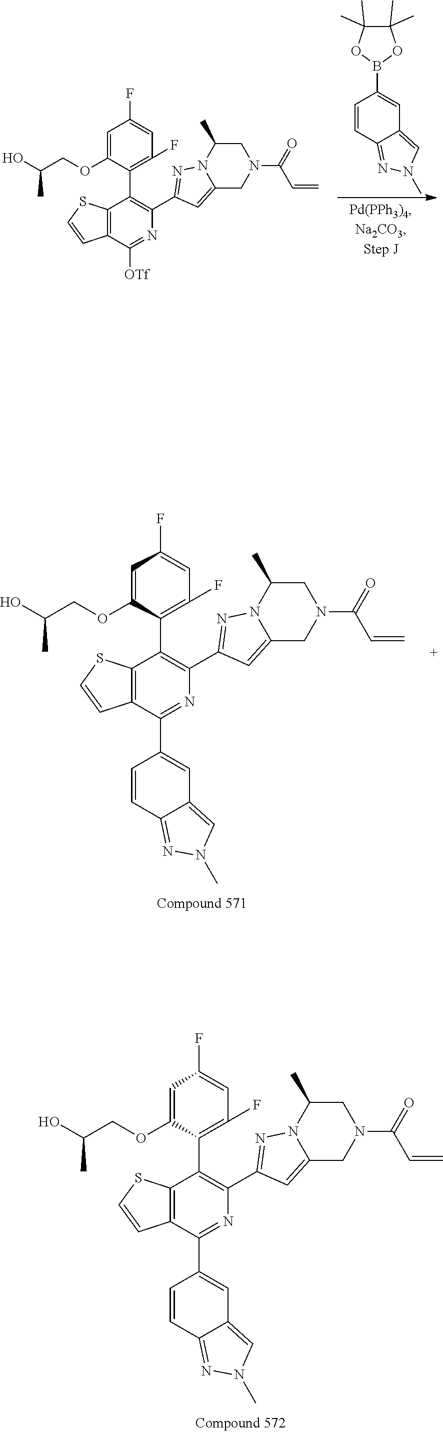

Step A: Preparation of 2,4-difluoro-6-methoxy-benzaldehyde: A mixture of 2,4-difluoro-6-hydroxy-benzaldehyde (10.0 g, 63.3 mmol), potassium carbonate (17.0 g, 123 mmol) and methyl iodide (5.70 mL, 91.6 mmol) in DMF (150 mL) was stirred at 50° C. in a sealed tube for 3 h. The mixture was poured into water (200 mL) and extracted with MTBE. The organic phase was washed with brine solution, dried over Na₂SO₄, filtered and concentrated to dryness to give 2,4-difluoro-6-methoxy-benzaldehyde (10.0 g, 92% yield) as a light-yellow oil.

Step B: Preparation of 2-[(2,4-difluoro-6-methoxy-phenyl)-hydroxy-methyl]-N,N-diethyl-thiophene-3-carboxamide: To a solution of N,N-diethylthiophene-3-carboxamide (20.4 g, 111 mmol) in THF (5 mL) was added n-butyllithium (2.5 M in hexanes, 48.0 mL, 121 mmol) dropwise under Ar at −78° C. The mixture was stirred at −60° C. for 0.5 h, and then 2,4-difluoro-6-methoxy-benzaldehyde (17.4 g, 101 mmol) was added at −60° C. The temperature was allowed to warm to 0° C. The reaction was stirred at 0° C. for additional 1 h. The mixture was poured into water. Aqueous 1 N HCl solution was added dropwise to adjust the pH to 5. The mixture was extracted with EtOAc, dried over anhydrous Na₂SO₄, concentrated and purified by silica gel column chromatography eluting with EtOAc/petroleum ether (1/1) to give 2-[(2,4-difluoro-6-methoxy-phenyl)-hydroxy-methyl]-N,N-diethyl-thiophene-3-carboxamide (28.0 g, 78% yield). LCMS ESI (+) m/z 338.1 (M+1-H₂O).

Step C: Preparation of 2-[(2,4-difluoro-6-methoxy-phenyl)methyl]-N,N-diethyl-thiophene-3-carboxamide: To a solution of 2-[(2,4-difluoro-6-methoxy-phenyl)-hydroxy-methyl]-N,N-diethyl-thiophene-3-carboxamide (3.60 g, 10.1 mmol) in trifluoroacetic acid (72 mL) was added triethylsilane (11.0 mL, 70.9 mmol) under Ar. The mixture was stirred at rt for 2 hours. The mixture was concentrated and the residue was dissolved in EtOAc. The organics were washed with saturated aqueous NaHCO₃, water and brine, dried and concentrated. The crude was purified by flash column chromatography on silica gel eluting with 25% EtOAc in petroleum ether to give 2-[(2,4-difluoro-6-methoxy-phenyl)methyl]-N,N-diethyl-thiophene-3-carboxamide (3.00 g, 87% yield).

Step D: Preparation of 2-[(2,4-difluoro-6-hydroxy-phenyl)methyl]-N,N-diethyl-thiophene-3-carboxamide: To a solution of 2-[(2,4-difluoro-6-methoxy-phenyl)methyl]-N,N-diethyl-thiophene-3-carboxamide (19.5 g, 57.5 mmol) in DCM (150 mL) was added boron tribromide (72.0 g, 287 mmol) at −40° C. The mixture was stirred at 0° C. for 1.5 h. The mixture was poured into water and aqueous Na₂SO₃ solution was added. The mixture was extracted with EtOAc, washed with brine, dried over Na₂SO₄, filtered and concentrated to dryness to give 2-[(2,4-difluoro-6-hydroxy-phenyl)methyl]-N,N-diethyl-thiophene-3-carboxamide (17.0 g, 91% yield) as an off-white solid. LCMS ESI (+) m/z 326 (M+H).

Step E: Preparation of 2-[[2-[(2R)-2-[tert-butyl(dimethyl)silyl]oxypropoxy]-4,6-difluoro-phenyl]methyl]-N,N-diethyl-thiophene-3-carboxamide: To a solution of 2-[(2,4-difluoro-6-hydroxy-phenyl)methyl]-N,N-diethyl-thiophene-3-carboxamide (150 mg, 0.461 mmol) and Cs₂CO₃ (225 mg, 0.692 mmol) in DMF (5 mL) was added [(2R)-2-[tert-butyl(dimethyl)silyl]oxypropyl]4-methylbenzenesulfonate (159 mg, 0.461 mmol). The mixture was stirred at 70° C. for 5 h. The mixture was poured into water and the product was extracted with EtOAc. The organics were washed with brine, dried over anhydrous Na₂SO₄, concentrated and purified by preparative TLC (EtOAc/petroleum ether: 1/4) to give 2-[[2-[(2R)-2-[tert-butyl(dimethyl)silyl]oxypropoxy]-4,6-difluoro-phenyl]methyl]-N,N-diethyl-thiophene-3-carboxamide (160 mg, 70% yield). ¹H NMR (400 MHz, CDCl₃) δ 6.95 (d, J=5.2 Hz, 1H), 6.77 (d, J=5.2 Hz, 1H), 6.28-6.41 (m, 2H), 4.05-4.13 (m, 3H), 3.82-3.87 (m, 1H), 3.64-3.69 (m, 1H), 3.40-3.52 (m, 2H), 3.08-3.20 (m, 2H), 1.10-1.21 (m, 6H). 0.98-1.10 (m, 3H), 0.80 (s, 9H), 0.00 (s, 3H), −0.03 (s, 3H).

Step F: Preparation of tert-butyl (7S)-2-[7-[2-[(2R)-2-[tert-butyl(dimethyl)silyl]oxypropoxy]-4,6-difluoro-phenyl]-4-hydroxy-thieno[3,2-c]pyridin-6-yl]-7-methyl-6,7-dihydro-4H-pyrazolo[1,5-a]pyrazine-5-carboxylate: To a solution of 2-[[2-[(2R)-2-[tert-butyl(dimethyl)silyl]oxypropoxy]-4,6-difluoro-phenyl]methyl]-N,N-diethyl-thiophene-3-carboxamide (152 mg, 0.306 mmol) in THF (3 mL) was added n-BuLi (2.5 M in hexanes, 0.134 mL, 0.334 mmol) dropwise under Ar at −78° C. The mixture was stirred at −60° C. for 0.5 h under Ar. A solution of tert-butyl (7S)-2-cyano-7-methyl-6,7-dihydro-4H-pyrazolo[1,5-a]pyrazine-5-carboxylate (73 mg, 0.28 mmol) in THF (0.5 mL) was added at −60° C. The temperature was allowed to warm to 0° C., and the resulting mixture was stirred at 0° C. for additional 1 h. The mixture was poured into water and 1 N aqueous HCl was added to adjust the pH to 5. The mixture was diluted with water. The product was extracted with EtOAc, washed with saturated brine solution, dried over Na₂SO₄, filtered and concentrated. The residue was purified by preparative TLC (EtOAc/petroleum ether: 1/2) to give tert-butyl (7S)-2-[7-[2-[(2R)-2-[tert-butyl(dimethyl)silyl]oxypropoxy]-4,6-difluoro-phenyl]-4-hydroxy-thieno[3,2-c]pyridin-6-yl]-7-methyl-6,7-dihydro-4H-pyrazolo[1,5-a]pyrazine-5-carboxylate (120 mg, 63% yield).

Step G: Preparation of tert-butyl (7S)-2-[7-[2-[(2R)-2-[tert-butyl(dimethyl)silyl]oxypropoxy]-4,6-difluoro-phenyl]-4-(trifluoromethylsulfonyloxy)thieno[3,2-c]pyridin-6-yl]-7-methyl-6,7-dihydro-4H-pyrazolo[1,5-a]pyrazine-5-carboxylate: To a solution of tert-butyl (7S)-2-[7-[2-[(2R)-2-[tert-butyl(dimethyl)silyl]oxypropoxy]-4,6-difluoro-phenyl]-4-hydroxy-thieno[3,2-c]pyridin-6-yl]-7-methyl-6,7-dihydro-4H-pyrazolo[1,5-a]pyrazine-5-carboxylate (120 mg, 0.175 mmol) and pyridine (0.0420 mL, 0.524 mmol) in DCM (5 mL) was added trifluoromethanesulfonic anhydride (0.059 mL, 0.35 mmol). The mixture was stirred at 25° C. for 1 h. The mixture was poured into water and the product was extracted with DCM. The organics were washed with brine, dried over anhydrous Na₂SO₄, concentrated and purified by preparative TLC (EtOAc/petroleum ether: 1/6) to give tert-butyl (7S)-2-[7-[2-[(2R)-2-[tert-butyl(dimethyl)silyl]oxypropoxy]-4,6-difluoro-phenyl]-4-(trifluoromethylsulfonyloxy)thieno[3,2-c]pyridin-6-yl]-7-methyl-6,7-dihydro-4H-pyrazolo[1,5-a]pyrazine-5-carboxylate (125 mg, 87% yield).

Step H: Preparation of [7-[2,4-difluoro-6-[(2R)-2-hydroxypropoxy]phenyl]-6-[(7S)-7-methyl-4,5,6,7-tetrahydropyrazolo[1,5-a]pyrazin-2-yl]thieno[3,2-c]pyridin-4-yl]trifluoromethanesulfonate: tert-butyl (7S)-2-[7-[2-[(2R)-2-[tert-butyl(dimethyl)silyl]oxypropoxy]-4,6-difluoro-phenyl]-4-(trifluoromethylsulfonyloxy)thieno[3,2-c]pyridin-6-yl]-7-methyl-6,7-dihydro-4H-pyrazolo[1,5-a]pyrazine-5-carboxylate (125 mg, 0.153 mmol) was dissolved in DCM (3 mL) and trifluoroacetic acid (1.0 mL) was added. The mixture was stirred for 1 h. The solvent was concentrated under reduced pressure to afford crude [7-[2,4-difluoro-6-[(2R)-2-hydroxypropoxy]phenyl]-6-[(7S)-7-methyl-4,5,6,7-tetrahydropyrazolo[1,5-a]pyrazin-2-yl]thieno[3,2-c]pyridin-4-yl]trifluoromethanesulfonate (135 mg), which was used in the next step without further purification.

Step I: Preparation of 7-[2,4-difluoro-6-[(2R)-2-hydroxypropoxy]phenyl]-6-[(7S)-7-methyl-5-prop-2-enoyl-6,7-dihydro-4H-pyrazolo[1,5-a]pyrazin-2-yl]thieno[3,2-c]pyridin-4-yl]trifluoromethanesulfonate: To a solution of [7-[2,4-difluoro-6-[(2R)-2-hydroxypropoxy]phenyl]-6-[(7S)-7-methyl-4,5,6,7-tetrahydropyrazolo[1,5-a]pyrazin-2-yl]thieno[3,2-c]pyridin-4-yl]trifluoromethanesulfonate (135 mg, 0.125 mmol) in DCM (5 mL) was added N,N-diisopropylethylamine (0.076 mL, 0.44 mmol), followed by acryloyl chloride (12 mg, 0.14 mmol) at −20° C. The mixture was stirred at −20° C. for 30 mins. The mixture was poured into NaHCO₃ aqueous solution and the product was extracted with EtOAc. The combined organics were washed with brine, dried over anhydrous Na₂SO₄ and concentrated to give [7-[2,4-difluoro-6-[(2R)-2-hydroxypropoxy]phenyl]-6-[(7S)-7-methyl-5-prop-2-enoyl-6,7-dihydro-4H-pyrazolo[1,5-a]pyrazin-2-yl]thieno[3,2-c]pyridin-4-yl]trifluoromethanesulfonate (120 mg, 100% yield).

Step J: Preparation of 1-((S)-2-((R)-7-(2,4-difluoro-6-((R)-2-hydroxypropoxy)phenyl)-4-(2-methyl-2H-indazol-5-yl)thieno[3,2-c]pyridin-6-yl)-7-methyl-6,7-dihydropyrazolo[1,5-a]pyrazin-5(4H)-yl)prop-2-en-1-one (Compound 571) and 1-((S)-2-((S)-7-(2,4-difluoro-6-((R)-2-hydroxypropoxy)phenyl)-4-(2-methyl-2H-indazol-5-yl)thieno[3,2-c]pyridin-6-yl)-7-methyl-6,7-dihydropyrazolo[1,5-a]pyrazin-5(4H)-yl)prop-2-en-1-one (Compound 572): To a solution of [7-[2,4-difluoro-6-[(2R)-2-hydroxypropoxy]phenyl]-6-[(7S)-7-methyl-5-prop-2-enoyl-6,7-dihydro-4H-pyrazolo[1,5-a]pyrazin-2-yl]thieno[3,2-c]pyridin-4-yl]trifluoromethanesulfonate (40 mg, 0.061 mmol), 2-methyl-5-(4,4,5,5-tetramethyl-1,3,2-dioxaborolan-2-yl)indazole (20 mg, 0.079 mmol) and Na₂CO₃ (19 mg, 0.18 mmol) in 1,4-dioxane (2 mL) and water (0.2 mL) was added Pd(PPh₃)₄(9.1 mg, 0.0079 mmol). The mixture was stirred at 100° C. for 4 h under Ar. The mixture was poured into water and the product was extracted with EtOAc. The combined organics were washed with brine, dried over anhydrous Na₂SO₄, concentrated and purified by preparative HPLC to give 1-((S)-2-((R)-7-(2,4-difluoro-6-((R)-2-hydroxypropoxy)phenyl)-4-(2-methyl-2H-indazol-5-yl)thieno[3,2-c]pyridin-6-yl)-7-methyl-6,7-dihydropyrazolo[1,5-a]pyrazin-5(4H)-yl)prop-2-en-1-one (Compound 571) (7.1 mg, 18% yield) and 1-((S)-2-((S)-7-(2,4-difluoro-6-((R)-2-hydroxypropoxy)phenyl)-4-(2-methyl-2H-indazol-5-yl)thieno[3,2-c]pyridin-6-yl)-7-methyl-6,7-dihydropyrazolo[1,5-a]pyrazin-5(4H)-yl)prop-2-en-1-one (Compound 572) (5.5 mg, 14% yield). Compound 571: ¹H NMR (400 MHz, CD₃OD) δ 8.25 (s, 1H), 8.18 (s, 1H), 7.83 (dd, J=9.0, 1.4 Hz, 1H), 7.68 (d, J=9.0 Hz, 1H), 7.60 (s, 2H), 6.55-6.83 (m, 3H), 6.32 (s, 1H), 6.18 (d, J=16.6 Hz, 1H), 5.71 (d, J=10.8 Hz, 1H), 4.17 (s, 4H), 3.91-4.05 (m, 1H), 3.68-3.82 (m, 2H), 3.48-3.65 (m, 2H), 1.17-1.36 (m, 3H), 0.74 (d, J=6.3 Hz, 3H); LCMS ESI (+) m/z 641.0 (M+H). Compound 572: ¹H NMR (400 MHz, CD₃OD) δ 8.37 (s, 1H), 8.30 (s, 1H), 7.94 (d, J=8.8 Hz, 1H), 7.79 (d, J=8.8 Hz, 1H), 7.71 (s, 2H), 6.78-6.95 (m, 2H), 6.58-6.68 (m, 1H), 6.45 (d, J=9.6 Hz, 1H), 6.29 (d, J=16.5 Hz, 1H), 5.82 (d, J=10.6 Hz, 1H), 4.28 (s, 4H), 4.07-4.16 (m, 1H), 3.71-3.94 (m, 4H), 1.21-1.39 (m, 3H), 0.90 (d, J=6.2 Hz, 3H); LCMS ESI (+) m/z 641.0 (M+H).

### Synthetic Example 62: Synthesis of 1-(2-(5-fluoro-4-(4-fluoro-2-methoxyphenyl)-1-(1-methyl-1H-pyrazol-4-yl)-6,7-dihydro-5H-cyclopenta[c]pyridin-3-yl)-6,7-dihydropyrazolo[1,5-a]pyrazin-5(4H)-yl)prop-2-en-1-one (Compound 574)

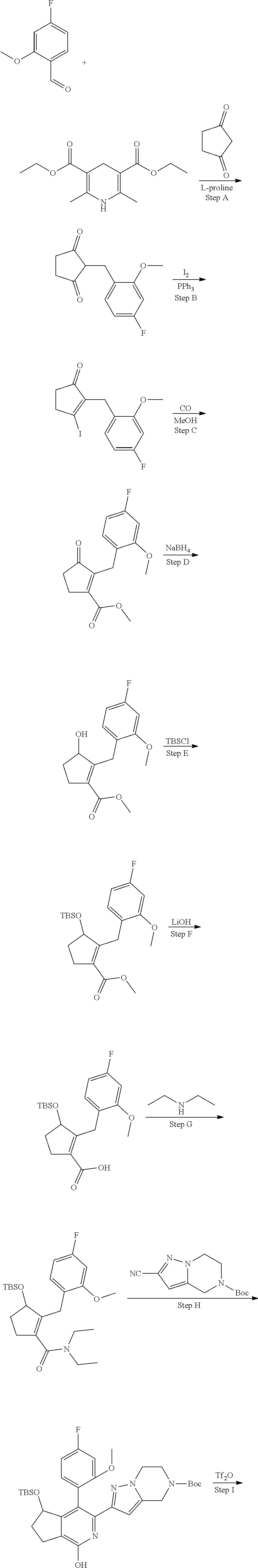

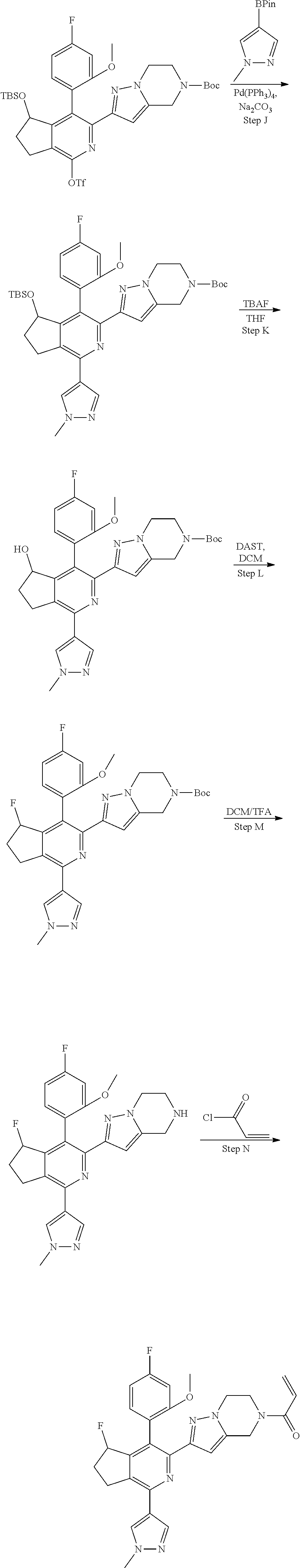

Step A: Preparation of 2-[(4-fluoro-2-methoxy-phenyl)methyl]cyclopentane-1,3-dione: A mixture of cyclopentane-1,3-dione (2.50 g, 25.5 mmol), 4-fluoro-2-methoxy-benzaldehyde (11.8 g, 76.5 mmol), diethyl 2,6-dimethyl-1,4-dihydropyridine-3,5-dicarboxylate (6.45 g, 25.5 mmol) and L-proline (147 mg, 1.27 mmol) in DCM (85 mL) was stirred at rt overnight. The solvent was removed by rotatory evaporator. The residue was purified by column chromatography on silica gel (petroleum ether/EtOAc=3/1 to EtOAc) to give 2-[(4-fluoro-2-methoxy-phenyl)methyl]cyclopentane-1,3-dione (5.00 g, 83% yield). LCMS ESI (+) m/z 237.1 (M+H).

Step B: Preparation of 2-[(4-fluoro-2-methoxy-phenyl)methyl]-3-iodo-cyclopent-2-en-1-one: To a solution of triphenylphosphine (6.65 g, 25.4 mmol) in acetonitrile (120 mL) was added iodine (6.44 g, 25.4 mmol). The mixture was stirred at rt for 0.5 h. 2-[(4-fluoro-2-methoxy-phenyl)methyl]cyclopentane-1,3-dione (4.61 g, 19.5 mmol) was added and stirred at 82° C. overnight. Saturated sodium thiosulfate solution was added to quench the reaction. The mixture was extracted with EtOAc, dried (Na₂SO₄), filtered and concentrated. The residue was purified by column chromatography on silica gel (petroleum ether/EtOAc=10/1) to give crude 2-[(4-fluoro-2-methoxy-phenyl)methyl]-3-iodo-cyclopent-2-en-1-one (7.14 g) which was used in the next step directly. LCMS ESI (+) m/z 347.0 (M+H).

Step C: Preparation of methyl 2-[(4-fluoro-2-methoxy-phenyl)methyl]-3-oxo-cyclopentene-1-carboxylate: A mixture of 2-[(4-fluoro-2-methoxy-phenyl)methyl]-3-iodo-cyclopent-2-en-1-one (3.00 g, 8.67 mmol), Pd(dppf)Cl₂ (634 mg, 0.867 mmol) and TEA (4.39 g, 43.3 mmol) in methanol (35 mL) was stirred at 100° C. for 5 h under CO. The solvent was removed by rotatory evaporator, and the residue was purified by column chromatography on silica gel (petroleum ether/EtOAc=3/1) to give methyl 2-[(4-fluoro-2-methoxy-phenyl)methyl]-3-oxo-cyclopentene-1-carboxylate (2.06 g, 85% yield). LCMS ESI (+) m/z 279.1 (M+H).

Step D: Preparation of methyl 2-[(4-fluoro-2-methoxy-phenyl)methyl]-3-hydroxy-cyclopentene-1-carboxylate: To a solution of methyl 2-[(4-fluoro-2-methoxy-phenyl)methyl]-3-oxo-cyclopentene-1-carboxylate (1.00 g, 3.59 mmol) in methanol (25 mL) was added sodium borohydride (272 mg, 7.19 mmol) in portions at 0° C. The mixture was stirred at 15° C. for 1 h. The reaction was quenched with water and extracted with DCM twice. The organic phases were washed with brine, dried (Na₂SO₄) and concentrated to afford methyl 2-[(4-fluoro-2-methoxy-phenyl)methyl]-3-hydroxy-cyclopentene-1-carboxylate (1.00 g, 99% yield), which was used in next step directly. ¹H NMR (400 MHz, CDCl₃) δ 7.20-7.26 (m, 1H), 6.56-6.65 (m, 2H), 4.51-4.60 (m, 1H), 4.25-4.36 (m, 1H), 3.87 (s, 3H), 3.82 (s, 3H), 3.60-3.72 (m, 1H), 2.70-2.81 (m, 2H), 2.35-2.50 (m, 1H), 2.12-2.27 (m, 1H), 1.60-1.75 (m, 1H).

Step E: Preparation of methyl 3-[tert-butyl(dimethyl)silyl]oxy-2-[(4-fluoro-2-methoxy-phenyl)methyl]cyclopentene-1-carboxylate: To a solution of methyl 2-[(4-fluoro-2-methoxy-phenyl)methyl]-3-hydroxy-cyclopentene-1-carboxylate (1.00 g, 3.57 mmol) in DCM (15 mL) was added imidazole (486 mg, 7.14 mmol), followed by tert-butyldimethylchlorosilane (645 mg, 4.28 mmol). The reaction was stirred at 20° C. for 2 h. After concentration, the reaction was diluted with EtOAc and washed with brine. The organic phase was dried and concentrated. The crude was purified by column chromatography on silica gel (petroleum ether/EtOAc=10/1) to afford methyl 3-[tert-butyl(dimethyl)silyl]oxy-2-[(4-fluoro-2-methoxy-phenyl)methyl]cyclopentene-1-carboxylate (1.40 g, 99% yield). ¹H NMR (400 MHz, CDCl₃) δ 7.02-7.08 (m, 1H), 6.64-6.69 (m, 2H), 4.75-4.82 (m, 1H), 4.11-4.20 (m, 1H), 3.91 (s, 3H), 3.81-3.90 (m, 4H), 2.80-2.91 (m, 2H), 2.56-2.70 (m, 1H), 2.25-2.38 (m, 1H), 0.97 (s, 9H), 0.06 (s, 3H), 0.00 (s, 3H).

Step F: Preparation of 3-[tert-butyl(dimethyl)silyl]oxy-2-[(4-fluoro-2-methoxy-phenyl)methyl]cyclopentene-1-carboxylic acid: To a solution of methyl 3-[tert-butyl(dimethyl)silyl]oxy-2-[(4-fluoro-2-methoxy-phenyl)methyl]cyclopentene-1-carboxylate (1.40 g, 3.55 mmol) in THF (15 mL)/methanol (5 mL)/water (5 mL) was added LiOH (1.49 g, 35.5 mmol). The reaction was stirred at 15° C. for 24 h. After concentration, the reaction was diluted with water. 2 N aqueous HCl was added to adjust the pH to 3. The mixture was extracted with EtOAc, dried and concentrated to afford 3-[tert-butyl(dimethyl)silyl]oxy-2-[(4-fluoro-2-methoxy-phenyl)methyl]cyclopentene-1-carboxylic acid (1.30 g, 96% yield). LCMS ESI (−) m/z 379.2 (M−H).

Step G: Preparation of 3-[tert-butyl(dimethyl)silyl]oxy-N,N-diethyl-2-[(4-fluoro-2-methoxy-phenyl)methyl]cyclopentene-1-carboxamide: To a solution of 3-[tert-butyl(dimethyl)silyl]oxy-2-[(4-fluoro-2-methoxy-phenyl)methyl]cyclopentene-1-carboxylic acid (600 mg, 1.58 mmol), triethylamine (0.660 mL, 4.73 mmol) and HATU (779 mg, 2.05 mmol) in DCM (10 mL) was added diethylamine (0.260 mL, 2.52 mmol). The mixture was stirred at 25° C. for 1 h. The mixture was poured into water and the product was extracted with DCM. The organics were washed with brine. The solvent was removed under reduced pressure and the residue was purified by flash chromatography on silica gel (EtOAc/petroleum ether=1/4) to give 3-[tert-butyl(dimethyl)silyl]oxy-N,N-diethyl-2-[(4-fluoro-2-methoxy-phenyl)methyl]cyclopentene-1-carboxamide (710 mg, 103% yield).

Step H: Preparation of tert-butyl 2-[5-[tert-butyl(dimethyl)silyl]oxy-4-(4-fluoro-2-methoxy-phenyl)-1-hydroxy-6,7-dihydro-5H-cyclopenta[c]pyridin-3-yl]-6,7-dihydro-4H-pyrazolo[1,5-a]pyrazine-5-carboxylate: To a solution of 3-[tert-butyl(dimethyl)silyl]oxy-N,N-diethyl-2-[(4-fluoro-2-methoxy-phenyl)methyl]cyclopentene-1-carboxamide (300 mg, 0.689 mmol) in THF (10 mL) was added LDA (2.0 M, 0.500 mL, 1.03 mmol) dropwise at −60° C. under Ar. The mixture was stirred at −60° C. for 0.5 h, and then a solution of tert-butyl 2-cyano-6,7-dihydro-4H-pyrazolo[1,5-a]pyrazine-5-carboxylate (188 mg, 0.757 mmol) in THF (1 mL) was added at −60° C. The temperature was allowed to warm to 0° C., and the resulting mixture was stirred at 0° C. for additional 1 h. The mixture was poured into water and 1 N aqueous HCl was added to adjust the pH to 5. The mixture was extracted with EtOAc, washed with brine solution, dried over Na₂SO₄, filtered and concentrated. The residue was purified by preparative TLC (DCM:MeOH 20/1) to give tert-butyl 2-[5-[tert-butyl(dimethyl)silyl]oxy-4-(4-fluoro-2-methoxy-phenyl)-1-hydroxy-6,7-dihydro-5H-cyclopenta[c]pyridin-3-yl]-6,7-dihydro-4H-pyrazolo[1,5-a]pyrazine-5-carboxylate (156 mg, 37% yield). LCMS ESI (+) m/z 611.3 (M+H).

Step I: Preparation of tert-butyl 2-[5-[tert-butyl(dimethyl)silyl]oxy-4-(4-fluoro-2-methoxy-phenyl)-1-(trifluoromethylsulfonyloxy)-6,7-dihydro-5H-cyclopenta[c]pyridin-3-yl]-6,7-dihydro-4H-pyrazolo[1,5-a]pyrazine-5-carboxylate: To a solution of tert-butyl 2-[5-[tert-butyl(dimethyl)silyl]oxy-4-(4-fluoro-2-methoxy-phenyl)-1-hydroxy-6,7-dihydro-5H-cyclopenta[c]pyridin-3-yl]-6,7-dihydro-4H-pyrazolo[1,5-a]pyrazine-5-carboxylate (210 mg, 0.344 mmol) and pyridine (0.083 mL, 1.0 mmol) in DCM (5 mL) was added trifluoromethanesulfonic anhydride (0.12 mL, 0.69 mmol). The mixture was stirred at 0° C. for 1 h. The mixture was poured into water and the product was extracted with EtOAc. The organics were washed with brine, dried over anhydrous Na₂SO₄, concentrated and purified by preparative TLC (EtOAc/petroleum ether: 1/4) to give tert-butyl 2-[5-[tert-butyl(dimethyl)silyl]oxy-4-(4-fluoro-2-methoxy-phenyl)-1-(trifluoromethylsulfonyloxy)-6,7-dihydro-5H-cyclopenta[c]pyridin-3-yl]-6,7-dihydro-4H-pyrazolo[1,5-a]pyrazine-5-carboxylate (206 mg, 81% yield). LCMS ESI (+) m/z 743.2 (M+H).

Step J: Preparation of tert-butyl 2-[5-[tert-butyl(dimethyl)silyl]oxy-4-(4-fluoro-2-methoxy-phenyl)-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate: To a solution of tert-butyl 2-[5-[tert-butyl(dimethyl)silyl]oxy-4-(4-fluoro-2-methoxy-phenyl)-1-(trifluoromethylsulfonyloxy)-6,7-dihydro-5H-cyclopenta[c]pyridin-3-yl]-6,7-dihydro-4H-pyrazolo[1,5-a]pyrazine-5-carboxylate (176 mg, 0.237 mmol), 1-methyl-4-(4,4,5,5-tetramethyl-1,3,2-dioxaborolan-2-yl)pyrazole (74 mg, 0.36 mmol) and Na₂CO₃ (75 mg, 0.71 mmol) in 1,4-dioxane (3 mL) and water (0.3 mL) was added Pd(PPh₃)₄(26 mg, 0.036 mmol). The mixture was stirred at 100° C. for 6 h under Ar. The mixture was poured into water and the product was extracted with EtOAc. The organics were washed with brine, dried over anhydrous Na₂SO₄, concentrated and purified by preparative TLC (DCM/MeOH=20/1) to give tert-butyl 2-[5-[tert-butyl(dimethyl)silyl]oxy-4-(4-fluoro-2-methoxy-phenyl)-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate (142 mg, 71% yield). LCMS ESI (+) m/z 675.4 (M+H).

Step K: Preparation of tert-butyl 2-[4-(4-fluoro-2-methoxy-phenyl)-5-hydroxy-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate: To a solution of tert-butyl 2-[5-[tert-butyl(dimethyl)silyl]oxy-4-(4-fluoro-2-methoxy-phenyl)-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate (120 mg, 0.178 mmol) in THF (5 mL) was added tetra-n-butylammonium fluoride (93 mg, 0.36 mmol). The mixture was stirred at rt for 16 h. The mixture was poured into water and the product was extracted with EtOAc. The organics were washed with brine, dried over anhydrous Na₂SO₄, concentrated and purified by preparative TLC (DCM/MeOH: 20/1) to give tert-butyl 2-[4-(4-fluoro-2-methoxy-phenyl)-5-hydroxy-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate (60 mg, 60% yield). LCMS ESI (+) m/z 561.2 (M+H).

Step L: Preparation of tert-butyl 2-[5-fluoro-4-(4-fluoro-2-methoxy-phenyl)-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate: To a solution of tert-butyl 2-[4-(4-fluoro-2-methoxy-phenyl)-5-hydroxy-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate (21 mg, 0.032 mmol) in DCM (2 mL) was added diethylaminosulfurtrifluoride (0.0084 mL, 0.064 mmol). The mixture was stirred at rt for 3 h. The mixture was poured into water and the product was extracted with EtOAc. The organics were washed with brine, dried over anhydrous Na₂SO₄, concentrated and purified by preparative TLC (DCM/MeOH: 20/1) to give tert-butyl 2-[5-fluoro-4-(4-fluoro-2-methoxy-phenyl)-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate (12 mg, 67% yield).

Step M: Preparation of 2-[5-fluoro-4-(4-fluoro-2-methoxy-phenyl)-1-(1-methylpyrazol-4-yl)-6,7-dihydro-5H-cyclopenta [c]pyridin-3-yl]-4,5,6,7-tetrahydropyrazolo[1,5-a]pyrazine: To a solution of tert-butyl 2-[5-fluoro-4-(4-fluoro-2-methoxy-phenyl)-1-(1-methylpyrazol-4-yl)-6,7-dihydro-5H-cyclopenta[c]pyridin-3-yl]-6,7-dihydro-4H-pyrazolo[1,5-a]pyrazine-5-carboxylate (12 mg, 0.021 mmol) in dichloromethane (3 mL) was added trifluoroacetic acid (1 mL). The resulting mixture was stirred at rt for 2 h. The solvent was concentrated under reduced pressure to afford crude 2-[5-fluoro-4-(4-fluoro-2-methoxy-phenyl)-1-(1-methylpyrazol-4-yl)-6,7-dihydro-5H-cyclopenta[c]pyridin-3-yl]-4,5,6,7-tetrahydropyrazolo[1,5-a]pyrazine (15 mg, 100% yield), which was used in the next step without further purification.

Step N: Preparation of 1-[2-[5-fluoro-4-(4-fluoro-2-methoxy-phenyl)-1-(1-methylpyrazol-4-yl)-6,7-dihydro-5H-cyclopenta[c]pyridin-3-yl]-6,7-dihydro-4H-pyrazolo[1,5-a]pyrazin-5-yl]prop-2-en-1-one: 2-[5-fluoro-4-(4-fluoro-2-methoxy-phenyl)-1-(1-methylpyrazol-4-yl)-6,7-dihydro-5H-cyclopenta[c]pyridin-3-yl]-4,5,6,7-tetrahydropyrazolo[1,5-a]pyrazine (15 mg, 0.021 mmol) in THF (1 mL) was added dropwise to a solution of K₂CO₃ (13 mg, 0.097 mmol) in water (1 mL) and ethyl acetate (1 mL). The reaction mixture was stirred at rt for 10 mins. A solution of acryloyl chloride (21 mg, 0.023 mmol) in DCM (0.2 mL) was added to the mixture dropwise at 0° C. The mixture was stirred at 0° C. for 30 min. The reaction was quenched by addition of NaHCO₃ aqueous solution, and was extracted with EtOAc. The organics were then separated and dried (Na₂SO₄) before concentration to dryness. The residue was purified by preparative TLC (DCM/MeOH=10/1) to give 1-[2-[5-fluoro-4-(4-fluoro-2-methoxy-phenyl)-1-(1-methylpyrazol-4-yl)-6,7-dihydro-5H-cyclopenta[c]pyridin-3-yl]-6,7-dihydro-4H-pyrazolo[1,5-a]pyrazin-5-yl]prop-2-en-1-one (5.2 mg, 47% yield). ¹H NMR (400 MHz, CD₃OD) δ 8.35-8.49 (m, 1H), 8.17-8.27 (m, 1H), 7.16-7.28 (m, 1H), 6.66-6.99 (m, 3H), 6.28 (d, J=16.7 Hz, 1H), 5.57-5.98 (m, 3H), 4.09-4.32 (m, 4H), 4.00-4.08 (m, 3H), 3.62 (s, 2H), 3.59 (s, 1H), 3.40-3.50 (m, 1H), 3.11-3.25 (m, 2H), 2.37-2.56 (m, 2H). LCMS ESI (+) m/z 517.1 (M+H).

### Synthetic Example 63: Synthesis of (R)-3-(6-(2-acryloyl-1,2,3,4-tetrahydroisoquinolin-7-yl)-7-(2,4-difluoro-6-(2-methoxyethoxy)phenyl)thieno[3,2-c]pyridin-4-yl)-5,8-dihydro-1,6-naphthyridin-7(6H)-one (Compound 579) and (S)-3-(6-(2-acryloyl-1,2,3,4-tetrahydroisoquinolin-7-yl)-7-(2,4-difluoro-6-(2-methoxyethoxy)phenyl)thieno[3,2-c]pyridin-4-yl)-5,8-dihydro-1,6-naphthyridin-7(6H)-one (Compound 580)

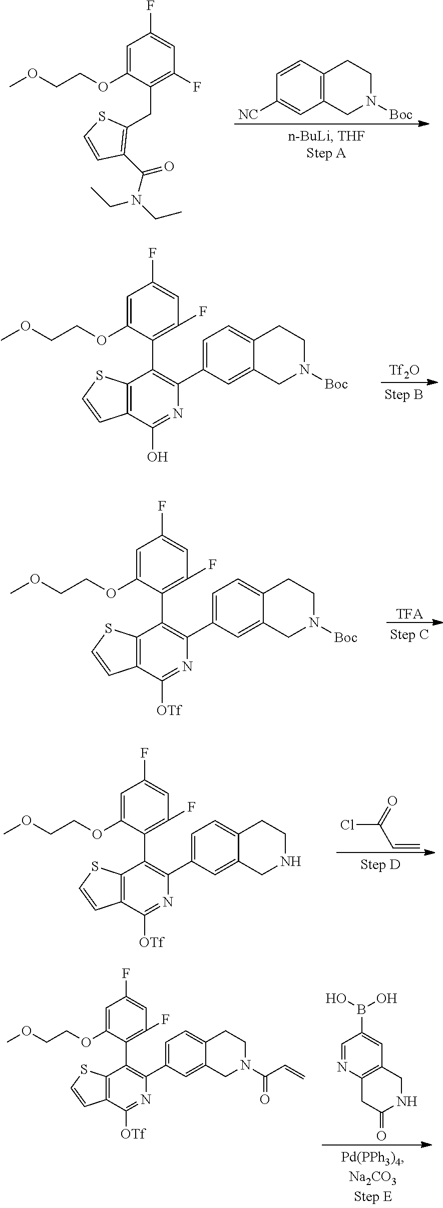

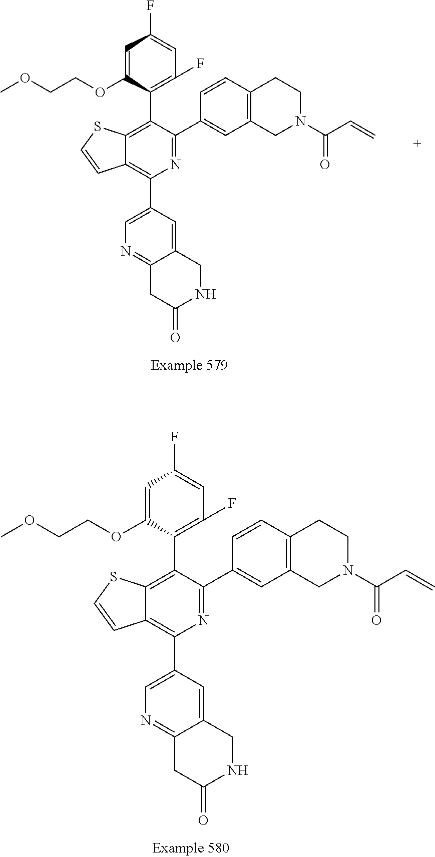

Step A: Preparation of tert-butyl 7-[7-[2,4-difluoro-6-(2-methoxyethoxy)phenyl]-4-hydroxy-thieno[3,2-c]pyridin-6-yl]-3,4-dihydro-1H-isoquinoline-2-carboxylate: To a solution of 2-[[2,4-difluoro-6-(2-methoxyethoxy)phenyl]methyl]-N,N-diethyl-thiophene-3-carboxamide (713 mg, 1.86 mmol) in THF (2 mL) was added n-butyllithium (2.5 M in hexanes, 0.81 mL, 2.0 mmol) at −60° C. under Ar. The solution was then stirred at −60° C. for 20 min. A solution of tert-butyl 7-cyano-3,4-dihydro-1H-isoquinoline-2-carboxylate (400 mg, 1.55 mmol) in THF (0.5 mL) was added. The mixture was warmed to rt and stirred for 3 h. 1 N HCl aqueous solution was added to adjust the pH to 6. The mixture was extracted with EtOAc twice. The combined organic layers were washed with saturated brine solution, dried over anhydrous Na₂SO₄, filtered and concentrated. The resulting residue was purified by column chromatography on silica gel (EtOAc/petroleum ether: 1/1) to give tert-butyl 7-[7-[2,4-difluoro-6-(2-methoxyethoxy)phenyl]-4-hydroxy-thieno[3,2-c]pyridin-6-yl]-3,4-dihydro-1H-isoquinoline-2-carboxylate (573 mg, 65% yield) as a yellow solid. LCMS ESI (+) m/z 568.2 (M+H).

Step B: Preparation of tert-butyl 7-[7-[2,4-difluoro-6-(2-methoxyethoxy)phenyl]-4-(trifluoromethylsulfonyloxy)thieno[3,2-c]pyridin-6-yl]-3,4-dihydro-1H-isoquinoline-2-carboxylate: To a solution of tert-butyl 7-[7-[2,4-difluoro-6-(2-methoxyethoxy)phenyl]-4-hydroxy-thieno[3,2-c]pyridin-6-yl]-3,4-dihydro-1H-isoquinoline-2-carboxylate (573 mg, 1.01 mmol) and pyridine (0.820 mL, 10.1 mmol) in DCM (10 mL)) was added Tf₂O (0.850 g, 3.00 mmol) at 0° C. under Ar. The mixture was stirred at 0° C. for 1 h. The mixture was washed with water, dried over Na₂SO₄ and concentrated. The residue was purified by column chromatography on silica gel (EtOAc/petroleum ether: 1/8) to give tert-butyl 7-[7-[2,4-difluoro-6-(2-methoxyethoxy)phenyl]-4-(trifluoromethylsulfonyloxy)thieno[3,2-c]pyridin-6-yl]-3,4-dihydro-1H-isoquinoline-2-carboxylate (615 mg, 87% yield).

Step C: Preparation of [7-[2,4-difluoro-6-(2-methoxvethoxy)phenyl]-6-(1,2,3,4-tetrahydroisoquinolin-7-yl)thieno[3,2-c]pyridin-4-yl]trifluoromethanesulfonate: To a solution of tert-butyl 7-[7-[2,4-difluoro-6-(2-methoxyethoxy)phenyl]-4-(trifluoromethylsulfonyloxy)thieno[3,2-c]pyridin-6-yl]-3,4-dihydro-1H-isoquinoline-2-carboxylate (615 mg, 0.878 mmol) in DCM (9 mL) was added trifluoroacetic acid (3.0 mL). The mixture was stirred at rt for 2 h. The mixture was concentrated and dried in vacuum to give crude [7-[2,4-difluoro-6-(2-methoxyethoxy)phenyl]-6-(1,2,3,4-tetrahydroisoquinolin-7-yl)thieno[3,2-c]pyridin-4-yl]trifluoromethanesulfonate (901 mg) which was used in the next step without further purification. LCMS ESI (+) m/z 600.9 (M+H).

Step D: Preparation of [7-[2,4-difluoro-6-(2-methoxyethoxy)phenyl]-6-(2-prop-2-enoyl-3,4-dihydro-1H-isoquinolin-7-yl)thieno[3,2-c]pyridin-4-yl]trifluoromethanesulfonate: To a solution of crude [7-[2,4-difluoro-6-(2-methoxyethoxy)phenyl]-6-(1,2,3,4-tetrahydroisoquinolin-7-yl)thieno[3,2-c]pyridin-4-yl]trifluoromethanesulfonate (901 mg, 0.878 mmol) in ethyl acetate/water (8 mL/8 mL) was added NaHCO₃ (1.26 g, 15.0 mmol) to adjust the pH to 8. A solution of acryloyl chloride (95 mg, 1.1 mmol) in DCM (1 mL) was added at 0° C. The mixture was stirred at 0° C. for 1 h. The reaction mixture was diluted with water and extracted with EtOAc twice. The combined organic layers were dried over anhydrous Na₂SO₄, filtered and concentrated. The residue was purified by column chromatography (EtOAc/petroleum ether: 1/5) to give [7-[2,4-difluoro-6-(2-methoxyethoxy)phenyl]-6-(2-prop-2-enoyl-3,4-dihydro-1H-isoquinolin-7-yl)thieno[3,2-c]pyridin-4-yl]trifluoromethanesulfonate (440 mg, 77% yield) as a light-yellow solid. LCMS ESI (+) m/z 654.8 (M+H).

Step E: Preparation of (R)-3-(6-(2-acryloyl-1,2,3,4-tetrahydroisoquinolin-7-yl)-7-(2,4-difluoro-6-(2-methoxyethoxy)phenyl)thieno[3,2-c]pyridin-4-yl)-5,8-dihydro-1,6-naphthyridin-7(6H)-one (Compound 579) and (S)-3-(6-(2-acryloyl-1,2,3,4-tetrahydroisoquinolin-7-yl)-7-(2,4-difluoro-6-(2-methoxyethoxy)phenyl)thieno[3,2-c]pyridin-4-yl)-5,8-dihydro-1,6-naphthyridin-7(6H)-one (Compound 580): To a solution of [7-[2,4-difluoro-6-(2-methoxyethoxy)phenyl]-6-(2-prop-2-enoyl-3,4-dihydro-1H-isoquinolin-7-yl)thieno[3,2-c]pyridin-4-yl]trifluoromethanesulfonate (300 mg, 0.458 mmol) and (7-oxo-6,8-dihydro-5H-1,6-naphthyridin-3-yl)boronic acid (132 mg, 0.687 mmol) in 1,4-dioxane (8 mL)/water (0.8 mL) were added Na₂CO₃ (97 mg, 0.92 mmol)) and tetrakis(triphenylphosphine)palladium (53 mg, 0.046 mmol)). The reaction mixture was stirred at 100° C. for 2 h under Ar. The reaction mixture was filtered and concentrated. The residue was purified by preparative TLC (DCM/MeOH: 25/1) to give 3-[7-[2,4-difluoro-6-(2-methoxyethoxy)phenyl]-6-(2-prop-2-enoyl-3,4-dihydro-1H-isoquinolin-7-yl)thieno[3,2-c]pyridin-4-yl]-6,8-dihydro-5H-1,6-naphthyridin-7-one (206 mg, 69% yield) as a mixture of two diastereomers. 99 mg of the mixture was separated by chiral HPLC (CHIRALPAK IG, 3 cm×25 cm, 5 m, hexane (containing 0.1% DEA):(EtOH:DCM=1:1)=40:60) to give (R)-3-(6-(2-acryloyl-1,2,3,4-tetrahydroisoquinolin-7-yl)-7-(2,4-difluoro-6-(2-methoxyethoxy)phenyl)thieno[3,2-c]pyridin-4-yl)-5,8-dihydro-1,6-naphthyridin-7(6H)-one (Compound 579) (39 mg) as the first peak, and (S)-3-(6-(2-acryloyl-1,2,3,4-tetrahydroisoquinolin-7-yl)-7-(2,4-difluoro-6-(2-methoxyethoxy)phenyl)thieno[3,2-c]pyridin-4-yl)-5,8-dihydro-1,6-naphthyridin-7(6H)-one (Compound 580) (38 mg) as the second peak. Compound 579: ¹H NMR (400 MHz, CD₃OD) δ 9.01 (d, J=2.1 Hz, 1H), 8.30 (s, 1H), 7.82 (d, J=5.7 Hz, 1H), 7.72 (d, J=5.7 Hz, 1H), 7.40 (s, 1H), 7.27-7.33 (m, 1l), 7.11 (d, J=8.1 Hz, 1H), 6.77-6.89 (m, 2H), 6.60-6.68 (m, 1H), 6.19-6.29 (m, 1H), 5.79 (dd, J=1.8, 10.5 Hz, 1H), 4.66-4.78 (m, 4H), 3.95-4.13 (m, 2H), 3.80-3.92 (m, 3H), 3.42-3.54 (m, 2H), 3.13 (s, 3H), 2.86-2.97 (m, 2H); LCMS ESI (+) m/z 653.4 (M+H). Compound 580: ¹H NMR (400 MHz, CD₃OD) δ 9.01 (d, J=2.1 Hz, 1H), 8.30 (s, 1H), 7.82 (d, J=5.7 Hz, 1H), 7.72 (d, J=5.7 Hz, 1H), 7.40 (s, 1H), 7.28-7.34 (m, 1H), 7.10 (d, J=8.1 Hz, 1H), 6.78-6.90 (m, 2H), 6.60-6.69 (m, 1H), 6.21-6.29 (m, 1H), 5.79 (dd, J=2.1, 10.8 Hz, 1H), 4.67-4.79 (m, 4H), 3.96-4.14 (m, 2H), 3.81-3.92 (m, 3H), 3.43-3.54 (m, 2H), 3.13 (s, 3H), 2.86-2.97 (m, 2H); LCMS ESI (+) m/z 653.3 (M+H).

### Synthetic Example 64: Synthesis of 1-[(4S,7S)-2-[7-[2,4-difluoro-6-(2-methoxyethoxy)phenyl]-4-(2-methyl-3,4-dihydro-1H-isoquinolin-6-yl)thieno[3,2-c]pyridin-6-yl]-4,7-dimethyl-6,7-dihydro-4H-pyrazolo[1,5-a]pyrazin-5-yl]prop-2-en-1-one (Compound 601)

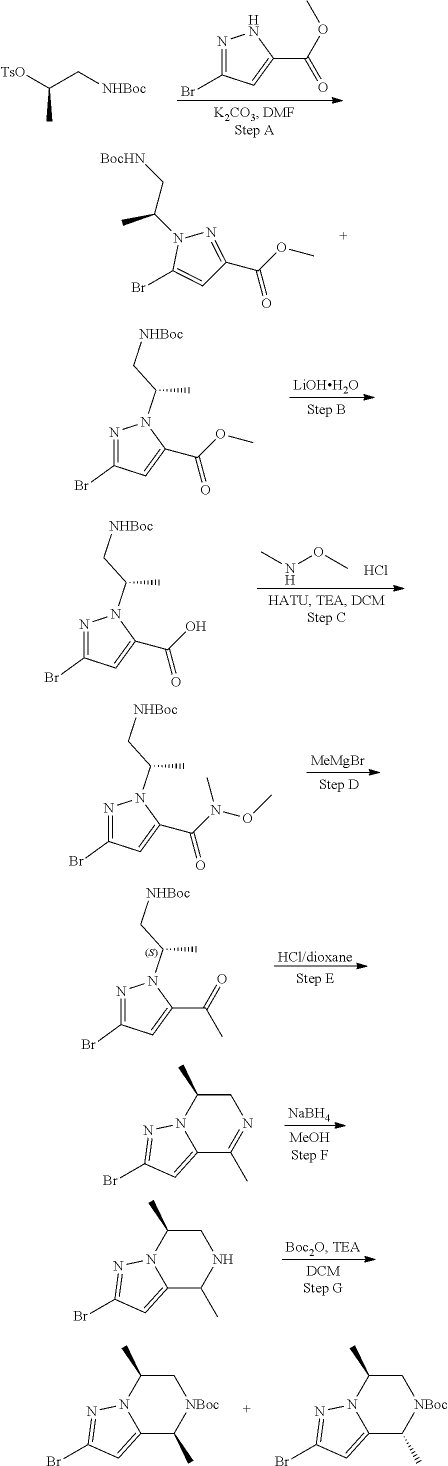

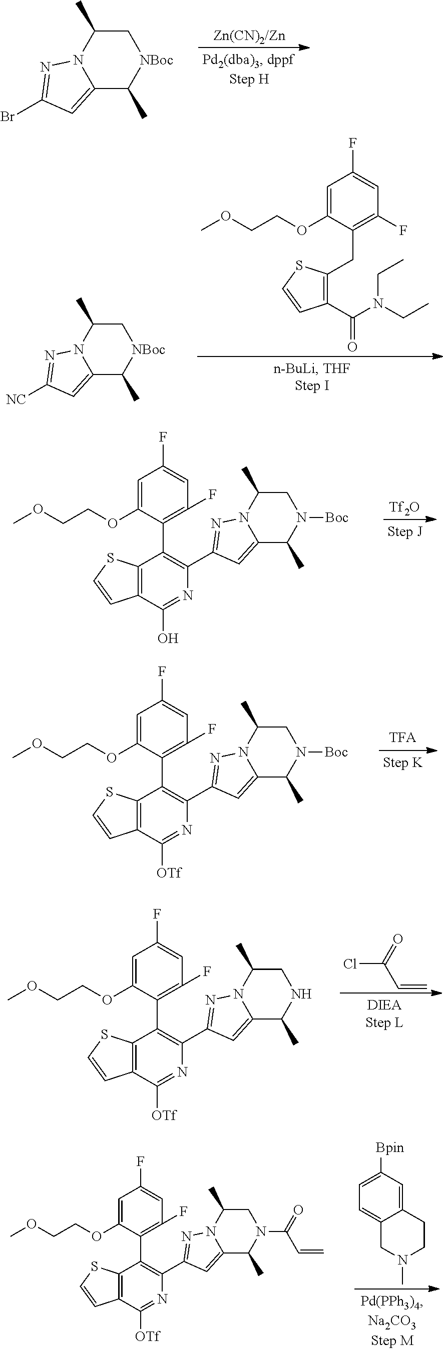

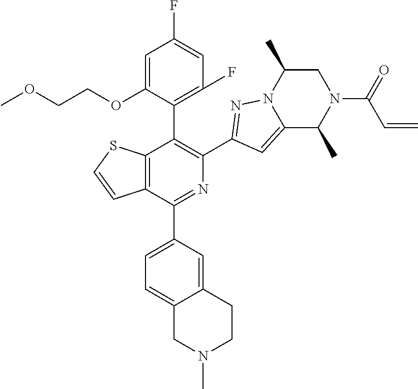 [SMILES: C=CC(=O)N1C[C@H](C)n2nc(-c3nc(-c4ccc5c(c4)CCN(C)C5)c4ccsc4c3-c3c(F)cc(F)cc3OCCOC)cc2[C@@H]1C]

Step A: Preparation of methyl 5-bromo-2-[(1S)-2-(tert-butoxycarbonylamino)-1-methyl-ethyl]pyrazole-3-carboxylate: To a solution of methyl 3-bromo-1H-pyrazole-5-carboxylate (3.00 g, 14.6 mmol) in DMF (50 mL) were added [(1R)-2-(tert-butoxycarbonylamino)-1-methyl-ethyl]4-methylbenzenesulfonate (5.06 g, 15.4 mmol) and K₂CO₃ (4.04 g, 29.3 mmol). The mixture was stirred at 90° C. for 16 hours. The reaction mixture was concentrated and diluted with water. The mixture was extracted with EtOAc, washed with saturated brine, dried and concentrated to afford crude methyl 5-bromo-2-[(1S)-2-(tert-butoxycarbonylamino)-1-methyl-ethyl]pyrazole-3-carboxylate (5.30 g, 100% yield) as a 2:1 mixture of desired methyl 5-bromo-2-[(1S)-2-(tert-butoxycarbonylamino)-1-methyl-ethyl]pyrazole-3-carboxylate and the undesired isomer methyl (S)-5-bromo-1-(1-((tert-butoxycarbonyl)-12-azaneyl)propan-2-yl)-1H-pyrazole-3-carboxylate. The isomers were separated in Step C. LCMS ESI (+) m/z 361.9 (M+H).

Step B: Preparation of 5-bromo-2-[(1S)-2-(tert-butoxycarbonylamino)-1-methyl-ethyl]pyrazole-3-carboxylic acid: To a solution of methyl 5-bromo-2-[(1S)-2-(tert-butoxycarbonylamino)-1-methyl-ethyl]pyrazole-3-carboxylate (5.30 g, 14.6 mmol) in THF (15 mL), methanol (15 mL) and water (15 mL) was added lithium hydroxide (1.40 g, 58.5 mmol). The mixture was stirred at rt for 6 h. 1 N aqueous HCl was added to adjust the pH to 2. The mixture was extracted with EtOAc, dried over anhydrous Na₂SO₄, filtered and concentrated to afford crude 5-bromo-2-[(1S)-2-(tert-butoxycarbonylamino)-1-methyl-ethyl]pyrazole-3-carboxylic acid (3.70 g, 73% yield) as a yellow solid. The crude was used in the next step without further purification. LCMS ESI (+) m/z 348.1 (M+H).

Step C: Preparation of tert-butyl N-[(2S)-2-[3-bromo-5-[methoxy(methyl)carbamoyl]pyrazol-1-yl]propyl]carbamate: To a solution of 5-bromo-2-[(1S)-2-(tert-butoxycarbonylamino)-1-methyl-ethyl]pyrazole-3-carboxylic acid (3.70 g, 10.6 mmol) and N-methoxymethanamine (0.780 g, 12.8 mmol) in DCM (50 mL) were added HATU (6.06 g, 15.9 mmol) and DIEA (5.70 mL, 31.9 mmol). The resulting mixture was stirred at rt for 2 hours. The mixture was poured into water and the product was extracted with DCM. The organic phase was dried over Na₂SO₄, filtered and concentrated. The residue was purified by column chromatography on silica gel (EtOAc:petroleum ether=5:1) to afford tert-butyl N-[(2S)-2-[3-bromo-5-[methoxy(methyl)carbamoyl]pyrazol-1-yl]propyl]carbamate (2.44 g, 59% yield) as a yellow solid. LCMS ESI (+) m/z 391.0 (M+H).

Step D: Preparation of tert-butyl N-[(2S)-2-(5-acetyl-3-bromo-pyrazol-1-yl)propyl]carbamate: To a solution of tert-butyl N-[(2S)-2-[3-bromo-5-[methoxy(methyl)carbamoyl]pyrazol-1-yl]propyl]carbamate (2.44 g, 6.24 mmol) in THF (30 mL) was added dropwise methylmagnesium bromide (1.0 M in THF, 24.9 mL, 24.9 mmol) at 0° C. under N₂ atmosphere. The mixture was stirred at rt for 4 hours. The reaction was quenched with saturated aqueous NH₄Cl solution and diluted with water. The mixture was extracted with EtOAc. The organic phase was dried over anhydrous Na₂SO₄, filtered and concentrated to afford tert-butyl N-[(2S)-2-(5-acetyl-3-bromo-pyrazol-1-yl)propyl]carbamate (1.90 g, 88% yield) as a yellow solid. LCMS ESI (+) m/z 345.9 (M+H).

Step E: Preparation of (7S)-2-bromo-4,7-dimethyl-6,7-dihydropyrazolo[1,5-a]pyrazine: To a solution of tert-butyl N-[(2S)-2-(5-acetyl-3-bromo-pyrazol-1-yl)propyl]carbamate (1.90 g, 5.49 mmol) in 1,4-dioxane (10 mL) was added 4 N HCl in dioxane (10 mL). The mixture was stirred at rt for 4 hours. The mixture was concentrated. Saturated aqueous NaHCO₃ solution was added to adjust the pH to 8. The mixture was extracted with EtOAc. The organic phase was dried over Na₂SO₄, filtered and concentrated to afford crude (7S)-2-bromo-4,7-dimethyl-6,7-dihydropyrazolo[1,5-a]pyrazine (1.40 g) which was used in the next step without further purification. LCMS ESI (+) m/z 227.9 (M+H).

Step F: Preparation of (7S)-2-bromo-4,7-dimethyl-4,5,6,7-tetrahydropyrazolo[1,5-a]pyrazine: To a solution of (7S)-2-bromo-4,7-dimethyl-6,7-dihydropyrazolo[1,5-a]pyrazine (1.40 g, 6.14 mmol) in methanol (15 mL) was added NaBH₄ (929 mg, 24.6 mmol) at 0° C. in small portions. The mixture was stirred at rt for 2 hours, The mixture was quenched with water and extracted with DCM. The combined organic layers were dried over Na₂SO₄, filtered and concentrated to afford crude (7S)-2-bromo-4,7-dimethyl-4,5,6,7-tetrahydropyrazolo[1,5-a]pyrazine (1.18 g, 84% yield) as a mixture of two diastereomers. LCMS ESI (+) m/z 229.9 (M+H).

Step G: Preparation of (4S,7S)-2-bromo-4,7-dimethyl-6,7-dihydro-4H-pyrazolo[1,5-a]pyrazine-5-carboxylate: To a solution of (7S)-2-bromo-4,7-dimethyl-4,5,6,7-tetrahydropyrazolo[1,5-a]pyrazine (1.18 g, 5.13 mmol), triethylamine (1.40 mL, 10.3 mmol) in DCM (20 mL) was added di-tert-butyl dicarbonate (1.80 mL, 7.69 mmol). The mixture was stirred at rt for 2 hours. The mixture was concentrated and purified by column chromatography on silica gel (petroleum ether:EtOAc=6:1) to afford tert-butyl (4S,7S)-2-bromo-4,7-dimethyl-6,7-dihydro-4H-pyrazolo[1,5-a]pyrazine-5-carboxylate (430 mg, 25% yield) and tert-butyl (4R,7S)-2-bromo-4,7-dimethyl-6,7-dihydro-4H-pyrazolo[1,5-a]pyrazine-5-carboxylate (480 mg, 28% yield) as white solid. LCMS ESI (+) m/z 330.0 (M+H).

Step H: Preparation of tert-butyl (4S,7S)-2-cyano-4,7-dimethyl-6,7-dihydro-4H-pyrazolo [1,5-a]pyrazine-5-carboxylate: A mixture of tert-butyl (4S,7S)-2-bromo-4,7-dimethyl-6,7-dihydro-4H-pyrazolo[1,5-a]pyrazine-5-carboxylate (430 mg, 1.30 mmol), zinc (21 mg, 0.33 mmol), zinc cyanide (229 mg, 1.95 mmol), dppf (73 mg, 0.13 mmol) and Pd₂(dba)₃ (60 mg, 0.065 mmol) in DMA (6 mL) was stirred at 120° C. for 6 h under Ar. The mixture was diluted with EtOAc and filtered. The filtrate was washed by brine, dried over Na₂SO₄, filtered and concentrated. The residue was purified by reserve-phase column chromatography to afford tert-butyl (4S,7S)-2-cyano-4,7-dimethyl-6,7-dihydro-4H-pyrazolo[1,5-a]pyrazine-5-carboxylate (300 mg, 83% yield) as a white solid. LCMS ESI (+) m/z 277.1 (M+H).

Step I: Preparation of tert-butyl (4S,7S)-2-[7-[2,4-difluoro-6-(2-methoxyethoxy)phenyl]-4-hydroxy-thieno[3,2-c]pyridin-6-yl]-4,7-dimethyl-6,7-dihydro-4H-pyrazolo[1,5-a]pyrazine-5-carboxylate: To a solution of 2-[[2,4-difluoro-6-(2-methoxyethoxy)phenyl]methyl]-N,N-diethyl-thiophene-3-carboxamide (200 mg, 0.521 mmol) in THF (3 mL) was added n-BuLi (2.5 M in hexanes, 0.22 mL, 0.55 mmol) at −70° C. under Ar atmosphere. The mixture was stirred at −70° C. for 30 minutes. A solution of tert-butyl (4S,7S)-2-cyano-4,7-dimethyl-6,7-dihydro-4H-pyrazolo[1,5-a]pyrazine-5-carboxylate (120 mg, 0.434 mmol) in THF (0.5 mL) was added. The resulting mixture was stirred room temperature for 2 hours. The reaction mixture was poured into saturated aqueous NH₄Cl solution and extracted with EtOAc. The organic phase was dried over anhydrous Na₂SO₄, filtered and concentrated to afford crude tert-butyl (4S,7S)-2-[7-[2,4-difluoro-6-(2-methoxyethoxy)phenyl]-4-hydroxy-thieno[3,2-c]pyridin-6-yl]-4,7-dimethyl-6,7-dihydro-4H-pyrazolo[1,5-a]pyrazine-5-carboxylate (220 mg, 86% yield) as a yellow solid. LCMS ESI (+) m/z 587.0 (M+H).

Step J: Preparation of tert-butyl (4S,7S)-2-[7-[2,4-difluoro-6-(2-methoxyethoxy)phenyl]-4-(trifluoromethylsulfonyloxy)thieno[3,2-c]pyridin-6-yl]-4,7-dimethyl-6,7-dihydro-4H-pyrazolo[1,5-a]pyrazine-5-carboxylate: To a solution of tert-butyl (4S,7S)-2-[7-[2,4-difluoro-6-(2-methoxyethoxy)phenyl]-4-hydroxy-thieno[3,2-c]pyridin-6-yl]-4,7-dimethyl-6,7-dihydro-4H-pyrazolo[1,5-a]pyrazine-5-carboxylate (220 mg, 0.375 mmol) and pyridine (0.300 mL, 3.75 mmol) in DCM (5 mL) was added trifluoromethanesulfonic anhydride (0.095 mL, 0.56 mmol). The resulting mixture was stirred at 0° C. for 2 hours. The solvent was removed under reduced pressure, and the residue was purified by column chromatography on silica gel (petroleum ether:EtOAc=6:1) to afford tert-butyl (4S,7S)-2-[7-[2,4-difluoro-6-(2-methoxyethoxy)phenyl]-4-(trifluoromethylsulfonyloxy)thieno[3,2-c]pyridin-6-yl]-4,7-dimethyl-6,7-dihydro-4H-pyrazolo[1,5-a]pyrazine-5-carboxylate (140 mg, 52% yield) as awhite solid.

Step K: Preparation of [7-[2,4-difluoro-6-(2-methoxyethoxy)phenyl]-6-[rac-(4S,7S)-4,7-dimethyl-4,5,6,7-tetrahydropyrazolo[1,5-a]pyrazin-2-yl]thieno[3,2-c]pyridin-4-yl]trifluoromethanesulfonate: To a solution of tert-butyl (4S,7S)-2-[7-[2,4-difluoro-6-(2-methoxyethoxy)phenyl]-4-(trifluoromethylsulfonyloxy)thieno[3,2-c]pyridin-6-yl]-4,7-dimethyl-6,7-dihydro-4H-pyrazolo[1,5-a]pyrazine-5-carboxylate (140 mg, 0.195 mmol) in DCM (2 mL) was added trifluoroacetic acid (1.0 mL). The resulting solution was stirred at rt for 1 hour. The mixture was concentrated to dryness to afford crude [7-[2,4-difluoro-6-(2-methoxyethoxy)phenyl]-6-[rac-(4S,7S)-4,7-dimethyl-4,5,6,7-tetrahydropyrazolo[1,5-a]pyrazin-2-yl]thieno[3,2-c]pyridin-4-yl]trifluoromethanesulfonate (150 mg) which was used in the next step without further purification. LCMS ESI (+) m/z 618.9 (M+H).

Step L: Preparation of [7-[2,4-difluoro-6-(2-methoxyethoxy)phenyl]-6-[(4S,7S)-4,7-dimethyl-5-prop-2-enoyl-6,7-dihydro-4H-pyrazolo[1,5-a]pyrazin-2-yl]thieno[3,2-c]pyridin-4-yl]trifluoromethanesulfonate: To a solution of [7-[2,4-difluoro-6-(2-methoxyethoxy)phenyl]-6-[(4S,7S)-4,7-dimethyl-4,5,6,7-tetrahydropyrazolo[1,5-a]pyrazin-2-yl]thieno[3,2-c]pyridin-4-yl]trifluoromethanesulfonate (150 mg, 0.242 mmol) and NaHCO₃ (203 mg, 2.42 mmol) in ethyl acetate (3 mL) and water (3 mL) was added acryloyl chloride (0.030 mL, 0.36 mmol) at 0° C. The mixture was stirred at 0° C. for 15 mins. The reaction mixture was poured into saturated NaHCO₃ aqueous solution, and the mixture was extracted with EtOAc. The organic layer was washed with brine, dried over anhydrous sodium sulfate, filtered and concentrated to give [7-[2,4-difluoro-6-(2-methoxyethoxy)phenyl]-6-[(4S,7S)-4,7-dimethyl-5-prop-2-enoyl-6,7-dihydro-4H-pyrazolo[1,5-a]pyrazin-2-yl]thieno[3,2-c]pyridin-4-yl]trifluoromethanesulfonate (120 mg, 74% yield) as a yellow solid. The crude was used in the next step directly. LCMS ESI (+) m/z 672.8 (M+H).

Step M: Preparation of 1-[(4S,7S)-2-[7-[2,4-difluoro-6-(2-methoxyethoxy)phenyl]-4-(2-methyl-3,4-dihydro-1H-isoquinolin-6-yl)thieno[3,2-c]pyridin-6-yl]-4,7-dimethyl-6,7-dihydro-4H-pyrazolo[1,5-a]pyrazin-5-yl]prop-2-en-1-one: To a solution of [7-[2,4-difluoro-6-(2-methoxyethoxy)phenyl]-6-[(4S,7S)-4,7-dimethyl-5-prop-2-enoyl-6,7-dihydro-4H-pyrazolo[1,5-a]pyrazin-2-yl]thieno[3,2-c]pyridin-4-yl]trifluoromethanesulfonate (25 mg, 0.037 mmol), 2-methyl-6-(4,4,5,5-tetramethyl-1,3,2-dioxaborolan-2-yl)-3,4-dihydro-TH-isoquinoline (15 mg, 0.056 mmol) and Na₂CO₃ (0.026 mL, 0.15 mmol) in 1,4-dioxane (1 mL) and water (0.1 mL) was added Pd(PPh₃)₄(4.3 mg, 0.0037 mmol) under nitrogen. The mixture was stirred at 100° C. for 2 hours. The solvent was removed under reduced pressure. The residue was purified by preparative TLC (DCM:MeOH=10:1) and further purified by preparative HPLC to afford 1-[(4S,7S)-2-[7-[2,4-difluoro-6-(2-methoxyethoxy)phenyl]-4-(2-methyl-3,4-dihydro-TH-isoquinolin-6-yl)thieno[3,2-c]pyridin-6-yl]-4,7-dimethyl-6,7-dihydro-4H-pyrazolo[1,5-a]pyrazin-5-yl]prop-2-en-1-one (8.4 mg, 31% yield) as a bis trifluoroacetic acid salt. ¹H NMR (400 MHz, CD₃OD) δ 7.85-7.96 (m, 2H), 7.81 (d, J=5.1 Hz, 1H), 7.66 (d, J=5.8 Hz, 1H), 7.46 (d, J=8.4 Hz, 1H), 6.65-6.92 (m, 3H), 6.47 (s, 1H), 6.19-6.34 (m, 1H), 5.46-5.86 (m, 2H), 4.21-4.81 (m, 4H), 3.71-4.2 (m, 4H), 3.33-3.68 (m, 5H), 3.12 (s, 3H), 3.02 (d, J=2.2 Hz, 3H), 1.33-1.56 (m, 6H). LCMS ESI (+) m/z 670.0 (M+H).

### Synthetic Example 65: Synthesis of 5-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)isoindolin-1-one (Compound 617)

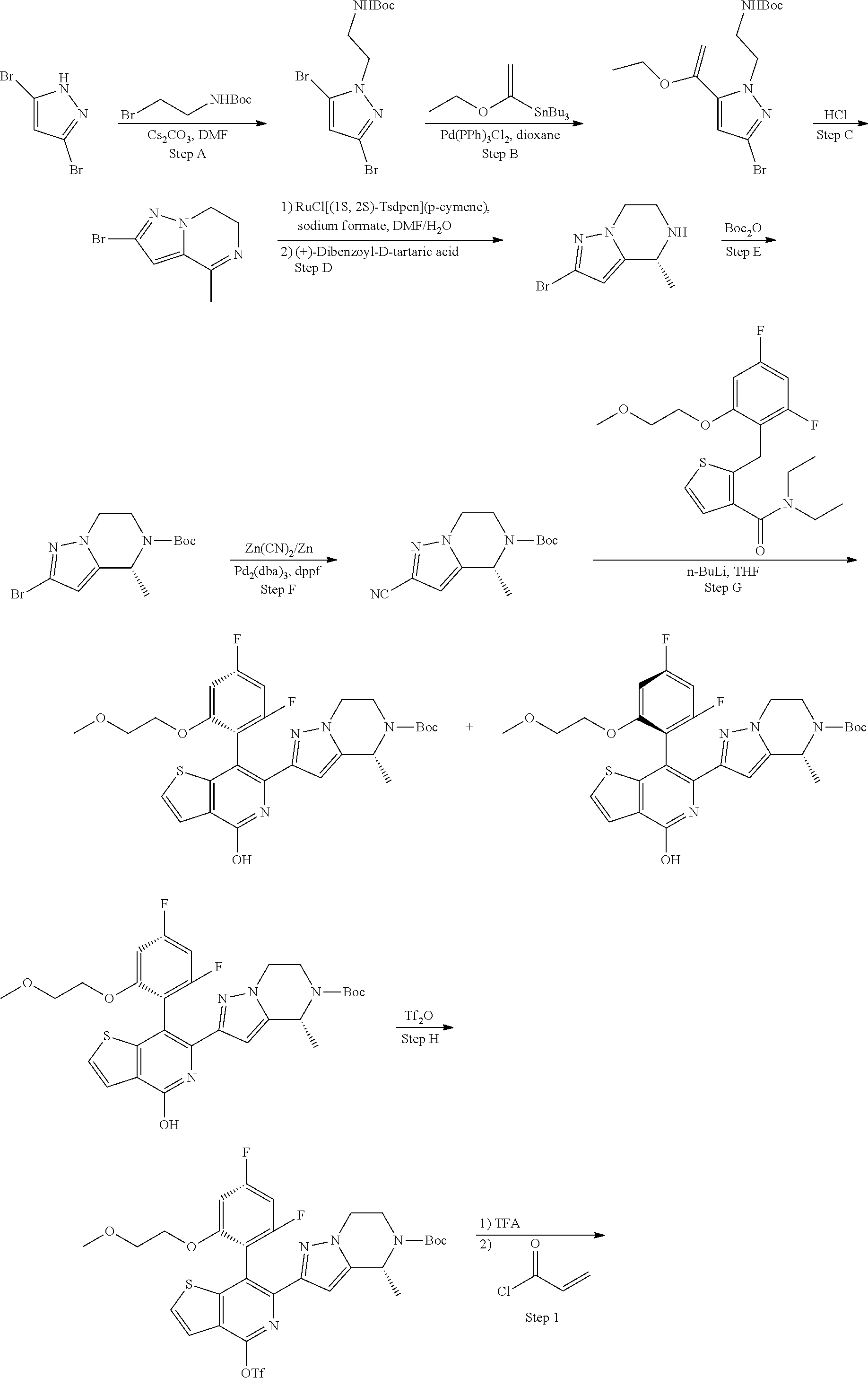

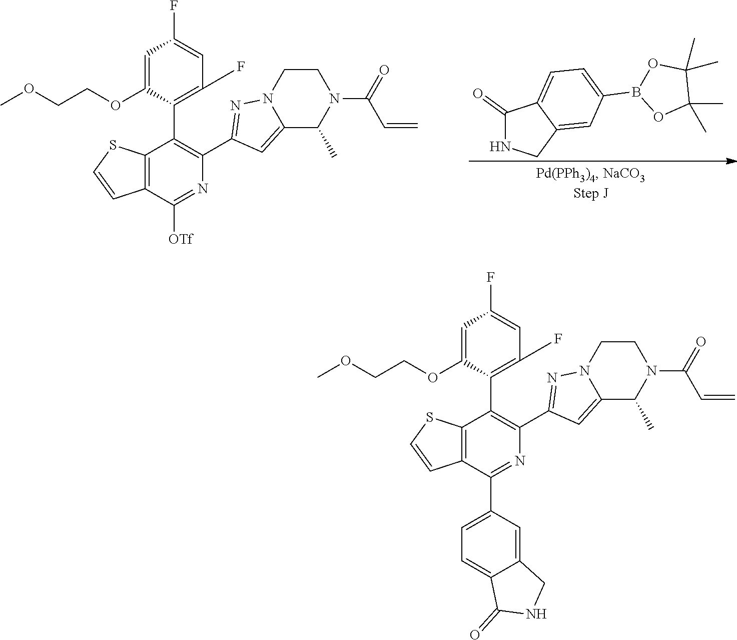

Step A: Preparation of tert-butyl (2-(3,5-dibromo-1H-pyrazol-1-yl)ethyl)carbamate: A solution of 3,5-dibromo-1H-pyrazole (15.0 g, 66.4 mmol) and tert-butyl N-(2-bromoethyl)carbamate (17.1 g, 76.4 mmol) in DMF (10 mL) was added cesium carbonate (32.5 g, 99.6 mmol). The resulting mixture was stirred for 12 h at rt and then concentrated. To the residue was added water. The mixture was extracted with EtOAc, washed with brine, dried over Na₂SO₄, filtered and concentrated under reduced pressure. The crude product tert-butyl (2-(3,5-dibromo-1H-pyrazol-1-yl)ethyl)carbamate (24.0 g, 98% yield) was used in the next step without further purification.

Step B: Preparation of tert-butyl (2-(3-bromo-5-(1-ethoxyvinyl)-1H-pyrazol-1-yl)ethyl)carbamate: A solution of tert-butyl N-[2-(3,5-dibromopyrazol-1-yl)ethyl]carbamate (14.0 g, 37.9 mmol), 1-ethoxyvinyltri-n-butyltin (13.0 mL, 37.9 mmol) and bis(triphenylphosphine)palladium(II) chloride (1.33 g, 1.90 mmol) in 1,4-dioxane (150 mL) was stirred at 100° C. for 4 h under N₂ atmosphere. The mixture was concentrated, then potassium fluoride aqueous solution (200 mL) and EtOAc (200 mL) was added. The mixture was stirred for additional 0.5 h. The organic layer was separated and washed with saturated brine solution, dried over Na₂SO₄ before concentration to dryness. The residue was then purified by column chromatography on silica gel (Petroleum ether/EtOAc=20/1 to 10/1) to give tert-butyl (2-(3-bromo-5-(1-ethoxyvinyl)-1H-pyrazol-1-yl)ethyl)carbamate (9.0 g, 66%) as a yellow oil. LCMS ESI (+) m/z 359 (M+H).

Step C: Preparation of 2-bromo-4-methyl-6,7-dihydropyrazolo[1,5-a]pyrazine: A solution of tert-butyl N-[2-[3-bromo-5-(1-ethoxyvinyl)pyrazol-1-yl]ethyl]carbamate (16.0 g, 44.4 mmol) in dioxane (50 mL) and 4 N HCl in 1,4-dioxane (120 mL) was stirred at rt for 1 h. The reaction was concentrated under reduced pressure. The residue was taken up in DCM and saturated sodium bicarbonate solution was added to adjust the pH to 8. The organics layer was separated and dried over Na₂SO₄ before concentration to dryness to give 2-bromo-4-methyl-6,7-dihydropyrazolo[1,5-a]pyrazine (9.0 g, 95%) which was used in the next step without further purification.

Step D: Preparation of (4R)-2-bromo-4-methyl-4,5,6,7-tetrahydropyrazolo[1,5-a]pyrazine: To a solution of crude 2-bromo-4-methyl-6,7-dihydropyrazolo[1,5-a]pyrazine (250.0 g, 1168 mmol) and HCOONa (317.7 g, 4672 mmol) in DMF (1300 mL) and water (1300 mL) was added RuCl[(1S,2S)-Tsdpen](p-cymene) (14.7 g, 23.4 mmol). The mixture was stirred at 50° C. under argon for 24 h. The mixture was extracted with DCM. The combined organics were dried over Na₂SO₄ before concentration to dryness. The crude was then purified by column chromatography eluting with EtOAc to give 2-bromo-4-methyl-4,5,6,7-tetrahydropyrazolo[1,5-a]pyrazine (136.0 g, 54% yield) as a yellow solid with 80% ee. A solution of crude (4R)-2-bromo-4-methyl-4,5,6,7-tetrahydropyrazolo[1,5-a]pyrazine (76.0 g, 352 mmol) and (+)-dibenzoyl-D-tartaric acid (128.5 g, 359.0 mmol) in EtOAc (4 L) was stirred at 75° C. for 12 h. The reaction was cooled to rt and filtered, and the filter cake was washed with EtOAc (700 mL) to get a white solid. The solid was suspended in EtOAc (4 L) and stirred at 75° C. for 10 h. The reaction was cooled to rt and filtered. The filter cake was washed with EtOAc to get a white solid. The solid was taken up in DCM. Saturated sodium bicarbonate aqueous solution was added to adjust the pH to 9. The mixture was extracted with DCM (3×600 mL). The combined organics were dried over Na₂SO₄, filtered and concentrated to give (4R)-2-bromo-4-methyl-4,5,6,7-tetrahydropyrazolo[1,5-a]pyrazine (52.0 g, 68% yield). LCMS ESI (+) m/z 216 (M+H).

Step E: Preparation of tert-butyl (4R)-2-bromo-4-methyl-6,7-dihydro-4H-pyrazolo[1,5-a]pyrazine-5-carboxylate: To a solution of (4R)-2-bromo-4-methyl-4,5,6,7-tetrahydropyrazolo[1,5-a]pyrazine (136.0 g, 629.4 mmol) in DCM (1000 mL) were added triethylamine (263.0 mL, 1888 mmol) and di-tert-butyl dicarbonate (151.0 g, 692.0 mmol). The result mixture was stirred at rt for 2 h. The mixture was washed with water (1000 mL). The organic phase was dried over Na₂SO₄, filtered and concentrated under reduced pressure. The residue was purified by column chromatography on silica gel (petroleum ether/EtOAc=20/1 to 10/1) to give tert-butyl (4R)-2-bromo-4-methyl-6,7-dihydro-4H-pyrazolo[1,5-a]pyrazine-5-carboxylate (198.0 g, 100% yield). ¹H NMR (400 MHz, CDCl₃) δ 6.06 (s, 1H), 5.20-5.48 (m, 1H), 4.30-4.57 (m, 1H), 3.95-4.20 (m, 2H), 3.21-3.42 (m, 1H), 1.50 (s, 9H), 1.43 (d, J=6.8 Hz, 3H). LCMS ESI (+) m/z 316 (M+H).

Step F: Preparation of tert-butyl (R)-2-cyano-4-methyl-6,7-dihydropyrazolo[1,5-a]pyrazine-5(4H)-carboxylate: A suspension of tert-butyl (4R)-2-bromo-4-methyl-6,7-dihydro-4H-pyrazolo[1,5-a]pyrazine-5-carboxylate (198.0 g, 626.0 mmol), zinc cyanide (103.0 g, 877.0 mmol), zinc (16.38 g, 250.0 mmol), 1,1′-bis(diphenylphosphino) ferrocene (41.66 g, 75.10 mmol) and tris(dibenzylideneacetone) dipalladium(0) (28.67 g, 31.30 mmol) in DMA (1200 mL) was stirred for 14 h at 120° C. under N₂ atmosphere. The mixture was concentrated and diluted with water (2000 mL) and the mixture was extracted with EtOAc (100 mL×2). The combined organics were washed with saturated brine solution. The organics were then separated and dried over Na₂SO₄ before concentration to dryness. The crude was then purified by column chromatography on silica gel (petroleum ether/EtOAc=20/1 to 10/1) followed by reserve-phase column chromatography to give tert-butyl (R)-2-cyano-4-methyl-6,7-dihydropyrazolo[1,5-a]pyrazine-5(4H)-carboxylate (148.0 g, 90% yield) as a light-yellow solid. ¹H NMR (400 MHz, CDCl₃) δ 6.46 (s, 1H), 5.22-5.51 (m, 1H), 4.35-4.60 (m, 1H), 4.20-4.31 (m, 1H), 4.05-4.18 (m, 1H), 3.21-3.42 (m, 1H), 1.50 (s, 9H), 1.45 (d, J=6.8 Hz, 3H). LCMS ESI (+) m/z 263.1 (M+H).

Step G: Preparation of (tert-butyl (R)-2-((S)-7-(2,4-difluoro-6-(2-methoxyethoxy)phenyl)-4-hydroxythieno[3,2-c]pyridin-6-yl)-4-methyl-6,7-dihydropyrazolo[1,5-a]pyrazine-5(4H)-carboxylate: To a solution of 2-[[2,4-difluoro-6-(2-methoxyethoxy)phenyl]methyl]-N,N-diethyl-thiophene-3-carboxamide (155 g, 404 mmol) in THF (1000 mL) was added n-butyllithium (157 mL, 2.5 M in hexanes, 393 mmol) at −60° C. The mixture was stirred at this temperature for 30 mins, and then tert-butyl (R)-2-cyano-4-methyl-6,7-dihydropyrazolo[1,5-a]pyrazine-5(4H)-carboxylate (97.80 g, 373.0 mmol) in THF (200 mL) was added below −50° C. The resulting mixture was stirred at 0° C. for 3 h. Water (1.5 L) was added to quench the reaction. 1 N HCl aqueous solution was added to adjust the pH to 5-6. The product was extracted with EtOAc (1.2 L×3). The combined organic phases were dried over anhydrous Na₂SO₄, filtered and concentrated. The residue was purified by column chromatography on silica gel (EtOAc/petroleum ether: 5/2) three times to give the less polar desired product tert-butyl (R)-2-((S)-7-(2,4-difluoro-6-(2-methoxyethoxy)phenyl)-4-hydroxythieno[3,2-c]pyridin-6-yl)-4-methyl-6,7-dihydropyrazolo[1,5-a]pyrazine-5(4H)-carboxylate (72.0 g, 34% yield) and more polar byproduct tert-butyl (R)-2-((R)-7-(2,4-difluoro-6-(2-methoxyethoxy)phenyl)-4-hydroxythieno[3,2-c]pyridin-6-yl)-4-methyl-6,7-dihydropyrazolo[1,5-a]pyrazine-5(4H)-carboxylate (58.0 g, 27% yield). LCMS ESI (+) m/z 573.2 (M+H).

Step H: Preparation of tert-butyl (R)-2-((S)-7-(2,4-difluoro-6-(2-methoxyethoxy)phenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate: To a solution of tert-butyl (R)-2-((S)-7-(2,4-difluoro-6-(2-methoxyethoxy)phenyl)-4-hydroxythieno[3,2-c]pyridin-6-yl)-4-methyl-6,7-dihydropyrazolo[1,5-a]pyrazine-5(4H)-carboxylate (20.00 g, 34.90 mmol) and pyridine (13.80 g, 174.5 mmol) in DCM (150 mL) was added trifluoromethanesulfonic anhydride (12.0 mL, 69.9 mmol) at 0° C. The mixture was stirred at 0° C. for 1 h. The mixture was poured into water. The product was extracted with DCM, dried over anhydrous Na₂SO₄, filtered and concentrated. The residue was purified by silica gel column chromatography eluting with 20% EtOAc in petroleum ether to give tert-butyl (R)-2-((S)-7-(2,4-difluoro-6-(2-methoxyethoxy)phenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (22.0 g, 89% yield). ¹H NMR (400 MHz, CDCl₃) δ 7.57 (d, J=5.6 Hz, 1H), 7.48 (d, J=5.6 Hz, 1H), 6.54-6.60 (m, 2H), 6.41-6.50 (m, 1H), 5.21-5.40 (m, 1H), 4.25-4.48 (m, 1H), 3.82-4.05 (m, 4H), 3.32-3.39 (m, 2H), 3.15-3.30 (m, 1H), 3.10 (s, 1H), 1.49 (s, 9H), 1.42 (d, J=6.6 Hz, 3H). LCMS ESI (+) m/z 705.2 (M+H).

Step I: Preparation of tert-butyl (4S,7S)-2-[7-[2,4-difluoro-6-(2-methoxyethoxy) phenyl]-4-hydroxy-thieno[3,2-c]pyridin-6-yl]-4,7-dimethyl-6,7-dihydro-4H-pyrazolo[1,5-a]pyrazine-5-carboxylate: To a solution of tert-butyl (R)-2-((S)-7-(2,4-difluoro-6-(2-methoxyethoxy)phenyl)-4-(((trifluoromethyl) sulfonyl)oxy)thieno[3,2-c]pyridin-6-yl)-4-methyl-6,7-dihydropyrazolo[1,5-a]pyrazine-5(4H)-carboxylate (1.04 g, 1.48 mmol) in DCM (6 mL) was added trifluoroacetic acid (2.0 mL). The mixture was stirred at 24° C. for 30 minutes. The mixture was concentrated to dryness. The residue was taken up in ethyl acetate (10 mL) and water (10 mL). NaHCO₃ (1.29 g, 15.3 mmol) was added, followed by a solution of acryloyl chloride (0.160 g, 1.78 mmol) in DCM (2 mL) dropwise at 0° C. The mixture was stirred for 0.5 hour at 0° C. The reaction mixture was diluted with DCM, washed with water and brine and dried (Na₂SO₄) before concentration to dryness. The crude was purified by column chromatography on silica gel eluting with 20% EtOAc in petroleum ether to give tert-butyl (4S,7S)-2-[7-[2,4-difluoro-6-(2-methoxyethoxy)phenyl]-4-hydroxy-thieno[3,2-c]pyridin-6-yl]-4,7-dimethyl-6,7-dihydro-4H-pyrazolo[1,5-a]pyrazine-5-carboxylate (0.750 g, 77% yield over two steps). LCMS ESI (+) m/z 659.1 (M+H).

Step J: Preparation of 5-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)isoindolin-1-one: A mixture of tert-butyl (4S,7S)-2-[7-[2,4-difluoro-6-(2-methoxyethoxy)phenyl]-4-hydroxy-thieno[3,2-c]pyridin-6-yl]-4,7-dimethyl-6,7-dihydro-4H-pyrazolo[1,5-a]pyrazine-5-carboxylate (15 mg, 0.023 mmol), 5-(4,4,5,5-tetramethyl-1,3,2-dioxaborolan-2-yl)isoindolin-1-one (12 mg, 0.046 mmol), sodium carbonate (4.8 mg, 0.046 mmol) and tetrakis(triphenylphosphine)palladium(0) (2.6 mg, 0.0023 mmol) in 1,4-dioxane (2 mL)/water (0.2 mL) was stirred at 100° C. for 2 h. The mixture was filtered and concentrated. The residue was purified by preparative HPLC to afford 5-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)isoindolin-1-one (11 mg, 74% yield) as a trifluoroacetic acid salt. ¹H NMR (400 MHz, CD₃OD) δ 8.21 (s, 1H), 8.12 (dd, J=7.9, 1.5 Hz, 1H), 8.06 (d, J=7.9 Hz, 1H), 7.97 (d, J=5.6 Hz, 1H), 7.76 (d, J=5.6 Hz, 1H), 6.95 (dt, J=11.0, 1.9 Hz, 1H), 6.74-6.87 (m, 2H), 6.29 (d, J=16.6 Hz, 1H), 6.12 (s, 1H), 5.83 (dd, J=11.6, 1.8 Hz, 1H), 5.40-5.75 (m, 1H), 4.64 (s, 2H), 3.95-4.21 (m, 4H), 3.44 (t, J=4.6 Hz, 2H), 3.07 (s, 3H), 1.35-1.56 (m, 3H). LCMS ESI (+) m/z 642.0 (M+H).

### Synthetic Example 66: Synthesis of 1-[(6R)-2-[7-[2,4-difluoro-6-[2-fluoro-1-(fluoromethyl)ethoxy]phenyl]-4-(2-methyl-3,4-dihydro-1H-isoquinolin-6-yl)thieno[3,2-c]pyridin-6-yl]-6-methyl-6,7-dihydro-4H-pyrazolo[1,5-a]pyrazin-5-yl]prop-2-en-1-one (Compound 634)

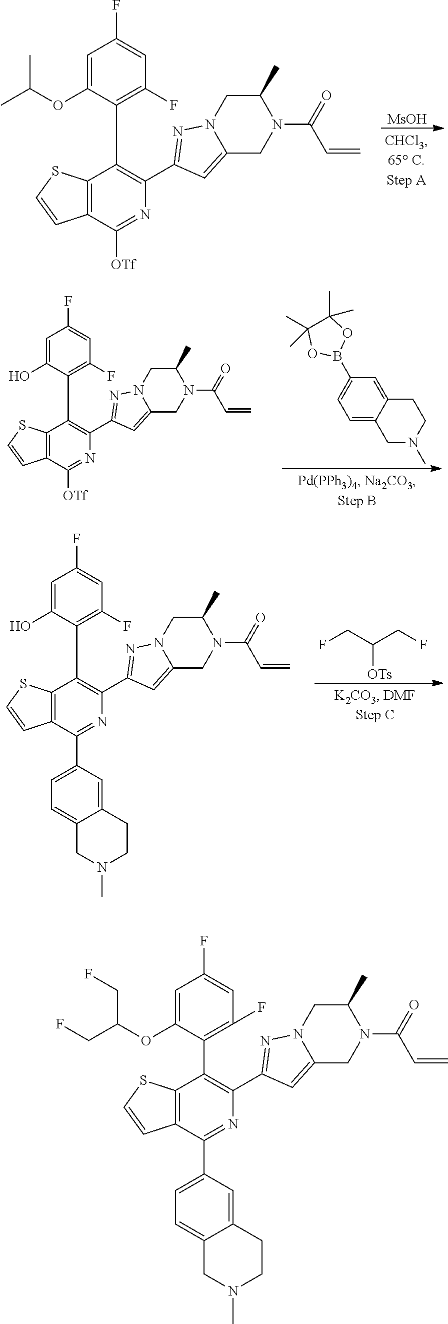

Step A: Preparation of [7-(2,4-difluoro-6-hydroxy-phenyl)-6-[(6R)-6-methyl-5-prop-2-enoyl-6,7-dihydro-4H-pyrazolo[1,5-a]pyrazin-2-yl]thieno[3,2-c]pyridin-4-yl]trifluoromethanesulfonate: To a solution of [7-(2,4-difluoro-6-isopropoxy-phenyl)-6-[(6R)-6-methyl-5-prop-2-enoyl-6,7-dihydro-4H-pyrazolo[1,5-a]pyrazin-2-yl]thieno[3,2-c]pyridin-4-yl]trifluoromethanesulfonate (300 mg, 0.467 mmol) in chloroform (10 mL) was added MsOH (0.91 mL, 14.0 mmol) and the mixture were stirred at 65° C. overnight. The mixture was concentrated under vacuum. The residue was taken up in water, basified with saturated aqueous NaHCO₃ and extracted with DCM. The organics were washed with brine, dried and concentrated. The residue was purified by flash column chromatography eluting with 60% EtOAc in hexanes to get [7-(2,4-difluoro-6-hydroxy-phenyl)-6-[(6R)-6-methyl-5-prop-2-enoyl-6,7-dihydro-4H-pyrazolo[1,5-a]pyrazin-2-yl]thieno[3,2-c]pyridin-4-yl]trifluoromethanesulfonate (230 mg, 82% yield). LCMS ESI (+) m/z 600.6 (M+H).

Step B: Preparation of 1-[(6R)-2-[7-(2,4-difluoro-6-hydroxy-phenyl)-4-(2-methyl-3,4-dihydro-1H-isoquinolin-6-yl)thieno[3,2-c]pyridin-6-yl]-6-methyl-6,7-dihydro-4H-pyrazolo[1,5-a]pyrazin-5-yl]prop-2-en-1-one: A mixture of [7-(2,4-difluoro-6-hydroxy-phenyl)-6-[(6R)-6-methyl-5-prop-2-enoyl-6,7-dihydro-4H-pyrazolo[1,5-a]pyrazin-2-yl]thieno[3,2-c]pyridin-4-yl]trifluoromethanesulfonate (230 mg, 0.383 mmol), 2-methyl-6-(4,4,5,5-tetramethyl-1,3,2-dioxaborolan-2-yl)-3,4-dihydro-1H-isoquinoline (157 mg, 0.574 mmol), Na₂CO₃ (81 mg, 0.77 mmol) and Pd(PPh₃)₄(28 mg, 0.038 mmol) was stirred in 1,4-dioxane (10 mL) and water (1 mL) at 100° C. under N₂ overnight. The reaction was concentrated to dryness and the residue was purified by flash column chromatography eluting with 8% MeOH in DCM to give 1-[(6R)-2-[7-(2,4-difluoro-6-hydroxy-phenyl)-4-(2-methyl-3,4-dihydro-1H-isoquinolin-6-yl)thieno[3,2-c]pyridin-6-yl]-6-methyl-6,7-dihydro-4H-pyrazolo[1,5-a]pyrazin-5-yl]prop-2-en-1-one (170 mg, 74% yield). LCMS ESI (+) m/z 598.1 (M+H).

Step C: Preparation of 1-[(6R)-2-[7-[2,4-difluoro-6-[2-fluoro-1-(fluoromethyl)ethoxy]phenyl]-4-(2-methyl-3,4-dihydro-1H-isoquinolin-6-yl)thieno[3,2-c]pyridin-6-yl]-6-methyl-6,7-dihydro-4H-pyrazolo[1,5-a]pyrazin-5-yl]prop-2-en-1-one: A mixture of 1-[(6R)-2-[7-(2,4-difluoro-6-hydroxy-phenyl)-4-(2-methyl-3,4-dihydro-1H-isoquinolin-6-yl)thieno[3,2-c]pyridin-6-yl]-6-methyl-6,7-dihydro-4H-pyrazolo[1,5-a]pyrazin-5-yl]prop-2-en-1-one (20 mg, 0.034 mmol), [2-fluoro-1-(fluoromethyl)ethyl]4-methylbenzenesulfonate (17 mg, 0.067 mmol) and K₂CO₃ (9.2 mg, 0.067 mmol) was stirred in DMF (5 mL) at 90° C. for 12 h. The reaction was concentrated to dryness and the residue was taken up in EtOAc. The mixture was washed with water and saturated brine solution. The organics were then separated and dried (MgSO₄) before concentration to dryness. The crude was purified by flash column chromatography eluting with 5% MeOH in DCM followed by preparative HPLC (ACN-H₂O (0.1% TFA)=25-60%) to give 1-[(6R)-2-[7-[2,4-difluoro-6-[2-fluoro-1-(fluoromethyl)ethoxy]phenyl]-4-(2-methyl-3,4-dihydro-1H-isoquinolin-6-yl)thieno[3,2-c]pyridin-6-yl]-6-methyl-6,7-dihydro-4H-pyrazolo[1,5-a]pyrazin-5-yl]prop-2-en-1-one (1.4 mg, 5% yield) as a trifluoroacetic acid salt. ¹HNMR (400 MHz, CD₃OD) δ 7.95 (d, J=7.6 Hz, 2H), 7.80 (d, J=5.5 Hz, 1H), 7.69 (d, J=5.6 Hz, 1H), 7.46 (d, J=8.0 Hz, 1H), 7.00 (s, 1H), 6.80 (t, J=9.2 Hz, 2H), 6.28 (dd, J=24.1, 9.1 Hz, 2H), 5.83 (d, J=10.7 Hz, 1H), 5.16 (s, 2H), 4.52 (s, 1H), 4.49-4.38 (m, 2H), 4.33 (dd, J=16.8, 7.7 Hz, 2H), 4.23 (d, J=5.0 Hz, 1H), 4.17-4.07 (m, 1H), 3.50 (s, 1H), 3.45-3.35 (m, 2H), 3.15 (s, 3H), 1.31 (s, 3H). LCMS ESI (+) m/z 675.7 (M+H).

### Synthetic Example 67: Synthesis of 1-((S)-2-((S)-7-(2,4-difluoro-6-(2-hydroxyethoxy)phenyl)-4-(1-methyl-1H-indazol-5-yl)thieno[3,2-c]pyridin-6-yl)-4-methyl-6,7-dihydropyrazolo[1,5-a]pyrazin-5(4H)-yl)prop-2-en-1-one (Compound 663)

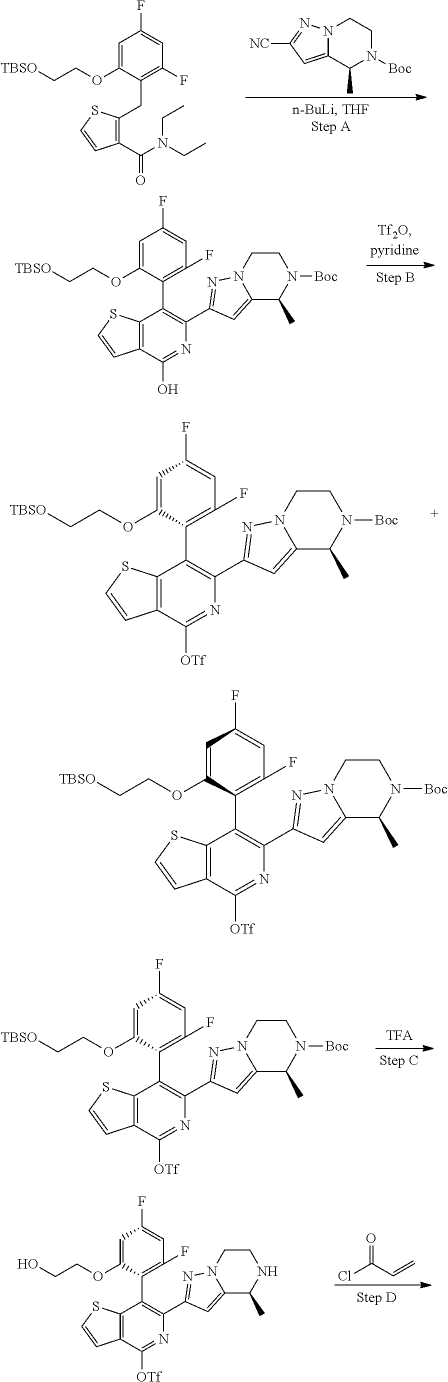

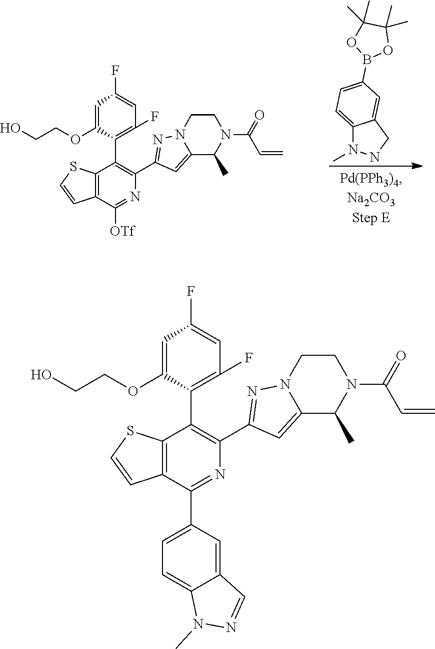

Step A: Preparation of tert-butyl (4S)-2-[7-[2-[2-[tert-butyl(dimethyl)silyl]oxyethoxy-4,6-difluoro-phenyl]-4-hydroxy-thieno[3,2-c]pyridin-6-yl]-4-methyl-67-dihydro-4H-pyrazolo[1,5-a]pyrazine-5-carboxylate: To a solution of 2-[[2-[2-[tert-butyl(dimethyl)silyl]oxyethoxy]-4,6-difluoro-phenyl]methyl]-N,N-diethyl-thiophene-3-carboxamide (2.74 g, 5.66 mmol) in TH (10 mL) as added n-BuLi (2.5 M in hexanes, 2.47 mL, 6.18 mmol) dropwise under Ar. The mixture was stirred at −60° C. for 0.5 h under Ar. Then tert-butyl (4S)-2-cyano-4-methyl-6,7-dihydro-4H-pyrazolo[1,5-a]pyrazine-5-carboxylate (1.49 g, 5.66 mmol) was added at −60° C. The temperature was allowed to warm to, and the resulting mixture was stirred at rt for additional 2 h. The mixture was poured into water. 1 N HCl aqueous solution was added to adjust the pH to 5. The mixture was concentrated. The residue was partitioned between water and EtOAc. The organic phase was separated, dried over anhydrous Na₂SO₄, filtered and concentrated. The residue was purified by column chromatography on silica gel (EtOAc/petroleum ether: 1/2 to 2/1) to give the tert-butyl (4S)-2-[7-[2-[2-[tert-butyl(dimethyl)silyl)oxyethoxy)-4,6-difluoro-phenyl]-4-hydroxy-thieno[3,2-c]pyridin-6-yl]-4-methyl-6,7-dihydro-4H-pyrazolo [1,5-a]pyrazine-5-carboxylate (2.90 g, 53%˜yield). LCMS ESI (+) m/z 673.3 (M+H).

Step B: Preparation of tert-butyl (S)-2-((S)-7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(48)-carboxylate: To a solution of tert-butyl (4S)-2-[7-[2-[2-[tert-butyl(dimethyl)silyl]oxyethoxy]-4,6-difluoro-phenyl]-4-hydroxy-thieno[3,2-c]pyridin-6-yl]-4-methyl-6,7-dihydro-4H-pyrazolo[1,5-a]pyrazine-5-carboxylate (5.40 g, 8.03 mmol) and pyridine (5.06 g, 64.0 mmol) in DCM (25 mL) was added trifluoromethanesulfonic anhydride (5.70 g, 20.2 mmol). The mixture was stirred at 0° C. for 15 min. The mixture was poured into water and the product was extracted with DCM. The organics were washed with brine, dried over anhydrous Na₂SO₄, filtered and concentrated under reduced pressure. The residue was purified by column chromatography on silica gel (EtOAc/petroleum ether: 1/15 to 1/10) to give the desired tert-butyl (S)-2-((S)-7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (1.37 g, 21% yield) as the less polar product, and the undesired diastereomer tert-butyl (S)-2-((R)-7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (1.20 g, 19% yield) as the more polar product.

Step C: Preparation of (S)-7-(2,4-difluoro-6-(2-hydroxyethoxy)phenyl)-6-((S)-4-methyl-4,5,6,7-tetrahydropyrazolo[1,5-a]pyrazin-2-yl)thieno[3,2-c]pyridin-4-yl trifluoromethanesulfonate: To a solution of tert-butyl (S)-2-((S)-7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (1.37 g, 1.70 mmol) in DCM (12 mL) was added trifluoroacetic acid (6.0 mL). The resulting mixture was stirred at room temperature for 2 h. The solvent was concentrated under reduced pressure to afford crude (S)-7-(2,4-difluoro-6-(2-hydroxyethoxy)phenyl)-6-((S)-4-methyl-4,5,6,7-tetrahydropyrazolo[1,5-a]pyrazin-2-yl)thieno[3,2-c]pyridin-4-yl trifluoromethanesulfonate (2.2 g) which was used in the next step without further purification.

Step D: Preparation of (4R)-2-bromo-4-methyl-4,5,6,7-tetrahydropyrazolo[1,5-a]pyrazine: A solution of [7-[2,4-difluoro-6-(2-hydroxyethoxy)phenyl]-6-[(4S)-4-methyl-4,5,6,7-tetrahydropyrazolo[1,5-a]pyrazin-2-yl]thieno[3,2-c]pyridin-4-yl]trifluoromethanesulfonate (2.2 g, 1.7 mmol) in ethyl acetate (10 mL) and water (10 mL) was stirred at 0° C. for 2 mins. NaHCO₃ was added to adjust the pH to 8 followed by the addition of acryloyl chloride (0.19 g, 2.0 mmol) at 0° C. The reaction mixture was stirred at 0° C. for 15 mins. The reaction was diluted with water and extracted with EtOAc. The organics were washed with brine, dried (Na₂SO₄) and concentrated to give crude (S)-6-((S)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate (1.3 g) which was used in the next step without further purification.

Step E: Preparation of 1-((S)-2-((S)-7-(2,4-difluoro-6-(2-hydroxyethoxy)phenyl)-4-(1-methyl-1H-indazol-5-yl)thieno[3,2-c]pyridin-6-yl)-4-methyl-6,7-dihydropyrazolo[1,5-a]pyrazin-5(4H)-yl)prop-2-en-1-one: A solution of crude (S)-6-((S)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate (200 mg, 0.310 mmol), (1-methylindazol-5-yl)boronic acid (66 mg, 0.37 mmol), Pd(PPh₃)₄ (34 mg, 0.030 mmol) and Na₂CO₃ (99 mg, 0.93 mmol) in 1,4-dioxane (2 mL) and water (0.2 mL) was stirred at 100° C. for 2 h under Ar. The mixture was then poured into water and extracted with EtOAc. The organic phase was dried over anhydrous Na₂SO₄, filtered and concentrated. The residue was purified by preparative TLC (DCM:MeOH=15:1) to give 1-((S)-2-((S)-7-(2,4-difluoro-6-(2-hydroxyethoxy)phenyl)-4-(1-methyl-1H-indazol-5-yl)thieno[3,2-c]pyridin-6-yl)-4-methyl-6,7-dihydropyrazolo[1,5-a]pyrazin-5(4H)-yl)prop-2-en-1-one (102 mg, 50% yield) as a white solid. ¹H NMR (400 MHz, CD₃OD) δ 8.27 (s, 1H), 8.06 (s, 1H), 7.95-8.01 (m, 1H), 7.55-7.68 (m, 3H), 6.68-6.79 (m, 2H), 6.56-6.65 (m, 1H), 6.13-6.23 (m, 1H), 6.09 (s, 1H), 5.65-5.76 (m, 1H), 5.23-5.64 (m, 1H), 3.96-4.21 (m, 5H), 3.8-3.95 (m, 3H), 3.37-3.7 (m, 3H), 1.27-1.44 (m, 3H). LCMS ESI (+) m/z 627.0 (M+H).

### Synthetic Example 68: Synthesis of 1-((S)-2-((S)-7-(2,4-difluoro-6-(2-hydroxyethoxy)phenyl)-4-(1-methyl-1H-benzo[d]imidazol-5-yl)thieno[3,2-c]pyridin-6-yl)-4-methyl-6,7-dihydropyrazolo[1,5-a]pyrazin-5(4H)-yl)prop-2-en-1-one (Compound 664)

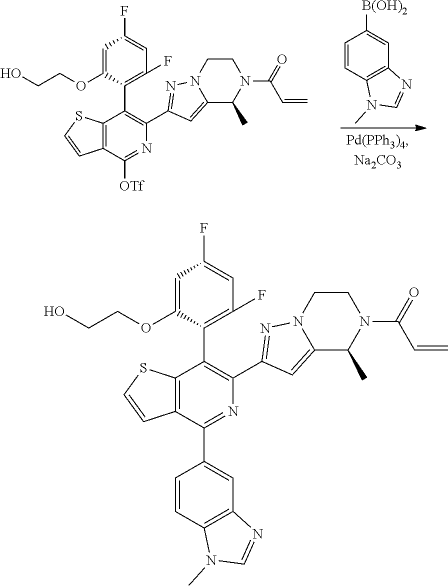

To a solution of (S)-6-((S)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate (13 mg, 0.020 mmol), 1-methyl-5-(4,4,5,5-tetramethyl-1,3,2-dioxaborolan-2-yl)benzimidazole (7.8 mg, 0.033 mmol) and Na₂CO₃ (0.014 mL, 0.081 mmol) in 1,4-dioxane (1 mL) and water (0.1 mL) was added Pd(PPh₃)₄ (2.3 mg, 0.0020 mmol) under nitrogen. The resulting mixture was stirred at 100° C. for 2 hours. The mixture was filtered and purified by preparative HPLC to afford 1-((S)-2-((S)-7-(2,4-difluoro-6-(2-hydroxyethoxy)phenyl)-4-(1-methyl-1H-benzo[d]imidazol-5-yl)thieno[3,2-c]pyridin-6-yl)-4-methyl-6,7-dihydropyrazolo[1,5-a]pyrazin-5(4H)-yl)prop-2-en-1-one (5.1 mg, 39% yield) as a bis trifluoroacetic acid salt. ¹H NMR (400 MHz, CD₃OD) δ 9.32 (s, 1H), 8.45 (s, 1H), 8.28 (d, J=8.6 Hz, 1H), 8.12 (d, J=8.6 Hz, 1H), 7.9 (d, J=5.64 Hz, 1H), 7.75 (d, J=5.64 Hz, 1H), 6.70-6.97 (m, 3H), 6.1-6.41 (m, 2H), 5.41-5.92 (m, 2H), 4.34-4.79 (m, 1H), 4.19-4.31 (m, 4H), 3.91-4.17 (m, 3H), 3.47-3.84 (m, 3H), 1.46 (s, 3H). LCMS ESI (+) m/z 627.2 (M+H).

### Synthetic Example 69: Synthesis of 1-((R)-2-((S)-7-(2,4-difluoro-6-(2-methoxyethoxy)phenyl)-4-(2-methyl-2H-indazol-5-yl)thieno[3,2-c]pyridin-6-yl)-6-methyl-6,7-dihydropyrazolo[1,5-a]pyrazin-5(4H)-yl)prop-2-en-1-one (Compound 668)

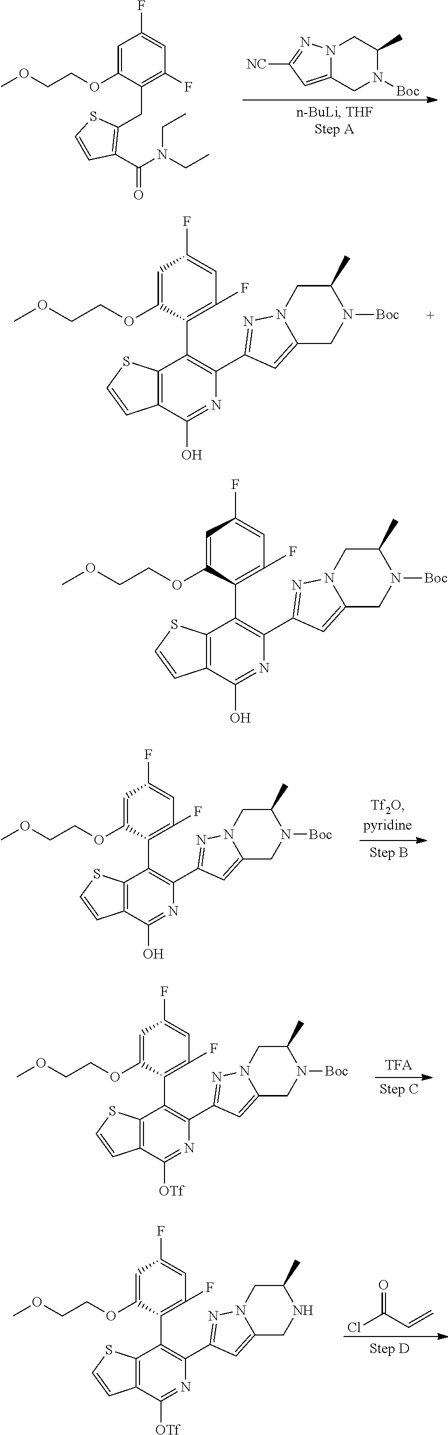

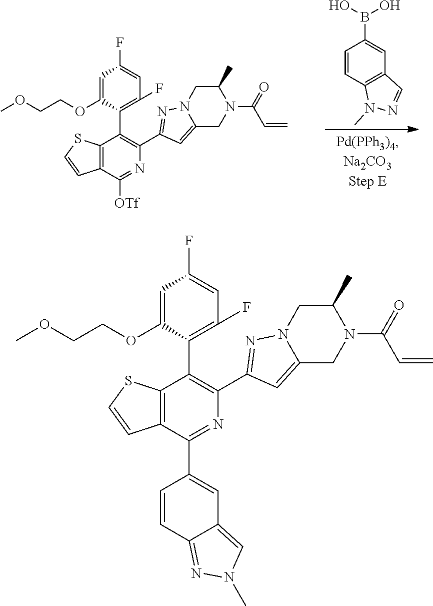

Step A: Preparation of tert-butyl (R)-2-((S)-7-(2,4-difluoro-6-(2-methoxyethoxy)phenyl)-4-hydroxythieno[3,2-c]pyridin-6-yl)-6-methyl-6,7-dihydropyrazolo[1,5-a]pyrazine-5(4H)-carboxylate: To a solution of 2-[[2,4-difluoro-6-(2-methoxyethoxy)phenyl]methyl]-N,N-diethyl-thiophene-3-carboxamide (4.37 g, 11.4 mmol) in THF (60 mL) was added n-butyllithium (2.5 M in hexanes, 4.96 mL, 12.4 mmol) at −60° C. The mixture was stirred at this temperature for 20 mins, and then tert-butyl (6R)-2-cyano-6-methyl-6,7-dihydro-4H-pyrazolo[1,5-a]pyrazine-5-carboxylate (2.60 g, 9.91 mmol) in THF (2 mL) was added. The resulting mixture was stirred rt for 3 h. The mixture was poured into water. 1 N HCl solution was added to adjust the pH to 5. The product was extracted with EtOAc, dried over anhydrous Na₂SO₄, filtered, and concentrated. The residue was purified by column chromatography on silica gel (EtOAc/petroleum ether: 3/1) to give tert-butyl (6R)-2-[7-[2,4-difluoro-6-(2-methoxyethoxy)phenyl]-4-hydroxy-thieno[3,2-c]pyridin-6-yl]-6-methyl-6,7-dihydro-4H-pyrazolo[1,5-a]pyrazine-5-carboxylate (4.49 g, 79% yield) as a mixture of two diastereomers. The mixture was separated by chiral SFC (CHIRAL ART Cellulose-SC, 3 cm×25 cm, 5 m, CO₂:MeOH=60:40) to give tert-butyl (R)-2-((S)-7-(2,4-difluoro-6-(2-methoxyethoxy)phenyl)-4-hydroxythieno[3,2-c]pyridin-6-yl)-6-methyl-6,7-dihydropyrazolo[1,5-a]pyrazine-5(4H)-carboxylate (1.74 g) as the first peak, and tert-butyl (R)-2-((R)-7-(2,4-difluoro-6-(2-methoxyethoxy)phenyl)-4-hydroxythieno[3,2-c]pyridin-6-yl)-6-methyl-6,7-dihydropyrazolo[1,5-a]pyrazine-5(4H)-carboxylate (1.90 g) as the second peak, tert-butyl (R)-2-((S)-7-(2,4-difluoro-6-(2-methoxyethoxy)phenyl)-4-hydroxythieno[3,2-c]pyridin-6-yl)-6-methyl-6,7-dihydropyrazolo[1,5-a]pyrazine-5(4H)-carboxylate: ¹H NMR (400 MHz, CD₃OD) δ 9.91-10.0 (m, 1H), 7.69 (d, J=5.2 Hz, 1H), 7.25 (d, J=5.2 Hz, 1H), 6.62-6.68 (m, 2H), 4.71-4.90 (m, 2H), 3.95-4.28 (m, 5H), 3.40-3.48 (m, 2H), 3.09 (s, 3H), 1.62 (s, 9H), 1.15-1.19 (m, 3H). LCMS ESI (+) m/z 573.0 (M+H).

Step B: Preparation of tert-butyl (R)-2-((S)-7-(2,4-difluoro-6-(2-methoxyethoxy)phenyl)-4-hydroxythieno[3,2-c]pyridin-6-yl)-6-methyl-6,7-dihydropyrazolo[1,5-a]pyrazine-5(4H)-carboxylate: To a mixture of tert-butyl (R)-2-((S)-7-(2,4-difluoro-6-(2-methoxyethoxy)phenyl)-4-hydroxythieno[3,2-c]pyridin-6-yl)-6-methyl-6,7-dihydropyrazolo[1,5-a]pyrazine-5(4H)-carboxylate (502 mg, 0.877 mmol) and pyridine (0.71 mL, 8.8 mmol) in DCM (5 mL) cooled to 0° C. was added trifluoromethanesulfonic anhydride (0.300 mL, 1.75 mmol) under Ar. The mixture was stirred for 20 minutes. The mixture was diluted with EtOAc, washed with water and brine, dried (Na₂SO₄) and concentrated. The crude was purified by flash chromatography on silica gel eluting with 10% EtOAc in petroleum ether to give tert-butyl (R)-2-((S)-7-(2,4-difluoro-6-(2-methoxyethoxy)phenyl)-4-hydroxythieno[3,2-c]pyridin-6-yl)-6-methyl-6,7-dihydropyrazolo[1,5-a]pyrazine-5(4H)-carboxylate (551 mg, 89% yield).

Step C: Preparation of (S)-7-(2,4-difluoro-6-(2-methoxyethoxy)phenyl)-6-((R)-6-methyl-4,5,6,7-tetrahydropyrazolo[1,5-a]pyrazin-2-yl)thieno[3,2-c]pyridin-4-yl trifluoromethanesulfonate: To a solution of tert-butyl (R)-2-((S)-7-(2,4-difluoro-6-(2-methoxyethoxy)phenyl)-4-hydroxythieno[3,2-c]pyridin-6-yl)-6-methyl-6,7-dihydropyrazolo[1,5-a]pyrazine-5(4H)-carboxylate (551 mg, 0.782 mmol) in DCM (6 mL) was added trifluoroacetic acid (2.00 mL, 26.0 mmol) under Ar. The mixture was stirred at 24° C. for 0.5 hour. The mixture was concentrated to give the crude product (685 mg, 100% yield) which was used in the next step without further purification. LCMS ESI (+) m/z 604.8 (M+H).

Step D: Preparation of (S)-6-((R)-5-acryloyl-6-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate: To a solution of (S)-7-(2,4-difluoro-6-(2-methoxyethoxy)phenyl)-6-((R)-6-methyl-4,5,6,7-tetrahydropyrazolo[1,5-a]pyrazin-2-yl)thieno[3,2-c]pyridin-4-yl trifluoromethanesulfonate (685 mg, 0.782 mmol) in ethyl acetate (10 mL) was added water (10 mL) and stirred for 10 minutes at 0° C. NaHCO₃ (0.380 g, 4.53 mmol) was added, followed by acryloyl chloride (0.088 mL, 1.1 mmol) at 0° C. The mixture was stirred at 0° C. for 0.5 hour. The organic layer was separated, dried (Na₂SO₄), filtered and concentrated to dryness to give crude (S)-6-((R)-5-acryloyl-6-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate (516 mg, 100% yield) which was used in the next step without further purification.

Step E: Preparation of 1-((R)-2-((S)-7-(2,4-difluoro-6-(2-methoxyethoxy)phenyl)-4-(2-methyl-2H-indazol-5-yl)thieno[3,2-c]pyridin-6-yl)-6-methyl-6,7-dihydropyrazolo[1,5-a]pyrazin-5(4H)-yl)prop-2-en-1-one: To a solution of (S)-6-((R)-5-acryloyl-6-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate (30 mg, 0.046 mmol) and (2-methylindazol-5-yl)boronic acid (12 mg, 0.068 mmol) in 1,4-dioxane (2 mL) and water (0.2 mL) were added tetrakis(triphenylphosphine)palladium(0) (5.3 mg, 0.0046 mmol) and sodium carbonate (9.7 mg, 0.091 mmol). The mixture was stirred at 100° C. for 2 h under Ar. The mixture was filtered and concentrated. The residue was purified by preparative HPLC to give 1-((R)-2-((S)-7-(2,4-difluoro-6-(2-methoxyethoxy)phenyl)-4-(2-methyl-2H-indazol-5-yl)thieno[3,2-c]pyridin-6-yl)-6-methyl-6,7-dihydropyrazolo[1,5-a]pyrazin-5(4H)-yl)prop-2-en-1-one (17 mg, 60% yield) as a trifluoroacetic acid salt. ¹H NMR (400 MHz, CD₃OD) δ 8.54 (s, 1H), 8.48 (s, 1H), 8.15 (d, J=5.6 Hz, 1H), 7.84-7.97 (m, 3H), 6.98-7.05 (m, 1H), 6.73-6.90 (m, 2H), 6.28 (dd, J=16.6, 1.4 Hz, 1H), 6.08 (m, 1H), 5.82 (dd, J=10.7, 1.2 Hz, 1H), 5.05-5.26 (m, 1H), 4.25-4.42 (m, 6H), 4.03-4.20 (m, 2H), 3.46 (t, J=4.5 Hz, 2H), 3.11 (s, 3H), 1.21 (d, J=6.6 Hz, 3H). LCMS ESI (+) m/z 641.0 (M+H).

### Synthetic Example 70: Synthesis of 1-((R)-2-((S)-7-(2,4-difluoro-6-(2-methoxyethoxy)phenyl)-4-(1-methyl-1H-indazol-5-yl)thieno[3,2-c]pyridin-6-yl)-4-methyl-6,7-dihydropyrazolo[1,5-a]pyrazin-5(4H)-yl)prop-2-en-1-one (Compound 758)

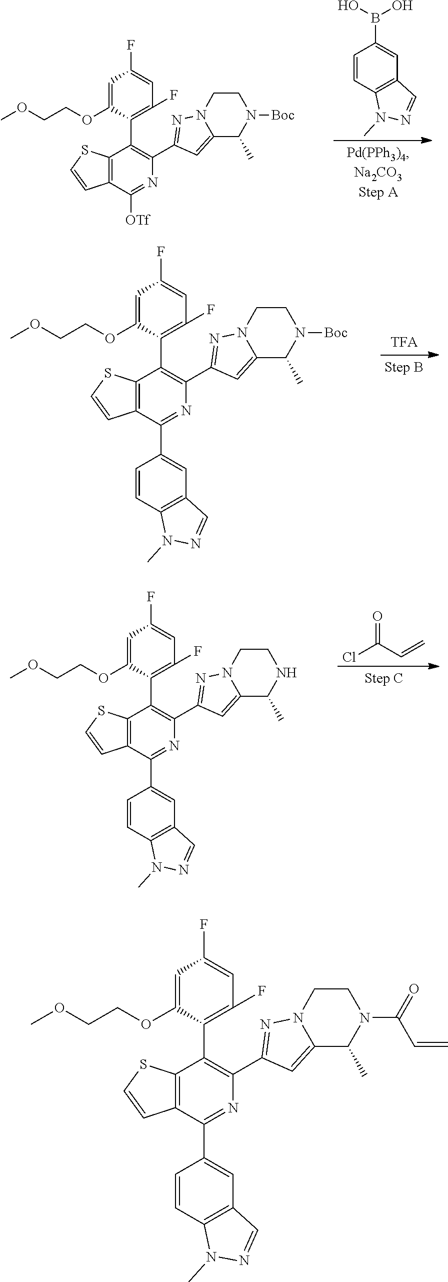

Step A: Preparation of tert-butyl (R)-2-((S)-7-(2,4-difluoro-6-(2-methoxyethoxy)phenyl)-4-(1-methyl-1H-indazol-5-yl)thieno[3,2-c]pyridin-6-yl)-4-methyl-6,7-dihydropyrazolo[1,5-a]pyrazine-5(4H)-carboxylate: To a solution of tert-butyl (R)-2-((S)-7-(2,4-difluoro-6-(2-methoxyethoxy)phenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (5.00 g, 7.10 mmol) and (1-methylindazol-5-yl)boronic acid (1.50 g, 8.51 mmol) in 1,4-dioxane (50 mL)/water (5 mL) was added Na₂CO₃ (1.50 g, 14.2 mmol) and tetrakis(triphenylphosphine)palladium (0.410 g, 0.355 mmol). The reaction mixture was stirred at 100° C. for 3 h under Ar. The reaction mixture was filtered and concentrated. The residue was purified by column chromatography on silica gel (ethyl acetate:petroleum ether=1:1) to give tert-butyl (R)-2-((S)-7-(2,4-difluoro-6-(2-methoxyethoxy)phenyl)-4-(1-methyl-1H-indazol-5-yl)thieno[3,2-c]pyridin-6-yl)-4-methyl-6,7-dihydropyrazolo[1,5-a]pyrazine-5(4H)-carboxylate (4.0 g, 82% yield). LCMS ESI (+) m/z 687.3 (M+H).

Step B: Preparation of (S)-7-(2,4-difluoro-6-(2-methoxyethoxy)phenyl)-4-(1-methyl-1H-indazol-5-yl)-6-((R)-4-methyl-4,5,6,7-tetrahydropyrazolo[1,5-a]pyrazin-2-yl)thieno[3,2-c]pyridine 2,2,2-trifluoroacetate: To a solution of tert-butyl (R)-2-((S)-7-(2,4-difluoro-6-(2-methoxyethoxy)phenyl)-4-(1-methyl-1H-indazol-5-yl)thieno[3,2-c]pyridin-6-yl)-4-methyl-6,7-dihydropyrazolo[1,5-a]pyrazine-5(4H)-carboxylate (50 mg, 0.073 mmol) in DCM (1 mL) was added trifluoroacetic acid (0.5 mL). The mixture was stirred at rt for 2 h. The solution was concentrated to dryness to give (S)-7-(2,4-difluoro-6-(2-methoxyethoxy)phenyl)-4-(1-methyl-1H-indazol-5-yl)-6-((R)-4-methyl-4,5,6,7-tetrahydropyrazolo[1,5-a]pyrazin-2-yl)thieno[3,2-c]pyridine 2,2,2-trifluoroacetate (62 mg, crude), which was used in the next step without further purification. LCMS ESI (+) m/z 587.3 (M+H).

Step C: Preparation 1-((R)-2-((S)-7-(2,4-difluoro-6-(2-methoxyethoxy)phenyl)-4-(1-methyl-1H-indazol-5-yl)thieno[3,2-c]pyridin-6-yl)-4-methyl-6,7-dihydropyrazolo[1,5-a]pyrazin-5(4H)-yl)prop-2-en-1-one: To a solution of (S)-7-(2,4-difluoro-6-(2-methoxyethoxy)phenyl)-4-(1-methyl-1H-indazol-5-yl)-6-((R)-4-methyl-4,5,6,7-tetrahydropyrazolo[1,5-a]pyrazin-2-yl)thieno[3,2-c]pyridine 2,2,2-trifluoroacetate (62 mg crude, 0.073 mmol) in ethyl acetate/water (2 mL/2 mL) was added Na₂CO₃ aqueous solution to adjust the pH to 8. Sodium bicarbonate (9.0 mg, 0.11 mmol) was added. The mixture was cooled to 0° C. A solution of acryloyl chloride (0.0070 mL, 0,086 mmol) in DCM (0.1 mL) was added and the mixture was stirred at 0° C. for 1 h. The reaction mixture was diluted with water and extracted with EtOAc twice. The combined organic layers were dried over anhydrous Na₂SO₄, filtered and concentrated. The residue was purified by preparative TLC (DCM:MeOH=20:1) to give 1-((R)-2-((S)-7-(2,4-difluoro-6-(2-methoxyethoxy)phenyl)-4-(1-methyl-1H-indazol-5-yl)thieno[3,2-c]pyridin-6-yl)-4-methyl-6,7-dihydropyrazolo[1,5-a]pyrazin-5(4H)-yl)prop-2-en-1-one (42 mg, 86% yield) as a white solid ¹H NMR (400 MHz, CD₃OD) δ 8.49 (d, J=1.4 Hz, 1H), 8.27 (s, 1H), 8.01-8.16 (m, 2H), 7.82-7.97 (m, 2H), 7.01 (d, J=10.7 Hz, 1H), 6.76-6.90 (m, 2H), 6.30 (d, J=16.8 Hz, 1H), 6.05 (s, 1H), 5.83 (dd, J=1.7, 10.6 Hz, 1H), 5.41-5.76 (m, 1H), 4.08-4.39 (m, 6H), 3.46 (t, J=4.5 Hz, 2H), 3.09 (s, 3H), 1.35-1.53 (m, 3H). LCMS ESI (+) m/z 641.0 (M+H).

### Synthetic Example 71: Synthesis of 1-((S)-2-((S)-7-(2,4-difluoro-6-((R)-2-hydroxypropoxy)phenyl)-4-(2-methyl-2H-indazol-6-yl)thieno[3,2-c]pyridin-6-yl)-4-methyl-6,7-dihydropyrazolo[1,5-a]pyrazin-5(4H)-yl)prop-2-en-1-one (Compound 770)

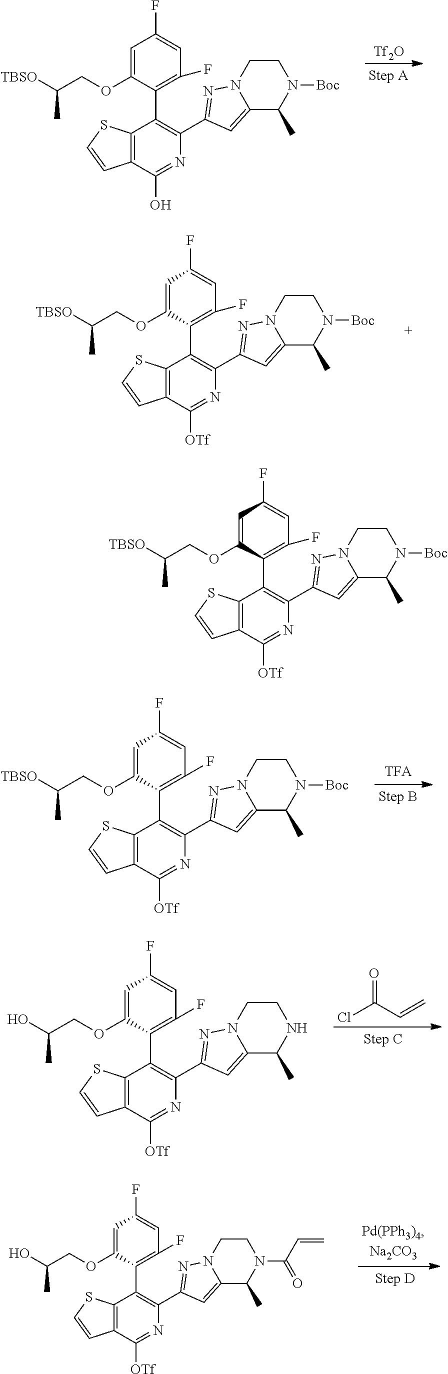

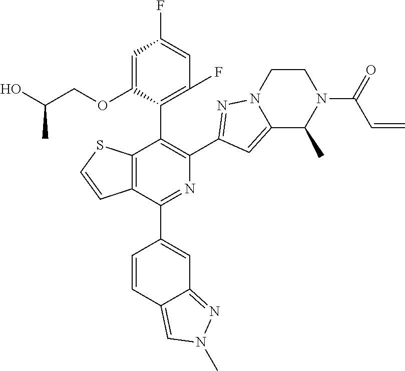

Step A: Preparation of tert-butyl (S)-2-((S)-7-(2-((R)-2-((tert-butyldimethylsilyl)oxy)propoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate: To a mixture of tert-butyl (4S)-2-(7-(2-((R)-2-((tert-butyldimethylsilyl)oxy)propoxy)-4,6-difluorophenyl)-4-hydroxythieno[3,2-c]pyridin-6-yl)-4-methyl-6,7-dihydropyrazolo[1,5-a]pyrazine-5(4H)-carboxylate (720 mg, 1.05 mmol) and pyridine (0.85 mL, 11 mmol) in DCM (10 mL) cooled to 0° C. was added trifluoromethanesulfonic anhydride (0.270 mL, 1.57 mmol) under Ar. The mixture was stirred for 20 minutes. The mixture was diluted with EtOAc, washed with water and saturated brine solution, dried (Na₂SO₄) and concentrated. The crude was purified by flash column chromatography on silica gel eluting with 5% EtOAc in petroleum ether to give the less-polar desired product tert-butyl (S)-2-((S)-7-(2-((R)-2-((tert-butyldimethylsilyl)oxy)propoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (203 mg, 24% yield) and 415 mg of mixture of diastereomers.

Step B: Preparation of (S)-7-(2,4-difluoro-6-((R)-2-hydroxypropoxy)phenyl)-6-((S)-4-methyl-4,5,6,7-tetrahydropyrazolo[1,5-a]pyrazin-2-yl)thieno[3,2-c]pyridin-4-yl trifluoromethanesulfonate: To a solution of tert-butyl (S)-2-((S)-7-(2-((R)-2-((tert-butyldimethylsilyl)oxy)propoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (203 mg, 0.249 mmol) in DCM (3 mL) was added trifluoroacetic acid (1 mL) under Ar. The mixture was stirred at rt for 2 hours. The mixture was concentrated to give crude (S)-7-(2,4-difluoro-6-((R)-2-hydroxypropoxy)phenyl)-6-((S)-4-methyl-4,5,6,7-tetrahydropyrazolo[1,5-a]pyrazin-2-yl)thieno[3,2-c]pyridin-4-yl trifluoromethanesulfonate (235 mg) which was used in the next step without further purification. LCMS ESI (+) m/z 604.9 (M+H).

Step C: Preparation of (S)-6-((S)-5-acryloyl-4-methyl-4,5,6,7-tetrahydropyrazolo[1,5-a]pyrazin-2-yl)-7-(2,4-difluoro-6-((R)-2-hydroxypropoxy)phenyl)thieno[3,2-c]pyridin-4-yl trifluoromethanesulfonate: To a solution of (S)-7-(2,4-difluoro-6-((R)-2-hydroxypropoxy)phenyl)-6-((S)-4-methyl-4,5,6,7-tetrahydropyrazolo[1,5-a]pyrazin-2-yl)thieno[3,2-c]pyridin-4-yl trifluoromethanesulfonate (235 mg, 0.249 mmol) in ethyl acetate (5 mL) was added water (5 mL) and stirred for 10 minutes. The solution was cooled to 0° C. NaHCO₃ (131 mg, 1.55 mmol) was added, followed by acryloyl chloride (33 mg, 0.37 mmol) in DCM (1 mL) dropwise. The mixture was stirred at 0° C. for 1 hour. The organic phase was separated, washed with water and brine, dried and concentrated. The crude was purified by preparative TLC (petroleum ether:EtOAc=4:1) to give (S)-6-((S)-5-acryloyl-4-methyl-4,5,6,7-tetrahydropyrazolo[1,5-a]pyrazin-2-yl)-7-(2,4-difluoro-6-((R)-2-hydroxypropoxy)phenyl)thieno[3,2-c]pyridin-4-yl trifluoromethanesulfonate (153 mg, 93% yield).

Step D: Preparation of 1-((S)-2-((S)-7-(2,4-difluoro-6-((R)-2-hydroxypropoxy)phenyl)-4-(2-methyl-2H-indazol-6-yl)thieno[3,2-c]pyridin-6-yl)-4-methyl-6,7-dihydropyrazolo[1,5-a]pyrazin-5(4H)-yl)prop-2-en-1-one: To a solution of (S)-6-((S)-5-acryloyl-4-methyl-4,5,6,7-tetrahydropyrazolo[1,5-a]pyrazin-2-yl)-7-(2,4-difluoro-6-((R)-2-hydroxypropoxy)phenyl)thieno[3,2-c]pyridin-4-yl trifluoromethanesulfonate (20 mg, 0.030 mmol), 2-methyl-6-(4,4,5,5-tetramethyl-1,3,2-dioxaborolan-2-yl)indazole (16 mg, 0.061 mmol) and Na₂CO₃ (9.7 mg, 0.091 mmol) in 1,4-dioxane (2 mL) and water (0.2 mL) was added Pd(PPh₃)₄(3.3 mg, 0.0046 mmol). The mixture was stirred at 100° C. for 4 h under Ar. The mixture was poured into water and the product was extracted with EtOAc. The organics were washed with brine, dried over anhydrous Na₂SO₄, concentrated and purified by preparative HPLC to give 1-((S)-2-((S)-7-(2,4-difluoro-6-((R)-2-hydroxypropoxy)phenyl)-4-(2-methyl-2H-indazol-6-yl)thieno[3,2-c]pyridin-6-yl)-4-methyl-6,7-dihydropyrazolo[1,5-a]pyrazin-5(4H)-yl)prop-2-en-1-one (8.8 mg, 43% yield) as a trifluoroacetic acid salt. ¹H NMR (400 MHz, CD₃OD) δ 8.41 (s, 1H), 8.28 (s, 1H), 8.01-8.12 (m, 2H), 7.84 (d, J=5.6 Hz, 1H), 7.66 (d, J=8.9 Hz, 1H), 6.95 (d, J=10.9 Hz, 1H), 6.74-6.89 (m, 2H), 6.23-6.35 (m, 1H), 6.07 (s, 1H), 5.83 (d, J=12.0 Hz, 1H), 5.35-5.75 (m, 1H), 4.05-4.56 (m, 6H), 3.69-3.92 (m, 3H), 1.35-1.53 (m, 3H), 0.95 (d, J=6.1 Hz, 3H). LCMS ESI (+) m/z 641.0 (M+H).

### Synthetic Example 72: Synthesis of 1-[(6R)-2-[7-[2,4-difluoro-6-(2-methoxyethoxy)phenyl]-4-(4-dimethylphosphorylphenyl)thieno[3,2-c]pyridin-6-yl]-6-methyl-6,7-dihydro-4H-pyrazolo[1,5-a]pyrazin-5-yl]prop-2-en-1-one (Compound 800)

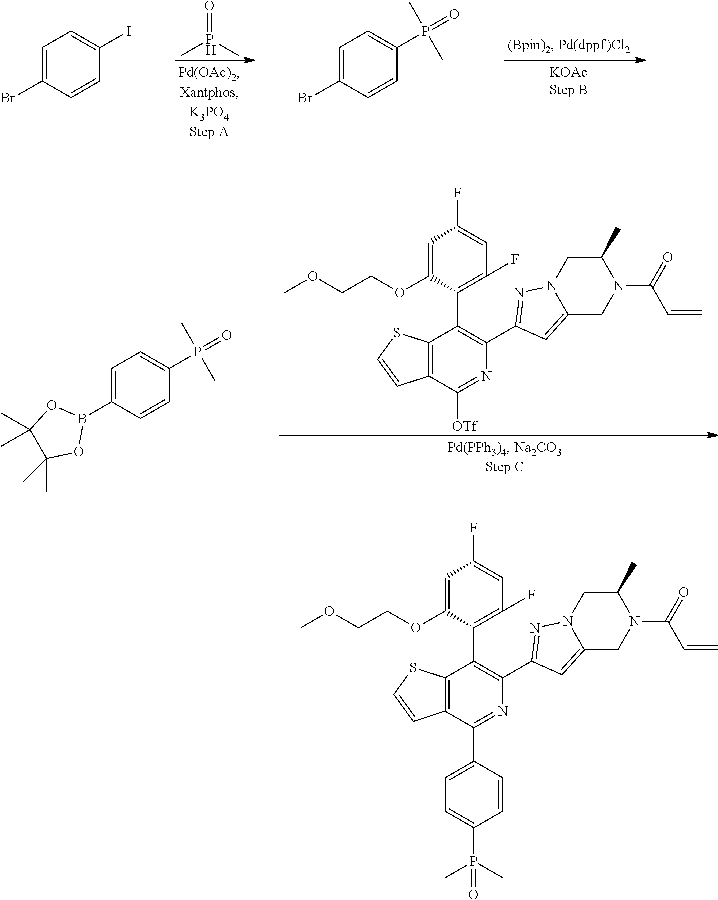

Step A: Preparation of 1-bromo-4-dimethylphosphoryl-benzene: A solution of 1-bromo-4-iodobenzene (282 mg, 1.00 mmol), methylphosphonoylmethane (86 mg, 1.1 mmol), Xantphos (34 mg, 0.060 mmol), Pd(OAc)₂ (11 mg, 0.050 mmol) and K₃PO₄ (232 mg, 1.10 mmol) in DMF (10 mL) was stirred at 100° C. for 16 h. The mixture was concentrated under reduced pressure to afford crude 1-bromo-4-dimethylphosphoryl-benzene (250 mg, 70% yield) as a yellow oil, which was used in the next step directly. LCMS ESI (+) m/z 232.8 (M+H).

Step B: Preparation of 2-(4-dimethylphosphorylphenyl)-4,4,5,5-tetramethyl-1,3,2-dioxaborolane: A mixture of 1-bromo-4-dimethylphosphoryl-benzene (200 mg, 0.858 mmol), (BPin)₂ (327 mg, 1.29 mmol), Pd(dppf)Cl₂(62 mg, 0.086 mmol) and KOAc (169 mg, 1.72 mmol) in 1,4-dioxane (5 mL) was stirred at 80° C. for 16 h under N₂. The reaction was concentrated to dryness and purified by flash column chromatography eluting with 15% EtOAc in hexanes to give 2-(4-dimethylphosphorylphenyl)-4,4,5,5-tetramethyl-1,3,2-dioxaborolane (300 mg, 75% yield) as a yellow oil. LCMS ESI (+) m/z 280.9 (M+H).

Step C: Preparation of 1-[(6R)-2-[7-[2,4-difluoro-6-(2-methoxyethoxy)phenyl]-4-(4-dimethylphosphorylphenyl)thieno[3,2-c]pyridin-6-yl]-6-methyl-6,7-dihydro-4H-pyrazolo[1,5-a]pyrazin-5-yl]prop-2-en-1-one: A solution of [7-[2,4-difluoro-6-(2-methoxyethoxy)phenyl]-6-[(6R)-6-methyl-5-prop-2-enoyl-6,7-dihydro-4H-pyrazolo[1,5-a]pyrazin-2-yl]thieno[3,2-c]pyridin-4-yl]trifluoromethanesulfonate (30 mg, 0.046 mmol), 2-(4-dimethyl phosphorylphenyl)-4,4,5,5-tetramethyl-1,3,2-dioxaborolane (19 mg, 0.068 mmol), Na₂CO₃ (14 mg, 0.14 mmol) and Pd(PPh₃)₄(5.3 mg, 0.0046 mmol) in 1,4-dioxane (5 mL) and water (0.5 mL) was stirred under N₂ at 100° C. for 1 h. The reaction was concentrated to dryness. The crude was purified by flash column chromatography eluting with 5% MeOH in DCM to afford 1-[(6R)-2-[7-[2,4-difluoro-6-(2-methoxyethoxy)phenyl]-4-(4-dimethylphosphorylphenyl)thieno[3,2-c]pyridin-6-yl]-6-methyl-6,7-dihydro-4H-pyrazolo[1,5-a]pyrazin-5-yl]prop-2-en-1-one (7.0 mg, 22% yield). ¹H NMR (400 MHz, CD₃OD) δ 8.17 (dd, J=8.2, 2.4 Hz, 2H), 8.02 (dd, J=11.6, 8.2 Hz, 2H), 7.77 (d, J=5.6 Hz, 1H), 7.68 (d, J=5.6 Hz, 1H), 6.87-6.67 (m, 3H), 6.31-6.24 (m, 2H), 5.81 (dd, J=10.6, 1.8 Hz, 1H), 5.14 (d, J=25.8 Hz, 2H), 4.60 (s, 1H), 4.22 (s, 1H), 4.12-4.05 (m, 2H), 4.03-3.97 (m, 1H), 3.42 (t, J=4.6 Hz, 2H), 3.03 (s, 3H), 1.88 (d, J=13.4 Hz, 6H), 1.28 (s, 3H). LCMS ESI (+) m/z 663.2 (M+H).

### Synthetic Example 73: Synthesis of 6-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)isoindolin-1-one (Compound 845)

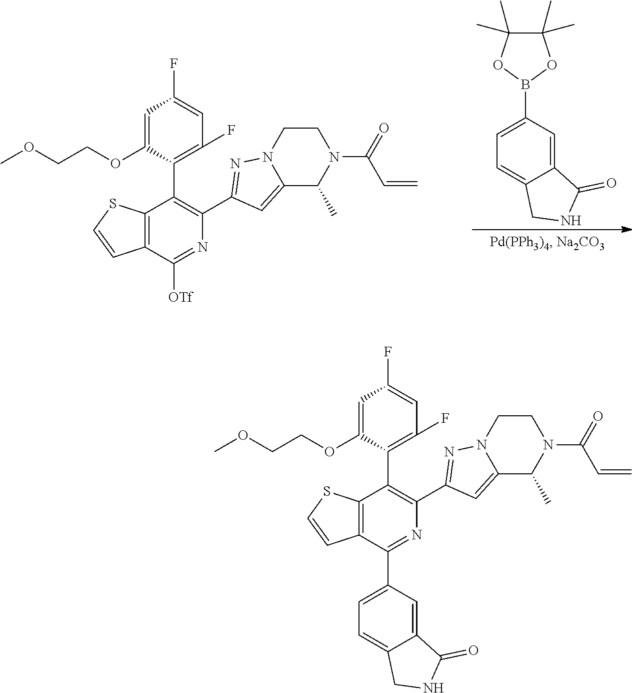

To a solution of (S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate (15 mg, 0.023 mmol), 6-(4,4,5,5-tetramethyl-1,3,2-dioxaborolan-2-yl)isoindolin-1-one (8.9 mg, 0.034 mmol) and Na₂CO₃ (4.9 mg, 0.046 mmol) in 1,4-dioxane (0.5 mL)/water (0.1 mL) was added Pd(PPh₃)₄(2.6 mg, 0.0023 mmol). The resulting mixture was stirred at 100° C. under argon for 2 h. The reaction was concentrated and dissolved in ACN (1 mL). The mixture was filtered and purified by preparative HPLC to give 6-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)isoindolin-1-one (7.5 mg, 51% yield) as white solid. ¹H NMR (400 MHz, CD₃OD) δ 8.38 (s, 1H), 8.28 (d, J=8.0 Hz, 1H), 7.81 (d, J=8.0 Hz, 1H), 7.77 (d, J=5.2 Hz, 1H), 7.69 (d, J=5.6 Hz, 1H), 6.81-6.88 (m, 2H), 6.70 (t, J=9.2 Hz, 1H), 6.20-6.35 (m, 2H), 5.82 (d, J=10.8 Hz, 1H), 5.55-5.75 (m, 1H), 4.58-4.63 (m, 3H), 4.35-4.47 (m, 1H), 4.16 (d, J=12.8 Hz, 1H), 3.96-4.12 (m, 3H), 3.65-3.86 (m, 1H) 3.43 (t, J=4.8 Hz, 2H), 3.03 (s, 3H), 1.35-1.58 (s, 3H). LCMS ESI (+) m/z 642.2 (M+H).

### Synthetic Example 74: Synthesis of 6-((S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl)isoindolin-1-one (Compound 852)

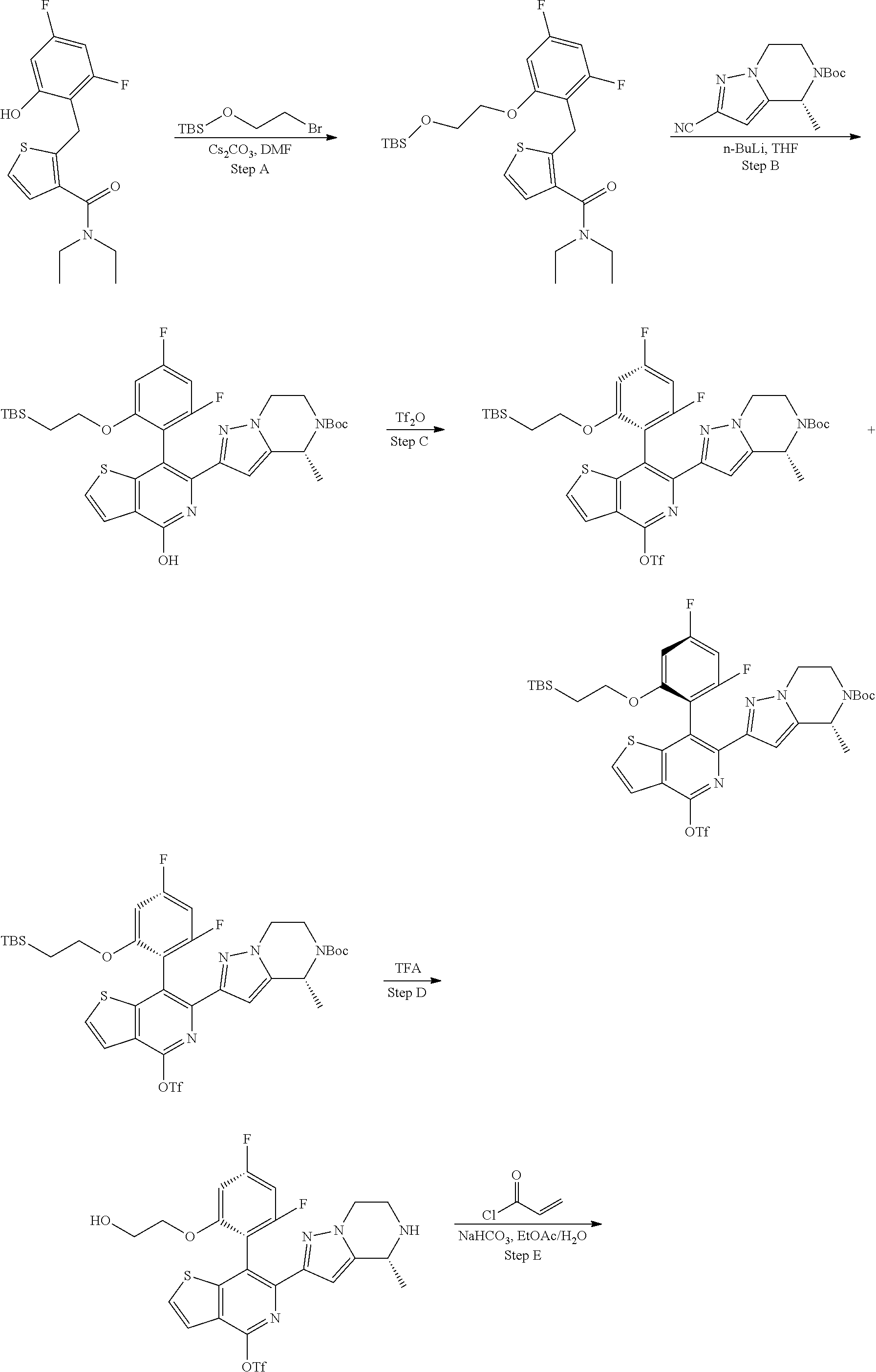

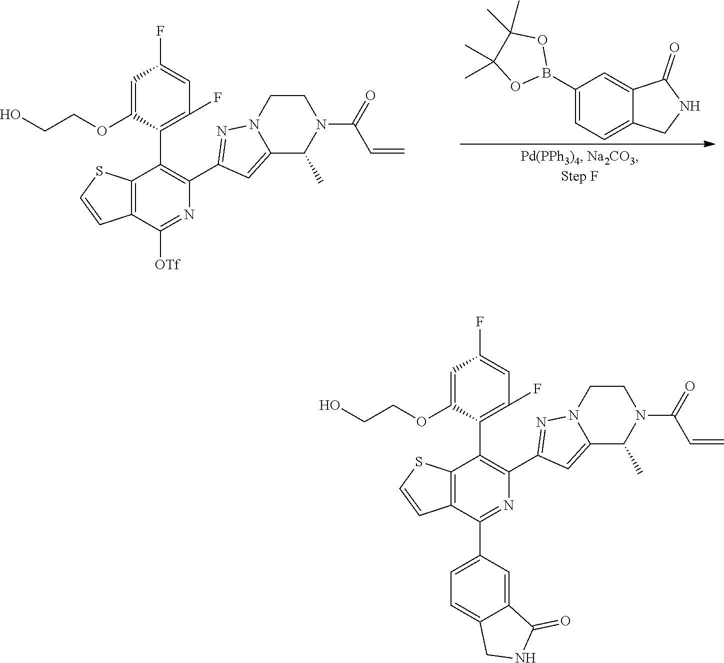

Step A: Preparation of 2-[[2-[2-[tert-butyl(dimethyl)silyl]oxyethoxy]-4,6-difluoro-phenyl]methyl]-N,N-diethyl-thiophene-3-carboxamide: To a solution of 2-[(2,4-difluoro-6-hydroxy-phenyl)methyl]—N,N-diethyl-thiophene-3-carboxamide (13.0 g, 40.0 mmol) and Cs₂CO₃ (19.5 g, 59.9 mmol) in DMF (200 mL) was added (2-bromoethoxy)-tert-butyldimethylsilane (10.0 mL, 47.9 mmol). The mixture was stirred at 70° C. for 3 h. The mixture was poured into water and the product was extracted with EtOAc/petroleum ether (1/1). The organics were washed with brine, dried over anhydrous Na₂SO₄, concentrated and purified by column chromatography on silica (0% to 20% EtOAc in petroleum ether) to give 2-[[2-[2-[tert-butyl(dimethyl)silyl]oxyethoxy]-4,6-difluoro-phenyl]methyl]-N,N-diethyl-thiophene-3-carboxamide (16.5 g, 85% yield). LCMS ESI (+) m/z 484.3 (M+H).

Step B: Preparation of tert-butyl (4R)-2-[7-[2-[2-[tert-butyl(dimethyl)silyl]oxyethoxy]-4,6-difluoro-phenyl]-4-hydroxy-thieno[3,2-c]pyridin-6-yl]-4-methyl-6,7-dihydro-4H-pyrazolo[1,5-a]pyrazine-5-carboxylate: To a solution of 2-[[2-[2-[tert-butyl(dimethyl)silyl]oxyethoxy]-4,6-difluoro-phenyl]methyl]-N,N-diethyl-thiophene-3-carboxamide (7.80 g, 16.1 mmol) in THF (100 mL) was added n-BuLi (2.5 M in hexanes, 7.00 mL, 17.6 mmol) dropwise under Ar at −60° C. The mixture was stirred at −60° C. for 0.5 h under Ar. Tert-butyl (4R)-2-cyano-4-methyl-6,7-dihydro-4H-pyrazolo[1,5-a]pyrazine-5-carboxylate (4.23 g, 16.1 mmol) in THF (20 mL) was added at −60° C. The reaction was allowed to warm to rt, and the resulting mixture was stirred at rt for additional 2 h. The mixture was poured into water. 1 N aqueous HCl was added to adjust the pH to 5. The mixture was concentrated. The residue was partitioned between EtOAc and water. The organic phase was dried over Na₂SO₄ and concentrated. The residue was purified by column chromatography on silica gel (petroleum ether/ethyl acetate=1/3) to give tert-butyl (4R)-2-[7-[2-[2-[tert-butyl(dimethyl)silyl]oxyethoxy]-4,6-difluoro-phenyl]-4-hydroxy-thieno[3,2-c]pyridin-6-yl]-4-methyl-6,7-dihydro-4H-pyrazolo[1,5-a]pyrazine-5-carboxylate (7.90 g, 66% yield). LCMS ESI (+) m/z 673.3 (M+H).

Step C: Preparation of tert-butyl (R)-2-((S)-7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate: To a solution of tert-butyl (4R)-2-[7-[2-[2-[tert-butyl(dimethyl)silyl]oxyethoxy]-4,6-difluoro-phenyl]-4-hydroxy-thieno[3,2-c]pyridin-6-yl]-4-methyl-6,7-dihydro-4H-pyrazolo[1,5-a]pyrazine-5-carboxylate (7.90 g, 10.6 mmol) and pyridine (2.60 mL, 31.7 mmol) in DCM (50 mL) was added trifluoromethanesulfonic anhydride (3.60 mL, 21.1 mmol). The mixture was stirred at rt for 1 h. The mixture was poured into water and the product was extracted with DCM. The organics were washed with saturated brine solution, dried over anhydrous Na₂SO₄, concentrated and purified by column chromatography on silica gel (EtOAc/petroleum ether: 1/15) to give the desired diastereomer tert-butyl (R)-2-((S)-7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (3.10 g, 36% yield) as the more polar product.

Step D: Preparation of (S)-7-(2,4-difluoro-6-(2-hydroxyethoxy)phenyl)-6-((R)-4-methyl-4,5,6,7-tetrahydropyrazolo[1,5-a]pyrazin-2-yl)thieno[3,2-c]pyridin-4-yl trifluoromethanesulfonate: To a solution of tert-butyl (R)-2-((S)-7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (505 mg, 0.627 mmol) in DCM (4 mL) was added trifluoroacetic acid (2.0 mL). The mixture was stirred at rt for 1 h. The solvent was concentrated under reduced pressure to afford (S)-7-(2,4-difluoro-6-(2-hydroxyethoxy)phenyl)-6-((R)-4-methyl-4,5,6,7-tetrahydropyrazolo[1,5-a]pyrazin-2-yl)thieno[3,2-c]pyridin-4-yl trifluoromethanesulfonate (530 mg) which was used in the next step without further purification.

Step E: Preparation (S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate: To a solution of [7-[2,4-difluoro-6-(2-hydroxyethoxy)phenyl]-6-[(4R)-4-methyl-4,5,6,7-tetrahydropyrazolo[1,5-a]pyrazin-2-yl]thieno[3,2-c]pyridin-4-yl]trifluoromethanesulfonate (530 mg, 0,610 mmol) in ethyl acetate (3 mL) and water (3 mL) was added sodium carbonate (256 mg, 3.05 mmol). The reaction mixture was stirred at 0° C. for 10 mins. A solution of acryloyl chloride (62 mg, 0.69 mmol) in DCM (2 mL) was added to the mixture and stirred at 0° C. for additional 30 min. The reaction was diluted with water and extracted with EtOAc. The organics were then dried (Na₂SO₄) and filtered before concentration to dryness to give crude (S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate (452 mg), which was used in the next step directly. LCMS ESI (+) m/z 645.0 (M+H).

Step F: Preparation of 6-((S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl)isoindolin-1-one: To a stirred solution of (S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate (24 mg, 0.037 mmol) in 1,4-dioxane (1 mL) and water (0.1 mL) were added Na₂CO₃ (12 mg, 0.11 mmol) and Pd(PPh₃)₄(4.3 mg, 0.0037 mmol). The mixture was stirred at 100° C. for 2 h under Argon. The reaction mixture was diluted with water and extracted with ethyl acetate. The organic layers were washed with brine, dried over Na₂SO₄, filtered and concentrated in vacuum. The residue was purified by preparative TLC (DCM:MeOH=20:1) followed by preparative HPLC to give 6-((S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl)isoindolin-1-one (7.4 mg, 31% yield) as a white solid. ¹H NMR (400 MHz, CD₃OD) δ 8.38 (s, 1H), 8.27 (dd, J=8.0, 1.6 Hz, 1H), 7.81 (d, J=8.0 Hz, 1H), 7.77 (d, J=5.6 Hz, 1H), 7.69 (d, J=5.6 Hz, 1H), 6.84 (dd, J=16.8, 10.6 Hz, 2H), 6.61-6.68 (m 1H), 6.29 (s, 2H), 5.81 (d, J=12.0 Hz, 1H), 5.43-5.68 (m, 1H), 4.60 (d, J=4.0 Hz, 3H), 4.12-4.22 (m, 1H), 3.91-4.08 (m, 3H), 3.50-3.64 (m, 2H), 1.40-1.56 (m, 3H). LCMS ESI (+) m/z 628.0 (M+H).

### Synthetic Example 75: Synthesis of 6-((S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl)-3,4-dihydroisoquinolin-1(2H)-one (Compound 876)

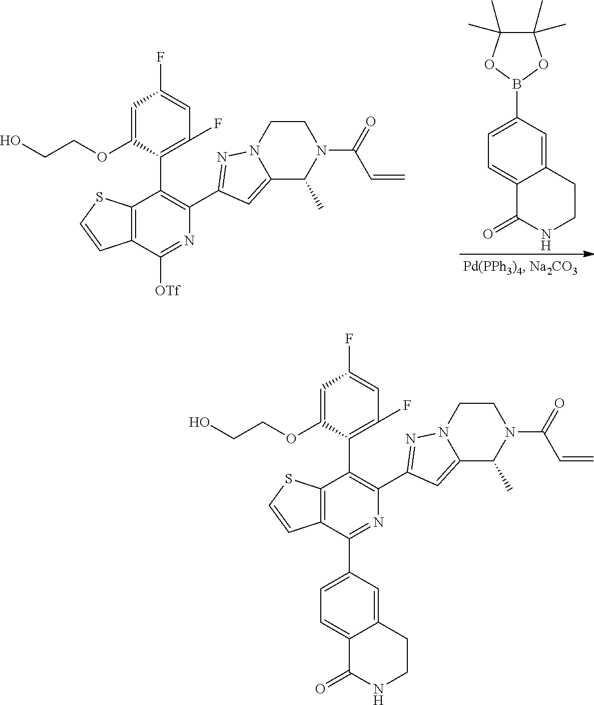

To a solution of (S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate (25 mg, 0.039 mmol), 6-(4,4,5,5-tetramethyl-1,3,2-dioxaborolan-2-yl)-3,4-dihydro-2H-isoquinolin-1-one (16 mg, 0.058 mmol) and sodium carbonate (2.0 mg, 0.019 mmol) in 1,4-dioxane (2 mL) and water (0.2 mL) was added tetrakis(triphenylphosphine)palladium(0) (4.5 mg, 0.0039 mmol). The mixture was stirred at 100° C. for 2 h under Ar. The mixture was poured into water and the product was extracted with EtOAc. The organic phase was washed with brine, dried over anhydrous Na₂SO₄, concentrated and purified by preparative HPLC to give 6-((S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl)-3,4-dihydroisoquinolin-1(2H)-one (9.3 mg, 37% yield) as a trifluoroacetic acid salt. ¹H NMR (400 MHz, CD₃OD) δ 8.22 (m, J=8.2 Hz, 1H), 8.02 (d, J=8.2 Hz, 1H), 7.93-7.98 (m, 2H), 7.77-7.80 (m, 1H), 6.95-6.98 (m, 1H), 6.75-6.87 (m, 2H), 6.27-6.31 (m, 1H), 6.11 (s, 1H), 5.81 (d, J=10.6 Hz, 1H), 5.40-5.71 (m, 1H), 4.44-4.49 (m, 1H), 4.27-4.31 (m, 1H), 4.01-4.08 (m, 3H), 3.71-3.85 (m, 1H), 3.55-3.63 (m, 4H), 3.15-3.19 (m, 2H), 1.26-1.50 (m, 3H). LCMS ESI (+) m/z 642.0 (M+H).

### Synthetic Example 76: Synthesis of 1-((R)-2-((S)-7-(2,4-difluoro-6-(2-hydroxyethoxy)phenyl)-4-(1-methyl-1H-indazol-5-yl)thieno[3,2-c]pyridin-6-yl)-4-methyl-6,7-dihydropyrazolo[1,5-a]pyrazin-5(4H)-yl)prop-2-en-1-one (Compound 908)

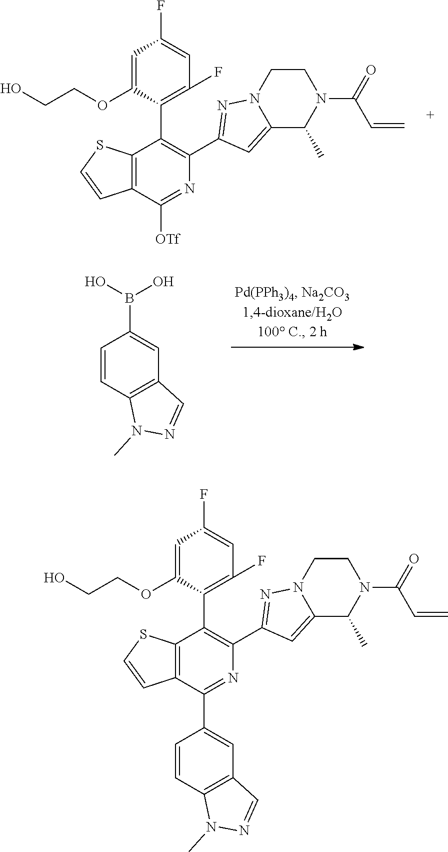

A mixture of (S)-6-((R)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate (30 mg, 0.047 mmol), (1-methylindazol-5-yl)boronic acid (16 mg, 0.093 mmol), Pd(PPh₃)₄(8.1 mg, 0.0070 mmol) and Na₂CO₃ (15 mg, 0.14 mmol) in 1,4-dioxane (2 mL)/water (0.2 mL) was stirred at 100° C. for 2 h under Ar. The mixture was filtered. The filtrate was concentrated and purified by preparative HPLC to give 1-((R)-2-((S)-7-(2,4-difluoro-6-(2-hydroxyethoxy)phenyl)-4-(1-methyl-1H-indazol-5-yl)thieno[3,2-c]pyridin-6-yl)-4-methyl-6,7-dihydropyrazolo[1,5-a]pyrazin-5(4H)-yl)prop-2-en-1-one (5.2 mg, 17% yield) as a white solid, ¹H NMR (400 MHz, CD₃OD) δ 8.36 (s, 1H), 8.16 (s, 1H), 8.07 (d, J=9.6 Hz, 1H), 7.59-7.82 (m, 3H), 6.75-6.97 (m, 2H), 6.68 (td, J=9.2, 2.4 Hz, 1H), 6.13-6.37 (m, 2H), 5.81 (d, J=10.6 Hz, 1H), 5.41-5.76 (m, 1H), 3.91-4.37 (m, 6H), 3.48-3.81 (m, 3H), 1.31-1.60 (m, 3H). LCMS ESI (+) m/z 626.8 (M+H).

### Synthetic Example 77: Synthesis of 1-((R)-2-((S)-7-(2,4-difluoro-6-(2-methoxyethoxy)phenyl)-4-(2-(4-methylpiperazin-1-yl)pyrimidin-5-yl)thieno[3,2-c]pyridin-6-yl)-4-methyl-6,7-dihydropyrazolo[1,5-a]pyrazin-5(4H)-yl)prop-2-en-1-one (Compound 951)

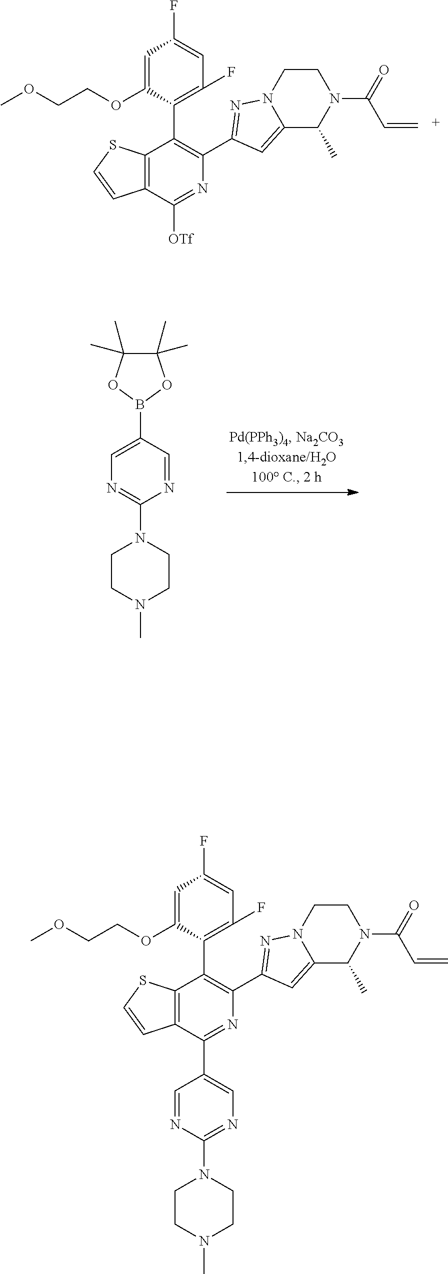

To a solution of (S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate (26 mg, 0.039 mmol), 2-(4-methylpiperazin-1-yl)-5-(4,4,5,5-tetramethyl-1,3,2-dioxaborolan-2-yl)pyrimidine (18 mg, 0.058 mmol) and sodium carbonate (2.0 mg, 0.019 mmol) in 1,4-dioxane (2 mL) and water (0.2 mL) was added tetrakis(triphenylphosphine)palladium(0) (4.5 mg, 0.0039 mmol). The mixture was stirred at 100° C. for 2 h under Ar. The mixture was concentrated and purified by preparative HPLC to give 1-((R)-2-((S)-7-(2,4-difluoro-6-(2-methoxyethoxy)phenyl)-4-(2-(4-methylpiperazin-1-yl)pyrimidin-5-yl)thieno[3,2-c]pyridin-6-yl)-4-methyl-6,7-dihydropyrazolo[1,5-a]pyrazin-5(4H)-yl)prop-2-en-1-one (12 mg, 43% yield) as a bis trifluoroacetic acid salt. ¹H NMR (400 MHz, CD₃OD) δ 9.07 (s, 2H), 7.86 (d, J=4.6 Hz, 1H), 7.73 (d, J=4.6 Hz, 1H), 6.81-6.92 (m, 2H), 6.70-6.75 (m, 1H), 6.27-6.31 (m, 1H), 6.11 (s, 1H), 5.84 (d, J=10.6 Hz, 1H), 5.45-5.75 (m, 1H), 5.09-5.19 (m, 2H), 4.35-4.49 (m, 1H), 4.22-4.26 (m, 1H), 4.02-4.12 (m, 3H), 3.55-3.81 (m, 3H), 3.42 (t, J=4.4 Hz, 4H), 3.15-3.25 (m, 2H), 3.05 (s, 3H), 2.99 (s, 3H), 1.35-1.50 (m, 3H). LCMS ESI (+) m/z 687.2 (M+H).

### Synthetic Example 78: Synthesis of 1-((S)-2-((S)-7-(2,4-difluoro-6-(2-hydroxyethoxy)phenyl)-4-(6-(dimethylamino)pyridin-3-yl)thieno[3,2-c]pyridin-6-yl)-4-methyl-6,7-dihydropyrazolo[1,5-a]pyrazin-5(4H)-yl)prop-2-en-1-one (Compound 957)

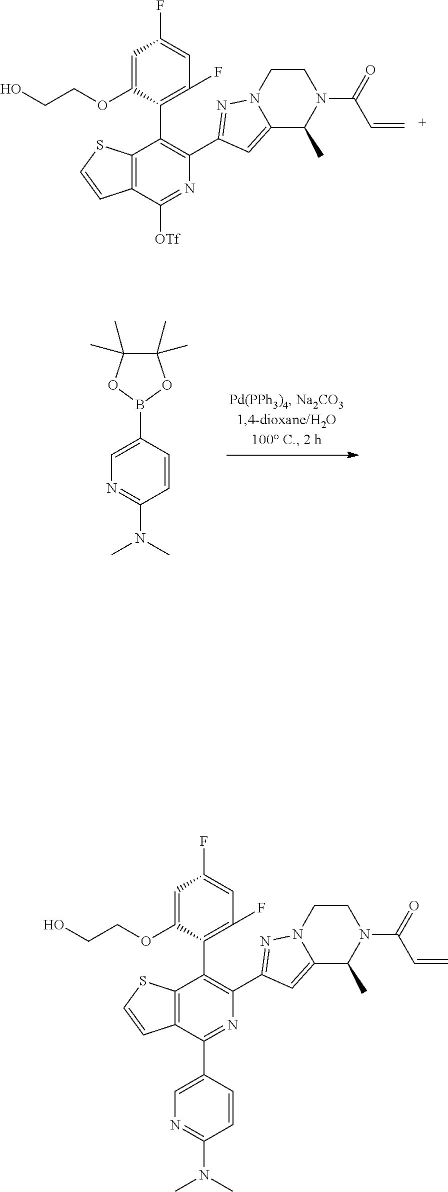

A suspension of (S)-6-((S)-5-acryloyl-4-methyl-4,5,6,7-tetrahydropyrazolo[1,5-a]pyrazin-2-yl)-7-(2,4-difluoro-6-(2-hydroxyethoxy)phenyl)thieno[3,2-c]pyridin-4-yl trifluoromethanesulfonate (23 mg, 0.036 mmol), N,N-dimethyl-5-(4,4,5,5-tetramethyl-1,3,2-dioxaborolan-2-yl)pyridin-2-amine (18 mg, 0.071 mmol), tetrakis(triphenylphosphine)palladium(0) (4.1 mg, 0.0036 mmol) and sodium carbonate (7.6 mg, 0.071 mmol) in 1,4-dioxane (1.2 mL) and water (0.3 mL) was stirred for 4 h at 100° C. under N₂ atmosphere. The mixture was diluted with water and the residue was taken up in EtOAc. The organics were washed with saturated brine solution, dried over Na₂SO₄ and concentrated to dryness. The crude was purified by preparative HPLC to give 1-((S)-2-((S)-7-(2,4-difluoro-6-(2-hydroxyethoxy)phenyl)-4-(6-(dimethylamino)pyridin-3-yl)thieno[3,2-c]pyridin-6-yl)-4-methyl-6,7-dihydropyrazolo[1,5-a]pyrazin-5(4H)-yl)prop-2-en-1-one (18 mg, 80% yield) as a trifluoroacetic acid salt. ¹H NMR (400 MHz, CD₃OD) δ 8.62 (dd, J=2.0, 9.2 Hz, 1H), 8.56 (d, J=2.0 Hz, 1H), 7.89 (d, J=5.6 Hz, 1H), 7.76 (d, J=5.6 Hz, 1H), 7.36-7.41 (m, 1H), 6.86-6.90 (m, 1H), 6.78-6.85 (m, 1H), 6.71-6.77 (m, 1H), 6.24-6.35 (m, 1H), 6.09-6.15 (m, 1H), 5.83 (dd, J=1.6, 10.8 Hz, 1H), 5.49-5.70 (m, 1H), 4.39-4.47 (m, 1H), 4.19-4.27 (m, 1H), 4.04-4.15 (m, 1H), 3.96-4.03 (m, 1H), 3.88-3.95 (m, 1H), 3.66-3.82 (m, 1H), 3.50-3.56 (m, 2H), 3.39 (s, 6H), 1.39-1.51 (m, 3H). LCMS ESI (+) m/z 617.2 (M+H).

### Synthetic Example 79: Synthesis of 1-[7-[2,4-difluoro-6-(2-methoxyethoxy)phenyl]-6-[(6R)-6-methyl-5-prop-2-enoyl-6,7-dihydro-4H-pyrazolo[1,5-a]pyrazin-2-1]thieno[3,2-c]pyridin-4-yl]pyridin-2-one (Compound 964)

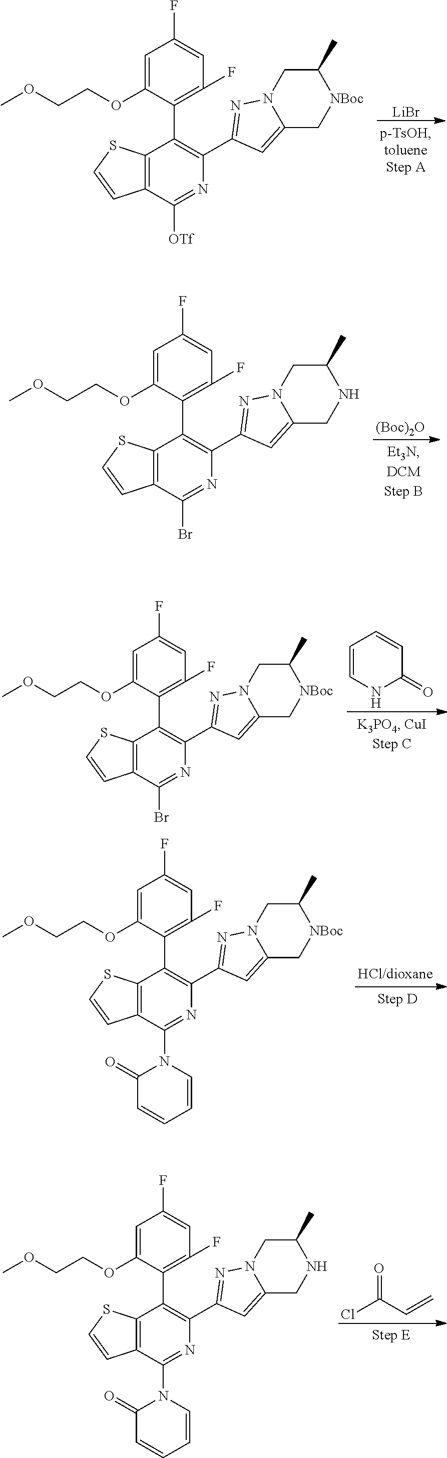

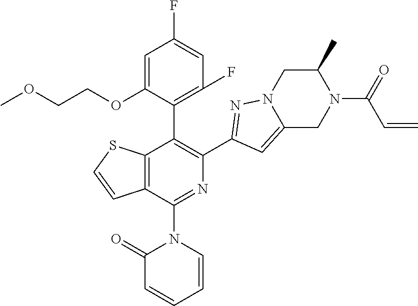 [SMILES: C=CC(=O)N1Cc2cc(-c3nc(-n4ccccc4=O)c4ccsc4c3-c3c(F)cc(F)cc3OCCOC)nn2C[C@H]1C]

Step A: Preparation of 4-bromo-7-[2,4-difluoro-6-(2-methoxyethoxy)phenyl]-6-[(6R)-6-methyl-4,5,6,7-tetrahydro pyrazolo[1,5-a]pyrazin-2-yl]thieno[3,2-c]pyridine: A mixture of tert-butyl (6R)-2-[7-[2,4-difluoro-6-(2-methoxyethoxy)phenyl]-4-(trifluoromethylsulfonyloxy)thieno[3,2-c]pyridin-6-yl]-6-methyl-6,7-dihydro-4H-yrazolo[1,5-a]pyrazine-5-carboxylate (800 mg, 1.14 mmol), LiBr (493 mg, 5.68 mmol) and p-toluenesulfonic acid monohydrate (235 mg, 1.36 mmol) was stirred in toluene (15 mL) at 50° C. overnight. The reaction mixture was concentrated and purified by flash column chromatography eluting with 60% EtOAc in hexanes to give 4-bromo-7-[2,4-difluoro-6-(2-methoxyethoxy)phenyl]-6-[(6R)-6-methyl-4,5,6,7-tetrahydropyrazolo[1,5-a]pyrazin-2-yl]thieno[3,2-c]pyridine (600 mg, 99% yield). LCMS ESI (+) m/z 535.1 (M+H).

Step B: Preparation of tert-butyl (6R)-2-[4-bromo-7-[2,4-difluoro-6-(2-methoxyethoxy)phenyl]thieno[3,2-c]pyridin-6-yl]-6-methyl-6,7-dihydro-4H-pyrazolo[1,5-a]pyrazine-5-carboxylate: To a solution of 4-bromo-7-[2,4-difluoro-6-(2-methoxyethoxy)phenyl]-6-[(6R)-6-methyl-4,5,6,7-tetrahydropyrazolo[1,5-a]pyrazin-2-yl]thieno[3,2-c]pyridine (600 mg, 1.12 mmol) and Et₃N (226 mg, 2.24 mmol) in DCM (10 mL) was added (Boc)₂O (318 mg, 1.46 mmol) and the mixture was stirred at rt overnight. The reaction was concentrated to dryness and the residue was then purified by flash column chromatography on silica gel eluting with 25% EtOAc in hexanes to give tert-butyl (6R)-2-[4-bromo-7-[2,4-difluoro-6-(2-methoxyethoxy)phenyl]thieno[3,2-c]pyridin-6-yl]-6-methyl-6,7-dihydro-4H-yrazolo[1,5-a]pyrazine-5-carboxylate (550 mg, 77% yield). LCMS ESI (+) m/z 634.7 (M+H).

Step C: Preparation of tert-butyl (6R)-2-[7-[2,4-difluoro-6-(2-methoxyethoxy)phenyl]-4-(2-oxo-1-pyridyl)thieno[3,2-c]pyridin-6-yl]-6-methyl-6,7-dihydro-4H-pyrazolo[1,5-a]pyrazine-5-carboxylate: A mixture of tert-butyl (6R)-2-[4-bromo-7-[2,4-difluoro-6-(2-methoxyethoxy)phenyl]thieno[3,2-c]pyridin-6-yl]-6-methyl-6,7-dihydro-4H-pyrazolo[1,5-a]pyrazine-5-carboxylate (200 mg, 0.315 mmol), pyridin-2-one (36 mg, 0.38 mmol), CuI (6.0 mg, 0.032 mmol), 1,2-dimethylethylenediamine (5.5 mg, 0.063 mmol) and K₃PO₄ (134 mg, 0.629 mmol) in toluene (8 mL) was stirred at 110° C. overnight under nitrogen. The reaction was concentrated to dryness. The crude was purified by flash column chromatography eluting with 5% MeOH in DCM to give tert-butyl (6R)-2-[7-[2,4-difluoro-6-(2-methoxyethoxy)phenyl]-4-(2-oxo-1-pyridyl)thieno[3,2-c]pyridin-6-yl]-6-methyl-6,7-dihydro-4H-pyrazolo[1,5-a]pyrazine-5-carboxylate (70 mg, 34% yield) as a pale brown solid. LCMS ESI (+) m/z 649.8 (M+H).

Step D: Preparation of 1-(7-(2,4-difluoro-6-(2-methoxyethoxy)phenyl)-6-((R)-6-methyl-4,5,6,7-tetrahydropyrazolo[1,5-a]pyrazin-2-yl)thieno[3,2-c]pyridin-4-yl)pyridin-2(1H)-one: To a solution of tert-butyl (6R)-2-[7-[2,4-difluoro-6-(2-methoxyethoxy)phenyl]-4-(2-oxo-1-pyridyl)thieno[3,2-c]pyridin-6-yl]-6-methyl-6,7-dihydro-4H-pyrazolo[1,5-a]pyrazine-5-carboxylate (70 mg, 0.11 mmol) in DCM (3 mL) was added 4 N HCl in dioxane (2.0 mL, 8.0 mmol). The mixture was stirred at 25° C. overnight and then concentrated to give crude (60 mg) 1-(7-(2,4-difluoro-6-(2-methoxyethoxy)phenyl)-6-((R)-6-methyl-4,5,6,7-tetrahydropyrazolo[1,5-a]pyrazin-2-yl)thieno[3,2-c]pyridin-4-yl)pyridin-2(1H)-one, which was used directly in the next step without further purification. LCMS ESI (+) m/z 549.8 (M+H).

Step E: Preparation of 1-[7-[2,4-difluoro-6-(2-methoxyethoxy)phenyl]-6-[(6R)-6-methyl-5-prop-2-enoyl-6,7-dihydro-4H-pyrazolo[1,5-a]pyrazin-2-1]thieno[3,2-c]pyridin-4-yl]pyridin-2-one: To a solution of 1-[7-[2,4-difluoro-6-(2-methoxyethoxy)phenyl]-6-[(6R)-6-methyl-4,5,6,7-tetrahydropyrazolo[1,5-a]pyrazin-2-yl]thieno[3,2-c]pyridin-4-yl]pyridin-2-one (60 mg, 0.11 mmol) and DIEA (0.097 mL, 0.55 mmol) in DCM (4 mL) was added acryloyl chloride (0.018 mL, 0.22 mmol) at −15° C. The mixture was stirred at −15° C. for 40 min. The reaction was concentrated to dryness and the crude was then purified by flash column chromatography on silica gel eluting with 7% MeOH in DCM. The desired fractions were concentrated and then purified by preparative TLC (MeOH/DCM=1:12) to give 1-[7-[2,4-difluoro-6-(2-methoxyethoxy)phenyl]-6-[(6R)-6-methyl-5-prop-2-enoyl-6,7-dihydro-4H-pyrazolo[1,5-a]pyrazin-2-1]thieno[3,2-c]pyridin-4-yl]pyridin-2-one (14 mg, 22% yield) as a white solid. ¹H NMR (400 MHz, CD₃OD) δ 7.93 (d, J=6.8 Hz, 1H), 7.81-7.73 (m, 2H), 7.27 (d, J=5.6 Hz, 1H), 6.96-6.80 (m, 2H), 6.79-6.70 (m, 2H), 6.61 (t, J=6.8 Hz, 1H), 6.33-6.22 (m, 2H), 5.82 (dd, J=10.6, 1.7 Hz, 1H), 5.14 (d, J=17.5 Hz, 1H), 4.74 (m, 1H), 4.47-4.15 (m, 2H), 4.12-3.97 (m, 3H), 3.43 (dd, J=9.0, 4.5 Hz, 2H), 3.05 (d, J=1.4 Hz, 3H), 1.18 (s, 3H). LCMS ESI (+) m/z 603.8 (M+H).

### Synthetic Example 80: Synthesis of 2-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-N-methyl-2-azaspiro[3.3]heptane-6-carboxamide (Compound 1012)

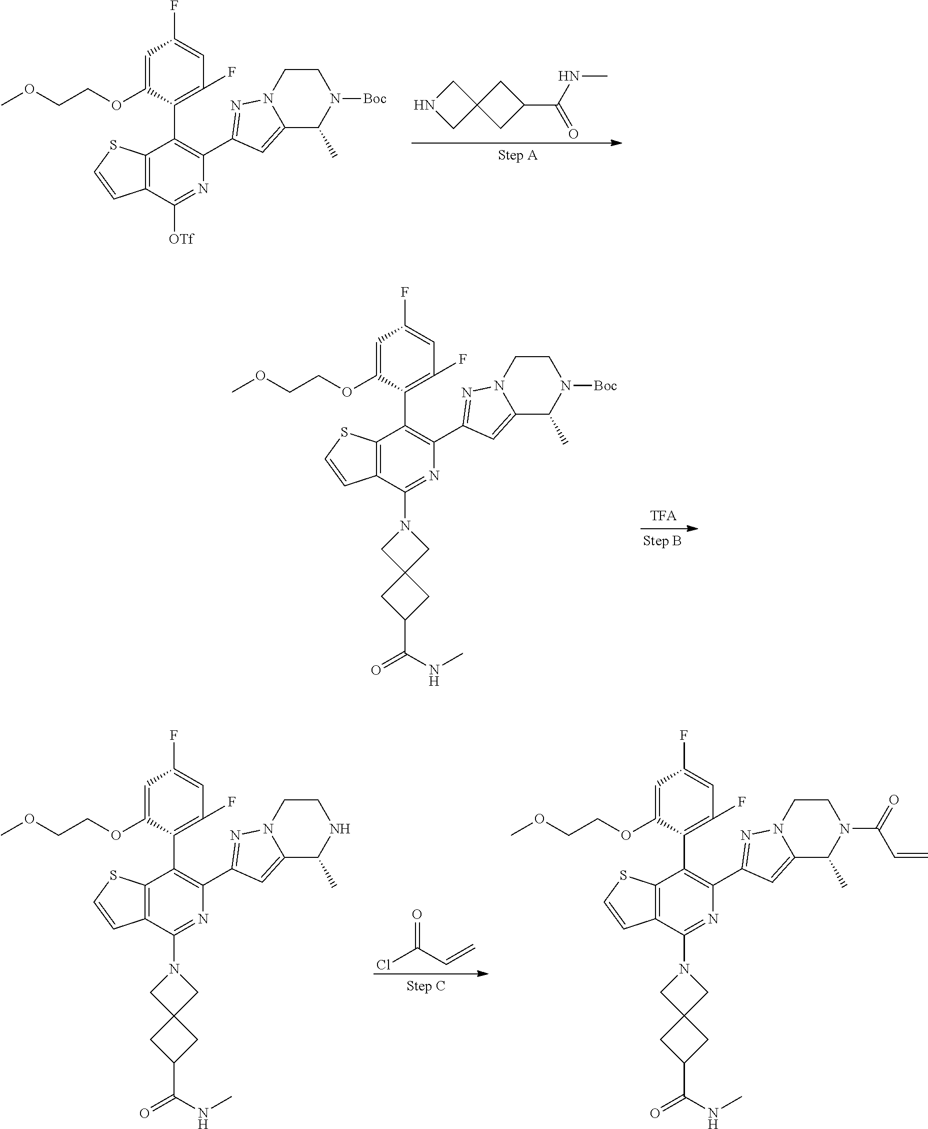

Step A: Preparation of tert-butyl (R)-2-((S)-7-(2,4-difluoro-6-(2-methoxyethoxy)phenyl)-4-(6-(methylcarbamoyl)-2-azaspiro[3.3]heptan-2-yl)thieno[3,2-c]pyridin-6-yl)-4-methyl-6,7-dihydropyrazolo[1,5-a]pyrazine-5(4H)-carboxylate: To a solution of tert-butyl (R)-2-((S)-7-(2,4-difluoro-6-(2-methoxyethoxy)phenyl)-4-(((trifluoromethyl)sulfonyl)oxy)thieno[3,2-c]pyridin-6-yl)-4-methyl-6,7-dihydropyrazolo[1,5-a]pyrazine-5(4H)-carboxylate (30 mg, 0.043 mmol) in DMSO (2 mL) were added N-methyl-2-azaspiro[3.3]heptane-6-carboxamide (33 mg, 0.21 mmol) and triethylamine (0.12 mL, 0.85 mmol). The mixture was stirred at 100° C. for 10 h. The solution was poured into water and the product was extracted with EtOAc. The combined organic layers were washed with brine, dried and concentrated. The residue was purified by preparative TLC (EtOAc/petroleum ether: 1/1) to give tert-butyl (R)-2-((S)-7-(2,4-difluoro-6-(2-methoxyethoxy)phenyl)-4-(6-(methylcarbamoyl)-2-azaspiro[3.3]heptan-2-yl)thieno[3,2-c]pyridin-6-yl)-4-methyl-6,7-dihydropyrazolo[1,5-a]pyrazine-5(4H)-carboxylate (29 mg, 96% yield).

Step B: Preparation of 2-((S)-7-(2,4-difluoro-6-(2-methoxyethoxy)phenyl)-6-((R)-4-methyl-4,5,6,7-tetrahydropyrazolo[1,5-a]pyrazin-2-yl)thieno[3,2-c]pyridin-4-yl)-N-methyl-2-azaspiro[3.3]heptane-6-carboxamide: To a solution of tert-butyl (R)-2-((S)-7-(2,4-difluoro-6-(2-methoxyethoxy)phenyl)-4-(6-(methylcarbamoyl)-2-azaspiro[3.3]heptan-2-yl)thieno[3,2-c]pyridin-6-yl)-4-methyl-6,7-dihydropyrazolo[1,5-a]pyrazine-5(4H)-carboxylate (29 mg, 0.041 mmol) in DCM (3 mL) was added trifluoroacetic acid (1.0 mL). The mixture was stirred at 25° C. for 1 h. The solution was concentrated to dryness to give crude 2-((S)-7-(2,4-difluoro-6-(2-methoxyethoxy)phenyl)-6-((R)-4-methyl-4,5,6,7-tetrahydropyrazolo[1,5-a]pyrazin-2-yl)thieno[3,2-c]pyridin-4-yl)-N-methyl-2-azaspiro[3.3]heptane-6-carboxamide (38 mg) which was used in the next step without further purification.

Step C: Preparation of 2-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-N-methyl-2-azaspiro[3.3]heptane-6-carboxamide: To a solution of 2-((S)-7-(2,4-difluoro-6-(2-methoxyethoxy)phenyl)-6-((R)-4-methyl-4,5,6,7-tetrahydropyrazolo[1,5-a]pyrazin-2-yl)thieno[3,2-c]pyridin-4-yl)-N-methyl-2-azaspiro[3.3]heptane-6-carboxamide (38 mg) in ethyl acetate (2 mL)/water (2 mL) was cooled to 0° C. Na₂CO₃ was added to adjust the pH to 9. Acryloyl chloride (0.0050 mL, 0.062 mmol) in DCM (1 mL) was added at 0° C. The mixture was stirred at 0° C. for 1 hour. The organics were separated, dried and concentrated. The residue was purified by preparative HPLC to give 2-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-N-methyl-2-azaspiro[3.3]heptane-6-carboxamide (7.8 mg, 28% yield) as a trifluoracetic acid salt. ¹H NMR (400 MHz, CD₃OD) δ 7.87 (d, J=5.6 Hz, 1H), 7.81 (d, J=5.6 Hz, 1H), 6.97 (d, J=10.8 Hz, 1H), 6.79 (t, J=9.1 Hz, 2H), 6.29 (d, J=16.4 Hz, 1H), 5.83 (dd, J=1.7, 10.6 Hz, 1H), 5.71 (s, 1H), 5.41-5.70 (m, 1H), 4.79 (d, J=11.4 Hz, 4H), 4.37 (dd, J=2.9, 12.9 Hz, 1H), 4.09-4.18 (m, 3H), 3.77 (s, 1H), 3.40-3.45 (m, 2H), 3.08 (s, 3H), 2.98-3.04 (m, 1H), 2.74 (s, 3H), 2.51-2.65 (m, 4H), 1.36-1.49 (m, 3H). LCMS ESI (+) m/z 663.3 (M+H).

### Synthetic Example 82: Synthesis of 3-((S)-6-((R)-5-acryloyl-6-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-N-methylbicyclo[1.1.1]pentane-1-carboxamide (Compound 1071)

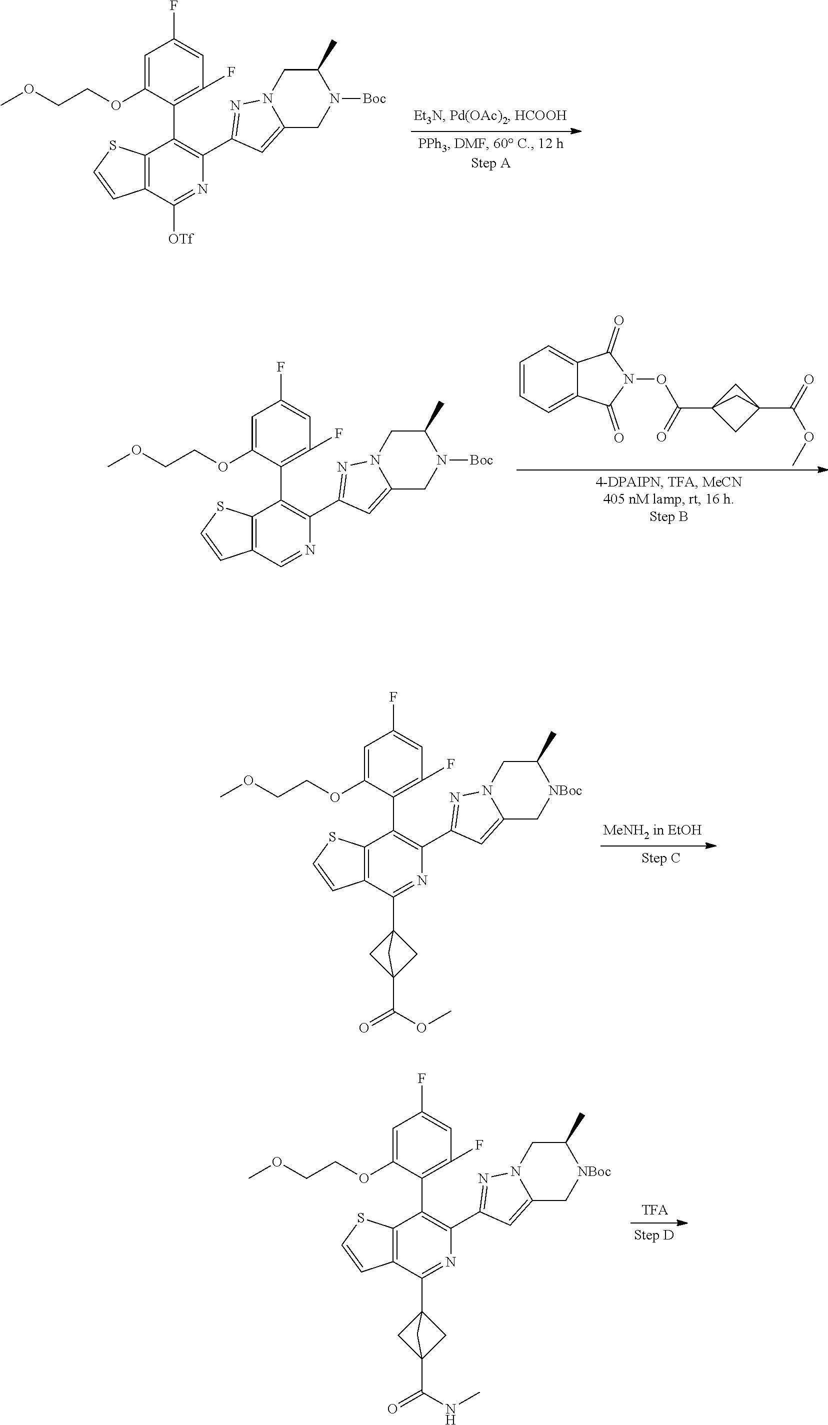 [SMILES: COCCOc1cc(F)cc(F)c1-c1c(-c2cc3n(n2)C[C@@H](C)N(C(=O)OC(C)(C)C)C3)nc(OS(=O)(=O)C(F)(F)F)c2ccsc12 ; COCCOc1cc(F)cc(F)c1-c1c(-c2cc3n(n2)C[C@@H](C)N(C(=O)OC(C)(C)C)C3)ncc2ccsc12 ; COC(=O)C12CC(C(=O)ON3C(=O)c4ccccc4C3=O)(C1)C2 ; COCCOc1cc(F)cc(F)c1-c1c(-c2cc3n(n2)C[C@@H](C)N(C(=O)OC(C)(C)C)C3)nc(C23CC(C(=O)OC)(C2)C3)c2ccsc12 ; CNC(=O)C12CC(c3nc(-c4cc5n(n4)C[C@@H](C)N(C(=O)OC(C)(C)C)C5)c(-c4c(F)cc(F)cc4OCCOC)c4sccc34)(C1)C2]

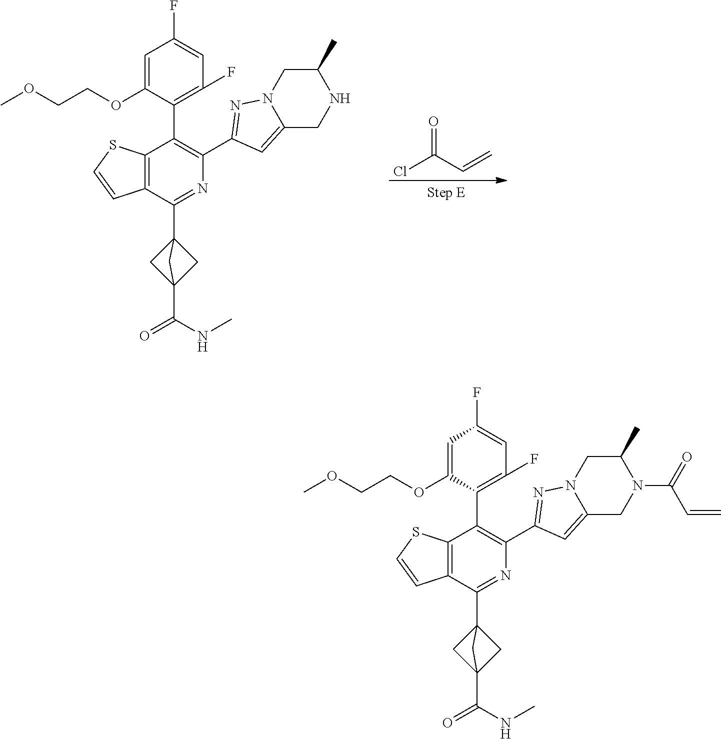

Step A: Preparation of tert-butyl (6R)-2-[7-[2,4-difluoro-6-(2-methoxyethoxy)phenyl]thieno[3,2-c]pyridin-6-yl]-6-methyl-6,7-dihydro-4H-pyrazolo[1,5-a]pyrazine-5-carboxylate: A mixture of tert-butyl (6R)-2-[7-[2,4-difluoro-6-(2-methoxyethoxy)phenyl]-4-(trifluoromethylsulfonyloxy)thieno[3,2-c]pyridin-6-yl]-6-methyl-6,7-dihydro-4H-pyrazolo[1,5-a]pyrazine-5-carboxylate (200 mg, 0.284 mmol), Et₃N (86 mg, 0.85 mmol), formic acid (0.033 mL, 0.85 mmol), PPh₃ (7.4 mg, 0.028 mmol) and Pd(OAc)₂ (6.4 mg, 0.028 mmol) in DMF (6 mL) was stirred at 60° C. under N₂ for 12 h. Upon completion of the reaction, the mixture was diluted with water. The aqueous layer was separated and extracted with EtOAc. The organic layers were combined, dried over anhydrous Na₂SO₄ and concentrated under reduced pressure. The residue was purified by flash column chromatography on silica gel (EtOAc/petroleum ether=0 to 60%) to give tert-butyl (6R)-2-[7-[2,4-difluoro-6-(2-methoxyethoxy)phenyl]thieno[3,2-c]pyridin-6-yl]-6-methyl-6,7-dihydro-4H-pyrazolo[1,5-a]pyrazine-5-carboxylate (100 mg, 63% yield) as a yellow oil. LCMS ESI (+) m/z 557.0 (M+H).

Step B: Preparation of tert-butyl (6R)-2-[7-[2,4-difluoro-6-(2-methoxyethoxy)phenyl]-4-(3-methoxycarbonyl-1-bicyclo[1.1.1]pentanyl)thieno[3,2-c]pyridin-6-yl]-6-methyl-6,7-dihydro-4H-pyrazolo[1,5-a]pyrazine-5-carboxylate: A mixture of 1-(1,3-dioxoisoindolin-2-yl) 3-methyl bicyclo[1.1.1]pentane-1,3-dicarboxylate (60 mg, 0.19 mmol), tert-butyl (6R)-2-[7-[2,4-difluoro-6-(2-methoxyethoxy)phenyl]thieno[3,2-c]pyridin-6-yl]-6-methyl-6,7-dihydro-4H-pyrazolo[1,5-a]pyrazine-5-carboxylate (106 mg, 0.190 mmol), 1,3-dicyano-2,4,5,6-tetrakis(diphenylamino)-benzene (3.8 mg, 0.0048 mmol) and TFA (0.029 mL, 0.38 mmol) was stirred in a Heptochem reactor with a 405 nM lamp for 16 hours. Upon completion of the reaction, the reaction mixture was concentrated in vacuo. The mixture was diluted with water and basified by NaHCO₃. The mixture was extracted with EtOAc. The organic layers were combined, dried over MgSO₄ and concentrated under reduced pressure. The crude was purified by column chromatography on silica gel (MeOH/DCM=1/10) to give tert-butyl (6R)-2-[7-[2,4-difluoro-6-(2-methoxyethoxy)phenyl]-4-(3-methoxycarbonyl-1-bicyclo[1.1.1]pentanyl)thieno[3,2-c]pyridin-6-yl]-6-methyl-6,7-dihydro-4H-pyrazolo[1,5-a]pyrazine-5-carboxylate (70 mg, 49% yield). LCMS ESI (+) m/z 683.1 (M+H).

Step C: Preparation of tert-butyl (6R)-2-[7-[2,4-difluoro-6-(2-methoxyethoxy)phenyl]-4-[3-(methylcarbamoyl)-1-bicyclo[1.1.1]pentanyl]thieno[3,2-c]pyridin-6-yl]-6-methyl-6,7-dihydro-4H-pyrazolo[1,5-a]pyrazine-5-carboxylate: A mixture of tert-butyl (6R)-2-[7-[2,4-difluoro-6-(2-methoxyethoxy)phenyl]-4-(3-methoxycarbonyl-1-bicyclo[1.1.1]pentanyl)thieno[3,2-c]pyridin-6-yl]-6-methyl-6,7-dihydro-4H-pyrazolo[1,5-a]pyrazine-5-carboxylate (90 mg, 0.13 mmol) and MeNH₂ in ethanol (3.00 mL, 1.32 mmol) was stirred at 110° C. in sealed tube for 12 h. Upon completion of the reaction. The resulting mixture was concentrated to give crude product, which was purified by column chromatography on silica gel (MeOH/DCM=1/10) to give tert-butyl (6R)-2-[7-[2,4-difluoro-6-(2-methoxyethoxy)phenyl]-4-[3-(methylcarbamoyl)-1-bicyclo[1.1.1]pentanyl]thieno[3,2-c]pyridin-6-yl]-6-methyl-6,7-dihydro-4H-pyrazolo[1,5-a]pyrazine-5-carboxylate (80 mg, 86% yield). LCMS ESI (+) m/z 680.3 (M+H).

Step D: Preparation of 3-[7-[2,4-difluoro-6-(2-methoxyethoxy)phenyl]-6-[(6R)-6-methyl-4,5,6,7-tetrahydropyrazolo[1,5-a]pyrazin-2-yl]thieno[3,2-c]pyridin-4-yl]-N-methyl-bicyclo[1.1.1]pentane-1-carboxamide: A mixture of tert-butyl (6R)-2-[7-[2,4-difluoro-6-(2-methoxyethoxy)phenyl]-4-[3-(methylcarbamoyl)-1-bicyclo[1.1.1]pentanyl]thieno[3,2-c]pyridin-6-yl]-6-methyl-6,7-dihydro-4H-pyrazolo[1,5-a]pyrazine-5-carboxylate (70 mg, 0.10 mmol) and TFA (1 mL) in DCM (5 mL) was stirred at rt for 12 h. Upon completion of the reaction, the reaction mixture was concentrated in vacuo. The mixture was diluted with water and then made basic with NaHCO₃. The mixture was extracted with EtOAc. The organic layers were combined, dried over MgSO₄ and concentrated under reduced pressure to give 3-[7-[2,4-difluoro-6-(2-methoxyethoxy)phenyl]-6-[(6R)-6-methyl-4,5,6,7-tetrahydropyrazolo[1,5-a]pyrazin-2-yl]thieno[3,2-c]pyridin-4-yl]-N-methyl-bicyclo[0.1.1]pentane-1-carboxamide (70 mg, 100% yield). LCMS ESI (+) m/z 580.0 (M+H).

Step E: Preparation of 3-((S)-6-((R)-5-acryloyl-6-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-N-methylbicyclo[1.1.1]pentane-1-carboxamide: To a solution of 3-[7-[2,4-difluoro-6-(2-methoxyethoxy)phenyl]-6-[(6R)-6-methyl-4,5,6,7-tetrahydropyrazolo[1,5-a]pyrazin-2-yl]thieno[3,2-c]pyridin-4-yl]-N-methyl-bicyclo[1.1.1]pentane-1-carboxamide (70 mg, 0.12 mmol) and DIEA (24 mg, 0.24 mmol) in DCM (10 mL) was added acryloyl chloride (11 mg, 0.13 mmol) at 0° C. and the mixture were stirred at 25° C. for 2 h. Upon completion of the reaction, the mixture was partitioned between DCM and water. The organic layer was separated and dried (MgSO₄) before concentration to dryness. The crude was then purified by flash column chromatography on silica gel eluting with 100% EtOAc to give a mixture of two diastereomers, which was further purified by chiral SFC (CHIRALPAK® IC 250 mm×20 mm, 5 m; Mobile phase: CO₂-EtOH (0.2% diethyl amine)) to afford 3-((S)-6-((R)-5-acryloyl-6-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-N-methylbicyclo[1.1.1]pentane-1-carboxamide (4.3 mg, 5% yield). ¹H NMR (400 MHz. CD₃OD) S 7.70 (d, J=5.6 Hz, 1H), 7.61 (d, J=5.6 Hz, 1H), 6.71 (d, J=10.8 Hz, 2H), 6.55 (td, J=9.2, 2.2 Hz, 1H), 6.22-6.09 (m, 2H), 5.71 (dd, J=10.6, 1.8 Hz, 1H), 5.02 (s, 1H), 4.75-4.47 (m, 1H), 4.30-4.04 (m, 2H), 4.00-3.84 (m, 3H), 3.29 (t, J=4.6 Hz, 2H), 2.91 (s, 3H), 2.69 (s, 3H), 2.53 (s, 6H), 1.10 (s, 3H). LCMS ESI (+) m/z 634.2 (M+H).

### Synthetic Example 83: Synthesis of 8-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-4-methyl-1,3,4,5-tetrahydro-2H-benzo[e][1,4]diazepin-2-one (Compound 1132)

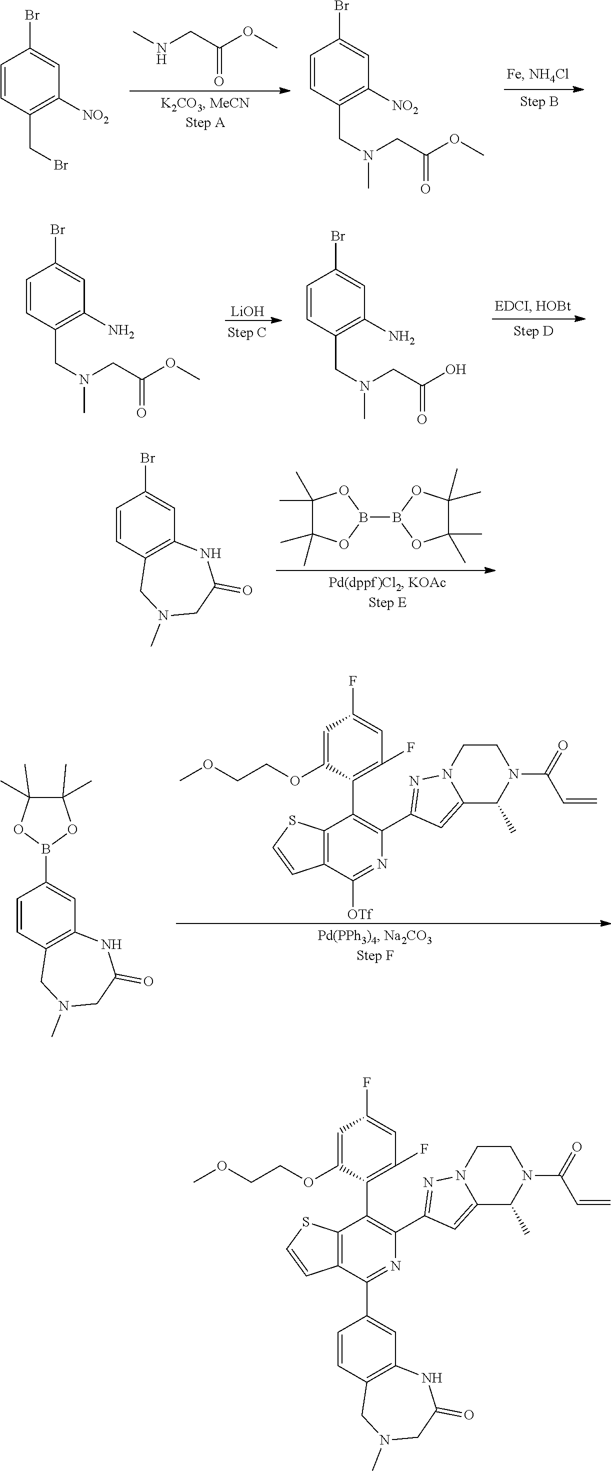

Step A: Preparation of methyl 2-[(4-bromo-2-nitro-phenyl)methyl-methyl-amino]acetate: To a stirred suspension of 4-bromo-1-(bromomethyl)-2-nitro-benzene (2.00 g, 6.78 mmol), K₂CO₃ (1.01 g, 10.2 mmol) in MeCN (20 mL) was added methyl 2-(methylamino)acetate (0.730 g, 7.12 mmol). The mixture was stirred at 60° C. for 16 h under Argon. To the reaction was added water. The mixture was extracted with EtOAc. The combined organic layers were washed with saturated brine solution, dried over Na₂SO₄, and concentrated in vacuum. The residue was purified by column chromatography on silica gel (petroleum ether:EtOAc=20:1) to afford methyl 2-[(4-bromo-2-nitro-phenyl)methyl-methyl-amino]acetate (1.50 g, 70% yield).

Step B: Preparation of methyl 2-[(2-amino-4-bromo-phenyl)methyl-methyl-amino]acetate: To a stirred solution of methyl 2-[(4-bromo-2-nitro-phenyl)methyl-methyl-amino]acetate (1.50 g, 4.73 mmol) in methanol (40 mL) was added aqueous NH₄Cl (4.7 M, 10 ml) and iron powder (1.32 g, 23.6 mmol). The reaction was stirred at 70° C. for 4 h under Argon. The reaction was filtered and concentrated to remove MeOH and then diluted with water (100 mL). The mixture was extracted with EtOAc. The combined organic layers were washed with saturated brine solution, dried over Na₂SO₄ and concentrated to give methyl 2-[(2-amino-4-bromo-phenyl)methyl-methyl-amino]acetate (1.30 g, 96% yield). ¹H NMR (400 MHz, CDCl₃) 6.81 (d, J=7.9 Hz, 1H), 6.78 (d, J=1.8 Hz, 1H), 6.74 (dd, J=7.9, 1.8 Hz, 1H), 3.71 (s, 3H), 3.52 (s, 2H), 3.22 (s, 2H), 2.28 (s, 3H).

Step C: Preparation of 2-[(2-amino-4-bromo-phenyl)methyl-methyl-amino]acetic acid: To a solution of methyl 2-[(2-amino-4-bromo-phenyl)methyl-methyl-amino]acetate (1.30 g, 4.53 mmol) in THF (15 mL) and water (5 mL) was added lithium hydroxide (0.950 g, 39.7 mmol). The mixture was stirred at rt for 3 h. To the reaction mixture was added 4 N HCl to adjust the pH to 3-4. The reaction mixture was concentrated. The residue was taken up in EtOAc, dried over Na₂SO₄ and filtered. The filtrate was concentrated to dryness to give crude 2-[(2-amino-4-bromo-phenyl)methyl-methyl-amino]acetic acid (1.10 g, 89% yield) which was used in the next step directly.

Step D: Preparation of 8-bromo-4-methyl-3,5-dihydro-1H-1,4-benzodiazepin-2-one: To a solution of 2-[(2-amino-4-bromo-phenyl)methyl-methyl-amino]acetic acid (200 mg, 0.732 mmol), 1-hydroxybenzotriazole (158 mg, 1.17 mmol) and 1-(3-dimethylaminopropyl)-3-ethylcarbodiimide hydrochloride (225 mg, 1.17 mmol) in DMF (10 mL) was added N,N-diisopropylethylamine (0.640 mL, 3.66 mmol). The mixture was stirred at rt for 16 h. To the reaction mixture was added saturated aqueous NaHCO₃ solution, and the mixture was extracted with ethyl acetate. The combined organic layers were washed with saturated brine solution, dried over Na₂SO₄ and concentrated. The residue was purified by preparative TLC (DCM/MeOH=20/1) to give 8-bromo-4-methyl-3,5-dihydro-1H-1,4-benzodiazepin-2-one (50 mg, 27% yield) as a white solid.

Step E: Preparation of 4-methyl-8-(4,4,5,5-tetramethyl-1,3,2-dioxaborolan-2-yl)-3,5-dihydro-1H-1,4-benzodiazepin-2-one: To a stirred solution of 8-bromo-4-methyl-3,5-dihydro-1H-1,4-benzodiazepin-2-one (100 mg, 0.394 mmol), bis(pinacolato)diboron (120 mg, 0.473 mmol) and potassium carbonate (116 mg, 1.18 mmol) in 1,4-dioxane (2 mL) was added 1,1′-bis (di-t-butylphosphino)ferrocene palladium dichloride (29 mg, 0.039 mmol) under Argon. The mixture was stirred at 100° C. for 16 h. The reaction mixture was filtered. The filtrate was concentrated and then diluted with petroleum ether. The mixture was filtered and the filtrated was concentrated in vacuum to give crude 4-methyl-8-(4,4,5,5-tetramethyl-1,3,2-dioxaborolan-2-yl)-3,5-dihydro-1H-1,4-benzodiazepin-2-one (45 mg, 38% yield) as a brown solid. LCMS ESI (+) m/z 303.2 (M+H).

Step F: Preparation of 8-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-4-methyl-1,3,4,5-tetrahydro-2H-benzo[e][1,4]diazepin-2-one: To a stirred solution of (S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate (20 mg, 0.030 mmol) and 4-methyl-8-(4,4,5,5-tetramethyl-1,3,2-dioxaborolan-2-yl)-3,5-dihydro-1H-1,4-benzodiazepin-2-one (11 mg, 0.036 mmol) in 1,4-dioxane (1 mL) and water (0.1 mL) were added Na₂CO₃ (9.7 mg, 0.091 mmol) and Pd(PPh₃)₄(3.5 mg, 0.0030 mmol). The mixture was stirred at 100° C. for 2 h under Argon. The reaction mixture was concentrated in vacuum. The crude product was purified by preparative HPLC to give 8-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-4-methyl-1,3,4,5-tetrahydro-2H-benzo[e][1,4]diazepin-2-one (13 mg, 63% yield). ¹H NMR (400 MHz, CD₃OD) δ 7.80 (d, J=8.0 Hz, 1H), 7.74 (d, J=8.0 Hz, 1H), 7.66-7.72 (m, 2H), 7.57 (d, J=8.0 Hz, 1H), 6.78-6.92 (m, 2H), 6.65-6.74 (m, 1H), 6.23-6.34 (m, 1H), 6.18 (s, 1H), 5.81 (d, J=12.0 Hz, 1H), 5.47-5.74 (m, 1H), 4.13-4.21 (m, 1H), 3.97-4.11 (m, 3H), 3.90 (s, 2H), 3.40-3.45 (m, 2H), 3.38 (s, 2H), 3.03 (s, 3H), 2.61 (s, 3H), 1.35-1.52 (m, 3H). LCMS ESI (+) m/z 685.2 (M+H).

### Synthetic Example 84: Synthesis of 1-((R)-2-((S)-7-(2,4-difluoro-6-(2-methoxyethoxy)phenyl)-4-(8,8-difluoro-6-methyl-5,6,7,8-tetrahydro-1,6-naphthyridin-3-yl)thieno[3,2-c]pyridin-6-yl)-4-methyl-6,7-dihydropyrazolo[1,5-a]pyrazin-5(4H)-yl)prop-2-en-1-one (Compound 1152)

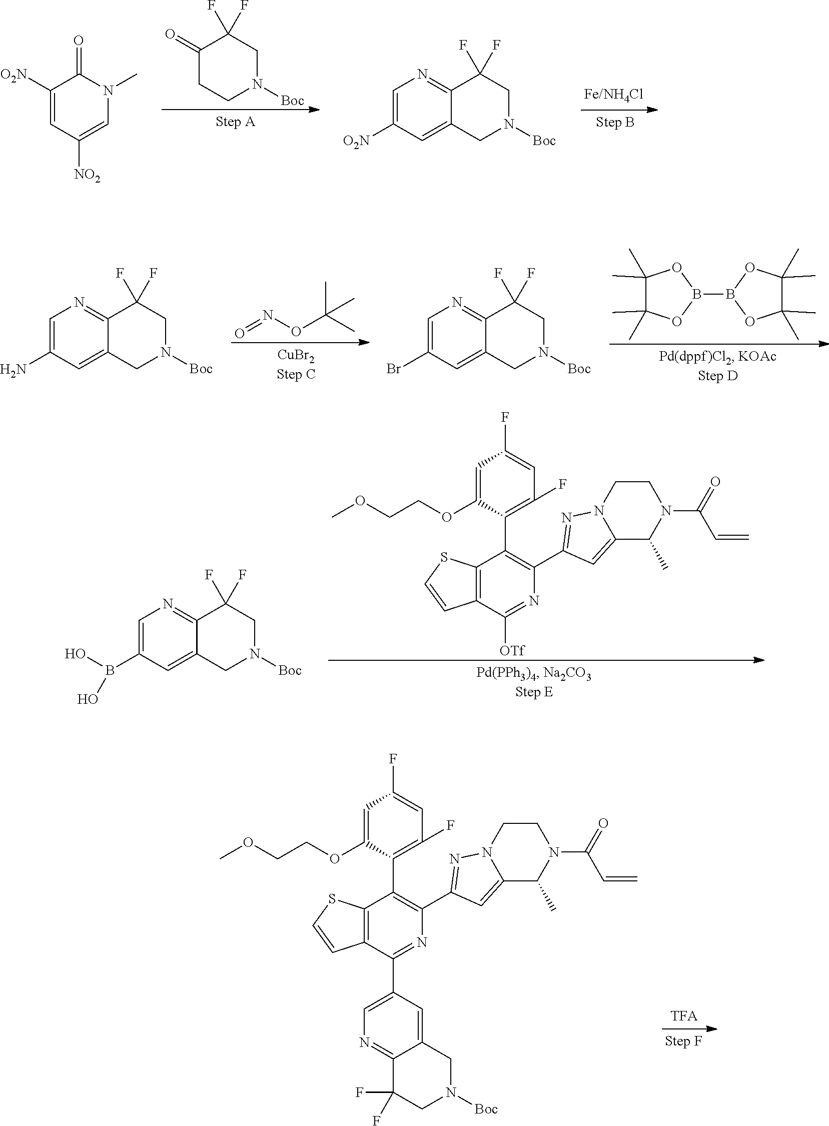

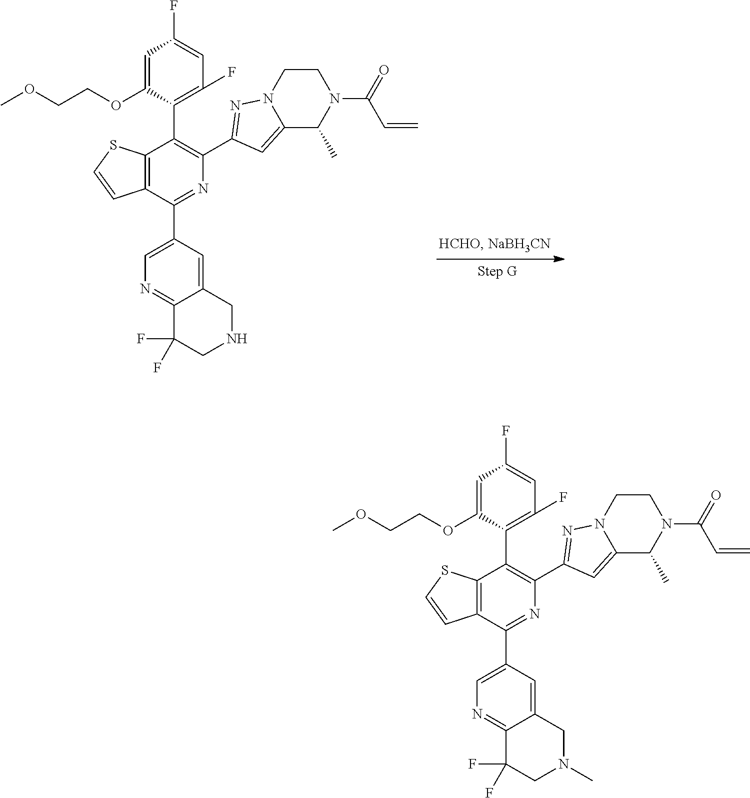

Step A: Preparation of tert-butyl 8,8-difluoro-3-nitro-5,7-dihydro-1,6-naphthyridine-6-carboxylate: To a solution of 1-methyl-3,5-dinitro-pyridin-2-one (1.10 g, 5.52 mmol) and tert-butyl 3,3-difluoro-4-oxo-piperidine-1-carboxylate (1.30 g, 5.52 mmol) in methanol (12 mL) was added aqueous ammonia solution (4.00 mL, 32.0 mmol). The mixture was stirred at 70° C. for 8 h. The reaction mixture was concentrated and purified by column chromatography on silica gel (Petroleum ether/EtOAc=5/1) to give tert-butyl 8,8-difluoro-3-nitro-5,7-dihydro-1,6-naphthyridine-6-carboxylate (180 mg, 10% yield). LCMS ESI (+) m/z 316.1 (M+H).

Step B: Preparation of tert-butyl 3-amino-8,8-difluoro-5,7-dihydro-1,6-naphthyridine-6-carboxylate: To a solution of tert-butyl 8,8-difluoro-3-nitro-5,7-dihydro-1,6-naphthyridine-6-carboxylate (85 mg, 0.27 mmol) in THF/H₂O (6 mL/2 mL) was added Fe (360 mg, 6.74 mmol) and NH₄Cl (376 mg, 6.74 mmol). The mixture was stirred at 65° C. for 4 h. The reaction mixture was cooled to rt and filtered. The filtrate was concentrated and purified by preparative TLC (Petroleum ether/EtOAc=1/1) to give tert-butyl 3-amino-8,8-difluoro-5,7-dihydro-1,6-naphthyridine-6-carboxylate (65 mg, 85% yield). LCMS ESI (+) m/z 286.2 (M+H).

Step C: Preparation of tert-butyl 3-bromo-8,8-difluoro-5,7-dihydro-1,6-naphthyridine-6-carboxylate: To a solution of tert-butyl 3-amino-8,8-difluoro-5,7-dihydro-1,6-naphthyridine-6-carboxylate (65 mg, 0.23 mmol) in acetonitrile (6 mL) was added tert-butyl nitrite (36 mg, 0.34 mmol) and cupric bromide (76 mg, 0.34 mmol). The mixture was stirred at rt for 4 h. The reaction mixture was filtered and the filtrate was concentrated. The residue was purified by preparative TLC (Petroleum ether:EtOAc=3:1) to give tert-butyl 3-bromo-8,8-difluoro-5,7-dihydro-1,6-naphthyridine-6-carboxylate (60 mg, 75% yield). LCMS ESI (+) m/z 349.0 (M+H).

Step D: Preparation of (6-tert-butoxycarbonyl-8,8-difluoro-5,7-dihydro-1,6-naphthyridin-3-yl)boronic acid: To a solution of tert-butyl 3-bromo-8,8-difluoro-5,7-dihydro-1,6-naphthyridine-6-carboxylate (60 mg, 0.17 mmol) and bis(pinacolato)diboron (66 mg, 0.26 mmol) in 1,4-dioxane (4 mL) was added KOAc (34 mg, 0.35 mmol) and Pd(dppf)Cl₂ (13 mg, 0.017 mmol). The reaction mixture was stirred at 95° C. for 4 h under Ar. The reaction mixture was filtered and concentrated. The residue was purified by preparative HPLC to give (6-tert-butoxycarbonyl-8,8-difluoro-5,7-dihydro-1,6-naphthyridin-3-yl)boronic acid (25 mg, 46% yield) as a white solid. LCMS ESI (+) m/z 315.1 (M+H).

Step E: Preparation of tert-butyl 3-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-8,8-difluoro-7,8-dihydro-1,6-naphthyridine-6(5H)-carboxylate: To a solution of (S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl trifluoromethanesulfonate (20 mg, 0.030 mmol) and (6-tert-butoxycarbonyl-8,8-difluoro-5,7-dihydro-1,6-naphthyridin-3-yl)boronic acid (13 mg, 0.040 mmol) in 1,4-dioxane (2 mL)/water (0.2 mL) was added Na₂CO₃ (13 mg, 0.12 mmol) and tetrakis(triphenylphosphine)palladium (3.5 mg, 0.0030 mmol). The reaction mixture was stirred at 100° C. for 2 h under Ar. The reaction mixture was filtered and concentrated. The residue was purified by preparative TLC (DCM:MeOH=20:1) to give tert-butyl 3-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-8,8-difluoro-7,8-dihydro-1,6-naphthyridine-6(5H)-carboxylate (15 mg, 63% yield) as a white solid. LCMS ESI (+) m/z 779.3 (M+H).

Step F: Preparation of 1-((R)-2-((S)-4-(8,8-difluoro-5,6,7,8-tetrahydro-1,6-naphthyridin-3-yl)-7-(2,4-difluoro-6-(2-methoxyethoxy)phenyl)thieno[3,2-c]pyridin-6-yl)-4-methyl-6,7-dihydropyrazolo[1,5-a]pyrazin-5(4H)-yl)prop-2-en-1-one: To a solution tert-butyl 3-((S)-6-((R)-5-acryloyl-4-methyl-4,5,6,7-tetrahydropyrazolo[1,5-a]pyrazin-2-yl)-7-(2,4-difluoro-6-(2-methoxyethoxy)phenyl)thieno[3,2-c]pyridin-4-yl)-8,8-difluoro-7,8-dihydro-1,6-naphthyridine-6(5H)-carboxylate (15 mg, 0.019 mmol) in DCM (3.0 mL) was added trifluoroacetic acid (1.0 mL). The mixture was stirred at rt for 1 h. The solution was concentrated to give crude 1-((R)-2-((S)-4-(8,8-difluoro-5,6,7,8-tetrahydro-1,6-naphthyridin-3-yl)-7-(2,4-difluoro-6-(2-methoxyethoxy)phenyl)thieno[3,2-c]pyridin-6-yl)-4-methyl-6,7-dihydropyrazolo[1,5-a]pyrazin-5(4H)-yl)prop-2-en-1-one (21 mg), which was used for the next step without further purification. LCMS ESI (+) m/z 679.3 (M+H).

Step G: Preparation of 1-((R)-2-((S)-7-(2,4-difluoro-6-(2-methoxyethoxy)phenyl)-4-(8,8-difluoro-6-methyl-5,6,7,8-tetrahydro-1,6-naphthyridin-3-yl)thieno[3,2-c]pyridin-6-yl)-4-methyl-6,7-dihydropyrazolo[1,5-a]pyrazin-5(4H)-yl)prop-2-en-1-one: To a solution of crude 1-((R)-2-((S)-4-(8,8-difluoro-5,6,7,8-tetrahydro-1,6-naphthyridin-3-yl)-7-(2,4-difluoro-6-(2-methoxyethoxy)phenyl)thieno[3,2-c]pyridin-6-yl)-4-methyl-6,7-dihydropyrazolo[1,5-a]pyrazin-5(4H)-yl)prop-2-en-1-one (21 mg) in methanol (2 mL) was added NaOAc (18 mg, 0.22 mmol) to adjust the pH to 5-6. Formaldehyde aqueous solution (1 mL) was added and the solution was stirred at rt for 30 min. Then sodium cyanoborohydride (2.1 mg, 0.033 mmol) was added and the mixture was stirred at rt for 1 h. The reaction mixture was washed with NaHCO₃ saturated aqueous solution and extracted with EtOAc twice. The combined organics were dried over anhydrous Na₂SO₄, filtered and concentrated. The resulting residue was purified by preparative HPLC to give 1-((R)-2-((S)-7-(2,4-difluoro-6-(2-methoxyethoxy)phenyl)-4-(8,8-difluoro-6-methyl-5,6,7,8-tetrahydro-1,6-naphthyridin-3-yl)thieno[3,2-c]pyridin-6-yl)-4-methyl-6,7-dihydropyrazolo[1,5-a]pyrazin-5(4H)-yl)prop-2-en-1-one (8.1 mg, 52% yield) as a bis trifluoracetic acid salt. ¹H NMR (400 MHz, CD₃OD) δ 9.32 (d, J=1.6 Hz, 1H), 8.48 (s, 1H), 7.87 (d, J=5.6 Hz, 1H), 7.74 (d, J=5.6 Hz, 1H), 6.78-6.93 (m, 2H), 6.68-6.76 (m, 1H), 6.28 (d, J=14.2 Hz, 1H), 6.17 (s, 1H), 5.82 (dd, J=1.3, 10.6 Hz, 1H), 5.41-5.76 (m, 1H), 4.53 (s, 2H), 4.17-4.24 (m, 1H), 3.96-4.14 (m, 5H), 3.60-3.90 (m, 1H), 3.41 (t, J=4.6 Hz, 2H), 3.01-3.06 (m, 6H), 1.37-1.53 (m, 3H). LCMS ESI (+) m/z 693.3 (M+H).

### Synthetic Example 85: Synthesis of 1-((6R)-2-(7-(2,4-difluoro-6-(2-methoxyethoxy)phenyl)-4-(3-((dimethylamino)methyl)bicyclo[1.1.1]pentan-1-yl)thieno[3,2-c]pyridin-6-yl)-6-methyl-6,7-dihydropyrazolo[1,5-a]pyrazin-5(4H)-yl)prop-2-en-1-one (Compound 1168)

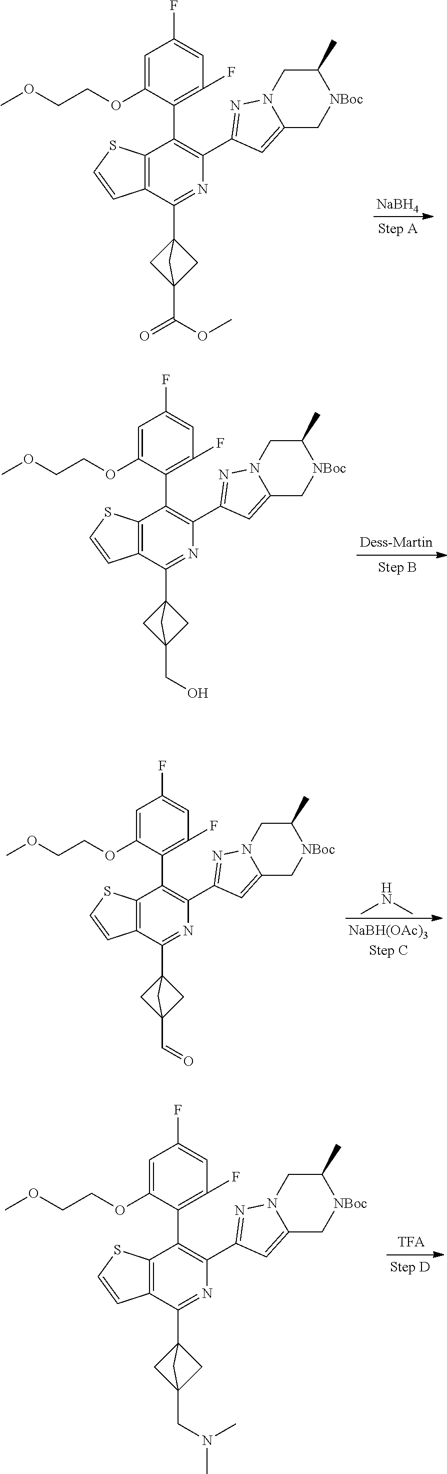

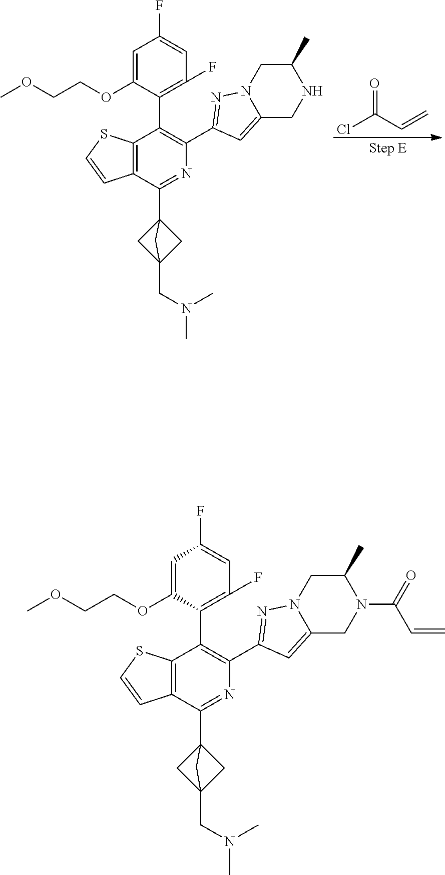

Step A: Preparation of tert-butyl(6R)-2-[7-[2,4-difluoro-6-(2-methoxyethoxy)phenyl]-4-[3-(hydroxymethyl)-1-bicyclo[1.1.1]pentanyl]thieno[3,2-c]pyridin-6-yl]-6-methyl-6,7-dihydro-4H-pyrazolo[1,5-a]pyrazine-5-carboxylate: A mixture of tert-butyl (6R)-2-[7-[2,4-difluoro-6-(2-methoxyethoxy)phenyl]-4-(3-methoxycarbonyl-1-bicyclo[1.1.1]pentanyl)thieno[3,2-c]pyridin-6-yl]-6-methyl-6,7-dihydro-4H-pyrazolo[1,5-a]pyrazine-5-carboxylate (220 mg, 0.323 mmol) and NaBH₄ (37 mg, 0.97 mmol) in THF (8 mL) and methanol (2 mL) was stirred at rt for 12 h. The reaction was concentrated to dryness. The residue was taken up in EtOAc, washed with water and saturated brine solution. The organics were then separated and dried (MgSO₄) before concentration to dryness. The crude was purified by flash column chromatography eluting with (MeOH/DCM=0 to 10%). The desired fractions were concentrated to dryness in vacuo to give tert-butyl(6R)-2-[7-[2,4-difluoro-6-(2-methoxyethoxy)phenyl]-4-[3-(hydroxymethyl)-1-bicyclo[1.1.1]pentanyl]thieno[3,2-c]pyridin-6-yl]-6-methyl-6,7-dihydro-4H-pyrazolo[1,5-a]pyrazine-5-carboxylate (120 mg, 54% yield). LCMS ESI (+) m/z 653.3 (M+H).

Step B: Preparation of tert-butyl (6R)-2-[7-[2,4-difluoro-6-(2-methoxyethoxy)phenyl]-4-(3-formyl-1-bicyclo[1.1.1]pentanyl)thieno[3,2-c]pyridin-6-yl]-6-methyl-6,7-dihydro-4H-pyrazolo[1,5-a]pyrazine-5-carboxylate: A mixture of tert-butyl (6R)-2-[7-[2,4-difluoro-6-(2-methoxyethoxy)phenyl]-4-[3-(hydroxymethyl)-1-bicyclo[1.1.1]pentanyl]thieno[3,2-c]pyridin-6-yl]-6-methyl-6,7-dihydro-4H-pyrazolo[1,5-a]pyrazine-5-carboxylate (160 mg, 0.245 mmol) and Dess-Martin periodinane (312 mg, 0.735 mmol) in DCM (3 mL) was stirred at rt for 2 h. Upon completion of the reaction, the reaction mixture was concentrated in vacuo. The mixture was diluted with water, basified with saturated aqueous NaHCO₃ and extracted with EtOAc. The organic layers were combined, dried over MgSO₄ and concentrated under reduced pressure. The residue was purified by flash column chromatography (MeOH/DCM=0 to 10%) to give tert-butyl (6R)-2-[7-[2,4-difluoro-6-(2-methoxyethoxy)phenyl]-4-(3-formyl-1-bicyclo[1.1.1]pentanyl)thieno[3,2-c]pyridin-6-yl]-6-methyl-6,7-dihydro-4H-pyrazolo[1,5-a]pyrazine-5-carboxylate (70 mg, 44% yield). LCMS ESI (+) m/z 651.0 (M+H).

Step C: Preparation of tert-butyl (6R)-2-(7-(2,4-difluoro-6-(2-methoxyethoxy)phenyl)-4-(3-((dimethylamino)methyl)bicyclo[1.1.1]pentan-1-yl)thieno[3,2-c]pyridin-6-yl)-6-methyl-6,7-dihydropyrazolo[1,5-a]pyrazine-5(4H)-carboxylate: A mixture of tert-butyl (6R)-2-[7-[2,4-difluoro-6-(2-methoxyethoxy)phenyl]-4-(3-formyl-1-bicyclo[1.1.1]pentanyl)thieno[3,2-c]pyridin-6-yl]-6-methyl-6,7-dihydro-4H-pyrazolo[1,5-a]pyrazine-5-carboxylate (70 mg, 0.11 mmol) and dimethylamine (0.016 mL, 0.32 mmol) in DCM (5 mL) was stirred at rt for 30 min. NaBH(OAc)₃ (68 mg, 0.32 mmol) was added. The mixture was stirred at rt for 12 h. Upon completion of the reaction, the reaction mixture was concentrated in vacuo. The mixture was diluted with water, basified with saturated aqueous NaHCO₃ solution and extracted with EtOAc. The organic layers were combined, dried over MgSO₄ and concentrated under reduced pressure. The residue was purified by flash column chromatography (MeOH/DCM=0 to 10%) to give tert-butyl (6R)-2-(7-(2,4-difluoro-6-(2-methoxyethoxy)phenyl)-4-(3-((dimethylamino)methyl)bicyclo[1.1.1]pentan-1-yl)thieno[3,2-c]pyridin-6-yl)-6-methyl-6,7-dihydropyrazolo[1,5-a]pyrazine-5(4H)-carboxylate (35 mg, 47% yield). LCMS ESI (+) m/z 680.2 (M+H).

Step D: Preparation of 1-(3-(7-(2,4-difluoro-6-(2-methoxyethoxy)phenyl)-6-((R)-6-methyl-4,5,6,7-tetrahydropyrazolo[1,5-a]pyrazin-2-yl)thieno[3,2-c]pyridin-4-yl)bicyclo[1.1.1]pentan-1-yl)-N,N-dimethylmethanamine: A mixture of tert-butyl (6R)-2-[7-[2,4-difluoro-6-(2-methoxyethoxy)phenyl]-4-[3-[(dimethylamino)methyl]-1-bicyclo[1.1.1]pentanyl]thieno[3,2-c]pyridin-6-yl]-6-methyl-6,7-dihydro-4H-pyrazolo[1,5-a]pyrazine-5-carboxylate (35 mg, 0.052 mmol) and TFA (0.059 mL, 0.77 mmol) in DCM (3 mL) was stirred at 25° C. for 2 h. Upon completion of the reaction, The resulting mixture was concentrated to give crude 1-(3-(7-(2,4-difluoro-6-(2-methoxyethoxy)phenyl)-6-((R)-6-methyl-4,5,6,7-tetrahydropyrazolo[1,5-a]pyrazin-2-yl)thieno[3,2-c]pyridin-4-yl)bicyclo[1.1.1]pentan-1-yl)-N,N-dimethylmethanamine, which was used in next step directly. LCMS ESI (+) m/z 580.2 (M+H).

Step E: Preparation of 1-((6R)-2-(7-(2,4-difluoro-6-(2-methoxyethoxy)phenyl)-4-(3-((dimethylamino)methyl)bicyclo[1.1.1]pentan-1-yl)thieno[3,2-c]pyridin-6-yl)-6-methyl-6,7-dihydropyrazolo[1,5-a]pyrazin-5(4H)-yl)prop-2-en-1-one: To a stirred solution of 1-[3-[7-[2,4-difluoro-6-(2-methoxyethoxy)phenyl]-6-[(6R)-6-methyl-4,5,6,7-tetrahydropyrazolo[1,5-a]pyrazin-2-yl]thieno[3,2-c]pyridin-4-yl]-1-bicyclo[1.1.1]pentanyl]-N,N-dimethyl-methanamine (30 mg, 0.052 mmol) and DIEA (0.028 mL, 0.16 mmol) in DCM (3 mL) was stirred at 0° C. for 2 h. Upon completion of the reaction, the reaction mixture was concentrated in vacuo. The mixture was diluted with water, basified with saturated aqueous NaHCO₃ solution and extracted with EtOAc. The organic layers were combined, dried over MgSO₄ and concentrated under reduced pressure. The crude product was purified by preparative HPLC (Gemini-C18 150×21.2 mm, 5 um, ACN-H₂O (0.1% FA) gradient 20-50%) to give 1-((6R)-2-(7-(2,4-difluoro-6-(2-methoxyethoxy)phenyl)-4-(3-((dimethylamino)methyl)bicyclo[1.1.1]pentan-1-yl)thieno[3,2-c]pyridin-6-yl)-6-methyl-6,7-dihydropyrazolo[1,5-a]pyrazin-5(4H)-yl)prop-2-en-1-one (4.8 mg, 12% yield) as a trifluoroacetic acid salt. ¹H NMR (400 MHz, CD₃OD) δ 7.97 (s, 2H), 7.02-6.67 (m, 3H), 6.30 (dd, J=16.8, 1.3 Hz, 1H), 6.16 (s, 1H), 5.88-5.79 (m, 1H), 5.40-5.08 (m, 2H), 4.75-4.15 (m, 4H), 4.14-3.87 (m, 2H), 3.52 (s, 2H), 3.42 (dt, J=6.6, 4.6 Hz, 2H), 3.11-2.96 (m, 8H), 2.79-2.57 (m, 6H), 1.21 (d, J=7.0 Hz, 3H). LCMS ESI (+) m/z 634.0 (M+H).

### Synthetic Example 86: Synthesis of 5-((R)-3-((R)-5-acryloyl-4-methyl-4,5,6,7-tetrahydropyrazolo[1,5-a]pyrazin-2-yl)-4-(2,4-difluoro-6-(2-methoxyethoxy)phenyl)-6,7-dihydro-5H-cyclopenta[c]pyridin-1-yl)isoindolin-1-one (Compound 1174)

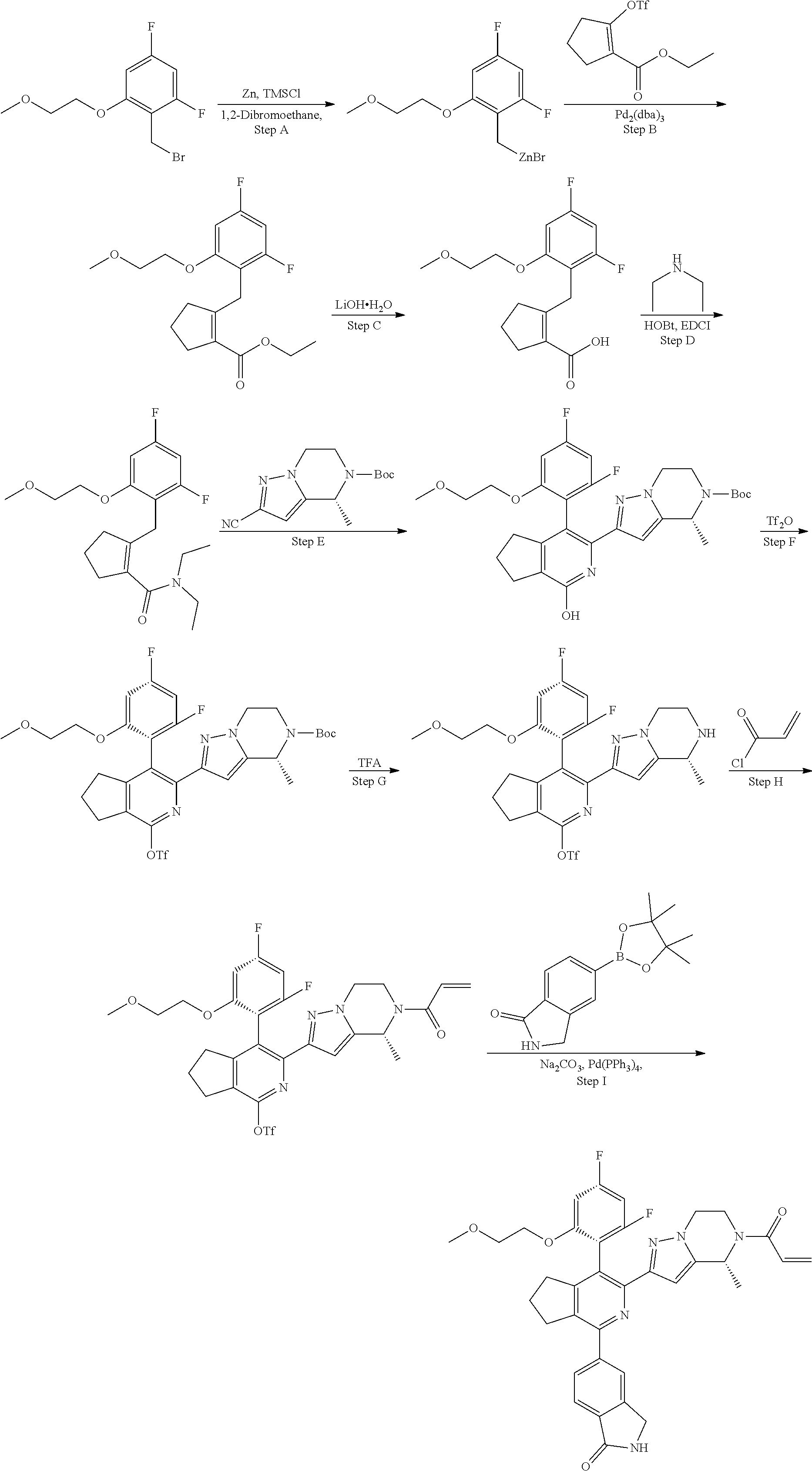

Step A: Preparation of (2,4-difluoro-6-(2-methoxyethoxy)benzyl)zinc(II) bromide: A mixture of Zn (11.9 g, 184 mmol) and 1,2-dibromoethane (1.72 g, 9.18 mmol) in DMF (80 mL) was stirred at 80° C. for 20 min. TMSCl (166 mg, 1.53 mmol) was added at rt. The resulting mixture was stirred at rt for 30 min. A solution of 2-(bromomethyl)-1,5-difluoro-3-(2-methoxyethoxy)benzene (4.30 g, 15.3 mmol) in DMF was added. The mixture was stirred at rt for 1 h and then used in next step directly.

Step B: Preparation of ethyl 2-(2,4-difluoro-6-(2-methoxyethoxy)benzyl)cyclopent-1-ene-1-carboxylate: A mixture of bromo-[[2,4-difluoro-6-(2-methoxyethoxy)phenyl]methyl]zinc (7.95 g, 22.9 mmol), ethyl 2-(trifluoromethylsulfonyloxy)cyclopentene-1-carboxylate (4.41 g, 15.3 mmol), tri(o-tolyl)phosphine (465 mg, 1.53 mmol) and Pd₂(dba)₃ (1.40 g, 1.53 mmol) in DMF (80 mL) was stirred at rt for 12 h. Upon completion of the reaction, water (100 mL) was added. The resulting mixture was filtered. The filtrate was poured into water and extracted with EtOAc. The organic layers were combined, dried over MgSO₄ and concentrated under reduced pressure. The residue was purified by flash column chromatography on silica gel (EtOAc/petroleum ether=0 to 20%) to give ethyl 2-(2,4-difluoro-6-(2-methoxyethoxy)benzyl)cyclopent-1-ene-1-carboxylate (4.10 g, 75% yield). LCMS ESI (+) m/z 363.1 (M+H).

Step C: Preparation of tert-butyl (6R)-2-(7-(2,4-difluoro-6-(2-methoxyethoxy)phenyl)-4-(3-((dimethylamino)methyl)bicyclo[1.1.1]pentan-1-yl)thieno[3,2-c]pyridin-6-yl)-6-methyl-6,7-dihydropyrazolo[1,5-a]pyrazine-5(4H)-carboxylate: A mixture of ethyl 2-[[2,4-difluoro-6-(2-methoxyethoxy)phenyl]methyl]cyclopentene-1-carboxylate (4.00 g, 11.8 mmol) and LiOH·H₂O (4.93 g, 118 mmol) in 1,4-dioxane (32 mL) and water (8 mL) was stirred at rt for 12 h. The pH was adjusted to 2 by adding 1 N HCl solution, The aqueous layer was extracted with EtOAc to give crude 2-[[2,4-difluoro-6-(2-methoxyethoxy)phenyl]methyl]cyclopentene-1-carboxylic acid (3.00 g, 78% yield). LCMS ESI (+) m/z 335.0 (M+Na).

Step D: Preparation of 2-(2,4-difluoro-6-(2-methoxyethoxy)benzyl)-N,N-diethylcyclopent-1-ene-1-carboxamide: A solution of diethylamine (3.00 mL, 28.8 mmol), 2-[[2,4-difluoro-6-(2-methoxyethoxy)phenyl]methyl]cyclopentene-1-carboxylic acid (6.00 g, 19.2 mmol), EDCI (7.37 g, 38.4 mmol), HOBT (5.18 g, 38.4 mmol) and TEA (13.0 mL, 96.1 mmol) in DMF (150 mL) was stirred at 25° C. for 16 h, Upon completion of the reaction, the resulting mixture was concentrated and partitioned between EtOAc and water. The organic layers were combined, dried over MgSO₄ and concentrated under reduced pressure. The crude product was chromatographed on silica gel (EtOAc/petroleum ether=0 to 50%) to give 2-(2,4-difluoro-6-(2-methoxyethoxy)benzyl)-N,N-diethylcyclopent-1-ene-1-carboxamide (4.80 g, 65% yield). LCMS ESI (+) m/z 368.1 (M+H).

Step E: Preparation of tert-butyl (4R)-2-(4-(2,4-difluoro-6-(2-methoxyethoxy)phenyl)-1-hydroxy-6,7-dihydro-5H-cyclopenta[c]pyridin-3-yl)-4-methyl-6,7-dihydropyrazolo[1,5-a]pyrazine-5(4H)-carboxylate: To a stirred solution of 2-[[2,4-difluoro-6-(2-methoxvethoxy)phenyl]methyl]-N,N-diethyl-cyclopentene-1-carboxamide (924 mg, 2.52 mmol) in THF (6 mL) was added n-BuLi (2.5 M in hexanes, 1.10 mL, 2.74 mmol) at −60° C. The mixture was stirred at −60° C. for 20 min, and then a solution of tert-butyl (4R)-2-cyano-4-methyl-6,7-dihydro-4H-pyrazolo[1,5-a]pyrazine-5-carboxylate (600 mg, 2.29 mmol) in THF (0.5 mL) was added. The resulting mixture was stirred at rt for 3 h. Upon completion of the reaction, the resulting mixture was poured into water. The reaction was acidified by 1 N HCl solution and extracted with EtOAc. The organic layers were combined, dried over MgSO₄ and concentrated under reduced pressure. The residue was purified by flash column chromatography on silica gel (MeOH/DCM=0 to 10%) to give tert-butyl (4R)-2-(4-(2,4-difluoro-6-(2-methoxyethoxy)phenyl)-1-hydroxy-6,7-dihydro-5H-cyclopenta[c]pyridin-3-yl)-4-methyl-6,7-dihydropyrazolo[1,5-a]pyrazine-5(4H)-carboxylate (260 mg, 18% yield). LCMS ESI (+) m/z 557.1 (M+H).

Step F: Preparation of tert-butyl (R)-2-((R)-4-(2,4-difluoro-6-(2-methoxyethoxy)phenyl)-1-(((trifluoromethyl)sulfonyl)oxy)-6,7-dihydro-5H-cyclopenta[c]pyridin-3-yl)-4-methyl-6,7-dihydropyrazolo[1,5-a]pyrazine-5(4H)-carboxylate: To a solution of tert-butyl (4R)-2-[4-[2,4-difluoro-6-(2-methoxyethoxy)phenyl]-1-hydroxy-6,7-dihydro-5H-cyclopenta[c]pyridin-3-yl]-4-methyl-6,7-dihydro-4H-pyrazolo[1,5-a]pyrazine-5-carboxylate (220 mg, 0.395 mmol) and pyridine (0.430 mL, 3.95 mmol) in DCM (5 mL) was added trifluoromethanesulfonic anhydride (0.100 mL, 0.593 mmol). The mixture was stirred at 25° C. for 1 h. The mixture was concentrated and diluted with EtOAc. The mixture was washed with brine, dried over anhydrous Na₂SO₄ and concentrated. The residue was purified by column chromatography on silica gel (EtOAc/petroleum ether: 1/8) to separate the two diastereomers. A diastereomer tert-butyl (R)-2-((R)-4-(2,4-difluoro-6-(2-methoxyethoxy)phenyl)-1-(((trifluoromethyl)sulfonyl)oxy)-6,7-dihydro-5H-cyclopenta[c]pyridin-3-yl)-4-methyl-6,7-dihydropyrazolo[1,5-a]pyrazine-5(4H)-carboxylate (50 mg) was obtained in 18% yield. LCMS ESI (+) m/z 689.2 (M+H).

Step G: Preparation of (R)-4-(2,4-difluoro-6-(2-methoxyethoxy)phenyl)-3-((R)-4-methyl-4,5,6,7-tetrahydropyrazolo[1,5-a]pyrazin-2-yl)-6,7-dihydro-5H-cyclopenta[c]pyridin-1-yl trifluoromethanesulfonate: A mixture of tert-butyl (R)-2-((R)-4-(2,4-difluoro-6-(2-methoxyethoxy)phenyl)-1-(((trifluoromethyl)sulfonyl)oxy)-6,7-dihydro-5H-cyclopenta[c]pyridin-3-yl)-4-methyl-6,7-dihydropyrazolo[1,5-a]pyrazine-5(4H)-carboxylate (70 mg, 0.10 mmol) and TFA (0.39 mL) was stirred in DCM (2 mL) at 25° C. for 1 h. The reaction was concentrated to give crude (R)-4-(2,4-difluoro-6-(2-methoxyethoxy)phenyl)-3-((R)-4-methyl-4,5,6,7-tetrahydropyrazolo[1,5-a]pyrazin-2-yl)-6,7-dihydro-5H-cyclopenta[c]pyridin-1-yl trifluoromethanesulfonate (70 mg, 94% yield), which was used in the next step without further purification. LCMS ESI (+) m/z 589.1 (M+H).

Step H: Preparation of (R)-3-((R)-5-acryloyl-4-methyl-4,5,6,7-tetrahydropyrazolo[1,5-a]pyrazin-2-yl)-4-(2,4-difluoro-6-(2-methoxyethoxy)phenyl)-6,7-dihydro-5H-cyclopenta[c]pyridin-1-yl trifluoromethanesulfonate: To a solution of (R)-4-(2,4-difluoro-6-(2-methoxyethoxy)phenyl)-3-((R)-4-methyl-4,5,6,7-tetrahydropyrazolo[1,5-a]pyrazin-2-yl)-6,7-dihydro-5H-cyclopenta[c]pyridin-1-yl trifluoromethanesulfonate (70 mg, 0.12 mmol) and DIEA (0.050 mL, 0.36 mmol) in DCM (5 mL) was added acryloyl chloride (0.014 mL, 0.18 mmol). The resulting mixture was stirred at −60° C. for 1 hour. The reaction mixture was poured into saturated NaHCO₃ (30 mL). The mixture was extracted with DCM. The organic layer was washed with water and brine, dried over anhydrous sodium sulfate, filtered and concentrated. The crude was purified by flash column chromatography on silica gel eluting with 50% EtOAc in hexanes to give (R)-3-((R)-5-acryloyl-4-methyl-4,5,6,7-tetrahydropyrazolo[1,5-a]pyrazin-2-yl)-4-(2,4-difluoro-6-(2-methoxyethoxy)phenyl)-6,7-dihydro-5H-cyclopenta[c]pyridin-1-yl trifluoromethane sulfonate (40 mg, 52% yield). LCMS ESI (+) m/z 643.2 (M+H).

Step I: Preparation of 5-((R)-3-((R)-5-acryloyl-4-methyl-4,5,6,7-tetrahydropyrazolo[1,5-a]pyrazin-2-yl)-4-(2,4-difluoro-6-(2-methoxyethoxy)phenyl)-6,7-dihydro-5H-cyclopenta[c]pyridin-1-yl)isoindolin-1-one: A mixture of (R)-3-((R)-5-acryloyl-4-methyl-4,5,6,7-tetrahydropyrazolo[1,5-a]pyrazin-2-yl)-4-(2,4-difluoro-6-(2-methoxyethoxy)phenyl)-6,7-dihydro-5H-cyclopenta[c]pyridin-1-yl trifluoromethane sulfonate (30 mg, 0.047 mmol), 5-(4,4,5,5-tetramethyl-1,3,2-dioxaborolan-2-yl)isoindolin-1-one (12 mg, 0.047 mmol), Na₂CO₃ (15 mg, 0.14 mmol) and Pd(PPh₃)₄(8.1 mg, 0.0070 mmol) was stirred in 1,4-dioxane (5 mL) and water (0.5 mL) at 100° C. under N₂ for 1 h. The reaction was concentrated to dryness. The crude was purified by flash column chromatography eluting with 5% MeOH in DCM followed by preparative TLC to give 5-((R)-3-((R)-5-acryloyl-4-methyl-4,5,6,7-tetrahydropyrazolo[1,5-a]pyrazin-2-yl)-4-(2,4-difluoro-6-(2-methoxyethoxy)phenyl)-6,7-dihydro-5H-cyclopenta[c]pyridin-1-yl)isoindolin-1-one (6.9 mg, 24% yield). ¹H NMR (400 MHz, CD₃OD) δ 8.01 (s, 1H), 7.92 (d, J=8.0 Hz, 1H), 7.81 (d, J=7.9 Hz, 1H), 6.79-6.64 (m, 2H), 6.55-6.45 (m, 1H), 6.17 (d, J=13.8 Hz, 1H), 5.93 (s, 1H), 5.76-5.67 (m, 1H), 5.60-5.20 (m, 1H), 4.47 (s, 2H), 4.37-4.16 (m, 1H), 4.11-3.83 (m, 4H), 3.73-3.50 (m, 1H), 3.43 (t, J=4.5 Hz, 2H), 3.17-3.04 (m, 5H), 2.85-2.55 (m, 2H), 2.10-1.93 (m, 2H), 1.41-1.25 (m, 3H). LCMS ESI (+) m/z 626.3 (M+H).

### Synthetic Example 87: Synthesis of (S)-1-(2-(7-(2,4-difluoro-6-(2-hydroxyethoxy)phenyl)-4-(1-methyl-1H-indazol-5-yl)thieno[3,2-c]pyridin-6-yl)-4,5,7,8-tetrahydro-6H-pyrazolo[1,5-d][1,4]diazepin-6-yl)prop-2-en-1-one (Compound 1207)

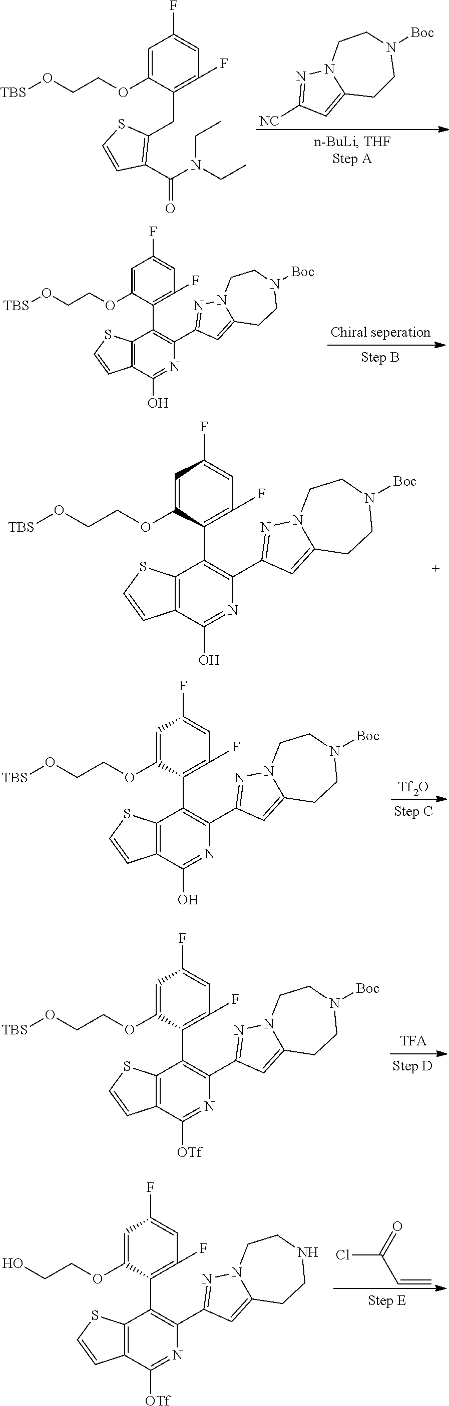

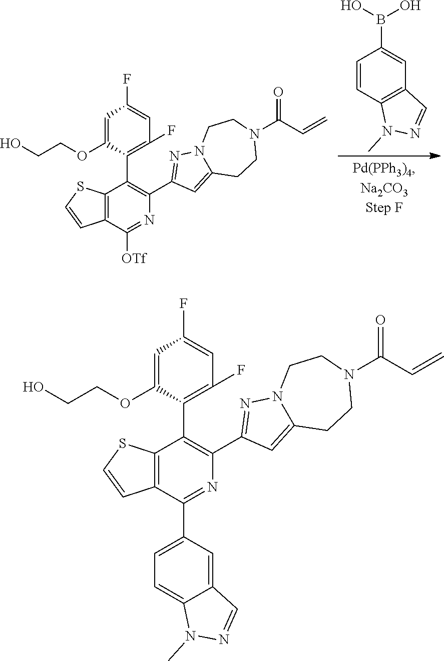

Step A: Preparation of tert-butyl 2-[7-[2-[2-[tert-butyl(dimethyl)silyl]oxyethoxy]-4,6-difluoro-phenyl]-4-hydroxy-thieno[3,2-c]pyridin-6-yl]-4,5,7,8-tetrahydropyrazolo[1,5-d][1,4]diazepine-6-carboxylate: To a stirred solution of 2-[[2-[2-[tert-butyl(dimethyl)silyl]oxyethoxy]-4,6-difluoro-phenyl]methyl]-N,N-diethyl-thiophene-3-carboxamide (720 mg, 1.49 mmol) in THF (5 mL) at −60° C. under Argon was added n-BuLi (2.5 M in hexanes, 9.20 mL, 23.0 mmol). The solution was stirred at −60° C. for 30 min. tert-butyl 2-Cyano-4,5,7,8-tetrahydropyrazolo[1,5-d][1,4]diazepine-6-carboxylate (300 mg, 1.14 mmol) was added. The solution was stirred at 20° C. for 3 h. 1 N HCl aqueous solution was added to adjust the pH to 4. The mixture was diluted with water and extracted with ethyl acetate. The organic layer was washed with saturated brine solution, dried and concentrated. The residue was purified by flash chromatography on silica gel (petroleum ether/EtOAc=1/1 to EA) to give tert-butyl 2-[7-[2-[2-[tert-butyl(dimethyl)silyl]oxyethoxy]-4,6-difluoro-phenyl]-4-hydroxy-thieno[3,2-c]pyridin-6-yl]-4,5,7,8-tetrahydropyrazolo[1,5-d][1,4]diazepine-6-carboxylate (800 mg, 78% yield). ¹HNMR (300 MHz, CD₃OD) δ 7.63 (d, J=5.4 Hz, 1H), 7.49 (d, J=5.4 Hz, 1H), 6.90 (d, J=10.8 Hz, 1H), 6.77 (td, J=2.1, 9.0 Hz, 1H), 5.46 (s, 1H), 4.42 (d, J=5.7 Hz, 2H), 3.97-4.01 (m, 2H), 3.69-3.72 (m, 4H), 3.55 (s, 2H), 2.78-2.81 (m, 2H), 1.50 (s, 9H), 0.69 (s, 9H), 0.25 (d, J=3.9 Hz, 6H).

Step B: Preparation of tert-butyl (S)-2-(7-(2-(2-((tert-butyldimethylsilyl)oxy)ethoxy)-4,6-difluorophenyl)-4-hydroxythieno[3,2-c]pyridin-6-yl)-4,5,7,8-tetrahydro-6H-pyrazolo[1,5-d][1,4]diazepine-6-carboxylate: 749 mg of tert-butyl 2-[7-[2-[2-[tert-butyl(dimethyl)silyl]oxyethoxy]-4,6-difluoro-phenyl]-4-hydroxy-thieno[3,2-c]pyridin-6-yl]-4,5,7,8-tetrahydropyrazolo[1,5-d][1,4]diazepine-6-carboxylate was separated by chiral SFC (CHIRALPAK 1H, 3 cm×25 cm, 5 m, CO₂:MeOH (0.1% 2 mM NH₃-MeOH)=60:40) to give tert-butyl (R)-2-(7-(2-(2-((tert-butyldimethylsilyl)oxy)ethoxy)-4,6-difluorophenyl)-4-hydroxythieno[3,2-c]pyridin-6-yl)-4,5,7,8-tetrahydro-6H-pyrazolo[1,5-d][1,4]diazepine-6-carboxylate (302 mg) as the first peak, and tert-butyl (S)-2-(7-(2-(2-((tert-butyldimethylsilyl)oxy)ethoxy)-4,6-difluorophenyl)-4-hydroxythieno[3,2-c]pyridin-6-yl)-4,5,7,8-tetrahydro-6H-pyrazolo[1,5-d][1,4]diazepine-6-carboxylate (279 mg) as the second peak.

Step C: Preparation of tert-butyl (S)-2-(7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4,5,7,8-tetrahydro-6H-pyrazolo[1,5-d][1,4]diazepine-6-carboxylate: To a solution of tert-butyl (S)-2-(7-(2-(2-((tert-butyldimethylsilyl)oxy)ethoxy)-4,6-difluorophenyl)-4-hydroxythieno[3,2-c]pyridin-6-yl)-4,5,7,8-tetrahydro-6H-pyrazolo[1,5-d][1,4]diazepine-6-carboxylate (279 mg, 0.415 mmol) and pyridine (0.17 mL, 2.1 mmol) in DCM (10 mL) was added trifluoromethanesulfonic anhydride (0.14 mL, 0.83 mmol). The mixture was stirred at 25° C. for 1 h. The mixture was poured into water and the product was extracted with DCM. The organics were washed with brine, dried over anhydrous Na₂SO₄, concentrated and purified by column chromatography on silica gel eluting with 20% EtOAc in petroleum ether to give tert-butyl (S)-2-(7-(2-(2-((tert-butyldimethylsilyl)oxy)ethoxy)-4,6-difluorophenyl)-4-(((trifluoromethyl)sulfonyl) oxy)thieno[3,2-c]pyridin-6-yl)-4,5,7,8-tetrahydro-6H-pyrazolo[1,5-d][1,4]diazepine-6-carboxylate (312 mg, 93% yield).

Step D: Preparation of (S)-7-(2,4-difluoro-6-(2-hydroxyethoxy)phenyl)-6-(5,6,7,8-tetrahydro-4H-pyrazolo[1,5-d][1,4]diazepin-2-yl)thieno[3,2-c]pyridin-4-yl trifluoromethanesulfonate: tert-butyl (S)-2-(7-(2-(2-((tert-butyldimethylsilyl)oxy)ethoxy)-4,6-difluorophenyl)-4-(((trifluoromethyl)sulfonyl)oxy)thieno[3,2-c]pyridin-6-yl)-4,5,7,8-tetrahydro-6H-pyrazolo[1,5-d][1,4]diazepine-6-carboxylate (312 mg, 0.388 mmol) was dissolved in DCM (3 mL). Trifluoroacetic acid (1.0 mL) was added. The resulting mixture was stirred at rt for 1 h. The mixture was concentrated under reduced pressure to afford crude (S)-7-(2,4-difluoro-6-(2-hydroxyethoxy)phenyl)-6-(5,6,7,8-tetrahydro-4H-pyrazolo[1,5-d][1,4]diazepin-2-yl)thieno[3,2-c]pyridin-4-yl trifluoromethanesulfonate (365 mg, 100% yield) which was used in the next step without further purification. LCMS ESI (+) m/z 591.2 (M+H).

Step E: Preparation of (S)-6-(6-acryloyl-5,6,7,8-tetrahydro-4H-pyrazolo[1,5-d][1,4]diazepin-2-yl)-7-(2,4-difluoro-6-(2-hydroxyethoxy)phenyl)thieno[3,2-c]pyridin-4-yl trifluoromethanesulfonate: To a solution of (S)-7-(2,4-difluoro-6-(2-hydroxyethoxy)phenyl)-6-(5,6,7,8-tetrahydro-4H-pyrazolo[1,5-d][1,4]diazepin-2-yl)thieno[3,2-c]pyridin-4-yl trifluoromethanesulfonate (365 mg, 0.388 mmol) in ethyl acetate (5 mL) and water (5 mL) was added dropwise sodium carbonate (174 mg, 2.07 mmol). The reaction mixture was stirred at rt for 10 mins. A solution of acryloyl chloride (0.034 mL, 0.41 mmol) in DCM was added to the mixture dropwise at 0° C., and the mixture was stirred at 0° C. for 30 mins. The reaction was extracted with EtOAc. The combined organics were dried (Na₂SO₄) before concentration to dryness to give crude (S)-6-(6-acryloyl-5,6,7,8-tetrahydro-4H-pyrazolo[1,5-d][1,4]diazepin-2-yl)-7-(2,4-difluoro-6-(2-hydroxyethoxy)phenyl)thieno[3,2-c]pyridin-4-yl trifluoromethanesulfonate (265 mg, 95% yield). LCMS ESI (+) m/z 645.2 (M+H).

Step F: Preparation of (S)-1-(2-(7-(2,4-difluoro-6-(2-hydroxyethoxy)phenyl)-4-(1-methyl-1H-indazol-5-yl)thieno[3,2-c]pyridin-6-yl)-4,5,7,8-tetrahydro-6H-pyrazolo[1,5-d][1,4]diazepin-6-yl)prop-2-en-1-one: To a solution of [(S)-6-(6-acryloyl-5,6,7,8-tetrahydro-4H-pyrazolo[1,5-d][1,4]diazepin-2-yl)-7-(2,4-difluoro-6-(2-hydroxyethoxy)phenyl)thieno[3,2-c]pyridin-4-yl trifluoromethanesulfonate (25 mg, 0,039 mmol), (1-methylindazol-5-yl)boronic acid (8.2 mg, 0.046 mmol) and Na₂CO₃ (12 mg, 0.12 mmol) in 1,4-dioxane (2 mL) and water (0.2 mL) was added Pd(PPh₃)₄(2.8 mg, 0.0039 mmol). The mixture was stirred at 100° C. for 4 h under Ar. The mixture was poured into water and the product was extracted with EtOAc. The combined organics were washed with brine, dried over anhydrous Na₂SO₄, concentrated and purified by preparative HPLC to give (S)-1-(2-(7-(2,4-difluoro-6-(2-hydroxyethoxy)phenyl)-4-(1-methyl-1H-indazol-5-yl)thieno[3,2-c]pyridin-6-yl)-4,5,7,8-tetrahydro-6H-pyrazolo[1,5-d][1,4]diazepin-6-yl)prop-2-en-1-one (13 mg, 54% yield) as a trifluoracetic acid salt. ¹H NMR (400 MHz, CD₃OD) δ 8.49 (s, 1H), 8.28 (s, 1H), 8.00-8.14 (m, 2H), 7.82-7.99 (m, 2H), 6.98 (d, J=10.9 Hz, 1H), 6.76-6.94 (m, 2H), 6.27 (dd, J=16.7, 1.8 Hz, 1H), 6.01 (d, J=11.0 Hz, 1H), 5.81 (dd, J=10.6, 1.7 Hz, 1H), 4.43-4.51 (m, 2H), 4.20 (s, 3H), 3.70-4.10 (m, 6H), 3.58 (t, J=4.7 Hz, 2H), 2.86-3.05 (m, 2H). LCMS ESI (+) m/z 627.3 (M+H).

### Synthetic Example 88: Synthesis of 1-[3-[4-[2,4-difluoro-6-(2-methoxyethoxy)phenyl]-7-(1-methylindazol-5-yl)thieno[2,3-c]pyridin-5-yl]-7,8-dihydro-5H-1,6-naphthyridin-6-yl]prop-2-en-1-one (Compound 1261)

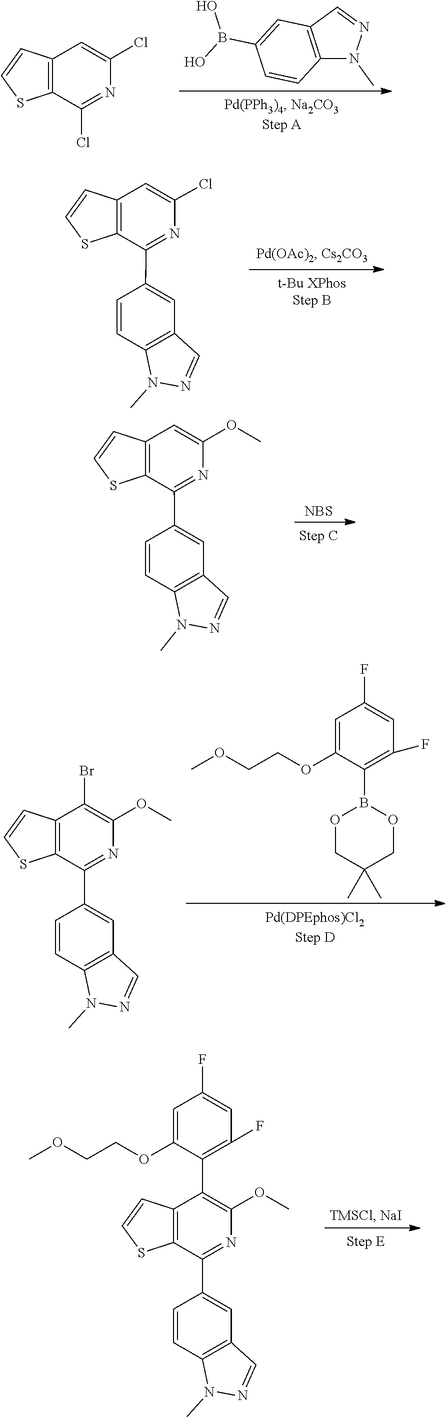

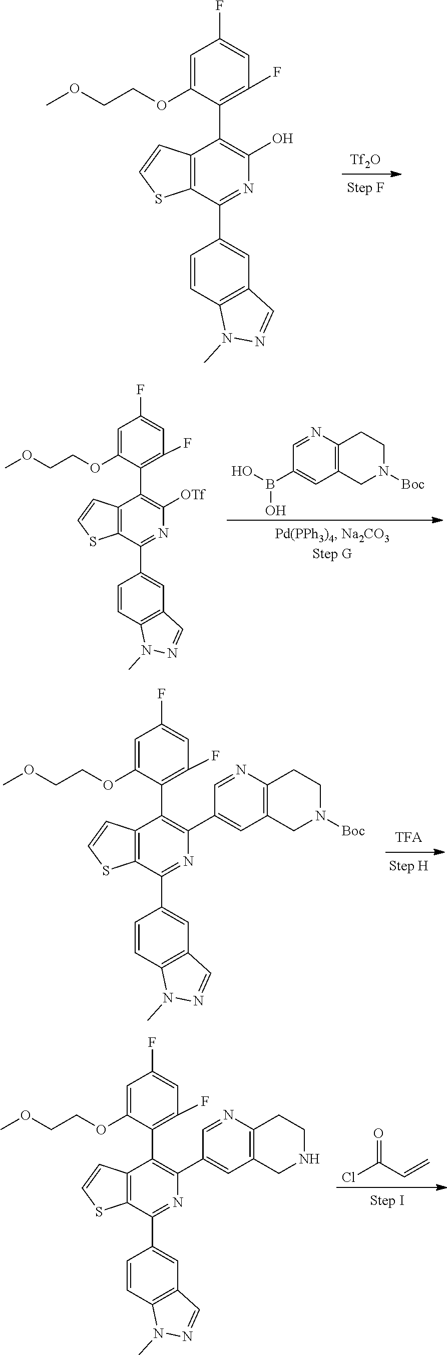

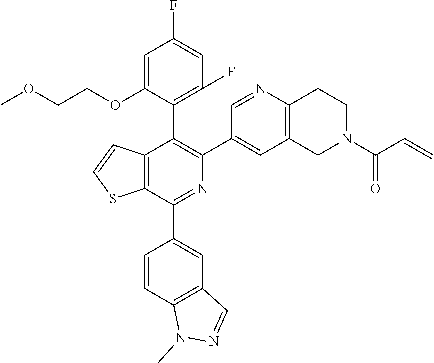 [SMILES: C=CC(=O)N1CCc2ncc(-c3nc(-c4ccc5c(cnn5C)c4)c4sccc4c3-c3c(F)cc(F)cc3OCCOC)cc2C1]

Step A: Preparation of 5-chloro-7-(1-methylindazol-5-yl)thieno[2,3-c]pyridine: A solution of 5,7-dichlorothieno[2,3-c]pyridine (1.00 g, 4.90 mmol), (1-methylindazol-5-yl)boronic acid (1.12 g, 6.37 mmol), tetrakis(triphenylphosphine)palladium(0) (566 mg, 0.490 mmol) and sodium carbonate (1.04 g, 9.80 mmol) in 1,4-dioxane (20 mL)/water (2 mL) was stirred at 95° C. under N₂ for 2 hours. The mixture was diluted with EtOAc, washed with brine solution, dried over Na₂SO₄ and concentrated. The residue was purified by column chromatography (petroleum ether:EtOAc=5:1 to 2:1) to give 5-chloro-7-(1-methylindazol-5-yl)thieno[2,3-c]pyridine (707 mg, 48% yield). LCMS ESI (+) m/z 300.0 (M+H).

Step B: Preparation of 5-methoxy-7-(1-methylindazol-5-yl)thieno[2,3-c]pyridine: A solution of 5-chloro-7-(1-methylindazol-5-yl)thieno[2,3-c]pyridine (707 mg, 2.36 mmol), palladium(II) acetate (53 mg, 0.24 mmol), t-Bu XPhos (200 mg, 0.472 mmol) and cesium carbonate (1.54 g, 4.72 mmol) in 1,4-dioxane (4 mL)/methanol (8 mL) was stirred at 85° C. under N₂ for 10 hours. The mixture was diluted with EtOAc, washed with brine, dried over Na₂SO₄ and concentrated. The residue was purified by column chromatography on silica gel (petroleum ether:EtOAc=2:1) to give 5-methoxy-7-(1-methylindazol-5-yl)thieno[2,3-c]pyridine (380 mg, 55% yield). LCMS ESI (+) m/z 296.1 (M+H).

Step C: Preparation of 4-bromo-5-methoxy-7-(1-methylindazol-5-yl)thieno[2,3-c]pyridine: To a stirred solution of 5-methoxy-7-(1-methylindazol-5-yl)thieno[2,3-c]pyridine (380 mg, 1.29 mmol) in DMF (2 mL) at 0° C. was added NBS (275 mg, 1.54 mmol). The mixture was stirred at 0° C. for 30 min. The reaction was quenched by water and extracted with EtOAc. The combined organics were dried over Na₂SO₄ and concentrated. The residue was purified by column chromatography on silica gel (petroleum ether:EtOAc=2:1) to give 4-bromo-5-methoxy-7-(1-methylindazol-5-yl)thieno[2,3-c]pyridine (440 mg, 91% yield). LCMS ESI (+) m/z 374.0 (M+H).

Step D: Preparation of 4-[2,4-difluoro-6-(2-methoxyethoxy)phenyl]-5-methoxy-7-(1-methylindazol-5-yl)thieno[2,3-c]pyridine: A solution of 4-bromo-5-methoxy-7-(1-methylindazol-5-yl)thieno[2,3-c]pyridine (100 mg, 0.267 mmol), 2-[2,4-difluoro-6-(2-methoxyethoxy)phenyl]-5,5-dimethyl-1,3,2-dioxaborinane (241 mg, 0.802 mmol), Pd(DPEphos)Cl₂ (19 mg, 0.027 mmol) and Cs₂CO₃ (174 mg, 0.534 mmol) in 1,4-dioxane (6 mL) was stirred at 95° C. under N₂ for 12 hours. The mixture was concentrated and purified by preparative TLC (petroleum ether:EtOAc=2:1) to give 4-[2,4-difluoro-6-(2-methoxyethoxy)phenyl]-5-methoxy-7-(1-methylindazol-5-yl)thieno[2,3-c]pyridine (52 mg, 40% yield). LCMS ESI (+) m/z 482.2 (M+H).

Step E: Preparation of 4-[2,4-difluoro-6-(2-methoxyethoxy)phenyl]-7-(1-methylindazol-5-yl)thieno[2,3-c]pyridin-5-ol: A solution of 4-[2,4-difluoro-6-(2-methoxyethoxy)phenyl]-5-methoxy-7-(1-methylindazol-5-yl)thieno[2,3-c]pyridine (47 mg, 0.098 mmol), trimethylchlorosilane (0.12 mL, 0.98 mmol) and sodium iodide (44 mg, 0.29 mmol) in acetonitrile (6 mL) was stirred at 80° C. for 1 hour. The mixture was cooled to rt. Aqueous Na₂SO₃ solution was added to quench the reaction. The mixture was extracted with EtOAc, washed by brine, dried over Na₂SO₄ and concentrated to give crude 4-[2,4-difluoro-6-(2-methoxyethoxy)phenyl]-7-(1-methylindazol-5-yl)thieno[2,3-c]pyridin-5-ol (53 mg) which was used in the next step without further purification. LCMS ESI (+) m/z 468.2 (M+H).

Step F: Preparation of [4-[2,4-difluoro-6-(2-methoxyethoxy)phenyl]-7-(1-methylindazol-5-yl)thieno[2,3-c]pyridin-5-yl]trifluoromethanesulfonate: To a stirred solution of 4-[2,4-difluoro-6-(2-methoxyethoxy)phenyl]-7-(1-methylindazol-5-yl)thieno[2,3-c]pyridin-5-ol (48 mg, 0.10 mmol) and triethylamine (0.057 mL, 0.41 mmol) in DCM (2 mL) was added trifluoromethanesulfonic anhydride (0.035 mL, 0.21 mmol) at 0° C. The mixture was stirred at 5° C. for 30 min. Saturated aqueous NaHCO₃ solution was added and the reaction was stirred for additional 5 min. The mixture was extracted with EtOAc, washed with brine, dried over Na₂SO₄ and concentrated. The residue was purified by preparative TLC (petroleum ether/EtOAc=1/1) to give [4-[2,4-difluoro-6-(2-methoxyethoxy)phenyl]-7-(1-methylindazol-5-yl)thieno[2,3-c]pyridin-5-yl]trifluoromethanesulfonate (40 mg, 65% yield). LCMS ESI (+) m/z 600.2 (M+H).

Step G: Preparation of tert-butyl 3-[4-[2,4-difluoro-6-(2-methoxyethoxy)phenyl]-7-(1-methylindazol-5-yl)thieno[2,3-c]pyridin-5-yl]-7,8-dihydro-5H-1,6-naphthyridine-6-carboxylate: A solution of [4-[2,4-difluoro-6-(2-methoxyethoxy)phenyl]-7-(1-methylindazol-5-yl)thieno[2,3-c]pyridin-5-yl]trifluoromethanesulfonate (20 mg, 0.033 mmol), (6-tert-butoxycarbonyl-7,8-dihydro-5H-1,6-naphthyridin-3-yl)boronic acid (14 mg, 0.050 mmol), sodium carbonate (7.1 mg, 0.067 mmol) and tetrakis(triphenylphosphine)palladium(0) (3.9 mg, 0.0033 mmol) in 1,4-dioxane (1.2 mL) and water (0.3 mL) was stirred for 2 h at 100° C. under Ar atmosphere. The mixture was concentrated and purified by preparative TLC (MeOH/DCM=1/15) to give tert-butyl 3-[4-[2,4-difluoro-6-(2-methoxyethoxy)phenyl]-7-(1-methylindazol-5-yl)thieno[2,3-c]pyridin-5-yl]-7,8-dihydro-5H-1,6-naphthyridine-6-carboxylate (17 mg, 75% yield) as a white solid. LCMS ESI (+) m/z 684.4 (M+H).

Step H: Preparation of 4-[2,4-difluoro-6-(2-methoxyethoxy)phenyl]-7-(1-methylindazol-5-yl)-5-(5,6,7,8-tetrahydro-1,6-naphthyridin-3-yl)thieno[2,3-c]pyridine: To a solution of tert-butyl 3-[4-[2,4-difluoro-6-(2-methoxyethoxy)phenyl]-7-(1-methylindazol-5-yl)thieno[2,3-c]pyridin-5-yl]-7,8-dihydro-5H-1,6-naphthyridine-6-carboxylate (17 mg, 0.025 mmol) in DCM (3 mL) was added trifluoroacetic acid (1.0 mL). The mixture was stirred at rt for 1 h. The mixture was concentrated under reduced pressure to give crude 4-[2,4-difluoro-6-(2-methoxyethoxy)phenyl]-7-(1-methylindazol-5-yl)-5-(5,6,7,8-tetrahydro-1,6-naphthyridin-3-yl)thieno[2,3-c]pyridine (24 mg, 100% yield), which was used in the next step without further purification. LCMS ESI (+) m/z 584.3 (M+H).

Step I: Preparation of 1-[3-[4-[2,4-difluoro-6-(2-methoxyethoxy)phenyl]-7-(1-methylindazol-5-yl)thieno[2,3-c]pyridin-5-yl]-7,8-dihydro-5H-1,6-naphthyridin-6-yl]prop-2-en-1-one: To a solution of crude 4-[2,4-difluoro-6-(2-methoxyethoxy)phenyl]-7-(1-methylindazol-5-yl)-5-(5,6,7,8-tetrahydro-1,6-naphthyridin-3-yl)thieno[2,3-c]pyridine (24 mg, 0.025 mmol) in ethyl acetate (5 mL) and water (5 mL) was added sodium bicarbonate to adjust the pH to 8. Additional NaHCO₃ (4.2 mg, 0.050 mmol) was added. The mixture was cooled to at 0° C. and acryloyl chloride (2.7 mg, 0.030 mmol) in DCM (3 mL) was added. The mixture was stirred at 0° C. for 1 h. The mixture was diluted with water and extracted with EtOAc. The combined organic layers were dried over Na₂SO₄, filtered and concentrated under reduced pressure. The residue was purified by preparative HPLC to give 1-[3-[4-[2,4-difluoro-6-(2-methoxyethoxy)phenyl]-7-(1-methylindazol-5-yl)thieno[2,3-c]pyridin-5-yl]-7,8-dihydro-5H-1,6-naphthyridin-6-yl]prop-2-en-1-one (7.4 mg, 44% yield) as a bis trifluoracetic acid salt. ¹H NMR (400 MHz, CD₃OD) δ 8.61 (d, J=4.1 Hz, 2H), 8.33 (s, 1H), 8.25 (dd, J=1.6, 8.8 Hz, 1H), 8.20 (s, 1H), 8.11 (d, J=5.5 Hz, 1H), 7.80 (d, J=8.8 Hz, 1H), 7.20 (dd, J=0.9, 5.5 Hz, 1H), 6.88 (d, J=11.0 Hz, 2H), 6.73 (td, J=2.2, 9.2 Hz, 1H), 6.29 (d, J=16.5 Hz, 1H), 5.83 (d, J=10.7 Hz, 1H), 4.17 (s, 3H), 4.02-4.11 (m, 4H), 3.49 (t, J=5.0 Hz, 2H), 3.10-3.21 (m, 5H). LCMS ESI (+) m/z 638.4 (M+H).

### Biological Assays

### Biological Example 1: Disrupting KRAS-PI3Kα Interaction by SPR Inhibition Assay

SPR binding experiments were collected on a Biacore 8K Instrument (Cytiva). Neutravidin (Pierce) was amine coupled to the carboxymethylated dextran surface of a CM5 sensor chip (Cytiva) using standard amine coupling chemistry. The CM5 chip surface was first activated with 0.1 M N-hydroxy succinimide and 0.4 M N-ethyl-N′-(3-dimethyl aminopropyl) carbodiimide at a flow rate of 20 μL/min using 20 mM HEPES pH 7.4, 150 mM NaCl as the running buffer. Next, neutravidin was diluted to 20 μg/mL in 10 mM sodium acetate (pH 4.5) and injected on all flow cells until a density of approximately 10,000 response units (RU) was immobilized. Activated amine groups were quenched with an injection of 1 M ethanolamine (pH 8.0). 300-500 RU of avi-tagged full length PI3Kα was captured on all flow cells in 20 mM HEPES pH 7.4, 150 mM NaCl, 5 mM MgCl₂, 1 mM TCEP, 0.05% tween 20, 5% DMSO buffer. KRAS-Q25A at 20 μM was mixed with 8 concentrations of compound (50 nM-100 μM) and injected over the full-length PI3Kα at 30 μL/min and 25° C. Steady-state levels of KRAS binding were recorded and fit with a 4-parameter inhibition model to determine the IC₅₀ values.

For IC₅₀ values shown in Table 4, “A” means IC₅₀<5 μM; “B” means IC₅₀≥5 μM and ≤25 μM; “C” means IC₅₀>25 μM.

TABLE 4
SPR inhibition IC₅₀ of selected compounds
of the present disclosure.

| Compound No. | SPR inhibition IC₅₀ |
|---|---|
| 1 | B |
| 2 | C |
| 3 | C |
| 4 | B |
| 5 | C |
| 6 | B |
| 7 | B |
| 8 | C |
| 9 | B |
| 10 | A |
| 11 | A |
| 12 | A |
| 13 | B |
| 14 | B |
| 15 | A |
| 16 | B |
| 17 | B |
| 18 | A |
| 19 | A |
| 20 | C |
| 21 | B |
| 22 | B |
| 23 | C |
| 24 | A |
| 25 | B |
| 26 | A |
| 27 | A |
| 28 | B |
| 29 | A |
| 30 | B |
| 31 | A |
| 32 | B |
| 33 | A |
| 34 | B |
| 35 | B |
| 36 | A |
| 37 | B |
| 38 | B |
| 39 | B |
| 40 | A |
| 41 | B |
| 42 | A |
| 43 | B |
| 44 | B |
| 45 | B |
| 46 | B |
| 47 | B |
| 48 | B |
| 49 | C |
| 50 | B |
| 51 | B |
| 52 | B |
| 53 | B |
| 54 | B |
| 55 | B |
| 56 | B |
| 57 | C |
| 58 | B |
| 59 | A |
| 60 | B |
| 229 | A |

### Biological Example 2: Covalent Modification Assay Using MALDI-TOF MS

Matrix-Assisted Laser Desorption Ionization-Time of Flight Mass Spectrometry (MALDI-TOF MS) analysis of covalent modification of Cys242 in human PIK3CA (157-299) was performed. Initially 3.33 μM protein target and 1:7.5 protein to compound ratio were used; later the target protein concentration was adjusted to 1 μM with 1:5 protein to compound ratio for more potent compounds.

Reaction: 3.33 μM or 1 μM of PIK3CA (157-299) protein (produced in-house by Protein Expression Laboratory, FNLCR/Leidos Biomed) in 20 mM HEPES buffer containing 150 mM NaCl, 2 mM MgCl₂, pH 7.3 was prepared freshly before assay. Twelve-μL aliquots of protein were dispensed onto low volume 384-well plate, then 470 nL DMSO and 30 nL of tested compounds from 10 mM DMSO stocks were added to appropriate wells using ECHO 555 acoustic liquid handler (Labcyte Inc.). For each reaction/assay, three blanks were prepared by mixing 12 μL of protein solution with 500 nL DMSO. The wells content was carefully mixed by aspiration, and then the plate was sealed by an adhesive cover, centrifuged at 931 g for 1 minute, and kept in the dark at room temperature until 15 min, 30 min, 2 h, or 4 h collections.

Targetpretreatment: Before each assay MALDI target (Bruker MPT 384 ground steel BC) was pre-treated by pipetting on each spot 0.75 μL of saturated sinapinic acid in acetonitrile (ACN). This step significantly improves the uniformity of sample crystallization across the plate resulting in enhanced sensitivity.

Sample preparation: At collection time point, 2 μL of reaction mixtures were pipetted out into L MALDI matrix solution (saturated solution of sinapinic acid in 1:1 ACN:water solution containing 0.15% trifluoroacetic acid (TFA)) deposited on 384 well polypropylene plate. The resulting solution was mixed by aspiration, centrifuged at 931 g for 1 minute, then 1.5 μL aliquots were dispensed on pre-treated MALDI target using Beckman Coulter Biomek FX^{P} 96/384-Span-8 Laboratory Automation Workstation. Finally, the MALDI target was dried under a mild vacuum to produce spots with a fine crystalline structure.

Measurements: MALDI-TOF measurements were performed on Bruker Daltonics rapifleX Tissuetyper TOF-TOF mass spectrometer using linear mode and mass ranging from 16.0 to 19.6 kDa. Detector gain was set to 0.64×(459 V), sample rate to 5 GS/s, real-time smoothing to medium (175 MHz), laser smart beam pattern was set to “Custom” single smartbeam beam scan with a scan range of 40 μm on both X and Y axis, and the laser frequency was 10000 Hz. Spectra were automatically collected using the custom AutoXecute method. Laser power was auto adjusted using fuzzy control. The peak selection range was set to be between 16.0 and 18.5 kDa. Peak evaluation uses a half-width parameter set to be smaller than 40 Da for processed spectrograms (centroid peak detection; smoothed by SavitzkyGolay algorithm using 7 m/z width and 2 cycles; baseline was subtracted using a median algorithm with flatness 1 and median level 0). Fuzzy control used Proteins/Oligonucleotides protocol with minimum half-width 1/10 times above threshold. Up to 40000 satisfactory shots were collected in 10000 short steps. Dynamic termination was implemented to finish data collection when the peak signal/noise ratio was reaching a value of 1000.

Spectra processing: Spectra were smoothed by SavitzkyGolay algorithm using 7 m/z width and three cycles. Centroid peak detection algorithm was used with signal to noise threshold set to 6, relative intensity threshold 3%, peak width 10 m/z, and median baseline subtraction using flatness of 1 and the median level of 0.1. Peak intensity and area under the peak were evaluated and recorded for all peaks between 16.0 and 19.5 kDa.

Calculation of percent modification: Percent modification was calculated as a ratio of peak height for protein modified by compound to the sum of the peak height of remaining protein plus peak height for protein modified by a compound. If multiple modifications were observed each was calculated as a ratio of peak height for given modification versus the sum of peak heights for all observed protein species.

Data reported in Table 5 are from 4 h reaction time point. “A” means percent of modification of PIK3CA protein ≥75%; “B” means 50%<percent of modification <75%; “C” means 25%≤percent of modification <50%; “D” means percent of modification <25%. In Table 5, “Cmd” means “Compound” and “% mod” means “% of modification.” Data for compounds 1-852 were collected using 3.33 μM protein target and 1:7.5 protein to compound ratio; data for compounds 853-1279 were collected using 1 μM protein target with 1:5 protein to compound ratio.

TABLE 5
Covalent modification using MALDI-TOF MS of
selected compounds of the present disclosure.

| Cmd No. | % mod |
|---|---|
| 1 | D |
| 11 | C |
| 12 | A |
| 22 | B |
| 23 | A |
| 28 | A |
| 30 | A |
| 31 | A |
| 32 | A |
| 35 | D |
| 36 | A |
| 38 | B |
| 40 | A |
| 42 | A |
| 43 | A |
| 44 | A |
| 46 | A |
| 47 | A |
| 49 | A |
| 50 | A |
| 51 | A |
| 52 | A |
| 53 | A |
| 54 | A |
| 55 | A |
| 56 | B |
| 57 | B |
| 58 | A |
| 59 | A |
| 60 | A |
| 61 | A |
| 62 | A |
| 63 | A |
| 64 | A |
| 65 | D |
| 66 | D |
| 67 | A |
| 68 | A |
| 69 | A |
| 70 | A |
| 71 | A |
| 72 | A |
| 73 | A |
| 74 | D |
| 75 | B |
| 76 | A |
| 77 | D |
| 78 | A |
| 79 | A |
| 80 | A |
| 81 | D |
| 82 | A |
| 83 | A |
| 84 | B |
| 85 | A |
| 86 | A |
| 87 | A |
| 88 | A |
| 89 | A |
| 90 | D |
| 91 | D |
| 92 | A |
| 93 | C |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | C |
| 107 | A |
| 108 | A |
| 109 | A |
| 110 | A |
| 111 | A |
| 112 | A |
| 113 | D |
| 114 | A |
| 115 | A |
| 116 | A |
| 117 | A |
| 118 | A |
| 119 | A |
| 120 | A |
| 121 | A |
| 122 | A |
| 123 | A |
| 125 | A |
| 126 | A |
| 127 | D |
| 128 | A |
| 129 | D |
| 130 | A |
| 131 | A |
| 132 | A |
| 133 | A |
| 134 | B |
| 135 | A |
| 136 | A |
| 137 | C |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | B |
| 160 | A |
| 161 | A |
| 162 | A |
| 163 | A |
| 164 | A |
| 165 | A |
| 166 | A |
| 167 | B |
| 168 | A |
| 169 | B |
| 170 | D |
| 171 | D |
| 172 | A |
| 173 | A |
| 174 | A |
| 175 | A |
| 176 | A |
| 177 | B |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | B |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 230 | B |
| 232 | A |
| 233 | A |
| 234 | A |
| 235 | A |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |
| 257 | A |
| 258 | B |
| 259 | A |
| 261 | A |
| 262 | A |
| 263 | A |
| 264 | A |
| 265 | A |
| 266 | A |
| 267 | A |
| 268 | A |
| 270 | A |
| 271 | A |
| 272 | A |
| 273 | C |
| 274 | A |
| 275 | A |
| 276 | A |
| 277 | A |
| 278 | B |
| 279 | A |
| 280 | A |
| 281 | C |
| 282 | A |
| 283 | A |
| 284 | A |
| 285 | B |
| 286 | A |
| 287 | A |
| 288 | A |
| 289 | A |
| 291 | A |
| 292 | A |
| 293 | A |
| 294 | A |
| 295 | B |
| 296 | B |
| 297 | B |
| 298 | A |
| 299 | A |
| 300 | A |
| 301 | A |
| 302 | A |
| 303 | A |
| 304 | B |
| 305 | B |
| 306 | B |
| 307 | A |
| 308 | A |
| 309 | A |
| 310 | A |
| 311 | A |
| 312 | A |
| 313 | A |
| 314 | A |
| 315 | A |
| 316 | A |
| 317 | A |
| 318 | A |
| 319 | A |
| 320 | A |
| 321 | B |
| 322 | C |
| 323 | A |
| 324 | A |
| 325 | B |
| 326 | A |
| 327 | A |
| 328 | A |
| 329 | A |
| 330 | A |
| 331 | A |
| 332 | A |
| 333 | A |
| 334 | A |
| 335 | A |
| 336 | A |
| 337 | B |
| 338 | B |
| 339 | A |
| 340 | B |
| 341 | B |
| 342 | B |
| 343 | A |
| 344 | A |
| 345 | A |
| 346 | A |
| 347 | A |
| 348 | A |
| 349 | A |
| 350 | A |
| 351 | A |
| 352 | A |
| 353 | B |
| 354 | B |
| 355 | A |
| 356 | A |
| 357 | A |
| 358 | A |
| 359 | A |
| 360 | B |
| 361 | A |
| 362 | A |
| 363 | A |
| 364 | A |
| 365 | A |
| 366 | A |
| 367 | A |
| 368 | A |
| 369 | A |
| 370 | A |
| 371 | A |
| 372 | A |
| 373 | A |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 385 | A |
| 386 | A |
| 387 | A |
| 388 | A |
| 389 | A |
| 390 | A |
| 391 | A |
| 392 | A |
| 393 | A |
| 394 | A |
| 395 | A |
| 396 | A |
| 397 | A |
| 398 | A |
| 399 | A |
| 400 | A |
| 401 | A |
| 402 | A |
| 403 | A |
| 404 | A |
| 405 | A |
| 406 | A |
| 407 | A |
| 408 | A |
| 409 | A |
| 410 | A |
| 411 | A |
| 412 | A |
| 413 | D |
| 414 | A |
| 415 | A |
| 416 | A |
| 417 | A |
| 418 | A |
| 419 | A |
| 420 | A |
| 421 | A |
| 422 | A |
| 423 | A |
| 424 | A |
| 425 | A |
| 426 | A |
| 427 | A |
| 428 | A |
| 429 | A |
| 430 | A |
| 431 | A |
| 432 | A |
| 433 | A |
| 434 | A |
| 435 | A |
| 436 | A |
| 437 | A |
| 438 | A |
| 439 | D |
| 440 | D |
| 441 | A |
| 442 | A |
| 443 | A |
| 444 | A |
| 445 | A |
| 446 | A |
| 447 | D |
| 448 | A |
| 449 | D |
| 450 | A |
| 451 | A |
| 452 | A |
| 453 | A |
| 454 | A |
| 455 | A |
| 456 | D |
| 457 | A |
| 458 | A |
| 459 | A |
| 461 | A |
| 462 | A |
| 463 | A |
| 464 | A |
| 465 | A |
| 466 | A |
| 467 | A |
| 468 | A |
| 469 | A |
| 470 | A |
| 471 | A |
| 472 | B |
| 473 | D |
| 474 | A |
| 475 | B |
| 476 | A |
| 477 | B |
| 478 | D |
| 479 | A |
| 480 | A |
| 481 | A |
| 482 | A |
| 483 | A |
| 484 | A |
| 485 | A |
| 486 | A |
| 487 | D |
| 488 | A |
| 489 | A |
| 490 | A |
| 491 | A |
| 492 | A |
| 493 | A |
| 494 | A |
| 495 | A |
| 496 | A |
| 497 | A |
| 498 | A |
| 499 | A |
| 500 | D |
| 501 | D |
| 502 | A |
| 503 | A |
| 504 | A |
| 505 | A |
| 506 | A |
| 507 | A |
| 508 | A |
| 509 | A |
| 510 | A |
| 511 | A |
| 512 | A |
| 513 | A |
| 514 | A |
| 515 | A |
| 516 | A |
| 517 | B |
| 518 | A |
| 519 | A |
| 520 | A |
| 521 | A |
| 522 | A |
| 523 | A |
| 524 | A |
| 525 | A |
| 526 | A |
| 527 | B |
| 528 | A |
| 529 | A |
| 530 | A |
| 531 | D |
| 532 | A |
| 533 | A |
| 534 | A |
| 535 | A |
| 536 | A |
| 537 | A |
| 538 | A |
| 539 | A |
| 544 | A |
| 545 | A |
| 546 | A |
| 547 | A |
| 548 | A |
| 549 | A |
| 550 | A |
| 551 | A |
| 552 | A |
| 553 | A |
| 554 | A |
| 555 | D |
| 556 | A |
| 557 | D |
| 558 | D |
| 559 | A |
| 560 | A |
| 561 | A |
| 562 | C |
| 563 | A |
| 564 | D |
| 565 | A |
| 566 | D |
| 567 | A |
| 568 | A |
| 569 | A |
| 570 | D |
| 571 | D |
| 572 | A |
| 573 | A |
| 574 | A |
| 575 | D |
| 576 | A |
| 577 | A |
| 578 | D |
| 579 | D |
| 580 | A |
| 581 | D |
| 582 | A |
| 583 | A |
| 584 | A |
| 585 | A |
| 586 | D |
| 587 | D |
| 588 | A |
| 589 | D |
| 590 | A |
| 591 | D |
| 592 | A |
| 593 | D |
| 594 | A |
| 595 | A |
| 596 | A |
| 597 | D |
| 598 | A |
| 599 | D |
| 600 | A |
| 601 | A |
| 602 | A |
| 603 | D |
| 604 | A |
| 605 | A |
| 606 | D |
| 607 | A |
| 608 | D |
| 609 | A |
| 610 | A |
| 611 | D |
| 612 | A |
| 613 | D |
| 614 | A |
| 615 | D |
| 616 | A |
| 617 | A |
| 618 | A |
| 619 | D |
| 620 | A |
| 621 | A |
| 622 | A |
| 623 | A |
| 624 | A |
| 625 | A |
| 626 | D |
| 627 | A |
| 628 | A |
| 629 | D |
| 630 | D |
| 631 | A |
| 632 | D |
| 633 | D |
| 634 | A |
| 635 | A |
| 636 | A |
| 637 | A |
| 638 | A |
| 639 | A |
| 640 | A |
| 641 | C |
| 642 | A |
| 643 | A |
| 644 | A |
| 645 | A |
| 646 | A |
| 647 | A |
| 648 | D |
| 649 | A |
| 650 | C |
| 651 | C |
| 652 | C |
| 653 | B |
| 654 | A |
| 655 | A |
| 656 | C |
| 657 | D |
| 658 | A |
| 659 | C |
| 660 | C |
| 661 | C |
| 662 | A |
| 663 | A |
| 664 | A |
| 665 | A |
| 666 | A |
| 667 | A |
| 668 | A |
| 669 | A |
| 670 | A |
| 671 | A |
| 672 | A |
| 673 | D |
| 674 | A |
| 676 | A |
| 677 | A |
| 678 | A |
| 679 | A |
| 680 | A |
| 681 | A |
| 682 | A |
| 683 | A |
| 684 | A |
| 685 | A |
| 686 | A |
| 687 | A |
| 688 | A |
| 689 | A |
| 690 | A |
| 691 | A |
| 692 | A |
| 693 | A |
| 694 | A |
| 695 | A |
| 696 | A |
| 697 | A |
| 698 | A |
| 699 | A |
| 700 | A |
| 701 | A |
| 702 | A |
| 703 | A |
| 704 | A |
| 705 | A |
| 706 | A |
| 708 | A |
| 709 | A |
| 710 | A |
| 711 | A |
| 712 | A |
| 713 | A |
| 714 | A |
| 715 | A |
| 716 | A |
| 717 | A |
| 718 | A |
| 719 | C |
| 720 | A |
| 721 | A |
| 722 | A |
| 723 | A |
| 724 | A |
| 725 | A |
| 726 | A |
| 727 | A |
| 728 | D |
| 729 | A |
| 730 | A |
| 731 | A |
| 732 | A |
| 733 | A |
| 734 | A |
| 735 | A |
| 736 | A |
| 737 | A |
| 738 | A |
| 739 | A |
| 740 | A |
| 741 | A |
| 742 | A |
| 743 | A |
| 744 | A |
| 745 | A |
| 746 | A |
| 747 | A |
| 748 | A |
| 749 | A |
| 750 | A |
| 751 | A |
| 752 | A |
| 753 | A |
| 754 | A |
| 755 | A |
| 756 | A |
| 757 | A |
| 758 | A |
| 759 | A |
| 760 | A |
| 761 | A |
| 762 | A |
| 763 | A |
| 764 | A |
| 765 | A |
| 766 | A |
| 767 | A |
| 768 | A |
| 769 | A |
| 770 | A |
| 771 | A |
| 772 | A |
| 773 | A |
| 774 | A |
| 775 | A |
| 776 | A |
| 777 | A |
| 778 | A |
| 779 | A |
| 780 | A |
| 781 | A |
| 782 | D |
| 783 | A |
| 784 | D |
| 785 | A |
| 786 | A |
| 787 | D |
| 788 | A |
| 789 | A |
| 790 | A |
| 791 | A |
| 792 | A |
| 793 | A |
| 794 | A |
| 795 | A |
| 796 | A |
| 797 | A |
| 798 | A |
| 799 | A |
| 800 | A |
| 801 | A |
| 802 | A |
| 803 | A |
| 804 | A |
| 805 | A |
| 806 | A |
| 807 | A |
| 808 | A |
| 809 | A |
| 810 | D |
| 811 | A |
| 812 | A |
| 813 | A |
| 814 | A |
| 815 | A |
| 816 | A |
| 817 | A |
| 818 | A |
| 819 | A |
| 820 | A |
| 821 | A |
| 822 | A |
| 823 | A |
| 824 | A |
| 825 | A |
| 826 | A |
| 827 | A |
| 828 | A |
| 829 | A |
| 830 | A |
| 831 | A |
| 832 | A |
| 833 | A |
| 834 | A |
| 835 | A |
| 836 | A |
| 837 | A |
| 838 | A |
| 839 | A |
| 840 | A |
| 841 | A |
| 842 | A |
| 843 | A |
| 844 | A |
| 845 | A |
| 846 | A |
| 847 | A |
| 848 | A |
| 849 | A |
| 850 | A |
| 851 | A |
| 852 | A |
| 853 | A |
| 854 | A |
| 855 | A |
| 856 | A |
| 857 | A |
| 858 | A |
| 859 | A |
| 860 | A |
| 861 | A |
| 862 | A |
| 863 | A |
| 864 | A |
| 865 | D |
| 866 | A |
| 867 | A |
| 868 | A |
| 869 | A |
| 870 | A |
| 871 | A |
| 872 | A |
| 873 | D |
| 874 | A |
| 875 | A |
| 876 | A |
| 877 | A |
| 878 | A |
| 879 | A |
| 880 | A |
| 881 | A |
| 882 | A |
| 883 | A |
| 884 | A |
| 885 | A |
| 886 | D |
| 887 | A |
| 888 | A |
| 889 | A |
| 890 | A |
| 891 | A |
| 892 | A |
| 893 | A |
| 894 | A |
| 895 | A |
| 896 | A |
| 897 | A |
| 898 | A |
| 899 | A |
| 900 | A |
| 901 | A |
| 902 | A |
| 903 | A |
| 904 | A |
| 905 | C |
| 906 | A |
| 907 | A |
| 908 | A |
| 909 | A |
| 910 | B |
| 911 | B |
| 912 | A |
| 913 | A |
| 914 | A |
| 915 | A |
| 916 | D |
| 917 | A |
| 918 | A |
| 919 | A |
| 920 | A |
| 921 | D |
| 922 | D |
| 923 | A |
| 924 | A |
| 925 | A |
| 926 | A |
| 927 | A |
| 928 | A |
| 929 | A |
| 930 | A |
| 931 | A |
| 932 | A |
| 933 | A |
| 934 | A |
| 935 | A |
| 936 | A |
| 937 | A |
| 938 | A |
| 939 | A |
| 940 | A |
| 941 | A |
| 942 | A |
| 943 | A |
| 944 | A |
| 945 | A |
| 946 | A |
| 947 | A |
| 948 | A |
| 949 | A |
| 950 | D |
| 951 | A |
| 952 | A |
| 953 | A |
| 954 | A |
| 955 | A |
| 956 | A |
| 957 | A |
| 958 | A |
| 959 | A |
| 960 | A |
| 961 | A |
| 962 | A |
| 963 | A |
| 964 | A |
| 965 | A |
| 966 | A |
| 967 | A |
| 968 | A |
| 969 | A |
| 970 | A |
| 971 | A |
| 972 | D |
| 973 | A |
| 974 | D |
| 975 | D |
| 976 | A |
| 977 | A |
| 978 | A |
| 979 | A |
| 980 | A |
| 981 | A |
| 982 | A |
| 983 | A |
| 984 | A |
| 985 | A |
| 986 | A |
| 987 | A |
| 988 | A |
| 989 | A |
| 990 | A |
| 991 | A |
| 992 | A |
| 993 | A |
| 994 | D |
| 995 | A |
| 996 | A |
| 997 | A |
| 998 | A |
| 999 | A |
| 1000 | A |
| 1001 | D |
| 1002 | A |
| 1003 | A |
| 1005 | D |
| 1006 | D |
| 1007 | A |
| 1008 | A |
| 1009 | A |
| 1011 | A |
| 1012 | A |
| 1013 | A |
| 1014 | A |
| 1015 | A |
| 1016 | A |
| 1017 | A |
| 1018 | A |
| 1019 | A |
| 1020 | A |
| 1021 | A |
| 1022 | A |
| 1023 | A |
| 1024 | A |
| 1025 | A |
| 1026 | A |
| 1027 | A |
| 1028 | A |
| 1029 | A |
| 1030 | A |
| 1031 | B |
| 1032 | D |
| 1033 | D |
| 1035 | A |
| 1036 | A |
| 1037 | A |
| 1038 | A |
| 1039 | A |
| 1040 | A |
| 1041 | A |
| 1042 | A |
| 1043 | A |
| 1044 | A |
| 1045 | A |
| 1046 | A |
| 1047 | A |
| 1048 | A |
| 1049 | A |
| 1050 | A |
| 1051 | A |
| 1052 | A |
| 1053 | A |
| 1054 | C |
| 1055 | C |
| 1056 | A |
| 1057 | A |
| 1058 | A |
| 1059 | A |
| 1060 | A |
| 1061 | A |
| 1062 | A |
| 1063 | A |
| 1064 | C |
| 1065 | A |
| 1066 | D |
| 1067 | D |
| 1068 | D |
| 1069 | D |
| 1070 | A |
| 1071 | B |
| 1072 | A |
| 1073 | A |
| 1074 | A |
| 1076 | A |
| 1077 | A |
| 1078 | A |
| 1079 | A |
| 1080 | A |
| 1081 | A |
| 1082 | A |
| 1083 | A |
| 1084 | A |
| 1085 | A |
| 1086 | A |
| 1087 | A |
| 1088 | B |
| 1089 | A |
| 1090 | A |
| 1091 | A |
| 1092 | A |
| 1093 | A |
| 1094 | A |
| 1095 | A |
| 1096 | A |
| 1097 | A |
| 1099 | A |
| 1100 | A |
| 1101 | A |
| 1102 | A |
| 1103 | A |
| 1104 | A |
| 1105 | A |
| 1106 | A |
| 1107 | A |
| 1108 | A |
| 1109 | A |
| 1111 | C |
| 1112 | A |
| 1113 | A |
| 1114 | A |
| 1115 | A |
| 1117 | A |
| 1118 | A |
| 1119 | A |
| 1120 | A |
| 1122 | A |
| 1123 | A |
| 1124 | A |
| 1125 | A |
| 1126 | A |
| 1128 | A |
| 1129 | A |
| 1130 | A |
| 1131 | D |
| 1132 | A |
| 1133 | A |
| 1134 | A |
| 1135 | A |
| 1136 | A |
| 1137 | A |
| 1138 | A |
| 1139 | A |
| 1140 | A |
| 1141 | A |
| 1142 | A |
| 1144 | A |
| 1145 | A |
| 1146 | A |
| 1147 | A |
| 1148 | A |
| 1149 | A |
| 1150 | A |
| 1151 | A |
| 1152 | A |
| 1153 | A |
| 1154 | A |
| 1155 | A |
| 1156 | D |
| 1157 | A |
| 1158 | A |
| 1159 | A |
| 1160 | A |
| 1161 | A |
| 1162 | A |
| 1163 | A |
| 1164 | A |
| 1165 | A |
| 1166 | D |
| 1167 | A |
| 1168 | B |
| 1169 | A |
| 1170 | A |
| 1171 | A |
| 1172 | A |
| 1173 | C |
| 1174 | A |
| 1175 | A |
| 1176 | A |
| 1177 | A |
| 1178 | A |
| 1179 | A |
| 1180 | A |
| 1181 | A |
| 1182 | A |
| 1183 | A |
| 1184 | A |
| 1185 | A |
| 1186 | A |
| 1187 | A |
| 1188 | A |
| 1189 | A |
| 1190 | A |
| 1191 | A |
| 1192 | A |
| 1193 | A |
| 1194 | A |
| 1195 | A |
| 1196 | A |
| 1197 | A |
| 1198 | A |
| 1199 | A |
| 1200 | A |
| 1201 | A |
| 1202 | A |
| 1203 | A |
| 1205 | A |
| 1206 | A |
| 1207 | A |
| 1208 | A |
| 1209 | A |
| 1210 | A |
| 1211 | A |
| 1212 | A |
| 1213 | A |
| 1214 | A |
| 1215 | D |
| 1216 | A |
| 1217 | A |
| 1218 | A |
| 1219 | A |
| 1220 | A |
| 1221 | A |
| 1222 | A |
| 1223 | A |
| 1224 | A |
| 1225 | A |
| 1226 | D |
| 1227 | B |
| 1228 | D |
| 1229 | A |
| 1230 | A |
| 1231 | A |
| 1232 | A |
| 1233 | D |
| 1234 | A |
| 1237 | A |
| 1239 | C |
| 1240 | A |
| 1242 | A |
| 1243 | A |
| 1244 | A |
| 1245 | A |
| 1246 | B |
| 1248 | A |
| 1249 | A |
| 1250 | A |
| 1251 | A |
| 1252 | A |
| 1253 | A |
| 1254 | A |
| 1255 | A |
| 1256 | D |
| 1257 | A |
| 1258 | A |
| 1259 | A |
| 1260 | A |
| 1261 | A |
| 1262 | A |
| 1263 | A |
| 1264 | D |
| 1265 | A |
| 1266 | D |
| 1267 | A |
| 1269 | A |
| 1270 | A |
| 1271 | D |
| 1272 | D |
| 1273 | D |
| 1274 | A |
| 1275 | A |
| 1276 | A |
| 1277 | D |
| 1278 | A |
| 1279 | A |

### Biological Example 3: Matrix-Assisted Cell-Based pAKT HTRF Assay in Tet-on KRAS G12D HeLa Cells

To determine the effectiveness of compounds in disrupting PIK3CA/KRAS signaling in cells, we implemented pAKT HTRF assay (Perkin-Elmer) in Tet-on KRAS G12D HeLa cells.

On Day 1, cells were seeded into 96-well plates at 2×104 cells/well in complete growth media (DMEM, 10% Tet-Screened FBS) supplemented with 200 ng/mL doxycycline.

On Day 2, cells were treated with compounds at 0.25% DMSO. The source plate was created with compounds diluted in media at 5-fold the final assay concentration. The compounds are run in a 9-point concentration curve starting at 75 μM, with a 3-fold dilution between concentrations. As the potency increased, the highest dose was decreased to 25 μM while keeping the 3-fold serial dilution. 20 μL was transferred onto the cell plates (final volume in wells was 100 μL). Plates were harvested after 30 min and 4 hr incubation by aspirating media and adding kit-supplied 1× supplemented lysis buffer to all wells (50 μL per well). This then changed to 4 hr only when compounds became covalent. Plates were then placed on a plate shaker and incubated at 850 rpm for an additional 30 min. The antibody mixture solution was prepared by diluting aliquoted d2 and Eu Cryptate antibodies 1:20 in kit supplied detection buffer, then mixed the diluted antibodies solutions (1:1 v:v). 4 μL of this solution was then added to a 384-well detection plate (Perkin Elmer; 6008230). Samples were homogenized by pipetting up and down and then transferred (16 μL of cell lysates) from the 96-well cell culture plate to two wells of the HTRF 384-well detection plate containing the antibody solution. Plates were centrifuged (524 g for 1 min) and allowed to incubate between 4 and 24 h at room temperature. The maximum signal is reached after 4 h incubation time and remains stable over a period of 24 hours. Therefore, readings can be made between 4 and 24 h of incubation. Plates were centrifuged again (524 g for 1 min), and analyzed on the EnVision plate reader using the following settings: Excitation 320 nM, Bandwidth 75 nM; Emission 615 nM, Bandwidth 85 nM, Gain 100%, Flashes 100, Lag 60 μs.

For pAKT inhibition IC₅₀ values shown in Table 6, “A” means IC₅₀<1 μM; “B” means IC₅₀≥1 μM and ≤10 μM; “C” means IC₅₀>10 μM.

TABLE 6
pAKT inhibition IC₅₀ of selected compounds
in Tet-on KRAS G12D HeLa cells.

| | pAKT in Tet-on KRAS |
|---|---|
| Compound No. | G12D HeLa cells |
| 1 | B |
| 6 | B |
| 7 | B |
| 8 | C |
| 9 | B |
| 10 | B |
| 11 | B |
| 12 | B |
| 13 | B |
| 14 | C |
| 15 | B |
| 16 | B |
| 17 | B |
| 18 | B |
| 19 | C |
| 29 | B |
| 30 | A |
| 31 | A |
| 32 | A |
| 33 | B |
| 36 | B |
| 40 | B |
| 41 | B |
| 42 | A |
| 43 | A |
| 44 | A |
| 46 | A |
| 47 | A |
| 48 | A |
| 51 | A |

### Biological Example 4: Matrix-Assisted Cell-Based pAKT HTRF Assay in BT474 Cells

On Day 1, cells were seeded into 96-well plates at 2.5×104 cells/well in complete growth media (DMEM, 10% FBS). On Day 2, cells were treated with compounds at 0.25% DMSO. The source plate was created with compounds diluted in media at 5-fold the final assay concentration. The compounds are run in a 9-point concentration curve starting at 3 μM, with a 3-fold dilution between concentrations. 20 μL was transferred onto the cell plates (final volume in wells was 100 μL). Plates were harvested after 4 hr incubation by aspirating media and adding kit-supplied 1× supplemented lysis buffer to all wells (75 μL per well). Plates were then placed on a plate shaker and incubated at 850 rpm for an additional 30 min. The antibody mixture solution was prepared by diluting aliquoted d2 and Eu Cryptate antibodies 1:20 in kit supplied detection buffer, then mixed the diluted antibodies solutions (1:1 v:v). 4 μL of this solution was then added to a 384-well detection plate (Perkin Elmer; 6008230). Samples were homogenized by pipetting up and down and then transferred (16 μL of cell lysates) from the 96-well cell culture plate to two wells of the HTRF 384-well detection plate containing the antibody solution. Plates were centrifuged (524 g for 1 min) and allowed to incubate between 4 and 24 h at room temperature. The maximum signal is reached after 4 h incubation time and remains stable over a period of 24 hours. Therefore, readings can be made between 4 and 24 h of incubation. Plates were centrifuged again (524 g for 1 m), and analyzed on the EnVision plate reader using the following settings: Excitation 320 nM, Bandwidth 75 nM; Emission 615 nM, Bandwidth 85 nM, Gain 10000, Flashes 100, Lag 60 μs.

For pAKT inhibition IC₅₀ values shown in Table 7 for Compounds 10-999, “A” means IC₅₀<0.1 μM; “B” means IC₅₀≥0.1 μM and <1 μM; “C” means IC₅₀≥1 μM and ≤3 μM; “D” means IC₅₀>3 4 μM. For pAKT inhibition IC₅₀ values shown in Table 7 for Compounds 1000-1279, “A” means IC₅₀<0.1 μM; “B” means IC₅₀≥0.1 μM and <1 μM; “C” means IC₅₀>1 μM. In Table 7, “Cmd” means “Compound” and “pAKT” means “pAKT in BT474 cells.”

TABLE 7
pAKT inhibition IC₅₀ of selected compounds in BT474 cells.

| Cmd | |
|---|---|
| No. | pAKT |
| 10 | C |
| 12 | B |
| 23 | C |
| 28 | A |
| 31 | A |
| 42 | A |
| 44 | A |
| 47 | A |
| 49 | B |
| 50 | A |
| 51 | B |
| 52 | B |
| 53 | B |
| 54 | A |
| 55 | B |
| 58 | A |
| 59 | A |
| 60 | B |
| 61 | A |
| 62 | A |
| 64 | A |
| 67 | A |
| 68 | B |
| 69 | C |
| 70 | B |
| 71 | B |
| 72 | A |
| 73 | B |
| 74 | C |
| 75 | D |
| 76 | A |
| 77 | D |
| 78 | A |
| 79 | A |
| 80 | B |
| 82 | A |
| 83 | A |
| 84 | C |
| 85 | B |
| 86 | A |
| 87 | B |
| 88 | A |
| 89 | B |
| 90 | D |
| 91 | D |
| 92 | A |
| 93 | D |
| 94 | A |
| 95 | A |
| 96 | B |
| 97 | B |
| 98 | C |
| 99 | A |
| 100 | B |
| 101 | A |
| 102 | B |
| 103 | B |
| 104 | B |
| 105 | A |
| 106 | D |
| 107 | A |
| 108 | B |
| 109 | B |
| 110 | B |
| 111 | A |
| 112 | B |
| 113 | D |
| 114 | C |
| 115 | A |
| 116 | A |
| 117 | A |
| 118 | A |
| 119 | A |
| 120 | B |
| 121 | A |
| 122 | B |
| 123 | B |
| 124 | D |
| 125 | A |
| 126 | A |
| 127 | C |
| 128 | A |
| 129 | D |
| 130 | A |
| 131 | A |
| 132 | B |
| 133 | A |
| 134 | C |
| 135 | A |
| 136 | B |
| 137 | C |
| 138 | B |
| 139 | B |
| 140 | B |
| 141 | A |
| 142 | A |
| 143 | B |
| 144 | A |
| 145 | C |
| 146 | A |
| 147 | C |
| 148 | A |
| 149 | B |
| 150 | B |
| 151 | A |
| 152 | A |
| 153 | A |
| 154 | A |
| 155 | A |
| 156 | A |
| 157 | A |
| 158 | B |
| 159 | B |
| 160 | A |
| 161 | A |
| 162 | A |
| 163 | A |
| 164 | A |
| 165 | A |
| 166 | B |
| 167 | B |
| 168 | A |
| 169 | B |
| 170 | C |
| 171 | D |
| 172 | A |
| 173 | B |
| 174 | A |
| 175 | B |
| 176 | B |
| 177 | B |
| 178 | A |
| 179 | A |
| 180 | B |
| 181 | B |
| 182 | A |
| 183 | B |
| 184 | A |
| 185 | A |
| 186 | A |
| 187 | A |
| 188 | B |
| 189 | A |
| 190 | A |
| 191 | A |
| 192 | A |
| 193 | A |
| 194 | A |
| 195 | A |
| 196 | B |
| 197 | A |
| 198 | A |
| 199 | A |
| 200 | A |
| 201 | A |
| 202 | A |
| 203 | A |
| 204 | B |
| 205 | A |
| 206 | A |
| 207 | A |
| 208 | A |
| 209 | A |
| 210 | A |
| 211 | A |
| 212 | A |
| 213 | B |
| 214 | B |
| 215 | B |
| 216 | B |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | B |
| 229 | D |
| 230 | B |
| 231 | D |
| 232 | A |
| 233 | A |
| 234 | A |
| 235 | B |
| 236 | B |
| 237 | B |
| 238 | A |
| 239 | A |
| 240 | A |
| 241 | B |
| 242 | A |
| 243 | B |
| 244 | A |
| 245 | A |
| 246 | A |
| 247 | A |
| 248 | A |
| 249 | A |
| 250 | A |
| 251 | B |
| 252 | A |
| 253 | A |
| 254 | A |
| 255 | B |
| 256 | B |
| 257 | A |
| 258 | A |
| 259 | A |
| 260 | D |
| 261 | A |
| 262 | A |
| 263 | A |
| 264 | D |
| 265 | A |
| 266 | A |
| 267 | B |
| 268 | B |
| 269 | B |
| 270 | A |
| 271 | A |
| 272 | A |
| 273 | D |
| 274 | B |
| 275 | A |
| 276 | B |
| 277 | B |
| 278 | B |
| 279 | B |
| 280 | A |
| 281 | D |
| 282 | A |
| 283 | A |
| 284 | B |
| 285 | B |
| 286 | D |
| 287 | A |
| 288 | A |
| 289 | A |
| 291 | A |
| 292 | B |
| 293 | A |
| 294 | A |
| 295 | B |
| 296 | C |
| 297 | C |
| 298 | B |
| 299 | B |
| 300 | A |
| 301 | A |
| 302 | A |
| 303 | B |
| 304 | B |
| 305 | B |
| 306 | A |
| 307 | B |
| 308 | B |
| 309 | A |
| 310 | A |
| 311 | A |
| 312 | A |
| 313 | A |
| 314 | A |
| 315 | A |
| 316 | A |
| 317 | A |
| 318 | A |
| 319 | A |
| 320 | A |
| 321 | A |
| 322 | D |
| 323 | A |
| 324 | A |
| 325 | B |
| 326 | A |
| 327 | A |
| 328 | A |
| 329 | B |
| 330 | A |
| 331 | C |
| 332 | A |
| 333 | A |
| 334 | A |
| 335 | A |
| 336 | A |
| 337 | D |
| 338 | A |
| 339 | A |
| 340 | A |
| 341 | A |
| 342 | A |
| 343 | A |
| 344 | A |
| 345 | A |
| 346 | A |
| 347 | B |
| 348 | A |
| 349 | A |
| 350 | A |
| 351 | B |
| 352 | A |
| 353 | B |
| 354 | A |
| 355 | D |
| 356 | A |
| 357 | A |
| 358 | B |
| 359 | A |
| 360 | D |
| 361 | A |
| 362 | B |
| 363 | A |
| 364 | A |
| 365 | A |
| 366 | B |
| 367 | B |
| 368 | B |
| 369 | A |
| 370 | A |
| 371 | A |
| 372 | A |
| 373 | B |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 385 | A |
| 386 | A |
| 387 | B |
| 388 | B |
| 389 | A |
| 390 | A |
| 391 | A |
| 392 | A |
| 393 | A |
| 394 | A |
| 395 | A |
| 396 | A |
| 397 | A |
| 398 | A |
| 399 | A |
| 400 | A |
| 401 | B |
| 402 | A |
| 403 | A |
| 404 | A |
| 405 | A |
| 406 | A |
| 407 | A |
| 408 | A |
| 409 | B |
| 410 | B |
| 411 | A |
| 412 | A |
| 413 | C |
| 414 | A |
| 415 | B |
| 416 | A |
| 417 | A |
| 418 | A |
| 419 | B |
| 420 | A |
| 421 | A |
| 422 | A |
| 423 | B |
| 424 | A |
| 425 | A |
| 426 | A |
| 427 | A |
| 428 | A |
| 429 | B |
| 430 | B |
| 431 | B |
| 432 | A |
| 433 | A |
| 434 | B |
| 435 | A |
| 436 | B |
| 437 | A |
| 438 | B |
| 439 | B |
| 440 | B |
| 441 | A |
| 442 | A |
| 443 | B |
| 444 | A |
| 445 | B |
| 446 | A |
| 447 | B |
| 448 | A |
| 449 | C |
| 450 | A |
| 451 | A |
| 452 | B |
| 453 | A |
| 454 | B |
| 455 | A |
| 456 | D |
| 457 | A |
| 458 | A |
| 459 | B |
| 461 | A |
| 462 | A |
| 463 | B |
| 464 | B |
| 465 | B |
| 466 | A |
| 467 | A |
| 468 | A |
| 469 | A |
| 470 | B |
| 471 | B |
| 472 | B |
| 473 | B |
| 474 | A |
| 475 | D |
| 476 | B |
| 477 | B |
| 478 | B |
| 479 | A |
| 480 | A |
| 481 | A |
| 482 | A |
| 483 | A |
| 484 | A |
| 485 | A |
| 486 | A |
| 487 | C |
| 488 | A |
| 489 | A |
| 490 | A |
| 491 | A |
| 492 | A |
| 493 | A |
| 494 | A |
| 495 | A |
| 496 | A |
| 497 | A |
| 498 | A |
| 499 | A |
| 500 | D |
| 501 | B |
| 502 | A |
| 503 | A |
| 504 | A |
| 505 | A |
| 506 | A |
| 507 | A |
| 508 | A |
| 509 | A |
| 510 | A |
| 511 | A |
| 512 | A |
| 513 | A |
| 514 | A |
| 515 | A |
| 516 | A |
| 517 | B |
| 518 | A |
| 519 | A |
| 520 | A |
| 521 | A |
| 522 | B |
| 523 | A |
| 524 | A |
| 525 | A |
| 526 | A |
| 527 | B |
| 528 | A |
| 529 | A |
| 530 | A |
| 531 | B |
| 532 | A |
| 533 | A |
| 534 | A |
| 535 | A |
| 536 | A |
| 537 | A |
| 538 | A |
| 539 | A |
| 540 | C |
| 541 | A |
| 542 | B |
| 543 | A |
| 544 | A |
| 545 | A |
| 546 | A |
| 547 | A |
| 548 | A |
| 549 | A |
| 550 | A |
| 551 | A |
| 552 | A |
| 553 | A |
| 554 | A |
| 555 | B |
| 556 | A |
| 557 | C |
| 558 | B |
| 559 | A |
| 560 | A |
| 561 | A |
| 562 | B |
| 563 | A |
| 564 | B |
| 565 | A |
| 566 | B |
| 567 | A |
| 568 | A |
| 569 | A |
| 570 | B |
| 571 | A |
| 572 | A |
| 573 | A |
| 574 | A |
| 575 | B |
| 576 | A |
| 577 | A |
| 578 | C |
| 579 | C |
| 580 | A |
| 581 | B |
| 582 | A |
| 583 | A |
| 584 | A |
| 585 | A |
| 586 | B |
| 587 | D |
| 588 | A |
| 589 | A |
| 590 | A |
| 591 | A |
| 592 | A |
| 593 | C |
| 594 | A |
| 595 | A |
| 596 | A |
| 597 | B |
| 598 | A |
| 599 | C |
| 600 | A |
| 601 | A |
| 602 | A |
| 603 | C |
| 604 | A |
| 605 | A |
| 606 | C |
| 607 | A |
| 608 | B |
| 609 | A |
| 610 | A |
| 611 | B |
| 612 | A |
| 613 | A |
| 614 | A |
| 615 | D |
| 616 | A |
| 617 | A |
| 618 | A |
| 619 | C |
| 620 | A |
| 621 | A |
| 622 | A |
| 623 | A |
| 624 | A |
| 625 | A |
| 626 | B |
| 627 | A |
| 628 | A |
| 629 | B |
| 630 | B |
| 631 | A |
| 632 | B |
| 633 | C |
| 634 | A |
| 635 | A |
| 636 | A |
| 637 | A |
| 638 | A |
| 639 | A |
| 640 | A |
| 641 | B |
| 642 | A |
| 643 | A |
| 644 | A |
| 645 | A |
| 646 | A |
| 647 | A |
| 648 | C |
| 649 | A |
| 650 | A |
| 651 | A |
| 652 | A |
| 653 | A |
| 654 | A |
| 655 | A |
| 656 | A |
| 657 | C |
| 658 | A |
| 659 | A |
| 660 | B |
| 661 | B |
| 662 | A |
| 663 | A |
| 664 | A |
| 665 | A |
| 666 | A |
| 667 | B |
| 668 | A |
| 669 | A |
| 670 | A |
| 671 | A |
| 672 | A |
| 673 | B |
| 674 | A |
| 676 | A |
| 677 | A |
| 678 | A |
| 679 | A |
| 680 | A |
| 681 | A |
| 682 | A |
| 683 | A |
| 684 | A |
| 685 | A |
| 686 | A |
| 687 | A |
| 688 | A |
| 689 | A |
| 690 | A |
| 691 | A |
| 692 | A |
| 693 | A |
| 694 | A |
| 695 | A |
| 696 | A |
| 697 | A |
| 698 | A |
| 699 | A |
| 700 | A |
| 701 | A |
| 702 | A |
| 703 | A |
| 704 | A |
| 705 | A |
| 706 | A |
| 708 | A |
| 709 | A |
| 710 | A |
| 711 | A |
| 712 | A |
| 713 | A |
| 714 | A |
| 715 | A |
| 716 | A |
| 717 | A |
| 718 | A |
| 719 | B |
| 720 | A |
| 721 | A |
| 722 | A |
| 723 | A |
| 724 | A |
| 725 | A |
| 726 | A |
| 727 | A |
| 728 | B |
| 729 | A |
| 730 | B |
| 731 | A |
| 732 | A |
| 733 | A |
| 734 | A |
| 735 | A |
| 736 | A |
| 737 | A |
| 738 | A |
| 739 | A |
| 740 | A |
| 741 | A |
| 742 | A |
| 743 | A |
| 744 | A |
| 745 | A |
| 746 | A |
| 747 | A |
| 748 | A |
| 749 | A |
| 750 | A |
| 751 | A |
| 752 | A |
| 753 | A |
| 754 | A |
| 755 | A |
| 756 | A |
| 757 | A |
| 758 | A |
| 759 | A |
| 760 | A |
| 761 | A |
| 762 | A |
| 763 | A |
| 764 | A |
| 765 | A |
| 766 | A |
| 767 | A |
| 768 | A |
| 769 | A |
| 770 | A |
| 771 | A |
| 772 | A |
| 773 | A |
| 774 | A |
| 775 | A |
| 776 | A |
| 777 | A |
| 778 | A |
| 779 | A |
| 780 | A |
| 781 | A |
| 782 | B |
| 783 | A |
| 784 | D |
| 785 | A |
| 786 | A |
| 787 | B |
| 788 | A |
| 789 | A |
| 790 | A |
| 791 | A |
| 792 | A |
| 793 | A |
| 794 | D |
| 795 | A |
| 796 | A |
| 797 | A |
| 798 | A |
| 799 | A |
| 800 | A |
| 801 | A |
| 802 | A |
| 803 | A |
| 804 | A |
| 805 | A |
| 806 | A |
| 807 | A |
| 808 | A |
| 809 | A |
| 810 | B |
| 811 | A |
| 812 | A |
| 813 | A |
| 814 | A |
| 815 | A |
| 816 | A |
| 817 | A |
| 818 | A |
| 819 | A |
| 820 | A |
| 821 | A |
| 822 | A |
| 823 | A |
| 824 | A |
| 825 | A |
| 826 | A |
| 827 | A |
| 828 | A |
| 829 | A |
| 830 | A |
| 831 | A |
| 832 | A |
| 833 | A |
| 834 | A |
| 835 | A |
| 836 | A |
| 837 | A |
| 838 | A |
| 839 | A |
| 840 | A |
| 841 | A |
| 842 | A |
| 843 | A |
| 844 | A |
| 845 | A |
| 846 | A |
| 847 | A |
| 848 | A |
| 849 | A |
| 850 | A |
| 851 | A |
| 852 | A |
| 853 | A |
| 854 | A |
| 855 | A |
| 856 | A |
| 857 | A |
| 858 | A |
| 859 | A |
| 860 | A |
| 861 | A |
| 862 | A |
| 863 | A |
| 864 | A |
| 865 | B |
| 866 | A |
| 867 | A |
| 868 | B |
| 869 | A |
| 870 | A |
| 871 | A |
| 872 | A |
| 873 | B |
| 874 | A |
| 875 | A |
| 876 | A |
| 877 | A |
| 878 | A |
| 879 | A |
| 880 | A |
| 881 | A |
| 882 | A |
| 883 | A |
| 884 | A |
| 885 | A |
| 886 | D |
| 887 | A |
| 888 | A |
| 889 | A |
| 890 | A |
| 891 | A |
| 892 | A |
| 893 | A |
| 894 | A |
| 895 | A |
| 896 | A |
| 897 | A |
| 898 | A |
| 899 | A |
| 900 | A |
| 901 | A |
| 902 | A |
| 903 | A |
| 904 | A |
| 905 | B |
| 906 | A |
| 907 | A |
| 908 | A |
| 909 | A |
| 910 | B |
| 911 | B |
| 912 | A |
| 913 | A |
| 914 | A |
| 915 | A |
| 916 | B |
| 917 | A |
| 918 | A |
| 919 | A |
| 920 | A |
| 921 | B |
| 922 | B |
| 923 | A |
| 924 | A |
| 925 | A |
| 926 | A |
| 927 | A |
| 928 | A |
| 929 | A |
| 930 | A |
| 931 | A |
| 932 | A |
| 933 | A |
| 934 | A |
| 935 | A |
| 936 | A |
| 937 | A |
| 938 | A |
| 939 | A |
| 940 | A |
| 941 | A |
| 942 | A |
| 943 | A |
| 944 | A |
| 945 | A |
| 946 | A |
| 947 | A |
| 948 | A |
| 949 | A |
| 950 | B |
| 951 | A |
| 952 | A |
| 953 | A |
| 954 | A |
| 955 | A |
| 956 | A |
| 957 | A |
| 958 | A |
| 959 | A |
| 960 | A |
| 961 | B |
| 962 | A |
| 963 | A |
| 964 | A |
| 965 | B |
| 966 | A |
| 967 | A |
| 968 | A |
| 969 | A |
| 970 | A |
| 971 | A |
| 972 | B |
| 973 | A |
| 974 | B |
| 975 | A |
| 976 | A |
| 977 | B |
| 978 | A |
| 979 | A |
| 980 | A |
| 981 | A |
| 982 | A |
| 983 | A |
| 984 | A |
| 985 | A |
| 986 | A |
| 987 | A |
| 988 | A |
| 989 | A |
| 990 | A |
| 991 | A |
| 992 | A |
| 993 | A |
| 994 | B |
| 995 | A |
| 996 | A |
| 997 | A |
| 998 | A |
| 999 | A |
| 1000 | A |
| 1001 | B |
| 1002 | A |
| 1003 | A |
| 1005 | C |
| 1006 | C |
| 1007 | A |
| 1008 | A |
| 1009 | A |
| 1011 | B |
| 1012 | A |
| 1013 | B |
| 1014 | A |
| 1015 | A |
| 1016 | A |
| 1017 | A |
| 1018 | A |
| 1019 | A |
| 1020 | A |
| 1021 | A |
| 1022 | A |
| 1023 | A |
| 1024 | A |
| 1025 | A |
| 1026 | A |
| 1027 | A |
| 1028 | A |
| 1029 | A |
| 1030 | A |
| 1031 | B |
| 1032 | C |
| 1033 | C |
| 1035 | A |
| 1036 | A |
| 1037 | A |
| 1038 | A |
| 1039 | C |
| 1040 | A |
| 1041 | A |
| 1042 | A |
| 1043 | A |
| 1044 | A |
| 1045 | A |
| 1046 | A |
| 1047 | A |
| 1048 | A |
| 1049 | A |
| 1050 | A |
| 1051 | A |
| 1052 | A |
| 1053 | A |
| 1054 | B |
| 1055 | B |
| 1056 | A |
| 1057 | A |
| 1058 | A |
| 1059 | A |
| 1060 | A |
| 1061 | A |
| 1062 | A |
| 1063 | A |
| 1064 | B |
| 1065 | A |
| 1066 | B |
| 1067 | C |
| 1068 | C |
| 1069 | C |
| 1070 | A |
| 1071 | A |
| 1072 | A |
| 1073 | A |
| 1074 | A |
| 1076 | A |
| 1077 | A |
| 1078 | A |
| 1079 | A |
| 1080 | A |
| 1081 | A |
| 1082 | A |
| 1083 | A |
| 1084 | A |
| 1085 | A |
| 1086 | A |
| 1087 | A |
| 1088 | B |
| 1089 | A |
| 1090 | A |
| 1091 | A |
| 1092 | A |
| 1093 | A |
| 1094 | A |
| 1095 | A |
| 1096 | A |
| 1097 | A |
| 1099 | A |
| 1100 | A |
| 1101 | A |
| 1102 | A |
| 1103 | A |
| 1104 | A |
| 1105 | A |
| 1106 | B |
| 1107 | A |
| 1108 | A |
| 1109 | B |
| 1111 | B |
| 1112 | A |
| 1113 | A |
| 1114 | A |
| 1115 | A |
| 1117 | A |
| 1118 | A |
| 1119 | A |
| 1120 | A |
| 1122 | A |
| 1123 | A |
| 1124 | A |
| 1125 | B |
| 1126 | A |
| 1128 | A |
| 1129 | A |
| 1130 | A |
| 1131 | C |
| 1132 | A |
| 1133 | A |
| 1134 | A |
| 1135 | A |
| 1136 | A |
| 1137 | B |
| 1138 | A |
| 1139 | A |
| 1140 | A |
| 1141 | A |
| 1142 | A |
| 1144 | A |
| 1145 | A |
| 1146 | A |
| 1147 | A |
| 1148 | A |
| 1149 | A |
| 1150 | B |
| 1151 | B |
| 1152 | A |
| 1153 | A |
| 1154 | A |
| 1155 | A |
| 1156 | A |
| 1157 | B |
| 1158 | A |
| 1159 | A |
| 1160 | A |
| 1161 | A |
| 1162 | A |
| 1163 | A |
| 1164 | A |
| 1165 | A |
| 1166 | B |
| 1167 | A |
| 1168 | B |
| 1169 | A |
| 1170 | A |
| 1171 | A |
| 1172 | A |
| 1173 | A |
| 1174 | A |
| 1175 | A |
| 1176 | A |
| 1177 | A |
| 1178 | A |
| 1179 | A |
| 1180 | A |
| 1181 | A |
| 1182 | A |
| 1183 | A |
| 1184 | B |
| 1185 | A |
| 1186 | A |
| 1187 | A |
| 1188 | A |
| 1189 | A |
| 1190 | B |
| 1191 | A |
| 1192 | A |
| 1193 | A |
| 1194 | A |
| 1195 | A |
| 1196 | A |
| 1197 | A |
| 1198 | A |
| 1199 | A |
| 1200 | A |
| 1201 | A |
| 1202 | A |
| 1203 | A |
| 1204 | C |
| 1205 | A |
| 1206 | A |
| 1207 | A |
| 1208 | A |
| 1209 | A |
| 1210 | A |
| 1211 | A |
| 1212 | A |
| 1213 | A |
| 1214 | A |
| 1215 | B |
| 1216 | A |
| 1217 | A |
| 1218 | A |
| 1219 | B |
| 1220 | B |
| 1221 | B |
| 1222 | A |
| 1223 | A |
| 1224 | A |
| 1225 | C |
| 1226 | B |
| 1227 | B |
| 1228 | C |
| 1229 | C |
| 1230 | B |
| 1231 | A |
| 1232 | A |
| 1233 | B |
| 1234 | A |
| 1235 | C |
| 1236 | C |
| 1237 | A |
| 1238 | C |
| 1239 | C |
| 1240 | A |
| 1241 | C |
| 1242 | A |
| 1243 | A |
| 1244 | A |
| 1245 | A |
| 1246 | B |
| 1248 | B |
| 1249 | A |
| 1250 | A |
| 1251 | A |
| 1252 | A |
| 1253 | A |
| 1254 | A |
| 1255 | A |
| 1256 | C |
| 1257 | B |
| 1258 | B |
| 1259 | A |
| 1260 | A |
| 1261 | A |
| 1262 | A |
| 1263 | A |
| 1264 | C |
| 1265 | A |
| 1266 | C |
| 1267 | A |
| 1269 | A |
| 1270 | B |
| 1271 | C |
| 1272 | C |
| 1273 | C |
| 1274 | A |
| 1275 | A |
| 1276 | B |
| 1277 | C |
| 1278 | A |
| 1279 | A |

### Biological Example 5: PI3Kα Kinase Inhibitors Vs. Breaker Compounds

The present disclosure provides various compounds capable of breaking, disrupting, inhibiting, and/or preventing an interaction between a PI3K protein (e.g., PI3Kα) and a small GTPase (e.g., KRAS). Such compounds may be referred to as “PI3Kα breakers.”

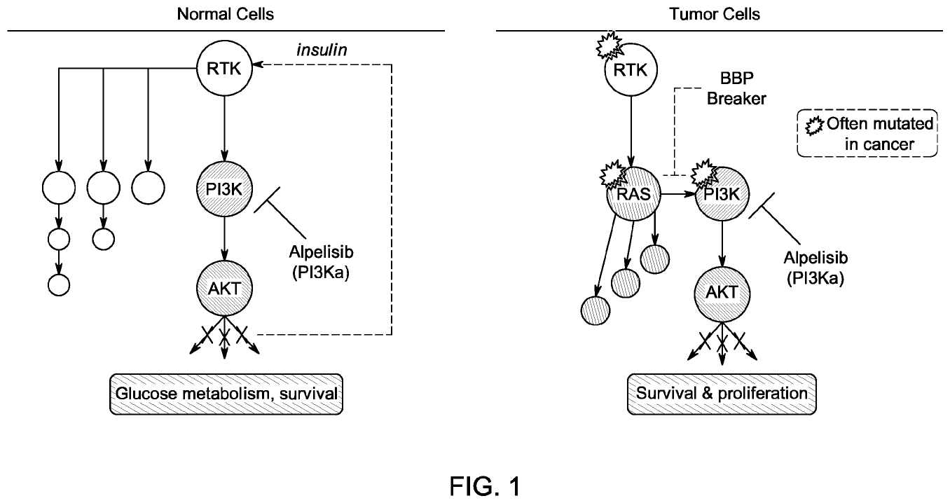

As shown in the left panel of FIG. 1, in normal cells, activation of receptor tyrosine kinases (RTKs) leads to PI3Kα-mediated phosphorylation and activation of AKT (also known as protein kinase B, PKB). The PI3K/AKT pathway plays an important role in glucose metabolism: when glucose levels are elevated, insulin is released from the pancreas; subsequent activation of insulin receptors activates the PI3K/AKT pathway. In normal cells, inhibition of PI3Kα with a PI3Kα inhibitor such as alpelisib can reduce glucose uptake, in turn leading to increased secretion of insulin and subsequent activation of insulin receptors. In tumor cells, the impact of inhibition of PI3Kα can be provision of a survival mechanism for tumor cells and thus limitation of the therapeutic efficacy of the PI3Kα inhibitor. In contrast, “breaking” the interaction between RAS and PI3Kα should have the effect of avoiding hyperglycemia and insulin-driven resistance by selectively targeting tumor cells (see right panel of FIG. 1). Accordingly, PI3Kα breakers, such as compounds provided herein, should selectively bind to PI3Kα, such as to the Ras binding domain (RBD) of PI3Kα. Such compounds should have no binding affinity to RAS proteins such as KRAS, and should not affect the kinase activity of PI3Kα. Surface plasmon resonance (SPR) and isothermal titration calorimetry (ITC) confirm that RAS binds to PI3Kα with about 10 μM affinity, and that PI3Kα breakers binding to PI3Kα block its interaction with RAS.

Table 8 compares the pAKT IC50 and cell viability for Compound 58, a PI3Kα breaker, and the PI3Kα inhibitor alpelisib in the breast cancer estrogen receptor positive (ER+) BT474 cell line, which harbors a HER2 amplification and PIK3CA linker domain K111N mutation. As shown in Table 8, similar inhibition of pAKT (IC₅₀) can be achieved with lower concentrations of a PI3Kα breaker than alpelisib, suggesting that disrupting the interaction of RAS with PI3Kα can be at least as potent as the PI3Kα kinase domain inhibitor. Importantly, the potency on pAKT translates well into a loss in cellular viability.

TABLE 8
Comparison between PI3Kα breaker and
PI3Kα inhibitor Alpelisib.

| | Compound 58 | Alpelisib |
|---|---|---|
| pAKT (IC50, nM) | 34 | 169 |
| Cell Viability (nM) | 67 | 744 |

### Biological Example 6: In Vitro Activity of PI3Kα Breakers

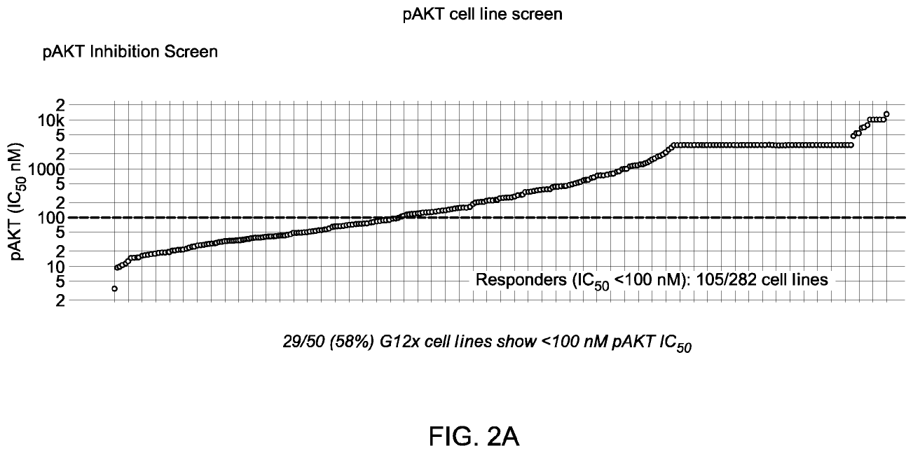

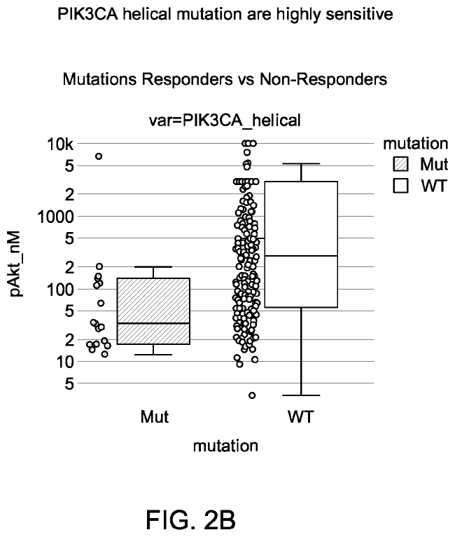

The pAKT inhibition of Compound 44 or Compound 58 in a diverse panel of 282 human tumor cell lines from different cancer indications with varied genetic backgrounds was evaluated to determine factors that may influence sensitivity and resistance (FIG. 2A). PI3K pathway signaling in a 2D culture system was assessed by measuring pAKT (Ser473) protein levels following 4 hours of treatment with nine concentrations of Compound 44 or Compound 58 by homogeneous time resolved fluorescence (HTRF). The pAKT IC₅₀ of each cell line was calculated using GraphPad prism software. 105 cell lines had pAKT IC₅₀<100 nM and were considered sensitive to Compound 44 or Compound 58. Notably, 105 of 282 (37%) of screened cell lines are responders. Further, 29 or 50 (58%) of screen KRAS G12X cell lines are responders. Bioinformatic analysis revealed that the presence of mutations in the helical domain of PIK3CA was a strong predictor of sensitivity to Compound 44 or Compound 58 in this panel (FIG. 2B).

Table 9 summarizes the pAKT IC50 for Compound 58, a PI3Kα breaker compound, in a panel of selected human cancer cell lines that have KRAS, HER2, HER3, or EGFR alterations, with or without PIK3CA activating helical (E542K), kinase (H1047L), C2 (C420R), or linker (G118D) domain mutations. As shown in Table 9, cell lines across a variety of indications, including esophageal, gastric, colorectal, breast, oral, head and neck, skin, and non-small cell lung cancers, display sensitivity to Compound 58 with a pAKT IC₅₀<100 nM.

TABLE 9
In vitro cancer cell line PI3Ka breaker pAKT IC₅₀ results.

| | Cancer | KRAS, HER2, HER3, or | PIK3CA | Compound 58 |
|---|---|---|---|---|
| Cell Line | Indication | EGFR Alterations | Mutations | pAKT IC₅₀ (nM) |
| KYSE-410 | Esophageal | KRAS G12C mutation and | None | 35 |
| | | HER2 amplification | | |
| SNU-601 | Gastric | KRAS G12D mutation | E542K | 27 |
| GP2d | Colorectal | KRAS G12D mutation | H1047L | 42 |
| SNU-16 | Gastric | KRAS G12D mutation | None | 73 |
| JIMT-1 | Breast | HER2 amplification | C420R | 51 |
| N87 | Gastric | HER2 amplification | None | 25 |
| CAL27 | Oral squamous cell | HER3 positive | None | 81 |
| FaDu | Head and neck | HER3 D297H mutation | None | 78 |
| A431 | Skin squamous cell | EGFR amplification | None | 39 |
| H1975- | Non-small cell | EGFR L858R, T790M, and | G118D | 26 |
| C797S | lung | C797S mutations | | |

### Biological Example 7: PI3Kα Breakers are Orally Bioavailable and Achieve Near Complete Inhibition of Signaling in Tumors at 100 mg/kg without Risk of Hyperglycemia

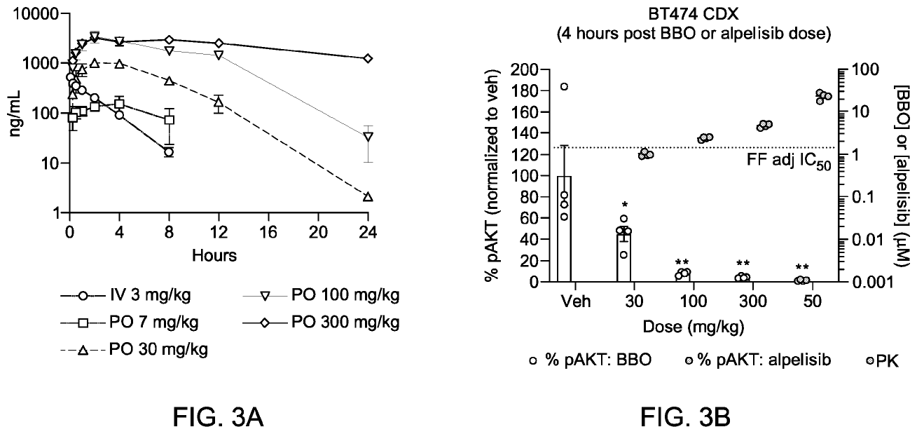

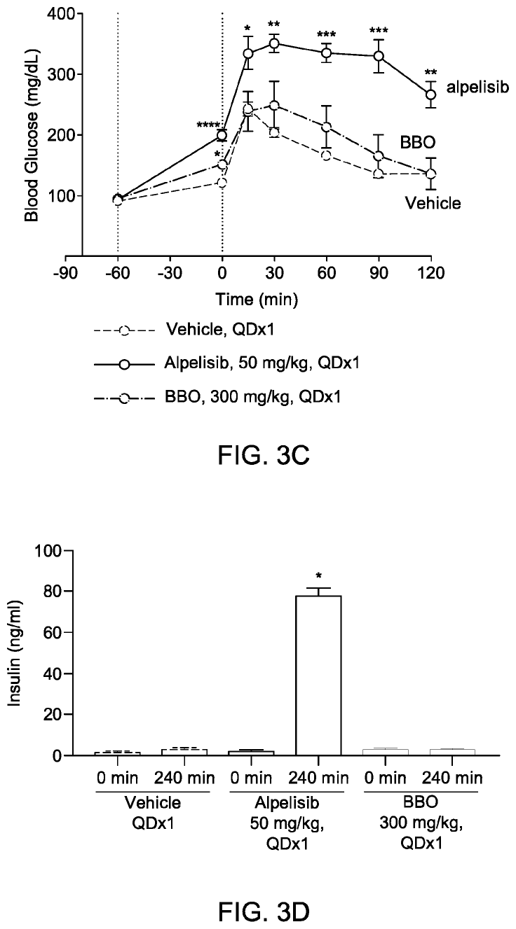

The pharmacokinetic profile of Compound 58 was evaluated in mice (FIG. 3A). Male CD-1 mice (3 mice per group) were administered a single dose of Compound 58 at 3 mg/kg by intravenous (IV) injection or 7, 30, 100, or 300 mg/kg by oral gavage. Blood was collected at 0.0833 (IV only), 0.25, 0.5, 1, 2, 4, 8, 12, and/or 24 hours after each dose and processed to plasma. Compound 58 concentrations were then determined with LC-MS/MS. The Cₘₐₓ following oral doses of 7, 30, 100, and 300 mg/kg was 166, 1084, 3457, and 3257 ng/mL, respectively. The AUC_{inf} following oral doses of 7, 30, 100, and 300 mg/kg was 1418, 8453, 34,866, and 78,735 hr*ng/mL, respectively. These results demonstrated that the Cₘₐₓ increased dose proportionally up to 100 mg/kg, the AUC_{inf} of Compound 58 increased dose proportionally up to 300 mg/kg, and that there was potential saturation of both absorption and clearance at 300 mg/kg. The F % following oral doses of 7, 30, 100, and 300 mg/kg was 51%, 71%, 88%, and 66%, respectively, indicating that Compound 58 had high oral bioavailability at all tested dose levels.

An in vivo study was conducted to evaluate the pharmacodynamic activity of Compound 58 in the breast cancer estrogen receptor positive (ER+) BT474 cell line-derived xenograft (CDX) model, which harbors a HER2 amplification and PIK3CA linker domain K111N mutation (FIG. 3B). NOD/SCID mice were implanted subcutaneously with BT474 human tumor cells and were injected subcutaneously with estradiol benzoate twice a week to support tumor growth. When tumors reached a mean size of 286 mm³, mice were randomized into treatment groups (4 mice per group) and administered one oral dose of vehicle, 30 mg/kg Compound 58, 100 mg/kg Compound 58, 300 mg/kg Compound 58, or 50 mg/kg alpelisib. Alpelisib is a PI3Kα kinase domain inhibitor and was included as a positive control. Whole blood and tumors were collected 4 hours post the dose. Blood was processed to plasma and compound concentrations were determined with LC-MS/MS. Tumors were processed into lysates and pAKT levels were measured using the Meso Scale Diagnostics (MSD) multi-spot pAKT (Ser473)/total AKT assay whole cell lysate kit.

Single oral administration of Compound 58 resulted in a statistically significant reduction in pAKT compared to the vehicle group at all dose levels. pAKT was inhibited by 40%, 87%, and 93% at 4 hours following the oral administration of 30, 100, and 300 mg/kg Compound 58, respectively, and by 97% at 4 hours following the oral administration of 50 mg/kg alpelisib. The Compound 58 in vivo EC₅₀ of 1163 nM was very similar to the in vitro Compound 58 BT474 pAKT free fraction adjusted IC₅₀ of 1438 nM. These results demonstrated that Compound 58 achieved near complete inhibition of pAKT at 100 mg/kg in this model.

Two in vivo studies were performed to determine whether Compound 58 administration induces hyperglycemia and hyperinsulinemia in mice like the PI3Kα kinase domain inhibitor alpelisib (FIGS. 3C and 3D). In the first study, an oral glucose tolerance test was performed in fasted mice following the administration of compounds to determine if Compound 58 induces hyperglycemia (FIG. 3C). Male C57/BL6J mice were fasted overnight for 16 hours and approximately 5 μL of whole blood was collected via tail snip to measure fasted blood glucose levels using a glucometer. Mice were randomized into three groups (8 mice per group) based on blood glucose levels and then administered one oral dose of vehicle, 50 mg/kg alpelisib, or 300 mg/kg Compound 58. One hour later, blood was collected for a glucose measurement and then a single oral dose of a 2 g/kg glucose solution was administered. Blood glucose measurements were performed at 15, 30, 60, 90, and 120 minutes after the administration of the glucose solution. Blood glucose levels were significantly elevated at all timepoints post the glucose dose in mice treated with 50 mg/kg alpelisib but not 300 mg/kg Compound 58. This indicated that inhibition of the RAS:PI3Kα interaction by Compound 58 treatment did not induce hyperglycemia.

In the second study, serum insulin levels were measured in fed mice following administration of compounds to determine if Compound 58 induces hyperinsulinemia like the PI3Kα kinase domain inhibitor alpelisib (FIG. 3D). Approximately 5 μL of whole blood was collected via tail snip from male C57/BL6J mice and used to measure fed blood glucose levels using a glucometer. Mice were randomized into three groups (8 mice per group) based on blood glucose levels and then administered one oral dose of vehicle, 50 mg/kg alpelisib, or 300 mg/kg Compound 58. 240 minutes later, whole blood was collected and processed to serum and insulin levels were measured using an ELISA. Serum insulin levels were significantly elevated 240 minutes following administration of 50 mg/kg alpelisib but not 300 mg/kg Compound 58. This indicated that inhibition of the RAS:PI3Kα interaction by Compound 58 treatment did not induce hyperinsulinemia.

These two in vivo pharmacology studies demonstrated that unlike alpelisib, PI3Kα breaker compounds do not affect glucose metabolism.

### Biological Example 8: PI3Kα Breakers are Efficacious in Xenograft Models with KRAS Mutations

Several in vivo studies were conducted to assess the anti-tumor activity of Compound 58 as a monotherapy in KRAS mutated CDX models with or without additional alterations such as HER2 amplification or PIK3CA mutations. Immunocompromised female mice were implanted subcutaneously with KYSE-410, GP2d, SNU-601, or SNU-16 human tumor cells. When tumors reached a mean size of 175 to 200 mm³, mice were randomized into treatment groups (10 mice per group) and dosed orally once per day with the indicated levels of Compound 58. Mouse tumor volumes and body weights were measured twice weekly until day 28. Tumor growth inhibition (TGI), an indicator of anti-tumor effectiveness, was calculated for each treatment (T) versus control (C) group using the first day of dosing (0) and indicated measurement day (i) mean tumor volume measurements with the formula: TGI (%)=(1−((Tᵢ-T₀)/(Cᵢ-C₀)))×100. TGI was only reported if ≤100%. Tumor regression was also assessed and defined as a tumor with a smaller tumor volume on the indicated day of the study compared to the first day of dosing. For statistical analyses comparing the vehicle group to all other groups, two-way repeated measures analysis of variance (ANOVA) followed by post hoc Dunnett's multiple comparisons test of the means was applied. Treatments were not considered tolerated if >20% of the mice in the group had ≥20% body weight loss or >20% of the mice in the group spontaneously died or had any clinical signs of distress that required euthanasia.

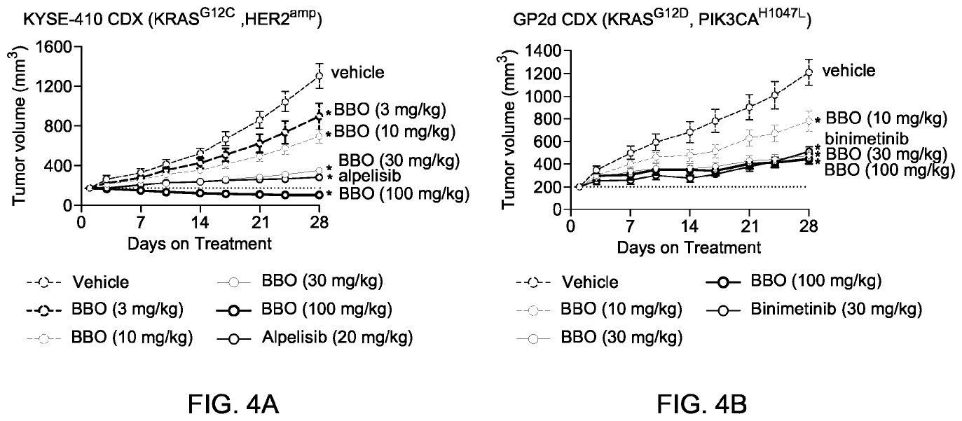

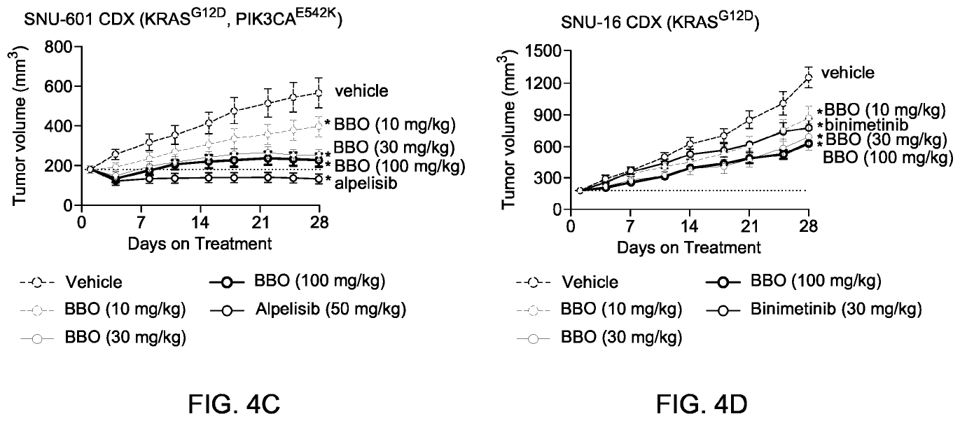

In NOD/SCID mice bearing esophageal carcinoma KYSE-410 subcutaneous xenograft tumors, which harbor a KRAS G12C mutation and HER2 amplification, a dose dependent reduction in tumor volume was observed (FIG. 4A). Following once daily oral administration of Compound 58, a statistically significant reduction in tumor volume compared to the vehicle group was observed in the 3, 10, 30, and 100 mg/kg Compound 58 treatment groups compared to the vehicle group, with 36% TGI, 54% TGI, 85% TGI, and 42% tumor regression, respectively, on day 28. There was no impact of treatment on body weight and all treatments were well tolerated.

In BALB/c nude mice bearing colorectal carcinoma GP2d subcutaneous xenograft tumors, which harbor KRAS G12D and PIK3CA kinase domain H1047L mutations, a dose dependent reduction in tumor volume was observed (FIG. 4B). Following once daily oral administration of Compound 58, a statistically significant reduction in tumor volume compared to the vehicle group was observed in the 10, 30, and 100 mg/kg Compound 58 treatment groups compared to the vehicle group, with 43% TGI, 73% TGI, and 76% TGI, respectively, on day 28. There was no impact of treatment on body weight and all treatments were well tolerated.

In BALB/c nude mice bearing colorectal carcinoma SNU-601 subcutaneous xenograft tumors, which harbor KRAS G12D and PIK3CA helical domain E542K mutations, a dose dependent reduction in tumor volume was observed (FIG. 4C). Following once daily oral administration of Compound 58, a statistically significant reduction in tumor volume compared to the vehicle group was observed in the 10, 30, and 100 mg/kg Compound 58 treatment groups compared to the vehicle group, with 44% TGI, 81% TGI, and 88% TGI, respectively, on day 28. There was no impact of treatment on body weight and all treatments were well tolerated.

In BALB/c nude mice bearing colorectal carcinoma SNU-16 subcutaneous xenograft tumors, which harbor a KRAS G12D mutation, a dose dependent reduction in tumor volume was observed (FIG. 4D). Following once daily oral administration of Compound 58, a statistically significant reduction in tumor volume compared to the vehicle group was observed in the 10, 30, and 100 mg/kg Compound 58 treatment groups compared to the vehicle group, with 36% TGI, 52% TGI, and 58% TGI, respectively, on day 28. There was no impact of treatment on body weight and all treatments were well tolerated.

These in vivo efficacy studies demonstrated that Compound 58 administered as a monotherapy had statistically significant anti-tumor efficacy in multiple KRAS mutant subcutaneous xenograft models with or without additional alterations such as HER2 amplification or PIK3CA mutations at well-tolerated doses.

### Biological Example 9: PI3Kα Breakers are Efficacious in Xenograft Models with HER2 Alterations

Several in vivo studies were conducted to assess the anti-tumor activity of Compound 58 as a monotherapy in HER2+ CDX and patient-derived xenograft (PDX) models. Immunocompromised NOD/SCID or BALB/c nude female mice were implanted subcutaneously with JIMT-1 or N87 human tumor cells, respectively. When tumors reached a mean size of 160 to 190 mm³, mice were randomized into treatment groups (10 mice per group) and dosed orally once per day with vehicle or 100 mg/kg Compound 58. Mouse tumor volumes were measured twice weekly until day 28 (JIMT-1) or day 21 (N87). Immunocompromised athymic nude mice were implanted subcutaneously with ˜70 mg ST2167 or ST340 PDX tumor fragments harvested from stock mice. When tumors reached a mean size of 200-260 mm³, mice were randomized into treatment groups (3 mice per group) and dosed orally once per day with vehicle or 100 mg/kg Compound 58. Mouse tumor volumes were measured twice weekly until day 14 (ST2167) or day 17 (ST340). TGI, an indicator of anti-tumor effectiveness, was calculated for each treatment (T) versus control (C) group using the first day of dosing (0) and indicated measurement day (i) mean tumor volume measurements as described in Biological Example 8.

Table 10 summarizes the monotherapy anti-tumor activity of Compound 58, a PI3Kα breaker, in several HER2+ CDX and PDX models, with or without PIK3CA activating C2 (C420R), kinase (H1047R), or helical (E545Q) domain mutations. The JIMT-1 and N87 CDX models harbor HER2 amplifications, and the ST2167 and ST340 PDX tumors were HER2+ by immunohistochemistry (IHC). As shown in Table 10, all four breast and gastric models were sensitive to Compound 58 with TGIs≥70%. Activity was observed in both estrogen receptor negative (ER−) and estrogen receptor positive (ER+) breast xenograft models.

TABLE 10
In vivo PI3Ka breaker monotherapy anti-tumor activity
in xenograft models with HER2 alterations.

| | | | | TGI (100 mg/kg |
|---|---|---|---|---|
| | Cancer | | PIK3CA | Compound 58, |
| Model | Indication | HER2 Status | Mutations | QD, po) |
| JIMT-1 | Breast (ER−) | Amplified | C420R | 84% |
| CDX | | | | |
| N87 | Gastric | Amplified | None | 87% |
| CDX | | | | |
| ST2167 | Breast (ER−) | Positive by IHC | H1047R | 70% |
| PDX | | | | |
| ST340 | Breast (ER+) | Positive by IHC | E545Q | 86% |
| PDX | | | | |

These in vivo efficacy studies demonstrated that Compound 58 administered as a monotherapy had anti-tumor efficacy in breast and gastric HER2+ CDX or PDX models with or without PIK3CA mutations.

### Biological Example 10: PI3Kα Breakers are Efficacious in Xenograft Models with HER3 Alterations

Two in vivo studies were conducted to assess the anti-tumor activity of Compound 58 as a monotherapy in CDX models with alterations in HER3. Immunocompromised BALB/c nude female mice were implanted subcutaneously with CAL27 or FaDu human tumor cells. When tumors reached a mean size of 170 to 200 mm³, mice were randomized into treatment groups (10 mice per group) and dosed orally once per day with vehicle or 100 mg/kg Compound 58. Mouse tumor volumes were measured twice weekly until day 28. TGI, an indicator of anti-tumor effectiveness, was calculated for each treatment (T) versus control (C) group using the first day of dosing (0) and indicated measurement day (i) mean tumor volume measurements as described in Biological Example 8.

Table 11 summarizes the monotherapy anti-tumor activity of Compound 58, a PI3Kα breaker, in two CDX models with HER3 alterations. The CAL27 cell line was HER3+ by flow cytometry, and the FaDu CDX model harbors a HER3 D297H mutation. As shown in Table 11, both models were sensitive to Compound 58 with TGIs≥90%.

TABLE 11
In vivo PI3Ka breaker monotherapy anti-tumor activity
in xenograft models with HER3 alterations.

| | | | | TGI (100 mg/kg |
|---|---|---|---|---|
| | | | PIK3CA | Compound 58, |
| Model | Cancer Indication | HER3 Status | Mutations | QD, po) |
| CAL27 CDX | Oral squamous cell | Positive by flow cytometry | None | 90% |
| FaDu CDX | Head and neck | D297H mutation | None | 93% |

These in vivo efficacy studies demonstrated that Compound 58 administered as a monotherapy had anti-tumor efficacy in oral squamous cell carcinoma and head and neck cancer CDX models with HER3 alterations. Biological Example 11: PI3Kα breakers are efficacious in xenograft models with EGFR alterations

Two in vivo studies were conducted to assess the anti-tumor activity of Compound 58 as a monotherapy in CDX models with alterations in EGFR. Immunocompromised BALB/c nude or NOD/SCID female mice were implanted subcutaneously with A431 or H1975-C797S human tumor cells, respectively. When tumors reached a mean size of 180 to 200 mm³, mice were randomized into treatment groups (10 mice per group) and dosed orally once per day with vehicle or 100 mg/kg Compound 58. Mouse tumor volumes were measured twice weekly until day 28. TGI, an indicator of anti-tumor effectiveness, was calculated for each treatment (T) versus control (C) group using the first day of dosing (0) and indicated measurement day (i) mean tumor volume measurements as described in Biological Example 8.

Table 12 summarizes the monotherapy anti-tumor activity of Compound 58, a PI3Kα breaker, in two CDX models with EGFR alterations. The A431 CDX model harbors an EGFR amplification. For the H1975-C797S CDX model, the EGFR C797S mutation was engineered using CRISPR into the parental H1975 cell line, which harbors EGFR L858R and T790M mutations and a PIK3CA linker domain G118D mutation. The EGFR C797S mutation reduces binding of the third generation EGFR inhibitor osimertinib, so the H1975-C797S CDX model is resistant to osimertinib. As shown in Table 12, both models were sensitive to Compound 58 with TGIs≥64%.

TABLE 12
In vivo PI3Ka breaker monotherapy anti-tumor activity
in xenograft models with EGFR alterations.

| | | | | TGI (100 mg/kg |
|---|---|---|---|---|
| | Cancer | | PIK3CA | Compound 58, |
| Model | Indication | EGFR Status | Mutations | QD, po) |
| A431 CDX | Skin squamous cell | Amplified | None | 66% |
| H1975-C797S CDX | Non-small cell lung | L858R, T790M, and | G118D | 64% |
| | | C797S mutations | | |

These in vivo efficacy studies demonstrated that Compound 58 administered as a monotherapy had anti-tumor efficacy in skin squamous cell carcinoma and non-small cell lung cancer CDX models with EGFR alterations.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

LENGTHY TABLES
The patent application contains a lengthy table section. A copy of the table is available in electronic form from the USPTO web site (https://seqdata.uspto.gov/?pageRequest=docDetail&DocID=US20250230138A1). An electronic copy of the table will also be available from the USPTO upon request and payment of the fee set forth in 37 CFR 1.19(b)(3).

1. A compound of formula I: 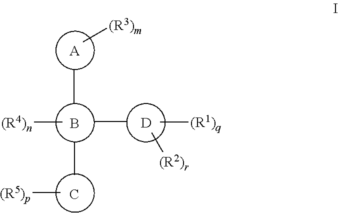
or a salt (e.g., pharmaceutically acceptable salt) thereof, wherein:
Ring A is selected from phenyl and a 5- to 6-membered heteroaryl ring having 1-3 heteroatoms independently selected from nitrogen, oxygen, and sulfur;
Ring B is selected from a 9- to 10-membered bicyclic ring that comprises at least one 5- or 6-membered heteroaryl ring, a 6-membered heteroaryl ring having 1-2 nitrogen atoms, and phenyl;
Ring C is selected from phenyl; a 5- to 6-membered heteroaryl ring having 1-3 heteroatoms independently selected from nitrogen, oxygen, and sulfur; a 5- to 8-membered bicyclic carbocyclic ring; a 4- to 8-membered heterocyclic ring having 1-2 heteroatoms independently selected from nitrogen, oxygen, and sulfur; and a 7- to 10-membered spirofused heterocyclic ring having 1-3 heteroatoms independently selected from nitrogen, oxygen, and sulfur, wherein each of the phenyl, heteroaryl, and heterocyclic rings is optionally fused to Ring E;
Ring D is selected from a 5- to 6-membered heteroaryl ring having 1-3 heteroatoms independently selected from nitrogen, oxygen, and sulfur and phenyl, wherein each of the heteroaryl and phenyl rings is optionally fused to Ring F;
Ring E is selected from a 5- to 6-membered carbocyclic ring; a 5- to 7-membered heterocyclic ring having 1-2 heteroatoms independently selected from nitrogen, oxygen, and sulfur; 7- to 10-membered spirofused heterocyclic ring having 1-3 heteroatoms independently selected from nitrogen, oxygen, and sulfur; and a 5- to 6-membered heteroaryl ring having 1-3 heteroatoms independently selected from nitrogen, oxygen, and sulfur, wherein Ring E is substituted by s instances of R^{5′};
Ring F is selected from phenyl; a 5- to 6-membered carbocyclic ring; a 5- to 7-membered heterocyclic ring having 1-2 heteroatoms independently selected from nitrogen, oxygen, and sulfur; and a 5- to 6-membered heteroaryl ring having 1-3 heteroatoms independently selected from nitrogen, oxygen, and sulfur, wherein Ring F is substituted by u instances of -L-W and y instances of R^{2′};
R¹ is selected from -L-W, Ring D′, or a bivalent C₁₋₆ aliphatic chain substituted with Ring D′;
each -L-W is —CN, or:
each L is independently a bivalent straight or branched C₁₋₈ aliphatic chain wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—;
each W is independently hydrogen, halogen, —CN, or an optionally substituted 3-10 membered monocyclic or bicyclic, saturated, partially unsaturated, or aryl ring having 0-3 heteroatoms independently selected from nitrogen, oxygen, or sulfur;
each X is independently halogen, —OR, or —CN;
each Ring D′ is independently a 4- to 6-membered carbocyclic ring or a 4- to 6-membered heterocyclic ring having 1-2 heteroatoms independently selected from nitrogen, oxygen, and sulfur, wherein Ring D′ is substituted with t instances of -L-W;
each R² and R^{2′} is independently selected from oxo, halogen, —CN, —OR, and C₁₋₆ alkyl;
each R³ is independently selected from oxo, halogen, —CN, —OR, —O(CH₂)ᵥCy, —OCH₂CH₂OR, and optionally substituted C₁₋₆ aliphatic;
each Cy is independently a 5- to 6-membered heteroaryl ring having 1-3 heteroatoms independently selected from nitrogen, oxygen, and sulfur; a 3- to 6-membered carbocyclic ring; or a 4- to 6-membered heterocyclic ring having 1-2 heteroatoms independently selected from nitrogen, oxygen, and sulfur, wherein Cy is substituted with 0-2 instances of R⁶;
each R⁴ is independently selected from halogen and optionally substituted C₁₋₆ aliphatic;
each of R⁵ and R^{5′} is independently selected from oxo, =NH, —CN, halogen, —OR, —N(R)₂, —SR, —C(O)R, —N(R)C(O)R, —(CH₂)ₓC(O)N(R)₂, —C(O)N(R)₂, —C(O)N(R)(CH₂)ₓCy, —(CH₂)ₓC(O)Cy, —OC(O)R, —C(O)OR, —SO₂R, —N(R)SO₂R, —N═S(O)(R)₂, —SO₂N(R)₂, —P(O)R₂, —(CH₂)ₓCy, —O(CH₂)ₓCy, and optionally substituted C₁₋₆ aliphatic;
each R⁶ is independently selected from oxo, —CN, halogen, —OR, —N(R)₂, —SR, —C(O)R, —N(R)C(O)R, —C(O)N(R)₂, —OC(O)R, —C(O)OR, —SO₂R, —N(R)SO₂R, —SO₂N(R)₂, and an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
each R is independently hydrogen or an optionally substituted group selected from C₁₋₆ aliphatic; a 3- to 6-membered carbocyclic ring; phenyl; a 3- to 6-membered heterocyclic ring having 1-3 heteroatoms independently selected from nitrogen, oxygen, and sulfur; a 5- to 6-membered heteroaryl ring having 1-3 heteroatoms independently selected from nitrogen, oxygen, and sulfur; a 10-membered aryl ring; and a 9- to 10-membered heteroaryl ring having 1-3 heteroatoms independently selected from nitrogen, oxygen, and sulfur;
m is 0, 1, 2, or 3;
n is 0, 1, or 2;
p is 0, 1, 2, or 3;
q is 0 or 1;
r is 0, 1, or 2;
s is 0, 1, 2, or 3;
t is 0, 1, or 2;
u is 0 or 1;
each v is independently 0, 1, or 2;
each x is independently 0, 1, or 2; and
y is 0, 1, or 2.

2. The compound of claim 1, wherein the compound is a compound according to Formula IA: 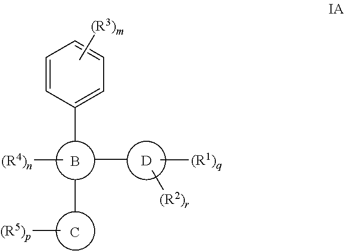
or a salt (e.g., pharmaceutically acceptable salt) thereof.

3. The compound of claim 2, wherein the compound is a compound according to Formula IA1: 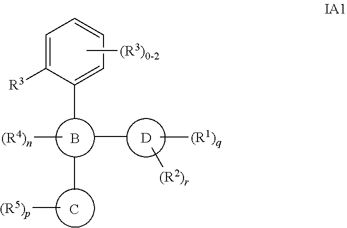
or a salt (e.g., pharmaceutically acceptable salt) thereof.

4. The compound of claim 1, wherein the compound is a compound according to Formula IB: 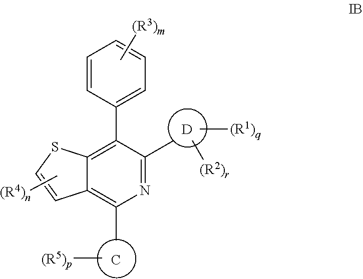
or a salt (e.g., pharmaceutically acceptable salt) thereof.

5. The compound of claim 4, wherein the compound is a compound according to Formula IB1: 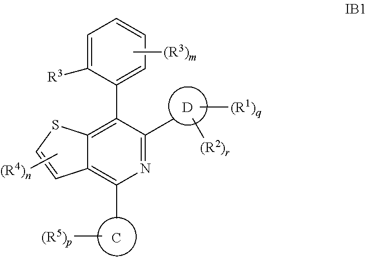
or a salt (e.g., pharmaceutically acceptable salt) thereof.

6. The compound of claim 1, wherein the compound is a compound according to Formula IC: 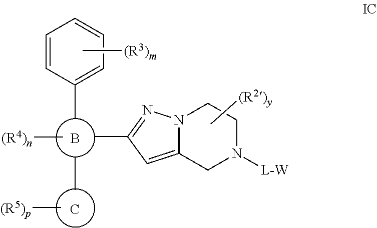
or a salt (e.g., pharmaceutically acceptable salt) thereof.

7. The compound of claim 6, wherein the compound is a compound according to Formula IC1: 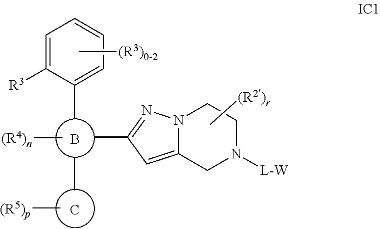
or a salt (e.g., pharmaceutically acceptable salt) thereof.

8. The compound of claim 1, wherein the compound is a compound according to Formula ID: 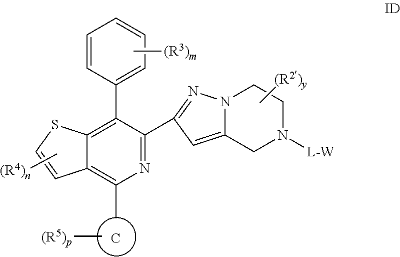
or a salt (e.g., pharmaceutically acceptable salt) thereof.

9. The compound of claim 8, wherein the compound is a compound according to Formula ID1: 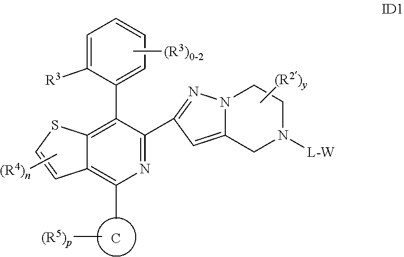
or a salt (e.g., pharmaceutically acceptable salt) thereof.

10. The compound of claim 1, wherein the compound is a compound according to Formula IE: 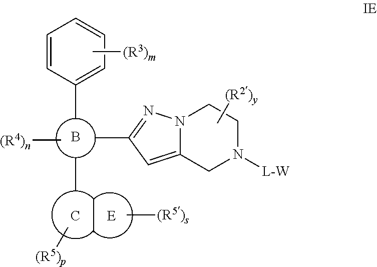
or a salt (e.g., pharmaceutically acceptable salt) thereof.

11. The compound of claim 10, wherein the compound is a compound according to Formula IE1: 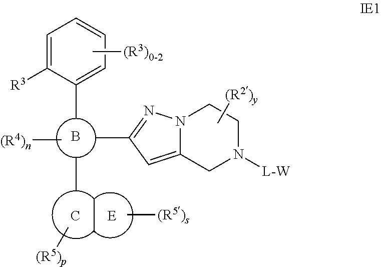
or a salt (e.g., pharmaceutically acceptable salt) thereof.

12. The compound of claim 1, wherein the compound is a compound according to Formula IF: 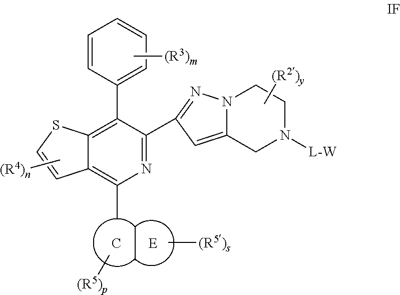
or a salt (e.g., pharmaceutically acceptable salt) thereof.

13. The compound of claim 12, wherein the compound is a compound according to Formula IF1: 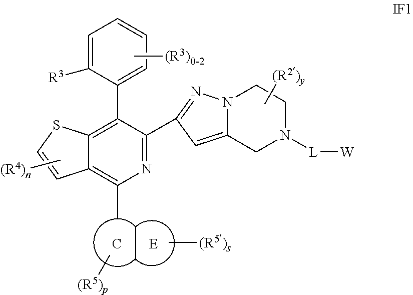
or a salt (e.g., pharmaceutically acceptable salt) thereof.

14. The compound of claim 1, wherein the compound is a compound according to one of Formulae I-a, I-b, I-c, I-d I-e I-f I-g, I-h, I-i, I-j, or I-k: 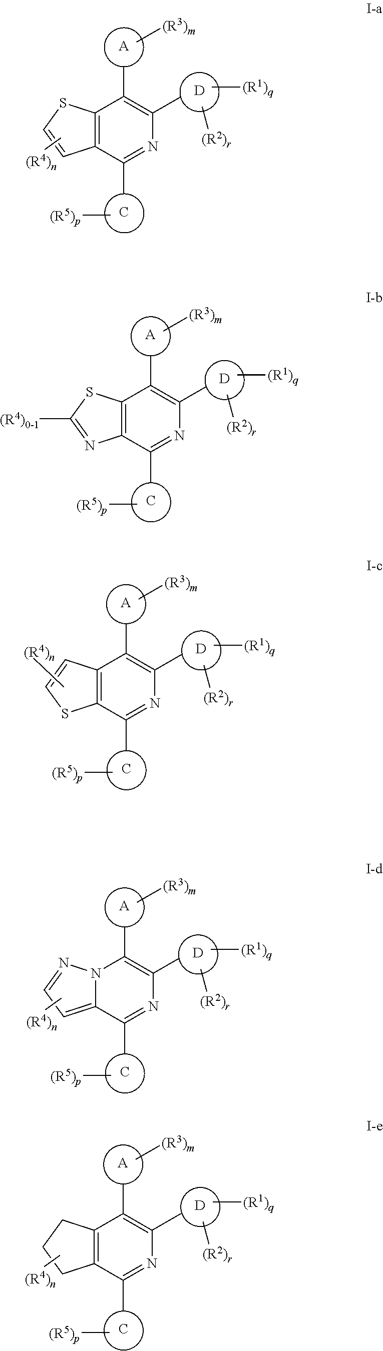 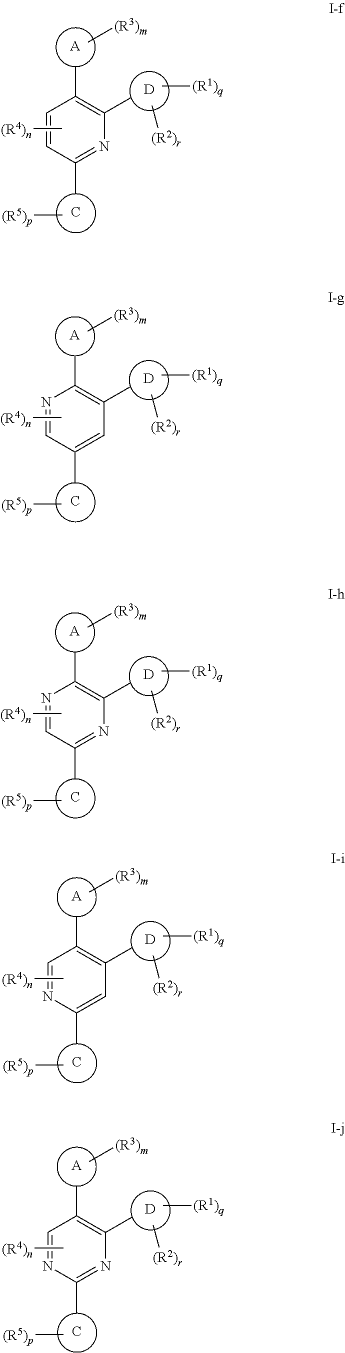 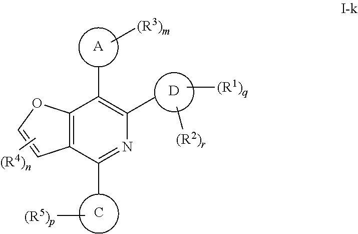
or a salt (e.g., pharmaceutically acceptable salt) thereof.

15. The compound of claim 1, wherein the compound is a compound according to one of Formulae I-a-i, I-b-i, I-c-i, I-d-i, I-e-i, I-f-i, I-g-i, I-h-i, I-i-i, I-j-i, or I-k-i: 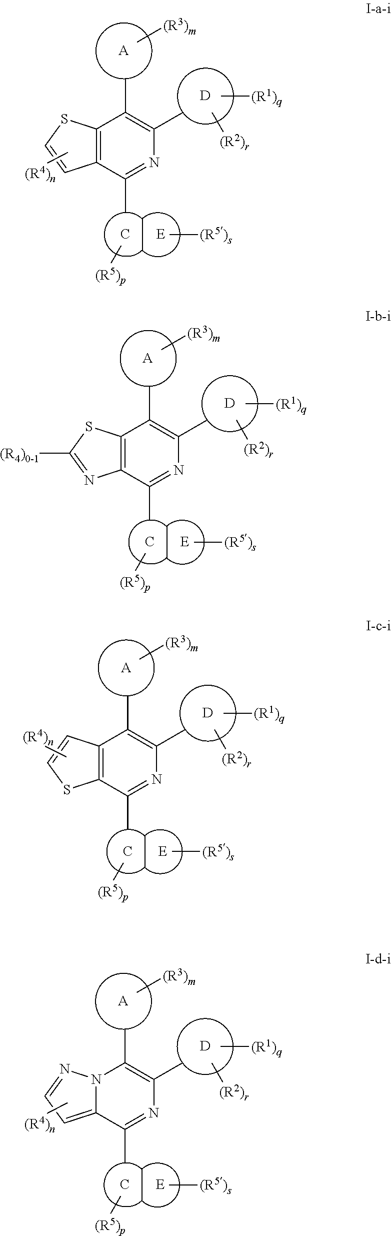 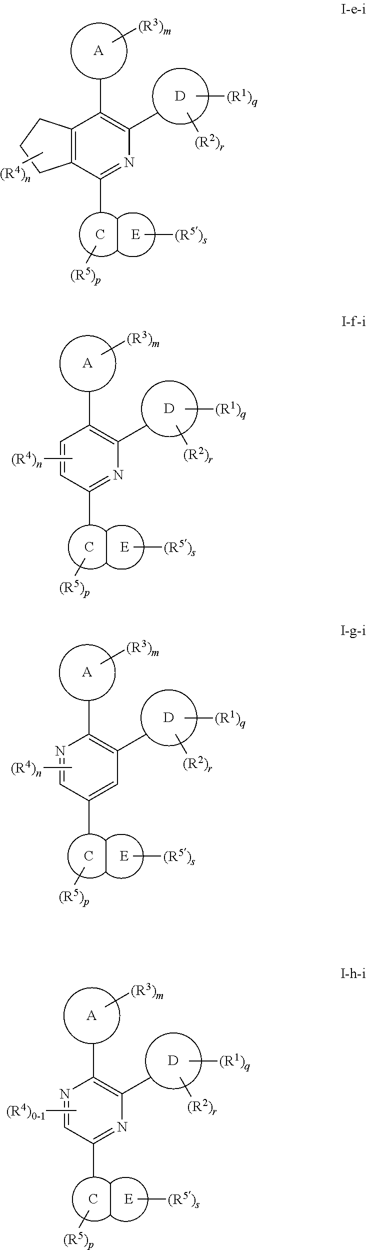 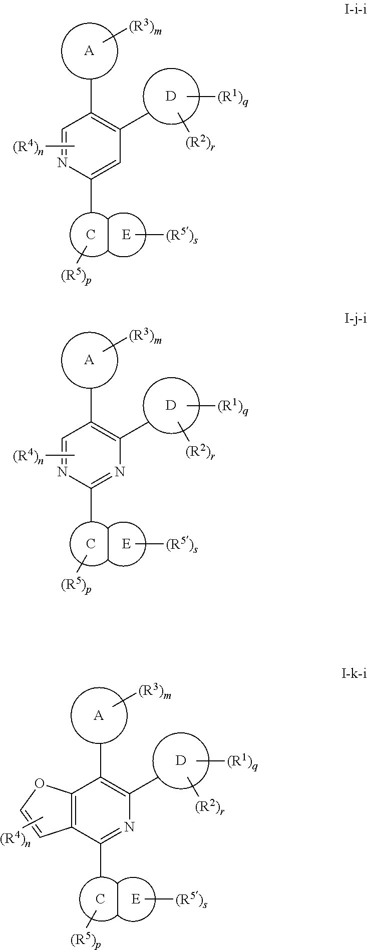
or a salt (e.g., pharmaceutically acceptable salt) thereof.

16. The compound of claim 1, wherein the compound is a compound according to one of Formulae I-a-ii′, I-b-ii′, I-c-ii′, I-d-ii′, I-e-ii′, I-f-ii′, I-g-ii′, I-h-ii′, I-i-ii′, I-j-ii′, or I-k-ii′: 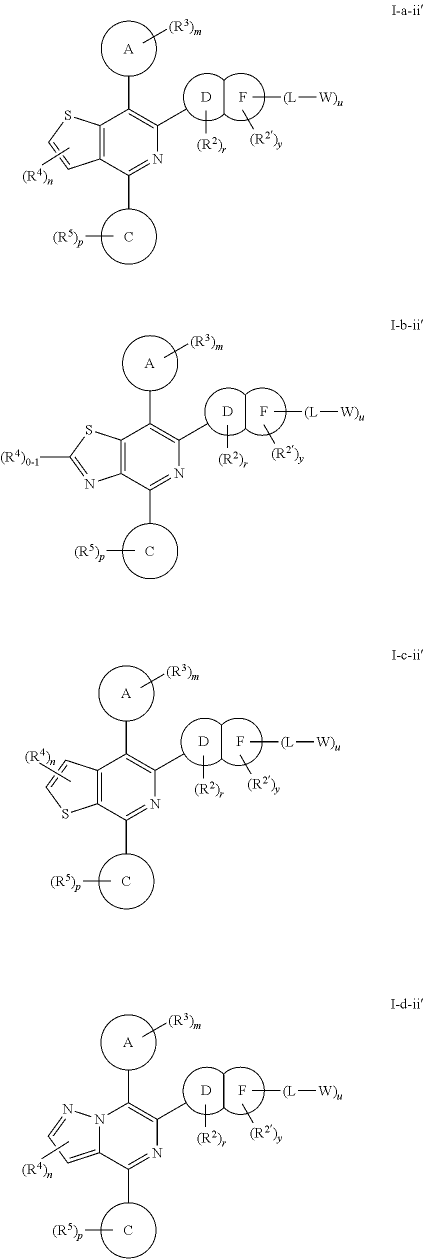 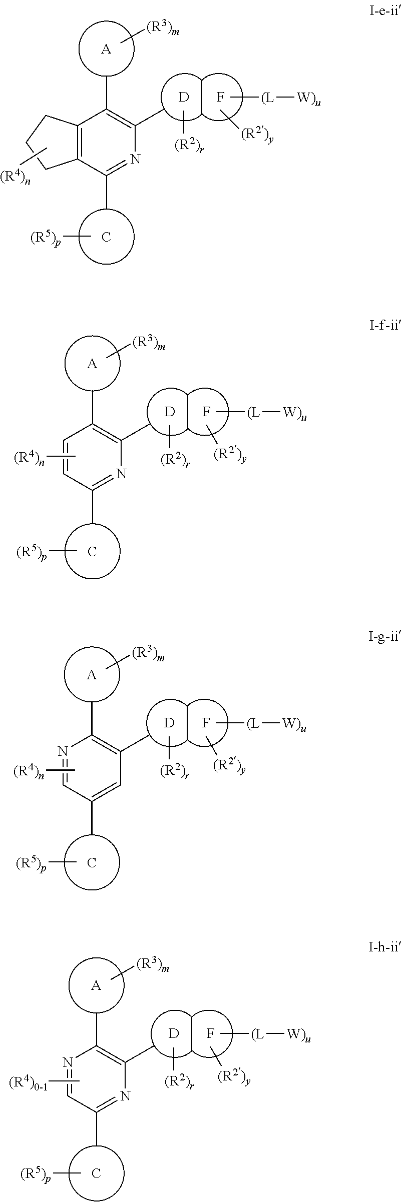 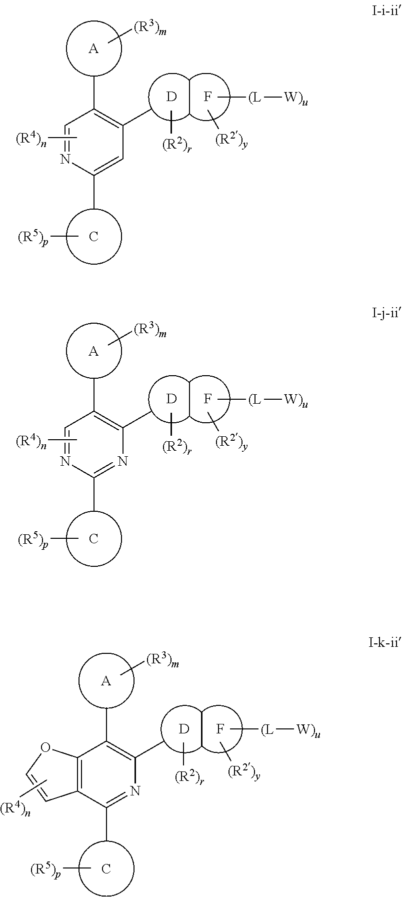
or a salt (e.g., pharmaceutically acceptable salt) thereof.

17. The compound of claim 1, wherein the compound is a compound according to one of Formulae I-a-iii, I-b-iii, I-c-iii, I-d-iii, I-e-iii, I-g-iii, I-h-iii, I-i-iii, I-j-iii, or I-k-iii: 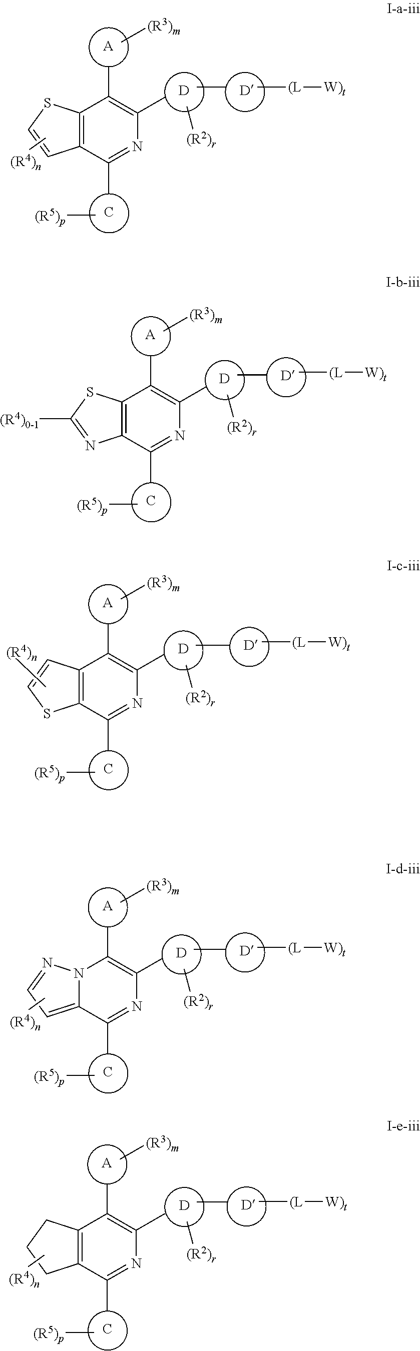 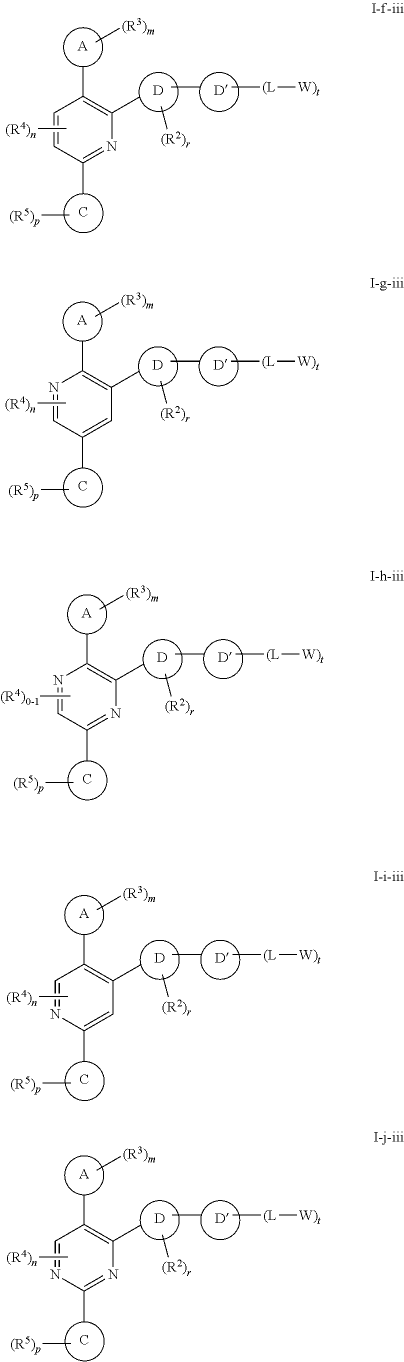 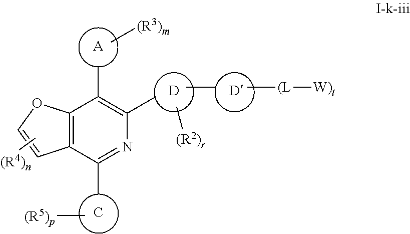
or a salt (e.g., pharmaceutically acceptable salt) thereof.

18. The compound of claim 1, wherein the compound is a compound according to one of Formulae I-a-iv, I-b-iv, I-c-iv, I-d-iv, I-e-iv, I-f-iv, I-g-iv, I-h-iv, I-i-iv, I-j-iv, or I-k-iv: 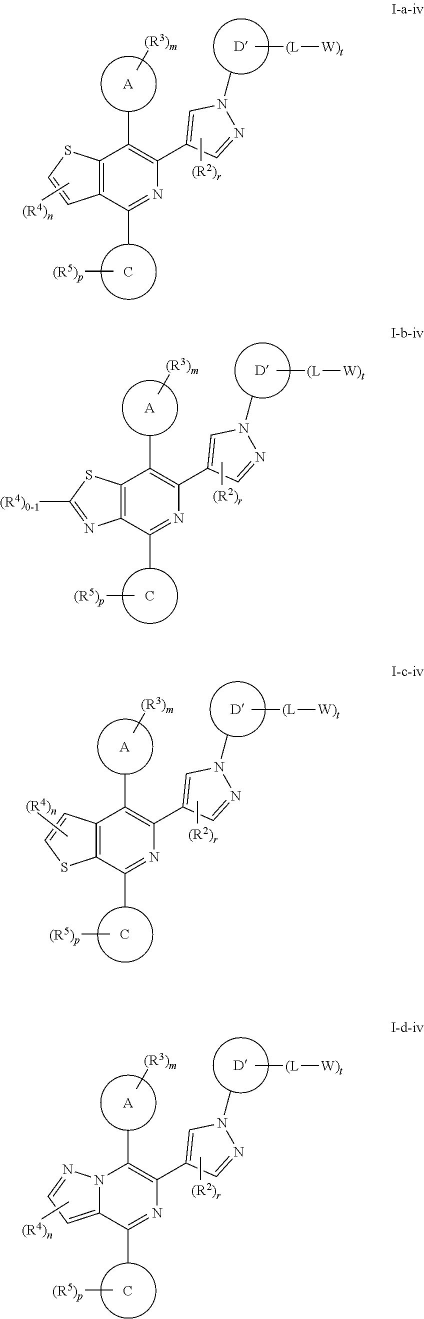 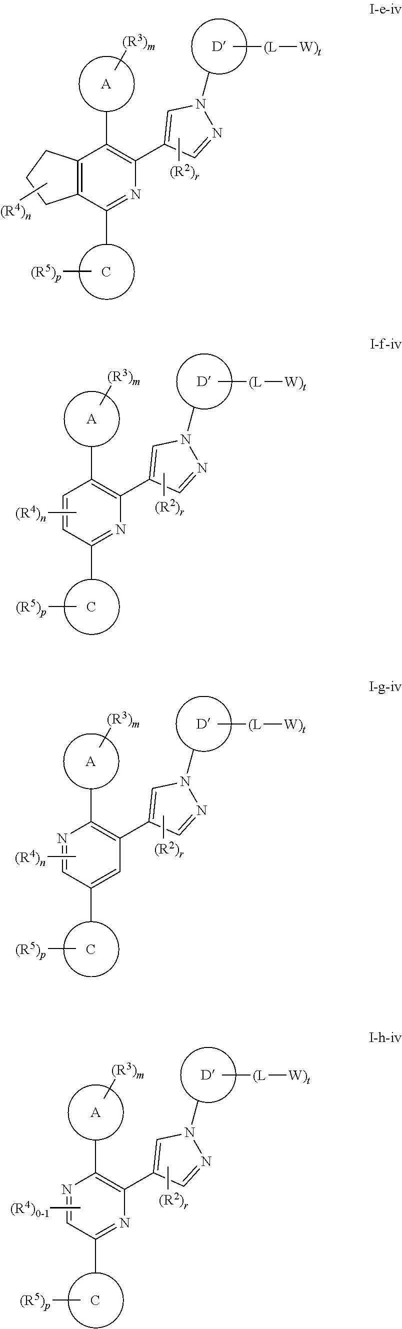 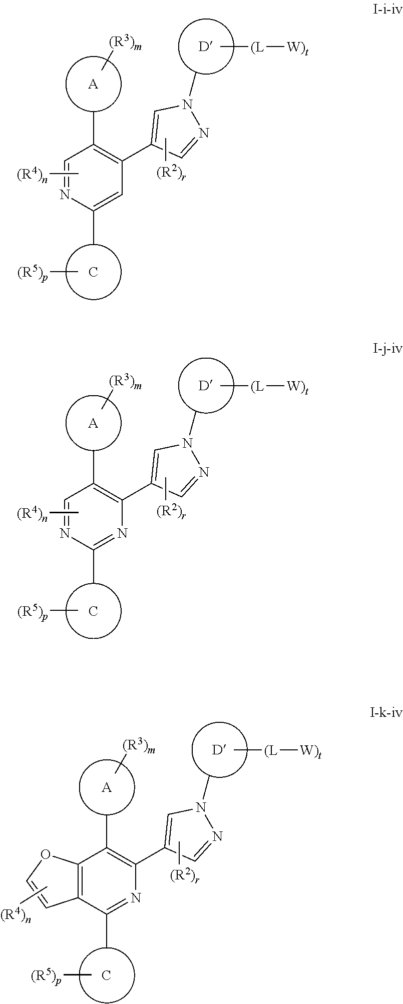
or a salt (e.g., pharmaceutically acceptable salt) thereof.

19. The compound of claim 1, wherein the compound is a compound according to one of Formulae I-a-v′, I-b-v′, I-c-v′, I-d-v′, I-e-v′, I-f-v′, I-g-v′, I-h-v′, I-i-v′, I-j-v′, or I-k-v′: 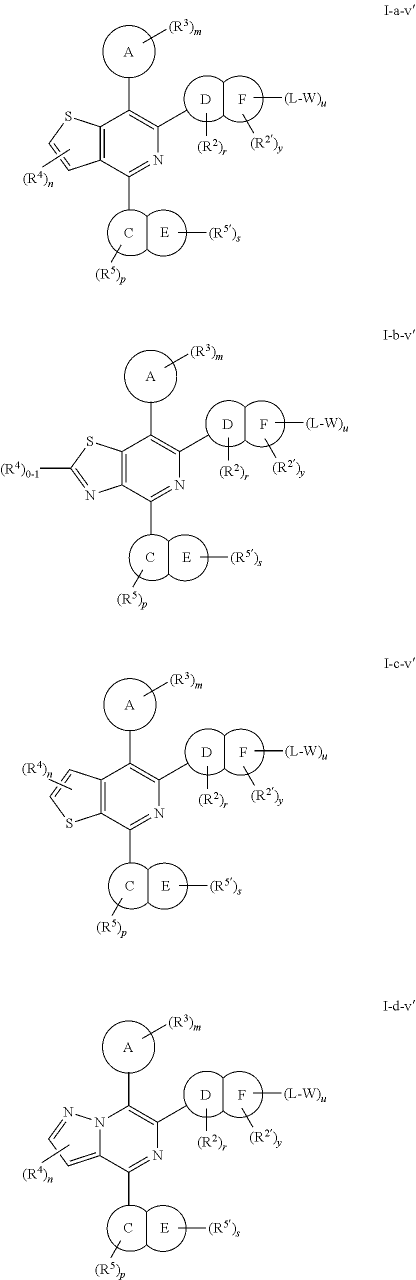 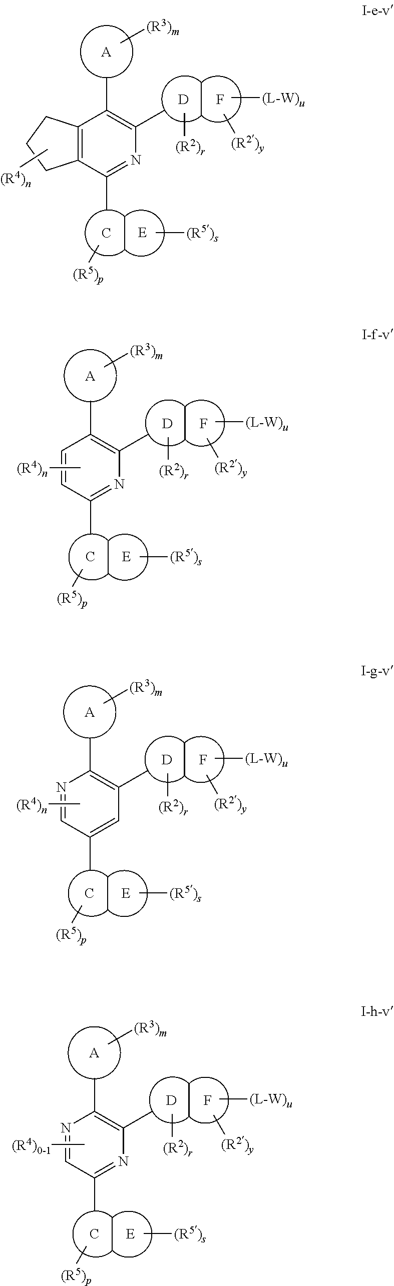 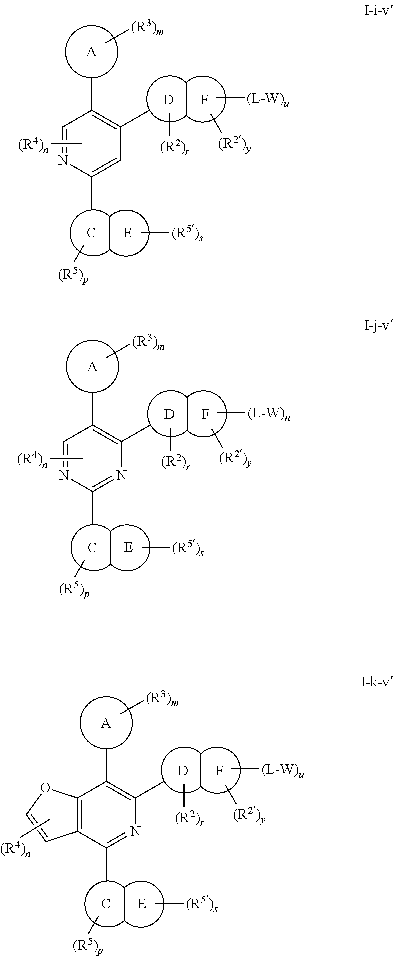
or a salt (e.g., pharmaceutically acceptable salt) thereof.

20. The compound of claim 1, wherein the compound is a compound according to one of Formulae I-l, I-m, I-n, I-p, I-q, or I-r: 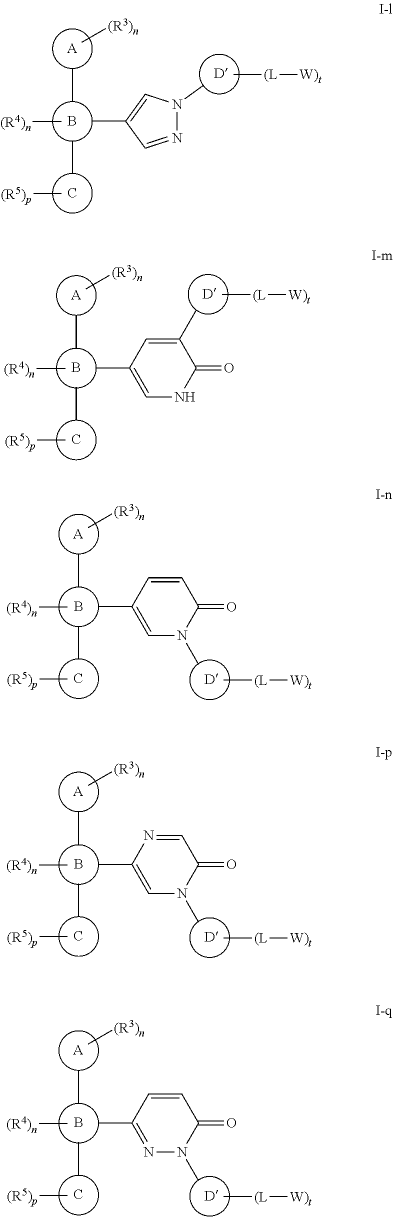 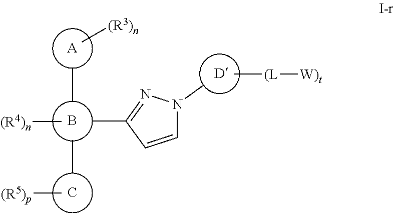
or a salt (e.g., pharmaceutically acceptable salt) thereof.

21. The compound of any one of claims 1-20, wherein Ring A is phenyl.

22. The compound of claim 21, wherein Ring A is

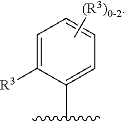

23. The compound of claim 22, wherein Ring A is selected from

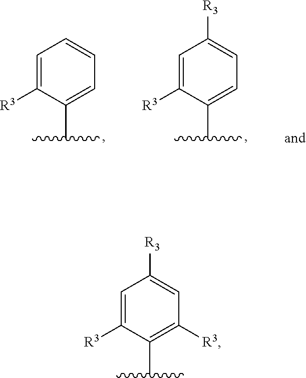

24. The compound of claim 23, wherein Ring A is selected from

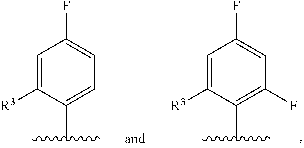

25. The compound of any one of claims 21-24, wherein Ring A, substituted with m instances of R³, is selected from

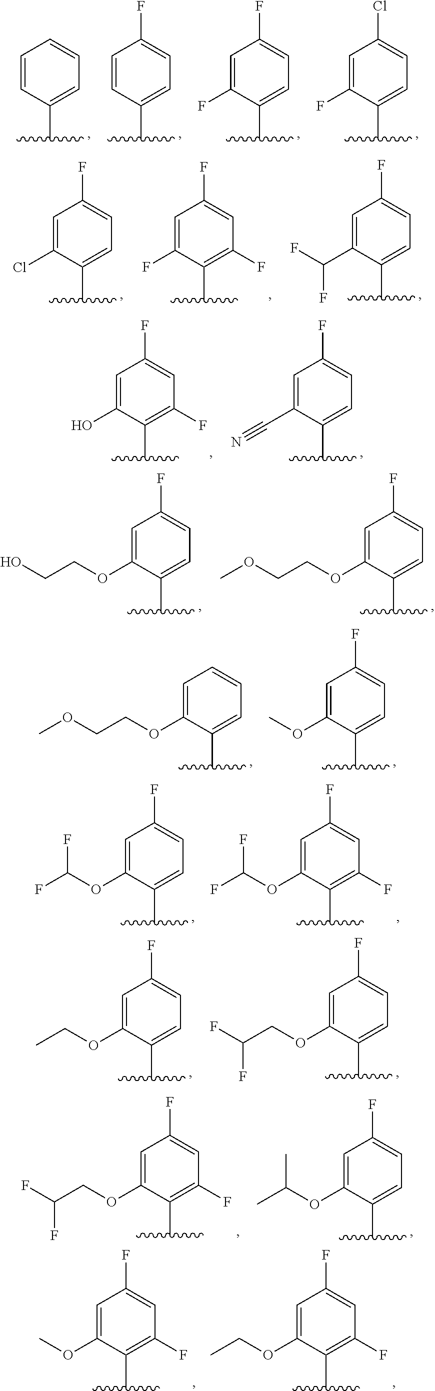

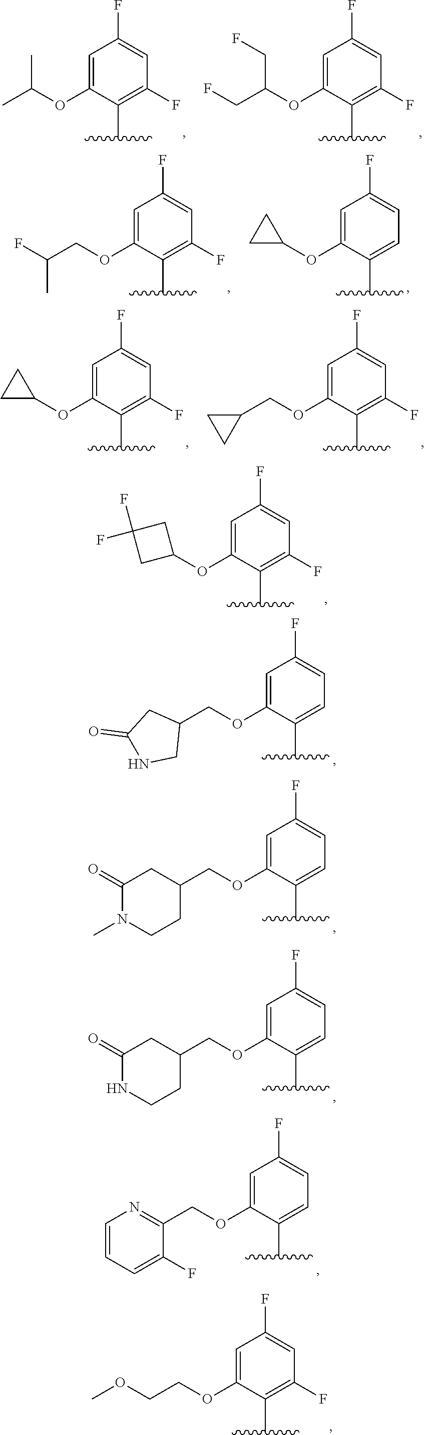

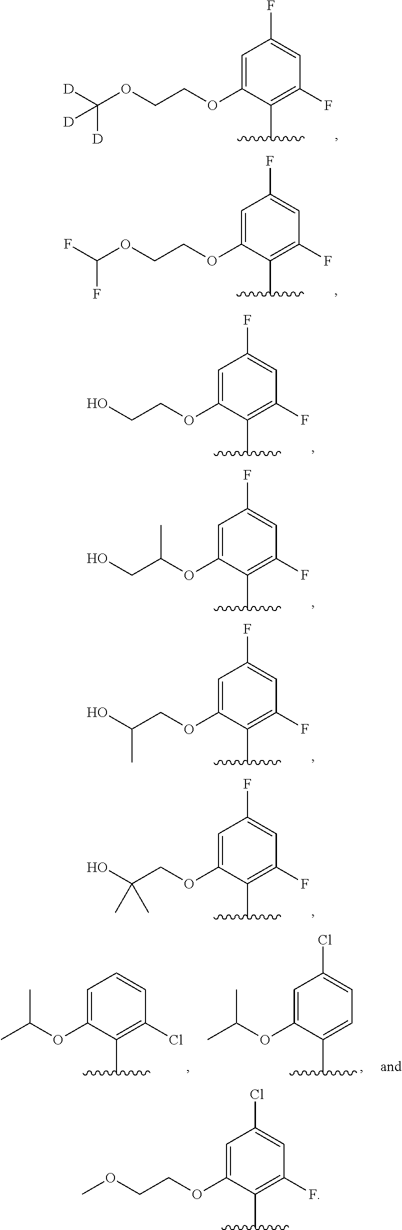

26. The compound of any one of claims 1-20, wherein Ring A is a 5- to 6-membered heteroaryl ring having 1-3 heteroatoms independently selected from nitrogen, oxygen, and sulfur.

27. The compound of claim 26, wherein Ring A substituted with m instances of R³ is selected from

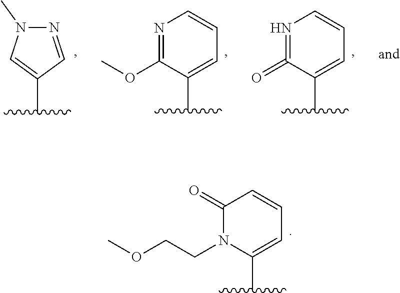

28. The compound of any one of claims 1-27, wherein m is 1, 2, or 3.

29. The compound of any one of claims 1-28, wherein at least one R³ is selected from halogen, —OR, —O(CH₂)ᵥCy, and —O—(C₁₋₄ alkylene)-OR.

30. The compound of any one of claims 1-29, wherein Ring B is selected from a 9- to 10-membered bicyclic ring that comprises at least one 5- or 6-membered heteroaryl ring comprising at least one nitrogen atom.

31. The compound of claim 30, wherein Ring B, substituted with n instances of R⁴, is selected from

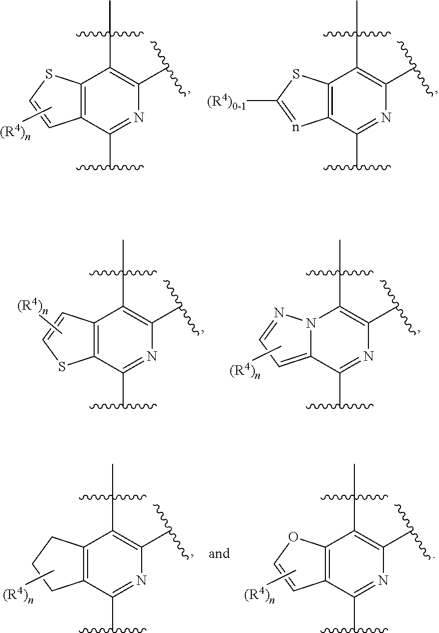

32. The compound of any one of claims 1-29, wherein Ring B is selected from a 6-membered heteroaryl ring having 1-2 nitrogen atoms.

33. The compound of claim 32, wherein Ring B, substituted with n instances of R⁴, is selected from:

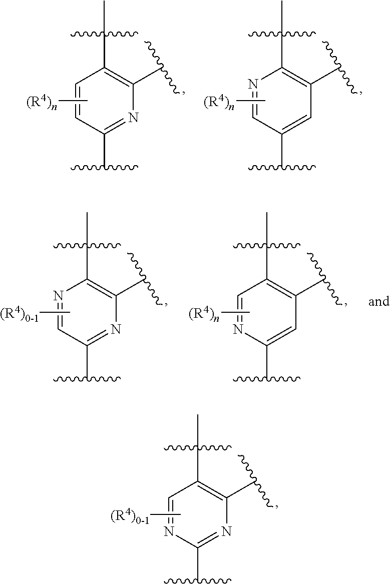

34. The compound of any one of claims 1-29, wherein Ring B is phenyl.

35. The compound of any one of claims 1-34, wherein each R⁴ is halogen.

36. The compound of any one of claims 1-35, wherein n is 0.

37. The compound of any one of claims 1-36, wherein Ring C is phenyl.

38. The compound of any one of claims 1-36, wherein Ring C is selected from a 5- to 6-membered heteroaryl ring having 1-3 heteroatoms independently selected from nitrogen, oxygen, and sulfur.

39. The compound of any one of claims 1-36, wherein Ring C is selected from a 5- to 8-membered bicyclic carbocyclic ring.

40. The compound of any one of claims 1-36, wherein Ring C is selected from a 4- to 8-membered heterocyclic ring having 1-2 heteroatoms independently selected from nitrogen, oxygen, and sulfur.

41. The compound of any one of claims 1-36, wherein Ring C is selected from a 7- to 10-membered spirofused heterocyclic ring having 1-3 heteroatoms independently selected from nitrogen, oxygen, and sulfur.

42. The compound of any one of claims 1-41, wherein Ring C is not fused to Ring E, and Ring C, substituted with p instances of R⁵, is selected from:

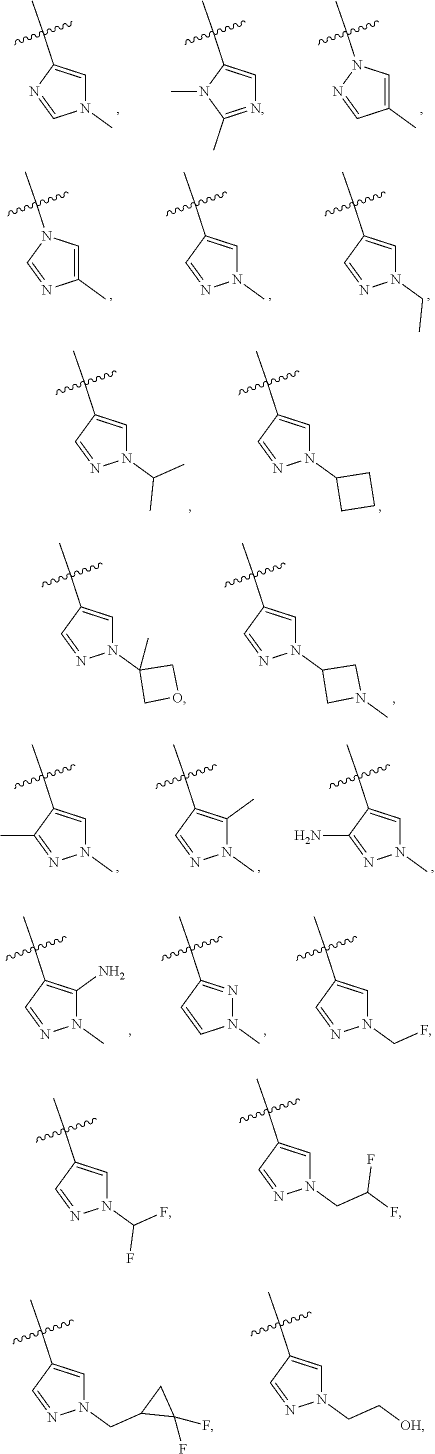

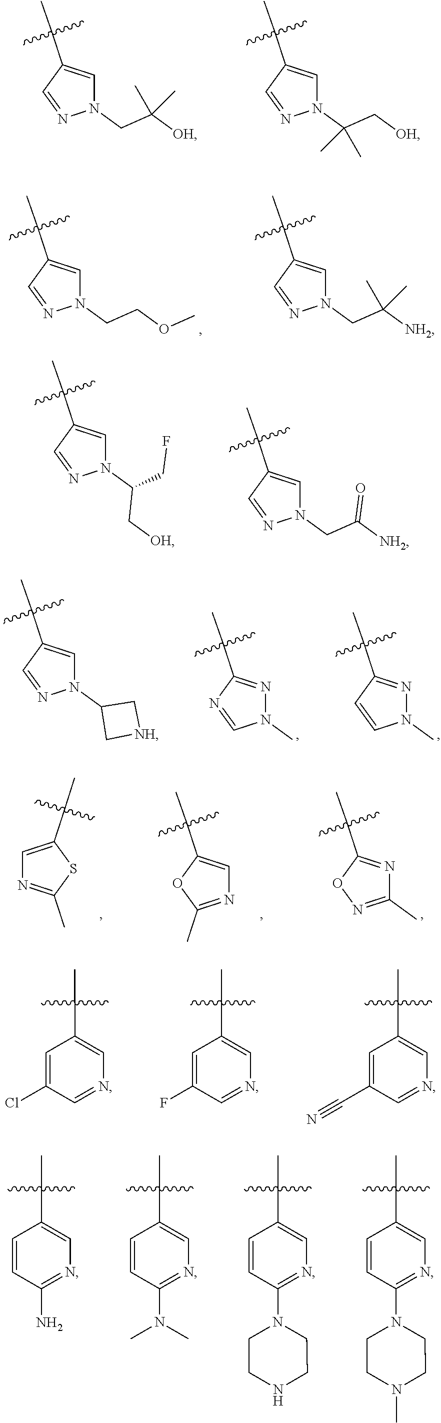

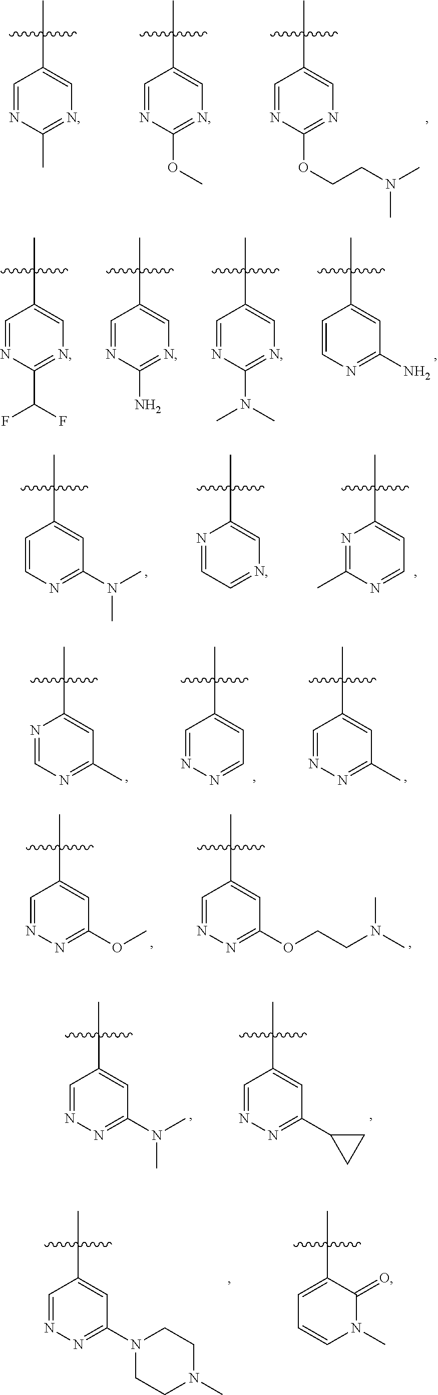

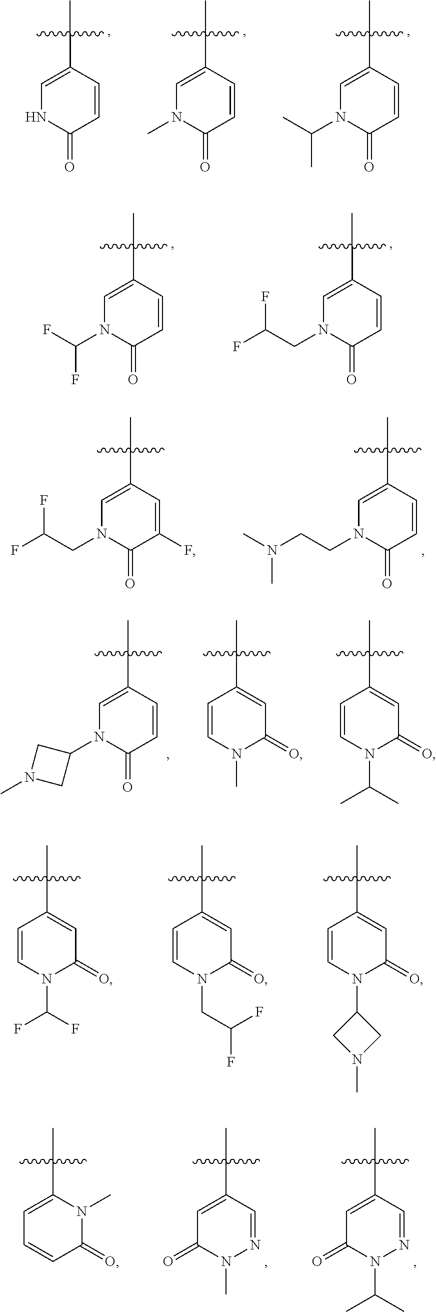

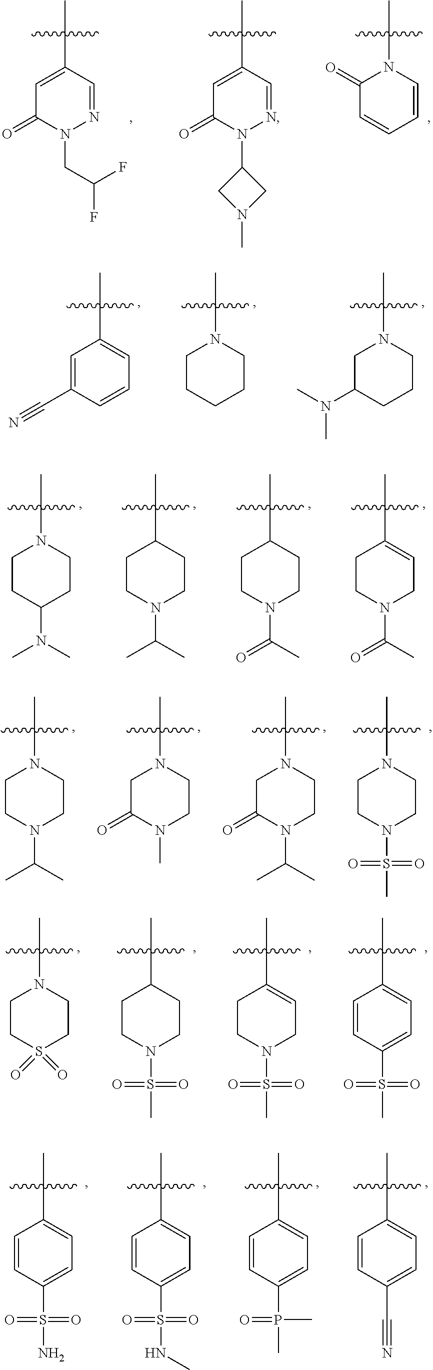

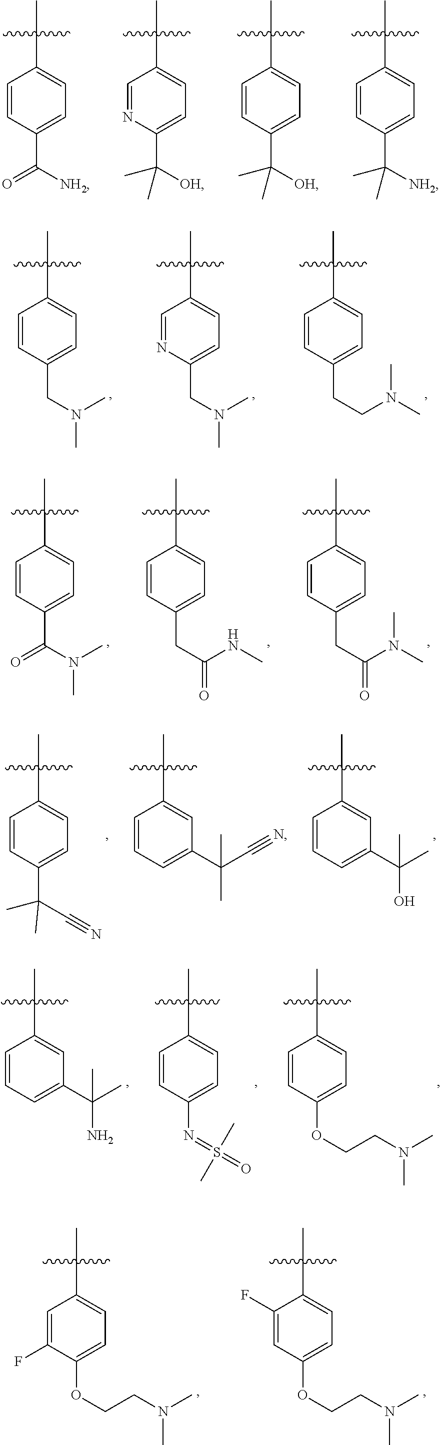

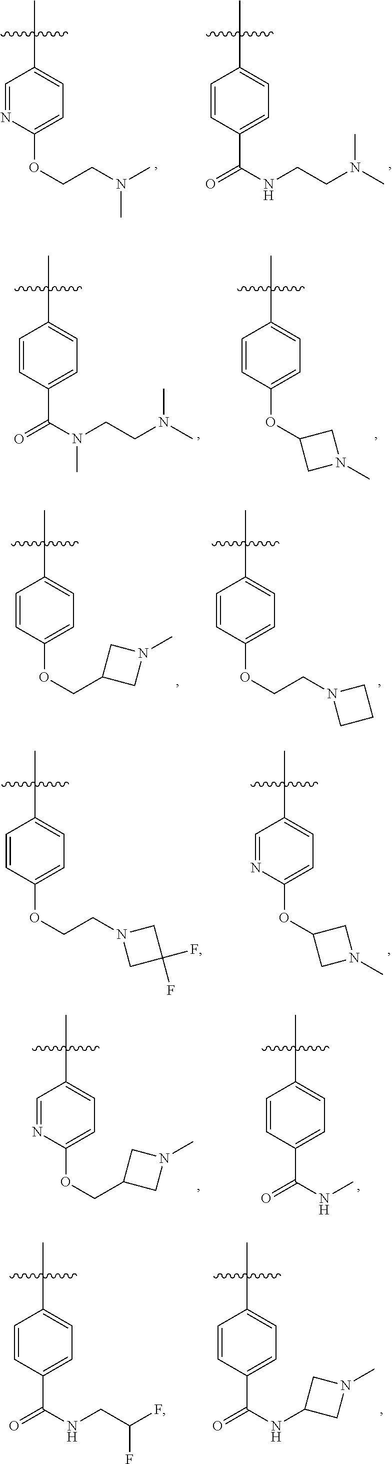

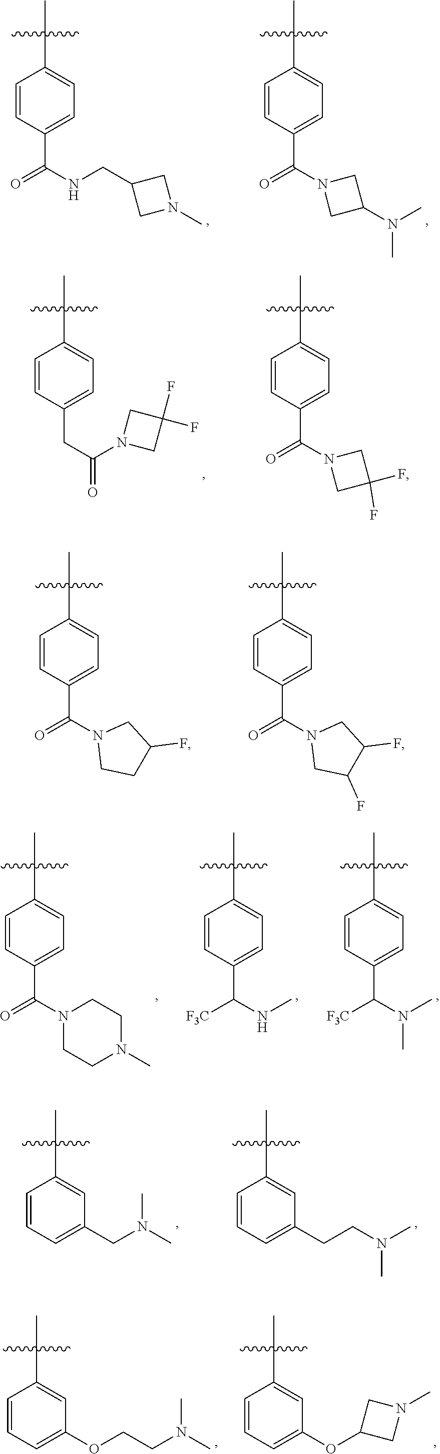

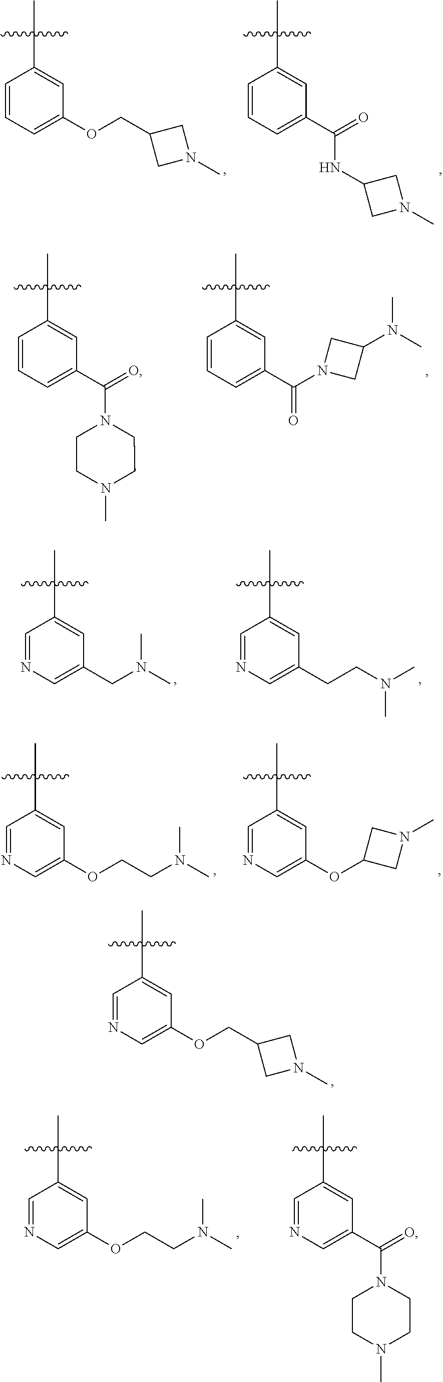

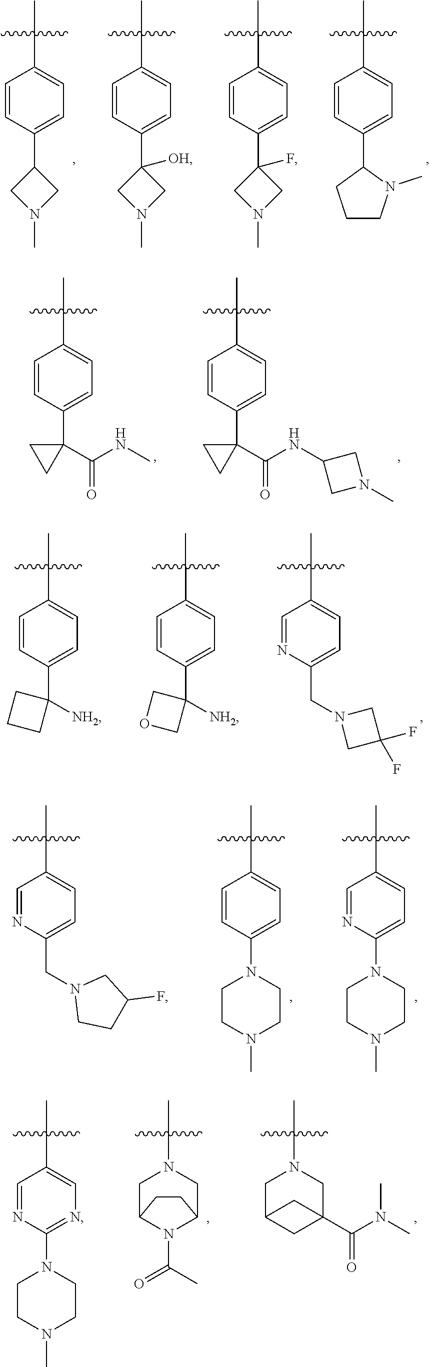

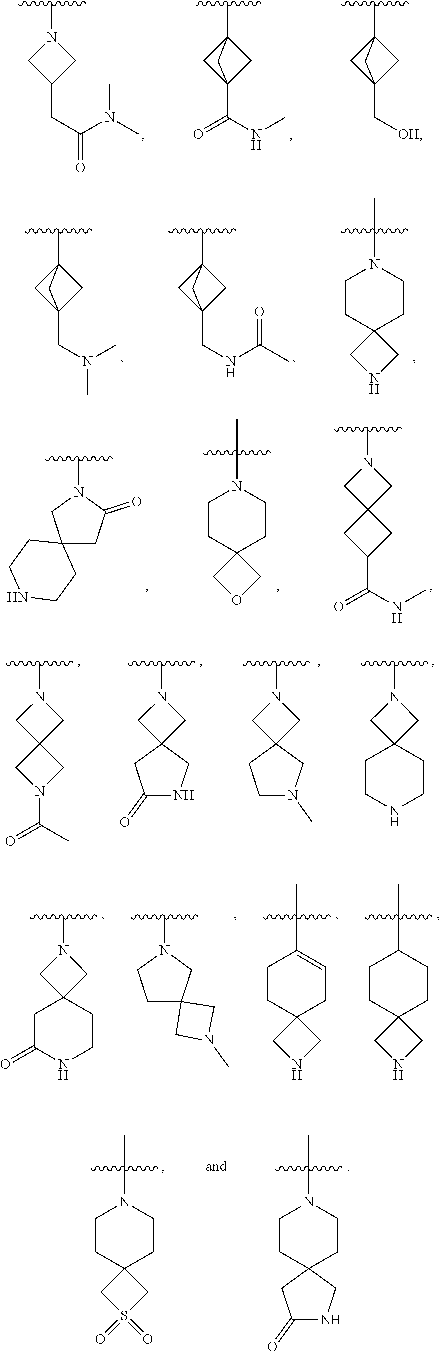

43. The compound of any one of claims 1-41, wherein Ring C is fused to Ring E.

44. The compound of claim 43, wherein 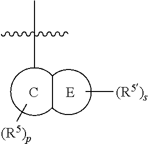
is selected from

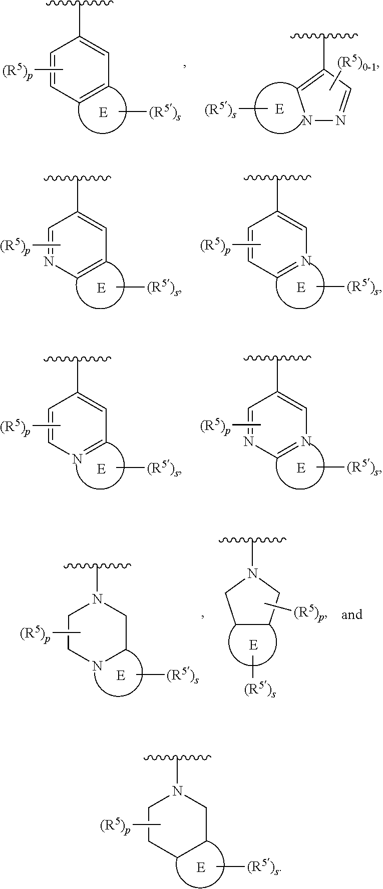

45. The compound of claim 43 or 44, wherein Ring E is selected from a 5- to 6-membered carbocyclic ring, wherein Ring E is substituted by s instances of R^{5′}.

46. The compound of claim 43 or 44, wherein Ring E is selected from a 5- to 7-membered heterocyclic ring having 1-2 heteroatoms independently selected from nitrogen, oxygen, and sulfur, wherein Ring E is substituted by s instances of R^{5′}.

47. The compound of claim 43 or 44, wherein Ring E is selected from a 7- to 10-membered spirofused heterocyclic ring having 1-3 heteroatoms independently selected from nitrogen, oxygen, and sulfur, wherein Ring E is substituted by s instances of R^{5′}.

48. The compound of claim 43 or 44, wherein Ring E is selected from a 5- to 6-membered heteroaryl ring having 1-3 heteroatoms independently selected from nitrogen, oxygen, and sulfur, wherein Ring E is substituted by s instances of R^{5′}.

49. The compound of any one of claims 43-48, wherein Ring C is fused to Ring E, and Ring E, substituted with s instances of R^{5′}, is selected from:

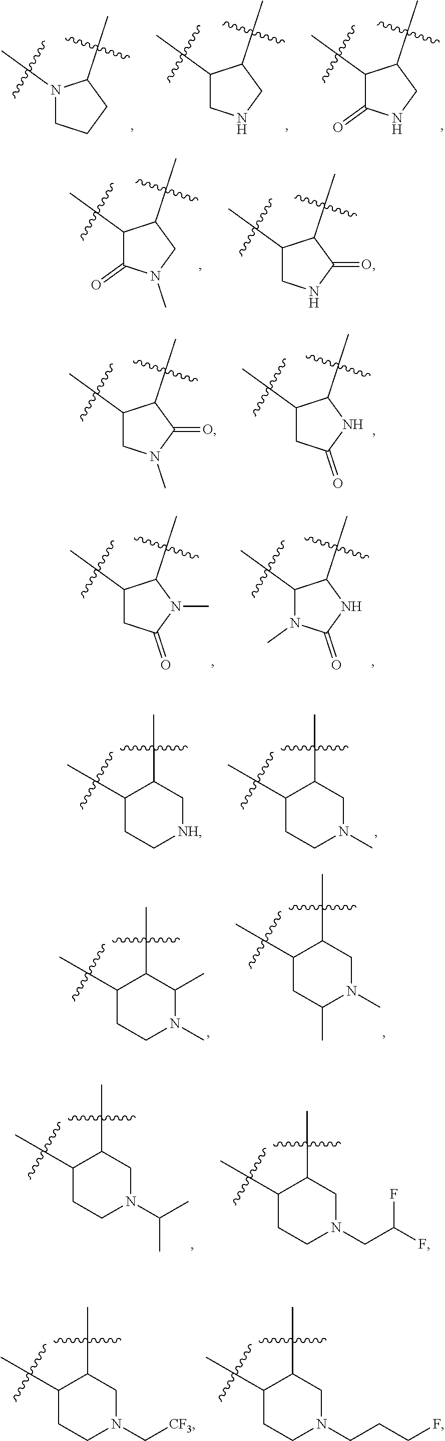

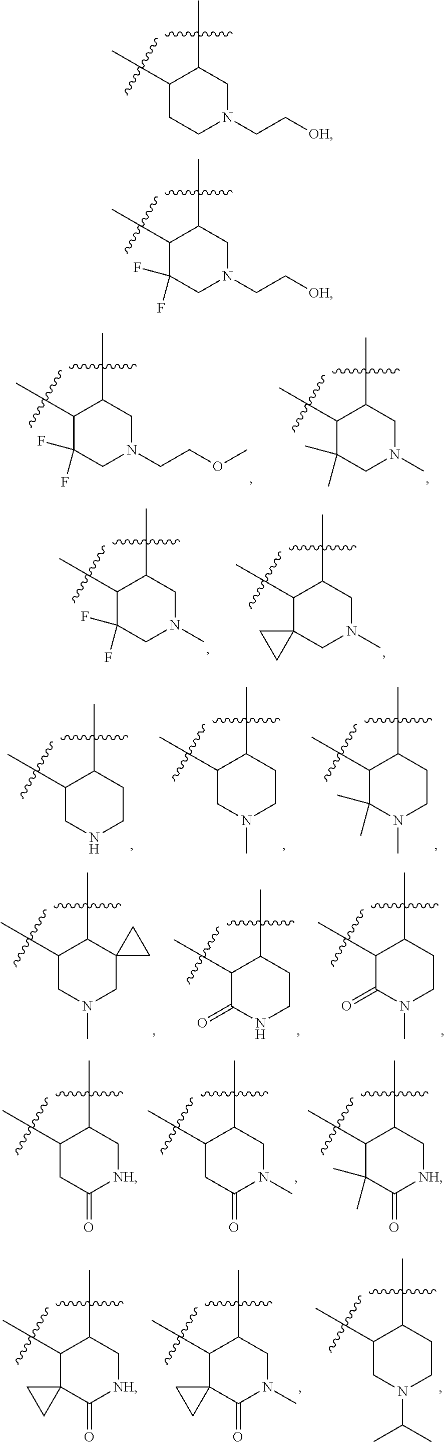

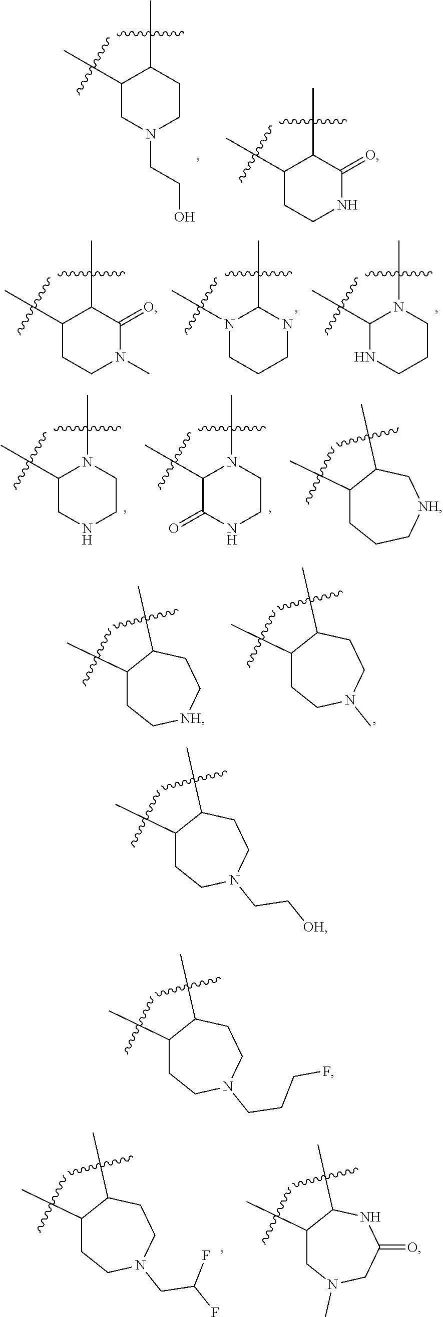

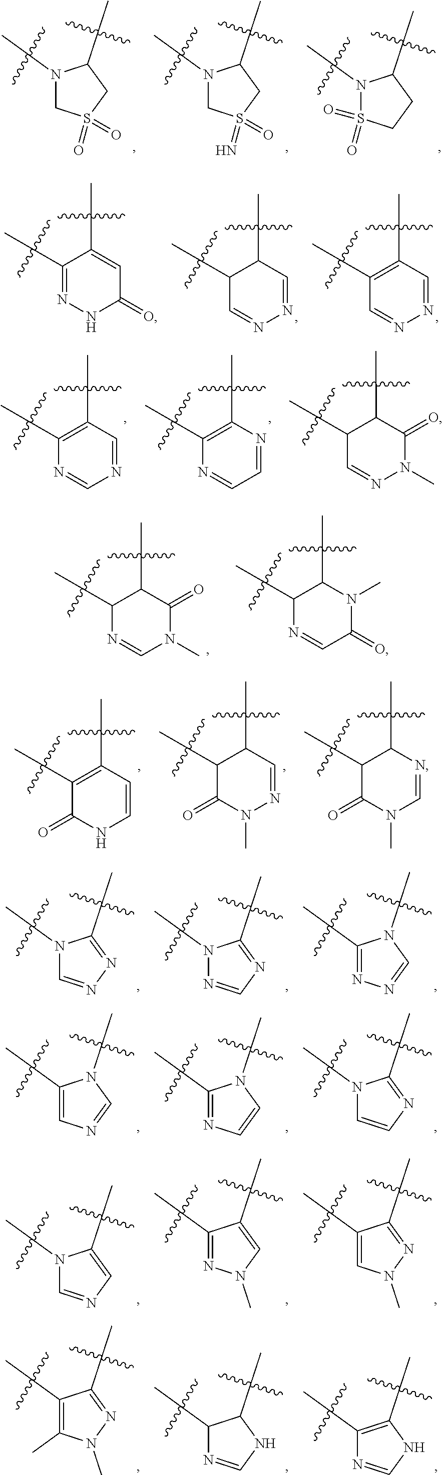

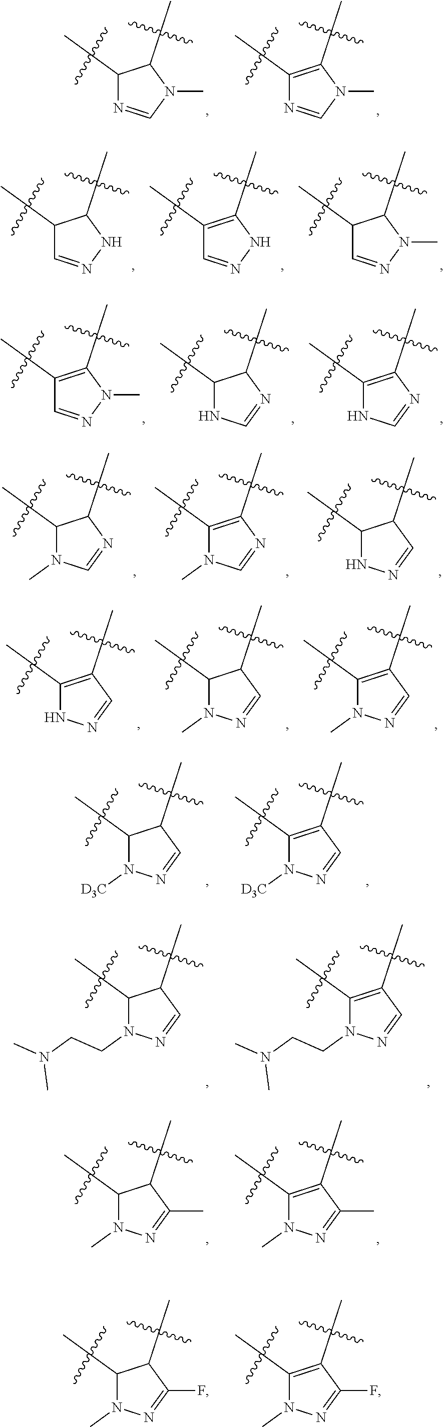

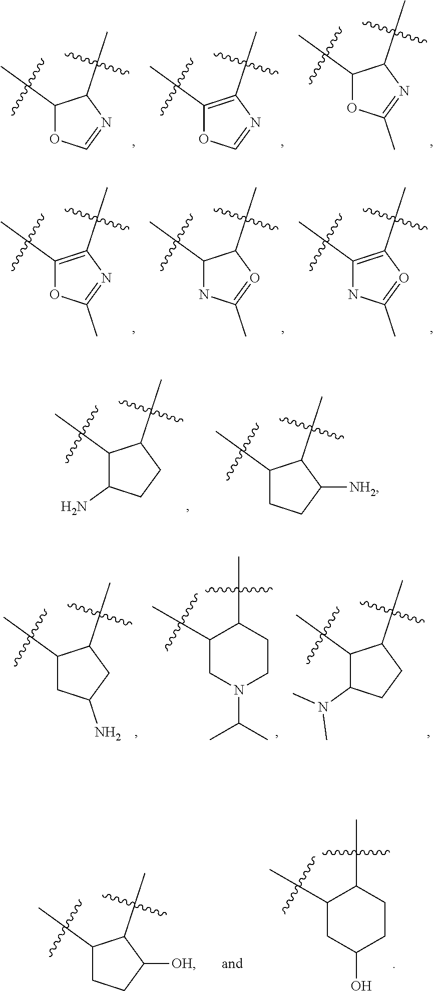

50. The compound of any one of claims 43-49, wherein 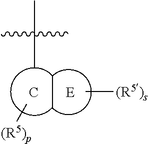
is selected from:

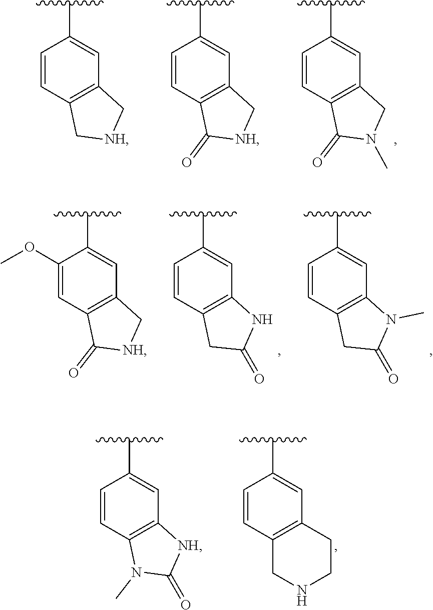

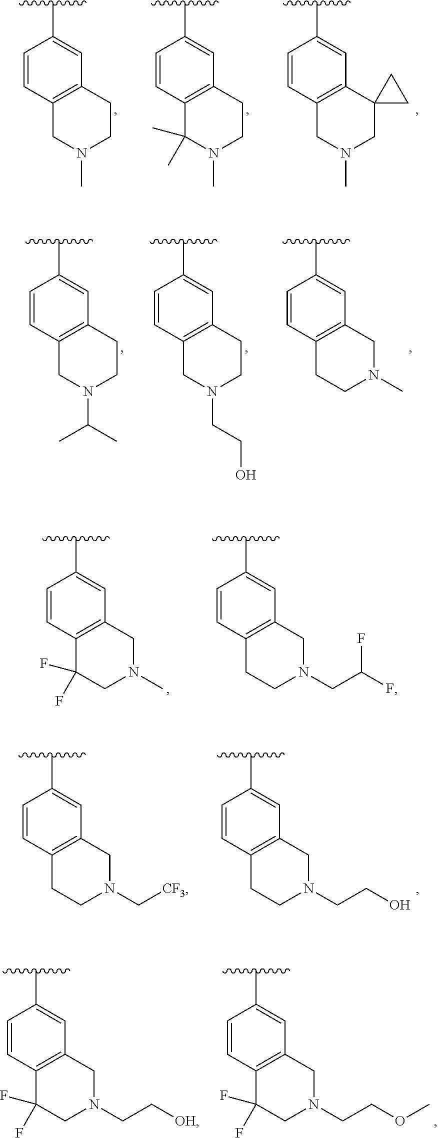

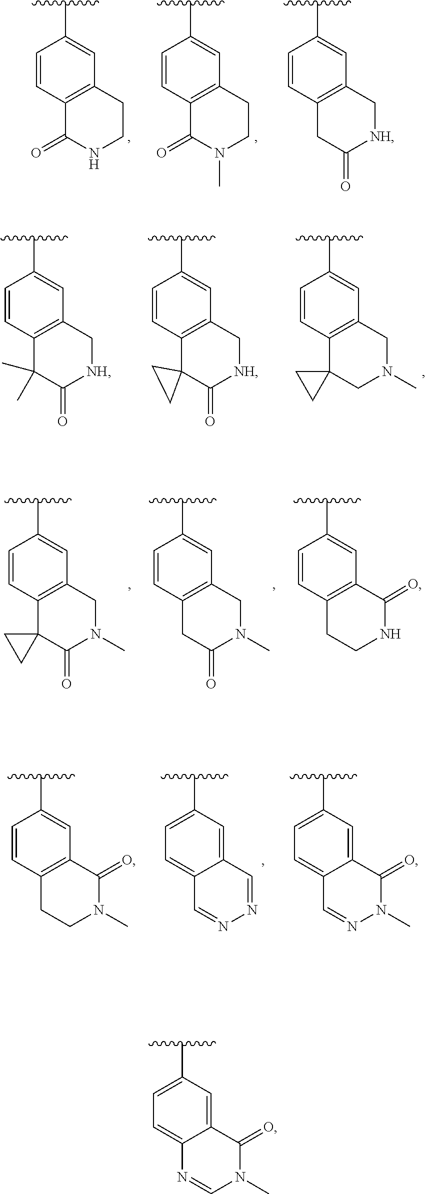

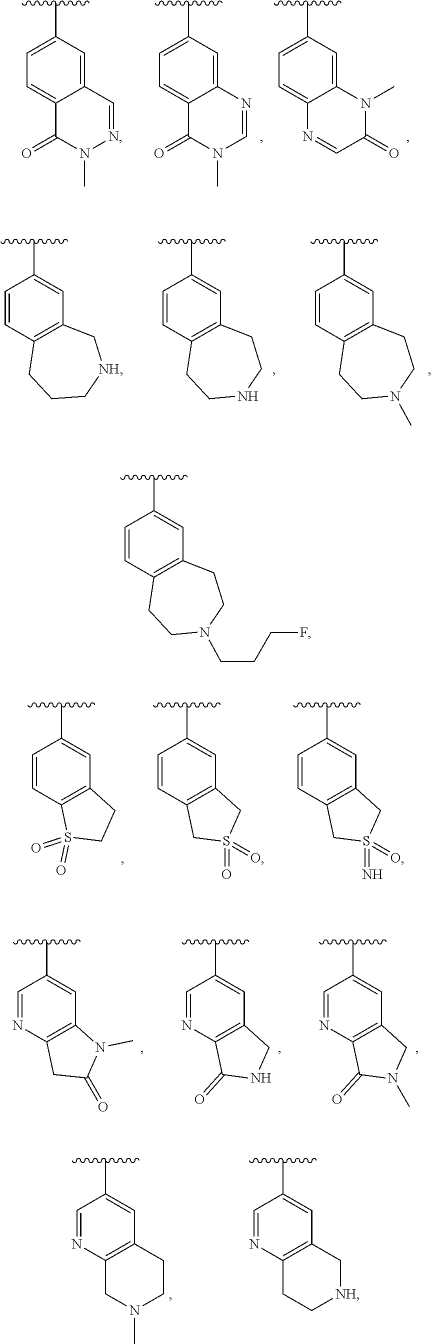

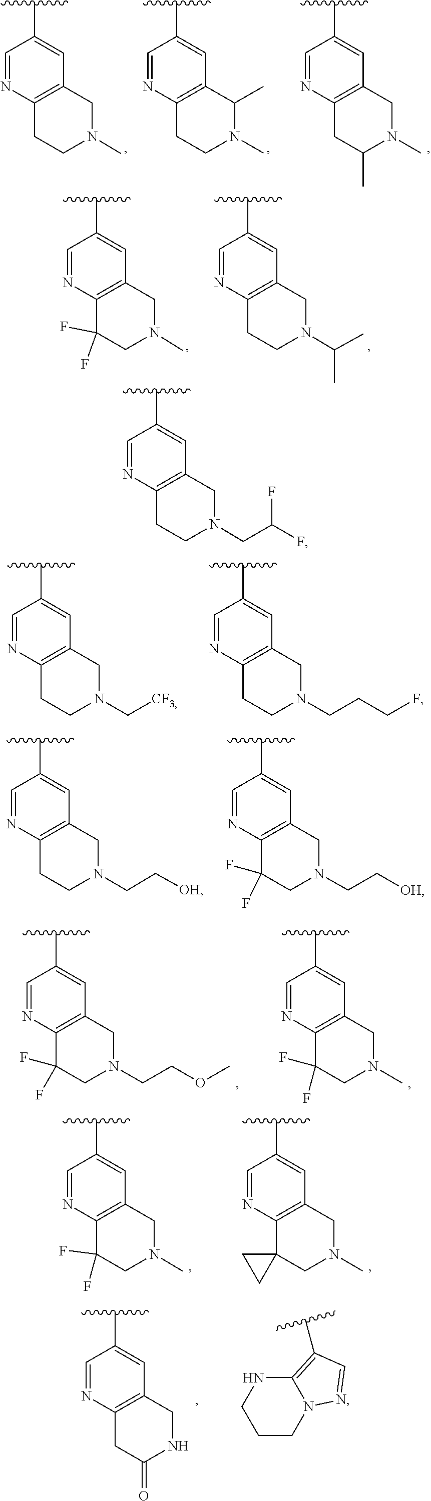

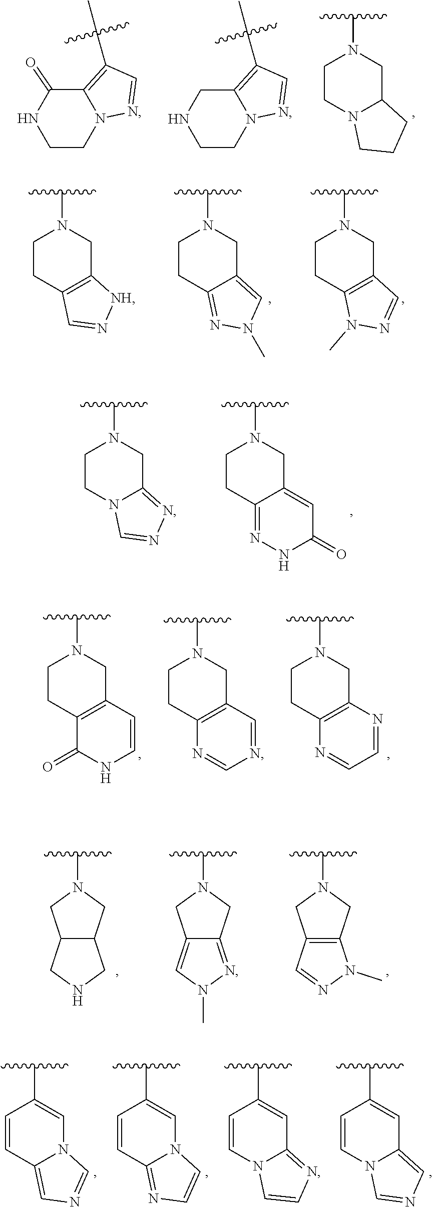

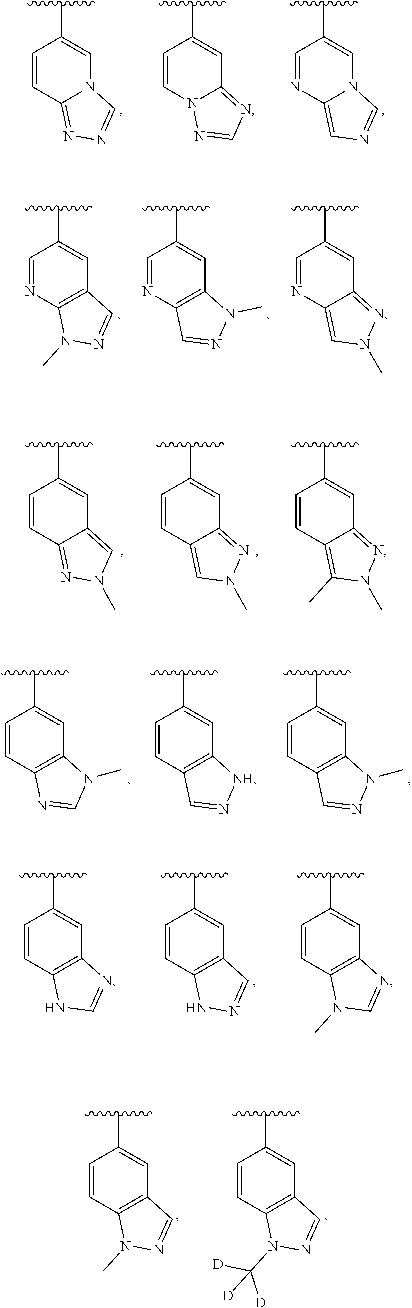

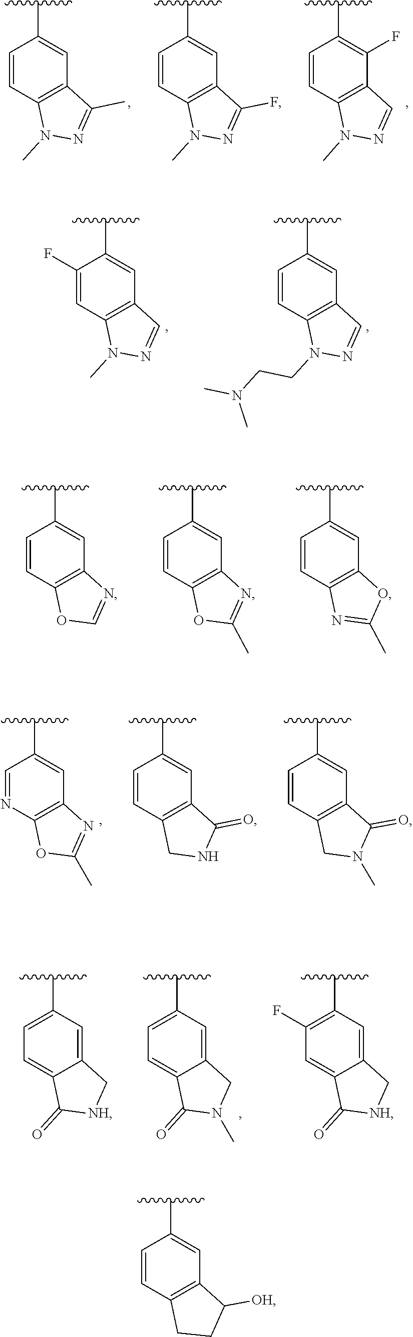

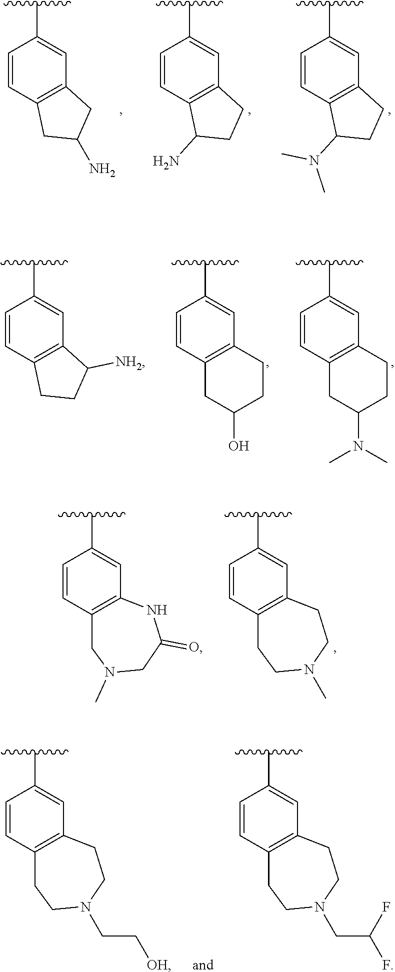

51. The compound of any one of claims 1-50, wherein each R⁵ is independently selected from oxo, =NH, —CN, halogen, —OR, —N(R)₂, —SR, —C(O)R, —N(R)C(O)R, —(CH₂)ₓC(O)N(R)₂, —C(O)N(R)(CH₂)ₓCy, —(CH₂)ₓC(O)Cy, —OC(O)R, —C(O)OR, —SO₂R, —N(R)SO₂R, —N═S(O)(R)₂, —SO₂N(R)₂, —P(O)(R)₂, —(CH₂)ₓCy, —O(CH₂)ₓCy, and C₁₋₆ aliphatic, wherein C₁₋₆ aliphatic is unsubstituted or substituted with one or more halogen, —CN, —N(R)C(O)R, —N(R)₂, or —OR.

52. The compound of any one of claims 1-51, wherein p is 0.

53. The compound of any one of claims 1-51, wherein p is 1 or 2.

54. The compound of any one of claims 1-53, wherein each R^{5′} is independently selected from oxo, =NH, —CN, halogen, —OR, —N(R)₂, —SR, —C(O)R, —N(R)C(O)R, —(CH₂)ₓC(O)N(R)₂, —(CH₂)ₓC(O)Cy, —OC(O)R, —C(O)OR, —SO₂R, —N(R)SO₂R, —N═S(O)(R)₂, —SO₂N(R)₂, —P(O)(R)₂, —(CH₂)ₓCy, —O(CH₂)ₓCy, and C₁₋₆ aliphatic, wherein C₁₋₆ aliphatic is unsubstituted or substituted with one or more halogen, —CN, —N(R)C(O)R, —N(R)₂, or —OR.

55. The compound of any one of claims 1-54, wherein s is 0.

56. The compound of any one of claims 1-54, wherein s is 1 or 2.

57. The compound of any one of claims 1-56, wherein Ring D is phenyl.

58. The compound of any one of claims 1-56, wherein Ring D is a 5- to 6-membered heteroaryl ring having 1-3 heteroatoms independently selected from nitrogen, oxygen, and sulfur.

59. The compound of any one of claims 1-58, wherein Ring D is fused to Ring F.

60. The compound of claim 59, wherein q is 0.

61. The compound of claim 60, wherein 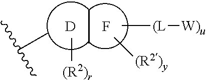
is selected from:

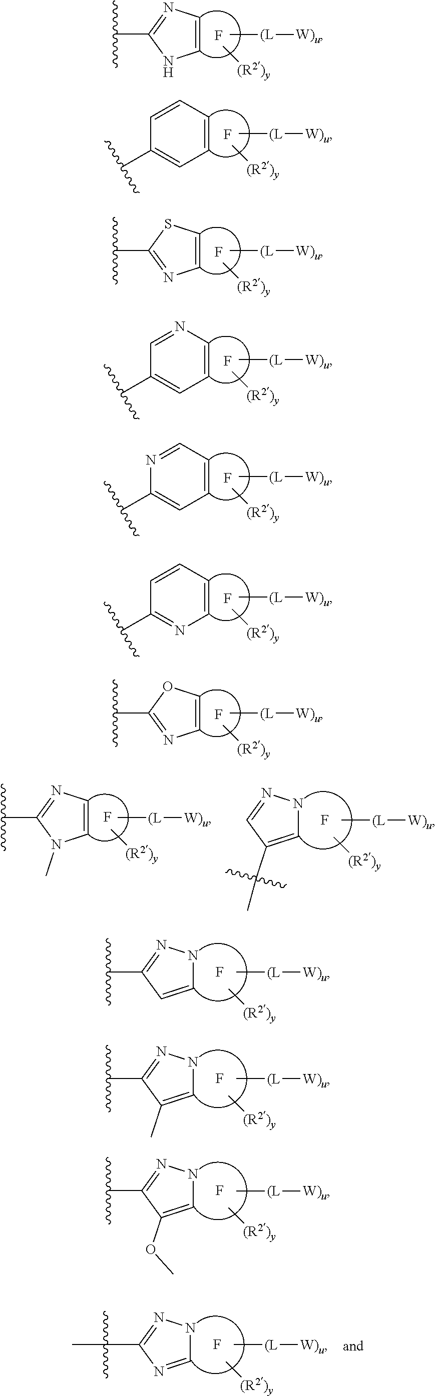

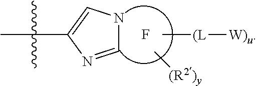

62. The compound of claim 60 or 61, wherein Ring F, substituted with u instances of -L-W and y instances of R^{2′}, is selected from:

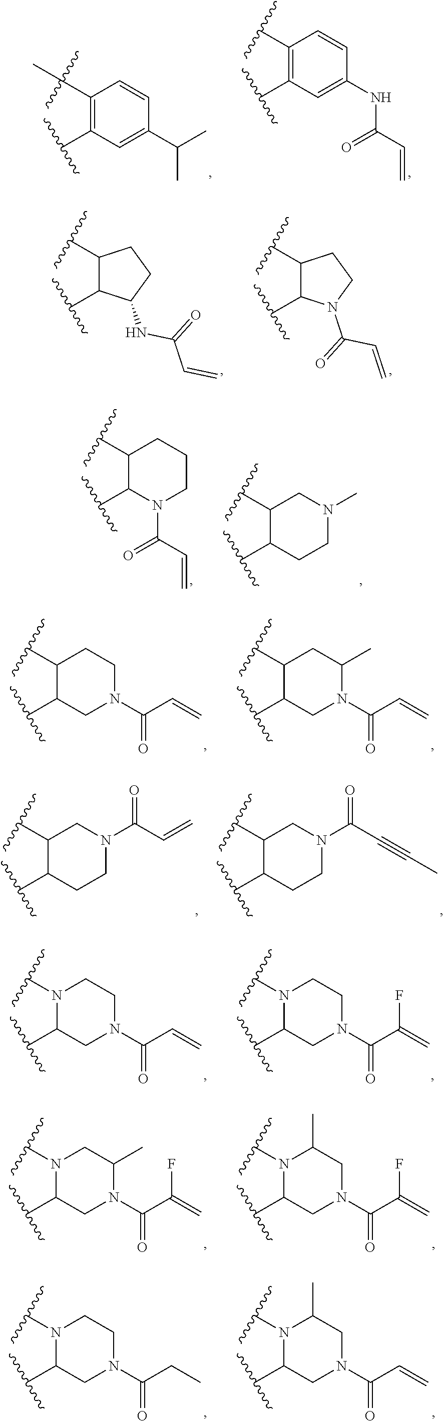

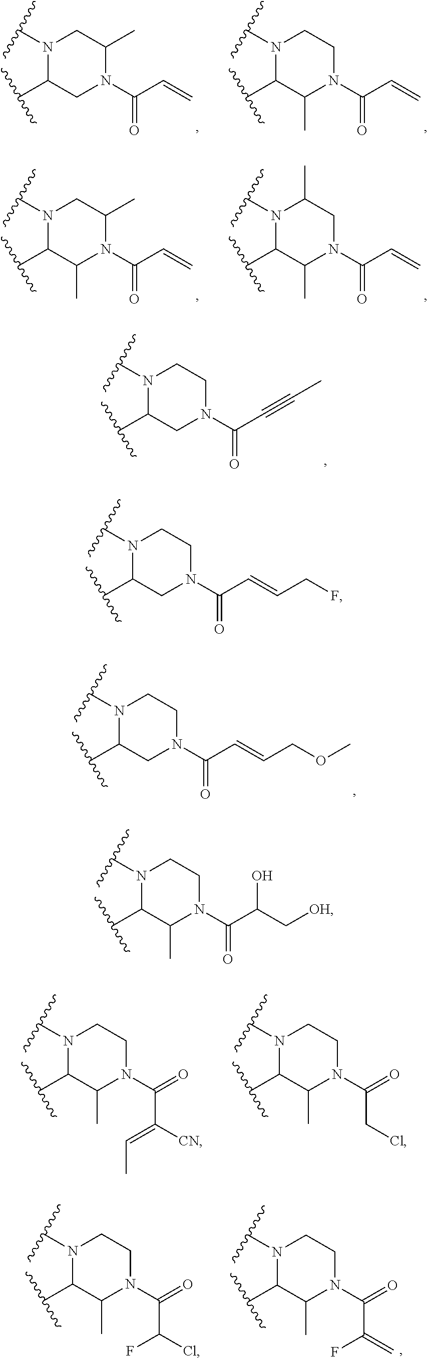

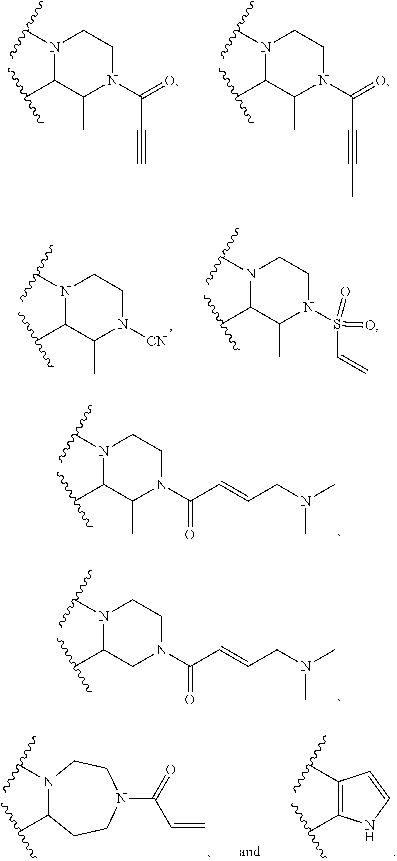

63. The compound of any one of claims 60-62, wherein 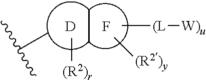
is selected from:

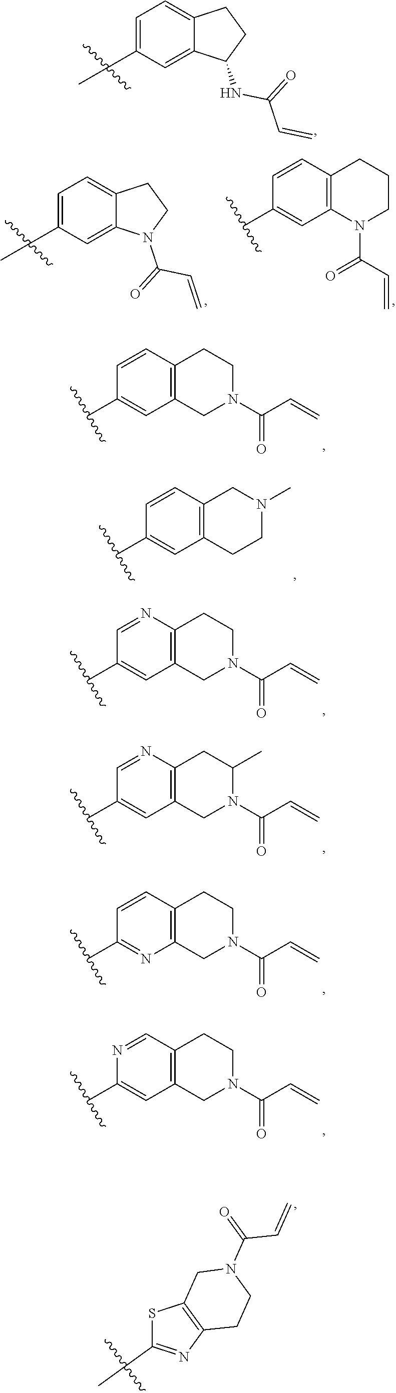

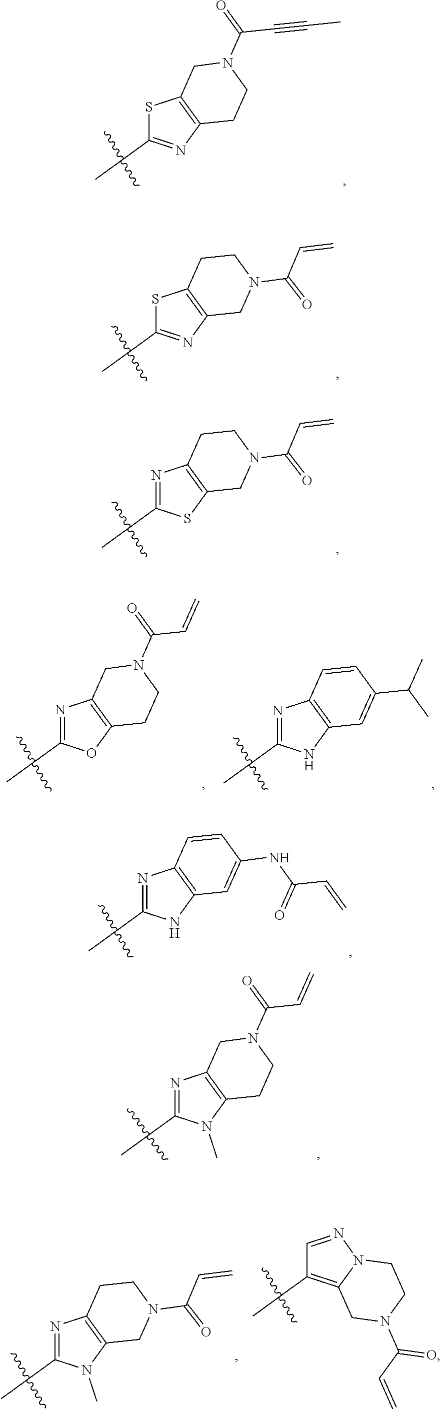

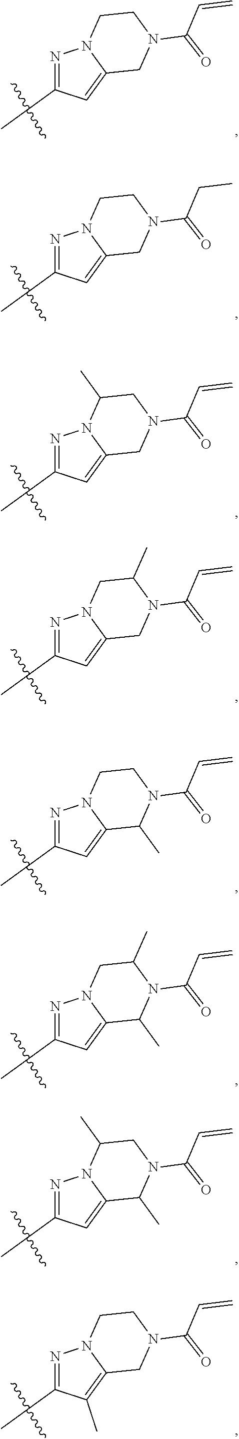

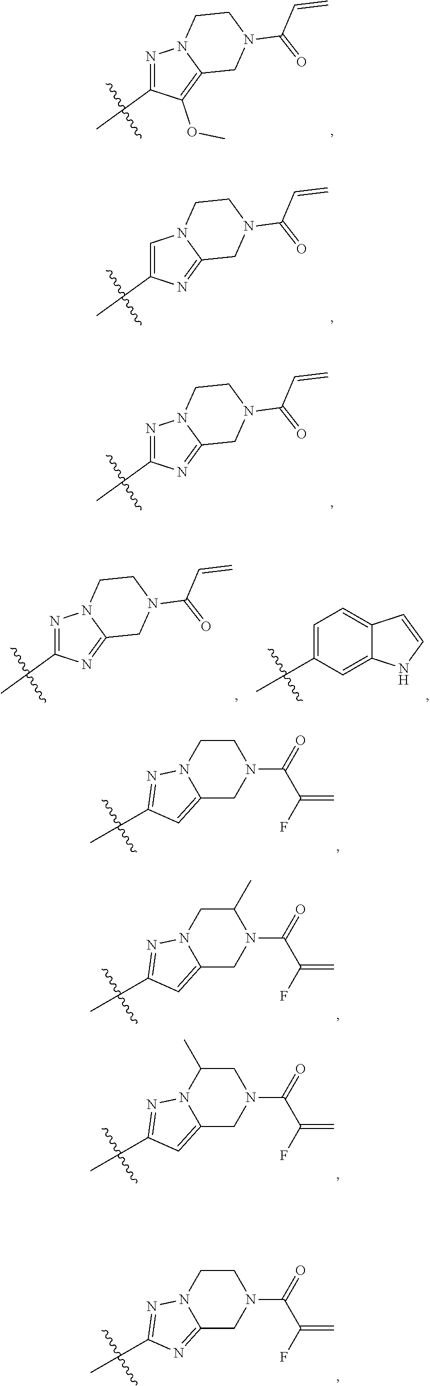

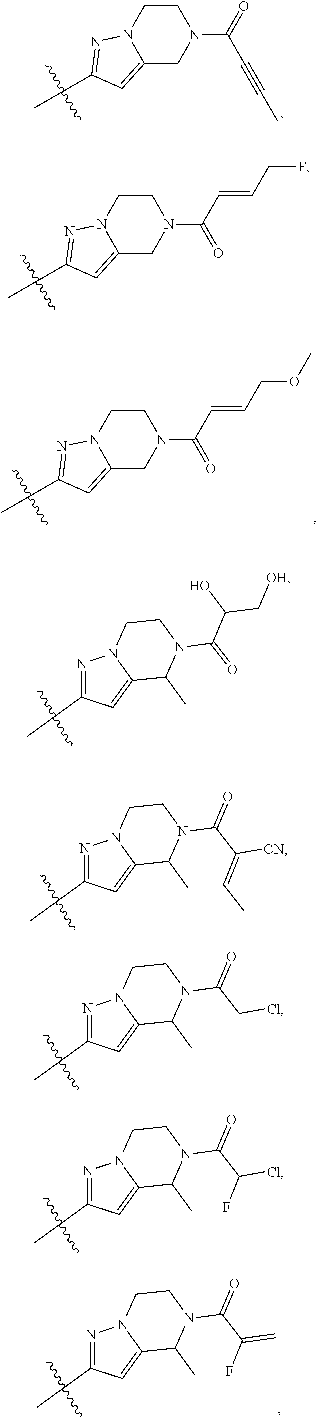

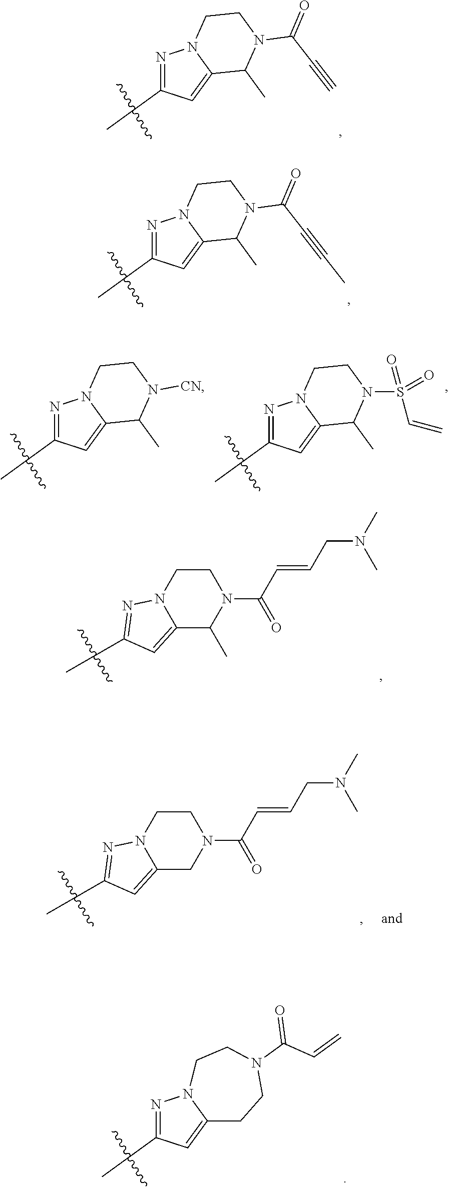

64. The compound of any one of claims 59-63, wherein u is 1.

65. The compound of any one of claims 59-64, wherein each R^{2′} is independently selected from C₁₋₆ alkyl.

66. The compound of any one of claims 59-65, wherein y is 0.

67. The compound of any one of claims 59-65, wherein y is 1 or 2.

68. The compound of any one of claims 1-58, wherein Ring D is not fused to Ring F.

69. The compound of claim 68, wherein Ring D, substituted with r instances of R² and q instances of R¹, is selected from:

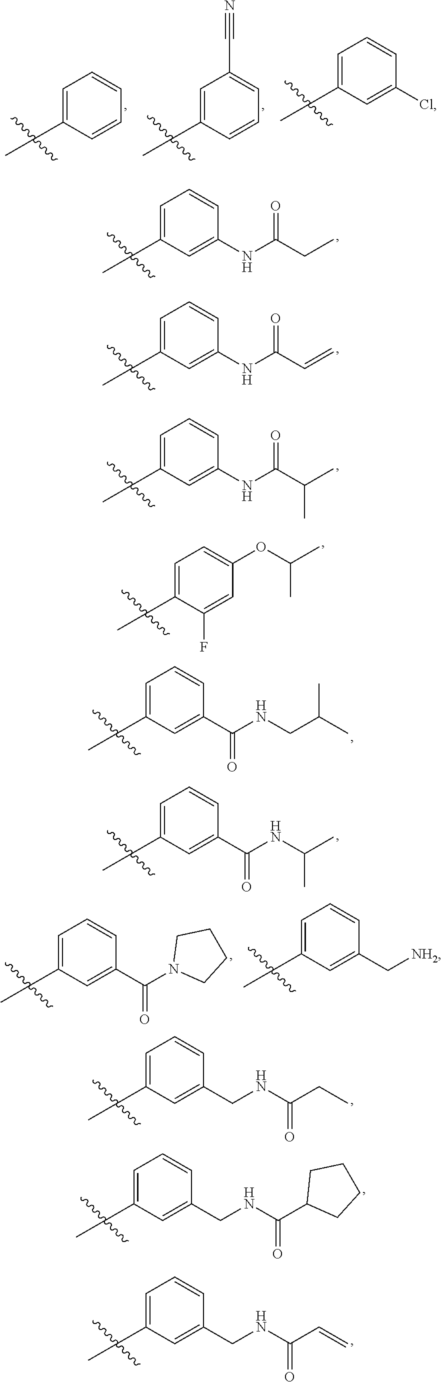

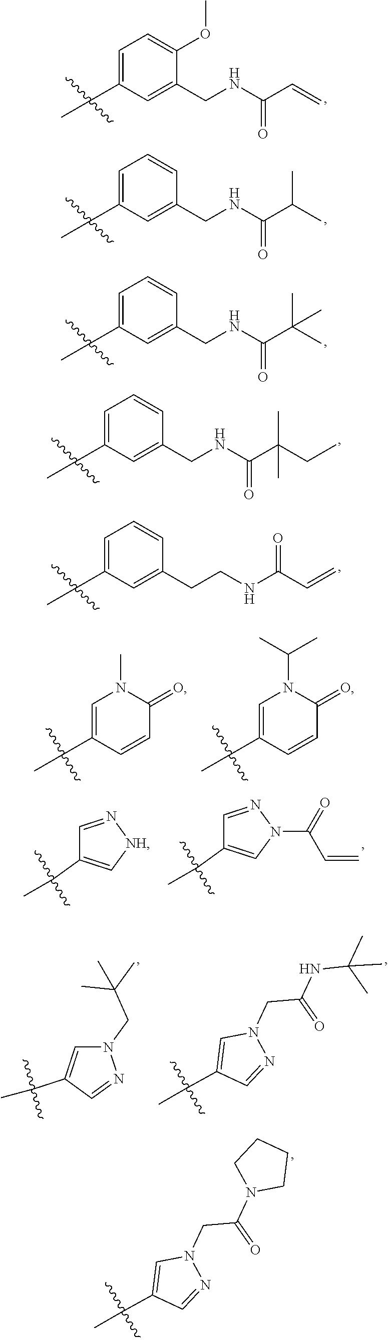

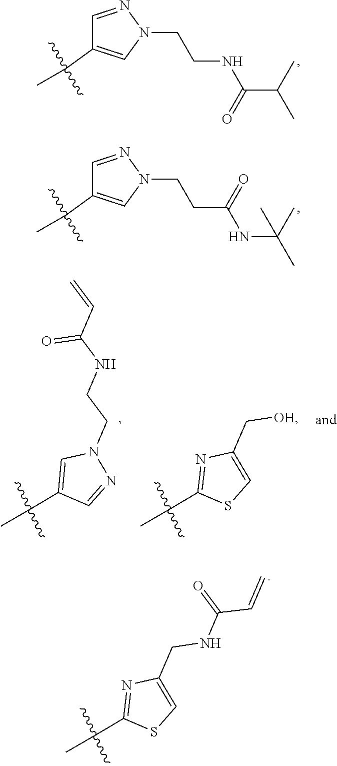 [SMILES: *c1cnn(CCNC(=O)C(C)C)c1 ; *c1cnn(CCC(=O)NC(C)(C)C)c1 ; *c1cnn(CCNC(=O)C=C)c1 ; *c1nc(CO)cs1 ; *c1nc(CNC(=O)C=C)cs1]

70. The compound of claim 68, wherein R¹ is -L-W.

71. The compound of claim 68, wherein R¹ is Ring D′ or a bivalent C₁₋₆ aliphatic chain substituted with Ring D′.

72. The compound of claim 71, wherein Ring D′ is selected from a 4- to 6-membered carbocyclic ring, wherein Ring D′ is substituted with t instances of -L-W.

73. The compound of claim 71, wherein Ring D′ is selected from a 4- to 6-membered heterocyclic ring having 1-2 heteroatoms independently selected from nitrogen, oxygen, and sulfur, wherein Ring D′ is substituted with t instances of -L-W.

74. The compound of any one of claims 71-73, wherein Ring D′, substituted with t instances of -L-W, is selected from:

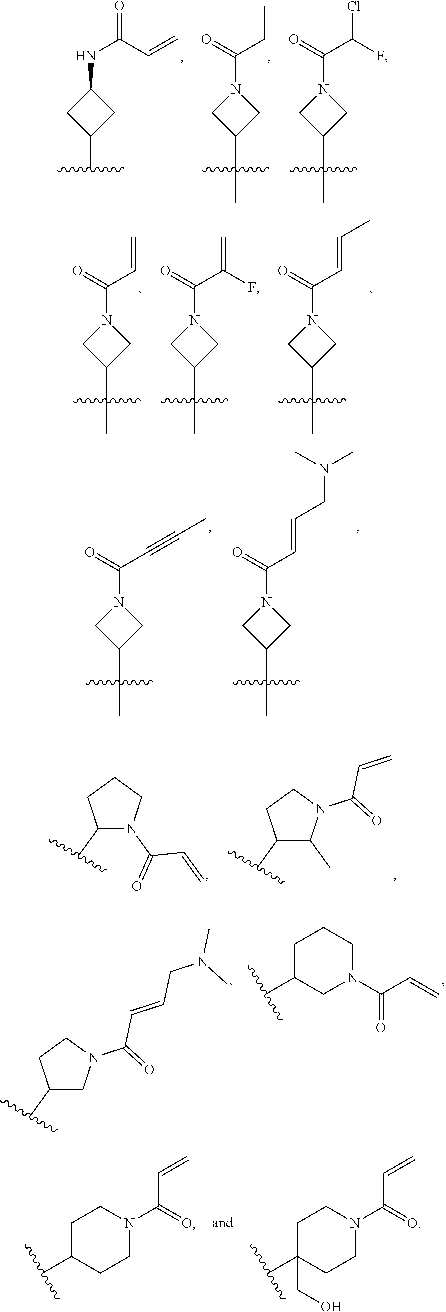

75. The compound of any one of claims 71-74, wherein Ring D, substituted with r instances of R², is selected from:

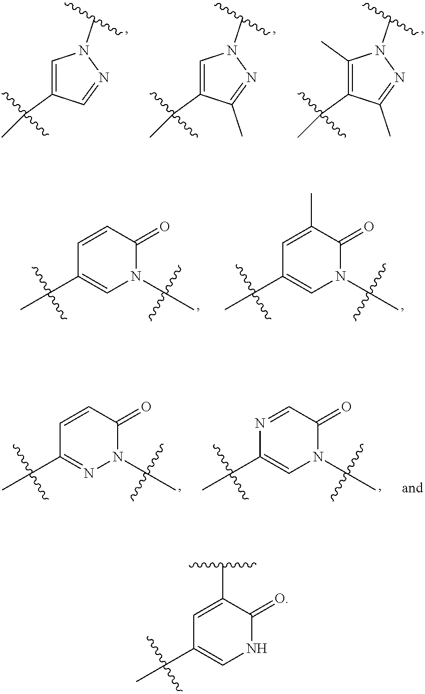

76. The compound of any one of claims 71-73, wherein Ring D is substituted with 1 instance of R¹, R¹ is Ring D′, and 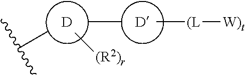
is selected from:

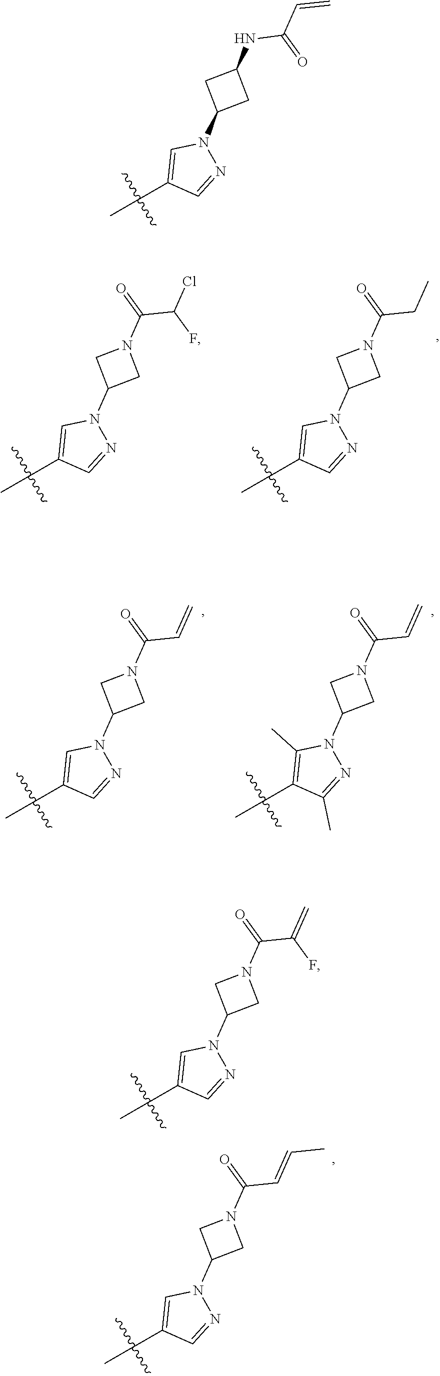

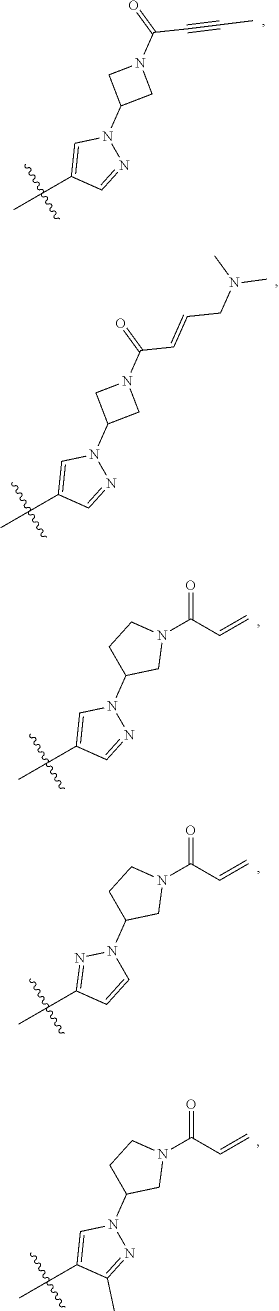 [SMILES: *c1cnn(C2CN(C(=O)C#CC)C2)c1 ; *c1cnn(C2CN(C(=O)/C=C/CN(C)C)C2)c1 ; *c1cnn(C2CCN(C(=O)C=C)C2)c1 ; *c1ccn(C2CCN(C(=O)C=C)C2)n1 ; *c1cn(C2CCN(C(=O)C=C)C2)nc1C]

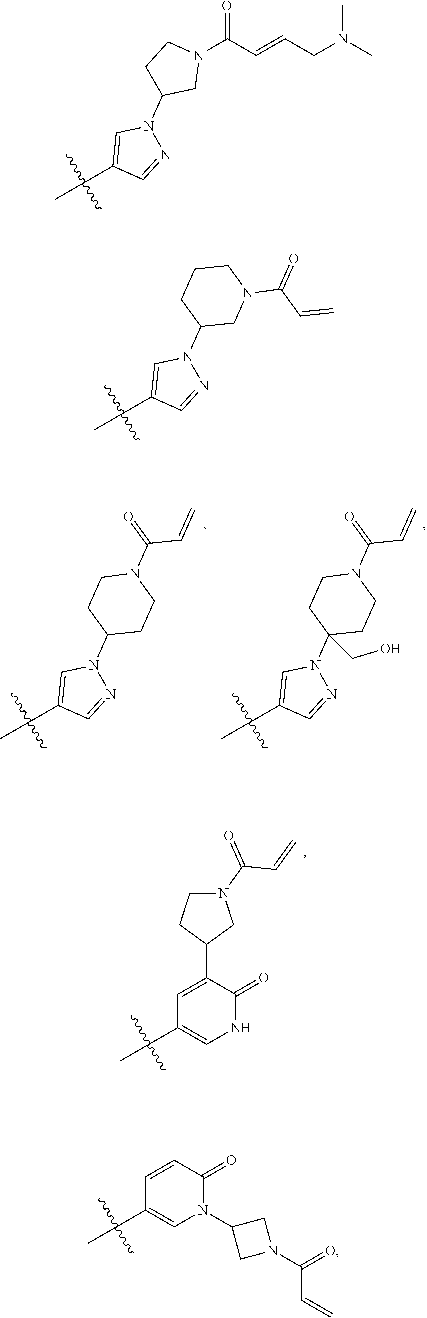 [SMILES: *c1cnn(C2CCN(C(=O)/C=C/CN(C)C)C2)c1 ; *c1cnn(C2CCCN(C(=O)C=C)C2)c1 ; *c1cnn(C2CCN(C(=O)C=C)CC2)c1 ; *c1cnn(C2(CO)CCN(C(=O)C=C)CC2)c1 ; *c1c[nH]c(=O)c(C2CCN(C(=O)C=C)C2)c1 ; *c1ccc(=O)n(C2CN(C(=O)C=C)C2)c1]

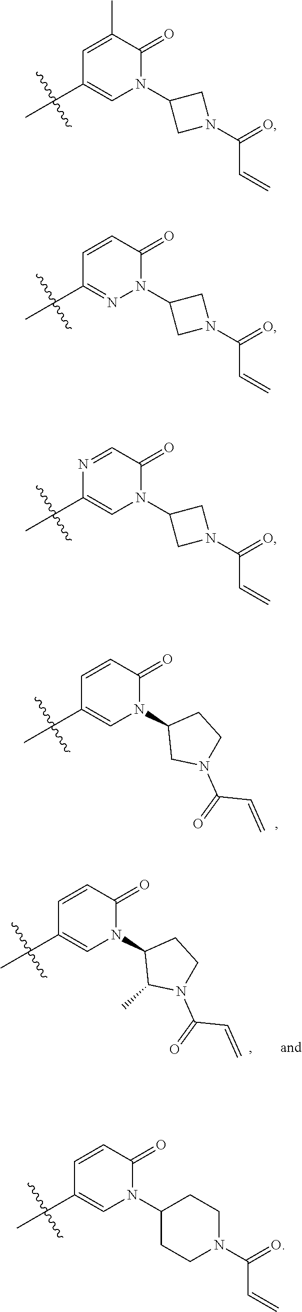 [SMILES: *c1cc(C)c(=O)n(C2CN(C(=O)C=C)C2)c1 ; *c1ccc(=O)n(C2CN(C(=O)C=C)C2)n1 ; *c1cn(C2CN(C(=O)C=C)C2)c(=O)cn1 ; *c1ccc(=O)n([C@H]2CCN(C(=O)C=C)C2)c1 ; *c1ccc(=O)n([C@H]2CCN(C(=O)C=C)[C@@H]2C)c1 ; *c1ccc(=O)n(C2CCN(C(=O)C=C)CC2)c1]

77. The compound of any one of claims 68-76, wherein q is 1.

78. The compound of any one of claims 1-77, wherein r is 0.

79. The compound of any one of claims 1-77, wherein r is 1 or 2.

80. The compound of any one of claims 1-79, wherein t is 0.

81. The compound of any one of claims 1-79, wherein t is 1.

82. The compound of any one of claims 1-81, wherein each L is independently selected from a bivalent straight or branched C₁₋₈ aliphatic chain, wherein one or two methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.

83. The compound of claim 1-82, wherein each L is independently selected from a bivalent straight or branched C₁₋₈ aliphatic chain having one or more units of unsaturation, wherein one or more methylene units of the aliphatic chain are optionally and independently replaced by a group selected from —N(R)—, —O—, —S—, —C(O)—, —SO₂—, —CH(X)—, —C(X)₂—, —C(O)N(R)—, —N(R)C(O)—, —C(O)O—, —OC(O)—, —SO₂N(R)—, and —N(R)SO₂—.

84. The compound of claim 83, wherein each L is independently selected from a bivalent straight or branched C₁₋₈ aliphatic chain having one double bond, wherein one or two methylene units of the aliphatic chain are optionally and independently replaced by a group selected from —N(R)—, —O—, —C(O)—, —SO₂—, —C(O)N(R)—, —N(R)C(O)—, —C(O)O—, —OC(O)—, —SO₂N(R)—, and —N(R)SO₂—.

85. The compound of any one of claims 1-84, wherein each L is independently selected from a bivalent straight or branched C₁₋₈ aliphatic chain, wherein one or two methylene units of the aliphatic chain are optionally and independently replaced by a group selected from —N(R)—, —O—, C(O)—, —C(O)N(R)—, and —N(R)C(O)—.

86. The compound of any one of claims 1-85, wherein each L is independently selected from a bivalent straight or branched C₁₋₄ aliphatic chain.

87. The compound of any one of claims 1-86, wherein each L is independently selected from a bivalent straight or branched C₁₋₄ aliphatic chain having one double bond, wherein one or two methylene units of the aliphatic chain are optionally and independently replaced by a group selected from —N(R)—, —C(O)—, and —N(R)C(O)—.

88. The compound of any one of claims 1-87, wherein each L is independently selected from —C(O)CClF—, —C(O)CH═CH—, —N(R)C(O)CH═CH—, —C(O)C(≡CH₂)—, —C(O)CH═CH—CH₂—, —C(O)CH═CHCH₂OCH₂—, —C(O)CH═CHCH₂N(R)—, —CH₂N(R)C(O)CH═CH—, and —CH₂CH₂N(R)C(O)CH═CH—.

89. The compound of any one of claims 1-88, wherein each W is hydrogen.

90. The compound of any one of claims 1-88, wherein each W is a halogen.

91. The compound of any one of claims 1-90, wherein each -L-W is independently selected from —CH₃, —CH(CH₃)₂, —CH₂C(CH₃)₃, —CH₂OH, —CH₂NH₂, —OCH(CH₃)₂, —CN,

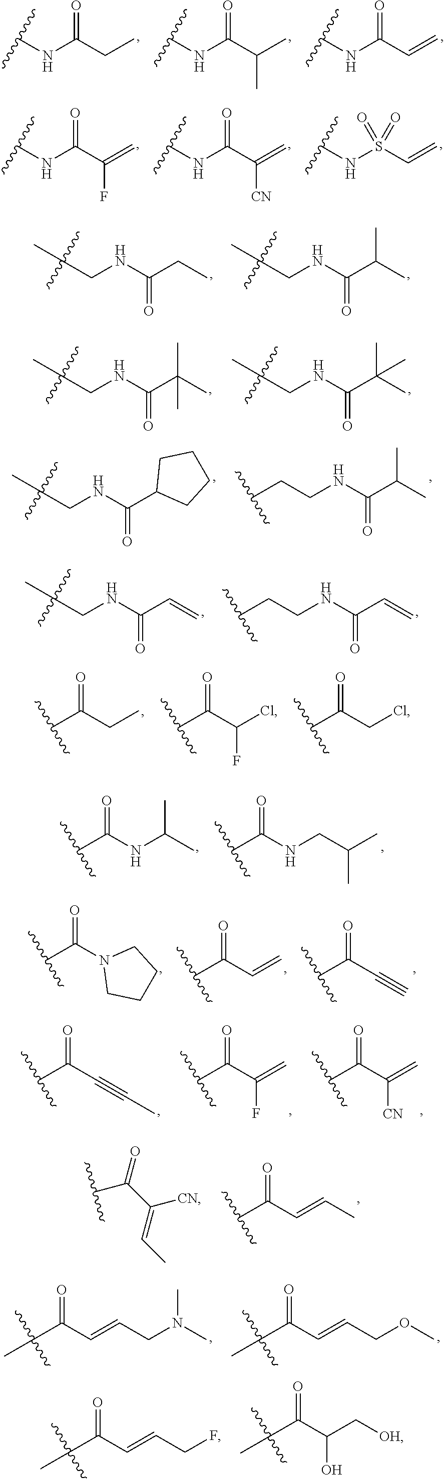

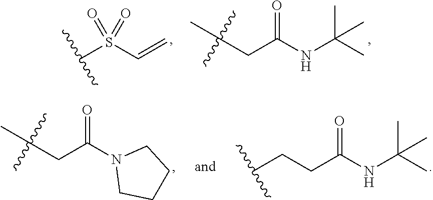

92. The compound of any one of claims 1-91, wherein each -L-W is independently selected from —C(O)CH═CH₂, —C(O)CF=CH₂, —NHC(O)CF=CH₂, and —NHC(O)CH═CH₂.

93. The compound of any one of claims 1-92, wherein R¹ is selected from —CH₃, —CH(CH₃)₂, —CH₂C(CH₃)₃, —CH₂NH₂, —CH₂OH, —OCH(CH₃)₂,

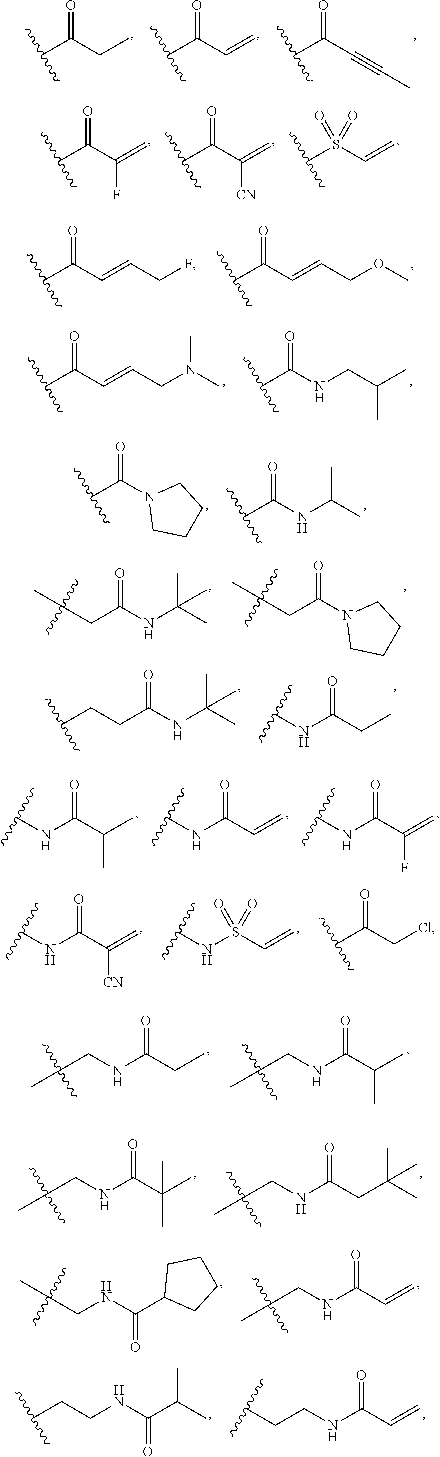

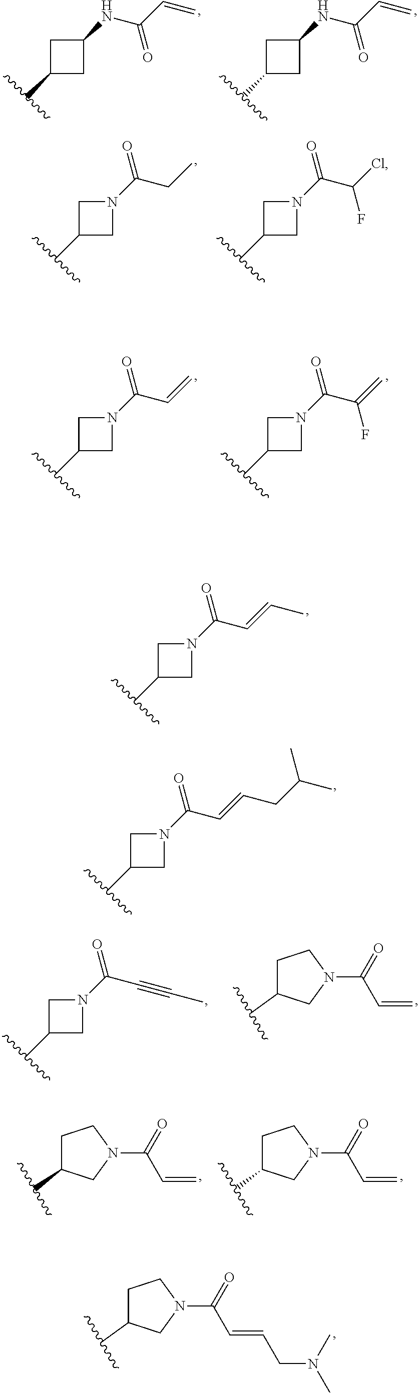

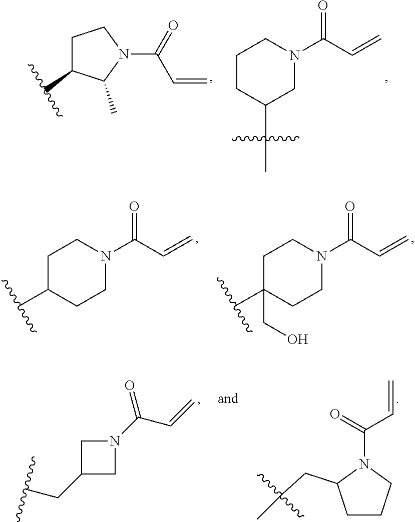

94. The compound according to any one of claims 1-93, wherein each R is independently hydrogen, C₁₋₆ aliphatic, or C₁₋₆ haloaliphatic.

95. A compound selected from Table 1, or a salt (e.g., pharmaceutically acceptable salt) thereof.

96. A compound selected from Table 2, or a salt (e.g., pharmaceutically acceptable salt) thereof.

97. A pharmaceutical composition comprising a compound according to any one of claims 1-96, or a salt (e.g., pharmaceutically acceptable salt) thereof, and a pharmaceutically acceptable carrier or excipient.

98. A method comprising administering a therapeutically effective amount of a compound according to any one of claims 1-96 or a pharmaceutically acceptable salt thereof to a subject in need thereof.

99. The method of claim 98, wherein the subject has a disease, disorder, or condition ameliorated by disruption, inhibition, and/or prevention of an interaction between a small GTPase and a PI3Kα protein.

100. The method of claim 99, wherein the small GTPase is Rac1, CDC42, or a RAS protein.

101. The method of claim 100, wherein the small GTPase is a RAS protein.

102. The method of claim 101, wherein the RAS protein is KRAS, NRAS, HRAS, RRAS, RRAS2, MRAS, or RIT1.

103. The method of any one of claims 98-102, wherein the subject has a cancer.

104. A method of treating a cancer, comprising administering to a subject in need thereof a therapeutically effective amount of a compound according to any one of claims 1-96 or a pharmaceutically acceptable salt thereof.

105. The method of claim 103 or 104, wherein the cancer is associated with and/or characterized by aberrant activation of PI3Kα and/or a mutation in PI3Kα.

106. The method of claim 105, wherein the PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation.

107. The method of claim 106, wherein the PI3Kα protein comprises a E542K, E545K, H1047R, and/or H1047L mutation.

108. The method of any one of claims 103-107, wherein the cancer is characterized by a mutation in a RAS protein.

109. The method of claim 108, wherein the RAS protein comprises a mutation in codon 12, 13, or 61.

110. The method of claim 108 or 109, wherein the RAS protein is KRAS.

111. The method of claim 110, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

112. The method of claim 111, wherein the KRAS protein comprises a G12C or G12D mutation.

113. The method according to claim 108 or 109, wherein the RAS protein is HRAS.

114. The method according to claim 113, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.

115. The method according to claim 108 or 109, wherein the RAS protein is NRAS.

116. The method according to claim 115, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

117. The method according to any one of claims 103-116, wherein the cancer is selected from pancreatic cancer; colon cancer; rectal cancer; colorectal cancer; bowel cancer; breast cancer; ovarian cancer; endometrial cancer; lung cancer; prostate cancer; cancers of the oral cavity and pharynx (lip, tongue, mouth, larynx, pharynx), esophagus, stomach, small intestine, large intestine, liver and biliary passages, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease; non-Hodgkin's lymphomas; multiple myeloma; and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML), and lymphomas including lymphocytic, granulocytic and monocytic lymphomas.

118. The method according to claim 117, wherein the cancer is selected from breast cancer, lung cancer (e.g., non-small cell lung cancer), endometrial cancer, esophageal cancer, ovarian cancer, colorectal cancer, gastric cancer, squamous cell carcinoma, prostate cancer, and pancreatic cancer.

119. The method according to any one of claims 103-118, wherein the cancer is characterized by mutated, overexpressed, and/or amplified receptor tyrosine kinases (e.g., HER family, Met, FGFR, Alk, PDGF, EGFR, or ROS kinases).

120. The method according to any one of claims 103-119, wherein the cancer is characterized by a mutation in or a deletion of a PTEN protein.

121. The method according to any one of claims 98-120, wherein the subject has previously undergone a treatment regimen for cancer.

122. The method according to any one of claims 98-121, wherein the subject has previously entered remission from cancer.

123. A method of treating a metabolic disorder, comprising administering to a subject in need thereof a therapeutically effective amount of a compound according to any one of claims 1-96 or a pharmaceutically acceptable salt thereof.

124. The method according to claim 123, wherein the metabolic disorder is selected from hyperinsulinemia and type 2 diabetes.

125. A method of treating a RASopathy, comprising administering to a subject in need thereof a therapeutically effective amount of a compound according to any one of claims 1-96 or a pharmaceutically acceptable salt thereof.

126. The method according to claim 125, wherein the RASopathy is selected from neurofibromatosis type 1 (NF1), capillary malformation-arteriovenous malformation syndrome, and Legius syndrome.

127. A method of treating a vascular disorder or condition, comprising administering to a subject in need thereof a therapeutically effective amount of a compound according to any one of claims 1-96 or a pharmaceutically acceptable salt thereof.

128. The method according to claim 127, wherein the vascular disorder or condition is selected from PIK3CA-related overgrowth syndrome (PROS) and vascular malformations (e.g., venous malformations; lymphatic malformations; congenital lipomatous overgrowth with vascular, epidermal, and skeletal anomalies syndrome (CLOVES); Klippel-Trenaunay Syndrome; PTEN hamartoma tumor syndrome (PHTS); and fibro-adipose vascular anomaly (FAVA)).

129. A method of disrupting, inhibiting, and/or preventing an interaction between a small GTPase and a PI3Kα protein in a subject, comprising administering to the subject a compound according to any one of claims 1-96 or a pharmaceutically acceptable salt thereof.

130. A method of disrupting, inhibiting, and/or preventing an interaction between a small GTPase and a PI3Kα protein, comprising contacting a cell containing the small GTPase and the PI3Kα protein with a compound according to any one of claims 1-96 or a pharmaceutically acceptable salt thereof.

131. A method comprising contacting a cell containing a small GTPase and a PI3Kα protein with a compound according to any one of claims 1-96 or a pharmaceutically acceptable salt thereof.

132. The method of claim 130 or 131, wherein the cell is included in a subject.

133. The method of any one of claims 129-132, wherein the small GTPase is selected from Rac1, CDC42, and a RAS protein.

134. The method of claim 133, wherein the small GTPase is a RAS protein.

135. The method of claim 134, wherein the RAS protein is selected from HRAS, NRAS, KRAS, RRAS, RRAS2, MRAS, and RIT1.

136. The method of claim 135, wherein the RAS protein is KRAS.

137. The method of claim 136, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

138. The method of claim 137, wherein the KRAS protein comprises a G12C or G12D mutation.

139. The method according to claim 135, wherein the RAS protein is HRAS.

140. The method according to claim 139, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.

141. The method according to claim 135, wherein the RAS protein is NRAS.

142. The method according to claim 141, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

143. A compound according to any one of claims 1-96 or a pharmaceutically acceptable salt thereof for use as a medicament.

144. Use of a compound according to any one of claims 1-96 or a pharmaceutically acceptable salt thereof in the manufacture of a medicament.

145. The compound or use according to claim 143 or 144, wherein the medicament is for treating a cancer.

146. The compound or use according to claim 145, wherein the cancer is associated with and/or characterized by aberrant activation of PI3Kα and/or a mutation in PI3Kα.

147. The compound or use according to claim 146, wherein the PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation.

148. The compound or use according to claim 147, wherein the PI3Kα protein comprises a E542K, E545K, H1047R, and/or H1047L mutation.

149. The compound or use according to any one of claims 145-148, wherein the cancer is characterized by a mutation in a RAS protein.

150. The compound or use according to claim 149, wherein the RAS protein comprises a mutation in codon 12, 13, or 61.

151. The compound or use according to claim 149 or 150, wherein the RAS protein is KRAS.

152. The compound or use according to claim 151, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

153. The compound or use according to claim 152, wherein the KRAS protein comprises a G12C or G12D mutation.

154. The compound or use according to claim 149 or 150, wherein the RAS protein is HRAS.

155. The compound according to claim 154, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.

156. The compound or use according to claim 149 or 150, wherein the RAS protein is NRAS.

157. The compound or use according to claim 156, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

158. The compound or use according to any one of claims 145-157, wherein the cancer is selected from pancreatic cancer; colon cancer; rectal cancer; colorectal cancer; bowel cancer; breast cancer; ovarian cancer; endometrial cancer; lung cancer; prostate cancer; cancers of the oral cavity and pharynx (lip, tongue, mouth, larynx, pharynx), esophagus, stomach, small intestine, large intestine, liver and biliary passages, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease; non-Hodgkin's lymphomas; multiple myeloma; and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML), and lymphomas including lymphocytic, granulocytic and monocytic lymphomas.

159. The compound or use according to claim 158, wherein the cancer is selected from breast cancer, lung cancer (e.g., non-small cell lung cancer), endometrial cancer, esophageal cancer, ovarian cancer, colorectal cancer, gastric cancer, squamous cell carcinoma, prostate cancer, and pancreatic cancer.

160. The compound or use according to any one of claims 145-159, wherein the cancer is characterized by mutated, overexpressed, and/or amplified receptor tyrosine kinases (e.g., HER family, Met, FGFR, Alk, PDGF, EGFR, or ROS kinases).

161. The compound or use according to any one of claims 145-159, wherein the cancer is characterized by a mutation in or a deletion of a PTEN protein.

162. The compound or use according to claim 143 or 144, wherein the medicament is for treating a metabolic disorder, a RASopathy, or a vascular disorder.

163. The compound or use according to claim 162, wherein: (i) the metabolic disorder is selected from hyperinsulinemia and type 2 diabetes; (ii) the RASopathy is selected from neurofibromatosis type 1 (NF1), capillary malformation-arteriovenous malformation syndrome, and Legius syndrome; and/or (iii) the vascular disorder or condition is selected from PIK3CA-related overgrowth syndrome (PROS) and vascular malformations (e.g., venous malformations; lymphatic malformations; congenital lipomatous overgrowth with vascular, epidermal, and skeletal anomalies syndrome (CLOVES); Klippel-Trenaunay Syndrome; PTEN hamartoma tumor syndrome (PHTS); or fibro-adipose vascular anomaly (FAVA)).

164. A compound according to any one of claim 1-96 or a pharmaceutically acceptable salt thereof for use in treating a disease, disorder, or condition.

165. The compound for use according to claim 164 for use in treating a cancer.

166. The compound for use according to claim 165, wherein the cancer is associated with and/or characterized by aberrant activation of PI3Kα and/or a mutation in PI3Kα.

167. The compound for use according to claim 166, wherein the PI3Kα protein comprises a N345K, E726K, C420R, Q546R, G118D, E453K, Q546K, G1049R, M10431, K111E, K111N, E81K, E545A, E545G, N1044K, E110del, Q546P, E542K, E545K, H1047R, and/or H1047L mutation.

168. The compound for use according to claim 167, wherein the PI3Kα protein comprises a E542K, E545K, H1047R, and/or H1047L mutation.

169. The compound for use according to any one of claims 165-168, wherein the cancer is characterized by a mutation in a RAS protein.

170. The compound for use according to claim 169, wherein the RAS protein comprises a mutation in codon 12, 13, or 61.

171. The compound for use according to claim 169 or 170, wherein the RAS protein is KRAS.

172. The compound for use according to claim 171, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

173. The compound for use according to claim 172, wherein the KRAS protein comprises a G12C or G12D mutation.

174. The compound for use according to claim 169 or 170, wherein the RAS protein is HRAS.

175. The compound for use according to claim 174, wherein the HRAS protein comprises a G12C, G12D, G12S, G12V, G12R, G12A, G13D, G13C, G13R, G13S, G13V, Q61K, Q61L, Q61P, Q61R, and/or Q611H mutation.

176. The compound for use according to claim 169 or 170, wherein the RAS protein is NRAS.

177. The compound for use according to claim 176, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

178. The compound for use according to any one of claims 165-177, wherein the cancer is selected from pancreatic cancer; colon cancer; rectal cancer; colorectal cancer; bowel cancer; breast cancer; ovarian cancer; endometrial cancer; lung cancer; prostate cancer; cancers of the oral cavity and pharynx (lip, tongue, mouth, larynx, pharynx), esophagus, stomach, small intestine, large intestine, liver and biliary passages, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; cancers of the thyroid and other endocrine glands; Hodgkin's disease; non-Hodgkin's lymphomas; multiple myeloma; and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML), and lymphomas including lymphocytic, granulocytic and monocytic lymphomas.

179. The compound for use according to claim 178, wherein the cancer is selected from breast cancer, lung cancer (e.g., non-small cell lung cancer), endometrial cancer, esophageal cancer, ovarian cancer, colorectal cancer, gastric cancer, squamous cell carcinoma, prostate cancer, and pancreatic cancer.

180. The compound for use according to any one of claims 165-179, wherein the cancer is characterized by mutated, overexpressed, and/or amplified receptor tyrosine kinases (e.g., HER family, Met, FGFR, Alk, PDGF, EGFR, or ROS kinases).

181. The compound for use according to any one of claims 165-180, wherein the cancer is characterized by a mutation in or a deletion of a PTEN protein.

182. The compound for use according to claim 164 for use in treating a metabolic disorder, a RASopathy, or a vascular disorder.

183. The compound for use according to claim 182, wherein: (i) the metabolic disorder is selected from hyperinsulinemia and type 2 diabetes; (ii) the RASopathy is selected from neurofibromatosis type 1 (NF1), capillary malformation-arteriovenous malformation syndrome, and Legius syndrome; and/or (iii) the vascular disorder or condition is selected from PIK3CA-related overgrowth syndrome (PROS) and vascular malformations (e.g., venous malformations; lymphatic malformations; congenital lipomatous overgrowth with vascular, epidermal, and skeletal anomalies syndrome (CLOVES); Klippel-Trenaunay Syndrome; PTEN hamartoma tumor syndrome (PHTS); and fibro-adipose vascular anomaly (FAVA)).

184. A compound capable of disrupting, inhibiting, and/or preventing an interaction between a small GTPase and a PI3Kα protein.

185. The compound of claim 184, wherein the compound is capable of binding to PI3Kα, such that (i) the interaction between the small GTPase and PI3Kα is at least partially disrupted, prevented, or inhibited; and/or (ii) the kinase activity of PI3Kα is not significantly inhibited.

186. The compound of claim 184 or 185, wherein the compound has an activity of:
(i) <5 μM or ≥5 μM and ≤25 μM in the assay of Biological Example 1 (e.g., a surface plasmon resonance (SPR) binding assay assessing inhibition of the KRAS-PI3Kα interaction);
(ii) ≥75%, <75% and ≥50%, or <50% and ≥25% in the assay of Biological Example 2 (e.g., a Matrix-Assisted Laser Desorption Ionization-Time of Flight Mass Spectrometry (MALDI-TOF MS) analysis of covalent modification of Cys242 in human PIK3CA (157-299);
(iii) <1 μM or ≥1 μM and ≤10 μM in the assay of Biological Example 3 (e.g., a matrix-assisted cell-based pAKT homogenous time-resolved fluorescence (HTRF) assay in Tet-on KRAS G12D HeLa cells); and/or
(iv) <0.1 μM, ≥0.1 μM and <1 μM, or ≥1 μM and ≤3 μM in the assay of Biological Example 4 (e.g., a matrix-assisted cell-based pAKT HTRF assay in BT474 cells).

187. The compound of any one of claims 184-186, wherein the compound comprises an electrophilic moiety.

188. The compound of any one of claims 184-187, wherein the compound is capable of interacting with a Cys242 residue in the catalytic subunit of PI3Kα.

189. The compound of any one of claims 184-188, wherein the compound is capable of irreversibly binding the PI3Kα protein.

190. The compound of any one of claims 184-189, wherein the compound is capable of reversibly binding the PI3Kα protein.

191. The compound of any one of claims 184-190, wherein the small GTPase is Rac1, CDC42, or a RAS protein.

192. The compound of claim 191, wherein the RAS protein is KRAS, NRAS, HRAS, RRAS, RRAS2, MRAS, or RIT1.

193. The compound of claim 191 or 192, wherein the RAS protein comprises a mutation in codon 12, 13, or 61.

194. The compound of claim 192 or 193, wherein the RAS protein is KRAS.

195. The compound of claim 194, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

196. The compound of claim 195, wherein the KRAS protein comprises a G12C or G12D mutation.

197. The compound of claim 192 or 193, wherein the RAS protein is HRAS.

198. The compound of claim 197, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.

199. The compound of claim 192 or 193, wherein the RAS protein is NRAS.

200. The compound of claim 199, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

201. The compound of any one of claims 184-200, wherein the compound is a compound according to any one of claims 1-96.

202. A method of disrupting, inhibiting, and/or preventing an interaction between a small GTPase and a PI3Kα protein, comprising contacting a cell containing the small GTPase and the PI3Kα protein with a compound according to any one of claims 184-201 or a pharmaceutically acceptable salt thereof.

203. The method of claim 202, wherein the cell is within a subject.

204. The method of claim 202 or 203, wherein the small GTPase is Rac1, CDC42, or a RAS protein.

205. The method of claim 204, wherein the RAS protein is KRAS, NRAS, HRAS, RRAS, RRAS2, MRAS, or RIT1.

206. The method of claim 205, wherein the RAS protein comprises a mutation in codon 12, 13, or 61.

207. The method of claim 205 or 206, wherein the RAS protein is KRAS.

208. The method of claim 207, wherein the KRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.

209. The method of claim 208, wherein the KRAS protein comprises a G12C or G12D mutation.

210. The method of claim 205 or 206, wherein the RAS protein is HRAS.

211. The method of claim 210, wherein the HRAS protein comprises a G12C, G12D, G12S, G12V, G12R, G12A, G13D, G13C, G13R, G13S, G13V, Q61K, Q61L, Q61P, Q61R, and/or Q61H mutation.

212. The method of claim 205 or 206, wherein the RAS protein is NRAS.

213. The method of claim 212, wherein the NRAS protein comprises a G12C, G12D, G12S, G12V, G12R, G12A, G13D, G13A, G13C, G13R, G13S, G13V, Q61E, Q61K, Q61L, Q61P, Q61R, and/or Q61H mutation.
